(19) United States
(12) Patent Application Publication
(10) Pub. No.: US 2011/0097330 A1
(43) Pub. Date: Apr. 28, 2011

(54) Novel Gene Disruptions, Compostitions and Methods Relating Thereto

(75) Inventors: Allison Anne Byers Horner, Missouri City, TX (US); Catherine Batac Clarke, Conroe, TX (US); Katherin E. Combs, San Antonio, TX (US); Frederic J. de Sauvage, Foster City, CA (US); Joel Edwards, Rancho Santa Margarita, CA (US); Paul Godowski, Hillsborough, CA (US); Deanna Grant Wilson, San Mateo, CA (US); Wenhu Huang, San Diego, CA (US); Lorelei Diane Ketcherside, The Woodlands, TX (US); Erin Marie Massey, Spring, TX (US); Charles Montgomery, Jay, OK (US); Bobby Joe Payne, The Woodlands, TX (US); Andrew Peterson, San Francisco, CA (US); Ni Nancy Qian, San Diego, CA (US); Jeffrey J. Schrick, Conroe, TX (US); Zheng-Zheng Shi, Spring, TX (US); Mary Jean Sparks, Corpus Christi, TX (US); Joy Anne Stala, Santa Monica, CA (US); Colleen M. Viator, Katy, TX (US); Peter Vogel, Spring, TX (US); Weilan Ye, Foster City, CA (US); Jung-Hua Yeh, Belmont, CA (US); Zhiyong Ding, Valhalla, NY (US)

(73) Assignees: Genentech, Inc., South San Francisco, CA (US); Lexicon Pharmaceuticals, Inc., The Woodlands, TX (US)

(21) Appl. No.: 11/722,967

(22) PCT Filed: Feb. 27, 2006
(86) PCT No.: PCT/US2006/007353

§ 371 Date: Aug. 9, 2010

Related U.S. Application Data
(60) Provisional application No. 60/661,173, filed on Mar. 11, 2005.
(60) Provisional application No. 60/740,522, filed on Nov. 29, 2005.

(51) Int. Cl. A61K 49/00; C07K 16/18; A61K 39/00; A61P 25/00; A61P 9/00; A61P 37/00; A61P 27/02; A61P 25/24; A61P 25/22; A61P 25/18; A61P 27/06; A61P 27/12; A61P 9/10; A61P 19/02; A61P 19/10; C12Q 1/68; C12N 5/071; C12N 5/0735; C12Q 1/02

(52) U.S. Cl. 424/139.1; 530/387.9; 435/6; 800/3; 435/325; 435/354; 435/29

(57) ABSTRACT

The present invention relates to transgenic animals, as well as compositions and methods relating to the characterization of gene function. Specifically, the present invention provides transgenic mice comprising disruptions in PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 genes. Such in vivo studies and characterizations may provide valuable identification and discovery of therapeutics and/or treatments useful in the prevention, amelioration or correction of diseases or dysfunctions associated with gene disruptions such as neurological disorders; cardiovascular, endothelial or angiogenic disorders; eye abnormalities; immunological disorders; oncological disorders; bone metabolic abnormalities or disorders; lipid metabolic disorders; or developmental abnormalities.

### FIELD OF THE INVENTION

The present invention relates to compositions, including transgenic and knockout animals and methods of using such compositions for the diagnosis and treatment of diseases or disorders.

### BACKGROUND OF THE INVENTION

Extracellular proteins play important roles in, among other things, the formation, differentiation and maintenance of multicellular organisms. The fate of many individual cells, e.g., proliferation, migration, differentiation, or interaction with other cells, is typically governed by information received from other cells and/or the immediate environment. This information is often transmitted by secreted polypeptides (for instance, mitogenic factors, survival factors, cytotoxic factors, differentiation factors, neuropeptides, and hormones) which are, in turn, received and interpreted by diverse cell receptors or membrane-bound proteins. These secreted polypeptides or signaling molecules normally pass through the cellular secretory pathway to reach their site of action in the extracellular environment.

Secreted proteins have various industrial applications, including as pharmaceuticals, diagnostics, biosensors and bioreactors. Most protein drugs available at present, such as thrombolytic agents, interferons, interleukins, erythropoietins, colony stimulating factors, and various other cytokines, are secretory proteins. Their receptors, which are membrane proteins, also have potential as therapeutic or diagnostic agents. Efforts are being undertaken by both industry and academia to identify new, native secreted proteins. Many efforts are focused on the screening of mammalian recombinant DNA libraries to identify the coding sequences for novel secreted proteins. Examples of screening methods and techniques are described in the literature [see, for example, Klein et al., Proc. Natl. Acad. Sci. 93:7108-7113 (1996); U.S. Pat. No. 5,536,637)].

Membrane-bound proteins and receptors can play important roles in, among other things, the formation, differentiation and maintenance of multicellular organisms. The fate of many individual cells, e.g., proliferation, migration, differentiation, or interaction with other cells, is typically governed by information received from other cells and/or the immediate environment. This information is often transmitted by secreted polypeptides (for instance, mitogenic factors, survival factors, cytotoxic factors, differentiation factors, neuropeptides, and hormones) which are, in turn, received and interpreted by diverse cell receptors or membrane-bound proteins. Such membrane-bound proteins and cell receptors include, but are not limited to, cytokine receptors, receptor kinases, receptor phosphatases, receptors involved in cell-cell interactions, and cellular adhesion molecules like selectins and integrins. For instance, transduction of signals that regulate cell growth and differentiation is regulated in part by phosphorylation of various cellular proteins. Protein tyrosine kinases, enzymes that catalyze that process, can also act as growth factor receptors. Examples include fibroblast growth factor receptor and nerve growth factor receptor.

Membrane-bound proteins and receptor molecules have various industrial applications, including as pharmaceutical and diagnostic agents. Receptor immuno-adhesions, for instance, can be employed as therapeutic agents to block receptor-ligand interactions. The membrane-bound proteins can also be employed for screening of potential peptide or small molecule inhibitors of the relevant receptor/ligand interaction.

Efforts are being undertaken by both industry and academia to identify new, native receptor or membrane-bound proteins. Many efforts are focused on the screening of mammalian recombinant DNA libraries to identify the coding sequences for novel receptor or membrane-bound proteins.

Given the importance of secreted and membrane-bound proteins in biological and disease processes, in vivo studies and characterizations may provide valuable identification and discovery of therapeutics and/or treatments useful in the prevention, amelioration or correction of diseases or dysfunctions. In this regard, genetically engineered mice have proven to be invaluable tools for the functional dissection of biological processes relevant to human disease, including immunology, cancer, neuro-biology, cardiovascular biology, obesity and many others. Gene knockouts can be viewed as modeling the biological mechanism of drug action by presaging the activity of highly specific antagonists in vivo. Knockout mice have been shown to model drug activity; phenotypes of mice deficient for specific pharmaceutical target proteins can resemble the human clinical phenotype caused by the corresponding antagonist drug. Gene knockouts enable the discovery of the mechanism of action of the target, the predominant physiological role of the target, and mechanism-based side-effects that might result from inhibition of the target in mammals. Examples of this type include mice deficient in the angiotensin converting enzyme (ACE) [Esther, C. R. et al., Lab. Invest., 74:953-965 (1996)] and cyclooxygenase-1 (COX1) genes [Langenbach, R. et al., Cell, 83:483-492 (1995)]. Conversely, knocking the gene out in the mouse can have an opposite phenotypic effect to that observed in humans after administration of an agonist drug to the corresponding target. Examples include the erythropoietin knockout [Wu, C. S. et al., Cell, 83:59-67 (1996)], in which a consequence of the mutation is deficient red blood cell production, and the GABA(A)-R-133 knockout [DeLorey, T. M., J. Neurosci., 18:8505-8514 (1998)], in which the mutant mice show hyperactivity and hyper-responsiveness. Both these phenotypes are opposite to the effects of erythropoietin and benzodiazepine administration in humans. A striking example of a target validated using mouse genetics is the ACC2 gene. Although the human ACC2 gene had been identified several years ago, interest in ACC2 as a target for drug development was stimulated only recently after analysis of ACC2 function using a knockout mouse. ACC2 mutant mice eat more than their wild-type littermates, yet burn more fat and store less fat in their adipocytes, making this enzyme a probable target for chemical antagonism in the treatment of obesity [Abu-Elheiga, L. et al., Science, 291:2613-2616 (2001)].

In the instant application, mutated gene disruptions have resulted in phenotypic observations related to various disease conditions or dysfunctions including: CNS/neurological disturbances or disorders such as anxiety; eye abnormalities and associated diseases; cardiovascular, endothelial or angiogenic disorders including atherosclerosis; abnormal metabolic disorders including diabetes and dyslipidemias associated with elevated serum triglycerides and cholesterol levels; immunological and inflammatory disorders; oncological disorders; bone metabolic abnormalities or disorders such as arthritis, osteoporosis and osteopetrosis; or a developmental disease such as embryonic lethality.

### SUMMARY OF THE INVENTION

### A. Embodiments

The invention provides an isolated nucleic acid molecule comprising a nucleotide sequence that encodes a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide.

In one aspect, the isolated nucleic acid molecule comprises a nucleotide sequence having at least about 80% nucleic acid sequence identity, alternatively at least about 81% nucleic acid sequence identity, alternatively at least about 82% nucleic acid sequence identity, alternatively at least about 83% nucleic acid sequence identity, alternatively at least about 84% nucleic acid sequence identity, alternatively at least about 85% nucleic acid sequence identity, alternatively at least about 86% nucleic acid sequence identity, alternatively at least about 87% nucleic acid sequence identity, alternatively at least about 88% nucleic acid sequence identity, alternatively at least about 89% nucleic acid sequence identity, alternatively at least about 90% nucleic acid sequence identity, alternatively at least about 91% nucleic acid sequence identity, alternatively at least about 92% nucleic acid sequence identity, alternatively at least about 93% nucleic acid sequence identity, alternatively at least about 94% nucleic acid sequence identity, alternatively at least about 95% nucleic acid sequence identity, alternatively at least about 96% nucleic acid sequence identity, alternatively at least about 97% nucleic acid sequence identity, alternatively at least about 98% nucleic acid sequence identity and alternatively at least about 99% nucleic acid sequence identity to (a) a DNA molecule encoding a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide having a full-length amino acid sequence as disclosed herein, an amino acid sequence lacking the signal peptide as disclosed herein, an extracellular domain of a transmembrane protein, with or without the signal peptide, as disclosed herein or any other specifically defined fragment of the full-length amino acid sequence as disclosed herein, or (b) the complement of the DNA molecule of (a).

In other aspects, the isolated nucleic acid molecule comprises a nucleotide sequence having at least about 80% nucleic acid sequence identity, alternatively at least about 81% nucleic acid sequence identity, alternatively at least about 82% nucleic acid sequence identity, alternatively at least about 83% nucleic acid sequence identity, alternatively at least about 84% nucleic acid sequence identity, alternatively at least about 85% nucleic acid sequence identity, alternatively at least about 86% nucleic acid sequence identity, alternatively at least about 87% nucleic acid sequence identity, alternatively at least about 88% nucleic acid sequence identity, alternatively at least about 89% nucleic acid sequence identity, alternatively at least about 90% nucleic acid sequence identity, alternatively at least about 91% nucleic acid sequence identity, alternatively at least about 92% nucleic acid sequence identity, alternatively at least about 93% nucleic acid sequence identity, alternatively at least about 94% nucleic acid sequence identity, alternatively at least about 95% nucleic acid sequence identity, alternatively at least about 96% nucleic acid sequence identity, alternatively at least about 97% nucleic acid sequence identity, alternatively at least about 98% nucleic acid sequence identity and alternatively at least about 99% nucleic acid sequence identity to (a) a DNA molecule comprising the coding sequence of a full-length PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide cDNA as disclosed herein, the coding sequence of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide lacking the signal peptide as disclosed herein, the coding sequence of an extracellular domain of a transmembrane PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, with or without the signal peptide, as disclosed herein or the coding sequence of any other specifically defined fragment of the full-length amino acid sequence as disclosed herein, or (b) the complement of the DNA molecule of (a).

In a further aspect, the invention concerns an isolated nucleic acid molecule comprising a nucleotide sequence having at least about 80% nucleic acid sequence identity, alternatively at least about 81% nucleic acid sequence identity, alternatively at least about 82% nucleic acid sequence identity, alternatively at least about 83% nucleic acid sequence identity, alternatively at least about 84% nucleic acid sequence identity, alternatively at least about 85% nucleic acid sequence identity, alternatively at least about 86% nucleic acid sequence identity, alternatively at least about 87% nucleic acid sequence identity, alternatively at least about 88% nucleic acid sequence identity, alternatively at least about 89% nucleic acid sequence identity, alternatively at least about 90% nucleic acid sequence identity, alternatively at least about 91% nucleic acid sequence identity, alternatively at least about 92% nucleic acid sequence identity, alternatively at least about 93% nucleic acid sequence identity, alternatively at least about 94% nucleic acid sequence identity, alternatively at least about 95% nucleic acid sequence identity, alternatively at least about 96% nucleic acid sequence identity, alternatively at least about 97% nucleic acid sequence identity, alternatively at least about 98% nucleic acid sequence identity and alternatively at least about 99% nucleic acid sequence identity to (a) a DNA molecule that encodes the same mature polypeptide encoded by any of the human protein cDNAs deposited with the ATCC as disclosed herein, or (b) the complement of the DNA molecule of (a).

Another aspect of the invention provides an isolated nucleic acid molecule comprising a nucleotide sequence encoding a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide which is either transmembrane domain-deleted or transmembrane domain-inactivated, or is complementary to such encoding nucleotide sequence, wherein the transmembrane domain(s) of such polypeptide are disclosed herein. Therefore, soluble extracellular domains of the herein described PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides are contemplated.

The invention also provides fragments of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide coding sequence, or the complement thereof, that may find use as, for example, hybridization probes, for encoding fragments of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide that may optionally encode a polypeptide comprising a binding site for an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO 1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody or as antisense oligonucleotide probes. Such nucleic acid fragments usually are or are at least about 10 nucleotides in length, alternatively are or are at least about 15 nucleotides in length, alternatively are or are at least about 20 nucleotides in length, alternatively are or are at least about 30 nucleotides in length, alternatively are or are at least about 40 nucleotides in length, alternatively are or are at least about 50 nucleotides in length, alternatively are or are at least about 60 nucleotides in length, alternatively are or are at least about 70 nucleotides in length, alternatively are or are at least about 80 nucleotides in length, alternatively are or are at least about 90 nucleotides in length, alternatively are or are at least about 100 nucleotides in length, alternatively are or are at least about 110 nucleotides in length, alternatively are or are at least about 120 nucleotides in length, alternatively are or are at least about 130 nucleotides in length, alternatively are or are at least about 140 nucleotides in length, alternatively are or are at least about 150 nucleotides in length, alternatively are or are at least about 160 nucleotides in length, alternatively are or are at least about 170 nucleotides in length, alternatively are or are at least about 180 nucleotides in length, alternatively are or are at least about 190 nucleotides in length, alternatively are or are at least about 200 nucleotides in length, alternatively are or are at least about 250 nucleotides in length, alternatively are or are at least about 300 nucleotides in length, alternatively are or are at least about 350 nucleotides in length, alternatively are or are at least about 400 nucleotides in length, alternatively are or are at least about 450 nucleotides in length, alternatively are or are at least about 500 nucleotides in length, alternatively are or are at least about 600 nucleotides in length, alternatively are or are at least about 700 nucleotides in length, alternatively are or are at least about 800 nucleotides in length, alternatively are or are at least about 900 nucleotides in length and alternatively are or are at least about 1000 nucleotides in length, wherein in this context the term “about” means the referenced nucleotide sequence length plus or minus 10% of that referenced length. It is noted that novel fragments of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide-encoding nucleotide sequence may be determined in a routine manner by aligning the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide-encoding nucleotide sequence with other known nucleotide sequences using any of a number of well known sequence alignment programs and determining which PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide-encoding nucleotide sequence fragment(s) are novel. All of such PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide-encoding nucleotide sequences are contemplated herein. Also contemplated are the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide fragments encoded by these nucleotide molecule fragments, preferably those PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide fragments that comprise a binding site for an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody.

The invention provides isolated PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides encoded by any of the isolated nucleic acid sequences hereinabove identified.

In a certain aspect, the invention concerns an isolated PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, comprising an amino acid sequence having at least about 80% amino acid sequence identity, alternatively at least about 81% amino acid sequence identity, alternatively at least about 82% amino acid sequence identity, alternatively at least about 83% amino acid sequence identity, alternatively at least about 84% amino acid sequence identity, alternatively at least about 85% amino acid sequence identity, alternatively at least about 86% amino acid sequence identity, alternatively at least about 87% amino acid sequence identity, alternatively at least about 88% amino acid sequence identity, alternatively at least about 89% amino acid sequence identity, alternatively at least about 90% amino acid sequence identity, alternatively at least about 91% amino acid sequence identity, alternatively at least about 92% amino acid sequence identity, alternatively at least about 93% amino acid sequence identity, alternatively at least about 94% amino acid sequence identity, alternatively at least about 95% amino acid sequence identity, alternatively at least about 96% amino acid sequence identity, alternatively at least about 97% amino acid sequence identity, alternatively at least about 98% amino acid sequence identity and alternatively at least about 99% amino acid sequence identity to a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide having a full-length amino acid sequence as disclosed herein, an amino acid sequence lacking the signal peptide as disclosed herein, an extracellular domain of a transmembrane protein, with or without the signal peptide, as disclosed herein or any other specifically defined fragment of the full-length amino acid sequence as disclosed herein.

In a further aspect, the invention concerns an isolated PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide comprising an amino acid sequence having at least about 80% amino acid sequence identity, alternatively at least about 81% amino acid sequence identity, alternatively at least about 82% amino acid sequence identity, alternatively at least about 83% amino acid sequence identity, alternatively at least about 84% amino acid sequence identity, alternatively at least about 85% amino acid sequence identity, alternatively at least about 86% amino acid sequence identity, alternatively at least about 87% amino acid sequence identity, alternatively at least about 88% amino acid sequence identity, alternatively at least about 89% amino acid sequence identity, alternatively at least about 90% amino acid sequence identity, alternatively at least about 91% amino acid sequence identity, alternatively at least about 92% amino acid sequence identity, alternatively at least about 93% amino acid sequence identity, alternatively at least about 94% amino acid sequence identity, alternatively at least about 95% amino acid sequence identity, alternatively at least about 96% amino acid sequence identity, alternatively at least about 97% amino acid sequence identity, alternatively at least about 98% amino acid sequence identity and alternatively at least about 99% amino acid sequence identity to an amino acid sequence encoded by any of the human protein cDNAs deposited with the ATCC as disclosed herein.

In one aspect, the invention concerns PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 variant polypeptides which are or are at least about 10 amino acids in length, alternatively are or are at least about 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600 amino acids in length, or more. Optionally, PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 variant polypeptides will have or have no more than one conservative amino acid substitution as compared to the native PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide sequence, alternatively will have or will have no more than 2, 3, 4, 5, 6, 7, 8, 9, or 10 conservative amino acid substitution as compared to the native PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide sequence.

In a specific aspect, the invention provides an isolated PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide without the N-terminal signal sequence and/or the initiating methionine and is encoded by a nucleotide sequence that encodes such an amino acid sequence as hereinbefore described. Processes for producing the same are also herein described, wherein those processes comprise culturing a host cell comprising a vector which comprises the appropriate encoding nucleic acid molecule under conditions suitable for expression of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide and recovering the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide from the cell culture.

Another aspect the invention provides an isolated PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide which is either transmembrane domain-deleted or transmembrane domain-inactivated. Processes for producing the same are also herein described, wherein those processes comprise culturing a host cell comprising a vector which comprises the appropriate encoding nucleic acid molecule under conditions suitable for expression of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide and recovering the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide from the cell culture.

The invention provides agonists and antagonists of a native PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide as defined herein. In particular, the agonist or antagonist is an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody or a small molecule.

The invention provides a method of identifying agonists or antagonists to a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide which comprise contacting the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide with a candidate molecule and monitoring a biological activity mediated by said PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide. Preferably, the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide is a native PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide.

The invention provides a composition of matter comprising a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, or an agonist or antagonist of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide as herein described, or an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody, in combination with a carrier. Optionally, the carrier is a pharmaceutically acceptable carrier.

The invention provides the use of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, or an agonist or antagonist thereof as hereinbefore described, or an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody, for the preparation of a medicament useful in the treatment of a condition which is responsive to the anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody.

The invention provides vectors comprising DNA encoding any of the herein described polypeptides. Host cell comprising any such vector are also provided. By way of example, the host cells may be CHO cells, E. coli, or yeast. A process for producing any of the herein described polypeptides is further provided and comprises culturing host cells under conditions suitable for expression of the desired polypeptide and recovering the desired polypeptide from the cell culture.

The invention provides chimeric molecules comprising any of the herein described polypeptides fused to a heterologous polypeptide or amino acid sequence. Example of such chimeric molecules comprise any of the herein described polypeptides fused to an epitope tag sequence or a Fc region of an immunoglobulin.

The invention provides an antibody which binds, preferably specifically, to any of the above or below described polypeptides. Optionally, the antibody is a monoclonal antibody, humanized antibody, antibody fragment or single-chain antibody.

The invention provides oligonucleotide probes which may be useful for isolating genomic and cDNA nucleotide sequences, measuring or detecting expression of an associated gene or as antisense probes, wherein those probes may be derived from any of the above or below described nucleotide sequences. Preferred probe lengths are described above.

The invention also provides a method of identifying a phenotype associated with a disruption of a gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising:

(a) providing a non-human transgenic animal whose genome comprises a disruption of the gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide;

(b) measuring a physiological characteristic of the non-human transgenic animal; and

(c) comparing the measured physiological characteristic with that of a gender matched wild-type animal, wherein the physiological characteristic of the non-human transgenic animal that differs from the physiological characteristic of the wild-type animal is identified as a phenotype resulting from the gene disruption in the non-human transgenic animal. In one aspect, the non-human transgenic animal is a mammal. In another aspect, the mammal is a rodent. In still another aspect, the mammal is a rat or a mouse. In one aspect, the non-human transgenic animal is heterozygous for the disruption of a gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide. In another aspect, the phenotype exhibited by the non-human transgenic animal as compared with gender matched wild-type littermates is at least one of the following: a neurological disorder; a cardiovascular, endothelial or angiogenic disorder; an eye abnormality; an immunological disorder; an oncological disorder; a bone metabolic abnormality or disorder; a lipid metabolic disorder; or a developmental abnormality.

In yet another aspect, the neurological disorder is an increased anxiety-like response during open field activity testing. In yet another aspect, the neurological disorder is a decreased anxiety-like response during open field activity testing. In yet another aspect, the neurological disorder is an abnormal circadian rhythm during home-cage activity testing. In yet another aspect, the neurological disorder is an enhanced motor coordination during inverted screen testing. In yet another aspect, the neurological disorder is impaired motor coordination during inverted screen testing. In yet another aspect, the neurological disorder includes depression, generalized anxiety disorders, attention deficit disorder, sleep disorder, hyperactivity disorder, obsessive compulsive disorder, schizophrenia, cognitive disorders, hyperalgesia and sensory disorders. Such neurological disorders include the category defined as “anxiety disorders” which include but are not limited to: mild to moderate anxiety, anxiety disorder due to a general medical condition, anxiety disorder not otherwise specified, generalized anxiety disorder, panic attack, panic disorder with agoraphobia, panic disorder without agoraphobia, posttraumatic stress disorder, social phobia, social anxiety, autism, specific phobia, substance-induced anxiety disorder, acute alcohol withdrawal, obsessive compulsive disorder, agoraphobia, monopolar disorders, bipolar disorder I or II, bipolar disorder not otherwise specified, cyclothymic disorder, depressive disorder, major depressive disorder, mood disorder, substance-induced mood disorder, enhancement of cognitive function, loss of cognitive function associated with but not limited to Alzheimer's disease, stroke, or traumatic injury to the brain, seizures resulting from disease or injury including but not limited to epilepsy, learning disorders/disabilities, cerebral palsy. In addition, anxiety disorders may apply to personality disorders including but not limited to the following types: paranoid, antisocial, avoidant behavior, borderline personality disorders, dependent, histronic, narcissistic, obsessive-compulsive, schizoid, and schizotypal.

In another aspect, the eye abnormality is a retinal abnormality. In still another aspect, the eye abnormality is consistent with vision problems or blindness. In yet another aspect, the retinal abnormality is consistent with retinitis pigmentosa or is characterized by retinal degeneration or retinal dysplasia.

In still another aspect, the retinal abnormalities are consistent with retinal dysplasia, various retinopathies, including retinopathy of prematurity, retrolental fibroplasia, neovascular glaucoma, age-related macular degeneration, diabetic macular edema, corneal neovascularization, corneal graft neovascularization, corneal graft rejection, retinal/choroidal neovascularization, neovascularization of the angle (rubeosis), ocular neovascular disease, vascular restenosis, arteriovenous malformations (AVM), meningioma, hemangioma, angiofibroma, thyroid hyperplasias (including Grave's disease), corneal and other tissue transplantation, retinal artery obstruction or occlusion; retinal degeneration causing secondary atrophy of the retinal vasculature, retinitis pigmentosa, macular dystrophies, Stargardt's disease, congenital stationary night blindness, choroideremia, gyrate atrophy, Leber's congenital amaurosis, retinoschisis disorders, Wagner's syndrome, Usher syndromes, Zellweger syndrome, Saldino-Mainzer syndrome, Senior-Loken syndrome, Bardet-Biedl syndrome, Alport's syndrome, Alstrom's syndrome, Cockayne's syndrome, dysplaisa spondyloepiphysaria congentia, Flynn-Aird syndrome, Friedreich ataxia, Hallgren syndrome, Marshall syndrome, Albers-Schnoberg disease, Refsum's disease, Kearns-Sayre syndrome, Waardenburg's syndrome, Alagile syndrome, myotonic dystrophy, olivopontocerebellar atrophy, Pierre-Marie dunsdrome, Stickler syndrome, carotinemeia, cystinosis, Wolfram syndrome, Bassen-Kornzweig syndrome, abetalipoproteinemia, incontinentiapigmenti, Batten's disease, mucopolysaccharidoses, homocystinuria, or mannosidosis.

In still another aspect, the eye abnormality is a cataract. In still yet another aspect, the cataract is a systemic disease such as human Down's syndrome, Hallerman-Streiff syndrome, Lowe syndrome, galactosemia, Marfan syndrome, Trismoy 13-15, Alport syndrome, myotonic dystrophy, Fabry disease, hypoparathroidism or Conradi syndrome.

In still another aspect, the developmental abnormality comprises embryonic lethality or reduced viability.

In still yet another aspect, the cardiovascular, endothelial or angiogenic disorders are arterial diseases, such as diabetes mellitus; papilledema; optic atrophy; atherosclerosis; angina; myocardial infarctions such as acute myocardial infarctions, cardiac hypertrophy, and heart failure such as congestive heart failure; hypertension; inflammatory vasculitides; Reynaud's disease and Reynaud's phenomenon; aneurysms and arterial restenosis; venous and lymphatic disorders such as thrombophlebitis, lymphangitis, and lymphedema; peripheral vascular disease; cancer such as vascular tumors, e.g., hemangioma (capillary and cavernous), glomus tumors, telangiectasia, bacillary angiomatosis, hemangioendothelioma, angiosarcoma, haemangiopericytoma, Kaposi's sarcoma, lymphangioma, and lymphangiosarcoma; tumor angiogenesis; trauma such as wounds, burns, and other injured tissue, implant fixation, scarring; ischemia reperfusion injury; rheumatoid arthritis; cerebrovascular disease; renal diseases such as acute renal failure, or osteoporosis.

In still another aspect, the immunological disorders are consistent with systemic lupus erythematosis; rheumatoid arthritis; juvenile chronic arthritis; spondyloarthropathies; systemic sclerosis (scleroderma); idiopathic inflammatory myopathies (dermatomyositis, polymyositis); Sjögren's syndrome; systemic vasculitis; sarcoidosis; autoimmune hemolytic anemia (immune pancytopenia, paroxysmal nocturnal hemoglobinuria); autoimmune thrombocytopenia (idiopathic thrombocytopenic purpura, immune-mediated thrombocytopenia); thyroiditis (Grave's disease, Hashimoto's thyroiditis, juvenile lymphocytic thyroiditis, atrophic thyroiditis); diabetes mellitus; immune-mediated renal disease (glomerulonephritis, tubulointerstitial nephritis); demyelinating diseases of the central and peripheral nervous systems such as multiple sclerosis, idiopathic demyelinating polyneuropathy or Guillain-Barré syndrome, and chronic inflammatory demyelinating polyneuropathy; hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis; inflammatory bowel disease (ulcerative colitis: Crohn's disease); gluten-sensitive enteropathy, and Whipple's disease; autoimmune or immune-mediated skin diseases including bullous skin diseases, erythema multiforme and contact dermatitis, psoriasis; allergic diseases such as asthma, allergic rhinitis, atopic dermatitis, food hypersensitivity and urticaria; immunologic diseases of the lung such as eosinophilic pneumonia, idiopathic pulmonary fibrosis and hypersensitivity pneumonitis; or transplantation associated diseases including graft rejection and graft-versus-host disease.

In still another aspect, the bone metabolic abnormality or disorder is arthritis, osteoporosis, osteopenia or osteopetrosis.

In another aspect, the non-human transgenic animal exhibits at least one of the following physiological characteristics compared with gender matched wild-type littermates: increased anxiety-like response during open field testing; decreased anxiety-like response during open field activity testing; hyperactivity with increased rearing and hole poke activity during open field testing; hypoactivity with decreased rearing and hole poke activity during open field testing; increased exploratory activity during open-field testing; decreased exploratory activity during open-field testing; augmentation of circadian rhythm; abnormal circadian rhythm during home-cage activity testing including decreased ambulatory counts; abnormal circadian rhythm during home-cage activity testing including increased ambulatory counts; enhanced circadian rhythm; increased stress induced hyperthermia with increased stress response; increased resistance to stress induced hyperthermia; decreased resistance to stress induced hyperthermia; impaired motor coordination during inverted screen testing; increased depressive-like response during tail suspension testing; decreased depressive-like response during tail suspension testing; decreased startle response during prepulse inhibition testing; no startle response indicating deafness; reduced latency to respond in hot plate testing; increased pain perception in hot plate testing; prolonged latency to respond in hot plate testing; decreased pain perception in hot plate testing; straub tails during functional observational battery testing; opthamological abnormalities; attenuated retinal arteries; optic nerve abnormalities; retinal degeneration; retinal depigmentation; cataracts; decreased heart rate; decreased mean systolic blood pressure; increased mean systolic blood pressure; increased insulin sensitivity; increased mean fasting serum glucose levels; decreased mean serum glucose levels; increased mean serum cholesterol levels; decreased mean serum cholesterol levels; increased mean serum triglyceride levels; decreased mean serum triglyceride levels; enhanced glucose tolerance; impaired glucose tolerance; decreased mean serum insulin levels; increased uric acid levels; ketonemia; increased mean serum phosphorous levels; increased mean serum potassium levels; increased mean serum alkaline phosphatase levels; decreased mean serum alkaline phosphatase levels; blood in the urine; increased nitrituria; ketonuria; decreased mean serum albumin; decreased mean percentage of natural killer cells; abnormal leukocyte count; increased mean percentage of CD4 cells; decreased mean percentage of CD4 cells; increased mean percentage of B cells in peripheral blood; increase in CD4+ and CD8+ cells with decrease in B cells; decreased B cells and less CD11 blow cells in peritoneum; increased mean percentage B cells in spleen, lymph nodes and Peyer's patches; increase in activated/memory T cells by CD25+ staining and CD62L/CD44 staining; increase in activated/memory T cells in spleen; decreased mean percentage of CD8+ cells; increase total white blood cells (increase in neutrophils, lymphocytes, monocytes and basophils); decreased lymphocytes; increased mean absolute monocyte count; increased mean absolute neutrophil count; decreased mean absolute monocyte count; decreased mean serum IgM, IgA, IgG3, IgG2b and IgG2a levels; decreased mean serum IgG3 levels; decreased mean serum IgM levels; decreased mean serum IgG2a levels; decreased mean serum IgG3 and IgM levels; increase in mean serum IgM levels; increase in mean serum IgG2a levels; increase in mean serum IgG2b levels; anemia; decreased red blood cell count, decreased hemoglobin and decreased hematocrit; increased mean corpuscular volume; increased mean corpuscular hemoglobin; decreased mean corpuscular volume; decreased mean corpuscular hemoglobin; increased red blood cell distribution width and mean platelet volume; decreased red blood cell distribution width; skewed ratios of B220med/CD23− and B220+/CD11− low/CD23− cells after peritoneal lavage; increased CD25 T cells in lymph node and spleen; increased CD38 non-lymphoid cells in Peyer's patches; increased CD23 B cells (peritoneal); decreased percentage of CD4/CD8 DP cells and increased percentage of TCRB+ cells in thymus; decrease in Peyer's patch B cells; reduced number of TCRB+ CD38+ activated T cells in Peyer's patches; increased splenic CD25+ cells and peritoneal CD23 B cells; increased mean platelet count; decreased mean platelet count; decreased mean serum IgG1 response to an ovalbumin challenge; decreased mean serum IgG2a response to an ovalbumin challenge; increased mean serum IgG2a response to an ovalbumin challenge; increased mean serum MCP-1 response to a LPS challenge; increased mean serum TNF-alpha response to a LPS challenge; increased mean serum IL-6 response to a LPS challenge; increased skin fibroblast proliferation; decreased skin fibroblast proliferation; increased mean percent of total body fat and total fat mass; increased mean body weight; increased mean body length; increased total tissue mass (TTM); increased lean body mass (LBM); increased femoral bone mineral density (BMD); increased vertebral bone mineral density (BMD); increased BMC/LBM ratio; increased bone mineral density (BMD); increased total body volumetric bone mineral density (vBMD); increased bone mineral content (BMC); increased mean femoral midshaft cortical thickness and cross-sectional area; increased mean vertebral trabecular bone volume, number and connectivity density; decreased mean percent of total body fat and total fat mass; decreased mean body weight; decreased mean body length; decreased total tissue mass (TTM); decreased lean body mass (LBM); decreased femoral bone mineral density (BMD); decreased vertebral bone mineral density (BMD); decreased BMC/LBM ratio; decreased bone mineral density (BMD); decreased bone mineral content (BMC); decreased volumetric bone mineral density (vBMD); decreased mean femoral midshaft cortical thickness and cross-sectional area; decreased mean vertebral trabecular bone volume, number and connectivity density; myeloid hyperplasia in bone marrow; osteopetrosis with increased bone mineralization; increase in abdominal fat depots; chronic-active arthritis; proliferative chondrapathy and arthropathy; proliferation of cartilage in femoral tibia joints; chondrous metaplasia of cruciate ligaments and perichondral connective tissues; chronic active dermatitis; chronic active inflammation in periarticular tissues; chronic inflammation in various tissues; myeloid hyperplasia in femur and sternum with associated erythroid hyperplasia in the spleen; increased spleen weight; impaired gastrointestinal motility; thymic atrophy; thymic T cell lymphoma; growth retardation; development abnormalities; stunted growth with general reduction in all organ size; growth retardation with reduced viability; and embryonic lethality.

The invention also provides an isolated cell derived from a non-human transgenic animal whose genome comprises a disruption of the gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide. In one aspect, the isolated cell is a murine cell. In yet another aspect, the murine cell is an embryonic stem cell. In still another aspect, the isolated cell is derived from a non-human transgenic animal which exhibits at least one of the following phenotypes compared with gender matched wild-type littermates: a neurological disorder; a cardiovascular, endothelial or angiogenic disorder; an eye abnormality; an immunological disorder; an oncological disorder; a bone metabolic abnormality or disorder; a lipid metabolic disorder; or a developmental abnormality. The invention also provides a method of identifying an agent that modulates a phenotype associated with a disruption of a gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising:

(a) providing a non-human transgenic animal whose genome comprises a disruption of the gene which encodes for the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide;

(b) measuring a physiological characteristic of the non-human transgenic animal of (a);

(c) comparing the measured physiological characteristic of (b) with that of a gender matched wild-type animal, wherein the physiological characteristic of the non-human transgenic animal that differs from the physiological characteristic of the wild-type animal is identified as a phenotype resulting from the gene disruption in the non-human transgenic animal;

(d) administering a test agent to the non-human transgenic animal of (a); and

(e) determining whether the test agent modulates the identified phenotype associated with gene disruption in the non-human transgenic animal.

In one aspect, the phenotype associated with the gene disruption comprises a neurological disorder; a cardiovascular, endothelial or angiogenic disorder; an eye abnormality; an immunological disorder; an oncological disorder; a bone metabolic abnormality or disorder; a lipid metabolic disorder; or a developmental abnormality.

In yet another aspect, the neurological disorder is an increased anxiety-like response during open field activity testing. In yet another aspect, the neurological disorder is a decreased anxiety-like response during open field activity testing. In yet another aspect, the neurological disorder is an abnormal circadian rhythm during home-cage activity testing. In yet another aspect, the neurological disorder is an enhanced motor coordination during inverted screen testing. In yet another aspect, the neurological disorder is impaired motor coordination during inverted screen testing. In yet another aspect, the neurological disorder includes depression, generalized anxiety disorders, attention deficit disorder, sleep disorder, hyperactivity disorder, obsessive compulsive disorder, schizophrenia, cognitive disorders, hyperalgesia and sensory disorders. Such neurological disorders include the category defined as “anxiety disorders” which include but are not limited to: mild to moderate anxiety, anxiety disorder due to a general medical condition, anxiety disorder not otherwise specified, generalized anxiety disorder, panic attack, panic disorder with agoraphobia, panic disorder without agoraphobia, posttraumatic stress disorder, social phobia, social anxiety, autism, specific phobia, substance-induced anxiety disorder, acute alcohol withdrawal, obsessive compulsive disorder, agoraphobia, monopolar disorders, bipolar disorder I or II, bipolar disorder not otherwise specified, cyclothymic disorder, depressive disorder, major depressive disorder, mood disorder, substance-induced mood disorder, enhancement of cognitive function, loss of cognitive function associated with but not limited to Alzheimer's disease, stroke, or traumatic injury to the brain, seizures resulting from disease or injury including but not limited to epilepsy, learning disorders/disabilities, cerebral palsy. In addition, anxiety disorders may apply to personality disorders including but not limited to the following types: paranoid, antisocial, avoidant behavior, borderline personality disorders, dependent, histronic, narcissistic, obsessive-compulsive, schizoid, and schizotypal.

In yet another aspect, the eye abnormality is a retinal abnormality. In still another aspect, the eye abnormality is consistent with vision problems or blindness. In yet another aspect, the retinal abnormality is consistent with retinitis pigmentosa or is characterized by retinal degeneration or retinal dysplasia.

In still another aspect, the retinal abnormalities are consistent with retinal dysplasia, various retinopathies, including retinopathy of prematurity, retrolental fibroplasia, neovascular glaucoma, age-related macular degeneration, diabetic macular edema, corneal neovascularization, corneal graft neovascularization, corneal graft rejection, retinal/choroidal neovascularization, neovascularization of the angle (rubeosis), ocular neovascular disease, vascular restenosis, arteriovenous malformations (AVM), meningioma, hemangioma, angiofibroma, thyroid hyperplasias (including Grave's disease), corneal and other tissue transplantation, retinal artery obstruction or occlusion; retinal degeneration causing secondary atrophy of the retinal vasculature, retinitis pigmentosa, macular dystrophies, Stargardt's disease, congenital stationary night blindness, choroideremia, gyrate atrophy, Leber's congenital amaurosis, retinoschisis disorders, Wagner's syndrome, Usher syndromes, Zellweger syndrome, Saldino-Mainzer syndrome, Senior-Loken syndrome, Bardet-Biedl syndrome, Alport's syndrome, Alstrom's syndrome, Cockayne's syndrome, dysplaisa spondyloepiphysaria congentia, Flynn-Aird syndrome, Friedreich ataxia, Hallgren syndrome, Marshall syndrome, Albers-Schnoberg disease, Refsum's disease, Kearns-Sayre syndrome, Waardenburg's syndrome, Alagile syndrome, myotonic dystrophy, olivopontocerebellar atrophy, Pierre-Marie dunsdrome, Stickler syndrome, carotinemeia, cystinosis, Wolfram syndrome, Bassen-Kornzweig syndrome, abetalipoproteinemia, incontinentia pigmenti, Batten's disease, mucopolysaccharidoses, homocystinuria, or mannosidosis.

In still another aspect, the eye abnormality is a cataract. In still yet another aspect, the cataract is a systemic disease such as human Down's syndrome, Hallerman-Streiff syndrome, Lowe syndrome, galactosemia, Marfan syndrome, Trismoy 13-15, Alport syndrome, myotonic dystrophy, Fabry disease, hypoparathroidism, or Conradi syndrome.

In still another aspect, the developmental abnormality comprises embryonic lethality or reduced viability.

In still another aspect, the cardiovascular, endothelial or angiogenic disorders are arterial diseases, such as diabetes mellitus; papilledema; optic atrophy; atherosclerosis; angina; myocardial infarctions such as acute myocardial infarctions, cardiac hypertrophy, and heart failure such as congestive heart failure; hypertension; inflammatory vasculitides; Reynaud's disease and Reynaud's phenomenon; aneurysms and arterial restenosis; venous and lymphatic disorders such as thrombophlebitis, lymphangitis, and lymphedema; peripheral vascular disease; cancer such as vascular tumors, e.g., hemangioma (capillary and cavernous), glomus tumors, telangiectasia, bacillary angiomatosis, hemangioendothelioma, angiosarcoma, haemangiopericytoma, Kaposi's sarcoma, lymphangioma, and lymphangiosarcoma; tumor angiogenesis; trauma such as wounds, burns, and other injured tissue, implant fixation, scarring; ischemiareperfusion injury; rheumatoid arthritis; cerebrovascular disease; renal diseases such as acute renal failure, or osteoporosis.

In still another aspect, the immunological disorders are consistent with systemic lupus erythematosis; rheumatoid arthritis; juvenile chronic arthritis; spondyloarthropathies; systemic sclerosis (scleroderma); idiopathic inflammatory myopathies (dermatomyositis, polymyositis); Sjögren's syndrome; systemic vasculitis; sarcoidosis; autoimmune hemolytic anemia (immune pancytopenia, paroxysmal nocturnal hemoglobinuria); autoimmune thrombocytopenia (idiopathic thrombocytopenic purpura, immune-mediated thrombocytopenia); thyroiditis (Grave's disease, Hashimoto's thyroiditis, juvenile lymphocytic thyroiditis, atrophic thyroiditis); diabetes mellitus; immune-mediated renal disease (glomerulonephritis, tubulointerstitial nephritis); demyelinating diseases of the central and peripheral nervous systems such as multiple sclerosis, idiopathic demyelinating polyneuropathy or Guillain-Barré syndrome, and chronic inflammatory demyelinating polyneuropathy; hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis; inflammatory bowel disease (ulcerative colitis: Crohn's disease); gluten-sensitive enteropathy, and Whipple's disease; autoimmune or immune-mediated skin diseases including bullous skin diseases, erythema multiforme and contact dermatitis, psoriasis; allergic diseases such as asthma, allergic rhinitis, atopic dermatitis, food hypersensitivity and urticaria; immunologic diseases of the lung such as eosinophilic pneumonia, idiopathic pulmonary fibrosis and hypersensitivity pneumonitis; or transplantation associated diseases including graft rejection and graft-versus-host disease.

In yet another aspect, the bone metabolic abnormality or disorder is arthritis, osteoporosis, osteopenia or osteopetrosis.

In another aspect, the non-human transgenic animal exhibits at least one of the following physiological characteristics compared with gender matched wild-type littermates: increased anxiety-like response during open field testing; decreased anxiety-like response during open field activity testing; hyperactivity with increased rearing and hole poke activity during open field testing; hypoactivity with decreased rearing and hole poke activity during open field testing; increased exploratory activity during open-field testing; decreased exploratory activity during open-field testing; augmentation of circadian rhythm; abnormal circadian rhythm during home-cage activity testing including decreased ambulatory counts; abnormal circadian rhythm during home-cage activity testing including increased ambulatory counts; enhanced circadian rhythm; increased stress induced hyperthermia with increased stress response; increased resistance to stress induced hyperthermia; decreased resistance to stress induced hyperthermia; impaired motor coordination during inverted screen testing; increased depressive-like response during tail suspension testing; decreased depressive-like response during tail suspension testing; decreased startle response during prepulse inhibition testing; no startle response indicating deafness; reduced latency to respond in hot plate testing; increased pain perception in hot plate testing; prolonged latency to respond in hot plate testing; decreased pain perception in hot plate testing; straub tails during functional observational battery testing; opthamological abnormalities; attenuated retinal arteries; optic nerve abnormalities; retinal degeneration; retinal depigmentation; cataracts; decreased heart rate; decreased mean systolic blood pressure; increased mean systolic blood pressure; increased insulin sensitivity; increased mean fasting serum glucose levels; decreased mean serum glucose levels; increased mean serum cholesterol levels; decreased mean serum cholesterol levels; increased mean serum triglyceride levels; decreased mean serum triglyceride levels; enhanced glucose tolerance; impaired glucose tolerance; decreased mean serum insulin levels; increased uric acid levels; ketonemia; increased mean serum phosphorous levels; increased mean serum potassium levels; increased mean serum alkaline phosphatase levels; decreased mean serum alkaline phosphatase levels; blood in the urine; increased nitrituria; ketonuria; decreased mean serum albumin; decreased mean percentage of natural killer cells; abnormal leukocyte count; increased mean percentage of CD4 cells; decreased mean percentage of CD4 cells; increased mean percentage of B cells in peripheral blood; increase in CD4+ and CD8+ cells with decrease in B cells; decreased B cells and less CD11 blow cells in peritoneum; increased mean percentage B cells in spleen, lymph nodes and Peyer's patches; increase in activated/memory T cells by CD25+ staining and CD62L/CD44 staining; increase in activated/memory T cells in spleen; decreased mean percentage of CD8+ cells; increase total white blood cells (increase in neutrophils, lymphocytes, monocytes and basophils); decreased lymphocytes; increased mean absolute monocyte count; increased mean absolute neutrophil count; decreased mean absolute monocyte count; decreased mean serum IgM, IgA, IgG3, IgG2b and IgG2a levels; decreased mean serum IgG3 levels; decreased mean serum IgM levels; decreased mean serum IgG2a levels; decreased mean serum IgG3 and IgM levels; increase in mean serum IgM levels; increase in mean serum IgG2a levels; increase in mean serum IgG2b levels; anemia; decreased red blood cell count, decreased hemoglobin and decreased hematocrit; increased mean corpuscular volume; increased mean corpuscular hemoglobin; decreased mean corpuscular volume; decreased mean corpuscular hemoglobin; increased red blood cell distribution width and mean platelet volume; decreased red blood cell distribution width; skewed ratios of B220med/CD23− and B220+/CD11− low/CD23− cells after peritoneal lavage; increased CD25 T cells in lymph node and spleen; increased CD38 non-lymphoid cells in Peyer's patches; increased CD23 B cells (peritoneal); decreased percentage of CD4/CD8 DP cells and increased percentage of TCRB+ cells in thymus; decrease in Peyer's patch B cells; reduced number of TCRB+ CD38+ activated T cells in Peyer's patches; increased splenic CD25+ cells and peritoneal CD23 B cells; increased mean platelet count; decreased mean platelet count; decreased mean serum IgG1 response to an ovalbumin challenge; decreased mean serum IgG2a response to an ovalbumin challenge; increased mean serum IgG2a response to an ovalbumin challenge; increased mean serum MCP-1 response to a LPS challenge; increased mean serum TNF-alpha response to a LPS challenge; increased mean serum IL-6 response to a LPS challenge; increased skin fibroblast proliferation; decreased skin fibroblast proliferation; increased mean percent of total body fat and total fat mass; increased mean body weight; increased mean body length; increased total tissue mass (TTM); increased lean body mass (LBM); increased femoral bone mineral density (BMD); increased vertebral bone mineral density (BMD); increased BMC/LBM ratio; increased bone mineral density (BMD); increased total body volumetric bone mineral density (vBMD); increased bone mineral content (BMC); increased mean femoral midshaft cortical thickness and cross-sectional area; increased mean vertebral trabecular bone volume, number and connectivity density; decreased mean percent of total body fat and total fat mass; decreased mean body weight; decreased mean body length; decreased total tissue mass (TTM); decreased lean body mass (LBM); decreased femoral bone mineral density (BMD); decreased vertebral bone mineral density (BMD); decreased BMC/LBM ratio; decreased bone mineral density (BMD); decreased bone mineral content (BMC); decreased volumetric bone mineral density (vBMD); decreased mean femoral midshaft cortical thickness and cross-sectional area; decreased mean vertebral trabecular bone volume, number and connectivity density; myeloid hyperplasia in bone marrow; osteopetrosis with increased bone mineralization; increase in abdominal fat depots; chronic-active arthritis; proliferative chondrapathy and arthropathy; proliferation of cartilage in femoral tibia joints; chondrous metaplasia of cruciate ligaments and perichondral connective tissues; chronic active dermatitis; chronic active inflammation in periarticular tissues; chronic inflammation in various tissues; myeloid hyperplasia in femur and sternum with associated erythroid hyperplasia in the spleen; increased spleen weight; impaired gastrointestinal motility; thymic atrophy; thymic T cell lymphoma; growth retardation; development abnormalities; stunted growth with general reduction in all organ size; growth retardation with reduced viability; and embryonic lethality.

The invention also provides an agent which modulates the phenotype associated with gene disruption. In one aspect, the agent is an agonist or antagonist of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide.

In yet another aspect, the agonist agent is an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody. In still another aspect, the antagonist agent is an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody.

The invention also provides a method of identifying an agent that modulates a physiological characteristic associated with a disruption of the gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising:

(a) providing a non-human transgenic animal whose genome comprises a disruption of the gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide;

(b) measuring a physiological characteristic exhibited by the non-human transgenic animal of (a);

(c) comparing the measured physiological characteristic of (b) with that of a gender matched wild-type animal, wherein the physiological characteristic exhibited by the non-human transgenic animal that differs from the physiological characteristic exhibited by the wild-type animal is identified as a physiological characteristic associated with gene disruption;

(d) administering a test agent to the non-human transgenic animal of (a); and

(e) determining whether the physiological characteristic associated with gene disruption is modulated.

In one aspect, the non-human transgenic animal exhibits at least one of the following physiological characteristics compared with gender matched wild-type littermates:

In another aspect, the non-human transgenic animal exhibits at least one of the following physiological characteristics compared with gender matched wild-type littermates: increased anxiety-like response during open field testing; decreased anxiety-like response during open field activity testing; hyperactivity with increased rearing and hole poke activity during open field testing; hypoactivity with decreased rearing and hole poke activity during open field testing; increased exploratory activity during open-field testing; decreased exploratory activity during open-field testing; augmentation of circadian rhythm; abnormal circadian rhythm during home-cage activity testing including decreased ambulatory counts; abnormal circadian rhythm during home-cage activity testing including increased ambulatory counts; enhanced circadian rhythm; increased stress induced hyperthermia with increased stress response; increased resistance to stress induced hyperthermia; decreased resistance to stress induced hyperthermia; impaired motor coordination during inverted screen testing; increased depressive-like response during tail suspension testing; decreased depressive-like response during tail suspension testing; decreased startle response during prepulse inhibition testing; no startle response indicating deafness; reduced latency to respond in hot plate testing; increased pain perception in hot plate testing; prolonged latency to respond in hot plate testing; decreased pain perception in hot plate testing; straub tails during functional observational battery testing; opthamological abnormalities; attenuated retinal arteries; optic nerve abnormalities; retinal degeneration; retinal depigmentation; cataracts; decreased heart rate; decreased mean systolic blood pressure; increased mean systolic blood pressure; increased insulin sensitivity; increased mean fasting serum glucose levels; decreased mean serum glucose levels; increased mean serum cholesterol levels; decreased mean serum cholesterol levels; increased mean serum triglyceride levels; decreased mean serum triglyceride levels; enhanced glucose tolerance; impaired glucose tolerance; decreased mean serum insulin levels; increased uric acid levels; ketonemia; increased mean serum phosphorous levels; increased mean serum potassium levels; increased mean serum alkaline phosphatase levels; decreased mean serum alkaline phosphatase levels; blood in the urine; increased nitrituria; ketonuria; decreased mean serum albumin; decreased mean percentage of natural killer cells; abnormal leukocyte count; increased mean percentage of CD4 cells; decreased mean percentage of CD4 cells; increased mean percentage of B cells in peripheral blood; increase in CD4+ and CD8+ cells with decrease in B cells; decreased B cells and less CD11 blow cells in peritoneum; increased mean percentage B cells in spleen, lymph nodes and Peyer's patches; increase in activated/memory T cells by CD25+ staining and CD62L/CD44 staining; increase in activated/memory T cells in spleen; decreased mean percentage of CD8+ cells; increase total white blood cells (increase in neutrophils, lymphocytes, monocytes and basophils); decreased lymphocytes; increased mean absolute monocyte count; increased mean absolute neutrophil count; decreased mean absolute monocyte count; decreased mean serum IgM, IgA, IgG3, IgG2b and IgG2a levels; decreased mean serum IgG3 levels; decreased mean serum IgM levels; decreased mean serum IgG2a levels; decreased mean serum IgG3 and IgM levels; increase in mean serum IgM levels; increase in mean serum IgG2a levels; increase in mean serum IgG2b levels; anemia; decreased red blood cell count, decreased hemoglobin and decreased hematocrit; increased mean corpuscular volume; increased mean corpuscular hemoglobin; decreased mean corpuscular volume; decreased mean corpuscular hemoglobin; increased red blood cell distribution width and mean platelet volume; decreased red blood cell distribution width; skewed ratios of B220med/CD23− and B220+/CD11− low/CD23− cells after peritoneal lavage; increased CD25 T cells in lymph node and spleen; increased CD38 non-lymphoid cells in Peyer's patches; increased CD23 B cells (peritoneal); decreased percentage of CD4/CD8 DP cells and increased percentage of TCRB+ cells in thymus; decrease in Peyer's patch B cells; reduced number of TCRB+ CD38+ activated T cells in Peyer's patches; increased splenic CD25+ cells and peritoneal CD23 B cells; increased mean platelet count; decreased mean platelet count; decreased mean serum IgG1 response to an ovalbumin challenge; decreased mean serum IgG2a response to an ovalbumin challenge; increased mean serum IgG2a response to an ovalbumin challenge; increased mean serum MCP-1 response to a LPS challenge; increased mean serum TNF-alpha response to a LPS challenge; increased mean serum IL-6 response to a LPS challenge; increased skin fibroblast proliferation; decreased skin fibroblast proliferation; increased mean percent of total body fat and total fat mass; increased mean body weight; increased mean body length; increased total tissue mass (TTM); increased lean body mass (LBM); increased femoral bone mineral density (BMD); increased vertebral bone mineral density (BMD); increased BMC/LBM ratio; increased bone mineral density (BMD); increased total body volumetric bone mineral density (vBMD); increased bone mineral content (BMC); increased mean femoral midshaft cortical thickness and cross-sectional area; increased mean vertebral trabecular bone volume, number and connectivity density; decreased mean percent of total body fat and total fat mass; decreased mean body weight; decreased mean body length; decreased total tissue mass (TTM); decreased lean body mass (LBM); decreased femoral bone mineral density (BMD); decreased vertebral bone mineral density (BMD); decreased BMC/LBM ratio; decreased bone mineral density (BMD); decreased bone mineral content (BMC); decreased volumetric bone mineral density (vBMD); decreased mean femoral midshaft cortical thickness and cross-sectional area; decreased mean vertebral trabecular bone volume, number and connectivity density; myeloid hyperplasia in bone marrow; osteopetrosis with increased bone mineralization; increase in abdominal fat depots; chronic-active arthritis; proliferative chondrapathy and arthropathy; proliferation of cartilage in femoral tibia joints; chondrous metaplasia of cruciate ligaments and perichondral connective tissues; chronic active dermatitis; chronic active inflammation in periarticular tissues; chronic inflammation in various tissues; myeloid hyperplasia in femur and sternum with associated erythroid hyperplasia in the spleen; increased spleen weight; impaired gastrointestinal motility; thymic atrophy; thymic T cell lymphoma; growth retardation; development abnormalities; stunted growth with general reduction in all organ size; growth retardation with reduced viability; and embryonic lethality.

The invention also provides an agent that modulates a physiological characteristic which is associated with gene disruption. In one aspect, the agent is an agonist or antagonist of the phenotype associated with a disruption of a gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide. In yet another aspect, the agent is an agonist or antagonist of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide.

In yet another aspect, the agonist agent is an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody. In still another aspect, the antagonist agent is an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody.

The invention also provides a method of identifying an agent which modulates a behavior associated with a disruption of the gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising:

(a) providing a non-human transgenic animal whose genome comprises a disruption of the gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide;

(b) observing the behavior exhibited by the non-human transgenic animal of (a);

(c) comparing the observed behavior of (b) with that of a gender matched wild-type animal, wherein the observed behavior exhibited by the non-human transgenic animal that differs from the observed behavior exhibited by the wild-type animal is identified as a behavior associated with gene disruption;

(d) administering a test agent to the non-human transgenic animal of (a); and

(e) determining whether the agent modulates the behavior associated with gene disruption.

In one aspect, the observed behavior is an increased anxiety-like response during open field activity testing. In yet another aspect, the observed behavior is a decreased anxiety-like response during open field activity testing. In yet another aspect, the observed behavior is an abnormal circadian rhythm during home-cage activity testing. In yet another aspect, the observed behavior is an enhanced motor coordination during inverted screen testing. In yet another aspect, the observed behavior is impaired motor coordination during inverted screen testing. In yet another aspect, the observed behavior includes depression, generalized anxiety disorders, attention deficit disorder, sleep disorder, hyperactivity disorder, obsessive compulsive disorder, schizophrenia, cognitive disorders, hyperalgesia and sensory disorders. Such disorders include the category defined as “anxiety disorders” which include but are not limited to: mild to moderate anxiety, anxiety disorder due to a general medical condition, anxiety disorder not otherwise specified, generalized anxiety disorder, panic attack, panic disorder with agoraphobia, panic disorder without agoraphobia, posttraumatic stress disorder, social phobia, social anxiety, autism, specific phobia, substance-induced anxiety disorder, acute alcohol withdrawal, obsessive compulsive disorder, agoraphobia, monopolar disorders, bipolar disorder I or II, bipolar disorder not otherwise specified, cyclothymic disorder, depressive disorder, major depressive disorder, mood disorder, substance-induced mood disorder, enhancement of cognitive function, loss of cognitive function associated with but not limited to Alzheimer's disease, stroke, or traumatic injury to the brain, seizures resulting from disease or injury including but not limited to epilepsy, learning disorders/disabilities, cerebral palsy. In addition, anxiety disorders may apply to personality disorders including but not limited to the following types: paranoid, antisocial, avoidant behavior, borderline personality disorders, dependent, histronic, narcissistic, obsessive-compulsive, schizoid, and schizotypal.

The invention also provides an agent that modulates a behavior which is associated with gene disruption. In one aspect, the agent is an agonist or antagonist of the phenotype associated with a disruption of a gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide. In yet another aspect, the agent is an agonist or antagonist of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide. In yet another aspect, the agonist agent is an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody. In still another aspect, the antagonist agent is an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody.

The invention also provides a method of identifying an agent that ameliorates or modulates a neurological disorder; a cardiovascular, endothelial or angiogenic disorder; an eye abnormality; an immunological disorder; an oncological disorder; a bone metabolic abnormality or disorder; a lipid metabolic disorder; or a developmental abnormality associated with a disruption in the gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising:

(a) providing a non-human transgenic animal whose genome comprises a disruption of the gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide;

(b) administering a test agent to said non-human transgenic animal; and

(c) determining whether the test agent ameliorates or modulates the neurological disorder; cardiovascular, endothelial or angiogenic disorder; eye abnormality; immunological disorder; oncological disorder; bone metabolic abnormality or disorder; lipid metabolic disorder; or developmental abnormality associated with the gene disruption in the non-human transgenic animal.

In yet another aspect, the neurological disorder is an increased anxiety-like response during open field activity testing. In yet another aspect, the neurological disorder is a decreased anxiety-like response during open field activity testing. In yet another aspect, the neurological disorder is an abnormal circadian rhythm during home-cage activity testing. In yet another aspect, the neurological disorder is an enhanced motor coordination during inverted screen testing. In yet another aspect, the neurological disorder is impaired motor coordination during inverted screen testing. In yet another aspect, the neurological disorder includes depression, generalized anxiety disorders, attention deficit disorder, sleep disorder, hyperactivity disorder, obsessive compulsive disorder, schizophrenia, cognitive disorders, hyperalgesia and sensory disorders. Such neurological disorders include the category defined as “anxiety disorders” which include but are not limited to: mild to moderate anxiety, anxiety disorder due to a general medical condition, anxiety disorder not otherwise specified, generalized anxiety disorder, panic attack, panic disorder with agoraphobia, panic disorder without agoraphobia, posttraumatic stress disorder, social phobia, social anxiety, autism, specific phobia, substance-induced anxiety disorder, acute alcohol withdrawal, obsessive compulsive disorder, agoraphobia, monopolar disorders, bipolar disorder I or II, bipolar disorder not otherwise specified, cyclothymic disorder, depressive disorder, major depressive disorder, mood disorder, substance-induced mood disorder, enhancement of cognitive function, loss of cognitive function associated with but not limited to Alzheimer's disease, stroke, or traumatic injury to the brain, seizures resulting from disease or injury including but not limited to epilepsy, learning disorders/disabilities, cerebral palsy. In addition, anxiety disorders may apply to personality disorders including but not limited to the following types: paranoid, antisocial, avoidant behavior, borderline personality disorders, dependent, histronic, narcissistic, obsessive-compulsive, schizoid, and schizotypal.

In another aspect, the eye abnormality is a retinal abnormality. In still another aspect, the eye abnormality is consistent with vision problems or blindness. In yet another aspect, the retinal abnormality is consistent with retinitis pigmentosa or is characterized by retinal degeneration or retinal dysplasia.

In still another aspect, the retinal abnormalities the retinal abnormalities are consistent with retinal dysplasia, various retinopathies, including retinopathy of prematurity, retrolental fibroplasia, neovascular glaucoma, age-related macular degeneration, diabetic macular edema, corneal neovascularization, corneal graft neovascularization, corneal graft rejection, retinal/choroidal neovascularization, neovascularization of the angle (rubeosis), ocular neovascular disease, vascular restenosis, arteriovenous malformations (AVM), meningioma, hemangioma, angiofibroma, thyroid hyperplasias (including Grave's disease), corneal and other tissue transplantation, retinal artery obstruction or occlusion; retinal degeneration causing secondary atrophy of the retinal vasculature, retinitis pigmentosa, macular dystrophies, Stargardt's disease, congenital stationary night blindness, choroideremia, gyrate atrophy, Leber's congenital amaurosis, retinoschisis disorders, Wagner's syndrome, Usher syndromes, Zellweger syndrome, Saldino-Mainzer syndrome, Senior-Loken syndrome, Bardet-Biedl syndrome, Alport's syndrome, Alstrom's syndrome, Cockayne's syndrome, dysplaisa spondyloepiphysaria congentia, Flynn-Aird syndrome, Friedreich ataxia, Hallgren syndrome, Marshall syndrome, Albers-Schnoberg disease, Refsum's disease, Kearns-Sayre syndrome, Waardenburg's syndrome, Alagile syndrome, myotonic dystrophy, olivopontocerebellar atrophy, Pierre-Marie dunsdrome, Stickler syndrome, carotinemeia, cystinosis, Wolfram syndrome, Bassen-Kornzweig syndrome, abetalipoproteinemia, incontinentia pigmenti, Batten's disease, mucopolysaccharidoses, homocystinuria, or mannosidosis.

In still another aspect, the eye abnormality is a cataract. In still yet another aspect, the cataract is a systemic disease such as human Down's syndrome, Hallerman-Streiff syndrome, Lowe syndrome, galactosemia, Marfan syndrome, Trismoy 13-15, Alport syndrome, myotonic dystrophy, Fabry disease, hypoparathroidism, or Conradi syndrome.

In still another aspect, the developmental abnormality comprises embryonic lethality or reduced viability.

In yet another aspect, the cardiovascular, endothelial or angiogenic disorders are arterial diseases, such as diabetes mellitus; papilledema; optic atrophy; atherosclerosis; angina; myocardial infarctions such as acute myocardial infarctions, cardiac hypertrophy, and heart failure such as congestive heart failure; hypertension; inflammatory vasculitides; Reynaud's disease and Reynaud's phenomenon; aneurysms and arterial restenosis; venous and lymphatic disorders such as thrombophlebitis, lymphangitis, and lymphedema; peripheral vascular disease; cancer such as vascular tumors, e.g., hemangioma (capillary and cavernous), glomus tumors, telangiectasia, bacillary angiomatosis, hemangioendothelioma, angiosarcoma, haemangiopericytoma, Kaposi's sarcoma, lymphangioma, and lymphangiosarcoma; tumor angiogenesis; trauma such as wounds, burns, and other injured tissue, implant fixation, scarring; ischemia reperfusion injury; rheumatoid arthritis; cerebrovascular disease; renal diseases such as acute renal failure, or osteoporosis.

In still yet another aspect, the immunological disorders are consistent with systemic lupus erythematosis; rheumatoid arthritis; juvenile chronic arthritis; spondyloarthropathies; systemic sclerosis (scleroderma); idiopathic inflammatory myopathies (dermatomyositis, polymyositis); Sjögren's syndrome; systemic vasculitis; sarcoidosis; autoimmune hemolytic anemia (immune pancytopenia, paroxysmal nocturnal hemoglobinuria); autoimmune thrombocytopenia (idiopathic thrombocytopenic purpura, immune-mediated thrombocytopenia); thyroiditis (Grave's disease, Hashimoto's thyroiditis, juvenile lymphocytic thyroiditis, atrophic thyroiditis); diabetes mellitus; immune-mediated renal disease (glomerulonephritis, tubulointerstitial nephritis); demyelinating diseases of the central and peripheral nervous systems such as multiple sclerosis, idiopathic demyelinating polyneuropathy or Guillain-Barré syndrome, and chronic inflammatory demyelinating polyneuropathy; hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis; inflammatory bowel disease (ulcerative colitis: Crohn's disease); gluten-sensitive enteropathy, and Whipple's disease; autoimmune or immune-mediated skin diseases including bullous skin diseases, erythema multiforme and contact dermatitis, psoriasis; allergic diseases such as asthma, allergic rhinitis, atopic dermatitis, food hypersensitivity and urticaria; immunologic diseases of the lung such as eosinophilic pneumonia, idiopathic pulmonary fibrosis and hypersensitivity pneumonitis; or transplantation associated diseases including graft rejection and graft-versus-host disease.

In yet another aspect, the bone metabolic abnormality or disorder is arthritis, osteoporosis, osteopenia or osteopetrosis.

In another aspect, the non-human transgenic animal exhibits at least one of the following physiological characteristics compared with gender matched wild-type littermates: increased anxiety-like response during open field testing; decreased anxiety-like response during open field activity testing; hyperactivity with increased rearing and hole poke activity during open field testing; hypoactivity with decreased rearing and hole poke activity during open field testing; increased exploratory activity during open-field testing; decreased exploratory activity during open-field testing; augmentation of circadian rhythm; abnormal circadian rhythm during home-cage activity testing including decreased ambulatory counts; abnormal circadian rhythm during home-cage activity testing including increased ambulatory counts; enhanced circadian rhythm; increased stress induced hyperthermia with increased stress response; increased resistance to stress induced hyperthermia; decreased resistance to stress induced hyperthermia; impaired motor coordination during inverted screen testing; increased depressive-like response during tail suspension testing; decreased depressive-like response during tail suspension testing; decreased startle response during prepulse inhibition testing; no startle response indicating deafness; reduced latency to respond in hot plate testing; increased pain perception in hot plate testing; prolonged latency to respond in hot plate testing; decreased pain perception in hot plate testing; straub tails during functional observational battery testing; opthamological abnormalities; attenuated retinal arteries; optic nerve abnormalities; retinal degeneration; retinal depigmentation; cataracts; decreased heart rate; decreased mean systolic blood pressure; increased mean systolic blood pressure; increased insulin sensitivity; increased mean fasting serum glucose levels; decreased mean serum glucose levels; increased mean serum cholesterol levels; decreased mean serum cholesterol levels; increased mean serum triglyceride levels; decreased mean serum triglyceride levels; enhanced glucose tolerance; impaired glucose tolerance; decreased mean serum insulin levels; increased uric acid levels; ketonemia; increased mean serum phosphorous levels; increased mean serum potassium levels; increased mean serum alkaline phosphatase levels; decreased mean serum alkaline phosphatase levels; blood in the urine; increased nitrituria; ketonuria; decreased mean serum albumin; decreased mean percentage of natural killer cells; abnormal leukocyte count; increased mean percentage of CD4 cells; decreased mean percentage of CD4 cells; increased mean percentage of B cells in peripheral blood; increase in CD4+ and CD8+ cells with decrease in B cells; decreased B cells and less CD11 blow cells in peritoneum; increased mean percentage B cells in spleen, lymph nodes and Peyer's patches; increase in activated/memory T cells by CD25+ staining and CD62L/CD44 staining; increase in activated/memory T cells in spleen; decreased mean percentage of CD8+ cells; increase total white blood cells (increase in neutrophils, lymphocytes, monocytes and basophils); decreased lymphocytes; increased mean absolute monocyte count; increased mean absolute neutrophil count; decreased mean absolute monocyte count; decreased mean serum IgM, IgA, IgG3, IgG2b and IgG2a levels; decreased mean serum IgG3 levels; decreased mean serum IgM levels; decreased mean serum IgG2a levels; decreased mean serum IgG3 and IgM levels; increase in mean serum IgM levels; increase in mean serum IgG2a levels; increase in mean serum IgG2b levels; anemia; decreased red blood cell count, decreased hemoglobin and decreased hematocrit; increased mean corpuscular volume; increased mean corpuscular hemoglobin; decreased mean corpuscular volume; decreased mean corpuscular hemoglobin; increased red blood cell distribution width and mean platelet volume; decreased red blood cell distribution width; skewed ratios of B220med/CD23− and B220+/CD11− low/CD23− cells after peritoneal lavage; increased CD25 T cells in lymph node and spleen; increased CD38 non-lymphoid cells in Peyer's patches; increased CD23 B cells (peritoneal); decreased percentage of CD4/CD8 DP cells and increased percentage of TCRB+ cells in thymus; decrease in Peyer's patch B cells; reduced number of TCRB+ CD38+ activated T cells in Peyer's patches; increased splenic CD25+ cells and peritoneal CD23 B cells; increased mean platelet count; decreased mean platelet count; decreased mean serum IgG1 response to an ovalbumin challenge; decreased mean serum IgG2a response to an ovalbumin challenge; increased mean serum IgG2a response to an ovalbumin challenge; increased mean serum MCP-1 response to a LPS challenge; increased mean serum TNF-alpha response to a LPS challenge; increased mean serum IL-6 response to a LPS challenge; increased skin fibroblast proliferation; decreased skin fibroblast proliferation; increased mean percent of total body fat and total fat mass; increased mean body weight; increased mean body length; increased total tissue mass (TTM); increased lean body mass (LBM); increased femoral bone mineral density (BMD); increased vertebral bone mineral density (BMD); increased BMC/LBM ratio; increased bone mineral density (BMD); increased total body volumetric bone mineral density (vBMD); increased bone mineral content (BMC); increased mean femoral midshaft cortical thickness and cross-sectional area; increased mean vertebral trabecular bone volume, number and connectivity density; decreased mean percent of total body fat and total fat mass; decreased mean body weight; decreased mean body length; decreased total tissue mass (TTM); decreased lean body mass (LBM); decreased femoral bone mineral density (BMD); decreased vertebral bone mineral density (BMD); decreased BMC/LBM ratio; decreased bone mineral density (BMD); decreased bone mineral content (BMC); decreased volumetric bone mineral density (vBMD); decreased mean femoral midshaft cortical thickness and cross-sectional area; decreased mean vertebral trabecular bone volume, number and connectivity density; myeloid hyperplasia in bone marrow; osteopetrosis with increased bone mineralization; increase in abdominal fat depots; chronic-active arthritis; proliferative chondrapathy and arthropathy; proliferation of cartilage in femoral tibia joints; chondrous metaplasia of cruciate ligaments and perichondral connective tissues; chronic active dermatitis; chronic active inflammation in periarticular tissues; chronic inflammation in various tissues; myeloid hyperplasia in femur and sternum with associated erythroid hyperplasia in the spleen; increased spleen weight; impaired gastrointestinal motility; thymic atrophy; thymic T cell lymphoma; growth retardation; development abnormalities; stunted growth with general reduction in all organ size; growth retardation with reduced viability; and embryonic lethality.

The invention also provides an agent that ameliorates or modulates a neurological disorder; a cardiovascular, endothelial or angiogenic disorder; an eye abnormality; an immunological disorder; an oncological disorder; a bone metabolic abnormality or disorder; a lipid metabolic disorder; or a developmental abnormality which is associated with gene disruption. In one aspect, the agent is an agonist or antagonist of the phenotype associated with a disruption of a gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide. In yet another aspect, the agent is an agonist or antagonist of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide. In yet another aspect, the agonist agent is an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody. In still another aspect, the antagonist agent is an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody.

The invention also provides a therapeutic agent for the treatment of a neurological disorder; a cardiovascular, endothelial or angiogenic disorder; an eye abnormality; an immunologic al disorder; an oncological disorder; a bone metabolic abnormality or disorder; a lipid metabolic disorder; or a developmental abnormality.

The invention also provides a method of identifying an agent that modulates the expression of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising:

(a) contacting a test agent with a host cell expressing a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide; and

(b) determining whether the test agent modulates the expression of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide by the host cell.

The invention also provides an agent that modulates the expression of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide. In one aspect, the agent is an agonist or antagonist of the phenotype associated with a disruption of a gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide. In yet another aspect, the agent is an agonist or antagonist of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide. In yet another aspect, the agonist agent is an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody. In still another aspect, the antagonist agent is an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody.

The invention also provides a method of evaluating a therapeutic agent capable of affecting a condition associated with a disruption of a gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising:

(a) providing a non-human transgenic animal whose genome comprises a disruption of the gene which encodes for the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide;

(b) measuring a physiological characteristic of the non-human transgenic animal of (a);

(c) comparing the measured physiological characteristic of (b) with that of a gender matched wild-type animal, wherein the physiological characteristic of the non-human transgenic animal that differs from the physiological characteristic of the wild-type animal is identified as a condition resulting from the gene disruption in the non-human transgenic animal;

(d) administering a test agent to the non-human transgenic animal of (a); and

(e) evaluating the effects of the test agent on the identified condition associated with gene disruption in the non-human transgenic animal.

In one aspect, the condition is a neurological disorder; a cardiovascular, endothelial or angiogenic disorder; an eye abnormality; an immunological disorder; an oncological disorder; a bone metabolic abnormality or disorder; a lipid metabolic disorder; or a developmental abnormality.

The invention also provides a therapeutic agent which is capable of affecting a condition associated with gene disruption. In one aspect, the agent is an agonist or antagonist of the phenotype associated with a disruption of a gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide. In yet another aspect, the agent is an agonist or antagonist of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide. In yet another aspect, the agonist agent is an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody. In still another aspect, the antagonist agent is an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody.

The invention also provides a pharmaceutical composition comprising a therapeutic agent capable of affecting the condition associated with gene disruption.

The invention also provides a method of treating or preventing or ameliorating a neurological disorder; cardiovascular, endothelial or angiogenic disorder; immunological disorder; oncological disorder; bone metabolic abnormality or disorder, or embryonic lethality associated with the disruption of a gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising administering to a subject in need of such treatment whom may already have the disorder, or may be prone to have the disorder or may be in whom the disorder is to be prevented, a therapeutically effective amount of a therapeutic agent, or agonists or antagonists thereof, thereby effectively treating or preventing or ameliorating said disorder or disease.

In yet another aspect, the neurological disorder is an increased anxiety-like response during open field activity testing. In yet another aspect, the neurological disorder is a decreased anxiety-like response during open field activity testing. In yet another aspect, the neurological disorder is an abnormal circadian rhythm during home-cage activity testing. In yet another aspect, the neurological disorder is an enhanced motor coordination during inverted screen testing. In yet another aspect, the neurological disorder is impaired motor coordination during inverted screen testing. In yet another aspect, the neurological disorder includes depression, generalized anxiety disorders, attention deficit disorder, sleep disorder, hyperactivity disorder, obsessive compulsive disorder, schizophrenia, cognitive disorders, hyperalgesia and sensory disorders. Such neurological disorders include the category defined as “anxiety disorders” which include but are not limited to: mild to moderate anxiety, anxiety disorder due to a general medical condition, anxiety disorder not otherwise specified, generalized anxiety disorder, panic attack, panic disorder with agoraphobia, panic disorder without agoraphobia, posttraumatic stress disorder, social phobia, social anxiety, autism, specific phobia, substance-induced anxiety disorder, acute alcohol withdrawal, obsessive compulsive disorder, agoraphobia, monopolar disorders, bipolar disorder I or II, bipolar disorder not otherwise specified, cyclothymic disorder, depressive disorder, major depressive disorder, mood disorder, substance-induced mood disorder, enhancement of cognitive function, loss of cognitive function associated with but not limited to Alzheimer's disease, stroke, or traumatic injury to the brain, seizures resulting from disease or injury including but not limited to epilepsy, learning disorders/disabilities, cerebral palsy. In addition, anxiety disorders may apply to personality disorders including but not limited to the following types: paranoid, antisocial, avoidant behavior, borderline personality disorders, dependent, histronic, narcissistic, obsessive-compulsive, schizoid, and schizotypal.

In another aspect, the eye abnormality is a retinal abnormality. In still another aspect, the eye abnormality is consistent with vision problems or blindness. In yet another aspect, the retinal abnormality is consistent with retinitis pigmentosa or is characterized by retinal degeneration or retinal dysplasia.

In still another aspect, the retinal abnormalities are consistent with retinal dysplasia, various retinopathies, including retinopathy of prematurity, retrolental fibroplasia, neovascular glaucoma, age-related macular degeneration, diabetic macular edema, corneal neovascularization, corneal graft neovascularization, corneal graft rejection, retinal/choroidal neovascularization, neovascularization of the angle (rubeosis), ocular neovascular disease, vascular restenosis, arteriovenous malformations (AVM), meningioma, hemangioma, angiofibroma, thyroid hyperplasias (including Grave's disease), corneal and other tissue transplantation, retinal artery obstruction or occlusion; retinal degeneration causing secondary atrophy of the retinal vasculature, retinitis pigmentosa, macular dystrophies, Stargardt's disease, congenital stationary night blindness, choroideremia, gyrate atrophy, Leber's congenital amaurosis, retinoschisis disorders, Wagner's syndrome, Usher syndromes, Zellweger syndrome, Saldino-Mainzer syndrome, Senior-Loken syndrome, Bardet-Biedl syndrome, Alport's syndrome, Alstrom's syndrome, Cockayne's syndrome, dysplaisa spondyloepiphysaria congentia, Flynn-Aird syndrome, Friedreich ataxia, Hallgren syndrome, Marshall syndrome, Albers-Schnoberg disease, Refsum's disease, Kearns-Sayre syndrome, Waardenburg's syndrome, Alagile syndrome, myotonic dystrophy, olivopontocerebellar atrophy, Pierre-Marie dunsdrome, Stickler syndrome, carotinemeia, cystinosis, Wolfram syndrome, Bassen-Kornzweig syndrome, abetalipoproteinemia, incontinentia pigmenti, Batten's disease, mucopolysaccharidoses, homocystinuria, or mannosidosis.

In still another aspect, the eye abnormality is a cataract. In still yet another aspect, the cataract is a systemic disease such as human Down's syndrome, Hallerman-Streiff syndrome, Lowe syndrome, galactosemia, Marfan syndrome, Trismoy 13-15, Alport syndrome, myotonic dystrophy, Fabry disease, hypoparathroidism or Conradi syndrome.

In still another aspect, the developmental abnormality comprises embryonic lethality or reduced viability.

In yet another aspect, the cardiovascular, endothelial or angiogenic disorders are arterial diseases, such as diabetes mellitus; papilledema; optic atrophy; atherosclerosis; angina; myocardial infarctions such as acute myocardial infarctions, cardiac hypertrophy, and heart failure such as congestive heart failure; hypertension; inflammatory vasculitides; Reynaud's disease and Reynaud's phenomenon; aneurysms and arterial restenosis; venous and lymphatic disorders such as thrombophlebitis, lymphangitis, and lymphedema; peripheral vascular disease; cancer such as vascular tumors, e.g., hemangioma (capillary and cavernous), glomus tumors, telangiectasia, bacillary angiomatosis, hemangioendothelioma, angiosarcoma, haemangiopericytoma, Kaposi's sarcoma, lymphangioma, and lymphangiosarcoma; tumor angiogenesis; trauma such as wounds, burns, and other injured tissue, implant fixation, scarring; ischemia reperfusion injury; rheumatoid arthritis; cerebrovascular disease; renal diseases such as acute renal failure, or osteoporosis.

In still yet another aspect, the immunological disorders are consistent with systemic lupus erythematosis; rheumatoid arthritis; juvenile chronic arthritis; spondyloarthropathies; systemic sclerosis (scleroderma); idiopathic inflammatory myopathies (dermatomyositis, polymyositis); Sjögren's syndrome; systemic vasculitis; sarcoidosis; autoimmune hemolytic anemia (immune pancytopenia, paroxysmal nocturnal hemoglobinuria); autoimmune thrombocytopenia (idiopathic thrombocytopenic purpura, immune-mediated thrombocytopenia); thyroiditis (Grave's disease, Hashimoto's thyroiditis, juvenile lymphocytic thyroiditis, atrophic thyroiditis); diabetes mellitus; immune-mediated renal disease (glomerulonephritis, tubulointerstitial nephritis); demyelinating diseases of the central and peripheral nervous systems such as multiple sclerosis, idiopathic demyelinating polyneuropathy or Guillain-Barré syndrome, and chronic inflammatory demyelinating polyneuropathy; hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis; inflammatory bowel disease (ulcerative colitis: Crohn's disease); gluten-sensitive enteropathy, and Whipple's disease; autoimmune or immune-mediated skin diseases including bullous skin diseases, erythema multiforme and contact dermatitis, psoriasis; allergic diseases such as asthma, allergic rhinitis, atopic dermatitis, food hypersensitivity and urticaria; immunologic diseases of the lung such as eosinophilic pneumonia, idiopathic pulmonary fibrosis and hypersensitivity pneumonitis; or transplantation associated diseases including graft rejection and graft-versus-host disease.

In yet another aspect, the bone metabolic abnormality or disorder is arthritis, osteoporosis, osteopenia or osteopetrosis.

In another aspect the therapeutic agent is an agonist or antagonist of the phenotype associated with a disruption of a gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide. In yet another aspect, the agent is an agonist or antagonist of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide.

In yet another aspect, the agonist agent is an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody. In still another aspect, the antagonist agent is an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody.

The invention also provides a method of identifying an agent that ameliorates or modulates a neurological disorder; a cardiovascular, endothelial or angiogenic disorder; an eye abnormality; an immunological disorder; an oncological disorder; a bone metabolic abnormality or disorder; a lipid metabolic disorder; or a developmental abnormality associated with a disruption in the gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising:

(a) providing a non-human transgenic animal cell culture, each cell of said culture comprising a disruption of the gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide;

(b) administering a test agent to said cell culture; and

(c) determining whether the test agent ameliorates or modulates the neurological disorder; cardiovascular, endothelial or angiogenic disorder; eye abnormality; immunological disorder; oncological disorder; bone metabolic abnormality or disorder; lipid metabolic disorder; or developmental abnormality in said culture.

In yet another aspect, the neurological disorder is an increased anxiety-like response during open field activity testing. In yet another aspect, the neurological disorder is a decreased anxiety-like response during open field activity testing. In yet another aspect, the neurological disorder is an abnormal circadian rhythm during home-cage activity testing. In yet another aspect, the neurological disorder is an enhanced motor coordination during inverted screen testing. In yet another aspect, the neurological disorder is impaired motor coordination during inverted screen testing. In yet another aspect, the neurological disorder includes depression, generalized anxiety disorders, attention deficit disorder, sleep disorder, hyperactivity disorder, obsessive compulsive disorder, schizophrenia, cognitive disorders, hyperalgesia and sensory disorders. Such neurological disorders include the category defined as “anxiety disorders” which include but are not limited to: mild to moderate anxiety, anxiety disorder due to a general medical condition, anxiety disorder not otherwise specified, generalized anxiety disorder, panic attack, panic disorder with agoraphobia, panic disorder without agoraphobia, posttraumatic stress disorder, social phobia, social anxiety, autism, specific phobia, substance-induced anxiety disorder, acute alcohol withdrawal, obsessive compulsive disorder, agoraphobia, monopolar disorders, bipolar disorder I or II, bipolar disorder not otherwise specified, cyclothymic disorder, depressive disorder, major depressive disorder, mood disorder, substance-induced mood disorder, enhancement of cognitive function, loss of cognitive function associated with but not limited to Alzheimer's disease, stroke, or traumatic injury to the brain, seizures resulting from disease or injury including but not limited to epilepsy, learning disorders/disabilities, cerebral palsy. In addition, anxiety disorders may apply to personality disorders including but not limited to the following types: paranoid, antisocial, avoidant behavior, borderline personality disorders, dependent, histronic, narcissistic, obsessive-compulsive, schizoid, and schizotypal.

In another aspect, the eye abnormality is a retinal abnormality. In still another aspect, the eye abnormality is consistent with vision problems or blindness. In yet another aspect, the retinal abnormality is consistent with retinitis pigmentosa or is characterized by retinal degeneration or retinal dysplasia.

In still another aspect, the retinal abnormalities are consistent with retinal dysplasia, various retinopathies, including retinopathy of prematurity, retrolental fibroplasia, neovascular glaucoma, age-related macular degeneration, diabetic macular edema, corneal neovascularization, corneal graft neovascularization, corneal graft rejection, retinal/choroidal neovascularization, neovascularization of the angle (rubeosis), ocular neovascular disease, vascular restenosis, arteriovenous malformations (AVM), meningioma, hemangioma, angiofibroma, thyroid hyperplasias (including Grave's disease), corneal and other tissue transplantation, retinal artery obstruction or occlusion; retinal degeneration causing secondary atrophy of the retinal vasculature, retinitis pigmentosa, macular dystrophies, Stargardt's disease, congenital stationary night blindness, choroideremia, gyrate atrophy, Leber's congenital amaurosis, retinoschisis disorders, Wagner's syndrome, Usher syndromes, Zellweger syndrome, Saldino-Mainzer syndrome, Senior-Loken syndrome, Bardet-Biedl syndrome, Alport's syndrome, Alstrom's syndrome, Cockayne's syndrome, dysplaisa spondyloepiphysaria congentia, Flynn-Aird syndrome, Friedreich ataxia, Hallgren syndrome, Marshall syndrome, Albers-Schnoberg disease, Refsum's disease, Kearns-Sayre syndrome, Waardenburg's syndrome, Alagile syndrome, myotonic dystrophy, olivopontocerebellar atrophy, Pierre-Marie dunsdrome, Stickler syndrome, carotinemeia, cystinosis, Wolfram syndrome, Bassen-Kornzweig syndrome, abetalipoproteinemia, incontinentiapigmenti, Batten's disease, mucopolysaccharidoses, homocystinuria, or mannosidosis.

In still another aspect, the eye abnormality is a cataract. In still yet another aspect, the cataract is a systemic disease such as human Down's syndrome, Hallerman-Streiff syndrome, Lowe syndrome, galactosemia, Marfan syndrome, Trismoy 13-15, Alport syndrome, myotonic dystrophy, Fabry disease, hypoparathroidism or Conradi syndrome.

In still another aspect, the developmental abnormality comprises embryonic lethality or reduced viability.

In yet another aspect, the cardiovascular, endothelial or angiogenic disorders are arterial diseases, such as diabetes mellitus; papilledema; optic atrophy; atherosclerosis; angina; myocardial infarctions such as acute myocardial infarctions, cardiac hypertrophy, and heart failure such as congestive heart failure; hypertension; inflammatory vasculitides; Reynaud's disease and Reynaud's phenomenon; aneurysms and arterial restenosis; venous and lymphatic disorders such as thrombophlebitis, lymphangitis, and lymphedema; peripheral vascular disease; cancer such as vascular tumors, e.g., hemangioma (capillary and cavernous), glomus tumors, telangiectasia, bacillary angiomatosis, hemangioendothelioma, angiosarcoma, haemangiopericytoma, Kaposi's sarcoma, lymphangioma, and lymphangiosarcoma; tumor angiogenesis; trauma such as wounds, burns, and other injured tissue, implant fixation, scarring; ischemia reperfusion injury; rheumatoid arthritis; cerebrovascular disease; renal diseases such as acute renal failure, or osteoporosis.

In still yet another aspect, the immunological disorders are consistent with systemic lupus erythematosis; rheumatoid arthritis; juvenile chronic arthritis; spondyloarthropathies; systemic sclerosis (scleroderma); idiopathic inflammatory myopathies (dermatomyositis, polymyositis); Sjögren's syndrome; systemic vasculitis; sarcoidosis; autoimmune hemolytic anemia (immune pancytopenia, paroxysmal nocturnal hemoglobinuria); autoimmune thrombocytopenia (idiopathic thrombocytopenic purpura, immune-mediated thrombocytopenia); thyroiditis (Grave's disease, Hashimoto's thyroiditis, juvenile lymphocytic thyroiditis, atrophic thyroiditis); diabetes mellitus; immune-mediated renal disease (glomerulonephritis, tubulointerstitial nephritis); demyelinating diseases of the central and peripheral nervous systems such as multiple sclerosis, idiopathic demyelinating polyneuropathy or Guillain-Barré syndrome, and chronic inflammatory demyelinating polyneuropathy; hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis; inflammatory bowel disease (ulcerative colitis: Crohn's disease); gluten-sensitive enteropathy, and Whipple's disease; autoimmune or immune-mediated skin diseases including bullous skin diseases, erythema multiforme and contact dermatitis, psoriasis; allergic diseases such as asthma, allergic rhinitis, atopic dermatitis, food hypersensitivity and urticaria; immunologic diseases of the lung such as eosinophilic pneumonia, idiopathic pulmonary fibrosis and hypersensitivity pneumonitis; or transplantation associated diseases including graft rejection and graft-versus-host disease.

In yet another aspect, the bone metabolic abnormality or disorder is arthritis, osteoporosis, osteopenia or osteopetrosis.

The invention also provides an agent that ameliorates or modulates a neurological disorder; a cardiovascular, endothelial or angiogenic disorder; an eye abnormality; an immunological disorder; an oncological disorder; a bone metabolic abnormality or disorder; a lipid metabolic disorder; or a developmental abnormality which is associated with gene disruption in said culture. In one aspect, the agent is an agonist or antagonist of the phenotype associated with a disruption of a gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide. In yet another aspect, the agent is an agonist or antagonist of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide. In yet another aspect, the agonist agent is an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody. In still another aspect, the antagonist agent is an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody.

The invention also provides a method of modulating a phenotype associated with a disruption of a gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising administering to a subject whom may already have the phenotype, or may be prone to have the phenotype or may be in whom the phenotype is to be prevented, an effective amount of an agent identified as modulating said phenotype, or agonists or antagonists thereof, thereby effectively modulating the phenotype.

The invention also provides a method of modulating a physiological characteristic associated with a disruption of a gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising administering to a subject whom may already exhibit the physiological characteristic, or may be prone to exhibit the physiological characteristic or may be in whom the physiological characteristic is to be prevented, an effective amount of an agent identified as modulating said physiologic al characteristic, or agonists or antagonists thereof, thereby effectively modulating the physiological characteristic.

The invention also provides a method of modulating a behavior associated with a disruption of a gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising administering to a subject whom may already exhibit the behavior, or may be prone to exhibit the behavior or may be in whom the exhibited behavior is to be prevented, an effective amount of an agent identified as modulating said behavior, or agonists or antagonists thereof, thereby effectively modulating the behavior.

The invention also provides a method of modulating the expression of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising administering to a host cell expressing said PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, an effective amount of an agent identified as modulating said expression, or agonists or antagonists thereof, thereby effectively modulating the expression of said polypeptide.

The invention also provides a method of modulating a condition associated with a disruption of a gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising administering to a subject whom may have the condition, or may be prone to have the condition or may be in whom the condition is to be prevented, a therapeutically effective amount of a therapeutic agent identified as modulating said condition, or agonists or antagonists thereof, thereby effectively modulating the condition.

The invention also provides a method of treating or preventing or ameliorating a neurological disorder; cardiovascular, endothelial or angiogenic disorder; immunological disorder; oncological disorder; bone metabolic abnormality or disorder, or embryonic lethality associated with the disruption of a gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising administering to a non-human transgenic animal cell culture, each cell of said culture comprising a disruption of the gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, an effective amount of an agent identified as treating or preventing or ameliorating said disorder, or agonists or antagonists thereof, thereby effectively treating or preventing or ameliorating said disorder.

### B. Further Embodiments

In yet further embodiments, the invention is directed to the following set of potential claims for this application:

1. A method of identifying a phenotype associated with a disruption of a gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising:

(a) providing a non-human transgenic animal whose genome comprises a disruption of the gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide;

(b) measuring a physiological characteristic of the non-human transgenic animal; and

(c) comparing the measured physiologic al characteristic with that of a gender matched wild-type animal, wherein the physiological characteristic of the non-human transgenic animal that differs from the physiological characteristic of the wild-type animal is identified as a phenotype resulting from the gene disruption in the non-human transgenic animal.

2. The method of claim 1, wherein the non-human transgenic animal is heterozygous for the disruption of a gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide.
3. The method of claim 1, wherein the phenotype exhibited by the non-human transgenic animal as compared with gender matched wild-type littermates is at least one of the following: a neurological disorder; a cardiovascular, endothelial or angiogenic disorder; an eye abnormality; an immunological disorder; an oncological disorder; a bone metabolic abnormality or disorder; a lipid metabolic disorder; or a developmental abnormality.
4. The method of claim 3, wherein the neurological disorder is an increased anxiety-like response during open field activity testing.
5. The method of claim 3, wherein the neurological disorder is a decreased anxiety-like response during open field activity testing.
6. The method of claim 3, wherein the neurological disorder is an abnormal circadian rhythm during home-cage activity testing.
7. The method of claim 3, wherein the neurological disorder is an enhanced motor coordination during inverted screen testing.
8. The method of claim 3, wherein the neurological disorder is an impaired motor coordination during inverted screen testing.
9. The method of claim 3, wherein the neurological disorder is depression, generalized anxiety disorders, attention deficit disorder, sleep disorder, hyperactivity disorder, obsessive compulsive disorder, schizophrenia, cognitive disorders, hyperalgesia or sensory disorders.
10. The method of claim 3, wherein the eye abnormality is a retinal abnormality.
11. The method of claim 3, wherein the eye abnormality is consistent with vision problems or blindness.
12. The method of claim 10, wherein the retinal abnormality is consistent with retinitis pigmentosa.
13. The method of claim 10, wherein the retinal abnormality is characterized by retinal degeneration or retinal dysplasia.
14. The method of claim 10, wherein the retinal abnormality is consistent with retinal dysplasia, various retinopathies, including retinopathy of prematurity, retrolental fibroplasia, neovascular glaucoma, age-related macular degeneration, diabetic macular edema, corneal neovascularization, corneal graft neovascularization, corneal graft rejection, retinal/choroidal neovascularization, neovascularization of the angle (rubeosis), ocular neovascular disease, vascular restenosis, arteriovenous malformations (AVM), meningioma, hemangioma, angiofibroma, thyroid hyperplasias (including Grave's disease), corneal and other tissue transplantation, retinal artery obstruction or occlusion; retinal degeneration causing secondary atrophy of the retinal vasculature, retinitis pigmentosa, macular dystrophies, Stargardt's disease, congenital stationary night blindness, choroideremia, gyrate atrophy, Leber's congenital amaurosis, retinoschisis disorders, Wagner's syndrome, Usher syndromes, Zellweger syndrome, Saldino-Mainzer syndrome, Senior-Loken syndrome, Bardet-Biedl syndrome, Alport's syndrome, Alstrom's syndrome, Cockayne's syndrome, dysplaisa spondyloepiphysaria congentia, Flynn-Aird syndrome, Friedreich ataxia, Hallgren syndrome, Marshall syndrome, Albers-Schnoberg disease, Refsum's disease, Kearns-Sayre syndrome, Waardenburg's syndrome, Alagile syndrome, myotonic dystrophy, olivopontocerebellar atrophy, Pierre-Marie dunsdrome, Stickler syndrome, carotinemeia, cystinosis, Wolfram syndrome, Bassen-Kornzweig syndrome, abetalipoproteinemia, incontinentia pigmenti, Batten's disease, mucopolysaccharidoses, homocystinuria, or mannosidosis.
15. The method of claim 3, wherein the eye abnormality is a cataract.
16. The method of claim 15, wherein the cataract is consistent with systemic diseases such as human Down's syndrome, Hallerman-Streiff syndrome, Lowe syndrome, galactosemia, Marfan syndrome, Trismoy 13-15, Alport syndrome, myotonic dystrophy, Fabry disease, hypoparathroidism or Conradi syndrome.
17. The method of claim 3, wherein the developmental abnormality comprises embryonic lethality or reduced viability.
18. The method of claim 3, wherein the cardiovascular, endothelial or angiogenic disorders are arterial diseases, such as diabetes mellitus; papilledema; optic atrophy; atherosclerosis; angina; myocardial infarctions such as acute myocardial infarctions, cardiac hypertrophy, and heart failure such as congestive heart failure; hypertension; inflammatory vasculitides; Reynaud's disease and Reynaud's phenomenon; aneurysms and arterial restenosis; venous and lymphatic disorders such as thrombophlebitis, lymphangitis, and lymphedema; peripheral vascular disease; cancer such as vascular tumors, e.g., hemangioma (capillary and cavernous), glomus tumors, telangiectasia, bacillary angiomatosis, hemangioendothelioma, angiosarcoma, haemangiopericytoma, Kaposi's sarcoma, lymphangioma, and lymphangiosarcoma; tumor angiogenesis; trauma such as wounds, burns, and other injured tissue, implant fixation, scarring; ischemia reperfusion injury; rheumatoid arthritis; cerebrovascular disease; renal diseases such as acute renal failure, or osteoporosis.
19. The method of claim 3, wherein the immunological disorders are systemic lupus erythematosis; rheumatoid arthritis; juvenile chronic arthritis; spondyloarthropathies; systemic sclerosis (scleroderma); idiopathic inflammatory myopathies (dermatomyositis, polymyositis); Sjögren's syndrome; systemic vasculitis; sarcoidosis; autoimmune hemolytic anemia (immune pancytopenia, paroxysmal nocturnal hemoglobinuria); autoimmune thrombocytopenia (idiopathic thrombocytopenic purpura, immune-mediated thrombocytopenia); thyroiditis

(Grave's disease, Hashimoto's thyroiditis, juvenile lymphocytic thyroiditis, atrophic thyroiditis); diabetes mellitus; immune-mediated renal disease (glomerulonephritis, tubulointerstitial nephritis); demyelinating diseases of the central and peripheral nervous systems such as multiple sclerosis, idiopathic demyelinating polyneuropathy or Guillain-Barré syndrome, and chronic inflammatory demyelinating polyneuropathy; hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis; inflammatory bowel disease (ulcerative colitis: Crohn's disease); gluten-sensitive enteropathy, and Whipple's disease; autoimmune or immune-mediated skin diseases including bullous skin diseases, erythema multiforme and contact dermatitis, psoriasis; allergic diseases such as asthma, allergic rhinitis, atopic dermatitis, food hypersensitivity and urticaria; immunologic diseases of the lung such as eosinophilic pneumonia, idiopathic pulmonary fibrosis and hypersensitivity pneumonitis; or transplantation associated diseases including graft rejection and graft-versus-host disease.

20. The method of claim 3, wherein the bone metabolic abnormality or disorder is arthritis, osteoporosis or osteopetrosis.
21. The method of claim 1, wherein the non-human transgenic animal exhibits at least one of the following physiological characteristics compared with gender matched wild-type littermates: increased anxiety-like response during open field testing; decreased anxiety-like response during open field activity testing; hyperactivity with increased rearing and hole poke activity during open field testing; hypoactivity with decreased rearing and hole poke activity during open field testing; increased exploratory activity during open-field testing; decreased exploratory activity during open-field testing; augmentation of circadian rhythm; abnormal circadian rhythm during home-cage activity testing including decreased ambulatory counts; abnormal circadian rhythm during home-cage activity testing including increased ambulatory counts; enhanced circadian rhythm; increased stress induced hyperthermia with increased stress response; increased resistance to stress induced hyperthermia; decreased resistance to stress induced hyperthermia; impaired motor coordination during inverted screen testing; increased depressive-like response during tail suspension testing; decreased depressive-like response during tail suspension testing; decreased startle response during prepulse inhibition testing; no startle response indicating deafness; reduced latency to respond in hot plate testing; increased pain perception in hot plate testing; prolonged latency to respond in hot plate testing; decreased pain perception in hot plate testing; straub tails during functional observational battery testing; opthamological abnormalities; attenuated retinal arteries; optic nerve abnormalities; retinal degeneration; retinal depigmentation; cataracts; decreased heart rate; decreased mean systolic blood pressure; increased mean systolic blood pressure; increased insulin sensitivity; increased mean fasting serum glucose levels; decreased mean serum glucose levels; increased mean serum cholesterol levels; decreased mean serum cholesterol levels; increased mean serum triglyceride levels; decreased mean serum triglyceride levels; enhanced glucose tolerance; impaired glucose tolerance; decreased mean serum insulin levels; increased uric acid levels; ketonemia; increased mean serum phosphorous levels; increased mean serum potassium levels; increased mean serum alkaline phosphatase levels; decreased mean serum alkaline phosphatase levels; blood in the urine; increased nitrituria; ketonuria; decreased mean serum albumin; decreased mean percentage of natural killer cells; abnormal leukocyte count; increased mean percentage of CD4 cells; decreased mean percentage of CD4 cells; increased mean percentage of B cells in peripheral blood; increase in CD4+ and CD8+ cells with decrease in B cells; decreased B cells and less CD11 blow cells in peritoneum; increased mean percentage B cells in spleen, lymph nodes and Peyer's patches; increase in activated/memory T cells by CD25+ staining and CD62L/CD44 staining; increase in activated/memory T cells in spleen; decreased mean percentage of CD8+ cells; increase total white blood cells (increase in neutrophils, lymphocytes, monocytes and basophils); decreased lymphocytes; increased mean absolute monocyte count; increased mean absolute neutrophil count; decreased mean absolute monocyte count; decreased mean serum IgM, IgA, IgG3, IgG2b and IgG2a levels; decreased mean serum IgG3 levels; decreased mean serum IgM levels; decreased mean serum IgG2a levels; decreased mean serum IgG3 and IgM levels; increase in mean serum IgM levels; increase in mean serum IgG2a levels; increase in mean serum IgG2b levels; anemia; decreased red blood cell count, decreased hemoglobin and decreased hematocrit; increased mean corpuscular volume; increased mean corpuscular hemoglobin; decreased mean corpuscular volume; decreased mean corpuscular hemoglobin; increased red blood cell distribution width and mean platelet volume; decreased red blood cell distribution width; skewed ratios of B220med/CD23− and B220+/CD11− low/CD23− cells after peritoneal lavage; increased CD25 T cells in lymph node and spleen; increased CD38 non-lymphoid cells in Peyer's patches; increased CD23 B cells (peritoneal); decreased percentage of CD4/CD8 DP cells and increased percentage of TCRB+ cells in thymus; decrease in Peyer's patch B cells; reduced number of TCRB+ CD38+ activated T cells in Peyer's patches; increased splenic CD25+ cells and peritoneal CD23 B cells; increased mean platelet count; decreased mean platelet count; decreased mean serum IgG1 response to an ovalbumin challenge; decreased mean serum IgG2a response to an ovalbumin challenge; increased mean serum IgG2a response to an ovalbumin challenge; increased mean serum MCP-1 response to a LPS challenge; increased mean serum TNF-alpha response to a LPS challenge; increased mean serum IL-6 response to a LPS challenge; increased skin fibroblast proliferation; decreased skin fibroblast proliferation; increased mean percent of total body fat and total fat mass; increased mean body weight; increased mean body length; increased total tissue mass (TTM); increased lean body mass (LBM); increased femoral bone mineral density (BMD); increased vertebral bone mineral density (BMD); increased BMC/LBM ratio; increased bone mineral density (BMD); increased total body volumetric bone mineral density (vBMD); increased bone mineral content (BMC); increased mean femoral midshaft cortical thickness and cross-sectional area; increased mean vertebral trabecular bone volume, number and connectivity density; decreased mean percent of total body fat and total fat mass; decreased mean body weight; decreased mean body length; decreased total tissue mass (TTM); decreased lean body mass (LBM); decreased femoral bone mineral density (BMD); decreased vertebral bone mineral density (BMD); decreased BMC/LBM ratio; decreased bone mineral density (BMD); decreased bone mineral content (BMC); decreased volumetric bone mineral density (vBMD); decreased mean femoral midshaft cortical thickness and cross-sectional area; decreased mean vertebral trabecular bone volume, number and connectivity density; myeloid hyperplasia in bone marrow; osteopetrosis with increased bone mineralization; increase in abdominal fat depots; chronic-active arthritis; proliferative chondrapathy and arthropathy; proliferation of cartilage in femoral tibia joints; chondrous metaplasia of cruciate ligaments and perichondral connective tissues; chronic active dermatitis; chronic active inflammation in periarticular tissues; chronic inflammation in various tissues; myeloid hyperplasia in femur and sternum with associated erythroid hyperplasia in the spleen; increased spleen weight; impaired gastrointestinal motility; thymic atrophy; thymic T cell lymphoma; growth retardation; development abnormalities; stunted growth with general reduction in all organ size; growth retardation with reduced viability; and embryonic lethality.
22. An isolated cell derived from a non-human transgenic animal whose genome comprises a disruption of the gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide.
23. The isolated cell of claim 22 which is a murine cell.
24. The isolated cell of claim 23, wherein the murine cell is an embryonic stem cell.
25. The isolated cell of claim 22, wherein the non-human transgenic animal exhibits at least one of the following phenotypes compared with gender matched wild-type littermates: a neurological disorder; a cardiovascular, endothelial or angiogenic disorder; an eye abnormality; an immunological disorder; an oncological disorder; a bone metabolic abnormality or disorder; a lipid metabolic disorder; or a developmental abnormality.
26. A method of identifying an agent that modulates a phenotype associated with a disruption of a gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising:

(a) providing a non-human transgenic animal whose genome comprises a disruption of the gene which encodes for the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide;

(b) measuring a physiological characteristic of the non-human transgenic animal of (a);

(c) comparing the measured physiological characteristic of (b) with that of a gender matched wild-type animal, wherein the physiological characteristic of the non-human transgenic animal that differs from the physiological characteristic of the wild-type animal is identified as a phenotype resulting from the gene disruption in the non-human transgenic animal;

(d) administering a test agent to the non-human transgenic animal of (a); and

(e) determining whether the test agent modulates the identified phenotype associated with gene disruption in the non-human transgenic animal.

27. The method of claim 26, wherein the phenotype associated with the gene disruption comprises a neurological disorder; a cardiovascular, endothelial or angiogenic disorder; an eye abnormality; an immunological disorder; an oncological disorder; a bone metabolic abnormality or disorder; a lipid metabolic disorder; or a developmental abnormality.
28. The method of claim 27, wherein the neurological disorder is an increased anxiety-like response during open field activity testing.
29. The method of claim 27, wherein the neurological disorder is a decreased anxiety-like response during open field activity testing.
30. The method of claim 27, wherein the neurological disorder is an abnormal circadian rhythm during home-cage activity testing.
31. The method of claim 27, wherein the neurological disorder is an enhanced motor coordination during inverted screen testing.
32. The method of claim 27, wherein the neurological disorder is an impaired motor coordination during inverted screen testing.
33. The method of claim 27, wherein the neurological disorder is depression, generalized anxiety disorders, attention deficit disorder, sleep disorder, hyperactivity disorder, obsessive compulsive disorder, schizophrenia, cognitive disorders, hyperalgesia or sensory disorders.
34. The method of claim 27, wherein the eye abnormality is a retinal abnormality.
35. The method of claim 27, wherein the eye abnormality is consistent with vision problems or blindness.
36. The method of claim 34, wherein the retinal abnormality is consistent with retinitis pigmentosa.
37. The method of claim 34, wherein the retinal abnormality is characterized by retinal degeneration or retinal dysplasia.
38. The method of claim 34, wherein the retinal abnormality is consistent with retinal dysplasia, various retinopathies, including retinopathy of prematurity, retrolental fibroplasia, neovascular glaucoma, age-related macular degeneration, diabetic macular edema, corneal neovascularization, corneal graft neovascularization, corneal graft rejection, retinal/choroidal neovascularization, neovascularization of the angle (rubeosis), ocular neovascular disease, vascular restenosis, arteriovenous malformations (AVM), meningioma, hemangioma, angiofibroma, thyroid hyperplasias (including Grave's disease), corneal and other tissue transplantation, retinal artery obstruction or occlusion; retinal degeneration causing secondary atrophy of the retinal vasculature, retinitis pigmentosa, macular dystrophies, Stargardt's disease, congenital stationary night blindness, choroideremia, gyrate atrophy, Leber's congenital amaurosis, retinoschisis disorders, Wagner's syndrome, Usher syndromes, Zellweger syndrome, Saldino-Mainzer syndrome, Senior-Loken syndrome, Bardet-Biedl syndrome, Alport's syndrome, Alstrom's syndrome, Cockayne's syndrome, dysplaisa spondyloepiphysaria congentia, Flynn-Aird syndrome, Friedreich ataxia, Hallgren syndrome, Marshall syndrome, Albers-Schnoberg disease, Refsum's disease, Kearns-Sayre syndrome, Waardenburg's syndrome, Alagile syndrome, myotonic dystrophy, olivopontocerebellar atrophy, Pierre-Marie dunsdrome, Stickler syndrome, carotinemeia, cystinosis, Wolfram syndrome, Bassen-Kornzweig syndrome abetalipoproteinemia, incontinentia pigmenti, Batten's disease, mucopolysaccharidoses, homocystinuria, or mannosidosis.
39. The method of claim 27, wherein the eye abnormality is a cataract.
40. The method of claim 39, wherein the cataract is consistent with systemic diseases such as human Down's syndrome, Hallerman-Streiff syndrome, Lowe syndrome, galactosemia, Marfan syndrome, Trismoy 13-15, Alport syndrome, myotonic dystrophy, Fabry disease, hypoparathroidism or Conradi syndrome.
41. The method of claim 27, wherein the developmental abnormality comprises embryonic lethality or reduced viability.
42. The method of claim 27, wherein the cardiovascular, endothelial or angiogenic disorders are arterial diseases, such as diabetes mellitus; papilledema; optic atrophy; atherosclerosis; angina; myocardial infarctions such as acute myocardial infarctions, cardiac hypertrophy, and heart failure such as congestive heart failure; hypertension; inflammatory vasculitides; Reynaud's disease and Reynaud's phenomenon; aneurysms and arterial restenosis; venous and lymphatic disorders such as thrombophlebitis, lymphangitis, and lymphedema; peripheral vascular disease; cancer such as vascular tumors, e.g., hemangioma (capillary and cavernous), glomus tumors, telangiectasia, bacillary angiomatosis, hemangioendothelioma, angiosarcoma, haemangiopericytoma, Kaposi's sarcoma, lymphangioma, and lymphangiosarcoma; tumor angiogenesis; trauma such as wounds, burns, and other injured tissue, implant fixation, scarring; ischemiareperfusion injury; rheumatoid arthritis; cerebrovascular disease; renal diseases such as acute renal failure, or osteoporosis.
43. The method of claim 27, wherein the immunological disorders are systemic lupus erythematosis; rheumatoid arthritis; juvenile chronic arthritis; spondyloarthropathies; systemic sclerosis (scleroderma); idiopathic inflammatory myopathies (dermatomyositis, polymyositis); Sjögren's syndrome; systemic vasculitis; sarcoidosis; autoimmune hemolytic anemia (immune pancytopenia, paroxysmal nocturnal hemoglobinuria); autoimmune thrombocytopenia (idiopathic thrombocytopenic purpura, immune-mediated thrombocytopenia); thyroiditis (Grave's disease, Hashimoto's thyroiditis, juvenile lymphocytic thyroiditis, atrophic thyroiditis); diabetes mellitus; immune-mediated renal disease (glomerulonephritis, tubulointerstitial nephritis); demyelinating diseases of the central and peripheral nervous systems such as multiple sclerosis, idiopathic demyelinating polyneuropathy or Guillain-Barré syndrome, and chronic inflammatory demyelinating polyneuropathy; hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis; inflammatory bowel disease (ulcerative colitis: Crohn's disease); gluten-sensitive enteropathy, and Whipple's disease; autoimmune or immune-mediated skin diseases including bullous skin diseases, erythema multiforme and contact dermatitis, psoriasis; allergic diseases such as asthma, allergic rhinitis, atopic dermatitis, food hypersensitivity and urticaria; immunologic diseases of the lung such as eosinophilic pneumonia, idiopathic pulmonary fibrosis and hypersensitivity pneumonitis; or transplantation-associated diseases including graft rejection and graft-versus-host disease.
44. The method of claim 27, wherein said bone metabolic abnormality or disorder is arthritis, osteoporosis or osteopetrosis.
45. The method of claim 26, wherein the non-human transgenic animal exhibits at least one of the following physiological characteristics compared with gender matched wild-type littermates: increased anxiety-like response during open field testing; decreased anxiety-like response during open field activity testing; hyperactivity with increased rearing and hole poke activity during open field testing; hypoactivity with decreased rearing and hole poke activity during open field testing; increased exploratory activity during open-field testing; decreased exploratory activity during open-field testing; augmentation of circadian rhythm; abnormal circadian rhythm during home-cage activity testing including decreased ambulatory counts; abnormal circadian rhythm during home-cage activity testing including increased ambulatory counts; enhanced circadian rhythm; increased stress induced hyperthermia with increased stress response; increased resistance to stress induced hyperthermia; decreased resistance to stress induced hyperthermia; impaired motor coordination during inverted screen testing; increased depressive-like response during tail suspension testing; decreased depressive-like response during tail suspension testing; decreased startle response during prepulse inhibition testing; no startle response indicating deafness; reduced latency to respond in hot plate testing; increased pain perception in hot plate testing; prolonged latency to respond in hot plate testing; decreased pain perception in hot plate testing; straub tails during functional observational battery testing; opthamological abnormalities; attenuated retinal arteries; optic nerve abnormalities; retinal degeneration; retinal depigmentation; cataracts; decreased heart rate; decreased mean systolic blood pressure; increased mean systolic blood pressure; increased insulin sensitivity; increased mean fasting serum glucose levels; decreased mean serum glucose levels; increased mean serum cholesterol levels; decreased mean serum cholesterol levels; increased mean serum triglyceride levels; decreased mean serum triglyceride levels; enhanced glucose tolerance; impaired glucose tolerance; decreased mean serum insulin levels; increased uric acid levels; ketonemia; increased mean serum phosphorous levels; increased mean serum potassium levels; increased mean serum alkaline phosphatase levels; decreased mean serum alkaline phosphatase levels; blood in the urine; increased nitrituria; ketonuria; decreased mean serum albumin; decreased mean percentage of natural killer cells; abnormal leukocyte count; increased mean percentage of CD4 cells; decreased mean percentage of CD4 cells; increased mean percentage of B cells in peripheral blood; increase in CD4+ and CD8+ cells with decrease in B cells; decreased B cells and less CD11 blow cells in peritoneum; increased mean percentage B cells in spleen, lymph nodes and Peyer's patches; increase in activated/memory T cells by CD25+ staining and CD62L/CD44 staining; increase in activated/memory T cells in spleen; decreased mean percentage of CD8+ cells; increase total white blood cells (increase in neutrophils, lymphocytes, monocytes and basophils); decreased lymphocytes; increased mean absolute monocyte count; increased mean absolute neutrophil count; decreased mean absolute monocyte count; decreased mean serum IgM, IgA, IgG3, IgG2b and IgG2a levels; decreased mean serum IgG3 levels; decreased mean serum IgM levels; decreased mean serum IgG2a levels; decreased mean serum IgG3 and IgM levels; increase in mean serum IgM levels; increase in mean serum IgG2a levels; increase in mean serum IgG2b levels; anemia; decreased red blood cell count, decreased hemoglobin and decreased hematocrit; increased mean corpuscular volume; increased mean corpuscular hemoglobin; decreased mean corpuscular volume; decreased mean corpuscular hemoglobin; increased red blood cell distribution width and mean platelet volume; decreased red blood cell distribution width; skewed ratios of B220med/CD23− and B220+/CD11− low/CD23− cells after peritoneal lavage; increased CD25 T cells in lymph node and spleen; increased CD38 non-lymphoid cells in Peyer's patches; increased CD23 B cells (peritoneal); decreased percentage of CD4/CD8 DP cells and increased percentage of TCRB+ cells in thymus; decrease in Peyer's patch B cells; reduced number of TCRB+ CD38+ activated T cells in Peyer's patches; increased splenic CD25+ cells and peritoneal CD23 B cells; increased mean platelet count; decreased mean platelet count; decreased mean serum IgG1 response to an ovalbumin challenge; decreased mean serum IgG2a response to an ovalbumin challenge; increased mean serum IgG2a response to an ovalbumin challenge; increased mean serum MCP-1 response to a LPS challenge; increased mean serum TNF-alpha response to a LPS challenge; increased mean serum IL-6 response to a LPS challenge; increased skin fibroblast proliferation; decreased skin fibroblast proliferation; increased mean percent of total body fat and total fat mass; increased mean body weight; increased mean body length; increased total tissue mass (TTM); increased lean body mass (LBM); increased femoral bone mineral density (BMD); increased vertebral bone mineral density (BMD); increased BMC/LBM ratio; increased bone mineral density (BMD); increased total body volumetric bone mineral density (vBMD); increased bone mineral content (BMC); increased mean femoral midshaft cortical thickness and cross-sectional area; increased mean vertebral trabecular bone volume, number and connectivity density; decreased mean percent of total body fat and total fat mass; decreased mean body weight; decreased mean body length; decreased total tissue mass (TTM); decreased lean body mass (LBM); decreased femoral bone mineral density (BMD); decreased vertebral bone mineral density (BMD); decreased BMC/LBM ratio; decreased bone mineral density (BMD); decreased bone mineral content (BMC); decreased volumetric bone mineral density (vBMD); decreased mean femoral midshaft cortical thickness and cross-sectional area; decreased mean vertebral trabecular bone volume, number and connectivity density; myeloid hyperplasia in bone marrow; osteopetrosis with increased bone mineralization; increase in abdominal fat depots; chronic-active arthritis; proliferative chondrapathy and arthropathy; proliferation of cartilage in femoral tibia joints; chondrous metaplasia of cruciate ligaments and perichondral connective tissues; chronic active dermatitis; chronic active inflammation in periarticular tissues; chronic inflammation in various tissues; myeloid hyperplasia in femur and sternum with associated erythroid hyperplasia in the spleen; increased spleen weight; impaired gastrointestinal motility; thymic atrophy; thymic T cell lymphoma; growth retardation; development abnormalities; stunted growth with general reduction in all organ size; growth retardation with reduced viability; and embryonic lethality.
46. An agent identified by the method of claim 26.
47. The agent of claim 46 which is an agonist or antagonist of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide.
48. The agent of claim 47, wherein the agonist is an anti-PRO179, anti-PRO181, anti-PRO244, anti-PRO247,
anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody.
49. The agent of claim 47, wherein the antagonist is an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody.
50. A method of identifying an agent that modulates a physiological characteristic associated with a disruption of the gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising:

(a) providing a non-human transgenic animal whose genome comprises a disruption of the gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide;

(b) measuring a physiological characteristic exhibited by the non-human transgenic animal of (a);

(c) comparing the measured physiological characteristic of (b) with that of a gender matched wild-type animal, wherein the physiological characteristic exhibited by the non-human transgenic animal that differs from the physiological characteristic exhibited by the wild-type animal is identified as a physiological characteristic associated with gene disruption;

(d) administering a test agent to the non-human transgenic animal of (a); and

(e) determining whether the physiological characteristic associated with gene disruption is modulated.

51. The method of claim 50, wherein the non-human transgenic animal exhibits at least one of the following physiological characteristics compared with gender matched wild-type littermates: increased anxiety-like response during open field testing; decreased anxiety-like response during open field activity testing; hyperactivity with increased rearing and hole poke activity during open field testing; hypoactivity with decreased rearing and hole poke activity during open field testing; increased exploratory activity during open-field testing; decreased exploratory activity during open-field testing; augmentation of circadian rhythm; abnormal circadian rhythm during home-cage activity testing including decreased ambulatory counts; abnormal circadian rhythm during home-cage activity testing including increased ambulatory counts; enhanced circadian rhythm; increased stress induced hyperthermia with increased stress response; increased resistance to stress induced hyperthermia; decreased resistance to stress induced hyperthermia; impaired motor coordination during inverted screen testing; increased depressive-like response during tail suspension testing; decreased depressive-like response during tail suspension testing; decreased startle response during prepulse inhibition testing; no startle response indicating deafness; reduced latency to respond in hot plate testing; increased pain perception in hot plate testing; prolonged latency to respond in hot plate testing; decreased pain perception in hot plate testing; straub tails during functional observational battery testing; opthamological abnormalities; attenuated retinal arteries; optic nerve abnormalities; retinal degeneration; retinal depigmentation; cataracts; decreased heart rate; decreased mean systolic blood pressure; increased mean systolic blood pressure; increased insulin sensitivity; increased mean fasting serum glucose levels; decreased mean serum glucose levels; increased mean serum cholesterol levels; decreased mean serum cholesterol levels; increased mean serum triglyceride levels; decreased mean serum triglyceride levels; enhanced glucose tolerance; impaired glucose tolerance; decreased mean serum insulin levels; increased uric acid levels; ketonemia; increased mean serum phosphorous levels; increased mean serum potassium levels; increased mean serum alkaline phosphatase levels; decreased mean serum alkaline phosphatase levels; blood in the urine; increased nitrituria; ketonuria; decreased mean serum albumin; decreased mean percentage of natural killer cells; abnormal leukocyte count; increased mean percentage of CD4 cells; decreased mean percentage of CD4 cells; increased mean percentage of B cells in peripheral blood; increase in CD4+ and CD8+ cells with decrease in B cells; decreased B cells and less CD11 blow cells in peritoneum; increased mean percentage B cells in spleen, lymph nodes and Peyer's patches; increase in activated/memory T cells by CD25+ staining and CD62L/CD44 staining; increase in activated/memory T cells in spleen; decreased mean percentage of CD8+ cells; increase total white blood cells (increase in neutrophils, lymphocytes, monocytes and basophils); decreased lymphocytes; increased mean absolute monocyte count; increased mean absolute neutrophil count; decreased mean absolute monocyte count; decreased mean serum IgM, IgA, IgG3, IgG2b and IgG2a levels; decreased mean serum IgG3 levels; decreased mean serum IgM levels; decreased mean serum IgG2a levels; decreased mean serum IgG3 and IgM levels; increase in mean serum IgM levels; increase in mean serum IgG2a levels; increase in mean serum IgG2b levels; anemia; decreased red blood cell count, decreased hemoglobin and decreased hematocrit; increased mean corpuscular volume; increased mean corpuscular hemoglobin; decreased mean corpuscular volume; decreased mean corpuscular hemoglobin; increased red blood cell distribution width and mean platelet volume; decreased red blood cell distribution width; skewed ratios of B220med/CD23− and B220+/CD11− low/CD23− cells after peritoneal lavage; increased CD25 T cells in lymph node and spleen; increased CD38 non-lymphoid cells in Peyer's patches; increased CD23 B cells (peritoneal); decreased percentage of CD4/CD8 DP cells and increased percentage of TCRB+ cells in thymus; decrease in Peyer's patch B cells; reduced number of TCRB+ CD38+ activated T cells in Peyer's patches; increased splenic CD25+ cells and peritoneal CD23 B cells; increased mean platelet count; decreased mean platelet count; decreased mean serum IgG1 response to an ovalbumin challenge; decreased mean serum IgG2a response to an ovalbumin challenge; increased mean serum IgG2a response to an ovalbumin challenge; increased mean serum MCP-1 response to a LPS challenge; increased mean serum TNF-alpha response to a LPS challenge; increased mean serum IL-6 response to a LPS challenge; increased skin fibroblast proliferation; decreased skin fibroblast proliferation; increased mean percent of total body fat and total fat mass; increased mean body weight; increased mean body length; increased total tissue mass (TTM); increased lean body mass (LBM); increased femoral bone mineral density (BMD); increased vertebral bone mineral density (BMD); increased BMC/LBM ratio; increased bone mineral density (BMD); increased total body volumetric bone mineral density (vBMD); increased bone mineral content (BMC); increased mean femoral midshaft cortical thickness and cross-sectional area; increased mean vertebral trabecular bone volume, number and connectivity density; decreased mean percent of total body fat and total fat mass; decreased mean body weight; decreased mean body length; decreased total tissue mass (TTM); decreased lean body mass (LBM); decreased femoral bone mineral density (BMD); decreased vertebral bone mineral density (BMD); decreased BMC/LBM ratio; decreased bone mineral density (BMD); decreased bone mineral content (BMC); decreased volumetric bone mineral density (vBMD); decreased mean femoral midshaft cortical thickness and cross-sectional area; decreased mean vertebral trabecular bone volume, number and connectivity density; myeloid hyperplasia in bone marrow; osteopetrosis with increased bone mineralization; increase in abdominal fat depots; chronic-active arthritis; proliferative chondrapathy and arthropathy; proliferation of cartilage in femoral tibia joints; chondrous metaplasia of cruciate ligaments and perichondral connective tissues; chronic active dermatitis; chronic active inflammation in periarticular tissues; chronic inflammation in various tissues; myeloid hyperplasia in femur and sternum with associated erythroid hyperplasia in the spleen; increased spleen weight; impaired gastrointestinal motility; thymic atrophy; thymic T cell lymphoma; growth retardation; development abnormalities; stunted growth with general reduction in all organ size; growth retardation with reduced viability; and embryonic lethality.
52. An agent identified by the method of claim 50.
53. The agent of claim 52 which is an agonist or antagonist of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide.
54. The agent of claim 53, wherein the agonist is an anti-PRO179, anti-PRO181, anti-PRO244, anti-PRO247,
anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody.
55. The agent of claim 53, wherein the antagonist is an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody.
56. A method of identifying an agent which modulates a behavior associated with a disruption of the gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising:

(a) providing a non-human transgenic animal whose genome comprises a disruption of the gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide;

(b) observing the behavior exhibited by the non-human transgenic animal of (a);

(c) comparing the observed behavior of (b) with that of a gender matched wild-type animal, wherein the observed behavior exhibited by the non-human transgenic animal that differs from the observed behavior exhibited by the wild-type animal is identified as a behavior associated with gene disruption;

(d) administering a test agent to the non-human transgenic animal of (a); and

(e) determining whether the agent modulates the behavior associated with gene disruption.

57. The method of claim 56, wherein the behavior is an increased anxiety-like response during open field activity testing.
58. The method of claim 56, wherein the behavior is a decreased anxiety-like response during open field activity testing.
59. The method of claim 56, wherein the behavior is an abnormal circadian rhythm during home-cage activity testing.
60. The method of claim 56, wherein the behavior is an enhanced motor coordination during inverted screen testing.
61. The method of claim 56, wherein the behavior is an impaired motor coordination during inverted screen testing.
62. The method of claim 56, wherein the behavior is depression, generalized anxiety disorders, attention deficit disorder, sleep disorder, hyperactivity disorder, obsessive compulsive disorder, schizophrenia, cognitive disorders, hyperalgesia or sensory disorders.
63. An agent identified by the method of claim 56.
64. The agent of claim 63 which is an agonist or antagonist of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide.
65. The agent of claim 64, wherein the agonist is an anti-PRO179, anti-PRO181, anti-PRO244, anti-PRO247,
anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody.
66. The agent of claim 64, wherein the antagonist is an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody.
67. A method of identifying an agent that ameliorates or modulates a neurologic al disorder; a cardiovascular, endothelial or angiogenic disorder; an eye abnormality; an immunological disorder; an oncological disorder; a bone metabolic abnormality or disorder; a lipid metabolic disorder; or a developmental abnormality associated with a disruption in the gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising:

(a) providing a non-human transgenic animal whose genome comprises a disruption of the gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide;

(b) administering a test agent to said non-human transgenic animal; and

(c) determining whether said test agent ameliorates or modulates the neurological disorder; cardiovascular, endothelial or angiogenic disorder; eye abnormality; immunological disorder; oncological disorder; bone metabolic abnormality or disorder; lipid metabolic disorder; or developmental abnormality in the non-human transgenic animal.

68. The method of claim 67, wherein the neurological disorder is an increased anxiety-like response during open field activity testing.
69. The method of claim 67, wherein the neurological disorder is a decreased anxiety-like response during open field activity testing.
70. The method of claim 67, wherein the neurological disorder is an abnormal circadian rhythm during home-cage activity testing.
71. The method of claim 67, wherein the neurological disorder is an enhanced motor coordination during inverted screen testing.
72. The method of claim 67, wherein the neurological disorder is an impaired motor coordination during inverted screen testing.
73. The method of claim 73, wherein the neurological disorder is depression, generalized anxiety disorders, attention deficit disorder, sleep disorder, hyperactivity disorder, obsessive compulsive disorder, schizophrenia, cognitive disorders, hyperalgesia or sensory disorders.
74. The method of claim 67, wherein the eye abnormality is a retinal abnormality.
75. The method of claim 67, wherein the eye abnormality is consistent with vision problems or blindness.
76. The method of claim 74, wherein the retinal abnormality is consistent with retinitis pigmentosa.
77. The method of claim 74, wherein the retinal abnormality is characterized by retinal degeneration or retinal dysplasia.
78. The method of claim 74, wherein the retinal abnormality is consistent with retinal dysplasia, various retinopathies, including retinopathy of prematurity, retrolental fibroplasia, neovascular glaucoma, age-related macular degeneration, diabetic macular edema, corneal neovascularization, corneal graft neovascularization, corneal graft rejection, retinal/choroidal neovascularization, neovascularization of the angle (rubeosis), ocular neovascular disease, vascular restenosis, arteriovenous malformations (AVM), meningioma, hemangioma, angiofibroma, thyroid hyperplasias (including Grave's disease), corneal and other tissue transplantation, retinal artery obstruction or occlusion; retinal degeneration causing secondary atrophy of the retinal vasculature, retinitis pigmentosa, macular dystrophies, Stargardt's disease, congenital stationary night blindness, choroideremia, gyrate atrophy, Leber's congenital amaurosis, retinoschisis disorders, Wagner's syndrome, Usher syndromes, Zellweger syndrome, Saldino-Mainzer syndrome, Senior-Loken syndrome, Bardet-Biedl syndrome, Alport's syndrome, Alstrom's syndrome, Cockayne's syndrome, dysplaisa spondyloepiphysaria congentia, Flynn-Aird syndrome, Friedreich ataxia, Hallgren syndrome, Marshall syndrome, Albers-Schnoberg disease, Refsum's disease, Kearns-Sayre syndrome, Waardenburg's syndrome, Alagile syndrome, myotonic dystrophy, olivopontocerebellar atrophy, Pierre-Marie dunsdrome, Stickler syndrome, carotinemeia, cystinosis, Wolfram syndrome, Bassen-Kornzweig syndrome, abetalipoproteinemia, incontinentia pigmenti, Batten's disease, mucopolysaccharidoses, homocystinuria, or mannosidosis.
79. The method of claim 67, wherein the eye abnormality is a cataract.
80. The method of claim 79, wherein the cataract is a systemic disease such as human Down's syndrome, Hallerman-Streiff syndrome, Lowe syndrome, galactosemia, Marfan syndrome, Trismoy 13-15, Alport syndrome, myotonic dystrophy, Fabry disease, hypoparathroidism or Conradi syndrome.
81. The method of claim 67, wherein the developmental abnormality comprises embryonic lethality or reduced viability.
82. The method of claim 67, wherein the cardiovascular, endothelial or angiogenic disorders are arterial diseases, such as diabetes mellitus; papilledema; optic atrophy; atherosclerosis; angina; myocardial infarctions such as acute myocardial infarctions, cardiac hypertrophy, and heart failure such as congestive heart failure; hypertension; inflammatory vasculitides; Reynaud's disease and Reynaud's phenomenon; aneurysms and arterial restenosis; venous and lymphatic disorders such as thrombophlebitis, lymphangitis, and lymphedema; peripheral vascular disease; cancer such as vascular tumors, e.g., hemangioma (capillary and cavernous), glomus tumors, telangiectasia, bacillary angiomatosis, hemangioendothelioma, angiosarcoma, haemangiopericytoma, Kaposi's sarcoma, lymphangioma, and lymphangiosarcoma; tumor angiogenesis; trauma such as wounds, burns, and other injured tissue, implant fixation, scarring; ischemiareperfusion injury; rheumatoid arthritis; cerebrovascular disease; renal diseases such as acute renal failure, or osteoporosis.
83. The method of claim 67, wherein the immunological disorders are systemic lupus erythematosis; rheumatoid arthritis; juvenile chronic arthritis; spondyloarthropathies; systemic sclerosis (scleroderma); idiopathic inflammatory myopathies (dermatomyositis, polymyositis); Sjögren's syndrome; systemic vasculitis; sarcoidosis; autoimmune hemolytic anemia (immune pancytopenia, paroxysmal nocturnal hemoglobinuria); autoimmune thrombocytopenia (idiopathic thrombocytopenic purpura, immune-mediated thrombocytopenia); thyroiditis (Grave's disease, Hashimoto's thyroiditis, juvenile lymphocytic thyroiditis, atrophic thyroiditis); diabetes mellitus; immune-mediated renal disease (glomerulonephritis, tubulointerstitial nephritis); demyelinating diseases of the central and peripheral nervous systems such as multiple sclerosis, idiopathic demyelinating polyneuropathy or Guillain-Barré syndrome, and chronic inflammatory demyelinating polyneuropathy; hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis; inflammatory bowel disease (ulcerative colitis: Crohn's disease); gluten-sensitive enteropathy, and Whipple's disease; autoimmune or immune-mediated skin diseases including bullous skin diseases, erythema multiforme and contact dermatitis, psoriasis; allergic diseases such as asthma, allergic rhinitis, atopic dermatitis, food hypersensitivity and urticaria; immunologic diseases of the lung such as eosinophilic pneumonia, idiopathic pulmonary fibrosis and hypersensitivity pneumonitis; or transplantation associated diseases including graft rejection and graft-versus-host disease.
84. The method of claim 67, wherein said bone metabolic abnormality or disorder is arthritis, osteoporosis or osteopetrosis.
85. The method of claim 67, wherein the non-human transgenic animal exhibits at least one of the following physiological characteristics compared with gender matched wild-type littermates: increased anxiety-like response during open field testing; decreased anxiety-like response during open field activity testing; hyperactivity with increased rearing and hole poke activity during open field testing; hypoactivity with decreased rearing and hole poke activity during open field testing; increased exploratory activity during open-field testing; decreased exploratory activity during open-field testing; augmentation of circadian rhythm; abnormal circadian rhythm during home-cage activity testing including decreased ambulatory counts; abnormal circadian rhythm during home-cage activity testing including increased ambulatory counts; enhanced circadian rhythm; increased stress induced hyperthermia with increased stress response; increased resistance to stress induced hyperthermia; decreased resistance to stress induced hyperthermia; impaired motor coordination during inverted screen testing; increased depressive-like response during tail suspension testing; decreased depressive-like response during tail suspension testing; decreased startle response during prepulse inhibition testing; no startle response indicating deafness; reduced latency to respond in hot plate testing; increased pain perception in hot plate testing; prolonged latency to respond in hot plate testing; decreased pain perception in hot plate testing; straub tails during functional observational battery testing; opthamological abnormalities; attenuated retinal arteries; optic nerve abnormalities; retinal degeneration; retinal depigmentation; cataracts; decreased heart rate; decreased mean systolic blood pressure; increased mean systolic blood pressure; increased insulin sensitivity; increased mean fasting serum glucose levels; decreased mean serum glucose levels; increased mean serum cholesterol levels; decreased mean serum cholesterol levels; increased mean serum triglyceride levels; decreased mean serum triglyceride levels; enhanced glucose tolerance; impaired glucose tolerance; decreased mean serum insulin levels; increased uric acid levels; ketonemia; increased mean serum phosphorous levels; increased mean serum potassium levels; increased mean serum alkaline phosphatase levels; decreased mean serum alkaline phosphatase levels; blood in the urine; increased nitrituria; ketonuria; decreased mean serum albumin; decreased mean percentage of natural killer cells; abnormal leukocyte count; increased mean percentage of CD4 cells; decreased mean percentage of CD4 cells; increased mean percentage of B cells in peripheral blood; increase in CD4+ and CD8+ cells with decrease in B cells; decreased B cells and less CD11 blow cells in peritoneum; increased mean percentage B cells in spleen, lymph nodes and Peyer's patches; increase in activated/memory T cells by CD25+ staining and CD62L/CD44 staining; increase in activated/memory T cells in spleen; decreased mean percentage of CD8+ cells; increase total white blood cells (increase in neutrophils, lymphocytes, monocytes and basophils); decreased lymphocytes; increased mean absolute monocyte count; increased mean absolute neutrophil count; decreased mean absolute monocyte count; decreased mean serum IgM, IgA, IgG3, IgG2b and IgG2a levels; decreased mean serum IgG3 levels; decreased mean serum IgM levels; decreased mean serum IgG2a levels; decreased mean serum IgG3 and IgM levels; increase in mean serum IgM levels; increase in mean serum IgG2a levels; increase in mean serum IgG2b levels; anemia; decreased red blood cell count, decreased hemoglobin and decreased hematocrit; increased mean corpuscular volume; increased mean corpuscular hemoglobin; decreased mean corpuscular volume; decreased mean corpuscular hemoglobin; increased red blood cell distribution width and mean platelet volume; decreased red blood cell distribution width; skewed ratios of B220med/CD23− and B220+/CD11− low/CD23− cells after peritoneal lavage; increased CD25 T cells in lymph node and spleen; increased CD38 non-lymphoid cells in Peyer's patches; increased CD23 B cells (peritoneal); decreased percentage of CD4/CD8 DP cells and increased percentage of TCRB+ cells in thymus; decrease in Peyer's patch B cells; reduced number of TCRB+ CD38+ activated T cells in Peyer's patches; increased splenic CD25+ cells and peritoneal CD23 B cells; increased mean platelet count; decreased mean platelet count; decreased mean serum IgG1 response to an ovalbumin challenge; decreased mean serum IgG2a response to an ovalbumin challenge; increased mean serum IgG2a response to an ovalbumin challenge; increased mean serum MCP-1 response to a LPS challenge; increased mean serum TNF-alpha response to a LPS challenge; increased mean serum IL-6 response to a LPS challenge; increased skin fibroblast proliferation; decreased skin fibroblast proliferation; increased mean percent of total body fat and total fat mass; increased mean body weight; increased mean body length; increased total tissue mass (TTM); increased lean body mass (LBM); increased femoral bone mineral density (BMD); increased vertebral bone mineral density (BMD); increased BMC/LBM ratio; increased bone mineral density (BMD); increased total body volumetric bone mineral density (vBMD); increased bone mineral content (BMC); increased mean femoral midshaft cortical thickness and cross-sectional area; increased mean vertebral trabecular bone volume, number and connectivity density; decreased mean percent of total body fat and total fat mass; decreased mean body weight; decreased mean body length; decreased total tissue mass (TTM); decreased lean body mass (LBM); decreased femoral bone mineral density (BMD); decreased vertebral bone mineral density (BMD); decreased BMC/LBM ratio; decreased bone mineral density (BMD); decreased bone mineral content (BMC); decreased volumetric bone mineral density (vBMD); decreased mean femoral midshaft cortical thickness and cross-sectional area; decreased mean vertebral trabecular bone volume, number and connectivity density; myeloid hyperplasia in bone marrow; osteopetrosis with increased bone mineralization; increase in abdominal fat depots; chronic-active arthritis; proliferative chondrapathy and arthropathy; proliferation of cartilage in femoral tibia joints; chondrous metaplasia of cruciate ligaments and perichondral connective tissues; chronic active dermatitis; chronic active inflammation in periarticular tissues; chronic inflammation in various tissues; myeloid hyperplasia in femur and sternum with associated erythroid hyperplasia in the spleen; increased spleen weight; impaired gastrointestinal motility; thymic atrophy; thymic T cell lymphoma; growth retardation; development abnormalities; stunted growth with general reduction in all organ size; growth retardation with reduced viability; and embryonic lethality.
86. An agent identified by the method of claim 67.
87. The agent of claim 86 which is an agonist or antagonist of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide.
88. The agent of claim 87, wherein the agonist is an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody.
89. The agent of claim 87, wherein the antagonist is an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody.
90. A therapeutic agent identified by the method of claim 67.
91. A method of identifying an agent that modulates the expression of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising:

(a) contacting a test agent with a host cell expressing a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide; and

(b) determining whether the test agent modulates the expression of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide by the host cell.

92. An agent identified by the method of claim 91.
93. The agent of claim 92 which is an agonist or antagonist of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide.
94. The agent of claim 93, wherein the agonist is an anti-PRO179, anti-PRO181, anti-PRO244, anti-PRO247,
anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody.
95. The agent of claim 93, wherein the antagonist is an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody.
96. A method of evaluating a therapeutic agent capable of affecting a condition associated with a disruption of a gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising:

(a) providing a non-human transgenic animal whose genome comprises a disruption of the gene which encodes for the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide;

(b) measuring a physiological characteristic of the non-human transgenic animal of (a);

(c) comparing the measured physiological characteristic of (b) with that of a gender matched wild-type animal, wherein the physiological characteristic of the non-human transgenic animal that differs from the physiological characteristic of the wild-type animal is identified as a condition resulting from the gene disruption in the non-human transgenic animal;

(d) administering a test agent to the non-human transgenic animal of (a); and

(e) evaluating the effects of the test agent on the identified condition associated with gene disruption in the non-human transgenic animal.

97. The method of claim 96, wherein the condition is a neurological disorder; a cardiovascular, endothelial or angiogenic disorder; an eye abnormality; an immunological disorder; an oncological disorder; a bone metabolic abnormality or disorder; a lipid metabolic disorder; or a developmental abnormality.
98. A therapeutic agent identified by the method of claim 96.
99. The therapeutic agent of claim 98 which is an agonist or antagonist of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide.
100. The therapeutic agent of claim 99, wherein the agonist is an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody.
101. The therapeutic agent of claim 99, wherein the antagonist is an anti-PRO179, anti-PRO181, anti-PRO244,
anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody.
102. A pharmaceutical composition comprising the therapeutic agent of claim 98.
103. A method of treating or preventing or ameliorating a neurological disorder; cardiovascular, endothelial or angiogenic disorder; immunological disorder; oncological disorder; bone metabolic abnormality or disorder, or embryonic lethality associated with the disruption of a gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising administering to a subject in need of such treatment whom may already have the disorder, or may be prone to have the disorder or may be in whom the disorder is to be prevented, a therapeutically effective amount of the therapeutic agent of claim 94, or agonists or antagonists thereof, thereby effectively treating or preventing or ameliorating said disorder.
104. The method of claim 103, wherein the neurological disorder is an increased anxiety-like response during open field activity testing.
105. The method of claim 103, wherein the neurological disorder is a decreased anxiety-like response during open field activity testing.
106. The method of claim 103, wherein the neurological disorder is an abnormal circadian rhythm during home-cage activity testing.
107. The method of claim 103, wherein the neurological disorder is an enhanced motor coordination during inverted screen testing.
108. The method of claim 103, wherein the neurological disorder is an impaired motor coordination during inverted screen testing.
109. The method of claim 103, wherein the neurological disorder is depression, generalized anxiety disorders, attention deficit disorder, sleep disorder, hyperactivity disorder, obsessive compulsive disorder, schizophrenia, cognitive disorders, hyperalgesia or sensory disorders.
110. The method of claim 103, wherein the eye abnormality is a retinal abnormality.
111. The method of claim 103, wherein the eye abnormality is consistent with vision problems or blindness.
112. The method of claim 110, wherein the retinal abnormality is consistent with retinitis pigmentosa.
113. The method of claim 110, wherein the retinal abnormality is characterized by retinal degeneration or retinal dysplasia.
114. The method of claim 110, wherein the retinal abnormality is consistent with retinal dysplasia, various retinopathies, including retinopathy of prematurity, retrolental fibroplasia, neovascular glaucoma, age-related macular degeneration, diabetic macular edema, corneal neovascularization, corneal graft neovascularization, corneal graft rejection, retinal/choroidal neovascularization, neovascularization of the angle (rubeosis), ocular neovascular disease, vascular restenosis, arteriovenous malformations (AVM), meningioma, hemangioma, angiofibroma, thyroid hyperplasias (including Grave's disease), corneal and other tissue transplantation, retinal artery obstruction or occlusion; retinal degeneration causing secondary atrophy of the retinal vasculature, retinitis pigmentosa, macular dystrophies, Stargardt's disease, congenital stationary night blindness, choroideremia, gyrate atrophy, Leber's congenital amaurosis, retinoschisis disorders, Wagner's syndrome, Usher syndromes, Zellweger syndrome, Saldino-Mainzer syndrome, Senior-Loken syndrome, Bardet-Biedl syndrome, Alport's syndrome, Alstrom's syndrome, Cockayne's syndrome, dysplaisa spondyloepiphysaria congentia, Flynn-Aird syndrome, Friedreich ataxia, Hallgren syndrome, Marshall syndrome, Albers-Schnoberg disease, Refsum's disease, Kearns-Sayre syndrome, Waardenburg's syndrome, Alagile syndrome, myotonic dystrophy, olivopontocerebellar atrophy, Pierre-Marie dunsdrome, Stickler syndrome, carotinemeia, cystinosis, Wolfram syndrome, Bassen-Kornzweig syndrome, abetalipoproteinemia, incontinentia pigmenti, Batten's disease, mucopolysaccharidoses, homocystinuria, or mannosidosis.
115. The method of claim 103, wherein the eye abnormality is a cataract.
116. The method of claim 115, wherein the cataract is a systemic disease such as human Down's syndrome, Hallerman-Streiff syndrome, Lowe syndrome, galactosemia, Marfan syndrome, Trismoy 13-15, Alport syndrome, myotonic dystrophy, Fabry disease, hypoparathroidism or Conradi syndrome.
117. The method of claim 103, wherein the developmental abnormality comprises embryonic lethality or reduced viability.
118. The method of claim 103, wherein the cardiovascular, endothelial or angiogenic disorders are arterial diseases, such as diabetes mellitus; papilledema; optic atrophy; atherosclerosis; angina; myocardial infarctions such as acute myocardial infarctions, cardiac hypertrophy, and heart failure such as congestive heart failure; hypertension; inflammatory vasculitides; Reynaud's disease and Reynaud's phenomenon; aneurysms and arterial restenosis; venous and lymphatic disorders such as thrombophlebitis, lymphangitis, and lymphedema; peripheral vascular disease; cancer such as vascular tumors, e.g., hemangioma (capillary and cavernous), glomus tumors, telangiectasia, bacillary angiomatosis, hemangioendothelioma, angiosarcoma, haemangiopericytoma, Kaposi's sarcoma, lymphangioma, and lymphangiosarcoma; tumor angiogenesis; trauma such as wounds, burns, and other injured tissue, implant fixation, scarring; ischemia reperfusion injury; rheumatoid arthritis; cerebrovascular disease; renal diseases such as acute renal failure, or osteoporosis.
119. The method of claim 103, wherein the immunological disorders are systemic lupus erythematosis; rheumatoid arthritis; juvenile chronic arthritis; spondyloarthropathies; systemic sclerosis (scleroderma); idiopathic inflammatory myopathies (dermatomyositis, polymyositis); Sjögren's syndrome; systemic vasculitis; sarcoidosis; autoimmune hemolytic anemia (immune pancytopenia, paroxysmal nocturnal hemoglobinuria); autoimmune thrombocytopenia (idiopathic thrombocytopenic purpura, immune-mediated thrombocytopenia); thyroiditis (Grave's disease, Hashimoto's thyroiditis, juvenile lymphocytic thyroiditis, atrophic thyroiditis); diabetes mellitus; immune-mediated renal disease (glomerulonephritis, tubulointerstitial nephritis); demyelinating diseases of the central and peripheral nervous systems such as multiple sclerosis, idiopathic demyelinating polyneuropathy or Guillain-Barré syndrome, and chronic inflammatory demyelinating polyneuropathy; hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis; inflammatory bowel disease (ulcerative colitis: Crohn's disease); gluten-sensitive enteropathy, and Whipple's disease; autoimmune or immune-mediated skin diseases including bullous skin diseases, erythema multiforme and contact dermatitis, psoriasis; allergic diseases such as asthma, allergic rhinitis, atopic dermatitis, food hypersensitivity and urticaria; immunologic diseases of the lung such as eosinophilic pneumonia, idiopathic pulmonary fibrosis and hypersensitivity pneumonitis; or transplantation associated diseases including graft rejection and graft-versus-host disease.
120. The method of claim 103, wherein said bone metabolic abnormality or disorder is arthritis, osteoporosis or osteopetrosis.
121. A method of identifying an agent that ameliorates or modulates a neurological disorder; a cardiovascular, endothelial or angiogenic disorder; an eye abnormality; an immunological disorder; an oncological disorder; a bone metabolic abnormality or disorder; a lipid metabolic disorder; or a developmental abnormality associated with a disruption in the gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising:

(a) providing a non-human transgenic animal cell culture, each cell of said culture comprising a disruption of the gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide;

(b) administering a test agent to said cell culture; and

(c) determining whether said test agent ameliorates or modulates the neurological disorder; cardiovascular, endothelial or angiogenic disorder; eye abnormality; immunological disorder; oncological disorder; bone metabolic abnormality or disorder; lipid metabolic disorder; or developmental abnormality in said cell culture.

122. The method of claim 121, wherein the neurological disorder is an increased anxiety-like response during open field activity testing.
123. The method of claim 121, wherein the neurological disorder is a decreased anxiety-like response during open field activity testing.
124. The method of claim 121, wherein the neurological disorder is an abnormal circadian rhythm during home-cage activity testing.
125. The method of claim 121, wherein the neurological disorder is an enhanced motor coordination during inverted screen testing.
126. The method of claim 121, wherein the neurological disorder is an impaired motor coordination during inverted screen testing.
127. The method of claim 121, wherein the neurological disorder is depression, generalized anxiety disorders, attention deficit disorder, sleep disorder, hyperactivity disorder, obsessive compulsive disorder, schizophrenia, cognitive disorders, hyperalgesia or sensory disorders.
128. The method of claim 121, wherein the eye abnormality is a retinal abnormality.
129. The method of claim 121, wherein the eye abnormality is consistent with vision problems or blindness.
130. The method of claim 128, wherein the retinal abnormality is consistent with retinitis pigmentosa.
131. The method of claim 128, wherein the retinal abnormality is characterized by retinal degeneration or retinal dysplasia.
132. The method of claim 128, wherein the retinal abnormality is consistent with retinal dysplasia, various retinopathies, including retinopathy of prematurity, retrolental fibroplasia, neovascular glaucoma, age-related macular degeneration, diabetic macular edema, corneal neovascularization, corneal graft neovascularization, corneal graft rejection, retinal/choroidal neovascularization, neovascularization of the angle (rubeosis), ocular neovascular disease, vascular restenosis, arteriovenous malformations (AVM), meningioma, hemangioma, angiofibroma, thyroid hyperplasias (including Grave's disease), corneal and other tissue transplantation, retinal artery obstruction or occlusion; retinal degeneration causing secondary atrophy of the retinal vasculature, retinitis pigmentosa, macular dystrophies, Stargardt's disease, congenital stationary night blindness, choroideremia, gyrate atrophy, Leber's congenital amaurosis, retinoschisis disorders, Wagner's syndrome, Usher syndromes, Zellweger syndrome, Saldino-Mainzer syndrome, Senior-Loken syndrome, Bardet-Biedl syndrome, Alport's syndrome, Alstrom's syndrome, Cockayne's syndrome, dysplaisa spondyloepiphysaria congentia, Flynn-Aird syndrome, Friedreich ataxia, Hallgren syndrome, Marshall syndrome, Albers-Schnoberg disease, Refsum's disease, Kearns-Sayre syndrome, Waardenburg's syndrome, Alagile syndrome, myotonic dystrophy, olivopontocerebellar atrophy, Pierre-Marie dunsdrome, Stickler syndrome, carotinemeia, cystinosis, Wolfram syndrome, Bassen-Kornzweig syndrome, abetalipoproteinemia, incontinentia pigmenti, Batten's disease, mucopolysaccharidoses, homocystinuria, or mannosidosis.
133. The method of claim 121, wherein the eye abnormality is a cataract.
134. The method of claim 133, wherein the cataract is a systemic disease such as human Down's syndrome, Hallerman-Streiff syndrome, Lowe syndrome, galactosemia, Marfan syndrome, Trismoy 13-15, Alport syndrome, myotonic dystrophy, Fabry disease, hypoparathroidism or Conradi syndrome.
135. The method of claim 121, wherein the developmental abnormality comprises embryonic lethality or reduced viability.
136. The method of claim 121, wherein the cardiovascular, endothelial or angiogenic disorders are arterial diseases, such as diabetes mellitus; papilledema; optic atrophy; atherosclerosis; angina; myocardial infarctions such as acute myocardial infarctions, cardiac hypertrophy, and heart failure such as congestive heart failure; hypertension; inflammatory vasculitides; Reynaud's disease and Reynaud's phenomenon; aneurysms and arterial restenosis; venous and lymphatic disorders such as thrombophlebitis, lymphangitis, and lymphedema; peripheral vascular disease; cancer such as vascular tumors, e.g., hemangioma (capillary and cavernous), glomus tumors, telangiectasia, bacillary angiomatosis, hemangioendothelioma, angiosarcoma, haemangiopericytoma, Kaposi's sarcoma, lymphangioma, and lymphangiosarcoma; tumor angiogenesis; trauma such as wounds, burns, and other injured tissue, implant fixation, scarring; ischemia reperfusion injury; rheumatoid arthritis; cerebrovascular disease; renal diseases such as acute renal failure, or osteoporosis.
137. The method of claim 121, wherein the immunological disorders are systemic lupus erythematosis; rheumatoid arthritis; juvenile chronic arthritis; spondyloarthropathies; systemic sclerosis (scleroderma); idiopathic inflammatory myopathies (dermatomyositis, polymyositis); Sjögren's syndrome; systemic vasculitis; sarcoidosis; autoimmune hemolytic anemia (immune pancytopenia, paroxysmal nocturnal hemoglobinuria); autoimmune thrombocytopenia (idiopathic thrombocytopenic purpura, immune-mediated thrombocytopenia); thyroiditis (Grave's disease, Hashimoto's thyroiditis, juvenile lymphocytic thyroiditis, atrophic thyroiditis); diabetes mellitus; immune-mediated renal disease (glomerulonephritis, tubulointerstitial nephritis); demyelinating diseases of the central and peripheral nervous systems such as multiple sclerosis, idiopathic demyelinating polyneuropathy or Guillain-Barré syndrome, and chronic inflammatory demyelinating polyneuropathy; hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis; inflammatory bowel disease (ulcerative colitis: Crohn's disease); gluten-sensitive enteropathy, and Whipple's disease; autoimmune or immune-mediated skin diseases including bullous skin diseases, erythema multiforme and contact dermatitis, psoriasis; allergic diseases such as asthma, allergic rhinitis, atopic dermatitis, food hypersensitivity and urticaria; immunologic diseases of the lung such as eosinophilic pneumonia, idiopathic pulmonary fibrosis and hypersensitivity pneumonitis; or transplantation associated diseases including graft rejection and graft-versus-host disease.
138. The method of claim 121, wherein said bone metabolic abnormality or disorder is arthritis, osteoporosis or osteopetrosis.
139. An agent identified by the method of claim 121.
140. The agent of claim 139 which is an agonist or antagonist of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide.
141. The agent of claim 140, wherein the agonist is an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody.
142. The agent of claim 140, wherein the antagonist is an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody.
143. A therapeutic agent identified by the method of claim 121.
144. A method of modulating a phenotype associated with a disruption of a gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising administering to a subject whom may already have the phenotype, or may be prone to have the phenotype or may be in whom the phenotype is to be prevented, an effective amount of the agent of claim 46, or agonists or antagonists thereof, thereby effectively modulating the phenotype.
145. A method of modulating a physiological characteristic associated with a disruption of a gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising administering to a subject whom may already exhibit the physiological characteristic, or may be prone to exhibit the physiological characteristic or may be in whom the physiological characteristic is to be prevented, an effective amount of the agent of claim 52, or agonists or antagonists thereof, thereby effectively modulating the physiological characteristic.
146. A method of modulating a behavior associated with a disruption of a gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising administering to a subject whom may already exhibit the behavior, or may be prone to exhibit the behavior or may be in whom the exhibited behavior is to be prevented, an effective amount of the agent of claim 63, or agonists or antagonists thereof, thereby effectively modulating the behavior.
147. A method of modulating the expression of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising administering to a host cell expressing said PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, an effective amount of the agent of claim 92, or agonists or antagonists thereof, thereby effectively modulating the expression of said polypeptide.
148. A method of modulating a condition associated with a disruption of a gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising administering to a subject whom may have the condition, or may be prone to have the condition or may be in whom the condition is to be prevented, a therapeutically effective amount of the therapeutic agent of claim 98, or agonists or antagonists thereof, thereby effectively modulating the condition.
149. A method of treating or preventing or ameliorating a neurological disorder; cardiovascular, endothelial or angiogenic disorder; immunological disorder; oncological disorder; bone metabolic abnormality or disorder, or embryonic lethality associated with the disruption of a gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising administering to a non-human transgenic animal cell culture, each cell of said culture comprising a disruption of the gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, a therapeutically effective amount of the agent of claim 139, or agonists or antagonists thereof, thereby effectively treating or preventing or ameliorating said disorder.

### BRIEF DESCRIPTION OF THE DRAWINGS

FIG. 1 shows a nucleotide sequence (SEQ ID NO:1) of a native sequence PRO179 cDNA, wherein SEQ ID NO:1 is a clone designated herein as “DNA16451-1078” (UNQ153).

FIG. 2 shows the amino acid sequence (SEQ ID NO:2) derived from the coding sequence of SEQ ID NO:1 shown in FIG. 1.

FIG. 3 shows a nucleotide sequence (SEQ ID NO:3) of a native sequence PRO181 cDNA, wherein SEQ ID NO:3 is a clone designated herein as “DNA23330-1390” (UNQ155).

FIG. 4 shows the amino acid sequence (SEQ ID NO:4) derived from the coding sequence of SEQ ID NO:3 shown in FIG. 3.

FIG. 5 shows a nucleotide sequence (SEQ ID NO:5) of a native sequence PRO244 cDNA, wherein SEQ ID NO:5 is a clone designated herein as “DNA35668-1171” (UNQ218).

FIG. 6 shows the amino acid sequence (SEQ ID NO:6) derived from the coding sequence of SEQ ID NO:5 shown in FIG. 5.

FIG. 7 shows a nucleotide sequence (SEQ ID NO:7) of a native sequence PRO247 cDNA, wherein SEQ ID NO:7 is a clone designated herein as “DNA35673-1201” (UNQ221).

FIG. 8 shows the amino acid sequence (SEQ ID NO:8) derived from the coding sequence of SEQ ID NO:7 shown in FIG. 7.

FIG. 9 shows a nucleotide sequence (SEQ ID NO:9) of a native sequence PRO269 cDNA, wherein SEQ ID NO:9 is a clone designated herein as “DNA38260-1180” (UNQ236).

FIG. 10 shows the amino acid sequence (SEQ ID NO:10) derived from the coding sequence of SEQ ID NO:9 shown in FIG. 9.

FIG. 11 shows a nucleotide sequence (SEQ ID NO:11) of a native sequence PRO293 cDNA, wherein SEQ ID NO:11 is a clone designated herein as “DNA37151-1193” (UNQ256).

FIG. 12 shows the amino acid sequence (SEQ ID NO:12) derived from the coding sequence of SEQ ID NO:11 shown in FIG. 11.

FIG. 13 shows a nucleotide sequence (SEQ ID NO:13) of a native sequence PRO298 cDNA, wherein SEQ ID NO:13 is a clone designated herein as “DNA39975-1210” (UNQ261).

FIG. 14 shows the amino acid sequence (SEQ ID NO:14) derived from the coding sequence of SEQ ID NO:13 shown in FIG. 13.

FIG. 15 shows a nucleotide sequence (SEQ ID NO:15) of a native sequence PRO339 cDNA, wherein SEQ ID NO:15 is a clone designated herein as “DNA43466-1225” (UNQ299).

FIG. 16 shows the amino acid sequence (SEQ ID NO:16) derived from the coding sequence of SEQ ID NO:15 shown in FIG. 15.

FIG. 17 shows a nucleotide sequence (SEQ ID NO:17) of a native sequence PRO341 cDNA, wherein SEQ ID NO:17 is a clone designated herein as “DNA26288-1239” (UNQ300).

FIG. 18 shows the amino acid sequence (SEQ ID NO:18) derived from the coding sequence of SEQ ID NO:17 shown in FIG. 17.

FIG. 19 shows a nucleotide sequence (SEQ ID NO:19) of a native sequence PRO347 cDNA, wherein SEQ ID NO:19 is a clone designated herein as “DNA44176-1244” (UNQ306).

FIG. 20 shows the amino acid sequence (SEQ ID NO:20) derived from the coding sequence of SEQ ID NO:19 shown in FIG. 19.

FIG. 21 shows a nucleotide sequence (SEQ ID NO:21) of a native sequence PRO531 cDNA, wherein SEQ ID NO:21 is a clone designated herein as “DNA48314-1320” (UNQ332).

FIG. 22 shows the amino acid sequence (SEQ ID NO:22) derived from the coding sequence of SEQ ID NO:21 shown in FIG. 21.

FIG. 23 shows a nucleotide sequence (SEQ ID NO:23) of a native sequence PRO537 cDNA, wherein SEQ ID NO:23 is a clone designated herein as “DNA49141-1431” (UNQ338).

FIG. 24 shows the amino acid sequence (SEQ ID NO:24) derived from the coding sequence of SEQ ID NO:23 shown in FIG. 23.

FIG. 25 shows a nucleotide sequence (SEQ ID NO:25) of a native sequence PRO718 cDNA, wherein SEQ ID NO:25 is a clone designated herein as “DNA49647-1398” (UNQ386).

FIG. 26 shows the amino acid sequence (SEQ ID NO:26) derived from the coding sequence of SEQ ID NO:25 shown in FIG. 25.

FIG. 27 shows a nucleotide sequence (SEQ ID NO:27) of a native sequence PRO773 cDNA, wherein SEQ ID NO:27 is a clone designated herein as “DNA48303-2829” (UNQ411).

FIG. 28 shows the amino acid sequence (SEQ ID NO:28) derived from the coding sequence of SEQ ID NO:27 shown in FIG. 27.

FIG. 29 shows a nucleotide sequence (SEQ ID NO:29) of a native sequence PRO860 cDNA, wherein SEQ ID NO:29 is a clone designated herein as “DNA60614” (UNQ421).

FIG. 30 shows the amino acid sequence (SEQ ID NO:30) derived from the coding sequence of SEQ ID NO:29 shown in FIG. 29.

FIG. 31 shows a nucleotide sequence (SEQ ID NO:31) of a native sequence PRO871 cDNA, wherein SEQ ID NO:31 is a clone designated herein as “DNA50919-1361” (UNQ438).

FIG. 32 shows the amino acid sequence (SEQ ID NO:32) derived from the coding sequence of SEQ ID NO:31 shown in FIG. 31.

FIG. 33 shows a nucleotide sequence (SEQ ID NO:33) of a native sequence PRO872 cDNA, wherein SEQ ID NO:33 is a clone designated herein as “DNA49819-1439” (UNQ439).

FIG. 34 shows the amino acid sequence (SEQ ID NO:34) derived from the coding sequence of SEQ ID NO:33 shown in FIG. 33.

FIG. 35 shows a nucleotide sequence (SEQ ID NO:35) of a native sequence PRO813 cDNA, wherein SEQ ID NO:35 is a clone designated herein as “DNA57834-1339” (UNQ465).

FIG. 36 shows the amino acid sequence (SEQ ID NO:36) derived from the coding sequence of SEQ ID NO:35 shown in FIG. 35.

FIG. 37 shows a nucleotide sequence (SEQ ID NO:37) of a native sequence PRO828 cDNA, wherein SEQ ID NO:37 is a clone designated herein as “DNA57037-1444” (UNQ469).

FIG. 38 shows the amino acid sequence (SEQ ID NO:38) derived from the coding sequence of SEQ ID NO:37 shown in FIG. 37.

FIG. 39 shows a nucleotide sequence (SEQ ID NO:39) of a native sequence PRO1100 cDNA, wherein SEQ ID NO:39 is a clone designated herein as “DNA59619-1464” (UNQ546).

FIG. 40 shows the amino acid sequence (SEQ ID NO:40) derived from the coding sequence of SEQ ID NO:39 shown in FIG. 39.

FIG. 41 shows a nucleotide sequence (SEQ ID NO:41) of a native sequence PRO1114 cDNA, wherein SEQ ID NO:41 is a clone designated herein as “DNA57033-1403” (UNQ557).

FIG. 42 shows the amino acid sequence (SEQ ID NO:42) derived from the coding sequence of SEQ ID NO:41 shown in FIG. 41.

FIG. 43 shows a nucleotide sequence (SEQ ID NO:43) of a native sequence PROMS cDNA, wherein SEQ ID NO:43 is a clone designated herein as “DNA56868-1478” (UNQ558).

FIG. 44 shows the amino acid sequence (SEQ ID NO:44) derived from the coding sequence of SEQ ID NO:41 shown in FIG. 41.

FIG. 45 shows a nucleotide sequence (SEQ ID NO:45) of a native sequence PRO1126 cDNA, wherein SEQ ID NO:45 is a clone designated herein as “DNA60615-1483” (UNQ564).

FIG. 46 shows the amino acid sequence (SEQ ID NO:46) derived from the coding sequence of SEQ ID NO:45 shown in FIG. 45.

FIG. 47 shows a nucleotide sequence (SEQ ID NO:47) of a native sequence PRO1133 cDNA, wherein SEQ ID NO:47 is a clone designated herein as “DNA53913-1490” (UNQ571).

FIG. 48 shows the amino acid sequence (SEQ ID NO:48) derived from the coding sequence of SEQ ID NO:41 shown in FIG. 41.

FIG. 49 shows a nucleotide sequence (SEQ ID NO:49) of a native sequence PRO1154 cDNA, wherein SEQ ID NO:49 is a clone designated herein as “DNA59846-1503” (UNQ584).

FIG. 50 shows the amino acid sequence (SEQ ID NO:50) derived from the coding sequence of SEQ ID NO:49 shown in FIG. 49.

FIG. 51 shows a nucleotide sequence (SEQ ID NO:51) of a native sequence PRO1185 cDNA, wherein SEQ ID NO:51 is a clone designated herein as “DNA62881-1515” (UNQ599).

FIG. 52 shows the amino acid sequence (SEQ ID NO:52) derived from the coding sequence of SEQ ID NO:51 shown in FIG. 51.

FIG. 53 shows a nucleotide sequence (SEQ ID NO:53) of a native sequence PRO1194 cDNA, wherein SEQ ID NO:53 is a clone designated herein as “DNA57841-1522” (UNQ607).

FIG. 54 shows the amino acid sequence (SEQ ID NO:54) derived from the coding sequence of SEQ ID NO:53 shown in FIG. 53.

FIG. 55 shows a nucleotide sequence (SEQ ID NO:55) of a native sequence PRO1287 cDNA, wherein SEQ ID NO:55 is a clone designated herein as “DNA61755-1554” (UNQ656).

FIG. 56 shows the amino acid sequence (SEQ ID NO:56) derived from the coding sequence of SEQ ID NO:55 shown in FIG. 55.

FIG. 57 shows a nucleotide sequence (SEQ ID NO:57) of a native sequence PRO1291 cDNA, wherein SEQ ID NO:57 is a clone designated herein as “DNA59610-1556” (UNQ659).

FIG. 58 shows the amino acid sequence (SEQ ID NO:58) derived from the coding sequence of SEQ ID NO:57 shown in FIG. 57.

FIG. 59 shows a nucleotide sequence (SEQ ID NO:59) of a native sequence PRO1293 cDNA, wherein SEQ ID NO:59 is a clone designated herein as “DNA60618-1557” (UNQ662).

FIG. 60 shows the amino acid sequence (SEQ ID NO:60) derived from the coding sequence of SEQ ID NO:59 shown in FIG. 59.

FIG. 61 shows a nucleotide sequence (SEQ ID NO:61) of a native sequence PRO1310 cDNA, wherein SEQ ID NO:61 is a clone designated herein as “DNA47394-1572” (UNQ676).

FIG. 62 shows the amino acid sequence (SEQ ID NO:62) derived from the coding sequence of SEQ ID NO:61 shown in FIG. 61.

FIG. 63 shows a nucleotide sequence (SEQ ID NO:63) of a native sequence PRO1312 cDNA, wherein SEQ ID NO:63 is a clone designated herein as “DNA61873-1574” (UNQ678).

FIG. 64 shows the amino acid sequence (SEQ ID NO:64) derived from the coding sequence of SEQ ID NO:63 shown in FIG. 63.

FIG. 65 shows a nucleotide sequence (SEQ ID NO:65) of a native sequence PRO1335 cDNA, wherein SEQ ID NO:65 is a clone designated herein as “DNA62812-1594” (UNQ690).

FIG. 66 shows the amino acid sequence (SEQ ID NO:66) derived from the coding sequence of SEQ ID NO:65 shown in FIG. 65.

FIG. 67 shows a nucleotide sequence (SEQ ID NO:67) of a native sequence PRO1339 cDNA, wherein SEQ ID NO:67 is a clone designated herein as “DNA66669-1597” (UNQ694).

FIG. 68 shows the amino acid sequence (SEQ ID NO:68) derived from the coding sequence of SEQ ID NO:67 shown in FIG. 67.

FIG. 69 shows a nucleotide sequence (SEQ ID NO:69) of a native sequence PRO2155 cDNA, wherein SEQ ID NO:69 is a clone designated herein as “DNA88062” (UNQ696).

FIG. 70 shows the amino acid sequence (SEQ ID NO:70) derived from the coding sequence of SEQ ID NO:69 shown in FIG. 69.

FIG. 71 shows a nucleotide sequence (SEQ ID NO:71) of a native sequence PRO1356 cDNA, wherein SEQ ID NO:71 is a clone designated herein as “DNA64886-1601” (UNQ705).

FIG. 72 shows the amino acid sequence (SEQ ID NO:72) derived from the coding sequence of SEQ ID NO:71 shown in FIG. 71.

FIG. 73 shows a nucleotide sequence (SEQ ID NO:73) of a native sequence PRO1385 cDNA, wherein SEQ ID NO:73 is a clone designated herein as “DNA68869-1610” (UNQ720).

FIG. 74 shows the amino acid sequence (SEQ ID NO:74) derived from the coding sequence of SEQ ID NO:73 shown in FIG. 73.

FIG. 75 shows a nucleotide sequence (SEQ ID NO:75) of a native sequence PRO1412 cDNA, wherein SEQ ID NO:75 is a clone designated herein as “DNA64897-1628” (UNQ730).

FIG. 76 shows the amino acid sequence (SEQ ID NO:76) derived from the coding sequence of SEQ ID NO:75 shown in FIG. 75.

FIG. 77A-77B shows a nucleotide sequence (SEQ ID NO:77) of a native sequence PRO1487 Cdna, wherein SEQ ID NO:77 is a clone designated herein as “DNA68836-1656” (UNQ756).

FIG. 78A-78B shows the amino acid sequence (SEQ ID NO:78) derived from the coding sequence of SEQ ID NO:77 shown in FIG. 77A-77B.

FIG. 79 shows a nucleotide sequence (SEQ ID NO:79) of a native sequence PRO1758 cDNA, wherein SEQ ID NO:79 is a clone designated herein as “DNA76399-1700” (UNQ831).

FIG. 80 shows the amino acid sequence (SEQ ID NO:80) derived from the coding sequence of SEQ ID NO:79 shown in FIG. 79.

FIG. 81 shows a nucleotide sequence (SEQ ID NO:81) of a native sequence PRO1779 cDNA, wherein SEQ ID NO:81 is a clone designated herein as “DNA73775-1707” (UNQ841).

FIG. 82A-82B shows the amino acid sequence (SEQ ID NO:82) derived from the coding sequence of SEQ ID NO:81 shown in FIG. 81.

FIG. 83 shows a nucleotide sequence (SEQ ID NO:83) of a native sequence PRO1785 cDNA, wherein SEQ ID NO:83 is a clone designated herein as “DNA80136-2503” (UNQ847).

FIG. 84 shows the amino acid sequence (SEQ ID NO:84) derived from the coding sequence of SEQ ID NO:83 shown in FIG. 83.

FIG. 85 shows a nucleotide sequence (SEQ ID NO:85) of a native sequence PRO1889 cDNA, wherein SEQ ID NO:85 is a clone designated herein as “DNA77623-2524” (UNQ871).

FIG. 86 shows the amino acid sequence (SEQ ID NO:86) derived from the coding sequence of SEQ ID NO:85 shown in FIG. 85.

FIG. 87A-87B shows a nucleotide sequence (SEQ ID NO:87) of a native sequence PRO90318 Cdna, wherein SEQ ID NO:87 is a clone designated herein as “DNA336109” (UNQ907).

FIG. 88 shows the amino acid sequence (SEQ ID NO:88) derived from the coding sequence of SEQ ID NO:87 shown in FIG. 87A-87B.

FIG. 89A-89B shows a nucleotide sequence (SEQ ID NO:89) of a native sequence PRO3434 cDNA, wherein SEQ ID NO:89 is a clone designated herein as “DNA77631-2537” (UNQ1821).

FIG. 90 shows the amino acid sequence (SEQ ID NO:90) derived from the coding sequence of SEQ ID NO:89 shown in FIG. 89A-89B.

FIG. 91 shows a nucleotide sequence (SEQ ID NO:91) of a native sequence PRO3579 cDNA, wherein SEQ ID NO:91 is a clone designated herein as “DNA68862-2546” (UNQ1849).

FIG. 92 shows the amino acid sequence (SEQ ID NO:92) derived from the coding sequence of SEQ ID NO:91 shown in FIG. 91.

FIG. 93 shows a nucleotide sequence (SEQ ID NO:93) of a native sequence PRO4322 cDNA, wherein SEQ ID NO:93 is a clone designated herein as “DNA92223-2567” (UNQ1879).

FIG. 94 shows the amino acid sequence (SEQ ID NO:94) derived from the coding sequence of SEQ ID NO:93 shown in FIG. 93.

FIG. 95 shows a nucleotide sequence (SEQ ID NO:95) of a native sequence PRO4343 cDNA, wherein SEQ ID NO:95 is a clone designated herein as “DNA92255-2584” (UNQ1897).

FIG. 96 shows the amino acid sequence (SEQ ID NO:96) derived from the coding sequence of SEQ ID NO:95 shown in FIG. 95.

FIG. 97 shows a nucleotide sequence (SEQ ID NO:97) of a native sequence PRO4347 cDNA, wherein SEQ ID NO:97 is a clone designated herein as “DNA92288-2588” (UNQ1901).

FIG. 98 shows the amino acid sequence (SEQ ID NO:98) derived from the coding sequence of SEQ ID NO:97 shown in FIG. 97.

FIG. 99 shows a nucleotide sequence (SEQ ID NO:99) of a native sequence PRO4403 cDNA, wherein SEQ ID NO:99 is a clone designated herein as “DNA83509-2612” (UNQ1928).

FIG. 100 shows the amino acid sequence (SEQ ID NO:100) derived from the coding sequence of SEQ ID NO:99 shown in FIG. 99.

FIG. 101 shows a nucleotide sequence (SEQ ID NO:101) of a native sequence PRO4976 Cdna, wherein SEQ ID NO:101 is a clone designated herein as “DNA100902-2646” (UNQ2419).

FIG. 102 shows the amino acid sequence (SEQ ID NO:102) derived from the coding sequence of SEQ ID NO:101 shown in FIG. 101.

FIG. 103 shows a nucleotide sequence (SEQ ID NO:103) of a native sequence PRO260 cDNA, wherein SEQ ID NO:103 is a clone designated herein as “DNA33470-1175” (UNQ227).

FIG. 104 shows the amino acid sequence (SEQ ID NO:104) derived from the coding sequence of SEQ ID NO:103 shown in FIG. 103.

FIG. 105 shows a nucleotide sequence (SEQ ID NO:105) of a native sequence PRO6014 cDNA, wherein SEQ ID NO:105 is a clone designated herein as “DNA92217-2697” (UNQ2521).

FIG. 106 shows the amino acid sequence (SEQ ID NO:106) derived from the coding sequence of SEQ ID NO:105 shown in FIG. 105.

FIG. 107 shows a nucleotide sequence (SEQ ID NO:107) of a native sequence PRO6027 cDNA, wherein SEQ ID NO:107 is a clone designated herein as “DNA105838-2702” (UNQ2528).

FIG. 108 shows the amino acid sequence (SEQ ID NO:108) derived from the coding sequence of SEQ ID NO:107 shown in FIG. 107.

FIG. 109 shows a nucleotide sequence (SEQ ID NO:109) of a native sequence PRO6181 cDNA, wherein SEQ ID NO:109 is a clone designated herein as “DNA107698-2715” (UNQ2552).

FIG. 110 shows the amino acid sequence (SEQ ID NO:110) derived from the coding sequence of SEQ ID NO:109 shown in FIG. 109.

FIG. 111 shows a nucleotide sequence (SEQ ID NO:111) of a native sequence PRO6714 cDNA, wherein SEQ ID NO:111 is a clone designated herein as “DNA82358-2738” (UNQ2759).

FIG. 112 shows the amino acid sequence (SEQ ID NO:112) derived from the coding sequence of SEQ ID NO:111 shown in FIG. 111.

FIG. 113A-113B shows a nucleotide sequence (SEQ ID NO:113) of a native sequence PRO9922 cDNA, wherein SEQ ID NO:113 is a clone designated herein as “DNA142524” (UNQ2768).

FIG. 114 shows the amino acid sequence (SEQ ID NO:114) derived from the coding sequence of SEQ ID NO:113 shown in FIG. 113A-113B.

FIG. 115 shows a nucleotide sequence (SEQ ID NO:115) of a native sequence PRO7179 cDNA, wherein SEQ ID NO:115 is a clone designated herein as “DNA108701-2749” (UNQ2789).

FIG. 116 shows the amino acid sequence (SEQ ID NO:116) derived from the coding sequence of SEQ ID NO:115 shown in FIG. 115.

FIG. 117 shows a nucleotide sequence (SEQ ID NO:117) of a native sequence PRO7476 cDNA, wherein SEQ ID NO:117 is a clone designated herein as “DNA115253-2757” (UNQ2976).

FIG. 118 shows the amino acid sequence (SEQ ID NO:118) derived from the coding sequence of SEQ ID NO:117 shown in FIG. 117.

FIG. 119A-119B shows a nucleotide sequence (SEQ ID NO:119) of a native sequence PRO9824 cDNA, wherein SEQ ID NO:119 is a clone designated herein as “DNA111030” (UNQ3026).

FIG. 120 shows the amino acid sequence (SEQ ID NO:120) derived from the coding sequence of SEQ ID NO:119 shown in FIG. 119A-119B.

FIG. 121 shows a nucleotide sequence (SEQ ID NO:121) of a native sequence PRO19814 cDNA, wherein SEQ ID NO:121 is a clone designated herein as “DNA148004-2882” (UNQ5923).

FIG. 122 shows the amino acid sequence (SEQ ID NO:122) derived from the coding sequence of SEQ ID NO:121 shown in FIG. 121.

FIG. 123A-123B shows a nucleotide sequence (SEQ ID NO:123) of a native sequence PRO19836 cDNA, wherein SEQ ID NO:123 is a clone designated herein as “DNA144839” (UNQ5930).

FIG. 124 shows the amino acid sequence (SEQ ID NO:124) derived from the coding sequence of SEQ ID NO:123 shown in FIG. 123A-123B.

FIG. 125 shows a nucleotide sequence (SEQ ID NO:125) of a native sequence PRO20088 cDNA, wherein SEQ ID NO:125 is a clone designated herein as “DNA150157-2898” (UNQ6077).

FIG. 126 shows the amino acid sequence (SEQ ID NO:126) derived from the coding sequence of SEQ ID NO:125 shown in FIG. 125.

FIG. 127 shows a nucleotide sequence (SEQ ID NO:127) of a native sequence PRO70789 cDNA, wherein SEQ ID NO:127 is a clone designated herein as “DNA295801” (UNQ9659).

FIG. 128 shows the amino acid sequence (SEQ ID NO:128) derived from the coding sequence of SEQ ID NO:127 shown in FIG. 127.

FIG. 129 shows a nucleotide sequence (SEQ ID NO:129) of a native sequence PRO50298 cDNA, wherein SEQ ID NO:129 is a clone designated herein as “DNA255219” (UNQ11632).

FIG. 130 shows the amino acid sequence (SEQ ID NO:130) derived from the coding sequence of SEQ ID NO:129 shown in FIG. 129.

FIG. 131 shows a nucleotide sequence (SEQ ID NO:131) of a native sequence PRO51592 cDNA, wherein SEQ ID NO:131 is a clone designated herein as “DNA256561” (UNQ12179).

FIG. 132 shows the amino acid sequence (SEQ ID NO:132) derived from the coding sequence of SEQ ID NO:131 shown in FIG. 131.

FIG. 133 shows a nucleotide sequence (SEQ ID NO:133) of a native sequence PRO1757 cDNA, wherein SEQ ID NO:133 is a clone designated herein as “DNA76398-1699” (UNQ830).

FIG. 134 shows the amino acid sequence (SEQ ID NO:134) derived from the coding sequence of SEQ ID NO:133 shown in FIG. 133.

FIG. 135 shows a nucleotide sequence (SEQ ID NO:135) of a native sequence PRO4421 cDNA, wherein SEQ ID NO:135 is a clone designated herein as “DNA96879-2619” (UNQ1938).

FIG. 136 shows the amino acid sequence (SEQ ID NO:136) derived from the coding sequence of SEQ ID NO:135 shown in FIG. 135.

FIG. 137 shows a nucleotide sequence (SEQ ID NO:137) of a native sequence PRO9903 cDNA, wherein SEQ ID NO:137 is a clone designated herein as “DNA119516-2797” (UNQ3071).

FIG. 138 shows the amino acid sequence (SEQ ID NO:138) derived from the coding sequence of SEQ ID NO:137 shown in FIG. 137.

FIG. 139 shows a nucleotide sequence (SEQ ID NO:139) of a native sequence PRO1106 cDNA, wherein SEQ ID NO:139 is a clone designated herein as “DNA59609-1470” (UNQ549).

FIG. 140 shows the amino acid sequence (SEQ ID NO:140) derived from the coding sequence of SEQ ID NO:139 shown in FIG. 139.

FIG. 141 shows a nucleotide sequence (SEQ ID NO:141) of a native sequence PRO1411 cDNA, wherein SEQ ID NO:141 is a clone designated herein as “DNA59212-1627” (UNQ729).

FIG. 142 shows the amino acid sequence (SEQ ID NO:142) derived from the coding sequence of SEQ ID NO:141 shown in FIG. 141.

FIG. 143 shows a nucleotide sequence (SEQ ID NO:143) of a native sequence PRO1486 cDNA, wherein SEQ ID NO:143 is a clone designated herein as “DNA71180-1655” (UNQ755).

FIG. 144 shows the amino acid sequence (SEQ ID NO:144) derived from the coding sequence of SEQ ID NO:143 shown in FIG. 143.

FIG. 145 shows a nucleotide sequence (SEQ ID NO:145) of a native sequence PRO1565 cDNA, wherein SEQ ID NO:145 is a clone designated herein as “DNA73727-1643” (UNQ771).

FIG. 146 shows the amino acid sequence (SEQ ID NO:146) derived from the coding sequence of SEQ ID NO:145 shown in FIG. 145.

FIG. 147 shows a nucleotide sequence (SEQ ID NO:147) of a native sequence PRO4399 cDNA, wherein SEQ ID NO:147 is a clone designated herein as “DNA89220-2609” (UNQ1924).

FIG. 148 shows the amino acid sequence (SEQ ID NO:148) derived from the coding sequence of SEQ ID NO:147 shown in FIG. 147.

FIG. 149 shows a nucleotide sequence (SEQ ID NO:149) of a native sequence PRO4404 cDNA, wherein SEQ ID NO:149 is a clone designated herein as “DNA84142-2613” (UNQ1929).

FIG. 150 shows the amino acid sequence (SEQ ID NO:150) derived from the coding sequence of SEQ ID NO:149 shown in FIG. 149.

### DETAILED DESCRIPTION OF THE PREFERRED EMBODIMENTS

### Definitions

The terms “PRO polypeptide” and “PRO” as used herein and when immediately followed by a numerical designation refer to various polypeptides, wherein the complete designation (i.e., PRO/number) refers to specific polypeptide sequences as described herein. The terms “PRO/number polypeptide” and “PRO/number” wherein the term “number” is provided as an actual numerical designation as used herein encompass native sequence polypeptides and polypeptide variants (which are further defined herein). The PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides described herein may be isolated from a variety of sources, such as from human tissue types or from another source, or prepared by recombinant or synthetic methods. The term “PRO polypeptide” refers to each individual PRO/number polypeptide disclosed herein. All disclosures in this specification which refer to the “PRO polypeptide” refer to each of the polypeptides individually as well as jointly. For example, descriptions of the preparation of, purification of, derivation of, formation of antibodies to or against, administration of, compositions containing, treatment of a disease with, etc., pertain to each polypeptide of the invention individually. The term “PRO polypeptide” also includes variants of the PRO/number polypeptides disclosed herein.

A “native sequence PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide” comprises a polypeptide having the same amino acid sequence as the corresponding PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide derived from nature. Such native sequence PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides can be isolated from nature or can be produced by recombinant or synthetic means. The term “native sequence PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide” specifically encompasses naturally-occurring truncated or secreted forms of the specific PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide (e.g., an extracellular domain sequence), naturally-occurring variant forms (e.g., alternatively spliced forms) and naturally-occurring allelic variants of the polypeptide. The invention provides native sequence PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides disclosed herein which are mature or full-length native sequence polypeptides comprising the full-length amino acids sequences shown in the accompanying figures. Start and stop codons are shown in bold font and underlined in the figures. However, while the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide disclosed in the accompanying figures are shown to begin with methionine residues designated herein as amino acid position 1 in the figures, it is conceivable and possible that other methionine residues located either upstream or downstream from the amino acid position 1 in the figures may be employed as the starting amino acid residue for the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides.

The PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide “extracellular domain” or “ECD” refers to a form of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide which is essentially free of the transmembrane and cytoplasmic domains. Ordinarily, a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide

ECD will have less than 1% of such transmembrane and/or cytoplasmic domains and preferably, will have less than 0.5% of such domains. It will be understood that any transmembrane domains identified for the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides of the present invention are identified pursuant to criteria routinely employed in the art for identifying that type of hydrophobic domain. The exact boundaries of a transmembrane domain may vary but most likely by no more than about 5 amino acids at either end of the domain as initially identified herein. Optionally, therefore, an extracellular domain of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide may contain from about 5 or fewer amino acids on either side of the transmembrane domain/extracellular domain boundary as identified in the Examples or specification and such polypeptides, with or without the associated signal peptide, and nucleic acid encoding them, are contemplated by the present invention.

The approximate location of the “signal peptides” of the various PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides disclosed herein are shown in the present specification and/or the accompanying figures. It is noted, however, that the C-terminal boundary of a signal peptide may vary, but most likely by no more than about 5 amino acids on either side of the signal peptide C-terminal boundary as initially identified herein, wherein the C-terminal boundary of the signal peptide may be identified pursuant to criteria routinely employed in the art for identifying that type of amino acid sequence element (e.g., Nielsen et al., Prot. Eng. 10:1-6 (1997) and von Heinje et al., Nucl. Acids. Res. 14:4683-4690 (1986)). Moreover, it is also recognized that, in some cases, cleavage of a signal sequence from a secreted polypeptide is not entirely uniform, resulting in more than one secreted species. These mature polypeptides, where the signal peptide is cleaved within no more than about 5 amino acids on either side of the C-terminal boundary of the signal peptide as identified herein, and the polynucleotides encoding them, are contemplated by the present invention.

“PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide variant” means a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, preferably an active PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, as defined herein having at least about 80% amino acid sequence identity with a full-length native sequence PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide sequence as disclosed herein, a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide sequence lacking the signal peptide as disclosed herein, an extracellular domain of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, with or without the signal peptide, as disclosed herein or any other fragment of a full-length PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide sequence as disclosed herein (such as those encoded by a nucleic acid that represents only a portion of the complete coding sequence for a full-length PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide). Such PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide variants include, for instance, PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides wherein one or more amino acid residues are added, or deleted, at the N- or C-terminus of the full-length native amino acid sequence. Ordinarily, a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide variant will have or will have at least about 80% amino acid sequence identity, alternatively will have or will have at least about 81%, 82%, 83%, 84%, 85%, 86%, 87%, 88%, 89%, 90%, 91%, 92%, 93%, 94%, 95%, 96%, 97%, 98%, or 99% amino acid sequence identity, to a full-length native sequence PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide sequence as disclosed herein, a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide sequence lacking the signal peptide as disclosed herein, an extracellular domain of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, with or without the signal peptide, as disclosed herein or any other specifically defined fragment of a full-length PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide sequence as disclosed herein. Ordinarily, PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 variant polypeptides are or are at least about 10 amino acids in length, alternatively are or are at least about 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600 amino acids in length, or more. Optionally, PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 variant polypeptides will have no more than one conservative amino acid substitution as compared to the native PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide sequence, alternatively will have or will have no more than 2, 3, 4, 5, 6, 7, 8, 9, or 10 conservative amino acid substitution as compared to the native PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide sequence.

“Percent (%) amino acid sequence identity” with respect to the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide sequences identified herein is defined as the percentage of amino acid residues in a candidate sequence that are identical with the amino acid residues in the specific PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, however, % amino acid sequence identity values are generated using the sequence comparison computer program ALIGN-2, wherein the complete source code for the ALIGN-2 program is provided in Table 1 below. The ALIGN-2 sequence comparison computer program was authored by Genentech, Inc. and the source code shown in Table 1 below has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or may be compiled from the source code provided in Table 1 below. The ALIGN-2 program should be compiled for use on a UNIX operating system, preferably digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. As examples of % amino acid sequence identity calculations using this method, Tables 2 and 3 demonstrate how to calculate the % amino acid sequence identity of the amino acid sequence designated “Comparison Protein” to the amino acid sequence designated “PRO”, wherein “PRO” represents the amino acid sequence of a hypothetical PRO polypeptide of interest, “Comparison Protein” represents the amino acid sequence of a polypeptide against which the “PRO” polypeptide of interest is being compared, and “X, “Y” and “Z” each represent different hypothetical amino acid residues. Unless specifically stated otherwise, all % amino acid sequence identity values used herein are obtained as described in the immediately preceding paragraph using the ALIGN-2 computer program.

“PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 variant polynucleotide” or “PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 variant nucleic acid sequence” means a nucleic acid molecule which encodes a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, preferably an active PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, as defined herein and which has at least about 80% nucleic acid sequence identity with a nucleotide acid sequence encoding a full-length native sequence PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide sequence as disclosed herein, a full-length native sequence PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide sequence lacking the signal peptide as disclosed herein, an extracellular domain of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, with or without the signal peptide, as disclosed herein or any other fragment of a full-length PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide sequence as disclosed herein (such as those encoded by a nucleic acid that represents only a portion of the complete coding sequence for a full-length PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide). Ordinarily, a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 variant polynucleotide will have or will have at least about 80% nucleic acid sequence identity, alternatively will have or will have at least about 81%, 82%, 83%, 84%, 85%, 86%, 87%, 88%, 89%, 90%, 91%, 92%, 93%, 94%, 95%, 96%, 97%, 98%, or 99% nucleic acid sequence identity with a nucleic acid sequence encoding a full-length native sequence PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide sequence as disclosed herein, a full-length native sequence PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide sequence lacking the signal peptide as disclosed herein, an extracellular domain of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, with or without the signal sequence, as disclosed herein or any other fragment of a full-length PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide sequence as disclosed herein. Variants do not encompass the native nucleotide sequence.

Ordinarily, PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 variant polynucleotides are or are at least about 5 nucleotides in length, alternatively are or are at least about 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides in length, wherein in this context the term “about” means the referenced nucleotide sequence length plus or minus 10% of that referenced length.

“Percent (%) nucleic acid sequence identity” with respect to PRO179-, PRO181-, PRO244-, PRO247-, PRO269-, PRO293-, PRO298-, PRO339-, PRO341-, PRO347-, PRO531-, PRO537-, PRO718-, PRO773-, PRO860-, PRO871-, PRO872-, PRO813-, PRO828-, PRO1100-, PRO1114-, PRO1115-, PRO1126-, PRO1133-, PRO1154-, PRO1185-, PRO1194-, PRO1287-, PRO1291-, PRO1293-, PRO1310-, PRO1312-, PRO1335-, PRO1339-, PRO2155-, PRO1356-, PRO1385-, PRO1412-, PRO1487-, PRO1758-, PRO1779-, PRO1785-, PRO1889-, PRO90318-, PRO3434-, PRO3579-, PRO4322-, PRO4343-, PRO4347-, PRO4403-, PRO4976-, PRO260-, PRO6014-, PRO6027-, PRO6181-, PRO6714-, PRO9922-, PRO7179-, PRO7476-, PRO9824-, PRO19814-, PRO19836-, PRO20088-, PRO70789-, PRO50298-, PRO51592-, PRO1757-, PRO4421-, PRO9903-, PRO1106-, PRO1411-, PRO1486-, PRO1565-, PRO4399- or PRO4404-encoding nucleic acid sequences identified herein is defined as the percentage of nucleotides in a candidate sequence that are identical with the nucleotides in the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 nucleic acid sequence of interest, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN or Megalign (DNASTAR) software. For purposes herein, however, % nucleic acid sequence identity values are generated using the sequence comparison computer program ALIGN-2, wherein the complete source code for the ALIGN-2 program is provided in Table 1 below. The ALIGN-2 sequence comparison computer program was authored by Genentech, Inc. and the source code shown in Table 1 below has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or may be compiled from the source code provided in Table 1 below. The ALIGN-2 program should be compiled for use on a UNIX operating system, preferably digital UNIX V4.0D. All sequence comparison parameters are set by the ALIGN-2 program and do not vary.

In situations where ALIGN-2 is employed for nucleic acid sequence comparisons, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows:

100 times the fraction W/Z

where W is the number of nucleotides scored as identical matches by the sequence alignment program ALIGN-2 in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C. As examples of % nucleic acid sequence identity calculations, Tables 4 and 5, demonstrate how to calculate the % nucleic acid sequence identity of the nucleic acid sequence designated “Comparison DNA” to the nucleic acid sequence designated “PRO-DNA”, wherein “PRO-DNA” represents a hypothetical PRO-encoding nucleic acid sequence of interest, “Comparison DNA” represents the nucleotide sequence of a nucleic acid molecule against which the “PRO-DNA” nucleic acid molecule of interest is being compared, and “N”, “L” and “V” each represent different hypothetical nucleotides. Unless specifically stated otherwise, all % nucleic acid sequence identity values used herein are obtained as described in the immediately preceding paragraph using the ALIGN-2 computer program.

The invention also provides PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 variant polynucleotides which are nucleic acid molecules that encode a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide and which are capable of hybridizing, preferably under stringent hybridization and wash conditions, to nucleotide sequences encoding a full-length PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide as disclosed herein. PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 variant polypeptides may be those that are encoded by a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 variant polynucleotide.

The term “full-length coding region” when used in reference to a nucleic acid encoding a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide refers to the sequence of nucleotides which encode the full-length PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide of the invention (which is often shown between start and stop codons, inclusive thereof, in the accompanying figures). The term “full-length coding region” when used in reference to an ATCC deposited nucleic acid refers to the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide-encoding portion of the cDNA that is inserted into the vector deposited with the ATCC (which is often shown between start and stop codons, inclusive thereof, in the accompanying figures).

“Isolated,” when used to describe the various polypeptides disclosed herein, means polypeptide that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. The invention provides that the polypeptide will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated polypeptide includes polypeptide in situ within recombinant cells, since at least one component of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide natural environment will not be present. Ordinarily, however, isolated polypeptide will be prepared by at least one purification step.

An “isolated” PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide-encoding nucleic acid or other polypeptide-encoding nucleic acid is a nucleic acid molecule that is identified and separated from at least one contaminant nucleic acid molecule with which it is ordinarily associated in the natural source of the polypeptide-encoding nucleic acid. An isolated polypeptide-encoding nucleic acid molecule is other than in the form or setting in which it is found in nature. Isolated polypeptide-encoding nucleic acid molecules therefore are distinguished from the specific polypeptide-encoding nucleic acid molecule as it exists in natural cells. However, an isolated polypeptide-encoding nucleic acid molecule includes polypeptide-encoding nucleic acid molecules contained in cells that ordinarily express the polypeptide where, for example, the nucleic acid molecule is in a chromosomal location different from that of natural cells.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

The term “epitope tagged” when used herein refers to a chimeric polypeptide comprising a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide fused to a “tag polypeptide”. The tag polypeptide has enough residues to provide an epitope against which an antibody can be made, yet is short enough such that it does not interfere with activity of the polypeptide to which it is fused. The tag polypeptide preferably also is fairly unique so that the antibody does not substantially cross-react with other epitopes. Suitable tag polypeptides generally have at least six amino acid residues and usually between about 8 and 50 amino acid residues (preferably, between about 10 and 20 amino acid residues).

“Active” or “activity” for the purposes herein refers to form(s) of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide which retain a biological and/or an immunological activity of native or naturally-occurring PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, wherein “biological” activity refers to a biological function (either inhibitory or stimulatory) caused by a native or naturally-occurring PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide other than the ability to induce the production of an antibody against an antigenic epitope possessed by a native or naturally-occurring PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide and an “immunological” activity refers to the ability to induce the production of an antibody against an antigenic epitope possessed by a native or naturally-occurring PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide.

The term “antagonist” is used in the broadest sense [unless otherwise qualified], and includes any molecule that partially or fully blocks, inhibits, or neutralizes a biological activity of a native PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide disclosed herein. In a similar manner, the term “agonist” is used in the broadest sense [unless otherwise qualified] and includes any molecule that mimics a biological activity of a native PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide disclosed herein. Suitable agonist or antagonist molecules specifically include agonist or antagonist antibodies or antibody fragments, fragments or amino acid sequence variants of native PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides, peptides, antisense oligonucleotides, small organic molecules, etc. Methods for identifying agonists or antagonists of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide may comprise contacting a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide with a candidate agonist or antagonist molecule and measuring a detectable change in one or more biological activities normally associated with the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide.

“Treating” or “treatment” or “alleviation” refers to both therapeutic treatment and prophylactic or preventative measures, wherein the object is to prevent or slow down (lessen) the targeted pathologic condition or disorder. A subject in need of treatment may already have the disorder, or may be prone to have the disorder or may be in whom the disorder is to be prevented.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

“Mammal” for purposes of treatment refers to any animal classified as a mammal, including humans, rodents such as rats or mice, domestic and farm animals, and zoo, sports, or pet animals, such as dogs, cats, cattle, horses, sheep, pigs, goats, rabbits, etc. Preferably, the mammal is human.

Administration “in combination with” one or more further therapeutic agents includes simultaneous (concurrent) and consecutive administration in any order.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

By “solid phase” is meant a non-aqueous matrix to which the antibody of the present invention can adhere. Examples of solid phases encompassed herein include those formed partially or entirely of glass (e.g., controlled pore glass), polysaccharides (e.g., agarose), polyacrylamides, polystyrene, polyvinyl alcohol and silicones. Depending on the context, the solid phase can comprise the well of an assay plate; in others it is a purification column (e.g., an affinity chromatography column). This term also includes a discontinuous solid phase of discrete particles, such as those described in U.S. Pat. No. 4,275,149.

A “liposome” is a small vesicle composed of various types of lipids, phospholipids and/or surfactant which is useful for delivery of a drug (such as a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide or antibody thereto) to a mammal. The components of the liposome are commonly arranged in a bilayer formation, similar to the lipid arrangement of biological membranes.

A “small molecule” is defined herein to have a molecular weight below about 500 Daltons.

An “effective amount” of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody, a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 binding oligopeptide, a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 binding organic molecule or an agonist or antagonist thereof as disclosed herein is an amount sufficient to carry out a specifically stated purpose. An “effective amount” may be determined empirically and in a routine manner, in relation to the stated purpose.

The term “therapeutically effective amount” refers to an amount of an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody, a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 binding oligopeptide, a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 binding organic molecule or other drug effective to “treat” a disease or disorder in a subject or mammal. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. See the definition herein of “treating”. To the extent the drug may prevent growth and/or kill existing cancer cells, it may be cytostatic and/or cytotoxic.

The phrases “cardiovascular, endothelial and angiogenic disorder”, “cardiovascular, endothelial and angiogenic dysfunction”, “cardiovascular, endothelial or angiogenic disorder” and “cardiovascular, endothelial or angiogenic dysfunction” are used interchangeably and refer in part to systemic disorders that affect vessels, such as diabetes mellitus, as well as diseases of the vessels themselves, such as of the arteries, capillaries, veins, and/or lymphatics. This would include indications that stimulate angiogenesis and/or cardiovascularization, and those that inhibit angiogenesis and/or cardiovascularization. Such disorders include, for example, arterial disease, such as atherosclerosis, hypertension, inflammatory vasculitides, Reynaud's disease and Reynaud's phenomenon, aneurysms, and arterial restenosis; venous and lymphatic disorders such as thrombophlebitis, lymphangitis, and lymphedema; and other vascular disorders such as peripheral vascular disease, cancer such as vascular tumors, e.g., hemangioma (capillary and cavernous), glomus tumors, telangiectasia, bacillary angiomatosis, hemangioendothelioma, angiosarcoma, haemangiopericytoma, Kaposi's sarcoma, lymphangioma, and lymphangiosarcoma, tumor angiogenesis, trauma such as wounds, burns, and other injured tissue, implant fixation, scarring, ischemia reperfusion injury, rheumatoid arthritis, cerebrovascular disease, renal diseases such as acute renal failure, or osteoporosis. This would also include angina, myocardial infarctions such as acute myocardial infarctions, cardiac hypertrophy, and heart failure such as CHF.

“Hypertrophy”, as used herein, is defined as an increase in mass of an organ or structure independent of natural growth that does not involve tumor formation. Hypertrophy of an organ or tissue is due either to an increase in the mass of the individual cells (true hypertrophy), or to an increase in the number of cells making up the tissue (hyperplasia), or both. Certain organs, such as the heart, lose the ability to divide shortly after birth. Accordingly, “cardiac hypertrophy” is defined as an increase in mass of the heart, which, in adults, is characterized by an increase in myocyte cell size and contractile protein content without concomitant cell division. The character of the stress responsible for inciting the hypertrophy, (e.g., increased preload, increased afterload, loss of myocytes, as in myocardial infarction, or primary depression of contractility), appears to play a critical role in determining the nature of the response. The early stage of cardiac hypertrophy is usually characterized morphologically by increases in the size of myofibrils and mitochondria, as well as by enlargement of mitochondria and nuclei. At this stage, while muscle cells are larger than normal, cellular organization is largely preserved. At a more advanced stage of cardiac hypertrophy, there are preferential increases in the size or number of specific organelles, such as mitochondria, and new contractile elements are added in localized areas of the cells, in an irregular manner. Cells subjected to long-standing hypertrophy show more obvious disruptions in cellular organization, including markedly enlarged nuclei with highly lobulated membranes, which displace adjacent myofibrils and cause breakdown of normal Z-band registration. The phrase “cardiac hypertrophy” is used to include all stages of the progression of this condition, characterized by various degrees of structural damage of the heart muscle, regardless of the underlying cardiac disorder. Hence, the term also includes physiological conditions instrumental in the development of cardiac hypertrophy, such as elevated blood pressure, aortic stenosis, or myocardial infarction.

“Heart failure” refers to an abnormality of cardiac function where the heart does not pump blood at the rate needed for the requirements of metabolizing tissues. The heart failure can be caused by a number of factors, including ischemic, congenital, rheumatic, or idiopathic forms.

“Congestive heart failure” (CHF) is a progressive pathologic state where the heart is increasingly unable to supply adequate cardiac output (the volume of blood pumped by the heart over time) to deliver the oxygenated blood to peripheral tissues. As CHF progresses, structural and hemodynamic damages occur. While these damages have a variety of manifestations, one characteristic symptom is ventricular hypertrophy. CHF is a common end result of a number of various cardiac disorders.

“Myocardial infarction” generally results from atherosclerosis of the coronary arteries, often with superimposed coronary thrombosis. It may be divided into two major types: transmural infarcts, in which myocardial necrosis involves the full thickness of the ventricular wall, and subendocardial (nontransmural) infarcts, in which the necrosis involves the subendocardium, the intramural myocardium, or both, without extending all the way through the ventricular wall to the epicardium. Myocardial infarction is known to cause both a change in hemodynamic effects and an alteration in structure in the damaged and healthy zones of the heart. Thus, for example, myocardial infarction reduces the maximum cardiac output and the stroke volume of the heart. Also associated with myocardial infarction is a stimulation of the DNA synthesis occurring in the interstice as well as an increase in the formation of collagen in the areas of the heart not affected.

As a result of the increased stress or strain placed on the heart in prolonged hypertension due, for example, to the increased total peripheral resistance, cardiac hypertrophy has long been associated with “hypertension”. A characteristic of the ventricle that becomes hypertrophic as a result of chronic pressure overload is an impaired diastolic performance. Fouad et al., J. Am. Coll. Cardiol., 4: 1500-1506 (1984); Smith et al., J. Am. Coll. Cardiol., 5: 869-874 (1985). A prolonged left ventricular relaxation has been detected in early essential hypertension, in spite of normal or supranormal systolic function. Hartford et al., Hypertension, 6: 329-338 (1984). However, there is no close parallelism between blood pressure levels and cardiac hypertrophy. Although improvement in left ventricular function in response to antihypertensive therapy has been reported in humans, patients variously treated with a diuretic (hydrochlorothiazide), a β-blocker (propranolol), or a calcium channel blocker (diltiazem), have shown reversal ofleft ventricular hypertrophy, without improvement in diastolic function. Inouye et al., Am. J. Cardiol., 53: 1583-7 (1984).

Another complex cardiac disease associated with cardiac hypertrophy is “hypertrophic cardiomyopathy”. This condition is characterized by a great diversity of morphologic, functional, and clinical features (Maron et al., N. Engl. J. Med., 316: 780-789 (1987); Spirito et al., N. Engl. J. Med., 320: 749-755 (1989); Louie and Edwards, Prog. Cardiovasc. Dis., 36: 275-308 (1994); Wigle et al., Circulation, 92: 1680-1692 (1995)), the heterogeneity of which is accentuated by the fact that it afflicts patients of all ages. Spirito et al., N. Engl. J. Med., 336: 775-785 (1997). The causative factors of hypertrophic cardiomyopathy are also diverse and little understood. In general, mutations in genes encoding sarcomeric proteins are associated with hypertrophic cardiomyopathy. Recent data suggest that β-myosin heavy chain mutations may account for approximately 30 to 40 percent of cases of familial hypertrophic cardiomyopathy. Watkins et al., N. Engl. J. Med., 326: 1108-1114 (1992); Schwartz et al, Circulation, 91: 532-540 (1995); Marian and Roberts, Circulation, 92: 1336-1347 (1995); Thierfelder et al., Cell, 77: 701-712 (1994); Watkins et al., Nat. Gen., 11: 434-437 (1995). Besides β-myosin heavy chain, other locations of genetic mutations include cardiac troponin T, alpha topomyosin, cardiac myosin binding protein C, essential myosin light chain, and regulatory myosin light chain. See, Malik and Watkins, Curr. Opin. Cardiol., 12: 295-302 (1997).

Supravalvular “aortic stenosis” is an inherited vascular disorder characterized by narrowing of the ascending aorta, but other arteries, including the pulmonary arteries, may also be affected. Untreated aortic stenosis may lead to increased intracardiac pressure resulting in myocardial hypertrophy and eventually heart failure and death. The pathogenesis of this disorder is not fully understood, but hypertrophy and possibly hyperplasia of medial smooth muscle are prominent features of this disorder. It has been reported that molecular variants of the elastin gene are involved in the development and pathogenesis of aortic stenosis. U.S. Pat. No. 5,650,282 issued Jul. 22, 1997.

“Valvular regurgitation” occurs as a result of heart diseases resulting in disorders of the cardiac valves. Various diseases, like rheumatic fever, can cause the shrinking or pulling apart of the valve orifice, while other diseases may result in endocarditis, an inflammation of the endocardium or lining membrane of the atrioventricular orifices and operation of the heart. Defects such as the narrowing of the valve stenosis or the defective closing of the valve result in an accumulation of blood in the heart cavity or regurgitation of blood past the valve. If uncorrected, prolonged valvular stenosis or insufficiency may result in cardiac hypertrophy and associated damage to the heart muscle, which may eventually necessitate valve replacement.

The term “immune related disease” means a disease in which a component of the immune system of a mammal causes, mediates or otherwise contributes to a morbidity in the mammal. Also included are diseases in which stimulation or intervention of the immune response has an ameliorative effect on progression of the disease. Included within this term are immune-mediated inflammatory diseases, non-immune-mediated inflammatory diseases, infectious diseases, immunodeficiency diseases, neoplasia, etc.

The term “T cell mediated disease” means a disease in which T cells directly or indirectly mediate or otherwise contribute to a morbidity in a mammal. The T cell mediated disease may be associated with cell mediated effects, lymphokine mediated effects, etc., and even effects associated with B cells if the B cells are stimulated, for example, by the lymphokines secreted by T cells.

Examples of immune-related and inflammatory diseases, some of which are immune or T cell mediated, include systemic lupus erythematosis, rheumatoid arthritis, juvenile chronic arthritis, spondyloarthropathies, systemic sclerosis (scleroderma), idiopathic inflammatory myopathies (dermatomyositis, polymyositis), Sjögren's syndrome, systemic vasculitis, sarcoidosis, autoimmune hemolytic anemia (immune pancytopenia, paroxysmal nocturnal hemoglobinuria), autoimmune thrombocytopenia (idiopathic thrombocytopenic purpura, immune-mediated thrombocytopenia), thyroiditis (Grave's disease, Hashimoto's thyroiditis, juvenile lymphocytic thyroiditis, atrophic thyroiditis), diabetes mellitus, immune-mediated renal disease (glomerulonephritis, tubulointerstitial nephritis), demyelinating diseases of the central and peripheral nervous systems such as multiple sclerosis, idiopathic demyelinating polyneuropathy or Guillain-Barré syndrome, and chronic inflammatory demyelinating polyneuropathy, hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis, inflammatory bowel disease (ulcerative colitis: Crohn's disease), gluten-sensitive enteropathy, and Whipple's disease, autoimmune or immune-mediated skin diseases including bullous skin diseases, erythema multiforme and contact dermatitis, psoriasis, allergic diseases such as asthma, allergic rhinitis, atopic dermatitis, food hypersensitivity and urticaria, immunologic diseases of the lung such as eosinophilic pneumonia, idiopathic pulmonary fibrosis and hypersensitivity pneumonitis, or transplantation associated diseases including graft rejection and graft-versus-host-disease. Infectious diseases including viral diseases such as AIDS (HIV infection), hepatitis A, B, C, D, and E, herpes, etc., bacterial infections, fungal infections, protozoal infections and parasitic infections.

An “autoimmune disease” herein is a disease or disorder arising from and directed against an individual's own tissues or organs or a co-segregate or manifestation thereof or resulting condition therefrom. In many of these autoimmune and inflammatory disorders, a number of clinical and laboratory markers may exist, including, but not limited to, hypergammaglobulinemia, high levels of autoantibodies, antigen-antibody complex deposits in tissues, benefit from corticosteroid or immunosuppressive treatments, and lymphoid cell aggregates in affected tissues. Without being limited to any one theory regarding B-cell mediated autoimmune disease, it is believed that B cells demonstrate a pathogenic effect in human autoimmune diseases through a multitude of mechanistic pathways, including autoantibody production, immune complex formation, dendritic and T-cell activation, cytokine synthesis, direct chemokine release, and providing a nidus for ectopic neo-lymphogenesis. Each of the se pathways may participate to different degrees in the pathology of autoimmune diseases.

“Autoimmune disease” can be an organ-specific disease (i.e., the immune response is specifically directed against an organ system such as the endocrine system, the hematopoietic system, the skin, the cardiopulmonary system, the gastrointestinal and liver systems, the renal system, the thyroid, the ears, the neuromuscular system, the central nervous system, etc.) or a systemic disease which can affect multiple organ systems (for example, systemic lupus erythematosus (SLE), rheumatoid arthritis, polymyositis, etc.). Preferred such diseases include autoimmune rheumatologic disorders (such as, for example, rheumatoid arthritis, Sjögren's syndrome, scleroderma, lupus such as SLE and lupus nephritis, polymyositis/dermatomyositis, cryoglobulinemia, anti-phospholipid antibody syndrome, and psoriatic arthritis), autoimmune gastrointestinal and liver disorders (such as, for example, inflammatory bowel diseases (e.g., ulcerative colitis and Crohn's disease), autoimmune gastritis and pernicious anemia, autoimmune hepatitis, primary biliary cirrhosis, primary sclerosing cholangitis, and celiac disease), vasculitis (such as, for example, ANCA-associated vasculitis, including Churg-Strauss vasculitis, Wegener's granulomatosis, and polyarteriitis), autoimmune neurological disorders (such as, for example, multiple sclerosis, opsoclonus myoclonus syndrome, myasthenia gravis, neuromyelitis optica, Parkinson's disease, Alzheimer's disease, and autoimmune polyneuropathies), renal disorders (such as, for example, glomerulonephritis, Goodpasture's syndrome, and Berger's disease), autoimmune dermatologic disorders (such as, for example, psoriasis, urticaria, hives, pemphigus vulgaris, bullous pemphigoid, and cutaneous lupus erythematosus), hematologic disorders (such as, for example, thrombocytopenic purpura, thrombotic thrombocytopenic purpura, post-transfusion purpura, and autoimmune hemolytic anemia), atherosclerosis, uveitis, autoimmune hearing diseases (such as, for example, inner ear disease and hearing loss), Behcet's disease, Raynaud's syndrome, organ transplant, and autoimmune endocrine disorders (such as, for example, diabetic-related autoimmune diseases such as insulin-dependent diabetes mellitus (IDDM), Addison's disease, and autoimmune thyroid disease (e.g., Graves' disease and thyroiditis)). More preferred such diseases include, for example, rheumatoid arthritis, ulcerative colitis, ANCA-associated vasculitis, lupus, multiple sclerosis, Sjögren's syndrome, Graves' disease, IDDM, pernicious anemia, thyroiditis, and glomerulonephritis.

Specific examples of other autoimmune diseases as defined herein, which in some cases encompass those listed above, include, but are not limited to, arthritis (acute and chronic, rheumatoid arthritis including juvenile-onset rheumatoid arthritis and stages such as rheumatoid synovitis, gout or gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, osteoarthritis, arthritis chronica progrediente, arthritis deformans, polyarthritis chronica primaria, reactive arthritis, menopausal arthritis, estrogen-depletion arthritis, and ankylosing spondylitis/rheumatoid spondylitis), autoimmune lymphoproliferative disease, inflammatory hyperproliferative skin diseases, psoriasis such as plaque psoriasis, gutatte psoriasis, pustular psoriasis, and psoriasis of the nails, atopy including atopic diseases such as hay fever and Job's syndrome, dermatitis including contact dermatitis, chronic contact dermatitis, exfoliative dermatitis, allergic dermatitis, allergic contact dermatitis, hives, dermatitis herpetiformis, nummular dermatitis, seborrheic dermatitis, non-specific dermatitis, primary irritant contact dermatitis, and atopic dermatitis, x-linked hyper IgM syndrome, allergic intraocular inflammatory diseases, urticaria such as chronic allergic urticaria and chronic idiopathic urticaria, including chronic autoimmune urticaria, myositis, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, ataxic sclerosis, neuromyelitis optica (NMO), inflammatory bowel disease (IBD) (for example, Crohn's disease, autoimmune-mediated gastrointestinal diseases, gastrointestinal inflammation, colitis such as ulcerative colitis, colitis ulcerosa, microscopic colitis, collagenous colitis, colitis polyposa, necrotizing enterocolitis, and transmural colitis, and autoimmune inflammatory bowel disease), bowel inflammation, pyoderma gangrenosum, erythema nodosum, primary sclerosing cholangitis, respiratory distress syndrome, including adult or acute respiratory distress syndrome (ARDS), meningitis, inflammation of all or part of the uvea, iritis, choroiditis, an autoimmune hematological disorder, graft-versus-host disease, angioedema such as hereditary angioedema, cranial nerve damage as in meningitis, herpes gestationis, pemphigoid gestationis, pruritis scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN (RPGN), proliferative nephritis, autoimmune polyglandular endocrine failure, balanitis including balanitis circumscripta plasmacellularis, balanoposthitis, erythema annulare centrifugum, erythema dyschromicum perstans, eythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, food allergies, drug allergies, insect allergies, rare allergic disorders such as mastocytosis, allergic reaction, eczema including allergic or atopic eczema, asteatotic eczema, dyshidrotic eczema, and vesicular palmoplantar eczema, asthma such as asthma bronchiale, bronchial asthma, and auto-immune asthma, conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus, including lupus nephritis, lupus cerebritis, pediatric lupus, non-renal lupus, extra-renal lupus, discoid lupus and discoid lupus erythematosus, alopecia lupus, SLE, such as cutaneous SLE or subacute cutaneous SLE, neonatal lupus syndrome (NLE), and lupus erythematosus disseminatus, juvenile onset (Type I) diabetes mellitus, including pediatric IDDM, adult onset diabetes mellitus (Type II diabetes), autoimmune diabetes, idiopathic diabetes insipidus, diabetic retinopathy, diabetic nephropathy, diabetic colitis, diabetic large-artery disorder, immune responses associated with acute and delayed hypersensitivity mediated by cytokines and T-lymphocytes, tuberculosis, sarcoidosis, granulomatosis including lymphomatoid granulomatosis, Wegener's granulomatosis, agranulocytosis, vasculitides, including vasculitis, large-vessel vasculitis (including polymyalgia rheumatica and giant-cell (Takayasu's) arteritis), medium-vessel vasculitis (including Kawasaki's disease and polyarteritis nodosa/periarteritis nodosa), microscopic polyarteritis, immunovasculitis, CNS vasculitis, cutaneous vasculitis, hypersensitivity vasculitis, necrotizing vasculitis such as systemic necrotizing vasculitis, and ANCA-associated vasculitis, such as Churg-Strauss vasculitis or syndrome (CSS) and ANCA-associated small-vessel vasculitis, temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia(s), cytopenias such as pancytopenia, leukopenia, diseases involving leukocyte diapedesis, CNS inflammatory disorders, Alzheimer's disease, Parkinson's disease, multiple organ injury syndrome such as those secondary to septicemia, trauma or hemorrhage, antigen-antibody complex-mediated diseases, anti-glomerular basement membrane disease, anti-phospholipid antibody syndrome, motoneuritis, allergic neuritis, Behcet's disease/syndrome, Castleman's syndrome, Goodpasture's syndrome, Reynaud's syndrome, Sjögren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, thermal injury due to an autoimmune condition, preeclampsia, an immune complex disorder such as immune complex nephritis, antibody-mediated nephritis, neuroinflammatory disorders, polyneuropathies, chronic neuropathy such as IgM polyneuropathies or IgM-mediated neuropathy, thrombocytopenia (as developed by myocardial infarction patients, for example), including thrombotic thrombocytopenic purpura (TTP), post-transfusion purpura (PTP), heparin-induced thrombocytopenia, and autoimmune or immune-mediated thrombocytopenia including, for example, idiopathic thrombocytopenic purpura (ITP) including chronic or acute ITP, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes such as autoimmune polyglandular syndromes, for example, type I (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, giant-cell hepatitis, chronic active hepatitis or autoimmune chronic active hepatitis, pneumonitis such as lymphoid interstitial pneumonitis (LIP), bronchiolitis obliterans (non-transplant) vs NSIP, Guillain-Barré syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome, Celiac or Coeliac disease, celiac sprue (gluten enteropathy), refractory sprue, idiopathic sprue, cryoglobulinemia such as mixed cryoglobulinemia, amylotrophic lateral sclerosis (ALS; Lou Gehrig's disease), coronary artery disease, autoimmune ear disease such as autoimmune inner ear disease (AIED), autoimmune hearing loss, polychondritis such as refractory or relapsed or relapsing polychondritis, pulmonary alveolar proteinosis, Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (e.g., benign monoclonal gammopathy and monoclonal gammopathy ofundetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal or segmental or focal segmental glomerulosclerosis (FSGS), endocrine ophthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, Dressler's syndrome, alopecia greata, alopecia totalis, CREST syndrome (calcinosis, Raynaud's phenomenon, esophageal dysmotility, sclerodactyl), and telangiectasia), male and female autoimmune infertility, e.g., due to anti-spermatozoan antibodies, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, parasitic diseases such as leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, fibrosing mediastinitis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis (acute or chronic), or Fuch's cyclitis, Henoch-Schonlein purpura, human immunodeficiency virus (HIV) infection, SCID, acquired immune deficiency syndrome (AIDS), echovirus infection, sepsis (systemic inflammatory response syndrome (SIRS)), endotoxemia, pancreatitis, thyroxicosis, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant-cell polymyalgia, chronic hypersensitivity pneumonitis, conjunctivitis, such as vernal catarrh, keratoconjunctivitis sicca, and epidemic keratoconjunctivitis, idiopathic nephritic syndrome, minimal change nephropathy, benign familial and ischemia-reperfusion injury, transplant organ reperfusion, retinal autoimmunity, joint inflammation, bronchitis, chronic obstructive airway/pulmonary disease, silicosis, aphthae, aphthous stomatitis, arteriosclerotic disorders (cerebral vascular insufficiency) such as arteriosclerotic encephalopathy and arteriosclerotic retinopathy, aspermiogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired spenic atrophy, non-malignant thymoma, lymphofollicular thymitis, vitiligo, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, autoimmune polyglandular syndromes, including polyglandular syndrome type I, adult-onset idiopathic hypoparathyroidism (AOIH), cardiomyopathy such as dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, allergic sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, spondyloarthropathies, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia syndrome, angiectasis, autoimmune disorders associated with collagen disease, rheumatism such as chronic arthrorheumatism, lymphadenitis, reduction in blood pressure response, vascular dysfunction, tissue injury, cardiovascular ischemia, hyperalgesia, renal ischemia, cerebral ischemia, and disease accompanying vascularization, allergic hypersensitivity disorders, glomerulonephritides, reperfusion injury, ischemic re-perfusion disorder, reperfusion injury of myocardial or other tissues, lymphomatous tracheobronchitis, inflammatory dermatoses, dermatoses with acute inflammatory components, multiple organ failure, bullous diseases, renal cortical necrosis, acute purulent meningitis or other central nervous system inflammatory disorders, ocular and orbital inflammatory disorders, granulocyte transfusion-associated syndromes, cytokine-induced toxicity, narcolepsy, acute serious inflammation, chronic intractable inflammation, pyelitis, endarterial hyperplasia, peptic ulcer, valvulitis, and endometriosis.

The phrase “anxiety related disorders” refers to disorders of anxiety, mood, and substance abuse, including but not limited to: depression, generalized anxiety disorders, attention deficit disorder, sleep disorder, hyperactivity disorder, obsessive compulsive disorder, schizophrenia, cognitive disorders, hyperalgesia and sensory disorders. Such disorders include the mild to moderate anxiety, anxiety disorder due to a general medical condition, anxiety disorder not otherwise specified, generalized anxiety disorder, panic attack, panic disorder with agoraphobia, panic disorder without agoraphobia, posttraumatic stress disorder, social phobia, social anxiety, autism, specific phobia, substance-induced anxiety disorder, acute alcohol withdrawal, obsessive compulsive disorder, agoraphobia, monopolar disorders, bipolar disorder I or II, bipolar disorder not otherwise specified, cyclothymic disorder, depressive disorder, major depressive disorder, mood disorder, substance-induced mood disorder, enhancement of cognitive function, loss of cognitive function associated with but not limited to Alzheimer's disease, stroke, or traumatic injury to the brain, seizures resulting from disease or injury including but not limited to epilepsy, learning disorders/disabilities, cerebral palsy. In addition, anxiety disorders may apply to personality disorders including but not limited to the following types: paranoid, antisocial, avoidant behavior, borderline personality disorders, dependent, histronic, narcissistic, obsessive-compulsive, schizoid, and schizotypal.

The term “lipid metabolic disorder” refers to abnormal clinical chemistry levels of cholesterol and triglycerides, wherein elevated levels of these lipids is an indication for atherosclerosis. Additionally, abnormal serum lipid levels may be an indication of various cardiovascular diseases including hypertension, stroke, coronary artery diseases, diabetes and/or obesity.

The phrase “eye abnormality” refers to such potential disorders of the eye as they may be related to atherosclerosis or various ophthalmological abnormalities. Such disorders include but are not limited to the following: retinal dysplasia, various retinopathies, restenosis, retinal artery obstruction or occlusion; retinal degeneration causing secondary atrophy of the retinal vasculature, retinitis pigmentosa, macular dystrophies, Stargardt's disease, congenital stationary night blindness, choroideremia, gyrate atrophy, Leber's congenital amaurosis, retinoschisis disorders, Wagner's syndrome, Usher syndromes, Zellweger syndrome, Saldino-Mainzer syndrome, Senior-Loken syndrome, Bardet-Biedl syndrome, Alport's syndrome, Alstrom's syndrome, Cockayne's syndrome, dysplaisa spondyloepiphysaria congentia, Flynn-Aird syndrome, Friedreich ataxia, Hallgren syndrome, Marshall syndrome, Albers-Schnoberg disease, Refsum's disease, Kearns-Sayre syndrome, Waardenburg's syndrome, Alagile syndrome, myotonic dystrophy, olivopontocerebellar atrophy, Pierre-Marie dunsdrome, Stickler syndrome, carotinemeia, cystinosis, Wolfram syndrome, Bassen-Kornzweig syndrome, abetalipoproteinemia, incontinentia pigmenti, Batten's disease, mucopolysaccharidoses, homocystinuria, or mannosidosis. Cataracts are also considered an eye abnormality and are associated with such systemic diseases as: Human Down's syndrome, Hallerman-Streiff syndrome, Lowe syndrome, galactosemia, Marfan syndrome, Trismoy 13-15 condition, Alport syndrome, myotonic dystrophy, Fabry disease, hypothroidisms, or Conradi syndrome. Other ocular developmental anomalies include: Aniridia, anterior segment and dysgenesis syndrome. Cataracts may also occur as a result of an intraocular infection or inflammation (uveitis).

A “growth inhibitory amount” of an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody, PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 binding oligopeptide or PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 binding organic molecule is an amount capable of inhibiting the growth of a cell, especially tumor, e.g., cancer cell, either in vitro or in vivo. A “growth inhibitory amount” of an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody, PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 binding oligopeptide or PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 binding organic molecule for purposes of inhibiting neoplastic cell growth may be determined empirically and in a routine manner.

A “cytotoxic amount” of an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody, PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 binding oligopeptide or PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 binding organic molecule is an amount capable of causing the destruction of a cell, especially tumor, e.g., cancer cell, either in vitro or in vivo. A “cytotoxic amount” of an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody, PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 binding oligopeptide or PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 binding organic molecule for purposes of inhibiting neoplastic cell growth may be determined empirically and in a routine manner.

The term “antibody” is used in the broadest sense and specifically covers, for example, single anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody monoclonal antibodies (including agonist, antagonist, and neutralizing antibodies), anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody compositions with polyepitopic specificity, polyclonal antibodies, single chain anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibodies, and fragments of anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibodies (see below) as long as they exhibit the desired biological or immunological activity. The term “immunoglobulin” (Ig) is used interchangeable with antibody herein.

An “isolated antibody” is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. The invention provides that the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or non-reducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The basic 4-chain antibody unit is a heterotetrameric glycoprotein composed of two identical light (L) chains and two identical heavy (H) chains (an IgM antibody consists of 5 of the basic heterotetramer unit along with an additional polypeptide called J chain, and therefore contain 10 antigen binding sites, while secreted IgA antibodies can polymerize to form polyvalent assemblages comprising 2-5 of the basic 4-chain units along with J chain). In the case of IgGs, the 4-chain unit is generally about 150,000 daltons. Each L chain is linked to a H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain (C_{L}) at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71 and Chapter 6.

The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated α, δ, ε, γ, and μ, respectively. The γ and α classes are further divided into subclasses on the basis of relatively minor differences in C_{H} sequence and function, e.g., humans express the following subclasses: IgG1, IgG2, IgG3, IgG4, IgA1, and IgA2.

The term “variable” refers to the fact that certain segments of the variable domains differ extensively in sequence among antibodies. The V domain mediates antigen binding and define specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g. around about residues 24-34 (L1), 50-56 (L2) and 89-97 (L3) in the V_{L}, and around about 1-35 (H1), 50-65 (H2) and 95-102 (H3) in the V_{H}; Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the V_{L}, and 26-32 (H1), 53-55 (H2) and 96-101 (H3) in the V_{H}; Chothia and Lesk J. Mol. Biol. 196:901-917 (1987)).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present invention may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

The monoclonal antibodies herein include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, Ape etc), and human constant region sequences.

An “intact” antibody is one which comprises an antigen-binding site as well as a C, and at least heavy chain constant domains, C_{H}1, C_{H}2 and C_{H}3. The constant domains may be native sequence constant domains (e.g. human native sequence constant domains) or amino acid sequence variant thereof. Preferably, the intact antibody has one or more effector functions.

“Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (see U.S. Pat. No. 5,641,870, Example 2; Zapata et al., Protein Eng. 8(10): 1057-1062 [1995]); single-chain antibody molecules; and multispecific antibodies formed from antibody fragments.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having divalent antigen-binding activity and is still capable of cross-linking antigen. Fab′ fragments differ from Fab fragments by having additional few residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, which region is also the part recognized by Fc receptors (FcR) found on certain types of cells.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995, infra.

The term “diabodies” refers to small antibody fragments prepared by constructing sFv fragments (see preceding paragraph) with short linkers (about 5-10 residues) between the V_{H} and V_{L} domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the V_{H} and V_{L} domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993).

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

A “species-dependent antibody,” e.g., a mammalian anti-human IgE antibody, is an antibody which has a stronger binding affinity for an antigen from a first mammalian species than it has for a homologue of that antigen from a second mammalian species. Normally, the species-dependent antibody “bind specifically” to a human antigen (i.e., has a binding affinity (1(d) value of no more than about 1×10⁻⁷ M, preferably no more than about 1×10⁻⁸ and most preferably no more than about 1×10⁻⁹ M) but has a binding affinity for a homologue of the antigen from a second non-human mammalian species which is at least about 50 fold, or at least about 500 fold, or at least about 1000 fold, weaker than its binding affinity for the human antigen. The species-dependent antibody can be of any of the various types of antibodies as defined above, but preferably is a humanized or human antibody.

A “PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 binding oligopeptide” is an oligopeptide that binds, preferably specifically, to a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide as described herein. PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 binding oligopeptides may be chemically synthesized using known oligopeptide synthesis methodology or may be prepared and purified using recombinant technology. PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 binding oligopeptides usually are or are at least about 5 amino acids in length, alternatively are or are at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length or more, wherein such oligopeptides that are capable of binding, preferably specifically, to a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide as described herein. PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 binding oligopeptides may be identified without undue experimentation using well known techniques. In this regard, it is noted that techniques for screening oligopeptide libraries for oligopeptides that are capable of specifically binding to a polypeptide target are well known in the art (see, e.g., U.S. Pat. Nos. 5,556,762, 5,750,373, 4,708,871, 4,833,092, 5,223,409, 5,403,484, 5,571,689, 5,663,143; PCT Publication Nos. WO 84/03506 and WO84/03564; Geysen et al., Proc. Natl. Acad. Sci. U.S.A., 81:3998-4002 (1984); Geysen et al., Proc. Natl. Acad. Sci. U.S.A., 82:178-182 (1985); Geysen et al., in Synthetic Peptides as Antigens, 130-149 (1986); Geysen et al., J. Immunol. Meth., 102:259-274 (1987); Schoofs et al., J. Immunol., 140:611-616 (1988), Cwirla, S. E. et al. (1990) Proc. Natl. Acad. Sci. USA, 87:6378; Lowman, H. B. et al. (1991) Biochemistry, 30:10832; Clackson, T. et al. (1991) Nature, 352: 624; Marks, J. D. et al. (1991), J. Mol. Biol., 222:581; Kang, A. S. et al. (1991) Proc. Natl. Acad. Sci. USA, 88:8363, and Smith, G. P. (1991) Current Opin. Biotechnol., 2:668).

A “PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 binding organic molecule” is an organic molecule other than an oligopeptide or antibody as defined herein that binds, preferably specifically, to a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide as described herein. PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 binding organic molecules may be identified and chemically synthesized using known methodology (see, e.g., PCT Publication Nos. WO00/00823 and WO00/39585). PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 binding organic molecules are usually less than about 2000 daltons in size, alternatively less than about 1500, 750, 500, 250 or 200 daltons in size, wherein such organic molecules that are capable of binding, preferably specifically, to a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide as described herein may be identified without undue experimentation using well known techniques. In this regard, it is noted that techniques for screening organic molecule libraries for molecules that are capable of binding to a polypeptide target are well known in the art (see, e.g., PCT Publication Nos. WO00/00823 and WO00/39585).

An antibody, oligopeptide or other organic molecule “which binds” an antigen of interest, e.g. a tumor-associated polypeptide antigen target, is one that binds the antigen with sufficient affinity such that the antibody, oligopeptide or other organic molecule is preferably useful as a diagnostic and/or therapeutic agent in targeting a cell or tissue expressing the antigen, and does not significantly cross-react with other proteins. The extent of binding of the antibody, oligopeptide or other organic molecule to a “non-target” protein will be less than about 10% of the binding of the antibody, oligopeptide or other organic molecule to its particular target protein as determined by fluorescence activated cell sorting (FACS) analysis or radioimmunoprecipitation (RIA). With regard to the binding of an antibody, oligopeptide or other organic molecule to a target molecule, the term “specific binding” or “specifically binds to” or is “specific for” a particular polypeptide or an epitope on a particular polypeptide target means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a Kd for the target of at least about 10⁻⁴M, alternatively at least about 10⁻⁵M, alternatively at least about 10⁻⁶ M, alternatively at least about 10⁻⁷M, alternatively at least about 10⁻⁸ M, alternatively at least about 10⁻⁹ M, alternatively at least about 10⁻¹⁰ M, alternatively at least about 10⁻¹¹ M, alternatively at least about 10⁻¹² M, or greater. The term “specific binding” refers to binding where a molecule binds to a particular polypeptide or epitope on a particular polypeptide without substantially binding to any other polypeptide or polypeptide epitope.

An antibody, oligopeptide or other organic molecule that “inhibits the growth of tumor cells expressing a “PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404” or a “growth inhibitory” antibody, oligopeptide or other organic molecule is one which results in measurable growth inhibition of cancer cells expressing or overexpressing the appropriate PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide. The PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide may be a transmembrane polypeptide expressed on the surface of a cancer cell or may be a polypeptide that is produced and secreted by a cancer cell. Preferred growth inhibitory anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibodies, oligopeptides or organic molecules inhibit growth of PRO179-, PRO181-, PRO244-, PRO247-, PRO269-, PRO293-, PRO298-, PRO339-, PRO341-, PRO347-, PRO531-, PRO537-, PRO718-, PRO773-, PRO860-, PRO871-, PRO872-, PRO813-, PRO828-, PRO1100-, PRO1114-, PROMS-, PRO1126-, PRO1133-, PRO1154-, PRO1185-, PRO1194-, PRO1287-, PRO1291-, PRO1293-, PRO1310-, PRO1312-, PRO1335-, PRO1339-, PRO2155-, PRO1356-, PRO1385-, PRO1412-, PRO1487-, PRO1758-, PRO1779-, PRO1785-, PRO1889-, PRO90318-, PRO3434-, PRO3579-, PRO4322-, PRO4343-, PRO4347-, PRO4403-, PRO4976-, PRO260-, PRO6014-, PRO6027-, PRO6181-, PRO6714-, PRO9922-, PRO7179-, PRO7476-, PRO9824-, PRO19814-, PRO19836-, PRO20088-, PRO70789-, PRO50298-, PRO51592-, PRO1757-, PRO4421-, PRO9903-, PRO1106-, PRO1411-, PRO1486-, PRO1565-, PRO4399- or PRO4404-expressing tumor cells by or by greater than 20%, preferably from about 20% to about 50%, and even more preferably, by or by greater than 50% (e.g., from about 50% to about 100%) as compared to the appropriate control, the control typically being tumor cells not treated with the antibody, oligopeptide or other organic molecule being tested. Growth inhibition can be measured at an antibody concentration of about 0.1 to 30 μg/ml or about 0.5 nM to 200 nM in cell culture, where the growth inhibition is determined 1-10 days after exposure of the tumor cells to the antibody. Growth inhibition of tumor cells in vivo can be determined in various ways. The antibody is growth inhibitory in vivo if administration of the anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody at about 1 μg/kg to about 100 mg/kg body weight results in reduction in tumor size or tumor cell proliferation within about days to 3 months from the first administration of the antibody, preferably within about 5 to 30 days.

An antibody, oligopeptide or other organic molecule which “induces apoptosis” is one which induces programmed cell death as determined by binding of annexin V, fragmentation of DNA, cell shrinkage, dilation of endoplasmic reticulum, cell fragmentation, and/or formation of membrane vesicles (called apoptotic bodies). The cell is usually one which overexpresses a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide. Preferably the cell is a tumor cell, e.g., a prostate, breast, ovarian, stomach, endometrial, lung, kidney, colon, bladder cell. Various methods are available for evaluating the cellular events associated with apoptosis. For example, phosphatidyl serine (PS) translocation can be measured by annexin binding; DNA fragmentation can be evaluated through DNA laddering; and nuclear/chromatin condensation along with DNA fragmentation can be evaluated by any increase in hypodiploid cells. Preferably, the antibody, oligopeptide or other organic molecule which induces apoptosis is one which results in or in about 2 to 50 fold, preferably in or in about 5 to 50 fold, and most preferably in or in about 10 to 50 fold, induction of annexin binding relative to untreated cell in an annexin binding assay.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation.

“Antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Natl. Acad. Sci. U.S.A. 95:652-656 (1998).

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain Inhibiting receptor FcγRIIB contains an immunoreceptor tyrosine-based inhibition motif (ITIM) in its cytoplasmic domain. (see review M. in Daëron, Annu. Rev. Immunol. 15:203-234 (1997)). FcRs are reviewed in Ravetch and Kinet, Annu. Rev. Immunol. 9:457-492 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

“Human effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred. The effector cells may be isolated from a native source, e.g., from blood.

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g., as described in Gazzano-Santoro et al., J. Immunol. Methods 202:163 (1996), may be performed.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia. More particular examples of such cancers include squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD). Preferably, the cancer comprises a tumor that expresses an IGF receptor, more preferably breast cancer, lung cancer, colorectal cancer, or prostate cancer, and most preferably breast or prostate cancer.

A “chemotherapeutic agent” is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omega1I (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhône-Poulenc Rorer, Antony, France); chloranbucil; GEMZAR® gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above.

Also included in this definition are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON-toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

The terms “cell proliferative disorder” and “proliferative disorder” refer to disorders that are associated with some degree of abnormal cell proliferation. In one aspect of the invention, the cell proliferative disorder is cancer.

“Tumor”, as used herein, refers to all neoplastic cell growth and proliferation, whether malignant or benign, and all pre-cancerous and cancerous cells and tissues.

An antibody, oligopeptide or other organic molecule which “induces cell death” is one which causes a viable cell to become nonviable. The cell is one which expresses a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, preferably a cell that overexpresses a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide as compared to a normal cell of the same tissue type. The PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide may be a transmembrane polypeptide expressed on the surface of a cancer cell or may be a polypeptide that is produced and secreted by a cancer cell. Preferably, the cell is a cancer cell, e.g., a breast, ovarian, stomach, endometrial, salivary gland, lung, kidney, colon, thyroid, pancreatic or bladder cell. Cell death in vitro may be determined in the absence of complement and immune effector cells to distinguish cell death induced by antibody-dependent cell-mediated cytotoxicity (ADCC) or complement dependent cytotoxicity (CDC). Thus, the assay for cell death may be performed using heat inactivated serum (i.e., in the absence of complement) and in the absence of immune effector cells. To determine whether the antibody, oligopeptide or other organic molecule is able to induce cell death, loss of membrane integrity as evaluated by uptake of propidium iodide (PI), trypan blue (see Moore et al. Cytotechnology 17:1-11 (1995)) or 7AAD can be assessed relative to untreated cells. Preferred cell death-inducing antibodies, oligopeptides or other organic molecules are those which induce PI uptake in the PI uptake assay in BT474 cells.

As used herein, the term “immunoadhesion” designates antibody-like molecules which combine the binding specificity of a heterologous protein (an “adhesion”) with the effector functions of immunoglobulin constant domains. Structurally, the immunoadhesions comprise a fusion of an amino acid sequence with the desired binding specificity which is other than the antigen recognition and binding site of an antibody (i.e., is “heterologous”), and an immunoglobulin constant domain sequence. The adhesion part of an immunoadhesion molecule typically is a contiguous amino acid sequence comprising at least the binding site of a receptor or a ligand. The immunoglobulin constant domain sequence in the immunoadhesion may be obtained from any immunoglobulin, such as IgG-1, IgG-2, IgG-3, or IgG-4 subtypes, IgA (including IgA-1 and IgA-2), IgE, IgD or IgM.

The word “label” when used herein refers to a detectable compound or composition which is conjugated directly or indirectly to the antibody so as to generate a “labeled” antibody. The label may be detectable by itself (e.g. radioisotope labels or fluorescent labels) or, in the case of an enzymatic label, may catalyze chemical alteration of a substrate compound or composition which is detectable.

“Replication-preventing agent” is an agent wherein replication, function, and/or growth of the cells is inhibited or prevented, or cells are destroyed, no matter what the mechanism, such as by apoptosis, angiostasis, cytosis, tumoricide, mytosis inhibition, blocking cell cycle progression, arresting cell growth, binding to tumors, acting as cellular mediators, etc. Such agents include a chemotherapeutic agent, cytotoxic agent, cytokine, growth-inhibitory agent, or anti-hormonal agent, e.g., an anti-estrogen compound such as tamoxifen, an anti-progesterone such as onapristone (see, EP 616 812); or an anti-androgen such as flutamide, as well as aromidase inhibitors, or a hormonal agent such as an androgen.

The term “cytotoxic agent” as used herein refers to a substance that inhibits or prevents the function of cells and/or causes destruction of cells. The term is intended to include radioactive isotopes (e.g., At²¹¹, I¹³¹, I¹²⁵, Y⁹⁰, Re¹⁸⁶, Re¹⁸⁸, Sm¹⁵³, Bi²¹², Pt³² and radioactive isotopes of Lu), chemotherapeutic agents e.g. methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents disclosed below. Other cytotoxic agents are described below. A tumoricidal agent causes destruction of tumor cells.

Preferred cytotoxic agents herein for the specific tumor types to use in combination with the antagonists herein are as follows:

1. Prostate cancer: androgens, docetaxel, paclitaxel, estramustine, doxorubicin, mitoxantrone, antibodies to ErbB2 domain(s) such as 2C4 (WO 01/00245; hybridoma ATCC HB-12697), which binds to a region in the extracellular domain of ErbB2 (e.g., any one or more residues in the region from about residue 22 to about residue 584 of ErbB2, inclusive), AVASTIN™ anti-vascular endothelial growth factor (VEGF), TARCEVA™ OSI-774 (erlotinib) (Genenetech and OSI Pharmaceuticals), or other epidermal growth factor receptor tyrosine kinase inhibitors (EGFR TKI's).
2. Stomach cancer: 5-fluorouracil (5FU), XELODA™ capecitabine, methotrexate, etoposide, cisplatin/carboplatin, pacliitaxel, docetaxel, gemcitabine, doxorubicin, and CPT-11 (camptothcin-11; irinotecan, USA Brand Name: CAMPTOSAR®).
3. Pancreatic cancer: gemcitabine, 5FU, XELODA™ capecitabine, CPT-11, docetaxel, paclitaxel, cisplatin, carboplatin, TARCEVA™ erlotinib, and other EGFR TKI's.
4. Colorectal cancer: 5FU, XELODA™ capecitabine, CPT-11, oxaliplatin, AVASTIN™ anti-VEGF, TARCEVA™ erlotinib and other EGFR TKI's, and ERBITUX™ (formerly known as IMC-C225) human:murine-chimerized monoclonal antibody that binds to EGFR and blocks the ability of EGF to initiate receptor activation and signaling to the tumor.
5. Renal cancer: IL-2, interferon alpha, AVASTIN™ anti-VEGF, MEGACE™ (Megestrol acetate) progestin, vinblastine, TARCEVA™ erlotinib, and other EGFR TKI's.

A “growth inhibitory agent” when used herein refers to a compound or composition which inhibits growth of a cell, especially a PRO179-, PRO181-, PRO244-, PRO247-, PRO269-, PRO293-, PRO298-, PRO339-, PRO341-, PRO347-, PRO531-, PRO537-, PRO718-, PRO773-, PRO860-, PRO871-, PRO872-, PRO813-, PRO828-, PRO1100-, PRO1114-, PRO1115-, PRO1126-, PRO1133-, PRO1154-, PRO1185-, PRO1194-, PRO1287-, PRO1291-, PRO1293-, PRO1310-, PRO1312-, PRO1335-, PRO1339-, PRO2155-, PRO1356-, PRO1385-, PRO1412-, PRO1487-, PRO1758-, PRO1779-, PRO1785-, PRO1889-, PRO90318-, PRO3434-, PRO3579-, PRO4322-, PRO4343-, PRO4347-, PRO4403-, PRO4976-, PRO260-, PRO6014-, PRO6027-, PRO6181-, PRO6714-, PRO9922-, PRO7179-, PRO7476-, PRO9824-, PRO19814-, PRO19836-, PRO20088-, PRO70789-, PRO50298-, PRO51592-, PRO1757-, PRO4421-, PRO9903-, PRO1106-, PRO1411-, PRO1486-, PRO1565-, PRO4399- or PRO4404-expressing cancer cell, either in vitro or in vivo. Thus, the growth inhibitory agent may be one which significantly reduces the percentage of PRO179-, PRO181-, PRO244-, PRO247-, PRO269-, PRO293-, PRO298-, PRO339-, PRO341-, PRO347-, PRO531-, PRO537-, PRO718-, PRO773-, PRO860-, PRO871-, PRO872-, PRO813-, PRO828-, PRO1100-, PRO1114-, PRO1115-, PRO1126-, PRO1133-, PRO1154-, PRO1185-, PRO1194-, PRO1287-, PRO1291-, PRO1293-, PRO1310-, PRO1312-, PRO1335-, PRO1339-, PRO2155-, PRO1356-, PRO1385-, PRO1412-, PRO1487-, PRO1758-, PRO1779-, PRO1785-, PRO1889-, PRO90318-, PRO3434-, PRO3579-, PRO4322-, PRO4343-, PRO4347-, PRO4403-, PRO4976-, PRO260-, PRO6014-, PRO6027-, PRO6181-, PRO6714-, PRO9922-, PRO7179-, PRO7476-, PRO9824-, PRO19814-, PRO19836-, PRO20088-, PRO70789-, PRO50298-, PRO51592-, PRO1757-, PRO4421-, PRO9903-, PRO1106-, PRO1411-, PRO1486-, PRO1565-, PRO4399- or PRO4404-expressing cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (WB Saunders: Philadelphia, 1995), especially p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

“Doxorubicin” is an anthracycline antibiotic. The full chemical name of doxorubicin is (8S-cis)-10-[(3-amino-2,3,6-trideoxy-α-L-lyxo-hexapyranosyl)oxy]-7,8,9,10-tetrahydro-6,8,11-trihydroxy-8-(hydroxyacetyl)-1-methoxy-5,12-naphthacenedione

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1a, IL-2, IL-3, IL-4, IL-5, IL-6, IL-7, IL-8, IL-9, IL-11, IL-12; a tumor necrosis factor such as TNF-α or TNF-β; and other polypeptide factors including LIF and kit ligand (KL). As used herein, the term cytokine includes proteins from natural sources or from recombinant cell culture and biologically active equivalents of the native sequence cytokines.

The term “package insert” is used to refer to instructions customarily included in commercial packages of therapeutic products, that contain information about the indications, usage, dosage, administration, contraindications and/or warnings concerning the use of such therapeutic products.

The term “gene” refers to (a) a gene containing at least one of the DNA sequences disclosed herein; (b) any DNA sequence that encodes the amino acid sequence encoded by the DNA sequences disclosed herein and/or; (c) any DNA sequence that hybridizes to the complement of the coding sequences disclosed herein. Preferably, the term includes coding as well as noncoding regions, and preferably includes all sequences necessary for normal gene expression.

The term “gene targeting” refers to a type of homologous recombination that occurs when a fragment of genomic DNA is introduced into a mammalian cell and that fragment locates and recombines with endogenous homologous sequences. Gene targeting by homologous recombination employs recombinant DNA technologies to replace specific genomic sequences with exogenous DNA of particular design.

The term “homologous recombination” refers to the exchange of DNA fragments between two DNA molecules or chromatids at the site of homologous nucleotide sequences.

The term “target gene” (alternatively referred to as “target gene sequence” or “target DNA sequence”) refers to any nucleic acid molecule, polynucleotide, or gene to be modified by homologous recombination. The target sequence includes an intact gene, an exon or intron, a regulatory sequence or any region between genes. The target gene my comprise a portion of a particular gene or genetic locus in the individual's genomic DNA.

“Disruption” of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 gene occurs when a fragment of genomic DNA locates and recombines with an endogenous homologous sequence wherein the disruption is a deletion of the native gene or a portion thereof, or a mutation in the native gene or wherein the disruption is the functional inactivation of the native gene. Alternatively, sequence disruptions may be generated by nonspecific insertional inactivation using a gene trap vector (i.e. non-human transgenic animals containing and expressing a randomly inserted transgene; see for example U.S. Pat. No. 6,436,707 issued Aug. 20, 2002). These sequence disruptions or modifications may include insertions, missense, frameshift, deletion, or substitutions, or replacements of DNA sequence, or any combination thereof. Insertions include the insertion of entire genes, which may be of animal, plant, fungal, insect, prokaryotic, or viral origin. Disruption, for example, can alter the normal gene product by inhibiting its production partially or completely or by enhancing the normal gene product's activity. Preferably, the disruption is a null disruption, wherein there is no significant expression of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 gene.

The term “native expression” refers to the expression of the full-length polypeptide encoded by the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 gene, at expression levels present in the wild-type mouse. Thus, a disruption in which there is “no native expression” of the endogenous PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 gene refers to a partial or complete reduction of the expression of at least a portion of a polypeptide encoded by an endogenous PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 gene of a single cell, selected cells, or all of the cells of a mammal.

The term “knockout” refers to the disruption of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 gene wherein the disruption results in: the functional inactivation of the native gene; the deletion of the native gene or a portion thereof; or a mutation in the native gene.

The term “knock-in” refers to the replacement of the mouse ortholog (or other mouse gene) with a human cDNA encoding any of the specific human PRO179-, PRO181-, PRO244-, PRO247-, PRO269-, PRO293-, PRO298-, PRO339-, PRO341-, PRO347-, PRO531-, PRO537-, PRO718-, PRO773-, PRO860-, PRO871-, PRO872-, PRO813-, PRO828-, PRO1100-, PRO1114-, PRO1115-, PRO1126-, PRO1133-, PRO1154-, PRO1185-, PRO1194-, PRO1287-, PRO1291-, PRO1293-, PRO1310-, PRO1312-, PRO1335-, PRO1339-, PRO2155-, PRO1356-, PRO1385-, PRO1412-, PRO1487-, PRO1758-, PRO1779-, PRO1785-, PRO1889-, PRO90318-, PRO3434-, PRO3579-, PRO4322-, PRO4343-, PRO4347-, PRO4403-, PRO4976-, PRO260-, PRO6014-, PRO6027-, PRO6181-, PRO6714-, PRO9922-, PRO7179-, PRO7476-, PRO9824-, PRO19814-, PRO19836-, PRO20088-, PRO70789-, PRO50298-, PRO51592-, PRO1757-, PRO4421-, PRO9903-, PRO1106-, PRO1411-, PRO1486-, PRO1565-, PRO4399- or PRO4404-encoding genes or variants thereof (ie. the disruption results in a replacement of a native mouse gene with a native human gene).

The term “construct” or “targeting construct” refers to an artificially assembled DNA segment to be transferred into a target tissue, cell line or animal. Typically, the targeting construct will include a gene or a nucleic acid sequence of particular interest, a marker gene and appropriate control sequences. As provided herein, the targeting construct comprises a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 targeting construct. A “PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 targeting construct” includes a DNA sequence homologous to at least one portion of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 gene and is capable of producing a disruption in a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 gene in a host cell.

The term “transgenic cell” refers to a cell containing within its genome a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 gene that has been disrupted, modified, altered, or replaced completely or partially by the method of gene targeting.

The term “transgenic animal” refers to an animal that contains within its genome a specific gene that has been disrupted or otherwise modified or mutated by the methods described herein or methods otherwise well known in the art. Preferably the non-human transgenic animal is a mammal. More preferably, the mammal is a rodent such as a rat or mouse. In addition, a “transgenic animal” may be a heterozygous animal (i.e., one defective allele and one wild-type allele) or a homozygous animal (i.e., two defective alleles). An embryo is considered to fall within the definition of an animal. The provision of an animal includes the provision of an embryo or foetus in utero, whether by mating or otherwise, and whether or not the embryo goes to term.

As used herein, the terms “selective marker” and position selection marker” refer to a gene encoding a product that enables only the cells that carry the gene to survive and/or grow under certain conditions. For example, plant and animal cells that express the introduced neomycin resistance (Neo^{r}) gene are resistant to the compound G418. Cells that do not carry the Neo^{r} gene marker are killed by G418. Other positive selection markers are known to, or are within the purview of, those of ordinary skill in the art.

The term “modulates” or “modulation” as used herein refers to the decrease, inhibition, reduction, amelioration, increase or enhancement of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 gene function, expression, activity, or alternatively a phenotype associated with PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 gene.

The term “ameliorates” or “amelioration” as used herein refers to a decrease, reduction or elimination of a condition, disease, disorder, or phenotype, including an abnormality or symptom.

The term “abnormality” refers to any disease, disorder, condition, or phenotype in which PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 is implicated, including pathological conditions and behavioral observations.

TABLE 1
/*
*
* C-C increased from 12 to 15
* Z is average of EQ
* B is average of ND
* match with stop is _M; stop-stop = 0; J (joker) match = 0
*/

| | | |
|---|---|---|
| #define _M | −8 | /* value of a match with a stop */ |

| | |
|---|---|
| int | _day[26][26] = { |

/* A B C D E F G H I J K L M N O P Q R S T U V W X Y Z */

| | |
|---|---|
| /* A */ | { 2, 0,−2, 0, 0,−4, 1,−1,−1, 0,−1,−2,−1, 0,_M, 1, 0,−2, 1, 1, 0, 0,−6, 0,−3, 0}, |
| /* B */ | { 0, 3,−4, 3, 2,−5, 0, 1,−2, 0, 0,−3,−2, 2,_M,−1, 1, 0, 0, 0, 0,−2,−5, 0,−3, 1}, |
| /* C */ | {−2,−4,15,−5,−5,−4,−3,−3,−2, 0,−5,−6,−5,−4,_M,−3,−5,−4, 0,−2, 0,−2,−8, 0, 0,−5}, |
| /* D */ | { 0, 3,−5, 4, 3,−6, 1, 1,−2, 0, 0,−4,−3, 2,_M,−1, 2,−1, 0, 0, 0,−2,−7, 0,−4, 2}, |
| /* E */ | { 0, 2,−5, 3, 4,−5, 0, 1,−2, 0, 0,−3,−2, 1,_M,−1, 2,−1, 0, 0, 0,−2,−7, 0,−4, 3}, |
| /* F */ | {−4,−5,−4,−6,−5, 9,−5,−2, 1, 0,−5, 2, 0,−4,_M,−5,−5,−4,−3,−3, 0,−1, 0, 0, 7,−5}, |
| /* G */ | { 1, 0,−3, 1, 0,−5, 5,−2,−3, 0,−2,−4,−3, 0,_M,−1,−1,−3, 1, 0, 0,−1,−7, 0,−5, 0}, |
| /* H */ | {−1, 1,−3, 1, 1,−2,−2, 6,−2, 0, 0,−2,−2, 2,_M, 0, 3, 2,−1,−1, 0,−2,−3, 0, 0, 2}, |
| /* I */ | {−1,−2,−2,−2,−2, 1,−3,−2, 5, 0,−2, 2, 2,−2,_M,−2,−2,−2,−1, 0, 0, 4,−5, 0,−1,−2}, |
| /* J */ | { 0, 0, 0, 0, 0, 0, 0, 0, 0, 0, 0, 0, 0, 0,_M, 0, 0, 0, 0, 0, 0, 0, 0, 0, 0, 0}, |
| /* K */ | {−1, 0,−5, 0, 0,−5,−2, 0,−2, 0, 5,−3, 0, 1,_M,−1, 1, 3, 0, 0, 0,−2,−3, 0,−4, 0}, |
| /* L */ | {−2,−3,−6,−4,−3, 2,−4,−2, 2, 0,−3, 6, 4,−3,_M,−3,−2,−3,−3,−1, 0, 2,−2, 0,−1,−2}, |
| /* M */ | {−1,−2,−5,−3,−2, 0,−3,−2, 2, 0, 0, 4, 6,−2,_M,−2,−1, 0,−2,−1, 0, 2,−4, 0,−2,−1}, |
| /* N */ | { 0, 2,−4, 2, 1,−4, 0, 2,−2, 0, 1,−3,−2, 2,_M,−1, 1, 0, 1, 0, 0,−2,−4, 0,−2, 1}, |
| /* O */ | {_M,_M,_M,_M,_M,_M,_M,_M,_M,_M,_M,_M,_M,_M, 0,_M,_M,_M, |
| | _M,_M,_M,_M,_M,_M,_M,_M}, |
| /* P */ | { 1,−1,−3,−1,−1,−5,−1, 0,−2, 0,−1,−3,−2,−1,_M, 6, 0, 0, 1, 0, 0,−1,−6, 0,−5, 0}, |
| /* Q */ | { 0, 1,−5, 2, 2,−5,−1, 3,−2, 0, 1,−2,−1, 1,_M, 0, 4, 1,−1,−1, 0,−2,−5, 0,−4, 3}, |
| /* R */ | {−2, 0,−4,−1,−1,−4,−3, 2,−2, 0, 3,−3, 0, 0,_M, 0, 1, 6, 0,−1, 0,−2, 2, 0,−4, 0}, |
| /* S */ | { 1, 0, 0, 0, 0,−3, 1,−1,−1, 0, 0,−3,−2, 1,_M, 1,−1, 0, 2, 1, 0,−1,−2, 0,−3, 0}, |
| /* T */ | { 1, 0,−2, 0, 0,−3, 0,−1, 0, 0, 0,−1,−1, 0,_M, 0,−1,−1, 1, 3, 0, 0,−5, 0,−3, 0}, |
| /* U */ | { 0, 0, 0, 0, 0, 0, 0, 0, 0, 0, 0, 0, 0, 0,_M, 0, 0, 0, 0, 0, 0, 0, 0, 0, 0, 0}, |
| /* V */ | { 0,−2,−2,−2,−2,−1,−1,−2, 4, 0,−2, 2, 2,−2,_M,−1,−2,−2,−1, 0, 0, 4,−6, 0,−2,−2}, |
| /* W */ | {−6,−5,−8,−7,−7, 0,−7,−3,−5, 0,−3,−2,−4,−4,_M,−6,−5, 2,−2,−5, 0,−6,17, 0, 0,−6}, |
| /* X */ | { 0, 0, 0, 0, 0, 0, 0, 0, 0, 0, 0, 0, 0, 0,_M, 0, 0, 0, 0, 0, 0, 0, 0, 0, 0, 0}, |
| /* Y */ | {−3,−3, 0,−4,−4, 7,−5, 0,−1, 0,−4,−1,−2,−2,_M,−5,−4,−4,−3,−3, 0,−2, 0, 0,10,−4}, |
| /* Z */ | { 0, 1,−5, 2, 3,−5, 0, 2,−2, 0, 0,−2,−1, 1,_M, 0, 3, 0, 0, 0, 0,−2,−6, 0,−4, 4} |
| }; | |
| /* | |
| */ | |

#include <stdio.h>
#include <ctype.h>

| | | | |
|---|---|---|---|
| #define | MAXJMP | 16 | /* max jumps in a diag */ |
| #define | MAXGAP | 24 | /* don't continue to penalize gaps larger than this */ |
| #define | JMPS | 1024 | /* max jmps in an path */ |
| #define | MX | 4 | /* save if there's at least MX−1 bases since last jmp */ |
| #define | DMAT | 3 | /* value of matching bases */ |
| #define | DMIS | 0 | /* penalty for mismatched bases */ |
| #define | DINS0 | 8 | /* penalty for a gap */ |
| #define | DINS1 | 1 | /* penalty per base */ |
| #define | PINS0 | 8 | /* penalty for a gap */ |
| #define | PINS1 | 4 | /* penalty per residue */ |

struct jmp {

| | | |
|---|---|---|
| short | n[MAXJMP]; | /* size of jmp (neg for dely) */ |
| unsigned short | x[MAXJMP]; | /* base no. of jmp in seq x */ |

| | |
|---|---|
| }; | /* limits seq to 2\hat{ }16 −1 */ |

struct diag {

| | | |
|---|---|---|
| int | score; | /* score at last jmp */ |
| long | offset; | /* offset of prev block */ |
| short | ijmp; | /* current jmp index */ |
| struct jmp | jp; | /* list of jmps */ |

};
struct path {

| | | |
|---|---|---|
| int | spc; | /* number of leading spaces */ |

| | |
|---|---|
| short | n[JMPS]; /* size of jmp (gap) */ |
| int | x[JMPS]; /* loc of jmp (last elem before gap) */ |

};

| char | | *ofile; | /* output file name */ |
|---|---|---|---|
| char | | *namex[2]; | /* seq names: getseqs( ) */ |
| char | | *prog; | /* prog name for err msgs */ |
| char | | *seqx[2]; | /* seqs: getseqs( ) */ |
| int | | dmax; | /* best diag: nw( ) */ |
| int | | dmax( ); | /* final diag */ |
| int | | dna; | /* set if dna: main( ) */ |
| int | | endgaps; | /* set if penalizing end gaps */ |
| int | | gapx, gapy; | /* total gaps in seqs */ |
| int | | len0, len1; | /* seq lens */ |
| int | | ngapx, ngapy; | /* total size of gaps */ |
| int | | smax; | /* max score: nw( ) */ |
| int | | *xbm; | /* bitmap for matching */ |
| long | | offset; | /* current offset in jmp file */ |
| struct | diag | *dx; | /* holds diagonals */ |
| struct | path | pp[2]; | /* holds path for seqs */ |

| | |
|---|---|
| char | *calloc( ), *malloc( ), *index( ), *strcpy( ); |
| char | *getseq( ), *g_calloc( ); |

/* Needleman-Wunsch alignment program
*
* usage: progs file1 file2
* where file1 and file2 are two dna or two protein sequences.
* The sequences can be in upper- or lower-case an may contain ambiguity
* Any lines beginning with ‘;’, ‘>’ or ‘<’ are ignored
* Max file length is 65535 (limited by unsigned short x in the jmp struct)
* A sequence with ⅓ or more of its elements ACGTU is assumed to be DNA
* Output is in the file “align.out”
*
* The program may create a tmp file in /tmp to hold info about traceback.
* Original version developed under BSD 4.3 on a vax 8650
*/
#include “nw.h”
#include “day.h”

| | |
|---|---|
| static | _dbval[26] = { |
| | 1,14,2,13,0,0,4,11,0,0,12,0,3,15,0,0,0,5,6,8,8,7,9,0,10,0 |
| }; | |
| static | _pbval[26] = { |
| | 1, 2\|(1<<(‘D’−‘A’))\|(1<<(‘N’−‘A’)), 4, 8, 16, 32, 64, |
| | 128, 256, 0xFFFFFFF, 1<<10, 1<<11, 1<<12, 1<<13, 1<<14, |
| | 1<<15, 1<<16, 1<<17, 1<<18, 1<<19, 1<<20, 1<<21, 1<<22, |
| | 1<<23, 1<<24, 1<<25\|(1<<(‘E’−‘A’))\|(1<<(‘Q’−‘A’)) |
| }; | |

| | |
|---|---|
| main(ac, av) | main |

| | |
|---|---|
| int | ac; |
| char | *av[ ]; |

{

prog = av[0];
if (ac != 3) {

fprintf(stderr,“usage: %s file1 file2\n”, prog);
fprintf(stderr,“where file1 and file2 are two dna or two protein sequences.\n”);
fprintf(stderr,“The sequences can be in upper- or lower-case\n”);
fprintf(stderr,“Any lines beginning with ‘;’ or ‘<’ are ignored\n”);
fprintf(stderr,“Output is in the file \”align.out\“\n”);
exit(1);

| } | |
|---|---|
| namex[0] = av[1]; | |
| namex[1] = av[2]; | |
| seqx[0] = getseq(namex[0], &len0); | |
| seqx[1] = getseq(namex[1], &len1); | |
| xbm = (dna)? _dbval : _pbval; | |
| endgaps = 0; | /* 1 to penalize endgaps */ |
| ofile = “align.out”; | /* output file */ |

| | |
|---|---|
| nw( ); | /* fill in the matrix, get the possible jmps */ |
| readjmps( ); | /* get the actual jmps */ |
| print( ); | /* print stats, alignment */ |
| cleanup(0); | /* unlink any tmp files */} |

/* do the alignment, return best score: main( )
* dna: values in Fitch and Smith, PNAS, 80, 1382-1386, 1983
* pro: PAM 250 values
* When scores are equal, we prefer mismatches to any gap, prefer
* a new gap to extending an ongoing gap, and prefer a gap in seqx
* to a gap in seq y.
*/

| | |
|---|---|
| nw( ) | nw |
| { | |

| | | |
|---|---|---|
| char | *px, *py; | /* seqs and ptrs */ |
| int | *ndely, *dely; | /* keep track of dely */ |
| int | ndelx, delx; | /* keep track of delx */ |
| int | *tmp; | /* for swapping row( ), row1 */ |
| int | mis; | /* score for each type */ |
| int | ins0, ins1; | /* insertion penalties */ |
| register | id; | /* diagonal index */ |
| register | ij; | /* jmp index */ |
| register | *col0, *col1; | /* score for curr, last row */ |
| register | xx, yy; | /* index into seqs */ |

dx = (struct diag *)g_calloc(“to get diags”, len0+len1+1, sizeof(struct diag));
ndely = (int *)g_calloc(“to get ndely”, len1+1, sizeof(int));
dely = (int *)g_calloc(“to get dely”, len1+1, sizeof(int));
col0 = (int *)g_calloc(“to get col0”, len1+1, sizeof(int));
col1 = (int *)g_calloc(“to get col1”, len1+1, sizeof(int));
ins0 = (dna)? DINS0 : PINS0;
ins1 = (dna)? DINS1 : PINS1;
smax = −10000;
if (endgaps) {

for (col0[0] = dely[0] = −ins0, yy = 1; yy <= len1; yy++) {

col0[yy] = dely[yy] = col0[yy−1] − ins1;
ndely[yy] = yy;

| | |
|---|---|
| } | |
| col0[0] = 0; | /* Waterman Bull Math Biol 84 */ |

}
else

for (yy = 1; yy <= len1; yy++)

dely[yy] = −ins0;

/* fill in match matrix
*/
for (px = seqx[0], xx = 1; xx <= len0; px++, xx++) {

/* initialize first entry in col
*/
if (endgaps) {

if (xx == 1)

col1[0] = delx = −(ins0+ins1);

else

col1[0] = delx = col0[0] − ins1;

ndelx = xx;

}
else {

col1[0] = 0;
delx = −ins0;
ndelx = 0;

}

...nw

for (py = seqx[1], yy = 1; yy <= len1; py++, yy++) {

mis = col0[yy−1];
if (dna)

mis += (xbm[*px−‘A’]&xbm[*py−‘A’])? DMAT : DMIS;

else

mis += _day[*px−‘A’][*py−‘A’];

/* update penalty for del in x seq;
* favor new del over ongong del
* ignore MAXGAP if weighting endgaps
*/
if (endgaps || ndely[yy] < MAXGAP) {

if (col0[yy] − ins0 >= dely[yy]) {

dely[yy] = col0[yy] − (ins0+ins1);
ndely[yy] = 1;

} else {

dely[yy] −= ins1;
ndely[yy]++;

}

} else {

if (col0[yy] − (ins0+ins1) >= dely[yy]) {

dely[yy] = col0[yy] − (ins0+ins1);
ndely[yy] = 1;

} else

ndely[yy]++;

}
/* update penalty for del in y seq;
* favor new del over ongong del
*/
if (endgaps || ndelx < MAXGAP) {

if (col1[yy−1] − ins0 >= delx) {

delx = col1[yy−1] − (ins0+ins1);
ndelx = 1;

} else {

delx −= ins1;
ndelx++;

}

} else {

if (col1[yy−1] − (ins0+ins1) >= delx) {

delx = col1[yy−1] − (ins0+ins1);
ndelx = 1;

} else

ndelx++;

}
/* pick the maximum score; we're favoring
* mis over any del and delx over dely
*/

...nw

id = xx − yy + len1 − 1;
if (mis >= delx && mis >= dely[yy])

col1[yy] = mis;

else if (delx >= dely[yy]) {

col1[yy] = delx;
ij = dx[id].ijmp;
if (dx[id].jp.n[0] && (!dna || (ndelx >= MAXJMP
&& xx > dx[id].jp.x[ij]+MX) || mis > dx[id].score+DINS0)) {

dx[id].ijmp++;
if (++ij >= MAXJMP) {

writejmps(id);
ij = dx[id].ijmp = 0;
dx[id].offset = offset;
offset += sizeof(struct jmp) + sizeof(offset);

}

}
dx[id].jp.n[ij] = ndelx;
dx[id].jp.x[ij] = xx;
dx[id].score = delx;

}
else {

col1[yy] = dely[yy];
ij = dx[id].ijmp;

if (dx[id].jp.n[0] && (!dna || (ndely[yy] >= MAXJMP

&& xx > dx[id].jp.x[ij]+MX) || mis > dx[id].score+DINS0)) {

dx[id].ijmp++;
if (++ij >= MAXJMP) {

writejmps(id);
ij = dx[id].ijmp = 0;
dx[id].offset = offset;
offset += sizeof(struct jmp) + sizeof(offset);

}

}
dx[id].jp.n[ij] = −ndely[yy];
dx[id].jp.x[ij] = xx;
dx[id].score = dely[yy];

}
if (xx == len0 && yy < len1) {

/* last col
*/
if (endgaps)

col1[yy] −= ins0+ins1*(len1−yy);

if (col1[yy] > smax) {

smax = col1[yy];
dmax = id;

}

}

}
if (endgaps && xx < len0)

col1[yy−1] −= ins0+ins1*(len0−xx);

if (col1[yy−1] > smax) {

smax = col1[yy−1];
dmax = id;

| | |
|---|---|
| } | |
| tmp = col0; col0 = col1; col1 = tmp; | } |

| | |
|---|---|
| (void) free((char *)ndely); | |
| (void) free((char *)dely); | |
| (void) free((char *)col0); | |
| (void) free((char *)col1); | } |
| /* | |
| * | |

* print( ) -- only routine visible outside this module
*
* static:
* getmat( ) -- trace back best path, count matches: print( )
* pr_align( ) -- print alignment of described in array p[ ]: print( )
* dumpblock( ) -- dump a block of lines with numbers, stars: pr_align( )
* nums( ) -- put out a number line: dumpblock( )
* putline( ) -- put out a line (name, [num], seq, [num]): dumpblock( )
* stars( ) - -put a line of stars: dumpblock( )
* stripname( ) -- strip any path and prefix from a seqname
*/
#include “nw.h”

| | | |
|---|---|---|
| #define SPC | 3 | |
| #define P_LINE | 256 | /* maximum output line */ |
| #define P_SPC | 3 | /* space between name or num and seq */ |

| | | |
|---|---|---|
| extern | _day[26][26]; | |
| int | olen; | /* set output line length */ |
| FILE | *fx; | /* output file */ |

| | |
|---|---|
| print( ) | print |
| { | |

| | | |
|---|---|---|
| int | lx, ly, firstgap, lastgap; | /* overlap */ |

if ((fx = fopen(ofile, “w”)) == 0) {

fprintf(stderr,“%s: can't write %s\n”, prog, ofile);
cleanup(1);

| } | |
|---|---|
| fprintf(fx, “<first sequence: %s (length = %d)\n”, namex[0], len0); | |
| fprintf(fx, “<second sequence: %s (length = %d)\n”, namex[1], len1); | |
| olen = 60; | |
| lx = len0; | |
| ly = len1; | |
| firstgap = lastgap = 0; | |
| if (dmax < len1 − 1) { | /* leading gap in x */ |

pp[0].spc = firstgap = len1 − dmax − 1;
ly −= pp[0].spc;

| | |
|---|---|
| } | |
| else if (dmax > len1 − 1) { | /* leading gap in y */ |

pp[1].spc = firstgap = dmax − (len1 − 1);
lx −= pp[1].spc;

| | |
|---|---|
| } | |
| if (dmax0 < len0 − 1) { | /* trailing gap in x */ |

lastgap = len0 − dmax0 −1;
lx −= lastgap;

}
else if (dmax0 > len0 − 1) { /* trailing gap in y */

lastgap = dmax0 − (len0 − 1);
ly −= lastgap;

}
getmat(lx, ly, firstgap, lastgap);
pr_align( ); }

/*
* trace back the best path, count matches
*/
static

| | |
|---|---|
| getmat(lx, ly, firstgap, lastgap) | getmat |

| | | |
|---|---|---|
| int | lx, ly; | /* “core” (minus endgaps) */ |
| int | firstgap, lastgap; | /* leading trailing overlap */ |

{

| | |
|---|---|
| int | nm, i0, i1, siz0, siz1; |
| char | outx[32]; |
| double | pct; |
| register | n0, n1; |
| register char | *p0, *p1; |

/* get total matches, score
*/
i0 = i1 = siz0 = siz1 = 0;
p0 = seqx[0] + pp[1].spc;
p1 = seqx[1] + pp[0].spc;
n0 = pp[1].spc + 1;
n1 = pp[0].spc + 1;
nm = 0;
while ( *p0 && *p1 ) {

if (siz0) {

p1++;
n1++;
siz0−−;

}
else if (siz1) {

p0++;
n0++;
siz1−−;

}
else {

if (xbm[*p0−‘A’]&xbm[*p1−‘A’])

nm++;

if (n0++ == pp[0].x[i0])

siz0 = pp[0].n[i0++];

if (n1++ == pp[1].x[i1])

siz1 = pp[1].n[i1++];

p0++;
p1++;

}

}
/* pct homology:
* if penalizing endgaps, base is the shorter seq
* else, knock off overhangs and take shorter core
*/
if (endgaps)

lx = (len0 < len1)? len0 : len1;

else

lx = (lx < ly)? lx : ly;

pct = 100.*(double)nm/(double)lx;
fprintf(fx, “\n”);
fprintf(fx, “<%d match%s in an overlap of %d: %.2f percent similarity\n”,

nm, (nm == 1)? “” : “es”, lx, pct);

| | |
|---|---|
| fprintf(fx, “<gaps in first sequence: %d”, gapx); | ...getmat |

if (gapx) {

(void) sprintf(outx, “ (%d %s%s)”,

ngapx, (dna)? “base”:“residue”, (ngapx == 1)? “”:“s”);

fprintf(fx,“%s”, outx);

fprintf(fx, “, gaps in second sequence: %d”, gapy);
if (gapy) {

(void) sprintf(outx, “ (%d %s%s)”,

ngapy, (dna)? “base”:“residue”, (ngapy == 1)? “”:“s”);

fprintf(fx,“%s”, outx);

}
if (dna)

fprintf(fx,
“\n<score: %d (match = %d, mismatch = %d, gap penalty = %d + %d per base)\n”,
smax, DMAT, DMIS, DINS0, DINS1);

else

fprintf(fx,
“\n<score: %d (Dayhoff PAM 250 matrix, gap penalty = %d + %d per residue)\n”,
smax, PINS0, PINS1);

if (endgaps)

fprintf(fx,
“<endgaps penalized. left endgap: %d %s%s, right endgap: %d %s%s\n”,
firstgap, (dna)? “base” : “residue”, (firstgap == 1)? “” : “s”,
lastgap, (dna)? “base” : “residue”, (lastgap == 1)? “” : “s”);

else

fprintf(fx, “<endgaps not penalized\n”);

}

| | | |
|---|---|---|
| static | nm; | /* matches in core -- for checking */ |
| static | lmax; | /* lengths of stripped file names */ |
| static | ij[2]; | /* jmp index for a path */ |
| static | nc[2]; | /* number at start of current line */ |
| static | ni[2]; | /* current elem number -- for gapping */ |
| static | siz[2]; | |
| static char | *ps[2]; | /* ptr to current element */ |
| static char | *po[2]; | /* ptr to next output char slot */ |
| static char | out[2][P_LINE]; | /* output line */ |
| static char | star[P_LINE]; | /* set by stars( ) */ |

/*
* print alignment of described in struct path pp[ ]
*/
static

| | |
|---|---|
| pr_align( ) | pr_align |
| { | |

| | | |
|---|---|---|
| int | nn; | /* char count */ |
| int | more; | |
| register | I; | |

for (I = 0, lmax = 0; I < 2; I++) {

nn = stripname(namex[i]);
if (nn > lmax)

lmax = nn;

| | |
|---|---|
| nc[i] = 1; | |
| ni[i] = 1; | |
| siz[i] = ij[i] = 0; | |
| ps[i] = seqx[i]; | |
| po[i] = out[i]; | } |

for (nn = nm = 0, more = 1; more; ) { ...pr_align

for (I = more = 0; I < 2; I++) {

/*
* do we have more of this sequence?
*/
if (!*ps[i])

continue;

| | |
|---|---|
| more++; | |
| if (pp[i].spc) { | /* leading space */ |

*po[i]++ = ‘ ’;
pp[i].spc−−;

| | |
|---|---|
| } | |
| else if (siz[i]) { | /* in a gap */ |

*po[i]++ = ‘-’;
siz[i]−−;

| | |
|---|---|
| } | |
| else { | /* we're putting a seq element |
| | */ |

*po[i] = *ps[i];
if (islower(*ps[i]))

*ps[i] = toupper(*ps[i]);

po[i]++;
ps[i]++;
/*
* are we at next gap for this seq?
*/
if (ni[i] == pp[i].x[ij[i]]) {

/*
* we need to merge all gaps
* at this location
*/
siz[i] = pp[i].n[ij[i]++];
while (ni[i] == pp[i].x[ij[i]])

siz[i] += pp[i].n[ij[i]++];

}
ni[i]++;

}

}
if (++nn == olen || !more && nn) {

dumpblock( );
for (I = 0; I < 2; I++)

po[i] = out[i];

nn = 0;

}

}

}
/*
* dump a block of lines, including numbers, stars: pr_align( )
*/
static

| | |
|---|---|
| dumpblock( ) | dumpblock |
| { | |

register I;
for (I = 0; I < 2; I++)

*po[i]−− = ‘\0’;

...dumpblock

(void) putc(‘\n’, fx);
for (I = 0; I < 2; I++) {

if (*out[i] && (*out[i] != ‘ ’ || *(po[i]) != ‘ ’)) {

if (I == 0)

nums(I);

if (I == 0 && *out[1])

stars( );

putline(I);
if (I == 0 && *out[1])

fprintf(fx, star);

if (I == 1)

nums(I);

}

}

| | |
|---|---|
| } | |
| /* | |
| * put out a number line: dumpblock( ) | |
| */ | |
| static | |
| nums(ix) | nums |

| | | |
|---|---|---|
| int | ix; | /* index in out[ ] holding seq line */ |

{

| | |
|---|---|
| char | nline[P_LINE]; |
| register | I, j; |
| register char | *pn, *px, *py; |

for (pn = nline, I = 0; I < lmax+P_SPC; I++, pn++)

*pn = ‘ ’;

for (I = nc[ix], py = out[ix]; *py; py++, pn++) {

if (*py == ‘ ’ || *py == ‘-’)

*pn = ‘ ’;

else {

if (I%10 == 0 || (I == 1 && nc[ix] != 1)) {

j = (I < 0)? −I : I;
for (px = pn; j; j /= 10, px−−)

*px = j%10 + ‘0’;

if (I < 0)

*px = ‘-’;

}
else

*pn = ‘ ’;

I++;

}

}
*pn = ‘\0’;
nc[ix] = I;
for (pn = nline; *pn; pn++)

(void) putc(*pn, fx);

(void) putc(‘\n’, fx);

}
/*
* put out a line (name, [num], seq, [num]): dumpblock( )
*/
static

| | |
|---|---|
| putline(ix) | putline |

| | | |
|---|---|---|
| int | ix; | { |

...putline

| | |
|---|---|
| int | I; |
| register char | *px; |

for (px = namex[ix], I = 0; *px && *px != ‘:’; px++, I++)

(void) putc(*px, fx);

for (; I < lmax+P_SPC; I++)

(void) putc(‘ ’, fx);

/* these count from 1:
* ni[ ] is current element (from 1)
* nc[ ] is number at start of current line
*/
for (px = out[ix]; *px; px++)

(void) putc(*px&0x7F, fx);

(void) putc(‘\n’, fx);

}
/*
* put a line of stars (seqs always in out[0], out[1]): dumpblock( )
*/
static

| | |
|---|---|
| stars( ) | stars |
| { | |

| | |
|---|---|
| int | I; |
| register char | *p0, *p1, cx, *px; |

if (!*out[0] || (*out[0] == ‘ ’ && *(po[0]) == ‘ ’) ||
!*out[1] || (*out[1] == ‘ ’ && *(po[1]) == ‘ ’))
return;
px = star;
for (I = lmax+P_SPC; I; I−−)

*px++ = ‘ ’;

for (p0 = out[0], p1 = out[1]; *p0 && *p1; p0++, p1++) {

if (isalpha(*p0) && isalpha(*p1)) {

if (xbm[*p0−‘A’]&xbm[*p1−‘A’]) {

cx = ‘*’;
nm++;

}
else if (!dna &&_day[*p0−‘A’][*P1−‘A’] > 0)

cx = ‘.’;

else

cx = ‘ ’;

}
else

cx = ‘ ’;

*px++ = cx;

}
*px++ = ‘\n’;
*px = ‘\0’;

}
/*
* strip path or prefix from pn, return len: pr_align( )
*/
static

| | |
|---|---|
| stripname(pn) | stripname |

| | | |
|---|---|---|
| char | *pn; | /* file name (may be path) */ |

{

| | |
|---|---|
| register char | *px, *py; |
| py = 0; | |

for (px = pn; *px; px++)

if (*px == ‘/’)

py = px + 1;

if (py)

(void) strcpy(pn, py);

return(strlen(pn));

}
/*
* cleanup( ) -- cleanup any tmp file
* getseq( ) -- read in seq, set dna, len, maxlen
* g_calloc( ) -- calloc( ) with error checkin
* readjmps( ) -- get the good jmps, from tmp file if necessary
* writejmps( ) -- write a filled array of jmps to a tmp file: nw( )
*/
#include “nw.h”
#include <sys/file.h>

| | | |
|---|---|---|
| char | *jname = “/tmp/homgXXXXXX”; | /* tmp file for jmps */ |
| FILE | *fj; | |
| int | cleanup( ); | /* cleanup tmp file */ |
| long | lseek( ); | |

/*
* remove any tmp file if we blow
*/

| | |
|---|---|
| cleanup(I) | cleanup |

| | |
|---|---|
| int | I; |

{

if (fj)

(void) unlink(jname);

exit(I);

}
/*
* read, return ptr to seq, set dna, len, maxlen
* skip lines starting with ‘;’, ‘<’, or ‘>’
* seq in upper or lower case
*/

| | |
|---|---|
| char | * |

| | |
|---|---|
| getseq(file, len) | getseq |

| | | |
|---|---|---|
| char | *file; | /* file name */ |
| int | *len; | /* seq len */ |

{

| | |
|---|---|
| char | line[1024], *pseq; |
| register char | *px, *py; |
| int | natgc, tlen; |
| FILE | *fp; |

if ((fp = fopen(file,“r”)) == 0) {

fprintf(stderr,“%s: can't read %s\n”, prog, file);
exit(1);

}
tlen = natgc = 0;
while (fgets(line, 1024, fp)) {

if (*line == ‘;’ || *line == ‘<’ || *line == ‘>’)

continue;

for (px = line; *px != ‘\n’; px++)

if (isupper(*px) || islower(*px))

tlen++;

}
if ((pseq = malloc((unsigned)(tlen+6))) == 0) {

fprintf(stderr,“%s: malloc( ) failed to get %d bytes for %s\n”, prog, tlen+6, file);
exit(1);

}
pseq[0] = pseq[1] = pseq[2] = pseq[3] = ‘\0’;

...getseq

py = pseq + 4;
*len = tlen;
rewind(fp);
while (fgets(line, 1024, fp)) {

if (*line == ‘;’ || *line == ‘<’ || *line == ‘>’)

continue;

for (px = line; *px != ‘\n’; px++) {

if (isupper(*px))

*py++ = *px;

else if (islower(*px))

*py++ = toupper(*px);

if (index(“ATGCU”,*(py−1)))

natgc++;

}

| | |
|---|---|
| | } |
| | *py++ = ‘\0’; |
| | *py = ‘\0’; |
| | (void) fclose(fp); |
| | dna = natgc > (tlen/3); |
| | return(pseq+4); |
| } | |
| char | * |

| | |
|---|---|
| g_calloc(msg, nx, sz) | g_calloc |

| | | |
|---|---|---|
| char | *msg; | /* program, calling routine */ |
| int | nx, sz; | /* number and size of elements */ |

{

| | |
|---|---|
| char | *px, *calloc( ); |

if ((px = calloc((unsigned)nx, (unsigned)sz)) == 0) {

if (*msg) {

fprintf(stderr, “%s: g_calloc( ) failed %s (n=%d, sz=%d)\n”, prog, msg, nx, sz);
exit(1);

}

}
return(px);

}
/*
* get final jmps from dx[ ] or tmp file, set pp[ ], reset dmax: main( )
*/

| | |
|---|---|
| readjmps( ) | readjmps |

{

| | |
|---|---|
| int | fd = −1; |
| int | siz, i0, i1; |

| | |
|---|---|
| register | I, j, xx; |
| if (fj) { | |

(void) fclose(fj);
if ((fd = open(jname, O_RDONLY, 0)) < 0) {

fprintf(stderr, “%s: can't open( ) %s\n”, prog, jname);
cleanup(1);

}

}
for (I = i0 = i1 = 0, dmax0 = dmax, xx = len0; ; I++) {

while (1) {

for (j = dx[dmax].ijmp; j >= 0 && dx[dmax].jp.x[j] >= xx; j−−)

;

...readjmps

if (j < 0 && dx[dmax].offset && fj) {

| (void) lseek(fd, dx[dmax].offset, 0); | |
|---|---|
| (void) read(fd, (char *)&dx[dmax].jp, sizeof(struct jmp)); | |
| (void) read(fd, (char *)&dx[dmax].offset, sizeof(dx[dmax].offset)); | |
| dx[dmax].ijmp = MAXJMP−1; | } |
| else | |

break; }

if (I >= JMPS) {

fprintf(stderr, “%s: too many gaps in alignment\n”, prog);
cleanup(1);

}
if (j >= 0) {

| siz = dx[dmax].jp.n[j]; | |
|---|---|
| xx = dx[dmax].jp.x[j]; | |
| dmax += siz; | |
| if (siz < 0) { | /* gap in second seq */ |

| | |
|---|---|
| pp[1].n[i1] = −siz; | |
| xx += siz; | |
| /* id = xx − yy + len1 − 1 | */ |
| pp[1].x[i1] = xx − dmax + len1 − 1; | |
| gapy++; | |
| ngapy −= siz; | |

/* ignore MAXGAP when doing endgaps */

siz = (−siz < MAXGAP || endgaps)? −siz : MAXGAP;
i1++;

}
else if (siz > 0) { /* gap in first seq */

pp[0].n[i0] = siz;
pp[0].x[i0] = xx;
gapx++;
ngapx += siz;

/* ignore MAXGAP when doing endgaps */

siz = (siz < MAXGAP || endgaps)? siz : MAXGAP;
i0++;

}

}
else

break;

| | |
|---|---|
| } | |
| /* reverse the order of jmps | */ |

for (j = 0, i0−−; j < i0; j++, i0−−) {

I = pp[0].n[j]; pp[0].n[j] = pp[0].n[i0]; pp[0].n[i0] = I;
I = pp[0].x[j]; pp[0].x[j] = pp[0].x[i0]; pp[0].x[i0] = I;

}
for (j = 0, i1−−; j < i1; j++, i1−−) {

I = pp[1].n[j]; pp[1].n[j] = pp[1].n[i1]; pp[1].n[i1] = I;
I = pp[1].x[j]; pp[1].x[j] = pp[1].x[i1]; pp[1].x[i1] = I;

}
if (fd >= 0)

(void) close(fd);

if (fj) {

(void) unlink(jname);
fj = 0;
offset = 0;

| | |
|---|---|
| } | } |

/*
* write a filled jmp struct offset of the prev one (if any): nw( )
*/

| | |
|---|---|
| writejmps(ix) | writejmps |

| | |
|---|---|
| int | ix; |

{

| | |
|---|---|
| char | *mktemp( ); |
| if (!fj) { | |

if (mktemp(jname) < 0) {

fprintf(stderr, “%s: can't mktemp( ) %s\n”, prog, jname);
cleanup(1);

}
if ((fj = fopen(jname, “w”)) == 0) {

fprintf(stderr, “%s: can't write %s\n”, prog, jname);
exit(1);

}

}
(void) fwrite((char *)&dx[ix].jp, sizeof(struct jmp), 1, fj);
(void) fwrite((char *)&dx[ix].offset, sizeof(dx[ix].offset), 1, fj);

}

TABLE 2

| | | |
|---|---|---|
| PRO | XXXXXXXXXXXXXXX | (Length = 15 |
| | | amino acids) |
| Comparison | XXXXXYYYYYYY | (Length = 12 |
| Protein | | amino acids) |

% amino acid sequence identity = (the number of identically matching amino acid residues between the two polypeptide sequences as determined by ALIGN-2) divided by (the total number of amino acid residues of the PRO polypeptide) = 5 divided by 15 = 33.3%

TABLE 3

| | | |
|---|---|---|
| PRO | XXXXXXXXXX | (Length = 10 |
| | | amino acids) |
| Comparison | XXXXXYYYYYYZZYZ | (Length = 15 |
| Protein | | amino acids) |

% amino acid sequence identity = (the number of identically matching amino acid residues between the two polypeptide sequences as determined by ALIGN-2) divided by (the total number of amino acid residues of the PRO polypeptide) = 5 divided by 10 = 50%

TABLE 4

| | | |
|---|---|---|
| PRO-DNA | NNNNNNNNNNNNNN | (Length = 14 |
| | | nucleotides) |
| Comparison | NNNNNNLLLLLLLLLL | (Length = 16 |
| DNA | | nucleotides) |

% nucleic acid sequence identity = (the number of identically matching nucleotides between the two nucleic acid sequences as determined by ALIGN-2) divided by (the total number of nucleotides of the PRO-DNA nucleic acid sequence) = 6 divided by 14 = 42.9%

TABLE 5

| | | |
|---|---|---|
| PRO-DNA | NNNNNNNNNNNN | (Length = 12 |
| | | nucleotides) |
| Comparison | NNNNLLLVV | (Length = 9 |
| DNA | | nucleotides) |

% nucleic acid sequence identity = (the number of identically matching nucleotides between the two nucleic acid sequences as determined by ALIGN-2) divided by (the total number of nucleotides of the PRO-DNA nucleic acid sequence) = 4 divided by 12 = 33.3%

### II. Compositions and Methods of the Invention

A. Full-Length PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 Polypeptides

The present invention provides newly identified and isolated nucleotide sequences encoding polypeptides referred to in the present application as PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides. In particular, cDNAs encoding various PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides have been identified and isolated, as disclosed in further detail in the Examples below. It is noted that proteins produced in separate expression rounds may be given different PRO numbers but the UNQ number is unique for any given DNA and the encoded protein, and will not be changed. However, for sake of simplicity, in the present specification the protein encoded by the full length native nucleic acid molecules disclosed herein as well as all further native homologues and variants included in the foregoing definition of PRO, will be referred to as “PRO/number”, regardless of their origin or mode of preparation.

As disclosed in the Examples below, various cDNA clones have been deposited with the ATCC. The actual nucleotide sequences of those clones can readily be determined by the skilled artisan by sequencing of the deposited clone using routine methods in the art. The predicted amino acid sequence can be determined from the nucleotide sequence using routine skill. For the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides and encoding nucleic acids described herein, Applicants have identified what is believed to be the reading frame best identifiable with the sequence information available at the time.

B. PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 Polypeptide Variants

In addition to the full-length native sequence PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides described herein, it is contemplated that PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 variants can be prepared. PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 variants can be prepared by introducing appropriate nucleotide changes into the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 DNA, and/or by synthesis of the desired PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide. Those skilled in the art will appreciate that amino acid changes may alter post-translational processes of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, such as changing the number or position of glycosylation sites or altering the membrane anchoring characteristics.

Variations in the native full-length sequence PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide or in various domains of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide described herein, can be made, for example, using any of the techniques and guidelines for conservative and non-conservative mutations set forth, for instance, in U.S. Pat. No. 5,364,934. Variations may be a substitution, deletion or insertion of one or more codons encoding the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide that results in a change in the amino acid sequence of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide as compared with the native sequence PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide. Optionally the variation is by substitution of at least one amino acid with any other amino acid in one or more of the domains of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide. Guidance in determining which amino acid residue may be inserted, substituted or deleted without adversely affecting the desired activity may be found by comparing the sequence of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions or substitutions of amino acids in the sequence and testing the resulting variants for activity exhibited by the full-length or mature native sequence.

PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide fragments are provided herein. Such fragments may be truncated at the N-terminus or C-terminus, or may lack internal residues, for example, when compared with a full length native protein. Certain fragments lack amino acid residues that are not essential for a desired biological activity of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide.

PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 fragments may be prepared by any of a number of conventional techniques. Desired peptide fragments may be chemically synthesized. An alternative approach involves generating PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 fragments by enzymatic digestion, e.g., by treating the protein with an enzyme known to cleave proteins at sites defined by particular amino acid residues, or by digesting the DNA with suitable restriction enzymes and isolating the desired fragment. Yet another suitable technique involves isolating and amplifying a DNA fragment encoding a desired polypeptide fragment, by polymerase chain reaction (PCR). Oligonucleotides that define the desired termini of the DNA fragment are employed at the 5′ and 3′ primers in the PCR. Preferably, PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide fragments share at least one biological and/or immunological activity with the native PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide disclosed herein.

Conservative substitutions of interest are shown in Table 6 under the heading of preferred substitutions. If such substitutions result in a change in biological activity, then more substantial changes, denominated exemplary substitutions in Table 6, or as further described below in reference to amino acid classes, are preferably introduced and the products screened.

TABLE 6

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; | Leu |
| | Phe; Norleucine | |
| Leu (L) | Norleucine; Ile; Val; | Ile |
| | Met; Ala; Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; | Leu |
| | Ala; Norleucine | |

Substantial modifications in function or immunological identity of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties: Amino acids may be grouped according to similarities in the properties of their side chains (in A. L. Lehninger, in Biochemistry, second ed., pp. 73-75, Worth Publishers, New York (1975)):

(1) non-polar: Ala (A), Val (V), Leu (L), Ile (I), Pro (P), Phe (F), Trp (W), Met (M)
(2) uncharged polar: Gly (G), Ser (S), Thr (T), Cys (C), Tyr (Y), Asn (N), Gln (O)
(3) acidic: Asp (D), Glu (E)
(4) basic: Lys (K), Arg (R), His(H)
Alternatively, naturally occurring residues may be divided into groups based on common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class. Such substituted residues also may be introduced into the conservative substitution sites or, more preferably, into the remaining (non-conserved) sites.

The variations can be made using methods known in the art such as oligonucleotide-mediated (site-directed) mutagenesis, alanine scanning, and PCR mutagenesis. Site-directed mutagenesis [Carter et al., Nucl. Acids Res., 13:4331 (1986); Zoller et al., Nucl. Acids Res., 10:6487 (1987)], cassette mutagenesis [Wells et al., Gene, 34:315 (1985)], restriction selection mutagenesis [Wells et al., Philos. Trans. R. Soc. London SerA, 317:415 (1986)] or other known techniques can be performed on the cloned DNA to produce the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 variant DNA.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant [Cunningham and Wells, Science, 244: 1081-1085 (1989)]. Alanine is also typically preferred because it is the most common amino acid. Further, it is frequently found in both buried and exposed positions [Creighton, The Proteins, (W.H. Freeman & Co., N.Y.); Chothia, J. Mol. Biol., 150:1 (1976)]. If alanine substitution does not yield adequate amounts of variant, an isoteric amino acid can be used.

C. Modifications of PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 Polypeptides

Covalent modifications of PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides are included within the scope of this invention. One type of covalent modification includes reacting targeted amino acid residues of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide with an organic derivatizing agent that is capable of reacting with selected side chains or the N- or C-terminal residues of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide. Derivatization with bifunctional agents is useful, for instance, for crosslinking PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides to a water-insoluble support matrix or surface for use in the method for purifying anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibodies, and vice-versa. Commonly used crosslinking agents include, e.g., 1,1-bis(diazoacetyl)-2-phenylethane, glutaraldehyde, N-hydroxysuccinimide esters, for example, esters with 4-azido salicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains [T. E. Creighton, Proteins: Structure and Molecular Properties, W.H. Freeman & Co., San Francisco, pp. 79-86 (1983)], acetylation of the N-terminal amine, and amidation of any C-terminal carboxyl group.

Another type of covalent modification of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide included within the scope of this invention comprises altering the native glycosylation pattern of the polypeptide. “Altering the native glycosylation pattern” is intended for purposes herein to mean deleting one or more carbohydrate moieties found in native sequence PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides (either by removing the underlying glycosylation site or by deleting the glycosylation by chemical and/or enzymatic means), and/or adding one or more glycosylation sites that are not present in the native sequence PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide. In addition, the phrase includes qualitative changes in the glycosylation of the native proteins, involving a change in the nature and proportions of the various carbohydrate moieties present.

Addition of glycosylation sites to the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide may be accomplished by altering the amino acid sequence. The alteration may be made, for example, by the addition of, or substitution by, one or more serine or threonine residues to the native sequence PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 (for O-linked glycosylation sites). The PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 amino acid sequence may optionally be altered through changes at the DNA level, particularly by mutating the DNA encoding the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide at preselected bases such that codons are generated that will translate into the desired amino acids.

Another means of increasing the number of carbohydrate moieties on the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide is by chemical or enzymatic coupling of glycosides to the polypeptide. Such methods are described in the art, e.g., in WO 87/05330 published 11 Sep. 1987, and in Aplin and Wriston, CRC Crit. Rev. Biochem., pp. 259-306 (1981).

Removal of carbohydrate moieties present on the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

Another type of covalent modification of PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides comprises linking the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide to one of a variety of nonproteinaceous polymers, e.g., polyethylene glycol (PEG), polypropylene glycol, or polyoxyalkylenes, in the manner set forth in U.S. Pat. No. 4,640,835; 4,496,689; 4,301,144; 4,670,417; 4,791,192 or 4,179,337.

The PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides of the present invention may also be modified in a way to form a chimeric molecule comprising the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide fused to another, heterologous polypeptide or amino acid sequence.

Such a chimeric molecule comprises a fusion of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide with a tag polypeptide which provides an epitope to which an anti-tag antibody can selectively bind. The epitope tag is generally placed at the amino- or carboxyl-terminus of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide. The presence of such epitope-tagged forms of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide can be detected using an antibody against the tag polypeptide. Also, provision of the epitope tag enables the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide to be readily purified by affinity purification using an anti-tag antibody or another type of affinity matrix that binds to the epitope tag. Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; an α-tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

The chimeric molecule may comprise a fusion of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide with an immunoglobulin or a particular region of an immunoglobulin. For a bivalent form of the chimeric molecule (also referred to as an “immunoadhesin”), such a fusion could be to the Fc region of an IgG molecule. The Ig fusions preferably include the substitution of a soluble (transmembrane domain deleted or inactivated) form of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide in place of at least one variable region within an Ig molecule. In a particularly preferred aspect of the invention, the immunoglobulin fusion includes the hinge, CH2 and CH3, or the hinge, CH1, CH2 and CH3 regions of an IgG1 molecule. For the production of immunoglobulin fusions see also U.S. Pat. No. 5,428,130 issued Jun. 27, 1995.

D. Preparation of PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 Polypeptides

The description below relates primarily to production of PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides by culturing cells transformed or transfected with a vector containing PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 nucleic acid. It is, of course, contemplated that alternative methods, which are well known in the art, may be employed to prepare PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides. For instance, the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 sequence, or portions thereof, may be produced by direct peptide synthesis using solid-phase techniques [see, e.g., Stewart et al., Solid-Phase Peptide Synthesis, W.H. Freeman Co., San Francisco, Calif. (1969); Merrifield, J. Am. Chem. Soc., 85:2149-2154 (1963)]. In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide may be chemically synthesized separately and combined using chemical or enzymatic methods to produce the full-length PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide.

1. Isolation of DNA Encoding PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 Polypeptides

DNA encoding PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides may be obtained from a cDNA library prepared from tissue believed to possess the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 mRNA and to express it at a detectable level. Accordingly, human PRO179-, PRO181-, PRO244-, PRO247-, PRO269-, PRO293-, PRO298-, PRO339-, PRO341-, PRO347-, PRO531-, PRO537-, PRO718-, PRO773-, PRO860-, PRO871-, PRO872-, PRO813-, PRO828-, PRO1100-, PRO1114-, PRO1115-, PRO1126-, PRO1133-, PRO1154-, PRO1185-, PRO1194-, PRO1287-, PRO1291-, PRO1293-, PRO1310-, PRO1312-, PRO1335-, PRO1339-, PRO2155-, PRO1356-, PRO1385-, PRO1412-, PRO1487-, PRO1758-, PRO1779-, PRO1785-, PRO1889-, PRO90318-, PRO3434-, PRO3579-, PRO4322-, PRO4343-, PRO4347-, PRO4403-, PRO4976-, PRO260-, PRO6014-, PRO6027-, PRO6181-, PRO6714-, PRO9922-, PRO7179-, PRO7476-, PRO9824-, PRO19814-, PRO19836-, PRO20088-, PRO70789-, PRO50298-, PRO51592-, PRO1757-, PRO4421-, PRO9903-, PRO1106-, PRO1411-, PRO1486-, PRO1565-, PRO4399- or PRO4404-DNA can be conveniently obtained from a cDNA library prepared from human tissue, such as described in the Examples. The PRO179-, PRO181-, PRO244-, PRO247-, PRO269-, PRO293-, PRO298-, PRO339-, PRO341-, PRO347-, PRO531-, PRO537-, PRO718-, PRO773-, PRO860-, PRO871-, PRO872-, PRO813-, PRO828-, PRO1100-, PRO1114-, PRO1115-, PRO1126-, PRO1133-, PRO1154-, PRO1185-, PRO1194-, PRO1287-, PRO1291-, PRO1293-, PRO1310-, PRO1312-, PRO1335-, PRO1339-, PRO2155-, PRO1356-, PRO1385-, PRO1412-, PRO1487-, PRO1758-, PRO1779-, PRO1785-, PRO1889-, PRO90318-, PRO3434-, PRO3579-, PRO4322-, PRO4343-, PRO4347-, PRO4403-, PRO4976-, PRO260-, PRO6014-, PRO6027-, PRO6181-, PRO6714-, PRO9922-, PRO7179-, PRO7476-, PRO9824-, PRO19814-, PRO19836-, PRO20088-, PRO70789-, PRO50298-, PRO51592-, PRO1757-, PRO4421-, PRO9903-, PRO1106-, PRO1411-, PRO1486-, PRO1565-, PRO4399- or PRO4404-encoding gene may also be obtained from a genomic library or by known synthetic procedures (e.g., automated nucleic acid synthesis).

Libraries can be screened with probes (such as antibodies to the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide or oligonucleotides of at least about 20-80 bases) designed to identify the gene of interest or the protein encoded by it. Screening the cDNA or genomic library with the selected probe may be conducted using standard procedures, such as described in Sambrook et al., Molecular Cloning: A Laboratory Manual (New York: Cold Spring Harbor Laboratory Press, 1989). An alternative means to isolate the gene encoding PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 is to use PCR methodology [Sambrook et al., supra; Dieffenbach et al., PCR Primer: A Laboratory Manual (Cold Spring Harbor Laboratory Press, 1995)]. The Examples below describe techniques for screening a cDNA library. The oligonucleotide sequences selected as probes should be of sufficient length and sufficiently unambiguous that false positives are minimized. The oligonucleotide is preferably labeled such that it can be detected upon hybridization to DNA in the library being screened. Methods of labeling are well known in the art, and include the use of radiolabels like ³²P-labeled ATP, biotinylation or enzyme labeling. Hybridization conditions, including moderate stringency and high stringency, are provided in Sambrook et al., supra.

Sequences identified in such library screening methods can be compared and aligned to other known sequences deposited and available in public databases such as GenBank or other private sequence databases. Sequence identity (at either the amino acid or nucleotide level) within defined regions of the molecule or across the full-length sequence can be determined using methods known in the art and as described herein.

Nucleic acid having protein coding sequence may be obtained by screening selected cDNA or genomic libraries using the deduced amino acid sequence disclosed herein for the first time, and, if necessary, using conventional primer extension procedures as described in Sambrook et al., supra, to detect precursors and processing intermediates of mRNA that may not have been reverse-transcribed into cDNA.

2. Selection and Transformation of Host Cells

Host cells are transfected or transformed with expression or cloning vectors described herein for PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: a Practical Approach, M. Butler, ed. (IRL Press, 1991) and Sambrook et al., supra.

Methods of eukaryotic cell transfection and prokaryotic cell transformation are known to the ordinarily skilled artisan, for example, CaCl₂, CaPO₄, liposome-mediated and electroporation. Depending on the host cell used, transformation is performed using standard techniques appropriate to such cells. The calcium treatment employing calcium chloride, as described in Sambrook et al., supra, or electroporation is generally used for prokaryotes. Infection with Agrobacterium tumefaciens is used for transformation of certain plant cells, as described by Shaw et al., Gene, 23:315 (1983) and WO 89/05859 published 29 Jun. 1989. For mammalian cells without such cell walls, the calcium phosphate precipitation method of Graham and van der Eb, Virology, 52:456-457 (1978) can be employed. General aspects of mammalian cell host system transfections have been described in U.S. Pat. No. 4,399,216. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see Keown et al., Methods in Enzymology, 185:527-537 (1990) and Mansour et al., Nature, 336:348-352 (1988).

Suitable host cells for cloning or expressing the DNA in the vectors herein include prokaryote, yeast, or higher eukaryote cells. Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5 772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. These examples are illustrative rather than limiting. Strain W3110 is one particularly preferred host or parent host because it is a common host strain for recombinant DNA product fermentations. Preferably, the host cell secretes minimal amounts of proteolytic enzymes. For example, strain W3110 may be modified to effect a genetic mutation in the genes encoding proteins endogenous to the host, with examples of such hosts including E. coli W3110 strain 1A2, which has the complete genotype tonA; E. coli W3110 strain 9E4, which has the complete genotype tonA ptr3; E. coli W3110 strain 27C7 (ATCC 55,244), which has the complete genotype tonA ptr3 phoA E15 (argF-lac)169 degP ompT kan^{r}; E. coli W3110 strain 37D6, which has the complete genotype tonA ptr3 phoA E15 (argF-lac)169 degP ompT rbs7 ilvG kan^{r}; E. coli W3110 strain 40B4, which is strain 37D6 with a non-kanamycin resistant degP deletion mutation; and an E. coli strain having mutant periplasmic protease disclosed in U.S. Pat. No. 4,946,783 issued 7 Aug. 1990. Alternatively, in vitro methods of cloning, e.g., PCR or other nucleic acid polymerase reactions, are suitable.

In addition to prokaryotes, eukaryotic microbes such as filamentous fungi or yeast are suitable cloning or expression hosts for PRO179-, PRO181-, PRO244-, PRO247-, PRO269-, PRO293-, PRO298-, PRO339-, PRO341-, PRO347-, PRO531-, PRO537-, PRO718-, PRO773-, PRO860-, PRO871-, PRO872-, PRO813-, PRO828-, PRO1100-, PRO1114-, PRO1115-, PRO1126-, PRO1133-, PRO1154-, PRO1185-, PRO1194-, PRO1287-, PRO1291-, PRO1293-, PRO1310-, PRO1312-, PRO1335-, PRO1339-, PRO2155-, PRO1356-, PRO1385-, PRO1412-, PRO1487-, PRO1758-, PRO1779-, PRO1785-, PRO1889-, PRO90318-, PRO3434-, PRO3579-, PRO4322-, PRO4343-, PRO4347-, PRO4403-, PRO4976-, PRO260-, PRO6014-, PRO6027-, PRO6181-, PRO6714-, PRO9922-, PRO7179-, PRO7476-, PRO9824-, PRO19814-, PRO19836-, PRO20088-, PRO70789-, PRO50298-, PRO51592-, PRO1757-, PRO4421-, PRO9903-, PRO1106-, PRO1411-, PRO1486-, PRO1565-, PRO4399- or PRO4404-encoding vectors. Saccharomyces cerevisiae is a commonly used lower eukaryotic host microorganism. Others include Schizosaccharomyces pombe (Beach and Nurse, Nature, 290: 140 [1981]; EP 139,383 published 2 May 1985); Kluyveromyces hosts (U.S. Pat. No. 4,943,529; Fleer et al., Bio/Technology, 9:968-975 (1991)) such as, e.g., K. lactis (MW98-8C, CBS683, CBS4574; Louvencourt et al., J. Bacteriol., 154(2):737-742 [1983]), K. fragilis (ATCC 12,424), K. bulgaricus (ATCC 16,045), K. wickeramii (ATCC 24,178), K. waltii (ATCC 56,500), K. drosophilarum (ATCC 36,906; Van den Berg et al., Bio/Technology, 8:135 (1990)), K. thermotolerans, and K. marxianus; yarrowia (EP 402,226); Pichia pastoris (EP 183,070; Sreekrishna et al., J. Basic Microbiol., 28:265-278 [1988]); Candida; Trichoderma reesia (EP 244,234); Neurospora crassa (Case et al., Proc. Natl. Acad. Sci. USA, 76:5259-5263 [1979]); Schwanniomyces such as Schwanniomyces occidentalis (EP 394,538 published 31 Oct. 1990); and filamentous fungi such as, e.g., Neurospora, Penicillium, Tolypocladium (WO 91/00357 published 10 Jan. 1991), and Aspergillus hosts such as A. nidulans (Ballance et al., Biochem. Biophys. Res. Commun., 112:284-289 [1983]; Tilburn et al., Gene, 26:205-221 [1983]; Yelton et al., Proc. Natl. Acad. Sci. USA, 81: 1470-1474 [1984]) and A. niger (Kelly and Hynes, EMBO J., 4:475-479 [1985]). Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts may be found in C. Anthony, The Biochemistry of Methylotrophs, 269 (1982).

Suitable host cells for the expression of glycosylated PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides are derived from multicellular organisms. Examples of invertebrate cells include insect cells such as Drosophila S2 and Spodoptera S19, as well as plant cells. Examples of useful mammalian host cell lines include Chinese hamster ovary (CHO) and COS cells. More specific examples include monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); Chinese hamster ovary cells/−DHFR (CHO, Urlaub and Chasin, Proc. Natl. Acad. Sci. USA, 77:4216 (1980)); mouse sertoli cells (TM4, Mather, Biol. Reprod., 23:243-251 (1980)); human lung cells (W138, ATCC CCL 75); human liver cells (Hep G2, HB 8065); and mouse mammary tumor (MMT 060562, ATCC CCL51). The selection of the appropriate host cell is deemed to be within the skill in the art.

3. Selection and Use of a Replicable Vector

The nucleic acid (e.g., cDNA or genomic DNA) encoding PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides may be inserted into a replicable vector for cloning (amplification of the DNA) or for expression. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the PRO179-, PRO181-, PRO244-, PRO247-, PRO269-, PRO293-, PRO298-, PRO339-, PRO341-, PRO347-, PRO531-, PRO537-, PRO718-, PRO773-, PRO860-, PRO871-, PRO872-, PRO813-, PRO828-, PRO1100-, PRO1114-, PRO1115-, PRO1126-, PRO1133-, PRO1154-, PRO1185-, PRO1194-, PRO1287-, PRO1291-, PRO1293-, PRO1310-, PRO1312-, PRO1335-, PRO1339-, PRO2155-, PRO1356-, PRO1385-, PRO1412-, PRO1487-, PRO1758-, PRO1779-, PRO1785-, PRO1889-, PRO90318-, PRO3434-, PRO3579-, PRO4322-, PRO4343-, PRO4347-, PRO4403-, PRO4976-, PRO260-, PRO6014-, PRO6027-, PRO6181-, PRO6714-, PRO9922-, PRO7179-, PRO7476-, PRO9824-, PRO19814-, PRO19836-, PRO20088-, PRO70789-, PRO50298-, PRO51592-, PRO1757-, PRO4421-, PRO9903-, PRO1106-, PRO1411-, PRO1486-, PRO1565-, PRO4399- or PRO4404-encoding DNA that is inserted into the vector. The signal sequence may be a prokaryotic signal sequence selected, for example, from the group of the alkaline phosphatase, penicillinase, 1 pp, or heat-stable enterotoxin II leaders. For yeast secretion the signal sequence may be, e.g., the yeast invertase leader, alpha factor leader (including Saccharomyces and Kluyveromyces α-factor leaders, the latter described in U.S. Pat. No. 5,010,182), or acid phosphatase leader, the C. albicans glucoamylase leader (EP 362,179 published 4 Apr. 1990), or the signal described in WO 90/13646 published 15 Nov. 1990. In mammalian cell expression, mammalian signal sequences may be used to direct secretion of the protein, such as signal sequences from secreted polypeptides of the same or related species, as well as viral secretory leaders.

Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV or BPV) are useful for cloning vectors in mammalian cells.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

An example of suitable selectable markers for mammalian cells are those that enable the identification of cells competent to take up the PRO179-, PRO181-, PRO244-, PRO247-, PRO269-, PRO293-, PRO298-, PRO339-, PRO341-, PRO347-, PRO531-, PRO537-, PRO718-, PRO773-, PRO860-, PRO871-, PRO872-, PRO813-, PRO828-, PRO1100-, PRO1114-, PRO1115-, PRO1126-, PRO1133-, PRO1154-, PRO1185-, PRO1194-, PRO1287-, PRO1291-, PRO1293-, PRO1310-, PRO1312-, PRO1335-, PRO1339-, PRO2155-, PRO1356-, PRO1385-, PRO1412-, PRO1487-, PRO1758-, PRO1779-, PRO1785-, PRO1889-, PRO90318-, PRO3434-, PRO3579-, PRO4322-, PRO4343-, PRO4347-, PRO4403-, PRO4976-, PRO260-, PRO6014-, PRO6027-, PRO6181-, PRO6714-, PRO9922-, PRO7179-, PRO7476-, PRO9824-, PRO19814-, PRO19836-, PRO20088-, PRO70789-, PRO50298-, PRO51592-, PRO1757-, PRO4421-, PRO9903-, PRO1106-, PRO1411-, PRO1486-, PRO1565-, PRO4399- or PRO4404-encoding nucleic acid, such as DHFR or thymidine kinase. An appropriate host cell when wild-type DHFR is employed is the CHO cell line deficient in DHFR activity, prepared and propagated as described by Urlaub et al., Proc. Natl. Acad. Sci. USA, 77:4216 (1980). A suitable selection gene for use in yeast is the trp1 gene present in the yeast plasmid YRp7 [Stinchcomb et al., Nature, 282:39 (1979); Kingsman et al., Gene, 7:141 (1979); Tschemper et al., Gene, 10:157 (1980)]. The trp1 gene provides a selection marker for a mutant strain of yeast lacking the ability to grow in tryptophan, for example, ATCC No. 44076 or PEP4-1 [Jones, Genetics, 85:12 (1977)].

Expression and cloning vectors usually contain a promoter operably linked to the PRO179-, PRO181-, PRO244-, PRO247-, PRO269-, PRO293-, PRO298-, PRO339-, PRO341-, PRO347-, PRO531-, PRO537-, PRO718-, PRO773-, PRO860-, PRO871-, PRO872-, PRO813-, PRO828-, PRO1100-, PRO1114-, PRO1115-, PRO1126-, PRO1133-, PRO1154-, PRO1185-, PRO1194-, PRO1287-, PRO1291-, PRO1293-, PRO1310-, PRO1312-, PRO1335-, PRO1339-, PRO2155-, PRO1356-, PRO1385-, PRO1412-, PRO1487-, PRO1758-, PRO1779-, PRO1785-, PRO1889-, PRO90318-, PRO3434-, PRO3579-, PRO4322-, PRO4343-, PRO4347-, PRO4403-, PRO4976-, PRO260-, PRO6014-, PRO6027-, PRO6181-, PRO6714-, PRO9922-, PRO7179-, PRO7476-, PRO9824-, PRO19814-, PRO19836-, PRO20088-, PRO70789-, PRO50298-, PRO51592-, PRO1757-, PRO4421-, PRO9903-, PRO1106-, PRO1411-, PRO1486-, PRO1565-, PRO4399- or PRO4404-encoding nucleic acid sequence to direct mRNA synthesis. Promoters recognized by a variety of potential host cells are well known. Promoters suitable for use with prokaryotic hosts include the β-lactamase and lactose promoter systems [Chang et al., Nature, 275:615 (1978); Goeddel et al., Nature, 281:544 (1979)], alkaline phosphatase, a tryptophan (trp) promoter system [Goeddel, Nucleic Acids Res., 8:4057 (1980); EP 36,776], and hybrid promoters such as the tac promoter [deBoer et al., Proc. Natl. Acad. Sci. USA, 80:21-25 (1983)]. Promoters for use in bacterial systems also will contain a Shine-Dalgarno (S.D.) sequence operably linked to the DNA encoding PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides.

Examples of suitable promoting sequences for use with yeast hosts include the promoters for 3-phosphoglycerate kinase [Hitzeman et al., J. Biol. Chem., 255:2073 (1980)] or other glycolytic enzymes [Hess et al., J. Adv. Enzyme Reg., 7:149 (1968); Holland, Biochemistry, 17:4900 (1978)], such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 transcription from vectors in mammalian host cells is controlled, for example, by promoters obtained from the genomes of viruses such as polyoma virus, fowlpox virus (UK 2,211,504 published 5 Jul. 1989), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of a DNA encoding the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide by higher eukaryotes may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 coding sequence, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides.

Still other methods, vectors, and host cells suitable for adaptation to the synthesis of PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides in recombinant vertebrate cell culture are described in Gething et al., Nature, 293:620-625 (1981); Mantei et al., Nature, 281:40-46 (1979); EP 117,060; and EP 117,058.

4. Detecting Gene Amplification/Expression

Gene amplification and/or expression may be measured in a sample directly, for example, by conventional Southern blotting, Northern blotting to quantitate the transcription of mRNA [Thomas, Proc. Natl. Acad. Sci. USA, 77:5201-5205 (1980)], dot blotting (DNA analysis), or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

Gene expression, alternatively, may be measured by immunological methods, such as immunohistochemical staining of cells or tissue sections and assay of cell culture or body fluids, to quantitate directly the expression of gene product. Antibodies useful for immunohistochemical staining and/or assay of sample fluids may be either monoclonal or polyclonal, and may be prepared in any mammal. Conveniently, the antibodies may be prepared against a native sequence PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide or against a synthetic peptide based on the DNA sequences provided herein or against exogenous sequence fused to PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 DNA and encoding a specific antibody epitope.

5. Purification of Polypeptide

Forms of PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides may be recovered from culture medium or from host cell lysates. If membrane-bound, it can be released from the membrane using a suitable detergent solution (e.g. Triton-X 100) or by enzymatic cleavage. Cells employed in expression of PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides can be disrupted by various physical or chemical means, such as freeze-thaw cycling, sonication, mechanical disruption, or cell lysing agents.

It may be desired to purify PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide. Various methods of protein purification may be employed and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the particular PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide produced.

E. Uses for PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 Polypeptides

Nucleotide sequences (or their complement) encoding PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides have various applications in the art of molecular biology, including uses as hybridization probes, in chromosome and gene mapping and in the generation of anti-sense RNA and DNA. PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 nucleic acid will also be useful for the preparation of PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides by the recombinant techniques described herein.

The full-length native sequence PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 gene, or portions thereof, may be used as hybridization probes for a cDNA library to isolate the full-length PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 cDNA or to isolate still other cDNAs (for instance, those encoding naturally-occurring variants of PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides or PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides from other species) which have a desired sequence identity to the native PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 sequence disclosed herein. Optionally, the length of the probes will be about 20 to about 50 bases. The hybridization probes may be derived from at least partially novel regions of the full length native nucleotide sequence wherein those regions may be determined without undue experimentation or from genomic sequences including promoters, enhancer elements and introns of native sequence PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404. By way of example, a screening method will comprise isolating the coding region of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 gene using the known DNA sequence to synthesize a selected probe of about 40 bases. Hybridization probes may be labeled by a variety of labels, including radionucleotides such as ³²P or ³⁵S, or enzymatic labels such as alkaline phosphatase coupled to the probe via avidin biotin coupling systems. Labeled probes having a sequence complementary to that of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 gene of the present invention can be used to screen libraries of human cDNA, genomic DNA or mRNA to determine which members of such libraries the probe hybridizes to. Hybridization techniques are described in further detail in the Examples below.

Any EST sequences disclosed in the present application may similarly be employed as probes, using the methods disclosed herein.

Other useful fragments of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 nucleic acids include antisense or sense oligonucleotides comprising a singe-stranded nucleic acid sequence (either RNA or DNA) capable of binding to target PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 mRNA (sense) or PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 DNA (antisense) sequences. Antisense or sense oligonucleotides, according to the present invention, comprise a fragment of the coding region of PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 DNA. Such a fragment generally comprises at least about 14 nucleotides, preferably from about 14 to 30 nucleotides. The ability to derive an antisense or a sense oligonucleotide, based upon a cDNA sequence encoding a given protein is described in, for example, Stein and Cohen (Cancer Res. 48:2659, 1988) and van der Krol et al. (BioTechniques 6:958, 1988).

Binding of antisense or sense oligonucleotides to target nucleic acid sequences results in the formation of duplexes that block transcription or translation of the target sequence by one of several means, including enhanced degradation of the duplexes, premature termination of transcription or translation, or by other means. The antisense oligonucleotides thus may be used to block expression of PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404. Antisense or sense oligonucleotides further comprise oligonucleotides having modified sugar-phosphodiester backbones (or other sugar linkages, such as those described in WO 91/06629) and wherein such sugar linkages are resistant to endogenous nucleases. Such oligonucleotides with resistant sugar linkages are stable in vivo (i.e., capable of resisting enzymatic degradation) but retain sequence specificity to be able to bind to target nucleotide sequences.

Other examples of sense or antisense oligonucleotides include those oligonucleotides which are covalently linked to organic moieties, such as those described in WO 90/10048, and other moieties that increases affinity of the oligonucleotide for a target nucleic acid sequence, such as poly-(L-lysine). Further still, intercalating agents, such as ellipticine, and alkylating agents or metal complexes may be attached to sense or antisense oligonucleotides to modify binding specificities of the antisense or sense oligonucleotide for the target nucleotide sequence.

Antisense or sense oligonucleotides may be introduced into a cell containing the target nucleic acid sequence by any gene transfer method, including, for example, CaPO₄-mediated DNA transfection, electroporation, or by using gene transfer vectors such as Epstein-Barr virus. In a preferred procedure, an antisense or sense oligonucleotide is inserted into a suitable retroviral vector. A cell containing the target nucleic acid sequence is contacted with the recombinant retroviral vector, either in vivo or ex vivo. Suitable retroviral vectors include, but are not limited to, those derived from the murine retrovirus M-MuLV, N2 (a retrovirus derived from M-MuLV), or the double copy vectors designated DCT5A, DCT5B and DCT5C (see WO 90/13641).

Sense or antisense oligonucleotides also may be introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell.

Alternatively, a sense or an antisense oligonucleotide may be introduced into a cell containing the target nucleic acid sequence by formation of an oligonucleotide-lipid complex, as described in WO 90/10448. The sense or antisense oligonucleotide-lipid complex is preferably dissociated within the cell by an endogenous lipase.

Antisense or sense RNA or DNA molecules are generally at least about 5 bases in length, about 10 bases in length, about 15 bases in length, about 20 bases in length, about 25 bases in length, about 30 bases in length, about 35 bases in length, about 40 bases in length, about 45 bases in length, about 50 bases in length, about 55 bases in length, about 60 bases in length, about 65 bases in length, about 70 bases in length, about 75 bases in length, about 80 bases in length, about 85 bases in length, about 90 bases in length, about 95 bases in length, about 100 bases in length, or more.

The probes may also be employed in PCR techniques to generate a pool of sequences for identification of closely related PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 coding sequences.

Nucleotide sequences encoding a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide can also be used to construct hybridization probes for mapping the gene which encodes that PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide and for the genetic analysis of individuals with genetic disorders. The nucleotide sequences provided herein may be mapped to a chromosome and specific regions of a chromosome using known techniques, such as in situ hybridization, linkage analysis against known chromosomal markers, and hybridization screening with libraries.

When the coding sequences for PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 encode a protein which binds to another protein (for example, where the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 is a receptor), the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide can be used in assays to identify the other proteins or molecules involved in the binding interaction. By such methods, inhibitors of the receptor/ligand binding interaction can be identified. Proteins involved in such binding interactions can also be used to screen for peptide or small molecule inhibitors or agonists of the binding interaction. Also, the receptor PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 can be used to isolate correlative ligand(s). Screening assays can be designed to find lead compounds that mimic the biological activity of a native PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide or a receptor for PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides. Such screening assays will include assays amenable to high-throughput screening of chemical libraries, making them particularly suitable for identifying small molecule drug candidates. Small molecules contemplated include synthetic organic or inorganic compounds. The assays can be performed in a variety of formats, including protein-protein binding assays, biochemical screening assays, immunoassays and cell based assays, which are well characterized in the art.

Nucleic acids which encode PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides or its modified forms can also be used to generate either transgenic animals or “knock out” animals which, in turn, are useful in the development and screening of therapeutically useful reagents. A transgenic animal (e.g., a mouse or rat) is an animal having cells that contain a transgene, which transgene was introduced into the animal or an ancestor of the animal at a prenatal, e.g., an embryonic stage. A transgene is a DNA which is integrated into the genome of a cell from which a transgenic animal develops. The invention provides cDNA encoding a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide which can be used to clone genomic DNA encoding a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide in accordance with established techniques and the genomic sequences used to generate transgenic animals that contain cells which express DNA encoding PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides. Any technique known in the art may be used to introduce a target gene transgene into animals to produce the founder lines of transgenic animals. Such techniques include, but are not limited to pronuclear microinjection (U.S. Pat. Nos. 4,873,191, 4,736,866 and 4,870,009); retrovirus mediated gene transfer into germ lines (Van der Putten, et al., Proc. Natl. Acad. Sci., USA, 82:6148-6152 (1985)); gene targeting in embryonic stem cells (Thompson, et al., Cell, 56:313-321 (1989)); nonspecific insertional inactivation using a gene trap vector (U.S. Pat. No. 6,436,707); electroporation of embryos (Lo, Mol. Cell. Biol., 3:1803-1814 (1983)); and sperm-mediated gene transfer (Lavitrano, et al., Cell, 57:717-723 (1989)); etc. Typically, particular cells would be targeted for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 transgene incorporation with tissue-specific enhancers. Transgenic animals that include a copy of a transgene encoding a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide introduced into the germ line of the animal at an embryonic stage can be used to examine the effect of increased expression of DNA encoding PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides. Such animals can be used as tester animals for reagents thought to confer protection from, for example, pathological conditions associated with its overexpression. In accordance with this facet of the invention, an animal is treated with the reagent and a reduced incidence of the pathological condition, compared to untreated animals bearing the transgene, would indicate a potential therapeutic intervention for the pathological condition. Alternatively, non-human homologues of PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides can be used to construct a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 “knock out” animal which has a defective or altered gene encoding PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 proteins as a result of homologous recombination between the endogenous gene encoding PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides and altered genomic DNA encoding PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides introduced into an embryonic stem cell of the animal. Preferably the knock out animal is a mammal. More preferably, the mammal is a rodent such as a rat or mouse. For example, cDNA encoding PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides can be used to clone genomic DNA encoding PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides in accordance with established techniques. A portion of the genomic DNA encoding the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide can be deleted or replaced with another gene, such as a gene encoding a selectable marker which can be used to monitor integration. Typically, several kilobases of unaltered flanking DNA (both at the 5′ and 3′ ends) are included in the vector [see e.g., Thomas and Capecchi, Cell, 51:503 (1987) for a description of homologous recombination vectors]. The vector is introduced into an embryonic stem cell line (e.g., by electroporation) and cells in which the introduced DNA has homologously recombined with the endogenous DNA are selected [see e.g., Li et al., Cell, 69:915 (1992)]. The selected cells are then injected into a blastocyst of an animal (e.g., a mouse or rat) to form aggregation chimeras [see e.g., Bradley, in Teratocarcinomas and Embryonic Stem Cells: A Practical Approach, E. J. Robertson, ed. (IRE, Oxford, 1987), pp. 113-152]. A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knockout animals can be characterized for instance, for their ability to defend against certain pathological conditions and for their development of pathological conditions due to absence of the gene encoding the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide.

In addition, knockout mice can be highly informative in the discovery of gene function and pharmaceutical utility for a drug target, as well as in the determination of the potential on-target side effects associated with a given target. Gene function and physiology are so well conserved between mice and humans., since they are both mammals and contain similar numbers of genes, which are highly conserved between the species. It has recently been well documented, for example, that 98% of genes on mouse chromosome 16 have a human ortholog (Mural et al., Science 296:1661-71 (2002)).

Although gene targeting in embryonic stem (ES) cells has enabled the construction of mice with null mutations in many genes associated with human disease, not all genetic diseases are attributable to null mutations. One can design valuable mouse models of human diseases by establishing a method for gene replacement (knock-in) which will disrupt the mouse locus and introduce a human counterpart with mutation, Subsequently one can conduct in vivo drug studies targeting the human protein (Kitamoto et. Al., Biochemical and Biophysical Res. Commun., 222:742-47 (1996)).

Nucleic acid encoding the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides may also be used in gene therapy. In gene therapy applications, genes are introduced into cells in order to achieve in vivo synthesis of a therapeutically effective genetic product, for example for replacement of a defective gene. “Gene therapy” includes both conventional gene therapy where a lasting effect is achieved by a single treatment, and the administration of gene therapeutic agents, which involves the one time or repeated administration of a therapeutically effective DNA or mRNA. Antisense RNAs and DNAs can be used as therapeutic agents for blocking the expression of certain genes in vivo. It has already been shown that short antisense oligonucleotides can be imported into cells where they act as inhibitors, despite their low intracellular concentrations caused by their restricted uptake by the cell membrane. (Zamecnik et al., Proc. Natl. Acad. Sci. USA 83:4143-4146 [1986]). The oligonucleotides can be modified to enhance their uptake, e.g. by substituting their negatively charged phosphodiester groups by uncharged groups.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection (Dzau et al., Trends in Biotechnology 11, 205-210 [1993]). In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262, 4429-4432 (1987); and Wagner et al., Proc. Natl. Acad. Sci. USA 87, 3410-3414 (1990). For review of gene marking and gene therapy protocols see Anderson et al., Science 256, 808-813 (1992).

The PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides described herein may also be employed as molecular weight markers for protein electrophoresis purposes and the isolated nucleic acid sequences may be used for recombinantly expressing those markers.

The nucleic acid molecules encoding the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides or fragments thereof described herein are useful for chromosome identification. In this regard, there exists an ongoing need to identify new chromosome markers, since relatively few chromosome marking reagents, based upon actual sequence data are presently available. Each PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 nucleic acid molecule of the present invention can be used as a chromosome marker.

The PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides and nucleic acid molecules of the present invention may also be used diagnostically for tissue typing, wherein the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides of the present invention may be differentially expressed in one tissue as compared to another, preferably in a diseased tissue as compared to a normal tissue of the same tissue type. PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 nucleic acid molecules will find use for generating probes for PCR, Northern analysis, Southern analysis and Western analysis.

The PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides described herein may also be employed as therapeutic agents. The PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides of the present invention can be formulated according to known methods to prepare pharmaceutically useful compositions, whereby the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 product hereof is combined in admixture with a pharmaceutically acceptable carrier vehicle. Therapeutic formulations are prepared for storage by mixing the active ingredient having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients or stabilizers are nontoxic to recipients at the dosages and concentrations employed, and include buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine or lysine; monosaccharides, disaccharides and other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™ or PEG.

The formulations to be used for in vivo administration must be sterile. This is readily accomplished by filtration through sterile filtration membranes, prior to or following lyophilization and reconstitution.

Therapeutic compositions herein generally are placed into a container having a sterile access port, for example, an intravenous solution bag or vial having a stopper pierceable by a hypodermic injection needle.

The route of administration is in accord with known methods, e.g. injection or infusion by intravenous, intraperitoneal, intracerebral, intramuscular, intraocular, intraarterial or intralesional routes, topical administration, or by sustained release systems.

Dosages and desired drug concentrations of pharmaceutical compositions of the present invention may vary depending on the particular use envisioned. The determination of the appropriate dosage or route of administration is well within the skill of an ordinary physician. Animal experiments provide reliable guidance for the determination of effective doses for human therapy. Interspecies scaling of effective doses can be performed following the principles laid down by Mordenti, J. and Chappell, W. “The use of interspecies scaling in toxicokinetics” In Toxicokinetics and New Drug Development, Yacobi et al., Eds., Pergamon Press, New York 1989, pp. 42-96.

When in vivo administration of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide or agonist or antagonist thereof is employed, normal dosage amounts may vary from about 10 ng/kg to up to 100 mg/kg of mammal body weight or more per day, preferably about 1 μg/kg/day to 10 mg/kg/day, depending upon the route of administration. Guidance as to particular dosages and methods of delivery is provided in the literature; see, for example, U.S. Pat. Nos. 4,657,760; 5,206,344; or 5,225,212. It is anticipated that different formulations will be effective for different treatment compounds and different disorders, that administration targeting one organ or tissue, for example, may necessitate delivery in a manner different from that to another organ or tissue.

Where sustained-release administration of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide is desired in a formulation with release characteristics suitable for the treatment of any disease or disorder requiring administration of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, microencapsulation of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide is contemplated. Microencapsulation of recombinant proteins for sustained release has been successfully performed with human growth hormone (rhGH), interferon-(rhIFN-), interleukin-2, and MN rgp120. Johnson et al., Nat. Med., 2:795-799 (1996); Yasuda, Biomed. Ther., 27:1221-1223 (1993); Hora et al., Bio/Technology, 8:755-758 (1990); Cleland, “Design and Production of Single Immunization Vaccines Using Polylactide Polyglycolide Microsphere Systems,” in Vaccine Design: The Subunit and Adjuvant Approach, Powell and Newman, eds, (Plenum Press: New York, 1995), pp. 439-462; WO 97/03692, WO 96/40072, WO 96/07399; and U.S. Pat. No. 5,654,010.

The sustained-release formulations of these proteins were developed using poly-lactic-coglycolic acid (PLGA) polymer due to its bio compatibility and wide range of biodegradable properties. The degradation products of PLGA, lactic and glycolic acids, can be cleared quickly within the human body. Moreover, the degradability of this polymer can be adjusted from months to years depending on its molecular weight and composition. Lewis, “Controlled release of bioactive agents from lactide/glycolide polymer,” in: M. Chasin and R. Langer (Eds.), Biodegradable Polymers as Drug Delivery Systems (Marcel Dekker: New York, 1990), pp. 1-41.

This invention encompasses methods of screening compounds to identify those that mimic the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide (agonists) or prevent the effect of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide (antagonists). Agonists that mimic a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide would be especially valuable therapeutically in those instances where a negative phenotype is observed based on findings with the non-human transgenic animal whose genome comprises a disruption of the gene which encodes for the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide. Antagonists that prevent the effects of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide would be especially valuable therapeutically in those instances where a positive phenotype is observed based upon observations with the non-human transgenic knockout animal. Screening assays for antagonist drug candidates are designed to identify compounds that bind or complex with the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide encoded by the genes identified herein, or otherwise interfere with the interaction of the encoded polypeptide with other cellular proteins. Such screening assays will include assays amenable to high-throughput screening of chemical libraries, making them particularly suitable for identifying small molecule drug candidates.

The assays can be performed in a variety of formats, including protein-protein binding assays, biochemical screening assays, immunoassays, and cell-based assays, which are well characterized in the art.

All assays for antagonists are common in that they call for contacting the drug candidate with a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide encoded by a nucleic acid identified herein under conditions and for a time sufficient to allow these two components to interact.

In binding assays, the interaction is binding and the complex formed can be isolated or detected in the reaction mixture. The PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide encoded by the gene identified herein or the drug candidate is immobilized on a solid phase, e.g., on a microtiter plate, by covalent or non-covalent attachments. Non-covalent attachment generally is accomplished by coating the solid surface with a solution of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide and drying. Alternatively, an immobilized antibody, e.g., a monoclonal antibody, specific for the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide to be immobilized can be used to anchor it to a solid surface. The assay is performed by adding the non-immobilized component, which may be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labeled antibody specifically binding the immobilized complex.

If the candidate compound interacts with but does not bind to a particular PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide encoded by a gene identified herein, its interaction with that polypeptide can be assayed by methods well known for detecting protein-protein interactions. Such assays include traditional approaches, such as, e.g., cross-linking, co-immunoprecipitation, and co-purification through gradients or chromatographic columns. In addition, protein-protein interactions can be monitored by using a yeast-based genetic system described by Fields and co-workers (Fields and Song, Nature (London), 340:245-246 (1989); Chien et al., Proc. Natl. Acad. Sci. USA, 88:9578-9582 (1991)) as disclosed by Chevray and Nathans, Proc. Natl. Acad. Sci. USA, 89: 5789-5793 (1991). Many transcriptional activators, such as yeast GAL4, consist of two physically discrete modular domains, one acting as the DNA-binding domain, the other one functioning as the transcription-activation domain. The yeast expression system described in the foregoing publications (generally referred to as the “two-hybrid system”) takes advantage of this property, and employs two hybrid proteins, one in which the target protein is fused to the DNA-binding domain of GAL4, and another, in which candidate activating proteins are fused to the activation domain. The expression of a GAL1-lacZ reporter gene under control of a GAL4-activated promoter depends on reconstitution of GAL4 activity via protein-protein interaction. Colonies containing interacting polypeptides are detected with a chromogenic substrate for β-galactosidase. A complete kit (MATCHMAKER™) for identifying protein-protein interactions between two specific proteins using the two-hybrid technique is commercially available from Clontech. This system can also be extended to map protein domains involved in specific protein interactions as well as to pinpoint amino acid residues that are crucial for these interactions.

Compounds that interfere with the interaction of a gene encoding a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide identified herein and other intra- or extracellular components can be tested as follows: usually a reaction mixture is prepared containing the product of the gene and the intra- or extracellular component under conditions and for a time allowing for the interaction and binding of the two products. To test the ability of a candidate compound to inhibit binding, the reaction is run in the absence and in the presence of the test compound. In addition, a placebo may be added to a third reaction mixture, to serve as positive control. The binding (complex formation) between the test compound and the intra- or extracellular component present in the mixture is monitored as described hereinabove. The formation of a complex in the control reaction(s) but not in the reaction mixture containing the test compound indicates that the test compound interferes with the interaction of the test compound and its reaction partner.

To assay for antagonists, the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide may be added to a cell along with the compound to be screened for a particular activity and the ability of the compound to inhibit the activity of interest in the presence of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide indicates that the compound is an antagonist to the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide. Alternatively, antagonists may be detected by combining the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide and a potential antagonist with membrane-bound PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide receptors or recombinant receptors under appropriate conditions for a competitive inhibition assay. The PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide can be labeled, such as by radioactivity, such that the number of PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide molecules bound to the receptor can be used to determine the effectiveness of the potential antagonist. The gene encoding the receptor can be identified by numerous methods known to those of skill in the art, for example, ligand panning and FACS sorting. Coligan et al., Current Protocols in Immun., 1(2): Chapter 5 (1991). Preferably, expression cloning is employed wherein polyadenylated RNA is prepared from a cell responsive to the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide and a cDNA library created from this RNA is divided into pools and used to transfect COS cells or other cells that are not responsive to the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide. Transfected cells that are grown on glass slides are exposed to labeled PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide. The PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide can be labeled by a variety of means including iodination or inclusion of a recognition site for a site-specific protein kinase. Following fixation and incubation, the slides are subjected to autoradiographic analysis. Positive pools are identified and sub-pools are prepared and re-transfected using an interactive sub-pooling and re-screening process, eventually yielding a single clone that encodes the putative receptor.

As an alternative approach for receptor identification, the labeled PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide can be photo affinity-linked with cell membrane or extract preparations that express the receptor molecule. Cross-linked material is resolved by PAGE and exposed to X-ray film. The labeled complex containing the receptor can be excised, resolved into peptide fragments, and subjected to protein micro-sequencing. The amino acid sequence obtained from micro-sequencing would be used to design a set of degenerate oligonucleotide probes to screen a cDNA library to identify the gene encoding the putative receptor.

Another approach in assessing the effect of an antagonist to a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, would be administering a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 antagonist to a wild-type mouse in order to mimic a known knockout phenotype. Thus, one would initially knockout the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 gene of interest and observe the resultant phenotype as a consequence of knocking out or disrupting the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 gene. Subsequently, one could then assess the effectiveness of an antagonist to the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide by administering an antagonist to the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide to a wild-type mouse. An effective antagonist would be expected to mimic the phenotypic effect that was initially observed in the knockout animal.

Likewise, one could assess the effect of an agonist to a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, by administering a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 agonist to a non-human transgenic mouse in order to ameliorate a known negative knockout phenotype. Thus, one would initially knockout the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 gene of interest and observe the resultant phenotype as a consequence of knocking out or disrupting the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 gene. Subsequently, one could then assess the effectiveness of an agonist to the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide by administering an agonist to the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide to a the non-human transgenic mouse. An effective agonist would be expected to ameliorate the negative phenotypic effect that was initially observed in the knockout animal.

In another assay for antagonists, mammalian cells or a membrane preparation expressing the receptor would be incubated with a labeled PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide in the presence of the candidate compound. The ability of the compound to enhance or block this interaction could then be measured.

More specific examples of potential antagonists include an oligonucleotide that binds to the fusions of immunoglobulin with the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, and, in particular, antibodies including, without limitation, poly- and monoclonal antibodies and antibody fragments, single-chain antibodies, anti-idiotypic antibodies, and chimeric or humanized versions of such antibodies or fragments, as well as human antibodies and antibody fragments. Alternatively, a potential antagonist may be a closely related protein, for example, a mutated form of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide that recognizes the receptor but imparts no effect, thereby competitively inhibiting the action of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide.

Another potential PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide antagonist is an antisense RNA or DNA construct prepared using antisense technology, where, e.g., an antisense RNA or DNA molecule acts to block directly the translation of mRNA by hybridizing to targeted mRNA and preventing protein translation. Antisense technology can be used to control gene expression through triple-helix formation or antisense DNA or RNA, both of which methods are based on binding of a polynucleotide to DNA or RNA. For example, the 5′ coding portion of the polynucleotide sequence, which encodes the mature PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides herein, is used to design an antisense RNA oligonucleotide of from about 10 to 40 base pairs in length. A DNA oligonucleotide is designed to be complementary to a region of the gene involved in transcription (triple helix—see Lee et al., Nucl. Acids Res., 6:3073 (1979); Cooney et al., Science, 241: 456 (1988); Dervan et al., Science, 251:1360 (1991)), thereby preventing transcription and the production of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide. The antisense RNA oligonucleotide hybridizes to the mRNA in vivo and blocks translation of the mRNA molecule into the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide (antisense—Okano, Neurochem., 56:560 (1991); Oligodeoxynucleotides as Antisense Inhibitors of Gene Expression (CRC Press: Boca Raton, Fla., 1988). The oligonucleotides described above can also be delivered to cells such that the antisense RNA or DNA may be expressed in vivo to inhibit production of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide. When antisense DNA is used, oligodeoxyribonucleotides derived from the translation-initiation site, e.g., between about −10 and +10 positions of the target gene nucleotide sequence, are preferred.

Potential antagonists include small molecules that bind to the active site, the receptor binding site, or growth factor or other relevant binding site of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, thereby blocking the normal biological activity of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide. Examples of small molecules include, but are not limited to, small peptides or peptide-like molecules, preferably soluble peptides, and synthetic non-peptidyl organic or inorganic compounds.

Ribozymes are enzymatic RNA molecules capable of catalyzing the specific cleavage of RNA. Ribozymes act by sequence-specific hybridization to the complementary target RNA, followed by endonucleolytic cleavage. Specific ribozyme cleavage sites within a potential RNA target can be identified by known techniques. For further details see, e.g., Rossi, Current Biology, 4:469-471 (1994), and PCT publication No. WO 97/33551 (published Sep. 18, 1997).

Nucleic acid molecules in triple-helix formation used to inhibit transcription should be single-stranded and composed of deoxynucleotides. The base composition of these oligonucleotides is designed such that it promotes triple-helix formation via Hoogsteen base-pairing rules, which generally require sizeable stretches of purines or pyrimidines on one strand of a duplex. For further details see, e.g., PCT publication No. WO 97/33551, supra.

These small molecules can be identified by any one or more of the screening assays discussed hereinabove and/or by any other screening techniques well known for those skilled in the art.

Diagnostic and therapeutic uses of the herein disclosed molecules may also be based upon the positive functional assay hits disclosed and described below.

F. Anti-PRO179, Anti-PRO181, Anti-PRO244, Anti-PRO247, Anti-PRO269, Anti-PRO293, Anti-PRO298, Anti-PRO339, Anti-PRO341, Anti-PRO347, Anti-PRO531, Anti-PRO537, Anti-PRO718, Anti-PRO773, Anti-PRO860, Anti-PRO871, Anti-PRO872, Anti-PRO813, Anti-PRO828, Anti-PRO1100, Anti-PRO1114, Anti-PRO1115, Anti-PRO1126, Anti-PRO1133, Anti-PRO1154, Anti-PRO1185, Anti-PRO1194, Anti-PRO1287, Anti-PRO1291, Anti-PRO1293, Anti-PRO1310, Anti-PRO1312, Anti-PRO1335, Anti-PRO1339, Anti-PRO2155, Anti-PRO1356, Anti-PRO1385, Anti-PRO1412, Anti-PRO1487, Anti-PRO1758, Anti-PRO1779, Anti-PRO1785, Anti-PRO1889, Anti-PRO90318, Anti-PRO3434, Anti-PRO3579, Anti-PRO4322, Anti-PRO4343, Anti-PRO4347, Anti-PRO4403, Anti-PRO4976, Anti-PRO260, Anti-PRO6014, Anti-PRO6027, Anti-PRO6181, Anti-PRO6714, Anti-PRO9922, Anti-PRO7179, Anti-PRO7476, Anti-PRO9824, Anti-PRO19814, Anti-PRO19836, Anti-PRO20088, Anti-PRO70789, Anti-PRO50298, Anti-PRO51592, Anti-PRO1757, Anti-PRO4421, Anti-PRO9903, Anti-PRO1106, Anti-PRO1411, Anti-PRO1486, Anti-PRO1565, Anti-PRO4399 or Anti-PRO4404 Antibodies

The present invention provides anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibodies which may find use herein as therapeutic and/or diagnostic agents. Exemplary antibodies include polyclonal, monoclonal, humanized, bispecific, and heteroconjugate antibodies.

1. Polyclonal Antibodies

Polyclonal antibodies are preferably raised in animals by multiple subcutaneous (sc) or intraperitoneal (ip) injections of the relevant antigen and an adjuvant. It may be useful to conjugate the relevant antigen (especially when synthetic peptides are used) to a protein that is immunogenic in the species to be immunized. For example, the antigen can be conjugated to keyhole limpet hemocyanin (KLH), serum albumin, bovine thyroglobulin, or soybean trypsin inhibitor, using a bifunctional or derivatizing agent, e.g., maleimidobenzoyl sulfosuccinimide ester (conjugation through cysteine residues), N-hydroxysuccinimide (through lysine residues), glutaraldehyde, succinic anhydride, SOCl₂, or R′N═C═NR, where R and R¹ are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later, the animals are boosted with ⅕ to 1/10 the original amount of peptide or conjugate in Freund's complete adjuvant by subcutaneous injection at multiple sites. Seven to 14 days later, the animals are bled and the serum is assayed for antibody titer. Animals are boosted until the titer plateaus. Conjugates also can be made in recombinant cell culture as protein fusions. Also, aggregating agents such as alum are suitably used to enhance the immune response.

2. Monoclonal Antibodies

Monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized as described above to elicit lymphocytes that produce or are capable of producing antibodies that will specifically bind to the protein used for immunization. Alternatively, lymphocytes may be immunized in vitro. After immunization, lymphocytes are isolated and then fused with a myeloma cell line using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium which medium preferably contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which substances prevent the growth of HGPRT-deficient cells.

Preferred fusion partner myeloma cells are those that fuse efficiently, support stable high-level production of antibody by the selected antibody-producing cells, and are sensitive to a selective medium that selects against the unfused parental cells. Preferred myeloma cell lines are murine myeloma lines, such as those derived from MOPC-21 and MPC-11 mouse tumors available from the Salk Institute Cell Distribution Center, San Diego, Calif. USA, and SP-2 and derivatives e.g., X63-Ag8-653 cells available from the American Type Culture Collection, Manassas, Va., USA. Human myeloma and mouse-human heteromyeloma cell lines also have been described for the production of human monoclonal antibodies (Kozbor, J. Immunol., 133:3001 (1984); and Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA).

The binding affinity of the monoclonal antibody can, for example, be determined by the Scatchard analysis described in Munson et al., Anal. Biochem., 107:220 (1980).

Once hybridoma cells that produce antibodies of the desired specificity, affinity, and/or activity are identified, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal e.g. by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, hydroxylapatite chromatography, gel electrophoresis, dialysis, etc.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Pliickthun, Immunol. Revs. 130:151-188 (1992).

Monoclonal antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res. 21:2265-2266 (1993)). Thus, these techniques are viable alternatives to traditional monoclonal antibody hybridoma techniques for isolation of monoclonal antibodies.

The DNA that encodes the antibody may be modified to produce chimeric or fusion antibody polypeptides, for example, by substituting human heavy chain and light chain constant domain (C_{H} and C_{L}) sequences for the homologous murine sequences (U.S. Pat. No. 4,816,567; and Morrison, et al., Proc. Natl. Acad. Sci. USA, 81:6851 (1984)), or by fusing the immunoglobulin coding sequence with all or part of the coding sequence for a non-immunoglobulin polypeptide (heterologous polypeptide). The non-immunoglobulin polypeptide sequences can substitute for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

3. Human and Humanized Antibodies

The anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibodies of the invention may further comprise humanized antibodies or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones et al., Nature, 321:522-525 (1986); Riechmann et al., Nature, 332:323-327 (1988); Verhoeyen et al., Science, 239:1534-1536 (1988)], by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity and HAMA response (human anti-mouse antibody) when the antibody is intended for human therapeutic use. According to the so-called “best-fit” method, the sequence of the variable domain of a rodent antibody is screened against the entire library of known human variable domain sequences. The human V domain sequence which is closest to that of the rodent is identified and the human framework region (FR) within it accepted for the humanized antibody (Sims et al., J. Immunol. 151:2296 (1993); Chothia et al., J. Mol. Biol., 196:901 (1987)). Another method uses a particular framework region derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol. 151:2623 (1993)).

It is further important that antibodies be humanized with retention of high binding affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Various forms of a humanized anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody are contemplated. For example, the humanized antibody may be an antibody fragment, such as a Fab, which is optionally conjugated with one or more cytotoxic agent(s) in order to generate an immunoconjugate. Alternatively, the humanized antibody may be an intact antibody, such as an intact IgG1 antibody.

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggemann et al., Year in Immuno. 7:33 (1993); U.S. Pat. Nos. 5,545,806, 5,569,825, 5,591,669 (all of GenPharm); 5,545,807; and WO 97/17852.

Alternatively, phage display technology (McCafferty et al., Nature 348:552-553 [1990]) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson, Kevin S. and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905.

As discussed above, human antibodies may also be generated by in vitro activated B cells (see U.S. Pat. Nos. 5,567,610 and 5,229,275).

4. Antibody Fragments

In certain circumstances there are advantages of using antibody fragments, rather than whole antibodies. The smaller size of the fragments allows for rapid clearance, and may lead to improved access to solid tumors.

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Fab and F(ab′)₂ fragment with increased in vivo half-life comprising a salvage receptor binding epitope residues are described in U.S. Pat. No. 5,869,046. Other techniques for the production of antibody fragments will be apparent to the skilled practitioner. The antibody of choice is a single chain Fv fragment (scFv). See WO 93/16185; U.S. Pat. No. 5,571,894; and U.S. Pat. No. 5,587,458. Fv and sFv are the only species with intact combining sites that are devoid of constant regions; thus, they are suitable for reduced nonspecific binding during in vivo use. sFv fusion proteins may be constructed to yield fusion of an effector protein at either the amino or the carboxy terminus of an sFv. See Antibody Engineering, ed. Borrebaeck, supra. The antibody fragment may also be a “linear antibody”, e.g., as described in U.S. Pat. No. 5,641,870 for example. Such linear antibody fragments may be monospecific or bispecific.

5. Bispecific Antibodies

Bispecific antibodies are antibodies that have binding specificities for at least two different epitopes. Exemplary bispecific antibodies may bind to two different epitopes of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 protein as described herein. Other such antibodies may combine a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 binding site with a binding site for another protein. Alternatively, an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 arm may be combined with an arm which binds to a triggering molecule on a leukocyte such as a T-cell receptor molecule (e.g. CD3), or Fc receptors for IgG (FcγR), such as FcγRI (CD64), FcγRII (CD32) and FcγRIII (CD16), so as to focus and localize cellular defense mechanisms to the PRO179-, PRO181-, PRO244-, PRO247-, PRO269-, PRO293-, PRO298-, PRO339-, PRO341-, PRO347-, PRO531-, PRO537-, PRO718-, PRO773-, PRO860-, PRO871-, PRO872-, PRO813-, PRO828-, PRO1100-, PRO1114-, PRO1115-, PRO1126-, PRO1133-, PRO1154-, PRO1185-, PRO1194-, PRO1287-, PRO1291-, PRO1293-, PRO1310-, PRO1312-, PRO1335-, PRO1339-, PRO2155-, PRO1356-, PRO1385-, PRO1412-, PRO1487-, PRO1758-, PRO1779-, PRO1785-, PRO1889-, PRO90318-, PRO3434-, PRO3579-, PRO4322-, PRO4343-, PRO4347-, PRO4403-, PRO4976-, PRO260-, PRO6014-, PRO6027-, PRO6181-, PRO6714-, PRO9922-, PRO7179-, PRO7476-, PRO9824-, PRO19814-, PRO19836-, PRO20088-, PRO70789-, PRO50298-, PRO51592-, PRO1757-, PRO4421-, PRO9903-, PRO1106-, PRO1411-, PRO1486-, PRO1565-, PRO4399- or PRO4404-expressing cell. Bispecific antibodies may also be used to localize cytotoxic agents to cells which express a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide. These antibodies possess a PRO179-, PRO181-, PRO244-, PRO247-, PRO269-, PRO293-, PRO298-, PRO339-, PRO341-, PRO347-, PRO531-, PRO537-, PRO718-, PRO773-, PRO860-, PRO871-, PRO872-, PRO813-, PRO828-, PRO1100-, PRO1114-, PRO1115-, PRO1126-, PRO1133-, PRO1154-, PRO1185-, PRO1194-, PRO1287-, PRO1291-, PRO1293-, PRO1310-, PRO1312-, PRO1335-, PRO1339-, PRO2155-, PRO1356-, PRO1385-, PRO1412-, PRO1487-, PRO1758-, PRO1779-, PRO1785-, PRO1889-, PRO90318-, PRO3434-, PRO3579-, PRO4322-, PRO4343-, PRO4347-, PRO4403-, PRO4976-, PRO260-, PRO6014-, PRO6027-, PRO6181-, PRO6714-, PRO9922-, PRO7179-, PRO7476-, PRO9824-, PRO19814-, PRO19836-, PRO20088-, PRO70789-, PRO50298-, PRO51592-, PRO1757-, PRO4421-, PRO9903-, PRO1106-, PRO1411-, PRO1486-, PRO1565-, PRO4399- or PRO4404-binding arm and an arm which binds the cytotoxic agent (e.g., saporin, anti-interferon-α, vinca alkaloid, ricin A chain, methotrexate or radioactive isotope hapten). Bispecific antibodies can be prepared as full length antibodies or antibody fragments (e.g., F(ab′)₂ bispecific antibodies).

WO 96/16673 describes a bispecific anti-ErbB2/anti-FcγRIII antibody and U.S. Pat. No. 5,837,234 discloses a bispecific anti-ErbB2/anti-FcγRI antibody. A bispecific anti-ErbB2/Fca antibody is shown in WO98/02463. U.S. Pat. No. 5,821,337 teaches a bispecific anti-ErbB2/anti-CD3 antibody.

Methods for making bispecific antibodies are known in the art. Traditional production of full length bispecific antibodies is based on the co-expression of two immunoglobulin heavy chain-light chain pairs, where the two chains have different specificities (Millstein et al., Nature 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J. 10:3655-3659 (1991).

According to a different approach, antibody variable domains with the desired binding specificity (antibody-antigen combining sites) are fused to immunoglobulin constant domain sequences. Preferably, the fusion is with an Ig heavy chain constant domain, comprising at least part of the hinge, C_{H}2, and C_{H}3 regions. It is preferred to have the first heavy-chain constant region (C_{H}1) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant affect on the yield of the desired chain combination.

The invention provides bispecific antibodies which are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology 121:210 (1986).

According to another approach described in U.S. Pat. No. 5,731,168, the interface between a pair of antibody molecules can be engineered to maximize the percentage of heterodimers which are recovered from recombinant cell culture. The preferred interface comprises at least a part of the C_{H}3 domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent, sodium arsenite, to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Recent progress has facilitated the direct recovery of Fab′-SH fragments from E. coli, which can be chemically coupled to form bispecific antibodies. Shalaby et al., J. Exp. Med. 175: 217-225 (1992) describe the production of a fully humanized bispecific antibody F(ab′)₂molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets. Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a V_{H} connected to a V_{L} by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

Antibodies with more than two valencies are contemplated. For example, trispecific antibodies can be prepared. Tuft et al., J. Immunol. 147:60 (1991).

6. Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells [U.S. Pat. No. 4,676,980], and for treatment of HIV infection [WO 91/00360; WO 92/200373; EP 03089]. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

7. Multivalent Antibodies

A multivalent antibody may be internalized (and/or catabolized) faster than a bivalent antibody by a cell expressing an antigen to which the antibodies bind. The antibodies of the present invention can be multivalent antibodies (which are other than of the IgM class) with three or more antigen binding sites (e.g. tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable domains. For instance, the polypeptide chain(s) may comprise VD1-(X1)ₙ-VD2-(X2)ₙ-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated here comprise alight chain variable domain and, optionally, further comprise a CL domain.

8. Effector Function Engineering

It may be desirable to modify the antibody of the invention with respect to effector function, e.g., so as to enhance antigen-dependent cell-mediated cytotoxicity (ADCC) and/or complement dependent cytotoxicity (CDC) of the antibody. This may be achieved by introducing one or more amino acid substitutions in an Fc region of the antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al., Cancer Research 53:2560-2565 (1993). Alternatively, an antibody can be engineered which has dual Fc regions and may thereby have enhanced complement lysis and ADCC capabilities. See Stevenson et al., Anti-Cancer Drug Design 3:219-230 (1989). To increase the serum half life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

9. Immunoconjugates

The invention also pertains to immunoconjugates comprising an antibody conjugated to a cytotoxic agent such as a chemotherapeutic agent, a growth inhibitory agent, a toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re. Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothio cyanatobenzyl-3-methyldiethylene triaminepentaacetic acid (MX-DTPA) is an exemplary chelating agent for conjugation of radionucleotide to the antibody. See WO94/11026.

Conjugates of an antibody and one or more small molecule toxins, such as a calicheamicin, maytansinoids, a trichothene, and CC1065, and the derivatives of these toxins that have toxin activity, are also contemplated herein.

### Maytansine and Maytansinoids

The invention provides an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody (full length or fragments) which is conjugated to one or more maytansinoid molecules.

Maytansinoids are mitototic inhibitors which act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinol and derivatives and analogues thereof are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533, the disclosures of which are hereby expressly incorporated by reference.

### Maytansinoid-antibody Conjugates

In an attempt to improve their therapeutic index, maytansine and maytansinoids have been conjugated to antibodies specifically binding to tumor cell antigens. Immunoconjugates containing maytansinoids and their therapeutic use are disclosed, for example, in U.S. Pat. Nos. 5,208,020, 5,416,064 and European Patent EP 0 425 235 B1, the disclosures of which are hereby expressly incorporated by reference. Liu et al., Proc. Natl. Acad. Sci. USA 93:8618-8623 (1996) described immunoconjugates comprising a maytansinoid designated DM1 linked to the monoclonal antibody C242 directed against human colorectal cancer. The conjugate was found to be highly cytotoxic towards cultured colon cancer cells, and showed antitumor activity in an in vivo tumor growth assay. Chari et al., Cancer Research 52:127-131 (1992) describe immunoconjugates in which a maytansinoid was conjugated via a disulfide linker to the murine antibody A7 binding to an antigen on human colon cancer cell lines, or to another murine monoclonal antibody TA.1 that binds the HER-2/neu oncogene. The cytotoxicity of the TA.1-maytansonoid conjugate was tested in vitro on the human breast cancer cell line SK-BR-3, which expresses 3×10⁵ HER-2 surface antigens per cell. The drug conjugate achieved a degree of cytotoxicity similar to the free maytansonid drug, which could be increased by increasing the number of maytansinoid molecules per antibody molecule. The A7-maytansinoid conjugate showed low systemic cytotoxicity in mice.

Anti-PRO179, Anti-PRO181, Anti-PRO244, Anti-PRO247, Anti-PRO269, Anti-PRO293, Anti-PRO298, Anti-PRO339, Anti-PRO341, Anti-PRO347, Anti-PRO531, Anti-PRO537, Anti-PRO718, Anti-PRO773, Anti-PRO860, Anti-PRO871, Anti-PRO872, Anti-PRO813, Anti-PRO828, Anti-PRO1100, Anti-PRO1114, Anti-PRO1115, Anti-PRO1126, Anti-PRO1133, Anti-PRO1154, Anti-PRO1185, Anti-PRO1194, Anti-PRO1287, Anti-PRO1291, Anti-PRO1293, Anti-PRO1310, Anti-PRO1312, Anti-PRO1335, Anti-PRO1339, Anti-PRO2155, Anti-PRO1356, Anti-PRO1385, Anti-PRO1412, Anti-PRO1487, Anti-PRO1758, Anti-PRO1779, Anti-PRO1785, Anti-PRO1889, Anti-PRO90318, Anti-PRO3434, Anti-PRO3579, Anti-PRO4322, Anti-PRO4343, Anti-PRO4347, Anti-PRO4403, Anti-PRO4976, Anti-PRO260, Anti-PRO6014, Anti-PRO6027, Anti-PRO6181, Anti-PRO6714, Anti-PRO9922, Anti-PRO7179, Anti-PRO7476, Anti-PRO9824, Anti-PRO19814, Anti-PRO19836, Anti-PRO20088, Anti-PRO70789, Anti-PRO50298, Anti-PRO51592, Anti-PRO1757, Anti-PRO4421, Anti-PRO9903, Anti-PRO1106, Anti-PRO1411, Anti-PRO1486, Anti-PRO1565, Anti-PRO4399 or Anti-PRO4404 Antibody-Maytansinoid Conjugates (Immunoconjugates)

Anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody-maytansinoid conjugates are prepared by chemically linking an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody to a maytansinoid molecule without significantly diminishing the biological activity of either the antibody or the maytansinoid molecule. An average of 3-4 maytansinoid molecules conjugated per antibody molecule has shown efficacy in enhancing cytotoxicity of target cells without negatively affecting the function or solubility of the antibody, although even one molecule of toxin/antibody would be expected to enhance cytotoxicity over the use of naked antibody. Maytansinoids are well known in the art and can be synthesized by known techniques or isolated from natural sources. Suitable maytansinoids are disclosed, for example, in U.S. Pat. No. 5,208,020 and in the other patents and nonpatent publications referred to hereinabove. Preferred maytansinoids are maytansinol and maytansinol analogues modified in the aromatic ring or at other positions of the maytansinol molecule, such as various maytansinol esters.

There are many linking groups known in the art for making antibody-maytansinoid conjugates, including, for example, those disclosed in U.S. Pat. No. 5,208,020 or EP Patent 0 425 235 B1, and Chari et al., Cancer Research 52:127-131 (1992). The linking groups include disufide groups, thioether groups, acid labile groups, photolabile groups, peptidase labile groups, or esterase labile groups, as disclosed in the above-identified patents, disulfide and thioether groups being preferred.

Conjugates of the antibody and maytansinoid may be made using a variety of bifunctional protein coupling agents such as N-succinimidyl-3-(2-pyridyldithio) propionate (SPDP), succinimidyl-4-(N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Particularly preferred coupling agents include N-succinimidyl-3-(2-pyridyldithio) propionate (SPDP) (Carlsson et al., Biochem. J. 173:723-737 [1978]) and N-succinimidyl-4-(2-pyridylthio)pentanoate (SPP) to provide for a disulfide linkage.

The linker may be attached to the maytansinoid molecule at various positions, depending on the type of the link. For example, an ester linkage may be formed by reaction with a hydroxyl group using conventional coupling techniques. The reaction may occur at the C-3 position having a hydroxyl group, the C-14 position modified with hyrdoxymethyl, the C-15 position modified with a hydroxyl group, and the C-20 position having a hydroxyl group. The linkage is formed at the C-3 position of maytansinol or a maytansinol analogue.

### Calicheamicin

Another immunoconjugate of interest comprises an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody conjugated to one or more calicheamicin molecules. The calicheamicin family of antibiotics are capable of producing double-stranded DNA breaks at sub-picomolar concentrations. For the preparation of conjugates of the calicheamicin family, see U.S. Pat. Nos. 5,712,374, 5,714,586, 5,739,116, 5,767,285, 5,770,701, 5,770,710, 5,773,001, 5,877,296 (all to American Cyanamid Company). Structural analogues of calicheamicin which may be used include, but are not limited to, γ₁^{I}, α₂^{I}, α₃^{I}, N-acetyl-γ₁^{I}, PSAG and θ₁^{I} (Hinman et al., Cancer Research 53:3336-3342 (1993), Lode et al., Cancer Research 58:2925-2928 (1998) and the aforementioned U.S. patents to American Cyanamid). Another anti-tumor drug that the antibody can be conjugated is QFA which is an antifolate. Both calicheamicin and QFA have intracellular sites of action and do not readily cross the plasma membrane. Therefore, cellular uptake of these agents through antibody mediated internalization greatly enhances their cytotoxic effects.

### Other Cytotoxic Agents

Other antitumor agents that can be conjugated to the anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibodies of the invention include BCNU, streptozoicin, vincristine and 5-fluorouracil, the family of agents known collectively LL-E33288 complex described in U.S. Pat. Nos. 5,053,394, 5,770,710, as well as esperamicins (U.S. Pat. No. 5,877,296).

Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. See, for example, WO 93/21232 published Oct. 28, 1993.

The present invention further contemplates an immunoconjugate formed between an antibody and a compound with nucleolytic activity (e.g., a ribonuclease or a DNA endonuclease such as a deoxyribonuclease; DNase).

For selective destruction of the tumor, the antibody may comprise a highly radioactive atom. A variety of radioactive isotopes are available for the production of radioconjugated anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibodies. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the conjugate is used for diagnosis, it may comprise a radioactive atom for scintigraphic studies, for example tc^{99m} or I¹²³, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

The radio- or other labels may be incorporated in the conjugate in known ways. For example, the peptide may be biosynthesized or may be synthesized by chemical amino acid synthesis using suitable amino acid precursors involving, for example, fluorine-19 in place of hydrogen. Labels such as tc^{99m} or I¹²³, Re¹⁸⁶, Re¹⁸⁸ and In¹¹¹ can be attached via a cysteine residue in the peptide. Yttrium-90 can be attached via a lysine residue. The IODOGEN method (Fraker et al (1978) Biochem. Biophys. Res. Commun. 80: 49-57 can be used to incorporate iodine-123. “Monoclonal Antibodies in Immuno scintigraphy” (Chatal, CRC Press 1989) describes other methods in detail.

Conjugates of the antibody and cytotoxic agent may be made using a variety of bifunctional protein coupling agents such as N-succinimidyl-3-(2-pyridyldithio) propionate (SPDP), succinimidyl-4-(N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl)hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of the cytotoxic drug in the cell. For example, an acid-labile linker, peptidase-sensitive linker, photolabile linker, dimethyl linker or disulfide-containing linker (Chari et al., Cancer Research 52:127-131 (1992); U.S. Pat. No. 5,208,020) may be used.

Alternatively, a fusion protein comprising the anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate.

The invention provides that the antibody may be conjugated to a “receptor” (such streptavidin) for utilization in tumor pre-targeting wherein the antibody-receptor conjugate is administered to the patient, followed by removal of unbound conjugate from the circulation using a clearing agent and then administration of a “ligand” (e.g., avidin) which is conjugated to a cytotoxic agent (e.g., a radionucleotide).

10. Immunoliposomes

The anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibodies disclosed herein may also be formulated as immunoliposomes. A “liposome” is a small vesicle composed of various types of lipids, phospholipids and/or surfactant which is useful for delivery of a drug to a mammal. The components of the liposome are commonly arranged in a bilayer formation, similar to the lipid arrangement of biological membranes. Liposomes containing the antibody are prepared by methods known in the art, such as described in Epstein et al., Proc. Natl. Acad. Sci. USA 82:3688 (1985); Hwang et al., Proc. Natl. Acad. Sci. USA 77:4030 (1980); U.S. Pat. Nos. 4,485,045 and 4,544,545; and WO97/38731 published Oct. 23, 1997. Liposomes with enhanced circulation time are disclosed in U.S. Pat. No. 5,013,556.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem. 257:286-288 (1982) via a disulfide interchange reaction. A chemotherapeutic agent is optionally contained within the liposome. See Gabizon et al., J. National Cancer Inst. 81(19):1484 (1989).

11. Pharmaceutical Compositions of Antibodies

Antibodies specifically binding a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide identified herein, as well as other molecules identified by the screening assays disclosed hereinbefore, can be administered for the treatment of various disorders in the form of pharmaceutical compositions.

If the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide is intracellular and whole antibodies are used as inhibitors, internalizing antibodies are preferred. However, lipofections or liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993). The formulation herein may also contain more than one active compound as necessary for the particular indication being treated, preferably those with complementary activities that do not adversely affect each other. Alternatively, or in addition, the composition may comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin micro spheres, microemulsions, nano-particles, and nanocapsules) or in macroemulsions. Such techniques are disclosed in Remington's Pharmaceutical Sciences, supra.

The formulations to be used for in vivo administration must be sterile. This is readily accomplished by filtration through sterile filtration membranes.

Sustained-release preparations may be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

G. Uses for Anti-PRO179, Anti-PRO181, Anti-PRO244, Anti-PRO247, Anti-PRO269, Anti-PRO293, Anti-PRO298, Anti-PRO339, Anti-PRO341, Anti-PRO347, Anti-PRO531, Anti-PRO537, Anti-PRO718, Anti-PRO773, Anti-PRO860, Anti-PRO871, Anti-PRO872, Anti-PRO813, Anti-PRO828, Anti-PRO1100, Anti-PRO1114, Anti-PRO1115, Anti-PRO1126, Anti-PRO1133, Anti-PRO1154, Anti-PRO1185, Anti-PRO1194, Anti-PRO1287, Anti-PRO1291, Anti-PRO1293, Anti-PRO1310, Anti-PRO1312, Anti-PRO1335, Anti-PRO1339, Anti-PRO2155, Anti-PRO1356, Anti-PRO1385, Anti-PRO1412, Anti-PRO1487, Anti-PRO1758, Anti-PRO1779, Anti-PRO1785, Anti-PRO1889, Anti-PRO90318, Anti-PRO3434, Anti-PRO3579, Anti-PRO4322, Anti-PRO4343, Anti-PRO4347, Anti-PRO4403, Anti-PRO4976, Anti-PRO260, Anti-PRO6014, Anti-PRO6027, Anti-PRO6181, Anti-PRO6714, Anti-PRO9922, Anti-PRO7179, Anti-PRO7476, Anti-PRO9824, Anti-PRO19814, Anti-PRO19836, Anti-PRO20088, Anti-PRO70789, Anti-PRO50298, Anti-PRO51592, Anti-PRO1757, Anti-PRO4421, Anti-PRO9903, Anti-PRO1106, Anti-PRO1411, Anti-PRO1486, Anti-PRO1565, Anti-PRO4399 or Anti-PRO4404 Antibodies

The anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibodies of the invention have various therapeutic and/or diagnostic utilities for a neurological disorder; a cardiovascular, endothelial or angiogenic disorder; an immunological disorder; an oncological disorder; an embryonic developmental disorder or lethality, or a metabolic abnormality. For example, anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibodies may be used in diagnostic assays for PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404, e.g., detecting its expression (and in some cases, differential expression) in specific cells, tissues, or serum. Various diagnostic assay techniques known in the art may be used, such as competitive binding assays, direct or indirect sandwich assays and immunoprecipitation assays conducted in either heterogeneous or homogeneous phases [Zola, Monoclonal Antibodies: A Manual of Techniques, CRC Press, Inc. (1987) pp. 147-158]. The antibodies used in the diagnostic assays can be labeled with a detectable moiety. The detectable moiety should be capable of producing, either directly or indirectly, a detectable signal. For example, the detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin, or an enzyme, such as alkaline phosphatase, beta-galactosidase or horseradish peroxidase. Any method known in the art for conjugating the antibody to the detectable moiety may be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. and Cytochem., 30:407 (1982).

Anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibodies also are useful for the affinity purification of PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides from recombinant cell culture or natural sources. In this process, the antibodies against PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides are immobilized on a suitable support, such a Sephadex resin or filter paper, using methods well known in the art. The immobilized antibody then is contacted with a sample containing the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide to be purified, and thereafter the support is washed with a suitable solvent that will remove substantially all the material in the sample except the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, which is bound to the immobilized antibody. Finally, the support is washed with another suitable solvent that will release the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide from the antibody.

The following examples are offered for illustrative purposes only, and are not intended to limit the scope of the present invention in any way.

All patent and literature references cited in the present specification are hereby incorporated by reference in their entirety.

### EXAMPLES

Commercially available reagents referred to in the examples were used according to manufacturer's instructions unless otherwise indicated. The source of those cells identified in the following examples, and throughout the specification, by ATCC accession numbers is the American Type Culture Collection, Manassas, Va.

### Example 1

### Extracellular Domain Homology Screening to Identify Novel Polypeptides and cDNA Encoding Therefor

The extracellular domain (ECD) sequences (including the secretion signal sequence, if any) from about 950 known secreted proteins from the Swiss-Prot public database were used to search EST databases. The EST databases included public databases (e.g., Dayhoff, GenBank), and proprietary databases (e.g. LIFE SHP, Incyte Pharmaceuticals, Palo Alto, Calif.). The search was performed using the computer program BLAST or BLAST-2 (Altschul et al., Methods in Enzymology, 266:460-480 (1996)) as a comparison of the ECD protein sequences to a 6 frame translation of the EST sequences. Those comparisons with a BLAST score of 70 (or in some cases 90) or greater that did not encode known proteins were clustered and assembled into consensus DNA sequences with the program “phrap” (Phil Green, University of Washington, Seattle, Wash.).

Using this extracellular domain homology screen, consensus DNA sequences were assembled relative to the other identified EST sequences using phrap. In addition, the consensus DNA sequences obtained were often (but not always) extended using repeated cycles of BLAST or BLAST-2 and phrap to extend the consensus sequence as far as possible using the sources of EST sequences discussed above.

Based upon the consensus sequences obtained as described above, oligonucleotides were then synthesized and used to identify by PCR a cDNA library that contained the sequence of interest and for use as probes to isolate a clone of the full-length coding sequence for a PRO polypeptide. Forward and reverse PCR primers generally range from 20 to 30 nucleotides and are often designed to give a PCR product of about 100-1000 by in length. The probe sequences are typically 40-55 by in length. In some cases, additional oligonucleotides are synthesized when the consensus sequence is greater than about 1-1.5 kbp. In order to screen several libraries for a full-length clone, DNA from the libraries was screened by PCR amplification, as per Ausubel et al., Current Protocols in Molecular Biology, with the PCR primer pair. A positive library was then used to isolate clones encoding the gene of interest using the probe oligonucleotide and one of the primer pairs.

The cDNA libraries used to isolate the cDNA clones were constructed by standard methods using commercially available reagents such as those from Invitrogen, San Diego, Calif. The cDNA was primed with oligo dT containing a NotI site, linked with blunt to SalI hemikinased adaptors, cleaved with NotI, sized appropriately by gel electrophoresis, and cloned in a defined orientation into a suitable cloning vector (such as pRKB or pRKD; pRK5B is a precursor of pRK5D that does not contain the SfiI site; see, Holmes et al., Science, 253:1278-1280 (1991)) in the unique XhoI and NotI sites.

### Example 2

### Isolation of cDNA clones by Amylase Screening

1. Preparation of Oligo dT Primed cDNA Library

mRNA was isolated from a human tissue of interest using reagents and protocols from Invitrogen, San Diego, Calif. (Fast Track 2). This RNA was used to generate an oligo dT primed cDNA library in the vector pRK5D using reagents and protocols from Life Technologies, Gaithersburg, Md. (Super Script Plasmid System). In this procedure, the double stranded cDNA was sized to greater than 1000 by and the SalI/NotI linkered cDNA was cloned into XhoI/Noff cleaved vector. pRK5D is a cloning vector that has an sp6 transcription initiation site followed by an SfiI restriction enzyme site preceding the XhoI/NotI cDNA cloning sites.

2. Preparation of Random Primed cDNA Library

A secondary cDNA library was generated in order to preferentially represent the 5′ ends of the primary cDNA clones. Sp6 RNA was generated from the primary library (described above), and this RNA was used to generate a random primed cDNA library in the vector pSST-AMY.0 using reagents and protocols from Life Technologies (Super Script Plasmid System, referenced above). In this procedure the double stranded cDNA was sized to 500-1000 bp, linkered with blunt to NotI adaptors, cleaved with SfiI, and cloned into SfiI/NotI cleaved vector. pSST-AMY.0 is a cloning vector that has a yeast alcohol dehydrogenase promoter preceding the cDNA cloning sites and the mouse amylase sequence (the mature sequence without the secretion signal) followed by the yeast alcohol dehydrogenase terminator, after the cloning sites. Thus, cDNAs cloned into this vector that are fused in frame with amylase sequence will lead to the secretion of amylase from appropriately transfected yeast colonies.

3. Transformation and Detection

DNA from the library described in paragraph 2 above was chilled on ice to which was added electrocompetent DH10B bacteria (Life Technologies, 20 ml). The bacteria and vector mixture was then electroporated as recommended by the manufacturer. Subsequently, SOC media (Life Technologies, 1 ml) was added and the mixture was incubated at 37° C. for 30 minutes. The transformants were then plated onto 20 standard 150 mm LB plates containing ampicillin and incubated for 16 hours (37° C.). Positive colonies were scraped off the plates and the DNA was isolated from the bacterial pellet using standard protocols, e.g. CsCl-gradient. The purified DNA was then carried on to the yeast protocols below.

The yeast methods were divided into three categories: (1) Transformation of yeast with the plasmid/cDNA combined vector; (2) Detection and isolation of yeast clones secreting amylase; and (3) PCR amplification of the insert directly from the yeast colony and purification of the DNA for sequencing and further analysis.

The yeast strain used was HD56-5A (ATCC-90785). This strain has the following genotype: MAT alpha, ura3-52, leu2-3, leu2-112, his3-11, his3-15, MAL⁺, SUC⁺, GAL⁺. Preferably, yeast mutants can be employed that have deficient post-translational pathways. Such mutants may have translocation deficient alleles in sec71, sec72, sec62, with truncated sec71 being most preferred. Alternatively, antagonists (including antisense nucleotides and/or ligands) which interfere with the normal operation of these genes, other proteins implicated in this post translation pathway (e.g., SEC61p, SEC72p, SEC62p, SEC63p, TDJ1p or SSA1p-4-p) or the complex formation of these proteins may also be preferably employed in combination with the amylase-expressing yeast.

Transformation was performed based on the protocol outlined by Gietz et al., Nucl. Acid. Res., 20:1425 (1992). Transformed cells were then inoculated from agar into YEPD complex media broth (100 ml) and grown overnight at 30° C. The YEPD broth was prepared as described in Kaiser et al ., Methods in Yeast Genetics, Cold Spring Harbor Press, Cold Spring Harbor, N.Y., p. 207 (1994). The overnight culture was then diluted to about 2×10⁶ cells/ml (approx. OD₆₀₀=0.1) into fresh YEPD broth (500 ml) and regrown to 1×10⁷ cells/ml (approx. OD₆₀₀=0.4-0.5).

The cells were then harvested and prepared for transformation by transfer into GS3 rotor bottles in a Sorval GS3 rotor at 5,000 rpm for 5 minutes, the supernatant discarded, and then resuspended into sterile water, and centrifuged again in 50 ml falcon tubes at 3,500 rpm in a Beckman GS-6KR centrifuge. The supernatant was discarded and the cells were subsequently washed with LiAc/TE (10 ml, 10 mM Tris-HCl, 1 mM EDTA pH 7.5, 100 mM Li₂OOCCH₃), and resuspended into LiAc/TE (2.5 ml).

Transformation took place by mixing the prepared cells (100 μl) with freshly denatured single stranded salmon testes DNA (Lofstrand Labs, Gaithersburg, Md.) and transforming DNA (1 μg, vol. <10 μl) in microfuge tubes. The mixture was mixed briefly by vortexing, then 40% PEG/TE (600 μl, 40% polyethylene glycol-4000, 10 mM Tris-HCl, 1 mM EDTA, 100 mM Li₂OOCCH₃, pH 7.5) was added. This mixture was gently mixed and incubated at 30° C. while agitating for 30 minutes. The cells were then heat shocked at 42° C. for 15 minutes, and the reaction vessel centrifuged in a microfuge at 12,000 rpm for 5-10 seconds, decanted and resuspended into TE (500 μl, 10 mM Tris-HCl, 1 mM EDTA pH 7.5) followed by recentrifugation. The cells were then diluted into TE (1 ml) and aliquots (200 μl) were spread onto the selective media previously prepared in 150 mm growth plates (VWR).

Alternatively, instead of multiple small reactions, the transformation was performed using a single, large scale reaction, wherein reagent amounts were scaled up accordingly.

The selective media used was a synthetic complete dextrose agar lacking uracil (SCD-Ura) prepared as described in Kaiser et al., Methods in Yeast Genetics, Cold Spring Harbor Press, Cold Spring Harbor, N.Y., p. 208-210 (1994). Transformants were grown at 30° C. for 2-3 days.

The detection of colonies secreting amylase was performed by including red starch in the selective growth media. Starch was coupled to the red dye (Reactive Red-120, Sigma) as per the procedure described by Biely et al., Anal. Biochem., 172:176-179 (1988). The coupled starch was incorporated into the SCD-Ura agar plates at a final concentration of 0.15% (w/v), and was buffered with potassium phosphate to a pH of 7.0 (50-100 mM final concentration).

The positive colonies were picked and streaked across fresh selective media (onto 150 mm plates) in order to obtain well isolated and identifiable single colonies. Well isolated single colonies positive for amylase secretion were detected by direct incorporation ofred starch into buffered SCD-Ura agar. Positive colonies were determined by their ability to break down starch resulting in a clear halo around the positive colony visualized directly.

4. Isolation of DNA by PCR Amplification

When a positive colony was isolated, a portion of it was picked by a toothpick and diluted into sterile water (30 μl) in a 96 well plate. At this time, the positive colonies were either frozen and stored for subsequent analysis or immediately amplified. An aliquot of cells (5 μl) was used as a template for the PCR reaction in a 25 μl volume containing: 0.5 μl Klentaq (Clontech, Palo Alto, Calif.); 4.0 μl 10 mM dNTP's (Perkin Elmer-Cetus); 2.5 μl Kentaq buffer (Clontech); 0.25 μl forward oligo 1; 0.25 μl reverse oligo 2; 12.5 μl distilled water. The sequence of the forward oligonucleotide 1 was:

(SEQ ID NO: 151)
5′-TGTAAAACGACGGCCAGTTAAATAGACCTGCAATTATTAATCT-3′

The sequence of reverse oligonucleotide 2 was:

(SEQ ID NO: 152)
5′-CAGGAAACAGCTATGACCACCTGCACACCTGCAAATCCATT-3′

PCR was then performed as follows:

| | | | | |
|---|---|---|---|---|
| a. | | Denature | 92° C., | 5 minutes |
| b. | 3 cycles of: | Denature | 92° C., | 30 seconds |
| | | Anneal | 59° C., | 30 seconds |
| | | Extend | 72° C., | 60 seconds |
| c. | 3 cycles of: | Denature | 92° C., | 30 seconds |
| | | Anneal | 57° C., | 30 seconds |
| | | Extend | 72° C., | 60 seconds |
| d. | 25 cycles of: | Denature | 92° C., | 30 seconds |
| | | Anneal | 55° C., | 30 seconds |
| | | Extend | 72° C., | 60 seconds |
| e. | | Hold | 4° C. | |

The underlined regions of the oligonucleotides annealed to the ADH promoter region and the amylase region, respectively, and amplified a 307 by region from vector pSST-AMY.0 when no insert was present. Typically, the first 18 nucleotides of the 5′ end of these oligonucleotides contained annealing sites for the sequencing primers. Thus, the total product of the PCR reaction from an empty vector was 343 bp. However, signal sequence-fused cDNA resulted in considerably longer nucleotide sequences.

Following the PCR, an aliquot of the reaction (5 μl) was examined by agarose gel electrophoresis in a 1% agarose gel using a Tris-Borate-EDTA (TBE) buffering system as described by Sambrook et al., supra. Clones resulting in a single strong PCR product larger than 400 by were further analyzed by DNA sequencing after purification with a 96 Qiaquick PCR clean-up column (Qiagen Inc., Chatsworth, Calif.).

### Example 3

### Isolation of cDNA Clones Using Signal Algorithm Analysis

Various polypeptide-encoding nucleic acid sequences were identified by applying a proprietary signal sequence finding algorithm developed by Genentech, Inc. (South San Francisco, Calif.) upon ESTs as well as clustered and assembled EST fragments from public (e.g., GenBank) and/or private (LIFESEQ®, Incyte Pharmaceuticals, Inc., Palo Alto, Calif.) databases. The signal sequence algorithm computes a secretion signal score based on the character of the DNA nucleotides surrounding the first and optionally the second methionine codon(s) (ATG) at the 5′-end of the sequence or sequence fragment under consideration. The nucleotides following the first ATG must code for at least 35 unambiguous amino acids without any stop codons. If the first ATG has the required amino acids, the second is not examined. If neither meets the requirement, the candidate sequence is not scored. In order to determine whether the EST sequence contains an authentic signal sequence, the DNA and corresponding amino acid sequences surrounding the ATG codon are scored using a set of seven sensors (evaluation parameters) known to be associated with secretion signals. Use of this algorithm resulted in the identification of numerous polypeptide-encoding nucleic acid sequences.

Using the techniques described in Examples 1 to 3 above, numerous full-length cDNA clones were identified as encoding PRO179, PRO181, PRO244, PRO247, PRO269, PRO293, PRO298, PRO339, PRO341, PRO347, PRO531, PRO537, PRO718, PRO773, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO1356, PRO1385, PRO1412, PRO1487, PRO1758, PRO1779, PRO1785, PRO1889, PRO3434, PRO3579, PRO4322, PRO4343, PRO4347, PRO4403, PRO4976, PRO260, PRO6014, PRO6027, PRO6181, PRO6714, PRO7179, PRO7476, PRO19814, PRO20088, PRO1757, PRO4421, PRO9903, PRO1106, PRO1411, PRO1486, PRO1565, PRO4399 or PRO4404 polypeptides as disclosed herein. These cDNAs were then deposited under the terms of the Budapest Treaty with the American Type Culture Collection, University Blvd., Manassas, Va. 20110-2209, USA (ATCC) as shown in Table 7 below. In addition, the sequence of DNA60614 encoding PRO860 polypeptides was identified from GenBank accession no.: AF361473; the sequence of DNA88062 encoding PRO2155 polypeptides was identified from GenBank accession no.: A06977; the sequence of DNA336109 encoding PRO90318 polypeptides was identified from GenBank accession no.: AF417580; the sequence of DNA142524 encoding PRO9922 polypeptides was identified from GenBank accession no.: U88879; the sequence of DNA111030 encoding PRO9824 polypeptides was identified from GenBank accession no.: AF316597; the sequence of DNA144839 encoding PRO19836 polypeptides was identified from GenBank accession no.: BC023567; the sequence of DNA295801 encoding PRO70789 polypeptides was identified from GenBank accession no.: AY260763; the sequence of DNA255219 encoding PRO50298 polypeptides was identified from GenBank accession no.: AK026226; and the sequence of DNA256561 encoding PRO51592 polypeptides was identified from GenBank accession no.: AF001622.

TABLE 7

| Material | ATCC Dep. No. | Deposit Date |
|---|---|---|
| DNA16451-1078 | 209281 | Sept. 18, 1997 |
| DNA23330-1390 | 209775 | Apr. 14, 1998 |
| DNA35668-1171 | 209371 | Oct. 16, 1997 |
| DNA35673-1201 | 209418 | Oct. 28, 1997 |
| DNA38260-1180 | 209397 | Oct. 17, 1997 |
| DNA37151-1193 | 209393 | Oct. 17, 1997 |
| DNA39975-1210 | 209783 | Apr. 21, 1998 |
| DNA43466-1225 | 209490 | Nov. 21, 1997 |
| DNA26288-1239 | 209792 | Apr. 21, 1998 |
| DNA44176-1244 | 209532 | Dec. 10, 1997 |
| DNA48314-1320 | 209702 | Mar. 26, 1998 |
| DNA49141-1431 | 203003 | June 23, 1998 |
| DNA49647-1398 | 209919 | June 2, 1998 |
| DNA48303-2829 | PTA-1342 | Feb. 8, 2000 |
| DNA50919-1361 | 209848 | May 6, 1998 |
| DNA49819-1439 | 209931 | June 2, 1998 |
| DNA57834-1339 | 209954 | June 9, 1998 |
| DNA57037-1444 | 209903 | May 27, 1998 |
| DNA59619-1464 | 203041 | July 1, 1998 |
| DNA57033-1403 | 209905 | May 27, 1998 |
| DNA56868-1478 | 203024 | June 23, 1998 |
| DNA60615-1483 | 209980 | June 16, 1998 |
| DNA53913-1490 | 203162 | Aug. 25, 1998 |
| DNA59846-1503 | 209978 | June 16, 1998 |
| DNA62881-1515 | 203096 | Aug. 4, 1998 |
| DNA57841-1522 | 203458 | Nov. 3, 1998 |
| DNA61755-1554 | 203112 | Aug. 11, 1998 |
| DNA59610-1556 | 209990 | June 16, 1998 |
| DNA60618-1557 | 203292 | Sept. 29, 1998 |
| DNA47394-1572 | 203109 | Aug. 11, 1998 |
| DNA61873-1574 | 203132 | Aug. 18, 1998 |
| DNA62812-1594 | 203248 | Sept. 9, 1998 |
| DNA66669-1597 | 203272 | Sept. 22, 1998 |
| DNA64886-1601 | 203241 | Sept. 9, 1998 |
| DNA68869-1610 | 203164 | Aug. 25, 1998 |
| DNA64897-1628 | 203216 | Sept. 15, 1998 |
| DNA68836-1656 | 203455 | Nov. 3, 1998 |
| DNA76399-1700 | 203472 | Nov. 17, 1998 |
| DNA73775-1707 | PTA-128 | May 25, 1999 |
| DNA80136-2503 | 203541 | Dec. 15, 1998 |
| DNA77623-2524 | 203546 | Dec. 22, 1998 |
| DNA77631-2537 | 203651 | Feb. 9, 1999 |
| DNA68862-2546 | 203652 | Feb. 9, 1999 |
| DNA92223-2567 | 203851 | Mar. 16, 1999 |
| DNA92255-2584 | 203866 | Mar. 23, 1999 |
| DNA92288-2588 | 203892 | Mar. 30, 1999 |
| DNA83509-2612 | 203965 | Apr. 27, 1999 |
| DNA100902-2646 | PTA-42 | May 11, 1999 |
| DNA33470-1175 | 209398 | Oct. 17, 1997 |
| DNA92217-2697 | PTA-513 | Aug. 10, 1999 |
| DNA105838-2702 | PTA-476 | Aug. 3, 1999 |
| DNA107698-2715 | PTA-472 | Aug. 3, 1999 |
| DNA82358-2738 | PTA-510 | Aug. 10, 1999 |
| DNA108701-2749 | PTA-554 | Aug. 17, 1999 |
| DNA115253-2757 | PTA-612 | Aug. 31, 1999 |
| DNA148004-2882 | PTA-1779 | Apr. 25, 2000 |
| DNA150157-2898 | PTA-1777 | Apr. 25, 2000 |
| DNA76398-1699 | 203474 | Nov. 17, 1998 |
| DNA96879-2619 | 203967 | Apr. 27, 1999 |
| DNA119516-2797 | PTA-1083 | Dec. 22, 1999 |
| DNA59609-1470 | 209963 | June 9, 1998 |
| DNA59212-1627 | 203245 | Sept. 9, 1998 |
| DNA71180-1655 | 203403 | Oct. 27, 1998 |
| DNA73727-1673 | 203459 | Nov. 3, 1998 |
| DNA89220-2608 | PTA-130 | May 25, 1999 |
| DNA84142-2613 | PTA-22 | May 4, 1999 |

These deposits were made under the provisions of the Budapest Treaty on the International Recognition of the Deposit of Microorganisms for the Purpose of Patent Procedure and the Regulations thereunder (Budapest Treaty). This assures maintenance of a viable culture of the deposit for 30 years from the date of deposit. The deposits will be made available by ATCC under the terms of the Budapest Treaty, and subject to an agreement between Genentech, Inc. and ATCC, which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 USC §122 and the Commissioner's rules pursuant thereto (including 37 CFR §1.14 with particular reference to 8860G 638).

The assignee of the present application has agreed that if a culture of the materials on deposit should die or be lost or destroyed when cultivated under suitable conditions, the materials will be promptly replaced on notification with another of the same. Availability of the deposited material is not to be construed as a license to practice the invention in contravention of the rights granted under the authority of any government in accordance with its patent laws.

### Example 4

### Isolation of cDNA clones Encoding Human PRO179 Polypeptides [UNQ153]

A cDNA clone (DNA16451-1078) encoding a native human PRO179 polypeptide was identified using a yeast screen, in a human fetal liver library that preferentially represents the 5′ ends of the primary cDNA clones.

The primers used for the identification of DNA16451-1078 are as follows:

OLI114:
(SEQ ID NO: 153)
5′-CCACGTTGGCTTGAAATTGA-3′
OLI115:
(SEQ ID NO: 154)
5′-CCTTTAGAATTGATCAAGACAATTCATGATTTGATTCTCTATCTCC
AGAG-3′
OLI116:
(SEQ ID NO: 155)
5′-TCGTCTAACATAGCAAATC-3′

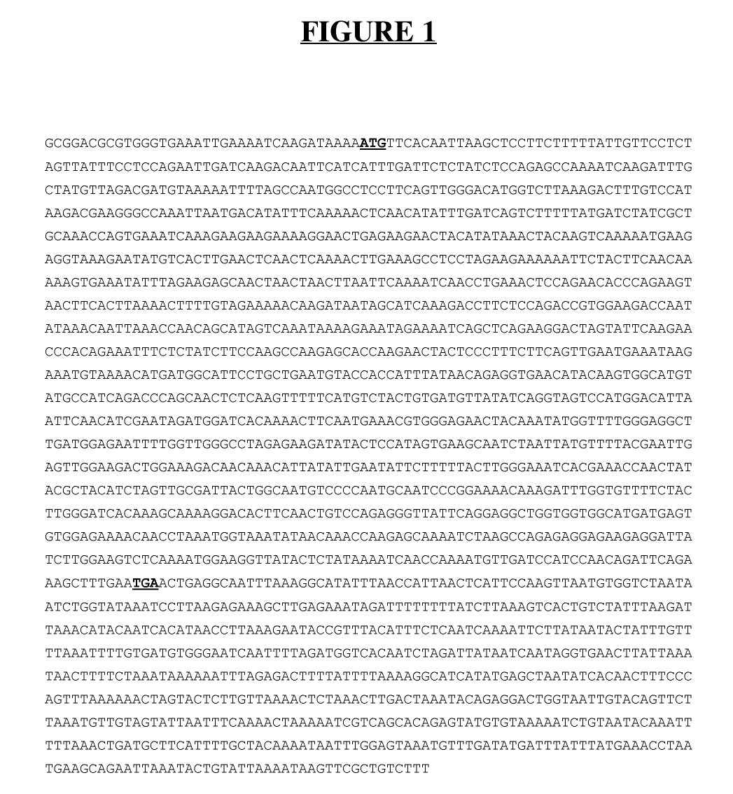

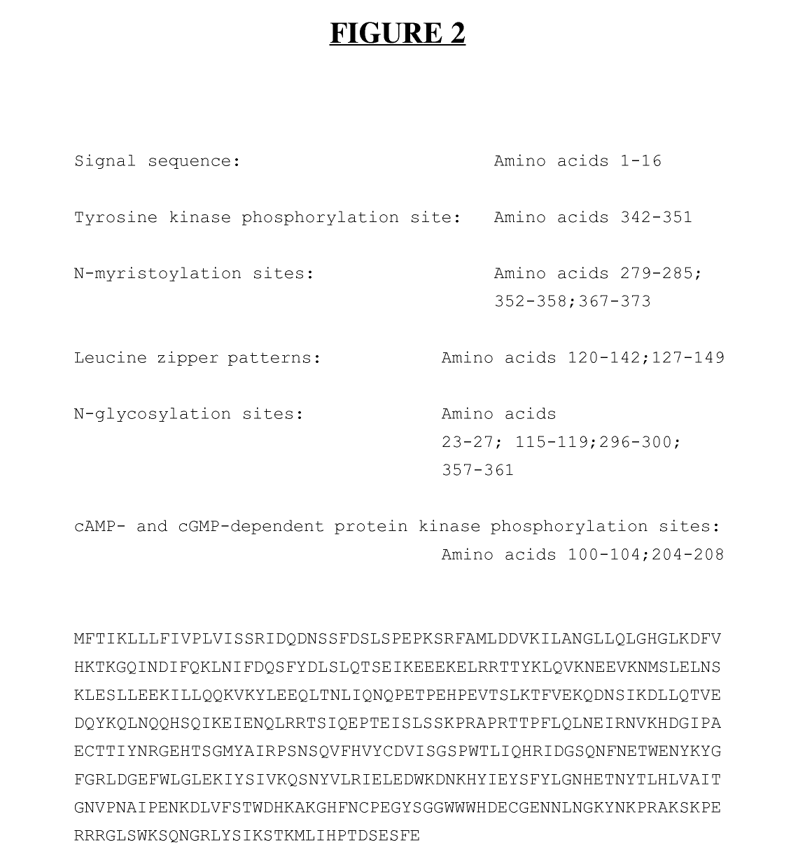

Clone DNA16451-1078 contains a single open reading frame with an apparent translational initiation site at nucleotide positions 37-39, and an apparent stop codon at nucleotide positions 1417-1419 (FIG. 1; SEQ ID NO:1). The predicted polypeptide precursor is 460 amino acids long. The full-length PRO179 protein is shown in FIG. 2 (SEQ ID NO:2).

Analysis of the full-length PRO179 sequence shown in FIG. 2 (SEQ ID NO:2) evidences the presence of important polypeptide domains as shown in FIG. 2, wherein the locations given for those important polypeptide domains are approximate as described above. Analysis of the full-length PRO179 sequence (FIG. 2; SEQ ID NO:2) evidences the presence of the following: a signal peptide from about amino acid 1 to about amino acid 16; N-glycosylation sites from about amino acid 23 to about amino acid 27, from about amino acid 115 to about amino acid 119, from about amino acid 296 to about amino acid 300, and from about amino acid 357 to about amino acid 361; cAMP- and cGMP-dependent protein kinase phosphorylation sites from about amino acid 100 to about amino acid 104 and from about amino acid 204 to about amino acid 208; a tyrosine kinase phosphorylation site from about amino acid 342 to about amino acid 351; N-myristoylation sites from about amino acid 279 to about amino acid 285, from about amino acid 352 to about amino acid 358, and from about amino acid 367 to about amino acid 373; and leucine zipper patterns from about amino acid 120 to about amino acid 142 and from about amino acid 127 to about amino 149.

Clone DNA16451-1078 has been deposited with ATCC on Sep. 18, 1997 and is assigned ATCC deposit no. 209281. The full-length PRO179 protein shown in FIG. 2 has an estimated molecular weight of about 53,637 daltons and a pI of about 6.61.

An analysis of the Dayhoff database (version 35.45 SwissProt 35) of the full-length sequence shown in FIG. 2 (SEQ ID NO:2), evidenced the presence of a fibrinogen-like domain exhibiting a high degree of sequence homology with the two known human ligands of the TIE-2 receptor (h-TIE-2L1 and h-TIE-2L2). The abbreviation “TIE” is an acronym which stands for “tyrosine kinase containing Ig and EGF homology domains” and was coined to designate a new family of receptor tyrosine kinases. Accordingly, PRO179 has been identified as a novel member of the TIE ligand family.

### Example 5

### Isolation of cDNA Clones Encoding Human PRO181 Polypeptides [UNQ155]

A cDNA sequence isolated in the amylase screen described in Example 2 above was found, by BLAST and FastA sequence alignment, to have sequence homology to a nucleotide sequence encoding the cornichon protein. This cDNA sequence is herein designated DNA13242. Based on the sequence homology, oligonucleotide probes were generated from the sequence of the DNA13242 molecule and used to screen a human placenta (LIB89) library prepared as described in paragraph 1 of Example 2 above. The cloning vector was pRK5B (pRK5B is a precursor of pRK5D that does not contain the SfiI site; see, Holmes et al., Science, 253:1278-1280 (1991)), and the cDNA size cut was less than 2800 bp.

The oligonucleotide probes employed included:

forward PCR primer
(SEQ ID NO: 156)
5′-GTGCAGCAGAGTGGCTTACA-3′
reverse PCR primer
(SEQ ID NO: 157)
5′-ACTGGACCAATTCTTCTGTG-3′
hybridization probe
(SEQ ID NO: 158)
5′-GATATTCTAGCATATTGTCAGAAGGAAGGATGGTGCAAATTAGCT-
3′

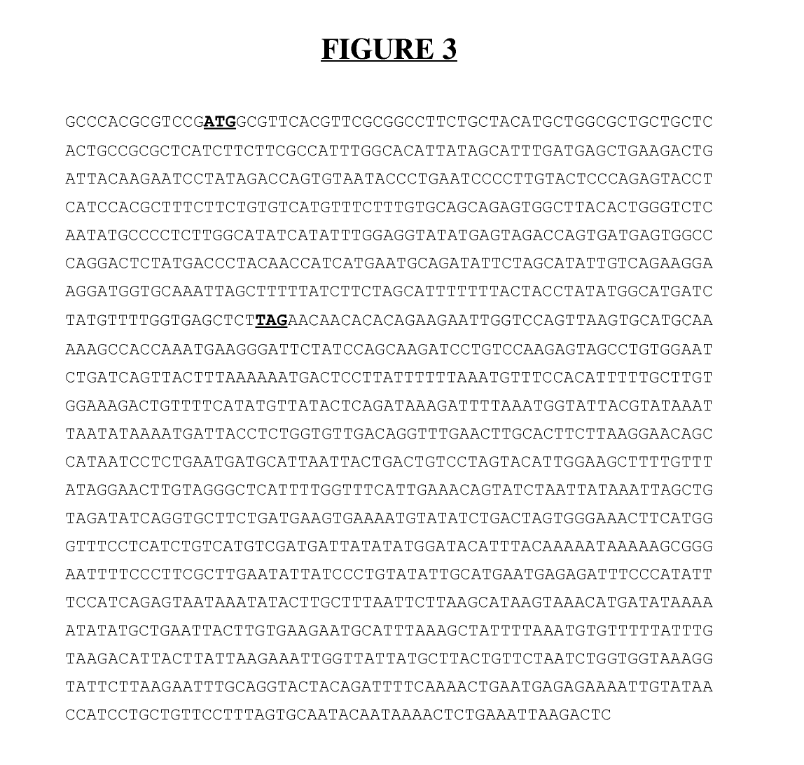

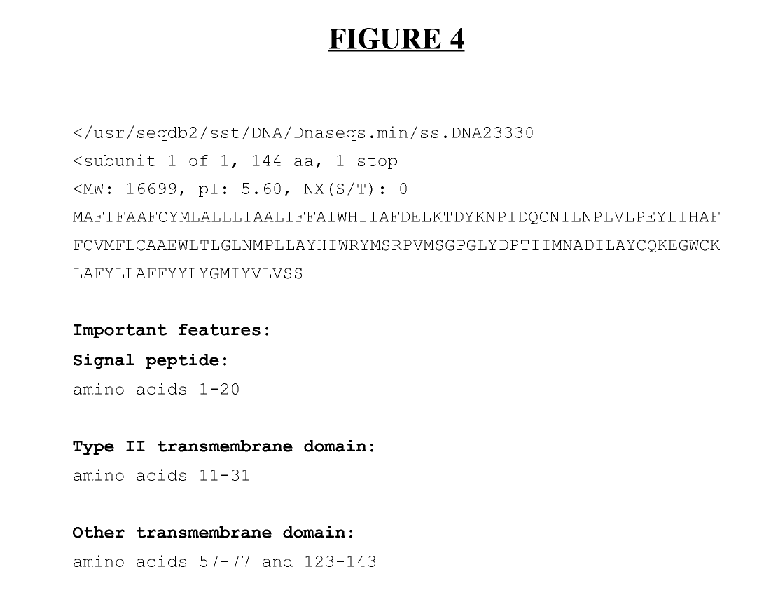

A full length clone was identified that contained a single open reading frame with an apparent translational initiation site at nucleotide positions 14-16 and ending at the stop codon found at nucleotide positions 446-448 (FIG. 3; SEQ ID NO:3). The predicted polypeptide precursor is 144 amino acids long, has a calculated molecular weight of approximately 16,699 daltons and an estimated pI of approximately 5.6. Analysis of the full-length PRO181 sequence shown in FIG. 4 (SEQ ID NO:4) evidences the presence of the following: a signal peptide from about amino acid 1 to about amino acid 20, a putative type II transmembrane domain from about amino acid 11 to about amino acid 31 and other transmembrane domains from about amino acid 57 to about amino acid 77 and from about amino acid 123 to about amino acid 143. Clone UNQ155 (DNA23330-1390) has been deposited with ATCC on Apr. 14, 1998 and is assigned ATCC deposit no. 209775.

Analysis of the amino acid sequence of the full-length PRO181 polypeptide suggests that it possesses significant sequence similarity to the cornichon protein, thereby indicating that PRO181 may be a novel cornichon homolog. More specifically, an analysis of the Dayhoff database (version 35.45 SwissProt 35) evidenced significant homology between the PRO181 amino acid sequence and the following Dayhoff sequences, AF022811_{—}1, CETO9E8_{—}3, 564058, YGF4_YEAST, YB60_YEAST, EBU89455_{—}1, SIU36383_{—}3 and PH1371.

### Example 6

### Isolation of cDNA Clones Encoding Human PRO244 Polypeptides [UNQ218]

A consensus DNA sequence was assembled relative to other EST sequences using phrap as described in Example 1 above. Based on this consensus sequence, oligonucleotides were synthesized to identify by PCR a cDNA library that contained the sequence of interest and for use as probes to isolate a clone of the full-length coding sequence for PRO244.

A pair of PCR primers (forward and reverse) were synthesized:

(SEQ ID NO: 159)
5′-TTCAGCTTCTGGGATGTAGGG-3′ (30923.f1)
(SEQ ID NO: 160)
5′-TATTCCTACCATTTCACAAATCCG-3′ (30923.r1)

A probe was also synthesized:

| | |
|---|---|
| 5′-GGAGGACTGTGCCACCATGAGAGACTCTTCAAACCCAAGGCAAAATTGG-3′ (30923.p1) | (SEQ ID NO: 161) |

In order to screen several libraries for a source of a full-length clone, DNA from the libraries was screened by PCR amplification with the PCR primer pair identified above. A positive library was then used to isolate clones encoding the PRO244 gene using the probe oligonucleotide and one of the PCR primers.

RNA for construction of the cDNA libraries was isolated from a human fetal kidney library. DNA sequencing of the clones isolated as described above gave the full-length DNA sequence and the derived protein sequence for PRO244.

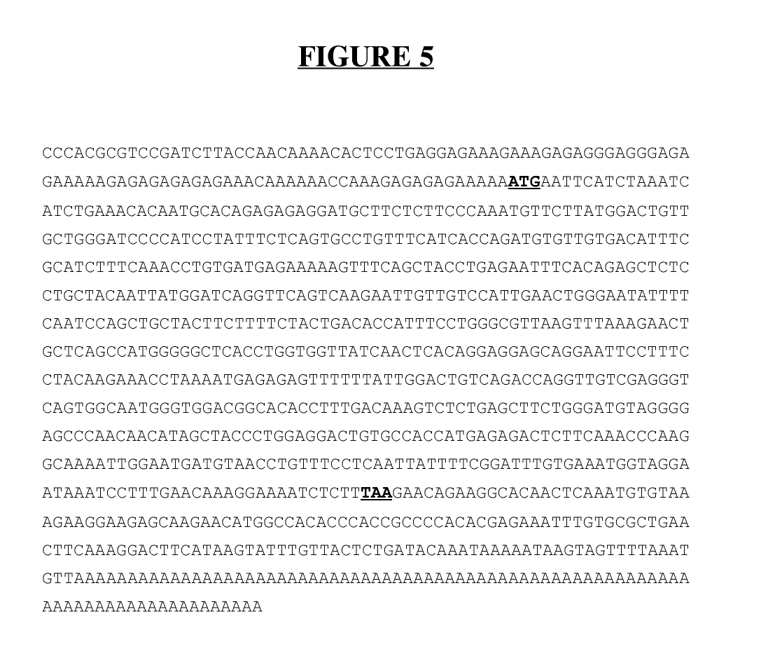

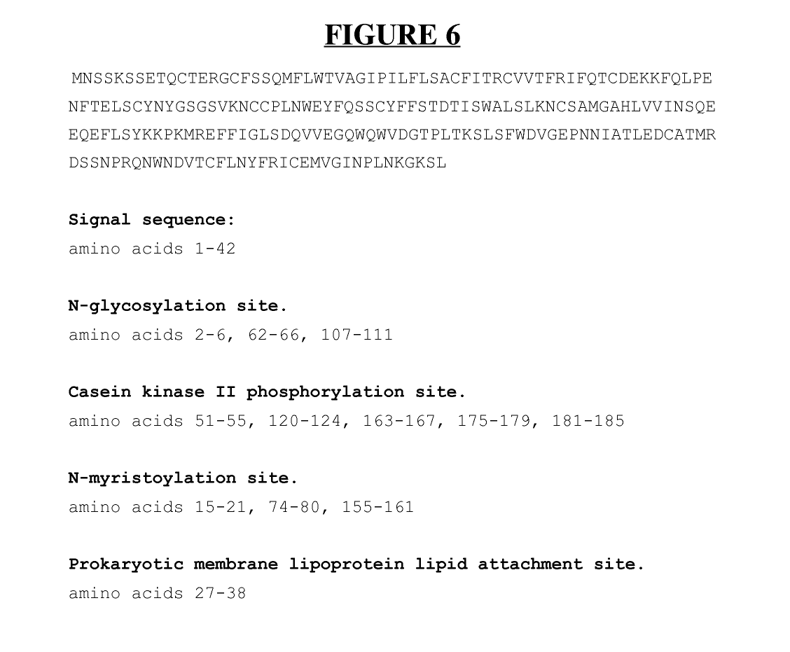

The entire nucleotide sequence of PRO244 is shown in FIG. 5 (SEQ ID NO:5). Clone DNA35668-1171 contains a single open reading frame with an apparent translational initiation site at nucleotide positions 106-108 (FIG. 5). The predicted polypeptide precursor is 219 amino acids long (FIG. 6; SEQ ID NO:6). Clone DNA35668-1171 has been deposited with ATCC on Oct. 16, 1997 (designated as DNA35668-1171) and is assigned ATCC deposit no. ATCC209371. The protein has a cytoplasmic domain (aa 1-20), a transmembrane domain (aa 21-46), and an extracellular domain (aa 47-219), with a C-lectin domain at aa 55-206.

Based on a BLAST and FastA sequence alignment analysis of the full-length sequence, PRO244 shows notable amino acid sequence identity to hepatic lectin gallus gallus (43%), HIC hp 120-binding C-type lectin (42%), macrophage lectin 2 (HUMHML2-1, 41%), and sequence PR32188 (44%).

### Example 7

### Isolation of cDNA Clones Encoding Human PRO247 Polypeptides [UNQ_{221]}

A consensus DNA sequence was assembled relative to other EST sequences using phrap as described in Example 1 above. This consensus sequence is herein designated DNA33480. Based on the DNA33480 consensus sequence, oligonucleotides were synthesized: 1) to identify by PCR a cDNA library that contained the sequence of interest, and 2) for use as probes to isolate a clone of the full-length coding sequence for PRO247.

A pair of PCR primers (forward and reverse) were synthesized:

forward PCR primer

| | |
|---|---|
| 5′-CAACAATGAGGGCACCAAGC-3′ | (SEQ ID NO: 162) |
| reverse PCR primer | |
| 5′-GATGGCTAGGTTCTGGAGGTTCTG-3′ | (SEQ ID NO: 163) |

Additionally, a synthetic oligonucleotide hybridization probe was constructed from the DNA33480 expression sequence tag which had the following nucleotide sequence

| hybridization probe | |
|---|---|
| 5′-CAACCTGCAGGAGATTGACCTCAAGGACAACAACCTCAAGACCATCG-3′ | (SEQ ID NO: 164) |

In order to screen several libraries for a source of a full-length clone, DNA from the libraries was screened by PCR amplification with the PCR primer pair identified above. A positive library was then used to isolate clones encoding the PRO247 gene using the probe oligonucleotide and one of the PCR primers.

RNA for construction of the cDNA libraries was isolated from human fetal brain tissue.

DNA sequencing of the clones isolated as described above gave the full-length DNA sequence for PRO247 [herein designated as DNA35673-1201] (SEQ ID NO:7) and the derived protein sequence for PRO247.

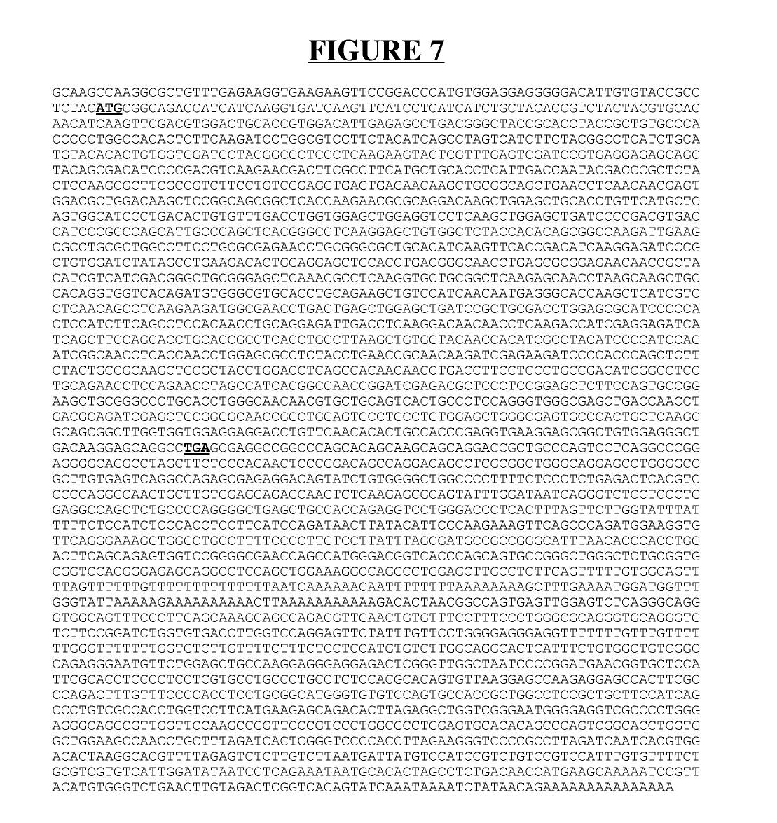

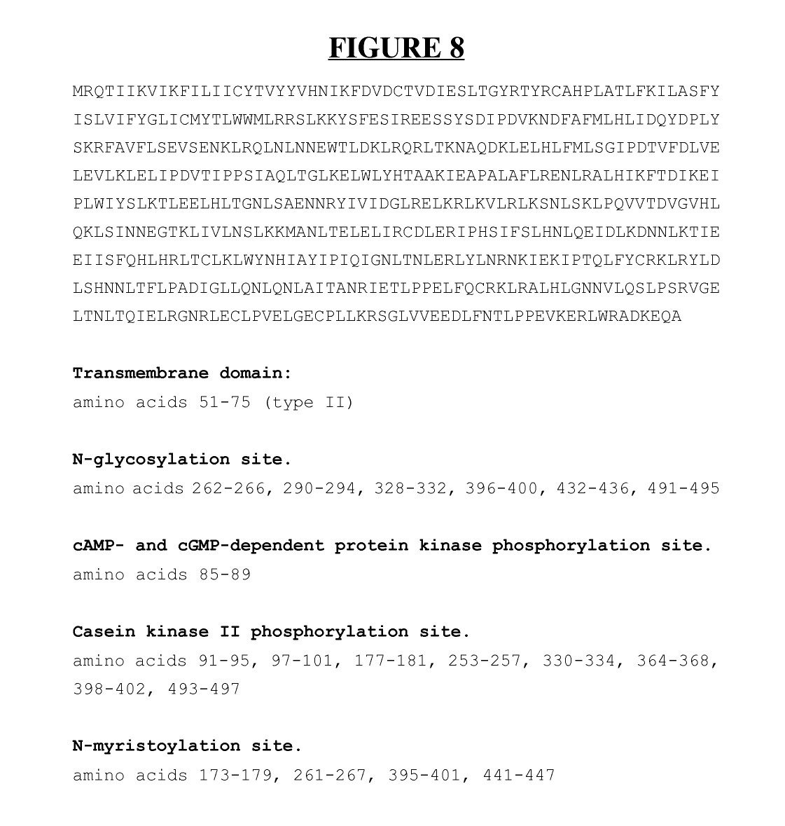

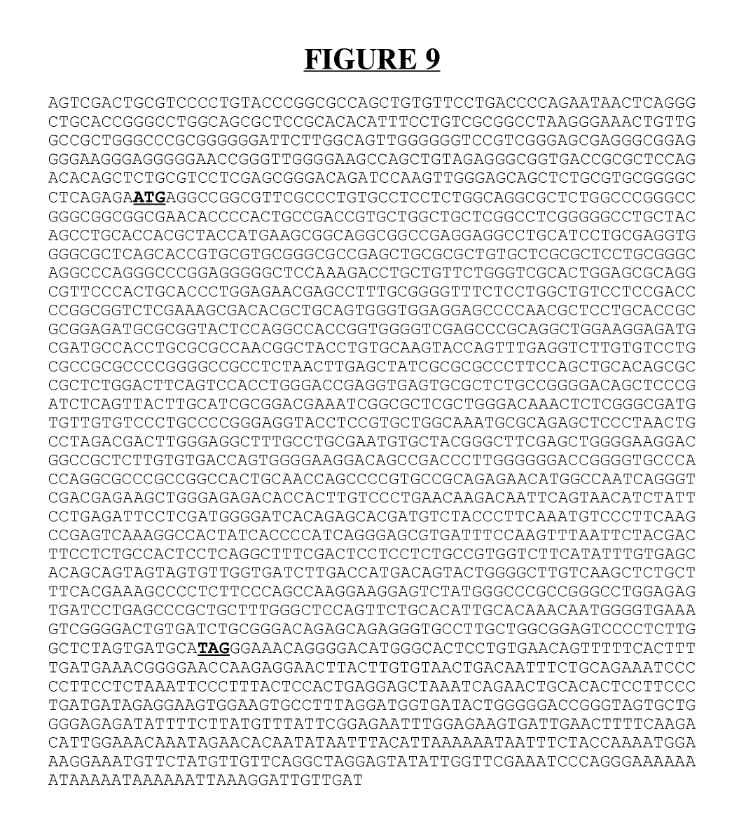

The entire nucleotide sequence of DNA35673-1201 is shown in FIG. 7 (SEQ ID NO:7). Clone DNA35673-1201 contains a single open reading frame with an apparent translational initiation site at nucleotide positions 80-82 of SEQ ID NO:249 and ending at the stop codon after nucleotide position 1717 of SEQ ID NO:7 (FIG. 7). The predicted polypeptide precursor is 546 amino acids long (FIG. 8; SEQ ID NO:8). Clone DNA35673-1201 has been deposited with ATCC on Oct. 28, 1997 and is assigned ATCC deposit no. 209418.

Analysis of the amino acid sequence of the full-length PRO247 polypeptide suggests that portions of it possess significant homology to the densin molecule and KIAA0231, thereby indicating that PRO247 may be a novel leucine rich repeat protein.

### Example 8

### Isolation of cDNA Clones Encoding Human PRO269 Polypeptides [UNQ_{236]}

A consensus DNA sequence was assembled relative to other EST sequences using phrap as described in Example 1 above. This consensus sequence is herein designated DNA35705. Based on the DNA35705 consensus sequence, oligonucleotides were synthesized: 1) to identify by PCR a cDNA library that contained the sequence of interest, and 2) for use as probes to isolate a clone of the full-length coding sequence for PRO269.

Forward and reverse PCR primers were synthesized:

forward PCR primer (.f1)

| | |
|---|---|
| 5′-TGGAAGGAGATGCGATGCCACCTG-3′ | (SEQ ID NO: 165) |
| forward PCR primer (.f2) | |
| 5′-TGACCAGTGGGGAAGGACAG-3′ | (SEQ ID NO: 166) |
| forward PCR primer (.f3) | |
| 5′-ACAGAGCAGAGGGTGCCTTG-3′ | (SEQ ID NO: 167) |
| reverse PCR primer (.r1) | |
| 5′-TCAGGGACAAGTGGTGTCTCTCCC-3′ | (SEQ ID NO: 168) |

reverse PCR primer(.r2)

| | |
|---|---|
| 5′-TCAGGGAAGGAGTGTGCAGTTCTG-3′ | (SEQ ID NO: 169) |

Additionally, a synthetic oligonucleotide hybridization probe was constructed from the consensus DNA35705 sequence which had the following nucleotide sequence:

| hybridization probe | |
|---|---|
| 5′-ACAGCTCCCGATCTCAGTTACTTGCATCGCGGACGAAATCGGCGCTCGCT-3′ | (SEQ ID NO: 170) |

In order to screen several libraries for a source of a full-length clone, DNA from the libraries was screened by PCR amplification with the PCR primer pairs identified above. A positive library was then used to isolate clones encoding the PRO269 gene using the probe oligonucleotide and one of the PCR primers.

RNA for construction of the cDNA libraries was isolated from human fetal kidney tissue.

DNA sequencing of the clones isolated as described above gave the full-length DNA sequence for PRO269 [herein designated as DNA38260-1180] (SEQ ID NO:9) and the derived protein sequence for PRO269.

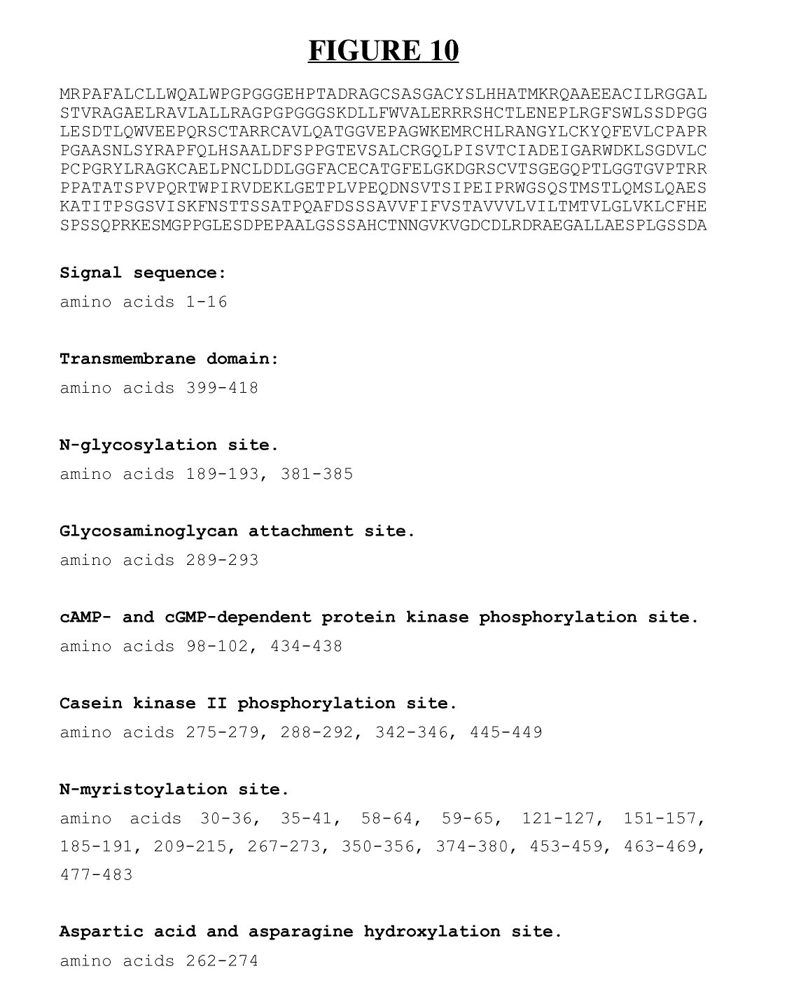

The entire nucleotide sequence of DNA38260-1180 is shown in FIG. 9 (SEQ ID NO:9). Clone DNA38260-1180 contains a single open reading frame with an apparent translational initiation site at nucleotide positions 314-316 and ending at the stop codon at nucleotide positions 1784-1786 (FIG. 9; SEQ ID NO:9). The predicted polypeptide precursor is 490 amino acids long (FIG. 10; SEQ ID NO:10). Clone DNA38260-1180 has been deposited with ATCC on Oct. 17, 1997 and is assigned ATCC deposit no. ATCC 209397.

Analysis of the amino acid sequence of the full-length PRO269 suggests that portions of it possess significant homology to the human thrombomodulin proteins, thereby indicating that PRO269 may possess one or more thrombomodulin-like domains.

### Example 9

### Isolation of cDNA Clones Encoding Human PRO293 Polypeptides [UNQ256]

The extracellular domain (ECD) sequences (including the secretion signal, if any) of from about 950 known secreted proteins from the Swiss-Prot public protein database were used to search expressed sequence tag (EST) databases. The EST databases included public EST databases (e.g., GenBank) and a proprietary EST DNA database (LIFESEQ®, Incyte Pharmaceuticals, Palo Alto, Calif.). The search was performed using the computer program BLAST or BLAST2 (Altshul et al., Methods in Enzymology 266:460-480 (1996)) as a comparison of the ECD protein sequences to a 6 frame translation of the EST sequence. Those comparisons resulting in a BLAST score of 70 (or in some cases 90) or greater that did not encode known proteins were clustered and assembled into consensus DNA sequences with the program “pinup” (Phil Green, University of Washington, Seattle, Wash.).

Based on an expression tag sequence designated herein as T08294 identified in the above analysis, oligonucleotides were synthesized: 1) to identify by PCR a cDNA library that contained the sequence of interest, and 2) for use as probes to isolate a clone of the full-length coding sequence for PRO293.

A pair of PCR primers (forward and reverse) were synthesized:

forward PCR primer

| | |
|---|---|
| 5′-AACAAGGTAAGATGCCATCCTG-3′ | (SEQ ID NO: 171) |
| reverse PCR primer | |
| 5′-AAACTTGTCGATGGAGACCAGCTC-3′ | (SEQ ID NO: 172) |

Additionally, a synthetic oligonucleotide hybridization probe was constructed from the expression sequence tag which had the following nucleotide sequence

hybridization probe
(SEQ ID NO: 173)
5′-AGGGGCTGCAAAGCCTGGAGAGCCTCTCCTTCTATGACAACCAGC-
3′

In order to screen several libraries for a source of a full-length clone, DNA from the libraries was screened by PCR amplification with the PCR primer pair identified above. A positive library was then used to isolate clones encoding the PRO293 gene using the probe oligonucleotide and one of the PCR primers.

RNA for construction of the cDNA libraries was isolated from human fetal brain tissue.

DNA sequencing of the clones isolated as described above gave the full-length DNA sequence for PRO293 [herein designated as DNA37151-1193] (SEQ ID NO:11) and the derived protein sequence for PRO293.

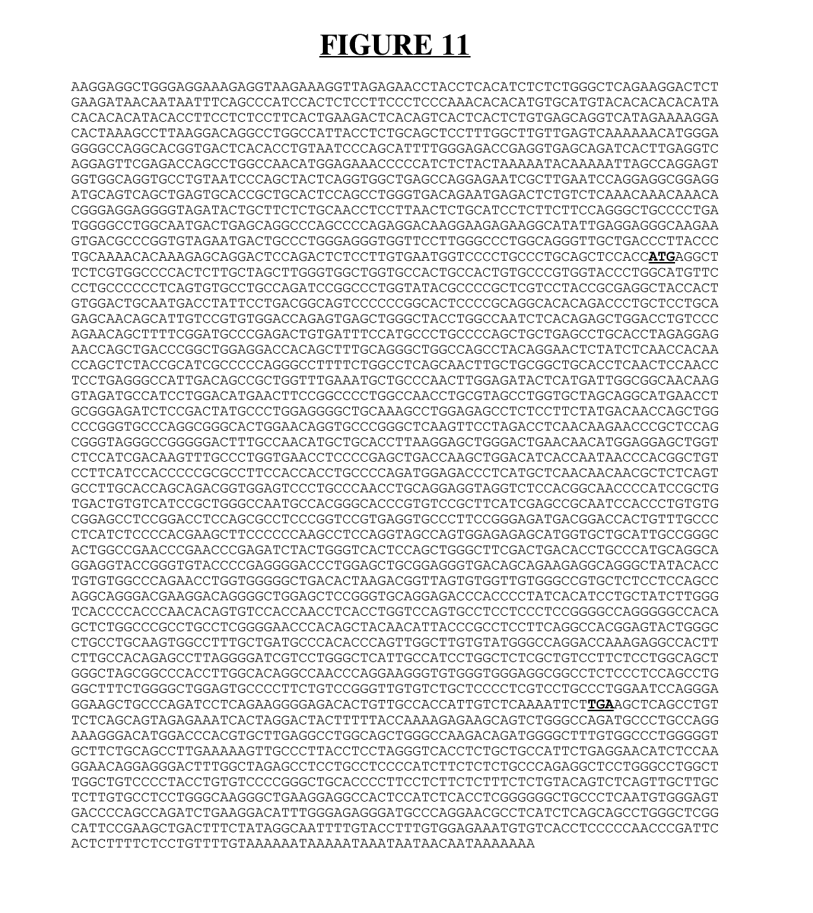

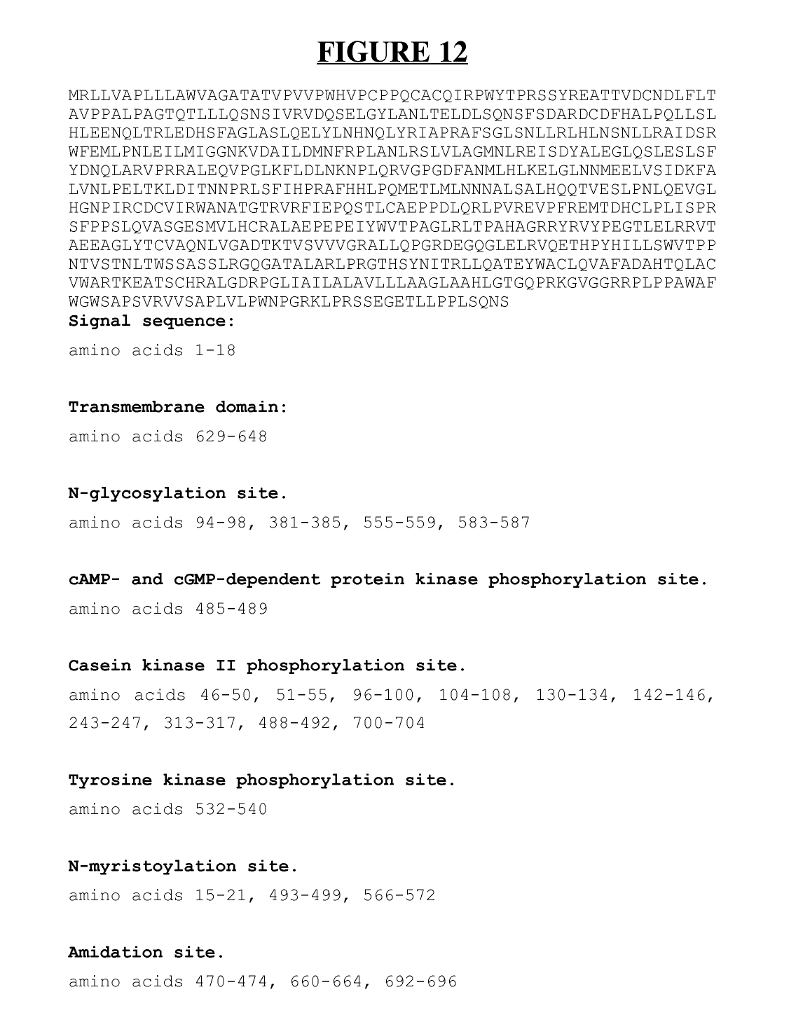

The entire nucleotide sequence of DNA37151-1193 is shown in FIG. 11 (SEQ ID NO:11). Clone DNA37151-1193 contains a single open reading frame with an apparent translational initiation site at nucleotide positions 881-883 and ending at the stop codon after nucleotide position 3019 of SEQ ID NO:11, FIG. 11). The predicted polypeptide precursor is 713 amino acids long (FIG. 12; SEQ ID NO:12). Clone DNA37151-1193 has been deposited with ATCC on Oct. 17, 1997 and is assigned ATCC deposit no. ATCC 209393.

Analysis of the amino acid sequence of the full-length PRO293 polypeptide suggests that portions of it possess significant homology to the NLRR proteins, thereby indicating that PRO293 may be a novel NLRR protein.

### Example 10

### Isolation of cDNA Clones Encoding Human PRO298 Polypeptides [UNQ261]

A cDNA isolated in the amylase screen described in Example 2 above is herein designated DNA26832. The sequence of DNA26832 was then used to search expressed sequence tag (EST) databases. The EST databases included public EST databases (e.g., GenBank) and a proprietary EST database (LIFESEQ®, Incyte Pharmaceuticals, Palo Alto, Calif.). The search was performed using the computer program BLAST or BLAST2 (Altshul et al., Methods in Enzymology 266: 469-480 [1996]). Those comparisons resulting in a BLAST score of 70 (or in some cases 90) or greater that did not encode proteins were clustered and assembled into consensus DNA sequences with the program “phrap” (Phil Green, University of Washington, Seattle, Wash.).

A consensus DNA sequence was assembled relative to other EST sequences using phrap. A consensus sequence was determined, which was then extended using repeated cycles of BLAST and phrap to extend the consensus sequence as far as possible using the sources of EST sequences discussed above. The extended assembly sequence was designated DNA35861. Based on the DNA35861 consensus sequence, oligonucleotides were synthesized: 1) to identify by PCR a cDNA library that contained the sequence of interest, and 2) for use as probes to isolate a clone of the full-length coding sequence of PRO298. Forward and reverse primers generally range from 20 to 30 nucleotides and are often designed to give a PCR product of about 100-1000 by in length. The probe sequence is typically 40-55 by in length. In some cases, additional oligonucleotides are synthesized when the consensus sequence is greater than about 1-1.5 kbp. In order to screen several libraries for a full-length clone, DNA from the libraries was screened by PCR amplification, as per Ausubel et al., Current Protocols in Molecular Biology, with the PCR primer pair. A positive library was used to isolate clones encoding the gene of interest using the probe oligonucleotide and one of the primer pairs.

PCR primers (forward and reverse) and a hybridization probe were synthesized:

forward PCR primer 1
(SEQ ID NO: 174)
CAACGTGATTTCAAAGCTGGGCTC
forward PCR primer 2
(SEQ ID NO: 175)
GCCTCGTATCAAGAATTTCC
forward PCR primer 3
(SEQ ID NO: 176)
AGTGGAAGTCGACCTCCC
reverse PCR primer 1
(SEQ ID NO: 177)
CTCACCTGAAATCTCTCATAGCCC
hybridization probe 1
(SEQ ID NO: 178)
CGCAAAACCCATTTTGGGAGCAGGAATTCCAATCATGTCTGTGATGGTGG

In order to screen several libraries for a source of a full-length clone, DNA from the libraries was screened by PCR amplification with the PCR primer pair identified above. A positive library was then used to isolate clones encoding the PRO298 gene using the probe oligonucleotide and one of the PCR primers.

RNA for construction of the cDNA libraries was isolated from human fetal lung tissue (LIB25). The cDNA libraries used to isolated the cDNA clones were constructed by standard methods using commercially available reagents such as those from Invitrogen, San Diego, Calif. The cDNA was primed with oligo dT containing a NotI site, linked with blunt to SalI hemikinased adaptors, cleaved with NotI, sized appropriately by gel electrophoresis, and cloned in a defined orientation into a suitable cloning vector (such as pRKB or pRKD; pRK5B is a precursor of pRK5D that does not contain the SfiI site; see, Holmes et al., Science, 253:1278-1280 (1991)) in the unique XhoI and NotI sites.

DNA sequencing of the clones isolated as described above gave the full-length DNA sequence for PRO298 (herein designated UNQ261 [DNA39975-1210]) (SEQ ID NO:13), and the derived protein sequence for PRO298 (SEQ ID NO:14).

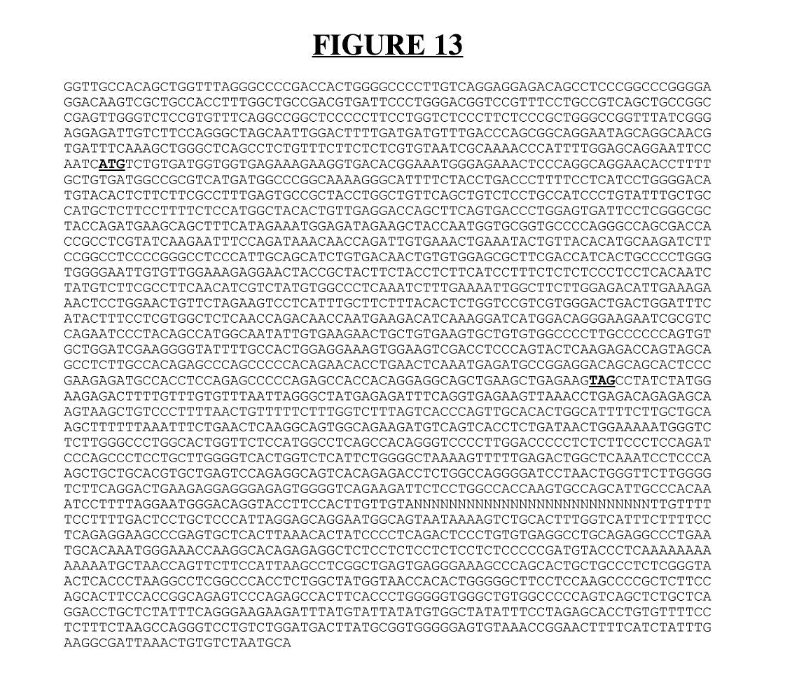

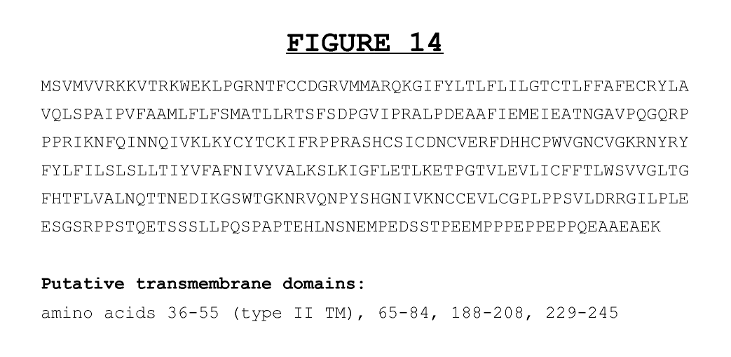

The entire nucleotide sequence of UNQ261 (DNA39975-1210) is shown in FIG. 13 (SEQ ID NO:13). Clone DNA39975-1210 contains a single open reading frame with an apparent translational initiation site at nucleotide positions 375-377. The predicted polypeptide precursor is 364 amino acids long (FIG. 14; SEQ ID NO:14). The protein contains four putative transmembrane domains between amino acid positions 36-55 (type II TM), 65-84, 188-208, and 229-245, respectively. A putative N-linked glycosylation site starts at amino acid position 253. In addition, the following features have been identified in the protein sequence: cAMP- and cGMP-dependent protein kinase phosphorylation site, starting at position 8; N-myristoylation sites starting a position 173 and 262, respectively; and a ZP domain between amino acid positions 45-60. Clone DNA39975-1210 has been deposited with ATCC (Apr. 21, 1998) and is assigned ATCC deposit no. 209783.

### Example 11

### Isolation of cDNA Clones Encoding Human PRO339 Polypeptides [UNO299]

An expressed sequence tag (EST) DNA database (LIFESEQ®, Incyte Pharmaceuticals, Palo Alto, Calif.) was searched and ESTs were identified. An assembly of Incyte clones and a consensus sequence was formed using phrap as described in Example 1 above.

Forward and reverse PCR primers were synthesized based upon the assembly-created consensus sequence:

forward PCR primer 1

| | |
|---|---|
| 5′-GGGATGCAGGTGGTGTCTCATGGGG-3′ | (SEQ ID NO: 179) |
| forward PCR primer 2 | |
| 5′-CCCTCATGTACCGGCTCC-3′ | (SEQ ID NO: 180) |
| forward PCR primer 3 | |
| 5′-GTGTGACACAGCGTGGGC-3′ | (SEQ ID NO: 181) |
| forward PCR primer 4 | |
| 5′-GACCGGCAGGCTTCTGCG-3′ | (SEQ ID NO: 182) |
| reverse PCR primer 1 | |
| 5′-CAGCAGCTTCAGCCACCAGGAGTGG-3′ | (SEQ ID NO: 183) |
| reverse PCR primer 2 | |
| 5′-CTGAGCCGTGGGCTGCAGTCTCGC-3′ | (SEQ ID NO: 184) |

Additionally, a synthetic oligonucleotide hybridization probe was constructed from the consensus sequence which had the following nucleotide sequence

hybridization probe
(SEQ ID NO: 185)
5′-CCGACTACGACTGGTTCTTCATCATGCAGGATGACACATATGTGC-
3'

In order to screen several libraries for a source of a full-length clone, DNA from the libraries was screened by PCR amplification with the PCR primer pairs identified above. A positive library was then used to isolate clones encoding the PRO339 gene using the probe oligonucleotide and one of the PCR primers.

RNA for construction of the cDNA libraries was isolated from human fetal liver tissue.

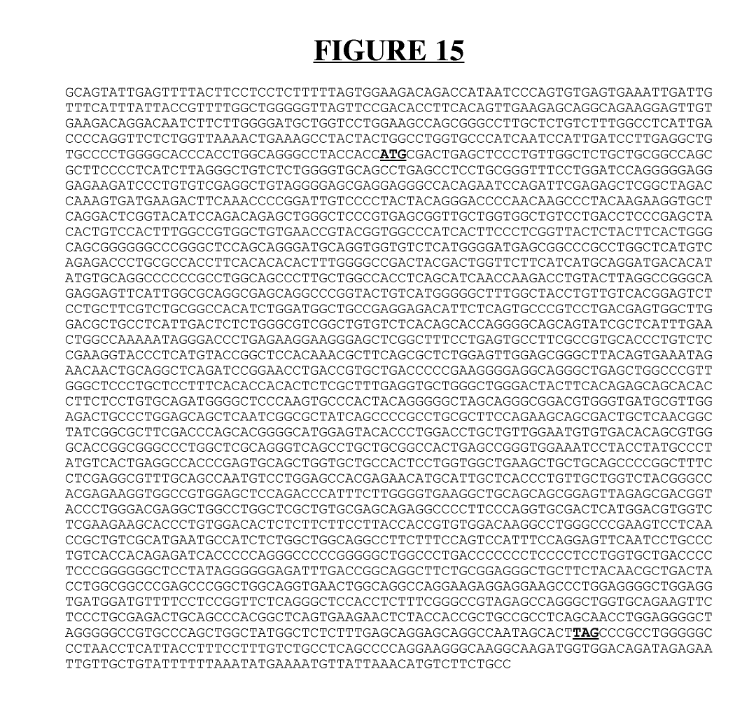

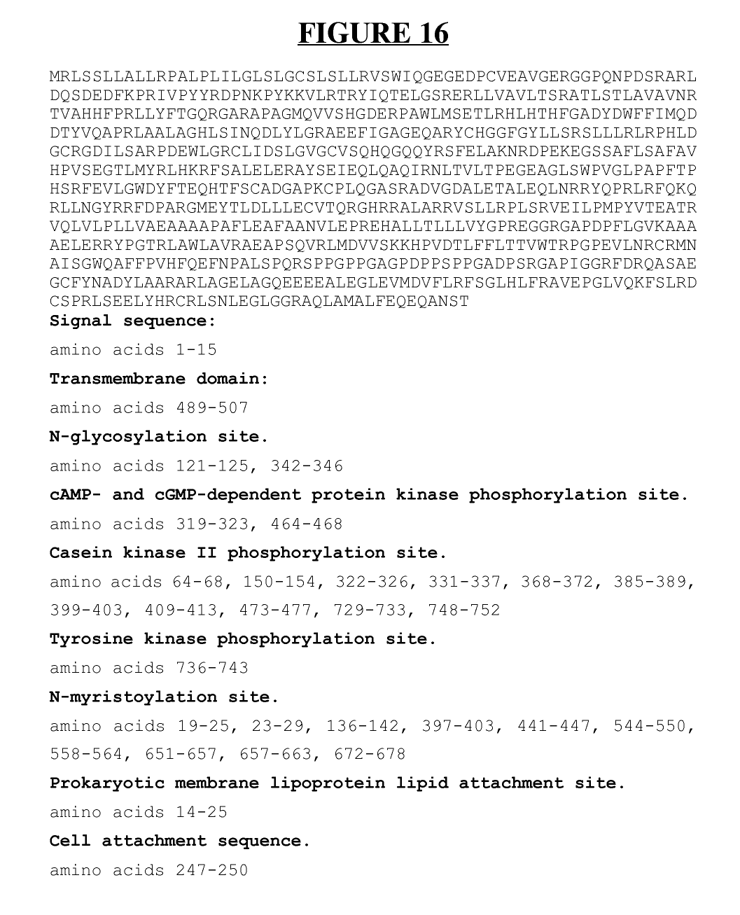

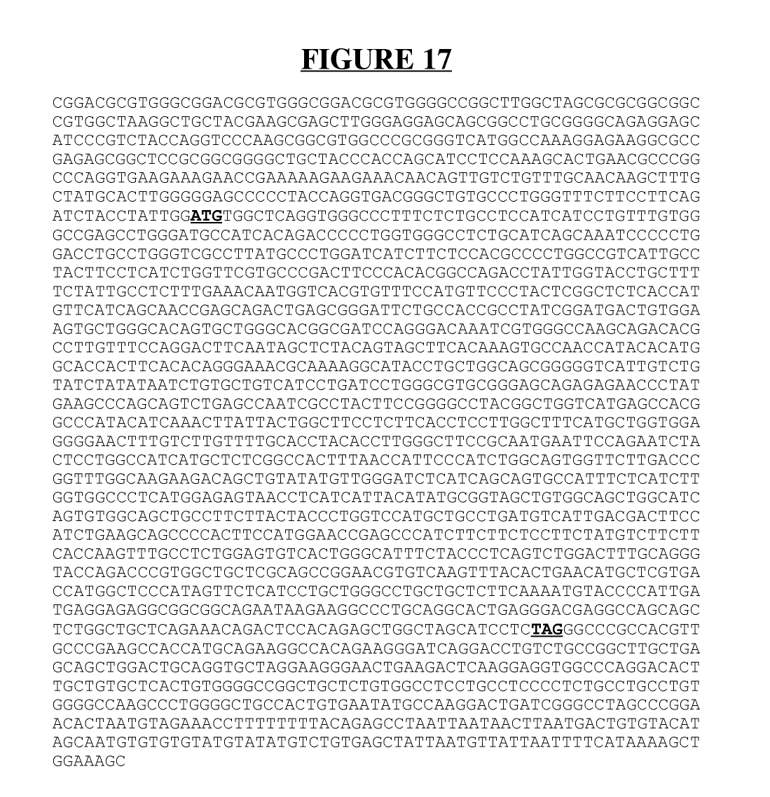

A cDNA clone was sequenced in entirety. The entire nucleotide sequence of DNA43466-1225 is shown in FIG. 15 (SEQ ID NO:15). Clone DNA43466-1225 contains a single open reading frame with an apparent translational initiation site at nucleotide positions 333-335 and ending at the stop codon found at nucleotide positions 2649-2651 (FIG. 15; SEQ ID NO:15). The predicted polypeptide precursor is 772 amino acids long and has a calculated molecular weight of approximately 86,226 daltons (FIG. 16; SEQ ID NO:16). Clone DNA43466-1225 has been deposited with ATCC (Nov. 21, 1997) and is assigned ATCC deposit no. ATCC 209490.

Based on a BLAST and FastA sequence alignment analysis (using the ALIGN computer program) of the full-length sequence, PRO339 has homology to C. elegans proteins and collagen-like polymer sequences as well as to fringe, thereby indicating that PRO339 may be involved in development or tissue growth.

### Example 12

### Isolation of cDNA Clones Encoding Human PRO341 Polypeptides [UNQ300]

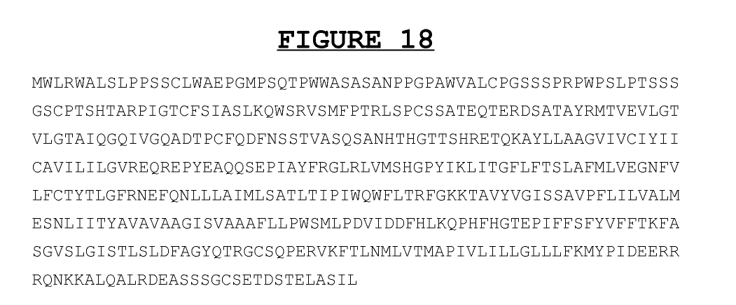

A clone designated herein as DNA12920 was isolated as described in Example 2 above from a human placenta tissue library. The DNA12920 sequence was then compared to various EST databases including public EST databases (e.g., GenBank), and a proprietary EST database (LIFESEQ®, Incyte Pharmaceuticals, Palo Alto, Calif.) to identify homologous EST sequences. The comparison was performed using the computer program BLAST or BLAST2 [Altschul et al., Methods in Enzymology, 266:460-480 (1996)]. Those comparisons resulting in a BLAST score of 70 (or in some cases, 90) or greater that did not encode known proteins were clustered and assembled into a consensus DNA sequence with the program “phrap” (Phil Green, University of Washington, Seattle, Wash.). This consensus sequence is herein designated DNA25314. Oligonucleotide primers based upon the DNA25314 sequence were then synthesized and employed to screen a human placenta cDNA library which resulted in the identification of the DNA26288-1239 clone shown in FIG. 17. The cloning vector was pRK5B (pRK5B is a precursor of pRK5D that does not contain the SfiI site; see, Holmes et al., Science, 253:1278-1280 (1991)), and the cDNA size cut was less than 2800 bp.

A full length clone was identified that contained a single open reading frame with an apparent translational initiation site at nucleotide positions 380-382, and a stop signal at nucleotide positions 1754-1756 (FIG. 17, SEQ ID NO:17). The predicted polypeptide precursor is 458 amino acids long, has a calculated molecular weight of approximately 50,264 daltons and an estimated pI of approximately 8.17. Analysis of the full-length PRO341 sequence shown in FIG. 18 (SEQ ID NO:18) evidences the presence of the following: a signal peptide from about amino acid 1 to about amino acid 17, transmembrane domains from about amino acid 171 to about amino acid 190, from about amino acid 220 to about amino acid 239, from about amino acid 259 to about amino acid 275, from about amino acid 286 to about amino acid 305, from about amino acid 316 to about amino acid 335, from about amino acid 353 to about amino acid 378 and from about amino acid 396 to about amino acid 417 and potential N-glycosylation sites from about amino acid 145 to about amino acid 147 and from about amino acid 155 to about amino acid 158. Clone DNA26288-1239 has been deposited with ATCC on Apr. 21, 1998 and is assigned ATCC deposit no. 209792.

An analysis of the Dayhoff database (version 35.45 SwissProt 35), using a WU-BLAST-2 sequence alignment analysis of the full-length sequence shown in FIG. 18 (SEQ ID NO:18), evidenced homology between the PRO341 amino acid sequence and the following Dayhoff sequences: 575696, H69788, D69852, A69888, B64918, F64752, LPU89276_{—}1, G64962, S52977 and S44253.

### Example 13

### Isolation of cDNA Clones Encoding Human PRO347 Polypeptides [UNQ306]

A consensus DNA sequence was assembled relative to other EST sequences as described in Example 1 above. This consensus sequence is herein designated DNA39499. Based on the DNA39499 consensus sequence, oligonucleotides were synthesized: 1) to identify by PCR a cDNA library that contained the sequence of interest, and 2) for use as probes to isolate a clone of the full-length coding sequence for PRO347.

PCR primers (forward and reverse) were synthesized as follows:

forward PCR primer

| | |
|---|---|
| 5′-AGGAACTTCTGGATCGGGCTCACC-3′ | (SEQ ID NO: 186) |
| reverse PCR primer | |
| 5′-GGGTCTGGGCCAGGTGGAAGAGAG-3′ | (SEQ ID NO: 187) |

Additionally, a synthetic oligonucleotide hybridization probe was constructed from the consensus DNA39499 sequence which had the following nucleotide sequence

hybridization probe
(SEQ ID NO: 188)
5′-GCCAAGGACTCCTTCCGCTGGGCCACAGGGGAGCACCAGGCCTTC-
3'

In order to screen several libraries for a source of a full-length clone, DNA from the libraries was screened by PCR amplification with the PCR primer pair identified above. A positive library was then used to isolate clones encoding the PRO347 gene using the probe oligonucleotide and one of the PCR primers. RNA for construction of the cDNA libraries was isolated from human fetal kidney tissue (LIB228).

DNA sequencing of the clones isolated as described above gave the full-length DNA sequence for PRO347 [herein designated as DNA44176-1244] (SEQ ID NO:19) and the derived protein sequence for PRO347.

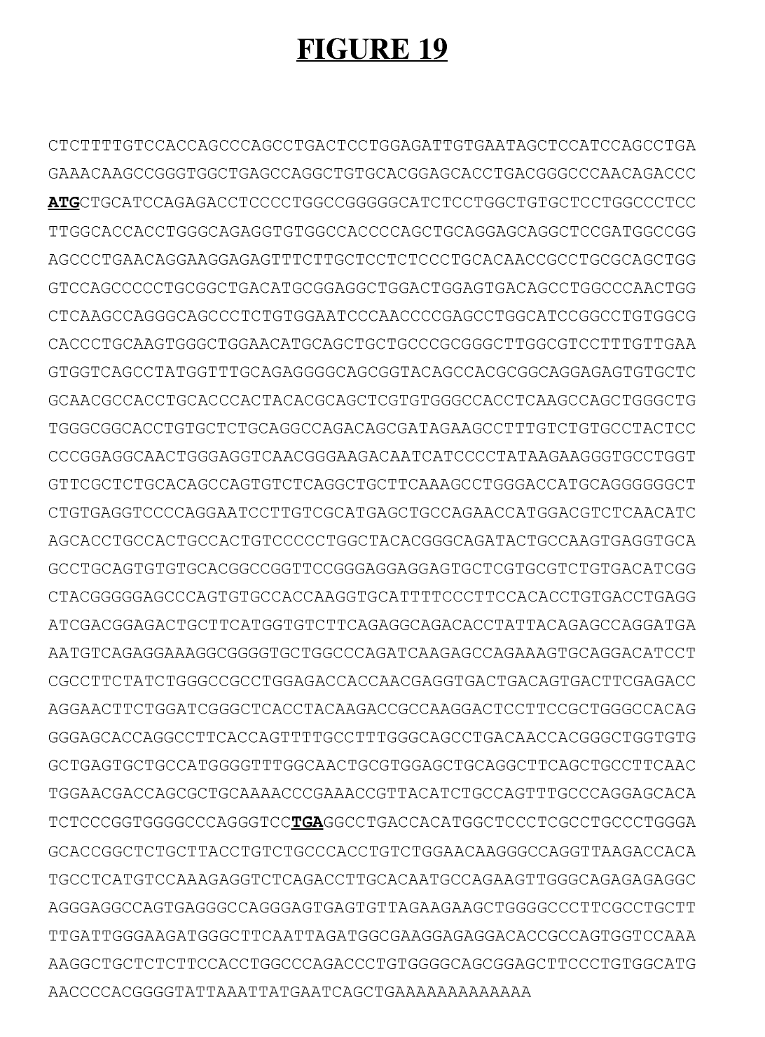

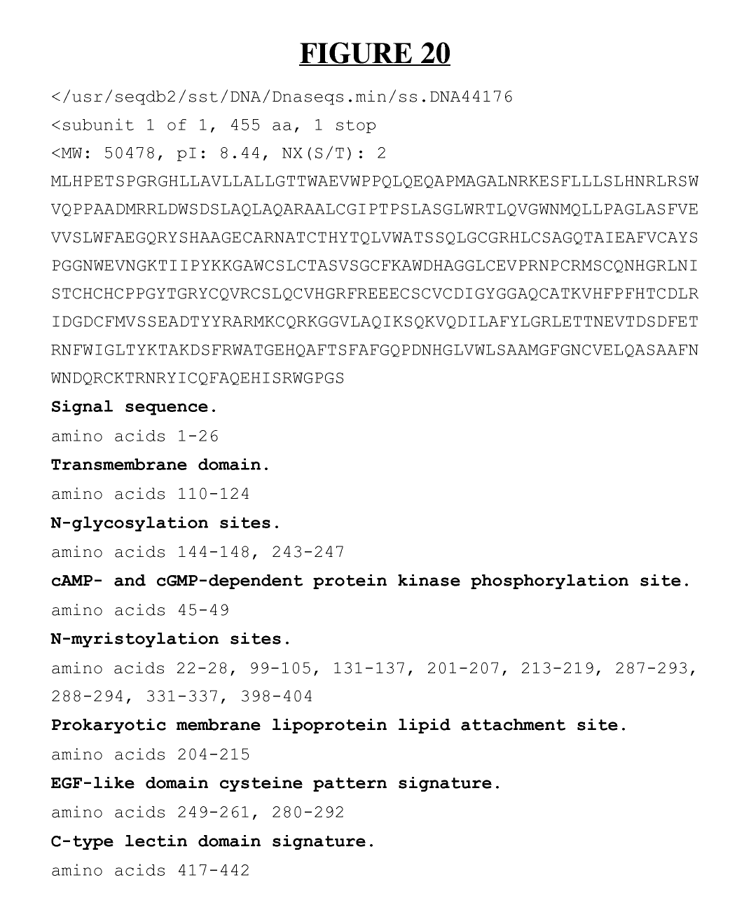

The entire nucleotide sequence of DNA44176-1244 is shown in FIG. 19 (SEQ ID NO:19). Clone DNA44176-1244 contains a single open reading frame with an apparent translational initiation site at nucleotide positions 123-125 and ending at the stop codon at nucleotide positions 1488-1490 (FIG. 19). The predicted polypeptide precursor is 455 amino acids long (FIG. 20; SEQ ID NO:20). The full-length PRO347 protein shown in FIG. 20 has an estimated molecular weight of about 50,478 daltons and a pI of about 8.44. Clone DNA44176-1244 has been deposited with ATCC (Dec. 10, 1997) and is assigned ATCC deposit no. ATCC 209532.

Analysis of the amino acid sequence of the full-length PRO347 polypeptide suggests that portions of it possess significant homology to various cysteine-rich secretory proteins, thereby indicating that PRO347 may be a novel cysteine-rich secretory protein.

### Example 14

### Isolation of cDNA Clones Encoding Human PRO531 Polypeptides [UNQ332]

An ECD database was searched and an expressed sequence tag (EST) from LIFESEQ®, Incyte Pharmaceuticals, Palo Alto, Calif. was identified which showed homology to protocadherin 3. Based on this sequence, a search was performed using the computer program BLAST or BLAST2 (Altshul et al., Methods in Enzymology 266:460-480 (1996)) as a comparison of the ECD protein sequences to a 6 frame translation of the EST sequence. Those comparisons resulting in a BLAST score of 70 (or in some cases 90) or greater that did not encode known proteins were clustered and assembled into consensus DNA sequences with the program “phrap” (Phil Green, University of Washington, Seattle, Wash.).

A consensus DNA sequence was assembled relative to other EST sequences using phrap. Based on the consensus sequence obtained, oligonucleotides were synthesized: 1) to identify by PCR a cDNA library that contained the sequence of interest, and 2) for use as probes to isolate a clone of the full-length coding sequence for PRO531.

A pair of PCR primers (forward and reverse) were synthesized:

| | |
|---|---|
| forward PCR primer | |
| 5′-CTGAGAACGCGCCTGAAACTGTG-3′; | (SEQ ID NO: 189) |
| reverse PCR primer | |
| 5′-AGCGTTGTCATTGACATCGGCG-3′. | (SEQ ID NO: 190) |

Additionally, a synthetic oligonucleotide hybridization probe was constructed from the consensus DNA sequence which had the following nucleotide sequence:

hybridization probe
(SEQ ID NO: 191)
5′-TTAGTTGCTCCATTCAGGAGGATCTACCCTTCCTCCTGAAATCCG
CGGAA-3′.

In order to screen several libraries for a source of a full-length clone, DNA from the libraries was screened by PCR amplification with the PCR primer pair identified above. A positive library was then used to isolate clones encoding the PRO531 gene using the probe oligonucleotide and one of the PCR primers. RNA for construction of the cDNA libraries was isolated from human fetal brain tissue (LIB153). The cDNA libraries used to isolate the cDNA clones were constructed by standard methods using commercially available reagents such as those from Invitrogen, San Diego, Calif. The cDNA was primed with oligo dT containing a NotI site, linked with blunt to SalI hemikinased adaptors, cleaved with NotI, sized appropriately by gel electrophoresis, and cloned in a defined orientation into a suitable cloning vector (such as pRKB or pRKD; pRK5B is a precursor of pRK5D that does not contain the SfiI site; see, Holmes et al., Science, 253:1278-1280 (1991)) in the unique XhoI and NotI sites.

DNA sequencing of the clones isolated as described above gave the full-length DNA sequence for PRO531 [herein designated as UNQ332 (DNA48314-1320)] (SEQ ID NO:21) and the derived protein sequence for PRO531.

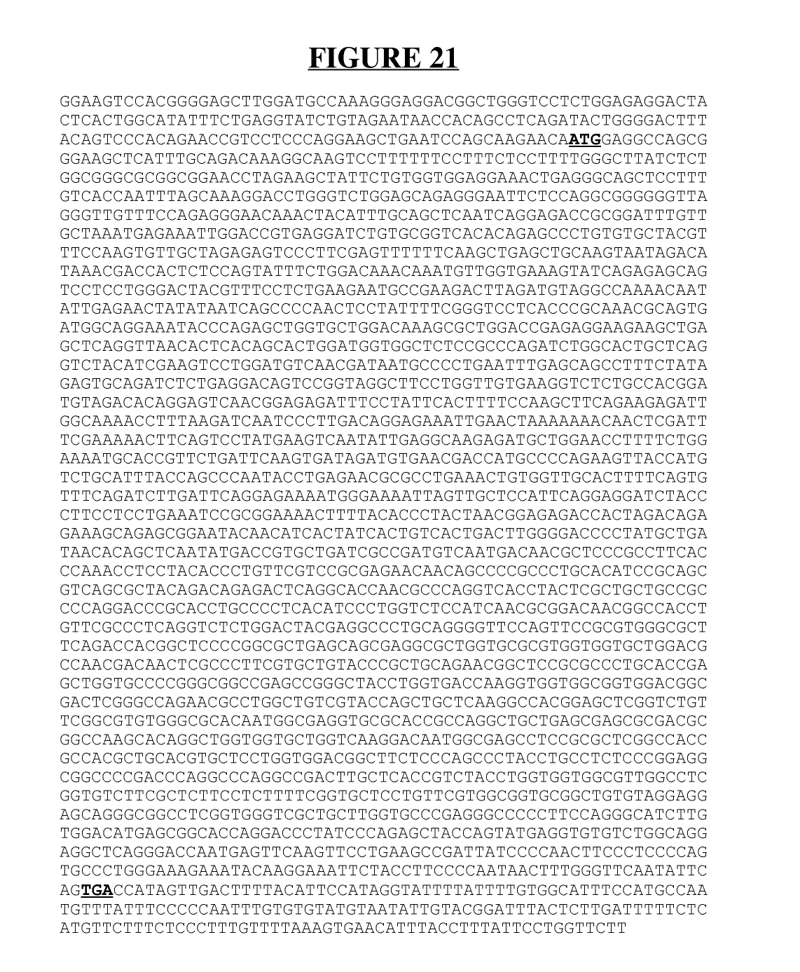

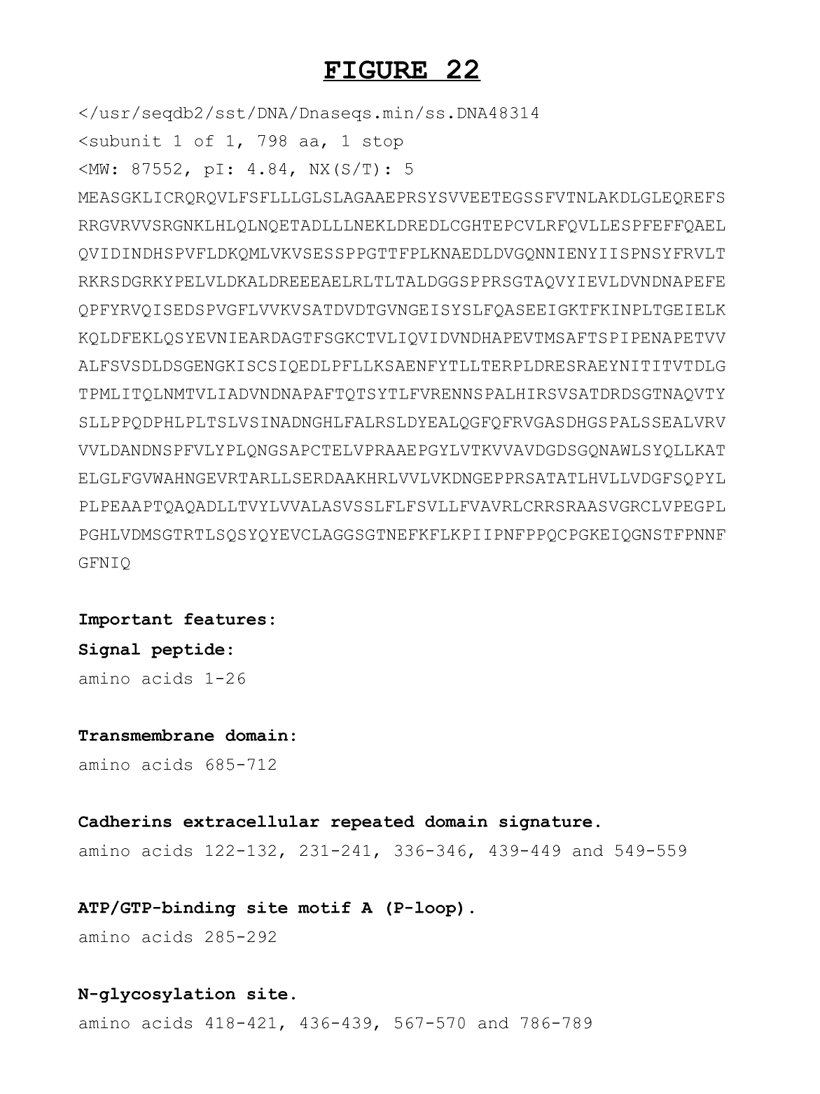

The entire representative nucleotide sequence of UNQ332 (DNA48314-1320) is shown in FIG. 21 (SEQ ID NO:21). It is understood that the actual sequence is that within the clone deposited with the ATCCas DNA48314-1320. Clone UNQ332 (DNA48314-1320) contains a single open reading frame with an apparent translational initiation site at nucleotide positions 171-173 and ending at the stop codon at nucleotide positions 2565-2567 (FIG. 21). The predicted polypeptide precursor is 789 amino acids long (FIG. 22; SEQ ID NO:22). The full-length PRO531 protein shown in FIG. 22 has an estimated molecular weight of about 87,552 daltons and a pI of about 4.84. Clone UNQ332 (DNA48314-1320) has been deposited with the ATCC on Mar. 26, 1998 as ATCC no. 209702.

Analysis of the amino acid sequence of the full-length PRO531 polypeptide suggests that portions of it possess significant homology to protocadherin 3. Moreover, PRO531 is found in the brain, like other protocadherins, thereby indicating that PRO531 is a novel member of the cadherin superfamily.

Still analyzing the amino acid sequence of SEQ ID NO:22, the cadherin extracellular repeated domain signature is found at about amino acids 122-132, 231-241, 336-346, 439-449 and 549-559 of SEQ ID NO:22. An ATP/GTP-binding site motif A (P-loop) is found at about amino acids 285-292 of SEQ ID NO:22. N-glycosylation sites are found at least at about amino acids 567-570, 786-790, 418-421 and 336-339 of SEQ ID NO:22. The signal peptide is at about amino acids 1-26, and the transmembrane domain is at about amino acids 685-712 of SEQ ID NO:22.

### Example 15

### Isolation of cDNA Clones Encoding Human PRO537 Polypeptides [UNQ338]

Use of the signal sequence algorithm described in Example 3 above allowed identification of an EST cluster sequence from the Incyte database, designated as Incyte EST cluster no. 29605. This EST cluster sequence was then compared to a variety of expressed sequence tag (EST) databases which included public EST databases (e.g., GenBank) and a proprietary EST DNA database (LIFESEQ®, Incyte Pharmaceuticals, Palo Alto, Calif.) to identify existing homologies. The homology search was performed using the computer program BLAST or BLAST2 (Altshul et al., Methods in Enzymology 266:460-480 (1996)). Those comparisons resulting in a BLAST score of 70 (or in some cases 90) or greater that did not encode known proteins were clustered and assembled into a consensus DNA sequence with the program “phrap” (Phil Green, University of Washington, Seattle, Wash.). The consensus sequence obtained therefrom is herein designated DNA48350.

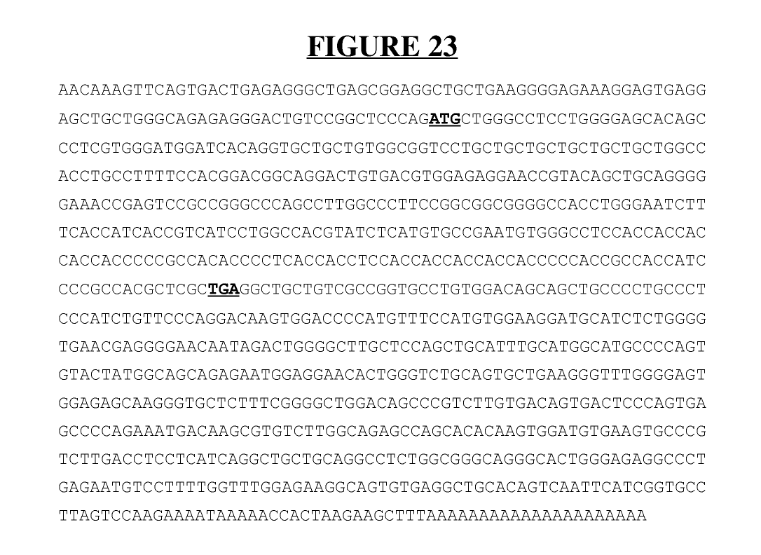

In light of an observed sequence homology between the DNA48350 consensus sequence and an EST sequence encompassed within the Merck EST clone no. R63443, the Merck EST clone R63443 was purchased and the cDNA insert was obtained and sequenced. It was found that this insert encoded a full-length protein. The sequence of this cDNA insert is shown in FIG. 23 and is herein designated as DNA49141-1431.

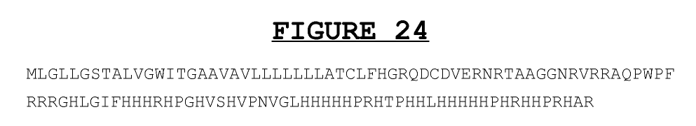

Clone DNA49141-1431 contains a single open reading frame with an apparent translational initiation site at nucleotide positions 97-99 and ending at the stop codon at nucleotide positions 442-444 (FIG. 23; SEQ ID NO:23). The predicted polypeptide precursor is 115 amino acids long (FIG. 24; SEQ ID NO:24). The full-length PRO537 protein shown in FIG. 24 has an estimated molecular weight of about 13,183 daltons and a pI of about 12.13. Analysis of the full-length PRO537 sequence shown in FIG. 24 (SEQ ID NO:24) evidences the presence of the following: a signal peptide from about amino acid 1 to about amino acid 31, a potential N-glycosylation site from about amino acid 44 to about amino acid 47, potential N-myristolation sites from about amino acid 3 to about amino acid 8 and from about amino acid 16 to about amino acid 21 and an amino acid block having homology to multicopper oxidase proteins from about amino acid 97 to about amino acid 105. Clone DNA49141-1431 has been deposited with ATCC on Jun. 23, 1998 and is assigned ATCC deposit no. 203003.

An analysis of the Dayhoff database (version 35.45 SwissProt 35), using a WU-BLAST-2 sequence alignment analysis of the full-length sequence shown in FIG. 24 (SEQ ID NO:24), evidenced homology between the PRO537 amino acid sequence and the following Dayhoff sequences: A54523, CELF22H10_{—}2, FKH4_MOUSE, OTX1 HUMAN, URB1 USTMA, KNOB_PLAFN, A32895_{—}1, AF036332_{—}1, HRG_HUMAN and HRP3_PLAFS.

### Example 16

### Isolation of cDNA Clones Encoding Human PRO718 Polypeptides [UNQ386]

A cDNA sequence isolated in the amylase screen described in Example 2 (human fetal lung library) above is herein designated DNA43512. The DNA43512 sequence was then compared to a variety of expressed sequence tag (EST) databases which included public EST databases (e.g., GenBank) and a proprietary EST DNA database (LIFESEQ®, Incyte Pharmaceuticals, Palo Alto, Calif.) to identify existing homologies. The homology search was performed using the computer program BLAST or BLAST2 (Altshul et al., Methods in Enzymology 266:460-480 (1996)). Those comparisons resulting in a BLAST score of 70 (or in some cases 90) or greater that did not encode known proteins were clustered and assembled into consensus DNA sequences with the program “phrap” (Phil Green, University of Washington, Seattle, Wash.). The consensus sequence obtained therefrom is herein designated DNA45625. Proprietary Genentech EST sequences were employed in the assembly.

Based on the DNA45625 sequence, oligonucleotide probes were generated and used to screen a human fetal lung library (LIB25) prepared as described in paragraph 1 of Example 2 above. The cloning vector was pRK5B (pRK5B is a precursor of pRK5D that does not contain the SfiI site; see, Holmes et al., Science, 253:1278-1280 (1991)), and the cDNA size cut was less than 2800 bp.

PCR primers (forward and reverse) were synthesized:

| | |
|---|---|
| forward PCR primer | |
| 5′-GGGTGGATGGTACTGCTGCATCC-3′ | (SEQ ID NO: 192) |
| reverse PCR primer | |
| 5′-TGTTGTGCTGTGGGAAATCAGATGTG-3′ | (SEQ ID NO: 193) |

Additionally, a synthetic oligonucleotide hybridization probe was constructed from the DNA45625 sequence which had the following nucleotide sequence:

hybridization probe
(SEQ ID NO: 194)
5′-GTGTCTGGAGGCTGTGGCCGTTTTGTTTTCTTGGGCTAAAATCG
GG-3′

In order to screen several libraries for a source of a full-length clone, DNA from the libraries was screened by PCR amplification with the PCR primer pair identified above. A positive library was then used to isolate clones encoding the PRO718 gene using the probe oligonucleotide and one of the PCR primers.

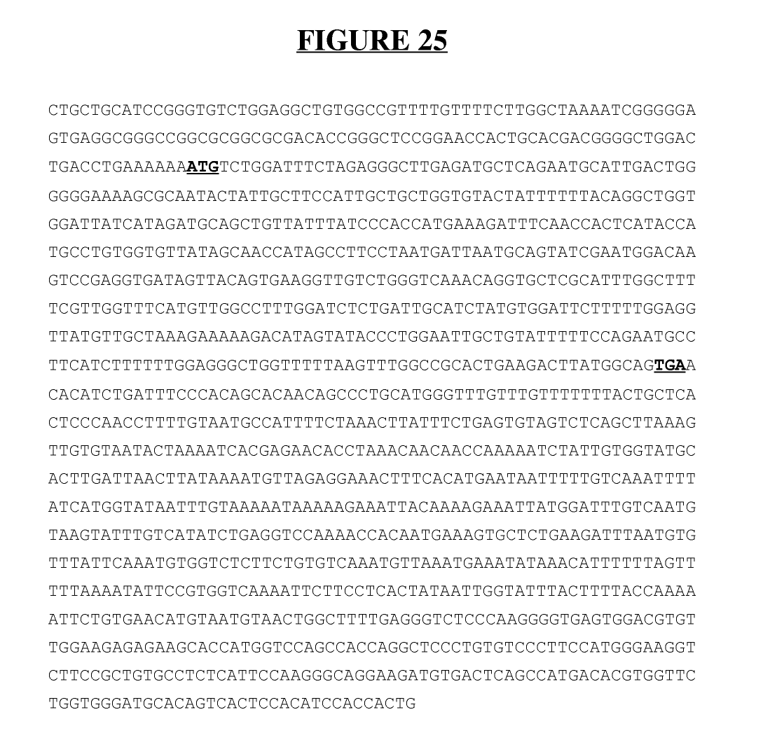

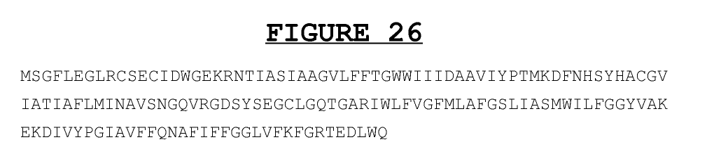

A full length clone was identified that contained a single open reading frame with an apparent translational initiation site at nucleotide positions 36-38 and ending at the stop codon found at nucleotide positions 607-609 (FIG. 25; SEQ ID NO:25). The predicted polypeptide precursor is 157 amino acids long, has a calculated molecular weight of approximately 17,400 daltons and an estimated pI of approximately 5.78. Analysis of the full-length PRO718 sequence shown in FIG. 26 (SEQ ID NO:26) evidences the presence of the following: a type II transmembrane domain from about amino acid 21 to about amino acid 40, and other transmembrane domains at about amino acid 58 to about amino acid 78, about amino acid 95 to about amino acid 114, and about amino acid 127 to about amino acid 147; a cell attachment sequence from about amino acid 79 to about amino acid 81; and a potential N-glycosylation site from about amino acid 53 to about amino acid 56. Clone DNA49647-1398 has been deposited with ATCC on Jun. 2, 1998 and is assigned ATCC deposit no. 209919.

Analysis of the amino acid sequence of the full-length PRO718 polypeptide suggests that it possesses no significant sequence similarity to any known protein. However, an analysis of the Dayhoff database (version 35.45 SwissProt 35) evidenced some degree of homology between the PRO718 amino acid sequence and the following Dayhoff sequences: AF045606_{—}1, AF039906_{—}1, SPBC8D2_{—}2, S63441, F64728, COX1_TRYBB, F64375, E64173, RPYGJT_{—}3, MTCY261_{—}23.

### Example 17

### Isolation of cDNA Clones Encoding Human PRO773 Polypeptides [UNQ411]

The extracellular domain (ECD) sequences (including the secretion signal sequence, if any) from about 950 known secreted proteins from the Swiss-Prot public database were used to search EST databases. The EST databases included (1) a proprietary EST database (LIFESEQ®, Incyte Pharmaceuticals, Palo Alto, Calif.) and (2) a proprietary EST database from Genentech. The search was performed using the computer program BLAST or BLAST2 [Altschul et al., Methods in Enzymology, 266:460-480 (1996)] as a comparison of the ECD protein sequences to a 6 frame translation of the EST sequences. Those comparisons resulting in a BLAST score of 70 (or in some cases, 90) or greater that did not encode known proteins were clustered and assembled into consensus DNA sequences with the program “phrap” (Phil Green, University of Washington, Seattle, Wash.).

A consensus DNA sequence was assembled relative to other EST sequences using phrap as described above. This consensus sequence is herein designated DNA40751. In some cases, the consensus sequence derives from an intermediate consensus DNA sequence which was extended using repeated cycles of BLAST and phrap to extend that intermediate consensus sequence as far as possible using the sources of EST sequences discussed above.

Based on the DNA40751 consensus sequence oligonucleotides were synthesized: 1) to identify by PCR a cDNA library that contained the sequence of interest, and 2) for use as probes to isolate a clone of the full-length coding sequence for PRO773. Forward and reverse PCR primers generally range from 20 to 30 nucleotides and are often designed to give a PCR product of about 100-1000 by in length. The probe sequences are typically 40-55 by in length. In some cases, additional oligonucleotides are synthesized when the consensus sequence is greater than about 1-1.5 kbp. In order to screen several libraries for a full-length clone, DNA from the libraries was screened by PCR amplification, as per Ausubel et al., Current Protocols in Molecular Biology, supra, with the PCR primer pair. A positive library was then used to isolate clones encoding the gene of interest using the probe oligonucleotide and one of the primer pairs.

PCR primers (forward and reverse) were synthesized:

| | |
|---|---|
| forward PCR primer | |
| 5′-CCTGGGCTCTGGCTCTTCTTTCAG-3′ | (SEQ ID NO: 195) |
| reverse PCR primer | |
| 5′-CCACTCAGAGGCCTCAGCTTTTCC-3′ | (SEQ ID NO: 196) |

Additionally, a synthetic oligonucleotide hybridization probe was constructed from the consensus DNA40751 sequence which had the following nucleotide sequence

hybridization probe
(SEQ ID NO: 197)
5′-TTTCGGCCACCCAGGCACGGAAAGGCTTCTGGGACTACTTCAG
CC-3′

RNA for construction of the cDNA libraries was isolated from human fetal liver tissue. The cDNA libraries used to isolate the cDNA clones were constructed by standard methods using commercially available reagents such as those from Invitrogen, San Diego, Calif. The cDNA was primed with oligo dT containing a NotI site, linked with blunt to SalI hemikinased adaptors, cleaved with NotI, sized appropriately by gel electrophoresis, and cloned in a defined orientation into a suitable cloning vector (such as pRK5B or pRK5D; pRK5B is a precursor of pRK5D that does not contain the SfiI site; see, Holmes et al., Science, 253:1278-1280 (1991)) in the unique XhoI and NotI sites.

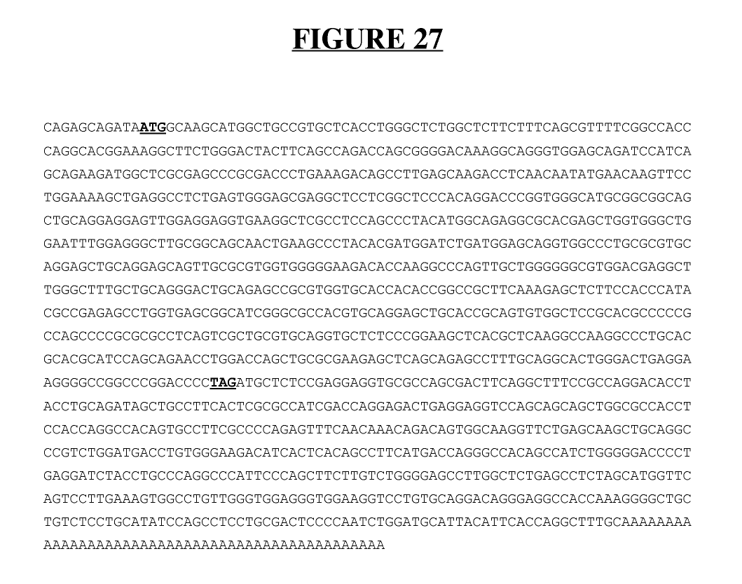

DNA sequencing of the clones isolated as described above gave the full-length DNA sequence for a full-length PRO773 polypeptide (designated herein as DNA48303-2829 [FIG. 27, SEQ ID NO: 27]) and the derived protein sequence for that PRO773 polypeptide.

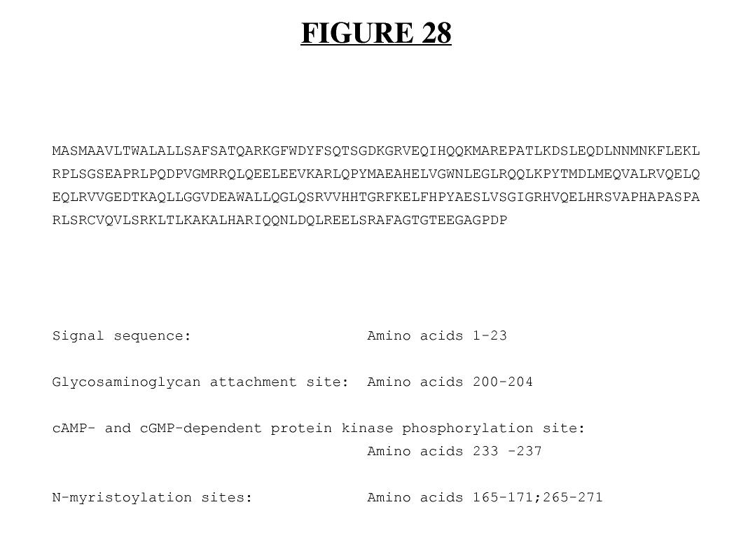

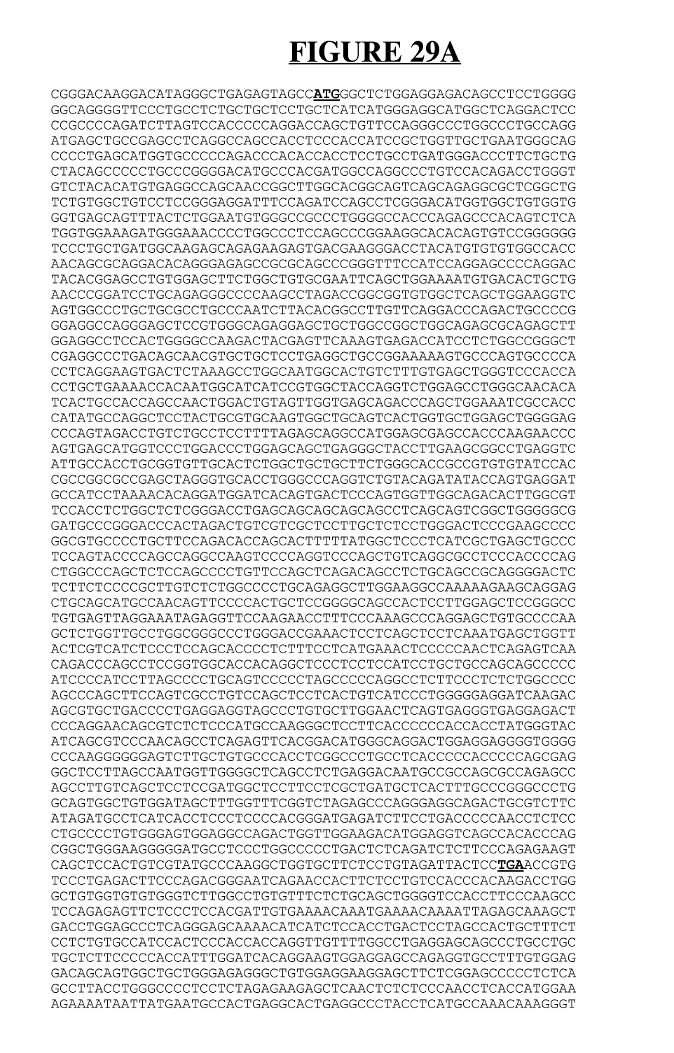

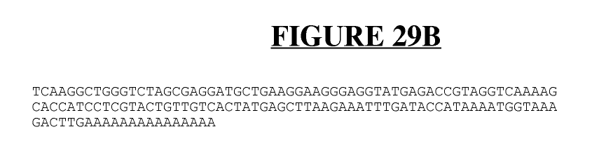

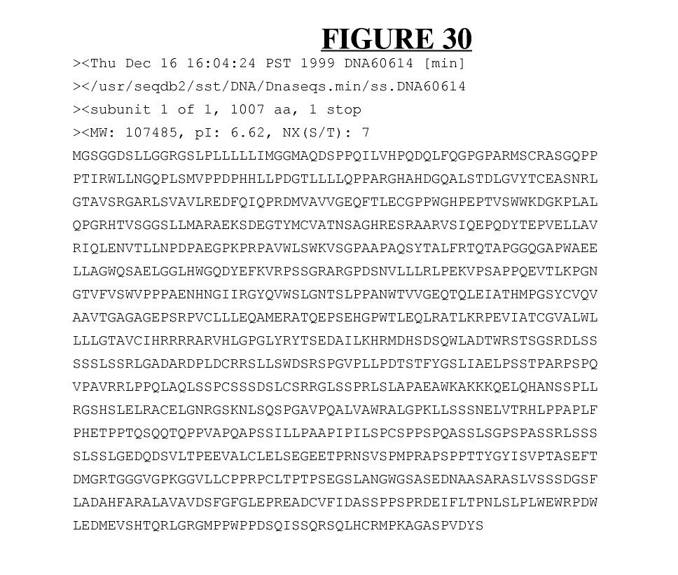

The full length clone identified above contained a single open reading frame with an apparent translational initiation site at nucleotide positions 12-14 and a stop signal at nucleotide positions 834-836 (FIG. 27, SEQ ID NO:27). The predicted polypeptide precursor is 274 amino acids long, has a calculated molecular weight of approximately 30754 daltons and an estimated pI of approximately 7.77. Analysis of the full-length PRO773 sequence shown in FIG. 28 (SEQ ID NO:28) evidences the presence of a variety of important polypeptide domains as shown in FIG. 28, wherein the locations given for those important polypeptide domains are approximate as described above. Chromosome mapping evidences that the PRO773-encoding nucleic acid maps to chromosome 11q23 in humans. Clone DNA48303-2829 has been deposited with ATCC on Feb. 8, 2000 and is assigned ATCC deposit no. PTA-1342.

An analysis of the Dayhoff database (version 35.45 SwissProt 35), using the ALIGN-2 sequence alignment analysis of the full-length sequence shown in FIG. 28 (SEQ ID NO:28), evidenced sequence identity between the PRO773 amino acid sequence and the following Dayhoff sequences: APA4_PIG, APA4_MACFA, APA4_HUMAN, APA4_PAPAN, P_R45244, P_R39501, P_R39499, APA4_RAT, APA4_MOUSE and P_R34032.

### Example 18

### Isolation of cDNA Clones Encoding Human PRO871 Polypeptides [UNQ438]

A consensus sequence was obtained relative to a variety of EST sequences as described in Example 1 above, wherein the consensus sequence obtained is herein designated DNA40324. Based on the DNA40324 consensus sequence, oligonucleotides were synthesized: 1) to identify by PCR a cDNA library that contained the sequence of interest, and 2) for use as probes to isolate a clone of the full-length coding sequence for PRO871.

PCR primers (forward and reverse) were synthesized:

| | |
|---|---|
| forward PCR primer 1 | |
| 5′-TGCGGAGATCCTACTGGCACAGGG-3′ | (SEQ ID NO: 198) |
| forward PCR primer 2 | |
| 5′-CGAGTTAGTCAGAGCATG-3′ | (SEQ ID NO: 199) |
| forward PCR primer 3 | |
| 5′-CAGATGGTGCTGTTGCCG-3′ | (SEQ ID NO: 200) |
| reverse PCR primer 1 | |
| 5′-CAACTGGAACAGGAACTGAGATGTGGATC-3′ | (SEQ ID NO: 201) |
| reverse PCR primer 2 | |
| 5′-CTGGTTCAGCAGTGCAAGGGTCTG-3′ | (SEQ ID NO: 202) |
| reverse PCR primer 3 | |
| 5′-CCTCTCCGATTAAAACGC-3′ | (SEQ ID NO: 203) |

Additionally, a synthetic oligonucleotide hybridization probe was constructed from the consensus DNA40324 sequence which had the following nucleotide sequence

hybridization probe
(SEQ ID NO: 204)
5′-GAGAGGACTGGTTGCCATGGCAAATGCTGGTTCTCATGATAATGG-3′

In order to screen several libraries for a source of a full-length clone, DNA from the libraries was screened by PCR amplification with one of the PCR primer pairs identified above. A positive library was then used to isolate clones encoding the PRO871 gene using the probe oligonucleotide and one of the PCR primers. RNA for construction of the cDNA libraries was isolated from human fetal kidney tissue (LIB227).

DNA sequencing of the clones isolated as described above gave the full-length DNA sequence for PRO871 [herein designated as UNQ438 (DNA50919-1361)] (SEQ ID NO:31) and the derived protein sequence for PRO871.

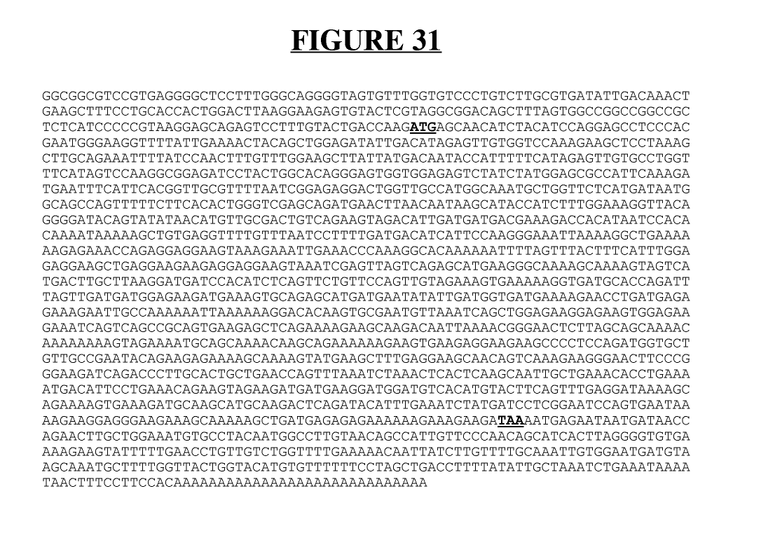

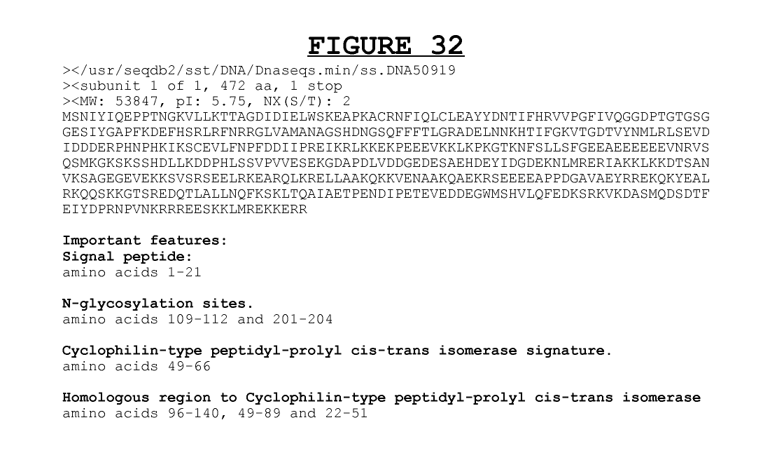

The entire nucleotide sequence of UNQ438 (DNA50919-1361) is shown in FIG. 31 (SEQ ID NO:31). Clone UNQ438 (DNA50919-1361) contains a single open reading frame with an apparent translational initiation site at nucleotide positions 191-193 and ending at the stop codon at nucleotide positions 1607-1609 (FIG. 31). The predicted polypeptide precursor is 472 amino acids long (FIG. 32; SEQ ID NO:32). The full-length PRO871 protein shown in FIG. 32 has an estimated molecular weight of about 53,847 daltons and a pI of about 5.75. Analysis of the full-length PRO871 sequence shown in FIG. 32 (SEQ ID NO:32) evidences the presence of the following: a signal peptide from about amino acid 1 to about amino acid 21, potential N-glycosylation sites from about amino acid 109 to about amino acid 112 and from about amino acid 201 to about amino acid 204, a cyclophilin-type peptidy-prolyl cis-trans isomerase signature sequence from about amino acid 49 to about amino acid 66 and regions that are homologous to cyclophilin-type peptidy-prolyl cis-trans isomerases from about amino acid 96 to about amino acid 140, from about amino acid 49 to about amino acid 89 and from about amino acid 22 to about amino acid 51. Clone UNQ438 (DNA50919-1361) has been deposited with ATCC on May 6, 1998 and is assigned ATCC deposit no. 209848.

Analysis of the amino acid sequence of the full-length PRO871 polypeptide suggests that it possesses significant sequence similarity to the cyclophilin family of proteins, thereby indicating that PRO871 may be a novel cyclophilin protein family member. More specifically, an analysis of the Dayhoff database (version 35.45 SwissProt 35) evidenced significant homology between the PRO871 amino acid sequence and the following Dayhoff sequences, SPBC16H5_{—}5, S64705, YAL5_SCHPO, CYP4_CAEEL, CELC34D4_{—}7, CYPA_CAEEL, HUMORF006_{—1}, CYPI_MYCTU, AF043642_{—}1 and HSSRCYP_{—}1.

### Example 19

### Isolation of cDNA Clones Encoding Human PRO872 Polypeptides [UNQ439]

Use of the signal sequence algorithm described in Example 3 above allowed identification of a single Incyte EST sequence designated herein as clu120709.init. The clu120709.init sequence was then compared a proprietary EST DNA database (LIFESEQ®, Incyte Pharmaceuticals, Palo Alto, Calif.) to identify existing homologies. The homology search was performed using the computer program BLAST or BLAST2 (Altshul et al., Methods in Enzymology 266:460-480 (1996)). Those comparisons resulting in a BLAST score of 70 (or in some cases 90) or greater that did not encode known proteins were clustered and assembled into a consensus DNA sequence with the program “phrap” (Phil Green, University of Washington, Seattle, Wash.). The consensus sequence obtained therefrom is herein designated DNA48254.

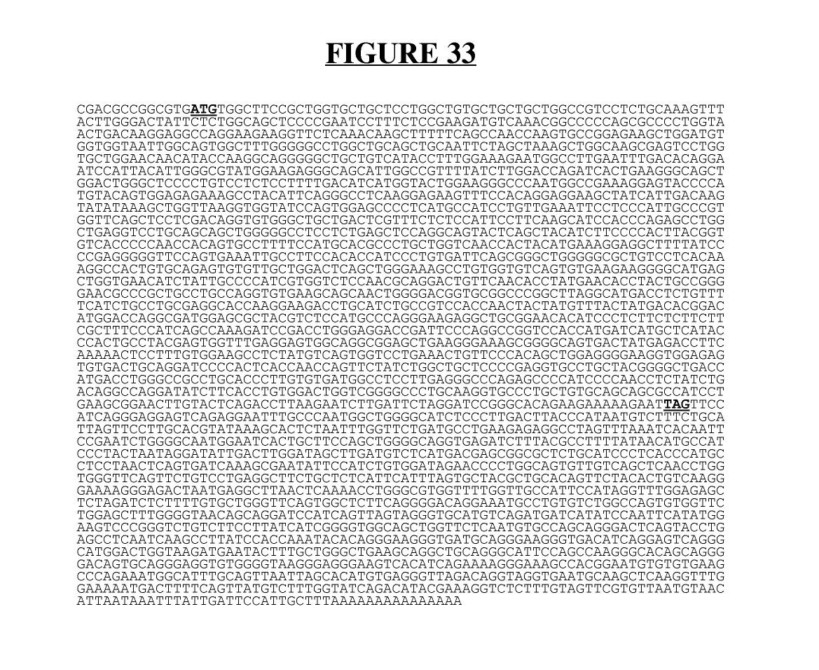

In light of an observed sequence homology between the DNA48254 consensus sequence and an EST sequence encompassed within the Incyte EST clone no. 3438068, the Incyte EST clone 3438068 was purchased and the cDNA insert was obtained and sequenced. It was found that this insert encoded a full-length protein. The sequence of this cDNA insert is shown in FIG. 33 and is the full-length DNA sequence for PRO872. Clone DNA49819-1439 was deposited with the ATCC on Jun. 2, 1998, and is assigned ATCC deposit no. 209931.

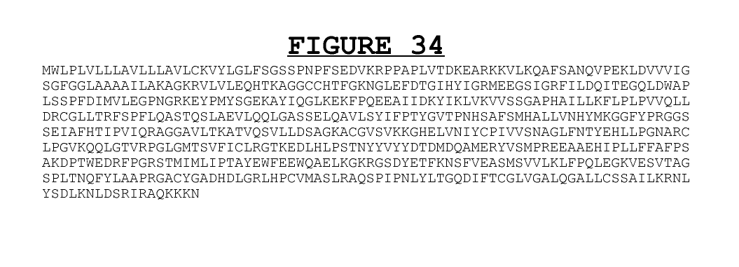

The entire nucleotide sequence of DNA49819-1439 is shown in FIG. 33 (SEQ ID NO:33). Clone DNA49819-1439 contains a single open reading frame with an apparent translational initiation site at nucleotide positions 14-16 and ending at the stop codon at nucleotide positions 1844-1846 (FIG. 33). The predicted polypeptide precursor is 610 amino acids long (FIG. 34; SEQ ID NO:34). The full-length PRO872 protein shown in FIG. 34 has an estimated molecular weight of about 66,820 daltons and a pI of about 8.65. Analysis of the full-length PRO872 sequence shown in FIG. 34 (SEQ ID NO:34) evidences the presence of the following features: a signal peptide at amino acid 1 to about 18, putative transmembrane domains at about amino acids 70-87, 200-222 and 568-588; sequence identity with bacterial-type phytoene dehydrogenase protein at about amino acids 71-105; sequence identity with a regulator of chromosome condensation (RCC1) signature 2 at about amino acids 201-211; leucine zipper patterns at about amino acids 214-235, 221-242, 228-249 and 364-385; a potential N-glycosylation site at about amino acids 271-274; and a glycosaminoglycan attachment site at about amino acids 75-78. Analysis of the amino acid sequence of the full-length PRO872 polypeptide using the Dayhoff database (version 35.45 SwissProt 35) evidenced homology between the PRO872 amino acid sequence and the following Dayhoff sequences: PRCRTI_{—}1, S75951, S74689, CELF37C4_{—}3, CRTI_RHOCA, S76617, YNI2_METTL, MTV014_{—}14, AOFB_HUMAN, and MMU70429_{—}1.

### Example 20

### Isolation of cDNA Clones Encoding Human PRO813 Polypeptides [UNQ465]

Use of the signal sequence algorithm described in Example 3 above allowed identification of a single Incyte EST cluster sequence (Incyte EST cluster sequence no. 45501. The Incyte EST cluster sequence no. 45501 sequence was then compared to a variety of expressed sequence tag (EST) databases which included public EST databases (e.g., GenBank) and a proprietary EST DNA database (LIFESEQ®, Incyte Pharmaceuticals, Palo Alto, Calif.) to identify existing homologies. The homology search was performed using the computer program BLAST or BLAST2 (Altshul et al., Methods in Enzymology 266:460-480 (1996)). Those comparisons resulting in a BLAST score of 70 (or in some cases 90) or greater that did not encode known proteins were clustered and assembled into a consensus DNA sequence with the program “phrap” (Phil Green, University of Washington, Seattle, Wash.). The consensus sequence obtained therefrom is herein designated DNA56400.

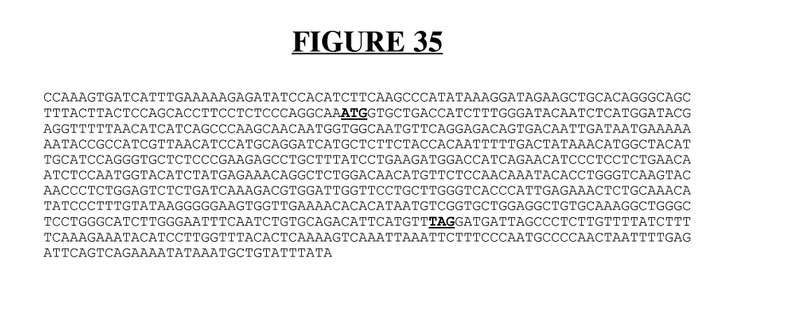

In light of an observed sequence homology between the DNA56400 consensus sequence and an EST sequence encompassed within the Merck EST clone no. T90592, the Merck EST clone T90592 was purchased and the cDNA insert was obtained and sequenced. It was found that this insert encoded a full-length protein. The sequence of this cDNA insert is shown in FIG. 35 and is herein designated DNA57834-1339.

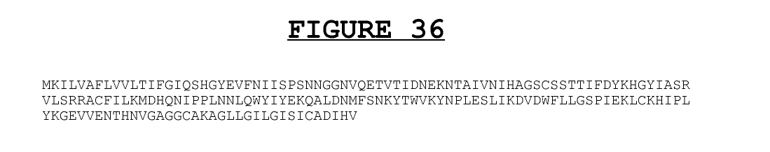

The full length clone shown in FIG. 35 contained a single open reading frame with an apparent translational initiation site at nucleotide positions 109-111 and ending at the stop codon found at nucleotide positions 637-639 (FIG. 35; SEQ ID NO:35). The predicted polypeptide precursor is 176 amino acids long, has a calculated molecular weight of approximately 19,616 daltons and an estimated pI of approximately 7.11. Analysis of the full-length PRO813 sequence shown in FIG. 36 (SEQ ID NO:36) evidences the presence of the following: a signal peptide from about amino acid 1 to about amino acid 26 and potential N-myristoylation sites from about amino acid 48 to about amino acid 53, from about amino acid 153 to about amino acid 158, from about amino acid 156 to about amino acid 161 and from about amino acid 167 to about amino acid 172. Clone DNA57834-1339 has been deposited with the ATCC on Jun. 9, 1998 and is assigned ATCC deposit no. 209954.

Analysis of the amino acid sequence of the full-length PRO813 polypeptide suggests that it possesses sequence similarity to the pulmonary surfactant-associated protein C. More specifically, an analysis of the Dayhoff database (version 35.45 SwissProt 35) evidenced some degree of homology between the PRO813 amino acid sequence and the following Dayhoff sequences, PSPC_MUSV₁, P_P92071, G02964, P_R65489, P_P82977, P_R84555, S55542, MUSIGHAJ_{—}1 and PH1158.

### Example 21

### Isolation of cDNA Clones Encoding Human PRO828 Polypeptides [UNQ469]

A consensus DNA sequence was identified using the method described in Example 1 above. This consensus sequence is herein designated DNA35717. Based on the DNA35717 consensus sequence, oligonucleotides were synthesized: 1) to identify by PCR a cDNA library that contained the sequence of interest, and 2) for use as probes to isolate a clone of the full-length coding sequence for PRO828.

PCR primers (forward and reverse) were synthesized:

| | |
|---|---|
| forward PCR primer | |
| 5′-GCAGGACTTCTACGACTTCAAGGC-3′; | (SEQ ID NO: 205) |
| and | |
| reverse PCR primer | |
| 5′-AGTCTGGGCCAGGTACTTGAAGGC-3′. | (SEQ ID NO: 206) |

Additionally, a synthetic oligonucleotide hybridization probe was constructed from the consensus DNA35717 sequence which had the following nucleotide sequence:

hybridization probe
(SEQ ID NO: 207)
5′-CAACATCCGGGGCAAACTGGTGTCGCTGGAGAAGTACCGCGGATC
GGTGT-3′

In order to screen several libraries for a source of a full-length clone, DNA from the libraries was screened by PCR amplification with the PCR primer pair identified above. A positive library was then used to isolate clones encoding the PRO828 gene using the probe oligonucleotide and one of the PCR primers. RNA for construction of the cDNA libraries was isolated from human fetal lung tissue (LIB25).

DNA sequencing of the clones isolated as described above gave the full-length DNA sequence for PRO828 [herein designated as DNA57037-1444] (SEQ ID NO:37) and the derived protein sequence for PRO828.

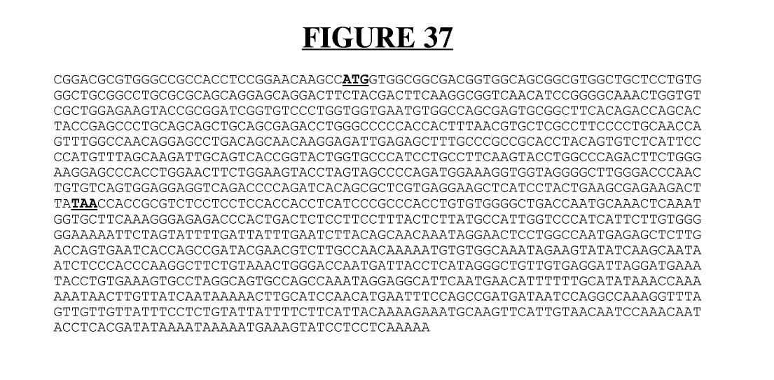

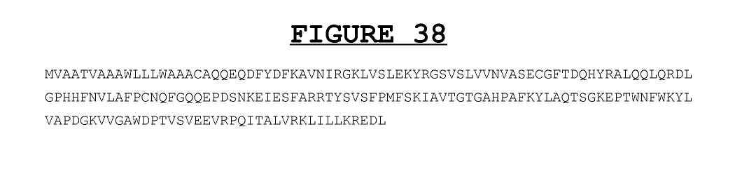

The entire nucleotide sequence of DNA57037-1444 is shown in FIG. 37 (SEQ ID NO:37). Clone DNA57037-1444 contains a single open reading frame with an apparent translational initiation site at nucleotide positions 34-36 and ending at the stop codon at nucleotide positions 595-597 (FIG. 37). The predicted polypeptide precursor is 187 amino acids long (FIG. 38; SEQ ID NO:38). The full-length PRO828 protein shown in FIG. 38 has an estimated molecular weight of about 20,996 daltons and a pI of about 8.62. Analysis of the full-length PRO828 sequence shown in FIG. 38 (SEQ ID NO:38) evidences the presence of the following: a signal peptide at about amino acids 1-21; sequences identity to glutathione peroxidases signature 2 at about amino acids 82-89; sequence identity to glutathione peroxidases selenocysteine proteins at about amino acids 35-60, 63-100, 107-134, and 138-159. Clone DNA57037-1444 has been deposited with ATCC on May 27, 1998, and is assigned ATCC deposit no. 209903.

Analysis of the amino acid sequence of the full-length PRO828 polypeptide suggests that it possesses significant sequence similarity to glutathione peroxidases, thereby indicating that PRO828 may be a novel peroxidase enzyme. More specifically, an analysis of the Dayhoff database (version 35.45 SwissProt 35) evidenced sequence identity between the PRO828 amino acid sequence and the following Dayhoff sequences: AF053311_{—}1, CELT09A12_{—}2, AC004151_{—}3, BTUE ECOLI, CER05H10_{—}3, P_P80918, PWU88907_{—}1, and P_W22308.

### Example 22

### Isolation of cDNA Clones Encoding Human PRO1100 Polypeptides 11 NO₅₄₆₁

Use of the signal sequence algorithm described in Example 3 above allowed identification of an EST cluster sequence from the Incyte database. This EST cluster sequence was then compared to a variety of expressed sequence tag (EST) databases which included public EST databases (e.g., GenBank) and a proprietary EST DNA database (Lifeseq®, Incyte Pharmaceuticals, Palo Alto, Calif.) to identify existing homologies. The homology search was performed using the computer program BLAST or BLAST2 (Altshul et al., Methods in Enzymology 266:460-480 (1996)). Those comparisons resulting in a BLAST score of 70 (or in some cases 90) or greater that did not encode known proteins were clustered and assembled into a consensus DNA sequence with the program “phrap” (Phil Green, University of Washington, Seattle, Wash.).

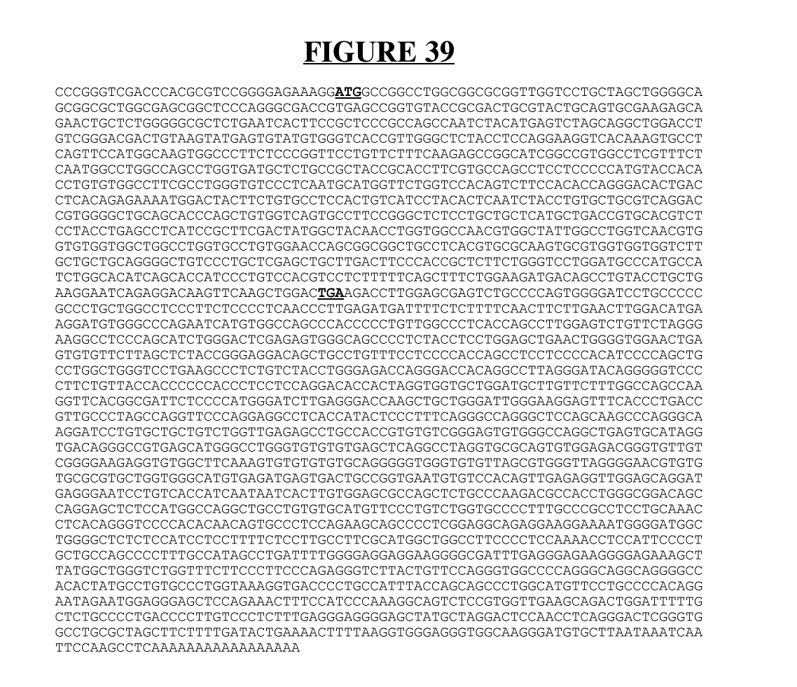

In light of an observed sequence homology between the obtained consensus sequence and an EST sequence encompassed within the Incyte EST clone no. 2305379, the Incyte EST clone 2305379 was purchased and the cDNA insert was obtained and sequenced. It was found that this insert encoded a full-length protein. The sequence of this cDNA insert is shown in FIG. 39 and is herein designated as DNA59619-1464.

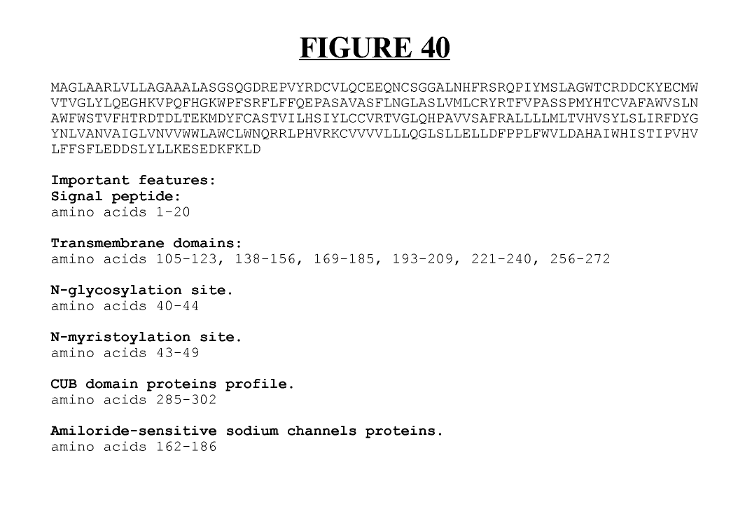

The entire nucleotide sequence of DNA59619-1464 is shown in FIG. 39 (SEQ ID NO:39). Clone DNA59619-1464 contains a single open reading frame with an apparent translational initiation site at nucleotide positions 33-35 and ending at the stop codon at nucleotide positions 993-995 of SEQ ID NO:39 (FIG. 39). The predicted polypeptide precursor is 320 amino acids long (FIG. 40; SEQ ID NO:40). The full-length PRO1100 protein shown in FIG. 40 has an estimated molecular weight of about 36,475 daltons and a pI of about 7.29. Clone DNA59619-1464 has been deposited with ATCC on Jul. 1, 1998 (ATCC deposit no. 203041). It is understood that the deposited clone has the actual nucleic acid sequence and that the sequences provided herein are based on known sequencing techniques.

Upon analyzing SEQ ID NO:40, the approximate locations of the signal peptide, the transmembrane domains, an N-glycosylation site, an N-myristoylation site, a CUB domain and an amiloride-sensitive sodium channel domain are present. It is believed that PRO1100 may function as a channel. The corresponding nucleic acids for these amino acids and others can be routinely determined given SEQ ID NO:40.

### Example 23

### Isolation of cDNA Clones Encoding Human PRO1114 Polypeptides [UN0557]

A cDNA sequence isolated in the amylase screen described in Example 2 was found, by the WU-BLAST2 sequence alignment computer program, to have certain sequence identity to other known interferon receptors. This cDNA sequence is herein designated DNA48466. Based on the sequence identity, probes were generated from the sequence of the DNA48466 molecule and used to screen a human breast carconoma library (LIB135) prepared as described in paragraph 1 of Example 2 above. The cloning vector was pRK5B (pRK5B is a precursor of pRK5D that does not contain the SfiI site; see, Holmes et al., Science, 253:1278-1280 (1991)), and the cDNA size cut was less than 2800 bp.

The oligonucleotide probes employed were as follows:

forward PCR primer
(SEQ ID NO: 208)
5′-AGGCTTCGCTGCGACTAGACCTC-3′
reverse PCR primer
(SEQ ID NO: 209)
5′-CCAGGTCGGGTAAGGATGGTTGAG-3′
hybridization probe
(SEQ ID NO: 210)
5′-TTTCTACGCATTGATTCCATGTTTGCTCACAGATGAAGTGGCCA
TTCTGC-3′

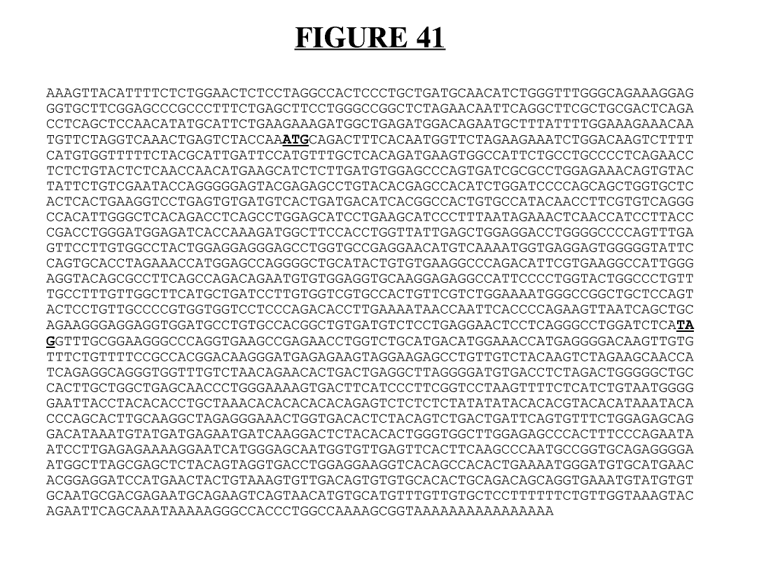

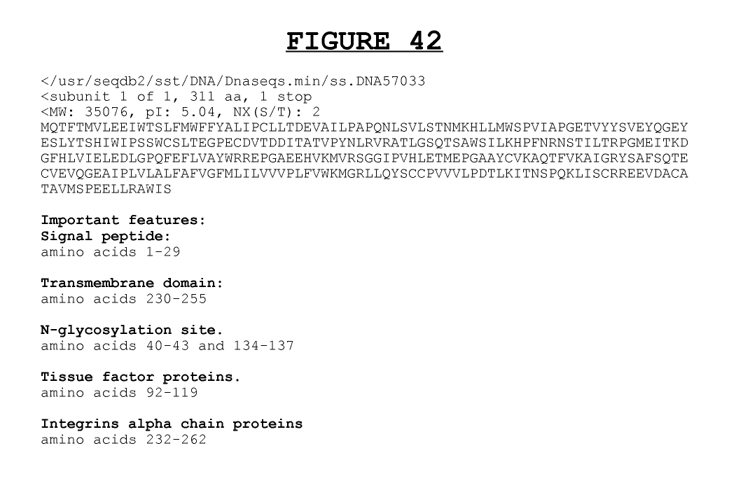

A full length clone was identified that contained a single open reading frame with an apparent translational initiation site at nucleotide positions 250-252, and a stop signal at nucleotide positions 1183-1185 (FIG. 41, SEQ ID NO:41). The predicted polypeptide precursor is 311 amino acids long, has a calculated molecular weight of approximately 35,076 daltons and an estimated pI of approximately 5.04. Analysis of the full-length PRO1114 interferon receptor sequence shown in FIG. 42 (SEQ ID NO:42) evidences the presence of the following: a signal peptide from about amino acid 1 to about amino acid 29, a transmembrane domain from about amino acid 230 to about amino acid 255, potential N-glycosylation sites from about amino acid 40 to about amino acid 43 and from about amino acid 134 to about amino acid 137, an amino acid sequence block having homology to tissue factor proteins from about amino acid 92 to about amino acid 119 and an amino acid sequence block having homology to integrin alpha chain proteins from about amino acid 232 to about amino acid 262. Clone UNQ557 (DNA57033-1403) has been deposited with ATCC on May 27, 1998 and is assigned ATCC deposit no. 209905.

An analysis of the Dayhoff database (version 35.45 SwissProt 35), using a WU-BLAST2 sequence alignment analysis of the full-length sequence shown in FIG. 42 (SEQ ID NO:42), evidenced significant homology between the PRO1114 interferon receptor amino acid sequence and the following Dayhoff sequences: G01418, INR1_MOUSE, P_R71035, INGS_HUMAN, A26595_{—}1, A26593_{—}1, I56215 and TF_HUMAN.

### Example 24

### Isolation of cDNA Clones Encoding Human PROMS Polypeptides [UN0558]

Use of the signal sequence algorithm described in Example 3 above allowed identification of an EST cluster sequence from the LIFESEQ® database, designated Incyte EST cluster sequence no. 165008. This EST cluster sequence was then compared to a variety of expressed sequence tag (EST) databases which included public EST databases (e.g., GenBank) and a proprietary EST DNA database (LIFESEQ®, Incyte Pharmaceuticals, Palo Alto, Calif.) to identify existing homologies. The homology search was performed using the computer program BLAST or BLAST2 (Altshul et al., Methods in Enzymology 266:460-480 (1996)). Those comparisons resulting in a BLAST score of 70 (or in some cases 90) or greater that did not encode known proteins were clustered and assembled into a consensus DNA sequence with the program “phrap” (Phil Green, University of Washington, Seattle, Wash.). The consensus sequence obtained therefrom is herein designated DNA55726.

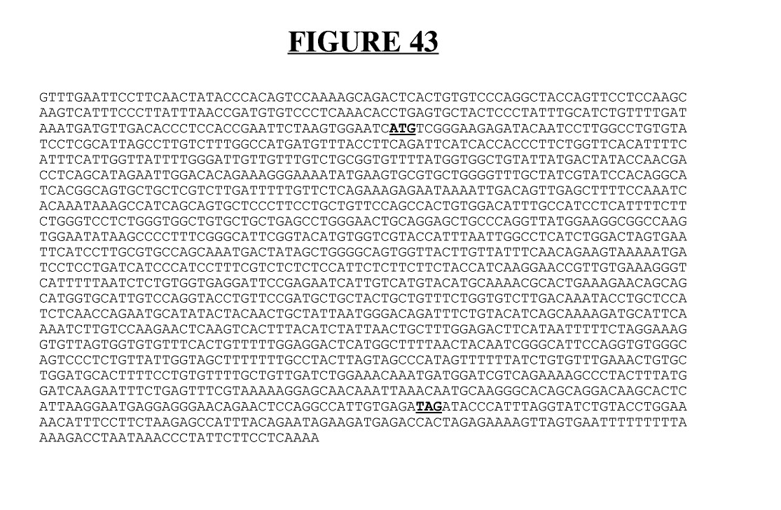

In light of an observed sequence homology between the DNA55726 consensus sequence and an EST sequence encompassed within the Merck EST clone no. R75784, the Merck EST clone R75784 was purchased and the cDNA insert was obtained and sequenced. It was found that this insert encoded a full-length protein. The sequence of this cDNA insert is shown in FIG. 43 and is herein designated as DNA56868-1478.

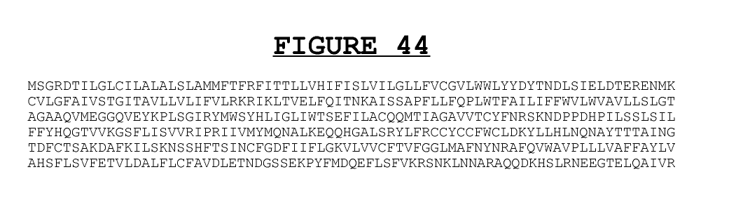

The full length clone shown in FIG. 43 contained a single open reading frame with an apparent translational initiation site at nucleotide positions 189-191 and ending at the stop codon found at nucleotide positions 1524-1526 (FIG. 43; SEQ ID NO:43). The predicted polypeptide precursor (FIG. 44, SEQ ID NO:44) is 445 amino acids long. PROMS has a calculated molecular weight of approximately 50,533 Daltons and an estimated pI of approximately 8.26. Additional features include a signal peptide at about amino acids 1-20; potential N-glycosylation sites at about amino acids 204-207, 295-298, and 313-316; and putative transmembrane domains at about amino acids 35-54, 75-97, 126-146, 185-204, 333-350, and 353-371.

An analysis of the Dayhoff database (version 35.45 SwissProt 35), using a WU-BLAST-2 sequence alignment analysis of the full-length sequence shown in FIG. 44 (SEQ ID NO:44), evidenced some amino acid sequence identity between the PROMS amino acid sequence and the following Dayhoff sequences: AF053947_{—}79, S73698, CEC47A10_{—}4, CCOMTNDS5G_{—}1, HS4LMP2AC_{—}1, LMP2_EBV, PA24_MOUSE, HCU33331_{—}7, P-WO5508, and AF002273_{—}1.

Clone DNA56868-1478 was deposited with the ATCC on Jun. 23, 1998 and is assigned ATCC deposit no. 203024.

### Example 25

### Isolation of cDNA Clones Encoding Human PRO1126 Polypeptides [UNQ564]

Use of the signal sequence algorithm described in Example 3 above allowed identification of a single EST cluster sequence from the Incyte database. This EST cluster sequence was then compared to a variety of expressed sequence tag (EST) databases which included public EST databases (e.g., GenBank) and a proprietary EST DNA database (LIFESEQ®, Incyte Pharmaceuticals, Palo Alto, Calif.) to identify existing homologies. The homology search was performed using the computer program BLAST or BLAST2 (Altshul et al., Methods in Enzymology 266:460-480 (1996)). Those comparisons resulting in a BLAST score of 70 (or in some cases 90) or greater that did not encode known proteins were clustered and assembled into a consensus DNA sequence with the program “phrap” (Phil Green, University of Washington, Seattle, Wash.). The consensus sequence obtained therefrom is herein designated DNA56250.

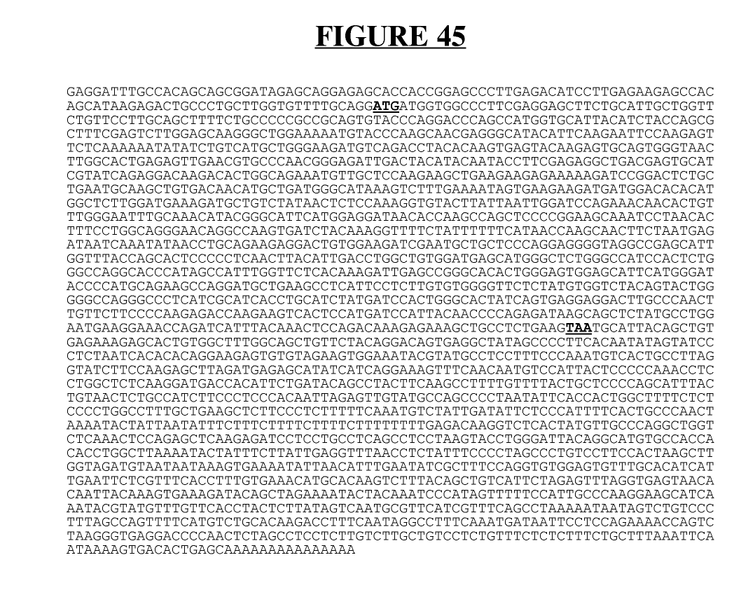

In light of an observed sequence homology between the DNA56250 consensus sequence and an EST sequence encompassed within the Incyte EST clone no. 1437250, the Incyte EST clone 1437250 was purchased and the cDNA insert was obtained and sequenced. It was found that this insert encoded a full-length protein. The sequence of this cDNA insert is shown in FIG. 45 and is herein designated as DNA60615-1483.

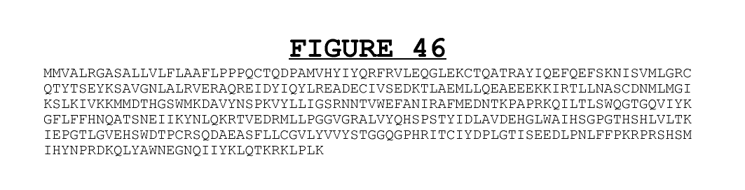

Clone DNA60615-1483 contains a single open reading frame with an apparent translational initiation site at nucleotide positions 110-112 and ending at the stop codon at nucleotide positions 1316-1318 (FIG. 45; SEQ ID NO:45). The predicted polypeptide precursor is 402 amino acids long (FIG. 46; SEQ ID NO:46). The full-length PRO1126 protein shown in FIG. 46 has an estimated molecular weight of about 45,921 daltons and a pI of about 8.60. Analysis of the full-length PRO1126 sequence shown in FIG. 46 (SEQ ID NO:46) evidences the presence of the following: a signal peptide from about amino acid 1 to about amino acid 25 and potential N-glycosylation sites from about amino acid 66 to about amino acid 69, from about amino acid 138 to about amino acid 141 and from about amino acid 183 to about amino acid 186. Clone DNA60615-1483 has been deposited with ATCC on Jun. 16, 1998 and is assigned ATCC deposit no. 209980.

An analysis of the Dayhoff database (version 35.45 SwissProt 35), using a WU-BLAST2 sequence alignment analysis of the full-length sequence shown in FIG. 46 (SEQ ID NO:46), evidenced significant homology between the PRO1126 amino acid sequence and the following Dayhoff sequences: I73636, NOMR_HUMAN, MMUSMYOC3_{—}1, HS454G6_{—}1, P_R98225, RNU78105_{—}1, RNU72487_{—}1, AF035301_{—}1, CEELC48E7_{—}4 and CEF11C3_{—}3.

### Example 26

### Isolation of cDNA Clones Encoding Human PRO1133 Polypeptides [UNQ571]

A consensus DNA sequence was assembled relative to other EST sequences using phrap as described in Example 1 above. This sequence was extended using repeated cycles of phrap. The extended consensus sequence is designated herein DNA38102. Based on the DNA38102 consensus sequence, oligonucleotides were synthesized: 1) to identify by PCR a cDNA library that contained the sequence of interest, and 2) for use as probes to isolate a clone of the full-length coding sequence for PRO1133.

PCR primers (two forward and one reverse) were synthesized:

| | |
|---|---|
| forward PCR primer 1 | |
| 5′-TCGATTATGGACGAACATGGCAGC-3′; | (SEQ ID NO: 211) |
| forward PCR primer 2 | |
| 5′-TTCTGAGATCCCTCATCCTC-3′; | (SEQ ID NO: 212) |
| and | |
| reverse primer | |
| 5′-AGGTTCAGGGACAGCAAGTTTGGG-3′. | (SEQ ID NO: 213) |

Additionally, a synthetic oligonucleotide hybridization probe was constructed from the consensus DNA38102 sequence which had the following nucleotide sequence:

hybridization probe
(SEQ ID NO: 214)
5′TTTGCTGGACCTCGGCTACGGAATTGGCTTCCCTCTACGGACAGCT
GGAT3′.

In order to screen several libraries for a source of a full-length clone, DNA from the libraries was screened by PCR amplification with a PCR primer pair identified above. A positive library was then used to isolate clones encoding the PRO1133 gene using the probe oligonucleotide and one of the PCR primers. RNA for construction of the cDNA libraries was isolated from human fetal kidney tissue.

DNA sequencing of the clones isolated as described above gave the full-length DNA sequence for PRO1133 and the derived protein sequence for PRO1133.

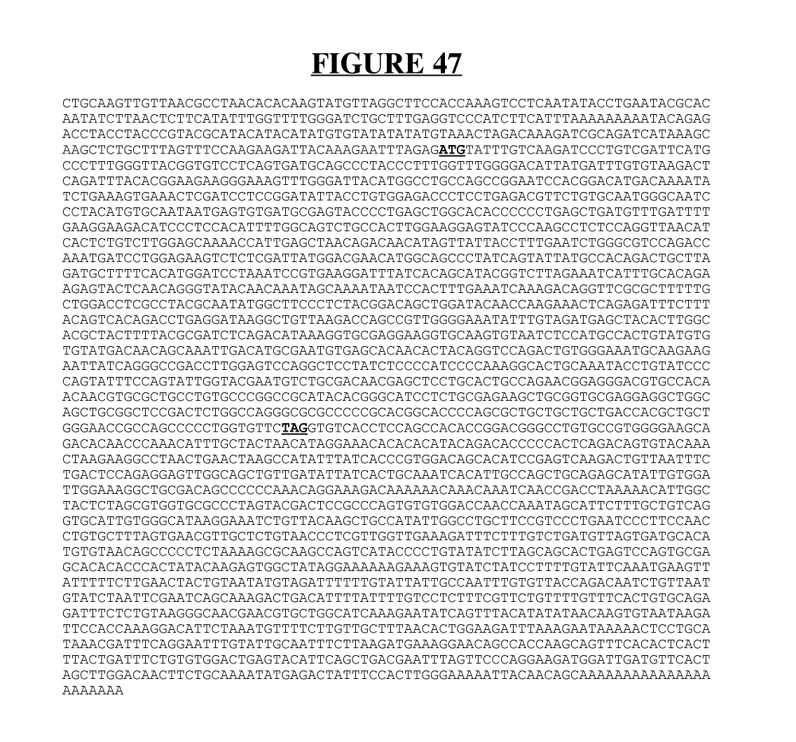

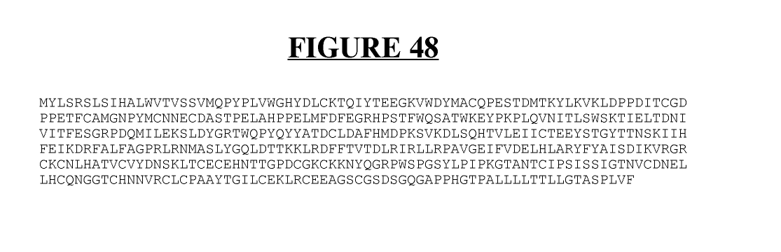

The entire coding sequence of PRO1133 is shown in FIG. 47 (SEQ ID NO:47). Clone DNA53913-1490 contains a single open reading frame with an apparent translational initiation site at nucleotide positions 266-268 and an apparent stop codon at nucleotide positions 1580-1582 of SEQ ID NO:47. The predicted polypeptide precursor is 438 amino acids long (FIG. 48; SEQ ID NO:48). The signal peptide is at amino acids 1-18 of SEQ ID NO:48. EGF-like domain cysteine pattern signatures start at 315 and 385 of SEQ ID NO:48 as shown in FIG. 48. Clone DNA53913-1490 has been deposited with ATCC (Aug. 25, 1998) and is assigned ATCC deposit no. 203162. The full-length PRO1133 protein shown in FIG. 48 has an estimated molecular weight of about 49,260 daltons and a pI of about 6.15.

An analysis of the Dayhoff database (version 35.45 SwissProt 35), using a WU-BLAST-2 sequence alignment analysis of the full-length sequence shown in FIG. 48 (SEQ ID NO:48), revealed some sequence identity between the PRO1133 amino acid sequence and the following Dayhoff sequences (data from the database incorporated herein): AF002717_{—}1, LMG1_HUMAN, B54665, UNC6_CAEEL, LML1_CAEEL, LMA5_MOUSE, MMU88353_{—}1, LMA1_HUMAN, HSLN2C64_{—}1 and AF005258_{—}1.

### Example 27

### Isolation of cDNA Clones Encoding Human PRO1154 Polypeptides [UNQ584]

Use of the signal sequence algorithm described in Example 3 above allowed identification of a single EST cluster sequence from the Incyte database. This EST cluster sequence was then compared to a variety of expressed sequence tag (EST) databases which included public EST databases (e.g., GenBank) and a proprietary EST DNA database (LIFESEQ®, Incyte Pharmaceuticals, Palo Alto, Calif.) to identify existing homologies. The homology search was performed using the computer program BLAST or BLAST2 (Altshul et al., Methods in Enzymology 266:460-480 (1996)). Those comparisons resulting in a BLAST score of 70 (or in some cases 90) or greater that did not encode known proteins were clustered and assembled into a consensus DNA sequence with the program “phrap” (Phil Green, University of Washington, Seattle, Wash.). The consensus sequence obtained therefrom is herein designated DNA56025.

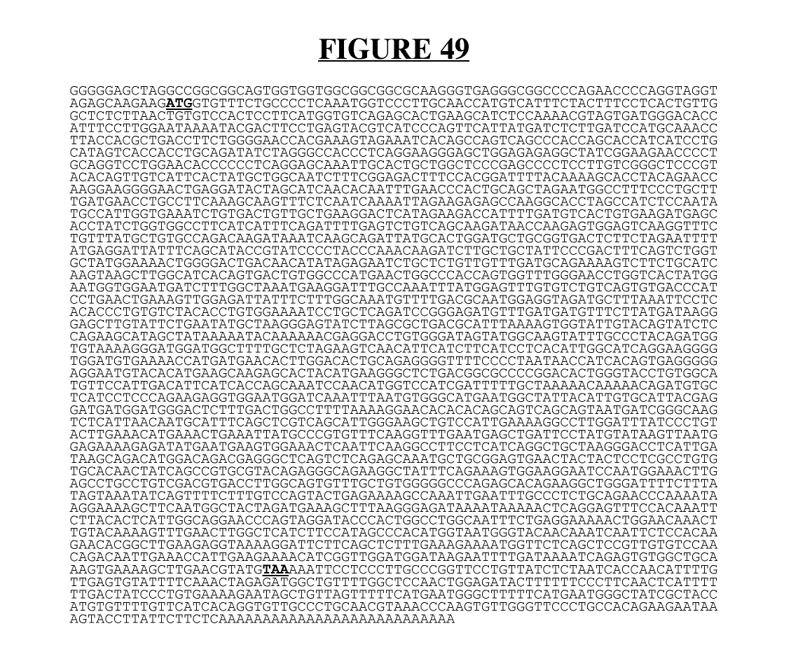

In light of an observed sequence homology between the DNA56025 consensus sequence and an EST sequence encompassed within the Incyte EST clone no. 2169375, the Incyte EST clone 2169375 was purchased and the cDNA insert was obtained and sequenced. It was found that this insert encoded a full-length protein. The sequence of this cDNA insert is shown in FIG. 49 and is herein designated as DNA59846-1503.

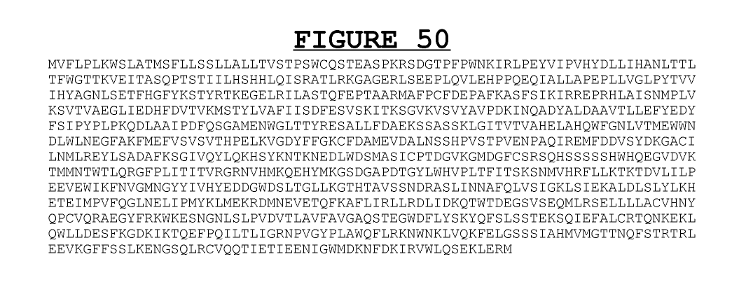

The full length clone shown in FIG. 49 contained a single open reading frame with an apparent translational initiation site at nucleotide positions 86-88 and ending at the stop codon found at nucleotide positions 2909-2911 (FIG. 49; SEQ ID NO:49). The predicted polypeptide precursor (FIG. 50, SEQ ID NO:50) is 941 amino acids long. PRO1154 has a calculated molecular weight of approximately 107,144 daltons and an estimated pI of approximately 6.26. Clone DNA59846-1503 has been deposited with ATCC (Jun. 16, 1998) and is assigned ATCC deposit no. 209978.

Based on a WU-BLAST2 sequence alignment analysis (using the ALIGN computer program) of the full-length sequence, PRO1154 shows sequence identity to at least the following Dayhoff designations: AB011097_{—}1, AMPN_HUMAN, RNU76997_{—}1, 159331, GEN14047, HSU62768_{—}1, P_R51281, CET07F10_{—}1, SSU66371_{—}1, and AMPRE_HUMAN.

### Example 28

### Isolation of cDNA Clones Encoding Human PRO1185 Polypeptides [UNQ599]

Use of the signal sequence algorithm described in Example 3 above allowed identification of a single EST cluster sequence from the Incyte database. This EST cluster sequence was then compared to a variety of expressed sequence tag (EST) databases which included public EST databases (e.g., GenBank) and a proprietary EST DNA database (LIFESEQ®, Incyte Pharmaceuticals, Palo Alto, Calif.) to identify existing homologies. The homology search was performed using the computer program BLAST or BLAST2 (Altshul et al., Methods in Enzymology 266:460-480 (1996)). Those comparisons resulting in a BLAST score of 70 (or in some cases 90) or greater that did not encode known proteins were clustered and assembled into a consensus DNA sequence with the program “phrap” (Phil Green, University of Washington, Seattle, Wash.). The consensus sequence obtained therefrom is herein designated DNA56426.

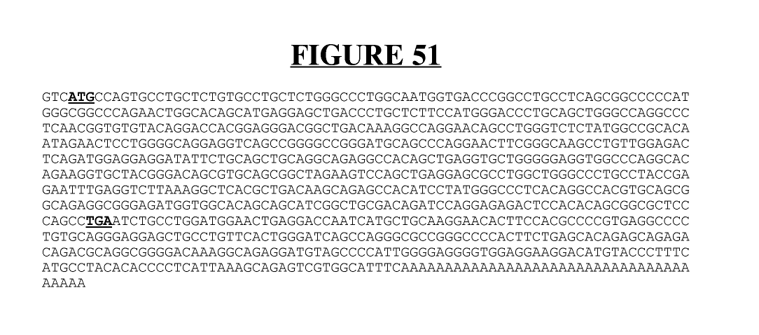

In light of an observed sequence homology between the DNA56426 consensus sequence and an EST sequence encompassed within the Incyte EST clone no. 3284411, the Incyte EST clone 3284411 was purchased and the cDNA insert was obtained and sequenced. It was found that this insert encoded a full-length protein. The sequence of this cDNA insert is shown in FIG. 51 and is herein designated as DNA62881-1515.

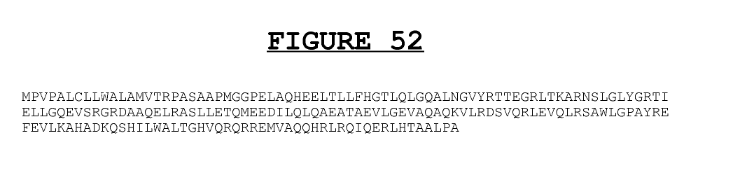

The full length DNA62881-1515 clone shown in FIG. 51 contained a single open reading frame with an apparent translational initiation site at nucleotide positions 4-6 and ending at the stop codon found at nucleotide positions 598-600 (FIG. 51; SEQ ID NO:51). The predicted polypeptide precursor (FIG. 52, SEQ ID NO:52) is 198 amino acids long. The signal peptide is at about amino acids 1-21 of SEQ ID NO:52. PRO1185 has a calculated molecular weight of approximately 22,105 daltons and an estimated pI of approximately 7.73. Clone DNA62881-1515 has been deposited with the ATCC (Aug. 4, 1998) and is assigned ATCC deposit no. 203096.

An analysis of the Dayhoff database (version 35.45 SwissProt 35), using a WU-BLAST2 sequence alignment analysis of the full-length sequence shown in FIG. 52 (SEQ ID NO:52), revealed some sequence identity between the PRO1185 amino acid sequence and the following Dayhoff sequences: TUP1_YEAST, AF041382_{—}1, MAOM_SOLTU, SPPBPHU9_{—}1, I41024, EPCPLCFAIL_{—}1, HSPLEC_{—}1, YKL4 CAEEL, A44643, TGU65922_{—}1.

### Example 29

### Isolation of cDNA Clones Encoding Human PRO1194 Polypeptides [UNQ607]

Use of the signal sequence algorithm described in Example 3 above allowed identification of an EST cluster sequence from the Incyte database. This EST cluster sequence was then compared to a variety of expressed sequence tag (EST) databases which included public EST databases (e.g., GenBank) and a proprietary EST DNA database (LIFESEQ®, Incyte Pharmaceuticals, Palo Alto, Calif.) to identify existing homologies. One or more of the ESTs was derived from a human pineal gland library. The homology search was performed using the computer program BLAST or BLAST2 (Altshul et al., Methods in Enzymology 266:460-480 (1996)). Those comparisons resulting in a BLAST score of 70 (or in some cases 90) or greater that did not encode known proteins were clustered and assembled into a consensus DNA sequence with the program “phrap” (Phil Green, University of Washington, Seattle, Wash.). The consensus sequence obtained therefrom is herein designated DNA56511.

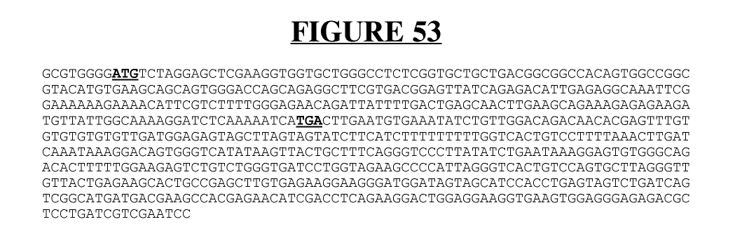

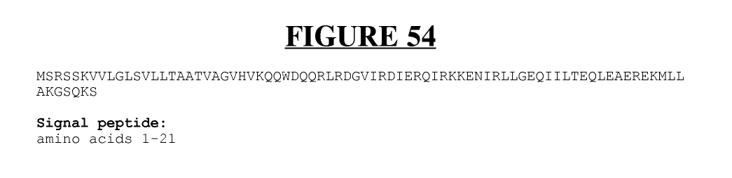

In light of the sequence homology between the DNA56511 sequence and an EST contained within the Merck EST AA069568, the clone 382736 which includes this EST was purchased and the cDNA insert was obtained and sequenced. The sequence of this cDNA insert is shown in FIG. 53 and is herein designated as DNA57841-1522.

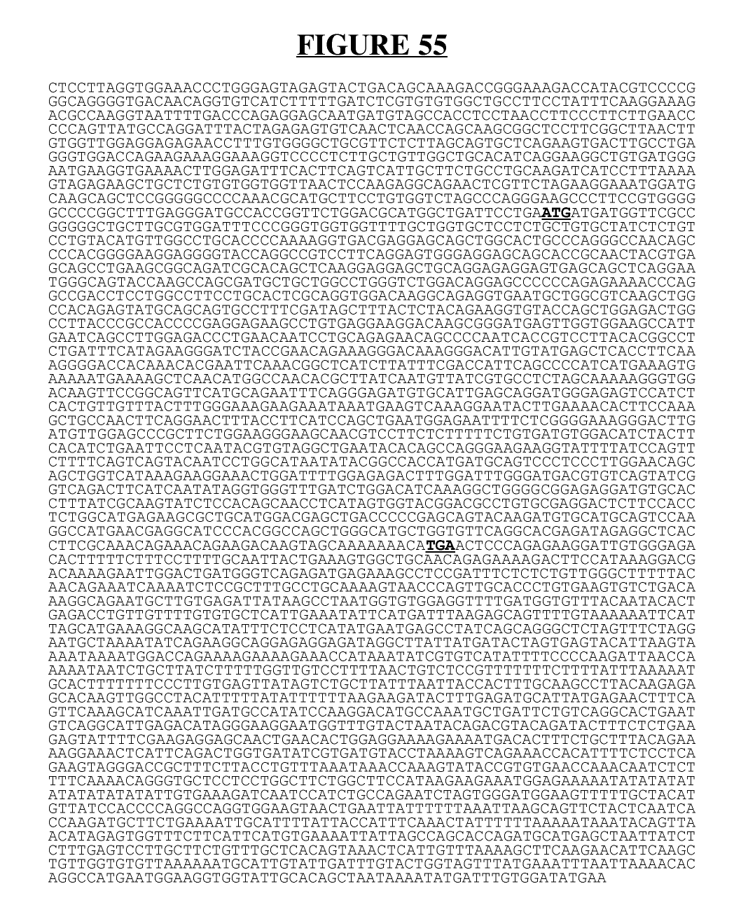

The full length clone shown in FIG. 53 contained a single open reading frame with an apparent translational initiation site at nucleotide positions 9-11 and ending at the stop codon found at nucleotide positions 252-254 (FIG. 53; SEQ ID NO:53). The predicted polypeptide precursor (FIG. 54, SEQ ID NO:54) is 81 amino acids long. The signal peptide is at about amino acids 1-21 of SEQ ID NO:54. PRO1194 has a calculated molecular weight of approximately 9,223 daltons and an estimated pI of approximately 10.47. Clone DNA57841-1522 was deposited with the ATCC on Nov. 3, 1998 and is assigned ATCC deposit no. 203458.

An analysis of the Dayhoff database (version 35.45 SwissProt 35), using a WU-BLAST2 sequence alignment analysis of the full-length sequence shown in FIG. 54 (SEQ ID NO:54), revealed sequence identity between the PRO1194 amino acid sequence and the following Dayhoff sequences: PT17_YEAST, RR2_CHLVU, CEK12F2_{—}1, S22452, S76705, AF031898_{—}7, A4_DROME, AF038931_{—}1, E49905, and GSPL_AERHY.

### Example 30

### Isolation of cDNA Clones Encoding Human PRO1287 Polypeptides [UNQ656]

An expressed sequence tag (EST) DNA database (LIFESEQ®, Incyte Pharmaceuticals, Palo Alto, Calif.) was searched and an EST was identified which showed homology to the fringe protein. This EST sequence was then compared to various EST databases including public EST databases (e.g., GenBank), and a proprietary EST database (LIFESEQ®, Incyte Pharmaceuticals, Palo Alto, Calif.) to identify homologous EST sequences. The comparison was performed using the computer program BLAST or BLAST2 [Altschul et al., Methods in Enzymology, 266:460-480 (1996)]. Those comparisons resulting in a BLAST score of 70 (or in some cases, 90) or greater that did not encode known proteins were clustered and assembled into a consensus DNA sequence with the program “phrap” (Phil Green, University of Washington, Seattle, Wash.). This consensus sequence obtained is herein designated DNA40568.

Based on the DNA40568 consensus sequence, oligonucleotides were synthesized: 1) to identify by PCR a cDNA library that contained the sequence of interest, and 2) for use as probes to isolate a clone of the full-length coding sequence for PRO1287. Forward and reverse PCR primers generally range from 20 to 30 nucleotides and are often designed to give a PCR product of about 100-1000 by in length. The probe sequences are typically 40-55 by in length. In some cases, additional oligonucleotides are synthesized when the consensus sequence is greater than about 1-1.5 kbp. In order to screen several libraries for a full-length clone, DNA from the libraries was screened by PCR amplification, as per Ausubel et al., Current Protocols in Molecular Biology, supra, with the PCR primer pair. A positive library was then used to isolate clones encoding the gene of interest using the probe oligonucleotide and one of the primer pairs.

PCR primers (forward and reverse) were synthesized:

| | |
|---|---|
| forward PCR primer | |
| 5′-CTCGGGGAAAGGGACTTGATGTTGG-3′ | (SEQ ID NO: 215) |
| reverse PCR primer 1 | |
| 5′-GCGAAGGTGAGCCTCTATCTCGTGCC-3′ | (SEQ ID NO: 216) |
| reverse PCR primer 2 | |
| 5′-CAGCCTACACGTATTGAGG-3′ | (SEQ ID NO: 217) |

Additionally, a synthetic oligonucleotide hybridization probe was constructed from the consensus DNA40568 sequence which had the following nucleotide sequence

hybridization probe
(SEQ ID NO: 218)
5′-CAGTCAGTACAATCCTGGCATAATATACGGCCACCATGATGCAG
TCCC-3′.

In order to screen several libraries for a source of a full-length clone, DNA from the libraries was screened by PCR amplification with the PCR primer pairs identified above. A positive library was then used to isolate clones encoding the PRO1287 gene using the probe oligonucleotide and one of the PCR primers.

RNA for construction of the cDNA libraries was isolated from human bone marrow tissue. The cDNA libraries used to isolated the cDNA clones were constructed by standard methods using commercially available reagents such as those from Invitrogen, San Diego, Calif. The cDNA was primed with oligo dT containing a NotI site, linked with blunt to SalI hemikinased adaptors, cleaved with NotI, sized appropriately by gel electrophoresis, and cloned in a defined orientation into a suitable cloning vector (such as pRKB or pRKD; pRK5B is a precursor of pRK5D that does not contain the SfiI site; see, Holmes et al., Science, 253:1278-1280 (1991)) in the unique XhoI and NotI sites.

DNA sequencing of the clones isolated as described above gave the full-length DNA sequence for PRO1287 (designated herein as DNA61755-1554 [FIG. 55, SEQ ID NO:55]) and the derived protein sequence for PRO1287.

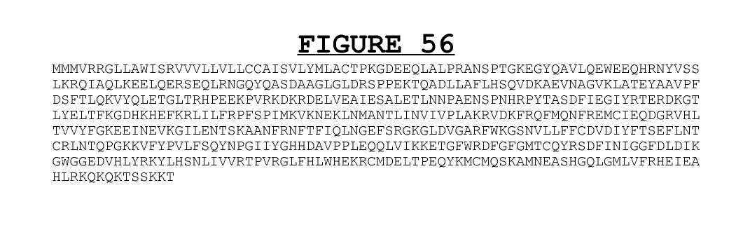

The entire nucleotide sequence of DNA61755-1554 is shown in FIG. 55 (SEQ ID NO:55). The full length clone contained a single open reading frame with an apparent translational initiation site at nucleotide positions 655-657 and a stop signal at nucleotide positions 2251-2253 (FIG. 55, SEQ ID NO:55). The predicted polypeptide precursor is 532 amino acids long, has a calculated molecular weight of approximately 61,351 daltons and an estimated pI of approximately 8.77. Analysis of the full-length PRO1287 sequence shown in FIG. 56 (SEQ ID NO:56) evidences the presence of the following: a signal peptide from about amino acid 1 to about amino acid 27 and potential N-glycosylation sites from about amino acid 315 to about amino acid 318 and from about amino acid 324 to about amino acid 327. Clone DNA61755-1554 has been deposited with ATCC on Aug. 11, 1998 and is assigned ATCC deposit no. 203112.

An analysis of the Dayhoff database (version 35.45 SwissProt 35), using a WU-BLAST2 sequence alignment analysis of the full-length sequence shown in FIG. 56 (SEQ ID NO:56), evidenced significant homology between the PRO1287 amino acid sequence and the following Dayhoff sequences: CET24D1_{—}1, EZRI_BOVIN, GGU19889_{—}1, CC3_YEAST, S74244, NALS_MOUSE, MOES_PIG, S28660, S44860 and YNA4_CAEEL.

### Example 31

### Isolation of cDNA Clones Encoding Human PRO1291 Polypeptides [UNQ659]

Use of the signal sequence algorithm described in Example 3 above allowed identification of an EST cluster sequence from the Incyte database, designated 120480. This EST cluster sequence was then compared to a variety of expressed sequence tag (EST) databases which included public EST databases (e.g., GenBank) and a proprietary EST DNA database (Lifeseq®, Incyte Pharmaceuticals, Palo Alto, Calif.) to identify existing homologies. The homology search was performed using the computer program BLAST or BLAST2 (Altshul et al., Methods in Enzymology 266:460-480 (1996)). Those comparisons resulting in a BLAST score of 70 (or in some cases 90) or greater that did not encode known proteins were clustered and assembled into a consensus DNA sequence with the program “phrap” (Phil Green, University of Washington, Seattle, Wash.). The consensus sequence obtained therefrom is herein designated DNA56425.

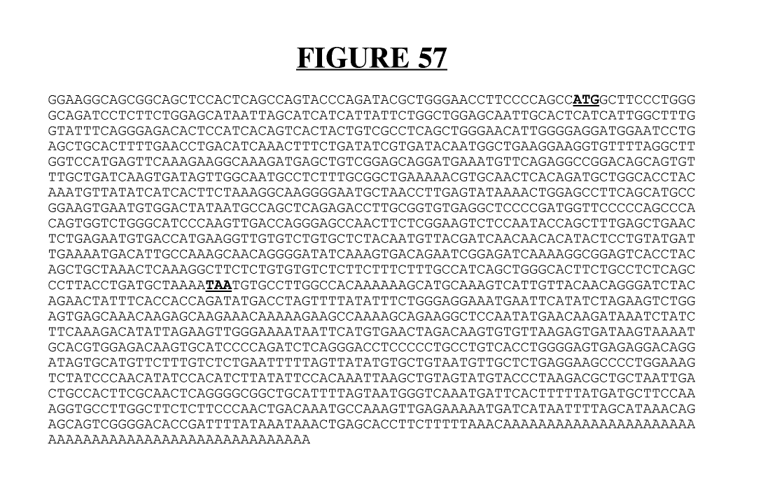

In light of an observed sequence homology between the DNA56425 sequence and an EST sequence encompassed within the Incyte EST clone no. 2798803, the Incyte EST clone 2798803 was purchased and the cDNA insert was obtained and sequenced. It was found that this insert encoded a full-length protein. The sequence of this cDNA insert is shown in FIG. 57 and is herein designated as DNA59610-1556.

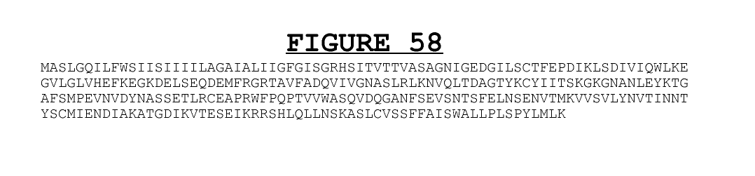

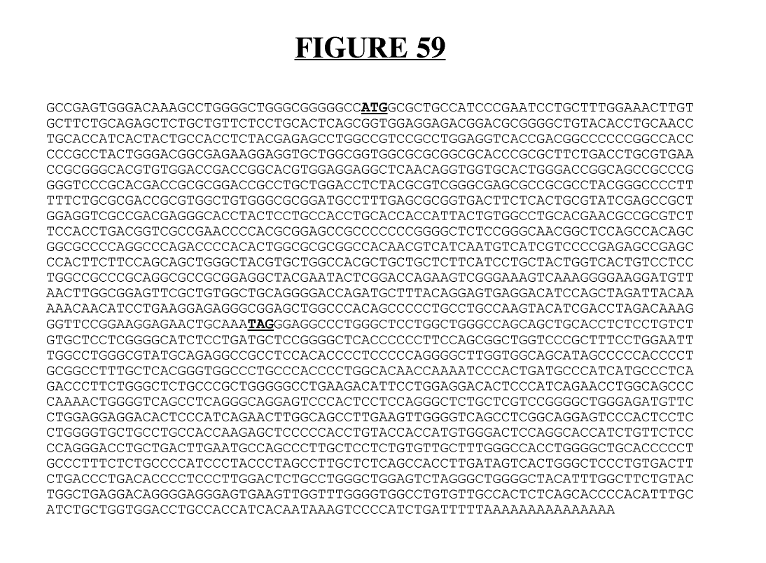

Clone DNA59610-1556 contains a single open reading frame with an apparent translational initiation site at nucleotide positions 61-63 and ending at the stop codon at nucleotide positions 907-909 (FIG. 57; SEQ ID NO:57). The predicted polypeptide precursor is 282 amino acids long (FIG. 58; SEQ ID NO:58). The full-length PRO1291 protein shown in FIG. 58 has an estimated molecular weight of about 30,878 daltons and a pI of about 5.27. Analysis of the full-length PRO1291 sequence shown in FIG. 58 (SEQ ID NO:58) evidences the presence of the following: a signal peptide from about amino acid 1 to about amino acid 28, a transmembrane domain from about amino acid 258 to about amino acid 281 and potential N-glycosylation sites from about amino acid 112 to about amino acid 115, from about amino acid 160 to about amino acid 163, from about amino acid 190 to about amino acid 193, from about amino acid 196 to about amino acid 199, from about amino acid 205 to about amino acid 208, from about amino acid 216 to about amino acid 219 and from about amino acid 220 to about amino acid 223. Clone DNA59610-1556 has been deposited with ATCC on Jun. 16, 1998 and is assigned ATCC deposit no. 209990.

An analysis of the Dayhoff database (version 35.45 SwissProt 35), using a WU-BLAST2 sequence alignment analysis of the full-length sequence shown in FIG. 58 (SEQ ID NO:58), evidenced significant homology between the PRO1291 amino acid sequence and the following Dayhoff sequences: HSU90552_{—}1, HSU90144_{—}1, AF033107_{—}1, HSB73_{—}1, HSU90142_{—}1, GGCD80_{—}1, P_W34452, MOG_MOUSE, B39371 and P_R71360.

### Example 32

### Isolation of cDNA Clones Encoding Human PRO1293 Polypeptides [UNQ662]

Use of the signal sequence algorithm described in Example 3 above allowed identification of an EST cluster sequence from the Incyte database, designated Incyte EST cluster sequence no. 115204. This EST cluster sequence was then compared to a variety of expressed sequence tag (EST) databases which included public EST databases (e.g., GenBank) and a proprietary EST DNA database (Lifeseq®, Incyte Pharmaceuticals, Palo Alto, Calif.) to identify existing homologies. The homology search was performed using the computer program BLAST or BLAST2 (Altshul et al., Methods in Enzymology 266:460-480 (1996)). Those comparisons resulting in a BLAST score of 70 (or in some cases 90) or greater that did not encode known proteins were clustered and assembled into a consensus DNA sequence with the program “phrap” (Phil Green, University of Washington, Seattle, Wash.). The consensus sequence obtained therefrom is herein designated DNA56522.

In light of the sequence homology between the DNA56522 sequence and an EST contained within the Incyte EST clone no. 2966119, the Incyte EST clone no. 2966119 was purchased and the cDNA insert was obtained and sequenced. The sequence of this cDNA insert is shown in FIG. 59 and is herein designated as DNA60618-1557.

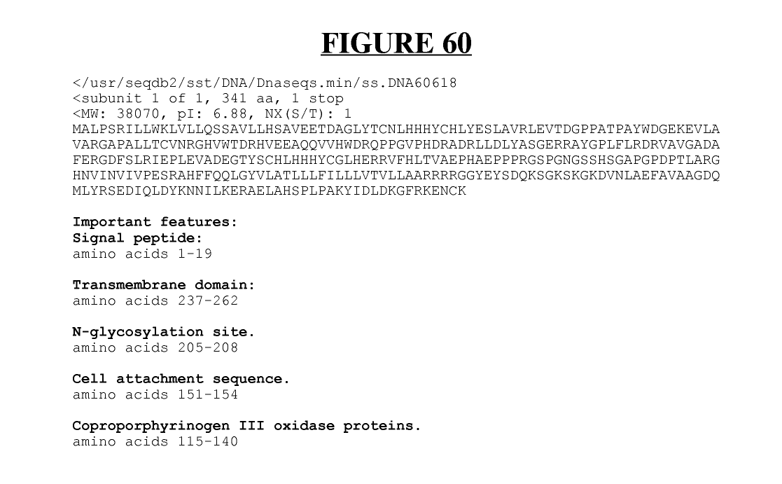

Clone DNA60618-1557 contains a single open reading frame with an apparent translational initiation site at nucleotide positions 37-39 and ending at the stop codon at nucleotide positions 1060-1062 (FIG. 59; SEQ ID NO:59). The predicted polypeptide precursor is 341 amino acids long (FIG. 60; SEQ ID NO:60). The full-length PRO1293 protein shown in FIG. 60 has an estimated molecular weight of about 38,070 daltons and a pI of about 6.88. Analysis of the full-length PRO1293 sequence shown in FIG. 60 (SEQ ID NO:60) evidences the presence of the following: a signal peptide from about amino acid 1 to about amino acid 19, a transmembrane domain from about amino acid 237 to about amino acid 262, a potential N-glycosylation site from about amino acid 205 to about amino acid 208, a cell attachment sequence from about amino acid 151 to about amino acid 152 and an amino acid sequence block having homology to coproporphyrinogen III oxidase proteins from about amino acid 115 to about amino acid 140. Clone DNA60618-1557 has been deposited with ATCC on Sep. 29, 1998 and is assigned ATCC deposit no. 203292.

An analysis of the Dayhoff database (version 35.45 SwissProt 35), using a WU-BLAST2 sequence alignment analysis of the full-length sequence shown in FIG. 60 (SEQ ID NO:60), evidenced significant homology between the PRO1293 amino acid sequence and the following Dayhoff sequences: HSVCD54_{—}1, A33_HUMAN, AF009220_{—}1, HSU82279_{—}1, AF004230_{—}1, P_R13272, AF004231_{—}1, AF043644_{—}1, S44125 and HSIGGHC85_{—}1.

### Example 33

### Isolation of cDNA Clones Encoding Human PRO1310 Polypeptides [UNQ676]

A consensus DNA sequence was assembled relative to other EST sequences using phrap as described in Example 1 above. This consensus sequence is designated herein as DNA37164. Based on the DNA37164 consensus sequence, oligonucleotides were synthesized: 1) to identify by PCR a cDNA library that contained the sequence of interest, and 2) for use as probes to isolate a clone of the full-length coding sequence for PRO1310.

PCR primers (forward and reverse) were synthesized:

| | |
|---|---|
| forward PCR primer: | |
| 5′GTTCTCAATGAGCTACCCGTCCCC3′ | (SEQ ID NO: 219) |
| and | |
| reverse PCR primer: | |
| 5′CGCGATGTAGTGGAACTCGGGCTC3′. | (SEQ ID NO: 220) |

Additionally, a synthetic oligonucleotide hybridization probe was constructed from the consensus DNA47394 sequence which had the following nucleotide sequence:

hybridization probe:
(SEQ ID NO: 221)
5′ATCCGCATAAACCCTCAGTCCTGGTTTGATAATGGGAGCATCTGCA
TGAG3′.

In order to screen several libraries for a source of a full-length clone, DNA from the libraries was screened by PCR amplification with the PCR primer pair identified above. A positive library was then used to isolate clones encoding the PRO1310 gene using the probe oligonucleotide and one of the PCR primers. RNA for construction of the cDNA libraries was isolated from human fetal liver tissue.

DNA sequencing of the clones isolated as described above gave the full-length DNA sequence for PRO1310 and the derived protein sequence for PRO1310.

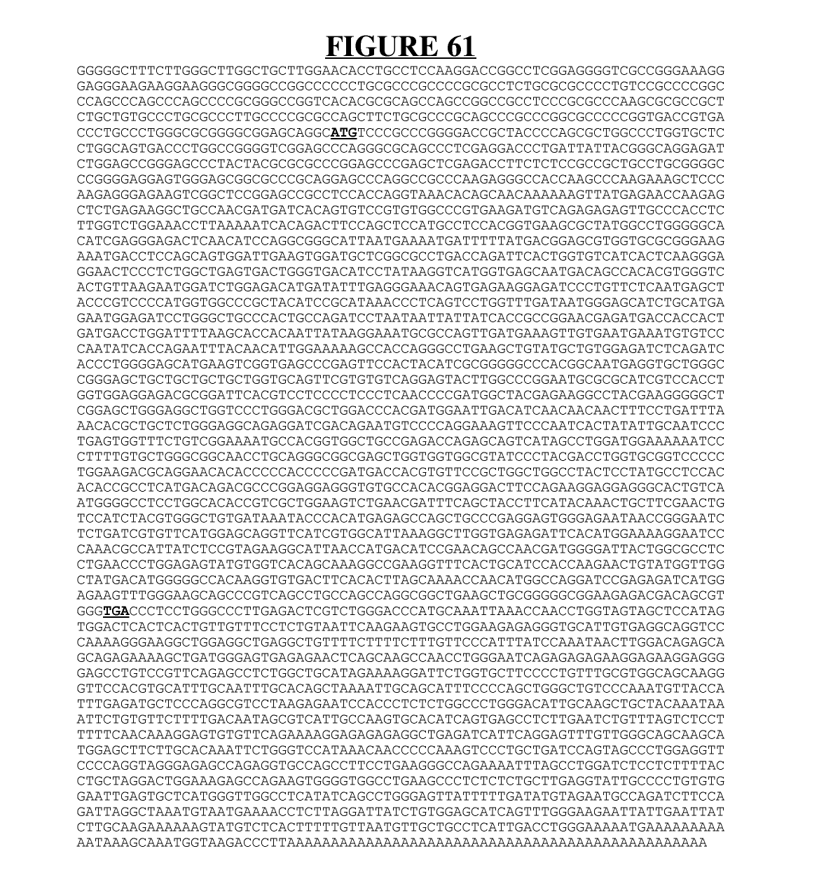

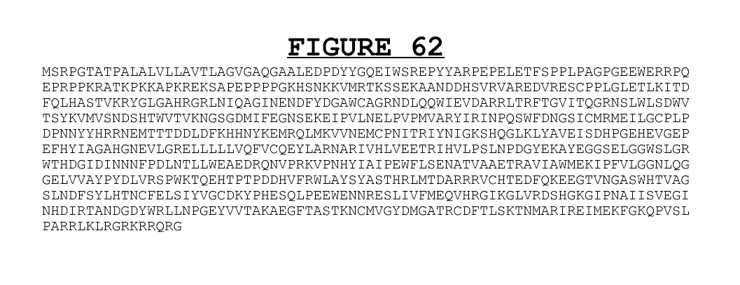

The entire coding sequence of PRO1310 is shown in FIG. 61 (SEQ ID NO:61). Clone DNA47394-1572 contains a single open reading frame with an apparent translational initiation site at nucleotide positions 326-328, and an apparent stop codon at nucleotide positions 2594-2596 (SEQ ID NO:61). The predicted polypeptide precursor is 765 amino acids long. The signal peptide is at about amino acids 1-25 of SEQ ID NO:62. Clone DNA47394-1572 has been deposited with ATCC (Aug. 11, 1998) and is assigned ATCC deposit no. 203109. The full-length PRO1310 protein shown in FIG. 62 has an estimated molecular weight of about 85,898 daltons and a pI of about 6.87.

An analysis of the Dayhoff database (version 35.45 SwissProt 35), using a WU-BLAST-2 sequence alignment analysis of the full-length sequence shown in FIG. 62 (SEQ ID NO:62), revealed sequence identity between the PRO1310 amino acid sequence and the following Dayhoff sequences: AF017639_{—}1, P_W36817, JC5256, CBPH_HUMAN, MMU23184_{—}1, CBPN_HUMAN, HSU83411_{—}1, CEF01D4_{—}7, RNU62897_{—}1 and P_W11851.

### Example 34

### Isolation of cDNA Clones Encoding Human PRO1312 Polypeptides [UNQ678]

DNA55773 was identified in a human fetal kidney cDNA library using a yeast screen that preferentially represents the 5′ ends of the primary cDNA clones. Based on the DNA55773 sequence, oligonucleotides were synthesized for use as probes to isolate a clone of the full-length coding sequence for PRO1312.

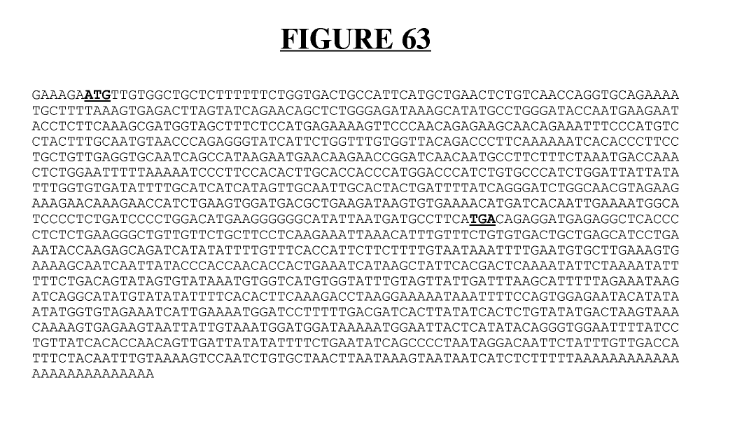

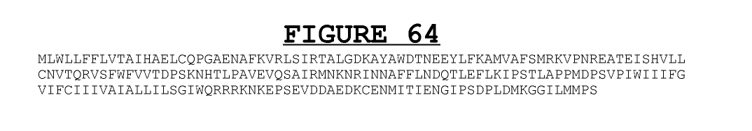

The full length DNA61873-1574 clone shown in FIG. 63 (SEQ ID NO:63) contained a single open reading frame with an apparent translational initiation site at nucleotide positions 7-9 and ending at the stop codon found at nucleotide positions 643-645. The predicted polypeptide precursor is 212 amino acids long (FIG. 64, SEQ ID NO:64). PRO1312 has a calculated molecular weight of approximately 24,024 daltons and an estimated pI of approximately 6.26. Other features include a signal peptide at about amino acids 1-14; a transmembrane domain at about amino acids 141-160, and potential N-glycosylation sites at about amino acids 76-79 and 93-96.

An analysis of the Dayhoff database (version 35.45 SwissProt 35), using a WU-BLAST2 sequence alignment analysis of the full-length sequence shown in FIG. 64 (SEQ ID NO:64), revealed some homology between the PRO1312 amino acid sequence and the following Dayhoff sequences: GCINTALPH_{—}1, GIBMUC1A_{—}1, P_R96298, AF001406_{—}1, PVU88874_{—}1, P_R85151, AF041409_{—}1, CELC50F2_{—}7, C45875, and AB009510_{—}21.

Clone DNA61873-1574 has been deposited with ATCC (Aug. 18, 1998) and is assigned ATCC deposit no. 203132.

### Example 35

### Isolation of cDNA Clones Encoding Human PRO1335 Polypeptides [UNQ690]

A consensus DNA sequence was assembled relative to other EST sequences using phrap as described in Example 1 above. This consensus sequence is herein designated DNA35727. Based on the DNA35727 consensus sequence, oligonucleotides were synthesized: 1) to identify by PCR a cDNA library that contained the sequence of interest, and 2) for use as probes to isolate a clone of the full-length coding sequence for PRO1335.

PCR primers (forward and reverse) were synthesized:

| | |
|---|---|
| forward PCR primer (35727.f1) | |
| 5′-GTAAAGTCGCTGGCCAGC-3′ | (SEQ ID NO: 222) |
| forward PCR primer (35727.f2) | |
| 5′-CCCGATCTGCCTGCTGTA-3′ | (SEQ ID NO: 223) |
| reverse PCR primer (35727.r1) | |
| 5′-CTGCACTGTATGGCCATTATTGTG-3′ | (SEQ ID NO: 224) |

Additionally, a synthetic oligonucleotide hybridization probe was constructed from the consensus DNA35727 sequence which had the following nucleotide sequence

hybridization probe (35727.p1)
(SEQ ID NO: 225)
5′-CAGAAACCCATGATACCCTACTGAACACCGAATCCCCTGGAAGCC-3′

In order to screen several libraries for a source of a full-length clone, DNA from the libraries was screened by PCR amplification with the PCR primer pair identified above. A positive library was then used to isolate clones encoding the PRO1335 gene using the probe oligonucleotide and one of the PCR primers. RNA for construction of the cDNA libraries was isolated from human retina tissue.

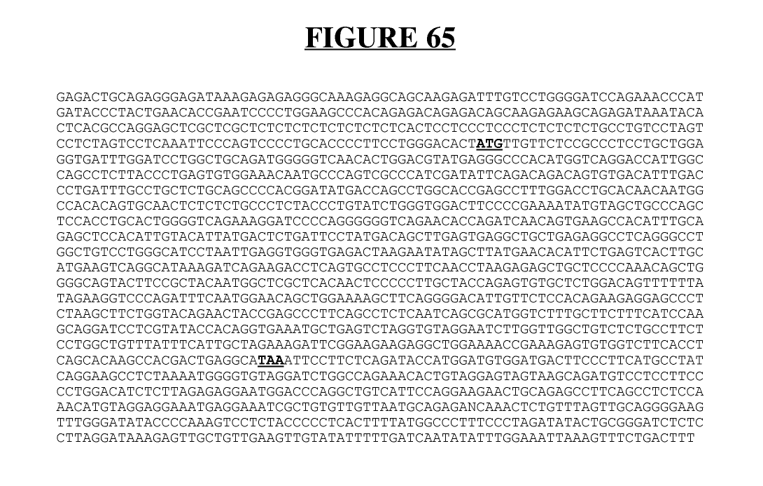

DNA sequencing of the clones isolated as described above gave the full-length DNA sequence for PRO1335 (designated herein as DNA62812-1594 [FIG. 65, SEQ ID NO:65]; and the derived protein sequence for PRO1335.

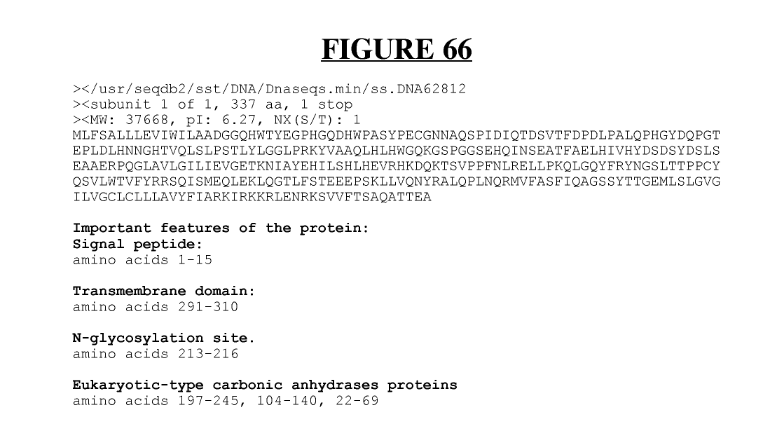

The entire nucleotide sequence of DNA62812-1594 is shown in FIG. 65 (SEQ ID NO:65). Clone DNA62812-1594 contains a single open reading frame with an apparent translational initiation site at nucleotide positions 271-273 and ending at the stop codon at nucleotide positions 1282-1284 (FIG. 65). The predicted polypeptide precursor is 337 amino acids long (FIG. 66; SEQ ID NO:66). The full-length PRO1335 protein shown in FIG. 66 has an estimated molecular weight of about 37,668 daltons and a pI of about 6.27. Analysis of the full-length PRO1335 sequence shown in FIG. 66 (SEQ ID NO:66) evidences the presence of the following: a signal peptide from about amino acid 1 to about amino acid 15, a transmembrane domain from about amino acid 291 to about amino acid 310, a potential N-glycosylation site from about amino acid 213 to about amino acid 216 and amino acid sequence blocks having homology to eukaryotic-type carbonic anhydrase proteins from about amino acid 197 to about amino acid 245, from about amino acid 104 to about amino acid 140 and from about amino acid 22 to about amino acid 69. Clone DNA62812-1594 has been deposited with ATCC on Sep. 9, 1998 and is assigned ATCC deposit no. 203248.

An analysis of the Dayhoff database (version 35.45 SwissProt 35), using a WU-BLAST2 sequence alignment analysis of the full-length sequence shown in FIG. 66 (SEQ ID NO:66), evidenced significant homology between the PRO1335 amino acid sequence and the following Dayhoff sequences: AF037335_{—}1, I38013, PTPG_MOUSE, CAH2_HUMAN, 1CAC, CAH7_HUMAN, CAH3_HUMAN, CAH1 HUMAN, CAH5_HUMAN and P_R41746.

### Example 36

### Isolation of cDNA Clones Encoding Human PRO1339 Polypeptides [UNQ694]

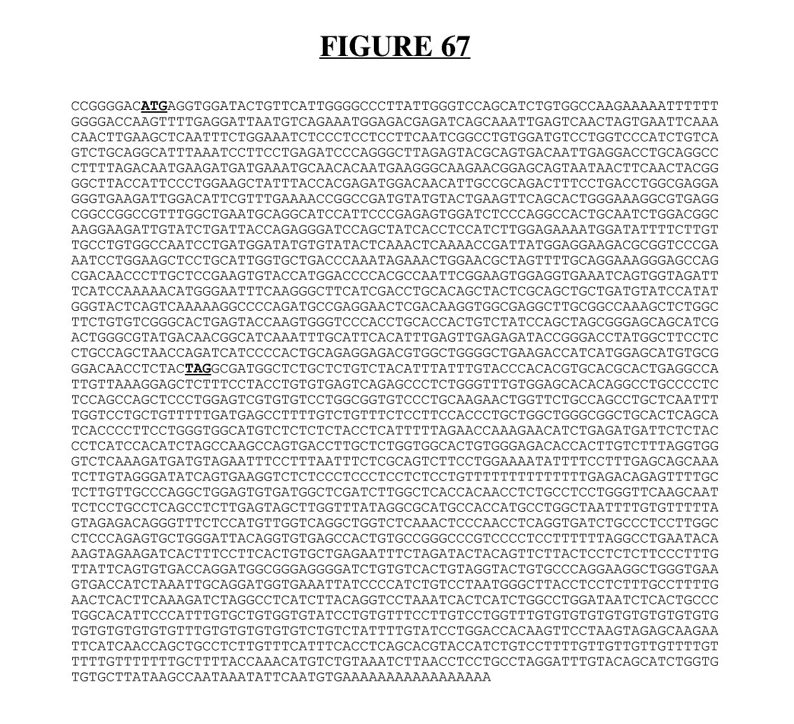

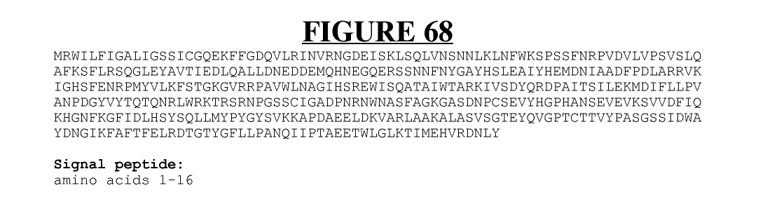

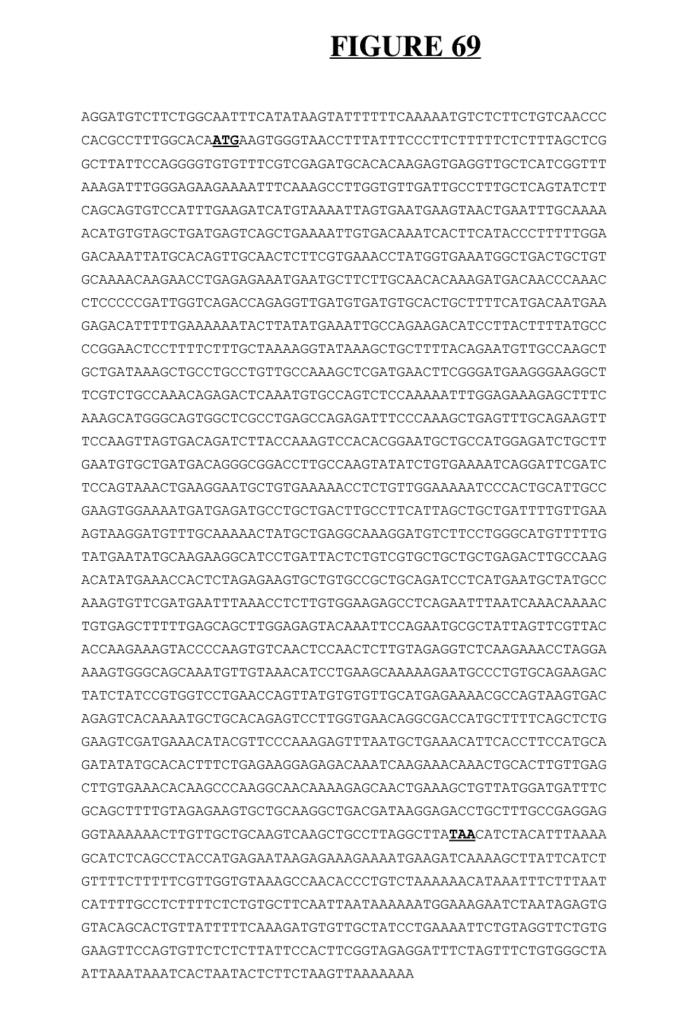

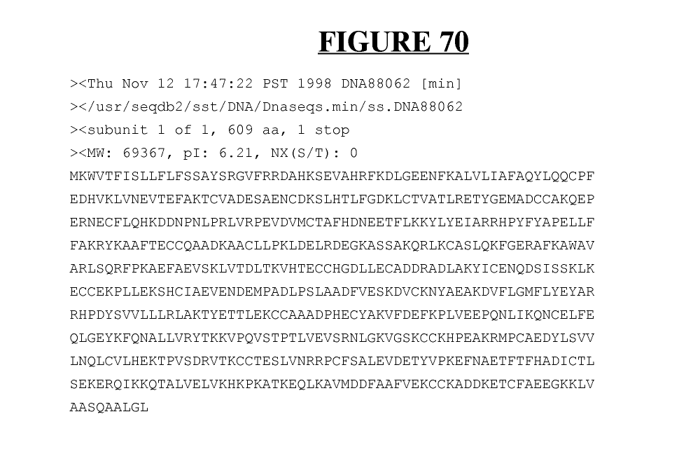

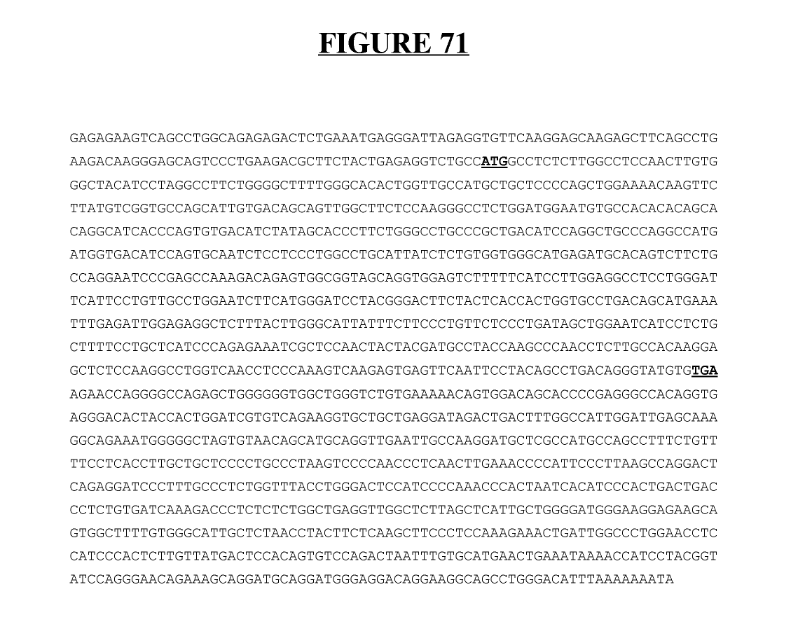

A consensus DNA sequence was assembled relative to other EST sequences using phrap as described in Example 1 above. This consensus sequence is designated herein “DNA40652”. Within the consensus sequence assembly was Incyte EST 2479394. Based on the consensus sequence and other discoveries and information provided herein, the clone including Incyte EST 2479394 was purchased and sequenced in full. Sequencing provided the nucleic acid sequence shown in FIG. 67 which includes the sequence encoding PRO1339.

Clone DNA66669-1597 contains a single open reading frame with an apparent translational initiation site at nucleotide positions 9-11 and an apparent stop codon at nucleotide positions 1272-1274 of FIG. 67 (SEQ ID NO:67). The predicted polypeptide precursor is 421 amino acids long. The signal peptide is at about amino acids 1-16 of FIG. 68 (SEQ ID NO:68). The region conserved in zinc carboxypeptidases and the N-glycosylation site are indcated in FIG. 68. Clone DNA66669-1597 has been deposited with the ATCC (Sep. 22, 1998) and is assigned ATCC deposit no. 203272. The full-length PRO1339 protein shown in FIG. 68 has an estimated molecular weight of about 47,351 daltons and a pI of about 6.61.

An analysis of the Dayhoff database (version 35.45 SwissProt 35), using a WU-BLAST2 sequence alignment analysis of the full-length sequence shown in FIG. 68 (SEQ ID NO:68), revealed sequence identity between the PRO1339 amino acid sequence and the following Dayhoff sequences (data incorporated herein): P_WO1505, CBP1_HUMAN, HSA224866_{—}1, P_R90293, YHT2 YEAST, CEF02D8_{—}4, CEW01A8_{—}6, P_W36815, HSU83411_{—}1 and CBPN_HUMAN.

### Example 37

### Isolation of cDNA Clones Encoding Human PRO1356 Polypeptides [UNQ705]

Use of the signal sequence algorithm described in Example 3 above allowed identification of an EST cluster sequence from the Incyte database, designated Incyte EST cluster sequence no. 44725. This EST cluster sequence was then compared to a variety of expressed sequence tag (EST) databases which included public EST databases (e.g., GenBank) and a proprietary EST DNA database (Lifeseq®, Incyte Pharmaceuticals, Palo Alto, Calif.) to identify existing homologies. The homology search was performed using the computer program BLAST or BLAST2 (Altshul et al., Methods in Enzymology 266:460-480 (1996)). Those comparisons resulting in a BLAST score of 70 (or in some cases 90) or greater that did not encode known proteins were clustered and assembled into a consensus DNA sequence with the program “phrap” (Phil Green, University of Washington, Seattle, Wash.). The consensus sequence obtained therefrom is herein designated DNA56023.

In light of the sequence homology between the DNA56023 sequence and an EST sequence contained within the Incyte EST clone no. 4071746, the Incyte EST clone no. 4071746 was purchased and the cDNA insert was obtained and sequenced. The sequence of this cDNA insert is shown in FIG. 71 and is herein designated as DNA64886-1601.

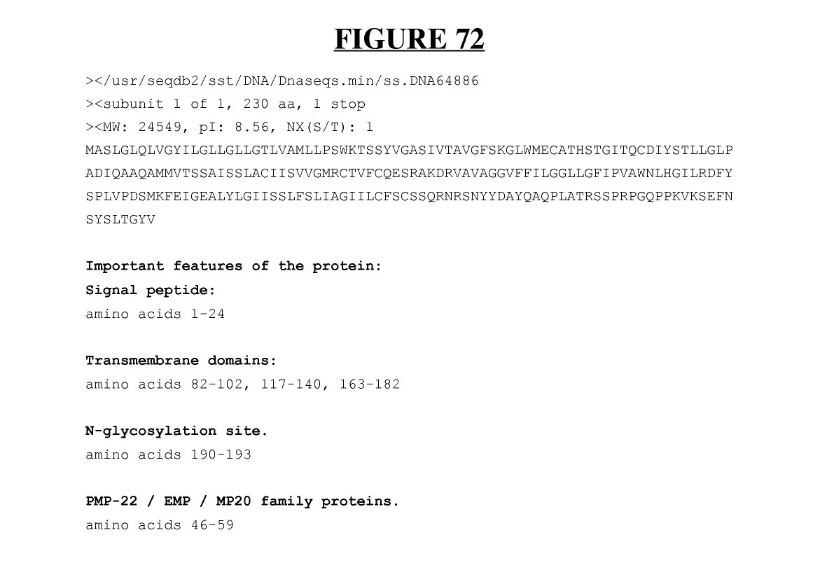

Clone DNA64886-1601 contains a single open reading frame with an apparent translational initiation site at nucleotide positions 122-124 and ending at the stop codon at nucleotide positions 812-814 (FIG. 71; SEQ ID NO:71). The predicted polypeptide precursor is 230 amino acids long (FIG. 72; SEQ ID NO:72). The full-length PRO1356 protein shown in FIG. 72 has an estimated molecular weight of about 24,549 daltons and a pI of about 8.56. Analysis of the full-length PRO1356 sequence shown in FIG. 72 (SEQ ID NO:72) evidences the presence of the following: a signal peptide from about amino acid 1 to about amino acid 24, transmembrane domains from about amino acid 82 to about amino acid 102, from about amino acid 117 to about amino acid 140 and from about amino acid 163 to about amino acid 182, a potential N-glycosylation site from about amino acid 190 to about amino acid 193 and an amino acid sequence block having homology to the PMP-22/EMP/MP20 family of proteins from about amino acid 46 to about amino acid 59. Clone DNA64886-1601 has been deposited with ATCC on Sep. 9, 1998 and is assigned ATCC deposit no. 203241. An analysis of the Dayhoff database (version 35.45 SwissProt 35), using a WU-BLAST2 sequence alignment analysis of the full-length sequence shown in FIG. 72 (SEQ ID NO:72), evidenced significant homology between the PRO1356 amino acid sequence and the following Dayhoff sequences: AB00014_{—}1, AB000712_{—}1, A39484, AF000959_{—}1, AF035814_{—}1, H5U89916_{—}1, MMU19582_{—}1, P_R30059, HUAC004125_{—}1 and PM22_RAT.

### Example 38

### Isolation of cDNA Clones Encoding Human PRO1385 Polypeptides [UNQ720]

Use of the signal sequence algorithm described in Example 3 above allowed identification of a single EST cluster sequence from the Incyte database. This EST cluster sequence was then compared to a variety of expressed sequence tag (EST) databases which included public EST databases (e.g., GenBank) and a proprietary EST DNA database (LIFESEQ®, Incyte Pharmaceuticals, Palo Alto, Calif.) to identify existing homologies. The homology search was performed using the computer program BLAST or BLAST2 (Altshul et al., Methods in Enzymology 266:460-480 (1996)). Those comparisons resulting in a BLAST score of 70 (or in some cases 90) or greater that did not encode known proteins were clustered and assembled into a consensus DNA sequence with the program “phrap” (Phil Green, University of Washington, Seattle, Wash.). The consensus sequence obtained therefrom is herein designated DNA57952.

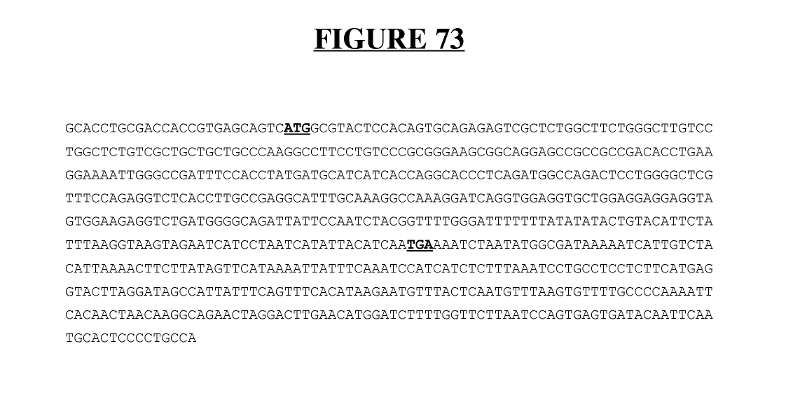

In light of an observed sequence homology between the DNA57952 consensus sequence and an EST sequence encompassed within the Incyte EST clone no. 3129630, the Incyte EST clone 3129630 was purchased and the cDNA insert was obtained and sequenced. It was found that this insert encoded a full-length protein. The sequence of this cDNA insert is shown in FIG. 73 and is herein designated as DNA68869-1610.

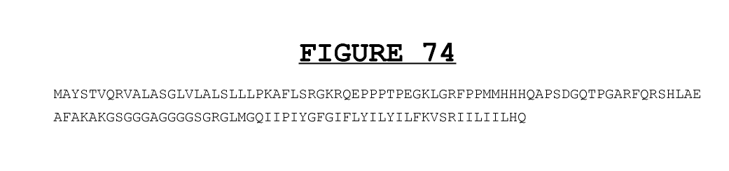

Clone DNA68869-1610 contains a single open reading frame with an apparent translational initiation site at nucleotide positions 26-28 and ending at the stop codon at nucleotide positions 410-412 (FIG. 73; SEQ ID NO:73). The predicted polypeptide precursor is 128 amino acids long (FIG. 74; SEQ ID NO:74). The full-length PRO1385 protein shown in FIG. 74 has an estimated molecular weight of about 13,663 daltons and a pI of about 10.97. Analysis of the full-length PRO1385 sequence shown in FIG. 74 (SEQ ID NO:74) evidences the presence of the following: a signal peptide from about amino acid 1 to about amino acid 28, and glycosylaminoglycan attachment sites from about amino acid 82 to about amino acid 85 and from about amino acid 91 to about amino acid 94. Clone DNA68869-1610 has been deposited with ATCC on Aug. 25, 1998 and is assigned ATCC deposit no. 203164.

An analysis of the Dayhoff database (version 35.45 SwissProt 35), using a WU-BLAST2 sequence alignment analysis of the full-length sequence shown in FIG. 74 (SEQ ID NO:74), evidenced low homology between the PRO1385 amino acid sequence and the following Dayhoff sequences: CELT14A8_{—}1, LMNACHRA1_{—}1, HXD9_HUMAN, CHKCMLF_{—}1, HS5PP34_{—}2, DMDRING_{—}1, A37107_{—}1, MMLUNGENE_{—}1, PUM_DROME and DMU25117_{—}1.

### Example 39

### Isolation of cDNA Clones Encoding Human PRO1412 Polypeptides [UNQ730]

Use of the signal sequence algorithm described in Example 3 above allowed identification of an EST cluster sequence from the LIFESEQ® database, designated Incyte Cluster No. 101368, also referred herein as “DNA10643”. This EST cluster sequence was then compared to a variety of expressed sequence tag (EST) databases which included public EST databases (e.g., GenBank) and a proprietary EST DNA database (LIFESEQ®, Incyte Pharmaceuticals, Palo Alto, Calif.) to identify existing homologies. The homology search was performed using the computer program BLAST or BLAST2 (Altshul et al., Methods in Enzymology 266:460-480 (1996)). Those comparisons resulting in a BLAST score of 70 (or in some cases 90) or greater that did not encode known proteins were clustered and assembled into a consensus DNA sequence with the program “phrap” (Phil Green, University of Washington, Seattle, Wash.). One or more of the ESTs was derived from RNA isolated from fibroblasts of the prostate stroma removed from a male fetus. The consensus sequence obtained therefrom is herein designated “DNA58754”.

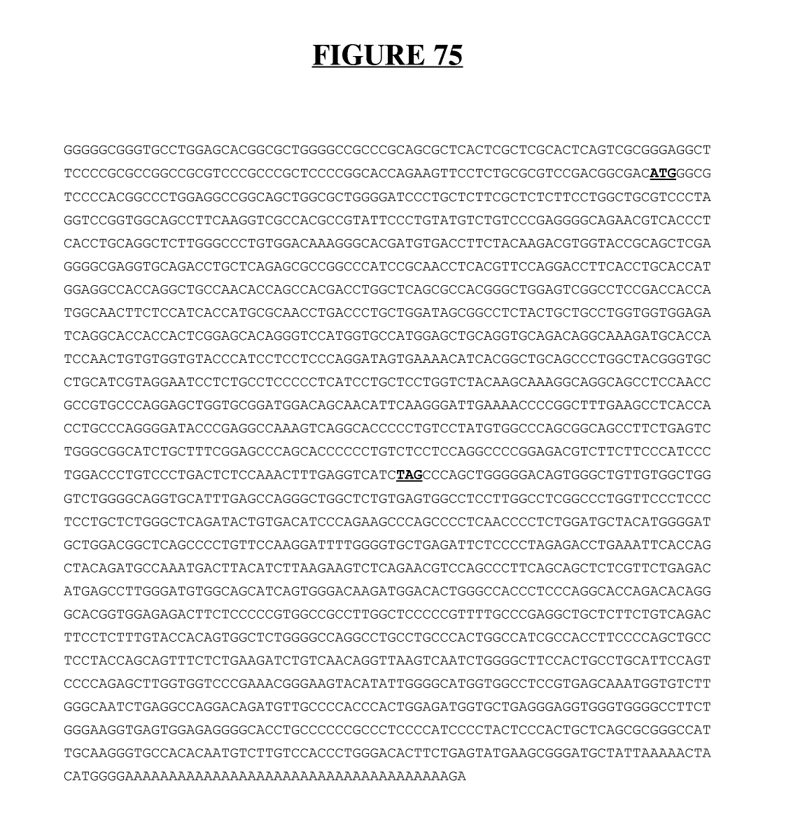

In light of the sequence homology between the DNA58754 sequence and an EST sequence contained within EST no. 3597385, the EST clone 3597385 was purchased and the cDNA insert was obtained and sequenced in its entirety. The sequence of this cDNA insert is shown in FIG. 75 and is herein designated as “DNA64897-1628”.

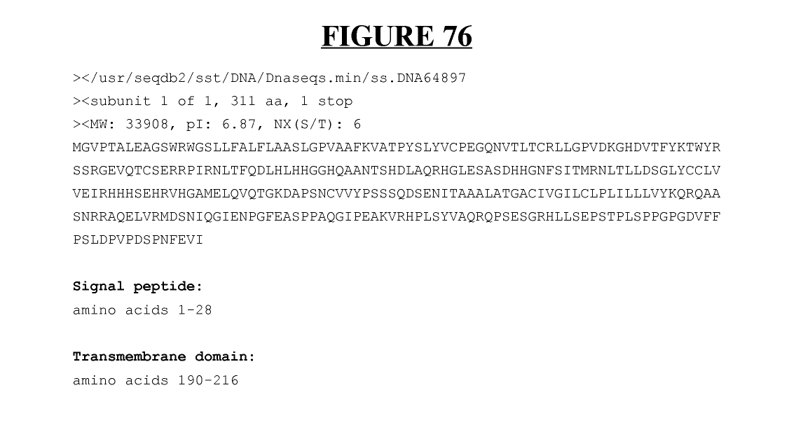

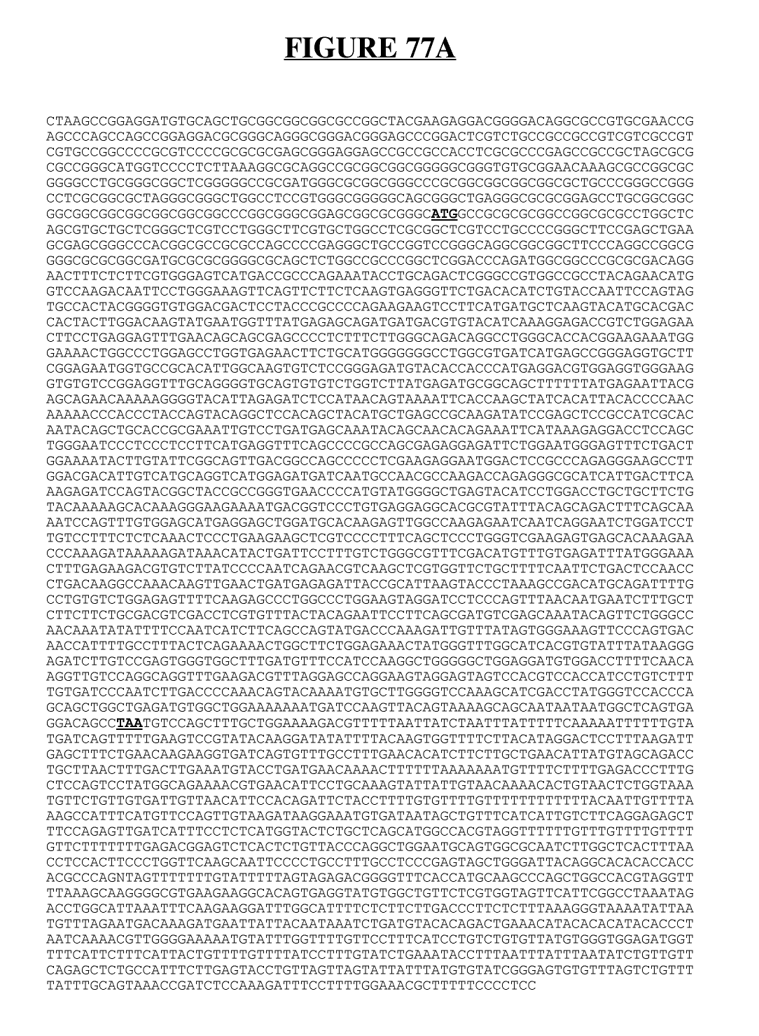

The full length clone shown in FIG. 75 contained a single open reading frame with an apparent translational initiation site at nucleotide positions 142 to 144 and ending at the stop codon found at nucleotide positions 1075 to 1077 (FIG. 75; SEQ ID NO:75). The predicted polypeptide precursor (FIG. 76, SEQ ID NO:76) is 311 amino acids long. Other features of the PRO1412 protein include: a signal sequence at about amino acids 1-28; a transmembrane domain at about amino acids 190-216; potential N-glycosylation sites at about amino acids 49-52, 91-94, 108-111, 128-131, 135-138 and 190-193; a tyrosine kinase phosphorylation site at about amino acids 62-69; and a lysosome-associated membrane glycoprotein duplicated domain at about amino acids 183-224. PRO1412 has a calculated molecular weight of approximately 33,908 daltons and an estimated pI of approximately 6.87. Clone DNA64897-1628 was deposited with the ATCC on Sep. 15, 1998, and is assigned ATCC deposit no. 203216.

An analysis of the Dayhoff database (version 35.45 SwissProt 35), using a WU-BLAST2 sequence alignment analysis of the full-length sequence shown in FIG. 76 (SEQ ID NO:76), revealed some homology between the PRO1412 amino acid sequence and the following Dayhoff sequences: I50116, AF035963_{—}1, NCA2_RAT, I61783, P_W07682, MMHC135G15_{—}3, S21461, MMIGL2_{—}1, ONHIGMV9A_{—}1 and MMU70448_{—}1.

### Example 40

### Isolation of cDNA Clones Encoding Human PRO1487 Polypeptides [UNQ756]

A single Merck EST, HSC2ID011, referred herein as “DNA8208”, was identified as an EST of interest having a BLAST score of 70 or greater that did not encode a known protein as described in Example 1 above. The DNA8208 sequence was extended using repeated cycles of BLAST and the program “phrap” (Phil Green, University of Washington, Seattle, Wash.) to extend the sequence as far as possible using the sources of EST sequences discussed above. The resulting consensus sequence is designated herein as “DNA68836”. Based on the DNA68836 consensus sequence, oligonucleotides were synthesized: 1) to identify by PCR a cDNA library that contained the sequence of interest, and 2) for use as probes to isolate a clone of the full-length coding sequence for PRO1487.

PCR primers (forward and reverse) were synthesized:

| (54209.f1 ; SEQ ID NO: 226) | |
|---|---|
| forward PCR primer: | GTGCCACTACGGGGTGTGGACGAC |
| and | |
| (54209.r1; SEQ ID NO: 227) | |
| reverse PCR primer | TCCCATTTCTTCCGTGGTGCCCAG |

Additionally, a synthetic oligonucleotide hybridization probe was constructed from the consensus DNA68836 sequence which had the following nucleotide sequence:

hybridization probe
(54209.p1; SEQ ID NO: 228)
CCAGAAGAAGTCCTTCATGATGCTCAAGTACATGCACGACCACTAC

In order to screen several libraries for a source of a full-length clone, DNA from the libraries was screened by PCR amplification with the PCR primer pair identified above. A positive library was then used to isolate clones encoding the PRO1487 gene using the probe oligonucleotide and one of the PCR primers. RNA for construction of the cDNA libraries was isolated human fetal kidney tissue.

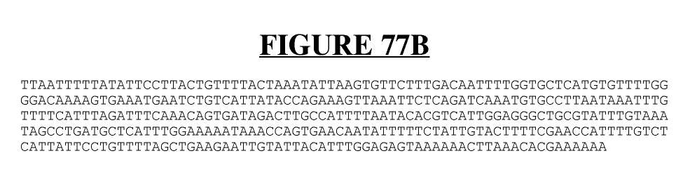

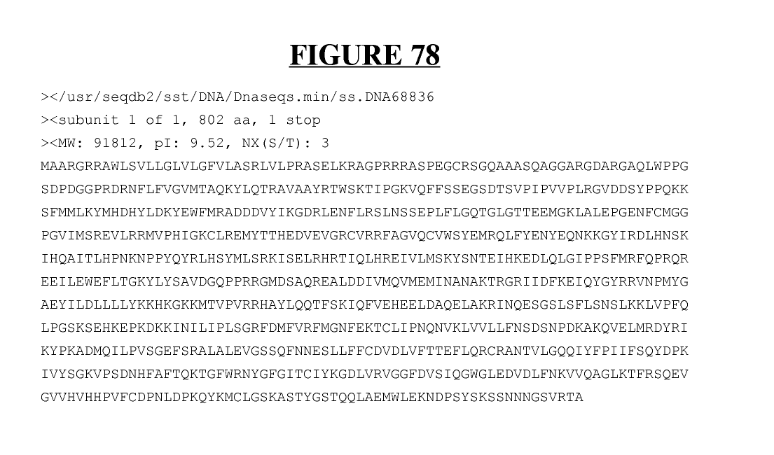

DNA sequencing of the clones isolated as described above gave the full-length DNA sequence for PRO1487 (designated herein as DNA68836-1656 (FIG. 77A-77B; SEQ ID NO:77) and the derived protein sequence for PRO1487 (FIG. 78; SEQ ID NO:78).

The entire coding sequence of PRO1487 is shown in FIG. 77A-77B (SEQ ID NO:77). Clone DNA68836-1656 contains a single open reading frame with an apparent translational initiation site at nucleotide positions 489-491 and an apparent stop codon at nucleotide positions 2895-2897. The predicted polypeptide precursor is 802 amino acids long The full-length PRO1487 protein shown in FIG. 78 has an estimated molecular weight of about 91,812 daltons and a pI of about 9.52. Additional features include a signal peptide at about amino acids 1-23; potential N-glycosylation sites at about amino acids 189-192, 623-626, and 796-799; and a cell attachment sequence at about amino acids 62-64.

An analysis of the Dayhoff database (version 35.45 SwissProt 35), using a WU-BLAST2 sequence alignment analysis of the full-length sequence shown in FIG. 78 (SEQ ID NO:78), revealed significant homology between the PRO1487 amino acid sequence and the following Dayhoff sequences: CET24D1_{—}1, S44860, CELC02H61, CEC38H2_{—}3, CELC17A2_{—}5, CETO9E11_{—}10, CEE03H4_{—}3, CELT22B11_{—}3, GGU82088_{—}1, and CEF56H6_{—}1.

Clone DNA68836-1656 was deposited with the ATCC on Nov. 3, 1998, and is assigned ATCC deposit no. 203455.

### Example 41

### Isolation of cDNA Clones Encoding Human PRO1758 Polypeptides [UNQ831]

Use of the signal sequence algorithm described in Example 3 above allowed identification of an EST cluster sequence from the LIFESEQ® database, designated EST cluster No. 20926. This EST cluster sequence was then compared to a variety of expressed sequence tag (EST) from the databases mentioned above, to identify existing homologies. The homology search was performed using the computer program BLAST or BLAST2 (Altshul et al., Methods in Enzymology 266:460-480 (1996)). Those comparisons resulting in a BLAST score of 70 (or in some cases 90) or greater that did not encode known proteins were clustered and assembled into a consensus DNA sequence with the program “phrap” (Phil Green, University of Washington, Seattle, Wash.). The consensus sequence obtained therefrom is herein designated DNA56260.

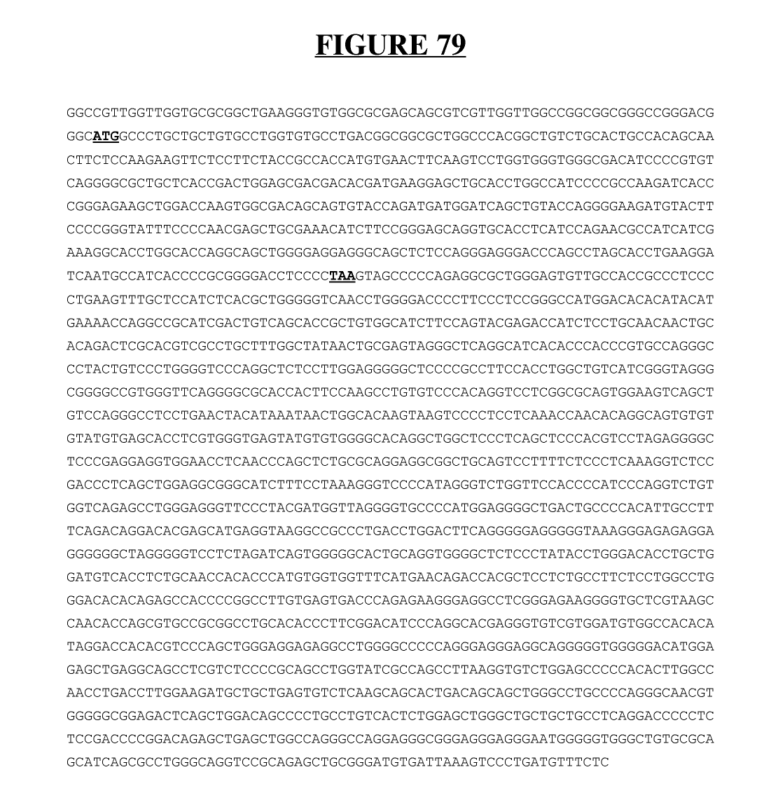

In light of the sequence homology between the DNA56260 sequence and a sequence contained within EST no. 2936330 from the LIFESEQ® database, the EST clone, which originated from a library constructed from thymus tissue of a fetus that died from anencephalus, was purchased and the cDNA insert was obtained and sequenced. The sequence of this cDNA insert is shown in FIG. 79 and is herein designated as DNA76399-1700.

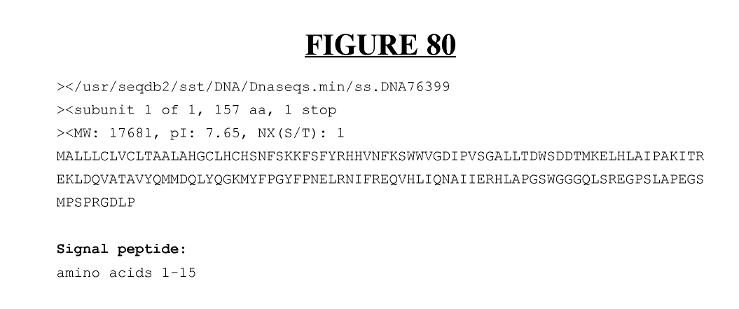

The full length clone shown in FIG. 79 contained a single open reading frame with an apparent translational initiation site at nucleotide positions 78 to 80 and ending at the stop codon found at nucleotide positions 549-551 (FIG. 79; SEQ ID NO:79). The predicted polypeptide precursor (FIG. 80, SEQ ID NO: 80) is 157 amino acids long. PRO1758 has a calculated molecular weight of approximately 17,681 daltons and an estimated pI of approximately 7.65. Additional features include: a signal peptide from about amino acids 1-15; a potential N-glycosylation site at about amino acids 24-27; a cAMP- and cGMP-dependent protein kinase phosphorylation site at about amino acids 27-30; a casein kinase II phosphorylation site at about amino acids 60-63; potential N-myristoylation sites at about amino acids 17-22, 50-55, 129-134, and 133-138; a cell attachment sequence at about amino acids 153-155; and a cytochrome c family heme-binding site signature at about amino acids 18-23.

An analysis of the Dayhoff database (version 35.45 SwissProt 35), using a WU-BLAST2 sequence alignment analysis of the full-length sequence shown in FIG. 80 (SEQ ID NO: 80), revealed significant homology between the PRO1758 amino acid sequence and Dayhoff sequence no AC0053282. Homology was also found between the PRO1758 amino acid sequence and Dayhoff sequence no. CELC46F2_{—}1.

Clone DNA76399-1700 was deposited with the ATCC on Nov. 17, 1998 and is assigned ATCC deposit no. 203472.

### Example 42

### Isolation of cDNA Clones Encoding Human PRO1779 Polypeptides [UNQ841]

1. Preparation of Oligo dT Primed cDNA Library

mRNA was isolated from human breast carcinoma tissue using reagents and protocols from Invitrogen, San Diego, Calif. (Fast Track 2). This RNA was used to generate an oligo dT primed cDNA library in the vector pRK5D using reagents and protocols from Life Technologies, Gaithersburg, Md. (Super Script Plasmid System). In this procedure, the double stranded cDNA was sized to greater than 1000 by and the SalI/NotI linkered cDNA was cloned into XhoI/NotI cleaved vector. pRK5D is a cloning vector that has an sp6 transcription initiation site followed by an SfiI restriction enzyme site preceding the XhoI/NotI cDNA cloning sites.

2. Preparation of Random Primed cDNA Library

A secondary cDNA library was generated in order to preferentially represent the 5′ ends of the primary cDNA clones. Sp6 RNA was generated from the primary library (described above), and this RNA was used to generate a random primed cDNA library in the vector pSST-AMY.0 using reagents and protocols from Life Technologies (Super Script Plasmid System, referenced above). In this procedure the double stranded cDNA was sized to 500-1000 bp, linkered with blunt to NotI adaptors, cleaved with SfiI, and cloned into SfiI/NotI cleaved vector. pSST-AMY.0 is a cloning vector that has a yeast alcohol dehydrogenase promoter preceding the cDNA cloning sites and the mouse amylase sequence (the mature sequence without the secretion signal) followed by the yeast alcohol dehydrogenase terminator, after the cloning sites. Thus, cDNAs cloned into this vector that are fused in frame with amylase sequence will lead to the secretion of amylase from appropriately transfected yeast colonies.

3. Transformation and Detection

DNA from the library described in paragraph 2 above was chilled on ice to which was added electrocompetent DH10B bacteria (Life Technologies, 20 ml). The bacteria and vector mixture was then electroporated as recommended by the manufacturer. Subsequently, SOC media (Life Technologies, 1 ml) was added and the mixture was incubated at 37° C. for 30 minutes. The transformants were then plated onto 20 standard 150 mm LB plates containing ampicillin and incubated for 16 hours (37° C.). Positive colonies were scraped off the plates and the DNA was isolated from the bacterial pellet using standard protocols, e.g. CsCl-gradient. The purified DNA was then carried on to the yeast protocols below.

The yeast methods were divided into three categories: (1) Transformation of yeast with the plasmid/cDNA combined vector; (2) Detection and isolation of yeast clones secreting amylase; and (3) PCR amplification of the insert directly from the yeast colony and purification of the DNA for sequencing and further analysis.

The yeast strain used was HD56-5A (ATCC-90785). This strain has the following genotype: MAT alpha, ura3-52, leu2-3, leu2-112, his3-11, his3-15, MAL⁺, SUC⁺, GAL⁺. Preferably, yeast mutants can be employed that have deficient post-translational pathways. Such mutants may have translocation deficient alleles in sec71, sec72, sec62, with truncated sec71 being most preferred. Alternatively, antagonists (including antisense nucleotides and/or ligands) which interfere with the normal operation of these genes, other proteins implicated in this post translation pathway (e.g., SEC61p, SEC72p, SEC62p, SEC63p, TDJ1p or SSA1p-4-p) or the complex formation of these proteins may also be preferably employed in combination with the amylase-expressing yeast.

Transformation was performed based on the protocol outlined by Gietz et al., Nucl. Acid. Res., 20:1425 (1992). Transformed cells were then inoculated from agar into YEPD complex media broth (100 ml) and grown overnight at 30° C. The YEPD broth was prepared as described in Kaiser et al., Methods in Yeast Genetics, Cold Spring Harbor Press, Cold Spring Harbor, N.Y., p. 207 (1994). The overnight culture was then diluted to about 2×10⁶ cells/ml (approx. OD₆₀₀=0.1) into fresh YEPD broth (500 ml) and regrown to 1×10⁷ cells/ml (approx. OD₆₀₀=0.4-0.5).

The cells were then harvested and prepared for transformation by transfer into GS3 rotor bottles in a Sorval GS3 rotor at 5,000 rpm for 5 minutes, the supernatant discarded, and then resuspended into sterile water, and centrifuged again in 50 ml falcon tubes at 3,500 rpm in a Beckman GS-6KR centrifuge. The supernatant was discarded and the cells were subsequently washed with LiAc/TE (10 ml, 10 mM Tris-HCl, 1 mM EDTA pH 7.5, 100 mM Li₂OOCCH₃), and resuspended into LiAc/TE (2.5 ml).

Transformation took place by mixing the prepared cells (100 μl) with freshly denatured single stranded salmon testes DNA (Lofstrand Labs, Gaithersburg, Md.) and transforming DNA (1 μg, vol. <10 μl) in microfuge tubes. The mixture was mixed briefly by vortexing, then 40% PEG/TE (600 μl, 40% polyethylene glycol-4000, 10 mM Tris-HCl, 1 mM EDTA, 100 mM Li₂OOCCH₃, pH 7.5) was added. This mixture was gently mixed and incubated at 30° C. while agitating for 30 minutes. The cells were then heat shocked at 42° C. for 15 minutes, and the reaction vessel centrifuged in a microfuge at 12,000 rpm for 5-10 seconds, decanted and resuspended into TE (500 μl, 10 mM Tris-HCl, 1 mM EDTA pH 7.5) followed by recentrifugation. The cells were then diluted into TE (1 ml) and aliquots (200 μl) were spread onto the selective media previously prepared in 150 mm growth plates (VWR).

Alternatively, instead of multiple small reactions, the transformation was performed using a single, large scale reaction, wherein reagent amounts were scaled up accordingly.

The selective media used was a synthetic complete dextrose agar lacking uracil (SCD-Ura) prepared as described in Kaiser et al., Methods in Yeast Genetics, Cold Spring Harbor Press, Cold Spring Harbor, N.Y., p. 208-210 (1994). Transformants were grown at 30° C. for 2-3 days.

The detection of colonies secreting amylase was performed by including red starch in the selective growth media. Starch was coupled to the red dye (Reactive Red-120, Sigma) as per the procedure described by Biely et al., Anal. Biochem., 172:176-179 (1988). The coupled starch was incorporated into the SCD-Ura agar plates at a final concentration of 0.15% (w/v), and was buffered with potassium phosphate to a pH of 7.0 (50-100 mM final concentration).

The positive colonies were picked and streaked across fresh selective media (onto 150 mm plates) in order to obtain well isolated and identifiable single colonies. Well isolated single colonies positive for amylase secretion were detected by direct incorporation of red starch into buffered SCD-Ura agar. Positive colonies were determined by their ability to break down starch resulting in a clear halo around the positive colony visualized directly.

4. Isolation of DNA by PCR Amplification

When a positive colony was isolated, a portion of it was picked by a toothpick and diluted into sterile water (30 μl) in a 96 well plate. At this time, the positive colonies were either frozen and stored for subsequent analysis or immediately amplified. An aliquot of cells (5 μl) was used as a template for the PCR reaction in a 25 μl volume containing: 0.5 μl Klentaq (Clontech, Palo Alto, Calif.); 4.0 μl 10 mM dNTP's (Perkin Elmer-Cetus); 2.5 μl Kentaq buffer (Clontech); 0.25 μl forward oligo 1; 0.25 μl reverse oligo 2; 12.5 μl distilled water. The sequence of the forward oligonucleotide 1 was:

(SEQ ID NO: 151)
5′-TGTAAAACGACGGCCAGTTAAATAGACCTGCAATTATTAATCT-3′

The sequence of reverse oligonucleotide 2 was:

(SEQ ID NO: 152)
5′-CAGGAAACAGCTATGACCACCTGCACACCTGCAAATCCATT-3′

PCR was then performed as follows:

| | | | | |
|---|---|---|---|---|
| a. | | Denature | 92° C., | 5 minutes |
| b. | 3 cycles of: | Denature | 92° C., | 30 seconds |
| | | Anneal | 59° C., | 30 seconds |
| | | Extend | 72° C., | 60 seconds |
| c. | 3 cycles of: | Denature | 92° C., | 30 seconds |
| | | Anneal | 57° C., | 30 seconds |
| | | Extend | 72° C., | 60 seconds |
| d. | 25 cycles of: | Denature | 92° C., | 30 seconds |
| | | Anneal | 55° C., | 30 seconds |
| | | Extend | 72° C., | 60 seconds |
| e. | | Hold | 4° C. | |

The underlined regions of the oligonucleotides annealed to the ADH promoter region and the amylase region, respectively, and amplified a 307 by region from vector pSST-AMY.0 when no insert was present. Typically, the first 18 nucleotides of the 5′ end of these oligonucleotides contained annealing sites for the sequencing primers. Thus, the total product of the PCR reaction from an empty vector was 343 bp. However, signal sequence-fused cDNA resulted in considerably longer nucleotide sequences.

Following the PCR, an aliquot of the reaction (5 μl) was examined by agarose gel electrophoresis in a 1% agarose gel using a Tris-Borate-EDTA (TBE) buffering system as described by Sambrook et al., supra. Clones resulting in a single strong PCR product larger than 400 by were further analyzed by DNA sequencing after purification with a 96 Qiaquick PCR clean-up column (Qiagen Inc., Chatsworth, Calif.).

5. Identification of Full-length Clone

A cDNA sequence isolated in the above screen is herein designated DNA66065. The DNA66065 sequence was then compared to a variety of expressed sequence tag (EST) databases which included public EST databases (e.g., GenBank), a proprietary EST DNA database (LIFESEQ®, Incyte Pharmaceuticals, Palo Alto, Calif.) and a proprietary Genentech EST database to identify existing homologies. The homology search was performed using the computer program BLAST or BLAST2 (Altshul et al., Methods in Enzymology 266:460-480 (1996)). Those comparisons resulting in a BLAST score of 70 (or in some cases 90) or greater that did not encode known proteins were clustered and assembled into consensus DNA sequences with the program “phrap” (Phil Green, University of Washington). The consensus sequence is herein designated DNA67977. A proprietary Genentech EST sequence was employed in the assembly and is herein designated DNA66217.

Based on the DNA67977 consensus sequence, oligonucleotide probes were generated and used to screen a human breast carcinoma (LIB135) library prepared as described in paragraph 1 above. The cloning vector was pRK5B (pRK5B is a precursor of pRK5D that does not contain the SfiI site; see, Holmes et al., Science, 253:1278-1280 (1991)), and the cDNA size cut was less than 2800 bp.

PCR primers (forward and reverse) were synthesized:

| | |
|---|---|
| forward PCR primer (67977.f1) | |
| 5′-TCCTTCGGCTGCTGTGATCAGCAC-3′ | (SEQ ID NO: 229) |
| reverse PCR primer (67977.r1) | |
| 5′-CCCAGGTGGCGGTTGAGATAGTCG-3′ | (SEQ ID NO: 230) |

Additionally, a synthetic oligonucleotide hybridization probe was constructed from the consensus DNA67977 sequence which had the following nucleotide sequence

hybridization probe (67977.p1)
(SEQ ID NO: 231)
5′-CGCTGCCCGGTACTGGGACATCATGGAATATTTTGATCTGAAGAG-3′

In order to screen several libraries for a source of a full-length clone, DNA from the libraries was screened by PCR amplification with the PCR primer pair identified above. A positive library was then used to isolate clones encoding the PRO1779 gene using the probe oligonucleotide and one of the PCR primers.

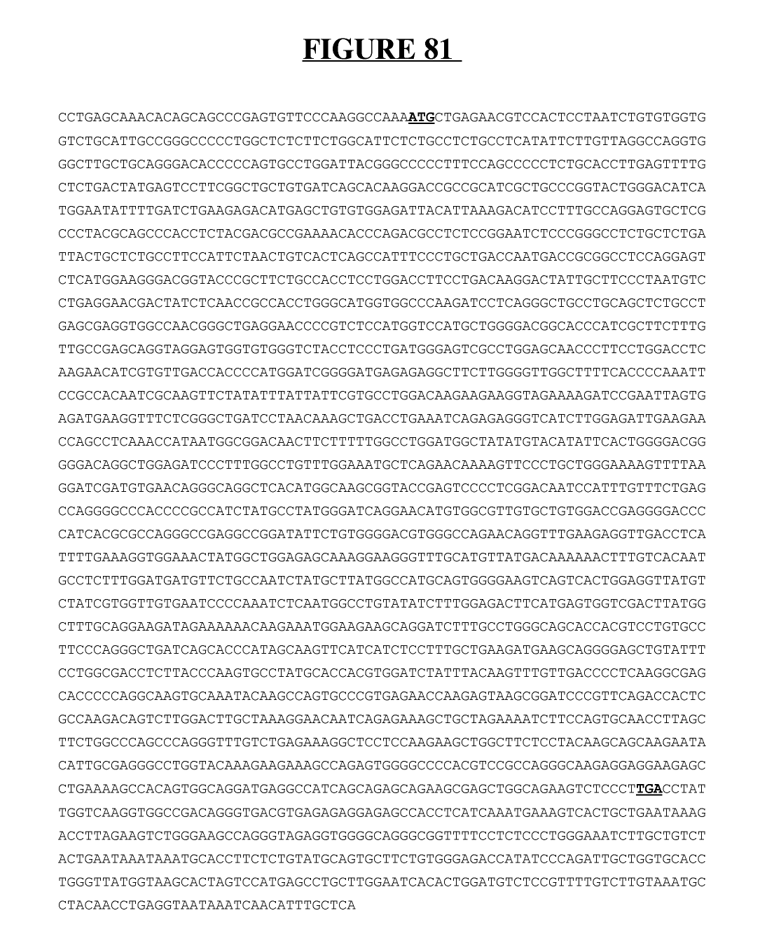

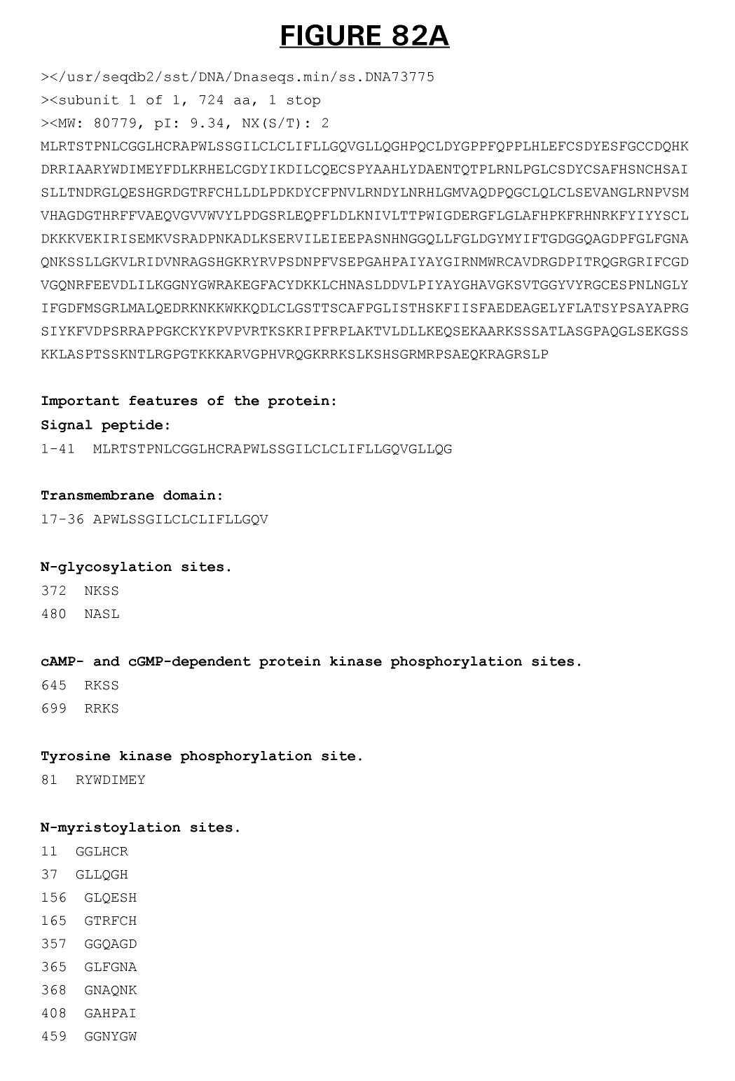

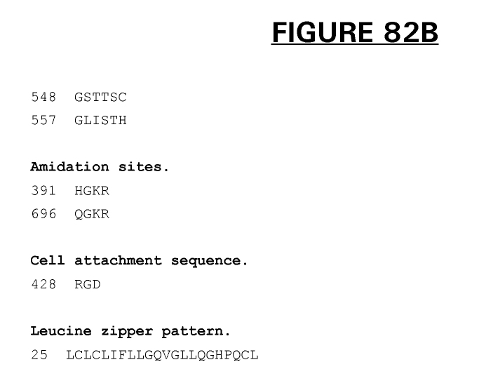

A full length clone was identified that contained a single open reading frame with an apparent translational initiation site at nucleotide positions 41-43, and a stop signal at nucleotide positions 2213-2215 (FIG. 81, SEQ ID NO:81). The predicted polypeptide precursor is 724 amino acids long, has a calculated molecular weight of approximately 80,779 daltons and an estimated pI of approximately 9.34. Analysis of the full-length PRO1779 sequence shown in FIG. 82A-82B (SEQ ID NO:82) evidences the presence of the following: a signal peptide from about amino acid 1 to about amino acid 41, a transmembrane domain from about amino acid 17 to about amino acid 36, potential N-glycosylation sites from about amino acid 372 to about amino acid 375 and from about amino acid 480 to about amino acid 483, cAMP- and cGMP-dependent protein kinase phosphorylation sites from about amino acid 645 to about amino acid 648 and from about amino acid 699 to about amino acid 702, a tyrosine kinase phosphorylation site from about amino acid 81 to about amino acid 88, potential N-myristolation sites from about amino acid 11 to about amino acid 16, from about amino acid 37 to about amino acid 42, from about amino acid 156 to about amino acid 161, from about amino acid 165 to about amino acid 170, from about amino acid 357 to about amino acid 362, from about amino acid 365 to about amino acid 370, from about amino acid 368 to about amino acid 373, from about amino acid 408 to about amino acid 413, from about amino acid 459 to about amino acid 464, from about amino acid 548 to about amino acid 553 and from about amino acid 557 to about amino acid 562, amidation sites from about amino acid 391 to about amino acid 394 and from about amino acid 696 to about amino acid 699, a cell attachment sequence from about amino acid 428 to about amino acid 430 and a leucine zipper pattern sequence from about amino acid 25 to about amino acid 46. Clone UNQ841 (DNA73775-1707) has been deposited with ATCC on May 25, 1999 and is assigned ATCC deposit no. PTA-128.

An analysis of the Dayhoff database (version 35.45 SwissProt 35), using a WU-BLAST2 sequence alignment analysis of the full-length sequence shown in FIG. 82A-82B (SEQ ID NO:82), evidenced significant homology between the PRO1779 amino acid sequence and the following Dayhoff sequences: XLU37373_{—}1, P_W56538, S74981, E64821, P_W56540, AF083072_{—}2, VTA2_XENLA, Y112_HUMAN, STE2_YEAST and SON_HUMAN.

### Example 43

### Isolation of cDNA Clones Encoding Human PRO1785 Polypeptides [UNQ847]

A consensus DNA sequence was assembled relative to other EST sequences using phrap as described in Example 1 above. This consensus sequence is designated herein “DNA35718”. Based on the DNA35718 consensus sequence, oligonucleotides were synthesized: 1) to identify by PCR a cDNA library that contained the sequence of interest, and 2) for use as probes to isolate a clone of the full-length coding sequence for PRO1785.

PCR primers (forward and reverse) were synthesized:

| | |
|---|---|
| forward PCR primer: | |
| 5′-ATCCTCCAACATGGAGCCTCTTGC-3′; | (SEQ ID NO: 232) |
| forward PCR primer: | |
| 5′-GTATCTTGTCAACCCTGAGG-3′; | (SEQ ID NO: 233) |
| and | |
| reverse PCR primer: | |
| 5′-TAACCAGAGCTGCTATGTCAGGCC-3′; | (SEQ ID NO: 234) |

Additionally, a synthetic oligonucleotide hybridization probe was constructed from the consensus DNA35718 sequence which had the following nucleotide sequence:

hybridization probe:
(SEQ ID NO: 235)
5′-AGGCAAAGTTTCACTAGTTGTAAACGTGGCCAGTGACTGCCAACTC
ACAG-3′.

In order to screen several libraries for a source of a full-length clone, DNA from the libraries was screened by PCR amplification with the PCR primer pair identified above. A positive library was then used to isolate clones encoding the PRO1785 gene using the probe oligonucleotide and one of the PCR primers. RNA for construction of the cDNA libraries was isolated from human aortic endothelial cells.

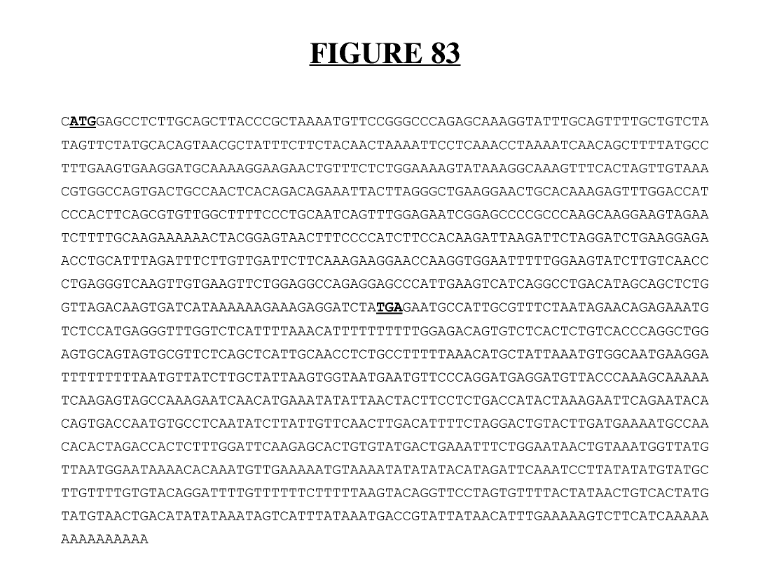

DNA sequencing of the clones isolated as described above gave the full-length DNA sequence for PRO1785 (designated herein as DNA80136-2503 [FIG. 83, SEQ ID NO:83]; and the derived protein sequence for PRO1785.

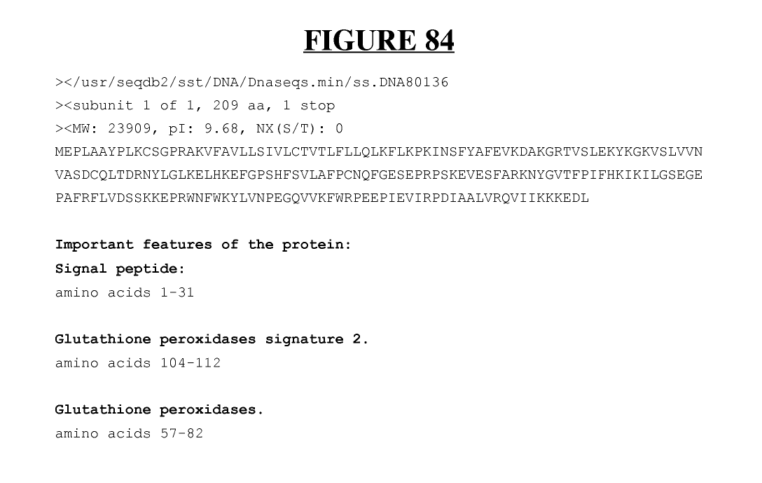

The entire coding sequence of PRO1785 is shown in FIG. 83 (SEQ ID NO:83). Clone DNA80136-2503 contains a single open reading frame with an apparent translational initiation site at nucleotide positions 2-4 and an apparent stop codon at nucleotide positions 629-631 of SEQ ID NO:83. The predicted polypeptide precursor is 209 amino acids long. There is a signal peptide at about amino acids 1-31, a transmembrane domain at about amino acids 18-37 and a glutathione peroxidase signature at about amino acids 104-111 of SEQ ID NO:84. Clone DNA80136-2503 has been deposited with the ATCC (Dec. 15, 1998) and is assigned ATCC deposit no. 203541. The full-length PRO1785 protein shown in FIG. 84 has an estimated molecular weight of about 23,909 daltons and a pI of about 9.68.

An analysis of the Dayhoff database (version 35.45 SwissProt 35), using a WU-BLAST2 sequence alignment analysis of the full-length sequence shown in FIG. 84 (SEQ ID NO:84), revealed sequence identity between the PRO1785 amino acid sequence and the following Dayhoff sequences: GSHC_SCHMA, P_R44988, AB012395_{—}1, GSHH_HUMAN, AC004151_{—}3, BTUE ECOLI, GSHC HUMAN, P_R89910, PWU88907_{—}1 and D37916_{—}1.

### Example 44

### Isolation of cDNA Clones Encoding Human PRO1889 Polypeptides [UNQ_{871]}

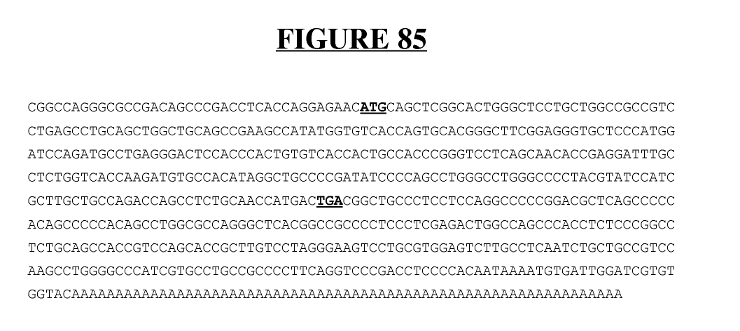

A consensus DNA sequence was assembled relative to other EST sequences using phrap as described in Example 1 above. This consensus sequence is herein designated DNA49310. Based up an observed homology between the DNA49310 consensus sequence and an EST contained within the Incyte EST clone no. 2779436, Incyte EST clone no. 2779436 was purchased and its insert obtained and sequenced. The sequence of that insert is shown in FIG. 85 and is herein designated DNA77623-2524.

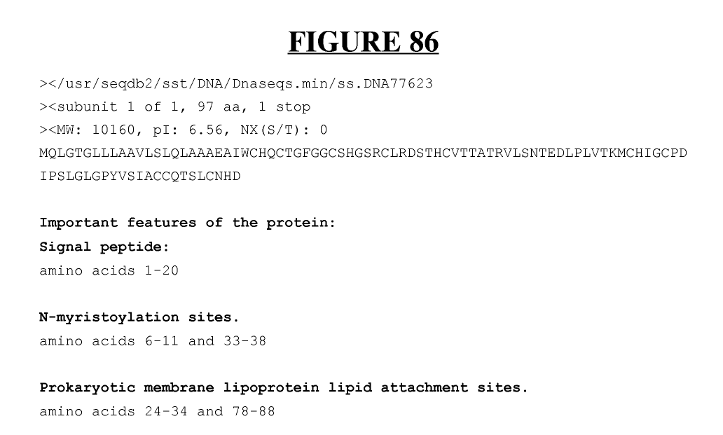

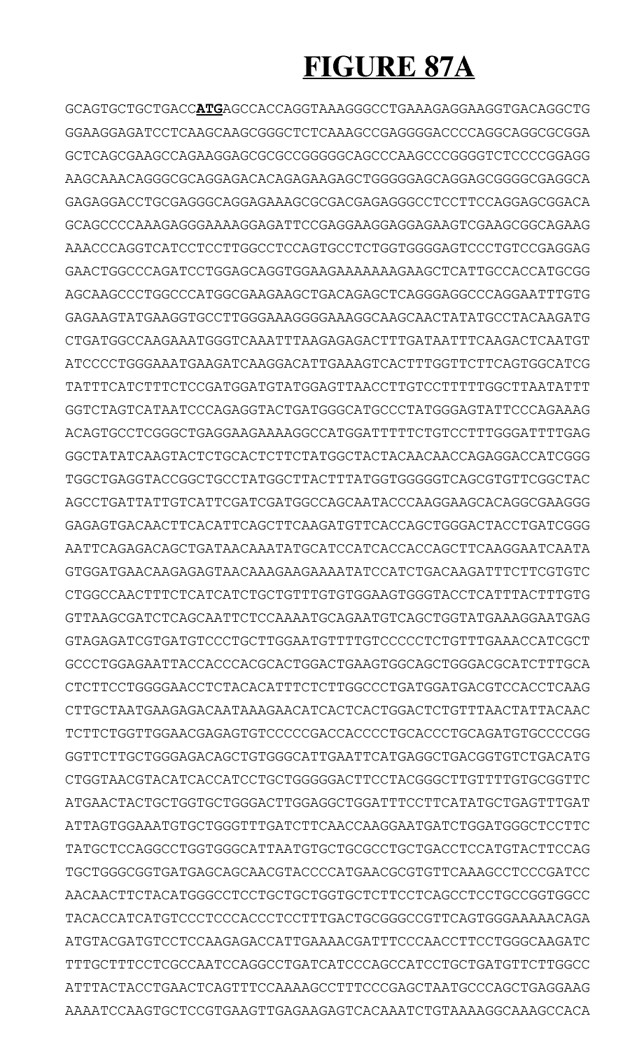

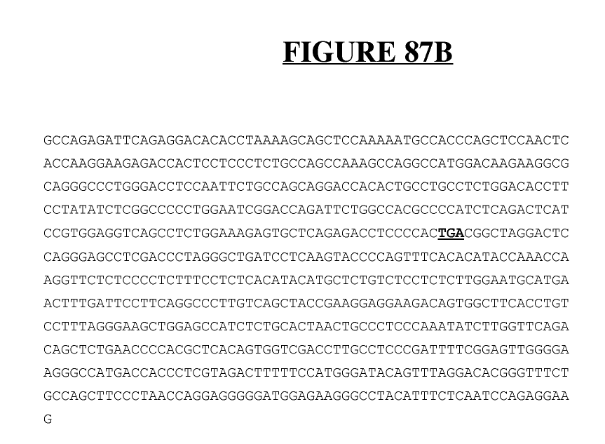

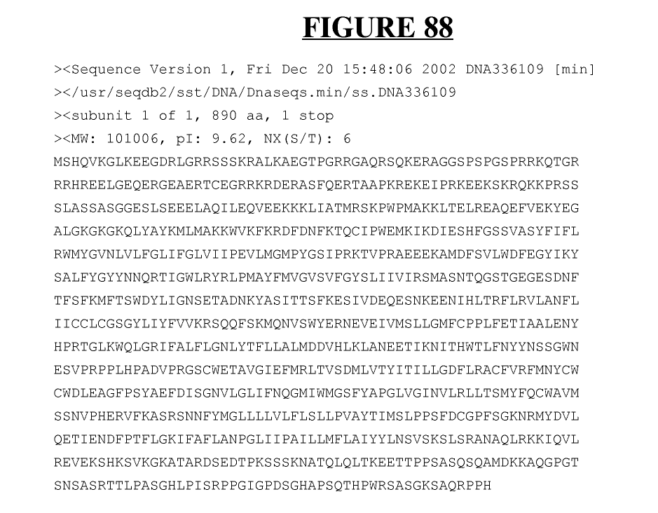

The entire nucleotide sequence of DNA77623-2524 is shown in FIG. 85 (SEQ ID NO:85). Clone DNA77623-2524 contains a single open reading frame with an apparent translational initiation site at nucleotide positions 39-41 and ending at the stop codon at nucleotide positions 330-332 (FIG. 85). The predicted polypeptide precursor is 97 amino acids long (FIG. 86). The full-length PRO1889 protein shown in FIG. 86 has an estimated molecular weight of about 10,160 daltons and a pI of about 6.56. Analysis of the full-length PRO1889 sequence shown in FIG. 86 (SEQ ID NO:86) evidences the presence of the following: a signal peptide from about amino acid 1 to about amino acid 20, potential N-myristolation sites from about amino acid 6 to about amino acid 11 and from about amino acid 33 to about amino acid 38 and prokaryotic membrane lipoprotein lipid attachment sites from about amino acid 24 to about amino acid 34 and from about amino acid 78 to about amino acid 88. Clone DNA77623-2524 has been deposited with ATCC on Dec. 22, 1998 and is assigned ATCC deposit no. 203546.

An analysis of the Dayhoff database (version 35.45 SwissProt 35), using a WU-BLAST2 sequence alignment analysis of the full-length sequence shown in FIG. 86 (SEQ ID NO:86), evidenced significant homology between the PRO1889 amino acid sequence and the following Dayhoff sequences: HSE48ATGN_{—}1, P_W06292, AB012293_{—}1, THYB_MOUSE, P_R70984, CHKSCA2A_{—}1, P_W61628, I48639, BMBUNGKP4_{—}1 and UPAR_HUMAN.

### Example 45

### Isolation of cDNA Clones Encoding Human PRO3434 Polypeptides [UNQ1821]

DNA77631-2537 was identified by applying the proprietary signal sequence finding algorithm described in Example 2 above. Use of the above described signal sequence algorithm allowed identification of an ESTcluster sequence from the LIFESEQ® database. This EST cluster sequence was then compared to a variety of expressed sequence tag (EST) databases which included public EST databases (e.g., GenBank) and a proprietary EST DNA database (LIFESEQ®, Incyte Pharmaceuticals, Palo Alto, Calif.) to identify existing homologies. The homology search was performed using the computer program BLAST or BLAST2 (Altshul et al., Methods in Enzymology, 266:460-480 (1996)). Those comparisons resulting in a BLAST score of 70 (or in some cases 90) or greater that did not encode known proteins were clustered and assembled into a consensus DNA sequence with the program “phrap” (Phil Green, University of Washington, Seattle, Wash.). The consensus sequence obtained therefrom is herein designated as DNA56099.

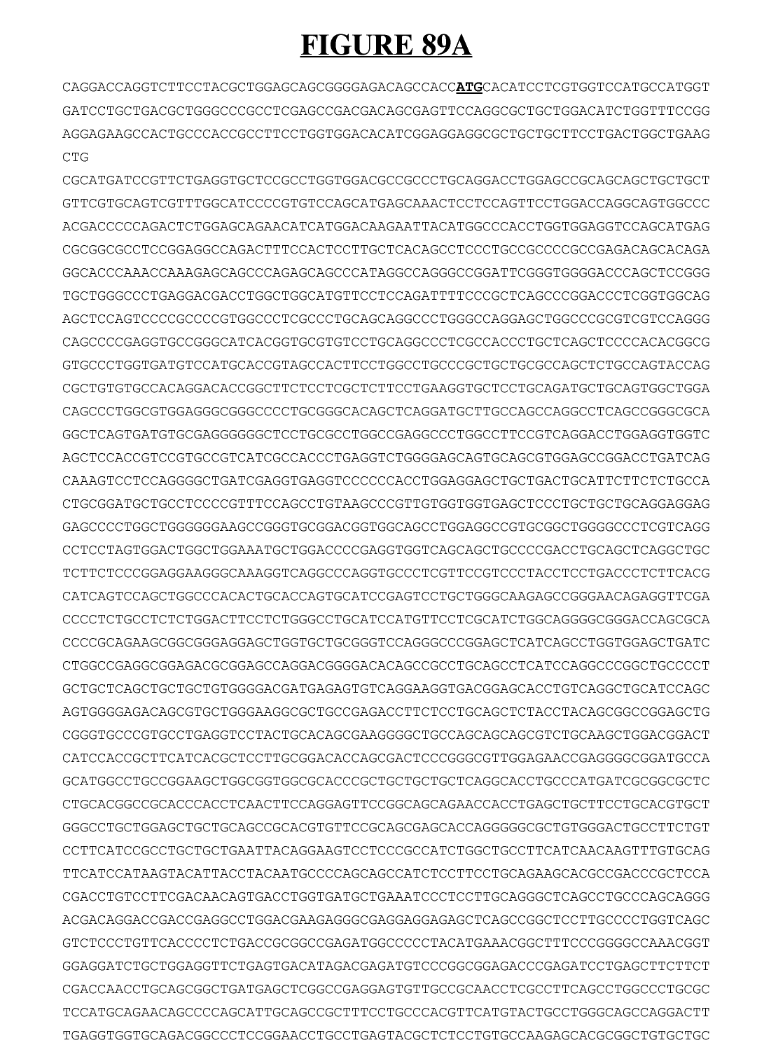

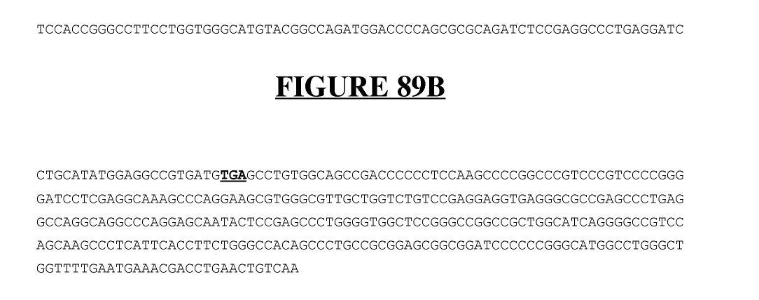

In light of the sequence homology between the DNA56099 sequence and Incyte EST clone no. 3327089, Incyte EST clone no. 3327089 was purchased and the cDNA insert was obtained and sequenced. The sequence of this cDNA insert is shown in FIG. 89A-89B (SEQ ID NO:89) and is herein designated as DNA77631-2537.

Clone DNA77631-2537 contains a single open reading frame with an apparent translational initiation site at nucleotide positions 46-48 and ending at the stop codon at nucleotide positions 3133-3135 (FIG. 89A-89B).

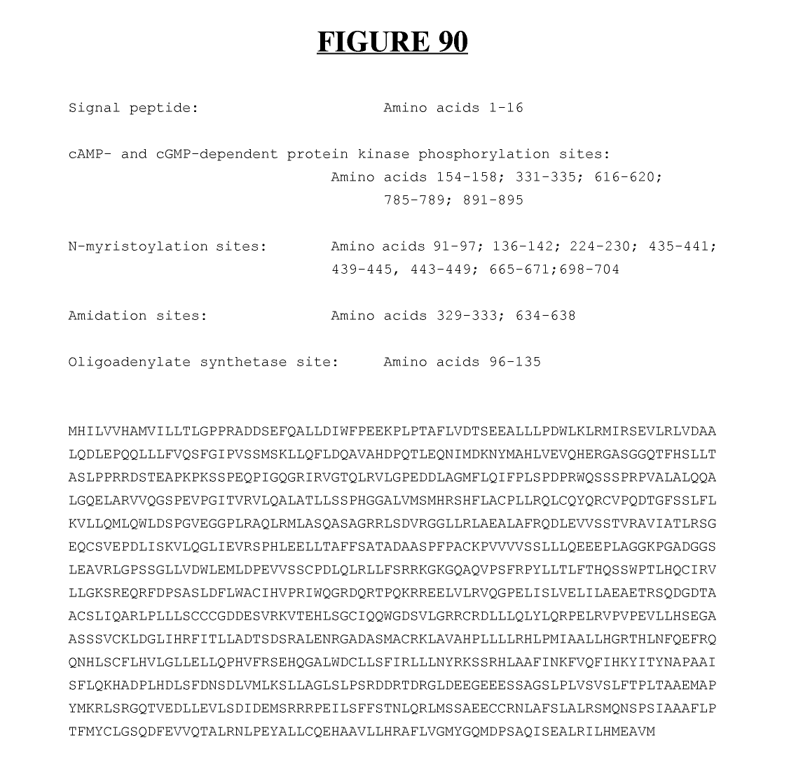

The predicted polypeptide precursor is 1029 amino acids long (FIG. 90; SEQ ID NO:90). The full-length PRO3434 protein shown in FIG. 90 has an estimated molecular weight of about 114,213 daltons and a pI of about 6.42. Analysis of the full-length PRO3434 sequence shown in FIG. 90 (SEQ ID NO:90) evidences the presence of a variety of important polypeptide domains, wherein the locations given for those important polypeptide domains are approximate as described above. Analysis of the full-length PRO3434 sequence shown in FIG. 90 evidences the presence of the following: a signal peptide from about amino acid 1 to about amino acid 16; cAMP- and cGMP-dependent protein kinase phosphorylation sites from about amino acid 154 to about amino acid 158, from about amino acid 331 to about amino acid 335, from about amino acid 616 to about amino acid 620, from about amino acid 785 to about amino acid 789, and from about amino acid 891 to about amino acid 895; potential N-myristoylation sites from about amino acid 91 to about amino acid 97, from about amino acid 136 to about amino acid 142, from about amino acid 224 to about amino acid 230, from about amino acid 435 to about amino acid 441, from about amino acid 439 to about amino acid 445, from about amino acid 443 to about amino acid 449, from about amino acid 665 to about amino acid 671, and from about amino acid 698 to about amino acid 704; amidation sites from about amino acid 329 to about amino acid 333, and from about amino acid 634 to about amino acid 638; and an oligoadenylate synthetase site from about amino acid 96 to about amino acid 135. Clone DNA77631-2537 has been deposited with ATCC on Feb. 9, 1999 and is assigned ATCC deposit no. 203651.

An analysis of the Dayhoff database (version 35.45 SwissProt 35), using a WU-BLAST2 sequence alignment analysis of the full-length sequence shown in FIG. 90 (SEQ ID NO:90), revealed significant sequence identity between the PRO3434 amino acid sequence and the following Dayhoff sequences: VATX_YEAST, P_R51171, POLS_IBDVP, IBDVORF_{—}2, JC5043, IBDVPIV_{—}1, VE7—HPV11, GEN14220, MUT_THETH and COAC_CHICK.

### Example 46

### Isolation of cDNA Clones Encoding Human PRO3579 Polypeptides [UNQ1849]

DNA68862-2546 was identified by applying a proprietary signal sequence finding algorithm developed by Genentech, Inc. (South San Francisco, Calif.) upon ESTs as well as clustered and assembled EST fragments from public (e.g., GenBank) and/or private (LIFESEQ®, Incyte Pharmaceuticals, Inc., Palo Alto, Calif.) databases. The signal sequence algorithm computes a secretion signal score based on the character of the DNA nucleotides surrounding the first and optionally the second methionine codon(s) (ATG) at the 5′-end of the sequence or sequence fragment under consideration. The nucleotides following the first ATG must code for at least 35 unambiguous amino acids without any stop codons. If the first ATG has the required amino acids, the second is not examined. If neither meets the requirement, the candidate sequence is not scored. In order to determine whether the EST sequence contains an authentic signal sequence, the DNA and corresponding amino acid sequences surrounding the ATG codon are scored using a set of seven sensors (evaluation parameters) known to be associated with secretion signals.

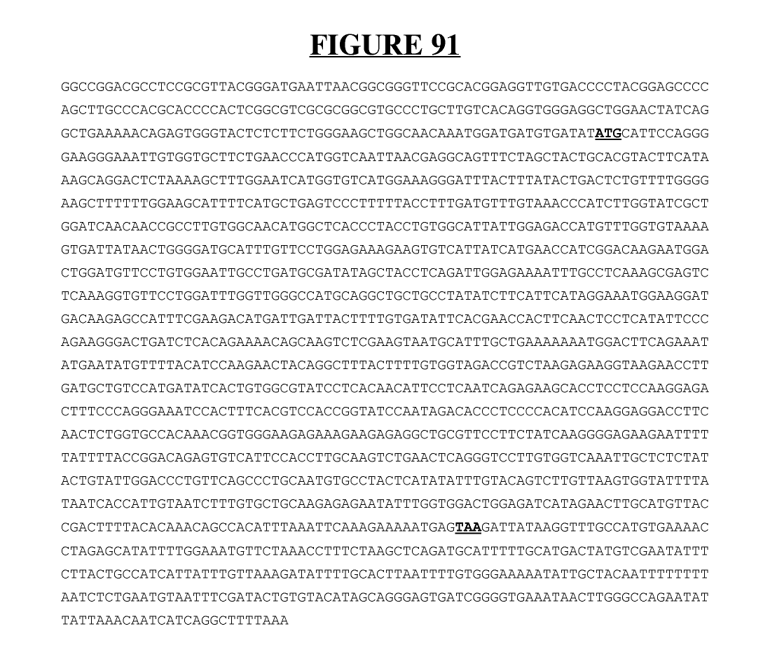

Use of the above described signal sequence algorithm allowed identification of an EST sequence from the Incyte database, designated Incyte EST clone no. 3040247. This EST sequence was then compared to a variety of expressed sequence tag (EST) databases which included public EST databases (e.g., GenBank) and a proprietary EST DNA database (Lifeseq®, Incyte Pharmaceuticals, Palo Alto, Calif.) to identify existing homologies. The homology search was performed using the computer program BLAST or BLAST2 (Altshul et al., Methods in Enzymology 266:460-480 (1996)). Those comparisons resulting in a BLAST score of 70 (or in some cases 90) or greater that did not encode known proteins were clustered and assembled into a consensus DNA sequence with the program “phrap” (Phil Green, University of Washington, Seattle, Wash.). The consensus sequence is herein designated DNA57723. In light of the sequence homology between the DNA57723 sequence and the Incyte EST clone no. 2377329, the Incyte EST clone no. 2377329 was purchased and the cDNA insert was obtained and sequenced. The sequence of this cDNA insert is shown in FIG. 91 and is herein designated as DNA68862-2546.

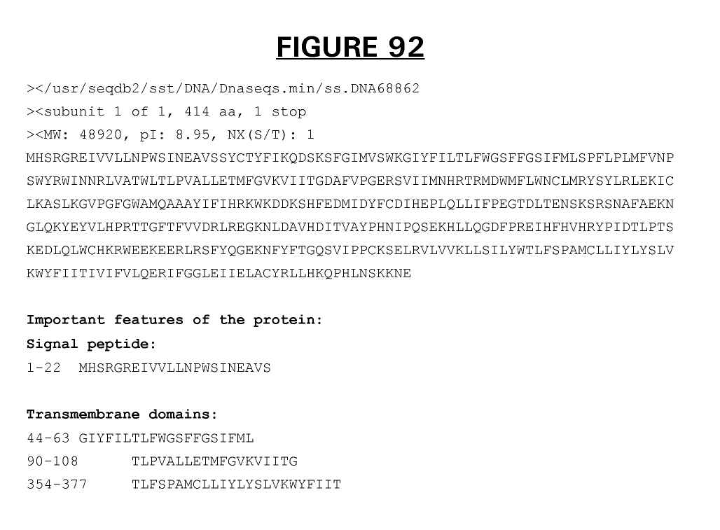

Clone UNQ1849 (DNA68862-2546) contains a single open reading frame with an apparent translational initiation site at nucleotide positions 210-212 and ending at the stop codon at nucleotide positions 1452-1454 (FIG. 91; SEQ ID NO:91). The predicted polypeptide precursor is 414 amino acids long (FIG. 92; SEQ ID NO:92). The full-length PRO3579 protein shown in FIG. 92 has an estimated molecular weight of about 48,920 daltons and a pI of about 8.95. Analysis of the full-length PRO3579 sequence shown in FIG. 92 (SEQ ID NO:92) evidences the presence of a variety of important polypeptide domains as shown in FIG. 92. Clone UNQ1849 (DNA68862-2546) has been deposited with ATCC on Feb. 9, 1999 and is assigned ATCC deposit no. 203652.

An analysis of the Dayhoff database (version 35.45 SwissProt 35), using a WU-BLAST2 sequence alignment analysis of the full-length sequence shown in FIG. 92 (SEQ ID NO:92), evidenced significant homology between the PRO3579 amino acid sequence and the following Dayhoff sequences: CELT05H4_{—}15, CELZK40_{—}1, A38840_{—}1, S52645, P_R99249, YBP2_YEAST, P_R59713, BNAGPATRF_{—}1, D86960_{—}1 and YIHG_ECOLI.

### Example 47

### Isolation of cDNA Clones Encoding Human PRO4322 Polypeptides [UNQ1879]

A cDNA clone designated herein as (DNA92223-2567) encoding a native human PRO4322 polypeptide was identified by a yeast screen, in a human tissue cDNA library that preferentially represents the 5′ ends of the primary cDNA clones.

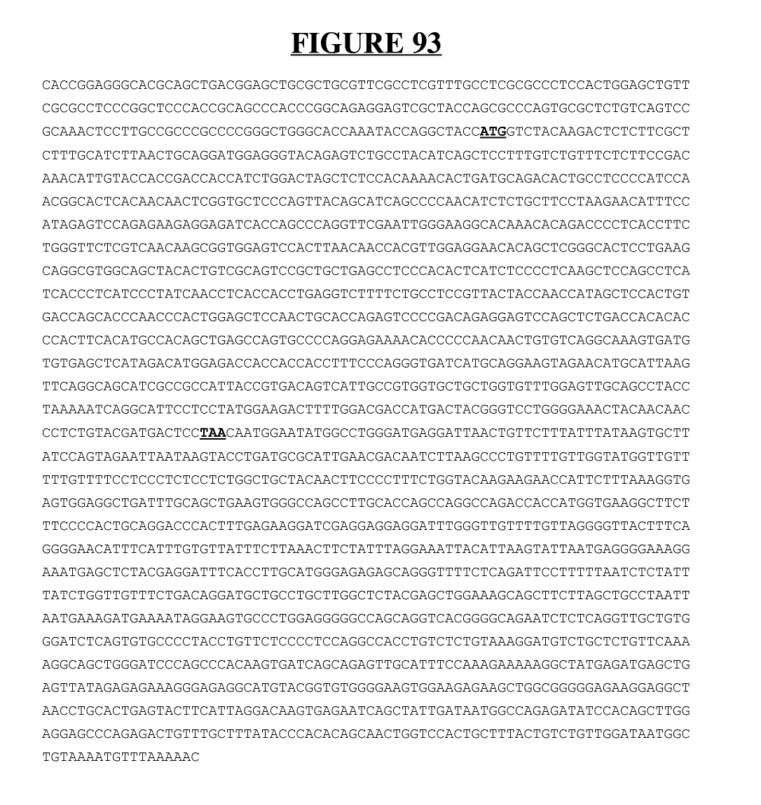

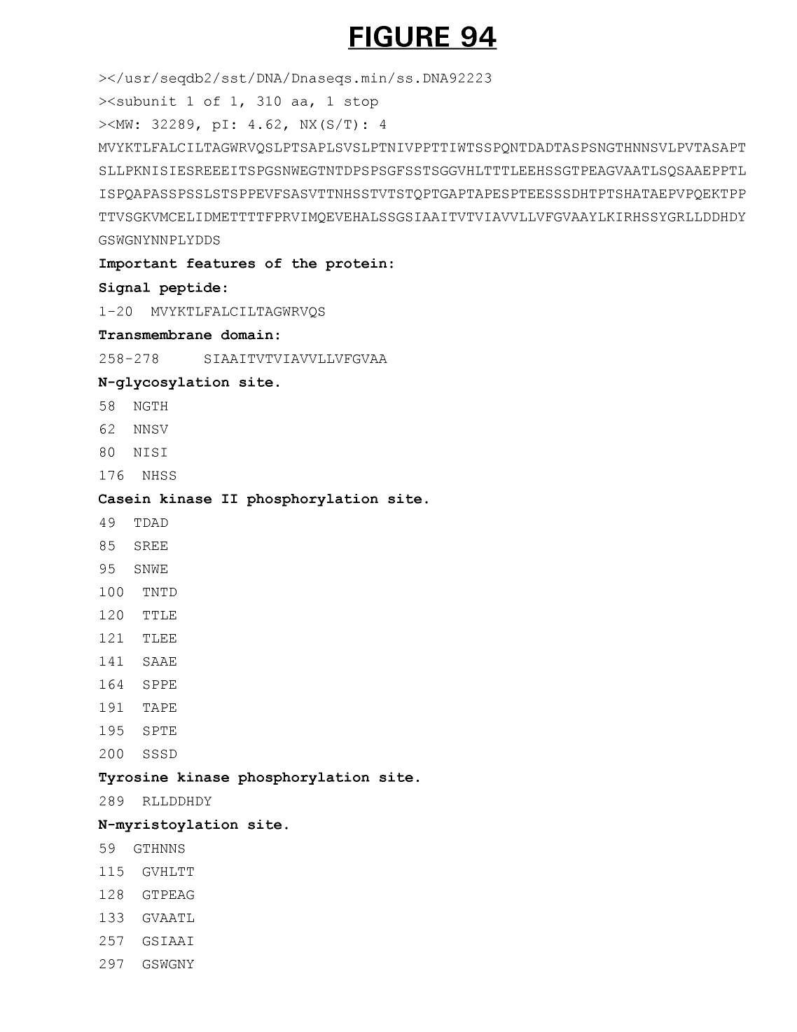

The full-length DNA92223-2567 clone shown in FIG. 93 contains a single open reading frame with an apparent translational initiation site at nucleotide positions 199-201 and ending at the stop codon at nucleotide positions 1129-1131 (FIG. 93; SEQ ID NO:93). The predicted polypeptide precursor is 310 amino acids long (FIG. 94; SEQ ID NO:94). The full-length PRO4322 protein shown in FIG. 94 has an estimated molecular weight of about 32,289 daltons and a pI of about 4.62. Analysis of the full-length PRO4322 sequence shown in FIG. 94 (SEQ ID NO:94) evidences the presence of a variety of important polypeptide domains as shown in FIG. 94. Clone UNQ1879 (DNA92223-2567) has been deposited with ATCC on Mar. 16, 1999 and is assigned ATCC deposit no. 203851.

An analysis of the Dayhoff database (version 35.45 SwissProt 35), using a WU-BLAST2 sequence alignment analysis of the full-length sequence shown in FIG. 94 (SEQ ID NO:94), evidenced some degree of homology between the PRO4322 amino acid sequence and the following Dayhoff sequences: AMYH_YEAAST, MUC2_HUMAN, RNMUCASGP7_{—}1, C114_MOUSE, AGA1_YEAST, D88734_{—}1, VGP3_EBVA8, P_P91941, A37232 and FIG2_—YEAST.

### Example 48

### Isolation of cDNA Clones Encoding Human PRO4343 Polypeptides [UNQ1897]

DNA92255-2584 was identified by applying a proprietary signal sequence finding algorithm developed by Genentech, Inc. (South San Francisco, Calif.) upon ESTs as well as clustered and assembled EST fragments from public (e.g., GenBank) and/or private (LIFESEQ®, Incyte Pharmaceuticals, Inc., Palo Alto, Calif.) databases. The signal sequence algorithm computes a secretion signal score based on the character of the DNA nucleotides surrounding the first and optionally the second methionine codon(s) (ATG) at the 5′-end of the sequence or sequence fragment under consideration. The nucleotides following the first ATG must code for at least 35 unambiguous amino acids without any stop codons. If the first ATG has the required amino acids, the second is not examined. If neither meets the requirement, the candidate sequence is not scored. In order to determine whether the EST sequence contains an authentic signal sequence, the DNA and corresponding amino acid sequences surrounding the ATG codon are scored using a set of seven sensors (evaluation parameters) known to be associated with secretion signals.

Use of the above described signal sequence algorithm allowed identification of an EST cluster sequence from the Incyte database. This EST cluster sequence was then compared to a variety of expressed sequence tag (EST) databases which included public EST databases (e.g., GenBank) and a proprietary EST DNA database (LIFESEQ®, Incyte Pharmaceuticals, Palo Alto, Calif.) to identify existing homologies. The homology search was performed using the computer program BLAST or BLAST2 (Altshul et al., Methods in Enzymology 266:460-480 (1996)). Those comparisons resulting in a BLAST score of 70 (or in some cases 90) or greater that did not encode known proteins were clustered and assembled into a consensus DNA sequence with the program “phrap” (Phil Green, University of Washington, Seattle, Wash.). The consensus sequence is herein designated DNA59225. In light of the sequence DNA59225 sequence DNA92255-2584 was identified.

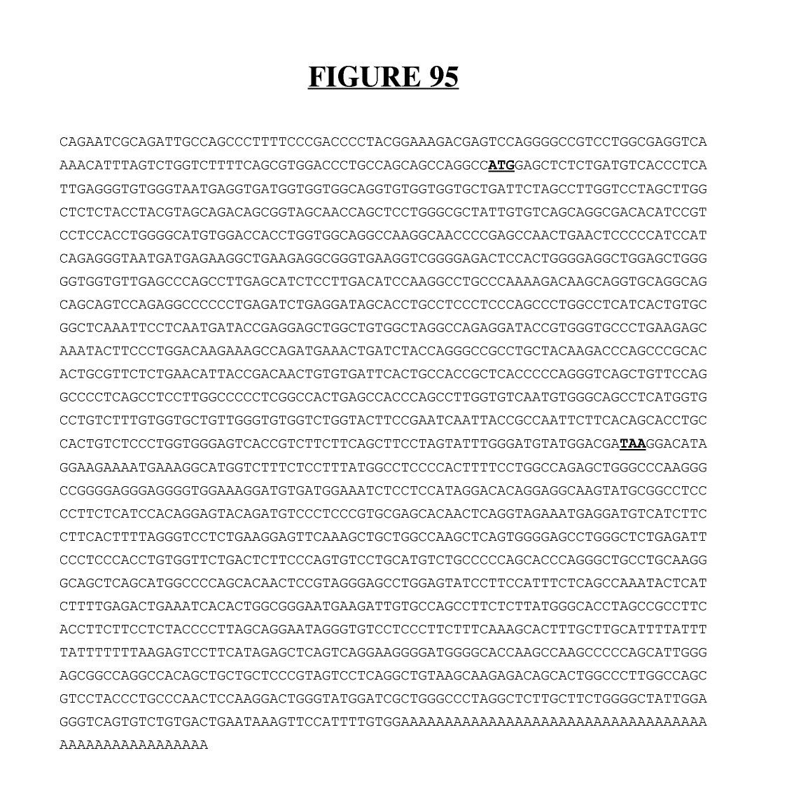

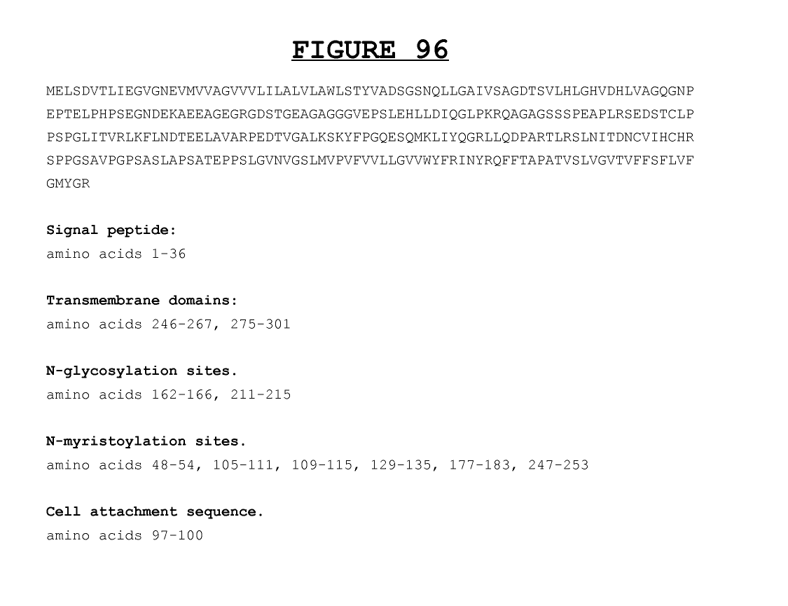

The full length clone shown in FIG. 95 contained a single open reading frame with an apparent translational initiation site at nucleotide positions 124-226 and ending at the stop codon found at nucleotide positions 1027-1029 (FIG. 95; SEQ ID NO:95). The predicted polypeptide precursor (FIG. 96, SEQ ID NO:96) is 301 amino acids long. PRO4343 has a calculated molecular weight of approximately 31607 daltons and an estimated pI of approximately 4.89.

An analysis of the Dayhoff database (version 35.45 SwissProt 35), using a WU-BLAST2 sequence alignment analysis of the full-length sequence shown in FIG. 96 (SEQ ID NO:96), revealed homology between the PRO4343 amino acid sequence and the following Dayhoff sequences (sequences and related text incorporated herein): YKA4_CAEEL, GGARBP_{—}1, TPM5_DROME, DROTRO11_{—}1, P_R60126, CHU45963_{—}1, MMHC188A7_{—}5, AF085809_{—}1, P_R37683, and AF098511_{—}1.

Clone DNA92255-2584 (UNQ1897), designated as DNA92255-2584 was deposited with the ATCC on Mar. 23, 1999 and is assigned ATCC deposit no. 203866.

### Example 49

### Isolation of cDNA Clones Encoding Human PRO4347 Polypeptides [UNQ1901]

The extracellular domain (ECD) sequences (including the secretion signal sequence, if any) from about 950 known secreted proteins from the Swiss-Prot public database were used to search EST databases. The EST databases included public EST databases (e.g., GenBank), and a proprietary EST database (LIFESEQ®, Incyte Pharmaceuticals, Palo Alto, Calif.). The search was performed using the computer program BLAST or BLAST2 [Altschul et al., Methods in Enzymology, 266:460-480 (1996)] as a comparison of the ECD protein sequences to a 6 frame translation of the EST sequences. Those comparisons resulting in a BLAST score of 70 (or in some cases, 90) or greater that did not encode known proteins were clustered and assembled into consensus DNA sequences with the program “phrap” (Phil Green, University of Washington, Seattle, Wash.).

A consensus DNA sequence encoding PRO4347 was assembled relative to other EST sequences using phrap and extended using repeated cycles. This consensus sequence is designated herein “DNA77498”.

Based on the DNA77498 consensus sequence a clone was identified and sequenced.

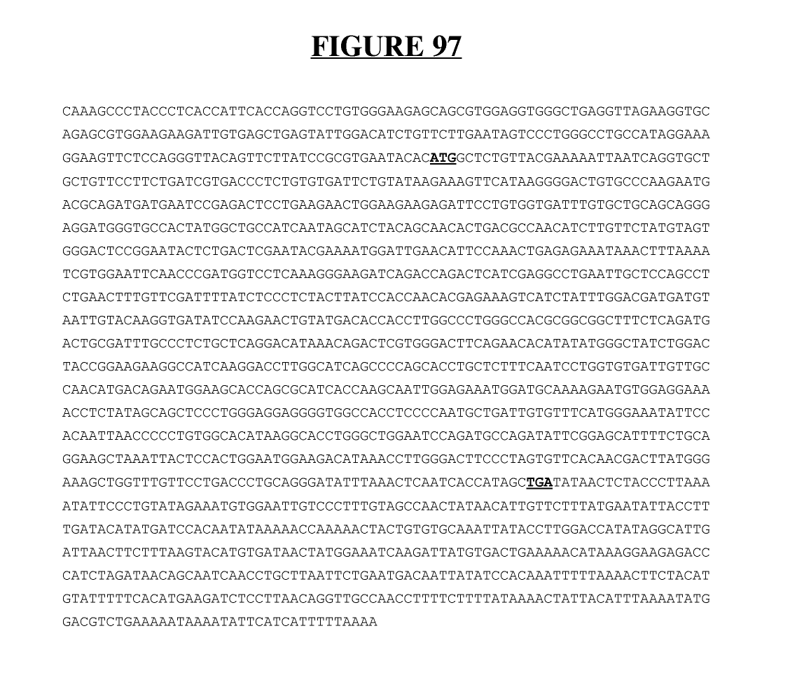

DNA sequencing of the clones isolated as described above gave the full-length DNA sequence for PRO4347 (designated herein as DNA92288-2588 [FIG. 97, SEQ ID NO:97]; and the derived protein sequence for PRO4347.

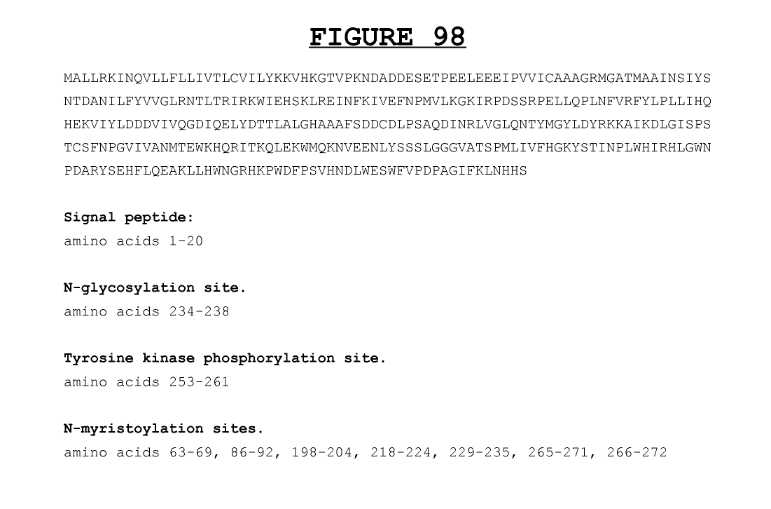

The entire coding sequence of PRO4347 is shown in FIG. 97 (SEQ ID NO:97). Clone DNA92288-2588 contains a single open reading frame with an apparent translational initiation site at nucleotide positions 191-193, and an apparent stop codon at nucleotide positions 1238-1240. The predicted polypeptide precursor is 349 amino acids long. Clone DNA92288-2588 (UNQ1901), designated as DNA92288-2588 has been deposited with ATCC (Mar. 30, 1999) and is assigned ATCC deposit no. 203892 The full-length PRO4347 protein shown in FIG. 98 has an estimated molecular weight of about 40026 daltons and a pI of about 7.0.

An analysis of the Dayhoff database (version 35.45 SwissProt 35), using a WU-BLAST2 sequence alignment analysis of the full-length sequence shown in FIG. 98 (SEQ ID NO:98), revealed homology between the PRO4347 amino acid sequence and the following Dayhoff sequences: F20P5_{—}18, ATAC00549915, Y258_HAEIN, C64146, NMU65788_{—}3, AF019745_{—}6, AB020211_{—}2, GSPA_BACSU, P_R91313 and RFALSALTY.

### Example 50

### Isolation of cDNA Clones Encoding Human PRO4403 Polypeptides [UNQ1928]

The extracellular domain (ECD) sequences (including the secretion signal sequence, if any) from about 950 known secreted proteins from the Swiss-Prot public database were used to search EST databases. The EST databases included public EST databases (e.g., GenBank), and a proprietary EST database (LIFESEQ®, Incyte Pharmaceuticals, Palo Alto, Calif.). The search was performed using the computer program BLAST or BLAST2 [Altschul et al., Methods in Enzymology, 266:460-480 (1996)] as a comparison of the ECD protein sequences to a 6 frame translation of the EST sequences. Those comparisons resulting in a BLAST score of 70 (or in some cases, 90) or greater that did not encode known proteins were clustered and assembled into consensus DNA sequences with the program “phrap” (Phil Green, University of Washington, Seattle, Wash.).

A consensus DNA sequence encoding PRO4403 was assembled relative to other EST sequences using repeated cycles of phrap.

Based on the consensus sequence oligonucleotides were synthesized: 1) to identify by PCR a cDNA library that contained the sequence of interest, and 2) for use as probes to isolate a clone of the full-length coding sequence for PRO4403. Forward and reverse PCR primers generally range from 20 to 30 nucleotides and are often designed to give a PCR product of about 100-1000 by in length. The probe sequences are typically 40-55 by in length. In some cases, additional oligonucleotides are synthesized when the consensus sequence is greater than about 1-1.5 kbp. In order to screen several libraries for a full-length clone, DNA from the libraries was screened by PCR amplification, as per Ausubel et al., Current Protocols in Molecular Biology, supra, with the PCR primer pair. A positive library was then used to isolate clones encoding the gene of interest using the probe oligonucleotide and one of the primer pairs.

PCR primers (forward and reverse) were synthesized:

| | |
|---|---|
| forward PCR primer | |
| 5′GCTTTCATTGCCACGTGGAGTATG3′ | (SEQ ID NO: 236) |
| and | |
| reverse PCR primer | |
| 5′ACCTAGTGAGGCTGGGATTTGGC3′. | (SEQ ID NO: 237) |

Additionally, a synthetic oligonucleotide hybridization probe was constructed from the consensus sequence which had the following nucleotide sequence:

hybridization probe
(SEQ ID NO: 238)
5′CCGCCTGGCTCTGTGCCAAGCCCTTCAAAGTCATCTGTAT3′.

In order to screen several libraries for a source of a full-length clone, DNA from the libraries was screened by PCR amplification with the PCR primer pair identified above. A positive library was then used to isolate clones encoding the PRO4403 gene using the probe oligonucleotide and one of the PCR primers.

RNA for construction of the cDNA libraries was isolated from a human adenocarcinoma cell line. The cDNA libraries used to isolate the cDNA clones were constructed by standard methods using commercially available reagents such as those from Invitrogen, San Diego, Calif. The cDNA was primed with oligo dT containing a NotI site, linked with blunt to SalI hemikinased adaptors, cleaved with NotI, sized appropriately by gel electrophoresis, and cloned in a defined orientation into a suitable cloning vector (such as pRKB or pRKD; pRK5B is a precursor of pRK5D that does not contain the SfiI site; see, Holmes et al., Science, 253:1278-1280 (1991)) in the unique XhoI and NotI sites.

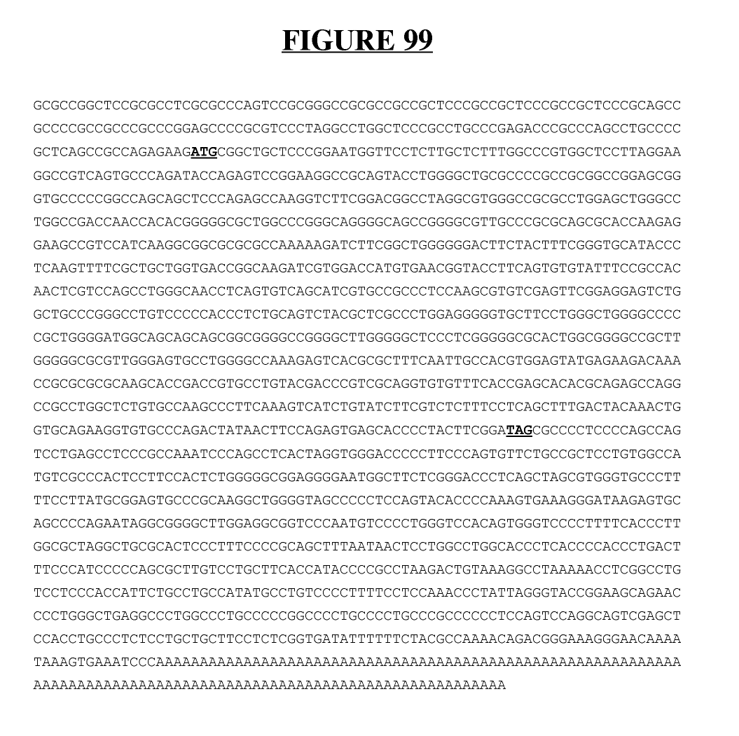

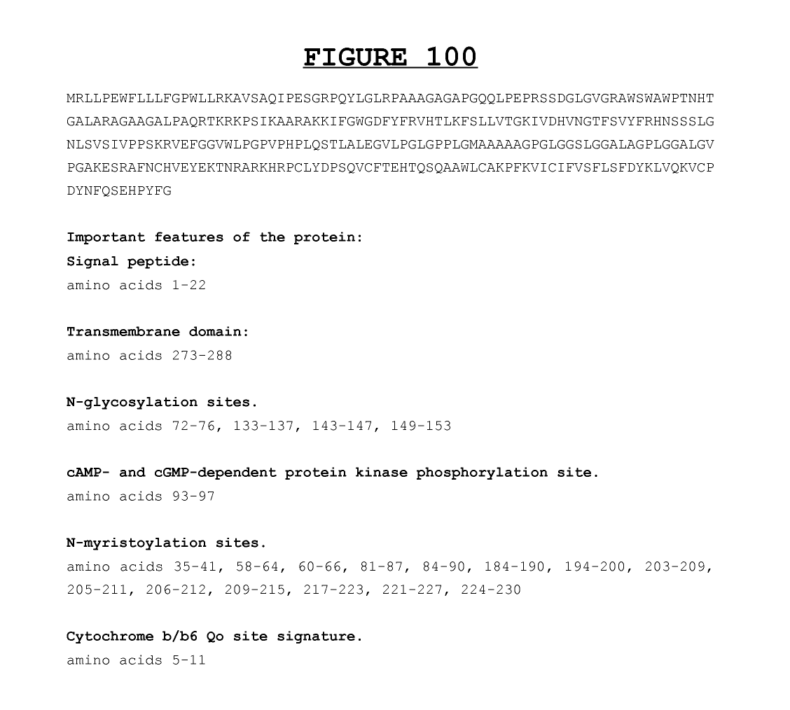

DNA sequencing of the clones isolated as described above gave the full-length DNA sequence for PRO4403 (designated herein as DNA83509-2612 [FIG. 99, SEQ ID NO:99]; and the derived protein sequence for PRO4403.

The entire coding sequence of PRO4403 is shown in FIG. 99 (SEQ ID NO:99). Clone DNA83509-2612 contains a single open reading frame with an apparent translational initiation site at nucleotide positions 167-169, and an apparent stop codon at nucleotide positions 1090-1093. The predicted polypeptide precursor is 308 amino acids long. Clone DNA83509-2612 (UNQ1928), designated as DNA83509-2612 has been deposited with ATCC (Apr. 27, 1999) and is assigned ATCC deposit no. 203965. The full-length PRO4403 protein shown in FIG. 100 has an estimated molecular weight of about 33065 daltons and a pI of about 10.13.

An analysis of the Dayhoff database (version 35.45 SwissProt 35), using a WU-BLAST2 sequence alignment analysis of the full-length sequence shown in FIG. 100 (SEQ ID NO:100), revealed homology between the PRO4403 amino acid sequence and the following Dayhoff sequences (sequences and related text incorporated herein): AF042714_{—}1, AC004613_{—}1, NPH1_RAT, NPH2_BOVIN, AF043467_{—}1, AF043469_{—}1, AF043468_{—}1, ELS_MOUSE, AF029249_{—}1, and K2C3_BOVIN.

### Example 51

### Isolation of cDNA Clones Encoding Human PRO4976 Polypeptides [UNQ2419]

An initial DNA sequence (DNA90650) was identified using a yeast screen, that preferentially represents the 5′ ends of the primary cDNA clones. This sequence was compared to ESTs from public databases (e.g., GenBank), and a proprietary EST database (LIFESEQ®, Incyte Pharmaceuticals, Palo Alto, Calif.), using the computer program BLAST or BLAST2 [Altschul et al., Methods in Enzymology, 266:460-480 (1996)]. The ESTs were clustered and assembled into a consensus DNA sequence using the computer program “phrap” (Phil Green, University of Washington, Seattle, Wash.). This consensus sequence is designated herein as “DNA94848”. Based on the DNA94848 consensus sequence, the following oligonucleotides were synthesized for use as probes to isolate a clone of the full-length coding sequence for PRO1377 from a human human fetal kidney cDNA library: 5′CCGCCAGAAGAATGCAGTTCTG3′ (forward, SEQ ID NO:239), 5′CCTCCACCCTCAGAACTGCCTC 3′(reverse, SEQ ID NO: 240), and 5′GCAATTGGAGCAGTGGAGAAAGACGTGGGCCTGTCGGATG3′ (plasmid, SEQ ID NO:241).

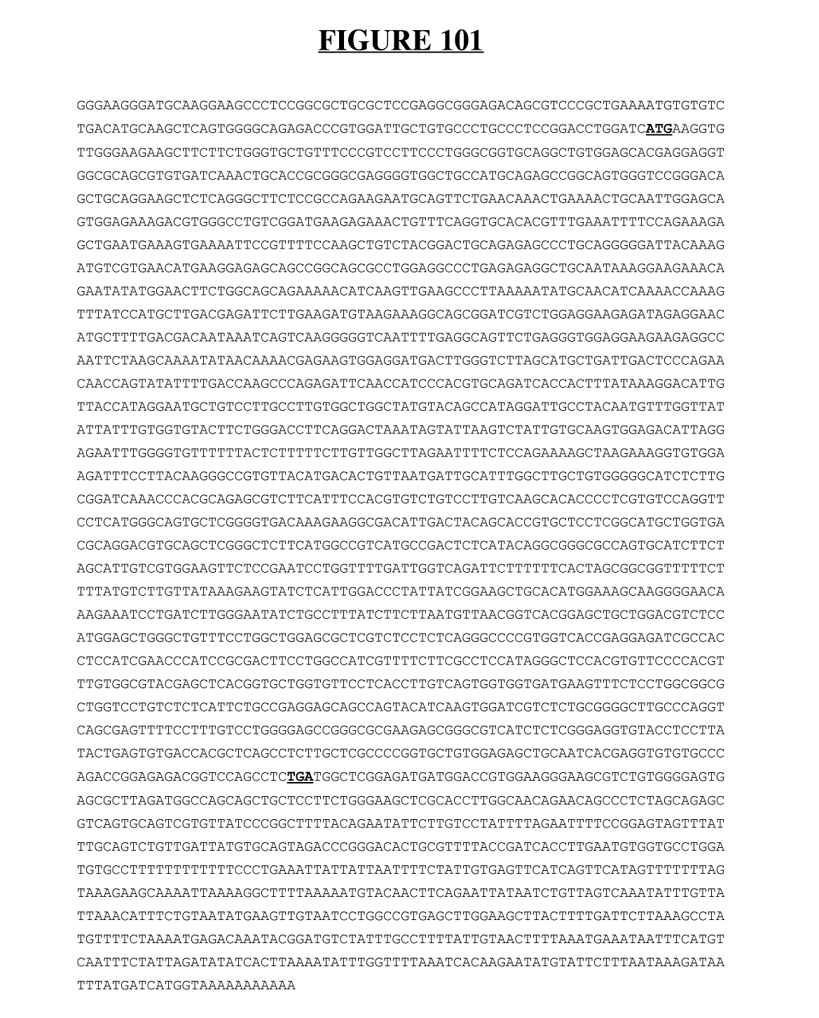

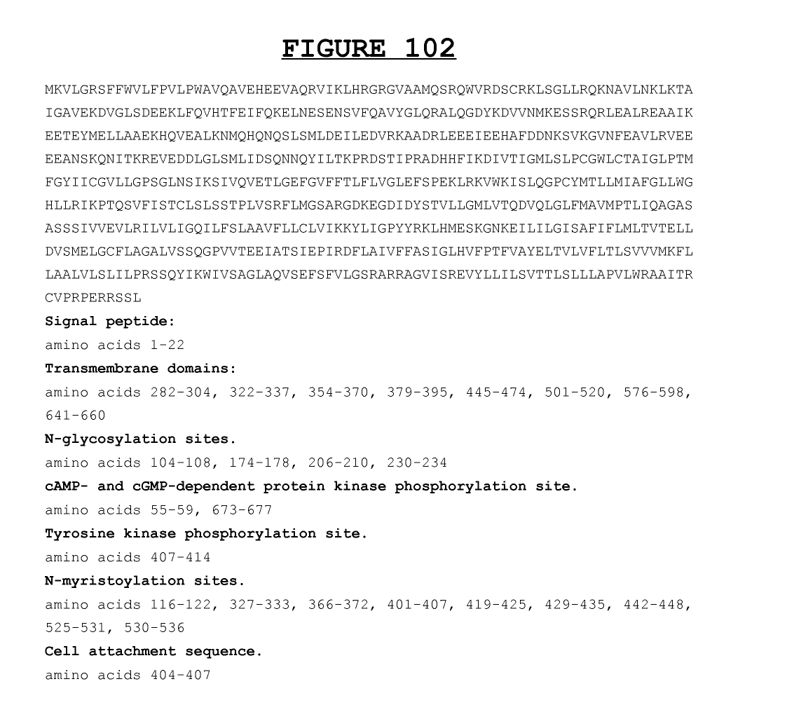

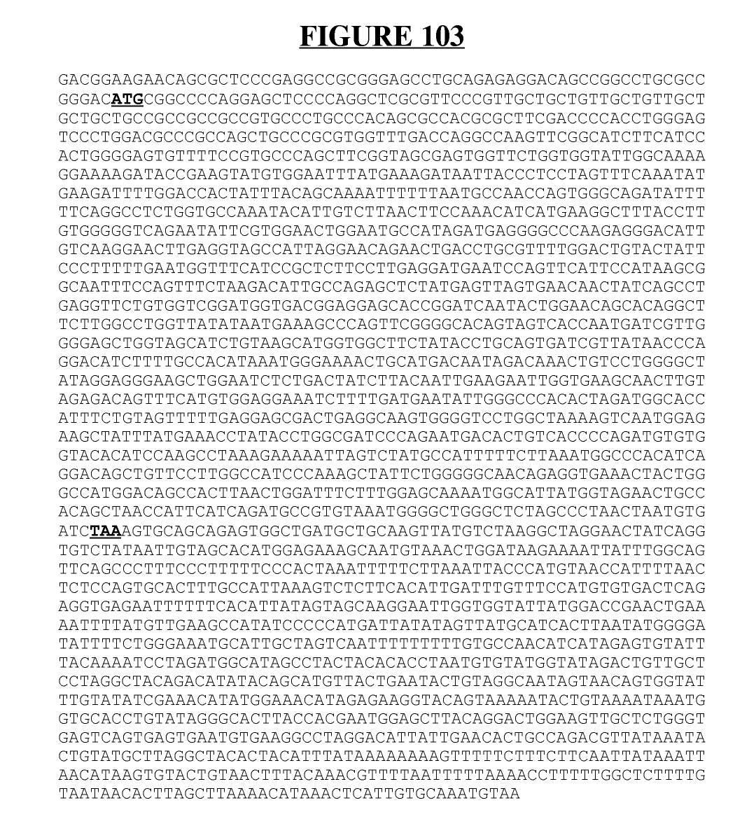

The full length DNA100902-2646 clone shown in FIG. 101 contained a single open reading frame with an apparent translational initiation site at nucleotide positions 140-142 and ending at the stop codon found at nucleotide positions 2171-2173 (FIG. 101; SEQ ID NO:101). The predicted polypeptide precursor (FIG. 102, SEQ ID NO:102) is 677 amino acids long. PRO4976 has a calculated molecular weight of approximately 75598 daltons and an estimated pI of approximately 6.85.

An analysis of the Dayhoff database (version 35.45 SwissProt 35), using a WU-BLAST2 sequence alignment analysis of the full-length sequence shown in FIG. 102 (SEQ ID NO:102), revealed homology between the PRO4976 amino acid sequence and the following Dayhoff sequences: ATAC00591720, MGNMAGA_{—}1, P_W27454, E64778, KEFB_ECOLI, D71642, G69819, T4B21_{—}16, MXU37008_{—}2, and F70591.

Clone DNA100902-2646 (UNQ2419), designated as DNA100902-2646 has been deposited with ATCC (May 11, 1999) and is assigned ATCC deposit no. PTA-42.

### Example 52

### Isolation of cDNA Clones Encoding Human PRO260 Polypeptides (UNQ227)

A consensus DNA sequence was assembled relative to other EST sequences using phrap as described in Example 1 above. This consensus sequence is herein designated DNA30834. Based on the DNA30834 consensus sequence, oligonucleotides were synthesized: 1) to identify by PCR a cDNA library that contained the sequence of interest, and 2) for use as probes to isolate a clone of the full-length coding sequence for PRO260.

PCR primers (forward and two reverse) were synthesized:

| | |
|---|---|
| forward PCR primer: | |
| 5′-TGGTTTGACCAGGCCAAGTTCGG-3′; | (SEQ ID NO: 242) |
| reverse PCR primer A: | |
| 5′-GGATTCATCCTCAAGGAAGAGCGG-3′; | (SEQ ID NO: 243) |
| and | |
| reverse PCR primer B: | |
| 5′AACTTGCAGCATCAGCCACTCTGC-3′ | (SEQ ID NO: 244) |

Additionally, a synthetic oligonucleotide hybridization probe was constructed from the consensus DNA30834 sequence which had the following nucleotide sequence:

hybridization probe:
(SEQ ID NO: 245)
5′-TTCCGTGCCCAGCTTCGGTAGCGAGTGGTTCTGGTGGTATTGGCA-3′

In order to screen several libraries for a source of a full-length clone, DNA from the libraries was screened by PCR amplification with the PCR primer pair identified above. A positive library was then used to isolate clones encoding the PRO260 gene using the probe oligonucleotide and one of the PCR primers.

RNA for construction of the cDNA libraries was isolated from human fetal kidney tissue.

DNA sequencing of the clones isolated as described above gave the full-length DNA sequence for PRO260 [herein designated as DNA33470-1175] (SEQ ID NO:103) and the derived protein sequence for PRO260.

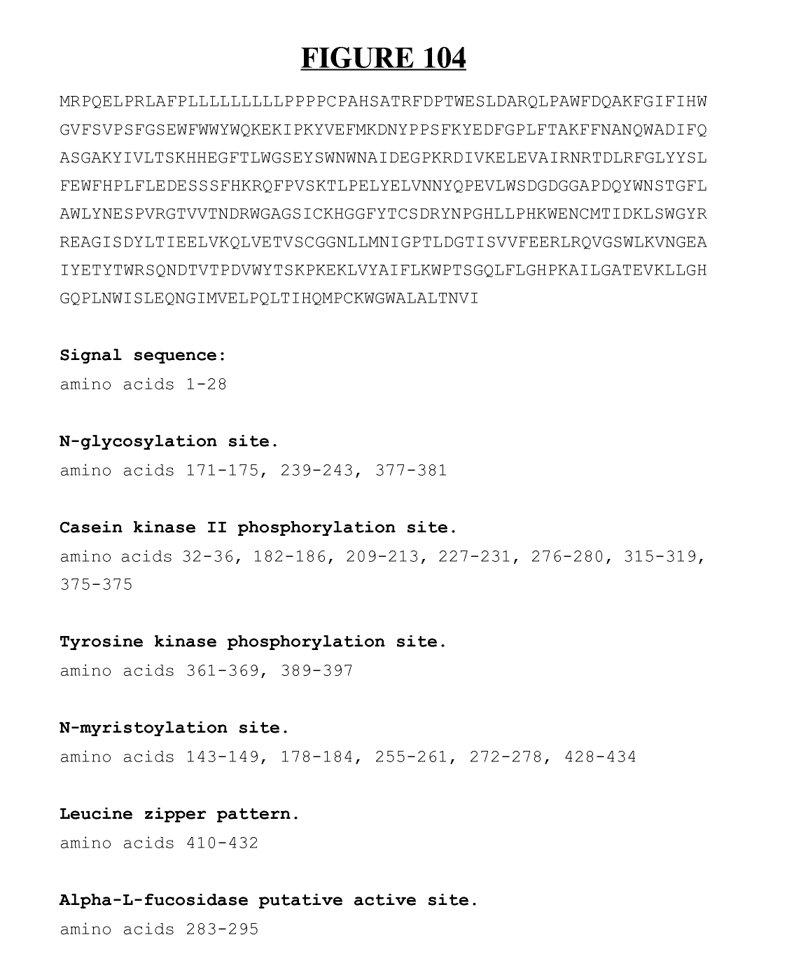

The entire nucleotide sequence of DNA33470-1175 is shown in FIG. 103 (SEQ ID NO:103). Clone DNA33470-1175 contains a single open reading frame with an apparent translational initiation site at nucleotide positions 67-69 and ending at the stop codon 1468-1470 (see FIG. 103). The predicted polypeptide precursor is 467 amino acids long (FIG. 104; SEQ ID NO:104). Clone DNA33470-1175 has been deposited with ATCC on Oct. 17, 1997 and is assigned ATCC deposit no. ATCC 209398.

Analysis of the amino acid sequence of the full-length PRO260 polypeptide (FIG. 104; SEQ ID NO:104) suggests that portions of it possess significant homology to the alpha-1-fucosidase precursor, thereby indicating that PRO260 may be a novel fucosidase.

### Example 53

### Isolation of cDNA Clones Encoding Human PRO6014 Polypeptides [UNQ2521]

DNA92217-2697 was identified by applying a proprietary signal sequence finding algorithm developed by Genentech, Inc. (South San Francisco, Calif.) upon ESTs as well as clustered and assembled EST fragments from public (e.g., Genbank) and/or private (LIFESEQ®, Incyte Pharmaceuticals, Inc., Palo Alto, Calif.) databases. The signal sequence algorithm computes a secretion signal score based on the character of the DNA nucleotides surrounding the first and optionally the second methionine codon(s) (ATG) at the 5′-end of the sequence or sequence fragment under consideration. The nucleotides following the first ATG must code for at least 35 unambiguous amino acids without any stop codons. If the first ATG has the required amino acids, the second is not examined. If neither meets the requirement, the candidate sequence is not scored. In order to determine whether the EST sequence contains an authentic signal sequence, the DNA and corresponding amino acid sequences surrounding the ATG codon are scored using a set of seven sensors (evaluation parameters) known to be associated with secretion signals.

Use of the above described signal sequence algorithm allowed identification of an EST sequence from the LIFESEQ® database, Incyte Pharmaceuticals, Palo Alto, Calif., designated herein as 3402774H1. This EST sequence was then compared to a variety of expressed sequence tag (EST) databases which included public EST databases (e.g., Genbank) and a proprietary EST DNA database (LIFESEQ®, Incyte Pharmaceuticals, Palo Alto, Calif.) to identify existing homologies. The homology search was performed using the computer program BLAST or BLAST2 (Altshul et al., Methods in Enzymology 266:460-480 (1996)). Those comparisons resulting in a BLAST score of 70 (or in some cases 90) or greater that did not encode known proteins were clustered and assembled into a consensus DNA sequence with the program “phrap” (Phil Green, University of Washington, Seattle, Wash.). The consensus sequence obtained therefrom is herein designated DNA79331.

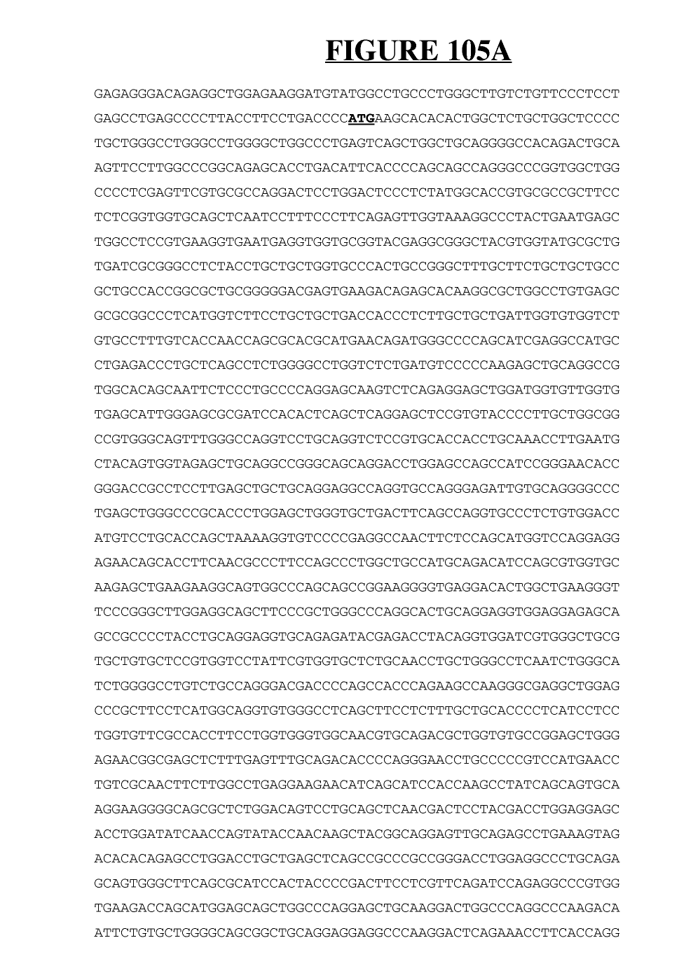

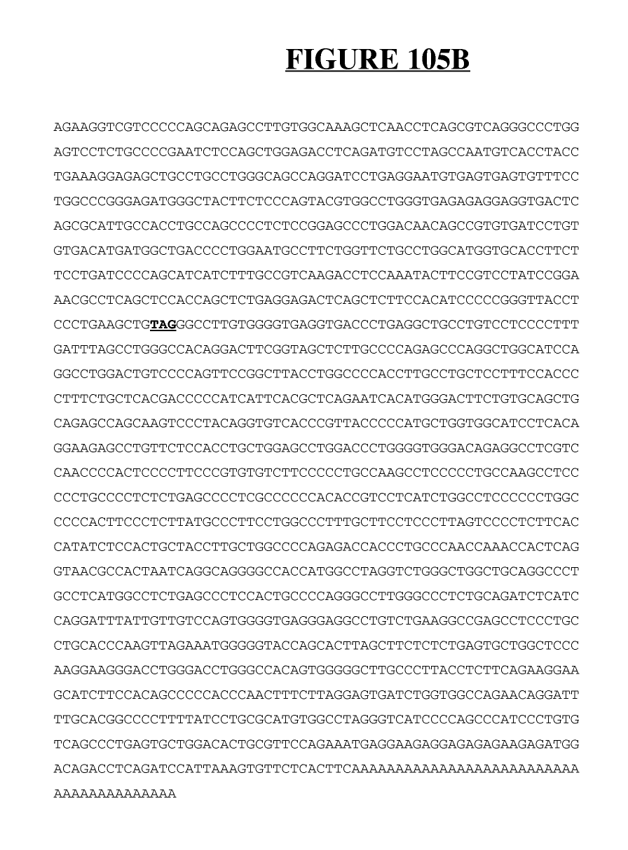

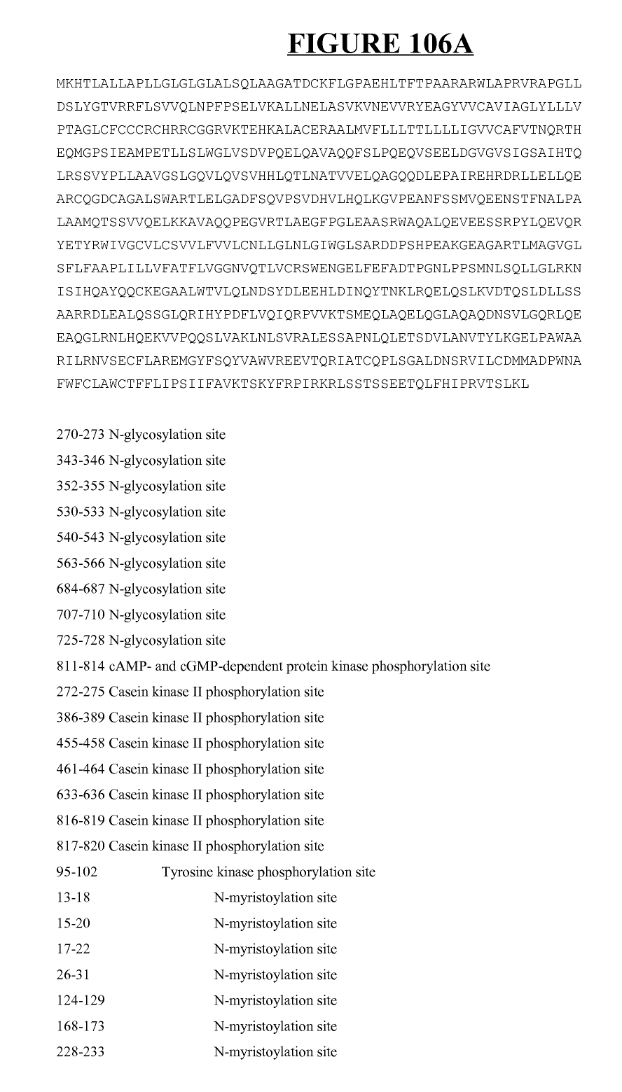

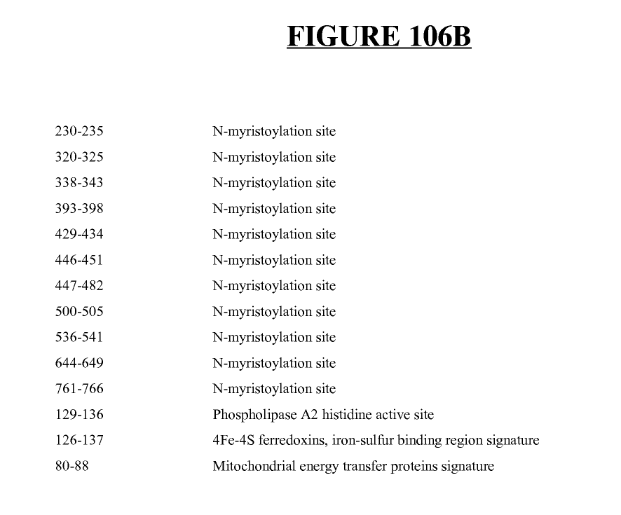

In light of an observed sequence homology between the DNA79331 sequence and an EST sequence encompassed within clone no. 3402774H1 from the LIFESEQ® database, Incyte Pharmaceuticals, Palo Alto, Calif., clone no. 3402774H1 was purchased and the cDNA insert was obtained and sequenced. It was found herein that that cDNA insert encoded a full-length protein. The sequence of this cDNA insert is shown in FIG. 105A-105B (SEQ ID NO:105) and is herein designated as DNA92217-2697.

Clone DNA92217-2697 contains a single open reading frame with an apparent translational initiation site at nucleotide positions 90-92 and ending at the stop codon at nucleotide positions 2592-2594 (FIG. 105A-105B). The predicted polypeptide precursor is 834 amino acids long (FIG. 106A-106B). The full-length PRO6014 protein shown in FIG. 106A-106B has an estimated molecular weight of about 91,911 daltons and a pI of about 6.17. Analysis of the full-length PRO6014 sequence shown in FIG. 106A-106B (SEQ ID NO: 106) evidences the presence of a variety of important polypeptide domains as shown in FIG. 106A-106B, wherein the locations given for those important polypeptide domains are approximate as described above. Clone DNA92217-2697 has been deposited with ATCC on Aug. 10, 1999 and is assigned ATCC Deposit No. PTA-513.

An analysis of the Dayhoff database (version 35.45 SwissProt 35), using the ALIGN-2 sequence alignment analysis of the full-length sequence shown in FIG. 106A-106B (SEQ ID NO: 106), evidenced sequence identity between the PRO6014 amino acid sequence and the following Dayhoff sequences: AF128113_{—}1; AF026269_{—}1; AF039663_{—}1; P_W26769; AF027208_{—}1; AF127935_{—}1; NFL_COTJA; GCMYO2_{—}1; AF014204_{—}1; P_W23996.

### Example 54

### Isolation of cDNA Clones Encoding Human PRO6027 Polypeptides [UNQ2528]

DNA105838-2702 was identified by applying a proprietary signal sequence finding algorithm developed by Genentech, Inc. (South San Francisco, Calif.) upon ESTs as well as clustered and assembled EST fragments from public (e.g., Genbank) and/or private (LIFESEQ®, Incyte Pharmaceuticals, Inc., Palo Alto, Calif.) databases. The signal sequence algorithm computes a secretion signal score based on the character of the DNA nucleotides surrounding the first and optionally the second methionine codon(s) (ATG) at the 5′-end of the sequence or sequence fragment under consideration. The nucleotides following the first ATG must code for at least 35 unambiguous amino acids without any stop codons. If the first ATG has the required amino acids, the second is not examined. If neither meets the requirement, the candidate sequence is not scored. In order to determine whether the EST sequence contains an authentic signal sequence, the DNA and corresponding amino acid sequences surrounding the ATG codon are scored using a set of seven sensors (evaluation parameters) known to be associated with secretion signals.

Use of the above described signal sequence algorithm allowed identification of an EST cluster sequence from the LIFESEQ® (Incyte Pharmaceuticals, Inc., Palo Alto, Calif.) database, designated herein as cluster173032. This EST cluster sequence was then compared to a variety of expressed sequence tag (EST) databases which included public EST databases (e.g., Genbank) and a proprietary EST DNA database (LIFESEQ®, Incyte Pharmaceuticals, Palo Alto, Calif.) to identify existing homologies. The homology search was performed using the computer program BLAST or BLAST2 (Altshul et al., Methods in Enzymology 266:460-480 (1996)). Those comparisons resulting in a BLAST score of 70 (or in some cases 90) or greater that did not encode known proteins were clustered and assembled into a consensus DNA sequence with the program “phrap” (Phil Green, University of Washington, Seattle, Wash.). The consensus sequence obtained therefrom is herein designated DNA59467.

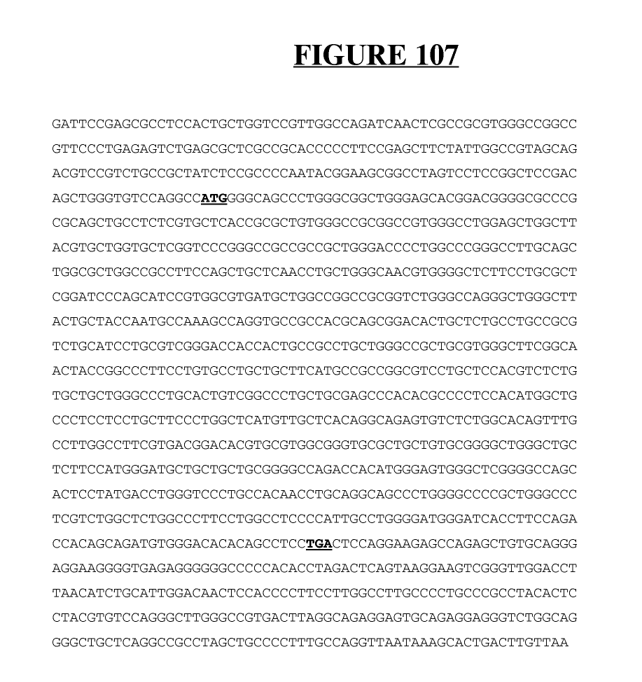

In light of an observed sequence homology between the DNA59467 sequence and an EST sequence encompassed within clone no. 3274259 from the Incyte (Incyte Pharmaceuticals, Inc., Palo Alto, Calif.) database, clone no. 3274259 was purchased and the cDNA insert was obtained and sequenced. It was found herein that that cDNA insert encoded a full-length protein. The sequence of this cDNA insert is shown in FIG. 107 and is herein designated as DNA105838-2702.

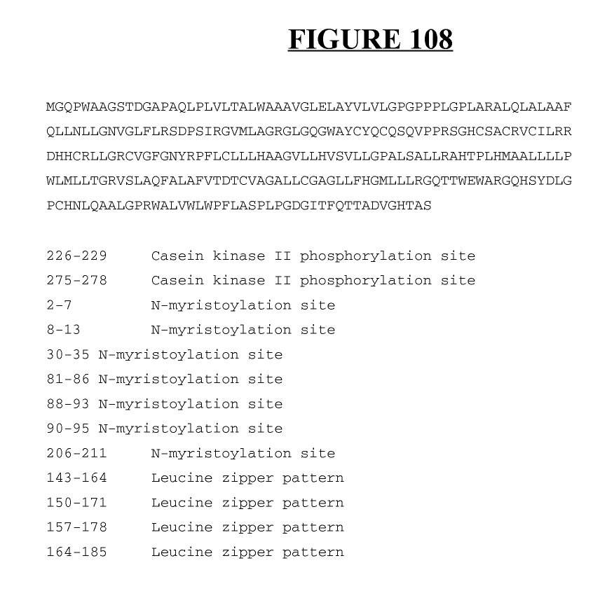

Clone DNA105838-2702 contains a single open reading frame with an apparent translational initiation site at nucleotide positions 198-200 and ending at the stop codon at nucleotide positions 1050-1052 (FIG. 107; SEQ ID NO:107). The predicted polypeptide precursor is 284 amino acids long (FIG. 108; SEQ ID NO:108). The full-length PRO6027 protein shown in FIG. 108 has an estimated molecular weight of about 30176 daltons and a pI of about 9.03. Analysis of the full-length PRO6027 sequence shown in FIG. 108 (SEQ ID NO:108) evidences the presence of a variety of important polypeptide domains as shown in FIG. 108, wherein the locations given for those important polypeptide domains are approximate as described above. Clone DNA105838-2702 has been deposited with ATCC on Aug. 3, 1999 and is assigned ATCC Deposit No. PTA-476.

An analysis of the Dayhoff database (version 35.45 SwissProt 35), using the ALIGN-2 sequence alignment analysis of the full-length sequence shown in FIG. 108 (SEQ ID NO: 108), evidenced sequence identity between the PRO6027 amino acid sequence and the following Dayhoff sequences: CEFO9B12_{—}2; YB3F_SCHPO; S59392; YO41_CAEEL; ATT12H12_{—}14; YA2A_SCHPO; S61981; CAR012683_{—}1; HSU90653_{—}1; S52691.

### Example 55

### Isolation of cDNA Clones Encoding Human PRO6181 Polypeptides [UNQ2552]

The extracellular domain (ECD) sequences (including the secretion signal sequence, if any) from about 950 known secreted proteins from the Swiss-Prot public database were used to search EST databases. The EST databases included public EST databases (e.g., Merck/Washington University). The search was performed using the computer program BLAST or BLAST2 [Altschul et al., Methods in Enzymology, 266:460-480 (1996)] as a comparison of the ECD protein sequences to a 6 frame translation of the EST sequences. Those comparisons resulting in a BLAST score of 70 (or in some cases, 90) or greater that did not encode known proteins were clustered and assembled into consensus DNA sequences with the program “phrap” (Phil Green, University of Washington, Seattle, Wash.).

A consensus DNA sequence was assembled relative to other EST sequences using phrap as described above. This consensus sequence is herein designated DNA80179. In some cases, the DNA80179 consensus sequence derives from an intermediate consensus DNA sequence which was extended using repeated cycles of BLAST and phrap to extend that intermediate consensus sequence as far as possible using the sources of EST sequences discussed above.

Based on the DNA80179 consensus sequence oligonucleotides were synthesized: 1) to identify by PCR a cDNA library that contained the sequence of interest, and 2) for use as probes to isolate a clone of the full-length coding sequence for PRO6181. Forward and reverse PCR primers generally range from 20 to 30 nucleotides and are often designed to give a PCR product of about 100-1000 by in length. The probe sequences are typically 40-55 by in length. In some cases, additional oligonucleotides are synthesized when the consensus sequence is greater than about 1-1.5 kbp. In order to screen several libraries for a full-length clone, DNA from the libraries was screened by PCR amplification, as per Ausubel et al., Current Protocols in Molecular Biology, supra, with the PCR primer pair. A positive library was then used to isolate clones encoding the gene of interest using the probe oligonucleotide and one of the primer pairs.

PCR primers (forward and reverse) were synthesized:

| | |
|---|---|
| forward PCR primer | |
| 5′-CCAGCATTTGAGACTTGTGCAGC-3′ | (SEQ ID NO: 246) |
| reverse PCR primer | |
| 5′-GACTGTAGGAGGCAATGGACACTCC-3′ | (SEQ ID NO: 247) |

Additionally, a synthetic oligonucleotide hybridization probe was constructed from the consensus DNA80179 sequence which had the following nucleotide sequence

hybridization probe
(SEQ ID NO: 248)
5′-CCATCTCCACTCTGCCCGGGCTGGAGCTCTTTTGTGCTATG-3′

RNA for construction of the cDNA libraries was isolated from human testis tissue. The cDNA libraries used to isolate the cDNA clones were constructed by standard methods using commercially available reagents such as those from Invitrogen, San Diego, Calif. The cDNA was primed with oligo dT containing a NotI site, linked with blunt to San hemikinased adaptors, cleaved with NotI, sized appropriately by gel electrophoresis, and cloned in a defined orientation into a suitable cloning vector (such as pRKB or pRKD; pRK5B is a precursor of pRK5D that does not contain the SfiI site; see, Holmes et al., Science, 253:1278-1280 (1991)) in the unique XhoI and NotI sites.

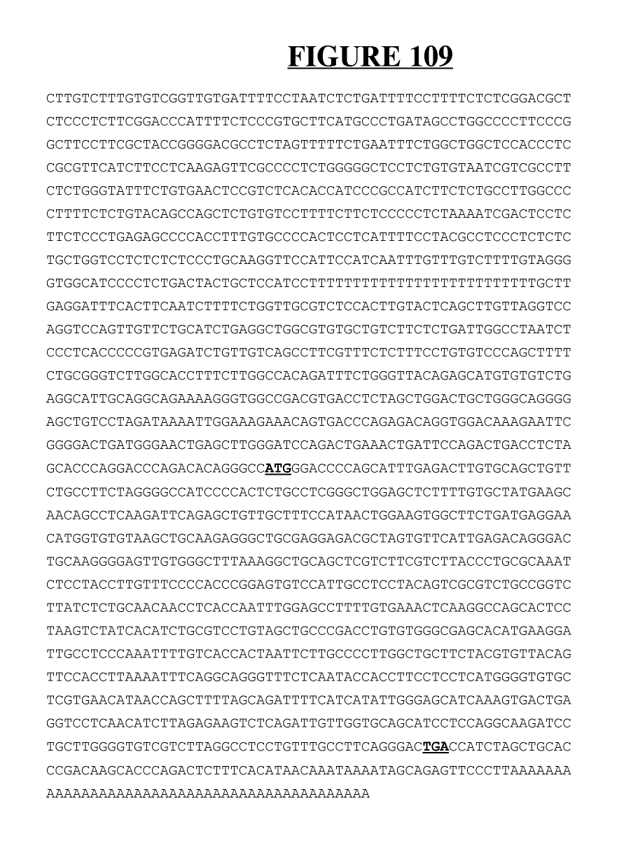

DNA sequencing of the clones isolated as described above gave the full-length DNA sequence for a full-length PRO6181 polypeptide (designated herein as DNA107698-2715 [FIG. 109, SEQ ID NO: 109]) and the derived protein sequence for that PRO6181 polypeptide.

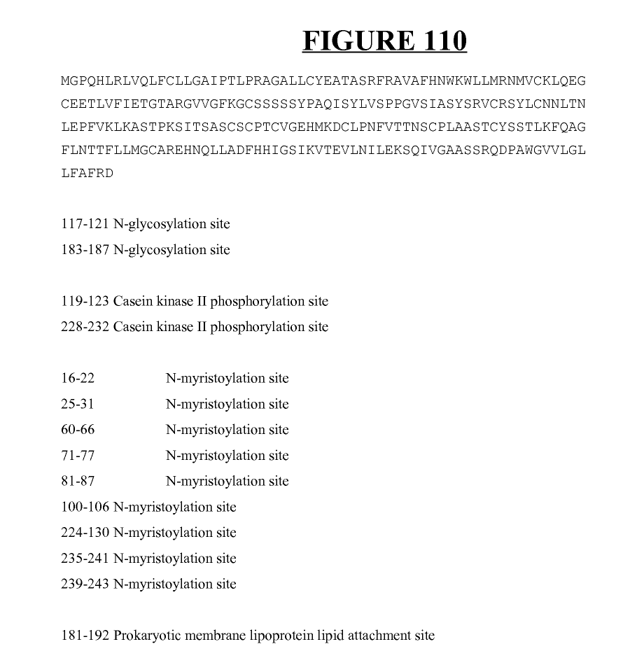

The full length clone identified above contained a single open reading frame with an apparent translational initiation site at nucleotide positions 986-988 and a stop signal at nucleotide positions 1724-1726 (FIG. 109, SEQ ID NO: 109). The predicted polypeptide precursor is 246 amino acids long, has a calculated molecular weight of approximately 26,773 daltons and an estimated pI of approximately 8.82. Analysis of the full-length PRO6181 sequence shown in FIG. 110 (SEQ ID NO:110) evidences the presence of a variety of important polypeptide domains as shown in FIG. 110, wherein the locations given for those important polypeptide domains are approximate as described above. Clone DNA107698-2715 has been deposited with ATCC on Aug. 3, 1999 and is assigned ATCC Deposit No. PTA-472.

An analysis of the Dayhoff database (version 35.45 SwissProt 35), using the ALIGN-2 sequence alignment analysis of the full-length sequence shown in FIG. 110 (SEQ ID NO: 110), evidenced sequence identity between the PRO6181 amino acid sequence and the following Dayhoff sequences: P_W67722; P_W79090; AC006276_{—}1; P_W26579; P_W79142; TTU15793_{—}1; YN9B_YEAST; 554056; AC005327_{—}1; and T01837.

### Example 56

### Isolation of cDNA Clones Encoding Human PRO6714 Polypeptides [UNQ2759]

DNA82358-2738 was identified by applying a proprietary signal sequence finding algorithm developed by Genentech, Inc. (South San Francisco, Calif.) upon ESTs as well as clustered and assembled EST fragments from public (e.g., Genbank) and/or private (LIFESEQ®, Incyte Pharmaceuticals, Inc., Palo Alto, Calif.) databases. The signal sequence algorithm computes a secretion signal score based on the character of the DNA nucleotides surrounding the first and optionally the second methionine codon(s) (ATG) at the 5′-end of the sequence or sequence fragment under consideration. The nucleotides following the first ATG must code for at least 35 unambiguous amino acids without any stop codons. If the first ATG has the required amino acids, the second is not examined. If neither meets the requirement, the candidate sequence is not scored. In order to determine whether the EST sequence contains an authentic signal sequence, the DNA and corresponding amino acid sequences surrounding the ATG codon are scored using a set of seven sensors (evaluation parameters) known to be associated with secretion signals.

Use of the above described signal sequence algorithm allowed identification of an EST cluster sequence from the LIFE SEQ® database, Incyte Pharmaceuticals, Palo Alto, Calif., designated herein as CLU15700. This EST cluster sequence was then compared to a variety of expressed sequence tag (EST) databases which included public EST databases (e.g., Genbank) and a proprietary EST DNA database (LIFESEQ®, Incyte Pharmaceuticals, Palo Alto, Calif.) to identify existing homologies. The homology search was performed using the computer program BLAST or BLAST2 (Altshul et al., Methods in Enzymology 266:460-480 (1996)). Those comparisons resulting in a BLAST score of 70 (or in some cases 90) or greater that did not encode known proteins were clustered and assembled into a consensus DNA sequence with the program “phrap” (Phil Green, University of Washington, Seattle, Wash.). The consensus sequence obtained therefrom is herein designated DNA73878.

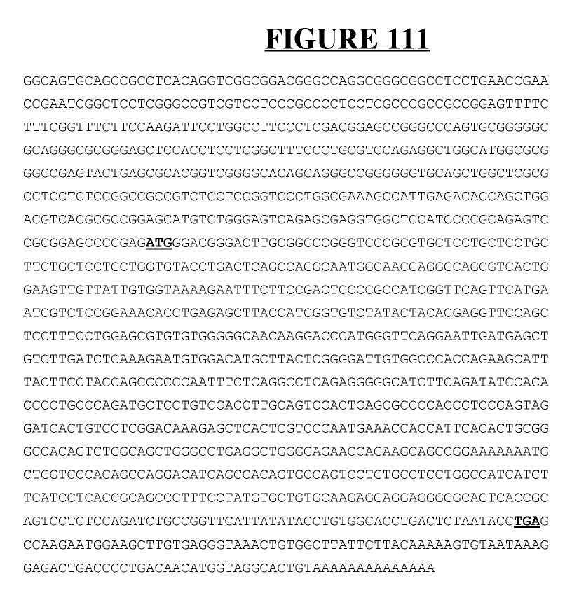

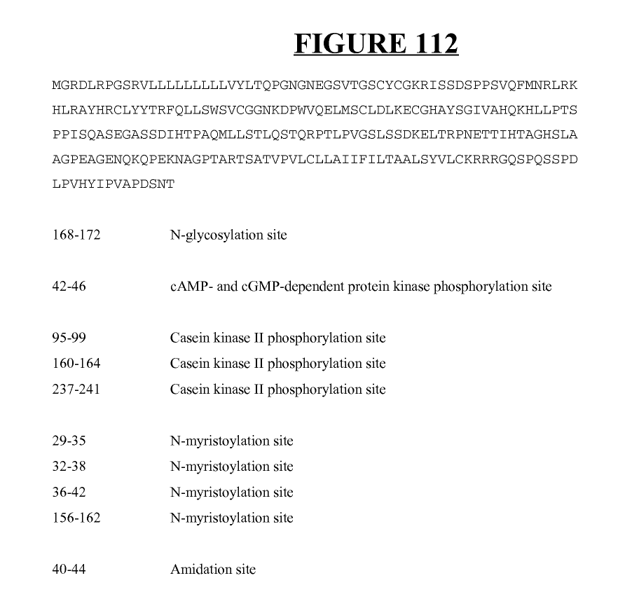

In light of an observed sequence homology between the DNA73878 sequence and an EST sequence encompassed within clone no. 3743689H1 from the LIFESEQ® database, Incyte Pharmaceuticals, Palo Alto, Calif., clone no. 3743689H1 was purchased and the cDNA insert was obtained and sequenced. It was found herein that that cDNA insert encoded a full-length protein. The sequence of this cDNA insert is shown in FIG. 111 and is herein designated as DNA82358-2738.

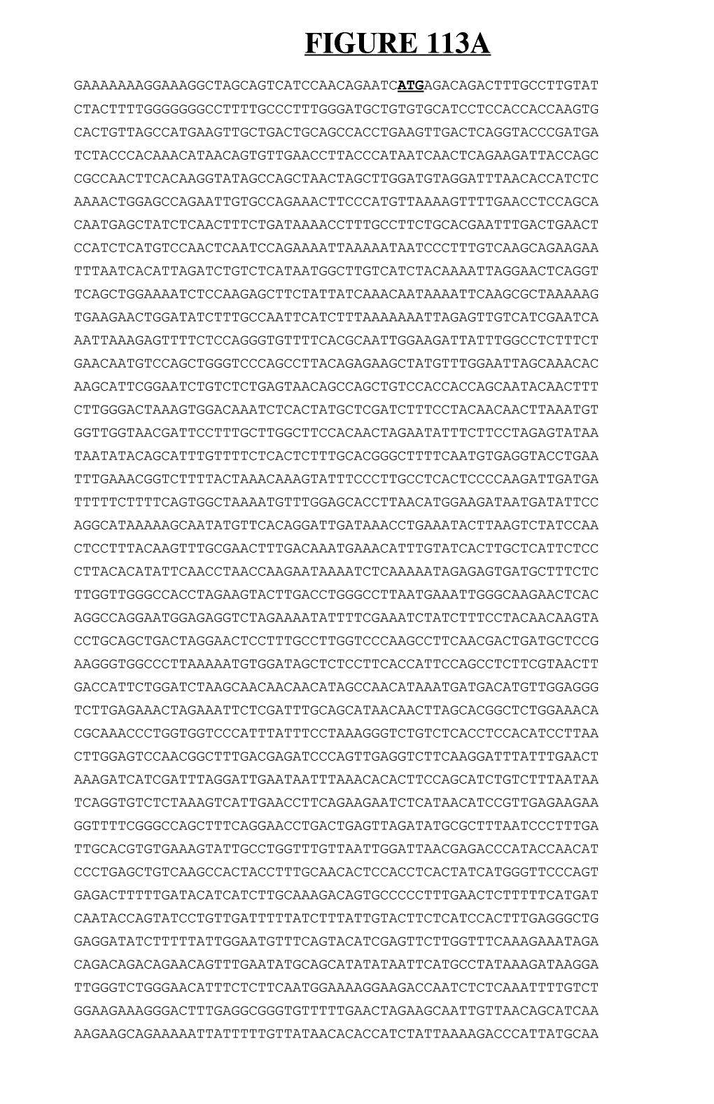

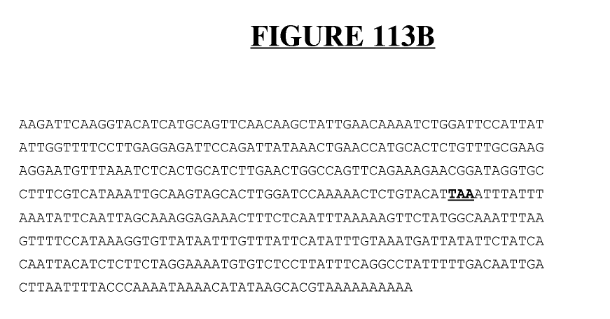

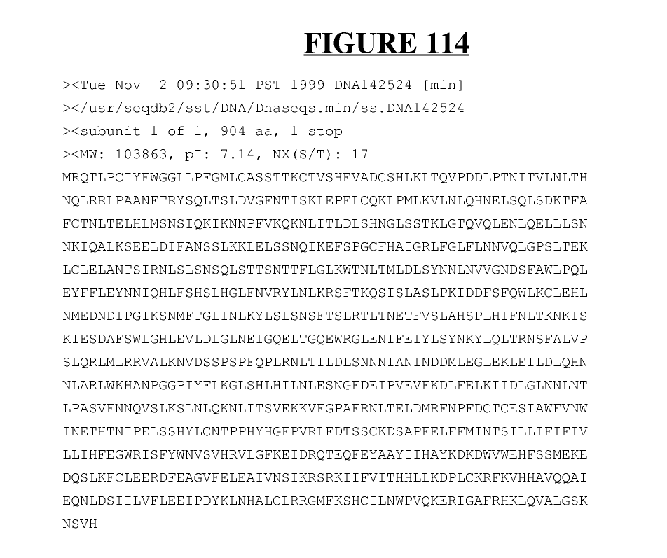

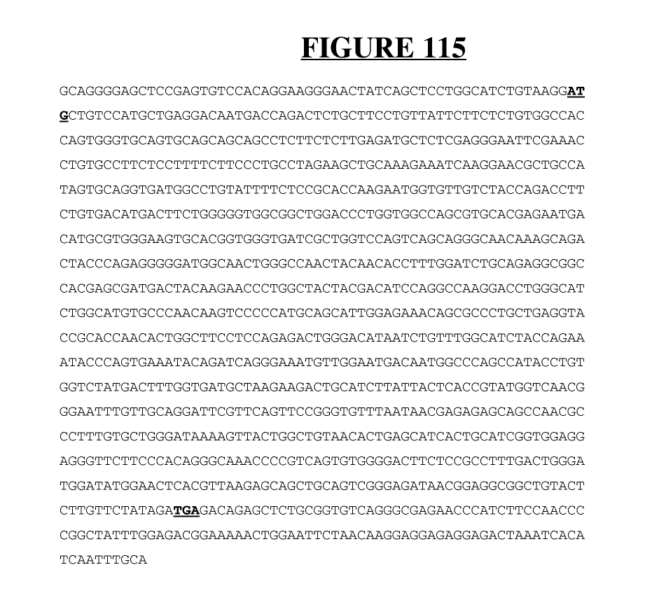

Clone DNA82358-2738 contains a single open reading frame with an apparent translational initiation site at nucleotide positions 435-437 and ending at the stop codon at nucleotide positions 1,197-1,199 (FIG. 111; SEQ ID NO:111). The predicted polypeptide precursor is 254 amino acids long (FIG. 112; SEQ ID NO:112). The full-length PRO6714 protein shown in FIG. 112 has an estimated molecular weight of about 27,579 daltons and a pI of about 9.14. Analysis of the full-length PRO6714 sequence shown in FIG. 112 (SEQ ID NO: 112) evidences the presence of a variety of important polypeptide domains as shown in FIG. 112, wherein the locations given for those important polypeptide domains are approximate as described above. Clone DNA82358-2738 has been deposited with ATCC on Aug. 10, 1999 and is assigned ATCC Deposit No. PTA-510.

An analysis of the Dayhoff database (version 35.45 SwissProt 35), using the ALIGN-2 sequence alignment analysis of the full-length sequence shown in FIG. 112 (SEQ ID NO: 112), evidenced sequence identity between the PRO6714 amino acid sequence and the following Dayhoff sequences: XLIMB1_{—}1; CELM70_{—}3; CELC01G8_{—}8; CEY47H₉B_{—}2; P_R70126; VIE1_MCMVS; ATT23J7_{—}20; EVU28134_{—}2; T02729; and I48201.

### Example 57

### Isolation of cDNA Clones Encoding Human PRO7179 Polypeptides [UNQ2789]

DNA108701-2749 was identified by applying a proprietary signal sequence finding algorithm developed by Genentech, Inc. (South San Francisco, Calif.) upon ESTs as well as clustered and assembled EST fragments from public (e.g., Genbank) and/or private (LIFESEQ®, Incyte Pharmaceuticals, Inc., Palo Alto, Calif.) databases. The signal sequence algorithm computes a secretion signal score based on the character of the DNA nucleotides surrounding the first and optionally the second methionine codon(s) (ATG) at the 5′-end of the sequence or sequence fragment under consideration. The nucleotides following the first ATG must code for at least 35 unambiguous amino acids without any stop codons. If the first ATG has the required amino acids, the second is not examined. If neither meets the requirement, the candidate sequence is not scored. In order to determine whether the EST sequence contains an authentic signal sequence, the DNA and corresponding amino acid sequences surrounding the ATG codon are scored using a set of seven sensors (evaluation parameters) known to be associated with secretion signals.

Use of the above described signal sequence algorithm allowed identification of an EST cluster sequence from the LIFESEQ®, Incyte Pharmaceuticals, Palo Alto, Calif. database, designated herein as CLU192050 designation. This EST cluster sequence was then compared to a variety of expressed sequence tag (EST) databases which included public EST databases (e.g., Genbank) and a proprietary EST DNA database (LIFESEQ®, Incyte Pharmaceuticals, Palo Alto, Calif.) to identify existing homologies. The homology search was performed using the computer program BLAST or BLAST2 (Altshul et al., Methods in Enzymology 266:460-480 (1996)). Those comparisons resulting in a BLAST score of 70 (or in some cases 90) or greater that did not encode known proteins were clustered and assembled into a consensus DNA sequence with the program “phrap” (Phil Green, University of Washington, Seattle, Wash.). The consensus sequence obtained therefrom is herein designated DNA91964.

In light of an observed sequence homology between the DNA91964 sequence and an EST sequence encompassed within clone no. 4049488 from the LIFESEQ®, Incyte Pharmaceuticals, Palo Alto, Calif. database, clone no. 4049488 was purchased and the cDNA insert was obtained and sequenced. It was found herein that that cDNA insert encoded a full-length protein. The sequence of this cDNA insert is shown in FIG. 115 and is herein designated as DNA108701-2749.

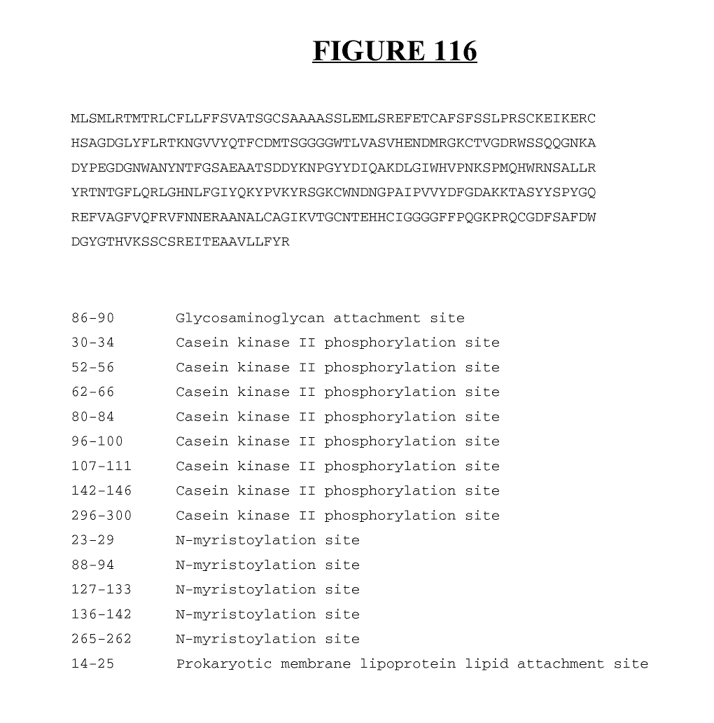

Clone DNA108701-2749 contains a single open reading frame with an apparent translational initiation site at nucleotide positions 59-61 and ending at the stop codon at nucleotide positions 1034-1036 (FIG. 115; SEQ ID NO:115). The predicted polypeptide precursor is 325 amino acids long (FIG. 116; SEQ ID NO:116). The full-length PRO7179 protein shown in FIG. 116 has an estimated molecular weight of about 36212 daltons and a pI of about 8.68. Analysis of the full-length PRO7179 sequence shown in FIG. 116 (SEQ ID NO: 116) evidences the presence of a variety of important polypeptide domains as shown in FIG. 116, wherein the locations given for those important polypeptide domains are approximate as described above. Clone DNA108701-2749 has been deposited with ATCC on Aug. 17, 1999 and is assigned ATCC Deposit No. PTA-554.

An analysis of the Dayhoff database (version 35.45 SwissProt 35), using the ALIGN-2 sequence alignment analysis of the full-length sequence shown in FIG. 116 (SEQ ID NO: 116), evidenced sequence identity between the PRO7179 amino acid sequence and the following Dayhoff sequences: P_Y02655; HGS_RQ155; JE0328; XLU86699_{—}1; S49589; HGS_RQ307; P_Y02807; FIBA_PARPA; P_R82243; FIBB_HUMAN.

### Example 58

### Isolation of cDNA Clones Encoding Human PRO7476 Polypeptides [UNQ2976]

A search was performed using the computer program BLAST or BLAST2 [Altschul et al., Methods in Enzymology, 266:460-480 (1996)] for cytokine/growth factor homologs. A 94.5 KB piece was found to contain exons encoding growth factor homologs, however, this piece was broken up by large introns. The introns were removed by a computer algorithm. Based on the DNA102863 consensus sequence, oligonucleotides were synthesized: 1) to identify by PCR a cDNA library that contained the sequence of interest, and 2) for use as probes to isolate a clone of the full-length coding sequence for PRO7476. Forward and reverse PCR primers generally range from 20 to 30 nucleotides and are often designed to give a PCR product of about 100-1000 by in length. The probe sequences are typically 40-55 by in length. In some cases, additional oligonucleotides are synthesized when the consensus sequence is greater than about 1-1.5 kbp. In order to screen several libraries for a full-length clone, DNA from the libraries was screened by PCR amplification, as per Ausubel et al., Current Protocols in Molecular Biology, supra, with the PCR primer pair. A positive library was then used to isolate clones encoding the gene of interest using the probe oligonucleotide and one of the primer pairs.

PCR primers (forward and reverse) were synthesized:

| | |
|---|---|
| forward PCR primer: | |
| 5′-ATGCAGCTCCCACTGGCCCTG-3′ | (SEQ ID NO: 249) |
| reverse PCR primer: | |
| 5′-CTAGTAGGCGTTCTCCAGCTCGGCCTG-3′ | (SEQ ID NO: 250) |

Additionally, a synthetic oligonucleotide hybridization probe was constructed from the consensus DNA102863 sequence which had the following nucleotide sequence:

hybridization probe:
(SEQ ID NO: 251)
5′-CTTCCGCTGCATCCCCGACCGCTACCGCGCGCAGCGCGTG-3′

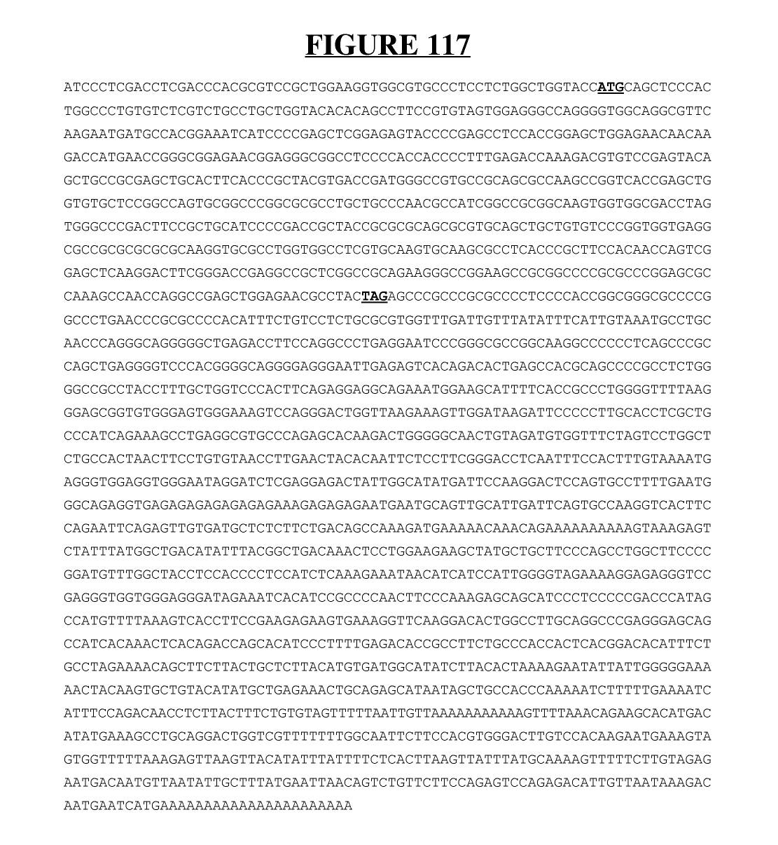

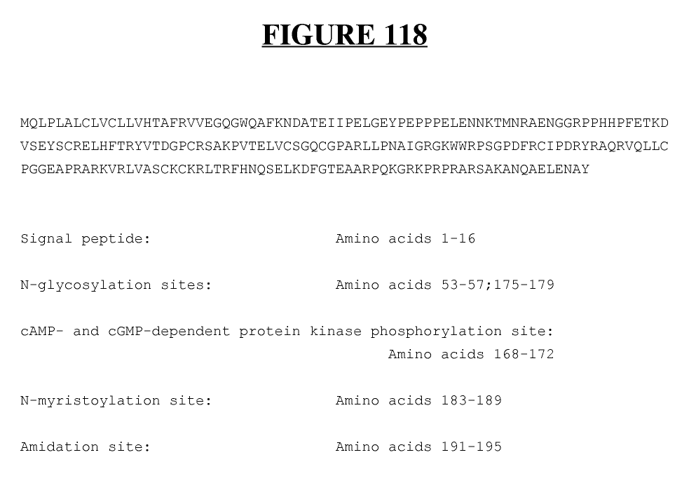

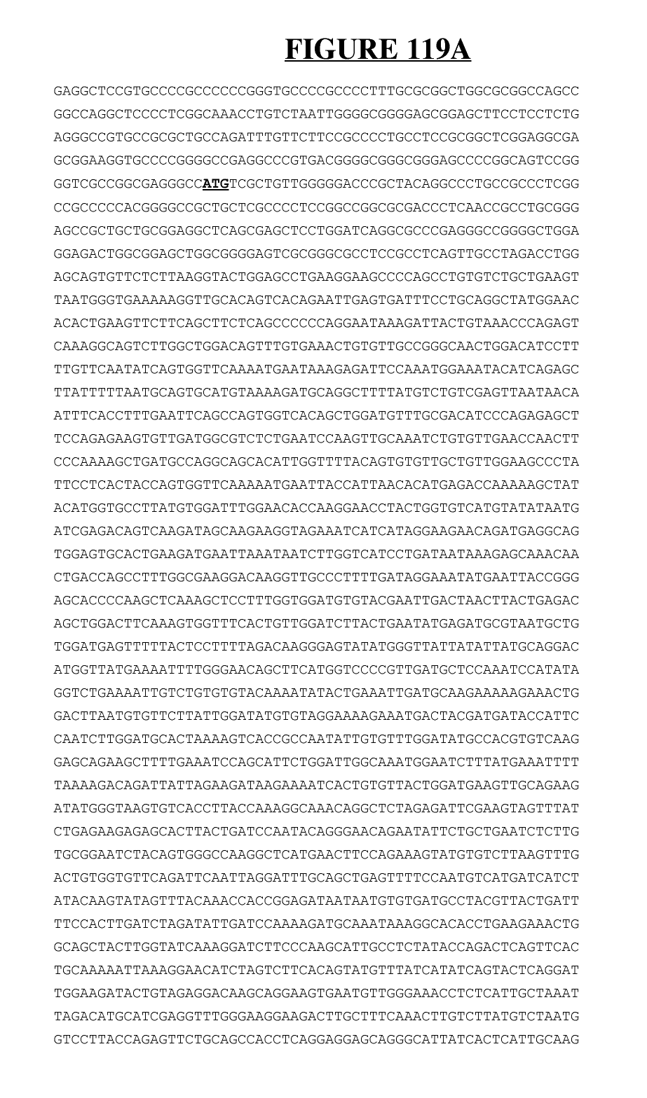

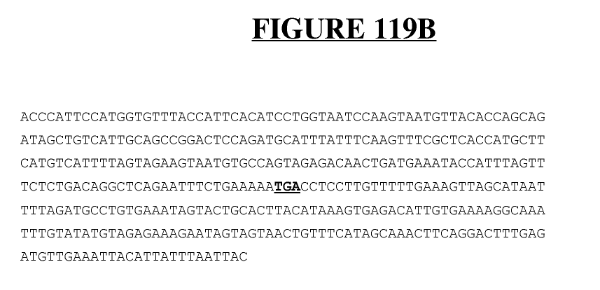

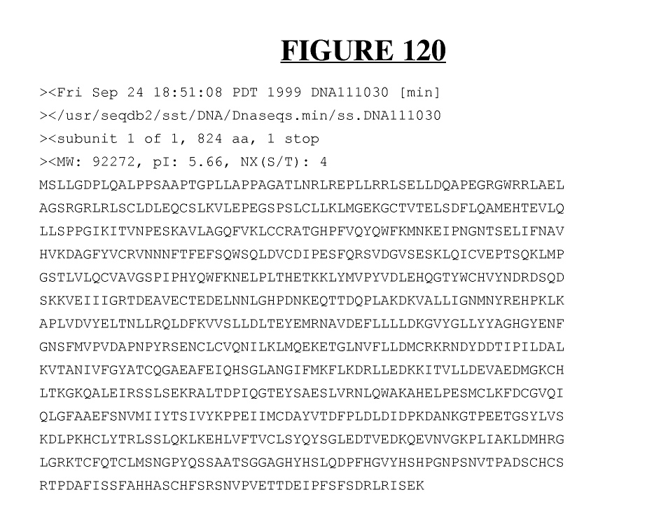

DNA sequencing of the clones isolated as described above gave the full-length DNA sequence for a full-length PRO7476 polypeptide (designated herein as DNA115253-2757 [FIG. 117, SEQ ID NO: 117]) and the derived protein sequence for that PRO7476 polypeptide.

The full length clone identified above contained a single open reading frame with an apparent translational initiation site at nucleotide positions 62-64 and a stop signal at nucleotide positions 701-703 (FIG. 117, SEQ ID NO: 117). The predicted polypeptide precursor is 213 amino acids long, has a calculated molecular weight of approximately 24,031 daltons and an estimated pI of approximately 9.59. Analysis of the full-length PRO7476 sequence shown in FIG. 118 (SEQ ID NO: 118) evidences the presence of a variety of important polypeptide domains as shown in FIG. 118, wherein the locations given for those important polypeptide domains are approximate as described above. Clone DNA115253-2757 has been deposited with ATCC on Aug. 31, 1999 and is assigned ATCC Deposit No. PTA-612.

An analysis of the Dayhoff database (version 35.45 SwissProt 35), using the ALIGN-2 sequence alignment analysis of the full-length sequence shown in FIG. 118 (SEQ ID NO: 118), evidenced sequence identity between the PRO7476 amino acid sequence and the following Dayhoff sequences: P_W58704; P_W95711; P_WO9408; P_Y12009; T08710; P_W44090; P_W27654; P_Y03225; LSHB_MELGA; AB011030_{—}1.

### Example 59

### Isolation of cDNA Clones Encoding Human PRO19814 Polypeptides [UNQ5923]

The extracellular domain (ECD) sequences (including the secretion signal sequence, if any) from about 950 known secreted proteins from the Swiss-Prot public database were used to search EST databases. The EST databases included LIFESEQ7, Incyte Pharmaceuticals, Palo Alto, Calif.). The search was performed using the computer program BLAST or BLAST2 [Altschul et al., Methods in Enzymology, 266:460-480 (1996)] as a comparison of the ECD protein sequences to a 6 frame translation of the EST sequences. Those comparisons resulting in a BLAST score of 70 (or in some cases, 90) or greater that did not encode known proteins were clustered and assembled into consensus DNA sequences with the program “phrap” (Phil Green, University of Washington, Seattle, Wash.).

A consensus DNA sequence was assembled relative to other EST sequences using phrap as described above. This consensus sequence is herein designated DNA47457. In some cases, the consensus sequence derives from an intermediate consensus DNA sequence which was extended using repeated cycles of BLAST and phrap to extend that intermediate consensus sequence as far as possible using the sources of EST sequences discussed above.

Based on the DNA47457 consensus sequence oligonucleotides were synthesized: 1) to identify by PCR a cDNA library that contained the sequence of interest, and 2) for use as probes to isolate a clone of the full-length coding sequence for PRO19814. Forward and reverse PCR primers generally range from 20 to 30 nucleotides and are often designed to give a PCR product of about 100-1000 by in length. The probe sequences are typically 40-55 by in length. In some cases, additional oligonucleotides are synthesized when the consensus sequence is greater than about 1-1.5 kbp. In order to screen several libraries for a full-length clone, DNA from the libraries was screened by PCR amplification, as per Ausubel et al., Current Protocols in Molecular Biology, supra, with the PCR primer pair. A positive library was then used to isolate clones encoding the gene of interest using the probe oligonucleotide and one of the primer pairs.PCR primers (forward and reverse) were synthesized:

| | |
|---|---|
| forward PCR primer | |
| 5′-CATCGTGTGTCGTGCCACCAAC-3′ | (SEQ ID NO: 252) |
| reverse PCR primer | |
| 5′-CTCTGGCCATTCTCCACGTCACC-3′ | (SEQ ID NO: 253) |

Additionally, a synthetic oligonucleotide hybridization probe was constructed from the consensus DNA47457 sequence which had the following nucleotide sequence

hybridization probe
(SEQ ID NO: 254)
5′-GGAAAGGAGACGTCGGTCACCATTGACATCCAGCACCCTCCAC-3′

RNA for construction of the cDNA libraries was isolated from human oligo dT from mixed tissue. The cDNA libraries used to isolate the cDNA clones were constructed by standard methods using commercially available reagents such as those from Invitrogen, San Diego, Calif. The cDNA was primed with oligo dT containing a NotI site, linked with blunt to SalI hemikinased adaptors, cleaved with NotI, sized appropriately by gel electrophoresis, and cloned in a defined orientation into a suitable cloning vector (such as pRKB or pRKD; pRK5B is a precursor of pRK5D that does not contain the SfiI site; see, Holmes et al., Science, 253:1278-1280 (1991)) in the unique XhoI and NotI sites.

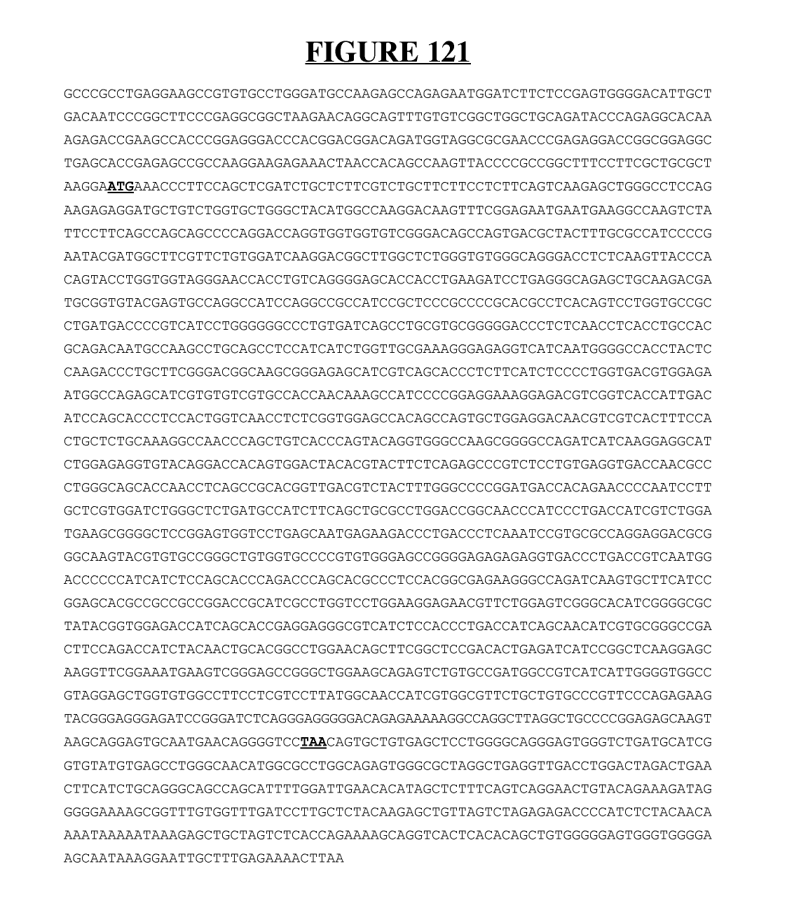

DNA sequencing of the clones isolated as described above gave the full-length DNA sequence for a full-length PRO19814 polypeptide (designated herein as DNA148004-2882 [FIG. 121, SEQ ID NO: 121]) and the derived protein sequence for that PRO19814 polypeptide.

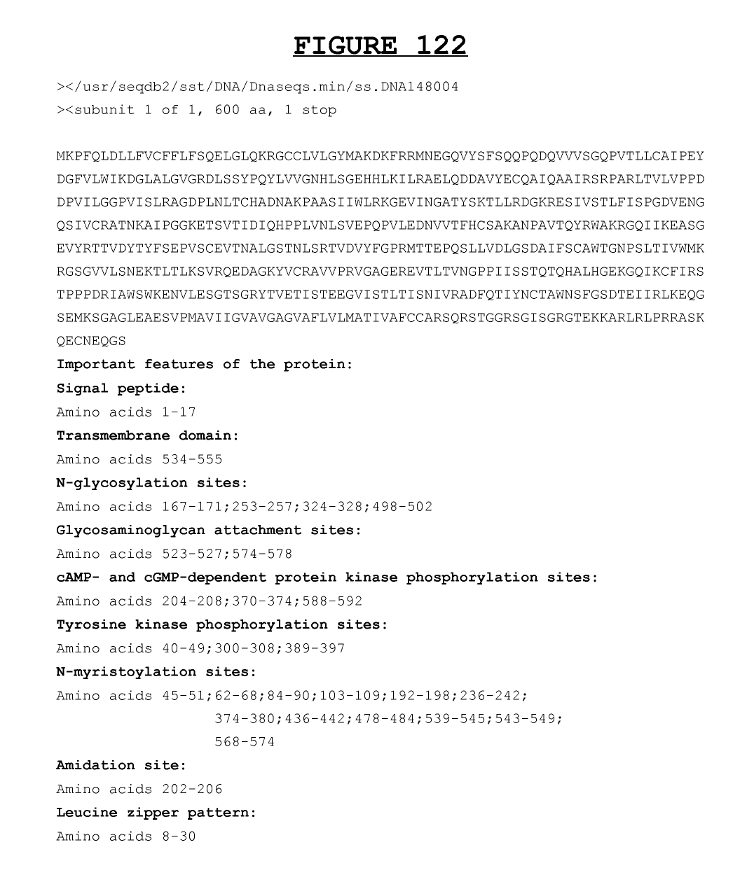

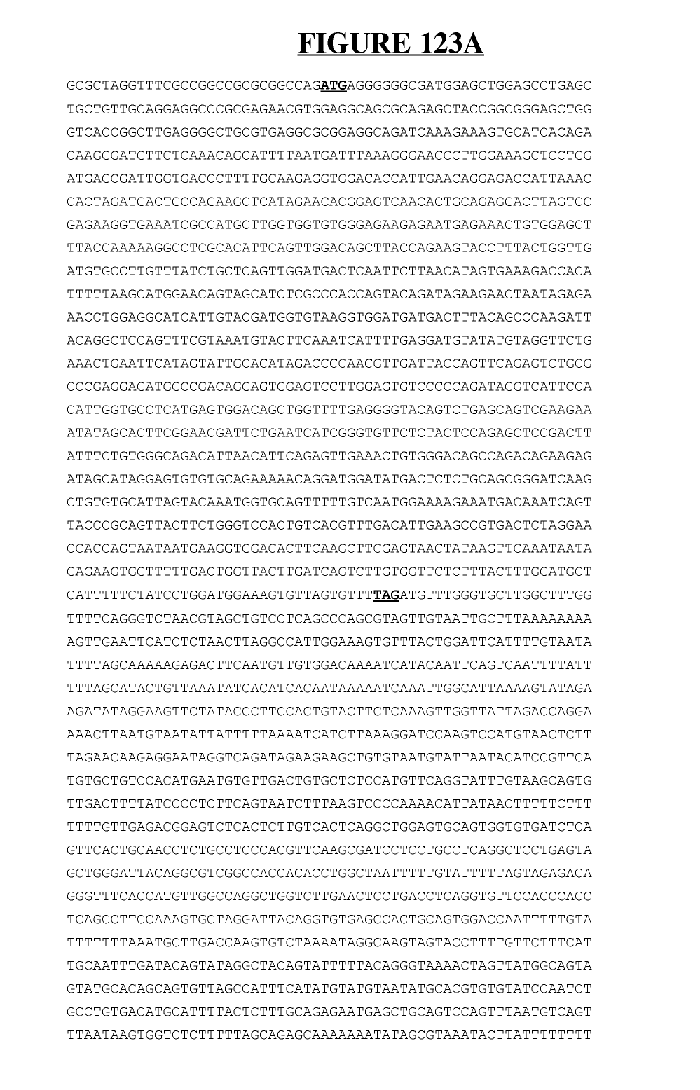

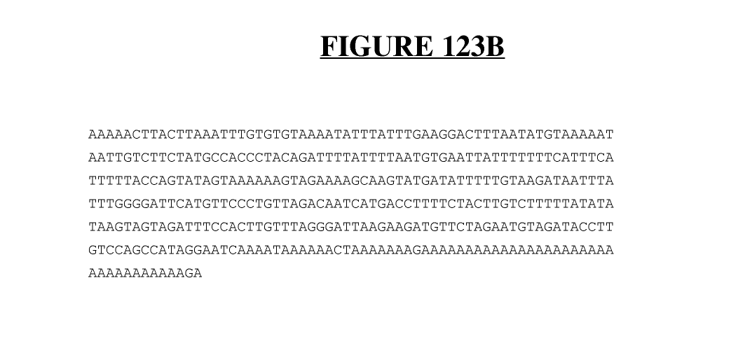

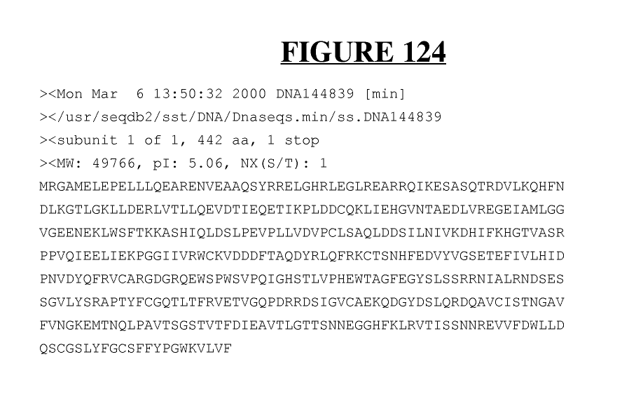

The full length clone identified above contained a single open reading frame with an apparent translational initiation site at nucleotide positions 302-304 and a stop signal at nucleotide positions 2101-2103 (FIG. 121, SEQ ID NO:121). The predicted polypeptide precursor is 600 amino acids long, has a calculated molecular weight of approximately 65,308 daltons and an estimated pI of approximately 8.35. Analysis of the full-length PRO19814 sequence shown in FIG. 122 (SEQ ID NO:122) evidences the presence of a variety of important polypeptide domains as shown in FIG. 122, wherein the locations given for those important polypeptide domains are approximate as described above. Clone DNA148004-2882 has been deposited with ATCC on Apr. 25, 2000 and is assigned ATCC deposit no. PTA-1779.

### Example 60

### Isolation of cDNA Clones Encoding Human PRO20088 Polypeptides [UNQ6077]

The extracellular domain (ECD) sequences (including the secretion signal sequence, if any) from about 950 known secreted proteins from the Swiss-Prot public database were used to search EST databases. The EST databases included a proprietary EST database (LIFESEQ®, Incyte Pharmaceuticals, Palo Alto, Calif.). The search was performed using the computer program BLAST or BLAST2 [Altschul et al., Methods in Enzymology, 266:460-480 (1996)] as a comparison of the ECD protein sequences to a 6 frame translation of the EST sequences. Those comparisons resulting in a BLAST score of 70 (or in some cases, 90) or greater that did not encode known proteins were clustered and assembled into consensus DNA sequences with the program “phrap” (Phil Green, University of Washington, Seattle, Wash.).

A consensus DNA sequence was assembled relative to other EST sequences using phrap as described above. This consensus sequence is herein designated DNA90859. In some cases, the DNA90859 consensus sequence derives from an intermediate consensus DNA sequence which was extended using repeated cycles of BLAST and phrap to extend that intermediate consensus sequence as far as possible using the sources of EST sequences discussed above.

Based on the DNA90859 consensus sequence oligonucleotides were synthesized: 1) to identify by PCR a cDNA library that contained the sequence of interest, and 2) for use as probes to isolate a clone of the full-length coding sequence for PRO20088. Forward and reverse PCR primers generally range from 20 to 30 nucleotides and are often designed to give a PCR product of about 100-1000 by in length. The probe sequences are typically 40-55 by in length. In some cases, additional oligonucleotides are synthesized when the consensus sequence is greater than about 1-1.5 kbp. In order to screen several libraries for a full-length clone, DNA from the libraries was screened by Flip PCR amplification, as per Schanke et al., BioTechniques, 16:414-416 (1994), with the PCR primer pair. A positive library was then used to isolate clones encoding the gene of interest using the probe oligonucleotide and one of the primer pairs.

PCR primers (forward and reverse) were synthesized:

| | |
|---|---|
| forward PCR primer | |
| 5′-GCTGCTCGTGCTCCGGCTG-3′ | (SEQ ID NO: 255) |
| reverse PCR primer | |
| 5′-CACAAAACGACAATCCGGGCCTG-3′ | (SEQ ID NO: 256) |

Additionally, a synthetic oligonucleotide hybridization probe was constructed from the consensus DNA90859 sequence which had the following nucleotide sequence

hybridization probe
(SEQ ID NO: 257)
5′-GCACAAACTCTGCGCGGACGACGAATGCAGCATGTTAATGTAC-3′

RNA for construction of the cDNA libraries was isolated from human tissue. The cDNA libraries used to isolate the cDNA clones were constructed by standard methods using commercially available reagents such as those from Invitrogen, San Diego, Calif. The cDNA was primed with oligo dT containing a NotI site, linked with blunt to SalI hemikinased adaptors, cleaved with NotI, sized appropriately by gel electrophoresis, and cloned in a defined orientation into a suitable cloning vector (such as pRKB or pRKD; pRK5B is a precursor of pRK5D that does not contain the SfiI site; see, Holmes et al., Science, 253:1278-1280 (1991)) in the unique XhoI and NotI sites.

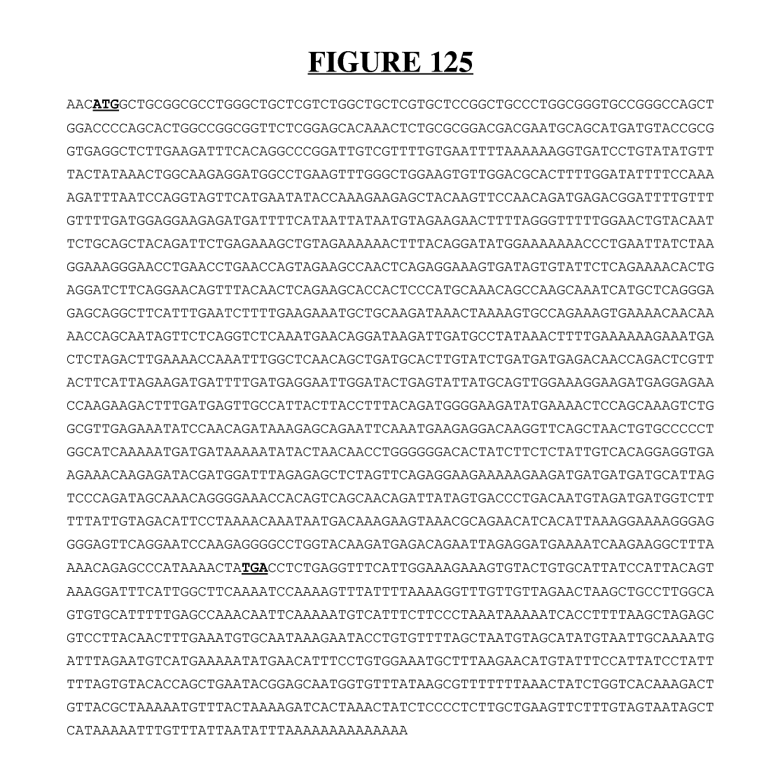

DNA sequencing of the clones isolated as described above gave the full-length DNA sequence for a full-length PRO20088 polypeptide (designated herein as DNA150157-2898 [FIG. 125, SEQ ID NO: 125]) and the derived protein sequence for that PRO20088 polypeptide.

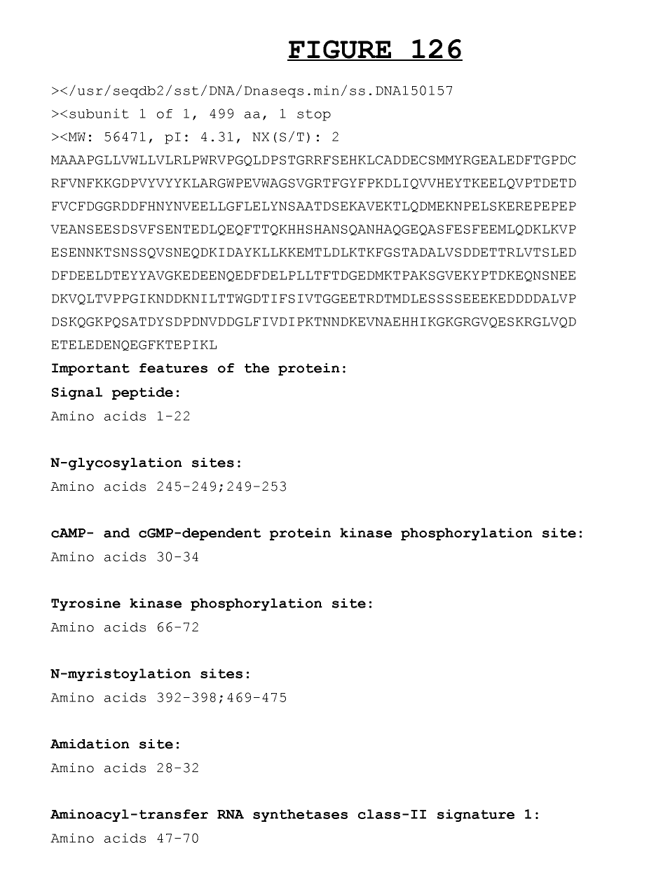

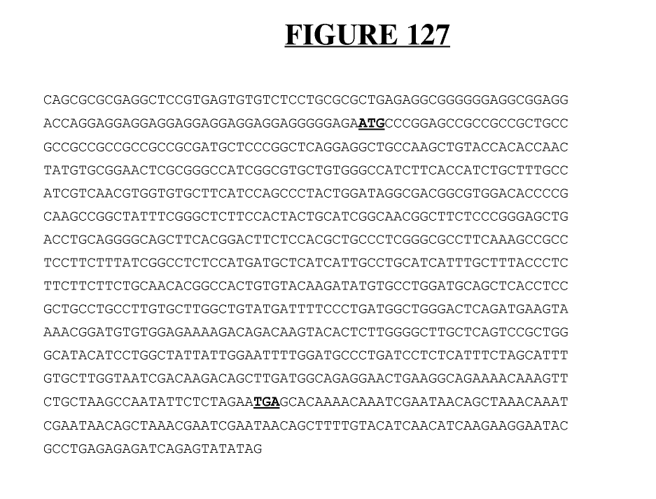

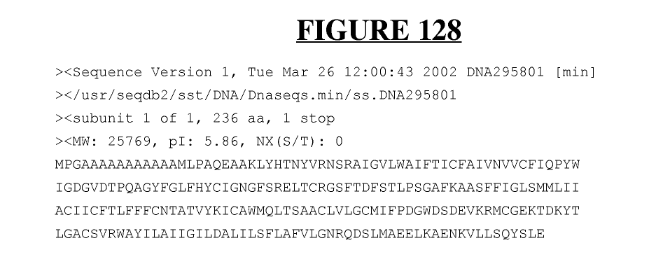

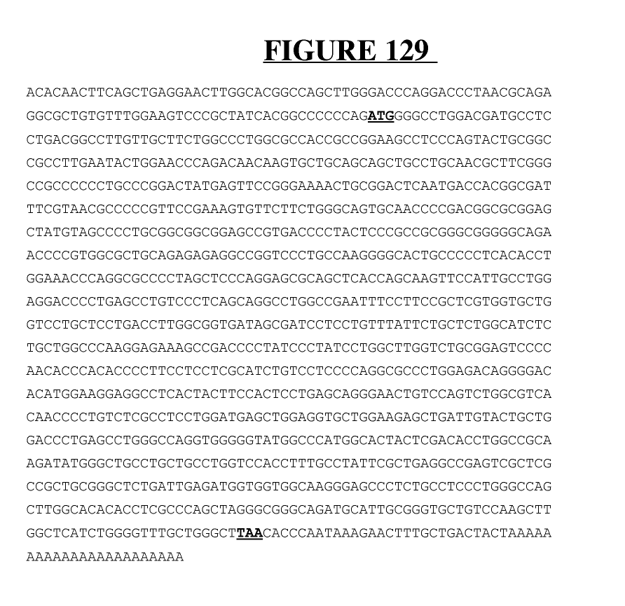

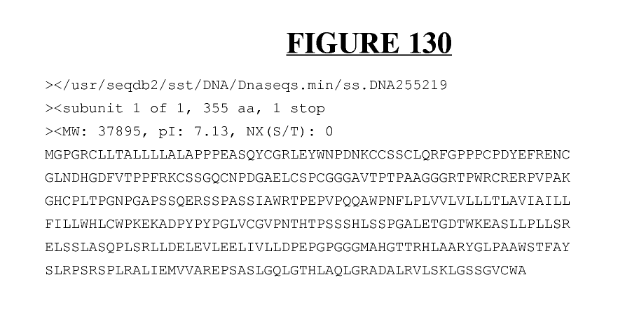

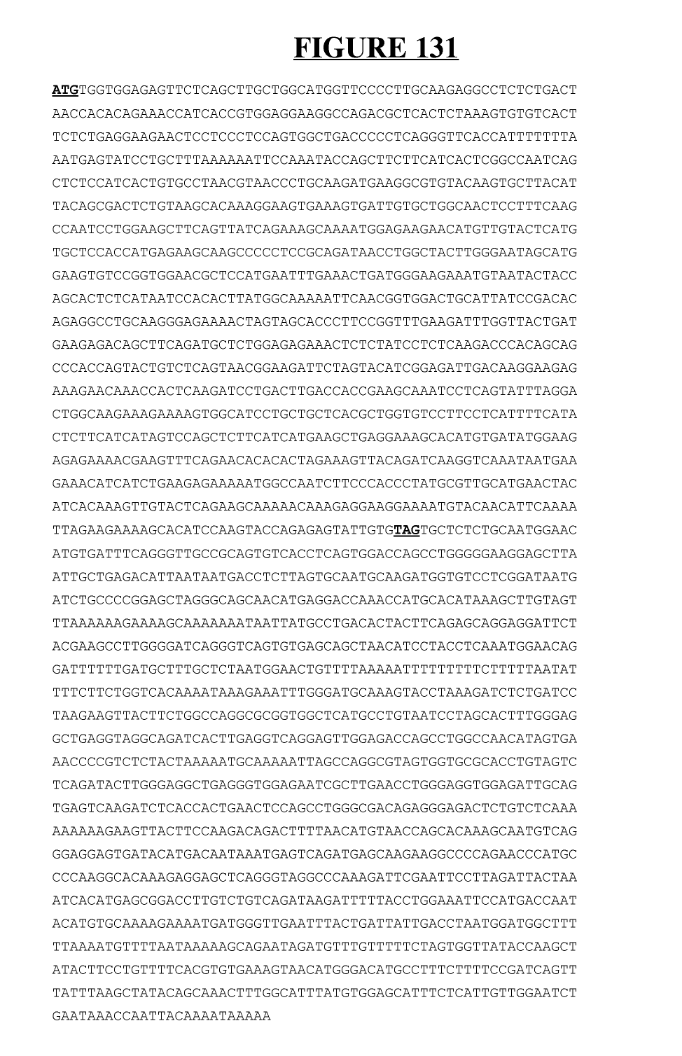

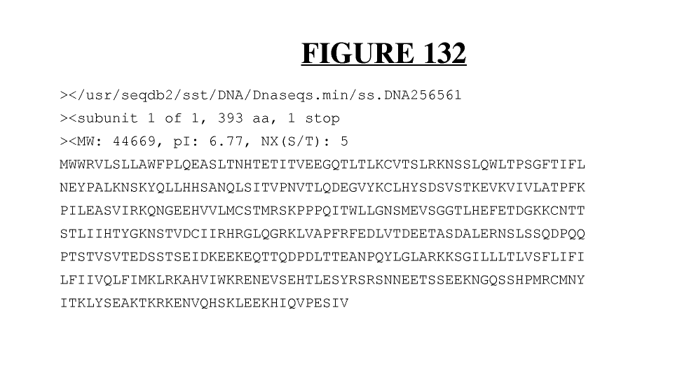

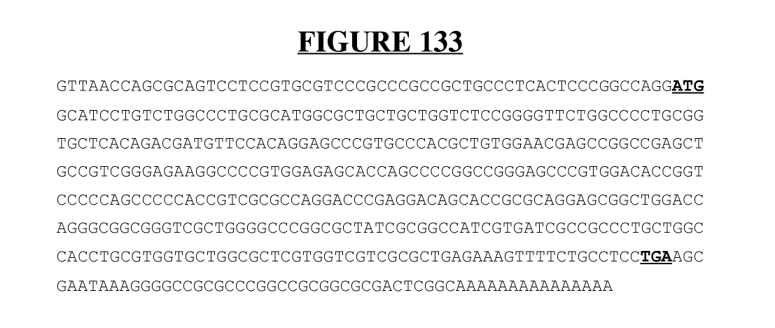

The full length clone identified above contained a single open reading frame with an apparent translational initiation site at nucleotide positions 4-6 and a stop signal at nucleotide positions 1501-1503 (FIG. 125, SEQ ID NO: 125). The predicted polypeptide precursor is 499 amino acids long, has a calculated molecular weight of approximately 56,471 daltons and an estimated pI of approximately 4.31. Analysis of the full-length PRO20088 sequence shown in FIG. 126 (SEQ ID NO: 126) evidences the presence of a variety of important polypeptide domains as shown in FIG. 126, wherein the locations given for those important polypeptide domains are approximate as described above. Clone DNA150157-2898 has been deposited with ATCC on Apr. 25, 2000 and is assigned ATCC Deposit No. PTA-1777.

An analysis of the Dayhoff database (version 35.45 SwissProt 35), using the ALIGN-2 sequence alignment analysis of the full-length sequence shown in FIG. 126 (SEQ ID NO: 126), evidenced sequence identity between the PRO20088 amino acid sequence and the following Dayhoff sequences: P_Y24788; A71623; NM_{—}006533_{—}1; MIA_HUMAN; P_R69811; MMU85612_{—}1; GEN14164; P_W03627; AF148805_{—}6; and AF206632_{—}1.

### Example 61

### Isolation of cDNA Clones Encoding Human PRO1757 Polypeptides [UNQ830]

Use of the signal sequence algorithm described in Example 3 above allowed identification of three EST sequences from the Incyte database, designated Incyte EST clones no. 2007947, 2014962 and 1912034. These EST sequences were then clustered and assembled into a consensus DNA sequence with the program “phrap” (Phil Green, University of Washington, Seattle, Wash.). The consensus sequence obtained therefrom is herein designated as DNA56054.

In light of the sequence homology between the DNA56054 sequence and a sequence contained within the Incyte EST clone no. 2007947, the Incyte EST clone no. 2007947 was purchased and the cDNA insert was obtained and sequenced. The sequence of this cDNA is herein designated as DNA76398-1699.

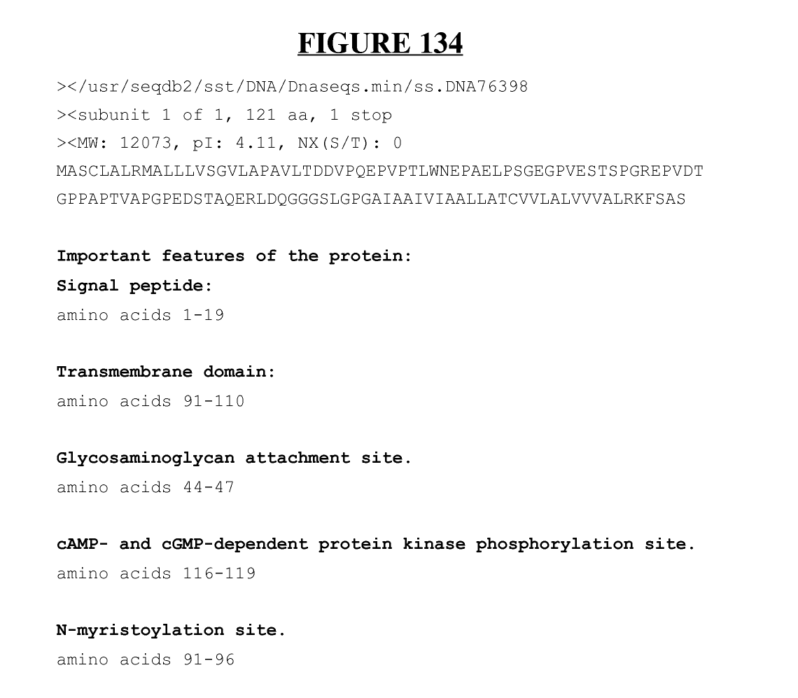

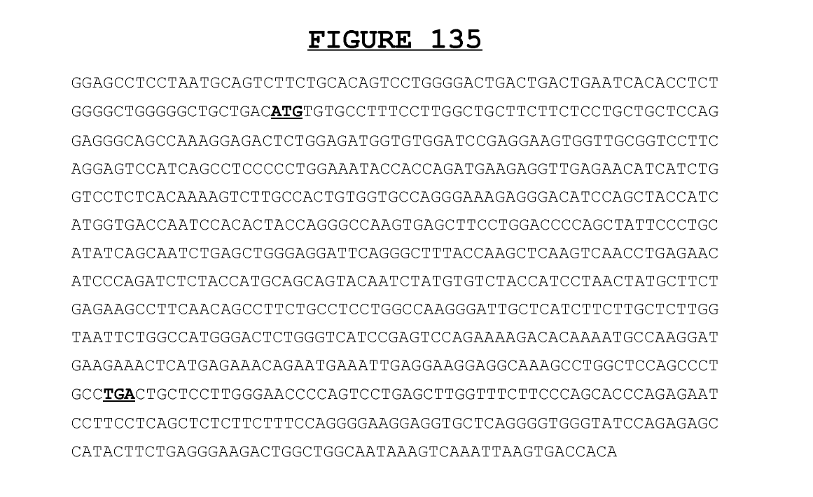

Clone DNA76398-1699 contains a single open reading frame with an apparent translational initiation site at nucleotide positions 59-61 and ending at the stop codon at nucleotide positions 422-424 (FIG. 133; SEQ ID NO:133). The predicted polypeptide precursor is 121 amino acids long (FIG. 134; SEQ ID NO:134). The full-length PRO1757 protein shown in FIG. 134 has an estimated molecular weight of about 12,073 daltons and a pI of about 4.11. Analysis of the full-length PRO1757 sequence shown in FIG. 134 (SEQ ID NO:134) evidences the presence of the following: a signal peptide from about amino acid 1 to about amino acid 19, a transmembrane domain from about amino acid 91 to about amino acid 110, a glycosaminoglycan attachment site from about amino acid 44 to about amino acid 47, a cAMP- and cGMP-dependent protein kinase phosphorylation site from about amino acid 116 to about amino acid 119 and a potential N-myristolation site from about amino acid 91 to about amino acid 96. Clone DNA76398-1699 has been deposited with ATCC on Nov. 17, 1998 and is assigned ATCC deposit no. 203474.

An analysis of the Dayhoff database (version 35.45 SwissProt 35), using a WU-BLAST2 sequence alignment analysis of the full-length sequence shown in FIG. 134 (SEQ ID NO:134), evidenced significant homology between the PRO1757 amino acid sequence and the following Dayhoff sequences: JQ0964, COLL_HSVS7, HSU70136_{—}1, AF003473_{—}1, D89728_{—}1, MTF1_MOUSE, AF029777_{—}1, HSU88153_{—}1 and P_W05321.

### Example 62

### Isolation of cDNA Clones Encoding Human PRO4421 Polypeptides [UNQ1938]

DNA96879-2619 was identified by applying a proprietary signal sequence finding algorithm developed by Genentech, Inc. (South San Francisco, Calif.) upon ESTs as well as clustered and assembled EST fragments from public (e.g., GenBank) and/or private (LIFESEQ®, Incyte Pharmaceuticals, Inc., Palo Alto, Calif.) databases. The signal sequence algorithm computes a secretion signal score based on the character of the DNA nucleotides surrounding the first and optionally the second methionine codon(s) (ATG) at the 5′-end of the sequence or sequence fragment under consideration. The nucleotides following the first ATG must code for at least 35 unambiguous amino acids without any stop codons. If the first ATG has the required amino acids, the second is not examined. If neither meets the requirement, the candidate sequence is not scored. In order to determine whether the EST sequence contains an authentic signal sequence, the DNA and corresponding amino acid sequences surrounding the ATG codon are scored using a set of seven sensors (evaluation parameters) known to be associated with secretion signals.

Use of the above described signal sequence algorithm allowed identification of an EST cluster sequence from the Incyte database. This EST cluster sequence was then compared to a variety of expressed sequence tag (EST) databases which included public EST databases (e.g., GenBank) and a proprietary EST DNA database (LIFESEQ®, Incyte Pharmaceuticals, Palo Alto, Calif.) to identify existing homologies. The homology search was performed using the computer program BLAST or BLAST2 (Altshul et al., Methods in Enzymology 266:460-480 (1996)). Those comparisons resulting in a BLAST score of 70 (or in some cases 90) or greater that did not encode known proteins were clustered and assembled into a consensus DNA sequence with the program “phrap” (Phil Green, University of Washington, Seattle, Wash. The consensus sequence obtained therefrom is herein designated DNA80133. Within the assembly an EST in the Genentech database was identified. In light of this sequence, DNA96879-2619 was identified and sequenced.

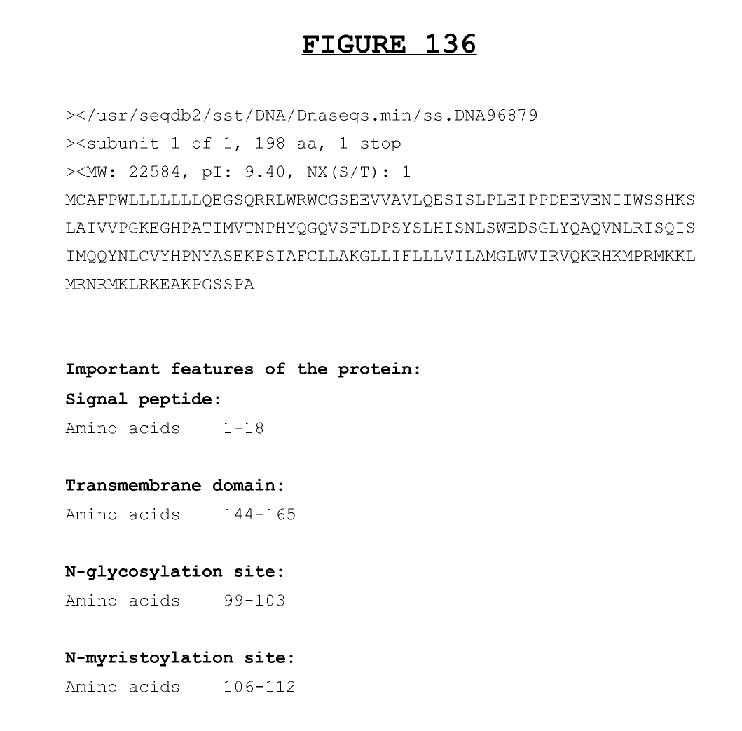

The full length clone shown in FIG. 135 contained a single open reading frame with an apparent translational initiation site at nucleotide positions 81-83 and ending at the stop codon found at nucleotide positions 675-677 (FIG. 135; SEQ ID NO:135). The predicted polypeptide precursor (FIG. 136, SEQ ID NO:136) is 198 amino acids long. PRO4421 has a calculated molecular weight of approximately 22584 daltons and an estimated pI of approximately 9.4.

An analysis of the Dayhoff database (version 35.45 SwissProt 35), using a WU-BLAST2 sequence alignment analysis of the full-length sequence shown in FIG. 136 (SEQ ID NO:136), revealed homology between the PRO4421 amino acid sequence and the following Dayhoff sequences (sequences and related text incorporated herein): HSU82988_{—}1, HGS_B476, HSAJ3324_{—}1, HSU96627_{—}1, HUMLY9_{—}1, AF043445_{—}1, LY9_MOUSE, AC005626_{—}1, P_R71478 and CD86_HUMAN.

Clone DNA96879-2619 (UNQ1938), designated as DNA96879-2619 was deposited with the ATCC on Apr. 27, 1999 and is assigned ATCC deposit no. 203967.

### Example 63

### Isolation of cDNA Clones Encoding Human PRO9903 Polypeptides [UNQ3071]

DNA119596-2797 was identified by applying a proprietary signal sequence finding algorithm developed by Genentech, Inc. (South San Francisco, Calif.) upon ESTs as well as clustered and assembled EST fragments from public (e.g., GenBank) and/or private (LIFESEQ, Incyte Pharmaceuticals, Inc., Palo Alto, Calif.) databases. The signal sequence algorithm computes a secretion signal score based on the character of the DNA nucleotides surrounding the first and optionally the second methionine codon(s) (ATG) at the 5′-end of the sequence or sequence fragment under consideration. The nucleotides following the first ATG must code for at least 35 unambiguous amino acids without any stop codons. If the first ATG has the required amino acids, the second is not examined. If neither meets the requirement, the candidate sequence is not scored. In order to determine whether the EST sequence contains an authentic signal sequence, the DNA and corresponding amino acid sequences surrounding the ATG codon are scored using a set of seven sensors (evaluation parameters) known to be associated with secretion signals.

Use of the above described signal sequence algorithm allowed identification of an EST cluster sequence from the LIFESEQ database, designated herein as CLU67175. This EST cluster sequence was then compared to a variety of expressed sequence tag (EST) databases which included public EST databases (e.g., GenBank) and a proprietary EST DNA database (LIFESEQ™, Incyte Pharmaceuticals, Palo Alto, Calif.) to identify existing homologies. The homology search was performed using the computer program BLAST or BLAST2 (Altshul et al., Methods in Enzymology 266:460-480 (1996)). Those comparisons resulting in a BLAST score of 70 (or in some cases 90) or greater that did not encode known proteins were clustered and assembled into a consensus DNA sequence with the program “phrap” (Phil Green, University of Washington, Seattle, Wash.). The consensus sequence obtained therefrom is herein designated DNA105268.

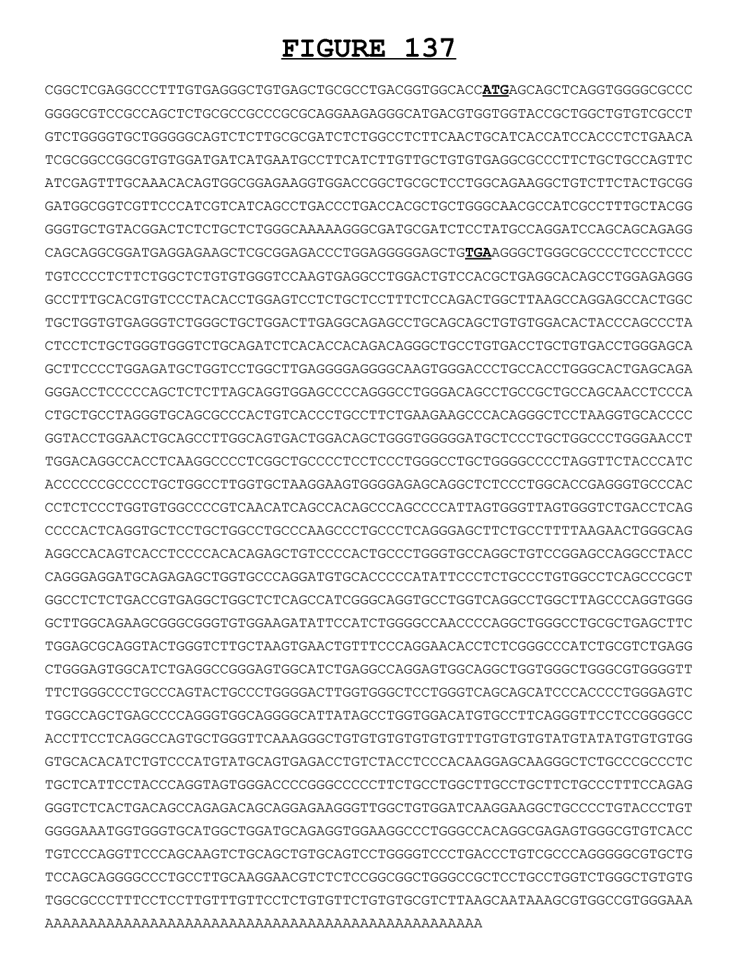

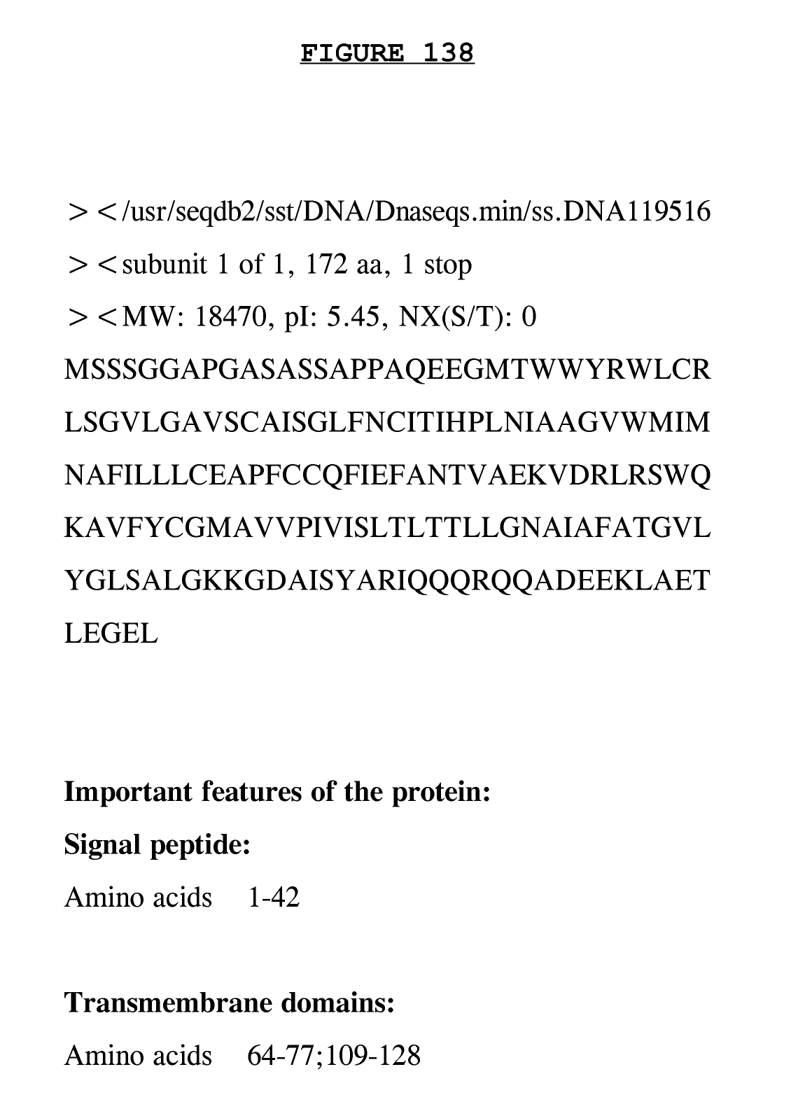

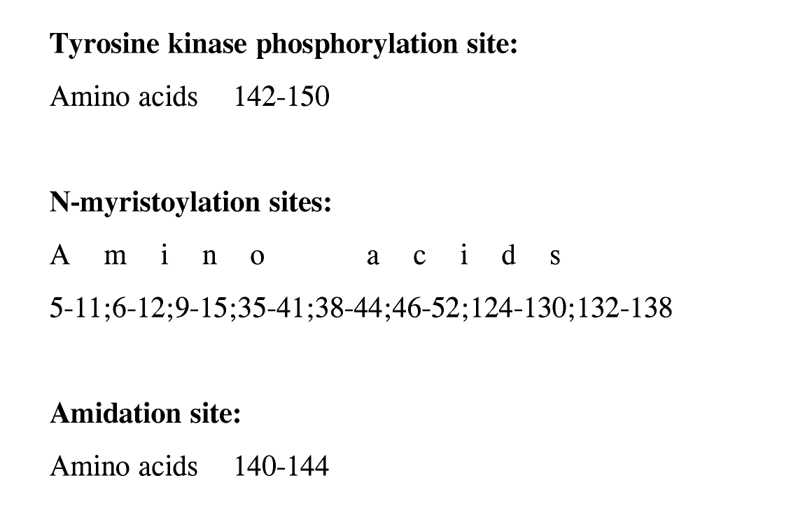

In light of an observed sequence homology between the DNA105268 sequence and an EST sequence encompassed within clone no. 1648912H1 from the LIFESEQ database, clone no. 1648912H1 was purchased and the cDNA insert was obtained and sequenced. It was found herein that that cDNA insert encoded a full-length protein. The sequence of this cDNA insert is shown in FIG. 137 and is herein designated as DNA119596-2797. Clone DNA119596-2797 contains a single open reading frame with an apparent translational initiation site at nucleotide positions 51-53 and ending at the stop codon at nucleotide positions 566-568 (FIG. 137; SEQ ID NO:137). The predicted polypeptide precursor is 172 amino acids long (FIG. 138; SEQ ID NO:138). The full-length PRO9903 protein shown in FIG. 138 has an estimated molecular weight of about 18470 daltons and a pI of about 5.45. Analysis of the full-length PRO9903 sequence shown in FIG. 138 (SEQ ID NO:138) evidences the presence of a variety of important polypeptide domains as shown in FIG. 138, wherein the locations given for those important polypeptide domains are approximate as described above. Clone DNA119596-2797 has been deposited with ATCC on Dec. 22, 1999 and is assigned ATCC deposit no. PTA-1083.

An analysis of the Dayhoff database (version 35.45 SwissProt 35), using the ALIGN-2 sequence alignment analysis of the full-length sequence shown in FIG. 138 (SEQ ID NO:138), evidenced sequence identity between the PRO9903 amino acid sequence and the following Dayhoff sequences: CEF20D1_{—}1, VEU34999_{—}2, POLS_EEVVM, AF075252_{—}2, VEU96408_{—}1, AF004464_{—}1, AF004458_{—}2, AF004472_{—}2, POLS_EEVV3, S63615.

### Example 64

### Isolation of cDNA Clones Encoding Human PRO1106 Polypeptides [UNQ549]

Use of the signal sequence algorithm described in Example 3 above allowed identification of a single Incyte EST sequence. This sequence was then compared to a variety of expressed sequence tag (EST) databases which included public EST databases (e.g., GenBank) and a proprietary EST DNA database (LIFESEQ™, Incyte Pharmaceuticals, Palo Alto, Calif.) to identify existing homologies. The homology search was performed using the computer program BLAST or BLAST2 (Altshul et al., Methods in Enzymology 266:460-480 (1996)). Those comparisons resulting in a BLAST score of 70 (or in some cases 90) or greater that did not encode known proteins were clustered and assembled into a consensus DNA sequence with the program “phrap” (Phil Green, Univ. of Washington, Seattle, Wash.). The consensus sequence obtained therefrom is herein designated DNA56423.

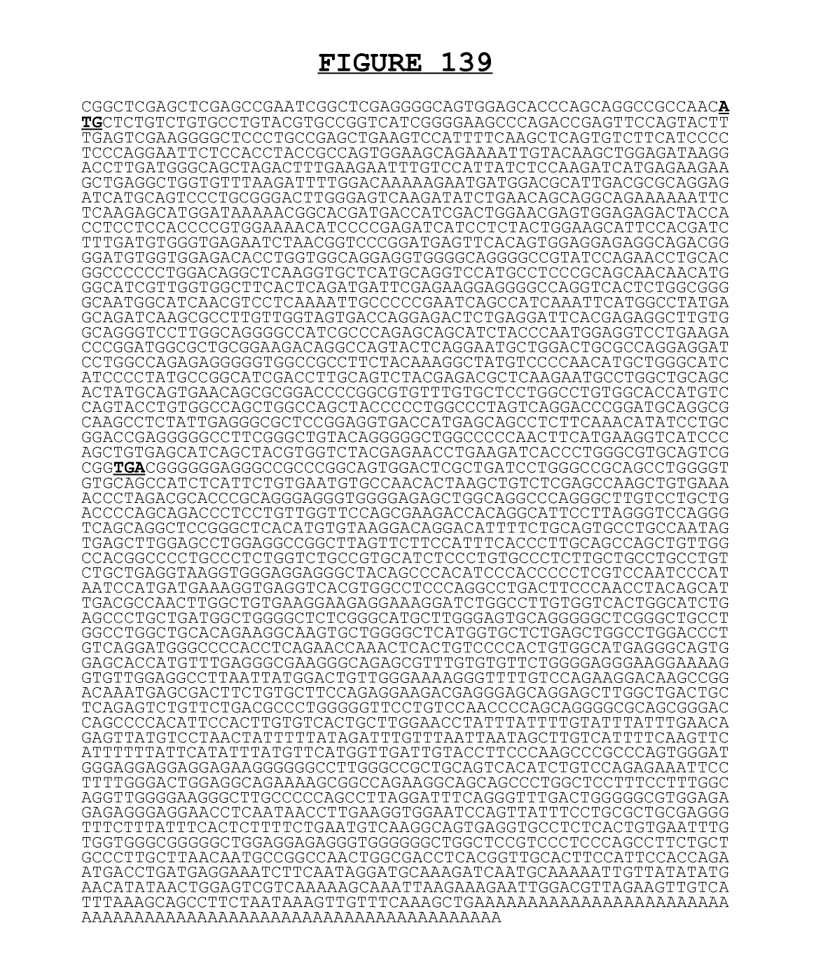

In light of an observed sequence homology between DNA56423 and an EST sequence contained within Incyte EST clone no. 1711247, Incyte EST clone no. 1711247 was obtained and its insert sequenced. It was found that the insert encoded a full-length protein The sequence, designated herein as DNA59609-1470, which is shown in FIG. 139, is the full-length DNA sequence for PRO1106. Clone DNA59609-1470 was deposited with the ATCC on Jun. 9, 1998, and is assigned ATCC deposit no. 209963.

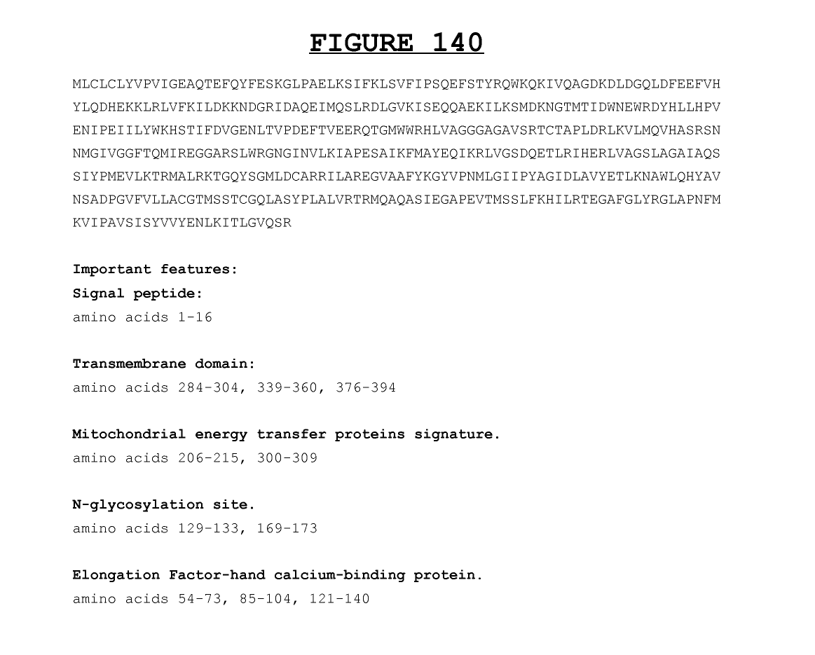

The entire nucleotide sequence of DNA59609-1470 is shown in FIG. 139 (SEQ ID NO:139). Clone DNA59609-1470 contains a single open reading frame with an apparent translational initiation site at nucleotide positions 61-63 and ending at the stop codon at nucleotide positions 1468-1470 of SEQ ID NO:139 (FIG. 139). The predicted polypeptide precursor is 469 amino acids long (FIG. 140; SEQ ID NO:140). The full-length PRO1106 protein shown in FIG. 140 has an estimated molecular weight of about 52,689 daltons and a pI of about 8.68. It is understood that the skilled artisan can construct the polypeptide or nucleic acid encoding therefor to exclude any one or more of all of these domains. For example, the transmembrane domain region(s) and/or either of the amino terminal or carboxyl end can be excluded. Clone DNA59609-1470 has been deposited with ATCC on Jun. 9, 1998 (ATCC No. 209963). It is understood that the deposited clone has the actual nucleic acid sequence and that the sequences provided herein are based on known sequencing techniques.

Analysis of the amino acid sequence of the full-length PRO1106 polypeptide suggests that it possesses significant sequence similarity to the peroxisomal ca-dependent solute carrier, thereby indicating that PRO1106 may be a novel transporter. More specifically, an analysis of the Dayhoff database (version 35.45 SwissProt 35) evidenced sequence identity between the PRO1106 amino acid sequence and at least the following Dayhoff sequences, AF004161_{—}1, IG002N01_{—}25, GDC_BOVIN and BT1_MAIZE.

### Example 65

### Isolation of cDNA Clones Encoding Human PRO1411 Polypeptides [UNQ729]

Use of the signal sequence algorithm described in Example 3 above allowed identification of an EST cluster sequence from an Incyte database. This EST cluster sequence was then compared to a variety of expressed sequence tag (EST) databases which included public EST databases (e.g., GenBank) and a proprietary EST DNA database (LIFESEQ®, Incyte Pharmaceuticals, Palo Alto, Calif.) to identify existing homologies. One or more of the ESTs were derived from a thyroid tissue library. The homology search was performed using the computer program BLAST or BLAST2 (Altshul et al., Methods in Enzymology 266:460-480 (1996)). Those comparisons resulting in a BLAST score of 70 (or in some cases 90) or greater that did not encode known proteins were clustered and assembled into a consensus DNA sequence with the program “phrap” (Phil Green, University of Washington, Seattle, Wash.). The consensus sequence obtained therefrom is herein designated DNA56013.

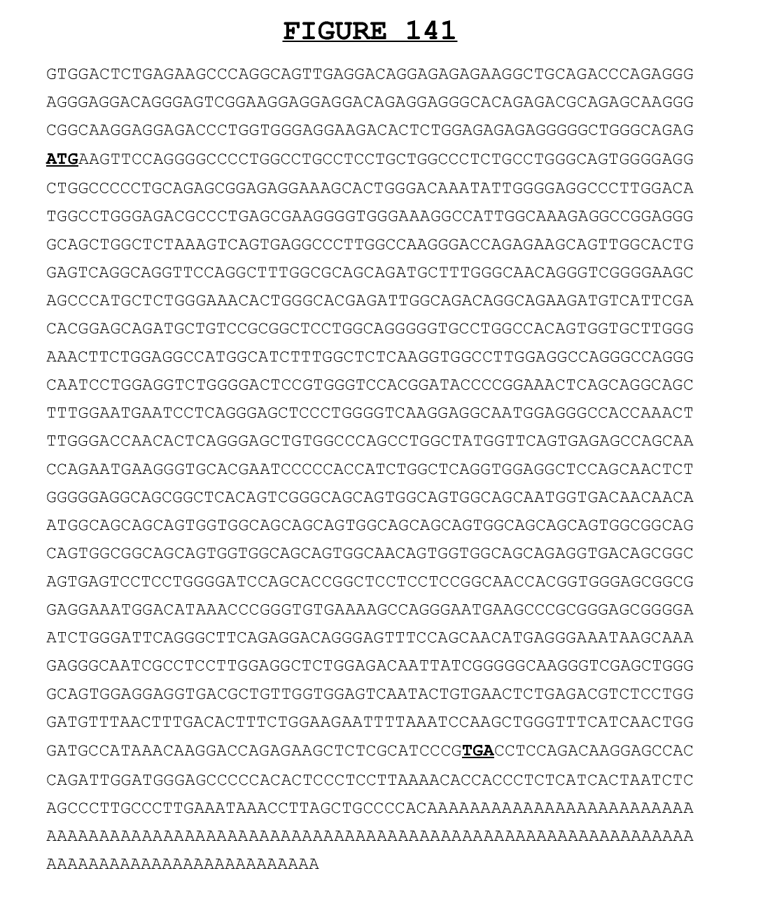

In light of the sequence homology between the DNA56013 sequence and an EST sequence contained within the Incyte EST 1444225, the clone including this EST was purchased and the cDNA insert was obtained and sequenced. The sequence of this cDNA insert is shown in FIG. 141 and is herein designated as DNA59212-1627.

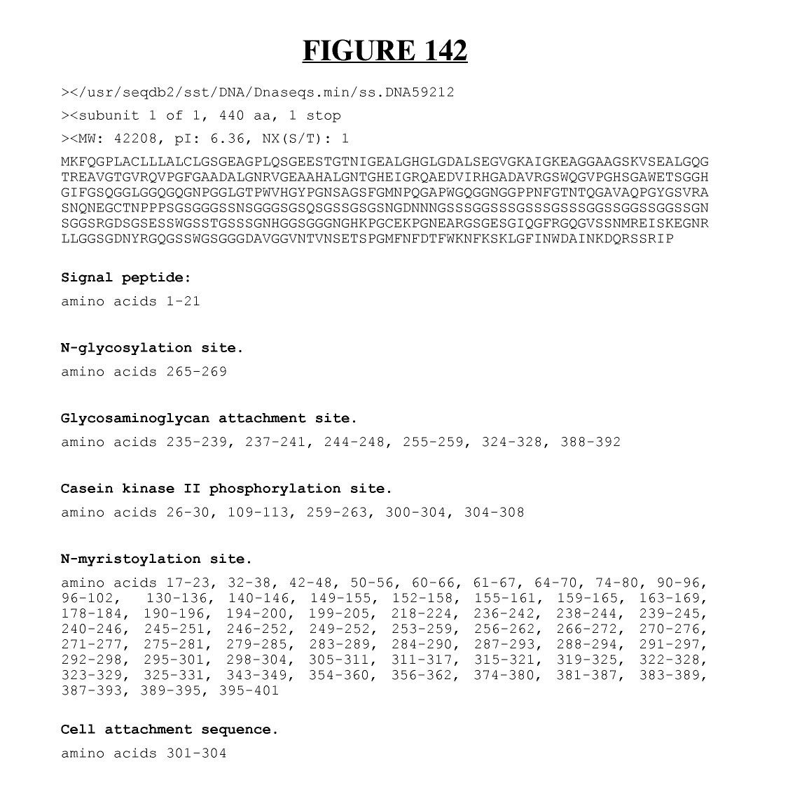

The full length clone shown in FIG. 141 contained a single open reading frame with an apparent translational initiation site at nucleotide positions 184-186 and ending at the stop codon found at nucleotide positions 1504-1506 (FIG. 141; SEQ ID NO:141). The predicted polypeptide precursor (FIG. 142, SEQ ID NO:142) is 440 amino acids long. The signal peptide is at about amino acids 1-21, and the cell attachment site is at about amino acids 301-303 of SEQ ID NO:142. PRO1411 has a calculated molecular weight of approximately 42,208 daltons and an estimated pI of approximately 6.36. Clone DNA59212-1627 was deposited with the ATCC on Sep. 9, 1998 and is assigned ATCC deposit no. 203245.

An analysis of the Dayhoff database (version 35.45 SwissProt 35), using a WU-BLAST2 sequence alignment analysis of the full-length sequence shown in FIG. 142 (SEQ ID NO:142), revealed sequence identity between the PRO1411 amino acid sequence and the following Dayhoff sequences (data from database incorporated herein): MTV023_{—}19, P_R05307, P_W26348, P_P82962, AF000949_{—}1, EBN1_EBV, P_R95107, GRP2_PHAVU, P_R81318, and S74439_{—}1.

### Example 66

### Isolation of cDNA Clones Encoding Human PRO1486 Polypeptides [UNQ755]

A consensus DNA sequence was assembled relative to other EST sequences using phrap as described in Example 1 above. This consensus sequence is designated herein “DNA48897”. Based on the DNA48897 consensus sequence, oligonucleotides were synthesized: 1) to identify by PCR a cDNA library that contained the sequence of interest, and 2) for use as probes to isolate a clone of the full-length coding sequence for PRO1486.

PCR primers (forward and reverse) were synthesized:

| | |
|---|---|
| forward PCR primer | |
| 5′AGGCAGCCACCAGCTCTGTGCTAC3′; | (SEQ ID NO: 258) |
| and | |
| reverse PCR primer | |
| 5′CAGAGAGGGAAGATGAGGAAGCCAGAG3′. | (SEQ ID NO: 259) |

Additionally, a synthetic oligonucleotide hybridization probe was constructed from the consensus DNA48897 sequence which had the following nucleotide sequence:

hybridization probe
(SEQ ID NO: 260)
5′CTGTGCTACTGCCCTTGGACCCTGGGGACCGAGTGTCTCTGC3′.

In order to screen several libraries for a source of a full-length clone, DNA from the libraries was screened by PCR amplification with the PCR primer pair identified above. A positive library was then used to isolate clones encoding the PRO1486 gene using the probe oligonucleotide and one of the PCR primers. RNA for construction of the cDNA libraries was isolated from a human adenocarcinoma cell line.

DNA sequencing of the clones isolated as described above gave the full-length DNA sequence for PRO1486 and the derived protein sequence for PRO1486.

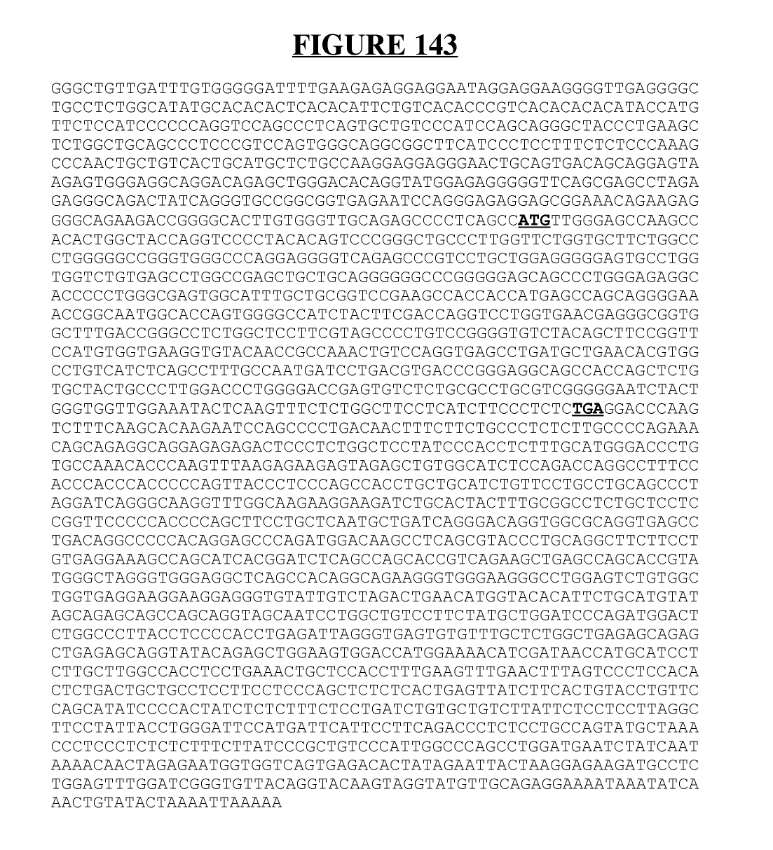

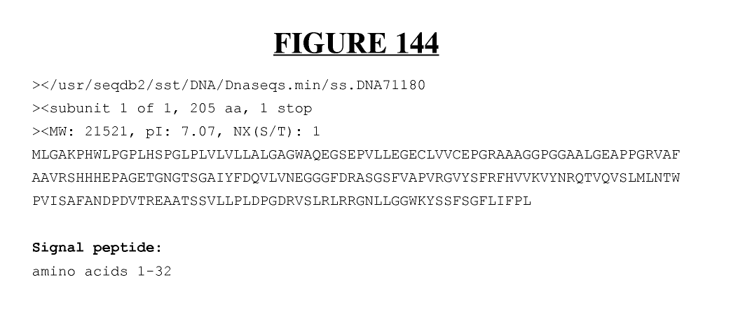

The entire coding sequence of PRO1486 is included in FIG. 143 (SEQ ID NO:143). Clone DNA71180-1655 contains a single open reading frame with an apparent translational initiation site at nucleotide positions 472-474 and an apparent stop codon at nucleotide positions 1087-1089 of SEQ ID NO:143. The predicted polypeptide precursor is 205 amino acids long. The signal peptide is at about amino acids 1-32 of SEQ ID NO:144. Regions similar to those of Cl q and an N-glycosylation site are located as indicated in FIG. 144. Clone DNA71180-1655 has been deposited with the ATCC on Oct. 27, 1998 and is assigned ATCC deposit no. 203403. The full-length PRO1486 protein shown in FIG. 144 has an estimated molecular weight of about 21,521 daltons and a pI of about 7.07.

An analysis of the Dayhoff database (version 35.45 SwissProt 35), using a WU-BLAST2 sequence alignment analysis of the full-length sequence shown in FIG. 144 (SEQ ID NO:144), revealed sequence identity between the PRO1486 amino acid sequence and the following Dayhoff sequences: CERB_HUMAN, CERL_RAT, GEN11893, P_R22263, CA18_HUMAN, C1QC_HUMAN, AF054891_{—}1, A57131, HUMC1Qb2_{—}1, ACR3_MOUSE.

### Example 67

### Isolation of cDNA Clones Encoding Human PRO1565 Polypeptides [UNQ771]

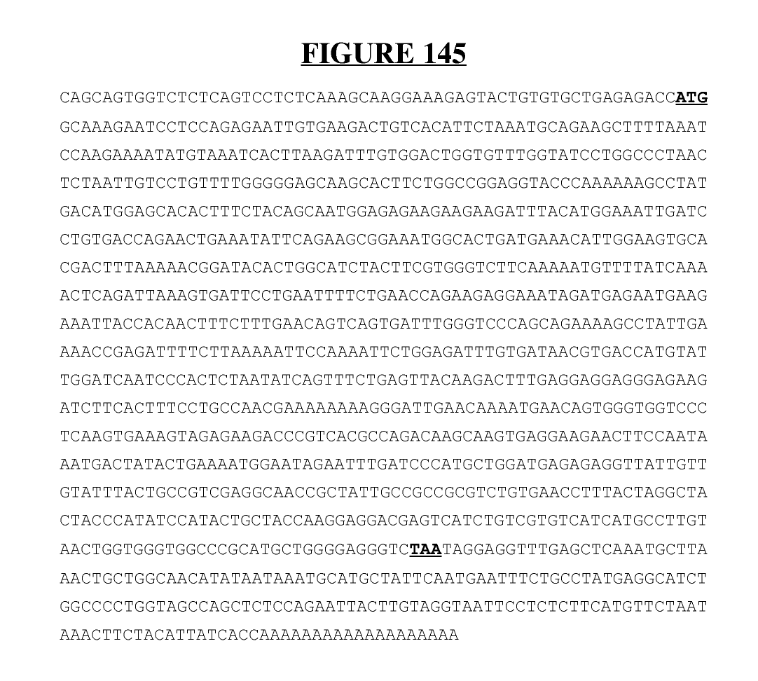

A consensus DNA sequence was assembled relative to other EST sequences using phrap as described in Example 1 above. This consensus sequence is herein designated DNA67183. Based on an observed homology between the DNA67183 consensus sequence and an EST sequence contained within Incyte EST clone no. 2510320, Incyte EST clone no. 2510320 was purchased and its insert was obtained and sequenced. That insert sequence is shown in FIG. 145 and is herein designated DNA73727-1673 (SEQ ID NO:145).

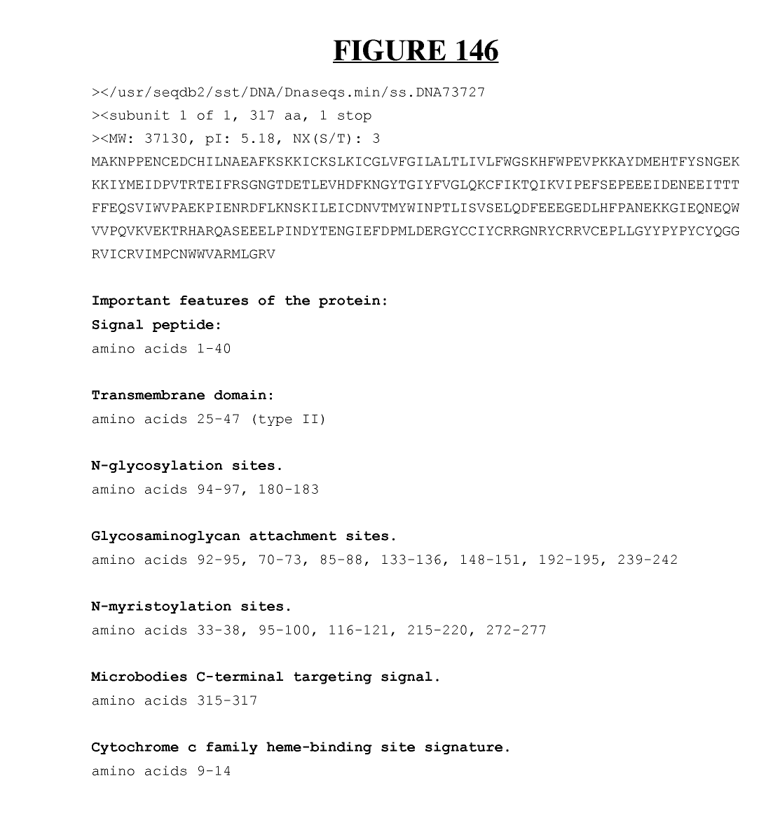

The entire nucleotide sequence of DNA73727-1673 is shown in FIG. 145 (SEQ ID NO:145). Clone DNA73727-1673 contains a single open reading frame with an apparent translational initiation site at nucleotide positions 59-61 and ending at the stop codon at nucleotide positions 1010-1012 (FIG. 145). The predicted polypeptide precursor is 317 amino acids long (FIG. 146; SEQ ID NO:146). The full-length PRO1565 protein shown in FIG. 146 has an estimated molecular weight of about 37,130 daltons and a pI of about 5.18. Analysis of the full-length PRO1565 sequence shown in FIG. 146 (SEQ ID NO:146) evidences the presence of the following: a signal peptide from about amino acid 1 to about amino acid 40, a potential type II transmembrane domain from about amino acid 25 to about amino acid 47, potential N-glycosylation sites from about amino acid 94 to about amino acid 97 and from about amino acid 180 to about amino acid 183, glycosaminoglycan attachment sites from about amino acid 92 to about amino acid 95, from about amino acid 70 to about amino acid 73, from about amino acid 85 to about amino acid 88, from about amino acid 133 to about amino acid 136, from about amino acid 148 to about amino acid 151, from about amino acid 192 to about amino acid 195 and from about amino acid 239 to about amino acid 242, potential N-myristolation sites from about amino acid 33 to about amino acid 38, from about amino acid 95 to about amino acid 100, from about amino acid 116 to about amino acid 121, from about amino acid 215 to about amino acid 220 and from about amino acid 272 to about amino acid 277, a microbodies C-terminal targeting signal sequence from about amino acid 315 to about amino acid 317 and a cytochrome C family heme-binding site signature sequence from about amino acid 9 to about amino acid 14. Clone DNA73727-1673 has been deposited with ATCC on Nov. 3, 1998 and is assigned ATCC deposit no. 203459.

An analysis of the Dayhoff database (version 35.45 SwissProt 35), using a WU-BLAST2 sequence alignment analysis of the full-length sequence shown in FIG. 146 (SEQ ID NO:146), evidenced significant homology between the PRO1565 amino acid sequence and the following Dayhoff sequences: AF051425_{—}1, P_R65490, P_R65488, GRPE_STAAU, RNU31330_{—}1, ACCD_BRANA, D50558_{—}1, HUMAMYAB3_{—}1, P_W34452 and P_P50629.

### Example 68

### Isolation of cDNA clones Encoding Human PRO4399 Polypeptides [UNQ1924]

An Incyte proprietary database was searched and DNA79345 was identified. Based on the DNA79345 sequence, oligonucleotides were synthesized: 1) to identify by PCR a cDNA library that contained the sequence of interest, and 2) for use as probes to isolate a clone of the full-length coding sequence for PRO4399. Forward and reverse PCR primers generally range from 20 to 30 nucleotides and are often designed to give a PCR product of about 100-1000 by in length. The probe sequences are typically 40-55 by in length. In some cases, additional oligonucleotides are synthesized when the consensus sequence is greater than about 1-1.5 kbp. In order to screen several libraries for a full-length clone, DNA from the libraries was screened by PCR amplification, as per Ausubel et al., Current Protocols in Molecular Biology, supra, with the PCR primer pair. A positive library was then used to isolate clones encoding the gene of interest using the probe oligonucleotide and one of the primer pairs.

PCR primers (forward and reverse) were synthesized:

| | |
|---|---|
| forward PCR primer | |
| 5′CGGCAGATTGAAGATGATCGAAAGACAC3′ | (SEQ ID NO: 261) |
| and | |
| reverse PCR primer | |
| 5′GTCTTGTTTCCAAGCTCAGCACTCTTTGG3′. | (SEQ ID NO: 262) |

Additionally, a synthetic oligonucleotide hybridization probe was constructed from the consensus DNA79345 sequence which had the following nucleotide sequence:

hybridization probe
(SEQ ID NO: 263)
5′TCAGGAGTTGAAAGAGAAAATGGACGAGCTCCTGCCTTTGATCCC3′.

In order to screen several libraries for a source of a full-length clone, DNA from the libraries was screened by PCR amplification with the PCR primer pair identified above. A positive library was then used to isolate clones encoding the PRO4399 gene using the probe oligonucleotide and one of the PCR primers.

RNA for construction of the cDNA libraries was isolated from human fetal kidney tissue. The cDNA libraries used to isolate the cDNA clones were constructed by standard methods using commercially available reagents such as those from Invitrogen, San Diego, Calif. The cDNA was primed with oligo dT containing a NotI site, linked with blunt to SalI hemikinased adaptors, cleaved with NolI, sized appropriately by gel electrophoresis, and cloned in a defined orientation into a suitable cloning vector (such as pRKB or pRKD; pRK5B is a precursor of pRK5D that does not contain the SfiI site; see, Holmes et al., Science, 253:1278-1280 (1991)) in the unique XhoI and NotI sites.

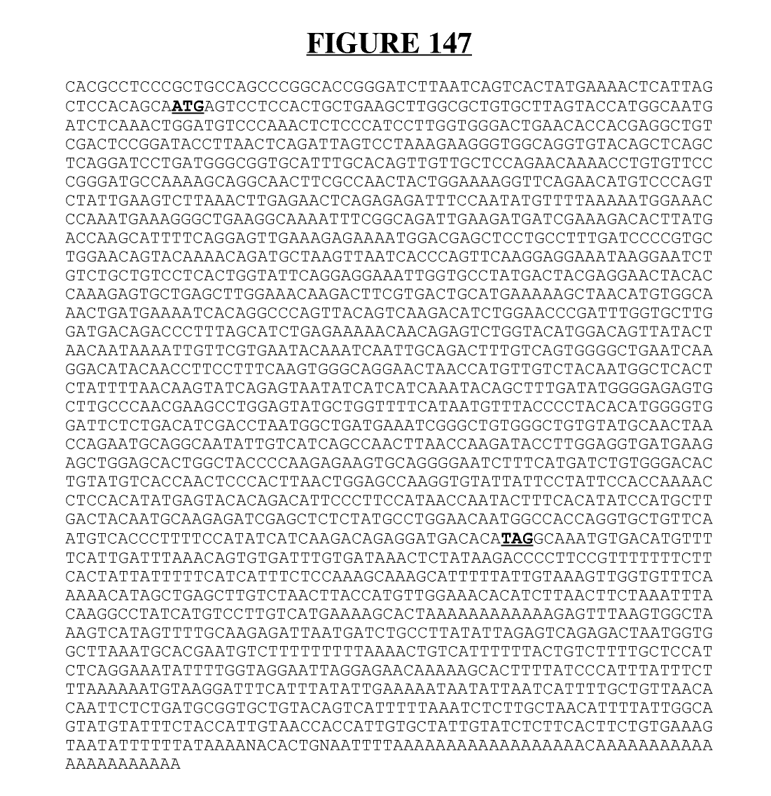

DNA sequencing of the clones isolated as described above gave the full-length DNA sequence for PRO4399 (designated herein as DNA89220-2608 [FIG. 147, SEQ ID NO:147]; and the derived protein sequence for PRO4399.

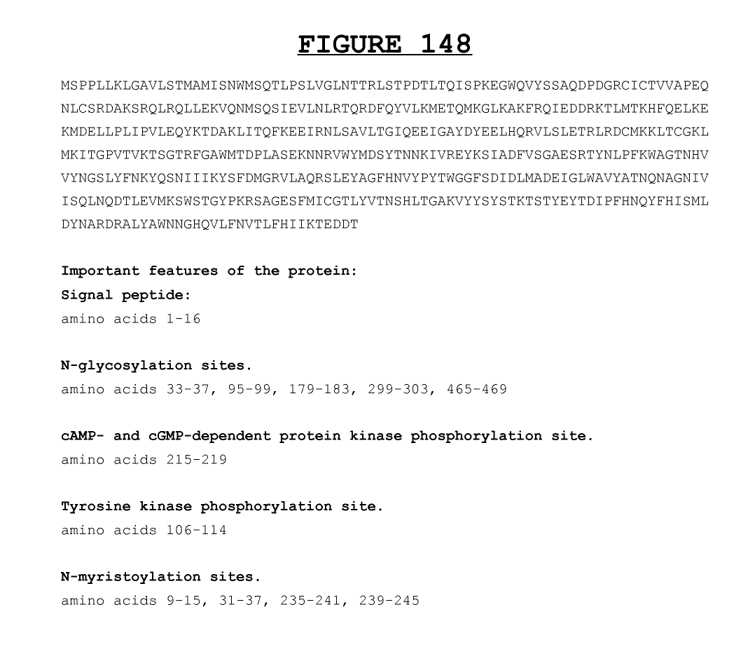

The entire coding sequence of PRO4399 is shown in FIG. 147 (SEQ ID NO:147). Clone DNA89220-2608 contains a single open reading frame with an apparent translational initiation site at nucleotide positions 72-74, and an apparent stop codon at nucleotide positions 1506-1508. The predicted polypeptide precursor is 478 amino acids long. Clone DNA89220-2608 (UNQ1924), designated as DNA89220-2608 has been deposited with ATCC on May 25, 1999 and is assigned ATCC deposit no. PTA-130. The full-length PRO4399 protein shown in FIG. 148 has an estimated molecular weight of about 54930 daltons and a pI of about 8.46.

An analysis of the Dayhoff database (version 35.45 SwissProt 35), using a WU-BLAST2 sequence alignment analysis of the full-length sequence shown in FIG. 148 (SEQ ID NO:148), revealed homology between the PRO4399 amino acid sequence and the following Dayhoff sequences: NOMR_RAT, 173637, D78262_{—}1, I73636, AF028740_{—}1, NOMR_HUMAN, 173635, D78263_{—}1, JE0096, and P_W60670.

### Example 69

### Isolation of cDNA Clones Encoding Human PRO4404 Polypeptides [UNQ1929]

The extracellular domain (ECD) sequences (including the secretion signal sequence, if any) from about 950 known secreted proteins from the Swiss-Prot public database were used to search EST databases. The EST databases included public EST databases (e.g., GenBank), and a proprietary EST database (LIFESEQ®, Incyte Pharmaceuticals, Palo Alto, Calif.). The search was performed using the computer program BLAST or BLAST2 [Altschul et al., Methods in Enzymology, 266:460-480 (1996)] as a comparison of the ECD protein sequences to a 6 frame translation of the EST sequences. Those comparisons resulting in a BLAST score of 70 (or in some cases, 90) or greater that did not encode known proteins were clustered and assembled into consensus DNA sequences with the program “phrap” (Phil Green, University of Washington, Seattle, Wash.).

A consensus DNA sequence encoding PRO4404 was assembled relative to other EST sequences using repeated cycles of phrap. This consensus sequence is designated herein “DNA77609”.

Based on the DNA77609 consensus sequence, oligonucleotides were synthesized: 1) to identify by PCR a cDNA library that contained the sequence of interest, and 2) for use as probes to isolate a clone of the full-length coding sequence for PRO4404. Forward and reverse PCR primers generally range from 20 to 30 nucleotides and are often designed to give a PCR product of about 100-1000 by in length. The probe sequences are typically 40-55 by in length. In some cases, additional oligonucleotides are synthesized when the consensus sequence is greater than about 1-1.5 kbp. In order to screen several libraries for a full-length clone, DNA from the libraries was screened by PCR amplification, as per Ausubel et al., Current Protocols in Molecular Biology, supra, with the PCR primer pair. A positive library was then used to isolate clones encoding the gene of interest using the probe oligonucleotide and one of the primer pairs.

PCR primers (forward and reverse) were synthesized:

| | |
|---|---|
| forward PCR primer | |
| 5′TCAGCAAGGAGACCAACTGCCAGAC3′ | (SEQ ID NO: 264) |
| and | |
| reverse PCR primer | |
| 5′CTGCAGGCAATGTGCATCCATCTG3′. | (SEQ ID NO: 265) |

Additionally, a synthetic oligonucleotide hybridization probe was constructed from the consensus DNA77609 sequence which had the following nucleotide sequence:

hybridization probe
(SEQ ID NO: 266)
5′CCTCAGGGCTACCGCTTCCAGAAGTTAAGCCGAGTGTTGAATCAG3′.

In order to screen several libraries for a source of a full-length clone, DNA from the libraries was screened by PCR amplification with the PCR primer pair identified above. A positive library was then used to isolate clones encoding the PRO4404 gene using the probe oligonucleotide and one of the PCR primers. RNA for construction of the cDNA libraries was isolated from human aortic cells. The cDNA libraries used to isolate the cDNA clones were constructed by standard methods using commercially available reagents such as those from Invitrogen, San Diego, Calif. The cDNA was primed with oligo dT containing a NotI site, linked with blunt to SalI hemikinased adaptors, cleaved with NotI, sized appropriately by gel electrophoresis, and cloned in a defined orientation into a suitable cloning vector (such as pRKB or pRKD; pRK5B is a precursor of pRK5D that does not contain the SfiI site; see, Holmes et al., Science, 253:1278-1280 (1991)) in the unique XhoI and NotI sites.

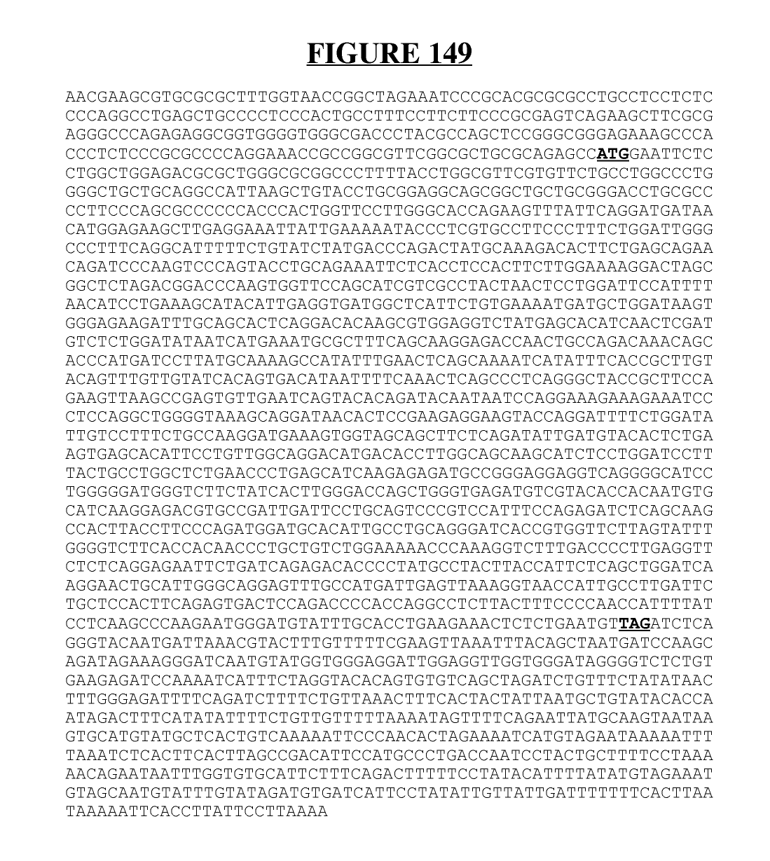

DNA sequencing of the clones isolated as described above gave the full-length DNA sequence for PRO4404 (designated herein as DNA84142-2613 [FIG. 149, SEQ ID NO:149]; and the derived protein sequence for PRO4404.

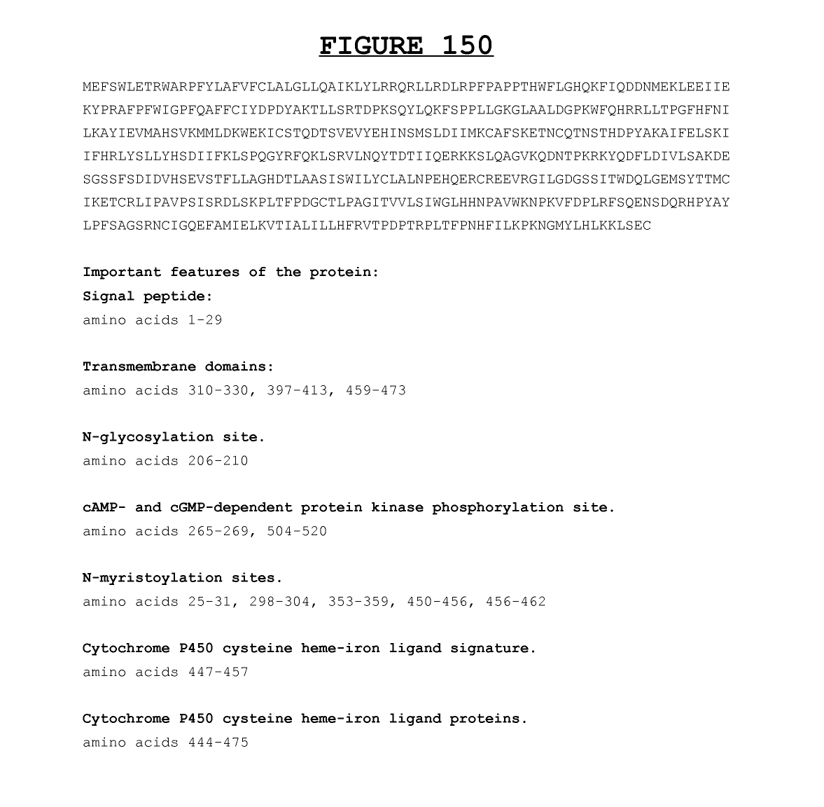

The entire coding sequence of PRO4404 is shown in FIG. 149 (SEQ ID NO:149). Clone DNA84142-2613 contains a single open reading frame with an apparent translational initiation site at nucleotide positions 234-236, and an apparent stop codon at nucleotide positions 1761-1763. The predicted polypeptide precursor is 509 amino acids long. Clone DNA84142-2613 (UNQ1929), designated as DNA84142-2613 has been deposited with ATCC on May 4, 1999 and is assigned ATCC deposit no. PTA-22 The full-length PRO4404 protein shown in FIG. 150 has an estimated molecular weight of about 58875 daltons and a pI of about 8.86.

An analysis of the Dayhoff database (version 35.45 SwissProt 35), using a WU-BLAST2 sequence alignment analysis of the full-length sequence shown in FIG. 150 (SEQ ID NO:150), revealed homology between the PRO4404 amino acid sequence and the following Dayhoff sequences: CP44_RABIT, CP45_RABIT, AB018421_{—}1, CP41_RAT, CP47_RABIT, GEN11564, S47553, AC005336_{—}1, AF054821, AF017002_{—}1.

### Example 70

### Generation and Analysis of Mice Comprising PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 Gene Disruptions

To investigate the role of PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides, disruptions in PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 genes were produced by homologous recombination or retroviral insertion techniques. Specifically, transgenic mice comprising disruptions in PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 genes (i.e., knockout mice) were created by either gene targeting or gene trapping. Mutations were confirmed by southern blot analysis to confirm correct targeting on both the 5′ and 3′ ends. Gene-specific genotyping was also performed by genomic PCR to confirm the loss of the endogenous native transcript as demonstrated by RT-PCR using primers that anneal to exons flanking the site of insertion. Targeting vectors were electroporated into 129 strain ES cells and targeted clones were identified. Targeted clones were microinjected into host blastocysts to produce chimeras. Chimeras were bred with C57 animals to produce F1 heterozygotes. Heterozygotes were intercrossed to produce F2 wildtype, heterozygote and homozygote cohorts which were used for phenotypic analysis. Rarely, if not enough F1 heterozygotes were produced, the F1 hets were bred to wildtype C57 mice to produce sufficient heterozygotes to breed for cohorts to be analyzed for a phenotype. All phenotypic analysis was performed from 12-16 weeks after birth.

### Overall Summary of Phenotypic Results

70.1. Generation and Analysis of Mice Comprising DNA16451-1078 (UNQ153) Gene Disruptions

In these knockout experiments, the gene encoding PRO179 polypeptides (designated as DNA16451-1078) (UNQ153) was disrupted. The gene specific information for these studies is as follows: the mutated mouse gene corresponds to nucleotide reference: BC019491 ACCESSION:BC019491 NID: 18044500 Mus musculus Mus musculus, angiopoietin-like 3, clone MGC:28584 IMAGE:4211688; protein reference: □9R182 ACCESSION:Q9R182NID: Mus musculus (Mouse). ANGIOPOIETIN-RELATED PROTEIN 3 (ANGIOPOIETIN-LIKE 3). MOUSESPTRNRDB; the human gene sequence reference: NM_{—}014495 ACCESSION:NM_{—}014495 NID:7656887 Homo sapiens Homo sapiens angiopoietin-like 3 (ANGPTL3); the human protein sequence corresponds to reference: Q9Y5C1 ACCESSION:Q9Y5C1NID: Homo sapiens (Human). ANGIOPOIETIN-RELATED PROTEIN 3. HUMANSPTRNRDB.

The mouse gene of interest is Angptl3 (angiopoietin-like 3), ortholog of human ANGPTL3. Aliases include hypl, hypolipidemia, ANGPT5, and angiopoietin 5.

ANGPTL3 is a secreted protein expressed primarily in liver that likely binds with receptors on adipocytes or with integrin alpha vbeta 3 on endothelial cells (Conklin et al, Genomics 62(3):477-82 (1999); Shimamura et al, Shimamura et al, Biochem Biophys Res Commun 301(2):604-9 (2003); Camenisch et al, J Biol Chem 277(19):17281-90 (2002)). ANGPTL3 is involved in regulating lipid metabolism (Koishi et al, Nat Genet. 30(2):151-7 (2002)). ANGPTL3 increases plasma free fatty acids by stimulating lipolysis and increases plasma triglycerides by inhibiting adipocyte lipoprotein lipase and, consequently, VLDL clearance (Shimamura et al, Biochem Biophys Res Commun 301(2):604-9 (2003); Shimizugawa et al, J Biol Chem 277(37):33742-8 (2002)). The protein consists of an N-terminal coiled-coil domain, a linker region, and a C-terminal fibrinogen-related domain. Cleavage of ANGPTL3 in the linker region occurs in vivo, generating a fragment containing the coiled-coil domain and a fragment containing the fibrinogen-related domain. The N-terminal coiled-coil domain appears to be most active in increasing plasma triglyceride levels in mice, suggesting that ANGPTL3 is activated by proteolytic cleavage (Ono et al, J Biol Chem 278(43):41804-9 (2003)).

Expression of ANGPTL3 is stimulated by liver X receptor, a nuclear hormone receptor that is activated by cholesterol and synthetic agonist T0901317 (Kaplan et al, J Lipid Res 44(1):136-43 (2003); Inaba et al, J Biol Chem 278(24):21344-51 (2003)), and suppressed by insulin (Inukai et al, Biochem Biophys Res Commun 317(4):1075-9 (2004)). Moreover, ANGPTL3 expression in mice is increased in experimental type I and type II diabetes, suggesting that ANGPTL3 plays an important role in hyperlipidemia associated with diabetes (Inukai et al, Biochem Biophys Res Commun 317(4):1075-9 (2004)).

Koishi and coworkers, Nat Genet. 30(2): 151-7 ((2002) showed that a loss-of-function mutation occurring in the ANGPTL3 gene of mouse strain KK/San causes hypolipidemia. Moreover, they showed that administration of ANGPTL3 protein to these mutant mice increased plasma lipid levels, concluding that ANGPTL3 likely regulates lipid metabolism.

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 18 | 40 | 14 | 72 |
| Expected | 18.0 | 36 | 18.0 | 72 |

Chi-Sq.=1.45 Significance=0.48432454 (hom/n)=0.23 Avg. Litter Size=7
Mutation Type: Homologous Recombination (standard)
Description: Coding exon 1 was targeted (NCBI accession BC019491.1).
1. Wild-type Expression Panel: Expression of the target gene was detected, among 13 adult tissue samples tested by RT-PCR, in kidney; liver; stomach, small intestine, and colon; and heart.
2. QC Expression: Disruption of the target gene was confirmed by Southern hybridization analysis.

70.1.1. Phenotypic Analysis (for Disrupted Gene: DNA16451-1078 (UNQ153)

(a) Overall Phenotypic Summary:

Mutation of the gene encoding the ortholog of human angiopoietin-like 3 (ANGPTL3) resulted in decreased serum cholesterol and triglyceride levels in (−/−) mice. The male and female homozygous mutant mice exhibited notably decreased mean serum cholesterol and triglyceride levels when compared with their gender-matched wild-type littermates and the historical means. Some of the homozygous mutant mice also exhibited hematuria (blood in the urine) and hydronephrosis. In addition, the homozygous mutants exhibited an increased mean serum IL-6 response to LPS challenge. The (−/−) mice also exhibited decreased bone-related measurements. Disruption of the target gene was confirmed by Southern hybridization analysis.

(b) Pathology/CAT Scan

CAT-Scan Protocol:

Mice were injected with a CT contrast agent, Omnipaque 300 (Nycomed Amershan, 300 mg of iodine per ml, 0.25 ml per animal, or 2.50-3.75 g iodine/kg of body weight) intraperitoneally. After resting in the cage for ˜10 minutes, the mouse was then sedated by intraperitoneal injection of Avertin (1.25% 2,2,2,-tribromoethanol, 20 ml/kg body weight). A CAT-scan was performed using a MicroCAT scanner (ImTek, Inc.) with the anesthetized animal lying prone on the test bed. Three dimensional images were reconstructed by the Feldkamp algorithm in a cluster of workstations using an ImTek 3D RECON software.

Results:

Among the 3 (−/−) mice analyzed two male (−/−) mice exhibited moderate hydronephrosis in the right kidney.

(c) Immunology Phenotypic Analysis

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

T lymphocytes (T cells) are an important component of a mammalian immune response. T cells recognize antigens which are associated with a self-molecule encoded by genes within the major histocompatibility complex (MHC). The antigen may be displayed together with MHC molecules on the surface of antigen presenting cells, virus infected cells, cancer cells, grafts, etc. The T cell system eliminates these altered cells which pose a health threat to the host mammal. T cells include helper T cells and cytotoxic T cells. Helper T cells proliferate extensively following recognition of an antigen-MHC complex on an antigen presenting cell. Helper T cells also secrete a variety of cytokines, i.e., lymphokines, which play a central role in the activation of B cells, cytotoxic T cells and a variety of other cells which participate in the immune response.

In many immune responses, inflammatory cells infiltrate the site of injury or infection. The migrating cells may be neutrophilic, eosinophilic, monocytic or lymphocytic as can be determined by histologic examination of the affected tissues. Current Protocols in Immunology, ed. John E. Coligan, 1994, John Wiley & Sons, Inc.

Many immune related diseases are known and have been extensively studied. Such diseases include immune-mediated inflammatory diseases (such as rheumatoid arthritis, immune mediated renal disease, hepatobiliary diseases, inflammatory bowel disease (IBD), psoriasis, and asthma), non-immune-mediated inflammatory diseases, infectious diseases, immunodeficiency diseases, neoplasia, and graft rejection, etc. In the area of immunology, targets were identified for the treatment of inflammation and inflammatory disorders.

In the area of immunology, targets have been identified herein for the treatment of inflammation and inflammatory disorders. Immune related diseases, in one instance, could be treated by suppressing the immune response. Using neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

The following test was performed:

Acute Phase Response:

Test Description: Bacterial lipopolysaccharide (LPS) is an endotoxin, and as such is a potent inducer of an acute phase response and systemic inflammation. The Level I LPS mice were injected intraperitoneally (i.p.) with a sublethal dose of LPS in 200 μL sterile saline using a 26 gauge needle. The doses were based on the average weight of the mice tested at 1 μg/g body weight 3 hours after injection; a 100u1 blood sample was then taken and analyzed for the presence of TNFa, MCP-1, and IL-6 on the FACSCalibur instrument.

Results:

The (−/−) mice exhibited an increased mean serum IL-6 response to LPS challenge when compared with their (+/+) littermates and the historical mean.

In summary, the LPS endotoxin challenge demonstrated that knockout mice deficient in the gene encoding PRO179 polypeptides exhibit immunological abnormalities when compared with their wild-type littermates. In particular, the mutant mice exhibited an increased ability to elicit an immunological response (IL-6 production) when challenged with the LPS endotoxin indicating a proinflammatory response. IL-6 contributes to the later stages of B cell activation. In addition, IL-6 plays a critical role in inducing the acute phase response and systemic inflammation. This suggests that inhibitors or antagonists to PRO179 polypeptides would stimulate the immune system and would find utility in the cases wherein this effect would be beneficial to the individual such as in the case of leukemia, and other types of cancer, and in immunocompromised patients, such as AIDS sufferers. Accordingly, PRO179 polypeptides or agonists thereof would play a role in inhibiting the immune response and would be useful candidates for suppressing harmful immune responses, e.g. in the case of graft rejection or graft-versus-host diseases.

(d) Phenotypic Analysis: Cardiology

In the area of cardiovascular biology, targets were identified herein for the treatment of hypertension, atherosclerosis, heart failure, stroke, various coronary artery diseases, dyslipidemias such as high cholesterol (hypercholesterolemia) and elevated serum triglycerides (hypertriglyceridemia), diabetes and/or obesity. The phenotypic tests included the measurement of serum cholesterol and triglycerides.

Blood Lipids

Procedure: A cohort of 4 wild type, 4 heterozygous and 8 homozygous mice were tested in this assay. High cholesterol levels and increased triglyceride blood levels are recognized risk factors in the development of cardiovascular disease and/or diabetes. Measuring blood lipids facilitates the finding of biological switches that regulate blood lipid levels Inhibition of factors which elevate blood lipid levels may be useful for reducing the risk for cardiovascular disease. In these blood chemistry tests, measurements were recorded using the COBAS Integra 400 (mfr: Roche).

Results:

The male and female (−/−) mice exhibited notably decreased mean serum cholesterol levels (1-2 standard deviations below the historic mean) and decreased mean serum triglyceride levels (>2 standard deviations below the historic means) when compared with their gender-matched (+/+) littermates and the historical means.

As summarized above, the (−/−) mice exhibited notably decreased cholesterol and triglyceride levels when compared with their gender-matched (+/+) littermates and the historical means. Thus, mutant mice deficient in the PRO179 gene can serve as a model for cardiovascular disease Inhibitors or antagonists of PRO179 polypeptides or its encoding gene would be useful in regulating blood lipids such as triglycerides. Thus, antagonists of PRO179 polypeptides would be useful in the treatment of such cardiovascular diseases as hypertension, atherosclerosis, heart failure, stroke, various coronary diseases, hypercholesterolemia, hypertriglyceridemia, diabetes and/or obesity.

Urinalysis

Description

The routine urinalysis is a screening test done to provide a general evaluation of the renal/urinary system. The characteristics for which urine is routinely examined includes tests for protein, glucose, ketones, blood, bilirubin, urobilinogen, nitrate and leukocyte esterase, as well as pH and specific gravity.

Results:

Among the 8 (−/−) mice analyzed, 4 exhibited hematuria (blood in the urine).

(e) Bone Metabolism & Body Diagnostics: Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Bone microCT Analysis:

Procedure: MicroCT was also used to get very sensitive measurements of BMD. One vertebra and 1 femur were taken from a cohort of 4 wild type and 8 homozygous mice. Measurements were taken of lumbar 5 vertebra trabecular bone volume, trabecular thickness, connectivity density and midshaft femur total bone area and cortical thickness. The μCT40 scans provided detailed information on bone mass and architecture. Multiple bones were placed into sample holders and scanned automatically. Instrument software was used to select regions of interest for analysis. Trabecular bone parameters were analyzed in the fifth lumbar vertebrae (LV5) at 16 micrometer resolution and cortical bone parameters were analyzed in the femur midshaft at a resolution of 20 micrometers.

Results:

MicroCT: The (−/−) homozygous mutants exhibited decreased bone-related measurements with decreased trabecular bone volume, number, and connectivity density compared with the (+/+) control littermates.

The (−/−) mice analyzed by bone micro CT analysis exhibited decreased bone measurements when compared with their (+/+) littermates, suggestive of abnormal bone disorders. The (−/−) mice exhibited a negative bone phenotype with abnormal or decreased bone measurements reflective of bone metabolic disorders. The negative bone phenotype indicates that PRO179 polypeptides or agonists thereof would be useful for maintaining bone homeostasis. In addition, PRO179 polypeptides would be useful in bone healing or for the treatment of arthritis or osteoporosis, whereas antagonists (or inhibitors) of PRO179 polypeptides or its encoding gene would lead to abnormal or pathological bone disorders including inflammatory diseases associated with abnormal bone metabolism including arthritis, osteoporosis and osteopenia.

70.2. Generation and Analysis of Mice Comprising DNA23330-1390 (UNQ155) Gene Disruptions

In these knockout experiments, the gene encoding PRO181 polypeptides (designated as DNA23330-1390) (UNQ155) was disrupted. The gene specific information for these studies is as follows: the mutated mouse gene corresponds to nucleotide reference: NM_{—}009919 ACCESSION:NM_{—}009919 NID: gi 27545444 ref NM_{—}009919.1 Mus musculus cornichon homolog (Drosophila) (Cnih); protein reference: 035372 ACCESSION:035372 NID: Mus musculus (Mouse). Cornichon homolog. MOUSE SPTRNRDB; the human gene sequence reference: NM_{—}005776 ACCESSION:NM005776 NID: gi 5031638 ref NM_{—}005776.1 Homo sapiens cornichon homolog (Drosophila) (CNIH); the human protein sequence corresponds to reference: 095406 ACCESSION:095406 NID: Homo sapiens (Human). Cornichon homolog (TGAM77). HUMANSPTRNRDB.

The mouse gene of interest is Cnih (cornichon homolog [Drosophila]), ortholog of human CNIH. Aliases include 0610007J15, CNIL, TGAM77, and cornichon-like.

CNIH is a likely integral plasma membrane protein that is involved in epidermal growth factor signaling during development (Hwang et al, Dev Genes Evol 209(2):120-5 (1999)). Expression of CNIH is upregulated in the early phase of T-cell activation (Utku et al, Biochim Biophys Acta 1449(3):203-10 (1999)). The biochemical function of this protein is not known.

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 15 | 25 | 7 | 47 |
| Expected | 11.75 | 23.5 | 11.75 | 47 |

Chi-Sq.=13.96 Significance=9.303032E-4 (hom/n)=0.16 Avg. Litter Size=7
Mutation Type: Retroviral Insertion (OST)
Description: Retrovial insertion occured in the intron between coding exons 1 and 2 (NCBI accession NM_{—}00919.1).
1. Wild-type Expression Panel: Expression of the target gene was detected in embryonic stem (ES) cells and in all 13 adult tissue samples tested by RT-PCR.
2. QC Expression: RT-PCR analysis revealed that the transcript was absent in the (−/−) mouse analyzed (F-104). Disruption of the target gene was confirmed by Inverse PCR.

70.2.1. Phenotypic Analysis (for Disrupted Gene: DNA23330-1390 (UNQ155)

(a) Overall Phenotypic Summary:

Mutation of the gene encoding the ortholog of human cornichon homolog (Drosophila) (CNIH) resulted in reduced viability of both female and male (−/−) mice. Female and male (−/−) mice also exhibited significant weight loss, decreased total tissue mass and lean body mass, and decreased bone mineral density and bone mineral content. The male homozygous mutant mice also showed decreased mean vertebral trabecular bone volume, thickness, and connectivity density with decreased mean femoral mid-shaft cortical thickness and cross-sectional area. The male (−/−) mutants also exhibited decreased mean serum insulin levels. The female (−/−) mice exhibited an increased mean skin fibroblast proliferation rate. In addition, a decreased anxiety-related response in male (−/−) mice was noted. RT-PCR analysis revealed that the transcript was absent in the homozygous mutant mice.

(b) Phenotypic Analysis: CNS/Neurology

In the area of neurology, analysis focused herein on identifying in vivo validated targets for the treatment of neurological and psychiatric disorders including depression, generalized anxiety disorders, attention deficit hyperactivity disorder, obsessive compulsive disorder, schizophrenia, cognitive disorders, hyperalgesia and sensory disorders. Neurological disorders include the category defined as “anxiety disorders” which include but are not limited to: mild to moderate anxiety, anxiety disorder due to a general medical condition, anxiety disorder not otherwise specified, generalized anxiety disorder, panic attack, panic disorder with agoraphobia, panic disorder without agoraphobia, posttraumatic stress disorder, social phobia, specific phobia, substance-induced anxiety disorder, acute alcohol withdrawal, obsessive compulsive disorder, agoraphobia, bipolar disorder I or II, bipolar disorder not otherwise specified, cyclothymic disorder, depressive disorder, major depressive disorder, mood disorder, substance-induced mood disorder. In addition, anxiety disorders may apply to personality disorders including but not limited to the following types: paranoid, antisocial, avoidant behavior, borderline personality disorders, dependent, histronic, narcissistic, obsessive-compulsive, schizoid, and schizotypal.

Procedure:

Behavioral screens were performed on a cohort of 4 wild type, 4 heterozygous and 8 homozygous mice. All behavioral tests were done between 12 and 16 weeks of age unless reduced viability necessitates earlier testing. These tests included open field to measure anxiety, activity levels and exploration.

Open Field Test:

Several targets of known drugs have exhibited phenotypes in the open field test. These include knockouts of the seratonin transporter, the dopamine transporter (Giros et al., Nature. 1996 Feb. 15; 379(6566):606-12), and the GABA receptor (Homanics et al., Proc Natl Acad Sci USA. 1997 Apr. 15; 94(8):4143-8). An automated open-field assay was customized to address changes related to affective state and exploratory patterns related to learning. First, the field (40×40 cm) was selected to be relatively large for a mouse, thus designed to pick up changes in locomotor activity associated with exploration. In addition, there were 4 holes in the floor to allow for nose-poking, an activity specifically related to exploration. Several factors were also designed to heighten the affective state associated with this test. The open-field test is the first experimental procedure in which the mice are tested, and the measurements that were taken were the subjects' first experience with the chamber. In addition, the open-field was brightly lit. All these factors will heighten the natural anxiety associated with novel and open spaces. The pattern and extent of exploratory activity, and especially the center-to-total distance traveled ratio, may then be able to discern changes related to susceptibility to anxiety or depression. A large arena (40 cm×40 cm, VersaMax animal activity monitoring system from AccuScan Instruments) with infrared beams at three different levels was used to record rearing, hole poke, and locomotor activity. The animal was placed in the center and its activity was measured for 20 minutes. Data from this test was analyzed in five, 4-minute intervals. The total distance traveled (cm), vertical movement number (rearing), number of hole pokes, and the center to total distance ratio were recorded.

The propensity for mice to exhibit normal habituation responses to a novel environment is assessed by determining the overall change in their horizontal locomotor activity across the 5 time intervals. This calculated slope of the change in activity over time is determined using normalized, rather than absolute, total distance traveled. The slope is determined from the regression line through the normalized activity at each of the 5 time intervals. Normal habituation is represented by a negative slope value.

Results:

The male (−/−) mice exhibited an increased median sum time-in-center during open field testing when compared with their gender-matched (+/+) littermates and the historical mean, suggesting a decreased anxiety-like response in the mutants.

A notable difference was observed during open field activity testing. The male (−/−) mice exhibited an increased median sum time in the center area when compared with their gender-matched (+/+) littermates, which is indicative of a decreased anxiety-like response in the mutants. Thus, knockout mice demonstrated a phenotype consistent with depression, generalized anxiety disorders, cognitive disorders, hyperalgesia and sensory disorders and/or bipolar disorders. Thus, PRO181 polypeptides and agonists thereof would be useful for the treatment or amelioration of the symptoms associated with depressive disorders.

(c) Adult Skin Cell Proliferation:

Procedure: Skin cells were isolated from 16 week old animals (2 wild type and 4 homozygous mice). These were developed into primary fibroblast cultures and the fibroblast proliferation rates were measured in a strictly controlled protocol. The ability of this assay to detect hyper-proliferative and hypo-proliferative phenotypes has been demonstrated with p53 and Ku80. Proliferation was measured using Brdu incorporation.

Specifically, in these studies the skin fibroblast proliferation assay was used. An increase in the number of cells in a standardized culture was used as a measure of relative proliferative capacity. Primary fibroblasts were established from skin biopsies taken from wild type and mutant mice. Duplicate or triplicate cultures of 0.05 million cells were plated and allowed to grow for six days. At the end of the culture period, the number of cells present in the culture was determined using a electronic particle counter.

Results:

The female (−/−) mice exhibited an increased mean skin fibroblast proliferation rate when compared with their gender-matched (+/+) littermates.

Thus, homozygous mutant mice demonstrated a hyper-proliferative phenotype. As suggested by these observations, PRO181 polypeptides or agonists thereof could function as tumor suppressors and would be useful in decreasing abnormal cell proliferation.

(d) Bone Metabolism & Body Diagnostics

(1) Tissue Mass & Lean Body Mass Measurements—Dexa

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in total tissue mass (TTM).

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI, i.e., whole body, vertebrae, and both femurs).

Body Measurements (Body Length & Weight):

Body Measurements: A measurement of body length and weight was performed at approximately 16 weeks of age.

Results:

The male and female (−/−) mice exhibited significantly decreased mean body weight when compared with their gender-matched (+/+) littermates and the historical mean. Length data showed no difference between knockout (−/−) mice, heterozygous (+/−) mice and wildtype (+/+) littermate controls.

(2) Bone Metabolism: Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Bone microCT Analysis:

Procedure: MicroCT was also used to get very sensitive measurements of BMD. One vertebra and 1 femur were taken from a cohort of 4 wild type and 8 homozygous mice. Measurements were taken of lumbar 5 vertebra trabecular bone volume, trabecular thickness, connectivity density and midshaft femur total bone area and cortical thickness. The μCT40 scans provided detailed information on bone mass and architecture. Multiple bones were placed into sample holders and scanned automatically. Instrument software was used to select regions of interest for analysis. Trabecular bone parameters were analyzed in the fifth lumbar vertebrae (LV5) at 16 micrometer resolution and cortical bone parameters were analyzed in the femur midshaft at a resolution of 20 micrometers.

Results:

1. DEXA: The male (−/−) mice exhibited decreased mean total tissue mass and lean body mass when compared with their gender-matched (+/+) littermates and the historical means. These mutants also exhibited decreased mean bone mineral content and bone mineral density-related measurements.
2. Micro-CT: The male (−/−) mice exhibited decreased mean vertebral trabecular bone volume, thickness, and connectivity density and decreased mean femoral mid-shaft cortical thickness and cross-sectional area when compared with their gender-matched (+/+) littermates and the historical means.

The (−/−) mice analyzed by DEXA and bone micro CT analysis exhibited decreased bone measurements and decreased body mass measurements when compared with their (+/+) littermates, suggestive of abnormal bone disorders. The (−/−) mice exhibited a negative bone phenotype with abnormal decreased bone measurements reflective of bone metabolic disorders. The negative bone phenotype indicates that PRO181polypeptides or agonists thereof would be useful for maintaining bone homeostasis. In addition, PRO181polypeptides would be useful in bone healing or for the treatment of arthritis or osteoporosis, whereas antagonists (or inhibitors) of PRO181polypeptides or its encoding gene would lead to abnormal or pathological bone disorders including inflammatory diseases associated with abnormal bone metabolism including arthritis, osteoporosis and osteopenia.

(e) Blood Chemistry

Blood chemistry analysis was performed using the COBAS Integra 400 (mfr: Roche) in its clinical settings for running blood chemistry tests on mice.

Insulin Data:

Test Description: Lexicon Genetics uses the Cobra II Series Auto-Gamma Counting System in its clinical settings for running quantitative Insulin assays on mice.

Results:

The male (−/−) mice exhibited a decreased mean serum insulin level when compared with their gender-matched (+/+) littermates and the historical mean.

Summary:

Mutant (−/−) mice deficient in the gene encoding PRO181 polypeptides show a phenotype consistent with tissue wasting diseases marked by decreased total tissue mass and lean body mass. Insulin levels are abnormally low which can be indicative of diabetes. Thus, antagonists or inhibitors of PRO181 polypeptides or its encoding gene would mimic these metabolic related effects. On the other hand, PRO181 polypeptides or agonists thereof would be useful in the prevention and/or treatment of such metabolic disorders as diabetes or other tissue wasting diseases.

(f) Additional Studies

F1 heterozygous animals were intercrossed to generate F2 wild type mice (29 female +/+; 39 male (+/+)), heterozygous mice (65 female +/−; 64 male (+/−)), and homozygous mice (11 female (−/−); 12 male (−/−)) progeny. Half the survival rate was observed for both female and male (−/−) mice. In addition, both female and male UNQ155 knockout mice showed significant weight loss compared to both the wildtype (+/+) and heterozygous (+/−) progeny (measurements taken at clip, wean, six weeks and eight weeks). Thus, UNQ155 knockout mice not only show reduced viability but also significant weight loss. This negative metabolic phenotype indicates that PRO181 polypeptides or agonists thereof are essential for normal growth and development.

70.3. Generation and Analysis of Mice Comprising DNA35668-1171 (UNQ218) Gene Disruptions

In these knockout experiments, the gene encoding PRO244 polypeptides (designated as DNA35668-1171) (UNQ218) was disrupted. The gene specific information for these studies is as follows: the mutated mouse gene corresponds to nucleotide reference: NM_{—}019948 ACCESSION:NM_{—}019948 NID:9910161 Mus musculus Mus musculus C-type (calcium dependent, carbohydrate recognition domain) lectin, superfamily member 9 (Clecsf9); protein reference: □9R0Q8 ACCESSION:Q9R0Q8 NID: Mus musculus (Mouse). MACROPHAGE C-TYPE LECTIN MINCLE (C-TYPE (CALCIUM DEPENDENT, CARBOHYDRATE RECOGNITION DOMAIN) LECTIN, SUPERFAMILY MEMBER 9); the human gene sequence reference: NM_{—}014358 ACCESSION:NM_{—}014358 NID:7657332 Homo sapiens Homo sapiens C-type (calcium dependent, carbohydrate-recognition domain) lectin, superfamily member 9 (CLECSF9); the human protein sequence corresponds to reference: Q9ULY5 ACCESSION:Q9ULY5 NID: Homo sapiens (Human). MACROPHAGE C-TYPE LECTIN MINCLE.

The mouse gene of interest is Clecsf9 (C-type [calcium dependent, carbohydrate recognition domain] lectin, superfamily member 9), ortholog of human CLECSF9. Aliases include MINCLE and macrophage-inducible C-type lectin.

CLECSF9 is a type II plasma membrane protein belonging to the C-type lectin superfamily. The protein consists of a signal anchor and a C-type lectin domain. Proteins with this domain are typically involved in cell adhesion, cell-cell signaling, inflammation, and immune function. Expression of CLECSF9 is induced in macrophages in response to LPS, TNF-alpha, IL-6, and IFN-gamma (Matsumoto et al, J Immunol 163(9):5039-48 (1999); Ebner et al, i Proteins 53(1):44-55 (2003)).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 18 | 30 | 14 | 62 |
| Expected | 15.5 | 31 | 15.5 | 62 |

Chi-Sq.=3.39 Significance=0.18359922 (hom/n)=0.22 Avg. Litter Size=4
Mutation Type: Homologous Recombination (standard)
Description: Coding exons 1 through 3 were targeted (NCBI accession NM_{—}019948.1).
1. Wild-type Expression Panel: Expression of the target gene was detected in spinal cord, eye, thymus, spleen, lung, liver, and adipose among the 13 adult tissue samples tested by RT-PCR.
2. QC Expression: Disruption of the target gene was confirmed by Southern hybridization analysis.

70.3.1. Phenotypic Analysis (for Disrupted Gene: DNA35668-1171 (UNQ218)

(a) Overall Phenotypic Summary:

Mutation of the gene encoding the ortholog of human C-type (calcium dependent, carbohydrate-recognition domain) lectin, superfamily member 9 (CLECSF9) resulted in straub tails during functional observational battery testing in the (−/−) mice. The male (−/−) mice exhibited an increased mean percent total body fat and total fat mass. Disruption of the target gene was confirmed by Southern hybridization analysis.

(b) Phenotypic Analysis: CNS/Neurology

In the area of neurology, analysis focused herein on identifying in vivo validated targets for the treatment of neurological and psychiatric disorders including depression, generalized anxiety disorders, attention deficit hyperactivity disorder, obsessive compulsive disorder, schizophrenia, cognitive disorders, hyperalgesia and sensory disorders. Neurological disorders include the category defined as “anxiety disorders” which include but are not limited to: mild to moderate anxiety, anxiety disorder due to a general medical condition, anxiety disorder not otherwise specified, generalized anxiety disorder, panic attack, panic disorder with agoraphobia, panic disorder without agoraphobia, posttraumatic stress disorder, social phobia, specific phobia, substance-induced anxiety disorder, acute alcohol withdrawal, obsessive compulsive disorder, agoraphobia, bipolar disorder I or II, bipolar disorder not otherwise specified, cyclothymic disorder, depressive disorder, major depressive disorder, mood disorder, substance-induced mood disorder. In addition, anxiety disorders may apply to personality disorders including but not limited to the following types: paranoid, antisocial, avoidant behavior, borderline personality disorders, dependent, histronic, narcissistic, obsessive-compulsive, schizoid, and schizotypal.

Procedure:

Behavioral screens were performed on a cohort of 4 wild type, 4 heterozygous and 8 homozygous mice. All behavioral tests were done between 12 and 16 weeks of age unless reduced viability necessitates earlier testing. These tests included open field to measure anxiety, activity levels and exploration.

Functional Observational Battery (FOB) Test

The FOB is a series of situations applied to the animal to determine gross sensory and motor deficits. A subset of tests from the Irwin neurological screen that evaluates gross neurological function is used. In general, short-duration, tactile, olfactory, and visual stimuli are applied to the animal to determine their ability to detect and respond normally. These simple tests take approximately 10 minutes and the mouse is returned to its home cage at the end of testing.

Results:

Basic Sensory & Motor Observations: Among the 8 (−/−) mice analyzed, 4 exhibited straub tails during functional observational battery testing. These observations are indicative of increased anxiety in the mutant (−/−) mice which is associated with mild to moderate anxiety, anxiety due to a general medical condition, and/or bipolar disorders; hyperactivity; sensory disorders; obsessive-compulsive disorders, schizophrenia or a paranoid personality. Thus, PRO244 polypeptides or agonists thereof can be useful in the treatment of such neurological disorders.

(c) Bone Metabolism & Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Results:

DEXA: The male (−/−) mice exhibited increased mean percent total body fat and total fat mass when compared with their gender-matched (+/+) littermates and the historical means.

These studies suggest that mutant (−/−) non-human transgenic animals exhibit a negative phenotype that would be associated with obesity. Thus, PRO244 polypeptides or agonists thereof are essential for normal growth and metabolic processes and especially would be important in the prevention and/or treatment of obesity.

70.4. Generation and Analysis of Mice Comprising DNA35673-1201 (UNQ221) Gene Disruptions

In these knockout experiments, the gene encoding PRO247 polypeptides (designated as DNA35673-1201) (UNQ221) was disrupted. The gene specific information for these studies is as follows: the mutated mouse gene corresponds to nucleotide reference: BC048152 Mus musculus leucine-rich repeat-containing 8, mRNA (cDNA clone MGC:61242 IMAGE:5708850); protein reference: Q80WG5 ACCESSION:Q80WG5 NID: Mus musculus (Mouse). Leucine-rich repeat-containing protein 8 precursor; the human gene sequence reference: BC051322 Homo sapiens leucine rich repeat containing 8, mRNA (cDNA clone MGC:59975 IMAGE:6250713); the human protein sequence corresponds to reference: Q81WT6 ACCESSION:Q81WT6 NID: Homo sapiens (Human). Leucine-rich repeat-containing protein 8 precursor.

The mouse gene of interest is Lrrc8 (leucine-rich repeat-containing 8), ortholog of human LRRC8. Aliases include MGC49146, MGC61242, mKIAA1437, FLJ10337, and KIAA1437.

LRRC8 is an integral plasma membrane protein that likely functions as a receptor or cell adhesion molecule. The protein consists of four transmembrane segments and an extracellular C-terminal domain of eight leucine-rich repeats. LRRC8 is expressed in T cells and B-lineage cells and is likely to be important for B-cell development. Mutations in the LRRC8 gene can cause agammaglobinuria (Sawada et al, J Clin Invest 112(11):1707-13 (2003); Kubota et al, FEBS Lett 564(1-2):147-52 (2004); Conley, J Clin Invest 112(11):1636-8 (2003); Smits and Kajava, Mol Immunol 41(5):561-2 (2004)).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 19 | 32 | 17 | 68 |
| Expected | 17.0 | 34 | 17.0 | 68 |

Chi-Sq.=0.19 Significance=0.9093729 (hom/n)=0.24 Avg. Litter Size=10

### Mutation Information

Mutation Type: Homologous Recombination (standard)
Description: Coding exon 1 was targeted (NCBI accession BC048152.1).
1. Wild-type Expression Panel: Expression of the target gene was detected in embryonic stem (ES) cells and in all 13 adult tissue samples tested by RT-PCR, except skeletal muscle and bone.
2. QC Expression: Disruption of the target gene was confirmed by Southern hybridization analysis.

70.4.1. Phenotypic Analysis (for Disrupted Gene: DNA35673-1201 (UNQ221)

(a) Overall Phenotypic Summary:

Mutation of the gene encoding the ortholog of human leucine-rich repeat-containing 8 (LRRC8) resulted in an enhanced glucose tolerance. Gene disruption was confirmed by Southern blot.

(b) Phenotypic Analysis: Metabolism-Blood Chemistry/Glucose Tolerance

In the area of metabolism, targets may be identified for the treatment of diabetes. Blood chemistry phenotypic analysis includes blood glucose measurements. The COBAS Integra 400 (mfr: Roche) was used for running blood chemistry tests on the mice. In the area of metabolism, targets may be identified for the treatment of diabetes. Blood chemistry phenotypic analysis includes glucose tolerance tests to measure insulin sensitivity and changes in glucose metabolism. Abnormal glucose tolerance test results may indicate but may not be limited to the following disorders or conditions: Diabetes Type 1 and Type 2, Syndrome X, various cardiovascular diseases and/or obesity.

Procedure: A cohort of 2 wild type and 4 homozygous mice were used in this assay. The glucose tolerance test is the standard for defining impaired glucose homeostasis in mammals. Glucose tolerance tests were performed using a Lifescan glucometer. Animals were injected IP at 2 g/kg with D-glucose delivered as a 20% solution and blood glucose levels were measured at 0, 30, 60 and 90 minutes after injection.

Results:

Glucose Tolerance Test: The male mutant (−/−) mice tested exhibited enhanced glucose tolerance when compared with their gender-matched (+/+) littermates.

In these studies the mutant (−/−) mice showed an increased or enhanced glucose tolerance in the presence of normal fasting glucose at all 3 intervals tested when compared with their gender-matched (+/+) littermates and the historical means. Thus, knockout mice exhibited an increased insulin sensitivity or the opposite phenotypic pattern of an impaired glucose homeostasis, and as such antagonists (inhibitors) to PRO247 polypeptides or its encoding gene would be useful in the treatment of an impaired glucose homeostasis.

70.5. Generation and Analysis of Mice Comprising DNA38260-1180 (UNQ236) Gene Disruptions

In these knockout experiments, the gene encoding PRO269 polypeptides (designated as DNA38260-1180) (UNQ236) was disrupted. The gene specific information for these studies is as follows: the mutated mouse gene corresponds to nucleotide reference: NM_{—}025809 ACCESSION:NM_{—}025809 NID: gi 13385277 ref NM_{—}025809.1 Mus musculus RIKEN cDNA 1200003C23 gene (1200003C23Rik); protein reference: Q9CXA8 ACCESSION:Q9CXA8 NID: Mus musculus (Mouse). 1200003C23RIK PROTEIN; the human gene sequence reference: NM_{—}175060 ACCESSION:NM_{—}175060 NID: gi 28269706 ref NM_{—}175060.1 Homo sapiens chromosome 14 open reading frame 27 (C14orf27); the human protein sequence corresponds to reference: Q86T13 ACCESSION:Q86T13 NID: Homo sapiens (Human). Protein C14orf27 precursor.

The mouse gene of interest is RIKEN cDNA 1200003C23 gene, ortholog of human C14orf27 (chromosome 14 open reading frame 27).

C14orf27 is a hypothetical type I plasma membrane protein, consisting of a signal peptide, a C-type lectin (CTL) domain (SMART accession SM00034), an epidermal growth factor-like domain (SMART accession SM00181), a transmembrane segment, and a cytoplasmic C terminus. The protein is likely to bind with carbohydrate residues on glycoproteins and may function as a ligand, receptor, or cell adhesion molecule.

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 25 | 30 | 12 | 67 |
| Expected | 16.75 | 33.5 | 16.75 | 67 |

Chi-Sq.=6.64 Significance=0.036152836 (hom/n)=0.18 Avg. Litter Size=7

### Mutation Information

Mutation Type: Homologous Recombination (standard)
Description: Coding exon 1 was targeted (NCBI accession (NM_{—}025809.3).
1. Wild-type Expression Panel: Expression of the target gene was detected in all 13 adult tissue samples tested by RT-PCR, except spinal cord, thymus, skeletal muscle, bone, and adipose.
2. QC Expression: Disruption of the target gene was confirmed by Southern hybridization analysis.

70.5.1. Phenotypic Analysis (for Disrupted Gene: DNA38260-1180 (UNQ236)

(a) Overall Phenotypic Summary:

Mutation of the gene encoding the ortholog of human chromosome 14 open reading frame 27 (C14orf27) resulted in decreased bone mineral density measurements in the (−/−) mice. Gene disruption was confirmed by Southern blot.

(b) Expression

UNQ236 (a single transmembrane protein) is expressed in a subset of vessels in murine embryos: specifically in the E9.75 head; E9.75 trunk (fore-gut endoderm; heart; mid-gut endoderm); E10.5 head; and E9.75 anterior trunk (foregut endoderm with liver and pancreatic buds).

GeneLogic expression profile of the UNQ236 human gene shows that the expression is restricted to vascular endothelial cell lines (aortic EC; HMVEC; Pulmonary aortic EC and HUVEC).

(c) Bone Metabolism & Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Bone microCT Analysis:

Procedure: MicroCT was also used to get very sensitive measurements of BMD. One vertebra and 1 femur were taken from a cohort of 4 wild type and 8 homozygous mice. Measurements were taken of lumbar 5 vertebra trabecular bone volume, trabecular thickness, connectivity density and midshaft femur total bone area and cortical thickness. The μCT40 scans provided detailed information on bone mass and architecture. Multiple bones were placed into sample holders and scanned automatically. Instrument software was used to select regions of interest for analysis. Trabecular bone parameters were analyzed in the fifth lumbar vertebrae (LV5) at 16 micrometer resolution and cortical bone parameters were analyzed in the femur midshaft at a resolution of 20 micrometers.

Results:

1. DEXA: The female (−/−) mice exhibited decreased mean bone mineral content, bone mineral content index (BMC/LBM), and bone mineral density in total body, femur, and vertebrae when compared with their gender-matched (+/+) littermates and the historical means.
2. Micro-CT: The male (−/−) mice exhibited decreased mean vertebral trabecular bone volume and connectivity density when compared with their gender-matched (+/+) littermates and the historical means.

The (−/−) mice analyzed by DEXA and bone micro CT analysis exhibited decreased bone measurements when compared with their (+/+) littermates, suggestive of abnormal bone disorders. The (−/−) mice exhibited a negative bone phenotype with abnormal and decreased bone measurements reflective of bone metabolic disorders. The negative bone phenotype indicates that PRO269 polypeptides or agonists thereof would be useful for maintaining bone homeostasis. In addition, PRO269 polypeptides would be useful in bone healing or for the treatment of arthritis or osteoporosis, whereas antagonists (or inhibitors) of PRO269 polypeptides or its encoding gene would lead to abnormal or pathological bone disorders including inflammatory diseases associated with abnormal bone metabolism including arthritis, osteoporosis and osteopenia.

70.6. Generation and Analysis of Mice Comprising DNA37151-1193 (UNQ256) Gene Disruptions

In these knockout experiments, the gene encoding PRO293 polypeptides (designated as DNA37151-1193) (UNQ256) was disrupted. The gene specific information for these studies is as follows: the mutated mouse gene corresponds to nucleotide reference: NM_{—}010732 Mus musculus leucine rich repeat protein 2, neuronal (Lrrn2); protein reference: Q6PHP6 ACCESSION:Q6PHP6 NID: Mus musculus (Mouse). Leucine rich repeat protein 2, neuronal; the human gene sequence reference: NM_{—}006338 ACCESSION:NM_{—}006338 NID: 5453655 Homo sapiens Homo sapiens glioma amplified on chromosome 1 protein (leucine-rich) (GAC1); the human protein sequence corresponds to reference: 075325 ACCESSION:075325 NID: Homo sapiens (Human). GLIOMA AMPLIFIED ON CHROMOSOME 1 PROTEIN PRECURSOR.

The mouse gene of interest is Lrrn2 (leucine rich repeat protein 2, neuronal), ortholog of human LRRN5 (leucine rich repeat neuronal 5). Aliases include NLRR-2, 5730406J09Rik, GAC1, LRANK1, leucine rich and ankyrin repeats 1, glioma amplified on chromosome 1 protein.

LRRN5 is a putative type I plasma membrane expressed primarily in the central nervous system that likely functions as a cell adhesion molecule or receptor. The protein contains a signal peptide, a leucine-rich repeat, a transmembrane segment, and a short, cytoplasmic C terminus. LRRN5 may play a role in development and differentiation of the nervous system. Expression of LRRN5 mRNA is over-expressed in some malignant gliomas (Taguchi et al, Brain Res Mol Brain Res 35(1-2):31-40 (1996); Almeida et al, Oncogene 16(23):2997-3002 (1998); Riemenschneider et al, Int J Cancer 104(6):752-7 (2003)).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 14 | 36 | 17 | 67 |
| Expected | 16.75 | 33.5 | 16.75 | 67 |

Chi-Sq.=4.18 Significance=0.12368715 (hom/n)=0.27 Avg. Litter Size=7
Mutation Type: Homologous Recombination (standard)
Description: Coding exon 1 was targeted (NCBI accession NM_{—}010732.1).
1. Wild-type Expression Panel: Expression of the target gene was detected in embryonic stem (ES) cells and in all 13 adult tissue samples tested by RT-PCR, except lung, skeletal muscle, and bone.
2. QC Expression: Disruption of the target gene was confirmed by Southern hybridization analysis.

70.6.1. Phenotypic Analysis (for Disrupted Gene: DNA37151-1193 (UNQ256)

(a) Overall Phenotypic Summary:

Mutation of the gene encoding the ortholog of human leucine rich repeat neuronal 5 (LRRN5) resulted in neurological abnormalities in (−/−) mice. The homozygous mutant mice exhibited neurological abnormalities when compared with their wild-type littermates, including a decreased anxiety-like response and a decreased sensitivity to pain. Homozygous mutant mice also showed increased total body fat (both percent % and mass (g)). Disruption of the target gene was confirmed by Southern hybridization analysis.

(b) Phenotypic Analysis: CNS/Neurology

In the area of neurology, analysis focused herein on identifying in vivo validated targets for the treatment of neurological and psychiatric disorders including depression, generalized anxiety disorders, attention deficit hyperactivity disorder, obsessive compulsive disorder, schizophrenia, cognitive disorders, hyperalgesia and sensory disorders. Neurological disorders include the category defined as “anxiety disorders” which include but are not limited to: mild to moderate anxiety, anxiety disorder due to a general medical condition, anxiety disorder not otherwise specified, generalized anxiety disorder, panic attack, panic disorder with agoraphobia, panic disorder without agoraphobia, posttraumatic stress disorder, social phobia, specific phobia, substance-induced anxiety disorder, acute alcohol withdrawal, obsessive compulsive disorder, agoraphobia, bipolar disorder I or II, bipolar disorder not otherwise specified, cyclothymic disorder, depressive disorder, major depressive disorder, mood disorder, substance-induced mood disorder. In addition, anxiety disorders may apply to personality disorders including but not limited to the following types: paranoid, antisocial, avoidant behavior, borderline personality disorders, dependent, histronic, narcissistic, obsessive-compulsive, schizoid, and schizotypal.

Procedure:

Behavioral screens were performed on a cohort of 4 wild type, 4 heterozygous and 8 homozygous mice. All behavioral tests were done between 12 and 16 weeks of age unless reduced viability necessitates earlier testing. These tests included open field to measure anxiety, activity levels and exploration.

Functional Observational Battery (FOB) Test—Stress-induced Hyperthermia:

The FOB is a series of situations applied to the animal to determine gross sensory and motor deficits. A subset of tests from the Irwin neurological screen that evaluates gross neurological function is used. In general, short-duration, tactile, olfactory, and visual stimuli are applied to the animal to determine their ability to detect and respond normally. These simple tests take approximately 10 minutes and the mouse is returned to its home cage at the end of testing.

Results:

Anxiety: The male (−/−) mice exhibited a decreased response to stress-induced hyperthermia when compared with their gender-matched (+/+) littermates and the historical mean, suggesting a decreased anxiety-like response in the mutants. Thus, knockout mice demonstrated a phenotype consistent with depression, generalized anxiety disorders, cognitive disorders, hyperalgesia and sensory disorders and/or bipolar disorders. Thus, PRO293 polypeptides and agonists thereof would be useful for the treatment or amelioration of the symptoms associated with depressive disorders.

(c) Bone Metabolism & Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Results:

DEXA: The male (−/−) mice exhibited increased mean percent total body fat and total fat mass when compared with their gender-matched (+/+) littermates and the historical means.

These studies suggest that mutant (−/−) non-human transgenic animals exhibit a negative phenotype that would be associated with obesity. Thus, PRO293 polypeptides or agonists thereof are essential for normal growth and metabolic processes and especially would be important in the prevention and/or treatment of obesity.

70.7. Generation and Analysis of Mice Comprising DNA39975-1210 (UNQ261) Gene Disruptions

In these knockout experiments, the gene encoding PRO298 polypeptides (designated as DNA39975-1210) (UNQ261) was disrupted. The gene specific information for these studies is as follows: the mutated mouse gene corresponds to nucleotide reference: NM_{—}172465 ACCESSION:NM_{—}172465 NID: gi 27369635 ref NM_{—}172465.1 Mus musculus RIKEN cDNA 9530098M12 gene (9530098M12Rik); protein reference: P59268 ACCESSION:P59268 NID: Mus musculus (Mouse). Zinc finger DHHC domain containing protein 9; the human gene sequence reference: NM_{—}016032 Homo sapiens zinc finger, DHHC domain containing 9 (ZDHHC9); the human protein sequence corresponds to reference: □9Y397 ACCESSION:Q9Y397 NID: Homo sapiens (Human). Zinc finger DHHC domain containing protein 9 (Zinc finger protein 379) (CGI-89) (UNQ261/PRO298).

The mouse gene of interest is Zdhhc9 (zinc finger, DHHC domain containing 9), ortholog of human ZDHHC9. Aliases include 6430508G22, 9530098M12Rik, CGI-89, ZNF379, and CGI-89 protein.

ZDHHC9 is a putative membrane protein, consisting of a DHHC zinc finger domain flanked on each side by two transmembrane segments. The function of this protein is not known; however, DHHC zinc finger domains may be involved in protein-protein interactions, protein-DNA interactions, and palmitoyltransferase activity (Pfam accession PF01529).

Targeted or gene trap mutations are generated in strain 129SvEv^{Brd}-derived embryonic stem (ES) cells. The chimeric mice are bred to C57BL/6J albino mice to generate F₁ heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 11 | 13 | 49 | 73 |
| Expected | 18.25 | 36.5 | 18.25 | 73 |

Chi-Sq.=69.82 Significance=6.8988975E-16 (hom/n)=0.67 Avg. Litter Size=8

### Mutation Information

Mutation Type: Homologous Recombination (standard)
Description: Coding exon 1 was targeted (NCBI accession NM_{—}172465.1).
   This project is X-linked.

### Summary of X-linked Gene Distribution by Sex and Genotype

(Only the agouti pups from the male chimeras are included.)

Summary of X-linked Gene Distributions for Sex by Genotype

| | | | |
|---|---|---|---|
| | Agouti F1 | | F1a |
| Progeny | (M chimera × wt) | Progeny | (F het × wt) |

| Sex | wt | het | Sex | wt | het | hemi |
|---|---|---|---|---|---|---|
| M | 10 | 0 | M | 26 | n/a | 35 |
| F | 1 | 18 | F | 33 | 35 | n/a |

1. Wild-type Expression Panel: Expression of the target gene was detected in embryonic stem (ES) cells and in all 13 adult tissue samples tested by RT-PCR, except bone.
2. QC Expression: Disruption of the target gene was confirmed by Southern hybridization analysis.

70.7.1. Phenotypic Analysis (for Disrupted Gene: DNA39975-1210 (UNQ261)

(a) Overall Phenotypic Summary:

Mutation of the gene encoding the ortholog of human zinc finger, DHHC domain containing 9 (ZDHHC9) resulted in increased platelet count in (0/−) mice. This mutation is in an X-linked gene. Both male and female wild-type mice were analyzed, whereas only male hemizygous mutant and female heterozygous mice were analyzed. The male hemizygous (wild-type) and hemizygous mutant mice are designated as (+/+) and (−/−), respectively.

The hemizygous mutant mice (0/−) exhibited an increased mean platelet count as well as decreased serum IgG3 levels when compared with their wild-type littermates and the historical mean. Hot plate testing resulted in a decreased responsiveness in male (−/−) mice. In addition, the knockout mice showed decreased microCT bone measurements. Disruption of the target gene was confirmed by Southern hybridization analysis.

(b) Immunology Phenotypic Analysis

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

T lymphocytes (T cells) are an important component of a mammalian immune response. T cells recognize antigens which are associated with a self-molecule encoded by genes within the major histocompatibility complex (MHC). The antigen may be displayed together with MHC molecules on the surface of antigen presenting cells, virus infected cells, cancer cells, grafts, etc. The T cell system eliminates these altered cells which pose a health threat to the host mammal. T cells include helper T cells and cytotoxic T cells. Helper T cells proliferate extensively following recognition of an antigen-MHC complex on an antigen presenting cell. Helper T cells also secrete a variety of cytokines, i.e., lymphokines, which play a central role in the activation of B cells, cytotoxic T cells and a variety of other cells which participate in the immune response.

In many immune responses, inflammatory cells infiltrate the site of injury or infection. The migrating cells may be neutrophilic, eosinophilic, monocytic or lymphocytic as can be determined by histologic examination of the affected tissues. Current Protocols in Immunology, ed. John E. Coligan, 1994, John Wiley & Sons, Inc.

Many immune related diseases are known and have been extensively studied. Such diseases include immune-mediated inflammatory diseases (such as rheumatoid arthritis, immune mediated renal disease, hepatobiliary diseases, inflammatory bowel disease (IBD), psoriasis, and asthma), non-immune-mediated inflammatory diseases, infectious diseases, immunodeficiency diseases, neoplasia, and graft rejection, etc. In the area of immunology, targets were identified for the treatment of inflammation and inflammatory disorders.

In the area of immunology, targets have been identified herein for the treatment of inflammation and inflammatory disorders. Immune related diseases, in one instance, could be treated by suppressing the immune response. Using neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

The following test was performed:

Hematology Analysis:

Test Description Blood tests are carried out by Abbott's Cell-Dyn 3500R, an automated hematology analyzer. Some of its features include a five-part WBC differential. ‘Patient’ reports can cover over 22 parameters in all.

Results:

Hematology: The (0/−) mice exhibited an increased mean platelet count when compared with their (+/+) littermates and the historical mean.

Thus, mutant mice deficient in the DNA39975-1210 gene resulted in a phenotype related to coagulation disorders. In this regard, inhibitors or antagonists of PRO298 polypeptides would be useful in treating disorders related to abnormal blood coagulation such as hemophilia.

(2) Serum Immunoglobulin Isotyping Assay:

The Serum Immunoglobulin Isotyping Assay is performed using a Cytometric Bead Array (CBA) kit. This assay is used to rapidly identify the heavy and light chain isotypes of a mouse monoclonal antibody in a single sample. The values expressed are “relative fluorescence units” and are based on the detection of kappa light chains. Any value <6 is not significant.

Results:

The (0/−) mice exhibited decreased serum IgG3 compared to their gender-matched littermate controls.

The serum immunoglobulin isotyping assay revealed that hemizygous mutant adults exhibited decreased serum IgG3 levels. Thus, hemizygotes showed an abnormally low serum immunoglobulins compared with the (+/+) littermates. Thus, the gene encoding PRO298 is essential for making immunoglobulins (or gamma globulins). IgG3 immunoglobulins have neutralization effects and to a lesser extent are important for activation of the complement system. These immunological abnormalities suggest that PRO298 polypeptides or agonists thereof would be useful in stimulating the immune system (such as T cell proliferation) and would find utility in the cases wherein this effect would be beneficial to the individual such as in the case of leukemia, and other types of cancer, and in immunocompromised patients, such as AIDS sufferers. Accordingly, inhibitors (antagonists) of PRO298 polypeptides would inhibit the immune response and would be useful candidates for suppressing harmful immune responses, e.g. in the case of graft rejection or graft-versus-host diseases.

(c) Phenotypic Analysis: CNS/Neurology

In the area of neurology, analysis focused herein on identifying in vivo validated targets for the treatment of neurological and psychiatric disorders including depression, generalized anxiety disorders, attention deficit hyperactivity disorder, obsessive compulsive disorder, schizophrenia, cognitive disorders, hyperalgesia and sensory disorders. Neurological disorders include the category defined as “anxiety disorders” which include but are not limited to: mild to moderate anxiety, anxiety disorder due to a general medical condition, anxiety disorder not otherwise specified, generalized anxiety disorder, panic attack, panic disorder with agoraphobia, panic disorder without agoraphobia, posttraumatic stress disorder, social phobia, specific phobia, substance-induced anxiety disorder, acute alcohol withdrawal, obsessive compulsive disorder, agoraphobia, bipolar disorder I or II, bipolar disorder not otherwise specified, cyclothymic disorder, depressive disorder, major depressive disorder, mood disorder, substance-induced mood disorder. In addition, anxiety disorders may apply to personality disorders including but not limited to the following types: paranoid, antisocial, avoidant behavior, borderline personality disorders, dependent, histronic, narcissistic, obsessive-compulsive, schizoid, and schizotypal.

Procedure:

Behavioral screens were performed on a cohort of 4 wild type, 4 heterozygous and 8 homozygous mice. All behavioral tests were done between 12 and 16 weeks of age unless reduced viability necessitates earlier testing.

Hot Plate Testing

Test Description: The hot plate test for nociception is carried out by placing each mouse on a small enclosed 55° C. hot plate. Latency to a hindlimb response (lick, shake, or jump) is recorded, with a maximum time on the hot plate of 30 sec. Each animal is tested once.

Results:

The mutant (−/−) mice exhibited a decreased responsiveness in this testing when compared with their gender-matched (+/+) littermate controls. These results suggest a reduced nociception response.

(d) Bone Metabolism & Body Diagnostics: Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Bone microCT Analysis:

Procedure: MicroCT was also used to get very sensitive measurements of BMD. One vertebra and 1 femur were taken from a cohort of 4 wild type and 8 homozygous mice. Measurements were taken of lumbar 5 vertebra trabecular bone volume, trabecular thickness, connectivity density and midshaft femur total bone area and cortical thickness. The μCT40 scans provided detailed information on bone mass and architecture. Multiple bones were placed into sample holders and scanned automatically. Instrument software was used to select regions of interest for analysis. Trabecular bone parameters were analyzed in the fifth lumbar vertebrae (LV5) at 16 micrometer resolution and cortical bone parameters were analyzed in the femur midshaft at a resolution of 20 micrometers.

Results:

MicroCT: The (−/−) mutants exhibited decreased bone-related measurements with decreased trabecular bone volume, number, and connectivity density compared with the (+/+) control littermates. In addition, the midshaft femur thickness also was decreased in the (−/−) knockout mice.

The (−/−) mice analyzed by bone micro CT analysis exhibited decreased bone measurements when compared with their (+/+) littermates, suggestive of abnormal bone disorders. The (−/−) mice exhibited a negative bone phenotype with abnormal and decreased bone measurements reflective of bone metabolic disorders. The negative bone phenotype indicates that PRO298 polypeptides or agonists thereof would be useful for maintaining bone homeostasis. In addition, PRO298 polypeptides would be useful in bone healing or for the treatment of arthritis or osteoporosis, whereas antagonists (or inhibitors) of PRO298 polypeptides or its encoding gene would lead to abnormal or pathological bone disorders including inflammatory diseases associated with abnormal bone metabolism including arthritis, osteoporosis and osteopenia.

70.8. Generation and Analysis of Mice Comprising DNA43466-1225 (UNQ299) Gene Disruptions

In these knockout experiments, the gene encoding PRO339 polypeptides (designated as DNA43466-1225) (UNQ299) was disrupted. The gene specific information for these studies is as follows: the mutated mouse gene corresponds to nucleotide reference: NM_{—}133913 Mus musculus RIKEN cDNA 2010209012 gene (2010209012Rik); protein reference: Q80TE1 ACCESSION:Q80TE1 NID: Mus musculus (Mouse). MKIAA1402 protein (Fragment); the human gene sequence reference: NM_{—}019015 Homo sapiens chondroitin sulfate glucuronyltransferase (CSGlcA-T); the human protein sequence corresponds to reference: Q6UXD2 ACCESSION:Q6UXD2 NID: Homo sapiens (Human). RLSS299.

The mouse gene of interest is mKIAA1402 protein, ortholog of human CSGlcA-T (chondroitin sulfate glucuronyltransferase). Aliases include KIAA1402.

CSGlcA-T is a type II integral membrane protein that functions as an enzyme, catalyzing the transfer of glucuronic acid to N-acetylgalactosamine on chondroitin sulfate. Like other membrane-associated glycosyltransferases, CSGlcA-T is likely to be located in the Golgi apparatus. CSGlcA-T may play an important role in the elongation of oligosaccharide chains on chondroitin sulfate, a cell surface and extracellular matrix proteoglycan that contributes to cell adhesion, signal transduction, and physical strength of tissues (Gotoh et al, J Biol Chem 277(41):38179-88 (2002)).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 17 | 37 | 16 | 70 |
| Expected | 17.5 | 35 | 17.5 | 70 |

Chi-Sq.=0.84 Significance=0.65704685 (hom/n)=0.24 Avg. Litter Size=8

### Mutation Information

Mutation Type: Homologous Recombination (standard)
Description: Coding exons 1 and 2 were targeted (NCBI accession NM_{—}133913.1).
1. Wild-type Expression Panel: Expression of the target gene was detected in embryonic stem (ES) cells and in all 13 adult tissue samples tested by RT-PCR, except bone.
2. QC Expression: Disruption of the target gene was confirmed by Southern hybridization analysis.

70.8.1. Phenotypic Analysis (for Disrupted Gene: DNA43466-1225 (UNQ299)

(a) Overall Phenotypic Summary:

Mutation of the gene encoding the ortholog of human chondroitin sulfate glucuronyltransferase (CSGlcA-T) resulted in several immunological abnormalities including an elevated LPS response with elevated levels of IL6 and TNF alpha; reduced serum immunoglobulin IgG2a levels, increased B cells in spleen, lymph nodes and Peyer's patches and increased activated/memory T cells. Both heterozygotes and homozygotes exhibited elevated levels of uric acid. Both male and female (−/−) mice exhibited decreased body lengths. Decreased bone-related measurements were also observed in the (−/−) mice. Female knockouts also showed an increased total tissue mass, fat (%) and fat mass (g). Gene disruption was confirmed by Southern blot.

(b) Immunology Phenotypic Analysis

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

T lymphocytes (T cells) are an important component of a mammalian immune response. T cells recognize antigens which are associated with a self-molecule encoded by genes within the major histocompatibility complex

(MHC). The antigen may be displayed together with MHC molecules on the surface of antigen presenting cells, virus infected cells, cancer cells, grafts, etc. The T cell system eliminates these altered cells which pose a health threat to the host mammal. T cells include helper T cells and cytotoxic T cells. Helper T cells proliferate extensively following recognition of an antigen-MHC complex on an antigen presenting cell. Helper T cells also secrete a variety of cytokines, i.e., lymphokines, which play a central role in the activation of B cells, cytotoxic T cells and a variety of other cells which participate in the immune response.

In many immune responses, inflammatory cells infiltrate the site of injury or infection. The migrating cells may be neutrophilic, eosinophilic, monocytic or lymphocytic as can be determined by histologic examination of the affected tissues. Current Protocols in Immunology, ed. John E. Coligan, 1994, John Wiley & Sons, Inc.

Many immune related diseases are known and have been extensively studied. Such diseases include immune-mediated inflammatory diseases (such as rheumatoid arthritis, immune mediated renal disease, hepatobiliary diseases, inflammatory bowel disease (IBD), psoriasis, and asthma), non-immune-mediated inflammatory diseases, infectious diseases, immunodeficiency diseases, neoplasia, and graft rejection, etc. In the area of immunology, targets were identified for the treatment of inflammation and inflammatory disorders.

In the area of immunology, targets have been identified herein for the treatment of inflammation and inflammatory disorders. Immune related diseases, in one instance, could be treated by suppressing the immune response. Using neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

The following test was performed:

(1) Flourescence-activated Cell-sorting (FACS) Analysis

Procedure:

FACS analysis of immune cell composition from peripheral blood was performed including CD4, CD8 and T cell receptor to evaluate T lymphocytes, CD19 for B lymphocytes, CD45 as a leukocyte marker and pan NK for natural killer cells. The FACS analysis was carried out on 2 wild type and 6 homozygous mice and included cells derived from thymus, spleen, bone marrow and lymph node.

In these studies, analyzed cells were isolated from thymus, peripheral blood, spleen, bone marrow and lymph nodes. Flow cytometry was designed to determine the relative proportions of CD4 and CD8 positive T cells, B cells, NK cells and monocytes in the mononuclear cell population. A Becton-Dickinson FACSCalibur 3-laser FACS machine was used to assess immune status. For Phenotypic Assays and Screening, this machine records CD4+/CD8−, CD8+/CD4−, NK, B cell and monocyte numbers in addition to the CD4+/CD8+ ratio. The mononuclear cell profile was derived by staining a single sample of lysed peripheral blood from each mouse with a panel of six lineage-specific antibodies: CD45 PerCP, anti-TCRb APC, CD4 PE, CD8 FITC, pan-NK PE, and CD19 FITC. The two FITC and PE labeled antibodies stain mutually exclusive cell types. The samples were analyzed using a Becton Dickinson FACSCalibur flow cytometer with CellQuest software.

Results:

FACS: The homozygous (−/−) mice exhibited increased percentage of B cells in the spleen, lymph nodes, and Peyer's patches compared to their gender-matched wildtype (+/+) littermates and the historical means. Peyer's patches are aggregates of lymphocytes along the small intestine, especially the ileum. In addition, the (−/−) mice showed increased levels of activated/memory T cells by CD25+ staining and CD62L/CD44 staining. In addition, the (−/−) mice showed decreased levels of NK cells in the spleen compared to their wildtype (+/+) littermates.

In summary, FACS analysis of immune cell composition indicates that knockout (−/−) mice exhibit immunological differences with respect to both B cells and activated T cells Inhibitors or antagonists of PRO339 would be useful in B cell production as well as increasing the numbers of activated/memory T cells, whereas PRO339 polypeptides would be expected to lead to the opposite effects. In addition, the FACS results indicate that the homozygous mutant mice have a decreased mean percentage of natural killer cells. Natural killer cells are the first line of defense to viral infection since these cells have been implicated in viral immunity and in defense against tumors. Natural killer cells or NK cells act as effectors in antibody-dependent cell-mediated cytotoxicity and have been identified by their ability to kill certain lymphoid tumor cell lines in vitro without the need for prior immunization or activation.

(2) Acute Phase Response:

Test Description: Bacterial lipopolysaccharide (LPS) is an endotoxin, and as such is a potent inducer of an acute phase response and systemic inflammation. The Level I LPS mice were injected intraperitoneally (i.p.) with a sublethal dose of LPS in 200 μL sterile saline using a 26 gauge needle. The doses were based on the average weight of the mice tested at 1 μg/g body weight 3 hours after injection; a 100 ul blood sample was then taken and analyzed for the presence of TNFα, MCP-1, and IL-6 on the FACS Calibur instrument.

Results:

The (−/−) mice exhibited an increased mean serum IL6 and TNF-alpha response to LPS challenge when compared with their (+/+) littermates and the historical mean.

In summary, the LPS endotoxin challenge demonstrated that knockout mice deficient in the gene encoding PRO339 polypeptides exhibit immunological abnormalities when compared with their wild-type littermates. In particular, the mutant mice exhibited an increased ability to elicit an immunological response (TNF-alpha and IL-6 production) when challenged with the LPS endotoxin indicating a proinflammatory response. IL-6 and TNF-alpha contribute to the later stages of B cell activation. In addition, IL-6 plays a critical role in inducing the acute phase response and systemic inflammation. This suggests that inhibitors or antagonists to PRO339 polypeptides would stimulate the immune system and would find utility in the cases wherein this effect would be beneficial to the individual such as in the case of leukemia, and other types of cancer, and in immunocompromised patients, such as AIDS sufferers. Accordingly, PRO339 polypeptides or agonists thereof would be useful in inhibiting the immune response and would be useful candidates for suppressing harmful immune responses, e.g. in the case of graft rejection or graft-versus-host diseases.

(3) Serum Immunoglobulin Isotyping Assay:

The Serum Immunoglobulin Isotyping Assay is performed using a Cytometric Bead Array (CBA) kit. This assay is used to rapidly identify the heavy and light chain isotypes of a mouse monoclonal antibody in a single sample. The values expressed are “relative fluorescence units” and are based on the detection of kappa light chains. Any value <6 is not significant.

Results:

The serum immunoglobulin isotyping assay showed decreased or reduced levels of IgG2a in the homozygous (−/−) mice compared to their gender-matched littermate (+/+) controls.

The serum immunoglobulin isotyping assay revealed that homozygous adults exhibited decreased serum IgG2a levels. Thus, homozygotes showed an abnormally low serum immunoglobulins compared with the (+/+) littermates. Thus, the gene encoding PRO339 is essential for making immunoglobulins (or gamma globulins). Likewise, IgG2a immunoglobulins have neutralization effects and to a lesser extent are important for activation of the complement system.

(c) Bone Metabolism & Body Diagnostics

(1) Tissue Mass & Lean Body Mass Measurements—Dexa

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in total tissue mass (TTM).

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI, i.e., whole body, vertebrae, and both femurs).

Body Measurements (Body Length & Weight):

Body Measurements: A measurement of body length and weight was performed at approximately 16 weeks of age.

Results:

Length: The male (−/−) mice exhibited decreased mean body length (1 to 2 standard deviations less) when compared with their gender-matched (+/+) littermates and the historical mean which indicates growth retardation.

(2) Bone Metabolism: Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Bone microCT Analysis:

Procedure: MicroCT was also used to get very sensitive measurements of BMD. One vertebra and 1 femur were taken from a cohort of 4 wild type and 8 homozygous mice. Measurements were taken of lumbar 5 vertebra trabecular bone volume, trabecular thickness, connectivity density and midshaft femur total bone area and cortical thickness. The μCT40 scans provided detailed information on bone mass and architecture. Multiple bones were placed into sample holders and scanned automatically. Instrument software was used to select regions of interest for analysis. Trabecular bone parameters were analyzed in the fifth lumbar vertebrae (LV5) at 16 micrometer resolution and cortical bone parameters were analyzed in the femur midshaft at a resolution of 20 micrometers.

Results:

1. DEXA: The female (−/−) mice exhibited decreased mean bone mineral content, bone mineral content index, and bone mineral density in total body and vertebrae when compared with their gender-matched (+/+) littermates and the historical means. In addition, the female (−/−) mice showed an increased total tissue mass (TTM), fat (%) and fat (g).
2. Micro-CT: The male (−/−) mice exhibited decreased mean vertebral trabecular bone volume, number, and connectivity density, and decreased mean femoral midshaft cortical thickness when compared with their gender-matched (+/+) littermates and the historical means.

Summary:

The (−/−) mice analyzed by DEXA and bone micro CT analysis exhibited decreased bone measurements when compared with their (+/+) littermates, suggestive of abnormal bone disorders. However, female mutant (−/−) mice also exhibited an increased mean percentage of body fat suggestive of an obesity phenotype. These observations suggest that mutant mice deficient in the gene which encodes PRO339 polypeptides leads to metabolic disorders associated with accumulation of fat but also abnormal bone measurements reflective of general metabolic disorders which can be associated with obesity. Thus, PRO339 polypeptides or agonists thereof would be useful in the treatment or prevention of such disorders as obesity or other metabolic diseases. However, the negative bone phenotype would also suggest that PRO339 polypeptides or agonists thereof would be useful for maintaining bone homeostasis. In addition, PRO339 polypeptides would be useful in bone healing or for the treatment of arthritis or osteoporosis, whereas antagonists (or inhibitors) of PRO339 polypeptides or its encoding gene would lead to abnormal or pathological bone disorders including inflammatory diseases associated with abnormal bone metabolism including arthritis, osteoporosis and osteopenia.

(d) Phenotypic Analysis: Metabolism-Blood Chemistry

In the area of metabolism, targets may be identified for the treatment of diabetes. Blood chemistry phenotypic analysis includes blood glucose measurements. The COBAS Integra 400 (mfr: Roche) was used for running blood chemistry tests on the mice. In addition to measuring blood glucose levels the following blood chemistry tests are also routinely performed: Alkaline Phosphatase; Alanine Amino-Transferase; Albumin; Bilirubin; Phosphorous; Creatinine; BUN=Blood Urea Nitrogen; Calcium; Uric Acid; Sodium; Potassium; and Chloride. In the area of metabolism, targets may be identified for the treatment of diabetes.

Results:

Blood chemistry analysis showed elevated uric acids levels (above two standard deviations) in both heterozygous (+/−) mice and homozygous (−/−) mice when compared with their gender-matched (+/+) littermate controls and historical means. Thus, mutant (−/−) and (+/−) mice exhibit a negative phenotype associated with notably elevated uric acid in the blood which is indicative of renal calculi (and associated kidney diseases) which is common in a type of gout (abnormal purine metabolism). PRO339 polypeptides and agonists thereof would be useful in the treatment of such diseases associated with formation of renal calculi and/or abnormal purine metabolism.

70.9. Generation and Analysis of Mice Comprising DNA26288-1239 (UNQ300) Gene Disruptions

In these knockout experiments, the gene encoding PRO341 polypeptides (designated as DNA26288-1239) (UNQ300) was disrupted. The gene specific information for these studies is as follows: the mutated mouse gene corresponds to nucleotide reference: AK006096 Mus musculus adult male testis cDNA, RIKEN full-length enriched library, clone:1700018018 product:hypothetical Myc-type, helix-loop-helix dimerization domain containing protein, full insert sequence; protein reference: Q9DA75 ACCESSION:Q9DA75 NID: Mus musculus (Mouse). 1700018O18Rik protein; the human gene sequence reference: NM_{—}032793 Homo sapiens hypothetical protein FLJ14490 (FLJ14490); the human protein sequence corresponds to reference: Q96F59 ACCESSION:Q96F59 NID: Homo sapiens (Human). Hypothetical protein FLJ90702.

The mouse gene of interest is RIKEN cDNA 1700018O18 gene, ortholog of human hypothetical protein FLJ14490.

Hypothetical protein FLJ14490 is an integral plasma membrane protein, consisting of 10 transmembrane segments. The protein is similar to the melibiose carrier in Escherichia coli (Yazyu et al, J Biol Chem 259(7):4320-6 (1984)), suggesting that the hypothetical human protein functions as a transporter.

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 15 | 46 | 19 | 80 |
| Expected | 20 | 40 | 20 | 80 |

Chi-Sq.=3.09 Significance=0.21331188 (hom/n)=0.22 Avg. Litter Size=8

### Mutation Information

Mutation Type: Homologous Recombination (standard)
Description: Coding exons 1 and 2 were targeted (NCBI accession AK006096.1).
1. Wild-type Expression Panel: Expression of the target gene was detected in embryonic stem (ES) cells and in all 13 adult tissue samples tested by RT-PCR, except skeletal muscle, bone, heart, and adipose.
2. QC Expression: Disruption of the target gene was confirmed by Southern hybridization analysis.

70.9.1. Phenotypic Analysis (for Disrupted Gene: DNA26288-1239 (UNQ300)

(a) Overall Phenotypic Summary:

Mutation of the gene encoding the ortholog of a human hypothetical protein (FLJ14490) resulted in decreased total body fat in (−/−) mice, more notably in males. The (−/−) mice exhibited decreased body weights and lengths and decreased body fat. In addition, female (−/−) mice exhibited myeloid hyperplasia. Reduced levels of NK cell numbers were also observed in the (−/−) mice. The male mutants also exhibited decreased mean serum insulin. Microscopic analysis revealed myeloid hyperplasia in the femoral and sternal bone marrow of the 2 female mutants analyzed. Disruption of the target gene was confirmed by Southern hybridization analysis.

(b) Pathology

Microscopic Observations: Myeloid hyperplasia was observed in the bone marrow of all four female mice examined but was absent in two male mice. Myeloid hyperplasia was present in both sternal and femoral bone marrow. Erythroid hyperplasia in the spleen is a common finding in mice with myeloid hyperplasia in the bone marrow. There was a slight increase in tissue inflammation in the skin, mammary gland, and liver of female mice. Gene Expression: LacZ activity was not detected in the panel of tissues by immunohistochemical analysis.

(c) Immunology Phenotypic Analysis

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

T lymphocytes (T cells) are an important component of a mammalian immune response. T cells recognize antigens which are associated with a self-molecule encoded by genes within the major histocompatibility complex (MHC). The antigen may be displayed together with MHC molecules on the surface of antigen presenting cells, virus infected cells, cancer cells, grafts, etc. The T cell system eliminates these altered cells which pose a health threat to the host mammal. T cells include helper T cells and cytotoxic T cells. Helper T cells proliferate extensively following recognition of an antigen-MHC complex on an antigen presenting cell. Helper T cells also secrete a variety of cytokines, i.e., lymphokines, which play a central role in the activation of B cells, cytotoxic T cells and a variety of other cells which participate in the immune response.

In many immune responses, inflammatory cells infiltrate the site of injury or infection. The migrating cells may be neutrophilic, eosinophilic, monocytic or lymphocytic as can be determined by histologic examination of the affected tissues. Current Protocols in Immunology, ed. John E. Coligan, 1994, John Wiley & Sons, Inc.

Many immune related diseases are known and have been extensively studied. Such diseases include immune-mediated inflammatory diseases (such as rheumatoid arthritis, immune mediated renal disease, hepatobiliary diseases, inflammatory bowel disease (IBD), psoriasis, and asthma), non-immune-mediated inflammatory diseases, infectious diseases, immunodeficiency diseases, neoplasia, and graft rejection, etc. In the area of immunology, targets were identified for the treatment of inflammation and inflammatory disorders.

In the area of immunology, targets have been identified herein for the treatment of inflammation and inflammatory disorders. Immune related diseases, in one instance, could be treated by suppressing the immune response. Using neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

The following test was performed:

Flourescence-activated Cell-sorting (FACS) Analysis

Procedure:

FACS analysis of immune cell composition from peripheral blood was performed including CD4, CD8 and T cell receptor to evaluate T lymphocytes, CD19 for B lymphocytes, CD45 as a leukocyte marker and pan NK for natural killer cells. The FACS analysis was carried out on 2 wild type and 6 homozygous mice and included cells derived from thymus, spleen, bone marrow and lymph node.

In these studies, analyzed cells were isolated from thymus, peripheral blood, spleen, bone marrow and lymph nodes. Flow cytometry was designed to determine the relative proportions of CD4 and CD8 positive T cells, B cells, NK cells and monocytes in the mononuclear cell population. A Becton-Dickinson FACSCalibur 3-laser FACS machine was used to assess immune status. For Phenotypic Assays and Screening, this machine records CD4+/CD8−, CD8+/CD4−, NK, B cell and monocyte numbers in addition to the CD4+/CD8+ ratio. The mononuclear cell profile was derived by staining a single sample of lysed peripheral blood from each mouse with a panel of six lineage-specific antibodies: CD45 PerCP, anti-TCRb APC, CD4 PE, CD8 FITC, pan-NK PE, and CD19 FITC. The two FITC and PE labeled antibodies stain mutually exclusive cell types. The samples were analyzed using a Becton Dickinson FACSCalibur flow cytometer with CellQuest software.

Results:

FACS:

The (−/−) mice exhibited an altered distribution of different leukocyte subsets, characterized by a decreased mean percentage of natural killer cells in the peripheral blood when compared with their wild-type littermates and the historical mean.

In summary, the FACS results indicate that the homozygous mutant mice have an impaired immune system, especially in view of the decreased mean percentage of natural killer cells which is an indicator of a negative phenotype associated with knocking out the DNA26288-1239 gene which encodes PRO341 polypeptides. Natural killer cells are the first line of defense to viral infection since these cells have been implicated in viral immunity and in defense against tumors. Natural killer cells or NK cells act as effectors in antibody-dependent cell-mediated cytotoxicity and have been identified by their ability to kill certain lymphoid tumor cell lines in vitro without the need for prior immunization or activation. However, their known function in host defense is in the early phases of infection with several intracellular pathogens, particularly herpes viruses. Thus, PRO341 polypeptides and agonists thereof would be important for a healthy immune system and would be useful in stimulating the immune system particularly during viral infections.

(d) Bone Metabolism & Body Diagnostics

(1) Tissue Mass & Lean Body Mass Measurements—Dexa

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in total tissue mass (TTM).

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI, i.e., whole body, vertebrae, and both femurs).

Body Measurements (Body Length & Weight):

Body Measurements: A measurement of body length and weight was performed at approximately 16 weeks of age.

Results:

General Observations: The (−/−) mice exhibited a shaky behavior when compared with their (+/+). The (−/−) mice exhibited decreased mean body weight (1-2 standard deviations below the histroical means) and decreased mean body length (>2 standard deviations below the historical means) when compared with their gender-matched (+/+) littermates and the historical means.

(2) Bone Metabolism: Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Results:

1. DEXA: The (−/−) mice exhibited decreased mean percent total body fat and total fat mass when compared with their gender-matched (+/+) littermates and the historical means. The difference was more notable in the males.

The mutant (−/−) mice exhibit depleted total body fat and fat mass suggestive of tissue wasting diseases. Decreased body weight and length measurements in (−/−) mice substantiates a growth retardation phenotype. Thus antagonists (or inhibitors) of PRO341 polypeptides would be expected to mimic this negative phenotype. PRO341 polypeptides or agonists thereof would be useful in maintaining normal fat metabolism and associated growth related metabolism.

(e) Blood Chemistry

Blood chemistry analysis was performed using the COBAS Integra 400 (mfr: Roche) in its clinical settings for running blood chemistry tests on mice.

Insulin Data:

Test Description: Lexicon Genetics uses the Cobra II Series Auto-Gamma Counting System in its clinical settings for running quantitative Insulin assays on mice.

Results:

The male (−/−) mice exhibited a decreased mean serum insulin level when compared with their gender-matched (+/+) littermates and the historical mean.

Mutant (−/−) mice deficient in the gene encoding PRO341 polypeptides show a phenotype consistent with growth retardation, marked by decreased body weight and length and tissue wasting diseases (decreased total body fat (%) and fat mass (g)). Insulin levels are also abnormally low which can be indicative of diabetes. Thus, antagonists or inhibitors of PRO341 polypeptides or its encoding gene would mimic these metabolic and growth related effects. On the other hand, PRO341 polypeptides or agonists thereof would be useful in the prevention and/or treatment of such metabolic disorders as diabetes or other tissue wasting diseases.

70.10. Generation and Analysis of Mice Comprising DNA44176-1244 (UNQ306) Gene Disruptions

In these knockout experiments, the gene encoding PRO347 polypeptides (designated as DNA44176-1244) (UNQ306) was disrupted. The gene specific information for these studies is as follows: the mutated mouse gene corresponds to nucleotide reference: NM_{—}181549 Mus musculus mannose receptor-like precursor (Mrcl); protein reference: Q7TSQ7 ACCESSION:Q7TSQ7 NID: Mus musculus (Mouse). Mannose receptor-like; the human gene sequence reference: NM_{—}182619 Homo sapiens secretory protein LOC348174 (LOC348174); the human protein sequence corresponds to reference: Q8NCF0 ACCESSION:Q8NCF0 NID: Homo sapiens (Human). Hypothetical protein FLJ90292.

The mouse gene of interest is Mrcl (mannose receptor-like precursor), ortholog of human secretory protein LOC348174.

Mrcl is a likely secreted protein, consisting of a signal peptide, an SCP-like extracellular protein domain (Pfam accession PF00188), two epidermal growth factor-like domains, and a C-type lectin domain (Pfam accession PF00059). The function of this protein is not known.

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 20 | 38 | 28 | 86 |
| Expected | 21.5 | 43 | 21.5 | 86 |

Chi-Sq.=0.8 Significance=0.67032003 (hom/n)=0.27 Avg. Litter Size=9

### Mutation Information

Mutation Type: Homologous Recombination (standard)
Description: Coding exons 1 through 3 were targeted (NCBI accession NM_{—}181549.2).
1. Wild-type Expression Panel: Expression of the target gene was detected in embryonic stem (ES) cells and in brain, spinal cord, eye, thymus, kidney, skeletal muscle and heart among the 13 adult tissue samples tested by RT-PCR.
2. QC Expression: Disruption of the target gene was confirmed by Southern hybridization analysis.

70.10.1. Phenotypic Analysis (for Disrupted Gene: DNA44176-1244 (UNQ306)

(a) Overall Phenotypic Summary:

Mutation of the gene encoding the ortholog of a human secretory protein (LOC348174) resulted in increased serum glucose levels and total body fat in (−/−) mice. Both the male and female homozygous mutant mice exhibited increased mean serum glucose levels and increased total body fat when compared with their gender-matched wild-type littermates and the historical means. The (−/−) mice also exhibited decreased bone-related measurements shown by DEXA and microCT measurements. Disruption of the target gene was confirmed by Southern hybridization analysis.

(b) Phenotypic Analysis: Metabolism-Blood Chemistry

In the area of metabolism, targets may be identified for the treatment of diabetes. Blood chemistry phenotypic analysis includes blood glucose measurements. The COBAS Integra 400 (mfr: Roche) was used for running blood chemistry tests on the mice. In the area of metabolism, targets may be identified for the treatment of diabetes.

Results:

Blood Chemistry: The male and female (−/−) mice exhibited increased mean serum glucose levels when compared with their gender-matched (+/+) littermates and the historical means. However, Glucose tolerance testing was normal.

As summarized above, the (−/−) mice exhibited increased mean serum glucose levels suggesting abnormal glucose metabolism or a pre-diabetic condition. In addition, the mutant (−/−) mice also showed increased total body fat and fat mass suggestive of dyslipidemia. Thus, mutant mice deficient in the PRO347 gene can serve as a model for cardiovascular disease associated with elevated levels of fats and blood glucose. PRO347 polypeptides or its encoding gene would be useful in regulating normal blood lipid levels such as triglycerides. Thus, PRO347 polypeptides or agonists thereof would be useful in the treatment of such cardiovascular diseases as hypertension, atherosclerosis, heart failure, stroke, various coronary diseases, hypercholesterolemia, hypertriglyceridemia, diabetes and/or obesity.

(c) Bone Metabolism & Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Bone Micro CT Analysis:

Procedure: MicroCT was also used to get very sensitive measurements of BMD. One vertebra and 1 femur were taken from a cohort of 4 wild type and 8 homozygous mice. Measurements were taken of lumbar 5 vertebra trabecular bone volume, trabecular thickness, connectivity density and midshaft femur total bone area and cortical thickness. The μCT40 scans provided detailed information on bone mass and architecture. Multiple bones were placed into sample holders and scanned automatically. Instrument software was used to select regions of interest for analysis. Trabecular bone parameters were analyzed in the fifth lumbar vertebrae (LV5) at 16 micrometer resolution and cortical bone parameters were analyzed in the femur midshaft at a resolution of 20 micrometers.

Results:

1. DEXA: Both the male and female (−/−) mice exhibited increased mean percent total body fat and total fat mass when compared with their gender-matched (+/+) littermates and the historical means. In addition, male (−/−) mice also exhibited decreased mean bone mineral content and bone mineral content index (BMC/LBM index).
2. Micro-CT: The male (−/−) mice exhibited decreased mean femoral mid-shaft cross-sectional area when compared with their gender-matched (+/+) littermates and the historical mean.

The (−/−) mice analyzed by DEXA and bone micro CT analysis exhibited decreased bone measurements when compared with their (+/+) littermates, suggestive of abnormal bone disorders. However, the mutant (−/−) mice also exhibited an increased mean percentage of body fat suggestive of an obesity phenotype. These observations suggest that mutant mice deficient in the gene which encodes PRO347 polypeptides leads to metabolic disorders associated with accumulation of fat but also abnormal bone measurements reflective of general metabolic disorders which can be associated with obesity. Thus, PRO347 polypeptides or agonists thereof would be useful in the treatment or prevention of such disorders as obesity or other metabolic diseases. However, the negative bone phenotype would also suggest that PRO347 polypeptides or agonists thereof would be useful for maintaining bone homeostasis. In addition, PRO347 polypeptides would be useful in bone healing or for the treatment of arthritis or osteoporosis, whereas antagonists (or inhibitors) of PRO347 polypeptides or its encoding gene would lead to abnormal or pathological bone disorders including inflammatory diseases associated with abnormal bone metabolism including arthritis, osteoporosis and osteopenia.

70.11. Generation and Analysis of Mice Comprising DNA48314-1320 (UNQ332) Gene Disruptions

In these knockout experiments, the gene encoding PRO531 polypeptides (designated as DNA48314-1320) (UNQ332) was disrupted. The gene specific information for these studies is as follows: the mutated mouse gene corresponds to nucleotide reference: NM_{—}053141 Mus musculus protocadherin beta 16 (Pcdhb16); protein reference: Q91Y03 ACCESSION:Q91Y03 NID: Mus musculus (Mouse). Protocadherin beta 16; the human gene sequence reference: NM019120 ACCESSION:NM019120 NID: 14195614 Homo sapiens Homo sapiens protocadherin beta 8 (PCDHB8); the human protein sequence corresponds to reference: Q9UN66 ACCESSION:Q9UN66 NID: Homo sapiens (Human). PROTOCADHERIN BETA 8 PRECURSOR (PCDH-BETA8) (PROTOCADHERIN 31).

The mouse gene of interest is Pcdhb16 (protocadherin beta 16), ortholog of human PCDHB8 (protocadherin beta 8). Aliases include Pcdhb8, PcdhbP, PCDH3I, PCDH-BETA8, and protocadherin-31.

PCDHB8 is a type I plasma membrane protein expressed primarily in neural tissue that functions as a cell adhesion molecule. The protein is likely to play a role in cell-cell interaction (Vanhalst et al, FEBS Lett 495(1-2):120-5 (2001); Yagi and Takeichi, Genes Dev 14(10):1169-80 (2000)).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 13 | 45 | 19 | 77 |
| Expected | 19.25 | 38.5 | 19.25 | 77 |

Chi-Sq.=5.28 Significance=0.07136126 (hom/n)=0.24 Avg. Litter Size=7

### Mutation Information

Mutation Type: Homologous Recombination (standard)
Description: Coding exon 1 was targeted (NCBI accession NM_{—}053 141.2).
1. Wild-type Expression Panel: Expression of the target gene was detected in embryonic stem (ES) cells and in all 13 adult tissue samples tested by RT-PCR, except lung; skeletal muscle; bone; and stomach, small intestine and colon.
2. QC Expression: Disruption of the target gene was confirmed by Southern hybridization analysis.

70.11.1. Phenotypic Analysis (for Disrupted Gene: DNA48314-1320 (UNQ332)

(a) Overall Phenotypic Summary:

Mutation of the gene encoding the ortholog of human protocadherin beta 8 (PCDHB8) resulted in an increase in activated/memory T cells in the spleen of homozygous (−/−) mice. The (−/−) mice also exhibited an increased LBM, BMC and BMC/LBM index. Gene disruption was confirmed by Southern blot.

(b) Bone Metabolism & Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Results:

1. DEXA: The female (−/−) mice exhibited increased mean lean body mass when compared with their gender-matched (+/+) littermates and the historical means. These mutants also exhibited increased bone mineral content and bone mineral density-related measurements, including an increased BMC/LBM index.

In summary, the (−/−) mice exhibited increased mean lean body mass, bone mineral content, and bone mineral density when compared with their gender-matched (+/+) littermates. These results indicate that the knockout mutant phenotype is associated with such bone abnormalities as osteopetrosis. Osteopetrosis is a condition characterized by abnormal thickening and hardening of bone and abnormal fragility of the bones. As such, PRO531 polypeptides or agonists thereof would be beneficial for the treatment of osteopetrosis or other osteo-related diseases. On the other hand, inhibitors or antagonists of PRO531 polypeptides would be useful in bone healing.

(c) Immunology Phenotypic Analysis

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

T lymphocytes (T cells) are an important component of a mammalian immune response. T cells recognize antigens which are associated with a self-molecule encoded by genes within the major histocompatibility complex (MHC). The antigen may be displayed together with MHC molecules on the surface of antigen presenting cells, virus infected cells, cancer cells, grafts, etc. The T cell system eliminates these altered cells which pose a health threat to the host mammal. T cells include helper T cells and cytotoxic T cells. Helper T cells proliferate extensively following recognition of an antigen-MHC complex on an antigen presenting cell. Helper T cells also secrete a variety of cytokines, i.e., lymphokines, which play a central role in the activation of B cells, cytotoxic T cells and a variety of other cells which participate in the immune response.

In many immune responses, inflammatory cells infiltrate the site of injury or infection. The migrating cells may be neutrophilic, eosinophilic, monocytic or lymphocytic as can be determined by histologic examination of the affected tissues. Current Protocols in Immunology, ed. John E. Coligan, 1994, John Wiley & Sons, Inc.

Many immune related diseases are known and have been extensively studied. Such diseases include immune-mediated inflammatory diseases (such as rheumatoid arthritis, immune mediated renal disease, hepatobiliary diseases, inflammatory bowel disease (IBD), psoriasis, and asthma), non-immune-mediated inflammatory diseases, infectious diseases, immunodeficiency diseases, neoplasia, and graft rejection, etc. In the area of immunology, targets were identified for the treatment of inflammation and inflammatory disorders.

In the area of immunology, targets have been identified herein for the treatment of inflammation and inflammatory disorders. Immune related diseases, in one instance, could be treated by suppressing the immune response. Using neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

The following test was performed:

Flourescence-activated Cell-sorting (FACS) Analysis

Procedure:

FACS analysis of immune cell composition from peripheral blood was performed including CD4, CD8 and T cell receptor to evaluate T lymphocytes, CD19 for B lymphocytes, CD45 as a leukocyte marker and pan NK for natural killer cells. The FACS analysis was carried out on 2 wild type and 6 homozygous mice and included cells derived from thymus, spleen, bone marrow and lymph node.

In these studies, analyzed cells were isolated from thymus, peripheral blood, spleen, bone marrow and lymph nodes. Flow cytometry was designed to determine the relative proportions of CD4 and CD8 positive T cells, B cells, NK cells and monocytes in the mononuclear cell population. A Becton-Dickinson FACSCalibur 3-laser FACS machine was used to assess immune status. For Phenotypic Assays and Screening, this machine records CD4+/CD8−, CD8+/CD4−, NK, B cell and monocyte numbers in addition to the CD4+/CD8+ratio. The mononuclear cell profile was derived by staining a single sample of lysed peripheral blood from each mouse with a panel of six lineage-specific antibodies: CD45 PerCP, anti-TCRb APC, CD4 PE, CD8 FITC, pan-NK PE, and CD19 FITC. The two FITC and PE labeled antibodies stain mutually exclusive cell types. The samples were analyzed using a Becton Dickinson FACSCalibur flow cytometer with CellQuest software.

Results:

FACS: The mutant (−/−) mice exhibited an increase in activated/memory T cells in the spleen compared to their gender-matched (+/+) littermates and the historical means.

In summary, FACS analysis of immune cell composition indicates that knockout mice (−/−) exhibit immunological differences with respect to activated/memory T cells. From these observations, PRO531 polypeptides or the gene encoding PRO531 appear to act as a negative regulator of T cell proliferation. Thus, PRO531 polypeptides or agonists thereof would be beneficial as a negative regulator of T cell proliferation in those instances wherein a pronounced T-cell proliferation is present such as occurs in rheumatoid arthritis patients. Inhibitors or antagonists of PRO531 would be useful in increasing the numbers of activated/memory T cells, whereas PRO531 polypeptides or agonists thereof would be expected to lead to the opposite effects.

70.12. Generation and Analysis of Mice Comprising DNA49141-1431 (UNQ338) Gene Disruptions

In these knockout experiments, the gene encoding PRO537 polypeptides (designated as DNA49141-1431) (UNQ338) was disrupted. The gene specific information for these studies is as follows: the mutated mouse gene corresponds to nucleotide reference: AK014425 Mus musculus 18 days pregnant adult female placenta and extra embryonic tissue cDNA, RIKEN full-length enriched library, clone:3830408D24 product:hypothetical Histidine-rich region containing protein, full insert sequence; protein reference: Q9D6B9 ACCESSION: Q9D6B9 NID: Mus musculus (Mouse). 3830408D24Rik protein; the human gene sequence reference: AY358408 Homo sapiens clone DNA49141 LGLL338 (UNQ338); the human protein sequence corresponds to reference: AAQ88774 LGLL338 [Homo sapiens].

The mouse gene of interest is RIKEN cDNA 3830408D24 gene, ortholog of human UNQ338 (Homo sapiens clone DNA49141 LGLL338 [UNQ338] mRNA).

UNQ338 is a putative secreted or type II plasma membrane protein. The 115-amino acid protein contains a signal peptide or signal anchor and a weakly predicted glycine rich protein (GRP) domain. This domain is found in plant proteins that are induced in response to stress (Pfam accession PF07172).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 24 | 43 | 18 | 85 |
| Expected | 21.25 | 42.5 | 21.25 | 85 |

Chi-Sq.=2.61 Significance=0.27117255 (hom/n)=0.21 Avg. Litter Size=8

### Mutation Information

Mutation Type: Homologous Recombination (standard)
Description: Coding exon 1 was targeted (NCBI accession AK014425).
1. Wild-type Expression Panel: Expression of the target gene was detected in embryonic stem (ES) cells and in all 13 adult tissue samples tested by RT-PCR.
2. QC Expression: Disruption of the target gene was confirmed by Southern hybridization analysis.

70.12.1. Phenotypic Analysis (for Disrupted Gene: DNA49141-1431 (UNQ338)

(a) Overall Phenotypic Summary:

Mutation of the gene encoding the ortholog of human Homo sapiens clone DNA49141 LGLL338 (UNQ338) resulted in immunological abnormalities and impaired gastrointestinal mobility in (−/−) mice. The mutant (−/−) mice showed numerous immunological abnormalities with decreased mono cyte counts, increased mean percentage of B cells and decreased percentages of CD4 and CD8 cells. The mutant (−/−) mice also exhibited increased bone-related measurements. Disruption of the target gene was confirmed by Southern hybridization analysis.

(b) Immunology Phenotypic Analysis

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

T lymphocytes (T cells) are an important component of a mammalian immune response. T cells recognize antigens which are associated with a self-molecule encoded by genes within the major histocompatibility complex (MHC). The antigen may be displayed together with MHC molecules on the surface of antigen presenting cells, virus infected cells, cancer cells, grafts, etc. The T cell system eliminates these altered cells which pose a health threat to the host mammal. T cells include helper T cells and cytotoxic T cells. Helper T cells proliferate extensively following recognition of an antigen-MHC complex on an antigen presenting cell. Helper T cells also secrete a variety of cytokines, i.e., lymphokines, which play a central role in the activation of B cells, cytotoxic T cells and a variety of other cells which participate in the immune response.

In many immune responses, inflammatory cells infiltrate the site of injury or infection. The migrating cells may be neutrophilic, eosinophilic, monocytic or lymphocytic as can be determined by histologic examination of the affected tissues. Current Protocols in Immunology, ed. John E. Coligan, 1994, John Wiley & Sons, Inc.

Many immune related diseases are known and have been extensively studied. Such diseases include immune-mediated inflammatory diseases (such as rheumatoid arthritis, immune mediated renal disease, hepatobiliary diseases, inflammatory bowel disease (IBD), psoriasis, and asthma), non-immune-mediated inflammatory diseases, infectious diseases, immunodeficiency diseases, neoplasia, and graft rejection, etc. In the area of immunology, targets were identified for the treatment of inflammation and inflammatory disorders.

In the area of immunology, targets have been identified herein for the treatment of inflammation and inflammatory disorders. Immune related diseases, in one instance, could be treated by suppressing the immune response. Using neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

The following test was performed:

Hematology Analysis:

Test Description Blood tests are carried out by Abbott's Cell-Dyn 3500R, an automated hematology analyzer. Some of its features include a five-part WBC differential. ‘Patient’ reports can cover over 22 parameters in all.

Results:

Hematology: The (−/−) mice exhibited a decreased mean absolute monocyte count when compared with their (+/+) littermates and the historical mean.

In summary, the hematology results indicate that the homozygous mutant mice exhibited an decreased monocyte count compared to their littermate controls indicating depressed levels of precursors of macrophages. These results indicate that the homozygous (−/−) knockout mice exhibit an abnormal immunological phenotype.

Flourescence-activated Cell-sorting (FACS) Analysis

Procedure:

FACS analysis of immune cell composition from peripheral blood was performed including CD4, CD8 and T cell receptor to evaluate T lymphocytes, CD19 for B lymphocytes, CD45 as a leukocyte marker and pan NK for natural killer cells. The FACS analysis was carried out on 2 wild type and 6 homozygous mice and included cells derived from thymus, spleen, bone marrow and lymph node.

In these studies, analyzed cells were isolated from thymus, peripheral blood, spleen, bone marrow and lymph nodes. Flow cytometry was designed to determine the relative proportions of CD4 and CD8 positive T cells, B cells, NK cells and monocytes in the mononuclear cell population. A Becton-Dickinson FACSCalibur 3-laser FACS machine was used to assess immune status. For Phenotypic Assays and Screening, this machine records CD4+/CD8−, CD8+/CD4−, NK, B cell and monocyte numbers in addition to the CD4+/CD8+ratio. The mononuclear cell profile was derived by staining a single sample of lysed peripheral blood from each mouse with a panel of six lineage-specific antibodies: CD45 PerCP, anti-TCRb APC, CD4 PE, CD8 FITC, pan-NK PE, and CD19 FITC. The two FITC and PE labeled antibodies stain mutually exclusive cell types. The samples were analyzed using a Becton Dickinson FACSCalibur flow cytometer with CellQuest software.

Results:

The (−/−) mice exhibited an altered distribution of leukocyte subsets in the peripheral blood, characterized by an increased mean percentage of B cells and decreased mean percentages of CD4 and CD8 cells in the cell population when compared with their (+/+) littermates and the historical means.

Thus, knocking out the gene which encodes PRO537 polypeptides causes a decrease in the T cell population as well as causing an increase in the B cell population. From these observations, PRO537 polypeptides or the gene encoding PRO537 appears to act as a negative regulator of B cell proliferation. Thus, antagonists or inhibitors of PRO537 polypeptides would be beneficial in enhancing B cell proliferation and depressing T cell proliferation.

(c) Bone Metabolism & Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Bone MicroCT Analysis:

Procedure: MicroCT was also used to get very sensitive measurements of BMD. One vertebra and 1 femur were taken from a cohort of 4 wild type and 8 homozygous mice. Measurements were taken of lumbar 5 vertebra trabecular bone volume, trabecular thickness, connectivity density and midshaft femur total bone area and cortical thickness. The μCT40 scans provided detailed information on bone mass and architecture. Multiple bones were placed into sample holders and scanned automatically. Instrument software was used to select regions of interest for analysis. Trabecular bone parameters were analyzed in the fifth lumbar vertebrae (LV5) at 16 micrometer resolution and cortical bone parameters were analyzed in the femur midshaft at a resolution of 20 micrometers.

CAT-Scan Protocol:

Mice were injected with a CT contrast agent, Omnipaque 300 (Nycomed Amershan, 300 mg of iodine per ml, 0.25 ml per animal, or 2.50-3.75 g iodine/kg of body weight) intraperitoneally. After resting in the cage for ˜10 minutes, the mouse was then sedated by intraperitoneal injection of Avertin (1.25% 2,2,2,-tribromoethanol, 20 ml/kg body weight). A CAT-scan was performed using a MicroCAT scanner (ImTek, Inc.) with the anesthetized animal lying prone on the test bed. Three dimensional images were reconstructed by the Feldkamp algorithm in a cluster of workstations using an ImTek 3D RECON software.

Results:

1. DEXA: The male (−/−) mice exhibited increased mean volumetric bone mineral density and bone mineral density in total body and femur when compared with their gender-matched (+/+) littermates and the historical means.
2. MicroCT: The male (−/−) mice exhibited increased mean femoral mid-shaft cross-sectional area when compared with their gender-matched (+/+) littermates and the historical means.
3. CAT-Scan: All 3 (−/−) mice available for analysis (M−101, M-110, and F-186) exhibited grossly increased gastrointestinal content suggesting impaired gastrointestinal motility in the mutants. However, no signs of obstruction were observed.

In summary, the (−/−) mice exhibited increased mean volumetric bone mineral density and bone mineral density in total body and femur, and increased total body and femoral bone mineral density when compared with their gender-matched (+/+) littermates. These results indicate that the knockout mutant phenotype is associated with such bone abnormalities as osteopetrosis. Osteopetrosis is a condition characterized by abnormal thickening and hardening of bone and abnormal fragility of the bones. As such, PRO537 polypeptides or agonists thereof would be beneficial for the treatment of osteopetrosis or other osteo-related diseases. On the other hand, inhibitors or antagonists of PRO537 polypeptides would be useful in bone healing. CAT-Scan results showed impaired GI motility which could be associated with the opioid receptor axis in the GI tract.

70.13. Generation and Analysis of Mice Comprising DNA49647-1398 (UNQ386) Gene Disruptions

In these knockout experiments, the gene encoding PRO718 polypeptides (designated as DNA49647-1398) (UNQ386) was disrupted. The gene specific information for these studies is as follows: the mutated mouse gene corresponds to nucleotide reference: NM_{—}027935 Mus musculus RIKEN cDNA 3200001F09 gene (3200001F09Rik); protein reference: Q9CXL1 ACCESSION:Q9CXL1 NID: Mus musculus (Mouse). 3200001F09RIK PROTEIN; the human gene sequence reference: NM_{—}014313 ACCESSION:NM_{—}014313 NID: gi 20357549 ref NM_{—}014313.2 Homo sapiens small membrane protein 1 (SMP1); the human protein sequence corresponds to reference: 095807 ACCESSION:095807 NID: Homo sapiens (Human). SMALL MEMBRANE PROTEIN 1.

The mouse gene of interest is RIKEN cDNA 3200001F09 gene, ortholog of human SMP1 (small membrane protein 1). Aliases include CAM, Smp1, and small membrane protein 1.

SMP1 is putative membrane protein of 157 amino acids, containing a signal peptide and four transmembrane segments. The protein is ubiquitously expressed and appears to be located in the cytoplasm. The function of this protein is not known (Kumada et al, Gene 299(1-2):165-72 (2002); Wagner and Flegel, Blood 95(12):3662-8 (2000); Reboul et al, Genome Res 9(3):242-50 (1999)).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 18 | 44 | 24 | 86 |
| Expected | 21.5 | 43 | 21.5 | 86 |

Chi-Sq.=2.02 Significance=0.36421898 (hom/n)=0.29 Avg. Litter Size=9

### Mutation Information

Mutation Type: Homologous Recombination (standard)
Description: Coding exon 1 and the adjacent 5-prime noncoding exon were targeted (NCBI accession NM_{—}027935. 1).
1. Wild-type Expression Panel: Expression of the target gene was detected in all 13 adult tissue samples tested by RT-PCR.
2. QC Expression: Disruption of the target gene was confirmed by Southern hybridization analysis.

70.13.1. Phenotypic Analysis (for Disrupted Gene: DNA49647-1398 (UNQ386)

(a) Overall Phenotypic Summary:

Mutation of the gene encoding the ortholog of human small membrane protein 1 (SMP1) resulted in anemia in (−/−) mice. The (−/−) mice also exhibited an increased TNF-alpha response to LPS challenge and decreased mean serum IgG2a levels. The homozygous mutant mice exhibited signs of anemia when compared with their wild-type littermates and the historical means. Blood chemistry revealed increased serum glucose levels and urinary ketone bodies in the (−/−) mice. The female (−/−) mice also exhibited increased mean bone mineral density-related measurements. Disruption of the target gene was confirmed by Southern hybridization analysis.

(b) Immunology Phenotypic Analysis

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

T lymphocytes (T cells) are an important component of a mammalian immune response. T cells recognize antigens which are associated with a self-molecule encoded by genes within the major histocompatibility complex (MHC). The antigen may be displayed together with MHC molecules on the surface of antigen presenting cells, virus infected cells, cancer cells, grafts, etc. The T cell system eliminates these altered cells which pose a health threat to the host mammal. T cells include helper T cells and cytotoxic T cells. Helper T cells proliferate extensively following recognition of an antigen-MHC complex on an antigen presenting cell. Helper T cells also secrete a variety of cytokines, i.e., lymphokines, which play a central role in the activation of B cells, cytotoxic T cells and a variety of other cells which participate in the immune response.

In many immune responses, inflammatory cells infiltrate the site of injury or infection. The migrating cells may be neutrophilic, eosinophilic, monocytic or lymphocytic as can be determined by histologic examination of the affected tissues. Current Protocols in Immunology, ed. John E. Coligan, 1994, John Wiley & Sons, Inc.

Many immune related diseases are known and have been extensively studied. Such diseases include immune-mediated inflammatory diseases (such as rheumatoid arthritis, immune mediated renal disease, hepatobiliary diseases, inflammatory bowel disease (IBD), psoriasis, and asthma), non-immune-mediated inflammatory diseases, infectious diseases, immunodeficiency diseases, neoplasia, and graft rejection, etc. In the area of immunology, targets were identified for the treatment of inflammation and inflammatory disorders.

In the area of immunology, targets have been identified herein for the treatment of inflammation and inflammatory disorders. Immune related diseases, in one instance, could be treated by suppressing the immune response. Using neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

The following tests were performed:

(1) Acute Phase Response:

Test Description: Bacterial lipopolysaccharide (LPS) is an endotoxin, and as such is a potent inducer of an acute phase response and systemic inflammation. The Level I LPS mice were injected intraperitoneally (i.p.) with a sublethal dose of LPS in 200 μL sterile saline using a 26 gauge needle. The doses were based on the average weight of the mice tested at 1 μg/g body weight 3 hours after injection; a 100u1 blood sample was then taken and analyzed for the presence of TNFa, MCP-1, and IL-6 on the FACSCalibur instrument.

Results:

The (−/−) mice exhibited an increased mean serum TNF-alpha response to LPS challenge when compared with their (+/+) littermates and the historical mean.

In summary, the LPS endotoxin challenge demonstrated that knockout mice deficient in the gene encoding PRO718 polypeptides exhibit immunological abnormalities when compared with their wild-type littermates. In particular, the mutant mice exhibited an increased ability to elicit an immunological response (TNF-alpha production) when challenged with the LPS endotoxin indicating a proinflammatory response. TNF-alpha is an important inflammatory mediator. In addition, TNF-alpha plays a critical role in inducing the acute phase response and systemic inflammation. TNF-alpha can substitute for the membrane-bound signal in macrophage activation (thus serving as an effector molecule). This suggests that inhibitors or antagonists to PRO718 polypeptides would stimulate the immune system and would find utility in the cases wherein this effect would be beneficial to the individual such as in the case of leukemia, and other types of cancer, and in immunocompromised patients, such as AIDS sufferers. Accordingly, PRO718 polypeptides or agonists thereof would be useful in inhibiting the immune response and would be useful candidates for suppressing harmful immune responses, e.g. in the case of graft rejection or graft-versus-host diseases.

(2) Hematology Analysis:

Test Description: Blood tests are carried out by Abbott's Cell-Dyn 3500R, an automated hematology analyzer. Some of its features include a five-part WBC differential. ‘Patient’ reports can cover over 22 parameters in all.

Results:

The (−/−) mice exhibited a decreased mean total red blood cell count, hemoglobin level, and hematocrit and an increase in corpuscular volume when compared with their (+/+) littermates and the historical means.

These results are related to a phenotype associated with anemia. Thus, PRO718 polypeptides, agonists thereof or the encoding gene for PRO718 polypeptides must be essential for normal red blood cell production and as such would be useful in the treatment of blood disorders associated with anemia or a low hematocrit.

(3) Serum Immunoglobulin Isotyping Assay:

The Serum Immunoglobulin Isotyping Assay is performed using a Cytometric Bead Array (CBA) kit. This assay is used to rapidly identify the heavy and light chain isotypes of a mouse monoclonal antibody in a single sample. The values expressed are “relative fluorescence units” and are based on the detection of kappa light chains.

Any value <6 is not significant.

Results:

The serum immunoglobulin isotyping assay showed decreased or reduced levels of IgG2a in the homozygous (−/−) mice compared to their gender-matched littermate (+/+) controls.

The serum immunoglobulin isotyping assay revealed that homozygous adults exhibited decreased serum IgG2a levels. Thus, homozygotes showed an abnormally low serum immunoglobulins compared with the (+/+) littermates. Thus, the gene encoding PRO718 polypeptides is essential for making immunoglobulins (or gamma globulins). Likewise, IgG2a immunoglobulins have neutralization effects and to a lesser extent are important for activation of the complement system. These immunological abnormalities suggest that PRO718 polypeptides or agonists thereof would be useful in stimulating the immune system (such as T cell proliferation) and would find utility in the cases wherein this effect would be beneficial to the individual such as in the case of leukemia, and other types of cancer, and in immunocompromised patients, such as AIDS sufferers. Accordingly, inhibitors (antagonists) of PRO718 polypeptides would inhibit the immune response and would be useful candidates for suppressing harmful immune responses, e.g. in the case of graft rejection or graft-versus-host diseases.

(c) Phenotypic Analysis: Metabolism-Blood Chemistry

In the area of metabolism, targets may be identified for the treatment of diabetes. Blood chemistry phenotypic analysis includes blood glucose measurements. The COBAS Integra 400 (mfr: Roche) was used for running blood chemistry tests on the mice. In the area of metabolism, targets may be identified for the treatment of diabetes.

Results:

Both the male and female (−/−) mice exhibited increased mean serum glucose levels when compared with their gender-matched (+/+) littermates and the historical means. However, Glucose Tolerance testing was normal.

As summarized above, the (−/−) mice exhibited notably increased mean serum glucose levels suggesting abnormal glucose metabolism or a pre-diabetic condition. Thus, mutant mice deficient in the PRO718gene can serve as a model for cardiovascular disease associated with abnormal glucose metabolism. PRO718 polypeptides or its encoding gene would be useful in regulating normal blood glucose levels. Thus, PRO718 polypeptides or agonists thereof would be useful in the treatment of such cardiovascular diseases as hypertension, atherosclerosis, heart failure, stroke, various coronary diseases, or diabetes.

Urinalysis

Description:

The routine urinalysis is a screening test done to provide a general evaluation of the renal/urinary system. The characteristics for which urine is routinely examined includes tests for protein, glucose, ketones, blood, bilirubin, urobilinogen, nitrate and leukocyte esterase, as well as pH and specific gravity.

Results:

Ketonuria was observed in the mutant homozygous (−/−) and heterozygous (+/−) mice. Thus, the mutant (−/−) and (+/−) mice showed an abnormal presence of ketone bodies which is usually associated with abnormal glucose metabolism and diabetes.

(d) Bone Metabolism & Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Results:

1. DEXA: The female (−/−) mice exhibited increased mean bone mineral density-related measurements (total body vBMD, total body BMD and femur BMD) when compared with their gender-matched (+/+) littermates and the historical means.

In summary, the (−/−) mice exhibited increased mean bone mineral density-related measurements when compared with their gender-matched (+/+) littermates. These results indicate that the knockout mutant phenotype is associated with such bone abnormalities as osteopetrosis. Osteopetrosis is a condition characterized by abnormal thickening and hardening of bone and abnormal fragility of the bones. As such, PRO718 polypeptides or agonists thereof would be beneficial for the treatment of osteopetrosis or other osteo-related diseases. On the other hand, inhibitors or antagonists of PRO718 polypeptides would be useful in bone healing.

70.14. Generation and Analysis of Mice Comprising DNA48303-2829 (UNQ411) Gene Disruptions

In these knockout experiments, the gene encoding PRO773 polypeptides (designated as DNA48303-2829) (UNQ411) was disrupted. The gene specific information for these studies is as follows: the mutated mouse gene corresponds to nucleotide reference: NM_{—}080434 Mus musculus apolipoprotein A-V (Apoa5); protein reference: Q8C7G5 ACCESSION:Q8C7G5 NID: Mus musculus (Mouse). Mus musculus adult male liver tumor cDNA, RIKEN full-length enriched library, clone: C730033H22 product: apolipoprotein A-V, full insert sequence; the human gene sequence reference: NM_{—}052968 ACCESSION:NM_{—}052968 NID: gi 22091457 refNM_{—}052968.2 Homo sapiens apolipoprotein A-V (APOA5); the human protein sequence corresponds to reference: Q9UBJ3 ACCESSION:Q9UBJ3 NID: Homo sapiens (Human). Regeneration associated protein 3.

The mouse gene of interest is Apoa5 (apolipoprotein A-V), ortholog of human APOA5. Aliases include RAP3, Apoav, 1300007O05Rik, APOA-V, apolipoprotein A5, apolipoprotein AV, and regeneration-associated protein 3.

APOA5 is an apolipoprotein expressed primarily in liver that lowers plasma triglyceride levels by a mechanism that is not clearly understood (Pennacchio et al, Science 294(5540):169-73 (2001); van der Vliet et al, J Biol Chem 276(48):44512-20 (2001); van der Vliet et al, Biochem Biophys Res Commun 295(5):1156-9 (2002)). APOA5 interacts with high-density lipoprotein (HDL), very low-density lipoprotein (VLDL), and lipoprotein lipase. Moreover, by interacting with lipoprotein lipase, APOA5 stimulates lipase activity. The increase in lipoprotein lipase activity is likely to decrease VLDL size and increase triglyceride turnover and VLDL clearance, thereby lowering plasma triglycerides (Fruchart-Najib et al, Biochem Biophys Res Commun 319(2):397-404 (2004); Schaap et al, J Biol Chem 279(27):27941-7 (2004)). APOA5 is concentrated on the membrane of the endoplasmic reticulum and is poorly secreted when expressed in COS cells. Moreover, the concentration of APOA5 in plasma is much lower than that of other apolipoproteins, suggesting that the function of APOA5 is not primarily extracellular. Intracellular APOA5 may lower plasma triglycerides by impeding triglyceride-rich particle assembly in liver (Weinberg et al, J Biol Chem 278(36):34438-44 (2003)).

The physiological role of APOA5 has been investigated by Pennacchio and colleagues (Science 294(5540):169-73 (2001) using APOA5-null mice and by Pennacchio and colleagues (2001), as well as several others (van der Vliet et al, Biochem Biophys Res Commun 295(5):1156-9 (2002); Fruchart-Najib et al, Biochem Biophys Res Commun 319(2):397-404 (2004); Schaap et al, J Biol Chem 279(27):27941-7 (2004)), using mice expressing human APOA5. They showed that plasma triglycerides were four times higher in APOA5-null mice than in wild-type mice and that plasma triglyceride levels of mice expressing human APOA5 were one-third that of wild-type mice. Moreover, Pennacchio and colleagues, as well as others (OMIM 606368), found that mutations in the APOA5 gene were associated with plasma triglyceride levels. They concluded that APOA5 is an important determinant of plasma triglyceride levels.

Targeted or gene trap mutations are generated in strain 129SvEv′-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 17 | 44 | 19 | 80 |
| Expected | 20 | 40 | 20 | 80 |

Chi-Sq.=1.54 Significance=0.46301308 (hom/n)=0.26 Avg. Litter Size=8

### Mutation Information

Mutation Type: Homologous Recombination (standard)
Description: Coding exons 1 through 3 were targeted (NCBI accession NM_{—}080434.2).
1. Wild-type Expression Panel: Expression of the target gene was detected in eye, thymus, kidney, and liver among the 13 adult tissue samples tested by RT-PCR.
2. QC Expression: Disruption of the target gene was confirmed by Southern hybridization analysis.

70.14.1. Phenotypic Analysis (for Disrupted Gene: DNA48303-2829 (UNQ411)

(a) Overall Phenotypic Summary:

Mutation of the gene encoding the ortholog of human apolipoprotein A-V (APOA5) resulted in blood chemistry abnormalities in (−/−) mice. Both the male and female homozygous mutant mice exhibited notably increased cholesterol and mean serum triglyceride levels when compared with their gender-matched wild-type littermates and the historical means. The female (−/−) mice also showed decreased total tissue mass (TTM) and vertebrae bone mineral density (BMD) measurements as well as decreased trabecular number. Disruption of the target gene was confirmed by Southern hybridization analysis.

(b) Phenotypic Analysis: Cardiology

In the area of cardiovascular biology, targets were identified herein for the treatment of hypertension, atherosclerosis, heart failure, stroke, various coronary artery diseases, dyslipidemias such as high cholesterol (hypercholesterolemia) and elevated serum triglycerides (hypertriglyceridemia), diabetes and/or obesity. The phenotypic tests included the measurement of serum cholesterol and triglycerides.

Blood Lipids

Procedure: A cohort of 4 wild type, 4 heterozygous and 8 homozygous mice were tested in this assay. High cholesterol levels and increased triglyceride blood levels are recognized risk factors in the development of cardiovascular disease and/or diabetes. Measuring blood lipids facilitates the finding of biological switches that regulate blood lipid levels Inhibition of factors which elevate blood lipid levels may be useful for reducing the risk for cardiovascular disease. In these blood chemistry tests, measurements were recorded using the COBAS Integra 400 (mfr: Roche).

Results:

1. Blood Chemistry: Both the male and female (−/−) mice exhibited notably increased mean serum cholesterol levels (>3 standard deviations above the mean) and mean serum triglyceride levels (6×(males) and 4×(females) above the mean) when compared with their gender-matched (+/+) littermates and the historical means.

As summarized above, the (−/−) mice exhibited notably increased mean serum cholesterol and triglyceride levels when compared with their gender-matched (+/+) littermates and the historical means. Thus, mutant mice deficient in the PRO773 gene can serve as a model for cardiovascular disease. PRO733 polypeptides or its encoding gene would be useful in regulating blood lipids such as cholesterol and triglycerides. Thus, PRO733 polypeptides or agonists thereof would be useful in the treatment of such cardiovascular diseases as hypertension, atherosclerosis, heart failure, stroke, various coronary diseases, hypercholesterolemia, hypertriglyceridemia, diabetes and/or obesity.

(c) Bone Metabolism & Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Bone micro CT Analysis:

Procedure: MicroCT was also used to get very sensitive measurements of BMD. One vertebra and 1 femur were taken from a cohort of 4 wild type and 8 homozygous mice. Measurements were taken of lumbar 5 vertebra trabecular bone volume, trabecular thickness, connectivity density and midshaft femur total bone area and cortical thickness. The μCT40 scans provided detailed information on bone mass and architecture. Multiple bones were placed into sample holders and scanned automatically. Instrument software was used to select regions of interest for analysis. Trabecular bone parameters were analyzed in the fifth lumbar vertebrae (LV5) at 16 micrometer resolution and cortical bone parameters were analyzed in the femur midshaft at a resolution of 20 micrometers.

Results:

1. DEXA: Female knockout (−/−) mice exhibited a decreased total tissue mass and vertebrae bone mineral density compared to wildtype littermates and the historical means.
2. MicroCT: The (−/−) mice exhibited decreased trabecular number compared the wildtype (+/+) littermates.

The (−/−) mice analyzed by DEXA and bone micro CT analysis exhibited decreased bone measurements when compared with their (+/+) littermates, suggestive of abnormal bone disorders. The (−/−) mice exhibited a negative bone phenotype with abnormal decreased bone measurements reflective of bone metabolic disorders. The negative bone phenotype indicates that PRO773 polypeptides or agonists thereof would be useful for maintaining bone homeostasis. In addition, PRO773 polypeptides would be useful in bone healing or for the treatment of arthritis or osteoporosis, whereas antagonists (or inhibitors) of PRO773 polypeptides or its encoding gene would lead to abnormal or pathological bone disorders including inflammatory diseases associated with abnormal bone metabolism including arthritis, osteoporosis and osteopenia.

70.15. Generation and Analysis of Mice Comprising DNA60614 (UNQ421) Gene Disruptions

In these knockout experiments, the gene encoding PRO860 polypeptides (designated as DNA60614) (UNQ421) was disrupted. The gene specific information for these studies is as follows: the mutated mouse gene corresponds to nucleotide reference: NM_{—}028783 Mus musculus roundabout homolog 4 (Drosophila) (Robo4); protein reference: NP_{—}083059 roundabout homolog 4; Magic roundabout; roundabout homolog 4 (Drosophilia) [Mus musculus] gi|26334430|dbj|BAB23506.2| unnamed protein product [Mus musculus]; the human gene sequence reference: NM_{—}019055 ACCESSION:NM_{—}019055 NID: gi 17511434 refNM_{—}019055.4 Homo sapiens roundabout homolog 4, magic roundabout (Drosophila) (ROBO4); the human protein sequence corresponds to reference: Q8WZ75 ACCESSION:Q8WZ75 NID: Homo sapiens (Human). MAGIC ROUNDABOUT.

The mouse gene of interest is Robo4 (roundabout homolog 4 [Drosophila]), ortholog of human ROBO4 (roundabout homolog 4, magic roundabout [Drosophila]). Aliases include 1200012D01Rik, Magic roundabout, and FLJ20798.

ROBO4 is a type I plasma membrane protein expressed primarily in endothelial cells that likely functions as a receptor. Activation of ROBO4 with SLIT proteins or possibly other ligands inhibits vascular endothelial cell migration, tube formation, and angiogenesis, suggesting that ROBO4 plays a role in vascular sprouting (Huminiecki et al, Genomics 79(4):547-52 (2002); Park et al, Dev Biol 261(1):251-67 (2003)).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 16 | 47 | 18 | 81 |
| Expected | 20.25 | 40.5 | 20.25 | 81 |

Chi-Sq.=2.02 Significance=0.3642 1898 (hom/n)=0.22 Avg. Litter Size=7

### Mutation Information

Mutation Type: Homologous Recombination (standard)
Description: Coding exons 1 through 3 were targeted (NCBI accession NM_{—}028783.2).
1. Wild-type Expression Panel: Expression of the target gene was detected in all 13 adult tissue samples tested by RT-PCR, except skeletal muscle, bone, and adipose.
2. QC Expression: Disruption of the target gene was confirmed by Southern hybridization analysis.

70.15.1. Phenotypic Analysis (for Disrupted Gene: DNA60614 (UNQ421)

(a) Overall Phenotypic Summary:

Mutation of the gene encoding the ortholog of human roundabout homolog 4, magic roundabout (Drosophila) (ROBO4) resulted in an increased anxiety-related response in male (−/−) mice during open field testing. Blood chemistry results showed increased levels of both cholesterol and triglycerides as well as elevated levels of phosphorous. Disruption of the target gene was confirmed by Southern hybridization analysis.

(b) Expression

UNQ421 is specifically expressed in the vascular endothelium of murine embryos (E10.5 trunk).

(c) Phenotypic Analysis: CNS/Neurology

In the area of neurology, analysis focused herein on identifying in vivo validated targets for the treatment of neurological and psychiatric disorders including depression, generalized anxiety disorders, attention deficit hyperactivity disorder, obsessive compulsive disorder, schizophrenia, cognitive disorders, hyperalgesia and sensory disorders. Neurological disorders include the category defined as “anxiety disorders” which include but are not limited to: mild to moderate anxiety, anxiety disorder due to a general medical condition, anxiety disorder not otherwise specified, generalized anxiety disorder, panic attack, panic disorder with agoraphobia, panic disorder without agoraphobia, posttraumatic stress disorder, social phobia, specific phobia, substance-induced anxiety disorder, acute alcohol withdrawal, obsessive compulsive disorder, agoraphobia, bipolar disorder I or II, bipolar disorder not otherwise specified, cyclothymic disorder, depressive disorder, major depressive disorder, mood disorder, substance-induced mood disorder. In addition, anxiety disorders may apply to personality disorders including but not limited to the following types: paranoid, antisocial, avoidant behavior, borderline personality disorders, dependent, histronic, narcissistic, obsessive-compulsive, schizoid, and schizotypal.

Procedure:

Behavioral screens were performed on a cohort of 4 wild type, 4 heterozygous and 8 homozygous mice. All behavioral tests were done between 12 and 16 weeks of age unless reduced viability necessitates earlier testing. These tests included open field to measure anxiety, activity levels and exploration.

Open Field Test:

Several targets of known drugs have exhibited phenotypes in the open field test. These include knockouts of the seratonin transporter, the dopamine transporter (Giros et al., Nature. 1996 Feb. 15; 379(6566):606-12), and the GABA receptor (Homanics et al., Proc Natl Acad Sci USA. 1997 Apr. 15; 94(8):4143-8). An automated open-field assay was customized to address changes related to affective state and exploratory patterns related to learning. First, the field (40×40 cm) was selected to be relatively large for a mouse, thus designed to pick up changes in locomotor activity associated with exploration. In addition, there were 4 holes in the floor to allow for nose-poking, an activity specifically related to exploration. Several factors were also designed to heighten the affective state associated with this test. The open-field test is the first experimental procedure in which the mice are tested, and the measurements that were taken were the subjects' first experience with the chamber. In addition, the open-field was brightly lit. All these factors will heighten the natural anxiety associated with novel and open spaces. The pattern and extent of exploratory activity, and especially the center-to-total distance traveled ratio, may then be able to discern changes related to susceptibility to anxiety or depression. A large arena (40 cm×40 cm, VersaMax animal activity monitoring system from AccuScan Instruments) with infrared beams at three different levels was used to record rearing, hole poke, and locomotor activity. The animal was placed in the center and its activity was measured for 20 minutes. Data from this test was analyzed in five, 4-minute intervals. The total distance traveled (cm), vertical movement number (rearing), number of hole pokes, and the center to total distance ratio were recorded.

The propensity for mice to exhibit normal habituation responses to a novel environment is assessed by determining the overall change in their horizontal locomotor activity across the 5 time intervals. This calculated slope of the change in activity over time is determined using normalized, rather than absolute, total distance traveled. The slope is determined from the regression line through the normalized activity at each of the 5 time intervals. Normal habituation is represented by a negative slope value.

Results:

Anxiety: The male (−/−) mice exhibited decreased median sum time-in-center during open field testing when compared with their gender-matched (+/+) littermates and the historical mean, suggesting an increased anxiety-like response in the mutants.

The (−/−) mice demonstrated a decrease median sum time-in-center at intervals 2, 3, and 5 when compared to the (+/+) mice, suggesting an increased anxiety-like response in the (−/−) mice. In summary, the open field testing revealed a phenotype associated with increased anxiety which could be associated with mild to moderate anxiety, anxiety due to a general medical condition, and/or bipolar disorders; hyperactivity; sensory disorders; obsessive-compulsive disorders, schizophrenia or a paranoid personality. Thus, PRO860 polypeptides or agonists thereof would be useful in the treatment of such neurological disorders.

(d) Phenotypic Analysis: Cardiology

In the area of cardiovascular biology, targets were identified herein for the treatment of hypertension, atherosclerosis, heart failure, stroke, various coronary artery diseases, dyslipidemias such as high cholesterol (hypercholesterolemia) and elevated serum triglycerides (hypertriglyceridemia), diabetes and/or obesity. The phenotypic tests included the measurement of serum cholesterol and triglycerides.

Blood Lipids

Procedure: A cohort of 4 wild type, 4 heterozygous and 8 homozygous mice were tested in this assay. High cholesterol levels and increased triglyceride blood levels are recognized risk factors in the development of cardiovascular disease and/or diabetes. Measuring blood lipids facilitates the finding of biological switches that regulate blood lipid levels Inhibition of factors which elevate blood lipid levels may be useful for reducing the risk for cardiovascular disease. In these blood chemistry tests, measurements were recorded using the COBAS Integra 400 (mfr: Roche).

Results:

The male (−/−) mice exhibited an increased mean serum triglyceride levels (one standard deviation>historic mean) as well as increased cholesterol levels (two standard deviations>historic mean) when compared with their gender-matched (+/+) littermates and the historical mean.

As summarized above, the (−/−) mice exhibited notably increased mean serum cholesterol and triglyceride levels when compared with their gender-matched (+/+) littermates and the historical means. Thus, mutant mice deficient in the PRO860 gene can serve as a model for cardiovascular disease. PRO860 polypeptides or its encoding gene would be useful in regulating blood lipids such as triglycerides and cholesterol Thus, PRO860 polypeptides or agonists thereof would be useful in the treatment of such cardiovascular diseases as hypertension, atherosclerosis, heart failure, stroke, various coronary diseases, hypercholesterolemia, hypertriglyceridemia, diabetes and/or obesity.

(e) Phenotypic Analysis: Metabolism-Blood Chemistry

In the area of metabolism, targets may be identified for the treatment of metabolic disorders. The COBAS Integra 400 (mfr: Roche) was used for running blood chemistry tests on the mice. In addition to measuring blood glucose levels the following blood chemistry tests are also routinely performed: Alkaline Phosphatase; Alanine Amino-Transferase; Albumin; Bilirubin; Phosphorous; Creatinine; BUN=Blood Urea Nitrogen; Calcium; Uric Acid; Sodium; Potassium; and Chloride.

Procedure: A cohort of 2 wild type and 4 homozygous mice were used in this assay. The COBAS Integra 400 (mfr: Roche) was used for running blood chemistry tests on the mice.

Results:

The mutant (−/−) mice exhibited elevated levels of phosphorus (about two standard deviations above the historic means). Although the measurements of phosphorus are abnormal, the mutant (−/−) mice did not exhibit typical bone measurements which would could be associated with this observation.

70.16. Generation and Analysis of Mice Comprising DNA50919-1361 (UNQ438) Gene Disruptions

In these knockout experiments, the gene encoding PRO871 polypeptides (designated as DNA50919-1361) (UNQ438) was disrupted. The gene specific information for these studies is as follows: the mutated mouse gene corresponds to nucleotide reference: XM_{—}127535 Mus musculus serologically defined colon cancer antigen 10 (Sdccag10); protein reference: XP_{—}127535 serologically defined colon cancer antigen 10 [Mus musculus]; the human gene sequence reference: AY358569 Homo sapiens clone DNA50919 SDCCAG10 (UNQ438); the human protein sequence corresponds to reference: Q6UX04 ACCESSION:Q6UX04 NID: Homo sapiens (Human). SDCCAG10.

The mouse gene of interest is Sdccag10 (serologically defined colon cancer antigen 10), ortholog of human SDCCAG10. Aliases include NY—CO-10 and 3110009E13Rik.

SDCCAG10 is a putative nuclear peptidyl-prolyl cis-trans isomerase, catalyzing the cis-trans isomerization of proline imidic peptide bonds. The protein contains a cyclophilin type peptidyl-prolyl cis-trans isomerase domain (Pfam accession PF00160) and a bipartite nuclear localization signal. SDCCAG10 is likely to be involved in protein folding SDCCAG10 is also a tumor antigen that appears in some colon cancers (Scanlan et al, Int J Cancer 76(5):652-8 (1998)).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 37 | 78 | 9 | 124 |
| Expected | 31 | 62 | 31 | 124 |

Chi-Sq.=20.9 Significance=2.894828E-5 (hom/n)=0.07 Avg. Litter Size=4

### Mutation Information

Mutation Type: Homologous Recombination (standard)
Description: Coding exon 1 was targeted (NCBI accession BC025437).
1. Wild-type Expression Panel: Expression of the target gene was detected in embryonic stem (ES) cells and in all 13 adult tissue samples tested by RT-PCR, except skeletal muscle and bone.
2. QC Expression: Disruption of the target gene was confirmed by Southern hybridization analysis.

70.16.1. Phenotypic Analysis (for Disrupted Gene: DNA50919-1361 (UNQ438)

(a) Overall Phenotypic Summary:

Mutation of the gene encoding the ortholog of human serologically defined colon cancer antigen 10 (SDCCAG10) resulted in greatly reduced viability of (−/−) mutants. The single surviving (−/−) mouse exhibited growth retardation and retinal depigmentation. Numerous neurological, immunological, and blood chemistry abnormalities were also observed in the (−/−) mouse. Disruption of the target gene was confirmed by Southern hybridization analysis.

(b) Pathology

Microscopic Observations: At day 12.5, there were 46 embryos observed: 8 (−/−) embryos, 21 (+/−) embryos, 7 (+/+) embryos, 5 resorption moles, and 5 inconclusive. The (−/−) embryos were generally smaller than their (+/+) littermates, but no other developmental abnormalities were detected by gross or histological examination. Gene Expression LacZ activity was not detected in the panel of tissues by immunohistochemical analysis.

(c) Genetics:

Greatly reduced viability of the (−/−) mice was observed. All but one of the (−/−) mutants identified were embryonic samples.

(d) Immunology Phenotypic Analysis

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

T lymphocytes (T cells) are an important component of a mammalian immune response. T cells recognize antigens which are associated with a self-molecule encoded by genes within the major histocompatibility complex (MHC). The antigen may be displayed together with MHC molecules on the surface of antigen presenting cells, virus infected cells, cancer cells, grafts, etc. The T cell system eliminates these altered cells which pose a health threat to the host mammal. T cells include helper T cells and cytotoxic T cells. Helper T cells proliferate extensively following recognition of an antigen-MHC complex on an antigen presenting cell. Helper T cells also secrete a variety of cytokines, i.e., lymphokines, which play a central role in the activation of B cells, cytotoxic T cells and a variety of other cells which participate in the immune response.

In many immune responses, inflammatory cells infiltrate the site of injury or infection. The migrating cells may be neutrophilic, eosinophilic, monocytic or lymphocytic as can be determined by histologic examination of the affected tissues. Current Protocols in Immunology, ed. John E. Coligan, 1994, John Wiley & Sons, Inc.

Many immune related diseases are known and have been extensively studied. Such diseases include immune-mediated inflammatory diseases (such as rheumatoid arthritis, immune mediated renal disease, hepatobiliary diseases, inflammatory bowel disease (IBD), psoriasis, and asthma), non-immune-mediated inflammatory diseases, infectious diseases, immunodeficiency diseases, neoplasia, and graft rejection, etc. In the area of immunology, targets were identified for the treatment of inflammation and inflammatory disorders.

In the area of immunology, targets have been identified herein for the treatment of inflammation and inflammatory disorders. Immune related diseases, in one instance, could be treated by suppressing the immune response. Using neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

The following test was performed:

Hematology Analysis:

Test Description Blood tests are carried out by Abbott's Cell-Dyn 3500R, an automated hematology analyzer. Some of its features include a five-part WBC differential. ‘Patient’ reports can cover over 22 parameters in all.

Results:

The single (−/−) mouse available for analysis (M-130) exhibited increased absolute neutrophil and monocyte counts; a decreased absolute lymphocyte count; decreased red blood cell count, hemoglobin level, hematocrit level, mean corpuscular volume, and mean corpuscular hemoglobin; and increased red blood cell distribution width and mean platelet volume. No notable differences were observed for the (+/−) mice.

These results indicate that mutant (−/−) mice have several immunological abnormalities compared with their wildtype littermates. In summary, the hematology results indicate that the homozygous mutant mice exhibited an increased neutrophils and monocyte count compared to their littermate controls indicating elevated levels of precursors of macrophages with increased phagocytic activity or ability to engulf or kill extracellular pathogens. In addition, the (−/−) mice showed a decreased absolute lymphocyte count indicative of abnormal adaptive immunity. In addition to the observation of decreased neutrophils and monocytes, the mutant (−/−) mice exhibited a phenotype associated with anemia. Thus, PRO871 polypeptides, agonists thereof or the encoding gene for PRO871 polypeptides must be essential for normal red blood cell production and as such would be useful in the treatment of blood disorders associated with anemia or a low hematocrit. In addition, PRO871 polypeptides must be essential for maintaining a normal immunological profile especially for adaptive immunity

(e) Cardiovascular Phenotypic Analysis:

In the area of cardiovascular biology, phenotypic testing was performed to identify potential targets for the treatment of cardiovascular, endothelial or angiogenic disorders. One such phenotypic test included optic fundus photography and angiography to determine the retinal arteriovenous ratio (A/V ratio) in order to flag various eye abnormalities. An abnormal A/V ratio signals such systemic diseases or disorders that may be related to the vascular disease of hypertension (and any disease that causes hypertension, e.g. atherosclerosis), diabetes or other ocular diseases corresponding to ophthalmological disorders. Such eye abnormalities may include but are not limited to the following: retinal abnormality is retinal dysplasia, various retinopathies, restenosis, retinal artery obstruction or occlusion; retinal degeneration causing secondary atrophy of the retinal vasculature, retinitis pigmentosa, macular dystrophies, Stargardt's disease, congenital stationary night blindness, choroideremia, gyrate atrophy, Leber's congenital amaurosis, retinoschisis disorders, Wagner's syndrome, Usher syndromes, Zellweger syndrome, Saldino-Mainzer syndrome, Senior-Loken syndrome, Bardet-Biedl syndrome, Alport's syndrome, Alstom's syndrome, Cockayne's syndrome, dysplaisa spondyloepiphysaria congentia, Flynn-Aird syndrome, Friedreich ataxia, Hallgren syndrome, Marshall syndrome, Albers-Schnoberg disease, Refsum's disease, Kearns-Sayre syndrome, Waardenburg's syndrome, Alagile syndrome, myotonic dystrophy, olivopontocerebellar atrophy, Pierre-Marie dunsdrome, Stickler syndrome, carotinemeia, cystinosis, Wolfram syndrome, Bassen-Kornzweig syndrome, abetalipoproteinemia, incontinentia pigmenti, Batten's disease, mucopolysaccharidoses, homocystinuria, or mannosidosis.

Procedure: A cohort of wild type and homozygous mice were tested in this assay. Optic fundus photography was performed on conscious animals using a Kowa Genesis small animal fundus camera modified according to Hawes and coauthors (Hawes et al., 1999 Molecular Vision 1999; 5:22). Intra-peritoneal injection of fluorescein permitted the acquisition of direct light fundus images and fluorescent angiograms for each examination. In addition to direct ophthalmological changes, this test can detect retinal changes associated with systemic diseases such as diabetes and atherosclerosis or other retinal abnormalities. Pictures were provided of the optic fundus under normal light. The angiographic pictures allowed examination of the arteries and veins of the eye. In addition an artery to vein (A/V) ratio was determined for the eye.

Ophthalmology analysis was performed on generated F2 wild type mice and homozygous mutant progeny using the protocol described above. Specifically, the A/V ratio was measured and calculated according to the fundus images with Kowa COMIT+ software. This test takes color photographs through a dilated pupil: the images help in detecting and classifying many diseases. The artery to vein ratio (AN) is the ratio of the artery diameter to the vein diameter (measured before the bifurcation of the vessels). Many diseases will influence the ratio, i.e., diabetes, cardiovascular disorders, papilledema, optic atrophy or other eye abnormalities such as retinal degeneration (known as retinitis pigmentosa) or retinal dysplasia, vision problems or blindness. Thus, phenotypic observations which result in an increased artery-to-vein ratio in homozygous (−/−) and heterozygous (+/−) mutant progeny compared to wildtype (+/+) littermates would be indicative of such pathological conditions.

Results:

Fundus: The single (−/−) mouse available for analysis (M-130) exhibited depigmentation spots and patches on the center retinal regions and along the retinal vessels of both eyes. Such abnormalities are associated with retinal degeneration.

In summary, in this study, the single (−/−) mice showed opthamological abnormalities which would lead to abnormal retinal vessels and retinal degeneration when compared with their (+/+) littermates. In summary, by knocking out the gene identified as DNA50919-1361 encoding PRO871 polypeptides, homozygous mutant progeny exhibit phenotypes which are associated with retinal artery abnormalities. Such detected retinal changes are most commonly associated with cardiovascular systemic diseases or disorders that may be related to the vascular disease of hypertension (and any disease that causes hypertension, e.g. atherosclerosis), diabetes or other ocular diseases corresponding to ophthalmological disorders such as retinal degeneration and even blindness. Thus, antagonists of PRO871 encoding genes would lead to similar pathological retinal changes, whereas agonists would be useful as therapeutic agents in the treatment of hypertension, atherosclerosis or other opthamological disorders including retinal degeneration and diseases associated with this condition (as indicated above).

(f) Phenotypic Analysis: Metabolism-Blood Chemistry/Glucose Tolerance

In the area of metabolism, targets may be identified for the treatment of diabetes. Blood chemistry phenotypic analysis includes blood glucose measurements. The COBAS Integra 400 (mfr: Roche) was used for running blood chemistry tests on the mice. In addition to measuring blood glucose levels the following blood chemistry tests are also routinely performed: Alkaline Phosphatase; Alanine Amino-Transferase; Albumin; Bilirubin; Phosphorous; Creatinine; BUN=Blood Urea Nitrogen; Calcium; Uric Acid; Sodium; Potassium; and Chloride. In the area of metabolism, targets may be identified for the treatment of diabetes. Blood chemistry phenotypic analysis includes glucose tolerance tests to measure insulin sensitivity and changes in glucose metabolism. Abnormal glucose tolerance test results may indicate but may not be limited to the following disorders or conditions: Diabetes Type 1 and Type 2, Syndrome X, various cardiovascular diseases and/or obesity.

Procedure: A cohort of wild type and homozygous mice were used in this assay. The glucose tolerance test is the standard for defining impaired glucose homeostasis in mammals. Glucose tolerance tests were performed using a Lifescan glucometer. Animals were injected IP at 2 g/kg with D-glucose delivered as a 20% solution and blood glucose levels were measured at 0, 30, 60 and 90 minutes after injection. The COBAS Integra 400 (mfr: Roche) was used for running blood chemistry tests on the mice.

Results:

Blood Glucose Levels/Glucose Tolerance Test:

1. Blood Chemistry: The single (−/−) mouse available for analysis (M-130) exhibited decreased serum glucose and serum albumin levels when compared with its (+/+) littermates and the historical means. No notable differences were observed for the (+/−) mice.
2. Glucose Tolerance Test: The (−/−) mouse exhibited a notably decreased fasting serum glucose level and enhanced glucose tolerance when compared with its gender-matched (+/+) littermates and the historical means.

In these studies the mutant (−/−) mouse showed an increased or enhanced glucose tolerance in the presence of normal fasting glucose at all 3 intervals tested when compared with their gender-matched (+/+) littermates and the historical means. In addition, hyperinsulinemia was not apparent in the (−/−) mice. Thus, knockout mice exhibited an increased insulin sensitivity or the opposite phenotypic pattern of an impaired glucose homeostasis.

Urinalysis

Description:

The routine urinalysis is a screening test done to provide a general evaluation of the renal/urinary system. The characteristics for which urine is routinely examined includes tests for protein, glucose, ketones, blood, bilirubin, urobilinogen, nitrate and leukocyte esterase, as well as pH and specific gravity.

Results:

The (−/−) mouse exhibited leukocyturia and hematuria. The observation of leukocytes in the urine is consistent with the immunological observations of an abnormal leukocyte composition.

(g) Bone Metabolism & Body Diagnostics

(1) Tissue Mass & Lean Body Mass Measurements—Dexa

Dexa Analysis—Test Description:

Procedure: A cohort of wild type, heterozygous and homozygous mice were tested in this assay. Dual Energy X-ray Absorptiometry (DEXA) has been used successfully to identify changes in total tissue mass (TTM).

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI, i.e., whole body, vertebrae, and both femurs).

Body Measurements (Body Length & Weight):

Body Measurements: A measurement of body length and weight was performed at approximately 16 weeks of age.

Results:

General Observations: The single (−/−) mouse born (M-130) was smaller than its gender-matched littermates. The (−/−) mouse exhibited decreased body weight and decreased body length when compared with its gender-matched (+/+) littermates and the historical means.

(2) Bone Metabolism: Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of wild type, heterozygous and homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Bone MicroCT Analysis:

Procedure: MicroCT was also used to get very sensitive measurements of BMD. One vertebra and 1 femur were taken from a cohort of wild type and homozygous mice. Measurements were taken of lumbar 5 vertebra trabecular bone volume, trabecular thickness, connectivity density and midshaft femur total bone area and cortical thickness. The μCT40 scans provided detailed information on bone mass and architecture. Multiple bones were placed into sample holders and scanned automatically. Instrument software was used to select regions of interest for analysis. Trabecular bone parameters were analyzed in the fifth lumbar vertebrae (LV5) at 16 micrometer resolution and cortical bone parameters were analyzed in the femur midshaft at a resolution of 20 micrometers.

Results:

1. DEXA: The single male (−/−) mouse available for analysis (M-130) exhibited decreased total tissue mass and lean body mass when compared with its gender-matched (+/+) littermates and the historical means. Also, the (−/−) mouse showed decreased vertebrae bone mineral density. No notable differences were observed for the (+/−) mice.
2. MicroCT: The single male knockout (−/−) mouse tested showed reduced trabecular bone volume, number, thickness, connectivity density, and midshaft femur total area.

Body measurements and DEXA and bone microCT analysis (weight, length and total tissue mass and lean body mass indicate growth retardation in the single (−/−) mouse which is accompanied by the observation of reduced viability of (−/−) mice. The (−/−) mice analyzed by DEXA also exhibited decreased bone measurements when compared with their (+/+) littermates, suggestive of abnormal bone disorders. The (−/−) mouse exhibited a negative bone phenotype with abnormal and decreased bone measurements reflective of bone metabolic disorders. The negative bone phenotype indicates that PRO871 polypeptides or agonists thereof would be useful for maintaining bone homeostasis. In addition, PRO871 polypeptides would be useful in bone healing or for the treatment of arthritis or osteoporosis, whereas antagonists (or inhibitors) of PRO871 polypeptides or its encoding gene would lead to abnormal or pathological bone disorders including inflammatory diseases associated with abnormal bone metabolism including arthritis, osteoporosis and osteopenia.

(3) Diagnostics—Blood Pressure

Description:

Systolic blood pressure is measured via a noninvasive tail-cuff method for four days on the Visitech BP-2000 Blood Pressure Analysis System. The blood pressure is measured ten times each day for four days. The four days are then averaged to obtain a mouse's conscious systolic blood pressure.

Results:

The (−/−) mouse exhibited decreased systolic blood pressure when compared to its gender-matched (+/+) littermates and the historical mean.

70.17. Generation and Analysis of Mice Comprising DNA49819-1439 (UNQ439) Gene Disruptions

In these knockout experiments, the gene encoding PRO872 polypeptides (designated as DNA49819-1439) (UNQ439) was disrupted. The gene specific information for these studies is as follows: the mutated mouse gene corresponds to nucleotide reference: AF466400 Mus musculus hypothetical protein MMT-7 gene, complete cds; protein reference: Q8VHE7 ACCESSION:Q8VHE7 NID: Mus musculus (Mouse). Hypothetical 67.5 kDa protein; the human gene sequence reference: NM_{—}017750 ACCESSION:NM_{—}017750 NID: gi 8923274 ref NM_{—}017750.1 Homo sapiens hypothetical protein FLJ20296 (FLJ20296); the human protein sequence corresponds to reference: Q8N2H5 ACCESSION:Q8N2H₅NID: Homo sapiens (Human). Hypothetical protein FLJ90780.

The mouse gene of interest is RIKEN cDNA 0610039N19 gene, ortholog of human hypothetical protein FLJ20296 (FLJ20296).

Hypothetical protein FLJ20296 is a putative oxidoreductase. The protein contains a signal peptide, an overlapping transmembrane segment, and a phytoene dehydrogenase domain (InterPro accession IPR008151). Proteins with this domain include amine oxidases, such as monoamine oxidase and L-amino acid oxidase, and use FAD or NAD as a cosubstrate (InterPro accession IPR002937). The predicted subcellular location of hypothetical protein FLJ20296 is ambiguous. The protein may be associated with the endoplasmic reticular membrane, the mitochondrial membrane, or the plasma membrane.

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 15 | 38 | 12 | 65 |
| Expected | 16.5 | 32.5 | 16.5 | 65 |

Chi-Sq.=4.74 Significance=0.093480736 (hom/n)=0.23 Avg. Litter Size=7

### Mutation Information

Mutation Type: Homologous Recombination (standard) vDescription: Coding exons 1 through 4 were targeted (Accession: AF466400).
1. Wild-type Expression Panel: Expression of the target gene was detected in embryonic stem (ES) cells and in all 13 adult tissue samples tested by RT-PCR, except lung, skeletal muscle, and bone.
2. QC Expression: Disruption of the target gene was confirmed by Southern hybridization analysis.

70.17.1. Phenotypic Analysis (for Disrupted Gene: DNA49819-1439 (UNQ439)

(a) Overall Phenotypic Summary:

Mutation of the gene encoding the ortholog of a human hypothetical oxidoreductase enzyme resulted in increased percent total body fat and total fat mass as well as increased total tissue mass in (−/−) mice. Male (−/−) mice also exhibited an increased serum cholesterol level as well as the presence of ketone bodies in the blood. The (−/−) mutant mice exhibited immunological abnormalities. Disruption of the target gene was confirmed by Southern hybridization analysis.

(b) Phenotypic Analysis: Cardiology

In the area of cardiovascular biology, targets were identified herein for the treatment of hypertension, atherosclerosis, heart failure, stroke, various coronary artery diseases, dyslipidemias such as high cholesterol (hypercholesterolemia), diabetes and/or obesity. The phenotypic tests included the measurement of serum cholesterol.

Blood Lipids

Procedure: A cohort of 4 wild type, 4 heterozygous and 8 homozygous mice were tested in this assay. High cholesterol levels are recognized risk factors in the development of cardiovascular disease and/or diabetes. Measuring blood lipids facilitates the finding of biological switches that regulate blood lipid levels Inhibition of factors which elevate blood lipid levels may be useful for reducing the risk for cardiovascular disease. In these blood chemistry tests, measurements were recorded using the COBAS Integra 400 (mfr: Roche).

Results:

The male (−/−) mice exhibited an increased mean serum cholesterol level when compared with their gender-matched (+/+) littermates and the historical mean. Thus, mutant mice deficient in the PRO872 gene can serve as a model for cardiovascular disease. PRO872 polypeptides or its encoding gene would be useful in regulating blood lipids. Thus, PRO872 polypeptides or agonists thereof would be useful in the treatment of such cardiovascular diseases as hypertension, atherosclerosis, heart failure, stroke, various coronary diseases, hypercholesterolemia, diabetes and/or obesity.

Ketone bodies were also observed in the blood which is an indication of abnormal lipid metabolism and/or diabetes.

(c) Bone Metabolism & Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Results:

DEXA: Among the 8 (−/−) mice analyzed, 3 males and 3 females exhibited notably increased percent total body fat and total fat mass when compared with their gender-matched (+/+) littermates and the historical means. The male (−/−) mice also exhibited increased mean total tissue mass.

These studies suggest that mutant (−/−) non-human transgenic animals exhibit a negative phenotype that can be associated with obesity. Thus, PRO872 polypeptides or agonists thereof would be essential for normal growth and metabolic processes and would be especially important in the prevention and/or treatment of obesity.

(d) Immunology Phenotypic Analysis

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

T lymphocytes (T cells) are an important component of a mammalian immune response. T cells recognize antigens which are associated with a self-molecule encoded by genes within the major histocompatibility complex (MHC). The antigen may be displayed together with MHC molecules on the surface of antigen presenting cells, virus infected cells, cancer cells, grafts, etc. The T cell system eliminates these altered cells which pose a health threat to the host mammal. T cells include helper T cells and cytotoxic T cells. Helper T cells proliferate extensively following recognition of an antigen-MHC complex on an antigen presenting cell. Helper T cells also secrete a variety of cytokines, i.e., lymphokines, which play a central role in the activation of B cells, cytotoxic T cells and a variety of other cells which participate in the immune response.

In many immune responses, inflammatory cells infiltrate the site of injury or infection. The migrating cells may be neutrophilic, eosinophilic, monocytic or lymphocytic as can be determined by histologic examination of the affected tissues. Current Protocols in Immunology, ed. John E. Coligan, 1994, John Wiley & Sons, Inc.

Many immune related diseases are known and have been extensively studied. Such diseases include immune-mediated inflammatory diseases (such as rheumatoid arthritis, immune mediated renal disease, hepatobiliary diseases, inflammatory bowel disease (IBD), psoriasis, and asthma), non-immune-mediated inflammatory diseases, infectious diseases, immunodeficiency diseases, neoplasia, and graft rejection, etc. In the area of immunology, targets were identified for the treatment of inflammation and inflammatory disorders.

In the area of immunology, targets have been identified herein for the treatment of inflammation and inflammatory disorders. Immune related diseases, in one instance, could be treated by suppressing the immune response. Using neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

The following test was performed:

Flourescence-activated Cell-sorting (FACS) Analysis/Tissue Specific FACS

Procedure:

FACS analysis of immune cell composition from peripheral blood was performed including CD4, CD8 and T cell receptor to evaluate T lymphocytes, CD19 for B lymphocytes, CD45 as a leukocyte marker and pan NK for natural killer cells. The FACS analysis was carried out on 2 wild type and 6 homozygous mice and included cells derived from thymus, spleen, bone marrow and lymph node.

In these studies, analyzed cells were isolated from thymus, peripheral blood, spleen, bone marrow and lymph nodes. Flow cytometry was designed to determine the relative proportions of CD4 and CD8 positive T cells, B cells, NK cells and monocytes in the mononuclear cell population. A Becton-Dickinson FACSCalibur 3-laser FACS machine was used to assess immune status. For Phenotypic Assays and Screening, this machine records CD4+/CD8−, CD8+/CD4−, NK, B cell and monocyte numbers in addition to the CD4+/CD8+ratio. The mononuclear cell profile was derived by staining a single sample of lysed peripheral blood from each mouse with a panel of six lineage-specific antibodies: CD45 PerCP, anti-TCRb APC, CD4 PE, CD8 FITC, pan-NK PE, and CD19 FITC. The two FITC and PE labeled antibodies stain mutually exclusive cell types. The samples were analyzed using a Becton Dickinson FACSCalibur flow cytometer with CellQuest software.

Results:

The (−/−) mice exhibited skewed ratios of B220/med/CD23− and B220+/CD11b− low/CD23− cells after peritoneal lavage compared to their gender-matched (+/+) littermates and the historical means. Thus, antagonists or inhibitors of PRO872 polypeptides would be expected to mimic this negative phenotype which is characterized by an abnormal distribution of B cell progenitors. On the other hand, PRO872 polypeptides or agonists thereof would be expected to be useful in maintaining or producing adequate levels of B cell progenitors and would assist in the maturation of B cells which is important for the adaptive immune response.

70.18. Generation and Analysis of Mice Comprising DNA57834-1339 (UN0465) Gene Disruptions

In these knockout experiments, the gene encoding PRO813 polypeptides (designated as DNA57834-1339) (UNQ465) was disrupted. The gene specific information for these studies is as follows: the mutated mouse gene corresponds to nucleotide reference: NM_{—}025467 Mus musculus RIKEN cDNA 1810036H07 gene (1810036H07Rik); protein reference: Q9CQS6 ACCESSION:Q9CQS6 NID: Mus musculus (Mouse). 1810036H07Rik protein; the human gene sequence reference: NM_{—}182536 Homo sapiens down-regulated in gastric cancer GDDR (GDDR); the human protein sequence corresponds to reference: Q86XP6 ACCESSION:Q86XP6 NID: Homo sapiens (Human). GDDR.

The mouse gene of interest is RIKEN cDNA 1810036H07 gene, ortholog of human GDDR (down-regulated in gastric cancer GDDR).

GDDR is a putative secreted protein, consisting of a signal peptide and a BRICHOS domain (Pfam accession PF04089). Proteins with this domain have been implicated in dementia, respiratory distress, and cancer.

Targeted or gene trap mutations are generated in strain 129SvEv^{Brd}-derived embryonic stem (ES) cells.

The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 13 | 27 | 13 | 53 |
| Expected | 13.25 | 26.5 | 13.25 | 53 |

Chi-Sq.=0.05 Significance=0.9753099 (hom/n)=0.24 Avg. Litter Size=7

### Mutation Information

Mutation Type: Homologous Recombination (standard)
Description: Coding exons 1 through 3 were targeted (NCBI accession NM_{—}025467. 1).
1. Wild-type Expression Panel: Expression of the target gene was detected only in stomach, small intestine, and colon among the 13 adult tissue samples tested by RT-PCR.
2. QC Expression: Disruption of the target gene was confirmed by Southern hybridization analysis.

70.18.1. Phenotypic Analysis (for Disrupted Gene: DNA57834-1339 (UNQ465)

(a) Overall Phenotypic Summary:

Mutation of the gene encoding the ortholog of human down-regulated in gastric cancer GDDR (GDDR) resulted in the observation that (−/−) mice exhibited elevated levels of serum glucose levels when compared with their (+/+) littermates. The (−/−) mutants also showed decreased mean body weight. Gene disruption was confirmed by Southern blot.

(b) Phenotypic Analysis: Metabolism-Blood Chemistry

In the area of metabolism, targets may be identified for the treatment of diabetes. Blood chemistry phenotypic analysis includes blood glucose measurements. The COBAS Integra 400 (mfr: Roche) was used for running blood chemistry tests on the mice. In the area of metabolism, targets may be identified for the treatment of diabetes.

Results:

Both the male and female (−/−) mice exhibited a notably increased mean serum glucose levels when compared with their gender-matched (+/+) littermates and the historical mean.

As summarized above, the (−/−) mice exhibited notably increased mean serum glucose levels suggesting a pre-diabetic condition. Thus, mutant mice deficient in the PRO813 gene can serve as a model for cardiovascular disease including diabetes. PRO813 polypeptides or its encoding gene would be useful in regulating normal blood glucose levels. Thus, PRO813 polypeptides or agonists thereof would be useful in the treatment of such cardiovascular diseases as hypertension, atherosclerosis, heart failure, stroke, various coronary diseases, diabetes and/or obesity.

(c) Bone Metabolism & Body Diagnostics

Tissue Mass & Lean Body Mass Measurements—Dexa

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in total tissue mass (TTM).

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI, i.e., whole body, vertebrae, and both femurs).

Body Measurements (Body Length & Weight):

Body Measurements: A measurement of body length and weight was performed at approximately 16 weeks of age.

Results:

The male (−/−) mice exhibited decreased mean body weight when compared with their gender-matched (+/+) littermates and the historical means.

70.19. Generation and Analysis of Mice Comprising DNA57037-1444 (UNQ469) Gene Disruptions

In these knockout experiments, the gene encoding PRO828 polypeptides (designated as DNA57037-1444) (UNQ469) was disrupted. The gene specific information for these studies is as follows: the mutated mouse gene corresponds to nucleotide reference: NM_{—}024198 ACCESSION:NM_{—}024198 NID:13195625 Mus musculus Mus musculus RIKEN cDNA 3110050F08 gene (3110050F08Rik); protein reference: Q99LJ6 ACCESSION:Q99LJ6 NID: Mus musculus (Mouse). SIMILAR TO RIKEN cDNA 3110050F08 GENE; the human gene sequence reference: NM_{—}015696 ACCESSION:NM_{—}015696 NID:15618996 Homo sapiens Homo sapiens weakly similar to glutathione peroxidase 2 (CL683); the human protein sequence corresponds to reference: Q96SL4 ACCESSION:Q96SL4 NID: Homo sapiens (Human). cDNA FLJ14777 FIS, CLONE NT2RP4000259, WEAKLY SIMILAR TO GLUTATHIONE PEROXIDASE 2 (EC 1.11.1.9).

The mouse gene of interest is Gpx7 (glutathione peroxidase 7), ortholog of human GPX7. Aliases include GPX6, 3110050F08Rik, CL683, FLJ14777, and glutathione peroxidase 6.

GPX7 is a member of the glutathione peroxidase family. The selenoenzyme consists of a signal peptide and a glutathione peroxidase domain (Pfam accession PF00255). Glutathione peroxidases catalyze the glutathione-dependent reduction of hydrogen peroxide and lipid hydroperoxides and play an important role in protecting cells from oxidative damage (Miyamoto et al, Biol Chem 384(4):567-74 (2003)). GPX7 is predicted to be secreted.

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 22 | 46 | 15 | 83 |
| Expected | 20.75 | 41.5 | 20.75 | 83 |

Chi-Sq.=1.92 Significance=0.3828929 (hom/n)=0.21 Avg. Litter Size=8

### Mutation Information

Mutation Type: Homologous Recombination (standard)
Description: Coding exons 2 and 3 were targeted (NCBI accession NM_{—}024198.1).
1. Wild-type Expression Panel: Expression of the target gene was detected in embryonic stem (ES) cells and in all 13 adult tissue samples tested by RT-PCR.
2. QC Expression: Disruption of the target gene was confirmed by Southern hybridization analysis.

70.19.1 Phenotypic Analysis (for Disrupted Gene: DNA57037-1444 (UNQ469)

(a) Overall Phenotypic Summary:

Mutation of the gene encoding the ortholog of human glutathione peroxidase 7 (GPX7) resulted in increased body fat (%) and fat (mass) in (−/−) mice. Gene disruption was confirmed by Southern blot.

(b) Bone Metabolism & Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay.

Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Results:

DEXA: The male (−/−) mice exhibited increased mean percent total body fat and total fat mass when compared with their gender-matched (+/+) littermates and the historical means.

These studies suggest that mutant (−/−) non-human transgenic animals exhibit a negative phenotype that would be associated with obesity. Thus, PRO828 polypeptides or agonists thereof are essential for normal growth and metabolic processes and especially would be important in the prevention and/or treatment of obesity.

70.20. Generation and Analysis of Mice Comprising DNA59619-1464 (UNQ546) Gene Disruptions

In these knockout experiments, the gene encoding PRO1100 polypeptides (designated as DNA59619-1464) (UNQ546) was disrupted. The gene specific information for these studies is as follows: the mutated mouse gene corresponds to nucleotide reference: XM_{—}354640 Mus musculus RIKEN cDNA D430035D22 gene (D430035D22Rik); protein reference: XP_{—}354640 RIKEN cDNA D430035D22 gene [Mus musculus]; the human gene sequence reference: NM_{—}033419 Homo sapiens per1-like domain containing 1 (PERLD1); the human protein sequence corresponds to reference: Q96FM1 ACCESSION: Q96FM1 NID: Homo sapiens (Human). CAB2 protein (AGLA546).

The mouse gene of interest is RIKEN cDNA D430035D22 gene, ortholog of human PERLD1 (per1-like domain containing 1). Aliases include CAB2, PP1498, AGLA546, MGC9753, and CAB2 protein.

PERLD1 is a likely integral membrane protein that is predicted to be located on the plasma membrane. The protein contains a signal peptide and seven transmembrane segments within a Per1-like domain (Katoh and Katoh, Int J Oncol 22(6):1369-74 (2003)). A protein with this domain has been implicated in protein processing in the endoplasmic reticulum (Pfam accession PF04080). PERLD1 is a homolog of yeast COS16, which is required for repair of DNA double-strand breaks (Nezu et al, Jpn J Cancer Res 93(11):1183-6 (2002)). PERLD1 may contribute to the clinical behavior of ERBB2-amplified breast tumors (Kauraniemi et al, Am J Pathol 165 (5): 1979-84 (2003)).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129 SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 16 | 37 | 16 | 69 |
| Expected | 17.25 | 34.5 | 17.25 | 69 |

Chi-Sq.=1.22 Significance=0.5433509 (hom/n)=0.22 Avg. Litter Size=8

### Mutation Information

Mutation Type: Homologous Recombination (standard)
Description: Coding exons 1 through 3 were targeted (NCBI accession AK052486. 1).
1. Wild-type Expression Panel: Expression of the target gene was detected in embryonic stem (ES) cells and in all 13 adult tissue samples tested by RT-PCR, except bone.
2. QC Expression: Disruption of the target gene was confirmed by Southern hybridization analysis.

70.20.1. Phenotypic Analysis (for Disrupted Gene: DNA59619-1464 (UNQ546)

(a) Overall Phenotypic Summary:

Mutation of the gene encoding the ortholog of human per1-like domain containing 1 (PERLD1) resulted in increased total body fat and decreased blood pressure in (−/−) mice. The (−/−) mice also exhibited blood chemistry and behavior abnormalities. The homozygous mutant mice exhibited increased total body fat and serum alkaline phosphatase and phosphorus levels and decreased systolic blood pressure when compared with their gender-matched wild-type littermates and the historical means. In addition, both the male and female homozygous mutants clenched their hind limbs and forelimbs when suspended by the tail, and the male mutants exhibited small faces with short whiskers. The mutant (−/−) mice also exhibited decreased total tissue mass, bone mineral content, BMC/LBM and bone mineral density measurements and increased bone micro CT measurements. Disruption of the target gene was confirmed by Southern hybridization analysis.

(b) Phenotypic Analysis: CNS/Neurology

In the area of neurology, analysis focused herein on identifying in vivo validated targets for the treatment of neurological and psychiatric disorders including depression, generalized anxiety disorders, attention deficit hyperactivity disorder, obsessive compulsive disorder, schizophrenia, cognitive disorders, hyperalgesia and sensory disorders. Neurological disorders include the category defined as “anxiety disorders” which include but are not limited to: mild to moderate anxiety, anxiety disorder due to a general medical condition, anxiety disorder not otherwise specified, generalized anxiety disorder, panic attack, panic disorder with agoraphobia, panic disorder without agoraphobia, posttraumatic stress disorder, social phobia, specific phobia, substance-induced anxiety disorder, acute alcohol withdrawal, obsessive compulsive disorder, agoraphobia, bipolar disorder I or II, bipolar disorder not otherwise specified, cyclothymic disorder, depressive disorder, major depressive disorder, mood disorder, substance-induced mood disorder. In addition, anxiety disorders may apply to personality disorders including but not limited to the following types: paranoid, antisocial, avoidant behavior, borderline personality disorders, dependent, histronic, narcissistic, obsessive-compulsive, schizoid, and schizotypal.

Procedure:

Behavioral screens were performed on a cohort of 4 wild type, 4 heterozygous and 8 homozygous mice. All behavioral tests were done between 12 and 16 weeks of age unless reduced viability necessitates earlier testing. These tests included open field to measure anxiety, activity levels and exploration.

Functional Observational Battery (FOB) Test

The FOB is a series of situations applied to the animal to determine gross sensory and motor deficits. A subset of tests from the Irwin neurological screen that evaluates gross neurological function is used. In general, short-duration, tactile, olfactory, and visual stimuli are applied to the animal to determine their ability to detect and respond normally. These simple tests take approximately 10 minutes and the mouse is returned to its home cage at the end of testing.

Results:

General & Exploratory Activity: The (−/−) mice exhibited decreased rearing activity and decreased hole poke when compared with their (+/+) littermates, suggesting an decreased exploratory response in the mutants.

Open Field Test:

Several targets of known drugs have exhibited phenotypes in the open field test. These include knockouts of the seratonin transporter, the dopamine transporter (Giros et al., Nature. 1996 Feb. 15; 379(6566):606-12), and the GABA receptor (Homanics et al., Proc Natl Acad Sci USA. 1997 Apr. 15; 94(8):4143-8). An automated open-field assay was customized to address changes related to affective state and exploratory patterns related to learning. First, the field (40×40 cm) was selected to be relatively large for a mouse, thus designed to pick up changes in locomotor activity associated with exploration. In addition, there were 4 holes in the floor to allow for nose-poking, an activity specifically related to exploration. Several factors were also designed to heighten the affective state associated with this test. The open-field test is the first experimental procedure in which the mice are tested, and the measurements that were taken were the subjects' first experience with the chamber. In addition, the open-field was brightly lit. All these factors will heighten the natural anxiety associated with novel and open spaces. The pattern and extent of exploratory activity, and especially the center-to-total distance traveled ratio, may then be able to discern changes related to susceptibility to anxiety or depression. A large arena (40 cm×40 cm, VersaMax animal activity monitoring system from AccuScan Instruments) with infrared beams at three different levels was used to record rearing, hole poke, and locomotor activity. The animal was placed in the center and its activity was measured for 20 minutes. Data from this test was analyzed in five, 4-minute intervals. The total distance traveled (cm), vertical movement number (rearing), number of hole pokes, and the center to total distance ratio were recorded.

The propensity for mice to exhibit normal habituation responses to a novel environment is assessed by determining the overall change in their horizontal locomotor activity across the 5 time intervals. This calculated slope of the change in activity over time is determined using normalized, rather than absolute, total distance traveled. The slope is determined from the regression line through the normalized activity at each of the 5 time intervals. Normal habituation is represented by a negative slope value.

Results:

The (−/−) mutant mice exhibited a decreased exploratory response during the open field testing when compared with their gender-matched littermates and the historical means.

A notable difference was observed during open field activity testing. The (−/−) mice exhibited a decreased exploratory response when compared with their gender-matched (+/+) littermates, which is indicative of a decreased anxiety-like response in the mutants. Thus, knockout mice demonstrated a phenotype consistent with depression, generalized anxiety disorders, cognitive disorders, hyperalgesia and sensory disorders and/or bipolar disorders. Thus, PRO1100 polypeptides and agonists thereof would be useful for the treatment or amelioration of the symptoms associated with depressive disorders.

(c) Phenotypic Analysis: Metabolism-Blood Chemistry

In the area of metabolism, targets may be identified for the treatment of metabolic disorders. The COBAS Integra 400 (mfr: Roche) was used for running blood chemistry tests on the mice. In addition to measuring blood glucose levels the following blood chemistry tests are also routinely performed: Alkaline Phosphatase; Alanine Amino-Transferase; Albumin; Bilirubin; Phosphorous; Creatinine; BUN=Blood Urea Nitrogen; Calcium; Uric Acid; Sodium; Potassium; and Chloride.

Results:

Both the male and female (−/−) mice exhibited notably increased mean serum alkaline phosphatase (female p=0.00076; male p=0.017) and phosphorous levels (male p=0.0118; female p=0.256) when compared with their gender-matched (+/+) littermates and the historical means. These results are consistent with the decreased bone-related measurements discussed below.

(d) Diagnostics—Blood Pressure

Description:

Systolic blood pressure is measured via a noninvasive tail-cuff method for four days on the Visitech

BP-2000 Blood Pressure Analysis System. The blood pressure is measured ten times each day for four days. The four days are then averaged to obtain a mouse's conscious systolic blood pressure.

Results: The (−/−) mice exhibited decreased mean systolic blood pressure when compared with their gender-matched (+/+) littermates and the historical mean.

(e) Bone Metabolism & Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Bone MicroCT Analysis:

Procedure: MicroCT was also used to get very sensitive measurements of BMD. One vertebra and 1 femur were taken from a cohort of 4 wild type and 8 homozygous mice. Measurements were taken of lumbar 5 vertebra trabecular bone volume, trabecular thickness, connectivity density and midshaft femur total bone area and cortical thickness. The μCT40 scans provided detailed information on bone mass and architecture. Multiple bones were placed into sample holders and scanned automatically. Instrument software was used to select regions of interest for analysis. Trabecular bone parameters were analyzed in the fifth lumbar vertebrae (LV5) at 16 micrometer resolution and cortical bone parameters were analyzed in the femur midshaft at a resolution of 20 micrometers.

CAT-Scan Protocol:

Mice were injected with a CT contrast agent, Omnipaque 300 (Nycomed Amershan, 300 mg of iodine per ml, 0.25 ml per animal, or 2.50-3.75 g iodine/kg of body weight) intraperitoneally. After resting in the cage for ˜10 minutes, the mouse was then sedated by intraperitoneal injection of Avertin (1.25% 2,2,2,-tribromoethanol, 20 ml/kg body weight). A CAT-scan was performed using a MicroCAT scanner (ImTek, Inc.) with the anesthetized animal lying prone on the test bed. Three dimensional images were reconstructed by the Feldkamp algorithm in a cluster of workstations using an ImTek 3D RECON software.

Results:

General Observations: The male (−/−) mice exhibited short, small faces with decreased whisker length. In addition, the male and female (−/−) mice clench their forelimbs and hind limbs when suspended by the tail. Both male and female homozygotes exhibited a heart rate that was 1-2 standard deviations below the historical means.

DEXA: Both the male and female (−/−) mice exhibited increased mean percent total body fat and total fat mass when compared with their gender-matched (+/+) littermates and the historical means. Ketones were also found in three mutant (−/−) mice indicative of abnormal fat metabolism. In addition, the female (−/−) mice exhibited decreased mean total tissue mass, decreased mean bone mineral content, bone mineral content index (BMC/LBM), and mean bone mineral density in total body and vertebrae when compared with their gender-matched (+/+) littermates and the historical means.
Micro-CT: The male (−/−) mice exhibited increased mean femoral mid-shaft cortical thickness when compared with their gender-matched (+/+) littermates and the historical mean.
CAT-Scan: All of the (−/−) mice analyzed (M-112, M-155, and F-157) exhibited increased abdominal fat depots.

Summary:

Aside from the neurological observations described above for the (−/−) mice, the mutant (−/−) mice analyzed by DEXA, bone micro CT analysis and CAT-scan exhibited decreased bone measurements when compared with their (+/+) littermates, suggestive of abnormal bone disorders. However, the mutant (−/−) mice also exhibited an increased mean percentage of body fat and increased fat deposits in the abdomen suggestive of an obesity phenotype. These observations suggest that mutant mice deficient in the gene which encodes PRO1100 polypeptides leads to metabolic disorders associated with accumulation of fat but also abnormal bone measurements reflective of general metabolic disorders which can be associated with obesity. Thus, PRO1100 polypeptides or agonists thereof would be useful in the treatment or prevention of such disorders as obesity or other metabolic diseases. However, the negative bone phenotype would also suggest that PRO1100 polypeptides or agonists thereof would be useful for maintaining bone homeostasis. In addition, PRO1100 polypeptides would be useful in bone healing or for the treatment of arthritis or osteoporosis, whereas antagonists (or inhibitors of PRO1100 polypeptides or its encoding gene would lead to abnormal or pathological bone disorders including inflammatory diseases associated with abnormal bone metabolism including arthritis, osteoporosis and osteopenia.

70.21. Generation and Analysis of Mice Comprising DNA57033-1403 (UN0557) Gene Disruptions

In these knockout experiments, the gene encoding PRO1114 polypeptides (designated as DNA57033-1403) (UNQ557) was disrupted. The gene specific information for these studies is as follows: the mutated mouse gene corresponds to nucleotide reference: XM_{—}135077 Mus musculus similar to IL20R-beta (LOC213208); protein reference: XP_{—}135077 similar to IL20R-beta [Mus musculus]; the human gene sequence reference: NM_{—}144717 Homo sapiens hypothetical protein MGC34923 (MGC34923); the human protein sequence corresponds to reference: Q6UXL0 ACCESSION:Q6UXL0 NID: Homo sapiens (Human). IL20R-beta.

The mouse gene of interest is expressed sequence AV228068, ortholog of human hypothetical protein MGC34923. Aliases include Gm186 and “similar to IL20R-beta.”

Gm186 is a putative type I plasma membrane protein that likely functions as a receptor or cell adhesion molecule. The protein contains a signal peptide, a fibronectin type III domain (Pfam accession PF00041), a transmembrane segment, and a short cytoplasmic C terminus.

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 16 | 43 | 19 | 78 |
| Expected | 19.5 | 39 | 19.5 | 78 |

Chi-Sq.=2.78 Significance=0.24907531 (hom/n)=0.23 Avg. Litter Size=5

### Mutation Information

Mutation Type: Homologous Recombination (standard)
Description: Coding exon 1 was targeted (NCBI accession XM_{—}13 5077.2).
1. Wild-type Expression Panel: Expression of the target gene was detected in embryonic stem (ES) cells and in all 13 adult tissue samples tested by RT-PCR, except spleen, kidney, liver, and bone.
2. QC Expression: Disruption of the target gene was confirmed by Southern hybridization analysis.

70.21.1. Phenotypic Analysis (for Disrupted Gene: DNA57033-1403 (UNQ557)

(a) Overall Phenotypic Summary:

Mutation of the gene encoding the ortholog of a human hypothetical protein (MGC34923) resulted in the (−/−) mice exhibiting a decreased bone mineral content/lean body mass ratio (BMC/LBM) and decreased total tissue mass. Gene disruption was confirmed by Southern blot.

(b) Bone Metabolism & Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Results:

The male (−/−) mice showed a decreased BMC/LBM index and a decreased total tissue mass when compared with their gender-matched wildtype littermates.

The (−/−) mice analyzed by DEXA exhibited decreased body mass measurements and decreased bone mineral content when compared with their (+/+) littermates, suggestive of abnormal bone disorders. The (−/−) mice exhibited a negative bone phenotype with abnormal and decreased bone measurements reflective of bone metabolic disorders. The negative bone phenotype indicates that PRO1114 polypeptides or agonists thereof would be useful for maintaining bone homeostasis. In addition, PRO1114 polypeptides would be useful in bone healing or for the treatment of arthritis or osteoporosis, whereas antagonists (or inhibitors) of PRO1114 polypeptides or its encoding gene would lead to abnormal or pathological bone disorders including inflammatory diseases associated with abnormal bone metabolism including arthritis, osteoporosis and osteopenia.

70.22. Generation and Analysis of Mice Comprising DNA56868-1478 (UN0558) Gene Disruptions

In these knockout experiments, the gene encoding PRO1115 polypeptides (designated as DNA56868-1478) (UNQ558) was disrupted. The gene specific information for these studies is as follows: the mutated mouse gene corresponds to nucleotide reference: NM_{—}145394 ACCESSION:NM_{—}145394 NID:21703787 Mus musculus Mus musculus similar to choline transporter-like protein (LOC213603); protein reference: Q921V7 ACCESSION:Q921V7 NID: Mus musculus (Mouse). SIMILAR TO TRANSPORTER-LIKE PROTEIN; the human gene sequence reference: NM_{—}152369 Homo sapiens hypothetical protein MGC45474 (MGC45474); the human protein sequence corresponds to reference: Q7Z6C5 ACCESSION:Q7Z6C5 NID: Homo sapiens (Human). Hypothetical protein.

The mouse gene of interest is “cDNA sequence BC010552”, ortholog of human “hypothetical protein MGC45474”. Aliases include MGC18084 and MGC38127.

Hypothetical protein MGC45474 is a likely transporter (KOG1362, choline transporter-like protein [Lipid transport and metabolism]) located in the plasma membrane, consisting of at least seven transmembrane segments and a DUF580 domain. DUF580 domain-containing proteins constitute a unique family ofuncharacterized proteins (Pfam accession PF04515).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 26 | 37 | 18 | 81 |
| Expected | 20.25 | 40.5 | 20.25 | 81 |

Chi-Sq.=7.02 Significance=0.0298969 15 (hom/n)=0.18 Avg. Litter Size=5

### Mutation Information

Mutation Type: Homologous Recombination (standard)
Description: Coding exons 1 and 2 were targeted (NCBI accession NM_{—}145394.1).
1. Wild-type Expression Panel: Expression of the target gene was detected in embryonic stem (ES) cells and in all 13 adult tissue samples tested by RT-PCR, except bone.
2. QC Expression: Disruption of the target gene was confirmed by Southern hybridization analysis.

70.22.1. Phenotypic Analysis (for Disrupted Gene: DNA56868-1478 (UNQ558)

(a) Overall Phenotypic Summary:

Mutation of the gene encoding the ortholog of a human hypothetical protein (MGC45474) resulted in an increased mean femoral mid-shaft cross-sectional area and cortical thickness when compared to the wildtype littermates. Gene disruption was confirmed by Southern blot.

(b) Bone Metabolism & Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Bone MicroCT Analysis:

Procedure: MicroCT was also used to get very sensitive measurements of BMD. One vertebra and 1 femur were taken from a cohort of 4 wild type and 8 homozygous mice. Measurements were taken of lumbar 5 vertebra trabecular bone volume, trabecular thickness, connectivity density and midshaft femur total bone area and cortical thickness. The μCT40 scans provided detailed information on bone mass and architecture. Multiple bones were placed into sample holders and scanned automatically. Instrument software was used to select regions of interest for analysis. Trabecular bone parameters were analyzed in the fifth lumbar vertebrae (LV5) at 16 micrometer resolution and cortical bone parameters were analyzed in the femur midshaft at a resolution of 20 micrometers.

Results:

Micro-CT: The male (−/−) mice exhibited increased mean femoral mid-shaft cross-sectional area and cortical thickness when compared with their gender-matched (+/+) littermates and the historical mean.

In summary, the (−/−) mice exhibited increased femoral mid-shaft cross-sectional area and cortical thickness when compared with their gender-matched (+/+) littermates. These results indicate that the knockout mutant phenotype is associated with such bone abnormalities as osteopetrosis. Osteopetrosis is a condition characterized by abnormal thickening and hardening of bone and abnormal fragility of the bones. As such, PROMS polypeptides or agonists thereof would be beneficial for the treatment of osteopetrosis or other osteo-related diseases. On the other hand, inhibitors or antagonists of PROMS polypeptides would be useful in bone healing.

70.23. Generation and Analysis of Mice Comprising DNA60615-1483 (UNQ564) Gene Disruptions

In these knockout experiments, the gene encoding PRO1126 polypeptides (designated as DNA60615-1483) (UNQ564) was disrupted. The gene specific information for these studies is as follows: the mutated mouse gene corresponds to nucleotide reference: NM_{—}172907 Mus musculus cDNA sequence BC047207 (BC047207); protein reference: Q8BSH2 ACCESSION: Q8BSH2 NID: Mus musculus (Mouse). Mus musculus 12 days embryo male wolffian duct includes surrounding region cDNA, RIKEN full-length enriched library, clone:6720478C22 product:hypothetical protein, full insert sequence (Hypothetical protein BC047207); the human gene sequence reference: NM_{—}198474 Homo sapiens olfactomedin-like 1 (OLFML1); the human protein sequence corresponds to reference: Q6UWY5 ACCESSION:Q6UWY5 NID: Homo sapiens (Human). MVAL564.

The mouse gene of interest is Olfml1 (olfactomedin-like 1), ortholog of human OLFML1. Aliases include MVAL564, MGC56882, 6720478C22, UNQ564, and cDNA sequence BC047207.

OLFML1 is a putative secreted protein that may function as an extracellular matrix protein. OLFM1 consists of a signal peptide and an olfactomedin-like domain (Pfam accession PF02191). Proteins with similar domain organization include olfactomedin and myocilin. Olfactomedin is an extracellular matrix protein specific to olfactory neuroepithelium (Yokoe and Anholt, Proc Natl Acad Sci USA 90(10):4655-9 (1993)).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 23 | 49 | 25 | 97 |
| Expected | 24.25 | 48.5 | 24.25 | 97 |

Chi-Sq.=1.35 Significance=0.5091564 (hom/n)=0.25 Avg. Litter Size=10

### Mutation Information

Mutation Type: Homologous Recombination (standard)
Description: Coding exons 1 and 2 were targeted (NCBI accession NM_{—}172907.2).
1. Wild-type Expression Panel: Expression of the target gene was detected in embryonic stem (ES) cells and in all 13 adult tissue samples tested by RT-PCR, except lung, skeletal muscle, and bone.
2. QC Expression: Disruption of the target gene was confirmed by Southern hybridization analysis.

70.23.1. Phenotypic Analysis (for Disrupted Gene: DNA60615-1483 (UNQ564)

(a) Overall Phenotypic Summary:

Mutation of the gene encoding the ortholog of human olfactomedin-like 1 (OLFML1) resulted in increased mean serum IgM levels in the (−/−) mice. In addition, the homozygous mice exhibited increased mean serum cholesterol levels. Gene disruption was confirmed by Southern blot.

(b) Phenotypic Analysis: Cardiology

In the area of cardiovascular biology, targets were identified herein for the treatment of hypertension, atherosclerosis, heart failure, stroke, various coronary artery diseases, dyslipidemias such as high cholesterol (hypercholesterolemia), diabetes and/or obesity. The phenotypic tests included the measurement of serum cholesterol.

Blood Lipids

Procedure: A cohort of 4 wild type, 4 heterozygous and 8 homozygous mice were tested in this assay. High cholesterol levels are recognized risk factors in the development of cardiovascular disease and/or diabetes. Measuring blood lipids facilitates the finding of biological switches that regulate blood lipid levels Inhibition of factors which elevate blood lipid levels may be useful for reducing the risk for cardiovascular disease. In these blood chemistry tests, cholesterol measurements were recorded using the COBAS Integra 400 (mfr: Roche).

Results:

The male (−/−) mice exhibited an increased mean serum cholesterol level when compared with their gender-matched (+/+) littermates and the historical mean.

As summarized above, the (−/−) mice exhibited notably increased cholesterol levels when compared with their gender-matched (+/+) littermates and the historical means. Thus, mutant mice deficient in the PRO1126 gene can serve as a model for cardiovascular disease. PRO1126 polypeptides or its encoding gene would be useful in regulating blood lipids. Thus, PRO1126 polypeptides or agonists thereof would be useful in the treatment of such cardiovascular diseases as hypertension, atherosclerosis, heart failure, stroke, various coronary diseases, hypercholesterolemia, diabetes and/or obesity.

(c) Immunology Phenotypic Analysis

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

T lymphocytes (T cells) are an important component of a mammalian immune response. T cells recognize antigens which are associated with a self-molecule encoded by genes within the major histocompatibility complex (MHC). The antigen may be displayed together with MHC molecules on the surface of antigen presenting cells, virus infected cells, cancer cells, grafts, etc. The T cell system eliminates these altered cells which pose a health threat to the host mammal. T cells include helper T cells and cytotoxic T cells. Helper T cells proliferate extensively following recognition of an antigen-MHC complex on an antigen presenting cell. Helper T cells also secrete a variety of cytokines, i.e., lymphokines, which play a central role in the activation of B cells, cytotoxic T cells and a variety of other cells which participate in the immune response.

In many immune responses, inflammatory cells infiltrate the site of injury or infection. The migrating cells may be neutrophilic, eosinophilic, monocytic or lymphocytic as can be determined by histologic examination of the affected tissues. Current Protocols in Immunology, ed. John E. Coligan, 1994, John Wiley & Sons, Inc.

Many immune related diseases are known and have been extensively studied. Such diseases include immune-mediated inflammatory diseases (such as rheumatoid arthritis, immune mediated renal disease, hepatobiliary diseases, inflammatory bowel disease (IBD), psoriasis, and asthma), non-immune-mediated inflammatory diseases, infectious diseases, immunodeficiency diseases, neoplasia, and graft rejection, etc. In the area of immunology, targets were identified for the treatment of inflammation and inflammatory disorders.

In the area of immunology, targets have been identified herein for the treatment of inflammation and inflammatory disorders. Immune related diseases, in one instance, could be treated by suppressing the immune response. Using neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

The following test was performed:

Serum Immunoglobulin Isotyping Assay:

The Serum Immunoglobulin Isotyping Assay is performed using a Cytometric Bead Array (CBA) kit. This assay is used to rapidly identify the heavy and light chain isotypes of a mouse monoclonal antibody in a single sample. The values expressed are “relative fluorescence units” and are based on the detection of kappa light chains. Any value <6 is not significant.

Results:

The (−/−) mice exhibited an increased mean serum IgM level when compared with that of their (+/+) littermates and the historical medians.

Mutant (−/−) mice exhibited elevation of IgM serum immunoglobulins compared to their gender-matched (+/+) littermates. IgM immunoglobulins are the first to be produced in a humoral immune response for neutralization of bacterial toxins and are particularly important in activating the complement system. The observed phenotype suggests that the PRO1126 polypeptide is a negative regulator of inflammatory responses.

These immunological abnormalities suggest that inhibitors (antagonists) of PRO1126 polypeptides would be useful in stimulating the immune system (such as T cell proliferation) and would find utility in the cases wherein this effect would be beneficial to the individual such as in the case of leukemia, and other types of cancer, and in immunocompromised patients, such as AIDS sufferers. Accordingly, PRO1126 polypeptides or agonists thereof would be useful in inhibiting the immune response and would be useful candidates for suppressing harmful immune responses, e.g. in the case of graft rejection or graft-versus-host diseases.

70.24. Generation and Analysis of Mice Comprising DNA53913-1490 (UNQ571) Gene Disruptions

In these knockout experiments, the gene encoding PRO1133 polypeptides (designated as DNA53913-1133) (UNQ571) was disrupted. The gene specific information for these studies is as follows: the mutated mouse gene corresponds to nucleotide reference: NM_{—}030699 Mus musculus netrin G1 (Ntng 1); protein reference: Q9ESR3ACCESSION:Q9ESR3NID: Mus musculus (Mouse). NETRIN-G1A; the human gene sequence reference: BC030220 Homo sapiens netrin G1, mRNA (cDNA clone MGC:34337 IMAGE:5206594), complete cds; the human protein sequence corresponds to reference: Q9Y212 Netrin G1 precursor (Laminet-1) (UNQ571/PRO1133).

The mouse gene of interest is Ntng1 (netrin G1), ortholog of human NTNG1. Aliases include Lmnt1, netrin-G1, A930010C08Rik, KIAA0976, and laminet 1.

NTNG1 is a glycosylphosphatidylinositol-anchored extracellular membrane protein that functions as a receptor for netrin G1 ligand (NGL1). NTNG1 is expressed primarily in axons and dendrites of neurons in various brain regions but is also expressed in lung, kidney, testis, and ovary. In brain, NTNG1 and its ligand are involved in guidance and outgrowth of axons and dendrites (Lin et al, Nat Neurosci 6(12):1270-6 (2003); Nakashiba et al, Mech Dev 111(1-2):47-60 (2002); Yin et al, Mol Cell Neurosci 19(3):344-58 (2002); Nakashiba et al, J Neurosci 20 (17):6540-50 (2000)).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 25 | 49 | 17 | 91 |
| Expected | 22.75 | 45.5 | 22.75 | 91 |

Chi-Sq.=2.12 Significance=0.34645584 (hom/n)=0.21 Avg. Litter Size=9

### Mutation Information

Mutation Type: Homologous Recombination (standard)
Description: Coding exons 1 and 2 were targeted (NCBI accession NM_{—}030699.1).
1. Wild-type Expression Panel: Expression of the target gene was detected in brain, spinal cord, eye, spleen, kidney, heart, and adipose among the 13 adult tissue samples tested by RT-PCR.
2. QC Expression: Disruption of the target gene was confirmed by Southern hybridization analysis.

70.24.1. Phenotypic Analysis (for Disrupted Gene: DNA53913-1490 (UNQ571)

(a) Overall Phenotypic Summary:

Mutation of the gene encoding the ortholog of human netrin G1 (NTNG1) resulted in neurological abnormalities in (−/−) mice. The homozygous mutant mice exhibited an increased exploratory response marked by increased rearing when compared with their wild-type littermates. In addition, the female mutants exhibited an abnormal sleep/wake cycle during home-cage activity testing (enhanced circadian rhythm) and almost complete absence of mobility in the tail suspension assay. The (−/−) mice also showed a decreased mean serum IgG3 level.

Disruption of the target gene was confirmed by Southern hybridization analysis.

(b) Phenotypic Analysis: CNS/Neurology

In the area of neurology, analysis focused herein on identifying in vivo validated targets for the treatment of neurological and psychiatric disorders including depression, generalized anxiety disorders, attention deficit hyperactivity disorder, obsessive compulsive disorder, schizophrenia, cognitive disorders, hyperalgesia and sensory disorders. Neurological disorders include the category defined as “anxiety disorders” which include but are not limited to: mild to moderate anxiety, anxiety disorder due to a general medical condition, anxiety disorder not otherwise specified, generalized anxiety disorder, panic attack, panic disorder with agoraphobia, panic disorder without agoraphobia, posttraumatic stress disorder, social phobia, specific phobia, substance-induced anxiety disorder, acute alcohol withdrawal, obsessive compulsive disorder, agoraphobia, bipolar disorder I or II, bipolar disorder not otherwise specified, cyclothymic disorder, depressive disorder, major depressive disorder, mood disorder, substance-induced mood disorder. In addition, anxiety disorders may apply to personality disorders including but not limited to the following types: paranoid, antisocial, avoidant behavior, borderline personality disorders, dependent, histronic, narcissistic, obsessive-compulsive, schizoid, and schizotypal.

Procedure:

Behavioral screens were performed on a cohort of 4 wild type, 4 heterozygous and 8 homozygous mice. All behavioral tests were done between 12 and 16 weeks of age unless reduced viability necessitates earlier testing. These tests included open field to measure anxiety, activity levels and exploration.

Functional Observational Battery (FOB) Test

The FOB is a series of situations applied to the animal to determine gross sensory and motor deficits. A subset of tests from the Irwin neurological screen that evaluates gross neurological function is used. In general, short-duration, tactile, olfactory, and visual stimuli are applied to the animal to determine their ability to detect and respond normally. These simple tests take approximately 10 minutes and the mouse is returned to its home cage at the end of testing.

Results:

General & Exploratory Activity: The (−/−) mice exhibited increased rearing activity when compared with their (+/+) littermates, suggesting an increased exploratory response in the mutants.

The (−/−) mice demonstrated an increased exploratory response which could be associated with bipolar disorders; sensory disorders; obsessive-compulsive disorders, schizophrenia or a paranoid personality. Thus, PRO571 polypeptides or agonists thereof may play a role in the treatment of such neurological disorders.

Functional Observational Battery (FOB) Test—Tail Suspension Testing:

The FOB is a series of situations applied to the animal to determine gross sensory and motor deficits. A subset of tests from the Irwin neurological screen that evaluates gross neurological function is used. In general, short-duration, tactile, olfactory, and visual stimuli are applied to the animal to determine their ability to detect and respond normally. These simple tests take approximately 10 minutes and the mouse is returned to its home cage at the end of testing.

Tail Suspension Testing:

The tail suspension test is a procedure that has been developed as a model for depressive-like behavior in rodents. In this particular setup, a mouse is suspended by its tail for 6 minutes, and in response the mouse will struggle to escape from this position. After a certain period of time the struggling of the mouse decreases and this is interpreted as a type of learned helplessness paradigm. Animals with invalid data (i.e. climbed their tail during the testing period) are excluded from analysis.

Results:

Most (−/−) mice showed an almost complete absence of mobility in the tail suspension testing which is indicative of an increased depressive-response. Thus, knockout mice demonstrated a phenotype consistent with depression, generalized anxiety disorders, cognitive disorders, hyperalgesia and sensory disorders and/or bipolar disorders. Thus, PRO1133 polypeptides and agonists thereof would be useful for the treatment or amelioration of the symptoms associated with depressive disorders.

Circadian Test Description:

Female mice are individually housed at 4 pm on the first day of testing in 48.2 cm×26.5 cm home cages and administered food and water ad libitum. Animals are exposed to a 12-hour light/dark cycle with lights turning on at 7 am and turning off at 7 pm. The system software records the number of beam interruptions caused by the animal's movements, with beam breaks automatically divided into ambulations. Activity is recorded in 60, one-hour intervals during the three-day test. Data generated are displayed by median activity levels recorded for each hour (circadian rhythm) and median total activity during each light/dark cycle (locomotor activity) over the three-day testing period.

Results:

The female (−/−) mice exhibited increased ambulatory counts during the 12-hour habituation period of home-cage activity testing when compared with their gender-matched (+/+) littermates and the historical mean. The female (−/−) mice also exhibited decreased median light-to-dark and light-to-total activity ratios, suggesting an abnormal sleep/wake cycle during the last 24 hours of testing. These results demonstrate an enhanced circadian rhythm.

(c) Immunology Phenotypic Analysis

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

T lymphocytes (T cells) are an important component of a mammalian immune response. T cells recognize antigens which are associated with a self-molecule encoded by genes within the major histocompatibility complex (MHC). The antigen may be displayed together with MHC molecules on the surface of antigen presenting cells, virus infected cells, cancer cells, grafts, etc. The T cell system eliminates these altered cells which pose a health threat to the host mammal. T cells include helper T cells and cytotoxic T cells. Helper T cells proliferate extensively following recognition of an antigen-MHC complex on an antigen presenting cell. Helper T cells also secrete a variety of cytokines, i.e., lymphokines, which play a central role in the activation of B cells, cytotoxic T cells and a variety of other cells which participate in the immune response.

In many immune responses, inflammatory cells infiltrate the site of injury or infection. The migrating cells may be neutrophilic, eosinophilic, monocytic or lymphocytic as can be determined by histologic examination of the affected tissues. Current Protocols in Immunology, ed. John E. Coligan, 1994, John Wiley & Sons, Inc.

Many immune related diseases are known and have been extensively studied. Such diseases include immune-mediated inflammatory diseases (such as rheumatoid arthritis, immune mediated renal disease, hepatobiliary diseases, inflammatory bowel disease (IBD), psoriasis, and asthma), non-immune-mediated inflammatory diseases, infectious diseases, immunodeficiency diseases, neoplasia, and graft rejection, etc. In the area of immunology, targets were identified for the treatment of inflammation and inflammatory disorders.

In the area of immunology, targets have been identified herein for the treatment of inflammation and inflammatory disorders. Immune related diseases, in one instance, could be treated by suppressing the immune response. Using neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

The following test was performed:

Serum Immunoglobulin Isotyping Assay:

The Serum Immunoglobulin Isotyping Assay is performed using a Cytometric Bead Array (CBA) kit. This assay is used to rapidly identify the heavy and light chain isotypes of a mouse monoclonal antibody in a single sample. The values expressed are “relative fluorescence units” and are based on the detection of kappa light chains. Any value <6 is not significant.

Results:

The (−/−) mice exhibited decreased mean serum IgG3 compared to their gender-matched littermate controls.

The serum immunoglobulin isotyping assay revealed that homozygous mice exhibited decreased serum IgG3 levels. Thus, homozygotes showed an abnormally low serum immunoglobulins compared with the (+/+) littermates. Thus, the gene encoding PRO1133 is essential for making immunoglobulins (or gamma globulins). IgG3 immunoglobulins have neutralization effects and to a lesser extent are important for activation of the complement system. These immunological abnormalities suggest that PRO1133 polypeptides or agonists thereof would stimulate the immune system (such as T cell proliferation) and would find utility in the cases wherein this effect would be beneficial to the individual such as in the case of leukemia, and other types of cancer, and in immunocompromised patients, such as AIDS sufferers. Accordingly, inhibitors (antagonists) of PRO1133 polypeptides would inhibit the immune response and would be useful candidates for suppressing harmful immune responses, e.g. in the case of graft rejection or graft-versus-host diseases.

70.25. Generation and Analysis of Mice Comprising DNA59846-1503 (UNQ584) Gene Disruptions

In these knockout experiments, the gene encoding PRO1154 polypeptides (designated as DNA59846-1503) (UNQ584) was disrupted. The gene specific information for these studies is as follows: the mutated mouse gene corresponds to nucleotide reference: NM_{—}030711 Mus musculus type 1 tumor necrosis factor receptor shedding aminopeptidase regulator (Artsl); protein reference: Q9EQH2 ACCESSION:Q9EQH2 NID: Mus musculus (Mouse). ADIPOCYTE-DERIVED LEUCINE AMINOPEPTIDASE PRECURSOR (EC 3.4.11.-) (A-LAP) (ARTS-1) (AMINOPEPTIDASE PILS) (PUROMYCIN-INSENSITIVE LEUCYL-SPECIFIC AMINOPEPTIDASE) (PILS-AP) (VEGF INDUCED AMINOPEPTIDASE); the human gene sequence reference: NM_{—}016442 ACCESSION:NM_{—}016442 NID:20149636 Homo sapiens Homo sapiens type 1 tumor necrosis factor receptor shedding aminopeptidase regulator (ARTS-1); the human protein sequence corresponds to reference: Q6UWY6 ACCESSION:Q6UWY6 NID: Homo sapiens (Human). ARTS-1.

The mouse gene of interest is Arts1 (type 1 tumor necrosis factor receptor shedding aminopeptidase regulator), ortholog of human ARTS-1. Aliases include ERAAP, PILSA, PILSAP, ALAP, A-LAP, ARTS1, ERAP1, APPILS, KIAA0525, adipocyte-derived leucine aminopeptidase, and aminopeptidase PILS.

ARTS-1 is a widely expressed zinc metallopeptidase that catalyzes the release of N-terminal amino acids, with a preference for leucine. This protease has broad substrate specificity and requires zinc for catalysis. ARTS-1 is a type II membrane protein that is located in the endoplasmic reticulum or on the extracellular surface of the plasma membrane (Serwold et al, Nature 419(6906):480-3 (2002); Cui et al, J Clin Invest 110(4):515-26 (2002)). Moreover, this protease has been reported to be secreted (Hattori et al, J Biochem (Tokyo) 128(5): 755-62 (2000)).

ARTS-1 is involved in ectodomain shedding of cytokine receptors, thereby regulating cytokine bio activity (Cui et al, J Biol Chem 278(31):28677-85 (2003a); Cui et al, J Immunol 171(12):6814-9 (2003b); Cui et al, J Clin Invest 110(4):515-26 (2002)). This protease also participates in antigen processing, enabling major histocompatibility complex (MHC) class I molecules to bind and display antigen peptides on the cell surface (York et al, Nat Immunol 3(12):1177-84 (2002); Serwold et al, Nature 419(6906):480-3 (2002)). ARTS-1 may play a role in blood pressure regulation by inactivating angiotensin II or by generating bradykinin in the kidney (Yamamoto et al, Hum Mutat 19(3):251-7 (2002); Hattori et al, J. Biochem (Tokyo) 128(5):755-62 (2000)). ARTS-1 expressed in vascular endothelial cells is induced by vascular endothelial growth factor (VEGF) and is likely to play a role in postnatal angiogenesis (Miyashita et al, Blood 99(9):3241-9 (2002)).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 19 | 43 | 20 | 82 |
| Expected | 20.5 | 41 | 20.5 | 82 |

Chi-Sq.=1.46 Significance=0.48190898 (hom/n)=0.22 Avg. Litter Size=5

### Mutation Information

Mutation Type: Homologous Recombination (standard)
Description: Coding exon i was targeted (NCBI accession AB047552. 1).
1. Wild-type Expression Panel: Expression of the target gene was detected in embryonic stem (ES) cells and, among the 13 adult tissue samples tested by RT-PCR, in liver, heart, and adipose.
2. QC Expression: Disruption of the target gene was confirmed by Southern hybridization analysis.

70.25.1. Phenotypic Analysis (for Disrupted Gene: DNA59846-1503 (UNQ584)

(a) Overall Phenotypic Summary:

Mutation of the gene encoding the ortholog of human type 1 tumor necrosis factor receptor shedding aminopeptidase regulator (ARTS-1) resulted in the observation that (−/−) mice exhibit an increased mean femoral mid-shaft cortical thickness when compared with their gender-matched (+/+) littermates and the historical means. Also, increased blood pressure was observed especially in female (−/−) mice. Gene disruption was confirmed by Southern blot.

(b) Diagnostics—Blood Pressure

Description:

Systolic blood pressure is measured via a noninvasive tail-cuff method for four days on the Visitech BP-2000 Blood Pressure Analysis System. The blood pressure is measured ten times each day for four days. The four days are then averaged to obtain a mouse's conscious systolic blood pressure.

Results:

The male and female (−/−) mice exhibited increased mean systolic blood pressure (more notable in females) when compared with their gender-matched (+/+) littermates. This observation is indicative of hypertension.

(c) Bone Metabolism & Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Bone MicroCT Analysis:

Procedure: MicroCT was also used to get very sensitive measurements of BMD. One vertebra and 1 femur were taken from a cohort of 4 wild type and 8 homozygous mice. Measurements were taken of lumbar 5 vertebra trabecular bone volume, trabecular thickness, connectivity density and midshaft femur total bone area and cortical thickness. The μCT40 scans provided detailed information on bone mass and architecture. Multiple bones were placed into sample holders and scanned automatically. Instrument software was used to select regions of interest for analysis. Trabecular bone parameters were analyzed in the fifth lumbar vertebrae (LV5) at 16 micrometer resolution and cortical bone parameters were analyzed in the femur midshaft at a resolution of 20 micrometers.

Results:

Micro-CT: The male (−/−) mice exhibited increased mean femoral mid-shaft cortical thickness when compared with their gender-matched (+/+) littermates and the historical mean.

In summary, the (−/−) mice exhibited increased femoral mid-shaft cross-sectional thickness when compared with their gender-matched (+/+) littermates. These results indicate that the knockout mutant phenotype is associated with such bone abnormalities as osteopetrosis. Osteopetrosis is a condition characterized by abnormal thickening and hardening of bone and abnormal fragility of the bones. As such, PRO1154 polypeptides or agonists thereof would be beneficial for the treatment of osteopetrosis or other osteo-related diseases. On the other hand, inhibitors or antagonists of PRO1154 polypeptides would be useful in bone healing.

70.26. Generation and Analysis of Mice Comprising DNA62881-1515 (UNQ599) Gene Disruptions

In these knockout experiments, the gene encoding PRO1185 polypeptides (designated as DNA62881-1515) (UNQ599) was disrupted. The gene specific information for these studies is as follows: the mutated mouse gene corresponds to nucleotide reference: BC051501 Mus musculus mRNA similar to hepatocellular carcinoma-associated gene TD26 (cDNA clone IMAGE:1313868); protein reference: Q80WX2 ACCESSION:Q80WX2 NID: Mus musculus (Mouse). Similar to hepatocellular carcinoma-associated gene TD26 (Fragment); the human gene sequence reference: NM_{—}018687 Homo sapiens hepato cellular carcinoma-associated gene TD26 (L0055908); the human protein sequence corresponds to reference: Q9NQZ1 ACCESSION: Q9NQZ1 NID: Homo sapiens (Human). Hepatocellular carcinoma associated protein TD26.

The mouse gene of interest is “similar to hepatocellular carcinoma-associated gene TD26” (NCBI accession BC051501), ortholog of human “hepatocellular carcinoma-associated gene TD26.” Aliases include PRO1185 and PVPA599.

The hypothetical protein of about 250 amino acids may be a type II membrane protein, containing a potential signal anchor. The function and cell location of this hypothetical protein is not known.

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 26 | 44 | 25 | 95 |
| Expected | 23.75 | 47.5 | 23.75 | 95 |

Chi-Sq.=1.2 Significance=0.5488116 (hom/n)=0.22 Avg. Litter Size=10

### Mutation Information

Mutation Type: Homologous Recombination (standard)
Description: Coding exons 1 through 4 were targeted (NCBI accession BC024408. 1).
1. Wild-type Expression Panel: Expression of the target gene was detected in embryonic stem (ES) cells and in all 13 adult tissue samples tested by RT-PCR, except bone.
2. QC Expression: Disruption of the target gene was confirmed by Southern hybridization analysis.

70.26.1. Phenotypic Analysis (for Disrupted Gene: DNA62881-1515 (UNQ599)

(a) Overall Phenotypic Summary:

Mutation of the gene encoding the ortholog of human “hepatocellular carcinoma-associated gene TD26” resulted in decreased triglycerides in (−/−) mice. Both the male and female homozygous mutant mice exhibited notably decreased mean serum triglyceride levels when compared with their gender-matched wild-type littermates and the historical means. Disruption of the target gene was confirmed by Southern hybridization analysis.

(b) Phenotypic Analysis: Cardiology

In the area of cardiovascular biology, targets were identified herein for the treatment of hypertension, atherosclerosis, heart failure, stroke, various coronary artery diseases, dyslipidemias such as high cholesterol (hypercholesterolemia) and elevated serum triglycerides (hypertriglyceridemia), diabetes and/or obesity. The phenotypic tests included the measurement of serum cholesterol and triglycerides.

Blood Lipids

Procedure: A cohort of 4 wild type, 4 heterozygous and 8 homozygous mice were tested in this assay. High cholesterol levels and increased triglyceride blood levels are recognized risk factors in the development of cardiovascular disease and/or diabetes. Measuring blood lipids facilitates the finding of biological switches that regulate blood lipid levels Inhibition of factors which elevate blood lipid levels may be useful for reducing the risk for cardiovascular disease. In these blood chemistry tests, cholesterol measurements were recorded using the COBAS Integra 400 (mfr: Roche).

Results:

Both the male and female (−/−) mice exhibited notably decreased mean serum triglyceride levels when compared with their gender-matched (+/+) littermates and the historical mean. Ketones were found in two of the four (+/−) mice and two of eight (−/−) mice.

Thus, mutant mice deficient in the PRO1185 encoding gene can serve as a model for treatment of cardiovascular disease especially those diseases which are associated with dyslipidemia Inhibitors (antagonists) of PRO1185 polypeptides or its encoding gene would be useful in regulating blood lipids and in particular for maintaining normal levels of triglycerides. Thus, antagonists of PRO1185 polypeptides would be useful in the treatment of such cardiovascular diseases as: hypertension, atherosclerosis, hypertriglyceridemia, heart failure, stroke, various coronary artery diseases, and/or obesity or diabetes.

70.27. Generation and Analysis of Mice Comprising DNA57841-1522 (UNQ607) Gene Disruptions

In these knockout experiments, the gene encoding PRO1194 polypeptides (designated as DNA57841-1522) (UNQ607) was disrupted. The gene specific information for these studies is as follows: the mutated mouse gene corresponds to nucleotide reference: NM_{—}181417 Mus musculus cysteine and glycine-rich protein 2 binding protein (Csrp2 bp); protein reference: Q8CIDO ACCESSION:Q8CIDO NID: Mus musculus (Mouse). Similar to CSRP2 binding protein; the human gene sequence reference: NM_{—}020536 Homo sapiens CSRP2 binding protein (CSRP2BP), transcript variant 1; the human protein sequence corresponds to reference: Q9H8E8 ACCESSION:Q9H8E8 NID: Homo sapiens (Human). Cysteine-rich protein 2 binding protein (CSRP2 binding protein) (CRP2BP) (CRP2 binding partner).

The mouse gene of interest is Csrp2 bp (cysteine and glycine-rich protein 2 binding protein), ortholog of human CSRP2BP(CSRP2 binding protein). Aliases include D2Ertd473e, E430020F17, 2510008M08Rik, cysteine-rich protein 2 binding protein, CRP2BP, MGC15388, dJ717M23.1, CRP2 binding partner, and CRP2 binding protein.

CSRP2BP is a nuclear and cytoplasmic protein that binds with cysteine and glycine-rich protein 2 (Weiskirchen and Gressner, Biochem Biophys Res Commun 274(3):655-63 (2000)). The protein contains an acetyltransferase domain, suggesting that CSRP2BP may function as an enzyme. Proteins with this domain include histone acetylases, which catalyze the transfer of acetyl groups from acetyl-CoA to the lysine E-amino groups on histones (Pfam accession PF00538). The mainly nuclear localization of CSRP2BP suggests that it may play a role in regulating gene transcription.

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 17 | 36 | 0 | 53 |
| Expected | 13.25 | 26.5 | 13.25 | 53 |

Chi-Sq.=46.85 Significance=6.708912E-11 (hom/n)=0.0 Avg. Litter Size=5

### Mutation Information

Mutation Type: Retroviral Insertion (OST)
Description: Retroviral insertion occurred in the intron between coding exons 2 and 3 (NCBI accession NM_{—}181417.2).
1. Wild-type Expression Panel: Expression of the target gene was detected in embryonic stem (ES) cells and in all 13 adult tissue samples tested by RT-PCR, except adipose.
2. QC Expression: Due to lethality, transcript expression analysis was not performed. Disruption ofthe target gene was confirmed by Inverse PCR.

70.27.1. Phenotypic Analysis (for Disrupted Gene: DNA57841-1522 (UNQ607)

(a) Overall Phenotypic Summary:

Mutation of the gene encoding the ortholog of human CSRP2 binding protein (CSRP2BP) resulted in lethality of (−/−) mutants. Genetic data indicate that this retroviral insertion resulted in lethality of the homozygous mutants. Due to lethality, transcript expression analysis was not performed. Heterozygous (+/−) mice exhibited an increased mean percentage of B cells in the peripheral blood.

Discussion Related to Embryonic Developmental Abnormality of Lethality:

Embryonic lethality in knockout mice usually results from various serious developmental problems including but not limited to neurodegenerative diseases, angiogenic disorders, inflammatory diseases, or where the gene/protein has an important role in basic cell signaling processes in many cell types. In addition, embryonic lethals are useful as potential cancer models. Likewise, the corresponding heterozygous (+/−) mutant animals are particularly useful when they exhibit a phenotype and/or a pathology report which reveals highly informative clues as to the function of the knocked-out gene. For instance, EPO knockout animals were embryonic lethals, but the pathology reports on the embryos showed a profound lack of RBCs.

(b) Pathology

Microscopic Observations: Not tested due to embryonic lethality. At 12.5 days, 48 embryos were observed: 21 (+/−) embryos, 20 (+/+) embryos, 6 resorption moles, and 1 inconclusive.

Gene Expression: Expression of the neo transcript was not detected in the panel of tissues analyzed by in situ hybridization.

(c) Immunology Phenotypic Analysis

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

T lymphocytes (T cells) are an important component of a mammalian immune response. T cells recognize antigens which are associated with a self-molecule encoded by genes within the major histocompatibility complex (MHC). The antigen may be displayed together with MHC molecules on the surface of antigen presenting cells, virus infected cells, cancer cells, grafts, etc. The T cell system eliminates these altered cells which pose a health threat to the host mammal. T cells include helper T cells and cytotoxic T cells. Helper T cells proliferate extensively following recognition of an antigen-MHC complex on an antigen presenting cell. Helper T cells also secrete a variety of cytokines, i.e., lymphokines, which play a central role in the activation of B cells, cytotoxic T cells and a variety of other cells which participate in the immune response.

In many immune responses, inflammatory cells infiltrate the site of injury or infection. The migrating cells may be neutrophilic, eosinophilic, monocytic or lymphocytic as can be determined by histologic examination of the affected tissues. Current Protocols in Immunology, ed. John E. Coligan, 1994, John Wiley & Sons, Inc.

Many immune related diseases are known and have been extensively studied. Such diseases include immune-mediated inflammatory diseases (such as rheumatoid arthritis, immune mediated renal disease, hepatobiliary diseases, inflammatory bowel disease (IBD), psoriasis, and asthma), non-immune-mediated inflammatory diseases, infectious diseases, immunodeficiency diseases, neoplasia, and graft rejection, etc. In the area of immunology, targets were identified for the treatment of inflammation and inflammatory disorders.

In the area of immunology, targets have been identified herein for the treatment of inflammation and inflammatory disorders. Immune related diseases, in one instance, could be treated by suppressing the immune response. Using neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

The following test was performed:

Flourescence-activated Cell-sorting (FACS) Analysis

Procedure:

FACS analysis of immune cell composition from peripheral blood was performed including CD4, CD8 and T cell receptor to evaluate T lymphocytes, CD19 for B lymphocytes, CD45 as a leukocyte marker and pan NK for natural killer cells. The FACS analysis was carried out on wild type and heterozygous mice and included cells derived from thymus, spleen, bone marrow and lymph node.

In these studies, analyzed cells were isolated from thymus, peripheral blood, spleen, bone marrow and lymph nodes. Flow cytometry was designed to determine the relative proportions of CD4 and CD8 positive T cells, B cells, NK cells and monocytes in the mononuclear cell population. A Becton-Dickinson FACSCalibur 3-laser FACS machine was used to assess immune status. For Phenotypic Assays and Screening, this machine records CD4+/CD8−, CD8+/CD4−, NK, B cell and monocyte numbers in addition to the CD4+/CD8+ratio. The mononuclear cell profile was derived by staining a single sample of lysed peripheral blood from each mouse with a panel of six lineage-specific antibodies: CD45 PerCP, anti-TCRb APC, CD4 PE, CD8 FITC, pan-NK PE, and CD19 FITC. The two FITC and PE labeled antibodies stain mutually exclusive cell types. The samples were analyzed using a Becton Dickinson FACSCalibur flow cytometer with CellQuest software.

Results:

FACS:

The heterozygous (+/−) mice exhibited an increased mean percentage of B cells in the peripheral blood when compared with their (+/+) littermates and the historical mean.

Thus, FACS showed that the heterozygous mutant mice exhibit an increased mean percentage of mature B cells in peripheral blood compared with their wild-type littermates.

70.28. Generation and Analysis of Mice Comprising DNA61755-1554 (UNQ656) Gene Disruptions

In these knockout experiments, the gene encoding PRO1287 polypeptides (designated as DNA61755-1554) (UNQ656) was disrupted. The gene specific information for these studies is as follows: the mutated mouse gene corresponds to nucleotide reference: NM_{—}172753 Mus musculus RIKEN cDNA 4732435N03 gene (4732435N03Rik); protein reference: Q8BJQ9 ACCESSION:Q8BJQ9 NID: Mus musculus (Mouse). Mus musculus 10 days neonate cortex cDNA, RIKEN full-length enriched library, clone:A830097M06 product:hypothetical Nucleotide-diphospho-sugar transferases structure containing protein, full insert sequence; the human gene sequence reference: NM_{—}018371 Homo sapiens chondroitin beta-1,4 N-acetylgalactosaminyltransferase (ChGn); the human protein sequence corresponds to reference: Q8TDX6 ACCESSION:Q8TDX6 NID: Homo sapiens (Human). Chondroitin beta-1,4 N-acetylgalactosaminyltransferase (MMVR656).

The mouse gene of interest is RIKEN cDNA 4732435N03 gene, ortholog of human ChGn (chondroitin beta-1,4 N-acetylgalactosaminyltransferase). Aliases include FLJ11264; CSGalNAcT-1; beta4GalNAcT; and beta-1,4 N-acetylgalactosaminyltransferase.

ChGn is a glycosyltransferase that catalyzes the transfer of N-acetylgalactosamine to glucuronic acid in chondroitin sulfate proteoglycans, a component of extracellular matrix. The enzyme is a type II membrane protein predicted to be located in the endoplasmic reticulum or Golgi apparatus. ChGn is likely to be important for initiation of chondroitin sulfate biosynthesis (Sato et al, J Biol Chem 278(5):3063-71 (2003); Uyama et al, J Biol Chem 278(5):3072-8 (2003); Gotoh et al, J Biol Chem 277(41):38189-96 (2002); Uyama et al, J Biol Chem 277(11):8841-6 (2002); Nadanaka et al, Biochem J 340(Pt2):353-7 (1999)).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 13 | 28 | 21 | 62 |
| Expected | 15.5 | 31 | 15.5 | 62 |

Chi-Sq.=4.04 Significance=0.13265547 (hom/n)=0.25 Avg. Litter Size=8

### Mutation Information

Mutation Type: Homologous Recombination (standard)
Description: Coding exon 2 was targeted (NCBI accession NM_{—}172753.2).
1. Wild-type Expression Panel: Expression of the target gene was detected in embryonic stem (ES) cells and in all 13 adult tissue samples tested by RT-PCR, except skeletal muscle; bone; and stomach, small intestine, and colon.
2. QC Expression: Disruption of the target gene was confirmed by Southern hybridization analysis.

70.28.1. Phenotypic Analysis (for Disrupted Gene: DNA61755-1554 (UNQ656)

(a) Overall Phenotypic Summary:

Mutation of the gene encoding the ortholog of human chondroitin beta1,4 N-acetylgalactosaminyltransferase (ChGn) resulted in a decreased anxiety-related response in male (−/−) mice. Organ weights data showed thymic atrophy in a female (−/−) mouse. The male homozygous mutant mice exhibited a decreased anxiety-like response during open field activity testing when compared with their gender-matched wild-type littermates and the historical mean. The (−/−) mice also exhibited decreased bone-related measurements as well as increased total tissue mass and body fat. Disruption of the target gene was confirmed by Southern hybridization analysis.

(b) Phenotypic Analysis: CNS/Neurology

In the area of neurology, analysis focused herein on identifying in vivo validated targets for the treatment of neurological and psychiatric disorders including depression, generalized anxiety disorders, attention deficit hyperactivity disorder, obsessive compulsive disorder, schizophrenia, cognitive disorders, hyperalgesia and sensory disorders. Neurological disorders include the category defined as “anxiety disorders” which include but are not limited to: mild to moderate anxiety, anxiety disorder due to a general medical condition, anxiety disorder not otherwise specified, generalized anxiety disorder, panic attack, panic disorder with agoraphobia, panic disorder without agoraphobia, posttraumatic stress disorder, social phobia, specific phobia, substance-induced anxiety disorder, acute alcohol withdrawal, obsessive compulsive disorder, agoraphobia, bipolar disorder I or II, bipolar disorder not otherwise specified, cyclothymic disorder, depressive disorder, major depressive disorder, mood disorder, substance-induced mood disorder. In addition, anxiety disorders may apply to personality disorders including but not limited to the following types: paranoid, antisocial, avoidant behavior, borderline personality disorders, dependent, histronic, narcissistic, obsessive-compulsive, schizoid, and schizotypal.

Procedure:

Behavioral screens were performed on a cohort of 4 wild type, 4 heterozygous and 8 homozygous mice. All behavioral tests were done between 12 and 16 weeks of age unless reduced viability necessitates earlier testing.

These tests included open field to measure anxiety, activity levels and exploration.

Open Field Test:

Several targets of known drugs have exhibited phenotypes in the open field test. These include knockouts of the seratonin transporter, the dopamine transporter (Giros et al., Nature. 1996 Feb. 15; 379(6566):606-12), and the GABA receptor (Homanics et al., Proc Natl Acad Sci USA. 1997 Apr. 15; 94(8):4143-8). An automated open-field assay was customized to address changes related to affective state and exploratory patterns related to learning. First, the field (40×40 cm) was selected to be relatively large for a mouse, thus designed to pick up changes in locomotor activity associated with exploration. In addition, there were 4 holes in the floor to allow for nose-poking, an activity specifically related to exploration. Several factors were also designed to heighten the affective state associated with this test. The open-field test is the first experimental procedure in which the mice are tested, and the measurements that were taken were the subjects' first experience with the chamber. In addition, the open-field was brightly lit. All these factors will heighten the natural anxiety associated with novel and open spaces. The pattern and extent of exploratory activity, and especially the center-to-total distance traveled ratio, may then be able to discern changes related to susceptibility to anxiety or depression. A large arena (40 cm×40 cm, VersaMax animal activity monitoring system from AccuScan Instruments) with infrared beams at three different levels was used to record rearing, hole poke, and locomotor activity. The animal was placed in the center and its activity was measured for 20 minutes. Data from this test was analyzed in five, 4-minute intervals. The total distance traveled (cm), vertical movement number (rearing), number of hole pokes, and the center to total distance ratio were recorded.

The propensity for mice to exhibit normal habituation responses to a novel environment is assessed by determining the overall change in their horizontal locomotor activity across the 5 time intervals. This calculated slope of the change in activity over time is determined using normalized, rather than absolute, total distance traveled. The slope is determined from the regression line through the normalized activity at each of the 5 time intervals. Normal habituation is represented by a negative slope value.

Results:

The male (−/−) mice exhibited an increased median sum time-in-center when compared with their gender-matched (+/+) littermates and the historical mean, suggesting a decreased anxiety-like response in the mutants.

A notable difference was observed during open field activity testing. The male (−/−) mice exhibited an increased median sum time in the center area when compared with their gender-matched (+/+) littermates, which is indicative of a decreased anxiety-like response in the mutants. Thus, knockout mice demonstrated a phenotype consistent with depression, generalized anxiety disorders, cognitive disorders, hyperalgesia and sensory disorders and/or bipolar disorders. Thus, PRO1287 polypeptides and agonists thereof would be useful for the treatment or amelioration of the symptoms associated with depressive disorders.

(c) Bone Metabolism & Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Bone MicroCT Analysis:

Procedure: MicroCT was also used to get very sensitive measurements of BMD. One vertebra and 1 femur were taken from a cohort of 4 wild type and 8 homozygous mice. Measurements were taken of lumbar 5 vertebra trabecular bone volume, trabecular thickness, connectivity density and midshaft femur total bone area and cortical thickness. The μCT40 scans provided detailed information on bone mass and architecture. Multiple bones were placed into sample holders and scanned automatically. Instrument software was used to select regions of interest for analysis. Trabecular bone parameters were analyzed in the fifth lumbar vertebrae (LV5) at 16 micrometer resolution and cortical bone parameters were analyzed in the femur midshaft at a resolution of 20 micrometers.

Results:

DEXA: The male (−/−) mice exhibited decreased mean bone mineral content and bone mineral content index when compared with their gender-matched (+/+) littermates and the historical means. In addition, the (−/−) mice showed increased in total tissue mass, fat (%) and fat mass (g) compared with their gender-matched (+/+) littermates and the historical controls.
Micro-CT: The male (−/−) mice exhibited decreased mean femoral midshaft cross-sectional area when compared with their gender-matched (+/+) littermates and the historical mean.

The (−/−) mice analyzed by DEXA and bone micro CT analysis exhibited decreased bone measurements when compared with their (+/+) littermates, suggestive of abnormal bone disorders. However, the mutant (−/−) mice also exhibited an increased mean percentage of body fat suggestive of an obesity phenotype. These observations suggest that mutant mice deficient in the gene which encodes PRO1287 polypeptides leads to metabolic disorders associated with accumulation of fat but also abnormal bone measurements reflective of general metabolic disorders which can be associated with obesity. Thus, PRO1287 polypeptides or agonists thereof would be useful in the treatment or prevention of such disorders as obesity or other metabolic diseases. However, the negative bone phenotype would also suggest that PRO1287 polypeptides or agonists thereof would be useful for maintaining bone homeostasis. In addition, PRO1287 polypeptides would be useful in bone healing or for the treatment of arthritis or osteoporosis, whereas antagonists (or inhibitors) of PRO1287 polypeptides or its encoding gene would lead to abnormal or pathological bone disorders including inflammatory diseases associated with abnormal bone metabolism including arthritis, osteoporosis and osteopenia.

70.29. Generation and Analysis of Mice Comprising DNA59610-1556 (UNQ659) Gene Disruptions

In these knockout experiments, the gene encoding PRO1291 polypeptides (designated as DNA59610-1556) (UNQ659) was disrupted. The gene specific information for these studies is as follows: the mutated mouse gene corresponds to nucleotide reference: NM_{—}178594 Mus musculus cDNA sequence BC032925 (BC032925); protein reference: Q8K091 ACCESSION:Q8K091 NID: Mus musculus (Mouse) Similar to hypothetical protein FLJ22418; the human gene sequence reference: NM_{—}024626 ACCESSION:NM_{—}024626 NID: gi 13375849 ref NM_{—}024626.1 Homo sapiens hypothetical protein FLJ22418 (FLJ22418); the human protein sequence corresponds to reference: Q9H₆B2 ACCESSION:Q9H₆B2 NID: Homo sapiens (Human). Hypothetical protein FLJ22418.

The mouse gene of interest is cDNA sequence BC032925, ortholog of human B7-H4 (immune costimulatory protein B7-H4). Aliases include B7S1, B7-H4, MGC41287, B7X, FLJ22418, and immune costimulatory protein B7-H4.

B7-H4 is a glycosyl phosphatidylinositol (GPI)-anchored extracellular membrane protein that functions as a ligand or costimulatory molecule. The protein is expressed on immune cells, nonlymphoid tissues, and some tumor cell lines. B7-H4 binds with a cognate receptor expressed on the surface of activated T cells, thereby blocking T cell proliferation and cytokine production and negatively regulating cell-mediated immunity in peripheral tissues (Sica et al, Immunity 18(6):849-61 (2003); Prasad et al, Immunity 18(6):863-73 (2003); Zang et al, Proc Natl Acad Sci USA 100(18):10388-92 (2003); Choi et al, J Immunol 171(9):4650-4 (2003)).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 21 | 34 | 19 | 74 |
| Expected | 18.5 | 37 | 18.5 | 74 |

Chi-Sq.=3.51 Significance=0.17290725 (hom/n)=0.22 Avg. Litter Size=9

### Mutation Information

Mutation Type: Homologous Recombination (standard)
Description: Coding exon 1 was targeted (NCBI accession NM_{—}178594.2).
1. Wild-type Expression Panel: Expression of the target gene was detected in embryonic stem (ES) cells and in all 13 adult tissue samples tested by RT-PCR, except bone.
2. QC Expression: Disruption of the target gene was confirmed by Southern hybridization analysis.

70.29.1. Phenotypic Analysis (for Disrupted Gene: DNA59610-1556 (UNQ659)

(a) Overall Phenotypic Summary:

Mutation of the gene encoding the ortholog of human immune costimulatory protein B7-H4 (B7-H4) resulted in an increase in stress-induced hyperthermia in (−/−) mice. In addition, both the heterozygous (+/−) and homozygous (−/−) mice exhibited an increased mean percent total body fat and total fat mass and decreased trabecular bone volume and thickness. Gene disruption was confirmed by Southern blot.

(b) Phenotypic Analysis: CNS/Neurology

In the area of neurology, analysis focused herein on identifying in vivo validated targets for the treatment of neurological and psychiatric disorders including depression, generalized anxiety disorders, attention deficit hyperactivity disorder, obsessive compulsive disorder, schizophrenia, cognitive disorders, hyperalgesia and sensory disorders. Neurological disorders include the category defined as “anxiety disorders” which include but are not limited to: mild to moderate anxiety, anxiety disorder due to a general medical condition, anxiety disorder not otherwise specified, generalized anxiety disorder, panic attack, panic disorder with agoraphobia, panic disorder without agoraphobia, posttraumatic stress disorder, social phobia, specific phobia, substance-induced anxiety disorder, acute alcohol withdrawal, obsessive compulsive disorder, agoraphobia, bipolar disorder I or II, bipolar disorder not otherwise specified, cyclothymic disorder, depressive disorder, major depressive disorder, mood disorder, substance-induced mood disorder. In addition, anxiety disorders may apply to personality disorders including but not limited to the following types: paranoid, antisocial, avoidant behavior, borderline personality disorders, dependent, histronic, narcissistic, obsessive-compulsive, schizoid, and schizotypal.

Procedure:

Behavioral screens were performed on a cohort of 4 wild type, 4 heterozygous and 8 homozygous mice. All behavioral tests were done between 12 and 16 weeks of age unless reduced viability necessitates earlier testing. These tests included open field to measure anxiety, activity levels and exploration.

Functional Observational Battery (FOB) Test—Stress-induced Hyperthermia:

The FOB is a series of situations applied to the animal to determine gross sensory and motor deficits. A subset of tests from the Irwin neurological screen that evaluates gross neurological function is used. In general, short-duration, tactile, olfactory, and visual stimuli are applied to the animal to determine their ability to detect and respond normally. These simple tests take approximately 10 minutes and the mouse is returned to its home cage at the end of testing.

Results:

Anxiety: The male (−/−) mice exhibited increased sensitivity to stress-induced hyperthermia when compared with their gender-matched (+/+) littermates and the historical mean, suggesting an increased anxiety-like response in the mutants. In summary, the functional observation testing revealed a phenotype associated with increased anxiety which could be associated with mild to moderate anxiety, anxiety due to a general medical condition, and/or bipolar disorders; hyperactivity; sensory disorders; obsessive-compulsive disorders, schizophrenia or a paranoid personality. Thus, PRO1291 polypeptides or agonists thereof would be useful in the treatment of such neurological disorders.

(c) Bone Metabolism & Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

### Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Results:

DEXA: The male (+/−) and (−/−) mice exhibited increased mean percent total body fat and total fat mass when compared with their gender-matched (+/+) littermates and the historical means.
MicroCT: The (−/−) knockout mice exhibited decreased trabecular bone volume and thickness when compared to their (+/+) littermates.

The (−/−) mice analyzed by bone micro CT exhibited decreased bone measurements when compared with their (+/+) littermates, suggestive of abnormal bone disorders. However, male mutant (−/−) and (+/−) mice also exhibited an increased mean percentage of body fat suggestive of an obesity phenotype. These observations suggest that mutant mice deficient in the gene which encodes PRO1291 polypeptides leads to metabolic disorders associated with accumulation of fat but also abnormal bone measurements reflective of general metabolic disorders which can be associated with obesity. Thus, PRO1291 polypeptides or agonists thereof would be useful in the treatment or prevention of such disorders as obesity or other metabolic diseases. However, the negative bone phenotype would also suggest that PRO1291 polypeptides or agonists thereof would be useful for maintaining bone homeostasis. In addition, PRO1291 polypeptides would be useful in bone healing or for the treatment of arthritis or osteoporosis, whereas antagonists (or inhibitors) of PRO1291 polypeptides or its encoding gene would lead to abnormal or pathological bone disorders including inflammatory diseases associated with abnormal bone metabolism including arthritis, osteoporosis and osteopenia.

70.30. Generation and Analysis of Mice Comprising DNA60618-1557 (UNQ662) Gene Disruptions

In these knockout experiments, the gene encoding PRO1293 polypeptides (designated as DNA60618-1557) (UNQ662) was disrupted. The gene specific information for these studies is as follows: the mutated mouse gene corresponds to nucleotide reference: NM_{—}024263 ACCESSION:NM_{—}024263 NID:15277326 Mus musculus Mus musculus RIKEN cDNA 1200013A08 gene (1200013A08Rik); protein reference: Q920S7 ACCESSION:Q920S7 NID: Mus musculus (Mouse). ADIPOCYTE-SPECIFIC PROTEIN 3; the human gene sequence reference: NM_{—}032348 ACCESSION:NM032348 NID: 14150144 Homo sapiens Homo sapiens hypothetical protein MGC3047 (MGC3047); the human protein sequence corresponds to reference: Q9BRK3 ACCESSION:Q9BRK3 NID: Homo sapiens (Human). SIMILAR TO RIKEN cDNA 1200013A08 GENE (HYPOTHETICAL 49.1 KDA PROTEIN).

The mouse gene of interest is RIKEN cDNA 1200013A08 gene, ortholog of human hypothetical protein MGC3047. Aliases include limitrin and adipocyte-specific protein 3 (NCBI accession AB040488). Limitrin is a type I plasma membrane protein that likely functions as a cell adhesion molecule or receptor. The protein consists a signal peptide, two immunoglobulin-like domains (SMART accession SM00409), a transmembrane segment, and a short cytoplasmic C terminus. Limitrin is expressed in astroglial endfeet in the perivascular region and under the pia mater. Limitrin is likely to be a component of the blood-brain barrier (Yonezawa et al, Glia 44(3):190-204 (2003)).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 17 | 36 | 15 | 68 |
| Expected | 17 | 34 | 17 | 68 |

Chi-Sq.=0.68 Significance=0.7117703 (hom/n)=0.27 Avg. Litter Size=7

### Mutation Information

Mutation Type: Retroviral Insertion (OST)
Description: Retroviral insertion occurred in coding exon 4 (NCBI accession NM_{—}024263 .3).
1. Wild-type Expression Panel: Expression of the target gene was detected in all 13 adult tissue samples tested by RT-PCR, except skeletal muscle and bone.
2. QC Expression: RT-PCR analysis revealed that the transcript was absent in the (−/−) mouse analyzed (M-81). Disruption of the target gene was confirmed by Inverse PCR.

70.30.1. Phenotypic Analysis (for Disrupted Gene: DNA60618-1557 (UNQ662)

(a) Overall Phenotypic Summary:

Mutation of the gene encoding the ortholog of a human hypothetical protein (MGC3047) resulted in increased bone-related measurements in the (−/−) mice. Also, the (−/−) mice exhibited an increase in CD25 T cells in lymph nodes and an increase in CD38 non-lymphoid cells in Peyer's patches. Transcript was absent by RT-PCR.

(b) Immunology Phenotypic Analysis

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

T lymphocytes (T cells) are an important component of a mammalian immune response. T cells recognize antigens which are associated with a self-molecule encoded by genes within the major histocompatibility complex (MHC). The antigen may be displayed together with MHC molecules on the surface of antigen presenting cells, virus infected cells, cancer cells, grafts, etc. The T cell system eliminates these altered cells which pose a health threat to the host mammal. T cells include helper T cells and cytotoxic T cells. Helper T cells proliferate extensively following recognition of an antigen-MHC complex on an antigen presenting cell. Helper T cells also secrete a variety of cytokines, i.e., lymphokines, which play a central role in the activation of B cells, cytotoxic T cells and a variety of other cells which participate in the immune response.

In many immune responses, inflammatory cells infiltrate the site of injury or infection. The migrating cells may be neutrophilic, eosinophilic, monocytic or lymphocytic as can be determined by histologic examination of the affected tissues. Current Protocols in Immunology, ed. John E. Coligan, 1994, John Wiley & Sons, Inc.

Many immune related diseases are known and have been extensively studied. Such diseases include immune-mediated inflammatory diseases (such as rheumatoid arthritis, immune mediated renal disease, hepatobiliary diseases, inflammatory bowel disease (IBD), psoriasis, and asthma), non-immune-mediated inflammatory diseases, infectious diseases, immunodeficiency diseases, neoplasia, and graft rejection, etc. In the area of immunology, targets were identified for the treatment of inflammation and inflammatory disorders.

In the area of immunology, targets have been identified herein for the treatment of inflammation and inflammatory disorders. Immune related diseases, in one instance, could be treated by suppressing the immune response. Using neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

The following test was performed:

Flourescence-activated Cell-sorting (FACS) Analysis

Procedure:

FACS analysis of immune cell composition from peripheral blood was performed including CD4, CD8 and T cell receptor to evaluate T lymphocytes, CD19 for B lymphocytes, CD45 as a leukocyte marker and pan NK for natural killer cells. The FACS analysis was carried out on 2 wild type and 6 homozygous mice and included cells derived from thymus, spleen, bone marrow and lymph node.

In these studies, analyzed cells were isolated from thymus, peripheral blood, spleen, bone marrow and lymph nodes. Flow cytometry was designed to determine the relative proportions of CD4 and CD8 positive T cells, B cells, NK cells and monocytes in the mononuclear cell population. A Becton-Dickinson FACSCalibur 3-laser FACS machine was used to assess immune status. For Phenotypic Assays and Screening, this machine records CD4+/CD8−, CD8+/CD4−, NK, B cell and monocyte numbers in addition to the CD4+/CD8+ ratio. The mononuclear cell profile was derived by staining a single sample of lysed peripheral blood from each mouse with a panel of six lineage-specific antibodies: CD45 PerCP, anti-TCRb APC, CD4 PE, CD8 FITC, pan-NK PE, and CD19 FITC. The two FITC and PE labeled antibodies stain mutually exclusive cell types. The samples were analyzed using a Becton Dickinson FACSCalibur flow cytometer with CellQuest software.

Results:

FACS:

The (−/−) mice exhibited an increase in CD25 T cells in the lymph nodes and an increase in CD38 non-lymphoid cells in Peyer's patches when compared with their wildtype littermates.

These results show that mutant (−/−) mice show a phenotype marked by increased CD25 T cells as well as increased earlier stage T cells. Thus, the resultant mutant mice demonstrate a positive immunological phenotype and antagonists or inhibitors of PRO1293 polypeptides would be expected to mimic these results. Apparently, PRO1293 polypeptides act as a negative regulator for T cell activation. Thus, PRO1293 polypeptides or agonists thereof, would be beneficial as a negative regulator of T cell proliferation in those instances wherein a pronounced T-cell proliferation is present such as occurs for example in rheumatoid arthritis patients.

(c) Bone Metabolism & Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Bone MicroCT Analysis:

Procedure: MicroCT was also used to get very sensitive measurements of BMD. One vertebra and 1 femur were taken from a cohort of 4 wild type and 8 homozygous mice. Measurements were taken of lumbar 5 vertebra trabecular bone volume, trabecular thickness, connectivity density and midshaft femur total bone area and cortical thickness. The μCT40 scans provided detailed information on bone mass and architecture. Multiple bones were placed into sample holders and scanned automatically. Instrument software was used to select regions of interest for analysis. Trabecular bone parameters were analyzed in the fifth lumbar vertebrae (LV5) at 16 micrometer resolution and cortical bone parameters were analyzed in the femur midshaft at a resolution of 20 micrometers.

Results:

DEXA: The male (−/−) mice exhibited increased mean bone mineral content, volumetric bone mineral density, and bone mineral density in total body and femur when compared with their gender-matched (+/+) littermates and the historical means.
Micro-CT: The male (−/−) mice exhibited increased mean vertebral trabecular bone volume and number when compared with their gender-matched (+/+) littermates and the historical means.

In summary, the (−/−) mice exhibited increased bone mineral related measurements and vertebecular bone density measurements when compared with their gender-matched (+/+) littermates. These results indicate that the knockout mutant phenotype is associated with such bone abnormalities as osteopetrosis. Osteopetrosis is a condition characterized by abnormal thickening and hardening of bone and abnormal fragility of the bones. As such, PRO1293 polypeptides or agonists thereof would be beneficial for the treatment of osteopetrosis or other osteo-related diseases. On the other hand, inhibitors or antagonists of PRO1293 polypeptides would be useful in bone healing.

70.31. Generation and Analysis of Mice Comprising DNA47394-1572 (UNQ676) Gene Disruptions

In these knockout experiments, the gene encoding PRO1310 polypeptides (designated as DNA47394-1572) (UNQ676) was disrupted. The gene specific information for these studies is as follows: the mutated mouse gene corresponds to nucleotide reference: NM_{—}018867 ACCESSION:NM_{—}018867 NID:18466803 Mus musculus Mus musculus carboxypeptidase X 2 (M14 family) (Cpxm2); protein reference: Q9D2L5 ACCESSION:Q9D2L5 NID: Mus musculus (Mouse). Potential carboxypeptidase-like protein X2 precursor; the human gene sequence reference: NM_{—}198148 ACCESSION:NM_{—}198148 NID: gi 39930572 ref NM_{—}198148.1 Homo sapiens hypothetical protein LOC119587 (LOC119587); the human protein sequence corresponds to reference: Q8N436 ACCESSION:Q8N436 NID: Homo sapiens (Human). Potential carboxypeptidase-like protein X2 precursor.

The mouse gene of interest is Cpxm2 (carboxypeptidase X 2 [M14 family]), ortholog of human CPXM2 (carboxypeptidase-like protein X2). Aliases include Cpx2, CPX-2, 4632435C11Rik, carboxypeptidase X2, metallocarboxypeptidase 2, and carboxypeptidase Hlo.

CPXM2 is a likely secreted protein expressed in brain, liver, kidney, and lung. CPXM2 contains a signal peptide, a discoidin domain, and a vestigial metallocarboxypeptidase domain that is not likely to be catalytically active (Xin et al, DNA Cell Biol 17(10):897-909 (1998)). Discoidin domains are thought to be involved in binding to cell surface-attached carbohydrates or anionic phospholipids. Discoidin domains are found in blood coagulation factors V and VIII and in the slime mold cell adhesion protein discoidin. CPXM2 may be involved in cell adhesion (SMART accession SM00231; Lei et al, DNA Cell Biol 18(2):175-85 (1999)).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 19 | 36 | 17 | 72 |
| Expected | 18 | 36 | 18 | 72 |

Chi-Sq.=0.3 Significance=0.860708 (hom/n)=0.26 Avg. Litter Size=

### Mutation Information

Mutation Type: Homologous Recombination (standard)
Description: Coding exon 1 was targeted (NCBI accession AF017639.1).
1. Wild-type Expression Panel: Expression of the target gene was detected in embryonic stem (ES) cells and in all 13 adult tissue samples tested by RT-PCR.
2. QC Expression: Disruption of the target gene was confirmed by Southern hybridization analysis.

70.31.1. Phenotypic Analysis (for Disrupted Gene: DNA47394-1572 (UNQ676)

(a) Overall Phenotypic Summary:

Mutation of the gene encoding the ortholog of human carboxypeptidase X (M14 family), member 2 (CPXM2) resulted in impaired glucose tolerance in male (−/−) mice. The (−/−) mice also exhibited increased splenic CD25 T cells and peritoneal CD23 B cells. The homozygous mutant mice also exhibited increased mean serum TNF-alpha and IL-6 responses to LPS challenge. Levels of IgG2a were also elevated in the homozygous mice. Disruption of the target gene was confirmed by Southern hybridization analysis.

(b) Immunology Phenotypic Analysis

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

T lymphocytes (T cells) are an important component of a mammalian immune response. T cells recognize antigens which are associated with a self-molecule encoded by genes within the major histocompatibility complex (MHC). The antigen may be displayed together with MHC molecules on the surface of antigen presenting cells, virus infected cells, cancer cells, grafts, etc. The T cell system eliminates these altered cells which pose a health threat to the host mammal. T cells include helper T cells and cytotoxic T cells. Helper T cells proliferate extensively following recognition of an antigen-MHC complex on an antigen presenting cell. Helper T cells also secrete a variety of cytokines, i.e., lymphokines, which play a central role in the activation of B cells, cytotoxic T cells and a variety of other cells which participate in the immune response.

In many immune responses, inflammatory cells infiltrate the site of injury or infection. The migrating cells may be neutrophilic, eosinophilic, monocytic or lymphocytic as can be determined by histologic examination of the affected tissues. Current Protocols in Immunology, ed. John E. Coligan, 1994, John Wiley & Sons, Inc.

Many immune related diseases are known and have been extensively studied. Such diseases include immune-mediated inflammatory diseases (such as rheumatoid arthritis, immune mediated renal disease, hepatobiliary diseases, inflammatory bowel disease (IBD), psoriasis, and asthma), non-immune-mediated inflammatory diseases, infectious diseases, immunodeficiency diseases, neoplasia, and graft rejection, etc. In the area of immunology, targets were identified for the treatment of inflammation and inflammatory disorders.

In the area of immunology, targets have been identified herein for the treatment of inflammation and inflammatory disorders. Immune related diseases, in one instance, could be treated by suppressing the immune response. Using neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

The following test was performed:

Acute Phase Response:

Test Description: Bacterial lipopolysaccharide (LPS) is an endotoxin, and as such is a potent inducer of an acute phase response and systemic inflammation. The Level I LPS mice were injected intraperitoneally (i.p.) with a sublethal dose of LPS in 200 μL sterile saline using a 26 gauge needle. The doses were based on the average weight of the mice tested at 1 μg/g body weight 3 hours after injection; a 100u1 blood sample was then taken and analyzed for the presence of TNFα, MCP-1, and IL-6 on the FACSCalibur instrument.

Results:

The (−/−) mice exhibited increased mean serum TNF-alpha and IL-6 responses to LPS challenge when compared with their (+/+) littermates and the historical means.

In summary, the LPS endotoxin challenge demonstrated that knockout mice deficient in the gene encoding PRO1310 polypeptides exhibit immunological abnormalities when compared with their wild-type littermates. In particular, the mutant mice exhibited an increased ability to elicit an immunological response (TNF-alpha and IL-6 production) when challenged with the LPS endotoxin indicating a proinflammatory response. IL-6 contributes to the later stages of B cell activation. TNF-alpha is an important inflammatory mediator. In addition, both TNF-alpha and IL-6 play a critical role in inducing the acute phase response and systemic inflammation. TNF-alpha can substitute for the membrane-bound signal in macrophage activation (thus serving as an effector molecule). This suggests that inhibitors or antagonists to PRO1310 polypeptides would stimulate the immune system and would find utility in the cases wherein this effect would be beneficial to the individual such as in the case of leukemia, and other types of cancer, and in immunocompromised patients, such as AIDS sufferers. Accordingly, PRO1310 polypeptides or agonists thereof would be useful in inhibiting the immune response and would be useful candidates for suppressing harmful immune responses, e.g. in the case of graft rejection or graft-versus-host diseases.

Serum Immunoglobulin Isotyping Assay:

The Serum Immunoglobulin Isotyping Assay is performed using a Cytometric Bead Array (CBA) kit. This assay is used to rapidly identify the heavy and light chain isotypes of a mouse monoclonal antibody in a single sample. The values expressed are “relative fluorescence units” and are based on the detection of kappa light chains. Any value <6 is not significant.

Results:

Serum Immunoglobulin isotyping resulted in the observation that (−/−) mice exhibited a two-fold increase in serum IgG2a compared to the (+/+) littermates and the historical means.

Mutant (−/−) mice exhibited elevation of IgG2a serum immunoglobulins compared to their gender-matched (+/+) littermates. IgG2a immunoglobulins have neutralization effects and to a lesser extent are important for activation of the complement system. The observed phenotype suggests that the PRO1310 polypeptide is a negative regulator of inflammatory responses. These immunological abnormalities suggest that inhibitors (antagonists) of PRO1310 polypeptides would be important agents which could stimulate the immune system (such as T cell proliferation) and would find utility in the cases wherein this effect would be beneficial to the individual such as in the case of leukemia, and other types of cancer, and in immunocompromised patients, such as AIDS sufferers. Accordingly, PRO1310 polypeptides or agonists thereof would be useful in inhibiting the immune response and would be useful candidates for suppressing harmful immune responses, e.g. in the case of graft rejection or graft-versus-host diseases.

Flourescence-activated Cell-sorting (FACS) Analysis

Procedure:

FACS analysis of immune cell composition from peripheral blood was performed including CD4, CD8 and T cell receptor to evaluate T lymphocytes, CD19 for B lymphocytes, CD45 as a leukocyte marker and pan NK for natural killer cells. The FACS analysis was carried out on 2 wild type and 6 homozygous mice and included cells derived from thymus, spleen, bone marrow and lymph node.

In these studies, analyzed cells were isolated from thymus, peripheral blood, spleen, bone marrow and lymph nodes. Flow cytometry was designed to determine the relative proportions of CD4 and CD8 positive T cells, B cells, NK cells and monocytes in the mononuclear cell population. A Becton-Dickinson FACSCalibur 3-laser FACS machine was used to assess immune status. For Phenotypic Assays and Screening, this machine records CD4+/CD8−, CD8+/CD4−, NK, B cell and monocyte numbers in addition to the CD4+/CD8+ ratio. The mononuclear cell profile was derived by staining a single sample of lysed peripheral blood from each mouse with a panel of six lineage-specific antibodies: CD45 PerCP, anti-TCRb APC, CD4 PE, CD8 FITC, pan-NK PE, and CD19 FITC. The two FITC and PE labeled antibodies stain mutually exclusive cell types. The samples were analyzed using a Becton Dickinson FACSCalibur flow cytometer with CellQuest software.

Results:

FACS:

The homozygous (−/−) mice exhibited an increase in CD25 T cells in lymph node and spleen and peritoneal CD23 B cells compared with their gender-matched (+/+) littermates and the historical means.

These results show that mutant (−/−) mice show a phenotype marked by increased CD25 T cells and CD23 B cells. Thus, the resultant mutant mice demonstrate a positive immunological phenotype and antagonists or inhibitors of PRO1312 polypeptides would be expected to mimic these results. Apparently, PRO1312 polypeptides act as a negative regulator for T cell activation.

(c) Phenotypic Analysis: Metabolism-Blood Chemistry/Glucose Tolerance

In the area of metabolism, targets may be identified for the treatment of diabetes. Blood chemistry phenotypic analysis includes blood glucose measurements. The COBAS Integra 400 (mfr: Roche) was used for running blood chemistry tests on the mice. In the area of metabolism, targets may be identified for the treatment of diabetes. Blood chemistry phenotypic analysis includes glucose tolerance tests to measure insulin sensitivity and changes in glucose metabolism. Abnormal glucose tolerance test results may indicate but may not be limited to the following disorders or conditions: Diabetes Type 1 and Type 2, Syndrome X, various cardiovascular diseases and/or obesity.

Procedure: A cohort of 2 wild type and 4 homozygous mice were used in this assay. The glucose tolerance test is the standard for defining impaired glucose homeostasis in mammals. Glucose tolerance tests were performed using a Lifescan glucometer. Animals were injected IP at 2 g/kg with D-glucose delivered as a 20% solution and blood glucose levels were measured at 0, 30, 60 and 90 minutes after injection.

Results:

Glucose Tolerance Test:

The male (−/−) mice exhibited impaired glucose tolerance at T-60 and T-90 when compared with their gender-matched (+/+) littermates and the historical means.

These studies indicated that (−/−) mice exhibit a decreased or impaired glucose tolerance in the presence of normal fasting glucose at T-60 and T-90 intervals tested when compared with their gender-matched (+/+) littermates and the historical means. Thus, knockout mutant mice exhibited the phenotypic pattern of an impaired glucose homeostasis, and therefor PRO1310 polypeptides (or agonists thereof) or its encoding gene would be useful in the treatment of conditions associated with an impaired glucose homeostasis and/or various cardiovascular diseases, including diabetes.

70.32. Generation and Analysis of Mice Comprising DNA61873-1574 (UNQ678) Gene Disruptions

In these knockout experiments, the gene encoding PRO1312 polypeptides (designated as DNA61873-1574) (UNQ678) was disrupted. The gene specific information for these studies is as follows: the mutated mouse gene corresponds to nucleotide reference: NM_{—}020626 ACCESSION:NM_{—}020626 NID:10181181 Mus musculus Mus musculus kidney-specific membrane protein (Nx17-pending); protein reference: Q9ESG4 ACCESSION:Q9ESG4 NID: Mus musculus (Mouse). KIDNEY-SPECIFIC MEMBRANE PROTEIN NX-17 (0610008J07RIK PROTEIN); the human gene sequence reference: NM_{—}020665 ACCESSION:NM_{—}020665 NID:21361864 Homo sapiens Homo sapiens kidney-specific membrane protein (NX-17); the human protein sequence corresponds to reference: Q9HBJ8 ACCESSION:Q9HBJ8 NID: Homo sapiens (Human). KIDNEY-SPECIFIC MEMBRANE PROTEIN NX-17 (HYPOTHETICAL 25.2 KDA PROTEIN).

The mouse gene of interest is Tmem27 (transmembrane protein 27), ortholog of human TMEM27. Aliases include NX17, NX-17, NX=17, collectrin, 0610008J07Rik, and kidney-specific membrane protein.

TMEM27 is a transmembrane glycoprotein expressed on the luminal plasma membrane of the renal collecting duct. The protein is structurally similar to angiotensin converting enzyme 2 but is catalytically inactive, lacking a dipeptidyl carboxypeptidase catalytic domain. TMEM27 mRNA is upregulated during development and in hypertrophic kidneys after renal ablation, suggesting that TMEM27 may play a role in renal organogenesis and progressive renal failure (Zhang et al, J Biol Chem 276(20):17132-9 (2001)). This project is X-linked.

### Summary of X-linked Gene Distribution by Sex and Genotype

(Only the agouti pups from the male chimeras are included.)

Summary of X-linked Gene Distributions for Sex by Genotype

| | | | |
|---|---|---|---|
| | Agouti F1 | | F1a |
| Progeny | (M chimera × wt) | Progeny | (F het × wt) |

| Sex | wt | het | Sex | wt | het | hemi |
|---|---|---|---|---|---|---|
| M | 7 | 0 | M | 25 | n/a | 17 |
| F | 0 | 4 | F | 13 | 19 | n/a |

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 20 | 19 | 39 | 78 |
| Expected | 19.5 | 39 | 19.5 | 78 |

Chi-Sq.=101.56 Significance=8.841516E-23 (hom/n)=0.59 Avg. Litter Size=8

### Mutation Information

Mutation Type: Homologous Recombination (standard)
Description: Coding exons 1 and 2 were targeted (NCBI accession NM_{—}020626. 1).
1. Wild-type Expression Panel: Expression of the target gene was detected in embryonic stem (ES) cells and in all 13 adult tissue samples tested by RT-PCR, except lung; bone; stomach, small intestine, and colon; and heart.
2. QC Expression: Disruption of the target gene was confirmed by Southern hybridization analysis.

70.32.1. Phenotypic Analysis (for Disrupted Gene: DNA61873-1574 (UNQ678)

(a) Overall Phenotypic Summary:

Mutation of the gene encoding the ortholog of human transmembrane protein 27 (TMEM27) resulted in the observation that female homozygous and male hemizygous mutant mice exhibited a decreased depressive-like response during tail suspension testing when compared with their wild-type littermates and the historical mean. Sporadic flecks of depigmentation were also noted on the retinas of some female heterozygous and homozygous mutants and one male hemizygous mutant. In addition, an increase in splenic CD25 T cells and peritoneal CD23 B cells was noted in the homozygous mice. Disruption of the target gene was confirmed by Southern hybridization analysis. This mutation is in an X-linked gene. The male hemizygous (wild-type) and hemizygous mutant mice are designated as (+/+) and (−/−), respectively.

(b) Phenotypic Analysis: CNS/Neurology

In the area of neurology, analysis focused herein on identifying in vivo validated targets for the treatment of neurological and psychiatric disorders including depression, generalized anxiety disorders, attention deficit hyperactivity disorder, obsessive compulsive disorder, schizophrenia, cognitive disorders, hyperalgesia and sensory disorders. Neurological disorders include the category defined as “anxiety disorders” which include but are not limited to: mild to moderate anxiety, anxiety disorder due to a general medical condition, anxiety disorder not otherwise specified, generalized anxiety disorder, panic attack, panic disorder with agoraphobia, panic disorder without agoraphobia, posttraumatic stress disorder, social phobia, specific phobia, substance-induced anxiety disorder, acute alcohol withdrawal, obsessive compulsive disorder, agoraphobia, bipolar disorder I or II, bipolar disorder not otherwise specified, cyclothymic disorder, depressive disorder, major depressive disorder, mood disorder, substance-induced mood disorder. In addition, anxiety disorders may apply to personality disorders including but not limited to the following types: paranoid, antisocial, avoidant behavior, borderline personality disorders, dependent, histronic, narcissistic, obsessive-compulsive, schizoid, and schizotypal.

Procedure:

Behavioral screens were performed on a cohort of 4 wild type, 4 heterozygous and 8 homozygous mice. All behavioral tests were done between 12 and 16 weeks of age unless reduced viability necessitates earlier testing. These tests included open field to measure anxiety, activity levels and exploration.

Functional Observational Battery (FOB) Test—Tail Suspension Testing:

The FOB is a series of situations applied to the animal to determine gross sensory and motor deficits. A subset of tests from the Irwin neurological screen that evaluates gross neurological function is used. In general, short-duration, tactile, olfactory, and visual stimuli are applied to the animal to determine their ability to detect and respond normally. These simple tests take approximately 10 minutes and the mouse is returned to its home cage at the end of testing.

Tail Suspension Testing:

The tail suspension test is a procedure that has been developed as a model for depressive-like behavior in rodents. In this particular setup, a mouse is suspended by its tail for 6 minutes, and in response the mouse will struggle to escape from this position. After a certain period of time the struggling of the mouse decreases and this is interpreted as a type of learned helplessness paradigm. Animals with invalid data (i.e. climbed their tail during the testing period) are excluded from analysis.

Results:

Response to Helplessness: The female (−/−) and male (0/−) mice exhibited a decreased median immobility time during tail suspension testing when compared with their wild-type littermates and the historical mean, suggesting a decreased depressive-like response. In summary, the tail suspension testing revealed a phenotype associated with increased anxiety which could be associated with mild to moderate anxiety, anxiety due to a general medical condition, and/or bipolar disorders; hyperactivity; sensory disorders; obsessive-compulsive disorders, schizophrenia or a paranoid personality. Thus, PRO1312 polypeptides or agonists thereof would be useful in the treatment of such neurological disorders.

(c) Cardiovascular Phenotypic Analysis:

In the area of cardiovascular biology, phenotypic testing was performed to identify potential targets for the treatment of cardiovascular, endothelial or angiogenic disorders. One such phenotypic test included optic fundus photography and angiography to determine the retinal arteriovenous ratio (A/V ratio) in order to flag various eye abnormalities. An abnormal A/V ratio signals such systemic diseases or disorders that may be related to the vascular disease of hypertension (and any disease that causes hypertension, e.g. atherosclerosis), diabetes or other ocular diseases corresponding to ophthalmological disorders. Such eye abnormalities may include but are not limited to the following: retinal abnormality is retinal dysplasia, various retinopathies, restenosis, retinal artery obstruction or occlusion; retinal degeneration causing secondary atrophy of the retinal vasculature, retinitis pigmentosa, macular dystrophies, Stargardt's disease, congenital stationary night blindness, choroideremia, gyrate atrophy, Leber's congenital amaurosis, retinoschisis disorders, Wagner's syndrome, Usher syndromes, Zellweger syndrome, Saldino-Mainzer syndrome, Senior-Loken syndrome, Bardet-Biedl syndrome, Alport's syndrome, Alstom's syndrome, Cockayne's syndrome, dysplaisa spondyloepiphysaria congentia, Flynn-Aird syndrome, Friedreich ataxia, Hallgren syndrome, Marshall syndrome, Albers-Schnoberg disease, Refsum's disease, Kearns-Sayre syndrome, Waardenburg's syndrome, Alagile syndrome, myotonic dystrophy, olivopontocerebellar atrophy, Pierre-Marie dunsdrome, Stickler syndrome, carotinemeia, cystinosis, Wolfram syndrome, Bassen-Kornzweig syndrome, abetalipoproteinemia, incontinentia pigmenti, Batten's disease, mucopolysaccharidoses, homocystinuria, or mannosidosis.

Procedure: A cohort of 4 wild type, 4 heterozygous and 8 homozygous mice were tested in this assay. Optic fundus photography was performed on conscious animals using a Kowa Genesis small animal fundus camera modified according to Hawes and coauthors (Hawes et al., 1999 Molecular Vision 1999; 5:22). Intra-peritoneal injection of fluorescein permitted the acquisition of direct light fundus images and fluorescent angiograms for each examination. In addition to direct ophthalmological changes, this test can detect retinal changes associated with systemic diseases such as diabetes and atherosclerosis or other retinal abnormalities. Pictures were provided of the optic fundus under normal light. The angiographic pictures allowed examination of the arteries and veins of the eye. In addition an artery to vein (A/V) ratio was determined for the eye.

Ophthalmology analysis was performed on generated F2 wild type, heterozygous, and homozygous mutant progeny using the protocol described above. Specifically, the A/V ratio was measured and calculated according to the fundus images with Kowa COMIT+ software. This test takes color photographs through a dilated pupil: the images help in detecting and classifying many diseases. The artery to vein ratio (A/V) is the ratio of the artery diameter to the vein diameter (measured before the bifurcation of the vessels). Many diseases will influence the ratio, i.e., diabetes, cardiovascular disorders, papilledema, optic atrophy or other eye abnormalities such as retinal degeneration (known as retinitis pigmentosa) or retinal dysplasia, vision problems or blindness. Thus, phenotypic observations which result in an increased artery-to-vein ratio in homozygous (−/−) and heterozygous (+/−) mutant progeny compared to wildtype (+/+) littermates would be indicative of such pathological conditions.

Results:

Fundus: Sporadic flecks of depigmentation were noted on the retinas of 2/4 (−/−) mice (F-50 and F-84), 1/4 (0/−) mice (M-59), and 2/4 (+/−) mice (F-60 and F-79). Retinal depigmentation may be associated with retinal degeneration.

In summary, in this study, (−/−) mice showed opthamological abnormalities which would lead to depigmentation of retinal vessels and retinal degeneration when compared with their (+/+) littermates. In summary, by knocking out the gene identified as DNA61873-1574 encoding PRO1312 polypeptides, homozygous mutant progeny exhibit phenotypes which are associated with retinal artery abnormalities. Such detected retinal changes are most commonly associated with cardiovascular systemic diseases or disorders that may be related to the vascular disease of hypertension (and any disease that causes hypertension, e.g. atherosclerosis), diabetes or other ocular diseases corresponding to ophthalmological disorders such as retinal degeneration. Thus, antagonists of PRO1312encoding genes would lead to similar pathological retinal changes, whereas agonists would be useful as therapeutic agents in the treatment of hypertension, atherosclerosis or other opthamological disorders including retinal degeneration and diseases associated with this condition (as indicated above).

(d) Immunology Phenotypic Analysis

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

T lymphocytes (T cells) are an important component of a mammalian immune response. T cells recognize antigens which are associated with a self-molecule encoded by genes within the major histocompatibility complex (MHC). The antigen may be displayed together with MHC molecules on the surface of antigen presenting cells, virus infected cells, cancer cells, grafts, etc. The T cell system eliminates these altered cells which pose a health threat to the host mammal. T cells include helper T cells and cytotoxic T cells. Helper T cells proliferate extensively following recognition of an antigen-MHC complex on an antigen presenting cell. Helper T cells also secrete a variety of cytokines, i.e., lymphokines, which play a central role in the activation of B cells, cytotoxic T cells and a variety of other cells which participate in the immune response.

In many immune responses, inflammatory cells infiltrate the site of injury or infection. The migrating cells may be neutrophilic, eosinophilic, monocytic or lymphocytic as can be determined by histologic examination of the affected tissues. Current Protocols in Immunology, ed. John E. Coligan, 1994, John Wiley & Sons, Inc.

Many immune related diseases are known and have been extensively studied. Such diseases include immune-mediated inflammatory diseases (such as rheumatoid arthritis, immune mediated renal disease, hepatobiliary diseases, inflammatory bowel disease (IBD), psoriasis, and asthma), non-immune-mediated inflammatory diseases, infectious diseases, immunodeficiency diseases, neoplasia, and graft rejection, etc. In the area of immunology, targets were identified for the treatment of inflammation and inflammatory disorders.

In the area of immunology, targets have been identified herein for the treatment of inflammation and inflammatory disorders. Immune related diseases, in one instance, could be treated by suppressing the immune response. Using neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

The following test was performed:

Flourescence-activated Cell-sorting (FACS) Analysis

Procedure:

FACS analysis of immune cell composition from peripheral blood was performed including CD4, CD8 and T cell receptor to evaluate T lymphocytes, CD19 for B lymphocytes, CD45 as a leukocyte marker and pan NK for natural killer cells. The FACS analysis was carried out on 2 wild type and 6 homozygous mice and included cells derived from thymus, spleen, bone marrow and lymph node.

In these studies, analyzed cells were isolated from thymus, peripheral blood, spleen, bone marrow and lymph nodes. Flow cytometry was designed to determine the relative proportions of CD4 and CD8 positive T cells, B cells, NK cells and monocytes in the mononuclear cell population. A Becton-Dickinson FACSCalibur 3-laser FACS machine was used to assess immune status. For Phenotypic Assays and Screening, this machine records CD4+/CD8−, CD8+/CD4−, NK, B cell and monocyte numbers in addition to the CD4+/CD8+ ratio. The mononuclear cell profile was derived by staining a single sample of lysed peripheral blood from each mouse with a panel of six lineage-specific antibodies: CD45 PerCP, anti-TCRb APC, CD4 PE, CD8 FITC, pan-NK PE, and CD19 FITC. The two FITC and PE labeled antibodies stain mutually exclusive cell types. The samples were analyzed using a Becton Dickinson FACSCalibur flow cytometer with CellQuest software.

Results:

FACS:

The homozygous (−/−) mice exhibited an increase in CD25 T cells in spleen and an increase in peritoneal CD23 B cells compared with their gender-matched (+/+) littermates and the historical means. These results show that mutant (−/−) mice exhibit a phenotype marked by increased CD25 T cells as well as increased mature B cells. Thus, the resultant mutant mice demonstrate a positive immunological phenotype and antagonists or inhibitors of PRO1312 polypeptides would be expected to mimic these results. Apparently, PRO1312 polypeptides act as a negative regulator for both T cell activation and B cell proliferation.

70.33. Generation and Analysis of Mice Comprising DNA62812-1594 (UNQ690) Gene Disruptions

In these knockout experiments, the gene encoding PRO1335 polypeptides (designated as DNA62812-1594) (UNQ690) was disrupted. The gene specific information for these studies is as follows: the mutated mouse gene corresponds to nucleotide reference: NM_{—}011797 ACCESSION:NM_{—}011797 NID: 6753263 Mus musculus Mus musculus carbonic anhydrase 14 (Car14); protein reference: Q9WVT6 ACCESSION:Q9WVT6 NID: Mus musculus (Mouse). CARBONIC ANHYDRASE XIV PRECURSOR (EC 4.2.1.1) (CARBONATE DEHYDRATASE XIV) (CA-XIV); the human gene sequence reference: NM_{—}012113 ACCESSION:NM_{—}012113 NID:6912283 Homo sapiens Homo sapiens carbonic anhydrase XIV (CA14); the human protein sequence corresponds to reference: Q9ULX7 ACCESSION:Q9ULX7 NID: Homo sapiens (Human). CARBONIC ANHYDRASE XIV PRECURSOR (EC 4.2.1.1) (CARBONATE DEHYDRATASE XIV) (CA-XIV).

The mouse gene of interest is Car14 (carbonic anhydrase 14), ortholog of human CA14 (carbonic anhydrase XIV). Aliases include carbonic dehydratase.

CA14 is a type I plasma membrane protein and enzyme that catalyzes the reversible hydration of carbon dioxide to form carbonic acid. The protein consists of a signal peptide, an extracellular catalytic domain, a transmembrane segment, and a short cytoplasmic C terminus. CA14 is expressed at high levels in the proximal convoluted tubule and in other tissues, such as heart, skeletal muscle, brain, lung, and liver. CA14 is likely to be involved in carbon dioxide metabolism and acid-base balance (Fujikawa-Adachi et al, Genomics 61(1):74-81 (1999); Mori et al, J Biol Chem 274(22):15701-5 (1999); Parkkila et al, BMC Gastroenterol 2(1):13 (2002); Whittington et al, J Biol Chem 279(8):7223-8 (2004)).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 16 | 35 | 24 | 75 |
| Expected | 18.75 | 37.5 | 18.75 | 75 |

Chi-Sq.=0.12 Significance=0.94176453 (hom/n)=0.24 Avg. Litter Size=9

### Mutation Information

Mutation Type: Retroviral Insertion (OST)
Description: Retroviral insertion occurred in the intron between coding exons 1 and 2 (NCBI accession NM_{—}011797.1).
1. Wild-type Expression Panel: Expression of the target gene was detected in all 13 adult tissue samples tested by RT-PCR, except spleen; liver; bone; stomach, small intestine, and colon; and adipose.
2. QC Expression: RT-PCR analysis revealed that the transcript was absent in the (−/−) mouse analyzed (M-184). Disruption of the target gene was confirmed by Inverse PCR.

70.33.1. Phenotypic Analysis (for Disrupted Gene: DNA62812-1594 (UNQ690)

(a) Overall Phenotypic Summary:

Mutation of the gene encoding the ortholog of human carbonic anhydrase XIV (CA14) resulted in increased serum TNF-alpha, MCP-1 and IL-6 responses to LPS challenge in (−/−) mice. Female knockouts showed significant increase in uric acid levels (p=0.01198). RT-PCR analysis revealed that the transcript was absent in the homozygous mutant mice.

(b) Immunology Phenotypic Analysis

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

T lymphocytes (T cells) are an important component of a mammalian immune response. T cells recognize antigens which are associated with a self-molecule encoded by genes within the major histocompatibility complex (MHC). The antigen may be displayed together with MHC molecules on the surface of antigen presenting cells, virus infected cells, cancer cells, grafts, etc. The T cell system eliminates these altered cells which pose a health threat to the host mammal. T cells include helper T cells and cytotoxic T cells. Helper T cells proliferate extensively following recognition of an antigen-MHC complex on an antigen presenting cell. Helper T cells also secrete a variety of cytokines, i.e., lymphokines, which play a central role in the activation of B cells, cytotoxic T cells and a variety of other cells which participate in the immune response.

In many immune responses, inflammatory cells infiltrate the site of injury or infection. The migrating cells may be neutrophilic, eosinophilic, monocytic or lymphocytic as can be determined by histologic examination of the affected tissues. Current Protocols in Immunology, ed. John E. Coligan, 1994, John Wiley & Sons, Inc.

Many immune related diseases are known and have been extensively studied. Such diseases include immune-mediated inflammatory diseases (such as rheumatoid arthritis, immune mediated renal disease, hepatobiliary diseases, inflammatory bowel disease (IBD), psoriasis, and asthma), non-immune-mediated inflammatory diseases, infectious diseases, immunodeficiency diseases, neoplasia, and graft rejection, etc. In the area of immunology, targets were identified for the treatment of inflammation and inflammatory disorders.

In the area of immunology, targets have been identified herein for the treatment of inflammation and inflammatory disorders. Immune related diseases, in one instance, could be treated by suppressing the immune response. Using neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

The following test was performed:

Acute Phase Response:

Test Description: Bacterial lipopolysaccharide (LPS) is an endotoxin, and as such is a potent inducer of an acute phase response and systemic inflammation. The Level I LPS mice were injected intraperitoneally (i.p.) with a sublethal dose of LPS in 200 μL sterile saline using a 26 gauge needle. The doses were based on the average weight of the mice tested at 1 μg/g body weight 3 hours after injection; a 100 ul blood sample was then taken and analyzed for the presence of TNFa, MCP-1, and IL-6 on the FACSCalibur instrument.

Results:

The (−/−) mice exhibited increased mean serum TNF-alpha, MCP-1 and IL-6 responses to LPS challenge when compared with their (+/+) littermates and the historical means.

In summary, the LPS endotoxin challenge demonstrated that knockout mice deficient in the gene encoding PRO1335 polypeptides exhibit immunological abnormalities when compared with their wild-type littermates. In particular, the mutant mice exhibited an increased ability to elicit an immunological response (TNF-alpha, MCP-1 and IL-6 production) when challenged with the LPS endotoxin indicating a proinflammatory response. TNF-alpha, MCP-1 and IL-6 contribute to the later stages of B cell activation. TNF-alpha is an important inflammatory mediator. In addition, both TNF-alpha, MCP-1 and IL-6 play a critical role in inducing the acute phase response and systemic inflammation. TNF-alpha can substitute for the membrane-bound signal in macrophage activation (thus serving as an effector molecule). This suggests that inhibitors or antagonists to PRO1335 polypeptides would stimulate the immune system and would find utility in the cases wherein this effect would be beneficial to the individual such as in the case of leukemia, and other types of cancer, and in immunocompromised patients, such as AIDS sufferers. Accordingly, PRO1335 polypeptides or agonists thereof would be useful in inhibiting the immune response and would be useful candidates for suppressing harmful immune responses, e.g. in the case of graft rejection or graft-versus-host diseases.

(c) Phenotypic Analysis: Metabolism-Blood Chemistry

In the area of metabolism, targets may be identified for the treatment of metabolic disorders. The COBAS Integra 400 (mfr: Roche) was used for running blood chemistry tests on the mice. In addition to measuring blood glucose levels the following blood chemistry tests are also routinely performed: Alkaline Phosphatase; Alanine Amino-Transferase; Albumin; Bilirubin; Phosphorous; Creatinine; BUN=Blood Urea Nitrogen; Calcium; Uric Acid; Sodium; Potassium; and Chloride.

Results:

Female (−/−) mice exhibited a notably increased uric acid level (p=0.01198) when compared with their gender-matched (+/+) littermates and the historical means.

Thus, mutant (−/−) mice exhibit a negative phenotype associated with a notably elevated uric acid level in the blood which is indicative of renal calculi (and associated kidney diseases) which is common in a type of gout (abnormal purine metabolism). PRO1335 polypeptides and agonists thereof would be useful in the treatment of such diseases associated with formation of renal calculi and/or abnormal purine metabolism.

70.34. Generation and Analysis of Mice Comprising DNA66669-1597 (UNQ694) Gene Disruptions

In these knockout experiments, the gene encoding PRO1339 polypeptides (designated as DNA66669-1597) (UNQ694) was disrupted. The gene specific information for these studies is as follows: the mutated mouse gene corresponds to nucleotide reference: NMO27926 Mus musculus carboxypeptidase A4 (Cpa4); protein reference: Q6P8K₈ ACCESSION:Q6P8K₈ NID: Mus musculus (Mouse). Carboxypeptidase A4; the human gene sequence reference: NM_{—}016352 ACCESSION:NM016352 NID: 10047105 Homo sapiens Homo sapiens carboxypeptidase A3 (L0051200); the human protein sequence corresponds to reference: Q9UI42 ACCESSION:Q9UI42 NID: Homo sapiens (Human). CARBOXYPEPTIDASE A4 PRECURSOR (EC 3.4.17.-) (CARBOXYPEPTIDASE A3).

The mouse gene of interest is Cpa4 (carboxypeptidase A4), ortholog of human CPA4. Aliases include 1110019K20Rik, CPA3, and carboxypeptidase A3. CPA4 is a putative secreted metalloprotease that catalyzes the cleavage of amino acids from the C terminus of proteins. CPA4 contains a signal peptide, an amino-terminal activation segment, and a zinc carboxypeptidase catalytic domain. Thus, the protein is likely to be secreted as a zymogen that is activated upon cleavage of the activation segment. CPA4 may be involved in differentiation and is a candidate gene for prostate cancer aggressiveness (Huang et al, Cancer Res 59(12):2981-8 (1999); Kayashima et al, Hum Genet. 112(3):220-6 (2003); Bentley et al, J Med Genet. 40(4):249-56 (2003)).

Targeted or gene trap mutations are generated in strain 129SvEv^{Brd}-derived embryonic stem (ES) cells.

The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 13 | 41 | 18 | 72 |
| Expected | 18 | 36 | 18 | 72 |

Chi-Sq.=6.48 Significance=0.039163895 (hom/n)=0.29 Avg. Litter Size=7

### Mutation Information

Mutation Type: Homologous Recombination (standard)
Description: Coding exons 5 through 7 were targeted (NCBI accession NM_{—}027926. 1).
1. Wild-type Expression Panel: Expression of the target gene was detected in embryonic stem (ES) cells and, among the 13 adult tissue samples tested by RT-PCR, in brain; spinal cord; eye; thymus; lung; and stomach, small intestine, and colon.
2. QC Expression: Disruption of the target gene was confirmed by Southern hybridization analysis.

70.34.1. Phenotypic Analysis (for Disrupted Gene: DNA66669-1597 (UNQ694)

(a) Overall Phenotypic Summary:

Mutation of the gene encoding the ortholog of human carboxypeptidase A4 (CPA4) resulted in an increase in IL-6 in response to LPS. In addition, decreased levels of IgM and IgG3 levels were noted in the (−/−) mice. Gene disruption was confirmed by Southern blot.

(b) Immunology Phenotypic Analysis

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

T lymphocytes (T cells) are an important component of a mammalian immune response. T cells recognize antigens which are associated with a self-molecule encoded by genes within the major histocompatibility complex (MHC). The antigen may be displayed together with MHC molecules on the surface of antigen presenting cells, virus infected cells, cancer cells, grafts, etc. The T cell system eliminates these altered cells which pose a health threat to the host mammal. T cells include helper T cells and cytotoxic T cells. Helper T cells proliferate extensively following recognition of an antigen-MHC complex on an antigen presenting cell. Helper T cells also secrete a variety of cytokines, i.e., lymphokines, which play a central role in the activation of B cells, cytotoxic T cells and a variety of other cells which participate in the immune response.

In many immune responses, inflammatory cells infiltrate the site of injury or infection. The migrating cells may be neutrophilic, eosinophilic, monocytic or lymphocytic as can be determined by histologic examination of the affected tissues. Current Protocols in Immunology, ed. John E. Coligan, 1994, John Wiley & Sons, Inc.

Many immune related diseases are known and have been extensively studied. Such diseases include immune-mediated inflammatory diseases (such as rheumatoid arthritis, immune mediated renal disease, hepatobiliary diseases, inflammatory bowel disease (IBD), psoriasis, and asthma), non-immune-mediated inflammatory diseases, infectious diseases, immunodeficiency diseases, neoplasia, and graft rejection, etc. In the area of immunology, targets were identified for the treatment of inflammation and inflammatory disorders.

In the area of immunology, targets have been identified herein for the treatment of inflammation and inflammatory disorders. Immune related diseases, in one instance, could be treated by suppressing the immune response. Using neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

The following test was performed:

Acute Phase Response:

Test Description Bacterial lipopolysaccharide (LPS) is an endotoxin, and as such is a potent inducer of an acute phase response and systemic inflammation. The Level I LPS mice were injected intraperitoneally (i.p.) with a sublethal dose of LPS in 200 μL sterile saline using a 26 gauge needle. The doses were based on the average weight of the mice tested at 1 μg/g body weight 3 hours after injection; a 100 ul blood sample was then taken and analyzed for the presence of TNFa, MCP-1, and IL-6 on the FACSCalibur instrument.

Results:

The (−/−) mice exhibited increased mean serum IL-6 responses to LPS challenge when compared with their (+/+) littermates and the historical means.

In summary, the LPS endotoxin challenge demonstrated that knockout mice deficient in the gene encoding PRO1339 polypeptides exhibit immunological abnormalities when compared with their wild-type littermates. In particular, the mutant mice exhibited an increased ability to elicit an immunological response (IL-6 production) when challenged with the LPS endotoxin indicating a proinflammatory response. IL-6 contributes to the later stages of B cell activation. In addition, IL-6 plays a critical role in inducing the acute phase response and systemic inflammation. This suggests that inhibitors or antagonists to PRO1339 polypeptides would stimulate the immune system and would find utility in the cases wherein this effect would be beneficial to the individual such as in the case of leukemia, and other types of cancer, and in immunocompromised patients, such as AIDS sufferers. Accordingly, PRO1339 polypeptides or agonists thereof would be useful in inhibiting the immune response and would be useful candidates for suppressing harmful immune responses, e.g. in the case of graft rejection or graft-versus-host diseases.

Serum Immunoglobulin Isotyping Assay:

The Serum Immunoglobulin Isotyping Assay is performed using a Cytometric Bead Array (CBA) kit. This assay is used to rapidly identify the heavy and light chain isotypes of a mouse monoclonal antibody in a single sample. The values expressed are “relative fluorescence units” and are based on the detection of kappa light chains. Any value <6 is not significant.

Results:

Serum Immunooglobulin isotyping resulted in the observation that (−/−) mice exhibited a five-fold decrease in serum IgM and less than 2 fold decrease in IgG3 compared to the (+/+) littermates and the historical means.

Mutant (−/−) mice exhibited decreased IgM and IgG3 serum immunoglobulins compared to their gender-matched (+/+) littermates. IgM immunoglobulins are the first to be produced in a humoral immune response for neutralization of bacterial toxins and are particularly important in activating the complement system. Likewise, IgG3 immunoglobulins have neutralization effects and to a lesser extent are important for activation of the complement system. The observed phenotype suggests that the PRO1339 polypeptide is a regulator of inflammatory responses. These immunological abnormalities suggest that PRO1339 polypeptides or agonists thereof would be important agents which would stimulate the immune system (such as T cell proliferation) and would find utility in the cases wherein this effect would be beneficial to the individual such as in the case of leukemia, and other types of cancer, and in immunocompromised patients, such as AIDS sufferers. Accordingly, antagonists (or inhibitors) of PRO1339 polypeptides would be useful in inhibiting the immune response and would be useful candidates for suppressing harmful immune responses, e.g. in the case of graft rejection or graft-versus-host diseases.

Hematology Analysis:

Test Description: Blood tests are carried out by Abbott's Cell-Dyn 3500R, an automated hematology analyzer. Some of its features include a five-part WBC differential. ‘Patient’ reports can cover over 22 parameters in all.

Results:

The (−/−) mice exhibited an increased red blood cell distribution width when compared with their (+/+) littermates and the historical means. These results are indicative of an abnormality in the composition of the red blood cells due to an increased RBC volume.

70.35. Generation and Analysis of Mice Comprising DNA88062 (UNQ696) Gene Disruptions

In these knockout experiments, the gene encoding PRO2155 polypeptides (designated as DNA88062) (UNQ696) was disrupted. The gene specific information for these studies is as follows: the mutated mouse gene corresponds to nucleotide reference: NM_{—}009654 Mus musculus albumin 1 (Alb1); protein reference: P07724 ACCESSION:P07724 NID: Mus musculus (Mouse). SERUM ALBUMIN PRECURSOR; the human gene sequence reference: NM_{—}000477 ACCESSION:NM000477 NID: 8392890 Homo sapiens Homo sapiens albumin (ALB); the human protein sequence corresponds to reference: P02768 ACCESSION:P02768 NID: Homo sapiens (Human). SERUM ALBUMIN PRECURSOR.

The mouse gene of interest is Alb1 (albumin 1), ortholog of human ALB (albumin). Aliases include Alb-1, serum albumin variant.

ALB is a secreted blood serum protein expressed primarily in liver that functions as a stabilizer of extracellular fluid volume and as a carrier for hydrophobic compounds, such as steroids, fatty acids, thyroid hormones, and xenobiotics (OMIM 103600). ALB is also a potent antioxidant and is used for treatment or prevention of cerebral ischemic brain injury (Gum et al, Stroke 35(2):590-5 (2004)). Mutations in the ALB gene can give rise to familial dysalbuminemic hyperthyroxinemia. Under this disorder, ALB has a higher affinity for thyroxine (T4), which results in high total T4 but normal free T4 in serum. Consequently, familial dysalbuminemic hyperthyroxinemia can lead to misdiagnosis of hyperthyroidism and inappropriate treatment (Petitpas et al, Proc Natl Acad Sci USA 100(11):6440-5 (2003)). Individuals with analbuminemia, an autosomal recessive disorder where serum ALB is absent, appear to be healthy; however, this disorder can sometimes lead to atherosclerosis.

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 13 | 47 | 20 | 80 |
| Expected | 20 | 40 | 20 | 80 |

Chi-Sq.=6.35 Significance=0.041794106 (hom/n)=0.26 Avg. Litter Size=10

### Mutation Information

Mutation Type: Homologous Recombination (standard)
Description: Coding exons 1 through 5 were targeted (NCBI accession NM_{—}009654. 1).
1. Wild-type Expression Panel: Expression of the target gene was detected in all 13 adult tissue samples tested by RT-PCR, except brain and spinal cord.
2. QC Expression: Disruption of the target gene was confirmed by Southern hybridization analysis.

70.35.1. Phenotypic Analysis (for Disrupted Gene: DNA88062 (UNQ696)

(a) Overall Phenotypic Summary:

Mutation of the gene encoding the ortholog of human albumin (ALB) resulted in a notably decreased serum albumin in (−/−) mice. The female (−/−) mice also showed a decreased mean serum glucose level and the male (−/−) mice exhibited an increased mean serum cholesterol level. Decrease in insulin levels was also observed in the (−/−) mice. The female (−/−) mice were smaller with a decreased body length. The (−/−) mice exhibited an increased IL-6, MCP1 and TNF alpha response to LPS challenge. Disruption of the target gene was confirmed by Southern hybridization analysis.

(b) Immunology Phenotypic Analysis

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

T lymphocytes (T cells) are an important component of a mammalian immune response. T cells recognize antigens which are associated with a self-molecule encoded by genes within the major histocompatibility complex (MHC). The antigen may be displayed together with MHC molecules on the surface of antigen presenting cells, virus infected cells, cancer cells, grafts, etc. The T cell system eliminates these altered cells which pose a health threat to the host mammal. T cells include helper T cells and cytotoxic T cells. Helper T cells proliferate extensively following recognition of an antigen-MHC complex on an antigen presenting cell. Helper T cells also secrete a variety of cytokines, i.e., lymphokines, which play a central role in the activation of B cells, cytotoxic T cells and a variety of other cells which participate in the immune response.

In many immune responses, inflammatory cells infiltrate the site of injury or infection. The migrating cells may be neutrophilic, eosinophilic, monocytic or lymphocytic as can be determined by histologic examination of the affected tissues. Current Protocols in Immunology, ed. John E. Coligan, 1994, John Wiley & Sons, Inc.

Many immune related diseases are known and have been extensively studied. Such diseases include immune-mediated inflammatory diseases (such as rheumatoid arthritis, immune mediated renal disease, hepatobiliary diseases, inflammatory bowel disease (IBD), psoriasis, and asthma), non-immune-mediated inflammatory diseases, infectious diseases, immunodeficiency diseases, neoplasia, and graft rejection, etc. In the area of immunology, targets were identified for the treatment of inflammation and inflammatory disorders.

In the area of immunology, targets have been identified herein for the treatment of inflammation and inflammatory disorders. Immune related diseases, in one instance, could be treated by suppressing the immune response. Using neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

The following test was performed:

Acute Phase Response:

Test Description Bacterial lipopolysaccharide (LPS) is an endotoxin, and as such is a potent inducer of an acute phase response and systemic inflammation. The Level I LPS mice were injected intraperitoneally (i.p.) with a sublethal dose of LPS in 200 μL sterile saline using a 26 gauge needle. The doses were based on the average weight of the mice tested at 1 μg/g body weight 3 hours after injection; a 100 ul blood sample was then taken and analyzed for the presence of TNFa, MCP-1, and IL-6 on the FACSCalibur instrument.

Results:

The (−/−) mice exhibited an increased mean serum IL-6, MCP1 and TNF alpha response to LPS challenge when compared with their (+/+) littermates and the historical mean.

In summary, the LPS endotoxin challenge demonstrated that knockout mice deficient in the gene encoding PRO2155 polypeptides exhibit immunological abnormalities when compared with their wild-type littermates. In particular, the mutant mice exhibited an increased ability to elicit an immunological response (TNF-alpha, MCP-1 and IL-6 production) when challenged with the LPS endotoxin indicating a proinflammatory response. TNF-alpha, MCP-1 and IL-6 contribute to the later stages of B cell activation. TNF-alpha is an important inflammatory mediator. In addition, both TNF-alpha, MCP-1 and IL-6 play a critical role in inducing the acute phase response and systemic inflammation. TNF-alpha can substitute for the membrane-bound signal in macrophage activation (thus serving as an effector molecule). This suggests that inhibitors or antagonists to PRO2155 polypeptides would stimulate the immune system and would find utility in the cases wherein this effect would be beneficial to the individual such as in the case of leukemia, and other types of cancer, and in immunocompromised patients, such as AIDS sufferers. Accordingly, PRO2155 polypeptides or agonists thereof would be useful in inhibiting the immune response and would be useful candidates for suppressing harmful immune responses, e.g. in the case of graft rejection or graft-versus-host diseases.

(c) Phenotypic Analysis: Cardiology

In the area of cardiovascular biology, targets were identified herein for the treatment of hypertension, atherosclerosis, heart failure, stroke, various coronary artery diseases, dyslipidemias such as high cholesterol (hypercholesterolemia), diabetes and/or obesity. The phenotypic tests included the measurement of serum cholesterol.

Blood Lipids

Procedure: A cohort of 4 wild type, 4 heterozygous and 8 homozygous mice were tested in this assay. High cholesterol levels are recognized risk factors in the development of cardiovascular disease and/or diabetes. Measuring blood lipids facilitates the finding of biological switches that regulate blood lipid levels Inhibition of factors which elevate blood lipid levels may be useful for reducing the risk for cardiovascular disease. In these blood chemistry tests, measurements were recorded using the COBAS Integra 400 (mfr: Roche).

Results:

The male (−/−) mice exhibited an increased mean serum cholesterol level when compared with their gender-matched (+/+) littermates and the historical means.

As summarized above, the (−/−) mice exhibited notably increased cholesterol levels when compared with their gender-matched (+/+) littermates and the historical means. Thus, mutant mice deficient in the PRO2155 gene can serve as a model for cardiovascular disease. PRO2155 polypeptides or its encoding gene would be useful in regulating blood lipids such as cholesterol. Thus, PRO1255 polypeptides or agonists thereof would be useful in the treatment of such cardiovascular diseases as hypertension, atherosclerosis, heart failure, stroke, various coronary diseases, hypercholesterolemia, diabetes and/or obesity.

(d) Phenotypic Analysis: Metabolism-Blood Chemistry

In the area of metabolism, targets may be identified for the treatment of diabetes. Blood chemistry phenotypic analysis includes blood glucose measurements. In addition to measuring blood glucose levels the following blood chemistry tests are also routinely performed: Alkaline Phosphatase; Alanine Amino-Transferase; Albumin; Bilirubin; Phosphorous; Creatinine; BUN=Blood Urea Nitrogen; Calcium; Uric Acid; Sodium; Potassium; and Chloride. The COBAS Integra 400 (mfr: Roche) was used for running blood chemistry tests on the mice. In the area of metabolism, targets may be identified for the treatment of diabetes.

Results:

The (−/−) mice exhibited a notably decreased mean serum albumin level indicative of liver function problems when compared with their (+/+) littermates and the historical mean. The (−/−) mice also exhibited an increased mean serum alkaline phosphatase level and a decreased mean serum glucose level. However, decrease in insulin levels was observed in the (−/−) mice.

(e) Bone Metabolism & Body Diagnostics

(1) Tissue Mass & Lean Body Mass Measurements—Dexa

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in total tissue mass (TTM).

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI, i.e., whole body, vertebrae, and both femurs).

Body Measurements (Body Length & Weight):

Body Measurements: A measurement of body length and weight was performed at approximately 16 weeks of age.

Results:

The female (−/−) mice exhibited decreased mean body length when compared with their gender-matched (+/+) littermates and the historical mean. This observation would suggest some degree of growth retardation. However, other indices of abnormal growth were not observed.

70.36. Generation and Analysis of Mice Comprising DNA64886-1601 (UNQ705) Gene Disruptions

In these knockout experiments, the gene encoding PRO1356 polypeptides (designated as DNA64886-1601) (UNQ705) was disrupted. The gene specific information for these studies is as follows: the mutated mouse gene corresponds to nucleotide reference: NM_{—}016675 ACCESSION:NM_{—}016675 NID: gi 7710003 ref NM_{—}016675.1 Mus musculus claudin 2 (Cldn2); protein reference: 088552 ACCESSION:088552 NID: Mus musculus (Mouse). Claudin-2; the human gene sequence reference: NM_{—}020384 ACCESSION:NM_{—}020384 NID: gi 9966780 ref NM_{—}020384.1 Homo sapiens claudin 2 (CLDN2); the human protein sequence corresponds to reference: P57739 ACCESSION:P57739 NID: Homo sapiens (Human). Claudin-2.

The mouse gene of interest is Cldn2 (claudin 2), ortholog of human CLDN2.

CLDN2 is an integral plasma membrane protein, consisting of four transmembrane segments within a claudin family domain (Pfam accession PF00822). CLDN2 functions as a cell adhesion molecule that forms tight junctions in endothelial and epithelial cells. Tight junctions are important for regulating paracellular transport processes (Colegio et al, Am J Physiol Cell Physiol 284(6):1346-54 (2003); Gonzales-Mariscal et al, Prog Biophys Mol Biol 81(1):1-44 (2003); Tsukita et al, Nat Rev Mol Cell Bio12(4):285-93 (2001); Morita et al, Proc Natl Acad Sci USA 96(2):511-6 (1999)).

This mutation is in an X-linked gene. Both male and female wild-type mice were analyzed, whereas only male hemizygous mutant and female heterozygous mice were analyzed. The male hemizygous (wild-type) and hemizygous mutant mice are designated as (+/+) and (−/−), respectively.

### Summary of X-linked Gene Distribution by Sex and Genotype

(Only the agouti pups from the male chimeras are included.)

Summary of X-linked Gene Distributions for Sex by Genotype

| | | | |
|---|---|---|---|
| | Agouti F1 | | F1a |
| Progeny | (M chimera × wt) | Progeny | (F het × wt) |

| Sex | wt | het | Sex | wt | het | hemi |
|---|---|---|---|---|---|---|
| M | 10 | 0 | M | 26 | n/a | 18 |
| F | 0 | 15 | F | 16 | 34 | n/a |

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129 SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 19 | 10 | 27 | 56 |
| Expected | 14 | 28 | 14 | 56 |

Chi-Sq.=25.43 Significance=3.0056997E-6 (hom/n)=0.48 Avg. Litter Size=6

### Mutation Information

Mutation Type: Homologous Recombination (standard)
Description: Coding exon 1 was targeted (NCBI accession NM_{—}016675.3).
1. Wild-type Expression Panel: Expression of the target gene was detected in all 13 adult tissue samples tested by RT-PCR, except spleen, lung, skeletal muscle, and bone.
2. QC Expression: Disruption of the target gene was confirmed by Southern hybridization analysis.

70.36.1. Phenotypic Analysis (for Disrupted Gene: DNA64886-1601 (UNQ705)

(a) Overall Phenotypic Summary:

Mutation of the gene encoding the ortholog of human claudin 2 (CLDN2) resulted in impaired glucose tolerance and increased fasting serum glucose levels in (0/−) mice. This mutation is in an X-linked gene. Both male and female wild-type mice were analyzed, whereas only male hemizygous mutant and female heterozygous mice were analyzed. The male hemizygous (wild-type) and hemizygous mutant mice are designated as (+/+) and (−/−), respectively.

The hemizygous mutant mice exhibited impaired glucose tolerance and an increased mean fasting serum glucose level when compared with their wild-type littermates and the historical mean. Female knockouts (−/−) exhibited decreased exploratory behavior. The mutant knockouts exhibited decreased percentages of Peyer's patch B cells. Disruption of the target gene was confirmed by Southern hybridization analysis.

(b) Immunology Phenotypic Analysis

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

T lymphocytes (T cells) are an important component of a mammalian immune response. T cells recognize antigens which are associated with a self-molecule encoded by genes within the major histocompatibility complex (MHC). The antigen may be displayed together with MHC molecules on the surface of antigen presenting cells, virus infected cells, cancer cells, grafts, etc. The T cell system eliminates these altered cells which pose a health threat to the host mammal. T cells include helper T cells and cytotoxic T cells. Helper T cells proliferate extensively following recognition of an antigen-MHC complex on an antigen presenting cell. Helper T cells also secrete a variety of cytokines, i.e., lymphokines, which play a central role in the activation of B cells, cytotoxic T cells and a variety of other cells which participate in the immune response.

In many immune responses, inflammatory cells infiltrate the site of injury or infection. The migrating cells may be neutrophilic, eosinophilic, monocytic or lymphocytic as can be determined by histologic examination of the affected tissues. Current Protocols in Immunology, ed. John E. Coligan, 1994, John Wiley & Sons, Inc.

Many immune related diseases are known and have been extensively studied. Such diseases include immune-mediated inflammatory diseases (such as rheumatoid arthritis, immune mediated renal disease, hepatobiliary diseases, inflammatory bowel disease (IBD), psoriasis, and asthma), non-immune-mediated inflammatory diseases, infectious diseases, immunodeficiency diseases, neoplasia, and graft rejection, etc. In the area of immunology, targets were identified for the treatment of inflammation and inflammatory disorders.

In the area of immunology, targets have been identified herein for the treatment of inflammation and inflammatory disorders. Immune related diseases, in one instance, could be treated by suppressing the immune response. Using neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

The following test was performed:

Flourescence-activated Cell-sorting (FACS) Analysis

Procedure:

FACS analysis of immune cell composition from peripheral blood was performed including CD4, CD8 and T cell receptor to evaluate T lymphocytes, CD19 for B lymphocytes, CD45 as a leukocyte marker and pan NK for natural killer cells. The FACS analysis was carried out on 2 wild type and 6 homozygous mice and included cells derived from thymus, spleen, bone marrow and lymph node.

In these studies, analyzed cells were isolated from thymus, peripheral blood, spleen, bone marrow and lymph nodes. Flow cytometry was designed to determine the relative proportions of CD4 and CD8 positive T cells, B cells, NK cells and monocytes in the mononuclear cell population. A Becton-Dickinson FACSCalibur 3-laser FACS machine was used to assess immune status. For Phenotypic Assays and Screening, this machine records CD4+/CD8−, CD8+/CD4−, NK, B cell and monocyte numbers in addition to the CD4+/CD8+ ratio. The mononuclear cell profile was derived by staining a single sample of lysed peripheral blood from each mouse with a panel of six lineage-specific antibodies: CD45 PerCP, anti-TCRb APC, CD4 PE, CD8 FITC, pan-NK PE, and CD19 FITC. The two FITC and PE labeled antibodies stain mutually exclusive cell types. The samples were analyzed using a Becton Dickinson FACSCalibur flow cytometer with CellQuest software.

Results:

FACS: The homozygous (−/−) mice exhibited decreased percentage of B cells in Peyer's patches compared to their gender-matched wildtype (+/+) littermates and the historical means. Peyer's patches are aggregates of lymphocytes along the small intestine, especially the ileum.

In summary, FACS analysis of immune cell composition indicates that knockout mice (−/−) exhibit immunological differences with respect to B cells compared with their wildtype (+/+) littermates. Thus, PRO1356 polypeptides or agonists thereof would be useful in B cell production, whereas antagonists or inhibitors of PRO1356 polypeptides would be expected to lead to the opposite effects.

(c) Phenotypic Analysis: Metabolism-Blood Chemistry/Glucose Tolerance

In the area of metabolism, targets may be identified for the treatment of diabetes. Blood chemistry phenotypic analysis includes blood glucose measurements. The COBAS Integra 400 (mfr: Roche) was used for running blood chemistry tests on the mice. In the area of metabolism, targets may be identified for the treatment of diabetes. Blood chemistry phenotypic analysis includes glucose tolerance tests to measure insulin sensitivity and changes in glucose metabolism. Abnormal glucose tolerance test results may indicate but may not be limited to the following disorders or conditions: Diabetes Type 1 and Type 2, Syndrome X, various cardiovascular diseases and/or obesity.

Procedure: A cohort of 2 wild type and 4 homozygous mice were used in this assay. The glucose tolerance test is the standard for defining impaired glucose homeostasis in mammals. Glucose tolerance tests were performed using a Lifescan glucometer. Animals were injected IP at 2 g/kg with D-glucose delivered as a 20% solution and blood glucose levels were measured at 0, 30, 60 and 90 minutes after injection.

Results:

Blood Glucose Levels/Glucose Tolerance Test:

The (0/−) mice exhibited impaired glucose tolerance when compared with their gender-matched (+/+) littermates and the historical means. The (0/−) mice also exhibited an increased mean fasting serum glucose level.

These studies indicated that (0/−) mice exhibit a decreased or impaired glucose tolerance in the presence of normal fasting glucose at all 3 intervals tested when compared with their gender-matched (+/+) littermates and the historical means. Thus, knockout mutant mice exhibited the phenotypic pattern of an impaired glucose homeostasis, and therefor PRO1356 polypeptides (or agonists thereof) or its encoding gene would be useful in the treatment of conditions associated with an impaired glucose homeostasis and/or various cardiovascular diseases, including diabetes.

(d) Phenotypic Analysis: CNS/Neurology

In the area of neurology, analysis focused herein on identifying in vivo validated targets for the treatment of neurological and psychiatric disorders including depression, generalized anxiety disorders, attention deficit hyperactivity disorder, obsessive compulsive disorder, schizophrenia, cognitive disorders, hyperalgesia and sensory disorders. Neurological disorders include the category defined as “anxiety disorders” which include but are not limited to: mild to moderate anxiety, anxiety disorder due to a general medical condition, anxiety disorder not otherwise specified, generalized anxiety disorder, panic attack, panic disorder with agoraphobia, panic disorder without agoraphobia, posttraumatic stress disorder, social phobia, specific phobia, substance-induced anxiety disorder, acute alcohol withdrawal, obsessive compulsive disorder, agoraphobia, bipolar disorder I or II, bipolar disorder not otherwise specified, cyclothymic disorder, depressive disorder, major depressive disorder, mood disorder, substance-induced mood disorder. In addition, anxiety disorders may apply to personality disorders including but not limited to the following types: paranoid, antisocial, avoidant behavior, borderline personality disorders, dependent, histronic, narcissistic, obsessive-compulsive, schizoid, and schizotypal.

Procedure:

Behavioral screens were performed on a cohort of 4 wild type, 4 heterozygous and 8 homozygous mice. All behavioral tests were done between 12 and 16 weeks of age unless reduced viability necessitates earlier testing. These tests included open field to measure anxiety, activity levels and exploration.

Functional Observational Battery (FOB) Test

The FOB is a series of situations applied to the animal to determine gross sensory and motor deficits. A subset of tests from the Irwin neurological screen that evaluates gross neurological function is used. In general, short-duration, tactile, olfactory, and visual stimuli are applied to the animal to determine their ability to detect and respond normally. These simple tests take approximately 10 minutes and the mouse is returned to its home cage at the end of testing.

Open Field Test:

Several targets of known drugs have exhibited phenotypes in the open field test. These include knockouts of the seratonin transporter, the dopamine transporter (Giros et al., Nature. 1996 Feb. 15; 379(6566):606-12), and the GABA receptor (Homanics et al., Proc Natl Acad Sci USA. 1997 Apr. 15; 94(8):4143-8). An automated open-field assay was customized to address changes related to affective state and exploratory patterns related to learning. First, the field (40×40 cm) was selected to be relatively large for a mouse, thus designed to pick up changes in locomotor activity associated with exploration. In addition, there were 4 holes in the floor to allow for nose-poking, an activity specifically related to exploration. Several factors were also designed to heighten the affective state associated with this test. The open-field test is the first experimental procedure in which the mice are tested, and the measurements that were taken were the subjects' first experience with the chamber. In addition, the open-field was brightly lit. All these factors will heighten the natural anxiety associated with novel and open spaces. The pattern and extent of exploratory activity, and especially the center-to-total distance traveled ratio, may then be able to discern changes related to susceptibility to anxiety or depression. A large arena (40 cm×40 cm, VersaMax animal activity monitoring system from AccuScan Instruments) with infrared beams at three different levels was used to record rearing, hole poke, and locomotor activity. The animal was placed in the center and its activity was measured for 20 minutes. Data from this test was analyzed in five, 4-minute intervals. The total distance traveled (cm), vertical movement number (rearing), number of hole pokes, and the center to total distance ratio were recorded.

The propensity for mice to exhibit normal habituation responses to a novel environment is assessed by determining the overall change in their horizontal locomotor activity across the 5 time intervals. This calculated slope of the change in activity over time is determined using normalized, rather than absolute, total distance traveled. The slope is determined from the regression line through the normalized activity at each of the 5 time intervals. Normal habituation is represented by a negative slope value.

Results:

General & Exploratory Activity: The (−/−) mice exhibited decreased rearing activity and hole poking when compared with their (+/+) littermates, suggesting a decreased exploratory response in the mutants.
Open Field Testing: The (−/−) mice exhibited a decreased exploratory response when compared with their gender-matched (+/+) littermates, which is indicative of a decreased anxiety-like response in the mutants. Thus, knockout mice demonstrated a phenotype consistent with depression, generalized anxiety disorders, cognitive disorders, hyperalgesia and sensory disorders and/or bipolar disorders. Thus, PRO1356 polypeptides and agonists thereof would be useful for the treatment or amelioration of the symptoms associated with depressive disorders.

70.37. Generation and Analysis of Mice Comprising DNA68869-1610 (UNQ720) Gene Disruptions

In these knockout experiments, the gene encoding PRO1385 polypeptides (designated as DNA68869-1610) (UNQ720) was disrupted. The gene specific information for these studies is as follows: the mutated mouse gene corresponds to nucleotide reference: NM_{—}178780 Mus musculus RIKEN cDNA E130307J04 gene (E130307J04Rik); protein reference: NP_{—}848895 RIKEN cDNA E130307J04 gene [Mus musculus]; the human gene sequence reference: NM_{—}024557 Homo sapiens RIC3 protein (RIC3); the human protein sequence corresponds to reference: NP_{—}078833 RIC3 protein [Homo sapiens].

The targeted mouse gene encodes a hypothetical protein (RIKEN cDNA E130307J04), which is the ortholog of human RIC3 (RIC3 protein). Aliases include RIC-3, hric3, and FLJ11608.

RIC3 is most likely a membrane protein that belongs to a conserved family of genes thought to regulate nAChR-mediated transmission throughout evolution (PMID: 12821669). Targeted or gene trap mutations are generated in strain 129SvEv^{Brd}-derived embryonic stem (ES) cells.

The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 17 | 29 | 20 | 66 |
| Expected | 16.5 | 33 | 16.5 | 66 |

Chi-Sq.=1.99 Significance=0.36972344 (hom/n)=0.23 Avg. Litter Size=8

### Mutation Information

Mutation Type: Homologous Recombination (standard)
Description: Coding exon 1 was targeted (Accession: NM_{—}178780).
1. Wild-type Expression Panel: Expression of the target gene was detected in embryonic stem (ES) cells and in all 13 adult tissue samples tested by RT-PCR, except lung, liver, skeletal muscle, bone, and adipose.
2. QC Expression: Disruption of the target gene was confirmed by Southern hybridization analysis.

### 70.37.1. Phenotypic Analysis (for Disrupted Gene: DNA68869-1610 (UNQ720)

(a) Overall Phenotypic Summary:

Mutation of the gene encoding the ortholog of human RIC3 resulted in an enhanced glucose tolerance in the (−/−) mice. Male homozygous mice showed a decreased heart rate. The male (−/−) mice exhibited an increased femoral bone measurements. Gene disruption was confirmed by Southern blot.

(b) Phenotypic Analysis: Metabolism-Blood Chemistry/Glucose Tolerance

In the area of metabolism, targets may be identified for the treatment of diabetes. Blood chemistry phenotypic analysis includes blood glucose measurements. The COBAS Integra 400 (mfr: Roche) was used for running blood chemistry tests on the mice. In addition to measuring blood glucose levels the following blood chemistry tests are also routinely performed: Alkaline Phosphatase; Alanine Amino-Transferase; Albumin; Bilirubin; Phosphorous; Creatinine; BUN=Blood Urea Nitrogen; Calcium; Uric Acid; Sodium; Potassium; and Chloride. In the area of metabolism, targets may be identified for the treatment of diabetes. Blood chemistry phenotypic analysis includes glucose tolerance tests to measure insulin sensitivity and changes in glucose metabolism. Abnormal glucose tolerance test results may indicate but may not be limited to the following disorders or conditions: Diabetes Type 1 and Type 2, Syndrome X, various cardiovascular diseases and/or obesity.

Procedure: A cohort of 2 wild type and 4 homozygous mice were used in this assay. The glucose tolerance test is the standard for defining impaired glucose homeostasis in mammals. Glucose tolerance tests were performed using a Lifescan glucometer. Animals were injected IP at 2 g/kg with D-glucose delivered as a 20% solution and blood glucose levels were measured at 0, 30, 60 and 90 minutes after injection.

Results:

Glucose Tolerance Test: The male (−/−) mice exhibited enhanced glucose tolerance when compared with their gender-matched (+/+) littermates; however, the glucose levels of the male (−/−) mice were still within the historical normal range at the 3 measured intervals.

(c) Diagnostics—Blood Pressure

Description:

Systolic blood pressure is measured via a noninvasive tail-cuff method for four days on the Visitech BP-2000 Blood Pressure Analysis System. The blood pressure is measured ten times each day for four days. The four days are then averaged to obtain a mouse's conscious systolic blood pressure.

Results:

The (−/−) mice exhibited a decreased mean systolic blood pressure (two standard deviations lower) when compared with their (+/+) littermates and the historical mean.

(d) Bone Metabolism & Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body len/gth and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Bone MicroCT Analysis:

Procedure: MicroCT was also used to get very sensitive measurements of BMD. One vertebra and 1 femur were taken from a cohort of 4 wild type and 8 homozygous mice. Measurements were taken of lumbar 5 vertebra trabecular bone volume, trabecular thickness, connectivity density and midshaft femur total bone area and cortical thickness. The μCT40 scans provided detailed information on bone mass and architecture. Multiple bones were placed into sample holders and scanned automatically. Instrument software was used to select regions of interest for analysis. Trabecular bone parameters were analyzed in the fifth lumbar vertebrae (LV5) at 16 micrometer resolution and cortical bone parameters were analyzed in the femur midshaft at a resolution of 20 micrometers.

Results:

Micro-CT: The male (−/−) mice exhibited increased mean femoral mid-shaft cortical thickness when compared with their gender-matched (+/+) littermates and the historical means.

In summary, the (−/−) mice exhibited increased femoral mid-shaft cross-sectional thickness when compared with their gender-matched (+/+) littermates. These results indicate that the knockout mutant phenotype is associated with such bone abnormalities as osteopetrosis. Osteopetrosis is a condition characterized by abnormal thickening and hardening of bone and abnormal fragility of the bones. As such, PRO1385 polypeptides or agonists thereof would be beneficial for the treatment of osteopetrosis or other osteo-related diseases. On the other hand, inhibitors or antagonists of PRO1385 polypeptides would be useful in bone healing.

70.38. Generation and Analysis of Mice Comprising DNA64897-1628 (UNQ730) Gene Disruptions

In these knockout experiments, the gene encoding PRO1412 polypeptides (designated as DNA64897-1628) (UNQ730) was disrupted. The gene specific information for these studies is as follows: the mutated mouse gene corresponds to nucleotide reference: NM_{—}028732 Mus musculus RIKEN cDNA 4632428N05 gene (4632428N05Rik); protein reference: Q9D659 ACCESSION:Q9D659 NID: Mus musculus (Mouse). Mus musculus 0 day neonate skin cDNA, RIKEN full-length enriched library, clone:4632428N05 product:hypothetical Immunoglobulin and major histocompatibility complex domain/Immunoglobulin subtype containing protein, full insert sequence (RIKEN cDNA 4632428N05); the human gene sequence reference: AY358379 Homo sapiens clone DNA64897 GVPT730 (UNQ730); the human protein sequence corresponds to reference: Q6UXF3 ACCESSION:Q6UXF3 NID: Homo sapiens (Human). GVPT730.

The mouse gene of interest is RIKEN cDNA 4632428N05 gene, ortholog of human PP2135 protein. Aliases include GI24, FLJ00041, and platelet receptor GI24.

PP2135 protein is a hypothetical type I plasma membrane protein, containing a signal sequence, an immunoglobulin-like domain (Pfam accession PF00047), a transmembrane segment, and a short C-terminal segment. Immunoglobulin-like domains are usually involved in protein-protein interactions and are found in a wide variety of proteins. Thus, PP2135 protein is likely to function as a receptor or cell adhesion molecule.

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 23 | 44 | 18 | 85 |
| Expected | 21.25 | 42.5 | 21.25 | 85 |

Chi-Sq.=0.4 Significance=0.8187308 (hom/n)=0.24 Avg. Litter Size=10

### Mutation Information

Mutation Type: Homologous Recombination (standard)
Description: Coding exon 1 was targeted (NCBI accession NM_{—}028732.2).
1. Wild-type Expression Panel: Expression of the target gene was detected in embryonic stem (ES) cells and in all 13 adult tissue samples tested by RT-PCR, except heart.
2. QC Expression: Disruption of the target gene was confirmed by Southern hybridization analysis.

70.38.1. Phenotypic Analysis (for Disrupted Gene: DNA64897-1628 (UNQ730)

(a) Overall Phenotypic Summary:

Mutation of the gene encoding the ortholog of a human hypothetical type I plasma membrane protein (PP2135) resulted in immunological abnormalities in (−/−) mice. Male (−/−) mice exhibited enhanced glucose tolerance. The homozygous mutant mice exhibited increased mean white blood cell and absolute neutrophil counts and an increased mean percentage of CD4 cells in the peripheral blood when compared with their wild-type littermates and the historical means. Disruption of the target gene was confirmed by Southern hybridization analysis.

(b) Phenotypic Analysis: Metabolism-Blood Chemistry/Glucose Tolerance

In the area of metabolism, targets may be identified for the treatment of diabetes. Blood chemistry phenotypic analysis includes blood glucose measurements. The COBAS Integra 400 (mfr: Roche) was used for running blood chemistry tests on the mice. In the area of metabolism, targets may be identified for the treatment of diabetes. Blood chemistry phenotypic analysis includes glucose tolerance tests to measure insulin sensitivity and changes in glucose metabolism. Abnormal glucose tolerance test results may indicate but may not be limited to the following disorders or conditions: Diabetes Type 1 and Type 2, Syndrome X, various cardiovascular diseases and/or obesity.

Procedure: A cohort of 2 wild type and 4 homozygous mice were used in this assay. The glucose tolerance test is the standard for defining impaired glucose homeostasis in mammals. Glucose tolerance tests were performed using a Lifescan glucometer. Animals were injected IP at 2 g/kg with D-glucose delivered as a 20% solution and blood glucose levels were measured at 0, 30, 60 and 90 minutes after injection.

Results:

Glucose Tolerance Test: The male mutant (−/−) mice tested exhibited enhanced glucose tolerance when compared with their gender-matched (+/+) littermates and the historical means.

In these studies the mutant (−/−) mice showed an increased or enhanced glucose tolerance in the presence of normal fasting glucose at all 3 intervals tested when compared with their gender-matched (+/+) littermates and the historical means. In addition, hyperinsulinemia was not apparent in the (−/−) mice. Thus, knockout mice exhibited an increased insulin sensitivity or the opposite phenotypic pattern of an impaired glucose homeostasis, and as such antagonists (inhibitors) to PRO1412 polypeptides or its encoding gene would be useful in the treatment of impaired glucose homeostasis.

(c) Immunology Phenotypic Analysis

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

T lymphocytes (T cells) are an important component of a mammalian immune response. T cells recognize antigens which are associated with a self-molecule encoded by genes within the major histocompatibility complex (MHC). The antigen may be displayed together with MHC molecules on the surface of antigen presenting cells, virus infected cells, cancer cells, grafts, etc. The T cell system eliminates these altered cells which pose a health threat to the host mammal. T cells include helper T cells and cytotoxic T cells. Helper T cells proliferate extensively following recognition of an antigen-MHC complex on an antigen presenting cell. Helper T cells also secrete a variety of cytokines, i.e., lymphokines, which play a central role in the activation of B cells, cytotoxic T cells and a variety of other cells which participate in the immune response.

In many immune responses, inflammatory cells infiltrate the site of injury or infection. The migrating cells may be neutrophilic, eosinophilic, monocytic or lymphocytic as can be determined by histologic examination of the affected tissues. Current Protocols in Immunology, ed. John E. Coligan, 1994, John Wiley & Sons, Inc.

Many immune related diseases are known and have been extensively studied. Such diseases include immune-mediated inflammatory diseases (such as rheumatoid arthritis, immune mediated renal disease, hepatobiliary diseases, inflammatory bowel disease (IBD), psoriasis, and asthma), non-immune-mediated inflammatory diseases, infectious diseases, immunodeficiency diseases, neoplasia, and graft rejection, etc. In the area of immunology, targets were identified for the treatment of inflammation and inflammatory disorders.

In the area of immunology, targets have been identified herein for the treatment of inflammation and inflammatory disorders. Immune related diseases, in one instance, could be treated by suppressing the immune response. Using neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

The following test was performed:

Hematology Analysis:

Test Description: Blood Tests are Carried out by Abbott's Cell-Dyn 3500R, an automated hematology analyzer. Some of its features include a five-part WBC differential. ‘Patient’ reports can cover over 22 parameters in all.

Results:

The (−/−) mice exhibited increased mean total white blood cell and absolute neutrophil counts when compared with their (+/+) littermates and the historical mean.

In summary, the hematology results indicate that the homozygous mutant mice exhibited an increased absolute neutrophil count compared to their (+/+) littermate controls indicating elevated levels of precursors of macrophages. These results indicate that the homozygous (−/−) knockout mice exhibit an abnormal immunological phenotype.

Flourescence-activated Cell-sorting (FACS) Analysis

Procedure:

FACS analysis of immune cell composition from peripheral blood was performed including CD4, CD8 and T cell receptor to evaluate T lymphocytes, CD19 for B lymphocytes, CD45 as a leukocyte marker and pan NK for natural killer cells. The FACS analysis was carried out on 2 wild type and 6 homozygous mice and included cells derived from thymus, spleen, bone marrow and lymph node.

In these studies, analyzed cells were isolated from thymus, peripheral blood, spleen, bone marrow and lymph nodes. Flow cytometry was designed to determine the relative proportions of CD4 and CD8 positive T cells, B cells, NK cells and monocytes in the mononuclear cell population. A Becton-Dickinson FACSCalibur 3-laser FACS machine was used to assess immune status. For Phenotypic Assays and Screening, this machine records CD4+/CD8−, CD8+/CD4−, NK, B cell and monocyte numbers in addition to the CD4+/CD8+ratio. The mononuclear cell profile was derived by staining a single sample of lysed peripheral blood from each mouse with a panel of six lineage-specific antibodies: CD45 PerCP, anti-TCRb APC, CD4 PE, CD8 FITC, pan-NK PE, and CD19 FITC. The two FITC and PE labeled antibodies stain mutually exclusive cell types. The samples were analyzed using a Becton Dickinson FACSCalibur flow cytometer with CellQuest software.

Results:

FACS: The (−/−) mice exhibited an increased mean percentage of CD4 and CD8 cells and lower percentage of B cells when compared with their (+/+) littermates and the historical mean. However, the (−/−) mice exhibited less CD11 blow cells in the peritoneum. Thus, knocking out the gene which encodes PRO1412 polypeptides causes an increase in the T cell population. From these observations, PRO1412 polypeptides or the gene encoding PRO1412 appears to act as a negative regulator of T cell proliferation. Thus, PRO1412 polypeptides or agonists thereof would be beneficial as a negative regulator of T cell proliferation in those instances wherein a pronounced T-cell proliferation is present such as occurs in autoimmune diseases (for example rheumatoid arthritis patients). In addition, PRO1412 polypeptides would be especially useful in preventing skin graft rejections.

70.39. Generation and Analysis of Mice Comprising DNA68836-1656 (UN0756) Gene Disruptions

In these knockout experiments, the gene encoding PRO1487 polypeptides (designated as DNA68836-1656) (UNQ756) was disrupted. The gene specific information for these studies is as follows: the mutated mouse gene corresponds to nucleotide reference: XM_{—}194358 Mus musculus similar to mKIAA0990 protein (LOC269941); protein reference: Q6ZQ11 ACCESSION:Q6ZQ11 NID: Mus musculus (Mouse). MKIAA0990 protein (Fragment); the human gene sequence reference: NM_{—}014918 Homo sapiens carbohydrate (chondroitin) synthase 1 (CHSY1); the human protein sequence corresponds to reference: Q9Y2J5 ACCESSION:Q9Y2J5 NID: Homo sapiens (Human). Hypothetical protein KIAA0990 (Chondroitin synthase).

The mouse gene of interest is Chsy1 (carbohydrate [chondroitin] synthase 1), ortholog of human CHSY1. Aliases include KIAA0990, mKIAA0990, KIAA0990, and chondroitin synthase.

CHSY1 is a type II membrane protein in the Golgi apparatus that catalyzes the synthesis of chondroitin, a polysaccharide polymer of chondroitin sulfate proteoglycans. CHSY1 possesses both beta-1,3-glucuronic acid transferase and beta-1,4-N-acetylgalactosamine transferase activities. For chondroitin polymerization to occur, CHSY1 requires coexpression of chondroitin polymerizing factor, which serves as a specific activating factor. CHSY1 is an enzyme that plays a central role in the biosynthesis of chondroitin sulfate proteoglycans. These proteoglycans are located on the surface of cells and in the extracellular matrix and function as mediators of axon growth, pathfinding, and neural network formation (Kitagawa et al, J Biol Chem 276(42):38721-6 (2001); Kitagawa et al, J Biol Chem 278(26):23666-71 (2003); Sandvig et al, Glia 46(3):225-51 (2004)).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 24 | 30 | 18 | 72 |
| Expected | 18 | 36 | 18 | 72 |

Chi-Sq.=0.5 Significance=0.7788008 (hom/n)=0.23 Avg. Litter Size=8

### Mutation Information

Mutation Type: Homologous Recombination (standard)
Description: Coding exon 1 was targeted (NCBI accession AK129255. 1).
1. Wild-type Expression Panel: Expression of the target gene was detected in embryonic stem (ES) cells and in all 13 adult tissue samples tested by RT-PCR, except adipose.
2. QC Expression: Disruption of the target gene was confirmed by Southern hybridization analysis.

70.39.1. Phenotypic Analysis (for Disrupted Gene: DNA68836-1656 (UN0756)

(a) Overall Phenotypic Summary:

Mutation of the gene encoding the ortholog of human carbohydrate (chondroitin) synthase 1 (CHSY1) resulted in arthritis, retinal degeneration, impaired motor strength, and decreased bone mineral measurements in (−/−) mice. The (−/−) mice also exhibited abnormalities of the red blood cells. The homozygous mutant mice exhibited chronic-active arthritis and notably decreased bone mineral measurements when compared with their wild-type littermates. Disruption of the target gene was confirmed by Southern hybridization analysis.

(b) Pathology:

### Microscopic Observations:

The (−/−) mice exhibited chronic-active arthritis with proliferative chondropathy and an arthropathy involving the femur tibial joint and diffuse mild retinal degeneration. The lesions in the femur tibial joint included proliferation of cartilage and chondrous metaplasia of cruciate ligaments and perichondral connective tissues. Chronic active inflammation was present in periarticular connective tissues and extended into adjacent skeletal muscle.

The retinal degeneration noted in the (−/−) mice was characterized by a diffuse mild thinning of the external nuclear layer. Gene Expression: LacZ activity was not detected in the panel of tissues by immunohisto chemical analysis.

(c) Immunology Phenotypic Analysis

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

T lymphocytes (T cells) are an important component of a mammalian immune response. T cells recognize antigens which are associated with a self-molecule encoded by genes within the major histocompatibility complex (MHC). The antigen may be displayed together with MHC molecules on the surface of antigen presenting cells, virus infected cells, cancer cells, grafts, etc. The T cell system eliminates these altered cells which pose a health threat to the host mammal. T cells include helper T cells and cytotoxic T cells. Helper T cells proliferate extensively following recognition of an antigen-MHC complex on an antigen presenting cell. Helper T cells also secrete a variety of cytokines, i.e., lymphokines, which play a central role in the activation of B cells, cytotoxic T cells and a variety of other cells which participate in the immune response.

In many immune responses, inflammatory cells infiltrate the site of injury or infection. The migrating cells may be neutrophilic, eosinophilic, monocytic or lymphocytic as can be determined by histologic examination of the affected tissues. Current Protocols in Immunology, ed. John E. Coligan, 1994, John Wiley & Sons, Inc.

Many immune related diseases are known and have been extensively studied. Such diseases include immune-mediated inflammatory diseases (such as rheumatoid arthritis, immune mediated renal disease, hepatobiliary diseases, inflammatory bowel disease (IBD), psoriasis, and asthma), non-immune-mediated inflammatory diseases, infectious diseases, immunodeficiency diseases, neoplasia, and graft rejection, etc. In the area of immunology, targets were identified for the treatment of inflammation and inflammatory disorders.

In the area of immunology, targets have been identified herein for the treatment of inflammation and inflammatory disorders. Immune related diseases, in one instance, could be treated by suppressing the immune response. Using neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

The following test was performed:

Hematology Analysis:

Test Description Blood tests are carried out by Abbott's Cell-Dyn 3500R, an automated hematology analyzer. Some of its features include a five-part WBC differential. ‘Patient’ reports can cover over 22 parameters in all.

Results:

The (−/−) mice exhibited an increased mean corpuscular volume and an increased mean corpuscular hemoglobin level when compared with their (+/+) littermates and the historical means. The (−/−) mice also exhibited a decreased mean red blood cell distribution width. These results show that inhibitors or antagonists to PRO1487 polypeptides would mimic this abnormal phenotype.

(d) Cardiovascular Phenotypic Analysis:

In the area of cardiovascular biology, phenotypic testing was performed to identify potential targets for the treatment of cardiovascular, endothelial or angiogenic disorders. One such phenotypic test included optic fundus photography and angiography to determine the retinal arteriovenous ratio (A/V ratio) in order to flag various eye abnormalities. An abnormal A/V ratio signals such systemic diseases or disorders that may be related to the vascular disease of hypertension (and any disease that causes hypertension, e.g. atherosclerosis), diabetes or other ocular diseases corresponding to ophthalmological disorders. Such eye abnormalities may include but are not limited to the following: retinal abnormality is retinal dysplasia, various retinopathies, restenosis, retinal artery obstruction or occlusion; retinal degeneration causing secondary atrophy of the retinal vasculature, retinitis pigmentosa, macular dystrophies, Stargardt's disease, congenital stationary night blindness, choroideremia, gyrate atrophy, Leber's congenital amaurosis, retinoschisis disorders, Wagner's syndrome, Usher syndromes, Zellweger syndrome, Saldino-Mainzer syndrome, Senior-Loken syndrome, Bardet-Biedl syndrome, Alport's syndrome, Alstom's syndrome, Cockayne's syndrome, dysplaisa spondyloepiphysaria congentia, Flynn-Aird syndrome, Friedreich ataxia, Hallgren syndrome, Marshall syndrome, Albers-Schnoberg disease, Refsum's disease, Kearns-Sayre syndrome, Waardenburg's syndrome, Alagile syndrome, myotonic dystrophy, olivopontocerebellar atrophy, Pierre-Marie dunsdrome, Stickler syndrome, carotinemeia, cystinosis, Wolfram syndrome, Bassen-Kornzweig syndrome, abetalipoproteinemia, incontinentia pigmenti, Batten's disease, mucopolysaccharidoses, homocystinuria, or mannosidosis.

Procedure: A cohort of 4 wild type, 4 heterozygous and 8 homozygous mice were tested in this assay. Optic fundus photography was performed on conscious animals using a Kowa Genesis small animal fundus camera modified according to Hawes and coauthors (Hawes et al., 1999 Molecular Vision 1999; 5:22). Intra-peritoneal injection of fluorescein permitted the acquisition of direct light fundus images and fluorescent angiograms for each examination. In addition to direct ophthalmological changes, this test can detect retinal changes associated with systemic diseases such as diabetes and atherosclerosis or other retinal abnormalities. Pictures were provided of the optic fundus under normal light. The angiographic pictures allowed examination of the arteries and veins of the eye. In addition an artery to vein (A/V) ratio was determined for the eye.

Ophthalmology analysis was performed on generated F2 wild type, heterozygous, and homozygous progeny using the protocol described above. Specifically, the A/V ratio was measured and calculated according to the fundus images with Kowa COMIT+ software. This test takes color photographs through a dilated pupil: the images help in detecting and classifying many diseases. The artery to vein ratio (A/V) is the ratio of the artery diameter to the vein diameter (measured before the bifurcation of the vessels). Many diseases will influence the ratio, i.e., diabetes, cardiovascular disorders, papilledema, optic atrophy or other eye abnormalities such as retinal degeneration (known as retinitis pigmentosa) or retinal dysplasia, vision problems or blindness. Thus, phenotypic observations which result in an increased artery-to-vein ratio in homozygous (−/−) and heterozygous (+/−) mutant progeny compared to wildtype (+/+) littermates would be indicative of such pathological conditions.

Results:

Fundus: All 8 (−/−) mice exhibited multiple retinal degeneration spots with attenuated retinal arteries. The optic discs of the (−/−) mice were dilated, and the optic nerve heads were thinner than those of their (+/+) littermates.

In summary, in this study, (−/−) mice showed opthamological abnormalities which would lead to attentuated retinal vessels and retinal degeneration when compared with their (+/+) littermates. In summary, by knocking out the gene identified as DNA68836-1656 encoding PRO1487 polypeptides, homozygous mutant progeny exhibit phenotypes which are associated with retinal artery abnormalities. Such detected retinal changes are most commonly associated with cardiovascular systemic diseases or disorders that may be related to the vascular disease of hypertension (and any disease that causes hypertension, e.g. atherosclerosis), diabetes or other ocular diseases corresponding to ophthalmological disorders such as retinal degeneration. Thus, antagonists of PRO1487 encoding genes would lead to similar pathological retinal changes, whereas agonists would be useful as therapeutic agents in the treatment of hypertension, atherosclerosis or other opthamological disorders including retinal degeneration and diseases associated with this condition (as indicated above).

(e) Adult Skin Cell Proliferation:

Procedure: Skin cells were isolated from 16 week old animals (2 wild type and 4 homozygous mice). These were developed into primary fibroblast cultures and the fibroblast proliferation rates were measured in a strictly controlled protocol. The ability of this assay to detect hyper-proliferative and hypo-proliferative phenotypes has been demonstrated with p53 and Ku80. Proliferation was measured using Brdu incorporation.

Specifically, in these studies the skin fibroblast proliferation assay was used. An increase in the number of cells in a standardized culture was used as a measure of relative proliferative capacity. Primary fibroblasts were established from skin biopsies taken from wild type and mutant mice. Duplicate or triplicate cultures of 0.05 million cells were plated and allowed to grow for six days. At the end of the culture period, the number of cells present in the culture was determined using a electronic particle counter.

Results:

One female (−/−) mouse exhibited an increased mean skin fibroblast proliferation rate when compared with their gender-matched (+/+) littermates.

Thus, one homozygous mutant mouse demonstrated a hyper-proliferative phenotype. As suggested by these observations, PRO1487 polypeptides or agonists thereof could function as tumor suppressors and would be useful in decreasing abnormal cell proliferation.

(f) Phenotypic Analysis: CNS/Neurology

In the area of neurology, analysis focused herein on identifying in vivo validated targets for the treatment of neurological and psychiatric disorders including depression, generalized anxiety disorders, attention deficit hyperactivity disorder, obsessive compulsive disorder, schizophrenia, cognitive disorders, hyperalgesia and sensory disorders. Neurological disorders include the category defined as “anxiety disorders” which include but are not limited to: mild to moderate anxiety, anxiety disorder due to a general medical condition, anxiety disorder not otherwise specified, generalized anxiety disorder, panic attack, panic disorder with agoraphobia, panic disorder without agoraphobia, posttraumatic stress disorder, social phobia, specific phobia, substance-induced anxiety disorder, acute alcohol withdrawal, obsessive compulsive disorder, agoraphobia, bipolar disorder I or II, bipolar disorder not otherwise specified, cyclothymic disorder, depressive disorder, major depressive disorder, mood disorder, substance-induced mood disorder. In addition, anxiety disorders may apply to personality disorders including but not limited to the following types: paranoid, antisocial, avoidant behavior, borderline personality disorders, dependent, histronic, narcissistic, obsessive-compulsive, schizoid, and schizotypal.

Procedure:

Behavioral screens were performed on a cohort of 4 wild type, 4 heterozygous and 8 homozygous mice. All behavioral tests were done between 12 and 16 weeks of age unless reduced viability necessitates earlier testing. These tests included open field to measure anxiety, activity levels and exploration.

Inverted Screen Testing:

Behavioral screens were performed on a cohort of 4 wild type, 4 heterozygous and 8 homozygous mutant mice. All behavioral tests were done between 12 and 16 weeks of age unless reduced viability necessitates earlier testing. These tests included open field to measure anxiety, activity levels and exploration.

Inverted Screen Test Data:

The Inverted Screen is used to measure motor strength/coordination. Untrained mice were placed individually on top of a square (7.5 cm×7.5 cm) wire screen which was mounted horizontally on a metal rod. The rod was then rotated 180 degrees so that the mice were on the bottom of the screens. The following behavioral responses were recorded over a 1 min testing session: fell off, did not climb, and climbed up.

Results:

| Genotype | Ratio | Fell Down % | Ratio | Climbed up % |
|---|---|---|---|---|
| +/+ (n = 8) | 1/8 | 13 | 3/8 | 38 |
| −/− (n = 8) | 5/8 | 63 | 2/8 | 25 |

A motor strength deficit is apparent when there is a 50% point difference between (−/−) or (+/−) mice and (+/+) mice for the fell down response. 0/8 or 1/8 (−/−) or (+/−) mice not climbing indicates impaired motor coordination. 7/8 or 8/8(−/−) or (+/−) mice climbing up indicates enhanced motor coordination.

The Inverted Screen Test is designed to measure basic sensory & motor observations:

Among the 8 (−/−) mice analyzed, 5 fell off the inverted screen whereas only 1/8 (+/+) mice fell off. These results indicate an impaired motor strength in the mutants. These results are consistent with the observations in bone-related measurements as shown below.

(g) Bone Metabolism & Body Diagnostics

(1) Tissue Mass & Lean Body Mass Measurements—Dexa

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in total tissue mass (TTM).

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI, i.e., whole body, vertebrae, and both femurs).

Body Measurements (Body Length & Weight):

Body Measurements: A measurement of body length and weight was performed at approximately 16 weeks of age.

Results:

1. General Observations: The (−/−) mice appeared smaller than their (+/+) littermates.
2. Weight: The male and female (−/−) mice exhibited decreased mean body weight when compared with their gender-matched (+/+) littermates and the historical means.
3. Length: The male and female (−/−) mice exhibited decreased mean body length when compared with their gender-matched (+/+) littermates and the historical means.

(2) Bone Metabolism: Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Bone MicroCT Analysis:

Procedure: MicroCT was also used to get very sensitive measurements of BMD. One vertebra and 1 femur were taken from a cohort of 4 wild type and 8 homozygous mice. Measurements were taken of lumbar 5 vertebra trabecular bone volume, trabecular thickness, connectivity density and midshaft femur total bone area and cortical thickness. The μCT40 scans provided detailed information on bone mass and architecture. Multiple bones were placed into sample holders and scanned automatically. Instrument software was used to select regions of interest for analysis. Trabecular bone parameters were analyzed in the fifth lumbar vertebrae (LV5) at 16 micrometer resolution and cortical bone parameters were analyzed in the femur midshaft at a resolution of 20 micrometers.

Results:

DEXA: The male and female (−/−) mice exhibited decreased mean total tissue mass when compared with their gender-matched (+/+) littermates. The male and female (−/−) mice exhibited notably decreased bone mineral measurements when compared with their gender-matched (+/+) littermates and the historical means.
2. Micro-CT: The male (−/−) mice exhibited notably decreased mean vertebral trabecular bone volume, number, thickness, and connectivity density and notably decreased mean femoral mid-shaft cross-sectional area and cortical thickness when compared with their gender-matched (+/+) littermates and the historical means.

The (−/−) mice showed signs of growth retardation which could have been due to tissue wasting diseases. In addition, the mutant (−/−) mice analyzed by DEXA and bone micro CT analysis exhibited decreased bone measurements when compared with their (+/+) littermates, suggestive of abnormal bone disorders. The (−/−) mice exhibited a negative bone phenotype with abnormal decreased bone measurements reflective of bone metabolic disorders. The negative bone phenotype indicates that PRO1487polypeptides or agonists thereof would be useful for maintaining bone homeostasis. In addition, PRO1487polypeptides would be useful in bone healing or for the treatment of arthritis or osteoporosis, whereas antagonists (or inhibitors) of PRO1487polypeptides or its encoding gene would lead to abnormal or pathological bone disorders including inflammatory diseases associated with abnormal bone metabolism including arthritis, osteoporosis and osteopenia.

(h) Additional Studies

F1 heterozygous animals were intercrossed to generate F2 wild type, heterozygous, and homozygous progeny. Female fertility was noted as low: 28% (4/14) of the knockout mice were females. Ninety-three percent (93%) (13/14) of the knockout animals developed stubby digits yet none of the heterozygous or wildtype mice showed this phenotype. Stubby digits phenotype was noticed as early as 1-2 weeks old.

Bone related measurements were made using Faxitron X-ray of the paws and spine. Micro-CT analysis showed bone density loss in all the knockout (−/−) mice [bone density of hind and front paws: wildtype=15283; KO=14577]. All wildtype and heterozygous mice have abundant trabecular bone in the marrow space of the proximal femur whereas trabecular bone is decreased in all the knockout mice. Phalangeal bones in the digits of knockout animals are misshapen with cortical thinning and retained cartilage cores [thin cortices, misshappen (possible fusion of phalengeal bones), retained cartilage cores]. The wildtype mice showed normal images of the middle phalangeal bone. Thus, UNQ756 (−/−) mice displayed significant chondrodysplasia. The knockout mice also showed synovial inflammation by local joint tissue microarray analysis and proinflammatory cytokines analysis [hindpaw showed focal necrosis and inflammation; synovial inflammation in the radial carpal joint].

In addition to these bone related studies, the UNQ756 knockout mice showed retinal degeneration with thinning of the outer nuclear layer containing rods and cones. All wildtype and heterozygous animals had normal retinas, whereas all the knockout animals had retinal changes.

In summary, UNQ756 knockout mice clearly showed an arthritis-like phenotype (stubby digits). Developmental defects were noted in the knockout mice including digits misshapen with cortical bone thinning (chondrodysplasia) and retical degeneration. Some of the (−/−) animals showed inflammation lesions. Thus, the UNQ756 knockout mice can serve as a model of arthritis.

70.40. Generation and Analysis of Mice Comprising DNA76399-1700 (UNQ831) Gene Disruptions

In these knockout experiments, the gene encoding PRO1758 polypeptides (designated as DNA76399-1700) (UNQ831) was disrupted. The gene specific information for these studies is as follows: the mutated mouse gene corresponds to nucleotide reference: BC057953 Mus musculus cDNA clone MGC:68074 IMAGE:5340780; the human gene sequence reference: NM_{—}052878 Homo sapiens chromosome 19 open reading frame 36 (C19orf36); the human protein sequence corresponds to reference: Q6UXA2 ACCESSION: Q6UXA2 NID: Homo sapiens (Human). ALLL831.

The mouse gene of interest is represented by a cDNA defined as “Mus musculus cDNA clone MGC:68074 IMAGE:5340780, complete cds,” which is the ortholog of human C19orf36 (chromosome 19 open reading frame 36).

C19orf36 is a putative secreted protein of about 160 amino acids. The protein contains a signal peptide but no other conserved domains.

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 15 | 58 | 15 | 88 |
| Expected | 22 | 44 | 22 | 88 |

Chi-Sq.=4.59 Significance=0.100761384 (hom/n)=0.2 Avg. Litter Size=8

### Mutation Information

Mutation Type: Homologous Recombination (standard)
Description: Coding exons 1 through 4 were targeted (NCBI accession BC057953.1).
1. Wild-type Expression Panel: Expression of the target gene was detected in embryonic stem (ES) cells and in all 13 adult tissue samples tested by RT-PCR, except skeletal muscle and bone.
2. QC Expression: Disruption of the target gene was confirmed by Southern hybridization analysis.

70.40.1. Phenotypic Analysis (for Disrupted Gene: DNA76399-1700 (UNQ831)

(a) Overall Phenotypic Summary:

Mutation of the gene encoding the ortholog of human chromosome 19 open reading frame 36 (C19orf36) resulted in female (−/−) mice exhibiting a decreased skin fibroblast proliferation rate. The female (−/−) mice exhibited an increased mean serum triglyceride level. The mutant knockout (−/−) mice also exhibited an increase in trabecular number and connectivity density but a decrease in midshaft femur total area. Disruption of the target gene was confirmed by Southern hybridization analysis.

(b) Phenotypic Analysis: Cardiology

In the area of cardiovascular biology, targets were identified herein for the treatment of hypertension, atherosclerosis, heart failure, stroke, various coronary artery diseases, dyslipidemias such as high cholesterol (hypercholesterolemia) and elevated serum triglycerides (hypertriglyceridemia), diabetes and/or obesity. The phenotypic tests included the measurement of serum cholesterol and triglycerides.

Blood Lipids

Procedure: A cohort of 4 wild type, 4 heterozygous and 8 homozygous mice were tested in this assay. High cholesterol levels and increased triglyceride blood levels are recognized risk factors in the development of cardiovascular disease and/or diabetes. Measuring blood lipids facilitates the finding of biological switches that regulate blood lipid levels Inhibition of factors which elevate blood lipid levels may be useful for reducing the risk for cardiovascular disease. In these blood chemistry tests, measurements were recorded using the COBAS Integra 400 (mfr: Roche).

Results:

The female (−/−) mice exhibited increased mean serum triglyceride levels when compared with their gender-matched (+/+) littermates and the historical means.

As summarized above, the (−/−) mice exhibited notably increased triglyceride levels when compared with their gender-matched (+/+) littermates and the historical means. Thus, mutant mice deficient in the PRO1758 gene can serve as a model for cardiovascular disease. PRO1758 polypeptides or its encoding gene would be useful in regulating blood lipids such as triglycerides. Thus, PRO1758 polypeptides or agonists thereof would be useful in the treatment of such cardiovascular diseases as hypertension, atherosclerosis, heart failure, stroke, various coronary diseases, hypertriglyceridemia, diabetes and/or obesity.

(c) Adult skin cell proliferation:

Procedure: Skin cells were isolated from 16 week old animals (2 wild type and 4 homozygous mice). These were developed into primary fibroblast cultures and the fibroblast proliferation rates were measured in a strictly controlled protocol. The ability of this assay to detect hyper-proliferative and hypo-proliferative phenotypes has been demonstrated with p53 and Ku80. Proliferation was measured using Brdu incorporation.

Specifically, in these studies the skin fibroblast proliferation assay was used. An increase in the number of cells in a standardized culture was used as a measure of relative proliferative capacity. Primary fibroblasts were established from skin biopsies taken from wild type and mutant mice. Duplicate or triplicate cultures of 0.05 million cells were plated and allowed to grow for six days. At the end of the culture period, the number of cells present in the culture was determined using a electronic particle counter.

Results:

The female (−/−) mice exhibited a decreased mean skin fibroblast proliferation rate when compared with their gender-matched (+/+) littermates.

Thus, homozygous mutant mice demonstrated a hypo-proliferative phenotype. As suggested by these observations, antagonists or inhibitors of PRO1758 polypeptides would mimic this hypo-proliferative phenotype and could function as tumor suppressors and would be useful in decreasing abnormal cell proliferation.

(d) Bone Metabolism & Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Bone microCT Analysis:

Procedure: MicroCT was also used to get very sensitive measurements of BMD. One vertebra and 1 femur were taken from a cohort of 4 wild type and 8 homozygous mice. Measurements were taken of lumbar 5 vertebra trabecular bone volume, trabecular thickness, connectivity density and midshaft femur total bone area and cortical thickness. The μCT40 scans provided detailed information on bone mass and architecture. Multiple bones were placed into sample holders and scanned automatically. Instrument software was used to select regions of interest for analysis. Trabecular bone parameters were analyzed in the fifth lumbar vertebrae (LV5) at 16 micrometer resolution and cortical bone parameters were analyzed in the femur midshaft at a resolution of 20 micrometers.

Results:

Micro-CT: The (−/−) mice exhibited increased trabecular number and connectivity density when compared with their gender-matched (+/+) littermates and the historical means. A decrease in midshaft femur total area was also noted in the (−/−) mice.

70.41. Generation and Analysis of Mice Comprising DNA73775-1707 (UNQ841) Gene Disruptions

In these knockout experiments, the gene encoding PRO1779 polypeptides (designated as DNA73775-1707) (UNQ841) was disrupted. The gene specific information for these studies is as follows: the mutated mouse gene corresponds to nucleotide reference: NM_{—}030175 ACCESSION:NM030175 NID: gi 21313471 ref NM030175.1 Mus musculus RIKEN cDNA 4930507C10 gene (4930507C10Rik); protein reference: Q9D2G9 ACCESSION:Q9D2G9 NID: Mus musculus (Mouse). 4930507C10Rik protein (RIKEN cDNA 4930507C10 gene); the human gene sequence reference: NMO24746 ACCESSION:NMO24746 NID: gi 21362001 ref NMO24746.2 Homo sapiens hypothetical protein FLJ13840 (FLJ13840); the human protein sequence corresponds to reference: Q9H8A0 ACCESSION:Q9H8A0 NID: Homo sapiens (Human). Hypothetical protein FLJ13840.

The mouse gene of interest is RIKEN cDNA 4930507C10 gene, ortholog of human hypothetical protein FLJ13840.

The hypothetical protein is likely to function as an enzyme. The protein contains a domain similar to that found in soluble quinoprotein glucose dehydrogenase from Acinetobacter calcoaceticus (PDB ID: 1 cru). The bacterial enzyme catalyzes the formation of D-glucono-1,5-lactone and ubiquinol from D-glucose and ubiquinone (Oubrie et al, Proc Natl Acad Sci USA 96(21):11787-91 (1999)).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 16 | 47 | 16 | 79 |
| Expected | 19.75 | 39.5 | 19.75 | 79 |

Chi-Sq.=0.39 Significance=0.8228347 (hom/n)=0.23 Avg. Litter Size=9

### Mutation Information

Mutation Type: Homologous Recombination (standard)
Description: Coding exons 2 through 4 were targeted (NCBI accession NM_{—}030 175.1).
1. Wild-type Expression Panel: Expression of the target gene was detected in embryonic stem (ES) cells and in all 13 adult tissue samples tested by RT-PCR, except skeletal muscle and bone.
2. QC Expression: Disruption of the target gene was confirmed by Southern hybridization analysis.

70.41.1. Phenotypic Analysis (for Disrupted Gene: DNA73775-1707 (UNQ841)

(a) Overall Phenotypic Summary:

Mutation of the gene encoding the ortholog of a human hypothetical enzyme resulted in increased pain perception in hot plate testing in the mutant (−/−) mice. The homozygous (−/−) mice also exhibited an increase in total tissue mass, lean body mass, total fat mass (g) and percentages of fat (%) as well as increased bone mineral density measurements. MicroCT measurements were also increased in the mutant (−/−) mice. Gene disruption was confirmed by Southern blot.

(b) Bone Metabolism & Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Bone MicroCT Analysis:

Procedure: MicroCT was also used to get very sensitive measurements of BMD. One vertebra and 1 femur were taken from a cohort of 4 wild type and 8 homozygous mice. Measurements were taken of lumbar 5 vertebra trabecular bone volume, trabecular thickness, connectivity density and midshaft femur total bone area and cortical thickness. The μCT40 scans provided detailed information on bone mass and architecture. Multiple bones were placed into sample holders and scanned automatically. Instrument software was used to select regions of interest for analysis. Trabecular bone parameters were analyzed in the fifth lumbar vertebrae (LV5) at 16 micrometer resolution and cortical bone parameters were analyzed in the femur midshaft at a resolution of 20 micrometers.

Results:

DEXA: The female (−/−) mice exhibited increased mean total tissue mass, lean body mass, and percent total body fat and fat mass as well as increased bone mineral density measurements when compared with their gender-matched (+/+) littermates and the historical means.
Micro-CT: The male (−/−) mice exhibited increased mean femoral mid-shaft cortical thickness when compared with their gender-matched (+/+) littermates and the historical mean.

The (−/−) mice exhibited increased mean total body fat and increased bone mineral density measurements and femoral mid-shaft cortical thickness when compared with their gender-matched (+/+) littermates. These results indicate that the knockout mutant phenotype is associated with such bone abnormalities as osteopetrosis. Osteopetrosis is a condition characterized by abnormal thickening and hardening of bone and abnormal fragility of the bones. As such, PRO1779 polypeptides or agonists thereof would be beneficial for the treatment of osteopetrosis. A phenotype associated with an increased bone mineral content, and total body and femural bone mineral density suggests that agents which mimic these effects (e.g. antagonists of PRO1779 polypeptides) would be useful in bone healing. In addition, female mutant (−/−) mice also exhibited an increased mean percentage of body fat suggestive of an obesity phenotype. These observations suggest that mutant mice deficient in the gene which encodes PRO1779 polypeptides leads to metabolic disorders associated with accumulation of fat but also abnormal bone measurements reflective of general metabolic disorders which can be associated with obesity. Thus, PRO1779 polypeptides or agonists thereof would be useful in the treatment or prevention of such disorders as obesity or other metabolic diseases.

(c) Phenotypic Analysis: CNS/Neurology

In the area of neurology, analysis focused herein on identifying in vivo validated targets for the treatment of neurological and psychiatric disorders including depression, generalized anxiety disorders, attention deficit hyperactivity disorder, obsessive compulsive disorder, schizophrenia, cognitive disorders, hyperalgesia and sensory disorders. Neurological disorders include the category defined as “anxiety disorders” which include but are not limited to: mild to moderate anxiety, anxiety disorder due to a general medical condition, anxiety disorder not otherwise specified, generalized anxiety disorder, panic attack, panic disorder with agoraphobia, panic disorder without agoraphobia, posttraumatic stress disorder, social phobia, specific phobia, substance-induced anxiety disorder, acute alcohol withdrawal, obsessive compulsive disorder, agoraphobia, bipolar disorder I or II, bipolar disorder not otherwise specified, cyclothymic disorder, depressive disorder, major depressive disorder, mood disorder, substance-induced mood disorder. In addition, anxiety disorders may apply to personality disorders including but not limited to the following types: paranoid, antisocial, avoidant behavior, borderline personality disorders, dependent, histronic, narcissistic, obsessive-compulsive, schizoid, and schizotypal.

Procedure:

Behavioral screens were performed on a cohort of 4 wild type, 4 heterozygous and 8 homozygous mice. All behavioral tests were done between 12 and 16 weeks of age unless reduced viability necessitates earlier testing.

Hot Plate Testing

Test Description: The hot plate test for nociception is carried out by placing each mouse on a small enclosed 55° C. hot plate. Latency to a hindlimb response (lick, shake, or jump) is recorded, with a maximum time on the hot plate of 30 sec. Each animal is tested once.

Results:

The (−/−) homozygous (−/−) mice exhibited a decrease latency in the hot plate testing which indicates an increased pain perception compared with their gender-matched wildtype littermates and the historical means.

70.42. Generation and Analysis of Mice Comprising DNA80136-2503 (UNQ847) Gene Disruptions

In these knockout experiments, the gene encoding PRO1785 polypeptides (designated as DNA80136-2503) (UNQ847) was disrupted. The gene specific information for these studies is as follows: the mutated mouse gene corresponds to nucleotide reference: NM_{—}027127 ACCESSION:NM_{—}027127 NID:21312335 Mus musculus Mus musculus RIKEN cDNA 2310016C16 gene (2310016C16Rik); protein reference: Q9D7B7 ACCESSION:Q9D7B7 NID: Mus musculus (Mouse). 2310016C16RIK PROTEIN; the human gene sequence reference: AK074216 ACCESSION: AK074216 NID: 18676756 Homo sapiens Homo sapiens cDNA FLJ23636 fis, clone CAS07176; the human protein sequence corresponds to reference: Q8TED1 ACCESSION:Q8TED1 NID: Homo sapiens (Human). Hypothetical protein FLJ23636 (EPLA847).

The mouse gene of interest is RIKEN cDNA 2310016C16 gene, ortholog of human cDNA FLJ23636. Aliases include “weakly similar to glutathione peroxidase 2.”

FLJ23636 is a member of the glutathione peroxidase family. These selenoenzymes catalyze the glutathione-dependent reduction of hydrogen peroxide and lipid hydroperoxides. FLJ23636 is a type II membrane protein that is predicted to be located in the membrane of the endoplasmic reticulum. The protein consists of a signal anchor and a glutathione peroxidase domain (Pfam accession PF00255). Glutathione peroxidases play an important role in protecting cells from oxidative damage (Miyamoto et al, Biol Chem 384(4):567-74 (2003)).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 16 | 46 | 27 | 89 |
| Expected | 22.25 | 44.5 | 22.25 | 89 |

Chi-Sq.=2.73 Significance=0.25538066 (hom/n)=0.28 Avg. Litter Size=5

### Mutation Information

Mutation Type: Homologous Recombination (standard)
Description: Coding exon 1 was targeted (NCBI accession NM_{—}027127.1).
1. Wild-type Expression Panel: Expression of the target gene was detected in embryonic stem (ES) cells and in all 13 adult tissue samples tested by RT-PCR, except skeletal muscle and bone.
2. QC Expression: Disruption of the target gene was confirmed by Southern hybridization analysis.

70.42.1. Phenotypic Analysis (for Disrupted Gene: DNA80136-2503 (UNQ847)

(a) Overall Phenotypic Summary:

Mutation of the gene encoding the ortholog of a human glutathione peroxidase (FLJ23636) resulted in dermatitis in male (−/−) mice. Microscopic analysis revealed chronic active dermatitis in the male homozygous mutant mice, characterized by epidermal hyperplasia with hyperkeratosis. The homozygotes also exhibited an increase in volumetric bone mineral density and mean total body bone mineral density as well as increased mean femoral midshaft cortical thickness. Decreased mean skin fibroblast proliferation was also observed in the female (−/−) mice. Disruption of the target gene was confirmed by Southern hybridization analysis.

(b) Pathology:

Microscopic Observations: Among the 4 male (−/−) mice analyzed, 3 exhibited chronic multifocal active dermatitis, characterized by epidermal hyperplasia with hyperkeratosis. In the affected male mutants, there were foci consisting of minimal-to-mild chronic active inflammation in the deep dermis, sometimes associated with degeneration and regeneration of the panniculus carnosis muscle. In addition, foreign body granulomas in the deep dermis were associated with ectopic hair shafts. Epithelial ulceration and scabbing were also present in 1/4 (−/−) mice. Mild-to-moderate myeloid hyperplasia was present in the bone marrow of the more severely affected mutants. High levels of antioxidants are associated with the condition of dermatitis. Gene Expression: LacZ activity was not detected in the panel of tissues by immunohistochemical analysis.

(c) Adult Skin Cell Proliferation:

Procedure: Skin cells were isolated from 16 week old animals (2 wild type and 4 homozygous mice). These were developed into primary fibroblast cultures and the fibroblast proliferation rates were measured in a strictly controlled protocol. The ability of this assay to detect hyper-proliferative and hypo-proliferative phenotypes has been demonstrated with p53 and Ku80. Proliferation was measured using Brdu incorporation.

Specifically, in these studies the skin fibroblast proliferation assay was used. An increase in the number of cells in a standardized culture was used as a measure of relative proliferative capacity. Primary fibroblasts were established from skin biopsies taken from wild type and mutant mice. Duplicate or triplicate cultures of 0.05 million cells were plated and allowed to grow for six days. At the end of the culture period, the number of cells present in the culture was determined using a electronic particle counter.

Results:

The female (−/−) mice exhibited a decreased mean skin fibroblast proliferation rate when compared with their gender-matched (+/+) littermates.

Thus, homozygous mutant mice demonstrated a hypo-proliferative phenotype. As suggested by these observations, antagonists or inhibitors of PRO1785 polypeptides would mimic this hypo-proliferative phenotype and could function as tumor suppressors and would be useful in decreasing abnormal cell proliferation.

(d) Bone Metabolism & Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Bone MicroCT Analysis:

Procedure: MicroCT was also used to get very sensitive measurements of BMD. One vertebra and 1 femur were taken from a cohort of 4 wild type and 8 homozygous mice. Measurements were taken of lumbar 5 vertebra trabecular bone volume, trabecular thickness, connectivity density and midshaft femur total bone area and cortical thickness. The μCT40 scans provided detailed information on bone mass and architecture. Multiple bones were placed into sample holders and scanned automatically. Instrument software was used to select regions of interest for analysis. Trabecular bone parameters were analyzed in the fifth lumbar vertebrae (LV5) at 16 micrometer resolution and cortical bone parameters were analyzed in the femur midshaft at a resolution of 20 micrometers.

Results:

DEXA: Both male and female (−/−) mice exhibited increased mean volumetric bone mineral density and mean total body bone mineral density when compared with their gender-matched (+/+) littermates and the historical means.
Micro-CT: The male (−/−) mice exhibited increased mean femoral midshaft cortical thickness and cross-sectional area when compared with their gender-matched (+/+) littermates and the historical means.

In summary, the (−/−) mice exhibited increased bone mineral density measurements and femoral mid-shaft cross-sectional area and thickness when compared with their gender-matched (+/+) littermates. These results indicate that the knockout mutant phenotype is associated with such bone abnormalities as osteopetrosis. Osteopetrosis is a condition characterized by abnormal thickening and hardening of bone and abnormal fragility of the bones. As such, PRO1785 polypeptides or agonists thereof would be beneficial for the treatment of osteopetrosis or other osteo-related diseases. On the other hand, inhibitors or antagonists of PRO1785 polypeptides would be useful in bone healing.

70.43. Generation and Analysis of Mice Comprising DNA77623-2524 (UNQ871) Gene Disruptions

In these knockout experiments, the gene encoding PRO1889 polypeptides (designated as DNA77623-2524) (UNQ871) was disrupted. The gene specific information for these studies is as follows: the mutated mouse gene corresponds to nucleotide reference: XM_{—}128173 PREDICTED: Mus musculus RIKEN cDNA 2300005B03 gene (2300005B03Rik); protein reference: XP_{—}128173 ACCESSION:XP_{—}128173 NID: gi 20902557 ref XP_{—}128173.1 RIKEN cDNA 2300005B03 [Mus musculus]; the human gene sequence reference: NM_{—}177458 Homo sapiens secreted Ly6/uPAR related protein 2 (SLURP2); the human protein sequence corresponds to reference: Q86SR0ACCESSION:Q86SR0NID: Homo sapiens (Human). Secreted Ly6/uPAR related protein 2 (QLGT871).

The mouse gene of interest is RIKEN cDNA 2300005B03 gene, ortholog of human SLURP2 (secreted Ly6/uPAR related protein 2). Aliases include SLURP-2.

SLURP2 is a putative secreted protein that likely functions as a cytokine-like ligand. The protein contains a signal peptide and a Ly-6 antigen/urokinase-type plasminogen receptor-like (Ly6/uPAR) domain. Ly6/uPAR domains are typically found in proteins involved in cellular signaling and immune function (SMART accession SWO134). SLURP2 is expressed in epithelia from several different tissues, including skin and keratinocytes. Although the physiological role of SLURP2 is not clear, it has been implicated in the pathogenesis of psoriasis, possibly playing a role in keratinocyte hyperproliferation, T-cell differentiation, or T-cell activation (Tsuji et al, Genomics 81(1):26-33 (2003); Adermann et al, Protein Sci 8(4):810-9 (1999)).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 13 | 49 | 22 | 84 |
| Expected | 21 | 42 | 21 | 84 |

Chi-Sq.=3.13 Significance=0.209088 (hom/n)=0.29 Avg. Litter Size=8

### Mutation Information

Mutation Type: Homologous Recombination (standard)
Description: Coding exons 2 and 3 were targeted (NCBI accession BY339783).
1. Wild-type Expression Panel: Expression of the target gene was detected in all 13 adult tissue samples tested by RT-PCR, except kidney, skeletal muscle, bone, and adipose.
2. QC Expression: Disruption of the target gene was confirmed by Southern hybridization analysis.

70.43.1. Phenotypic Analysis (for Disrupted Gene: DNA77623-2524 (UNQ871)

(a) Overall Phenotypic Summary:

Mutation of the gene encoding the ortholog of human secreted Ly6/uPAR related protein 2 (SLURP2) resulted in decreased bone mineral density in (−/−) mice. The homozygous mutant mice exhibited decreased bone mineral density when compared with their gender-matched wild-type littermates and the historical means. Hypoactivity was observed in the (−/−) mutant mice but circadian rhythm is still seen. The mutant (−/−) mice also showed decreased heart rates. Disruption of the target gene was confirmed by Southern hybridization analysis.

(b) Phenotypic Analysis: CNS/Neurology

In the area of neurology, analysis focused herein on identifying in vivo validated targets for the treatment of neurological and psychiatric disorders including depression, generalized anxiety disorders, attention deficit hyperactivity disorder, obsessive compulsive disorder, schizophrenia, cognitive disorders, hyperalgesia and sensory disorders. Neurological disorders include the category defined as “anxiety disorders” which include but are not limited to: mild to moderate anxiety, anxiety disorder due to a general medical condition, anxiety disorder not otherwise specified, generalized anxiety disorder, panic attack, panic disorder with agoraphobia, panic disorder without agoraphobia, posttraumatic stress disorder, social phobia, specific phobia, substance-induced anxiety disorder, acute alcohol withdrawal, obsessive compulsive disorder, agoraphobia, bipolar disorder I or II, bipolar disorder not otherwise specified, cyclothymic disorder, depressive disorder, major depressive disorder, mood disorder, substance-induced mood disorder. In addition, anxiety disorders may apply to personality disorders including but not limited to the following types: paranoid, antisocial, avoidant behavior, borderline personality disorders, dependent, histronic, narcissistic, obsessive-compulsive, schizoid, and schizotypal.

Procedure:

Behavioral screens were performed on a cohort of 4 wild type, 4 heterozygous and 8 homozygous mice. All behavioral tests were done between 12 and 16 weeks of age unless reduced viability necessitates earlier testing. These tests included open field to measure anxiety, activity levels and exploration.

Circadian Test Description:

Female mice are individually housed at 4 pm on the first day of testing in 48.2 cm×26.5 cm home cages and administered food and water ad libitum. Animals are exposed to a 12-hour light/dark cycle with lights turning on at 7 am and turning off at 7 pm. The system software records the number of beam interruptions caused by the animal's movements, with beam breaks automatically divided into ambulations. Activity is recorded in 60, one-hour intervals during the three-day test. Data generated are displayed by median activity levels recorded for each hour (circadian rhythm) and median total activity during each light/dark cycle (locomotor activity) over the three-day testing period.

Results:

The female (−/−) mice exhibited decreased ambulatory counts (hypoactivity) during the 1-hour habituation period and both light periods of home-cage activity testing when compared with their gender-matched (+/+) littermates and the historical mean. Circadian rhythm is still seen in the mutant mice. These results are consistent with lethargy or depressive disorders. Antagonists or inhibitors of PRO1889 polypeptides or the PRO1889 encoding gene would be expected to mimic this behavior. Likewise, PRO1889 polypeptides or agonists thereof, would be useful in the treatment of such neurological disorders including depressive disorders or other decreased anxiety-like symptoms such as lethargy, cognitive disorders, hyperalgesia and sensory disorders.

(c) Diagnostics—Heart Rate

Description:

Heart rate is measured via a noninvasive tail-cuff method for four days on the Visitech BP-2000 Blood Pressure Analysis System. Heart rate is measured ten times each day for four days. The four days are then averaged to obtain a mouse's conscious heart rate.

Results:

Both male and female (−/−) mice exhibited decreased mean heart rates (two standard deviations below the historical means) when compared with their gender-matched (+/+) littermates.

(d) Bone Metabolism & Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Results:

DEXA: Both the male and female (−/−) mice exhibited decreased mean volumetric bone mineral density when compared with their gender-matched (+/+) littermates and the historical means.

The (−/−) mice analyzed by DEXA exhibited decreased bone mineral density measurements when compared with their (+/+) littermates, suggestive of abnormal bone disorders. The (−/−) mice exhibited a negative bone phenotype with abnormal decreased bone measurements reflective of bone bone metabolic disorders. The negative bone phenotype indicates that PRO1889 polypeptides or agonists thereof would be useful for maintaining bone homeostasis. In addition, PRO1889 polypeptides would be useful in bone healing or for the treatment of arthritis or osteoporosis, whereas antagonists (or inhibitors) of PRO1889 polypeptides or its encoding gene would lead to abnormal or pathological bone disorders including inflammatory diseases associated with abnormal bone metabolism including arthritis, osteoporosis and osteopenia.

70.44. Generation and Analysis of Mice Comprising DNA336109 (UNQ907) Gene Disruptions

In these knockout experiments, the gene encoding PRO90318 polypeptides (designated as DNA336109) (UNQ907) was disrupted. The gene specific information for these studies is as follows: the mutated mouse gene corresponds to nucleotide reference: NM_{—}138655 Mus musculus transmembrane channel-like gene family 2 (Tmc2); protein reference: Q8R4P4 ACCESSION:Q8R4P4 NID: Mus musculus (Mouse). Transmembrane cochlear-expressed protein 2; the human gene sequence reference: NM_{—}080751 Homo sapiens transmembrane channel-like 2 (TMC2); the human protein sequence corresponds to reference: Q8TDI7 Transmembrane cochlear-expressed protein 2 gi|28642835|gb|AAL86401.2|transmembrane channel-like protein 2 [Homo sapiens].

The mouse gene of interest is Tmc2 (transmembrane channel-like gene family 2), ortholog of human TMC2 (transmembrane channel-like 2). Aliases include transmembrane, cochlear expressed 2; C20orf145; dJ686C3.3; chromosome 20 open reading frame 145; and transmembrane, cochlear expressed, 2.

TMC2 is an integral membrane protein that is likely to function as a modifier of channels or transporters. The protein contains eight transmembrane segments and a TMC signature sequence in the extracellular loop upstream of transmembrane segment six. TMC2 is expressed in cochlea and testis and is a paralog of TMC1 (transmembrane cochlear-expressed gene-1), which is required for cochlear hair cell function. Like TMC1, TMC2 may be involved in mechanoelectrical transduction of sound by cochlear hair cells (Kurima et al, Genomics 82(3):300-8 (2003); Kurima et al, Nat Genet. 30(3):277-84 (2002); Keresztes et al, BMC Genomics 4(1):24 (2003)).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 16 | 38 | 28 | 82 |
| Expected | 20.5 | 41 | 20.5 | 82 |

Chi-Sq.=4.7 Significance=0.09536917 (hom/n)=0.31 Avg. Litter Size=9

### Mutation Information

Mutation Type: Homologous Recombination (standard)
Description: The first coding exon was targeted (Accession: NM_{—}138655).
1. Wild-type Expression Panel: Expression ofthe target gene was detected only in brain among the 13 adult tissue samples tested by RT-PCR.
2. QC Expression: Disruption of the target gene was confirmed by Southern hybridization analysis.

70.44.1. Phenotypic Analysis (for Disrupted Gene: DNA336109 (UNQ907)

(a) Overall Phenotypic Summary:

Mutation of the gene encoding the ortholog of human transmembrane channel-like 2 (TMC2) resulted in the homozygous mutant mice exhibiting decreased hole poking and rearing in open field testing indicative of a decreased exploratory behavior. The (−/−) mice also exhibited an increase IL6 response to LPS. Disruption of the target gene was confirmed by Southern hybridization analysis.

(b) Immunology Phenotypic Analysis

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

T lymphocytes (T cells) are an important component of a mammalian immune response. T cells recognize antigens which are associated with a self-molecule encoded by genes within the major histocompatibility complex (MHC). The antigen may be displayed together with MHC molecules on the surface of antigen presenting cells, virus infected cells, cancer cells, grafts, etc. The T cell system eliminates these altered cells which pose a health threat to the host mammal. T cells include helper T cells and cytotoxic T cells. Helper T cells proliferate extensively following recognition of an antigen-MHC complex on an antigen presenting cell. Helper T cells also secrete a variety of cytokines, i.e., lymphokines, which play a central role in the activation of B cells, cytotoxic T cells and a variety of other cells which participate in the immune response.

In many immune responses, inflammatory cells infiltrate the site of injury or infection. The migrating cells may be neutrophilic, eosinophilic, monocytic or lymphocytic as can be determined by histologic examination of the affected tissues. Current Protocols in Immunology, ed. John E. Coligan, 1994, John Wiley & Sons, Inc.

Many immune related diseases are known and have been extensively studied. Such diseases include immune-mediated inflammatory diseases (such as rheumatoid arthritis, immune mediated renal disease, hepatobiliary diseases, inflammatory bowel disease (IBD), psoriasis, and asthma), non-immune-mediated inflammatory diseases, infectious diseases, immunodeficiency diseases, neoplasia, and graft rejection, etc. In the area of immunology, targets were identified for the treatment of inflammation and inflammatory disorders.

In the area of immunology, targets have been identified herein for the treatment of inflammation and inflammatory disorders. Immune related diseases, in one instance, could be treated by suppressing the immune response. Using neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

The following test was performed:

Acute Phase Response:

Test Description: Bacterial lipopolysaccharide (LPS) is an endotoxin, and as such is a potent inducer of an acute phase response and systemic inflammation. The Level I LPS mice were injected intraperitoneally (i.p.) with a sublethal dose of LPS in 200 μL sterile saline using a 26 gauge needle. The doses were based on the average weight of the mice tested at 1 μg/g body weight 3 hours after injection; a 100u1 blood sample was then taken and analyzed for the presence of TNFa, MCP-1, and IL-6 on the FACSCalibur instrument.

Results:

The (−/−) mice exhibited an increased mean serum IL-6 response to LPS challenge when compared with their (+/+) littermates and the historical mean.

In summary, the LPS endotoxin challenge demonstrated that knockout mice deficient in the gene encoding PRO90318 polypeptides exhibit immunological abnormalities when compared with their wild-type littermates. In particular, the mutant mice exhibited an increased ability to elicit an immunological response (IL-6 production) when challenged with the LPS endotoxin indicating a proinflammatory response. IL-6 contributes to the later stages of B cell activation. In addition, IL-6 plays a critical role in inducing the acute phase response and systemic inflammation. This suggests that inhibitors or antagonists to PRO90318 polypeptides would stimulate the immune system and would find utility in the cases wherein this effect would be beneficial to the individual such as in the case of leukemia, and other types of cancer, and in immunocompromised patients, such as AIDS sufferers. Accordingly, PRO90318 polypeptides or agonists thereof would be useful in inhibiting the immune response and would be useful candidates for suppressing harmful immune responses, e.g. in the case of graft rejection or graft-versus-host diseases.

(c) Phenotypic Analysis: CNS/Neurology

In the area of neurology, analysis focused herein on identifying in vivo validated targets for the treatment of neurological and psychiatric disorders including depression, generalized anxiety disorders, attention deficit hyperactivity disorder, obsessive compulsive disorder, schizophrenia, cognitive disorders, hyperalgesia and sensory disorders. Neurological disorders include the category defined as “anxiety disorders” which include but are not limited to: mild to moderate anxiety, anxiety disorder due to a general medical condition, anxiety disorder not otherwise specified, generalized anxiety disorder, panic attack, panic disorder with agoraphobia, panic disorder without agoraphobia, posttraumatic stress disorder, social phobia, specific phobia, substance-induced anxiety disorder, acute alcohol withdrawal, obsessive compulsive disorder, agoraphobia, bipolar disorder I or II, bipolar disorder not otherwise specified, cyclothymic disorder, depressive disorder, major depressive disorder, mood disorder, substance-induced mood disorder. In addition, anxiety disorders may apply to personality disorders including but not limited to the following types: paranoid, antisocial, avoidant behavior, borderline personality disorders, dependent, histronic, narcissistic, obsessive-compulsive, schizoid, and schizotypal.

Procedure:

Behavioral screens were performed on a cohort of 4 wild type, 4 heterozygous and 8 homozygous mice. All behavioral tests were done between 12 and 16 weeks of age unless reduced viability necessitates earlier testing. These tests included open field to measure anxiety, activity levels and exploration.

Open Field Test:

Several targets of known drugs have exhibited phenotypes in the open field test. These include knockouts of the seratonin transporter, the dopamine transporter (Giros et al., Nature. 1996 Feb. 15; 379(6566):606-12), and the GABA receptor (Homanics et al., Proc Natl Acad Sci USA. 1997 Apr. 15; 94(8):4143-8). An automated open-field assay was customized to address changes related to affective state and exploratory patterns related to learning. First, the field (40×40 cm) was selected to be relatively large for a mouse, thus designed to pick up changes in locomotor activity associated with exploration. In addition, there were 4 holes in the floor to allow for nose-poking, an activity specifically related to exploration. Several factors were also designed to heighten the affective state associated with this test. The open-field test is the first experimental procedure in which the mice are tested, and the measurements that were taken were the subjects' first experience with the chamber. In addition, the open-field was brightly lit. All these factors will heighten the natural anxiety associated with novel and open spaces. The pattern and extent of exploratory activity, and especially the center-to-total distance traveled ratio, may then be able to discern changes related to susceptibility to anxiety or depression. A large arena (40 cm×40 cm, VersaMax animal activity monitoring system from AccuScan Instruments) with infrared beams at three different levels was used to record rearing, hole poke, and locomotor activity. The animal was placed in the center and its activity was measured for 20 minutes. Data from this test was analyzed in five, 4-minute intervals. The total distance traveled (cm), vertical movement number (rearing), number of hole pokes, and the center to total distance ratio were recorded.

The propensity for mice to exhibit normal habituation responses to a novel environment is assessed by determining the overall change in their horizontal locomotor activity across the 5 time intervals. This calculated slope of the change in activity over time is determined using normalized, rather than absolute, total distance traveled. The slope is determined from the regression line through the normalized activity at each of the 5 time intervals. Normal habituation is represented by a negative slope value.

Results:

The (−/−) mice exhibited decreased hole poke and rearing and a decreased exploratory behavior compared to their gender-matched wildtype littermates and historical means.

General & Exploratory Activity: The (−/−) mice exhibited decreased rearing activity and hole poking when compared with their (+/+) littermates, suggesting a decreased exploratory response in the mutants.
Open Field Testing: The (−/−) mice exhibited a decreased exploratory response when compared with their gender-matched (+/+) littermates, which is indicative of a decreased anxiety-like response in the mutants. Thus, knockout mice demonstrated a phenotype consistent with depression, generalized anxiety disorders, cognitive disorders, hyperalgesia and sensory disorders and/or bipolar disorders. Thus, PRO90318 polypeptides and agonists thereof would be useful for the treatment or amelioration of the symptoms associated with depressive disorders.

70.45. Generation and Analysis of Mice Comprising DNA77631-2537 (UNQ1821) Gene Disruptions

In these knockout experiments, the gene encoding PRO3434 polypeptides (designated as DNA77631-2537) (UNQ1821) was disrupted. The gene specific information for these studies is as follows: the mutated mouse gene corresponds to nucleotide reference: NM_{—}026748 Mus musculus RIKEN cDNA 1110015K06 gene (1110015K06Rik); protein reference: Q6P4S8 ACCESSION:Q6P4S8 NID: Mus musculus (Mouse). RIKEN cDNA 1110015K06; the human gene sequence reference: XM_{—}291222 PREDICTED: Homo sapiens DKFZP586J0619 protein (DKFZP586J0619); the human protein sequence corresponds to reference: XP_{—}291222 PREDICTED: DKFZP586J0619 protein [Homo sapiens].

The mouse gene of interest is RIKEN cDNA 1110015K06 gene, ortholog of human DKFZP586J0619 protein.

DKFZP586J0619 protein is a very large hypothetical polypeptide of over 2000 amino acids. Near the C terminus, the mouse protein contains a TAZ zinc finger domain, spanning about 70 amino acids (Pfam accession PF02135). Proteins with this domain include large nuclear molecules CBP and p300, which interact with transcriptional adaptors and suppressors. These transcriptional adaptor molecules are likely to link signal transduction molecules to gene transcription.

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 14 | 53 | 0 | 67 |
| Expected | 16.75 | 33.5 | 16.75 | 767 |

70.45.1. Phenotypic Analysis (for Disrupted Gene: DNA77631-2537 (UNQ1821)

(a) Overall Phenotypic Summary:

Mutation of the gene encoding the ortholog of a human hypothetical protein resulted in lethality of (−/−) mutants. Gene disruption was confirmed by Southern blot.

Discussion Related to Embryonic Developmental Abnormality of Lethality:

Embryonic lethality in knockout mice usually results from various serious developmental problems including but not limited to neurodegenerative diseases, angiogenic disorders, inflammatory diseases, or where the gene/protein has an important role in basic cell signaling processes in many cell types. In addition, embryonic lethals are useful as potential cancer models. Likewise, the corresponding heterozygous (+/−) mutant animals are particularly useful when they exhibit a phenotype and/or a pathology report which reveals highly informative clues as to the function of the knocked-out gene. For instance, EPO knockout animals were embryonic lethals, but the pathology reports on the embryos showed a profound lack of RBCs.

(b) Pathology

Microscopic Observations: Not tested due to embryonic lethality. At 12.5 days, 43 embryos were observed: 24 (+/−) embryos, 13 (+/+) embryos, 2 to-be-determined, and 4 inconclusive.

Gene Expression LacZ activity was not detected in the panel of tissues by immunohistochemical analysis.

70.46. Generation and Analysis of Mice Comprising DNA68862-2546 (UNQ1849) Gene Disruptions

In these knockout experiments, the gene encoding PRO3579 polypeptides (designated as DNA68862-2546) (UNQ1849) was disrupted. The gene specific information for these studies is as follows: the mutated mouse gene corresponds to nucleotide reference: XM_{—}128781 PREDICTED: Mus musculus similar to HSRG1849 (LOC225010); protein reference: XP_{—}128781 similar to HSRG1849 [Mus musculus]; the human gene sequence reference: NM_{—}001002257 Homo sapiens acyl-CoA:lysocardiolipin acyltransferase1 (ALCAT1), transcript variant 2; the human protein sequence corresponds to reference: NP_{—}001002257 acyl-CoA:lysocardiolipin acyltransferase 1 isoform 2; HSRG1849 [Homo sapiens].

The mouse gene of interest is gene model 91, ortholog of human ALCAT1 (acyl-CoA:lysocardiolipin acyltransferase 1). Aliases include UNQ1849, FLJ37965, HSRG1849, and “similar to HSRG1849.”

ALCAT1 is an enzyme located in the endoplasmic reticulum that catalyzes the acyl-CoA-dependent acylation of monolysocardiolipin and dilysocardiolipin. This enzyme is one of several enzymes that remodel cardiolipin, providing the necessary acyl composition for biological activity. Cardiolipin is a membrane polyglycerophospholipid that is required for activity of a number of mitochondrial enzymes involved in energy metabolism (Cao et al, J Biol Chem 279(30):31727-34 (2004)).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 28 | 38 | 26 | 92 |
| Expected | 23 | 46 | 23 | 92 |

Chi-Sq.=0.56 Significance=0.75578374 (hom/n)=0.24 Avg. Litter Size=10

### Mutation Information

Mutation Type: Homologous Recombination (standard)
Description: Coding exon 1 was targeted (NCBI accession XM_{—}128781.5).
1. Wild-type Expression Panel: Expression of the target gene was detected in embryonic stem (ES) cells and in all 13 adult tissue samples tested by RT-PCR, except bone.
2. QC Expression: Disruption of the target gene was confirmed by Southern hybridization analysis.

70.46.1. Phenotypic Analysis (for Disrupted Gene: DNA68862-2546 (UNQ1849)

(a) Overall Phenotypic Summary:

Mutation of the gene encoding the ortholog of human acyl-CoA:lysocardiolipin acyltransferase 1 (ALCAT1) resulted in an increased anxiety-related response in male (−/−) mice. The male homozygous mutant mice exhibited an increased anxiety-like response during stress-induced hyperthermia testing when compared with their gender-matched wild-type littermates and the historical mean. The male (−/−) mice exhibited an increased volumetric bone mineral density and an increased mean femoral mid-shaft cross-sectional area. In addition, the (−/−) mice exhibited an increased total body fat and increased blood triglyceride levels. Disruption of the target gene was confirmed by Southern hybridization analysis.

(b) Phenotypic Analysis: CNS/Neurology

In the area of neurology, analysis focused herein on identifying in vivo validated targets for the treatment of neurological and psychiatric disorders including depression, generalized anxiety disorders, attention deficit hyperactivity disorder, obsessive compulsive disorder, schizophrenia, cognitive disorders, hyperalgesia and sensory disorders. Neurological disorders include the category defined as “anxiety disorders” which include but are not limited to: mild to moderate anxiety, anxiety disorder due to a general medical condition, anxiety disorder not otherwise specified, generalized anxiety disorder, panic attack, panic disorder with agoraphobia, panic disorder without agoraphobia, posttraumatic stress disorder, social phobia, specific phobia, substance-induced anxiety disorder, acute alcohol withdrawal, obsessive compulsive disorder, agoraphobia, bipolar disorder I or II, bipolar disorder not otherwise specified, cyclothymic disorder, depressive disorder, major depressive disorder, mood disorder, substance-induced mood disorder. In addition, anxiety disorders may apply to personality disorders including but not limited to the following types: paranoid, antisocial, avoidant behavior, borderline personality disorders, dependent, histronic, narcissistic, obsessive-compulsive, schizoid, and schizotypal.

Procedure:

Behavioral screens were performed on a cohort of 4 wild type, 4 heterozygous and 8 homozygous mice. All behavioral tests were done between 12 and 16 weeks of age unless reduced viability necessitates earlier testing. These tests included open field to measure anxiety, activity levels and exploration.

Functional Observational Battery (FOB) Test—Stress-induced Hyperthermia

The FOB is a series of situations applied to the animal to determine gross sensory and motor deficits. A subset of tests from the Irwin neurological screen that evaluates gross neurological function is used. In general, short-duration, tactile, olfactory, and visual stimuli are applied to the animal to determine their ability to detect and respond normally. These simple tests take approximately 10 minutes and the mouse is returned to its home cage at the end of testing.

Results:

Anxiety: The male (−/−) mice exhibited increased sensitivity to stress-induced hyperthermia when compared with their gender-matched (+/+) littermates and the historical mean, suggesting an increased anxiety-like response in the mutants. In summary, the functional observation testing revealed a phenotype associated with increased anxiety which could be associated with mild to moderate anxiety, anxiety due to a general medical condition, and/or bipolar disorders; hyperactivity; sensory disorders; obsessive-compulsive disorders, schizophrenia or a paranoid personality. Thus, PRO3579 polypeptides or agonists thereof may play a role in the treatment of such neurological disorders.

(c) Phenotypic Analysis: Cardiology

In the area of cardiovascular biology, targets were identified herein for the treatment of hypertension, atherosclerosis, heart failure, stroke, various coronary artery diseases, dyslipidemias such as high cholesterol (hypercholesterolemia) and elevated serum triglycerides (hypertriglyceridemia), diabetes and/or obesity. The phenotypic tests included the measurement of serum cholesterol and triglycerides.

### Blood Lipids

Procedure: A cohort of 4 wild type, 4 heterozygous and 8 homozygous mice were tested in this assay. High cholesterol levels and increased triglyceride blood levels are recognized risk factors in the development of cardiovascular disease and/or diabetes. Measuring blood lipids facilitates the finding of biological switches that regulate blood lipid levels Inhibition of factors which elevate blood lipid levels may be useful for reducing the risk for cardiovascular disease. In these blood chemistry tests, measurements were recorded using the COBAS Integra 400 (mfr: Roche).

Results:

The (−/−) mice exhibited elevated levels of triglycerides when compared with their gender-matched wildtype littermates and the historical controls.

As summarized above, the (−/−) mice exhibited notably increased triglyceride levels when compared with their gender-matched (+/+) littermates and the historical means. Thus, mutant mice deficient in the PRO3579 gene can serve as a model for cardiovascular disease. PRO3579 polypeptides or its encoding gene would be useful in regulating blood lipids such as triglycerides. Thus, PRO3579 polypeptides or agonists thereof would be useful in the treatment of such cardiovascular diseases as hypertension, atherosclerosis, heart failure, stroke, various coronary diseases, hypertriglyceridemia, diabetes and/or obesity.

(d) Bone Metabolism & Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Results:

DEXA: The male (−/−) mice exhibited increased mean volumetric bone mineral density and total body bone mineral density when compared with their gender-matched (+/+) littermates and the historical means. The (−/−) mice also showed an increased total body fat compared to their gender-matched wildtype littermates.
MicroCT: The male (−/−) mice exhibited an increased mean femoral mid-shaft cross-sectional area when compared with their gender-matched wildtype littermates and the historical means.

The (−/−) mice exhibited increased mean total body fat and increased bone mineral density measurements and femoral mid-shaft cross-sectional area when compared with their gender-matched (+/+) littermates. These results indicate that the knockout mutant phenotype is associated with such bone abnormalities as osteopetrosis. Osteopetrosis is a condition characterized by abnormal thickening and hardening of bone and abnormal fragility of the bones. As such, PRO3579 polypeptides or agonists thereof would be beneficial for the treatment of osteopetrosis. A phenotype associated with an increased bone mineral content, and total body and femural bone mineral density suggests that agents which mimic these effects (e.g. antagonists of PRO3579 polypeptides) would be useful in bone healing. In addition, mutant (−/−) mice also exhibited an increased mean percentage of body fat suggestive of an obesity phenotype. These observations suggest that mutant mice deficient in the gene which encodes PRO3579 polypeptides leads to metabolic disorders associated with accumulation of fat but also abnormal bone measurements reflective of general metabolic disorders which can be associated with obesity. Thus, PRO3579 polypeptides or agonists thereof would be useful in the treatment or prevention of such disorders as obesity or other metabolic diseases.

70.47. Generation and Analysis of Mice Comprising DNA92223-2567 (UNQ1879) Gene Disruptions

In these knockout experiments, the gene encoding PRO4322 polypeptides (designated as DNA92223-2567) (UNQ1879) was disrupted. The gene specific information for these studies is as follows: the mutated mouse gene corresponds to nucleotide reference: NM_{—}145562 ACCESSION:NM_{—}145562 NID: gi 21704107 ref NM_{—}145562.1 Mus musculus similar to DKFZP56400823 protein (LOC231440); protein reference: Q923D3 ACCESSION:Q923D3 NID: Mus musculus (Mouse). Similar to DKFZP56400823 protein (CASTRATION induced pro static apoptosis related protein-1); the human gene sequence reference: AY358777 Homo sapiens clone DNA92223 VYKT1879 (UNQ1879); the human protein sequence corresponds to reference: Q6UWI2 ACCESSION: Q6UWI2 NID: Homo sapiens (Human). VYKT 1879.

The mouse gene of interest is RIKEN cDNA 9130213B05 gene, ortholog of human DKFZP56400823 protein. Aliases include 2210012L08Rik.

DKFZP56400823 protein is a likely type I plasma membrane protein, containing a signal peptide, a transmembrane segment, and a short C terminus. The function of this protein is unknown.

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 16 | 48 | 27 | 91 |
| Expected | 22.75 | 45.5 | 22.75 | 91 |

Chi-Sq.=0.04 Significance=0.9801987 (hom/n)=0.24 Avg. Litter Size=8

### Mutation Information

Mutation Type: Homologous Recombination (standard)
Description: Coding exon 2 was targeted (NCBI accession NM_{—}145562.l).
1. Wild-type Expression Panel: Expression of the target gene was detected in embryonic stem (ES) cells and in all 13 adult tissue samples tested by RT-PCR, except bone.
2. QC Expression: Disruption of the target gene was confirmed by Southern hybridization analysis.
   70.47.1. Phenotypic Analysis (for disrupted gene: DNA92223-2567 (UN01879)

(a) Overall Phenotypic Summary:

Mutation of the gene encoding the ortholog of a human plasma membrane protein (DKFZP56400823) resulted in increased bone-related measurements as well as increase in total body fat. Elevated levels of triglycerides were also observed in both the male and female (−/−) homozygotes. Male knockouts also showed decreased blood glucose levels. Gene disruption was confirmed by Southern blot.

(b) Expression

UNQ1879 is a single transmembrane protein with potential tyrosine phosphorylation sites in the intracellular domain and seems to function as a receptor. Expression is seen in vascular smooth muscle cells and other muscle progenitor cells in murine and human tissues (specifically E11.5 murine embryos; E11.5 midbrain and E11.5 mid trunk).

(c) Phenotypic Analysis: Metabolism-Blood Chemistry

In the area of metabolism, targets may be identified for the treatment of diabetes. Blood chemistry phenotypic analysis includes blood glucose measurements. The COBAS Integra 400 (mfr: Roche) was used for running blood chemistry tests on the mice. In the area of metabolism, targets may be identified for the treatment of diabetes.

Results:

Blood Chemistry: The male (−/−) mice exhibited a decreased mean serum glucose level when compared with their gender-matched (+/+) littermates and the historical mean.
The (−/−) mice exhibited decreased glucose levels when compared with their gender-matched (+/+) littermates and the historical mean. In summary, these knockout mutant mice exhibited a phenotype that is associated with increased insulin sensitivity.

(d) Phenotypic Analysis: Cardiology

In the area of cardiovascular biology, targets were identified herein for the treatment of hypertension, atherosclerosis, heart failure, stroke, various coronary artery diseases, dyslipidemias such as high cholesterol (hypercholesterolemia) and elevated serum triglycerides (hypertriglyceridemia), diabetes and/or obesity. The phenotypic tests included the measurement of serum cholesterol and triglycerides.

Blood Lipids

Procedure: A cohort of 4 wild type, 4 heterozygous and 8 homozygous mice were tested in this assay. High cholesterol levels and increased triglyceride blood levels are recognized risk factors in the development of cardiovascular disease and/or diabetes. Measuring blood lipids facilitates the finding of biological switches that regulate blood lipid levels Inhibition of factors which elevate blood lipid levels may be useful for reducing the risk for cardiovascular disease. In these blood chemistry tests, measurements were recorded using the COBAS Integra 400 (mfr: Roche).

Results:

Both the male and female (−/−) mice exhibited elevated levels of triglycerides (more obvious in the male) when compared with their gender-matched wildtype littermates and the historical controls.

As summarized above, the (−/−) mice exhibited notably increased triglyceride levels when compared with their gender-matched (+/+) littermates and the historical means. Thus, mutant mice deficient in the PRO4322 gene may serve as a model for cardiovascular disease. PRO4322 polypeptides or its encoding gene would be useful in regulating blood lipids such as triglycerides. Thus, PRO4322 polypeptides or agonists thereof would be useful in the treatment of such cardiovascular diseases as hypertension, atherosclerosis, heart failure, stroke, various coronary diseases, hypertriglyceridemia, diabetes and/or obesity.

(e) Bone Metabolism & Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Bone MicroCT Analysis:

Procedure: MicroCT was also used to get very sensitive measurements of BMD. One vertebra and 1 femur were taken from a cohort of 4 wild type and 8 homozygous mice. Measurements were taken of lumbar 5 vertebra trabecular bone volume, trabecular thickness, connectivity density and midshaft femur total bone area and cortical thickness. The μCT40 scans provided detailed information on bone mass and architecture. Multiple bones were placed into sample holders and scanned automatically. Instrument software was used to select regions of interest for analysis. Trabecular bone parameters were analyzed in the fifth lumbar vertebrae (LV5) at 16 micrometer resolution and cortical bone parameters were analyzed in the femur midshaft at a resolution of 20 micrometers.

Results:

DEXA: The male (−/−) exhibited increased mean volumetric bone mineral density and total body bone mineral density when compared with their gender-matched (+/+) littermates and the historical means. The (−/−) mice exhibited increased total body fat compared with their gender-matched wildtype littermates and the historical means.
Micro-CT: The male (−/−) mice exhibited increased mean femoral mid-shaft cross-sectional area when compared with their gender-matched (+/+) littermates and the historical mean.

The (−/−) mice exhibited increased mean total body fat and increased bone mineral density measurements and femoral mid-shaft cross-sectional area when compared with their gender-matched (+/+) littermates. These results indicate that the knockout mutant phenotype is associated with such bone abnormalities as osteopetrosis. Osteopetrosis is a condition characterized by abnormal thickening and hardening of bone and abnormal fragility of the bones. As such, PRO4322 polypeptides or agonists thereof would be beneficial for the treatment of osteopetrosis. A phenotype associated with an increased bone mineral content, and total body and femural bone mineral density suggests that agents which mimic these effects (e.g. antagonists of PRO4322 polypeptides) would be useful in bone healing. In addition, mutant (−/−) mice also exhibited an increased mean percentage of body fat suggestive of an obesity phenotype especially in view of the blood chemistry analysis showing elevated levels of triglycerides in the homozygous mice. These observations suggest that mutant mice deficient in the gene which encodes PRO4322 polypeptides leads to metabolic disorders associated with accumulation of fat but also abnormal bone measurements reflective of general metabolic disorders which can be associated with obesity. Thus, PRO4322 polypeptides or agonists thereof would be useful in the treatment or prevention of such disorders as obesity or other metabolic diseases.

70.48. Generation and Analysis of Mice Comprising DNA92255-2584 (UNQ1897) Gene Disruptions

In these knockout experiments, the gene encoding PRO4343 polypeptides (designated as DNA92255-2584) (UNQ1897) was disrupted. The gene specific information for these studies is as follows: the mutated mouse gene corresponds to nucleotide reference: BC029841 Mus musculus RIKEN cDNA 2010008E23 gene, mRNA (cDNA clone MGC:36813 IMAGE:4209499); protein reference: Q9D8C5 ACCESSION:Q9D8C5 NID: Mus musculus (Mouse). Mus musculus adult male small intestine cDNA, RIKEN full-length enriched library, clone:2010008E23 product:hypothetical Ubiquitin domain containing protein, full insert sequence; the human gene sequence reference: NM_{—}024107 ACCESSION:NM_{—}024107 NID:13129117 Homo sapiens Homo sapiens hypothetical protein MGC3123 (MGC3123); the human protein sequence corresponds to reference: Q71RG4 ACCESSION:Q71RG4 NID: Homo sapiens (Human). FP2653 (ELSD1897) (MGC3123 protein).

The mouse gene of interest is RIKEN cDNA 2010008E23 gene, ortholog of human hypothetical protein MGC3123.

Hypothetical protein MGC3123 is a likely integral plasma membrane protein, consisting of a signal peptide, a ubiquitin homologs domain (SMART accession SM00213), and two C-terminal transmembrane segments. Whether the ubiquitin homologs domain faces the extracellular side or intracellular side of the plasma membrane is not clear. The hypothetical protein may function as a protease inhibitor (GO accession 0004867).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 13 | 37 | 13 | 63 |
| Expected | 15.75 | 31.5 | 15.75 | 63 |

Chi-Sq.=0.39 Significance=0.8228347 (hom/n)=0.24 Avg. Litter Size=8

### Mutation Information

Mutation Type: Homologous Recombination (standard)
Description: Coding exons 1 and 2 were targeted (NCBI accession AK008158.1).
1. Wild-type Expression Panel: Expression of the target gene was detected in embryonic stem (ES) cells and in all 13 adult tissue samples tested by RT-PCR.
2. QC Expression: Disruption of the target gene was confirmed by Southern hybridization analysis.

70.48.1. Phenotypic Analysis (for Disrupted Gene: DNA92255-2584 (UNQ1897)

(a) Overall Phenotypic Summary:

Mutation of the gene encoding the ortholog of a human hypothetical protein (MGC3123) resulted in decreased prepulse inhibition in the (−/−) mice. In addition, the homozygous mice showed an increase in trabecular number and connectivity density. Gene disruption was confirmed by Southern blot.

(b) Phenotypic Analysis: CNS/Neurology

In the area of neurology, analysis focused herein on identifying in vivo validated targets for the treatment of neurological and psychiatric disorders including depression, generalized anxiety disorders, attention deficit hyperactivity disorder, obsessive compulsive disorder, schizophrenia, cognitive disorders, hyperalgesia and sensory disorders. Neurological disorders include the category defined as “anxiety disorders” which include but are not limited to: mild to moderate anxiety, anxiety disorder due to a general medical condition, anxiety disorder not otherwise specified, generalized anxiety disorder, panic attack, panic disorder with agoraphobia, panic disorder without agoraphobia, posttraumatic stress disorder, social phobia, specific phobia, substance-induced anxiety disorder, acute alcohol withdrawal, obsessive compulsive disorder, agoraphobia, bipolar disorder I or II, bipolar disorder not otherwise specified, cyclothymic disorder, depressive disorder, major depressive disorder, mood disorder, substance-induced mood disorder. In addition, anxiety disorders may apply to personality disorders including but not limited to the following types: paranoid, antisocial, avoidant behavior, borderline personality disorders, dependent, histronic, narcissistic, obsessive-compulsive, schizoid, and schizotypal.

Procedure:

Behavioral screens were performed on a cohort of 4 wild type, 4 heterozygous and 8 homozygous mice. All behavioral tests were done between 12 and 16 weeks of age unless reduced viability necessitates earlier testing. These tests included open field to measure anxiety, activity levels and exploration.

Prepulse Inhibition of the Acoustic Startle Reflex

Prepulse inhibition of the acoustic startle reflex occurs when a loud 120 decibel (dB) startle-inducing tone is preceded by a softer (prepulse) tone. The PPI paradigm consists of six different trial types (70 dB background noise, 120 dB alone, 74 dB+120 dB−pp 4, 78 dB+120 dB−pp 8, 82 dB+120 dB−pp 12, and 90 dB+120 dB−pp 20) each repeated in pseudorandom order six times for a total of 36 trials. The max response to the stimulus (V max) is averaged for each trial type. Animals with a 120 dB average value equal to or below 100 are excluded from analysis. The percent that the prepulse inhibits the animal's response to the startle stimulus is calculated and graphed.

Results:

The (−/−) mice exhibited a decreased prepulse inhibition or an enhanced acoustic startle response when compared with their gender-matched wildtype littermates and the historical means.

(c) Bone Metabolism & Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Bone MicroCT Analysis:

Procedure: MicroCT was also used to get very sensitive measurements of BMD. One vertebra and 1 femur were taken from a cohort of 4 wild type and 8 homozygous mice. Measurements were taken of lumbar 5 vertebra trabecular bone volume, trabecular thickness, connectivity density and midshaft femur total bone area and cortical thickness. The μCT40 scans provided detailed information on bone mass and architecture. Multiple bones were placed into sample holders and scanned automatically. Instrument software was used to select regions of interest for analysis. Trabecular bone parameters were analyzed in the fifth lumbar vertebrae (LV5) at 16 micrometer resolution and cortical bone parameters were analyzed in the femur midshaft at a resolution of 20 micrometers.

Results:

MicroCT: The (−/−) mice exhibited an increased trabecular number and connectivity density compared with their wildtype littermates.

The (−/−) mice exhibited increased trabecular bone volume and connectivity density. These results indicate that the knockout mutant phenotype is associated with such bone abnormalities as osteopetrosis. Osteopetrosis is a condition characterized by abnormal thickening and hardening of bone and abnormal fragility of the bones. As such, PRO4343 polypeptides or agonists thereof would be beneficial for the treatment of osteopetrosis. A phenotype associated with an increased bone mineral content, and total body and femoral bone mineral density suggests that agents which mimic these effects (e.g. antagonists of PRO4343 polypeptides) would be useful in bone healing.

70.49. Generation and Analysis of Mice Comprising DNA92288-2588 (UNQ1901) Gene Disruptions

In these knockout experiments, the gene encoding PRO4347 polypeptides (designated as DNA92288-2588) (UNQ1901) was disrupted. The gene specific information for these studies is as follows: the mutated mouse gene corresponds to nucleotide reference: AK003894 Mus musculus 18-day embryo whole body cDNA, RIKEN full-length enriched library, clone: 1110021D20 product:hypothetical Nucleotide-diphospho-sugar transferases structure containing protein, full insert sequence; protein reference: Q9D163 Q9D163 Q9D163 1110021D20 RIK PROTEIN; the human gene sequence reference: NM_{—}031302 Homo sapiens gycosyltransferase (LOC83468); the human protein sequence corresponds to reference: Q9H1C3 ACCESSION:Q9H1C3 NID: Homo sapiens (Human). Glycosyltransferase (Hypothetical protein FLJ31494) (Gycosyltransferase) (ALLR1901).

The mouse gene of interest is RIKEN cDNA 1110021 D₂O gene, ortholog of human “glycosyltransferase.”

The hypothetical protein is a likely type II membrane protein that functions as a glycosyltransferase. The protein consists of a signal anchor and a glycosyltransferase family 8 domain. Members of this family are typically involved in lipopolysaccharide and glycogen synthesis (Pfam accession PF01501). This protein is predicted to be located in the Golgi apparatus or endoplasmic reticulum.

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 28 | 33 | 14 | 75 |
| Expected | 18.75 | 37.5 | 18.75 | 75 |

Chi-Sq.=23.25 Significance=8.939776E-6 (hom/n)=0.15 Avg. Litter Size=8

### Mutation Information

Mutation Type: Homologous Recombination (standard)
Description: Coding exons 3 and 4 were targeted (NCBI accession AK003894.1).
1. Wild-type Expression Panel: Expression of the target gene was detected in brain, spinal cord, thymus, kidney, lung, and adipose among the 13 adult tissue samples tested by RT-PCR.
2. QC Expression: Disruption of the target gene was confirmed by Southern hybridization analysis.

70.49.1. Phenotypic Analysis (for Disrupted Gene: DNA92288-2588 (UNQ1901)

(a) Overall Phenotypic Summary:

Mutation of the gene encoding the ortholog of a human hypothetical glycosyltransferase resulted in decreased serum IgG1 and IgG2a responses to ovalbumin challenge in (−/−) mice. The (−/−) mice exhibited decreased mean body weight and length as well as decreased total tissue mass, total fat mass and total mean percent total body fat (decreased blood triglycerides were also observed in the male (−/−) mice). The male (−/−) mice exhibited decreased femoral bone measurements. Disruption of the target gene was confirmed by Southern hybridization analysis.

(b) Phenotypic Analysis: Cardiology

In the area of cardiovascular biology, targets were identified herein for the treatment of hypertension, atherosclerosis, heart failure, stroke, various coronary artery diseases, dyslipidemias such as high cholesterol (hypercholesterolemia) and elevated serum triglycerides (hypertriglyceridemia), diabetes and/or obesity. The phenotypic tests included the measurement of serum cholesterol and triglycerides.

Blood Lipids

Procedure: A cohort of 4 wild type, 4 heterozygous and 8 homozygous mice were tested in this assay. High cholesterol levels and increased triglyceride blood levels are recognized risk factors in the development of cardiovascular disease and/or diabetes. Measuring blood lipids facilitates the finding of biological switches that regulate blood lipid levels Inhibition of factors which elevate blood lipid levels may be useful for reducing the risk for cardiovascular disease. In these blood chemistry tests, measurements were recorded using the COBAS Integra 400 (mfr: Roche).

Results:

The male (−/−) mice exhibited decreased blood triglyceride levels when compared with their gender-matched wildtype littermates and the historical means.

As summarized above, the (−/−) mice exhibited notably decreased triglyceride levels when compared with their gender-matched (+/+) littermates and the historical means. Thus, mutant mice deficient in the PRO4347 gene can serve as a model for cardiovascular disease. Antagonists or inhibitors of PRO4347 polypeptides or its encoding gene would be useful in regulating blood lipids such as triglycerides. Thus, antagonists of PRO4347 polypeptides would be useful in the treatment of such cardiovascular diseases as hypertension, atherosclerosis, heart failure, stroke, various coronary diseases, hypertriglyceridemia, diabetes and/or obesity.

(c) Bone Metabolism & Body Diagnostics

(1) Tissue Mass & Lean Body Mass Measurements—Dexa

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in total tissue mass (TTM).

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI, i.e., whole body, vertebrae, and both femurs).

Body Measurements (Body Length & Weight):

Body Measurements: A measurement of body length and weight was performed at approximately 16 weeks of age.

Results:

The male (−/−) mice exhibited decreased mean body weight and decreased mean body length when compared with their gender-matched (+/+) llittermates and the historical mean.

(2) Bone Metabolism: Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Bone MicroCT Analysis:

Procedure: MicroCT was also used to get very sensitive measurements of BMD. One vertebra and 1 femur were taken from a cohort of 4 wild type and 8 homozygous mice. Measurements were taken of lumbar 5 vertebra trabecular bone volume, trabecular thickness, connectivity density and midshaft femur total bone area and cortical thickness. The μCT40 scans provided detailed information on bone mass and architecture. Multiple bones were placed into sample holders and scanned automatically. Instrument software was used to select regions of interest for analysis. Trabecular bone parameters were analyzed in the fifth lumbar vertebrae (LV5) at 16 micrometer resolution and cortical bone parameters were analyzed in the femur midshaft at a resolution of 20 micrometers.

Results:

DEXA: The (−/−) mice exhibited decreased mean total tissue mass, total fat mass, and mean percent total body fat when compared with their gender-matched (+/+) littermates.
Micro-CT: The male (−/−) mice exhibited decreased mean femoral mid-shaft cross-sectional area when compared with their gender-matched (+/+) littermates and the historical mean.

Summary:

These results demonstrate that knockout mutant male mice deficient in the gene encoding PRO4347 polypeptides exhibit abnormal growth marked by decreased body weight and body length as well as decreased tissue mass and fat. Deficiencies in bone composition/measurements were also noted in the (−/−) mice, characterized by a decrease in bone femoral mid-shaft cross sectional area and possibly fragility leading to bone fractures. No hypercalcemia, hyperglycemia, or increased alkaline phosphate was detected in blood chemistry tests to suggest renal, parathyroid, or adrenal dysfunction that might be related to the decrease in bone measurements. Thus, it appears that PRO4347 polypeptides or agonists thereof would be useful in preventing growth related disorders as well as promoting bone homeostasis. In addition, PRO4347 polypeptides or its encoding gene would be useful in bone healing or for the treatment of arthritis or osteoporosis; whereas antagonists to PRO4347 polypeptides or its encoding gene would lead to abnormal or pathological bone disorders including inflammatory diseases associated with abnormal bone metabolism such as arthritis, osteoporosis, and osteopenia.

(d) Immunology Phenotypic Analysis

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

T lymphocytes (T cells) are an important component of a mammalian immune response. T cells recognize antigens which are associated with a self-molecule encoded by genes within the major histocompatibility complex (MHC). The antigen may be displayed together with MHC molecules on the surface of antigen presenting cells, virus infected cells, cancer cells, grafts, etc. The T cell system eliminates these altered cells which pose a health threat to the host mammal. T cells include helper T cells and cytotoxic T cells. Helper T cells proliferate extensively following recognition of an antigen-MHC complex on an antigen presenting cell. Helper T cells also secrete a variety of cytokines, i.e., lymphokines, which play a central role in the activation of B cells, cytotoxic T cells and a variety of other cells which participate in the immune response.

In many immune responses, inflammatory cells infiltrate the site of injury or infection. The migrating cells may be neutrophilic, eosinophilic, monocytic or lymphocytic as can be determined by histologic examination of the affected tissues. Current Protocols in Immunology, ed. John E. Coligan, 1994, John Wiley & Sons, Inc.

Many immune related diseases are known and have been extensively studied. Such diseases include immune-mediated inflammatory diseases (such as rheumatoid arthritis, immune mediated renal disease, hepatobiliary diseases, inflammatory bowel disease (IBD), psoriasis, and asthma), non-immune-mediated inflammatory diseases, infectious diseases, immunodeficiency diseases, neoplasia, and graft rejection, etc. In the area of immunology, targets were identified for the treatment of inflammation and inflammatory disorders.

In the area of immunology, targets have been identified herein for the treatment of inflammation and inflammatory disorders. Immune related diseases, in one instance, could be treated by suppressing the immune response. Using neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

The following test was performed:

Ovalbumin Challenge

Procedure: This assay was carried out on 7 wild type and 8 homozygous mice. Chicken ovalbumin (OVA) is a T-cell dependent antigen, which is commonly used as a model protein for studying antigen-specific immune responses in mice. OVA is non-toxic and inert and therefore will not cause harm to the animals even if no immune response is induced. The murine immune response to OVA has been well characterized, to the extent that the immunodominant peptides for eliciting T cell responses have been identified. Anti-OVA antibodies are detectable 8 to 10 days after immunization using enzyme-linked immunosorbent assay (ELIZA), and determination of different isotypes of antibodies gives further information on the complex processes that may lead to a deficient response in genetically engineered mice.

As noted above, this protocol assesses the ability of mice to raise an antigen-specific immune response. Animals were injected IP with 50 mg of chicken ovalbumin emulsified in Complete Feund's Adjuvant and 14 days later the serum titer of anti-ovalbumin antibodies (IgM, IgG1 and IgG2 subclasses) was measured. The amount of OVA-specific antibody in the serum sample is proportional to the Optical Density (OD) value generated by an instrument that scans a 96-well sample plate. Data was collected for a set of serial dilutions of each serum sample.

Results of this Challenge:

The (−/−) mice exhibited decreased mean serum IgG1 and IgG2a responses when compared with their (+/+) littermates and the historical mean.

In summary, the ovalbumin challenge studies indicate that knockout mice deficient in the gene encoding PRO4347 polypeptides exhibit immunological abnormalities when compared with their wild-type littermates. In particular, the mutant mice exhibited a decreased ability to elicit an immunological response when challenged with the T-cell dependent OVA antigen. Thus, PRO4347 polypeptides or agonists thereof, would be useful for stimulating the immune system (such as T cell proliferation) and would find utility in the cases wherein this effect would be beneficial to the individual such as in the case of leukemia, and other types of cancer, and in immunocompromised patients, such as AIDS sufferers. Accordingly, inhibitors (antagonists) of PRO4347 polypeptides would be useful for inhibiting the immune response and thus would be useful candidates for suppressing harmful immune responses, e.g. in the case of graft rejection or graft-versus-host diseases.

70.50. Generation and Analysis of Mice Comprising DNA83509-2612 (UNQ1928) Gene Disruptions

In these knockout experiments, the gene encoding PRO4403 polypeptides (designated as DNA83509-2612) (UNQ1928) was disrupted. The gene specific information for these studies is as follows: the mutated mouse gene corresponds to nucleotide reference: NM_{—}183297 Mus musculus neurexophilin 4 (Nxph4); protein reference: Q8BTD1 ACCESSION:Q8BTD1 NID: Mus musculus (Mouse). Neurexophilin 4. MOUSESPTRNRDB; the human gene sequence reference: NM_{—}007224 Homo sapiens neurexophilin 4 (NXPH4); the human protein sequence corresponds to reference: Q7Z6L3 ACCESSION:Q7Z6L3 NID: Homo sapiens (Human). Neurexophilin 4 (NXPH4).

The mouse gene of interest is Nxph4 (neurexophilin 4), ortholog of human NXPH4. Aliases include 1110036M10Rik and NPH4.

NXPH4 is likely to be a secreted neuropeptide-like glycoprotein that functions as a receptor ligand. NXPH4 consists of a signal peptide, a variable N-terminal domain, a highly conserved N-glycosylated central domain, a short linker region, and a conserved cysteine-rich C-terminal domain. In neural cell lines, neurexophilins appear to be processed like other neuropeptide hormones by endoproteolytic cleavage. Neurexophilin family members 1 and 3 bind with alpha-neurexins, neural membrane proteins that likely function as cell-surface receptors. In contrast, NXPH4 does not bind with alpha-neurexins, and the endogenous receptor for NXPH4 remains unknown (Missler and Sudhof, J Neurosci 18(10):3630-8 (1998); Missler et al, J Biol Chem 273(52):34716-23 (1998)).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 23 | 46 | 23 | 92 |
| Expected | 23 | 46 | 23 | 92 |

Chi-Sq.=1.32 Significance=0.5168513 (hom/n)=0.27 Avg. Litter Size=10

### Mutation Information

Mutation Type: Homologous Recombination (standard)
Description: Coding exon 2 was targeted (NCBI accession NM_{—}183297.1).
1. Wild-type Expression Panel: Expression of the target gene was detected in embryonic stem (ES) cells and, among 13 adult tissue samples tested by RT-PCR, in brain, spinal cord, eye, kidney, and liver.
2. QC Expression: Disruption of the target gene was confirmed by Southern hybridization analysis.

70.50.1. Phenotypic Analysis (for Disrupted Gene: DNA83509-2612 (UNQ1928)

(a) Overall Phenotypic Summary:

Mutation of the gene encoding the ortholog of human neurexophilin 4 (NXPH4) resulted in a decreased serum triglyceride level in female (−/−) mice. The (−/−) mice also exhibited nitrituria. In addition, the (−/−) mice exhibited decreased glucose tolerance. Both the male and female (−/−) mice showed decreased mean body length. The male (−/−) mice exhibited notably decreased mean bone mineral density in vertebrae. Disruption of the target gene was confirmed by Southern hybridization analysis.

(b) Phenotypic Analysis: Cardiology

In the area of cardiovascular biology, targets were identified herein for the treatment of hypertension, atherosclerosis, heart failure, stroke, various coronary artery diseases, dyslipidemias such as high cholesterol (hypercholesterolemia) and elevated serum triglycerides (hypertriglyceridemia), diabetes and/or obesity. The phenotypic tests included the measurement of serum cholesterol and triglycerides.

Blood Lipids

Procedure: A cohort of 4 wild type, 4 heterozygous and 8 homozygous mice were tested in this assay. High cholesterol levels and increased triglyceride blood levels are recognized risk factors in the development of cardiovascular disease and/or diabetes. Measuring blood lipids facilitates the finding of biological switches that regulate blood lipid levels Inhibition of factors which elevate blood lipid levels may be useful for reducing the risk for cardiovascular disease. In these blood chemistry tests, cholesterol measurements were recorded using the COBAS Integra 400 (mfr: Roche).

Results:

The female homozygous (−/−) mice exhibited a decreased mean serum triglyceride level when compared with their gender-matched (+/+) littermates and the historical means.

Thus, mutant mice deficient in the PRO4403 encoding gene can serve as a model for treatment of cardiovascular disease especially those diseases which are associated with dyslipidemia Inhibitors (antagonists) of PRO4403 polypeptides or its encoding gene would be useful in regulating blood lipids and in particular for maintaining normal levels of triglycerides. Thus, antagonists of PRO4403 polypeptides would be useful in the treatment of such cardiovascular diseases as: hypertension, atherosclerosis, heart failure, stroke, various coronary artery diseases, and/or obesity or diabetes.

(c) Phenotypic Analysis: Metabolism-Blood Chemistry/Glucose Tolerance

In the area of metabolism, targets may be identified for the treatment of diabetes. Blood chemistry phenotypic analysis includes blood glucose measurements. The COBAS Integra 400 (mfr: Roche) was used for running blood chemistry tests on the mice. In the area of metabolism, targets may be identified for the treatment of diabetes. Blood chemistry phenotypic analysis includes glucose tolerance tests to measure insulin sensitivity and changes in glucose metabolism. Abnormal glucose tolerance test results may indicate but may not be limited to the following disorders or conditions: Diabetes Type 1 and Type 2, Syndrome X, various cardiovascular diseases and/or obesity.

Procedure: A cohort of 2 wild type and 4 homozygous mice were used in this assay. The glucose tolerance test is the standard for defining impaired glucose homeostasis in mammals. Glucose tolerance tests were performed using a Lifescan glucometer. Animals were injected IP at 2 g/kg with D-glucose delivered as a 20% solution and blood glucose levels were measured at 0, 30, 60 and 90 minutes after injection.

Results:

Glucose Tolerance Test: The mutant (−/−) mice tested exhibited a decreased or impaired glucose tolerance when compared with their gender-matched (+/+) littermates.

These studies indicated that (−/−) mice exhibit a decreased or impaired glucose tolerance in the presence of normal fasting glucose at all 3 intervals tested when compared with their gender-matched (+/+) littermates and the historical means. Thus, knockout mutant mice exhibited the phenotypic pattern of an impaired glucose homeostasis, and therefore PRO4403 polypeptides (or agonists thereof) or its encoding gene would be useful in the treatment of conditions associated with an impaired glucose homeostasis and/or various cardiovascular diseases, including diabetes.

Urinalysis

Description:

The routine urinalysis is a screening test done to provide a general evaluation of the renal/urinary system. The characteristics for which urine is routinely examined includes tests for protein, glucose, ketones, blood, bilirubin, urobilinogen, nitrate and leukocyte esterase, as well as pH and specific gravity.

Results:

Of the 8 (−/−) mice analyzed, 7 exhibited nitrituria.

(d) Bone Metabolism & Body Diagnostics

(1) Tissue Mass & Lean Body Mass Measurements—Dexa

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in total tissue mass (TTM).

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI, i.e., whole body, vertebrae, and both femurs).

Body Measurements (Body Length & Weight):

Body Measurements: A measurement of body length and weight was performed at approximately 16 weeks of age.

Results:

The male and female (−/−) mice exhibited decreased mean body length when compared with their gender-matched (+/+) littermates.

(2) Bone Metabolism: Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Results:

DEXA: The male (−/−) mice exhibited notably decreased mean bone mineral density in vertebrae when compared with their gender-matched (+/+) littermates and the historical mean.

The (−/−) mice analyzed by DEXA exhibited decreased bone measurements when compared with their (+/+) littermates, suggestive of abnormal bone disorders. The (−/−) mice also showed decreased body length measurements which could be associated with growth related disorders. The (−/−) mice exhibited a negative bone phenotype with abnormal and decreased bone measurements reflective of bone metabolic disorders. The negative bone phenotype indicates that PRO4403 polypeptides or agonists thereof would be useful for maintaining bone homeostasis. In addition, PRO4403 polypeptides would be useful in bone healing or for the treatment of arthritis or osteoporosis, whereas antagonists (or inhibitors) of PRO4403 polypeptides or its encoding gene would lead to abnormal or pathological bone disorders including inflammatory diseases associated with abnormal bone metabolism including arthritis, osteoporosis and osteopenia.

70.51. Generation and Analysis of Mice Comprising DNA100902-2646 (UNQ2419) Gene Disruptions

In these knockout experiments, the gene encoding PRO4976 polypeptides (designated as DNA100902-2646) (UNQ2419) was disrupted. The gene specific information for these studies is as follows: the mutated mouse gene corresponds to nucleotide reference: NM_{—}172282 Mus musculus RIKEN cDNA B230339H12 gene (B230339H12Rik); protein reference: Q8BH01 ACCESSION:Q8BH01 NID: Mus musculus (Mouse). Mus musculus adult male colon cDNA, RIKEN full-length enriched library, clone:9030405H12 product:hypothetical Na/H exchanger containing protein, full insert sequence (RIKEN cDNA B230339H12) (Mus musculus 2 days neonate thymus thymic cells cDNA, RIKEN full-length enriched library, clone:E430029F23 product:hypothetical Na/H exchanger containing protein, full insert sequence); the human gene sequence reference: NM_{—}017905 Homo sapiens chromosome 13 open reading frame 11 (C13orf11); the human protein sequence corresponds to reference: Q6UWJ1 ACCESSION:Q6UWJ1 NID: Homo sapiens (Human). C13orf11.

The mouse gene of interest is RIKEN cDNA B230339H12 gene, ortholog of human C13orf11 (chromosome 13 open reading frame 11). Aliases include FLJ20623 and B230339H12Rik.

C13orf11 is a hypothetical integral plasma membrane protein that is likely to function as a proton/sodium antiporter. C13orf11 consists of a signal peptide, a coiled-coil region, and 10 transmembrane segments contained within a sodium/hydrogen exchanger domain. Proteins with this domain are likely to play a role in regulating intracellular pH, extruding protons generated during metabolism (Pfam accession PF00999).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 18 | 37 | 19 | 74 |
| Expected | 18.5 | 37 | 18.5 | 74 |

Chi-Sq.=0.19 Significance=0.9093729 (hom/n)=0.25 Avg. Litter Size=8

### Mutation Information

Mutation Type: Homologous Recombination (standard)
Description: Coding exons 1 through 3 were targeted (NCBI accession NM_{—}172282.1).
1. Wild-type Expression Panel: Expression of the target gene was detected in embryonic stem (ES) cells and in all 13 adult tissue samples tested by RT-PCR.
2. QC Expression: Disruption of the target gene was confirmed by Southern hybridization analysis.

70.51.1. Phenotypic Analysis (for Disrupted Gene: DNA100902-2646 (UNQ2419)

(a) Overall Phenotypic Summary:

Mutation of the gene encoding the ortholog of human chromosome 13 open reading frame 11 (C13orf11) resulted in hypoactivity and increased immobility during neurological testing in the (−/−) mice. The male (−/−) mice exhibited notably decreased lean body mass and decreased mean femoral mid-shaft cross-sectional area. Gene disruption was confirmed by Southern blot.

(b) Bone Metabolism & Body Diagnostics

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Bone MicroCT Analysis:

Procedure: MicroCT was also used to get very sensitive measurements of BMD. One vertebra and 1 femur were taken from a cohort of 4 wild type and 8 homozygous mice. Measurements were taken of lumbar 5 vertebra trabecular bone volume, trabecular thickness, connectivity density and midshaft femur total bone area and cortical thickness. The μCT40 scans provided detailed information on bone mass and architecture. Multiple bones were placed into sample holders and scanned automatically. Instrument software was used to select regions of interest for analysis. Trabecular bone parameters were analyzed in the fifth lumbar vertebrae (LV5) at 16 micrometer resolution and cortical bone parameters were analyzed in the femur midshaft at a resolution of 20 micrometers.

Results:

DEXA: The male (−/−) mice exhibited notably decreased mean lean body mass when compared with their gender-matched (+/+) littermates and the historical means.
Micro-CT: The male (−/−) mice exhibited decreased mean femoral mid-shaft cross-sectional area when compared with their gender-matched (+/+) littermates and the historical means.

The (−/−) mice analyzed by DEXA and bone micro CT analysis exhibited decreased body mass and bone measurements when compared with their (+/+) littermates, suggestive of abnormal bone disorders. The negative bone phenotype indicates that PRO4976 polypeptides or agonists thereof would be useful for maintaining bone homeostasis. In addition, PRO4976 polypeptides would be useful in bone healing or for the treatment of arthritis or osteoporosis, whereas antagonists (or inhibitors) of PRO4976 polypeptides or its encoding gene would lead to abnormal or pathological bone disorders including inflammatory diseases associated with abnormal bone metabolism including arthritis, osteoporosis and osteopenia.

(c) Phenotypic Analysis: CNS/Neurology

In the area of neurology, analysis focused herein on identifying in vivo validated targets for the treatment of neurological and psychiatric disorders including depression, generalized anxiety disorders, attention deficit hyperactivity disorder, obsessive compulsive disorder, schizophrenia, cognitive disorders, hyperalgesia and sensory disorders. Neurological disorders include the category defined as “anxiety disorders” which include but are not limited to: mild to moderate anxiety, anxiety disorder due to a general medical condition, anxiety disorder not otherwise specified, generalized anxiety disorder, panic attack, panic disorder with agoraphobia, panic disorder without agoraphobia, posttraumatic stress disorder, social phobia, specific phobia, substance-induced anxiety disorder, acute alcohol withdrawal, obsessive compulsive disorder, agoraphobia, bipolar disorder I or II, bipolar disorder not otherwise specified, cyclothymic disorder, depressive disorder, major depressive disorder, mood disorder, substance-induced mood disorder. In addition, anxiety disorders may apply to personality disorders including but not limited to the following types: paranoid, antisocial, avoidant behavior, borderline personality disorders, dependent, histronic, narcissistic, obsessive-compulsive, schizoid, and schizotypal.

Procedure:

Behavioral screens were performed on a cohort of 4 wild type, 4 heterozygous and 8 homozygous mice. All behavioral tests were done between 12 and 16 weeks of age unless reduced viability necessitates earlier testing. These tests included open field to measure anxiety, activity levels and exploration.

Open Field Test:

Several targets of known drugs have exhibited phenotypes in the open field test. These include knockouts of the seratonin transporter, the dopamine transporter (Giros et al., Nature. 1996 Feb. 15; 379(6566):606-12), and the GABA receptor (Homanics et al., Proc Natl Acad Sci USA. 1997 Apr. 15; 94(8):4143-8). An automated open-field assay was customized to address changes related to affective state and exploratory patterns related to learning. First, the field (40×40 cm) was selected to be relatively large for a mouse, thus designed to pick up changes in locomotor activity associated with exploration. In addition, there were 4 holes in the floor to allow for nose-poking, an activity specifically related to exploration. Several factors were also designed to heighten the affective state associated with this test. The open-field test is the first experimental procedure in which the mice are tested, and the measurements that were taken were the subjects' first experience with the chamber. In addition, the open-field was brightly lit. All these factors will heighten the natural anxiety associated with novel and open spaces. The pattern and extent of exploratory activity, and especially the center-to-total distance traveled ratio, may then be able to discern changes related to susceptibility to anxiety or depression. A large arena (40 cm×40 cm, VersaMax animal activity monitoring system from AccuScan Instruments) with infrared beams at three different levels was used to record rearing, hole poke, and locomotor activity. The animal was placed in the center and its activity was measured for 20 minutes. Data from this test was analyzed in five, 4-minute intervals. The total distance traveled (cm), vertical movement number (rearing), number of hole pokes, and the center to total distance ratio were recorded.

The propensity for mice to exhibit normal habituation responses to a novel environment is assessed by determining the overall change in their horizontal locomotor activity across the 5 time intervals. This calculated slope of the change in activity over time is determined using normalized, rather than absolute, total distance traveled. The slope is determined from the regression line through the normalized activity at each of the 5 time intervals. Normal habituation is represented by a negative slope value.

Results:

The (−/−) mice exhibited decreased hole poking and rearing and general hypoactivity (less distance traveled) during open field testing when compared with their gender-matched wildtype littermates and the historical means.

General & Exploratory Activity: The (−/−) mice exhibited decreased rearing activity and hole poking when compared with their (+/+) littermates, suggesting a decreased exploratory response in the mutants.
Open Field Testing: The (−/−) mice exhibited hypoactivity and a decreased exploratory response when compared with their gender-matched (+/+) littermates. These observations are indicative of a decreased anxiety-like response in the mutants. Thus, knockout mice demonstrated a phenotype consistent with depression, generalized anxiety disorders, cognitive disorders, hyperalgesia and sensory disorders and/or bipolar disorders. Thus, PRO4976 polypeptides and agonists thereof would be useful for the treatment or amelioration of the symptoms associated with depressive disorders.

Tail Suspension Testing:

The tail suspension test is a procedure that has been developed as a model for depressive-like behavior in rodents. In this particular setup, a mouse is suspended by its tail for 6 minutes, and in response the mouse will struggle to escape from this position. After a certain period of time the struggling of the mouse decreases and this is interpreted as a type of learned helplessness paradigm. Animals with invalid data (i.e. climbed their tail during the testing period) are excluded from analysis.

Results:

The (−/−) mice showed an increased response time during the tail suspension testing. These results are indicative of a decrease in learned helplessness or increased immobility. Thus, the mutant mice show an increased depressive-like behavior.

70.52. Generation and Analysis of Mice Comprising DNA33470-1175 (UNQ227) Gene Disruptions

In these knockout experiments, the gene encoding PRO260 polypeptides (designated as DNA33470-1175) (UNQ227) was disrupted. The gene specific information for these studies is as follows: the mutated mouse gene corresponds to nucleotide reference: NM_{—}025799 Mus musculus RIKEN cDNA 0610025011 gene (0610025011Rik); protein reference: Q99KR8ACCESSION:Q99KR8NID: Mus musculus (Mouse). RIKEN cDNA 0610025011 GENE; the human gene sequence reference:NM032020 Homo sapiens fucosidase, alpha-L-2, plasma (FUCA2); the human protein sequence corresponds to reference:. Q9BTY2 ACCESSION:Q9BTY2 NID: Homo sapiens (Human). Similar to fucosidase, alpha-L-1, tissue (MGC1314 protein).

The mouse gene of interest is RIKEN cDNA 0610025011 gene, ortholog of human FUCA2 (fucosidase, alpha-L-2, plasma). Aliases include MGC1314, dJ20N2.5, and 5530401P20Rik. FUCA2 is a plasma enzyme that catalyzes the hydrolysis of terminal alpha-L-fucose linkages in glycoproteins and glycosphingolipids. The enzyme likely plays a role in N-glycan degradation (Johnson and Alhadeff, Comp Biochem Physiol B 99(3):479-88 (1991); Eiberg et al, Clin Genet. 26(1):23-9 (1984)).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 24 | 35 | 17 | 76 |
| Expected | 19 | 38 | 19 | 76 |

Chi-Sq.=0.89 Significance=0.64082426 (hom/n)=0.25 Avg. Litter Size=11

### Mutation Information

Mutation Type: Hornologous Recornbination (standard)
Description: Coding exons 1 and 2 were targeted (NCBI accession NM_{—}025799.2).
1. Wild-type Expression Panel: Expression of tbe target gene was detected in ernbryonic stern (ES) cells and in all 13 adult tissue sarnples tested by RT-PCR, except bone and adipose.
2. QC Expression: Disruption of the target gene was confirmed by Soutbern bybridization analysis.

70.52.1. Phenotypic Analysis (for Disrupted Gene: DNA33470-1175 (UNQ227)

(a) Overall Phenotypic Summary:

Mutation of the gene encoding the ortholog of human fucosidase, alpha-L-2, plasma (FUCA2) resulted in increased total tissue mass, fat (%) and fat (g) in mutant homozygous mice. Gene disruption was confirmed by Southern blot.

(b) Bone Metabolism & Body Diagnostics

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Results:

DEXA: The (−/−) mice exhibited an increased total tissue mass, total body fat mass (g) and toal mean percent body fat (%) compared with their gender-matched wildtype littermates and the historical means.

These studies suggest that mutant (−/−) non-human transgenic animals exhibit a negative phenotype that would be associated with obesity. Thus, PRO260 polypeptides or agonists thereof are essential for normal growth and metabolic processes and especially would be important in the prevention and/or treatment of obesity.

70.53. Generation and Analysis of Mice Comprising DNA92217-2697 (UNQ2521) Gene Disruptions

In these knockout experiments, the gene encoding PRO6014 polypeptides (designated as DNA92217-2697) (UNQ2521) was disrupted. The gene specific information for these studies is as follows: the mutated mouse gene corresponds to nucleotide reference: NM_{—}138750 ACCESSION:NM_{—}138750 NID:20270282 Mus musculus Mus musculus prominin-related protein (Prom-rp); protein reference: Q8R4Y7 ACCE S SION: Q8R4Y7 NID: Mus musculus (Mouse). Prominin-2; the human gene sequence reference: NM_{—}144707 Homo sapiens prominin 2 (PROM2); the human protein sequence corresponds to reference: Q8TAE2 ACCESSION:Q8TAE2 NID: Homo sapiens (Human). Prominin-2 variant A (PROM2) (Prominin-2 variant B).

The mouse gene of interest is Prom2 (prominin 2), ortholog of human PROM2. Aliases include PROM-2, Prom-rp, MGC31164, and prominin-related protein.

PROM2 is an integral membrane protein found in protrusions of the plasma membrane in epithelial and non-epithelial cells. The protein consists of an extracellular N-terminal segment, five transmembrane domains, and a cytoplasmic C-terminal segment. The protein colocalizes with family member prominin-1, which is associated with retinal degeneration in humans. PROM2 is expressed in kidney, the digestive tract, and other epithelia. Unlike prominin-1, PROM2 is not expressed in the eye (Fargeas et al, J Biol Chem 278(10):8586-96 (2003)).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 23 | 42 | 17 | 82 |
| Expected | 20.5 | 41 | 20.5 | 82 |

Chi-Sq.=0.47 Significance=0.79057086 (hom/n)=0.25 Avg. Litter Size=8

### Mutation Information

Mutation Type: Homologous Recombination (standard)
Description: Coding exons 1 through 6 were targeted (NCBI accession NM_{—}138750.1).
1. Wild-type Expression Panel: Expression of the target gene was detected in embryonic stem (ES) cells and in all 13 adult tissue samples tested by RT-PCR, except skeletal muscle, bone, and heart.
2. QC Expression: Disruption of the target gene was confirmed by Southern hybridization analysis.

70.53.1. Phenotypic Analysis (for Disrupted Gene: DNA92217-2697 (UNQ2521)

(a) Overall Phenotypic Summary:

Mutation of the gene encoding the ortholog of human prominin 2 (PROM2) resulted in anemia in (−/−) mice. The homozygous mutant mice exhibited signs of anemia, including a decreased mean red blood cell count and decreased mean hemoglobin and hematocrit levels, when compared with their wild-type littermates and the historical means. The (−/) mice also showed decreased serum glucose levels, however glucose tolerance testing was normal. The mutant (−/−) mice showed decreased mean body weight and mean body length. Disruption of the target gene was confirmed by Southern hybridization analysis.

(b) Immunology Phenotypic Analysis

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

T lymphocytes (T cells) are an important component of a mammalian immune response. T cells recognize antigens which are associated with a self-molecule encoded by genes within the major histocompatibility complex (MHC). The antigen may be displayed together with MHC molecules on the surface of antigen presenting cells, virus infected cells, cancer cells, grafts, etc. The T cell system eliminates these altered cells which pose a health threat to the host mammal. T cells include helper T cells and cytotoxic T cells. Helper T cells proliferate extensively following recognition of an antigen-MHC complex on an antigen presenting cell. Helper T cells also secrete a variety of cytokines, i.e., lymphokines, which play a central role in the activation of B cells, cytotoxic T cells and a variety of other cells which participate in the immune response.

In many immune responses, inflammatory cells infiltrate the site of injury or infection. The migrating cells may be neutrophilic, eosinophilic, monocytic or lymphocytic as can be determined by histologic examination of the affected tissues. Current Protocols in Immunology, ed. John E. Coligan, 1994, John Wiley & Sons, Inc.

Many immune related diseases are known and have been extensively studied. Such diseases include immune-mediated inflammatory diseases (such as rheumatoid arthritis, immune mediated renal disease, hepatobiliary diseases, inflammatory bowel disease (IBD), psoriasis, and asthma), non-immune-mediated inflammatory diseases, infectious diseases, immunodeficiency diseases, neoplasia, and graft rejection, etc. In the area of immunology, targets were identified for the treatment of inflammation and inflammatory disorders.

In the area of immunology, targets have been identified herein for the treatment of inflammation and inflammatory disorders. Immune related diseases, in one instance, could be treated by suppressing the immune response. Using neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

The following tests were performed:

Hematology Analysis:

Test Description: Blood tests are carried out by Abbott's Cell-Dyn 3500R, an automated hematology analyzer. Some of its features include a five-part WBC differential. ‘Patient’ reports can cover over 22 parameters in all.

Results:

The (−/−) mice exhibited a decreased mean total red blood cell count, hemoglobin level, and hematocrit levels when compared with their (+/+) littermates and the historical means.

The mutant (−/−) mice exhibited a phenotype associated with anemia. Thus, PRO6014 polypeptides, agonists thereof or the encoding gene for PRO6014 polypeptides must be essential for normal red blood cell production and as such would be useful in the treatment of blood disorders associated with anemia or a low hematocrit.

(c) Bone Metabolism & Body Diagnostics

Tissue Mass & Lean Body Mass Measurements—Dexa

Dexa Analysis—Test Description:

Procedure: A cohort of 4 wild type, 4 heterozygous and 8 homozygous mice were tested in this assay.

Dual Energy X-ray Absorptiometry (DEXA) has been used successfully to identify changes in total tissue mass (TTM).

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI, i.e., whole body, vertebrae, and both femurs).

Body Measurements (Body Length & Weight):

Body Measurements: A measurement of body length and weight was performed at approximately 16 weeks of age.

Results:

The (−/−) mice exhibited decreased mean body weight and mean body length when compared with their gender-matched (+/+) littermates and the historical mean. Thus, body measurements indicated growth related problems.

(d) Phenotypic Analysis: Metabolism-Blood Chemistry

In the area of metabolism, targets may be identified for the treatment of diabetes. Blood chemistry phenotypic analysis includes blood glucose measurements. The COBAS Integra 400 (mfr: Roche) was used for running blood chemistry tests on the mice. In addition to measuring blood glucose levels the following blood chemistry tests are also routinely performed: Alkaline Phosphatase; Alanine Amino-Transferase; Albumin; Bilirubin; Phosphorous; Creatinine; BUN=Blood Urea Nitrogen; Calcium; Uric Acid; Sodium; Potassium; and Chloride. In the area of metabolism, targets may be identified for the treatment of diabetes.

Results:

The male (−/−) mice exhibited decreased mean serum glucose levels when compared with their gender-matched (+/+) littermates and the historical means. However, the (−/−) mice had a normal Glucose Tolerance.

70.54. Generation and Analysis of Mice Comprising DNA105838-2702 (UNQ2528) Gene Disruptions

In these knockout experiments, the gene encoding PRO6027 polypeptides (designated as DNA105838-2702) (UNQ2528) was disrupted. The gene specific information for these studies is as follows: the mutated mouse gene corresponds to nucleotide reference: NM_{—}027476 Mus musculus leukocyte receptor cluster (LRC) member 4 (Leng4); protein reference: Q6IR37ACCESSION:Q61R37NID: Mus musculus (Mouse). Leng4 protein; the human gene sequence reference:NM207340 Homo sapiens hypothetical protein LOC254359 (LOC254359); the human protein sequence corresponds to reference: Q6UX98 ACCESSION:Q6UX98 NID: Homo sapiens (Human). LENG4.

The mouse gene of interest is Leng4 (leukocyte receptor cluster [LRC] member 4), ortholog of human hypothetical protein LOC254359. Aliases include 5730496N17Rik.

Leng4 is a likely integral membrane protein, consisting of three transmembrane segments and a DHHC zinc finger domain (Pfam accession PF01529). The predicted subcellular location of Leng4 is ambiguous; it may be located on the plasma membrane or in mitochondria. Proteins with DHHC zinc finger domains include putative transcription factor DNZ1 from D. melanogaster (Mesilaty-Gross et al, Gene 231(1-2):173-86 (1999)) and noncatalytic palmitoyltransferase subunit ERF2 from S. cerevisiae, which is located on the membrane of the endoplasmic reticulum (Lobo et al, J Biol Chem 277(43):41268-73 (2002)). DHHC zinc finger domain has been predicted to be involved in protein-protein or protein-DNA interactions (Putilina et al, Mol Cell Biochem 195(1-2):219-26 (1999)). The function of Leng4 is not known.

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 18 | 47 | 16 | 81 |
| Expected | 20.25 | 40.5 | 20.25 | 81 |

Chi-Sq.=4.83 Significance=0.089367345 (hom/n)=0.19 Avg. Litter Size=9

### Mutation Information

Mutation Type: Retroviral Insertion (OST)
Description: Retroviral insertion occurred in the intron between coding exons 1 and 2 (NCBI accession NM_{—}027476.1).
1. Wild-type Expression Panel: Expression of the target gene was detected in embryonic stem (ES) cells and in all 13 adult tissue samples tested by RT-PCR, except eye, liver, skeletal muscle, bone, heart, and adipose.
2. QC Expression: RT-PCR analysis revealed that the transcript was absent in the (−/−) mouse analyzed (M-104). Disruption of the target gene was confirmed by Inverse PCR.

70.54.1. Phenotypic Analysis (for Disrupted Gene: DNA105838-2702 (UNQ2528)

(a) Overall Phenotypic Summary:

Mutation of the gene encoding the ortholog of a human hypothetical protein (LOC254359) resulted in anemia in the homozygous mutant mice. The (−/−) mice also exhibited increased mean femoral mid-shaft cortical thickness. Transcript was absent by RT-PCR.

(b) Immunology Phenotypic Analysis

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

T lymphocytes (T cells) are an important component of a mammalian immune response. T cells recognize antigens which are associated with a self-molecule encoded by genes within the major histocompatibility complex (MHC). The antigen may be displayed together with MHC molecules on the surface of antigen presenting cells, virus infected cells, cancer cells, grafts, etc. The T cell system eliminates these altered cells which pose a health threat to the host mammal. T cells include helper T cells and cytotoxic T cells. Helper T cells proliferate extensively following recognition of an antigen-MHC complex on an antigen presenting cell. Helper T cells also secrete a variety of cytokines, i.e., lymphokines, which play a central role in the activation of B cells, cytotoxic T cells and a variety of other cells which participate in the immune response.

In many immune responses, inflammatory cells infiltrate the site of injury or infection. The migrating cells may be neutrophilic, eosinophilic, monocytic or lymphocytic as can be determined by histologic examination of the affected tissues. Current Protocols in Immunology, ed. John E. Coligan, 1994, John Wiley & Sons, Inc.

Many immune related diseases are known and have been extensively studied. Such diseases include immune-mediated inflammatory diseases (such as rheumatoid arthritis, immune mediated renal disease, hepatobiliary diseases, inflammatory bowel disease (IBD), psoriasis, and asthma), non-immune-mediated inflammatory diseases, infectious diseases, immunodeficiency diseases, neoplasia, and graft rejection, etc. In the area of immunology, targets were identified for the treatment of inflammation and inflammatory disorders.

In the area of immunology, targets have been identified herein for the treatment of inflammation and inflammatory disorders. Immune related diseases, in one instance, could be treated by suppressing the immune response. Using neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

The following tests were performed:

Hematology Analysis:

Test Description: Blood tests are carried out by Abbott's Cell-Dyn 3500R, an automated hematology analyzer. Some of its features include a five-part WBC differential. ‘Patient’ reports can cover over 22 parameters in all.

Results:

The (−/−) mice exhibited a decreased mean total red blood cell count, hemoglobin level, and hematocrit levels when compared with their (+/+) littermates and the historical means.

The mutant (−/−) mice exhibited a phenotype associated with anemia. Thus, PRO6027 polypeptides, agonists thereof or the encoding gene for PRO6027 polypeptides must be essential for normal red blood cell production and as such would be useful in the treatment of blood disorders associated with anemia or a low hematocrit.

(c) Bone Metabolism & Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Bone microCT Analysis:

Procedure: MicroCT was also used to get very sensitive measurements of BMD. One vertebra and 1 femur were taken from a cohort of 4 wild type and 8 homozygous mice. Measurements were taken of lumbar 5 vertebra trabecular bone volume, trabecular thickness, connectivity density and midshaft femur total bone area and cortical thickness. The μCT40 scans provided detailed information on bone mass and architecture. Multiple bones were placed into sample holders and scanned automatically. Instrument software was used to select regions of interest for analysis. Trabecular bone parameters were analyzed in the fifth lumbar vertebrae (LV5) at 16 micrometer resolution and cortical bone parameters were analyzed in the femur midshaft at a resolution of 20 micrometers.

Results:

The male (−/−) mice exhibited increased mean femoral mid-shaft cortical thickness when compared with their gender-matched (+/+) littermates and the historical mean.

In summary, the (−/−) mice exhibited increased femoral mid-shaft cortical thickness when compared with their gender-matched (+/+) littermates. These results indicate that the knockout mutant phenotype is associated with such bone abnormalities as osteopetrosis. Osteopetrosis is a condition characterized by abnormal thickening and hardening of bone and abnormal fragility of the bones. As such, PRO6027 polypeptides or agonists thereof would be beneficial for the treatment of osteopetrosis or other osteo-related diseases. On the other hand, inhibitors or antagonists of PRO6027 polypeptides would be useful in bone healing.

70.55. Generation and Analysis of Mice Comprising DNA 107698-2715 (UNQ2552) Gene Disruptions

In these knockout experiments, the gene encoding PRO6181 polypeptides (designated as DNA107698-2715) (UNQ2552) was disrupted. The gene specific information for these studies is as follows: the mutated mouse gene corresponds to nucleotide reference: NM_{—}182785 Mus musculus RIKEN cDNA 4933400F01 gene (4933400F01Rik); protein reference: Q8BVP6 ACCESSION:Q8BVP6 NID: Mus musculus (Mouse). Mus musculus adult male testis cDNA, RIKEN full-length enriched library, clone:4933400F01 product:hypothetical Serine proteases, trypsin family containing protein, full insert sequence (Hypothetical protein 4933400F01Rik); the human gene sequence reference: NM_{—}173506 Homo sapiens hypothetical protein MGC42718 (MGC42718); the human protein sequence corresponds to reference: Q6UWNO ACCESSION:Q6UWNO NID: Homo sapiens (Human). GPQH2552.

The mouse gene of interest is RIKEN cDNA 4933400F01 gene, ortholog of human hypothetical protein MGC42718. Aliases include MGC58778 and MGC42718.

Hypothetical protein MGC42718 is a putative secreted protein of about 250 amino acids, containing a signal peptide. No other conserved domains were detected.

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 25 | 42 | 21 | 88 |
| Expected | 22 | 44 | 22 | 88 |

Chi-Sq.=0.04 Significance=0.9801987 (hom/n)=0.25 Avg. Litter Size=9

### Mutation Information

Mutation Type: Homologous Recombination (standard)
Description: Coding exons 1 through 3 were targeted (NCBI accession NM_{—}182785.1).
1. Wild-type Expression Panel: Expression ofthe target gene was detected in brain, eye, thymus, spleen, lung, and adipose among the 13 adult tissue samples tested by RT-PCR.
2. QC Expression: Disruption of the target gene was confirmed by Southern hybridization analysis.

70.55.1. Phenotypic Analysis (for Disrupted Gene: DNA107698-2715 (UNQ2552)

(a) Overall Phenotypic Summary:

Mutation of the gene encoding the ortholog of a human hypothetical protein MGC42718 resulted in an increased IgG2a response to ovalbumin challenge in the (−/−) mice. The (−/−) mice also exhibited decreased total tissue mass and total percent body fat (%) and fat mass (g). Gene disruption was confirmed by Southern blot.

(b) Immunology Phenotypic Analysis

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

T lymphocytes (T cells) are an important component of a mammalian immune response. T cells recognize antigens which are associated with a self-molecule encoded by genes within the major histocompatibility complex (MHC). The antigen may be displayed together with MHC molecules on the surface of antigen presenting cells, virus infected cells, cancer cells, grafts, etc. The T cell system eliminates these altered cells which pose a health threat to the host mammal. T cells include helper T cells and cytotoxic T cells. Helper T cells proliferate extensively following recognition of an antigen-MHC complex on an antigen presenting cell. Helper T cells also secrete a variety of cytokines, i.e., lymphokines, which play a central role in the activation of B cells, cytotoxic T cells and a variety of other cells which participate in the immune response.

In many immune responses, inflammatory cells infiltrate the site of injury or infection. The migrating cells may be neutrophilic, eosinophilic, monocytic or lymphocytic as can be determined by histologic examination of the affected tissues. Current Protocols in Immunology, ed. John E. Coligan, 1994, John Wiley & Sons, Inc.

Many immune related diseases are known and have been extensively studied. Such diseases include immune-mediated inflammatory diseases (such as rheumatoid arthritis, immune mediated renal disease, hepatobiliary diseases, inflammatory bowel disease (IBD), psoriasis, and asthma), non-immune-mediated inflammatory diseases, infectious diseases, immunodeficiency diseases, neoplasia, and graft rejection, etc. In the area of immunology, targets were identified for the treatment of inflammation and inflammatory disorders.

In the area of immunology, targets have been identified herein for the treatment of inflammation and inflammatory disorders. Immune related diseases, in one instance, could be treated by suppressing the immune response. Using neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

The following test was performed:

Ovalbumin Challenge

Procedure: This assay was carried out on 7 wild type and 8 homozygous mice. Chicken ovalbumin (OVA) is a T-cell dependent antigen, which is commonly used as a model protein for studying antigen-specific immune responses in mice. OVA is non-toxic and inert and therefore will not cause harm to the animals even if no immune response is induced. The murine immune response to OVA has been well characterized, to the extent that the immunodominant peptides for eliciting T cell responses have been identified. Anti-OVA antibodies are detectable 8 to 10 days after immunization using enzyme-linked immunosorbent assay (ELIZA), and determination of different isotypes of antibodies gives further information on the complex processes that may lead to a deficient response in genetically engineered mice.

As noted above, this protocol assesses the ability of mice to raise an antigen-specific immune response. Animals were injected IP with 50 mg of chicken ovalbumin emulsified in Complete Feund's Adjuvant and 14 days later the serum titer of anti-ovalbumin antibodies (IgM, IgG1 and IgG2 subclasses) was measured. The amount of OVA-specific antibody in the serum sample is proportional to the Optical Density (OD) value generated by an instrument that scans a 96-well sample plate. Data was collected for a set of serial dilutions of each serum sample.

Results of this Challenge:

The (−/−) mice exhibited an increased mean serum IgG2a response to ovalbumin challenge when compared with their (+/+) littermates and the historical mean.

In summary, the ovalbumin challenge studies indicate that knockout mice deficient in the gene encoding PRO6181 polypeptides exhibit immunological abnormalities when compared with their wild-type littermates. In particular, the mutant mice exhibited an increased ability to elicit an immunological response when challenged with the T-cell dependent OVA antigen. Thus, antagonists (inhibitors) of PRO6181 polypeptides would be useful for stimulating the immune system (such as T cell proliferation) and would find utility in the cases wherein this effect would be beneficial to the individual such as in the case of leukemia, and other types of cancer, and in immunocompromised patients, such as AIDS sufferers. Accordingly, PRO6181 polypeptides or agonists thereof, would be useful for inhibiting the immune response and thus would be useful candidates for suppressing harmful immune responses, e.g. in the case of graft rejection or graft-versus-host diseases.

(c) Bone Metabolism & Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Results:

Female (−/−) mice exhibited a decreased total tissue mass, mean total body fat percent (%) and total mean body fat mass(g) when compared with their gender-matched wildtype littermates and the historic means.

These studies suggest that mutant (−/−) non-human transgenic animals exhibit a negative phenotype that would be associated with tissue wasting diseases. Thus, PRO6181 polypeptides or agonists thereof are essential for normal growth and metabolic processes.

70.56. Generation and Analysis of Mice Comprising DNA82358-2738 (UNQ2759) Gene Disruptions

In these knockout experiments, the gene encoding PRO6714 polypeptides (designated as DNA82358-2738) (UNQ2759) was disrupted. The gene specific information for these studies is as follows: the mutated mouse gene corresponds to nucleotide reference: NM_{—}023158 ACCESSION:NMO23158 NID: gi 14249120 ref NMO23158.2 Mus musculus Cxc chemokine ligand 16 (Cxcll 6); protein reference: Q9EPB3 ACCESSION:Q9EPB3 NID: Mus musculus (Mouse). SR—PSOX (Transmembrane chemokine CXCL16) (0910001 K24RIK protein) (CXC chemokine ligand 16); the human gene sequence reference: NMO22059 Homo sapiens chemokine (C—X—C motif) ligand 16 (CXCL16); the human protein sequence corresponds to reference: Q9H2A7 ACCESSION:Q9H2A7 NID: Homo sapiens (Human). Small inducible cytokine B16 (Transmembrane chemokine CXCL16) (SR-PSOX) (Scavenger receptor for phosphatidylserine and oxidized low density lipoprotein).

The mouse gene of interest is Cxcl16 (chemokine [C—X—C motif] ligand 16), ortholog of human CXCL16. Aliases include SR—PSOX, SRPSOX, CXCLG16, AV290116, 0910001K24Rik, SR-PSOX/CXCL16, and Cxc chemokine ligand 16.

CXCL16 is a type I integral plasma membrane protein that functions as a chemokine ligand for G protein-coupled receptor CXCR6 (chemokine [C—X—C motif] receptor 6) (Matloubian et al, Nat Immunol 1(4):298-304 (2000)). In macrophages, CXCL16 also functions as a scavenger receptor for oxidized low-density lipoprotein (Shimaoka et al, J Biol Chem 275(52):40663-6 (2000)). CXCL16 on macrophages is rapidly shed in vitro in response to inflammatory mediators, such as tumor necrosis factor-alpha or lipopolysaccharides, suggesting that the protein is also located extracellularly (Wilbanks et al, 2001).

CXCL16 is expressed primarily in Peyer patches, lung, kidney, small intestine, splenic red pulp, and T-cell areas of splenic white pulp, lymph nodes, and thymus medulla. CXCL16 expression has been detected on a variety of immune cells, including dendritic cells, B-cells, monocytes, and macrophages (Matloubian et al, Nat Immunol 1(4):298-304 (2000); Shimaoka et al, J Biol Chem 275(52):40663-6 (2000)). CXCL16 expression generally increases in response to inflammatory stimuli, and CXCL16 induces strong chemotaxis of T cells. Thus, CXCL16 is likely to be involved in T-cell migration, immune cell interactions, immune cell adhesion, phagocytosis, and oxidized low-density lipoprotein endocytosis in macrophages (Wilbanks et al, J Immunol 166(8):5145-54 (2001); Chandrasekar et al, J Biol Chem 279(5):3188-96 (2004); Shimaoka et al, J Leukoc Biol 75(2):267-74 (2004); Shimaoka et al, J Immunol 171(4):1647-51 (2003)). Moreover, CXCL16 may play a role in inflammatory diseases, such as autoimmune encephalomyelitis and atherosclerosis (Fukumoto et al, J Immunol 173(3):1620-7 (2004); Chandrasekar et al, J Biol Chem 279(5):3188-96 (2004); Yamauchi et al, Arterioscler Thromb Vasc Biol 24(2):282-7 (2004); Wuttge et al, Arterioscler Thromb Vasc Biol 24(4):750-5 (2004)).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 20 | 37 | 20 | 77 |
| Expected | 19.25 | 38.5 | 19.25 | 77 |

Chi-Sq.=1.43 Significance=0.48919213 (hom/n)=0.23 Avg. Litter Size=8

### Mutation Information

Mutation Type: Homologous Recombination (standard)
Description: Coding exons 1 through 3 were targeted (NCBI accession NM_{—}023158.3).
1. Wild-type Expression Panel: Expression of the target gene was detected in embryonic stem (ES) cells and in all 13 adult tissue samples tested by RT-PCR, except bone.
2. QC Expression: Disruption of the target gene was confirmed by Southern hybridization analysis.

70.56.1. Phenotypic Analysis (for Disrupted Gene: DNA82358-2738 (UNQ2759)

(a) Overall Phenotypic Summary:

Mutation of the gene encoding the ortholog of human chemokine (C—X—C motif) ligand 16 (CXCL16) resulted in hyperactivity in the (−/−) mice during neurological testing. Gene disruption was confirmed by Southern blot.

(b) Phenotypic Analysis: CNS/Neurology

In the area of neurology, analysis focused herein on identifying in vivo validated targets for the treatment of neurological and psychiatric disorders including depression, generalized anxiety disorders, attention deficit hyperactivity disorder, obsessive compulsive disorder, schizophrenia, cognitive disorders, hyperalgesia and sensory disorders. Neurological disorders include the category defined as “anxiety disorders” which include but are not limited to: mild to moderate anxiety, anxiety disorder due to a general medical condition, anxiety disorder not otherwise specified, generalized anxiety disorder, panic attack, panic disorder with agoraphobia, panic disorder without agoraphobia, posttraumatic stress disorder, social phobia, specific phobia, substance-induced anxiety disorder, acute alcohol withdrawal, obsessive compulsive disorder, agoraphobia, bipolar disorder I or II, bipolar disorder not otherwise specified, cyclothymic disorder, depressive disorder, major depressive disorder, mood disorder, substance-induced mood disorder. In addition, anxiety disorders may apply to personality disorders including but not limited to the following types: paranoid, antisocial, avoidant behavior, borderline personality disorders, dependent, histronic, narcissistic, obsessive-compulsive, schizoid, and schizotypal.

Procedure:

Behavioral screens were performed on a cohort of 4 wild type, 4 heterozygous and 8 homozygous mice. All behavioral tests were done between 12 and 16 weeks of age unless reduced viability necessitates earlier testing. These tests included open field to measure anxiety, activity levels and exploration.

Circadian Test Description:

Female mice are individually housed at 4 pm on the first day of testing in 48.2 cm×26.5 cm home cages and administered food and water ad libitum. Animals are exposed to a 12-hour light/dark cycle with lights turning on at 7 am and turning off at 7 pm. The system software records the number of beam interruptions caused by the animal's movements, with beam breaks automatically divided into ambulations. Activity is recorded in 60, one-hour intervals during the three-day test. Data generated are displayed by median activity levels recorded for each hour (circadian rhythm) and median total activity during each light/dark cycle (locomotor activity) over the three-day testing period.

Results:

The female (−/−) mice exhibited increased ambulatory counts during both dark periods of home-cage activity testing resulting in a hyperactive behavior pattern when compared with their gender-matched (+/+) littermates and the historical means, suggestive of increased anxiety which is consistent with neurological disorders such as generalized anxiety disorders, attention deficit hyperactivity disorder, obsessive compulsive disorder, schizophrenia, cognitive disorders generalized anxiety disorder. Thus, PRO6714 polypeptides would be useful in treating such disorders.

70.57. Generation and Analysis of Mice Comprising DNA142524 (UNQ2768) Gene Disruptions

In these knockout experiments, the gene encoding PRO9922 polypeptides (designated as DNA142524) (UNQ2768) was disrupted. The gene specific information for these studies is as follows: the mutated mouse gene corresponds to nucleotide reference: NM_{—}126166 Mus musculus toll-like receptor 3 (Th-3); protein reference: Q99MB1 Q99 MB1 Q99 MB1 TOLL-LIKE RECEPTOR3; the human gene sequence reference: NM_{—}003265 ACCESSION:NM003265 NID:19718735 Homo sapiens Homo sapiens toll-like receptor 3 (TLR3); the human protein sequence corresponds to reference: O15455 O15455 O15455 TOLL-LIKE RECEPTOR 3.

The mouse gene of interest is Th-3 (toll-like receptor 3), ortholog of human TLR3. TLR3 is a type I plasma membrane protein that functions as a pattern recognition receptor, recognizing double-stranded RNA and other microbial agents (Alexopoulou et al, Nature 413(6857):732-8 (2001); Brightbill et al, Science 285(5428):732-6 (1999); Rock et al, Proc Natl Acad Sci USA 95(2):588-93 (1998)). TLR3 is expressed in placenta, pancreas (Rock et al, Proc Natl Acad Sci USA 95(2):588-93 (1998)), dendritic cells (Muzio et al, J Immunol 164(11):5998-6004 (2000), and osteoclast precursors (Takami et al, J Immunol 169(3):1516-23 (2002). Activation of TLR3 results in production of the powerful microbicide nitric oxide (Brightbill et al, Science 285(5428):732-6 (1999)) and inflammatory cytokines (Kariko et al, J Immunol 172(11):6545-9 (2004); Alexopoulou et al, Nature 413(6857):732-8 (2001)). TLR3 is involved in innate and adaptive immunity (Heinz et al, J Biol Chem 278(24):21502-9 (2003); Olson and Miller, J Immunol 173(6):3916-24 (2004); Applequist et al, Int Immunol 14(9):1065-74 (2002)).

Alexopoulou and colleagues [Nature 413 (6857): 732-8 (2001)] investigated the physiological role of TLR3 using knockout mice. They found that inflammation and inflammatory cytokine production in response to double-stranded RNA were substantially lower in TLR3-deficient mice than in wild-type mice. Moreover, they showed that the response to double-stranded RNA in TLR3-deficient mice sensitized with D-galactosamine was less lethal than that in wild-type mice sensitized with D-galactosamine. They concluded that the ability of TLR3 to recognize double-stranded RNA provides innate immunity against viral infection.

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 27 | 38 | 16 | 81 |
| Expected | 20.25 | 40.5 | 20.25 | 81 |

Chi-Sq.=13.84 Significance=9.878299E-4 (hom/n)=0.19 Avg. Litter Size=9

### Mutation Information

Mutation Type: Homologous Recombination (standard)
Description: Coding exons 1 and 2 were targeted (NCBI accession NM_{—}126 166.2).
1. Wild-type Expression Panel: Expression of the target gene was detected in embryonic stem (ES) cells and in all 13 adult tissue samples tested by RT-PCR, except bone.
2. QC Expression: Disruption of the target gene was confirmed by Southern hybridization analysis.

70.57.1. Phenotypic Analysis (for Disrupted Gene: DNA142524 (UNQ2768)

(a) Overall Phenotypic Summary:

Mutation of the gene encoding the ortholog of human toll-like receptor 3 (TLR3) resulted in decreased vertebral bone-related measurements. Gene disruption was confirmed by Southern blot.

(b) Bone Metabolism & Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Bone MicroCT Analysis:

Procedure: MicroCT was also used to get very sensitive measurements of BMD. One vertebra and 1 femur were taken from a cohort of 4 wild type and 8 homozygous mice. Measurements were taken of lumbar 5 vertebra trabecular bone volume, trabecular thickness, connectivity density and midshaft femur total bone area and cortical thickness. The μCT40 scans provided detailed information on bone mass and architecture. Multiple bones were placed into sample holders and scanned automatically. Instrument software was used to select regions of interest for analysis. Trabecular bone parameters were analyzed in the fifth lumbar vertebrae (LV5) at 16 micrometer resolution and cortical bone parameters were analyzed in the femur midshaft at a resolution of 20 micrometers.

Results:

DEXA: The male (−/−) mice exhibited decreased mean vertebral bone mineral density when compared with their gender-matched (+/+) littermates and the historical mean. Female (−/−) mice exhibited a decreased BMC/LBM index compared with their gender-matched wildtype littermates.
Micro-CT: The male (−/−) mice exhibited decreased mean vertebral trabecular bone volume, number, thickness, and connectivity density and decreased mean femoral midshaft cross-sectional area when compared with their gender-matched (+/+) littermates and the historical means.

The (−/−) mice analyzed by DEXA and bone micro CT analysis exhibited decreased vertebral bone measurements when compared with their (+/+) littermates, suggestive of abnormal bone disorders. The (−/−) mice exhibited a negative bone phenotype with abnormal decreased bone measurements reflective of bone metabolic disorders. The negative bone phenotype indicates that PRO9922 polypeptides or agonists thereof would be useful for maintaining bone homeostasis. In addition, PRO9922 polypeptides would be useful in bone healing or for the treatment of arthritis or osteoporosis, whereas antagonists (or inhibitors) of PRO9922 polypeptides or its encoding gene would lead to abnormal or pathological bone disorders including inflammatory diseases associated with abnormal bone metabolism including arthritis, osteoporosis and osteopenia.

70.58. Generation and Analysis of Mice Comprising DNA108701-2749 (UNQ2789) Gene Disruptions

In these knockout experiments, the gene encoding PRO7179 polypeptides (designated as DNA108701-2749) (UNQ2789) was disrupted. The gene specific information for these studies is as follows: the mutated mouse gene corresponds to nucleotide reference: NM_{—}010584 ACCE SSION:NM_{—}010584 NID: 6754387 Mus musculus Mus musculus intelectin (Itln); protein reference: 088310 ACCESSION:088310 NID: Mus musculus (Mouse). INTELECTIN (10 DAY OLD MALE PANCREAS cDNA, RIKEN FULL-LENGTH ENRICHED LIBRARY, CLONE:1810012B21, FULL INSERT SEQUENCE); the human gene sequence reference: NM_{—}017625 ACCESSION:NM_{—}017625 NID:8923027 Homo sapiens Homo sapiens intelectin (ITLN); the human protein sequence corresponds to reference: Q9NP67 ACCESSION:Q9NP67NID: Homo sapiens (Human). INTELECTIN (cDNA FLJ20022 FIS, CLONE ADSE01331) (ENDOTHELIAL LECTIN HL-1).

The mouse gene of interest is Itln (intelectin), ortholog of human ITLN1 (intelectin 1 [galactofuranose binding]). Aliases include IntL, LFR, HL-1, ITLN, hIntL, FLJ20022, endothelial lectin HL-1, and intestinal lactoferrin receptor.

ITLN1 is a glycosyl-phosphatidylinositol-anchored extracellular protein that likely functions as a receptor for lactoferrin and a secreted lectin-like protein that binds with galactofuranosyl residues in bacterial cell walls. The protein is expressed in small intestine, colon, heart, and thymus. In newborn infants, ITLN1 is likely to be expressed on the surface of small intestine epithelium, where it plays a role in the uptake of lactoferrin, a major form of iron in milk ITLN1 may also function in innate immunity by binding with microorganisms (Suzuki et al, Biochemistry 40(51):15771-9 (2001); Komiya et al, Biochem Biophys Res Commun 251(3):759-62 (1998); Tsuji et al, J Biol Chem 276(26):23456-63 (2001)).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 24 | 40 | 10 | 74 |
| Expected | 18.5 | 37 | 18.5 | 74 |

Chi-Sq.=9.16 Significance=0.010254897 (hom/n)=0.16 Avg. Litter Size=7

### Mutation Information

Mutation Type: Homologous Recombination (standard)
Description: Coding exons 1 through 4 were targeted (NCBI accession NM_{—}010584.1).
1. Wild-type Expression Panel: Expression of the target gene was detected only in stomach, small intestine, and colon among the 13 adult tissue samples tested by RT-PCR.
2. QC Expression: Disruption of the target gene was confirmed by Southern hybridization analysis.

70.58.1. Phenotypic Analysis (for disrupted gene: DNA108701-2749 (UNQ2789)

(a) Overall Phenotypic Summary:

Mutation of the gene encoding the ortholog of human intelectin 1 (galactofuranose binding) (ITLN1) resulted in decreased bone mineral content and density measurements in the (−/−) mice. Gene disruption was confirmed by Southern blot.

(b) Bone Metabolism & Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Bone MicroCT Analysis:

Procedure: MicroCT was also used to get very sensitive measurements of BMD. One vertebra and 1 femur were taken from a cohort of 4 wild type and 8 homozygous mice. Measurements were taken of lumbar 5 vertebra trabecular bone volume, trabecular thickness, connectivity density and midshaft femur total bone area and cortical thickness. The μCT40 scans provided detailed information on bone mass and architecture. Multiple bones were placed into sample holders and scanned automatically. Instrument software was used to select regions of interest for analysis. Trabecular bone parameters were analyzed in the fifth lumbar vertebrae (LV5) at 16 micrometer resolution and cortical bone parameters were analyzed in the femur midshaft at a resolution of 20 micrometers.

Results:

DEXA: The male (−/−) mice exhibited decreased bone mineral content, decreased BMC/LBM index, decreased femur bone mineral density and decreased vertebrae bone mineral density when compared with their gender-matched (+/+) littermates and the historical mean.
MicroCT: The male (−/−) mice exhibited decreased vertebral trabecular bone volume, number, and thickness and decreased mean femoral mid-shaft cross-sectional area when compared with their gender-matched wildtype littermates and the historical means.

The (−/−) mice analyzed by DEXA and bone micro CT analysis exhibited decreased bone measurements when compared with their (+/+) littermates, suggestive of abnormal bone disorders. The (−/−) mice exhibited a negative bone phenotype with abnormal decreased bone measurements reflective of bone metabolic disorders. The negative bone phenotype indicates that PRO7179 polypeptides or agonists thereof would be useful for maintaining bone homeostasis. In addition, PRO7179 polypeptides would be useful in bone healing or for the treatment of arthritis or osteoporosis, whereas antagonists (or inhibitors) of PRO7179 polypeptides or its encoding gene would lead to abnormal or pathological bone disorders including inflammatory diseases associated with abnormal bone metabolism including arthritis, osteoporosis and osteopenia.

70.59. Generation and Analysis of Mice Comprising DNA115253-2757 (UNQ2976) Gene Disruptions

In these knockout experiments, the gene encoding PRO7476 polypeptides (designated as DNA115253-2757) (UNQ2976) was disrupted. The gene specific information for these studies is as follows: the mutated mouse gene corresponds to nucleotide reference: NMO24449 ACCESSION:NMO24449 NID:14209687 Mus musculus Mus musculus sclerostin (Sost); protein reference:Q99P68 ACCESSION:Q99P68 NID: Mus musculus (Mouse). SCLEROSTIN PRECURSOR; the human gene sequence reference: NM_{—}025237 ACCESSION:NM_{—}025237 NID:13376845 Homo sapiens Homo sapiens sclerosteosis (SOST); the human protein sequence corresponds to reference: Q9BQB4 ACCESSION:Q9BQB4 NID: Homo sapiens (Human). SCLEROSTIN PRECURSOR.

The mouse gene of interest is Sost (sclerostin), ortholog of human SOST. Aliases include 5430411E23Rik.

SOST is a secreted glycoprotein expressed primarily in osteocytes that binds with bone morphogenic protein receptors, antagonizing receptor activation by bone morphogenic protein. SOST is involved in bone homeostasis, suppressing mineralization of osteoblastic cells. Loss-of-function mutations in the SOST gene cause sclerosteosis, an autosomal recessive disorder characterized by skeletal overgrowth (Brunkow et al, Am J Hum Genet. 68(3):577-89 (2001); Balemans et al, Hum Mol Genet. 10(5):537-43 (2001); Winkler et al, EMBO J. 22(23):6267-76 (2003); Kusu et al, J Biol Chem 278(26):24113-7 (2003); Van Bezooijen et al, J Exp Med 199(6):805-14 (2004)).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 19 | 25 | 24 | 68 |
| Expected | 17 | 34 | 17 | 68 |

Chi-Sq.=0.96 Significance=0.6187834 (hom/n)=0.25 Avg. Litter Size=8

### Mutation Information

Mutation Type: Homologous Recombination (standard)
Description: Coding exons 1 and 2 were targeted (NCBI accession AK017295.1).
1. Wild-type Expression Panel: Expression of the target gene was detected, among 13 adult tissue samples tested by RT-PCR, in brain, eye, thymus, spleen, lung, kidney, and heart.
2. QC Expression: Disruption of the target gene was confirmed by Southern hybridization analysis.

70.59.1. Phenotypic Analysis (for Disrupted Gene: DNA115253-2757 (UNQ2976)

(a) Overall Phenotypic Summary:

Mutation of the gene encoding the ortholog of human sclerosteosis (SOST) resulted in osteopetrosis in (−/−) mice. The (−/−) mice also exhibited blood chemistry and hematological abnormalities. The homozygous mutant mice exhibited diffuse moderate osteopetrosis, characterized by diffuse thickening of the trabecular bone and increased bone mineral content and density measurements. In addition, the (−/−) mice exhibited an increased mean serum IgG2b level and decreased red blood cell count and decreased mean corpuscular volume. Disruption of the target gene was confirmed by Southern hybridization analysis.

(b) Pathology:

Gross Observations: Among the 6 (−/−) mice analyzed, 3 exhibited thickened bone.
Microscopic Observations All 6 (−/−) mice exhibited diffuse moderate osteopetrosis, characterized by diffuse thickening of trabecular bone in the femur, sternum, vertebra, and nasal turbinates and maxilla of the head. Bone surfaces were fully covered by osteoblasts, and only small numbers of osteoclasts were present. The lesions in these mutants were similar to those seen in human patients with sclerosteosis.
Gene Expression: LacZ activity was not detected in the panel of tissues by immunohistochemical analysis.

(c) Immunology Phenotypic Analysis

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

T lymphocytes (T cells) are an important component of a mammalian immune response. T cells recognize antigens which are associated with a self-molecule encoded by genes within the major histocompatibility complex (MHC). The antigen may be displayed together with MHC molecules on the surface of antigen presenting cells, virus infected cells, cancer cells, grafts, etc. The T cell system eliminates these altered cells which pose a health threat to the host mammal. T cells include helper T cells and cytotoxic T cells. Helper T cells proliferate extensively following recognition of an antigen-MHC complex on an antigen presenting cell. Helper T cells also secrete a variety of cytokines, i.e., lymphokines, which play a central role in the activation of B cells, cytotoxic T cells and a variety of other cells which participate in the immune response.

In many immune responses, inflammatory cells infiltrate the site of injury or infection. The migrating cells may be neutrophilic, eosinophilic, monocytic or lymphocytic as can be determined by histologic examination of the affected tissues. Current Protocols in Immunology, ed. John E. Coligan, 1994, John Wiley & Sons, Inc.

Many immune related diseases are known and have been extensively studied. Such diseases include immune-mediated inflammatory diseases (such as rheumatoid arthritis, immune mediated renal disease, hepatobiliary diseases, inflammatory bowel disease (IBD), psoriasis, and asthma), non-immune-mediated inflammatory diseases, infectious diseases, immunodeficiency diseases, neoplasia, and graft rejection, etc. In the area of immunology, targets were identified for the treatment of inflammation and inflammatory disorders.

In the area of immunology, targets have been identified herein for the treatment of inflammation and inflammatory disorders. Immune related diseases, in one instance, could be treated by suppressing the immune response. Using neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

The following test was performed:

Serum Immunoglobulin Isotyping Assay:

The Serum Immunoglobulin Isotyping Assay is performed using a Cytometric Bead Array (CBA) kit. This assay is used to rapidly identify the heavy and light chain isotypes of a mouse monoclonal antibody in a single sample. The values expressed are “relative fluorescence units” and are based on the detection of kappa light chains. Any value <6 is not significant.

Results:

Serum Immunoglobulins: The (−/−) mice exhibited an increased mean serum IgG2b level when compared with that of their (+/+) littermates, the (+/+) mice within the project run, and the historical median.

Thus, homozygotes showed an elevation of mean serum immunoglobulins compared with the (+/+) littermates. IgG2b immunoglobulins have neutralization effects and to a lesser extent are important for activation of the complement system. These immunological abnormalities suggest that antagonists or inhibitors of PRO7476 polypeptides would stimulate the immune system (such as T cell proliferation) and would find utility in the cases wherein this effect would be beneficial to the individual such as in the case of leukemia, and other types of cancer, and in immunocompromised patients, such as AIDS sufferers. Accordingly, PRO7476 polypeptides or agonists thereof would inhibit the immune response and would be useful candidates for suppressing harmful immune responses, e.g. in the case of graft rejection or graft-versus-host diseases.

Hematology Analysis:

Test Description: Blood tests are carried out by Abbott's Cell-Dyn 3500R, an automated hematology analyzer. Some of its features include a five-part WBC differential. ‘Patient’ reports can cover over 22 parameters in all.

Results:

Hematology: The (−/−) mice exhibited a decreased mean red blood cell count and an increased mean corpuscular volume when compared with their (+/+) littermates and the historical means.

In addition to the observation of increased serum IgG2b immunoglobulins, the mutant (−/−) mice exhibited a phenotype associated with anemia. Thus, PRO7476 polypeptides, agonists thereof or the encoding gene for PRO7476 polypeptides must be essential for normal red blood cell production and as such would be useful in the treatment of blood disorders associated with anemia or a low hematocrit.

(d) Phenotypic Analysis: Cardiology

In the area of cardiovascular biology, targets were identified herein for the treatment of hypertension, atherosclerosis, heart failure, stroke, various coronary artery diseases, dyslipidemias such as high cholesterol (hypercholesterolemia) and elevated serum triglycerides (hypertriglyceridemia), diabetes and/or obesity. The phenotypic tests included the measurement of serum cholesterol and triglycerides.

Blood Lipids

Procedure: A cohort of 4 wild type, 4 heterozygous and 8 homozygous mice were tested in this assay. High cholesterol levels and increased triglyceride blood levels are recognized risk factors in the development of cardiovascular disease and/or diabetes. Measuring blood lipids facilitates the finding of biological switches that regulate blood lipid levels Inhibition of factors which elevate blood lipid levels may be useful for reducing the risk for cardiovascular disease. In these blood chemistry tests, measurements were recorded using the COBAS Integra 400 (mfr: Roche).

Results:

The (−/−) mice exhibited increased mean serum triglyceride levels when compared with their gender-matched (+/+) littermates and the historical mean.

Thus, mutant mice deficient in the PRO7476 encoding gene can serve as a model for treatment of cardiovascular disease especially those diseases which are associated with dyslipidemia. PRO7476 polypeptides or its encoding gene would be useful in regulating blood lipids and in particular for maintaining normal levels of triglycerides. Thus, PRO7476 polypeptides would be useful in the treatment of such cardiovascular diseases as: hypertension, atherosclerosis, heart failure, stroke, various coronary artery diseases, and/or obesity or diabetes.

(e) Bone Metabolism & Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Bone MicroCT Analysis:

Procedure: MicroCT was also used to get very sensitive measurements of BMD. One vertebra and 1 femur were taken from a cohort of 4 wild type and 8 homozygous mice. Measurements were taken of lumbar 5 vertebra trabecular bone volume, trabecular thickness, connectivity density and midshaft femur total bone area and cortical thickness. The μCT40 scans provided detailed information on bone mass and architecture. Multiple bones were placed into sample holders and scanned automatically. Instrument software was used to select regions of interest for analysis. Trabecular bone parameters were analyzed in the fifth lumbar vertebrae (LV5) at 16 micrometer resolution and cortical bone parameters were analyzed in the femur midshaft at a resolution of 20 micrometers.

CAT-Scan Protocol:

Mice were injected with a CT contrast agent, Omnipaque 300 (Nycomed Amershan, 300 mg of iodine per ml, 0.25 ml per animal, or 2.50-3.75 g iodine/kg of body weight) intraperitoneally. After resting in the cage for ˜10 minutes, the mouse was then sedated by intraperitoneal injection of Avertin (1.25% 2,2,2,-tribromoethanol, 20 ml/kg body weight). A CAT-scan was performed using a MicroCAT scanner (ImTek, Inc.) with the anesthetized animal lying prone on the test bed. Three dimensional images were reconstructed by the Feldkamp algorithm in a cluster of workstations using an ImTek 3D RECON software.

Results:

DEXA: The male and female (−/−) mice exhibited notably increased mean bone mineral content, bone mineral content index (BMC/LBM), volumetric bone mineral density, and bone mineral density in total body, femur, and vertebrae when compared with their gender-matched (+/+) littermates and the historical means. In addition, the male and female (−/−) mice exhibited increased mean percent total body fat. Female (−/−) knockouts also showed increased total tissue mass and fat mass (g).
Micro-CT: The male (−/−) mice exhibited notably increased mean vertebral trabecular bone volume, number, thickness, and connectivity density and increased mean femoral mid-shaft cortical thickness and cross-sectional area when compared with their gender-matched (+/+) littermates and the historical means.
CAT-Scan: The (−/−) mice exhibited no obvious deformities or differences in the dimensions of the skull and facial structures. However, 2D projections revealed generally increased bone density and 3D surface views revealed thickening of the scapulas in all 3 (−/−) mice.

The (−/−) mice exhibited increased mean total body fat and increased bone mineral density measurements and trabecular bone related measurements and femoral mid-shaft cortical thickness and cross-sectional area when compared with their gender-matched (+/+) littermates. These results indicate that the knockout mutant phenotype is associated with such bone abnormalities as osteopetrosis. Osteopetrosis is a condition characterized by abnormal thickening and hardening of bone and abnormal fragility of the bones. As such, PRO7476 polypeptides or agonists thereof would be beneficial for the treatment of osteopetrosis. A phenotype associated with an increased bone mineral content, and total body and femoral bone mineral density suggests that agents which mimic these effects (e.g. antagonists of PRO1779 polypeptides) would be useful in bone healing. In addition, female mutant (−/−) mice also exhibited an increased mean percentage of body fat and increased triglyceride levels which is suggestive of an obesity phenotype. These observations suggest that mutant mice deficient in the gene which encodes PRO7476 polypeptides leads to metabolic disorders associated with accumulation of fat but also abnormal bone measurements reflective of general metabolic disorders which can be associated with obesity. Thus, PRO7476 polypeptides or agonists thereof would be useful in the treatment or prevention of such disorders as obesity or other metabolic diseases.

CAT-Scan results indicated deformities in skull and facial structures reflective of the abnormal bone phenotype for the knockout mice.

70.60. Generation and Analysis of Mice Comprising DNA111030 (UNQ3026) Gene Disruptions

In these knockout experiments, the gene encoding PRO9824 polypeptides (designated as DNA111030) (UNQ3026) was disrupted. The gene specific information for these studies is as follows: the mutated mouse gene corresponds to nucleotide reference: NM_{—}172833 Mus musculus mucosa associated lymphoid tissue lymphoma translocation gene 1 (Malti); protein reference: Q8BFT0 ACCESSION:Q8BFT0 NID: Mus musculus (Mouse). Similar to mucosa associated lymphoid tissue lymphoma translocation gene 1; the human gene sequence reference: NM_{—}006785 Homo sapiens mucosa associated lymphoid tissue lymphoma translocation gene 1 (MALT1), transcript variant 1; the human protein sequence corresponds to reference: Q9UDY8 ACCESSION:Q9UDY8 NID: Homo sapiens (Human). Mucosa associated lymphoid tissue lymphoma translocation protein 1 (EC 3.4.22.-) (MALT-lymphoma associated translocation) (Paracaspase).

The mouse gene of interest is Malti (mucosa associated lymphoid tissue lymphoma translocation gene 1), ortholog of human MALT1. Aliases include A630046N12, D430033E09Rik, paracaspase, MLT, MLT1, DKFZp434L132, caspase-like protein, MALT associated translocation, and MALT-lymphoma associated translocation.

MALT1 participates in activation of nuclear factor (NF)-kappaB by cell-surface antigen receptors on B- and T-lymphocytes. The protein consists of a death domain (Pfam accession PF00531), two immunoglobulin domains (Pfam accession PF00047), and a caspase domain (Pfam accession PF00656). The caspase domain of MALT1 contains conserved cysteine and histidine residues important for protease activity and for activation of NF-kappaB; however, MALT1 protease activity has not been detected (Uren et al, Mol Cell 6(4):961-7 (2000); Lucas et al, J Cell Sci 117(Pt 1):31-9 (2004)). MALT1 is likely to function as a ubiquitin ligase (Zhou et al, Nature 427(6970):167-71 (2004)) or as an adaptor protein that promotes ubiquitin ligase TRAF6 oligomerization and activation (Sun et al, Mol Cell 14(3):289-301 (2004)). Polyubiquitination of NF-kappaB essential modulator (NEMO), which is the regulatory subunit of the IkappaB kinase complex, results in the sequential activation of IkappaB kinase and NF-kappaB. MALT1 plays an important role in activation and proliferation of mature B- and T-lymphocytes. Translocations of the MALT1 gene are associated with formation of B-cell lymphomas of mucosa-associated lymphoid tissue (Lucas et al, J Cell Sci 117(Pt 1):31-9 (2004); Thome, Nat Rev Immunol 4(5):348-59 (2004)).

Ruefli-Brasse and coworkers; Science 302(5650):1581-4 (2003) investigated the role of MALT1 in lymphocyte activation and development using MALT1-deficient mice. They showed that T- and B-lymphocytes from MALT1 homozygous null mice displayed defective antigen receptor-induced NF-kappaB activation, cytokine production, and proliferation. They concluded that MALT1 activates IkappaB kinase complex by directly associating with it or by recruiting other proteins required for IkappaB kinase activation.

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 20 | 49 | 13 | 82 |
| Expected | 20.5 | 41 | 20.5 | 82 |

Chi-Sq.=4.59 Significance=0.100761384 (hom/n)=0.19 Avg. Litter Size=7

### Mutation Information

Mutation Type: Homologous Recombination (standard)
Description: Coding exon 1 was targeted (NCBI accession NM_{—}172833.1).
1. Wild-type Expression Panel: Expression of the target gene was detected in embryonic stem (ES) cells and in all 13 adult tissue samples tested by RT-PCR.
2. QC Expression: Disruption of the target gene was confirmed by Southern hybridization analysis.

70.60.1. Phenotypic Analysis (for Disrupted Gene: DNA111030 (UNQ3026)

(a) Overall Phenotypic Summary:

Mutation of the gene encoding the ortholog of human mucosa associated lymphoid tissue lymphoma translocation gene 1 (MALT1) resulted in generalized inflammation and immunological abnormalities in (−/−) mice. The homozygous mutant mice exhibited immunological abnormalities and generalized acute and chronic inflammation when compared with their wild-type littermates and the historical means. In addition, the male (−/−) mice exhibited increased mean volumetric and total bone mineral density as well as increased mean femoral midshaft cross-sectional area and decreased cortical thickness. Disruption of the target gene was confirmed by Southern hybridization analysis.

(b) Pathology:

Microscopic Observations: The (−/−) mice exhibited generalized acute and chronic inflammation in skeletal muscle, kidney, urinary bladder, alimentary system, and upper respiratory tract. The most severe lesion was a diffuse suppurative meningitis, occurring in a female mutant (F-147).
Gene Expression LacZ activity was not detected in the panel of tissues by immunohistochemical analysis.

(c) Immunology Phenotypic Analysis

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

T lymphocytes (T cells) are an important component of a mammalian immune response. T cells recognize antigens which are associated with a self-molecule encoded by genes within the major histocompatibility complex (MHC). The antigen may be displayed together with MHC molecules on the surface of antigen presenting cells, virus infected cells, cancer cells, grafts, etc. The T cell system eliminates these altered cells which pose a health threat to the host mammal. T cells include helper T cells and cytotoxic T cells. Helper T cells proliferate extensively following recognition of an antigen-MHC complex on an antigen presenting cell. Helper T cells also secrete a variety of cytokines, i.e., lymphokines, which play a central role in the activation of B cells, cytotoxic T cells and a variety of other cells which participate in the immune response.

In many immune responses, inflammatory cells infiltrate the site of injury or infection. The migrating cells may be neutrophilic, eosinophilic, monocytic or lymphocytic as can be determined by histologic examination of the affected tissues. Current Protocols in Immunology, ed. John E. Coligan, 1994, John Wiley & Sons, Inc.

Many immune related diseases are known and have been extensively studied. Such diseases include immune-mediated inflammatory diseases (such as rheumatoid arthritis, immune mediated renal disease, hepatobiliary diseases, inflammatory bowel disease (IBD), psoriasis, and asthma), non-immune-mediated inflammatory diseases, infectious diseases, immunodeficiency diseases, neoplasia, and graft rejection, etc. In the area of immunology, targets were identified for the treatment of inflammation and inflammatory disorders.

In the area of immunology, targets have been identified herein for the treatment of inflammation and inflammatory disorders. Immune related diseases, in one instance, could be treated by suppressing the immune response. Using neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

The following tests were performed:

Flourescence-activated Cell-sorting (FACS) Analysis

Procedure:

FACS analysis of immune cell composition from peripheral blood was performed including CD4, CD8 and T cell receptor to evaluate T lymphocytes, CD19 for B lymphocytes, CD45 as a leukocyte marker and pan NK for natural killer cells. The FACS analysis was carried out on 2 wild type and 6 homozygous mice and included cells derived from thymus, spleen, bone marrow and lymph node.

In these studies, analyzed cells were isolated from thymus, peripheral blood, spleen, bone marrow and lymph nodes. Flow cytometry was designed to determine the relative proportions of CD4 and CD8 positive T cells, B cells, NK cells and monocytes in the mononuclear cell population. A Becton-Dickinson FACSCalibur 3-laser FACS machine was used to assess immune status. For Phenotypic Assays and Screening, this machine records CD4+/CD8−, CD8+/CD4−, NK, B cell and monocyte numbers in addition to the CD4+/CD8+ ratio. The mononuclear cell profile was derived by staining a single sample of lysed peripheral blood from each mouse with a panel of six lineage-specific antibodies: CD45 PerCP, anti-TCRb APC, CD4 PE, CD8 FITC, pan-NK PE, and CD19 FITC. The two FITC and PE labeled antibodies stain mutually exclusive cell types. The samples were analyzed using a Becton Dickinson FACSCalibur flow cytometer with CellQuest software.

Results:

The (−/−) mice exhibited decreased percentages of CD4/CD8 DP cells and increased percentages of TCRB+ cells in the thymus when compared with the (+/+) mice. The (−/−) mice also showed decreased CD21hi/CD23med B cells in the spleen compared to the (+/+) mice. Thus, (−/−) mice exhibit a decreased T cell levels in the thymus and decreased B cell progenitors in the spleen.

Acute Phase Response:

Test Description: Bacterial lipopolysaccharide (LPS) is an endotoxin, and as such is a potent inducer of an acute phase response and systemic inflammation. The Level I LPS mice were injected intraperitoneally (i.p.) with a sublethal dose of LPS in 200 μL sterile saline using a 26 gauge needle. The doses were based on the average weight of the mice tested at 1 μg/g body weight 3 hours after injection; a 100 ul blood sample was then taken and analyzed for the presence of TNFa, MCP-1, and IL-6 on the FACSCalibur instrument.

Results:

The (−/−) mice exhibited increased mean serum TNF-alpha and IL-6 responses to LPS challenge when compared with their (+/+) littermates and the historical means.

In summary, the LPS endotoxin challenge demonstrated that knockout mice deficient in the gene encoding PRO9824 polypeptides exhibit immunological abnormalities when compared with their wild-type littermates. In particular, the mutant mice exhibited an increased ability to elicit an immunological response (TNF-alpha and IL-6 production) when challenged with the LPS endotoxin indicating a proinflammatory response. TNF-alpha and IL-6 contribute to the later stages of B cell activation. TNF-alpha is an important inflammatory mediator. In addition, both TNF-alpha and IL-6 play a critical role in inducing the acute phase response and systemic inflammation. TNF-alpha can substitute for the membrane-bound signal in macrophage activation (thus serving as an effector molecule).

Ovalbumin Challenge

Procedure: This assay was carried out on 7 wild type and 8 homozygous mice. Chicken ovalbumin (OVA) is a T-cell dependent antigen, which is commonly used as a model protein for studying antigen-specific immune responses in mice. OVA is non-toxic and inert and therefore will not cause harm to the animals even if no immune response is induced. The murine immune response to OVA has been well characterized, to the extent that the immunodominant peptides for eliciting T cell responses have been identified. Anti-OVA antibodies are detectable 8 to 10 days after immunization using enzyme-linked immunosorbent assay (ELIZA), and determination of different isotypes of antibodies gives further information on the complex processes that may lead to a deficient response in genetically engineered mice.

As noted above, this protocol assesses the ability of mice to raise an antigen-specific immune response. Animals were injected IP with 50 mg of chicken ovalbumin emulsified in Complete Feund's Adjuvant and 14 days later the serum titer of anti-ovalbumin antibodies (IgM, IgG1 and IgG2 subclasses) was measured. The amount of OVA-specific antibody in the serum sample is proportional to the Optical Density (OD) value generated by an instrument that scans a 96-well sample plate. Data was collected for a set of serial dilutions of each serum sample.

Results of this Challenge:

The (−/−) mice exhibited decreased mean serum IgG1 and IgG2a responses to ovalbumin challenge when compared with their (+/+) littermates and the historical mean.

In summary, the ovalbumin challenge studies indicate that knockout mice deficient in the gene encoding PRO9824 polypeptides exhibit immunological abnormalities when compared with their wild-type littermates. In particular, the mutant mice exhibited a decreased ability to elicit an immunological response when challenged with the T-cell dependent OVA antigen. Thus, PRO9824 polypeptides or agonists thereof, would be useful for stimulating the immune system (such as T cell proliferation) and would find utility in the cases wherein this effect would be beneficial to the individual such as in the case of leukemia, and other types of cancer, and in immunocompromised patients, such as AIDS sufferers. Accordingly, inhibitors (antagonists) of PRO9824 polypeptides would be useful for inhibiting the immune response and thus would be useful candidates for suppressing harmful immune responses, e.g. in the case of graft rejection or graft-versus-host diseases.

Hematology Analysis:

Test Description: Blood tests are carried out by Abbott's Cell-Dyn 3500R, an automated hematology analyzer. Some of its features include a five-part WBC differential. ‘Patient’ reports can cover over 22 parameters in all.

Results:

Hematology: The (−/−) mice exhibited an increased mean total white blood cell count when compared with their (+/+) littermates and the historical mean, characterized by increased absolute neutrophil, lymphocyte, monocyte, and basophil counts.

In summary, the hematology results indicate that the homozygous mutant mice exhibited an increased white blood cell count compared to their (+/+) littermate controls indicating elevated levels of precursors of macrophages. These results indicate that the homozygous (−/−) knockout mice exhibit an abnormal immunological phenotype.

Serum Immunoglobulin Isotyping Assay:

The Serum Immunoglobulin Isotyping Assay is performed using a Cytometric Bead Array (CBA) kit. This assay is used to rapidly identify the heavy and light chain isotypes of a mouse monoclonal antibody in a single sample. The values expressed are “relative fluorescence units” and are based on the detection of kappa light chains. Any value <6 is not significant.

Results:

Serum Immunoglobulins: The (−/−) mice exhibited decreased mean serum IgM, IgA, IgG3, IgG2b and IgG2a levels when compared with that of their (+/+) littermates, the (+/+) mice within the project run, and the historical median.

Mutant (−/−) mice exhibited decreased levels of IgM, IgA, and IgG3 serum immunoglobulins compared to their gender-matched (+/+) littermates. IgM immunoglobulins are the first to be produced in a humoral immune response for neutralization of bacterial toxins and are particularly important in activating the complement system. Likewise, IgG immunoglobulins have neutralization effects and to a lesser extent are important for activation of the complement system. IgA mainly functions as an epithelial cell protector which can neutralize bacterial toxins and viruses. Although no obvious disease susceptibility is associated with selective IgA defects, they are commoner in people with chronic lung disease than in the general population. This suggests that lack of IgA may result in a predisposition to lung infections with various pathogens and is consistent with the role of IgA in defense at the body surfaces. In this case, the phenotype observed for knockout mice resulted in an increase in IgA serum levels suggesting that PRO9824 polypeptides or agonists thereof would be useful for a natural immunity protection against skin infections and more importantly may prevent susceptibility to lung infections. IgG3, IgG2a and IgG2b immunoglobulins have neutralization effects and to a lesser extent are important for activation of the complement system. The observed phenotype suggests that the PRO9824 polypeptide is a regulator of inflammatory responses. These immunological abnormalities suggest that PRO9824 polypeptides or agonists thereof would be useful agents which could stimulate the immune system (such as T cell proliferation) and would find utility in the cases wherein this effect would be beneficial to the individual such as in the case of leukemia, and other types of cancer, and in immunocompromised patients, such as AIDS sufferers. Accordingly, PRO9824 polypeptide antagonists or inhibitors would be useful in inhibiting the immune response and would be useful candidates for suppressing harmful immune responses, e.g. in the case of graft rejection or graft-versus-host diseases.

(d) Bone Metabolism & Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Bone MicroCT Analysis:

Procedure: MicroCT was also used to get very sensitive measurements of BMD. One vertebra and 1 femur were taken from a cohort of 4 wild type and 8 homozygous mice. Measurements were taken of lumbar 5 vertebra trabecular bone volume, trabecular thickness, connectivity density and midshaft femur total bone area and cortical thickness. The μCT40 scans provided detailed information on bone mass and architecture. Multiple bones were placed into sample holders and scanned automatically. Instrument software was used to select regions of interest for analysis. Trabecular bone parameters were analyzed in the fifth lumbar vertebrae (LV5) at 16 micrometer resolution and cortical bone parameters were analyzed in the femur midshaft at a resolution of 20 micrometers.

Results:

DEXA: The male (−/−) mice exhibited increased mean volumetric and total body bone mineral density when compared with their gender-matched (+/+) littermates and the historical means.
Micro-CT: The male (−/−) mice exhibited increased mean femoral midshaft cross-sectional area when compared with their gender-matched (+/+) littermates and the historical mean.

In summary, the (−/−) mice exhibited increased volumetric and bone mineral density and femoral mid-shaft cross-sectional area when compared with their gender-matched (+/+) littermates. These results indicate that the knockout mutant phenotype is associated with such bone abnormalities as osteopetrosis. Osteopetrosis is a condition characterized by abnormal thickening and hardening of bone and abnormal fragility of the bones. As such, PRO9824 polypeptides or agonists thereof would be beneficial for the treatment of osteopetrosis or other osteo-related diseases. On the other hand, inhibitors or antagonists of PRO9824 polypeptides would be useful in bone healing.

70.61. Generation and Analysis of Mice Comprising DNA148004-2882 (UNQ5923) Gene Disruptions

In these knockout experiments, the gene encoding PRO19814 polypeptides (designated as DNA148004-2882) (UNQ5923) was disrupted. The gene specific information for these studies is as follows: the mutated mouse gene corresponds to nucleotide reference: NM_{—}026324 Mus musculus kin of IRRE like 3 (Drosophila) (Kirrel3); protein reference:Q810H3 ACCESSION:Q810H₃NID: Mus musculus (Mouse). Membrane protein mKirre; the human gene sequence reference:NM_{—}032531 Homo sapiens kin of IRRE like 3 (Drosophila) (KIRREL3); the human protein sequence corresponds to reference: Q81ZU9 ACCESSION:Q81ZU9 NID: Homo sapiens (Human). NPEH2.

The mouse gene of interest is Kirrel3 (kin of IRRE like 3 [Drosophila]), ortholog of human KIRREL3. Aliases include SST4, NEPH2, mKirre, mKIAA 1867, 1500010O20Rik, membrane protein mKirre, X kin of IRRE like 3 (Drosophila), KIRRE, and KIAA1867.

KIRREL3 is a type I plasma membrane protein that likely functions as a cell adhesion molecule or signaling molecule. The protein consists of a signal peptide, five immunoglobulin-like domains, a transmembrane segment, and a cytoplasmic C-terminal domain. The cytoplasmic domain of all 3 KIRREL family members contains a Grb2 SH2 binding site and a PDZK1 binding site, suggesting that KIRREL3 functions as a receptor (Sellin et al, FASEB J 17(1):115-7 (2003)). The extracellular domain of KIRREL3 on bone marrow stromal cells may be shed by matrix metalloproteinases to possibly function as a ligand that inhibits hematopoietic stem cell differentiation or as a homing receptor that controls hematopoietic stem cell migration (Ueno et al, Nat Immunol 4(5):457-63 (2003)).

KIRREL3 is expressed in a number of different tissues and cell types, including brain, heart, nervous system, kidney, renal glomeruli, a renal glomerular podocyte cell line, bone marrow stromal cells, and a lung carcinoma cell line. In bone and other tissues involved in hematopoiesis, KIRREL3 is likely to play a role in hematopoietic stem cell support. In kidney, KIRREL3 may play a role in glomerular filtration (Sellin et al, FASEB J 17(1):115-7 (2003); Ueno et al, Nat Immunol 4(5):457-63 (2003)).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 22 | 33 | 19 | 74 |
| Expected | 18.5 | 37 | 18.5 | 74 |

Chi-Sq.=0.57 Significance=0.7520143 (hom/n)=0.23 Avg. Litter Size=8

### Mutation Information

Mutation Type: Homologous Recombination (standard)
Description: Coding exon 5 was targeted (NCBI accession NM_{—}026324.1).
1. Wild-type Expression Panel: Expression of the target gene was detected in embryonic stem (ES) cells and in all 13 adult tissue samples tested by RT-PCR, except liver.
2. QC Expression: Disruption of the target gene was confirmed by Southern hybridization analysis.

70.61.1. Phenotypic Analysis (for Disrupted Gene: DNA148004-2882 (UNQ5923)

(a) Overall Phenotypic Summary:

Mutation of the gene encoding the ortholog of human kin of IRRE like 3 (Drosophila) (KIRREL3) resulted in decreased serum insulin in male (−/−) mice. Both male and female knockout mice showed increased total tissue mass, lean body mass, total body fat content as well as increased blood triglyceride levels. The male (−/−) mice exhibited decreased mean vertebral bone mineral density and decreased mean vertebral trabecular bone volume, number, thickness and connectivity density. Disruption of the target gene was confirmed by Southern hybridization analysis.

(b) Phenotypic Analysis: Cardiology

In the area of cardiovascular biology, targets were identified herein for the treatment of hypertension, atherosclerosis, heart failure, stroke, various coronary artery diseases, dyslipidemias such as high cholesterol (hypercholesterolemia) and elevated serum triglycerides (hypertriglyceridemia), diabetes and/or obesity. The phenotypic tests included the measurement of serum cholesterol and triglycerides.

Blood Lipids

Procedure: A cohort of 4 wild type, 4 heterozygous and 8 homozygous mice were tested in this assay. High cholesterol levels and increased triglyceride blood levels are recognized risk factors in the development of cardiovascular disease and/or diabetes. Measuring blood lipids facilitates the finding of biological switches that regulate blood lipid levels Inhibition of factors which elevate blood lipid levels may be useful for reducing the risk for cardiovascular disease. In these blood chemistry tests, measurements were recorded using the COBAS Integra 400 (mfr: Roche).

Results:

The male (−/−) mice exhibited notably increased mean serum triglyceride and cholesterol levels when compared with their gender-matched (+/+) littermates and the historical mean.

Thus, mutant mice deficient in the PRO19814 encoding gene can serve as a model for treatment of cardiovascular disease especially those diseases which are associated with dyslipidemia. PRO19814 polypeptides or its encoding gene would be useful in regulating blood lipids and in particular for maintaining normal levels of triglycerides and cholesterol. Thus, PRO19814 polypeptides would be useful in the treatment of such cardiovascular diseases as: hypertension, atherosclerosis, heart failure, stroke, various coronary artery diseases, dyslipidemia, hypercholestremia, hypertriglyceridemia, and/or obesity or diabetes.

(c) Blood Chemistry

Blood chemistry analysis was performed using the COBAS Integra 400 (mfr: Roche) in its clinical settings for running blood chemistry tests on mice.

Insulin Data:

Test Description: Lexicon Genetics uses the Cobra II Series Auto-Gamma Counting System in its clinical settings for running quantitative Insulin assays on mice.

Results:

The (−/−) mice exhibited a decreased mean serum insulin level when compared with their gender-matched (+/+) littermates and the historical mean.

Mutant (−/−) mice deficient in the gene encoding PRO19814 polypeptides show a phenotype consistent with dyslipidemia. Insulin levels were decreased which can be indicative of diabetes. Thus, antagonists or inhibitors of PRO19814 polypeptides or its encoding gene would mimic these metabolic effects. On the other hand, PRO19814 polypeptides or agonists thereof would be useful in the prevention and/or treatment of such metabolic disorders as diabetes.

(d) Bone Metabolism & Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Bone MicroCT Analysis:

Procedure: MicroCT was also used to get very sensitive measurements of BMD. One vertebra and 1 femur were taken from a cohort of 4 wild type and 8 homozygous mice. Measurements were taken of lumbar 5 vertebra trabecular bone volume, trabecular thickness, connectivity density and midshaft femur total bone area and cortical thickness. The μCT40 scans provided detailed information on bone mass and architecture. Multiple bones were placed into sample holders and scanned automatically. Instrument software was used to select regions of interest for analysis. Trabecular bone parameters were analyzed in the fifth lumbar vertebrae (LV5) at 16 micrometer resolution and cortical bone parameters were analyzed in the femur midshaft at a resolution of 20 micrometers.

Results:

DEXA: Both the male and female (−/−) mice exhibited increased total tissue mass, lean body mass, total body fat content as well as decreased mean vertebral bone mineral density when compared with their gender-matched (+/+) littermates and the historical mean.
Micro-CT: The male (−/−) mice exhibited decreased mean vertebral trabecular bone volume, number, thickness, and connectivity density when compared with their gender-matched (+/+) littermates and the historical means.

The (−/−) mice analyzed by DEXA and bone micro CT analysis exhibited decreased bone measurements when compared with their (+/+) littermates, suggestive of abnormal bone disorders. However, the mutant (−/−) mice also exhibited increased body mass measurements and an increased mean percentage of body fat suggestive of an obesity phenotype especially in view of blood chemistry analysis showing elevated serum levels of both cholesterol and triglycerides. These observations suggest that mutant mice deficient in the gene which encodes PRO19814 polypeptides leads to metabolic disorders associated with accumulation of fat but also abnormal bone measurements reflective of general metabolic disorders which can be associated with obesity. Thus, PRO19814 polypeptides or agonists thereof would be useful in the treatment or prevention of such disorders as obesity or other metabolic diseases. However, the negative bone phenotype would also suggest that PRO19814 polypeptides or agonists thereof would be useful for maintaining bone homeostasis. In addition, PRO19814 polypeptides would be useful in bone healing or for the treatment of arthritis or osteoporosis, whereas antagonists (or inhibitors) of PRO19814 polypeptides or its encoding gene would lead to abnormal or pathological bone disorders including inflammatory diseases associated with abnormal bone metabolism including arthritis, osteoporosis and osteopenia.

70.62. Generation and Analysis of Mice Comprising DNA144839 (UNQ5930) Gene Disruptions

In these knockout experiments, the gene encoding PRO19836 polypeptides (designated as DNA144839) (UNQ5930) was disrupted. The gene specific information for these studies is as follows: the mutated mouse gene corresponds to nucleotide reference: NM_{—}018776 ACCESSION:NM_{—}018776 NID: gi 9055199 refNM_{—}018776.1 Mus musculus cytokine receptor-like factor 3 (Crlf3); protein reference: Q9Z2L7 ACCESSION:Q9Z2L7 NID: Mus musculus (Mouse). CYTOKINE RECEPTOR RELATED PROTEIN 4; the human gene sequence reference: NM_{—}015986 Homo sapiens cytokine receptor-like factor 3 (CRLF3); the human protein sequence corresponds to reference: Q9Y6M8 ACCESSION:Q9Y6M8 NID: Homo sapiens (Human). Cytokine receptor related protein 4.

The mouse gene of interest is Crlf3 (cytokine receptor-like factor 3), ortholog of human CRLF3. Aliases include Crlf2, Creme9, Cytor4, MGC20661, cytokine receptor-like factor 2, cytokine receptor-like molecule 9, and cytokine receptor related protein 4.

CRLF3 is a hypothetical non-secretory protein that contains a single fibronectin type 3 domain (SMART accession SM00060). The function of CRLF3 is not known.

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 19 | 26 | 10 | 55 |
| Expected | 13.75 | 27.5 | 13.75 | 55 |

Chi-Sq.=0.57 Significance=0.7520143 (hom/n)=0.25 Avg. Litter Size=6

### Mutation Information

Mutation Type: Homologous Recombination (standard)
Description: Coding exons 3 through 6 were targeted (NCBI accession NM_{—}018776.1).
1. Wild-type Expression Panel: Expression of the target gene was detected in all 13 adult tissue samples tested by RT-PCR, except kidney and adipose.
2. QC Expression: Disruption of the target gene was confirmed by Southern hybridization analysis.

70.62.1. Phenotypic Analysis (for Disrupted Gene: DNA144839 (UNQ5930)

(a) Overall Phenotypic Summary:

Mutation of the gene encoding the ortholog of human cytokine receptor-like factor 3 (CRLF3) resulted in decreased platelets in (−/−) mice as well as increased mean serum IgM and IgG2a levels. Disruption of the target gene was confirmed by Southern hybridization analysis.

(b) Immunology Phenotypic Analysis

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

T lymphocytes (T cells) are an important component of a mammalian immune response. T cells recognize antigens which are associated with a self-molecule encoded by genes within the major histocompatibility complex (MHC). The antigen may be displayed together with MHC molecules on the surface of antigen presenting cells, virus infected cells, cancer cells, grafts, etc. The T cell system eliminates these altered cells which pose a health threat to the host mammal. T cells include helper T cells and cytotoxic T cells. Helper T cells proliferate extensively following recognition of an antigen-MHC complex on an antigen presenting cell. Helper T cells also secrete a variety of cytokines, i.e., lymphokines, which play a central role in the activation of B cells, cytotoxic T cells and a variety of other cells which participate in the immune response.

In many immune responses, inflammatory cells infiltrate the site of injury or infection. The migrating cells may be neutrophilic, eosinophilic, monocytic or lymphocytic as can be determined by histologic examination of the affected tissues. Current Protocols in Immunology, ed. John E. Coligan, 1994, John Wiley & Sons, Inc.

Many immune related diseases are known and have been extensively studied. Such diseases include immune-mediated inflammatory diseases (such as rheumatoid arthritis, immune mediated renal disease, hepatobiliary diseases, inflammatory bowel disease (IBD), psoriasis, and asthma), non-immune-mediated inflammatory diseases, infectious diseases, immunodeficiency diseases, neoplasia, and graft rejection, etc. In the area of immunology, targets were identified for the treatment of inflammation and inflammatory disorders.

In the area of immunology, targets have been identified herein for the treatment of inflammation and inflammatory disorders. Immune related diseases, in one instance, could be treated by suppressing the immune response. Using neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

The following tests were performed:

Hematology Analysis:

Test Description: Blood tests are carried out by Abbott's Cell-Dyn 3500R, an automated hematology analyzer. Some of its features include a five-part WBC differential. ‘Patient’ reports can cover over 22 parameters in all.

Results:

Hematology: The (−/−) mice exhibited a decreased mean platelet count (17.9% decrease) when compared with their (+/+) littermates and the historical mean.

Thus, mutant mice deficient in the DNA144839 gene resulted in a phenotype related to coagulation disorders. In this regard, PRO19836 polypeptides or agonists thereof would be useful in treating disorders related to abnormal blood coagulation such as hemophilia.

Serum Immunoglobulin Isotyping Assay:

The Serum Immunoglobulin Isotyping Assay is performed using a Cytometric Bead Array (CBA) kit. This assay is used to rapidly identify the heavy and light chain isotypes of a mouse monoclonal antibody in a single sample. The values expressed are “relative fluorescence units” and are based on the detection of kappa light chains. Any value <6 is not significant.

Results:

Serum Immunoglobulins: The (−/−) mice exhibited increased mean serum IgM and IgG2a levels when compared with that of their (+/+) littermates, the (+/+) mice within the project run, and the historical median.

Mutant (−/−) mice exhibited elevation of IgM and IgG2a serum immunoglobulins compared to their gender-matched (+/+) littermates. IgM immunoglobulins are the first to be produced in a humoral immune response for neutralization of bacterial toxins and are particularly important in activating the complement system. Likewise, IgG2a immunoglobulins have neutralization effects and to a lesser extent are important for activation of the complement system. The observed phenotype suggests that the PRO19836 polypeptide is a negative regulator of inflammatory responses. These immunological abnormalities suggest that inhibitors (antagonists) of PRO19836 polypeptides may be important agents which could stimulate the immune system (such as T cell proliferation) and would find utility in the cases wherein this effect would be beneficial to the individual such as in the case of leukemia, and other types of cancer, and in immunocompromised patients, such as AIDS sufferers. Accordingly, PRO19836 polypeptides or agonists thereof would be useful in inhibiting the immune response and would be useful candidates for suppressing harmful immune responses, e.g. in the case of graft rejection or graft-versus-host diseases.

70.63. Generation and Analysis of Mice Comprising DNA150157-2898 (UNQ6077) Gene Disruptions

In these knockout experiments, the gene encoding PRO20088 polypeptides (designated as DNA150157-2898) (UNQ6077) was disrupted. The gene specific information for these studies is as follows: the mutated mouse gene corresponds to nucleotide reference: NM_{—}177389 Mus musculus melanoma inhibitory activity 3 (Mia3); protein reference: Q8BI84 ACCESSION: Q8BI84 NID: Mus musculus (Mouse). Mus musculus adult retina cDNA, RIKEN full-length enriched library, clone:A930039G15 product: weakly similar to NPIP-LIKE PROTEIN; the human gene sequence reference: XM_{—}496436 PREDICTED: Homo sapiens similar to RIKEN cDNA A930039G15 gene (LOC440718); the human protein sequence corresponds to reference: XP_{—}496436 PREDICTED: similar to RIKEN cDNA A930039G15 gene [Homo sapiens].

The mouse gene of interest is Mia3 (melanoma inhibitory activity 3), ortholog of human UNQ6077. Aliases include Tango, A930039G15Rik, and AAAP6077.

Human UNQ6077 and mouse ortholog Mia are genes likely to encode large proteins; however, current predictions for these genes remain ambiguous. Human UNQ6077 appears to encode a protein of about 2000 amino acids. The hypothetical protein contains a signal peptide, a src homology 3 domain, and a potential C-terminal conserved domain of about 700 amino acids. This C-terminal region displays weak similarity with conserved domains mitotic checkpoint protein (Pfam accession PF05557; E-value=0.04), vicilin N-terminal region (Pfam accession PF04702; E-value=0.06), TolA protein (Pfam accession PF06519; E-value=0.07), and myosin tail (Pfam accession PF01576; E-value=0.01). Mouse Mia3 encodes a hypothetical 1239-amino acid protein, consisting of a signal peptide, a src homology 3 domain, an ambiguous conserved domain, and a C-terminal transmembrane segment. The ambiguous conserved domain is similar to the N-terminal part of trypsin-alpha amylase inhibitor domain (SMART accession SM00499) and menin domain (PFAM accession PF05053). The predicted cell location of both human and mouse hypothetical proteins are ambiguous.

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 16 | 36 | 0 | 52 |
| Expected | 13 | 26 | 13 | 52 |

Chi-Sq.=21.46 Significance=2.1878644E-5 (hom/n)=0.06 Avg. Litter Size=5

### Mutation Information

Mutation Type: Homologous Recombination (standard)
Description: Coding exons 2 and 3 were targeted (NCBI accession NM_{—}177389.2). UNQ6077 deletion starts 11 nucleotides 3′ of exon 2 and stops 10 nucleotides into intron between exons 3 and 4. Fourth exon is still transcribed in UNQ6077 (−/−). Analysis of the human TANGO gene and RT-PCR indicate that there are likely to be alternative 3′ transcripts.
1. Wild-type Expression Panel: Expression of the target gene was detected in embryonic stem (ES) cells and in all 13 adult tissue samples tested by RT-PCR, except skeletal muscle and bone.
2. QC Expression: Disruption of the target gene was confirmed by Southern hybridization analysis.

70.63.1. Phenotypic Analysis (for Disrupted Gene: DNA150157-2898 (UNQ6077)

(a) Overall Phenotypic Summary:

Mutation of the gene encoding the ortholog of human UNQ6077 resulted in lethality of (−/−) mutants. UNQ6077 homozygous knockout (−/−) mice showed defective bone formation during embryonic development (E18.5) compared to the wildtype (+/+) mice. Gene disruption was confirmed by Southern blot.

Discussion Related to Embryonic Developmental Abnormality of Lethality:

Embryonic lethality in knockout mice usually results from various serious developmental problems including but not limited to neurodegenerative diseases, angiogenic disorders, inflammatory diseases, or where the gene/protein has an important role in basic cell signaling processes in many cell types. In addition, embryonic lethals are useful as potential cancer models. Likewise, the corresponding heterozygous (+/−) mutant animals are particularly useful when they exhibit a phenotype and/or a pathology report which reveals highly informative clues as to the function of the knocked-out gene. For instance, EPO knockout animals were embryonic lethals, but the pathology reports on the embryos showed a profound lack of RBCs.

(b) Pathology

Microscopic Observations: There were 45 embryos observed at day 12.5:5 (−/−) embryos, 14 (+/−) embryos, 10 (+/+) embryos, and 16 resorption moles. Of the 16 resorption moles, 7 were from a single female.

Gene Expression: LacZ activity was not detected in the panel of tissues by immunohisto chemical analysis.

(c) Further Analysis of UNQ6077 Embryonic Lethality

Intercrosses between UNQ6077 heterozygous mice were set up as timed matings to produce homozygous mutant pups. Pups were delivered by cesarian on embryonic day 18.5. Skeletal preparations were produced as per Solloway et al., Dev Genet. 22: 321-339 (1998). Briefly, E18.5 embryos were euthanized on abed of dry ice, eviscerated and skinned. After being dehydrated in 100% ethanol followed by acetone overnight, embryos were stained for two days in a solution containing 0.06% Alcian Blue, 0.02% Alizarin Red, 5% glacial acetic acid, and 60% ethanol. Stained embryos were subsequently cleared in two changes of 1% potassium hydroxide over several days, transferred through a potassium hydroxide/glycerol series and were finally placed into 80% glycerol for storage and photography.

### Results:

UNQ6077 homozygous knockout (−/−) mice showed defective bone formation during embryonic development (E18.5) compared to the wildtype (+/+) mice. The skeletal preparations (described above) showed extensive defective bone formation in the (−/−) embryonic pups as illustrated by the bone-specific staining material in the E18.5 (−/−) pups. Three distinct types of bone formation were shown to be defective in their development. Specifically, dermal bone was found to be completely absent, perichondral bone was either completely absent or severely affected and endochondral bone was severely affected when compared to the same three types of bone in the wildtype (+/+) pups (E18.5).

These studies illustrate that UNQ6077encoding PRO20088 polypeptides is essential for normal bone development. PRO20088 polypeptides or agonists thereof are essential for promoting normal bone development during embryonic development. Bone modulation is a dynamic process and it is likely that PRO20088 polypeptides would be important in maintaining normal bone metabolism and would be useful in the treatment of such bone disorders as osteoporosis. Antagonists (inhibitors) of UNQ6077 would mimic the negative bone phenotype observed in the knockout (−/−) pups.

70.64. Generation and Analysis of Mice Comprising DNA295801 (UN09659) Gene Disruptions

In these knockout experiments, the gene encoding PRO70789 polypeptides (designated as DNA295801) (UNQ9659) was disrupted. The gene specific information for these studies is as follows: the mutated mouse gene corresponds to nucleotide reference: AK033906 ACCESSION:AK033906 NID: gi 26083649 dbj AK033906.1 Mus musculus adult male diencephalon cDNA, RIKEN full-length enriched library, clone:9330112P04 product:hypothetical protein, full insert sequence; protein reference: Q9CTN8 ACCESSION:Q9CTN8 NID: Mus musculus (Mouse). A930031L14Rik protein (Fragment); the human gene sequence reference: NM_{—}199000 Homo sapiens lipoma HMGIC fusion partner-like 3 (LHFPL3); the human protein sequence corresponds to reference: Q86UP9 ACCESSION:Q86UP9 NID: Homo sapiens (Human). Lipoma HMGIC fusion-partner-like protein.

The mouse gene of interest is RIKEN cDNA A930031L14 gene, ortholog of human LHFPL3 (lipoma HMGIC fusion partner-like 3).

LHFPL3 is a likely plasma membrane protein, containing a signal peptide and three transmembrane segments. LHFPL3 belongs to a family of genes that includes lipoma HMGIC fusion partner (LHFP), a fusion partner of HMGIC gene in human lipomas (Petit et al, Genomics 57(3):438-41 (1999)). The function of LHFPL3 is not known.

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 13 | 31 | 22 | 66 |
| Expected | 16.5 | 33 | 16.5 | 66 |

Chi-Sq.=0.41 Significance=0.8146473 (hom/n)=0.25 Avg. Litter Size=0

### Mutation Information

Mutation Type: Homologous Recombination (standard)
Description: Coding exon 1 was targeted (NCBI accession AK020916.1).
1. Wild-type Expression Panel: Expression of the target gene was detected in brain, spinal cord, eye, and kidney among the 13 adult tissue samples tested by RT-PCR.
2. QC Expression: Disruption of the target gene was confirmed by Southern hybridization analysis.

70.64.1. Phenotypic Analysis (for Disrupted Gene: DNA295801 (UNQ9659)

(a) Overall Phenotypic Summary:

Mutation of the gene encoding the ortholog of human lipoma HMGIC fusion partner-like 3 (LHFPL3) resulted in a decreased mean skin fibroblast proliferate rate in (−/−) mice. The mutant (−/−) mice also showed augmentation of circadian rhythm. The female (−/−) mice exhibited increased mean total body bone mineral density. Gene disruption was confirmed by Southern blot.

(b) Adult Skin Cell Proliferation:

Procedure: Skin cells were isolated from 16 week old animals (2 wild type and 4 homozygous mice). These were developed into primary fibroblast cultures and the fibroblast proliferation rates were measured in a strictly controlled protocol. The ability of this assay to detect hyper-proliferative and hypo-proliferative phenotypes has been demonstrated with p53 and Ku80. Proliferation was measured using Brdu incorporation.

Specifically, in these studies the skin fibroblast proliferation assay was used. An increase in the number of cells in a standardized culture was used as a measure of relative proliferative capacity. Primary fibroblasts were established from skin biopsies taken from wild type and mutant mice. Duplicate or triplicate cultures of 0.05 million cells were plated and allowed to grow for six days. At the end of the culture period, the number of cells present in the culture was determined using a electronic particle counter.

Results:

The female (−/−) mice exhibited a decreased mean skin fibroblast proliferation rate when compared with their gender-matched (+/+) littermates.

Thus, homozygous mutant mice demonstrated a hypo-proliferative phenotype. As suggested by these observations, antagonists or inhibitors of PRO70789 polypeptides would mimic this hypo-proliferative phenotype and could function as tumor suppressors and would be useful in decreasing abnormal cell proliferation.

(c) Phenotypic Analysis: CNS/Neurology

In the area of neurology, analysis focused herein on identifying in vivo validated targets for the treatment of neurological and psychiatric disorders including depression, generalized anxiety disorders, attention deficit hyperactivity disorder, obsessive compulsive disorder, schizophrenia, cognitive disorders, hyperalgesia and sensory disorders. Neurological disorders include the category defined as “anxiety disorders” which include but are not limited to: mild to moderate anxiety, anxiety disorder due to a general medical condition, anxiety disorder not otherwise specified, generalized anxiety disorder, panic attack, panic disorder with agoraphobia, panic disorder without agoraphobia, posttraumatic stress disorder, social phobia, specific phobia, substance-induced anxiety disorder, acute alcohol withdrawal, obsessive compulsive disorder, agoraphobia, bipolar disorder I or II, bipolar disorder not otherwise specified, cyclothymic disorder, depressive disorder, major depressive disorder, mood disorder, substance-induced mood disorder. In addition, anxiety disorders may apply to personality disorders including but not limited to the following types: paranoid, antisocial, avoidant behavior, borderline personality disorders, dependent, histronic, narcissistic, obsessive-compulsive, schizoid, and schizotypal.

Procedure:

Behavioral screens were performed on a cohort of 4 wild type, 4 heterozygous and 8 homozygous mice. All behavioral tests were done between 12 and 16 weeks of age unless reduced viability necessitates earlier testing.

Circadian Test Description:

Female mice are individually housed at 4 pm on the first day of testing in 48.2 cm×26.5 cm home cages and administered food and water ad libitum. Animals are exposed to a 12-hour light/dark cycle with lights turning on at 7 am and turning off at 7 pm. The system software records the number of beam interruptions caused by the animal's movements, with beam breaks automatically divided into ambulations. Activity is recorded in 60, one-hour intervals during the three-day test. Data generated are displayed by median activity levels recorded for each hour (circadian rhythm) and median total activity during each light/dark cycle (locomotor activity) over the three-day testing period.

Results:

The (−/−) mice exhibited an augmentation of circadian rhythm (increased ambulatory counts) during the 12-hour habituation period of home-cage activity testing when compared with their gender-matched (+/+) littermates. These findings demonstrate a hyperactive or increased anxiety phenotype in the mutant (−/−) mice. Antagonists or inhibitors of PRO70789 polypeptides would be expected to mimic this neurological phenotype. Whereas PRO70789 polypeptides or agonists thereof would be useful in the treatment of such neurological disorders as mild to moderate anxiety, generalized anxiety disorder, posttraumatic stress disorder, obsessive compulsive disorder or bipolar disorder.

(d) Bone Metabolism & Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Results:

DEXA: The female (−/−) mice exhibited increased mean total body bone mineral density when compared with their gender-matched (+/+) littermates and the historical mean.

In summary, the (−/−) mice exhibited increased total body bone mineral density when compared with their gender-matched (+/+) littermates. These results indicate that the knockout mutant phenotype is associated with such bone abnormalities as osteopetrosis. Osteopetrosis is a condition characterized by abnormal thickening and hardening of bone and abnormal fragility of the bones. As such, PRO70789 polypeptides or agonists thereof would be beneficial for the treatment of osteopetrosis or other osteo-related diseases. On the other hand, inhibitors or antagonists of PRO70789 polypeptides would be useful in bone healing.

70.65. Generation and Analysis of Mice Comprising DNA255219 (UNQ11632) Gene Disruptions

In these knockout experiments, the gene encoding PRO50298 polypeptides (designated as DNA255219) (UNQ11632) was disrupted. The gene specific information for these studies is as follows: the mutated mouse gene corresponds to nucleotide reference: NM_{—}145580 ACCESSION:NM_{—}145580 NID: gi 21704165 ref NM_{—}145580.1 Mus musculus expressed sequence AW553050 (AW553050); protein reference: Q8VE58 ACCESSION:Q8VE58 NID: Mus musculus (Mouse). Similar to hypothetical protein FLJ22573; the human gene sequence reference: NMO24660 ACCESSION:NMO24660 NID: gi 13375912 refNMO24660.1 Homo sapiens hypothetical protein FLJ22573 (FLJ22573); the human protein sequence corresponds to reference: Q9H665 ACCESSION:Q9H665 NID: Homo sapiens (Human). Hypothetical protein FLJ22573.

The mouse gene of interest is hypothetical protein MGC30332 (MGC30332), ortholog of human hypothetical protein FLJ22573 (FLJ22573).

FLJ22573 is a hypothetical type I plasma membrane protein, consisting of a signal peptide, an extracellular domain, a transmembrane segment, and a cytoplasmic domain. Currently, its function is unknown.

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 21 | 44 | 14 | 79 |
| Expected | 19.75 | 39.5 | 19.75 | 79 |

Chi-Sq.=0.75 Significance=0.6872893 (hom/n)=0.23 Avg. Litter Size=0

### Mutation Information

Mutation Type: Homologous Recombination (standard)
Description: Coding exons 1 through 4 were targeted (NCBI accession NM_{—}145580.1).
1. Wild-type Expression Panel: Expression of the target gene was detected, among 13 adult tissue samples tested by RT-PCR, in brain, spinal cord, thymus, spleen, and kidney.
2. QC Expression: Disruption of the target gene was confirmed by Southern hybridization analysis.

70.65.1. Phenotypic Analysis (for Disrupted Gene: DNA255219 (UNQ11632)

(a) Overall Phenotypic Summary:

Mutation of the gene encoding the ortholog of a human hypothetical membrane protein resulted in decreased microCT bone-related measurements. Gene disruption was confirmed by Southern blot.

(b) Bone Metabolism & Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Bone MicroCT Analysis:

Procedure: MicroCT was also used to get very sensitive measurements of BMD. One vertebra and 1 femur were taken from a cohort of 4 wild type and 8 homozygous mice. Measurements were taken of lumbar 5 vertebra trabecular bone volume, trabecular thickness, connectivity density and midshaft femur total bone area and cortical thickness. The μCT40 scans provided detailed information on bone mass and architecture. Multiple bones were placed into sample holders and scanned automatically. Instrument software was used to select regions of interest for analysis. Trabecular bone parameters were analyzed in the fifth lumbar vertebrae (LV5) at 16 micrometer resolution and cortical bone parameters were analyzed in the femur midshaft at a resolution of 20 micrometers.

Results:

MicroCT: The male (−/−) knockouts exhibited decreased trabecular number and connectivity density when compared with their gender-matched wildtype littermates and the historical means.

The (−/−) mice analyzed by bone micro CT analysis exhibited decreased bone measurements when compared with their (+/+) littermates, suggestive of abnormal bone disorders. The (−/−) mice exhibited a negative bone phenotype with abnormal and decreased bone measurements reflective of bone metabolic disorders. The negative bone phenotype indicates that PRO50298polypeptides or agonists thereof would be useful for maintaining bone homeostasis. In addition, PRO50298polypeptides would be useful in bone healing or for the treatment of arthritis or osteoporosis, whereas antagonists (or inhibitors) of PRO50298 polypeptides or its encoding gene would lead to abnormal or pathological bone disorders including inflammatory diseases associated with abnormal bone metabolism including arthritis, osteoporosis and osteopenia.

70.66. Generation and Analysis of Mice Comprising DNA256561 (UNQ12179) Gene Disruptions

In these knockout experiments, the gene encoding PRO51592 polypeptides (designated as DNA256561) (UNQ12179) was disrupted. The gene specific information for these studies is as follows: the mutated mouse gene corresponds to nucleotide reference: NM_{—}019465 ACCESSION:NM_{—}019465 NID:9506516 Mus musculus Mus musculus cytotoxic and regulatory T cell molecule (Crtam); protein reference: Q9Z151 ACCESSION:Q9Z151 NID: Mus musculus (Mouse). CLASS I MHC-RESTRICTED T CELL ASSOClATED MOLECULE; the human gene sequence reference: NM_{—}019604 Homo sapiens class-I MHC-restricted T cell associated molecule (CRTAM); the human protein sequence corresponds to reference: 095727 ACCESSION:095727 NID: Homo sapiens (Human). Class-I MHC-restricted T cell associated molecule.

The mouse gene of interest is Crtam (cytotoxic and regulatory T cell molecule), ortholog of human CRTAM (class-I MHC-restricted T cell associated molecule). Aliases include class I-restricted T cell-associated molecule.

CRTAM is a type I plasma membrane protein expressed in T cells that likely functions as a receptor or cell adhesion molecule. The protein consists of a signal peptide, an immunoglobulin-like domain (Pfam accession PF00047), a transmembrane segment, and a cytoplasmic C terminus. CRTAM is likely to play a role in immune function (Kennedy et al, J Leukoc Biol 67(5):725-34 (2000); Du Pasquier et al, C R Biol 327(6):591-601 (2004)).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 20 | 38 | 23 | 81 |
| Expected | 20.25 | 40.5 | 20.25 | 81 |

Chi-Sq.=0.52 Significance=0.7710516 (hom/n)=0.25 Avg. Litter Size=8

### Mutation Information

Mutation Type: Homologous Recombination (standard)
Description: Coding exon 1 was targeted (NCBI accession NM_{—}019465.1).
1. Wild-type Expression Panel: Expression of the target gene was detected in thymus, spleen, and stomach, small intestine, and colon among the 13 adult tissue samples tested by RT-PCR.
2. QC Expression: Disruption of the target gene was confirmed by Southern hybridization analysis.

70.66.1. Phenotypic Analysis (for Disrupted Gene: DNA256561 (UNQ12179)

(a) Overall Phenotypic Summary:

Mutation of the gene encoding the ortholog of human class-I MHC-restricted T cell associated molecule (CRTAM) resulted in thymic T cell lymphoma in one (−/−) mouse. In addition, several heterozygous (+/−) and homozygous (−/−) mice exhibited cataracts of differing severity. Improved glucose tolerance was also noted in the (−/−) mice. Gene disruption was confirmed by Southern blot.

(b) Pathology:

Microscopic Observations: Of the 6 (−/−) mice examined, 1 exhibited a lymphocytic malignant lymphoma in the thymus that effaced the thymic cortex and invaded locally into the mediastinum and lung. Lymph nodes and spleen were negative for neoplastic cells in the affected mouse. Although this T-cell lymphoma was present in only 1/6 (−/−) mice, it is a rare tumor in mice of this age and is very likely gene-related.
Gene Expression LacZ activity was not detected in the panel of tissues by immunohistochemical analysis.

(c) Cardiovascular Phenotypic Analysis:

In the area of cardiovascular biology, phenotypic testing was performed to identify potential targets for the treatment of cardiovascular, endothelial or angiogenic disorders. One such phenotypic test included optic fundus photography and angiography to determine the retinal arteriovenous ratio (A/V ratio) in order to flag various eye abnormalities. An abnormal A/V ratio signals such systemic diseases or disorders that may be related to the vascular disease of hypertension (and any disease that causes hypertension, e.g. atherosclerosis), diabetes or other ocular diseases corresponding to ophthalmological disorders. Such eye abnormalities may include but are not limited to the following: retinal abnormality is retinal dysplasia, various retinopathies, restenosis, retinal artery obstruction or occlusion; retinal degeneration causing secondary atrophy of the retinal vasculature, retinitis pigmentosa, macular dystrophies, Stargardt's disease, congenital stationary night blindness, choroideremia, gyrate atrophy, Leber's congenital amaurosis, retinoschisis disorders, Wagner's syndrome, Usher syndromes, Zellweger syndrome, Saldino-Mainzer syndrome, Senior-Loken syndrome, Bardet-Biedl syndrome, Alport's syndrome, Alstom's syndrome, Cockayne's syndrome, dysplaisa spondyloepiphysaria congentia, Flynn-Aird syndrome, Friedreich ataxia, Hallgren syndrome, Marshall syndrome, Albers-Schnoberg disease, Refsum's disease, Kearns-Sayre syndrome, Waardenburg's syndrome, Alagile syndrome, myotonic dystrophy, olivopontocerebellar atrophy, Pierre-Marie dunsdrome, Stickler syndrome, carotinemeia, cystinosis, Wolfram syndrome, Bassen-Kornzweig syndrome, abetalipoproteinemia, incontinentia pigmenti, Batten's disease, mucopolysaccharidoses, homocystinuria, or mannosidosis.

Procedure: A cohort of 4 wild type, 4 heterozygous and 8 homozygous mice were tested in this assay. Optic fundus photography was performed on conscious animals using a Kowa Genesis small animal fundus camera modified according to Hawes and coauthors (Hawes et al., 1999 Molecular Vision 1999; 5:22). Intra-peritoneal injection of fluorescein permitted the acquisition of direct light fundus images and fluorescent angiograms for each examination. In addition to direct ophthalmological changes, this test can detect retinal changes associated with systemic diseases such as diabetes and atherosclerosis or other retinal abnormalities. Pictures were provided of the optic fundus under normal light. The angiographic pictures allowed examination of the arteries and veins of the eye. In addition an artery to vein (A/V) ratio was determined for the eye.

Ophthalmology analysis was performed on generated F2 wild type, heterozygous, and homozygous mutant progeny using the protocol described above. Specifically, the A/V ratio was measured and calculated according to the fundus images with Kowa COMIT+ software. This test takes color photographs through a dilated pupil: the images help in detecting and classifying many diseases. The artery to vein ratio (A/V) is the ratio of the artery diameter to the vein diameter (measured before the bifurcation of the vessels). Many diseases will influence the ratio, i.e., diabetes, cardiovascular disorders, papilledema, optic atrophy or other eye abnormalities such as retinal degeneration (known as retinitis pigmentosa) or retinal dysplasia, vision problems or blindness. Thus, phenotypic observations which result in an increased artery-to-vein ratio in homozygous (−/−) and heterozygous (+/−) mutant progeny compared to wildtype (+/+) littermates would be indicative of such pathological conditions.

Results:

Fundus: Among the (−/−) and (+/−) mice analyzed, several mice exhibited cataracts of differing severity.

In summary, by knocking out the gene identified as DNA256561 (UNQ12179) which encodes PRO51592 polypeptides, the homozygous mutant progeny exhibit phenotypes which are associated with cataract formation and/or other opthalmological disorders. Such detected ophthalmology changes are most commonly associated with cardiovascular systemic diseases. In particular, cataract formation may be indicative of a cardiovascular complication related to disturbances in the blood coagulation cascade. Cataracts are also associated with such systemic diseases as: Human Down's syndrome, Hallerman-Streiff syndrome, Lowe syndrome, galactosemia, Marfan syndrome, Trismoy 13-15 condition, Alport syndrome, myotonic dystrophy, Fabry disease, hypothroidisms, Conradi syndrome. Thus, antagonists of PRO51592 encoding genes would lead to similar pathological changes, whereas agonists would be useful as therapeutic agents in the prevention of cataract formation and/or the underlying cardiovascular disease or opthalmological disorders.

(d) Phenotypic Analysis: Metabolism-Blood Chemistry/Glucose Tolerance

In the area of metabolism, targets may be identified for the treatment of diabetes. Blood chemistry phenotypic analysis includes blood glucose measurements. The COBAS Integra 400 (mfr: Roche) was used for running blood chemistry tests on the mice. In the area of metabolism, targets may be identified for the treatment of diabetes. Blood chemistry phenotypic analysis includes glucose tolerance tests to measure insulin sensitivity and changes in glucose metabolism. Abnormal glucose tolerance test results may indicate but may not be limited to the following disorders or conditions: Diabetes Type 1 and Type 2, Syndrome X, various cardiovascular diseases and/or obesity.

Procedure: A cohort of 2 wild type and 4 homozygous mice were used in this assay. The glucose tolerance test is the standard for defining impaired glucose homeostasis in mammals. Glucose tolerance tests were performed using a Lifescan glucometer. Animals were injected IP at 2 g/kg with D-glucose delivered as a 20% solution and blood glucose levels were measured at 0, 30, 60 and 90 minutes after injection.

Results:

Glucose Tolerance Test: The male mutant (−/−) mice tested exhibited enhanced glucose tolerance when compared with their gender-matched (+/+) littermates.

In these studies the mutant (−/−) mice showed an increased or enhanced glucose tolerance in the presence of normal fasting glucose at all 3 intervals tested when compared with their gender-matched (+/+) littermates and the historical means. In addition, hyperinsulinemia was not apparent in the (−/−) mice. Thus, knockout mice exhibited an increased insulin sensitivity or the opposite phenotypic pattern of an impaired glucose homeostasis.

(e) Additional Studies

UNQ12179 appears to be involved in tissue-selective trafficking of immune cells in the lymph nodes. Hyperproliferation of effectory/memory CD4 T cells occurs in the knockout animals after TcR stimulation (anti-CD3 plus anti-CD28).

70.67. Generation and Analysis of Mice Comprising DNA76398-1699 (UNQ830) Gene Disruptions

In these knockout experiments, the gene encoding PRO1757 polypeptides (designated as DNA76398-1699) (UNQ830) was disrupted. The gene specific information for these studies is as follows: the mutated mouse gene corresponds to nucleotide reference: AK014261 Mus musculus 13 days embryo head cDNA, RIKEN full-length enriched library, clone:3110079015 product:hypothetical protein, full insert sequence; protein reference: Q9CXL7 ACCESSION:Q9CXL7 NID: Mus musculus (Mouse). 3110079015Rik protein; the human gene sequence reference: NM206895 Homo sapiens ASCL830 (UNQ830); the human protein sequence corresponds to reference: Q6UX34 ACCESSION:Q6UX34 NID: Homo sapiens (Human). ASCL830.

The mouse gene of interest is RIKEN cDNA 3110079015 gene, ortholog of human ASCL830 (UNQ830). ASCL830 is a likely type I plasma membrane protein, consisting of a signal peptide, an extracellular domain, a transmembrane segment, and a short C-terminal domain. The function of this protein is not known.

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 18 | 38 | 4 | 60 |
| Expected | 15 | 30 | 15 | 60 |

Chi-Sq.=15.11 Significance=5.2348623E-4 (hom/n)=0.1 Avg. Litter Size=6

### Mutation Information

Mutation Type: Homologous Recombination (standard)
Description: Coding exons 2 and 3 were targeted (NCBI accession AK014261.1).
1. Wild-type Expression Panel: Expression of the target gene was detected only in brain and spinal cord among the 13 adult tissue samples tested by RT-PCR.
2. QC Expression: Disruption of the target gene was confirmed by Southern hybridization analysis.

70.67.1. Phenotypic Analysis (for Disrupted Gene: DNA76398-1699 (UNQ830)

(a) Overall Phenotypic Summary:

Mutation of the gene encoding the ortholog of a human hypothetical membrane protein resulted in reduced viability of (−/−) mutants. Surviving male (−/−) mice exhibited a decreased serum triglyceride and cholesterol level, as well as decreased serum glucose levels. The surviving female (−/−) mice exhibited decreased activity during home-cage activity testing. Gene disruption was confirmed by Southern blot.

(b) Pathology

Microscopic Observations: An examination at gestation day 18 revealed 4 (−/−) embryos, 11 (+/−) embryos, and 3 (+/+) embryos, for a total of 18 embryos. Histologic examination of the 4 (−/−) embryos and 2 (+/+) embryos revealed no notable differences. Mendelian numbers of homozygotes are present at embryonic day 18.5d and histologically look normal. However, only a third of the expected number of homozygotes survive at six weeks. Gene Expression: LacZ activity was not detected in the panel of tissues by immunohistochemical analysis.

(c) Phenotypic Analysis: CNS/Neurology

In the area of neurology, analysis focused herein on identifying in vivo validated targets for the treatment of neurological and psychiatric disorders including depression, generalized anxiety disorders, attention deficit hyperactivity disorder, obsessive compulsive disorder, schizophrenia, cognitive disorders, hyperalgesia and sensory disorders. Neurological disorders include the category defined as “anxiety disorders” which include but are not limited to: mild to moderate anxiety, anxiety disorder due to a general medical condition, anxiety disorder not otherwise specified, generalized anxiety disorder, panic attack, panic disorder with agoraphobia, panic disorder without agoraphobia, posttraumatic stress disorder, social phobia, specific phobia, substance-induced anxiety disorder, acute alcohol withdrawal, obsessive compulsive disorder, agoraphobia, bipolar disorder I or II, bipolar disorder not otherwise specified, cyclothymic disorder, depressive disorder, major depressive disorder, mood disorder, substance-induced mood disorder. In addition, anxiety disorders may apply to personality disorders including but not limited to the following types: paranoid, antisocial, avoidant behavior, borderline personality disorders, dependent, histronic, narcissistic, obsessive-compulsive, schizoid, and schizotypal.

Procedure:

Behavioral screens were performed on a cohort of wild type, heterozygous and homozygous mice. All behavioral tests were done between 12 and 16 weeks of age unless reduced viability necessitates earlier testing.

Circadian Test Description:

Female mice are individually housed at 4 pm on the first day of testing in 48.2 cm×26.5 cm home cages and administered food and water ad libitum. Animals are exposed to a 12-hour light/dark cycle with lights turning on at 7 am and turning off at 7 pm. The system software records the number of beam interruptions caused by the animal's movements, with beam breaks automatically divided into ambulations. Activity is recorded in 60, one-hour intervals during the three-day test. Data generated are displayed by median activity levels recorded for each hour (circadian rhythm) and median total activity during each light/dark cycle (locomotor activity) over the three-day testing period.

Results:

The female (−/−) mice exhibited decreased median ambulatory counts during the 12-hour habituation period of home-cage activity testing when compared with their gender-matched (+/+) littermates. Decreased rearing in open field testing was also observed for the homozygotes. Both observations are indicative of hypoactivity in the (−/−) mice.

These results are indicative of a marked hypo-locomotor activity in the (−/−) mice consistent with lethargy or depressive disorders. Antagonists or inhibitors of PRO1757 polypeptides or the PRO1757 encoding gene would be expected to mimic this behavior. Likewise, PRO1757 polypeptides or agonists thereof, would be useful in the treatment of such neurological disorders including depressive disorders or other decreased anxiety-like symptoms such as lethargy, cognitive disorders, hyperalgesia and sensory disorders.

(d) Phenotypic Analysis: Cardiology

In the area of cardiovascular biology, targets were identified herein for the treatment of hypertension, atherosclerosis, heart failure, stroke, various coronary artery diseases, dyslipidemias such as high cholesterol (hypercholesterolemia) and elevated serum triglycerides (hypertriglyceridemia), diabetes and/or obesity. The phenotypic tests included the measurement of serum cholesterol and triglycerides.

Blood Lipids

Procedure: A cohort of wild type, heterozygous and homozygous mice were tested in this assay. High cholesterol levels and increased triglyceride blood levels are recognized risk factors in the development of cardiovascular disease and/or diabetes. Measuring blood lipids facilitates the finding of biological switches that regulate blood lipid levels Inhibition of factors which elevate blood lipid levels may be useful for reducing the risk for cardiovascular disease. In these blood chemistry tests, measurements were recorded using the COBAS Integra 400 (mfr: Roche).

Results:

The male (−/−) mice exhibited notably decreased mean serum triglyceride and cholesterol levels when compared with their gender-matched (+/+) littermates and the historical mean.

Thus, mutant mice deficient in the PRO1757 encoding gene can serve as a model for treatment of cardiovascular disease especially those diseases which are associated with dyslipidemia. PRO1757 polypeptides or its encoding gene would be useful in regulating blood lipids and in particular for maintaining normal levels of triglycerides.

(e) Phenotypic Analysis: Metabolism-Blood Chemistry

In the area of metabolism, targets may be identified for the treatment of diabetes. Blood chemistry phenotypic analysis includes blood glucose measurements. The COBAS Integra 400 (mfr: Roche) was used for running blood chemistry tests on the mice. In the area of metabolism, targets may be identified for the treatment of diabetes.

Results:

The male (−/−) mice exhibited decreased mean serum glucose levels when compared with their gender-matched (+/+) littermates and the historical means.

70.68. Generation and Analysis of Mice Comprising DNA96879-2619 (UNQ1938) Gene Disruptions

In these knockout experiments, the gene encoding PRO4421 polypeptides (designated as DNA96879-2619) (UNQ1938) was disrupted. The gene specific information for these studies is as follows: the mutated mouse gene corresponds to nucleotide reference: NM_{—}029612 ACCESSION:NM_{—}029612 NID:20514783 Mus musculus Mus musculus CD2 antigen family, member 10 (Cd2f10-pending); protein reference:Q9D780 ACCESSION:Q9D780 NID: Mus musculus (Mouse). 2310026104RIK PROTEIN; the human gene sequence reference: NM_{—}033438 ACCESSION:NM_{—}033438 NID:15559204 Homo sapiens Homo sapiens CD84-H1 precursor (CD84-H1); the human protein sequence corresponds to reference: Q96A28 ACCESSION: Q96A28 NID: Homo sapiens (Human). CD84-H1 (CD2 FAMILY 10).

The mouse gene of interest is Slamf9 (SLAM family member 9), ortholog of human SLAMF9. Aliases include Cd2f10, SF2001, CD2F-10, CD84-H1, 2310026104Rik, PRO4421, and CD2 antigen family member 10. SLAMF9 is an integral plasma membrane protein that likely functions as an adhesion molecule or receptor. The protein consists of a signal peptide, an extracellular Ig-like domain, a transmembrane segment, and a short cytoplasmic C terminus. SLAMF9 is a homolog of CD2 family members, which function as coreceptors for lymphocyte activation or adhesion. SLAMF9 is expressed in spleen, lung, testis, and a macrophage cell line but not in peripheral blood leukocytes (Fennelly et al, Immunogenetics 53(7):599-602 (2001)).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 20 | 31 | 12 | 63 |
| Expected | 15.75 | 31.5 | 15.75 | 63 |

Chi-Sq.=4.48 Significance=0.1064585 (hom/n)=0.2 Avg. Litter Size=7

### Mutation Information

Mutation Type: Homologous Recombination (standard)
Description: Coding exons 1 through 3 were targeted (NCBI accession NM_{—}029612.2).
1. Wild-type Expression Panel: Expression of the target gene was detected in all 13 adult tissue samples tested by RT-PCR, except brain, skeletal muscle, and bone.
2. QC Expression: Disruption of the target gene was confirmed by Southern hybridization analysis.

70.68.1. Phenotypic Analysis (for Disrupted Gene: DNA96879-2619 (UNQ1938)

(a) Overall Phenotypic Summary:

Mutation of the gene encoding the ortholog of human SLAM family member 9 (SLAMF9) resulted in increased total tissue mass, lean body mass, percent total body fat and fat mass (g) in the homozygotes (−/−). In addition, the (−/−) mice exhibited increase bone-related measurements. Both male and female (−/−) mice exhibited increased triglyceride levels, whereas male (−/−) mice also showed elevated levels of mean serum cholesterol. Gene disruption was confirmed by Southern blot.

(b) Phenotypic Analysis: Cardiology

In the area of cardiovascular biology, targets were identified herein for the treatment of hypertension, atherosclerosis, heart failure, stroke, various coronary artery diseases, dyslipidemias such as high cholesterol (hypercholesterolemia), diabetes and/or obesity. The phenotypic tests included the measurement of serum cholesterol.

Blood Lipids

Procedure: A cohort of 4 wild type, 4 heterozygous and 8 homozygous mice were tested in this assay. High cholesterol levels are recognized risk factors in the development of cardiovascular disease and/or diabetes. Measuring blood lipids facilitates the finding of biological switches that regulate blood lipid levels Inhibition of factors which elevate blood lipid levels may be useful for reducing the risk for cardiovascular disease. In these blood chemistry tests, measurements were recorded using the COBAS Integra 400 (mfr: Roche).

Results:

Blood Chemistry: The male (−/−) mice exhibited an increased mean serum cholesterol level when compared with their gender-matched (+/+) littermates and the historical mean. Both the male and female knockout mice (−/−) also exhibited increased mean serum triglyceride levels compared to t heir (+/+) littermates.

As summarized above, the (−/−) mice exhibited notably increased mean serum cholesterol and triglyceride levels when compared with their gender-matched (+/+) littermates and the historical means. Thus, mutant mice deficient in the PRO4421 gene may serve as a model for cardiovascular disease. PRO4421 polypeptides or its encoding gene would be useful in regulating blood lipids such as triglycerides. Thus, PRO14421 polypeptides or agonists thereof would be useful in the treatment of such cardiovascular diseases as hypertension, atherosclerosis, heart failure, stroke, various coronary diseases, hypercholesterolemia, hypertriglyceridemia, diabetes and/or obesity.

(c) Bone Metabolism & Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Results:

DEXA: The female (−/−) mice exhibited increased mean total tissue mass, lean body mass, percent total body fat, volumetric bone mineral density, and total body bone mineral density and fat (g) when compared with their gender-matched (+/+) littermates and the historical means. The male (−/−) mice also exhibited increased total tissue mass, lean body mass, and total body fat.

The (−/−) mice exhibited increased total tissue mass and mean total body fat as well as increased bone mineral density measurements when compared with their gender-matched (+/+) littermates. These results indicate that the knockout mutant phenotype is associated with such bone abnormalities as osteopetrosis. Osteopetrosis is a condition characterized by abnormal thickening and hardening of bone and abnormal fragility of the bones. As such, PRO4421 polypeptides or agonists thereof would be beneficial for the treatment of osteopetrosis. A phenotype associated with an increased bone mineral content, and total body and femoral bone mineral density suggests that agents which mimic these effects (e.g. antagonists of PRO4421 polypeptides) would be useful in bone healing. In addition, female mutant (−/−) mice also exhibited an increased mean percentage of body fat (as well as increased triglyceride and cholesterol levels) suggestive of an obesity phenotype. These observations suggest that mutant mice deficient in the gene which encodes PRO4421 polypeptides leads to metabolic disorders associated with accumulation of fat (dyslipidemia) but also abnormal bone measurements reflective of general metabolic disorders which can be associated with obesity. Thus, PRO4421 polypeptides or agonists thereof would be useful in the treatment or prevention of such disorders as obesity or other metabolic diseases.

70.69. Generation and Analysis of Mice Comprising DNA119516-2797 (UNQ3071) Gene Disruptions

In these knockout experiments, the gene encoding PRO9903 polypeptides (designated as DNA119516-2797) (UNQ3071) was disrupted. The gene specific information for these studies is as follows: the mutated mouse gene corresponds to nucleotide reference: XM_{—}355322 PREDICTED: Mus musculus RIKEN cDNA 5930434B04 gene (5930434B04Rik); protein reference:Q8BG21 ACCESSION:Q8BG21 NID: Mus musculus (Mouse). Mus musculus 0 day neonate thymus cDNA, RIKEN full-length enriched library, clone:A430023D18 product:unknown EST, full insert sequence (Mus musculus 13 days embryo stomach cDNA, RIKEN full-length enriched library, clone: D530007N05 product:unknown EST, full insert sequence); the human gene sequence reference: NM_{—}017586 ACCESSION:NM_{—}017586 NID: gi 8922115 ref NM017586.1 Homo sapiens chromosome 9 open reading frame 7 (C9orf7); the human protein sequence corresponds to reference:Q9UGQ2 ACCESSION:Q9UGQ2 NID: Homo sapiens (Human). Hypothetical protein FLJ90371 (Chromosome 9 open reading frame 7) (Hypothetical protein NT2RP3001619).

The mouse gene of interest is RIKEN cDNA 5930434B04 gene, ortholog of human C9orf7 (chromosome 9 open reading frame 7). Aliases include D9S2135.

C9orf7 is a putative plasma membrane protein, consisting of a signal peptide and two transmembrane segments.

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 23 | 41 | 25 | 89 |
| Expected | 22.25 | 44.5 | 22.25 | 89 |

Chi-Sq.=1.62 Significance=0.44485807 (hom/n)=0.28 Avg. Litter Size=12

### Mutation Information

Mutation Type: Homologous Recombination (standard)
Description: Coding exon 1 was targeted (NCBI accession AK020041).
1. Wild-type Expression Panel: Expression of the target gene was detected in embryonic stem (ES) cells and in all 13 adult tissue samples tested by RT-PCR, except bone and stomach, small intestine, and colon.
2. QC Expression: Disruption of the target gene was confirmed by Southern hybridization analysis.

70.69.1. Phenotypic Analysis (for Disrupted Gene: DNA119516-2797 (UNQ3071)

(a) Overall Phenotypic Summary:

Mutation of the gene encoding the ortholog of human chromosome 9 open reading frame 7 (C9orf7) resulted in a hearing impairment in (−/−) mice. Both male and female (−/−) mice exhibited increased mean serum triglyceride levels, more notably in the females. Male (−/−) mice also exhibited increased mean serum cholesterol. Serum IgM and IgG2a levels were also decreased in the (−/−) knockout mice. Two female (−/−) mice showed myeloid hyperplasia in the bone marrow. The male (−/−) mice exhibited increased mean total tissue mass and lean body mass as well as increased mean femoral mid-shaft cortical thickness. Gene disruption was confirmed by Southern blot.

(b) Pathology:

Microscopic Observations: Both of the female (−/−) mice exhibited myeloid hyperplasia in the bone marrow. No notable difference was observed for the male (−/−) mice.
Gene Expression LacZ activity was not detected in the panel of tissues by immunohistochemical analysis.

(c) Phenotypic Analysis: Cardiology

In the area of cardiovascular biology, targets were identified herein for the treatment of hypertension, atherosclerosis, heart failure, stroke, various coronary artery diseases, dyslipidemias such as high cholesterol (hypercholesterolemia) and elevated serum triglycerides (hypertriglyceridemia), diabetes and/or obesity. The phenotypic tests included the measurement of serum cholesterol and triglycerides.

Blood Lipids

Procedure: A cohort of 4 wild type, 4 heterozygous and 8 homozygous mice were tested in this assay. High cholesterol levels and increased triglyceride blood levels are recognized risk factors in the development of cardiovascular disease and/or diabetes. Measuring blood lipids facilitates the finding of biological switches that regulate blood lipid levels Inhibition of factors which elevate blood lipid levels may be useful for reducing the risk for cardiovascular disease. In these blood chemistry tests, measurements were recorded using the COBAS Integra 400 (mfr: Roche).

Results:

The male (−/−) mice exhibited an increased mean serum cholesterol level when compared with their gender-matched (+/+) littermates and the historical mean. Both the male and female (−/−) mice exhibited increased mean serum triglyceride levels, the difference being more notable in the females.

As summarized above, the (−/−) mice exhibited notably increased mean serum cholesterol and triglyceride levels when compared with their gender-matched (+/+) littermates and the historical means. Thus, mutant mice deficient in the PRO9903 gene may serve as a model for cardiovascular disease. PRO9903 polypeptides or its encoding gene would be useful in regulating blood lipids such as triglycerides. Thus, PRO9903 polypeptides or agonists thereof would be useful in the treatment of such cardiovascular diseases as hypertension, atherosclerosis, heart failure, stroke, various coronary diseases, hypercholesterolemia, hypertriglyceridemia, diabetes and/or obesity.

(d) Immunology Phenotypic Analysis

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

T lymphocytes (T cells) are an important component of a mammalian immune response. T cells recognize antigens which are associated with a self-molecule encoded by genes within the major histocompatibility complex (MHC). The antigen may be displayed together with MHC molecules on the surface of antigen presenting cells, virus infected cells, cancer cells, grafts, etc. The T cell system eliminates these altered cells which pose a health threat to the host mammal. T cells include helper T cells and cytotoxic T cells. Helper T cells proliferate extensively following recognition of an antigen-MHC complex on an antigen presenting cell. Helper T cells also secrete a variety of cytokines, i.e., lymphokines, which play a central role in the activation of B cells, cytotoxic T cells and a variety of other cells which participate in the immune response.

In many immune responses, inflammatory cells infiltrate the site of injury or infection. The migrating cells may be neutrophilic, eosinophilic, monocytic or lymphocytic as can be determined by histologic examination of the affected tissues. Current Protocols in Immunology, ed. John E. Coligan, 1994, John Wiley & Sons, Inc.

Many immune related diseases are known and have been extensively studied. Such diseases include immune-mediated inflammatory diseases (such as rheumatoid arthritis, immune mediated renal disease, hepatobiliary diseases, inflammatory bowel disease (IBD), psoriasis, and asthma), non-immune-mediated inflammatory diseases, infectious diseases, immunodeficiency diseases, neoplasia, and graft rejection, etc. In the area of immunology, targets were identified for the treatment of inflammation and inflammatory disorders.

In the area of immunology, targets have been identified herein for the treatment of inflammation and inflammatory disorders. Immune related diseases, in one instance, could be treated by suppressing the immune response. Using neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

The following test was performed:

Serum Immunoglobulin Isotyping Assay:

The Serum Immunoglobulin Isotyping Assay is performed using a Cytometric Bead Array (CBA) kit. This assay is used to rapidly identify the heavy and light chain isotypes of a mouse monoclonal antibody in a single sample. The values expressed are “relative fluorescence units” and are based on the detection of kappa light chains. Any value <6 is not significant.

Results:

Serum Immunoglobulins: The (−/−) mice exhibited decreased mean serum IgM levels when compared with that of their (+/+) littermates, the (+/+) mice for the project run, and the historical median.

Mutant (−/−) mice exhibited decreased IgM serum immunoglobulins compared to their gender-matched (+/+) littermates. IgM immunoglobulins are the first to be produced in a humoral immune response for neutralization of bacterial toxins and are particularly important in activating the complement system. The observed phenotype suggests that the PRO9903 polypeptide is a regulator of inflammatory responses. These immunological abnormalities suggest that PRO9903 polypeptides or agonists thereof would be important agents which would stimulate the immune system (such as T cell proliferation) and would find utility in the cases wherein this effect would be beneficial to the individual such as in the case of leukemia, and other types of cancer, and in immunocompromised patients, such as AIDS sufferers. Accordingly, antagonists (or inhibitors) of PRO9903 polypeptides would be useful in inhibiting the immune response and would be useful candidates for suppressing harmful immune responses, e.g. in the case of graft rejection or graft-versus-host diseases.

(e) Phenotypic Analysis: CNS/Neurology

In the area of neurology, analysis focused herein on identifying in vivo validated targets for the treatment of neurological and psychiatric disorders including depression, generalized anxiety disorders, attention deficit hyperactivity disorder, obsessive compulsive disorder, schizophrenia, cognitive disorders, hyperalgesia and sensory disorders. Neurological disorders include the category defined as “anxiety disorders” which include but are not limited to: mild to moderate anxiety, anxiety disorder due to a general medical condition, anxiety disorder not otherwise specified, generalized anxiety disorder, panic attack, panic disorder with agoraphobia, panic disorder without agoraphobia, posttraumatic stress disorder, social phobia, specific phobia, substance-induced anxiety disorder, acute alcohol withdrawal, obsessive compulsive disorder, agoraphobia, bipolar disorder I or II, bipolar disorder not otherwise specified, cyclothymic disorder, depressive disorder, major depressive disorder, mood disorder, substance-induced mood disorder. In addition, anxiety disorders may apply to personality disorders including but not limited to the following types: paranoid, antisocial, avoidant behavior, borderline personality disorders, dependent, histronic, narcissistic, obsessive-compulsive, schizoid, and schizotypal.

Procedure:

Behavioral screens were performed on a cohort of 4 wild type, 4 heterozygous and 8 homozygous mice. All behavioral tests were done between 12 and 16 weeks of age unless reduced viability necessitates earlier testing. These tests included open field to measure anxiety, activity levels and exploration.

Prepulse Inhibition of the Acoustic Startle Reflex

Prepulse inhibition of the acoustic startle reflex occurs when a loud 120 decibel (dB) startle-inducing tone is preceded by a softer (prepulse) tone. The PPI paradigm consists of six different trial types (70 dB background noise, 120 dB alone, 74 dB+120 dB−pp 4, 78 dB+120 dB−pp 8, 82 dB+120 dB−pp 12, and 90 dB+120 dB−pp 20) each repeated in pseudorandom order six times for a total of 36 trials. The max response to the stimulus (V max) is averaged for each trial type. Animals with a 120 dB average value equal to or below 100 are excluded from analysis. The percent that the prepulse inhibits the animal's response to the startle stimulus is calculated and graphed.

Results:

Sensorimotor Gating/Attention: Of the 8 (−/−) mice tested, only 1 exhibited a startle response, suggesting hearing impairment in the mutants.

(f) Bone Metabolism & Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Bone MicroCT Analysis:

Procedure: MicroCT was also used to get very sensitive measurements of BMD. One vertebra and 1 femur were taken from a cohort of 4 wild type and 8 homozygous mice. Measurements were taken of lumbar 5 vertebra trabecular bone volume, trabecular thickness, connectivity density and midshaft femur total bone area and cortical thickness. The μCT40 scans provided detailed information on bone mass and architecture. Multiple bones were placed into sample holders and scanned automatically. Instrument software was used to select regions of interest for analysis. Trabecular bone parameters were analyzed in the fifth lumbar vertebrae (LV5) at 16 micrometer resolution and cortical bone parameters were analyzed in the femur midshaft at a resolution of 20 micrometers.

Results:

1. DEXA: The male (−/−) mice exhibited increased mean total tissue mass and lean body mass when compared with their gender-matched (+/+) littermates and the historical means.
2. Micro-CT: The male (−/−) mice exhibited increased mean femoral mid-shaft cortical thickness when compared with their gender-matched (+/+) littermates and the historical mean.

In summary, the (−/−) mice exhibited increased mean total tissue mass and lean body mass as well as increased femoral mid-shaft cortical thickness when compared with their gender-matched (+/+) littermates. These results indicate that the knockout mutant phenotype is associated with such bone abnormalities as osteopetrosis. Osteopetrosis is a condition characterized by abnormal thickening and hardening of bone and abnormal fragility of the bones. As such, PRO9903 polypeptides or agonists thereof would be beneficial for the treatment of osteopetrosis or other osteo-related diseases. On the other hand, inhibitors or antagonists of PRO9903 polypeptides would be useful in bone healing. The noted increase in total tissue mass and lean body mass in the mutant (−/−) mice is associated with an obesity phenotype. Thus, PRO9903 polypeptides or agonists thereof would be useful in the treatment of dyslipidemia associated with obesity.

70.70. Generation and Analysis of Mice Comprising DNA59609-1470 (UNQ549) Gene Disruptions

In these knockout experiments, the gene encoding PRO1106 polypeptides (designated as DNA59609-1470) (UNQ549) was disrupted. The gene specific information for these studies is as follows: the mutated mouse gene corresponds to nucleotide reference: NM_{—}146118 Mus musculus solute carrier family 25 (mitochondrial carrier, phosphate carrier), member 25 (Slc25a25); protein reference: Q8JZT8 ACCESSION:Q8JZT8 NID: Mus musculus (Mouse). Mitochondrial Ca2-dependent solute carrier; the human gene sequence reference: NM_{—}052901 Homo sapiens solute carrier family 25 (mitochondrial carrier; phosphate carrier), member 25 (SLC25A25); the human protein sequence corresponds to reference:. Q705K2 ACCESSION:Q705K2 NID: Homo sapiens (Human). Mitochondrial ATP-Mg/Pi carrier (Small calcium-binding mitochondrial carrier 2).

The mouse gene of interest is Slc25a25 (solute carrier family 25 [mitochondrial carrier, phosphate carrier], member 25), ortholog of human SLC25A25. Aliases include MCSC; MGC36388; mKIAA1896; 1110030N17Rik; PCSCL; SCAMC-2; mitochondrial Ca2-dependent solute carrier; solute carrier family 25 [mitochondrial carrier; phosphate carrier], member 25; and short calcium-binding mitochondrial carrier 2.

SLC25A25 is a calcium-dependent transporter located in the inner membrane of mitochondria that shuttles magnesium-ATP between mitochondria and cytosol in exchange for phosphate. SLC25A25 is expressed primarily in liver and skeletal muscle (Mashima et al, J Biol Chem 278(11):9520-7 (2003); del Arco and Satrustegui, J Biol Chem 279(23):24701-13 (2004); Fiermonte et al, J Biol Chem 279(29):30722-30 (2004)).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 20 | 31 | 21 | 72 |
| Expected | 18 | 36 | 18 | 72 |

Chi-Sq.=3.25 Significance=0.19691168 (hom/n)=0.23 Avg. Litter Size=7

### Mutation Information

Mutation Type: Hornologous Recornbination (standard)
Description: Coding exons 1 through 3 were targeted (NCBI accession BC019978.1).
1. Wild-type Expression Panel: Expression of the target gene was detected in ernbryonic stern (ES) cells and in all 13 adult tissue sarnples tested by RT-PCR, except bone.
2. QC Expression: Disruption of the target gene was confirmed by Southern hybridization analysis.

70.70.1. Phenotypic Analysis (for Disrupted Gene: DNA59609-1470 (UNQ549)

(a) Overall Phenotypic Summary:

Mutation of the gene encoding the ortholog of human solute carrier family 25 (mitochondrial carrier, phosphate carrier), member 25 (SLC25A25) resulted in the observation of decreased body length in the female (−/−) mice. Gene disruption was confirmed by Southern blot

(b) Bone Metabolism & Body Diagnostics

(1) Tissue Mass & Lean Body Mass Measurements—Dexa

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in total tissue mass (TTM).

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI, i.e., whole body, vertebrae, and both femurs).

Body Measurements (Body Length & Weight):

Body Measurements: A measurement of body length and weight was performed at approximately 16 weeks of age.

Results:

The female (−/−) mice exhibited decreased mean body length when compared with their gender-matched (+/+) littermates and the historical means. These results demonstrate a negative phenotype associated with knocking out the PRO1106 gene resulting in abnormal growth. Thus, PRO1106 polypeptides and agonists thereof would be useful in maintaining normal growth metabolism, whereas antagonists (or inhibitors) of PRO1106 polypeptides would mimic the negative growth related phenotype.

70.71. Generation and Analysis of Mice Comprising DNA59212-1627 (UNQ729) Gene Disruptions

In these knockout experiments, the gene encoding PRO1411 polypeptides (designated as DNA59212-1627) (UNQ729) was disrupted. The gene specific information for these studies is as follows: the mutated mouse gene corresponds to nucleotide reference: AY444557 Mus musculus epidermis-specific secreted protein SK89 precursor, mRNA, complete cds, alternatively spliced; protein reference: Q6SZJ9 ACCESSION:Q6SZJ9 NID: Mus musculus (Mouse). Epidermis-specific secreted protein SK89 precursor; the human gene sequence reference: NM_{—}033317 Homo sapiens dermokine (ZD52F10); the human protein sequence corresponds to reference: Q6E0U4 ACCESSION:Q6E0U4 NID: Homo sapiens (Human). Dermokine-beta.

The mouse gene of interest is RIKEN cDNA 1110014F24 gene, ortholog of human ZD52F10 (dermokine). Aliases include Dmkn, SK30, SK89, C130074A08, UNQ729, and epidermis-specific secreted protein.

ZD52F10 is a secreted protein expressed primarily in epidermal keratinocytes. Induction of ZD52F10 expression occurs in differentiating human keratinocytes in culture (Moffatt et al, Gene 344:123-31 (2004); Matsui et al, Genomics 84(2):384-97 (2004)).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 27 | 30 | 15 | 72 |
| Expected | 18 | 36 | 18 | 72 |

Chi-Sq.=4.48 Significance=0.1064585 (hom/n)=0.22 Avg. Litter Size=0

### Mutation Information

Mutation Type: Homologous Recombination (standard)
Description: Coding exons 1 through 4 were targeted (NCBI accession NM_{—}172899.1).
1. Wild-type Expression Panel: Expression of the target gene was detected in embryonic stem (ES) cells and in all 13 adult tissue samples tested by RT-PCR, except liver, skeletal muscle, and bone.
2. QC Expression: Disruption of the target gene was confirmed by Southern hybridization analysis.

70.71.1. Phenotypic Analysis (for Disrupted Gene: DNA59212-1627 (UNQ729)

(a) Overall Phenotypic Summary:

Mutation of the gene encoding the ortholog of human dermokine (ZD52F10) resulted in increased mean serum IgG2a levels in the (−/−) mice. Female homozygous (−/−) mice also exhibited an increased mean systolic blood pressure. Male (−/−) mice exhibited an impaired glucose tolerance. Gene disruption was confirmed by Southern blot.

(b) Immunology Phenotypic Analysis

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

T lymphocytes (T cells) are an important component of a mammalian immune response. T cells recognize antigens which are associated with a self-molecule encoded by genes within the major histocompatibility complex (MHC). The antigen may be displayed together with MHC molecules on the surface of antigen presenting cells, virus infected cells, cancer cells, grafts, etc. The T cell system eliminates these altered cells which pose a health threat to the host mammal. T cells include helper T cells and cytotoxic T cells. Helper T cells proliferate extensively following recognition of an antigen-MHC complex on an antigen presenting cell. Helper T cells also secrete a variety of cytokines, i.e., lymphokines, which play a central role in the activation of B cells, cytotoxic T cells and a variety of other cells which participate in the immune response.

In many immune responses, inflammatory cells infiltrate the site of injury or infection. The migrating cells may be neutrophilic, eosinophilic, monocytic or lymphocytic as can be determined by histologic examination of the affected tissues. Current Protocols in Immunology, ed. John E. Coligan, 1994, John Wiley & Sons, Inc.

Many immune related diseases are known and have been extensively studied. Such diseases include immune-mediated inflammatory diseases (such as rheumatoid arthritis, immune mediated renal disease, hepatobiliary diseases, inflammatory bowel disease (IBD), psoriasis, and asthma), non-immune-mediated inflammatory diseases, infectious diseases, immunodeficiency diseases, neoplasia, and graft rejection, etc. In the area of immunology, targets were identified for the treatment of inflammation and inflammatory disorders.

In the area of immunology, targets have been identified herein for the treatment of inflammation and inflammatory disorders. Immune related diseases, in one instance, could be treated by suppressing the immune response. Using neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

The following test was performed:

Serum Immunoglobulin Isotyping Assay:

The Serum Immunoglobulin Isotyping Assay is performed using a Cytometric Bead Array (CBA) kit. This assay is used to rapidly identify the heavy and light chain isotypes of a mouse monoclonal antibody in a single sample. The values expressed are “relative fluorescence units” and are based on the detection of kappa light chains. Any value <6 is not significant.

Results:

The (−/−) mice exhibited increased mean serum IgG2a levels when compared to their gender-matched littermate controls.

Mutant (−/−) mice exhibited elevation of IgG2a serum immunoglobulins compared to their gender-matched (+/+) littermates. IgG2a immunoglobulins have neutralization effects and to a lesser extent are important for activation of the complement system. The observed phenotype suggests that the PRO1411 polypeptide is a negative regulator of inflammatory responses. These immunological abnormalities suggest that inhibitors (antagonists) of PRO1411 polypeptides would be important agents which could stimulate the immune system (such as T cell proliferation) and would find utility in the cases wherein this effect would be beneficial to the individual such as in the case of leukemia, and other types of cancer, and in immunocompromised patients, such as AIDS sufferers. Accordingly, PRO1411 polypeptides or agonists thereof would be useful in inhibiting the immune response and would be useful candidates for suppressing harmful immune responses, e.g. in the case of graft rejection or graft-versus-host diseases.

(c) Diagnostics—Blood Pressure

Description:

Systolic blood pressure is measured via a noninvasive tail-cuff method for four days on the Visitech BP-2000 Blood Pressure Analysis System. The blood pressure is measured ten times each day for four days. The four days are then averaged to obtain a mouse's conscious systolic blood pressure.

Results:

The female (−/−) mice exhibited an increased mean systolic blood pressure when compared to its gender-matched (+/+) littermates (p=0.05) and the historical mean suggestive of hypertension in the homozygous mice.

(d) Phenotypic Analysis: Metabolism-Blood Chemistry/Glucose Tolerance

In the area of metabolism, targets may be identified for the treatment of diabetes. Blood chemistry phenotypic analysis includes blood glucose measurements. The COBAS Integra 400 (mfr: Roche) was used for running blood chemistry tests on the mice. In the area of metabolism, targets may be identified for the treatment of diabetes. Blood chemistry phenotypic analysis includes glucose tolerance tests to measure insulin sensitivity and changes in glucose metabolism. Abnormal glucose tolerance test results may indicate but may not be limited to the following disorders or conditions: Diabetes Type 1 and Type 2, Syndrome X, various cardiovascular diseases and/or obesity.

Procedure: A cohort of 2 wild type and 4 homozygous mice were used in this assay. The glucose tolerance test is the standard for defining impaired glucose homeostasis in mammals. Glucose tolerance tests were performed using a Lifescan glucometer. Animals were injected IP at 2 g/kg with D-glucose delivered as a 20% solution and blood glucose levels were measured at 0, 30, 60 and 90 minutes after injection.

Results:

Glucose Tolerance Test: The mutant (−/−) mice tested exhibited an impaired glucose tolerance at T-60 when compared with their gender-matched (+/+) littermates and the historical means.

These studies indicated that (−/−) mice exhibit a decreased or impaired glucose tolerance in the presence of normal fasting glucose when compared with their gender-matched (+/+) littermates and the historical means. Thus, knockout mutant mice exhibited the phenotypic pattern of an impaired glucose homeostasis, and therefore PRO1411 polypeptides (or agonists thereof) or its encoding gene would be useful in the treatment of conditions associated with an impaired glucose homeostasis and/or various cardiovascular diseases, including diabetes.

70.72. Generation and Analysis of Mice Comprising DNA71180-1655 (UNQ755) Gene Disruptions

In these knockout experiments, the gene encoding PRO1486 polypeptides (designated as DNA71180-1655) (UNQ755) was disrupted. The gene specific information for these studies is as follows: the mutated mouse gene corresponds to nucleotide reference: NM_{—}019820 Mus musculus cerebellin 3 precursor protein (Cbln3); protein reference: Q9JHG0 ACCESSION:Q9JHG0 NID: Mus musculus (Mouse). CBLN3; the human gene sequence reference: AY359070 Homo sapiens clone DNA71180 cerebellin (UNQ755); the human protein sequence corresponds to reference:. Q6UW01 ACCESSION: Q6UW01 NID: Homo sapiens (Human). Cerebellin. The mouse gene of interest is Cbln3 (cerebellin 3 precursor protein), ortholog of human “CBLN3” (Swiss-Prot accession Q6UW01). Aliases include precerebellin 3, UNQ755, and PRO1486. Cbln3 is a putative secreted protein of the precerebellin family expressed primarily in cerebellum and dorsal cochlear nucleus. The protein may function as a ligand or as a component of extracellular matrix. Cbln3 contains a signal peptide and a complement component Cl q domain and forms a heteromer with precerebellin family member cerebellin 1. The biological role of this protein is not known; however, it has been proposed to be a candidate gene for the mouse mutation agitans, which is characterized by Purkinje cell atrophy, retarded growth, generalized tremor, and ataxia (Pang et al, J Neurosci 20(17):6333-9 (2000)).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 19 | 35 | 17 | 71 |
| Expected | 17.75 | 35.5 | 17.75 | 71 |

Chi-Sq.=0.23 Significance=0.8913661 (hom/n)=0.26 Avg. Litter Size=6

### Mutation Information

Mutation Type: Homologous Recombination (standard)
Description: Coding exons 1 through 4 were targeted (NCBI accession NM_{—}019820.2).
1. Wild-type Expression Panel: Expression of the target gene was detected in all 13 adult tissue samples tested by RT-PCR, except lung, skeletal muscle, and bone.
2. QC Expression: Disruption of the target gene was confirmed by Southern hybridization analysis.

70.72.1. Phenotypic Analysis (for Disrupted Gene: DNA71180-1655 (UNQ755)

(a) Overall Phenotypic Summary:

Mutation of the gene encoding the ortholog of a human hypothetical secreted protein resulted in knockout (−/−) mice exhibiting an increased trabecular connectivity density and midshaft femur total area. Gene disruption was confirmed by Southern blot.

(b) Bone Metabolism & Body Diagnostics: Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured. The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Bone MicroCT Analysis:

Procedure: MicroCT was also used to get very sensitive measurements of BMD. One vertebra and 1 femur were taken from a cohort of 4 wild type and 8 homozygous mice. Measurements were taken of lumbar 5 vertebra trabecular bone volume, trabecular thickness, connectivity density and midshaft femur total bone area and cortical thickness. The μCT40 scans provided detailed information on bone mass and architecture. Multiple bones were placed into sample holders and scanned automatically. Instrument software was used to select regions of interest for analysis. Trabecular bone parameters were analyzed in the fifth lumbar vertebrae (LV5) at 16 micrometer resolution and cortical bone parameters were analyzed in the femur midshaft at a resolution of 20 micrometers.

Results:

MicroCT: The (−/−) homozygous mutants exhibited increased bone-related measurements with increased trabecular connectivity density and midshaft femur total area when compared with the (+/+) control littermates and the historical means.

In summary, the (−/−) mice exhibited increased trabecular connectivity density and femoral mid-shaft cross-sectional area when compared with their gender-matched (+/+) littermates. These results indicate that the knockout mutant phenotype is associated with such bone abnormalities as osteopetrosis. Osteopetrosis is a condition characterized by abnormal thickening and hardening of bone and abnormal fragility of the bones. As such, PRO1486 polypeptides or agonists thereof would be beneficial for the treatment of osteopetrosis or other osteo-related diseases. On the other hand, inhibitors or antagonists of PRO1486 polypeptides would be useful in bone healing.

70.73. Generation and Analysis of Mice Comprising DNA73727-1673 (UNQ771) Gene Disruptions

In these knockout experiments, the gene encoding PRO1565 polypeptides (designated as DNA73727-1673) (UNQ771) was disrupted. The gene specific information for these studies is as follows: the mutated mouse gene corresponds to nucleotide reference: NM_{—}022322 Mus musculus tenomodulin (Tnmd); protein reference: Q9EP64 ACCESSION: Q9EP64 NID: Mus musculus (Mouse). Tenomodulin (TeM) (mTeM) (Chondromodulin-I like protein) (ChM1L) (mChM1L) (Myodulin) (Tendin); the human gene sequence reference: NM_{—}022144 Homo sapiens tenomodulin (TNMD); the human protein sequence corresponds to reference: Q9H2S6 Tenomodulin (TeM) (hTeM) (Chondromodulin-I like protein) (ChM1L) (hChM1L) (Myodulin) (Tendin) (UNQ771/PRO1565) gi|5077276|gb|AAK83109.1|chondromodulin-IB [Homo sapiens] gi|12231527|gb|AAG49144.1|tenomodulin [Homo sapiens] gi|25392187|pir∥JC7597 chondromodulin-I like protein, ChM1L—human gi|12698293|dbj|BAB21756.1|ChM1L [Homo sapiens].

The mouse gene of interest is Tnmd (tenomodulin), ortholog of human TNMD. Aliases include ChM1L, tendin, 1110017101Rik, myodulin, TEM, BRICD4, CHM1-LIKE, myodulin protein, tenomodulin protein, and BRICHOS domain-containing 4.

TNMD is a type II plasma membrane protein of the chondromodulin-I family that likely functions as a ligand involved in inhibiting angiogenesis. The protein consists of a signal anchor and an antiangiogenic domain. TNMD is expressed in epimysium and tendon of skeletal muscle, in tendon of the extraocular muscle, and in sclerocornea and fiber cells of the eye. In the retina, TNMD is expressed in the ganglion layer and in inner nuclear layer cells and retinal pigment epithelial cells. TNMD is likely to play a role in inhibiting vascularization of certain types of tissue (Yamana et al, Biochem Biophys Res Commun 280(4):1101-6 (2001); Shukunami et al, Biochem Biophys Res Commun 280(5):1323-7 (2001); Brandau et al, Dev Dyn 221(1):72-80 (2001); Oshima et al, Ophthalmol Vis Sci 44(5):1814-23 (2003); Oshima et al, J Cell Sci 117(Pt 13):2731-44 2004)).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 17 | 19 | 46 | 82 |
| Expected | 20.5 | 41 | 20.5 | 82 |

Chi-Sq.=44.12 Significance=2.6270236E-10 (hom/n)=0.56 Avg. Litter Size=0
   This project is X-linked, hemizygotes have no notable phenotype.

### Summary of X-linked Gene Distribution by Sex and Genotype

(Only the agouti pups from the male chimeras are included.)

Summary of X-linked Gene Distributions for Sex by Genotype

| | | | |
|---|---|---|---|
| | Agouti F1 | | F1a |
| Progeny | (M chimera × wt) | Progeny | (F het × wt) |

| Sex | wt | het | Sex | wt | het | hemi |
|---|---|---|---|---|---|---|
| M | 4 | 0 | M | 17 | n/a | 23 |
| F | 1 | 16 | F | 24 | 21 | n/a |

### Mutation Information

Mutation Type: Homologous Recombination (standard)
Description: Coding exons 1 and 2 were targeted (NCBI accession NM_{—}022322.2).
1. Wild-type Expression Panel: Expression of the target gene was detected in brain, spinal cord, eye, thymus, skeletal muscle, bone, and adipose among the 13 adult tissue samples tested by RT-PCR.
2. QC Expression: Disruption of the target gene was confirmed by Southern hybridization analysis.

70.73.1. Phenotypic Analysis (for Disrupted Gene: DNA73727-1673 (UNQ771)

(a) Overall Phenotypic Summary:

Mutation of the gene encoding the ortholog of human tenomodulin (TNMD) resulted in an increased mean body length in the homozygous (−/−) mice compared to (+/+) siblings. Female (−/−) mice also exhibited increased serum potassium levels. Gene disruption was confirmed by Southern blot.

(b) Bone Metabolism & Body Diagnostics

(1) Tissue Mass & Lean Body Mass Measurements—Dexa

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in total tissue mass (TTM).

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI, i.e., whole body, vertebrae, and both femurs).

Body Measurements (Body Length & Weight):

Body Measurements: A measurement of body length and weight was performed at approximately 16 weeks of age.

Results:

The female (−/−) mice exhibited increased mean body length when compared with their gender-matched (+/+) littermates.

(c) Phenotypic Analysis: Metabolism-Blood Chemistry

In the area of metabolism, targets may be identified for the treatment of metabolic disorders. Blood chemistry phenotypic analysis includes blood glucose measurements. The COBAS Integra 400 (mfr: Roche) was used for running blood chemistry tests on the mice. In addition to measuring blood glucose levels the following blood chemistry tests are also routinely performed: Alkaline Phosphatase; Alanine Amino-Transferase; Albumin; Bilirubin; Phosphorous; Creatinine; BUN=Blood Urea Nitrogen; Calcium; Uric Acid; Sodium; Potassium; and Chloride.

Results:

The female (−/−) mice exhibited increased mean serum potassium levels compared with their (−/+) littermates. This observation suggests that homozygous mice have an altered electrolyte balance which could be the result of kidney dysfunction.

70.74. Generation and Analysis of Mice Comprising DNA89220-2608 (UNQ1924) Gene Disruptions

In these knockout experiments, the gene encoding PRO4399 polypeptides (designated as DNA89220-2608) (UNQ1924) was disrupted. The gene specific information for these studies is as follows: the mutated mouse gene corresponds to nucleotide reference: NM_{—}153157 Mus musculus olfactomedin 3 (Olfm3), transcript variant B; protein reference: Q8QZWO Noelin 3 precursor (Olfactomedin 3) (Optimedin); the human gene sequence reference: AY358722 Homo sapiens clone DNA89220 olfM3 (UNQ1924); the human protein sequence corresponds to reference: Q96PB7 ACCESSION:Q96PB7 NID: Homo sapiens (Human). NOELIN 3 PRECURSOR.

The mouse gene of interest is Olfm3 (olfactomedin 3), ortholog of human OLFM3. Aliases include B230206G02Rik, optimedin, NOE3, OPTIMEDIN, noelin 3, and olfactomedin related ER localized protein 3.

OLFM3 is a secreted protein expressed primarily in brain and retina that associates with myocilin (MYOC), a glaucoma candidate gene. Interaction with MYOC suggests that OLFM3 may also be a candidate gene for glaucoma (Torrado et al, Hum Mol Genet. 11(11):1291-301 (2002)).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 14 | 43 | 20 | 77 |
| Expected | 19.25 | 38.5 | 19.25 | 77 |

Chi-Sq.=6.49 Significance=0.03896857 (hom/n)=0.26 Avg. Litter Size=0

### Mutation Information

Mutation Type: Homologous Recombination (standard)
Description: Coding exon 4 was targeted (NCBI accession NM_{—}153157.1).
1. Wild-type Expression Panel: Expression of the target gene was detected in brain, spinal cord, eye, kidney, and liver among the 13 adult tissue samples tested by RT-PCR.
2. QC Expression: Disruption of the target gene was confirmed by Southern hybridization analysis.

70.74.1. Phenotypic Analysis (for Disrupted Gene: DNA89220-2608 (UNQ1924)

(a) Overall Phenotypic Summary:

Mutation of the gene encoding the ortholog of human olfactomedin 3 (OLFM3) resulted in the observation that female homozygous (−/−) mice exhibit increased total tissue mass and total body fat content. Gene disruption was confirmed by Southern blot.

(b) Bone Metabolism & Radiology Phenotypic Analysis

In the area of bone metabolism, targets were identified herein for the treatment of arthritis, osteoporosis, osteopenia and osteopetrosis as well as identifying targets that promote bone healing. Tests included:
DEXA for measurement of bone mineral density on femur and vertebra
MicroCT for very high resolution and very high sensitivity measurements of bone mineral density for both trabecular and cortical bone.

Dexa Analysis—Test Description:

Procedure: A cohort of 4 wild type, 4 heterozygous and 8 homozygous mice were tested in this assay. Dual Energy X-ray Absorptiometry (DEXA) has been used successfully to identify changes in bone. Anesthetized animals were examined and bone mineral content (BMC), BMC/LBM ratios, volumetric bone mineral density (vBMD), total body BMD, femur BMD and vertebra BMD were measured.

The mouse was anesthetized by intraperitoneal injection of Avertin (1.25% 2,2,2,-tribromoethanol, 20 ml/kg body weight), body length and weight were measured, and then the mouse was placed in a prone position on the platform of the PIXImus™ Densitometer (Lunar Inc.) for a DEXA scan. Using Lunar PIXImus software, the bone mineral density (BMD) and fat composition (% fat) and total tissue mass (TTM) were determined in the regions of interest (ROI) [i.e., whole body, vertebrae, and both femurs].

Results:

Female homozygous (−/−) mice exhibited increased total tissue mass and total fat content when compared with their gender-matched wildtype (+/+) littermates and the historical means.

These studies suggest that mutant (−/−) non-human transgenic animals exhibit a negative phenotype that is associated with obesity. Thus, PRO4399 polypeptides or agonists thereof are essential for normal growth and metabolic processes and especially would be important in the prevention and/or treatment of obesity.

70.75. Generation and Analysis of Mice Comprising DNA84142-2613 (UNQ1929) Gene Disruptions

In these knockout experiments, the gene encoding PRO4404 polypeptides (designated as DNA84142-2613) (UNQ1929) was disrupted. The gene specific information for these studies is as follows: the mutated mouse gene corresponds to nucleotide reference: NM_{—}001003947 Mus musculus cytochrome P450, family 4, subfamily x, polypeptide 1 (Cyp4×1); protein reference: Q6A152 ACCESSION:Q6A152 NID: Mus musculus (Mouse). Cytochrome P450; the human gene sequence reference: NM_{—}178033 Homo sapiens cytochrome P450, family 4, subfamily X, polypeptide 1 (CYP4×1); the human protein sequence corresponds to reference: Q8N118 ACCESSION: Q8N118 NID: Homo sapiens (Human). Cyto chrome P450 4X1 (EC 1.14.14.1) (CYPIVX1) (UNQ1929/PRO4404).

The mouse gene of interest is Cyp4x1 (cytochrome P450, family 4, subfamily x, polypeptide 1), ortholog of human CYP4x1. Aliases include CYP_a; A230025G20; cytochrome P450, 4x1; and MGC40051.

CYP4X1 is a putative heme-containing monooxygenase that likely catalyzes the oxidation of various molecules, such as steroids, fatty acids, bile acids, toxins, drugs, and other xenobiotics, with reduced flavoprotein and molecular oxygen as cosubstrates. The enzyme is expressed primarily in brain, including neurons and vascular endothelial cells, and is predicted to be located in the endoplasmic reticulum. The biological role of this enzyme is unknown; however, it may be involved in neurovascular function (Bylund et al, Biochem Biophys Res Commun 296(3):677-84 (2002)).

Targeted or gene trap mutations are generated in strain 129SvEv^{Brd}-derived embryonic stem (ES) cells. The chimeric mice are bred to C57BL/6J albino mice to generate F1 heterozygous animals. These progeny are intercrossed to generate F2 wild type, heterozygous, and homozygous mutant progeny. On rare occasions, for example when very few F1 mice are obtained from the chimera, F1 heterozygous mice are crossed to 129SvEv^{Brd}/C57 hybrid mice to yield additional heterozygous animals for the intercross to generate the F2 mice. Level I phenotypic analysis is performed on mice from this generation

| | wt | het | hom | Total |
|---|---|---|---|---|
| Observed | 13 | 33 | 20 | 66 |
| Expected | 16.5 | 33 | 16.5 | 66 |

Chi-Sq.=0.64 Significance=0.726149 (hom/n)=0.23 Avg. Litter Size=9

### Mutation Information

Mutation Type: Homologous Recombination (standard)
Description: Coding exon 1 was targeted (NCBI accession NM_{—}001003947.1).
1. Wild-type Expression Panel: Expression of the target gene was detected in embryonic stem (ES) cells and, among the 13 adult tissue samples tested by RT-PCR, in brain, spinal cord, eye, spleen, and kidney.
2. QC Expression: Disruption of the target gene was confirmed by Southern hybridization analysis.

70.75.1. Phenotypic Analysis (for Disrupted Gene: DNA84142-2613 (UNQ1929)

(a) Overall Phenotypic Summary:

Mutation of the gene encoding the ortholog of human cytochrome P450, family 4, subfamily x, polypeptide 1 (CYP4X1) resulted in the mutant (−/−) mice exhibiting a prolonged latency period during the hot plate testing. Gene disruption was confirmed by Southern blot.

(b) Phenotypic Analysis: CNS/Neurology

In the area of neurology, analysis focused herein on identifying in vivo validated targets for the treatment of neurological and psychiatric disorders including depression, generalized anxiety disorders, attention deficit hyperactivity disorder, obsessive compulsive disorder, schizophrenia, cognitive disorders, hyperalgesia and sensory disorders. Neurological disorders include the category defined as “anxiety disorders” which include but are not limited to: mild to moderate anxiety, anxiety disorder due to a general medical condition, anxiety disorder not otherwise specified, generalized anxiety disorder, panic attack, panic disorder with agoraphobia, panic disorder without agoraphobia, posttraumatic stress disorder, social phobia, specific phobia, substance-induced anxiety disorder, acute alcohol withdrawal, obsessive compulsive disorder, agoraphobia, bipolar disorder I or II, bipolar disorder not otherwise specified, cyclothymic disorder, depressive disorder, major depressive disorder, mood disorder, substance-induced mood disorder. In addition, anxiety disorders may apply to personality disorders including but not limited to the following types: paranoid, antisocial, avoidant behavior, borderline personality disorders, dependent, histronic, narcissistic, obsessive-compulsive, schizoid, and schizotypal.

Procedure:

Behavioral screens were performed on a cohort of 4 wild type, 4 heterozygous and 8 homozygous mice. All behavioral tests were done between 12 and 16 weeks of age unless reduced viability necessitates earlier testing.

Hot Plate Testing

Test Description: The hot plate test for nociception is carried out by placing each mouse on a small enclosed 55° C. hotplate. Latency to a hindlimb response (lick, shake, or jump) is recorded, with a maximum time on the hot plate of 30 sec. Each animal is tested once.

Results:

The female mutant (−/−) mice exhibited a prolonged latency to respond (for example a decreased sensitivity-difference) when compared with their gender-matched (+/+) littermate controls. These results suggest an alteration of pain perception.

### Example 71

### Use of PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 as a hybridization probe

The following method describes use of a nucleotide sequence encoding a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide as a hybridization probe.

DNA comprising the coding sequence of full-length or mature PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides as disclosed herein is employed as a probe to screen for homologous DNAs (such as those encoding naturally-occurring variants of PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides) inhuman tissue cDNA libraries or human tissue genomic libraries.

Hybridization and washing of filters containing either library DNAs is performed under the following high stringency conditions. Hybridization of radiolabeled PRO179-, PRO181-, PRO244-, PRO247-, PRO269-, PRO293-, PRO298-, PRO339-, PRO341-, PRO347-, PRO531-, PRO537-, PRO718-, PRO773-, PRO860-, PRO871-, PRO872-, PRO813-, PRO828-, PRO1100-, PRO1114-, PRO1115-, PRO1126-, PRO1133-, PRO1154-, PRO1185-, PRO1194-, PRO1287-, PRO1291-, PRO1293-, PRO1310-, PRO1312-, PRO1335-, PRO1339-, PRO2155-, PRO1356-, PRO1385-, PRO1412-, PRO1487-, PRO1758-, PRO1779-, PRO1785-, PRO1889-, PRO90318-, PRO3434-, PRO3579-, PRO4322-, PRO4343-, PRO4347-, PRO4403-, PRO4976-, PRO260-, PRO6014-, PRO6027-, PRO6181-, PRO6714-, PRO9922-, PRO7179-, PRO7476-, PRO9824-, PRO19814-, PRO19836-, PRO20088-, PRO70789-, PRO50298-, PRO51592-, PRO1757-, PRO4421-, PRO9903-, PRO1106-, PRO1411-, PRO1486-, PRO1565-, PRO4399- or PRO4404-derived probe to the filters is performed in a solution of 50% formamide, 5×SSC, 0.1% SDS, 0.1% sodium pyrophosphate, 50 mM sodium phosphate, pH 6.8, 2×Denhardt's solution, and 10% dextran sulfate at 42° C. for 20 hours. Washing of the filters is performed in an aqueous solution of 0.1×SSC and 0.1% SDS at 42° C.

DNAs having a desired sequence identity with the DNA encoding full-length native sequence PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides can then be identified using standard techniques known in the art.

### Example 72

### Expression of PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 in E. coli

This example illustrates preparation of an unglycosylated form of PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides by recombinant expression in E. coli.

The DNA sequence encoding a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide is initially amplified using selected PCR primers. The primers should contain restriction enzyme sites which correspond to the restriction enzyme sites on the selected expression vector. A variety of expression vectors may be employed. An example of a suitable vector is pBR322 (derived from E. coli; see Bolivar et al., Gene, 2:95 (1977)) which contains genes for ampicillin and tetracycline resistance. The vector is digested with restriction enzyme and dephosphorylated. The PCR amplified sequences are then ligated into the vector. The vector will preferably include sequences which encode for an antibiotic resistance gene, a tip promoter, a polyhis leader (including the first six STII codons, polyhis sequence, and enterokinase cleavage site), the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 coding region, lambda transcriptional terminator, and an argU gene.

The ligation mixture is then used to transform a selected E. coli strain using the methods described in Sambrook et al., supra. Transformants are identified by their ability to grow on LB plates and antibiotic resistant colonies are then selected. Plasmid DNA can be isolated and confirmed by restriction analysis and DNA sequencing.

Selected clones can be grown overnight in liquid culture medium such as LB broth supplemented with antibiotics. The overnight culture may subsequently be used to inoculate a larger scale culture. The cells are then grown to a desired optical density, during which the expression promoter is turned on.

After culturing the cells for several more hours, the cells can be harvested by centrifugation. The cell pellet obtained by the centrifugation can be solubilized using various agents known in the art, and the solubilized PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 protein can then be purified using a metal chelating column under conditions that allow tight binding of the protein.

PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 may be expressed in E. coli in a poly-His tagged form, using the following procedure. The DNA encoding PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 is initially amplified using selected PCR primers. The primers will contain restriction enzyme sites which correspond to the restriction enzyme sites on the selected expression vector, and other useful sequences providing for efficient and reliable translation initiation, rapid purification on a metal chelation column, and proteolytic removal with enterokinase. The PCR-amplified, poly-His tagged sequences are then ligated into an expression vector, which is used to transform an E. coli host based on strain 52 (W3110 fuhA(tonA) lon galE rpoHts(htpRts) clpP(lacIq). Transformants are first grown in LB containing 50 mg/ml carbenicillin at 30° C. with shaking until an O.D.600 of 3-5 is reached. Cultures are then diluted 50-100 fold into CRAP media (prepared by mixing 3.57 g (NH₄)₂SO₄, 0.71 g sodium citrate.2H2O, 1.07 g KCl, 5.36 g Difco yeast extract, 5.36 g Sheffield hycase SF in 500 mL water, as well as 110 mM MPOS, pH 7.3, 0.55% (w/v) glucose and 7 mM MgSO₄) and grown for approximately 20-30 hours at 30° C. with shaking. Samples are removed to verify expression by SDS-PAGE analysis, and the bulk culture is centrifuged to pellet the cells. Cell pellets are frozen until purification and refolding.

E. coli paste from 0.5 to 1 L fermentations (6-10 g pellets) is resuspended in 10 volumes (w/v) in 7 M guanidine, 20 mM Tris, pH 8 buffer. Solid sodium sulfite and sodium tetrathionate is added to make final concentrations of 0.1M and 0.02 M, respectively, and the solution is stirred overnight at 4° C. This step results in a denatured protein with all cysteine residues blocked by sulfitolization. The solution is centrifuged at 40,000 rpm in a Beckman Ultracentifuge for 30 min. The supernatant is diluted with 3-5 volumes of metal chelate column buffer (6 M guanidine, 20 mM Tris, pH 7.4) and filtered through 0.22 micron filters to clarify. The clarified extract is loaded onto a 5 ml Qiagen Ni-NTA metal chelate column equilibrated in the metal chelate column buffer. The column is washed with additional buffer containing 50 mM imidazole (Calbiochem, Utrol grade), pH 7.4. The protein is eluted with buffer containing 250 mM imidazole. Fractions containing the desired protein are pooled and stored at 4° C. Protein concentration is estimated by its absorbance at 280 nm using the calculated extinction coefficient based on its amino acid sequence.

The proteins are refolded by diluting the sample slowly into freshly prepared refolding buffer consisting of: 20 mM Tris, pH 8.6, 0.3 M NaCl, 2.5 M urea, 5 mM cysteine, 20 mM glycine and 1 mM EDTA. Refolding volumes are chosen so that the final protein concentration is between 50 to 100 micrograms/ml. The refolding solution is stirred gently at 4° C. for 12-36 hours. The refolding reaction is quenched by the addition of TFA to a final concentration of 0.4% (pH of approximately 3). Before further purification of the protein, the solution is filtered through a 0.22 micron filter and acetonitrile is added to 2-10% final concentration. The refolded protein is chromatographed on a Poros R1/H reversed phase column using a mobile buffer of 0.1% TFA with elution with a gradient of acetonitrile from 10 to 80%. Aliquots of fractions with A280 absorbance are analyzed on SDS polyacrylamide gels and fractions containing homogeneous refolded protein are pooled. Generally, the properly refolded species of most proteins are eluted at the lowest concentrations of acetonitrile since those species are the most compact with their hydrophobic interiors shielded from interaction with the reversed phase resin. Aggregated species are usually eluted at higher acetonitrile concentrations. In addition to resolving misfolded forms of proteins from the desired form, the reversed phase step also removes endotoxin from the samples.

Fractions containing the desired folded PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide are pooled and the acetonitrile removed using a gentle stream of nitrogen directed at the solution. Proteins are formulated into 20 mM Hepes, pH 6.8 with 0.14 M sodium chloride and 4% mannitol by dialysis or by gel filtration using G25 Superfine (Pharmacia) resins equilibrated in the formulation buffer and sterile filtered.

### Example 73

### Expression of PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 in mammalian cells

This example illustrates preparation of a potentially glycosylated form of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide by recombinant expression in mammalian cells.

The vector, pRK5 (see EP 307,247, published Mar. 15, 1989), is employed as the expression vector. Optionally, the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 DNA is ligated into pRK5 with selected restriction enzymes to allow insertion of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 DNA using ligation methods such as described in Sambrook et al., supra. The resulting vector is called pRK5-PRO179, pRK5-PRO181, pRK5-PRO244, pRK5-PRO247, pRK5-PRO269, pRK5-PRO293, pRK5-PRO298, pRK5-PRO339, pRK5-PRO341, pRK5-PRO347, pRK5-PRO531, pRK5-PRO537, pRK5-PRO718, pRK5-PRO773, pRK5-PRO860, pRK5-PRO871, pRK5-PRO872, pRK5-PRO813, pRK5-PRO828, pRK5-PRO1100, pRK5-PRO1114, pRK5-PRO1115, pRK5-PRO1126, pRK5-PRO1133, pRK5-PRO1154, pRK5-PRO1185, pRK5-PRO1194, pRK5-PRO1287, pRK5-PRO1291, pRK5-PRO1293, pRK5-PRO1310, pRK5-PRO1312, pRK5-PRO1335, pRK5-PRO1339, pRK5-PRO2155, pRK5-PRO1356, pRK5-PRO1385, pRK5-PRO1412, pRK5-PRO1487, pRK5-PRO1758, pRK5-PRO1779, pRK5-PRO1785, pRK5-PRO1889, pRK5-PRO90318, pRK5-PRO3434, pRK5-PRO3579, pRK5-PRO4322, pRK5-PRO4343, pRK5-PRO4347, pRK5-PRO4403, pRK5-PRO4976, pRK5-PRO260, pRK5-PRO6014, pRK5-PRO6027, pRK5-PRO6181, pRK5-PRO6714, pRK5-PRO9922, pRK5-PRO7179, pRK5-PRO7476, pRK5-PRO9824, pRK5-PRO19814, pRK5-PRO19836, pRK5-PRO20088, pRK5-PRO70789, pRK5-PRO50298, pRK5-PRO51592, pRK5-PRO1757, pRK5-PRO4421, pRK5-PRO9903, pRK5-PRO1106, pRK5-PRO1411, pRK5-PRO1486, pRK5-PRO1565, pRK5-PRO4399 or pRK5-PRO4404.

The selected host cells may be 293 cells. Human 293 cells (ATCC CCL 1573) are grown to confluence in tissue culture plates in medium such as DMEM supplemented with fetal calf serum and optionally, nutrient components and/or antibiotics. About 10 μg pRK5-PRO179, pRK5-PRO181, pRK5-PRO244, pRK5-PRO247, pRK5-PRO269, pRK5-PRO293, pRK5-PRO298, pRK5-PRO339, pRK5-PRO341, pRK5-PRO347, pRK5-PRO531, pRK5-PRO537, pRK5-PRO718, pRK5-PRO773, pRK5-PRO860, pRK5-PRO871, pRK5-PRO872, pRK5-PRO813, pRK5-PRO828, pRK5-PRO1100, pRK5-PRO1114, pRK5-PRO1115, pRK5-PRO1126, pRK5-PRO1133, pRK5-PRO1154, pRK5-PRO1185, pRK5-PRO1194, pRK5-PRO1287, pRK5-PRO1291, pRK5-PRO1293, pRK5-PRO1310, pRK5-PRO1312, pRK5-PRO1335, pRK5-PRO1339, pRK5-PRO2155, pRK5-PRO1356, pRK5-PRO1385, pRK5-PRO1412, pRK5-PRO1487, pRK5-PRO1758, pRK5-PRO1779, pRK5-PRO1785, pRK5-PRO1889, pRK5-PRO90318, pRK5-PRO3434, pRK5-PRO3579, pRK5-PRO4322, pRK5-PRO4343, pRK5-PRO4347, pRK5-PRO4403, pRK5-PRO4976, pRK5-PRO260, pRK5-PRO6014, pRK5-PRO6027, pRK5-PRO6181, pRK5-PRO6714, pRK5-PRO9922, pRK5-PRO7179, pRK5-PRO7476, pRK5-PRO9824, pRK5-PRO19814, pRK5-PRO19836, pRK5-PRO20088, pRK5-PRO70789, pRK5-PRO50298, pRK5-PRO51592, pRK5-PRO1757, pRK5-PRO4421, pRK5-PRO9903, pRK5-PRO1106, pRK5-PRO1411, pRK5-PRO1486, pRK5-PRO1565, pRK5-PRO4399 or pRK5-PRO4404 DNA is mixed with about 1 μg DNA encoding the VA RNA gene [Thimmappaya et al., Cell, 31:543 (1982)] and dissolved in 500 μl of 1 mM Tris-HCl, 0.1 mM EDTA, 0.227 M CaCl₂. To this mixture is added, dropwise, 500 μl of 50 mM HEPES (pH 7.35), 280 mM NaCl, 1.5 mM NaPO₄, and a precipitate is allowed to form for 10 minutes at 25° C. The precipitate is suspended and added to the 293 cells and allowed to settle for about four hours at 37° C. The culture medium is aspirated off and 2 ml of 20% glycerol in PBS is added for 30 seconds. The 293 cells are then washed with serum free medium, fresh medium is added and the cells are incubated for about 5 days.

Approximately 24 hours after the transfections, the culture medium is removed and replaced with culture medium (alone) or culture medium containing 200 μCi/ml ³⁵S-cysteine and 200 μCi/ml ³⁵S-methionine. After a 12 hour incubation, the conditioned medium is collected, concentrated on a spin filter, and loaded onto a 15% SDS gel. The processed gel may be dried and exposed to film for a selected period of time to reveal the presence of PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides. The cultures containing transfected cells may undergo further incubation (in serum free medium) and the medium is tested in selected bioassays.

In an alternative technique, PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 may be introduced into 293 cells transiently using the dextran sulfate method described by Somparyrac et al., Proc. Natl. Acad. Sci., 12:7575 (1981). 293 cells are grown to maximal density in a spinner flask and 700 μg pRK5-PRO179, pRK5-PRO181, pRK5-PRO244, pRK5-PRO247, pRK5-PRO269, pRK5-PRO293, pRK5-PRO298, pRK5-PRO339, pRK5-PRO341, pRK5-PRO347, pRK5-PRO531, pRK5-PRO537, pRK5-PRO718, pRK5-PRO773, pRK5-PRO860, pRK5-PRO871, pRK5-PRO872, pRK5-PRO813, pRK5-PRO828, pRK5-PRO1100, pRK5-PRO1114, pRK5-PRO1115, pRK5-PRO1126, pRK5-PRO1133, pRK5-PRO1154, pRK5-PRO1185, pRK5-PRO1194, pRK5-PRO1287, pRK5-PRO1291, pRK5-PRO1293, pRK5-PRO1310, pRK5-PRO1312, pRK5-PRO1335, pRK5-PRO1339, pRK5-PRO2155, pRK5-PRO1356, pRK5-PRO1385, pRK5-PRO1412, pRK5-PRO1487, pRK5-PRO1758, pRK5-PRO1779, pRK5-PRO1785, pRK5-PRO1889, pRK5-PRO90318, pRK5-PRO3434, pRK5-PRO3579, pRK5-PRO4322, pRK5-PRO4343, pRK5-PRO4347, pRK5-PRO4403, pRK5-PRO4976, pRK5-PRO260, pRK5-PRO6014, pRK5-PRO6027, pRK5-PRO6181, pRK5-PRO6714, pRK5-PRO9922, pRK5-PRO7179, pRK5-PRO7476, pRK5-PRO9824, pRK5-PRO19814, pRK5-PRO19836, pRK5-PRO20088, pRK5-PRO70789, pRK5-PRO50298, pRK5-PRO51592, pRK5-PRO1757, pRK5-PRO4421, pRK5-PRO9903, pRK5-PRO1106, pRK5-PRO1411, pRK5-PRO1486, pRK5-PRO1565, pRK5-PRO4399 or pRK5-PRO4404 DNA is added. The cells are first concentrated from the spinner flask by centrifugation and washed with PBS. The DNA-dextran precipitate is incubated on the cell pellet for four hours. The cells are treated with 20% glycerol for 90 seconds, washed with tissue culture medium, and re-introduced into the spinner flask containing tissue culture medium, 5 μg/ml bovine insulin and 0.1 μg/ml bovine transferrin. After about four days, the conditioned media is centrifuged and filtered to remove cells and debris. The sample containing expressed PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 can then be concentrated and purified by any selected method, such as dialysis and/or column chromatography.

PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 can be expressed in CHO cells. The pRK5-PRO179, pRK5-PRO181, pRK5-PRO244, pRK5-PRO247, pRK5-PRO269, pRK5-PRO293, pRK5-PRO298, pRK5-PRO339, pRK5-PRO341, pRK5-PRO347, pRK5-PRO531, pRK5-PRO537, pRK5-PRO718, pRK5-PRO773, pRK5-PRO860, pRK5-PRO871, pRK5-PRO872, pRK5-PRO813, pRK5-PRO828, pRK5-PRO1100, pRK5-PRO1114, pRK5-PRO1115, pRK5-PRO1126, pRK5-PRO1133, pRK5-PRO1154, pRK5-PRO1185, pRK5-PRO1194, pRK5-PRO1287, pRK5-PRO1291, pRK5-PRO1293, pRK5-PRO1310, pRK5-PRO1312, pRK5-PRO1335, pRK5-PRO1339, pRK5-PRO2155, pRK5-PRO1356, pRK5-PRO1385, pRK5-PRO1412, pRK5-PRO1487, pRK5-PRO1758, pRK5-PRO1779, pRK5-PRO1785, pRK5-PRO1889, pRK5-PRO90318, pRK5-PRO3434, pRK5-PRO3579, pRK5-PRO4322, pRK5-PRO4343, pRK5-PRO4347, pRK5-PRO4403, pRK5-PRO4976, pRK5-PRO260, pRK5-PRO6014, pRK5-PRO6027, pRK5-PRO6181, pRK5-PRO6714, pRK5-PRO9922, pRK5-PRO7179, pRK5-PRO7476, pRK5-PRO9824, pRK5-PRO19814, pRK5-PRO19836, pRK5-PRO20088, pRK5-PRO70789, pRK5-PRO50298, pRK5-PRO51592, pRK5-PRO1757, pRK5-PRO4421, pRK5-PRO9903, pRK5-PRO1106, pRK5-PRO1411, pRK5-PRO1486, pRK5-PRO1565, pRK5-PRO4399 or pRK5-PRO4404 can be transfected into CHO cells using known reagents such as CaPO₄ or DEAE-dextran. As described above, the cell cultures can be incubated, and the medium replaced with culture medium (alone) or medium containing a radiolabel such as ³⁵S-methionine. After determining the presence of PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the culture medium may be replaced with serum free medium. Preferably, the cultures are incubated for about 6 days, and then the conditioned medium is harvested. The medium containing the expressed PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 can then be concentrated and purified by any selected method.

Epitope-tagged PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 may also be expressed in host CHO cells. The PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 may be subcloned out of the pRK5 vector. The subclone insert can undergo PCR to fuse in frame with a selected epitope tag such as a poly-his tag into a Baculovirus expression vector. The poly-his tagged PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 insert can then be subcloned into a SV40 driven vector containing a selection marker such as DHFR for selection of stable clones. Finally, the CHO cells can be transfected (as described above) with the SV40 driven vector. Labeling may be performed, as described above, to verify expression. The culture medium containing the expressed poly-His tagged PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 can then be concentrated and purified by any selected method, such as by Ni²⁺-chelate affinity chromatography.

PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 may also be expressed in CHO and/or COS cells by a transient expression procedure or in CHO cells by another stable expression procedure.

Stable expression in CHO cells is performed using the following procedure. The proteins are expressed as an IgG construct (immunoadhesin), in which the coding sequences for the soluble forms (e.g. extracellular domains) of the respective proteins are fused to an IgG1 constant region sequence containing the hinge, CH2 and CH2 domains and/or is a poly-His tagged form.

Following PCR amplification, the respective DNAs are subcloned in a CHO expression vector using standard techniques as described in Ausubel et al., Current Protocols of Molecular Biology, Unit 3.16, John Wiley and Sons (1997). CHO expression vectors are constructed to have compatible restriction sites 5′ and 3′ of the DNA of interest to allow the convenient shuttling of cDNA's. The vector used expression in CHO cells is as described in Lucas et al., Nucl. Acids Res. 24:9 (1774-1779 (1996), and uses the SV40 early promoter/enhancer to drive expression of the cDNA of interest and dihydrofolate reductase (DHFR). DHFR expression permits selection for stable maintenance of the plasmid following transfection.

Twelve micrograms of the desired plasmid DNA is introduced into approximately 10 million CHO cells using commercially available transfection reagents Superfect® (Qiagen), Dosper® or Fugene® (Boehringer Mannheim). The cells are grown as described in Lucas et al., supra. Approximately 3×10⁷ cells are frozen in an ampule for further growth and production as described below.

The ampules containing the plasmid DNA are thawed by placement into water bath and mixed by vortexing. The contents are pipetted into a centrifuge tube containing 10 mLs of media and centrifuged at 1000 rpm for 5 minutes. The supernatant is aspirated and the cells are resuspended in 10 mL of selective media (0.2 filtered PS20 with 5% 0.2 μm diafiltered fetal bovine serum). The cells are then aliquoted into a 100 mL spinner containing 90 mL of selective media. After 1-2 days, the cells are transferred into a 250 mL spinner filled with 150 mL selective growth medium and incubated at 37° C. After another 2-3 days, 250 mL, 500 mL and 2000 mL spinners are seeded with 3×10⁵ cells/mL. The cell media is exchanged with fresh media by centrifugation and resuspension in production medium. Although any suitable CHO media may be employed, a production medium described in U.S. Pat. No. 5,122,469, issued Jun. 16, 1992 may actually be used. A 3 L production spinner is seeded at 1.2×10⁶ cells/mL. On day 0, the cell number pH ie determined. On day 1, the spinner is sampled and sparging with filtered air is commenced. On day 2, the spinner is sampled, the temperature shifted to 33° C., and 30 mL of 500 g/L glucose and 0.6 mL of 10% antifoam (e.g., 35% polydimethylsiloxane emulsion, Dow Corning 365 Medical Grade Emulsion) taken. Throughout the production, the pH is adjusted as necessary to keep it at around 7.2. After 10 days, or until the viability dropped below 70%, the cell culture is harvested by centrifugation and filtering through a 0.22 μm filter. The filtrate was either stored at 4° C. or immediately loaded onto columns for purification.

For the poly-His tagged constructs, the proteins are purified using a Ni-NTA column (Qiagen). Before purification, imidazole is added to the conditioned media to a concentration of 5 mM. The conditioned media is pumped onto a 6 ml Ni-NTA column equilibrated in 20 mM Hepes, pH 7.4, buffer containing 0.3 M NaCl and 5 mM imidazole at a flow rate of 4-5 ml/min at 4° C. After loading, the column is washed with additional equilibration buffer and the protein eluted with equilibration buffer containing 0.25 M imidazole. The highly purified protein is subsequently desalted into a storage buffer containing 10 mM Hepes, 0.14 M NaCl and 4% mannitol, pH 6.8, with a 25 ml G25 Superfine (Pharmacia) column and stored at −80° C.

Immunoadhesin (Fe-containing) constructs are purified from the conditioned media as follows. The conditioned medium is pumped onto a 5 ml Protein A column (Pharmacia) which had been equilibrated in 20 mM Na phosphate buffer, pH 6.8. After loading, the column is washed extensively with equilibration buffer before elution with 100 mM citric acid, pH 3.5. The eluted protein is immediately neutralized by collecting 1 ml fractions into tubes containing 275 μL of 1 M Tris buffer, pH 9. The highly purified protein is subsequently desalted into storage buffer as described above for the poly-His tagged proteins. The homogeneity is assessed by SDS polyacrylamide gels and by N-terminal amino acid sequencing by Edman degradation.

### Example 74

### Expression of PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 in Yeast

The following method describes recombinant expression of PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 in yeast.

First, yeast expression vectors are constructed for intracellular production or secretion of PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 from the ADH2/GAPDH promoter. DNA encoding PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 and the promoter is inserted into suitable restriction enzyme sites in the selected plasmid to direct intracellular expression of PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404. For secretion, DNA encoding PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 can be cloned into the selected plasmid, together with DNA encoding the ADH2/GAPDH promoter, a native PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 signal peptide or other mammalian signal peptide, or, for example, a yeast alpha-factor or invertase secretory signal/leader sequence, and linker sequences (if needed) for expression of PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404.

Yeast cells, such as yeast strain AB110, can then be transformed with the expression plasmids described above and cultured in selected fermentation media. The transformed yeast supernatants can be analyzed by precipitation with 10% trichloroacetic acid and separation by SDS-PAGE, followed by staining of the gels with Coomassie Blue stain.

Recombinant PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 can subsequently be isolated and purified by removing the yeast cells from the fermentation medium by centrifugation and then concentrating the medium using selected cartridge filters. The concentrate containing PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 may further be purified using selected column chromatography resins.

### Example 75

### Expression of PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 in Baculovirus-Infected Insect Cells

The following method describes recombinant expression of PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 in Baculovirus-infected insect cells.

The sequence coding for PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 is fused upstream of an epitope tag contained within a baculovirus expression vector. Such epitope tags include poly-his tags and immunoglobulin tags (like Fc regions of IgG). A variety of plasmids may be employed, including plasmids derived from commercially available plasmids such as pVL1393 (Novagen). Briefly, the sequence encoding PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 or the desired portion of the coding sequence of PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 such as the sequence encoding the extracellular domain of a transmembrane protein or the sequence encoding the mature protein if the protein is extracellular is amplified by PCR with primers complementary to the 5′ and 3′ regions. The 5′ primer may incorporate flanking (selected) restriction enzyme sites. The product is then digested with those selected restriction enzymes and subcloned into the expression vector.

Recombinant baculovirus is generated by co-transfecting the above plasmid and BaculoGold™ virus DNA (Pharmingen) into Spodoptera frugiperda (“Sf9”) cells (ATCC CRL 1711) using lipofectin (commercially available from GIBCO-BRL). After 4-5 days of incubation at 28° C., the released viruses are harvested and used for further amplifications. Viral infection and protein expression are performed as described by O'Reilley et al., Baculovirus expression vectors: A Laboratory Manual, Oxford: Oxford University Press (1994).

Expressed poly-his tagged PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 can then be purified, for example, by Ni²⁺-chelate affinity chromatography as follows. Extracts are prepared from recombinant virus-infected Sf9 cells as described by Rupert et al., Nature, 362:175-179 (1993). Briefly, Sf9 cells are washed, resuspended in sonication buffer (25 mL Hepes, pH 7.9; 12.5 mM MgCl₂; 0.1 mM EDTA; 10% glycerol; 0.1% NP-40; 0.4 M KCl), and sonicated twice for 20 seconds on ice. The sonicates are cleared by centrifugation, and the supernatant is diluted 50-fold in loading buffer (50 mM phosphate, 300 mM NaCl, 10% glycerol, pH 7.8) and filtered through a 0.45 μm filter. A Ni²⁺-NTA agarose column (commercially available from Qiagen) is prepared with a bed volume of 5 mL, washed with 25 mL of water and equilibrated with 25 mL of loading buffer. The filtered cell extract is loaded onto the column at 0.5 mL per minute. The column is washed to baseline A₂₈₀ with loading buffer, at which point fraction collection is started. Next, the column is washed with a secondary wash buffer (50 mM phosphate; 300 mMNaCl, 10% glycerol, pH 6.0), which elutes nonspecific ally bound protein. After reaching A₂₈₀ baseline again, the column is developed with a 0 to 500 mM Imidazole gradient in the secondary wash buffer. One mL fractions are collected and analyzed by SDS-PAGE and silver staining or Western blot with Ni²⁺-NTA-conjugated to alkaline phosphatase (Qiagen). Fractions containing the eluted His₁₀-tagged PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 are pooled and dialyzed against loading buffer.

Alternatively, purification of the IgG tagged (or Fc tagged) PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 can be performed using known chromatography techniques, including for instance, Protein A or protein G column chromatography.

### Example 76

### Tissue Expression Profiling Using GeneExpress®

A proprietary database containing gene expression information (GeneExpress®, Gene Logic Inc., Gaithersburg, Md.) was analyzed in an attempt to identify polypeptides (and their encoding nucleic acids) whose expression is significantly upregulated in a particular tumor tissue(s) of interest as compared to other tumor(s) and/or normal tissues. Specifically, analysis of the GeneExpress® database was conducted using either software available through Gene Logic Inc., Gaithersburg, Md., for use with the GeneExpress® database or with proprietary software written and developed at Genentech, Inc. for use with the GeneExpress® database. The rating of positive hits in the analysis is based upon several criteria including, for example, tissue specificity, tumor specificity and expression level in normal essential and/or normal proliferating tissues. The following is a list of molecules whose tissue expression profile as determined from an analysis of the GeneExpress® database evidences high tissue expression and significant upregulation of expression in a specific tumor or tumors as compared to other tumor(s) and/or normal tissues and optionally relatively low expression in normal essential and/or normal proliferating tissues.

### Example 77

### Microarray Analysis to Detect Upregulation of UNQ Genes in Cancerous Tumors

Nucleic acid microarrays, often containing thousands of gene sequences, are useful for identifying differentially expressed genes in diseased tissues as compared to their normal counterparts. Using nucleic acid microarrays, test and control mRNA samples from test and control tissue samples are reverse transcribed and labeled to generate cDNA probes. The cDNA probes are then hybridized to an array of nucleic acids immobilized on a solid support. The array is configured such that the sequence and position of each member of the array is known. For example, a selection of genes known to be expressed in certain disease states may be arrayed on a solid support. Hybridization of a labeled probe with a particular array member indicates that the sample from which the probe was derived expresses that gene. If the hybridization signal of a probe from a test (disease tissue) sample is greater than hybridization signal of a probe from a control (normal tissue) sample, the gene or genes overexpressed in the disease tissue are identified. The implication of this result is that an overexpressed protein in a diseased tissue is useful not only as a diagnostic marker for the presence of the disease condition, but also as a therapeutic target for treatment of the disease condition.

The methodology of hybridization of nucleic acids and microarray technology is well known in the art. In one example, the specific preparation of nucleic acids for hybridization and probes, slides, and hybridization conditions are all detailed in PCT Patent Application Serial No. PCT/US01/10482, filed on Mar. 30, 2001 and which is herein incorporated by reference.

In the present example, cancerous tumors derived from various human tissues were studied for upregulated gene expression relative to cancerous tumors from different tissue types and/or non-cancerous human tissues in an attempt to identify those polypeptides which are overexpressed in a particular cancerous tumor(s). In certain experiments, cancerous human tumor tissue and non-cancerous human tumor tissue of the same tissue type (often from the same patient) were obtained and analyzed for UNQ polypeptide expression. Additionally, cancerous human tumor tissue from any of a variety of different human tumors was obtained and compared to a “universal” epithelial control sample which was prepared by pooling non-cancerous human tissues of epithelial origin, including liver, kidney, and lung. mRNA isolated from the pooled tissues represents a mixture of expressed gene products from these different tissues. Microarray hybridization experiments using the pooled control samples generated a linear plot in a 2-color analysis. The slope of the line generated in a 2-color analysis was then used to normalize the ratios of (test:control detection) within each experiment. The normalized ratios from various experiments were then compared and used to identify clustering of gene expression. Thus, the pooled “universal control” sample not only allowed effective relative gene expression determinations in a simple 2-sample comparison, it also allowed multi-sample comparisons across several experiments.

In the present experiments, nucleic acid probes derived from the herein described UNQ polypeptide-encoding nucleic acid sequences were used in the creation of the microarray and RNA from various tumor tissues were used for the hybridization thereto. Below is shown the results of these experiments, demonstrating that various UNQ polypeptides of the present invention are significantly overexpressed in various human tumor tissues as compared to their normal counterpart tissue(s). Moreover, all of the molecules shown below are significantly overexpressed in their specific tumor tissue(s) as compared to in the “universal” epithelial control. As described above, these data demonstrate that the UNQ polypeptides of the present invention are useful not only as diagnostic markers for the presence of one or more cancerous tumors, but also serve as therapeutic targets for the treatment of those tumors.

### Example 78

### Quantitative Analysis of UNQ mRNA Expression

In this assay, a 5′ nuclease assay (for example, TagMan®) and real-time quantitative PCR (for example, ABI Prizm 7700 Sequence Detection System® (Perkin Elmer, Applied Biosystems Division, Foster City, Calif.)), were used to find genes that are significantly overexpressed in a cancerous tumor or tumors as compared to other cancerous tumors or normal non-cancerous tissue. The 5′ nuclease assay reaction is a fluorescent PCR-based technique which makes use of the 5′ exonuclease activity of Taq DNA polymerase enzyme to monitor gene expression in real time. Two oligonucleotide primers (whose sequences are based upon the gene or EST sequence of interest) are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the PCR amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

The 5′ nuclease procedure is run on a real-time quantitative PCR device such as the ABI Prism 7700™ Sequence Detection. The system consists of a thermocycler, laser, charge-coupled device (CCD) camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

The starting material for the screen was mRNA isolated from a variety of different cancerous tissues. The mRNA is quantitated precisely, e.g., fluorometrically. As a negative control, RNA was isolated from various normal tissues of the same tissue type as the cancerous tissues being tested.

5′ nuclease assay data are initially expressed as Ct, or the threshold cycle. This is defined as the cycle at which the reporter signal accumulates above the background level of fluorescence. The ΔCt values are used as quantitative measurement of the relative number of starting copies of a particular target sequence in a nucleic acid sample when comparing cancer mRNA results to normal human mRNA results. As one Ct unit corresponds to 1 PCR cycle or approximately a 2-fold relative increase relative to normal, two units corresponds to a 4-fold relative increase, 3 units corresponds to an 8-fold relative increase and so on, one can quantitatively measure the relative fold increase in mRNA expression between two or more different tissues. Using this technique, the molecules have been identified as being significantly overexpressed in a particular tumor(s) as compared to their normal non-cancerous counterpart tissue(s) (from both the same and different tissue donors) and thus, represent excellent polypeptide targets for the diagnosis and therapy of cancer in mammals.

### Example 79

### In Situ Hybridization

In situ hybridization is a powerful and versatile technique for the detection and localization of nucleic acid sequences within cell or tissue preparations. It may be useful, for example, to identify sites of gene expression, analyze the tissue distribution of transcription, identify and localize viral infection, follow changes in specific mRNA synthesis and aid in chromosome mapping.

In situ hybridization was performed following an optimized version of the protocol by Lu and Gillett, Cell Vision 1:169-176 (1994), using PCR-generated ³³P-labeled riboprobes. Briefly, formalin-fixed, paraffin-embedded human tissues were sectioned, deparaffinized, deproteinated in proteinase K (20 g/ml) for 15 minutes at 37° C., and further processed for in situ hybridization as described by Lu and Gillett, supra. A [³³-P] UTP-labeled antisense riboprobe was generated from a PCR product and hybridized at 55° C. overnight. The slides were dipped in Kodak NTB2 nuclear track emulsion and exposed for 4 weeks.

### ³³P-Riboprobe Synthesis

6.0 μl (125 mCi) of ³³P-UTP (Amersham BF 1002, SA<2000 Ci/mmol) were speed vac dried. To each tube containing dried ³³P-UTP, the following ingredients were added:

2.0 μl 5× transcription buffer

1.0 μl DTT (100 mM)

2.0 μl NTP mix (2.5 mM: 10 μl; each of 10 mM GTP, CTP & ATP+10 μl H₂O)

1.0 μl UTP (50 μM)

1.0 μl Rnasin

1.0 μl DNA template (1 μg)

1.0 μl H₂O

1.0 μl RNA polymerase (for PCR products T3=AS, T7=S, usually)

The tubes were incubated at 37° C. for one hour. 1.0 μl RQ1 DNase were added, followed by incubation at 37° C. for 15 minutes. 90 μl TE (10 mM Tris pH 7.6/1 mM EDTA pH 8.0) were added, and the mixture was pipetted onto DE81 paper. The remaining solution was loaded in a Microcon-50 ultrafiltration unit, and spun using program 10 (6 minutes). The filtration unit was inverted over a second tube and spun using program 2 (3 minutes). After the final recovery spin, 100 μl TE were added. 1 μl of the final product was pipetted on DE81 paper and counted in 6 ml of Biofluor II.

The probe was run on a TBE/urea gel. 1-3 μl of the probe or 5 μl of RNA Mrk III were added to 3 μl of loading buffer. After heating on a 95° C. heat block for three minutes, the probe was immediately placed on ice. The wells of gel were flushed, the sample loaded, and run at 180-250 volts for 45 minutes. The gel was wrapped in saran wrap and exposed to XAR film with an intensifying screen in −70° C. freezer one hour to overnight.

### ³³P-Hybridization

A. Pretreatment of Frozen Sections

The slides were removed from the freezer, placed on aluminium trays and thawed at room temperature for 5 minutes. The trays were placed in 55° C. incubator for five minutes to reduce condensation. The slides were fixed for 10 minutes in 4% paraformaldehyde on ice in the fume hood, and washed in 0.5×SSC for 5 minutes, at room temperature (25 ml 20×SSC+975 ml SQ H₂O). After deproteination in 0.5 μg/ml proteinase K for 10 minutes at 37° C. (12.5 μl of 10 mg/ml stock in 250 ml prewarmed RNase-free RNAse buffer), the sections were washed in 0.5×SSC for 10 minutes at room temperature. The sections were dehydrated in 70%, 95%, 100% ethanol, 2 minutes each.

B. Pretreatment of Paraffin-embedded Sections

The slides were deparaffinized, placed in SQ H₂O, and rinsed twice in 2×SSC at room temperature, for 5 minutes each time. The sections were deproteinated in 20 μg/ml proteinase K (500 μl of 10 mg/ml in 250 ml RNase-free RNase buffer; 37° C., 15 minutes)-human embryo, or 8× proteinase K (100 μl in 250 ml Rnase buffer, 37° C., 30 minutes)—formalin tissues. Subsequent rinsing in 0.5×SSC and dehydration were performed as described above.

C. Prehybridization

The slides were laid out in a plastic box lined with Box buffer (4×SSC, 50% formamide)—saturated filter paper.

D. Hybridization

1.0×10⁶ cpm probe and 1.0 μl tRNA (50 mg/ml stock) per slide were heated at 95° C. for 3 minutes. The slides were cooled on ice, and 48 μl hybridization buffer were added per slide. After vortexing, 50 μl ³³P mix were added to 50 μl prehybridization on slide. The slides were incubated overnight at 55° C.

E. Washes

Washing was done 2×10 minutes with 2×SSC, EDTA at room temperature (400 ml 20×SSC+16 ml 0.25M EDTA, V_{f}=4 L), followed by RNaseA treatment at 37° C. for 30 minutes (500 μl of 10 mg/ml in 250 ml Rnase buffer=20 μg/ml). The slides were washed 2×10 minutes with 2×SSC, EDTA at room temperature. The stringency wash conditions were as follows: 2 hours at 55° C., 0.1×SSC, EDTA (20 ml 20×SSC+16 ml EDTA, V_{f}=4L).

F. Oligonucleotides

In situ analysis was performed on a variety of DNA sequences disclosed herein. The oligonucleotides employed for these analyses were obtained so as to be complementary to the nucleic acids (or the complements thereof) as shown in the accompanying figures.

### Example 80

### Preparation of Antibodies that Bind PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404

This example illustrates preparation of monoclonal antibodies which can specifically bind PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404.

Techniques for producing the monoclonal antibodies are known in the art and are described, for instance, in Goding, supra. Immunogens that may be employed include purified PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides, fusion proteins containing PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides, and cells expressing recombinant PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides on the cell surface. Selection of the immunogen can be made by the skilled artisan without undue experimentation.

Mice, such as Balb/c, are immunized with the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 immunogen emulsified in complete Freund's adjuvant and injected subcutaneously or intraperitoneally in an amount from 1-100 micrograms. Alternatively, the immunogen is emulsified in MPL-TDM adjuvant (Ribi Immunochemical Research, Hamilton, Mont.) and injected into the animal's hind foot pads. The immunized mice are then boosted 10 to 12 days later with additional immunogen emulsified in the selected adjuvant. Thereafter, for several weeks, the mice may also be boosted with additional immunization injections. Serum samples may be periodically obtained from the mice by retro-orbital bleeding for testing in ELISA assays to detect anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibodies.

After a suitable antibody titer has been detected, the animals “positive” for antibodies can be injected with a final intravenous injection of PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404. Three to four days later, the mice are sacrificed and the spleen cells are harvested. The spleen cells are then fused (using 35% polyethylene glycol) to a selected murine myeloma cell line such as P3X63AgU.1, available from ATCC, No. CRL 1597. The fusions generate hybridoma cells which can then be plated in 96 well tissue culture plates containing HAT (hypoxanthine, aminopterin, and thymidine) medium to inhibit proliferation of non-fused cells, myeloma hybrids, and spleen cell hybrids.

The hybridoma cells will be screened in an ELISA for reactivity against PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404. Determination of “positive” hybridoma cells secreting the desired monoclonal antibodies against PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 is within the skill in the art.

The positive hybridoma cells can be injected intraperitoneally into syngeneic Balb/c mice to produce ascites containing the anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 monoclonal antibodies. Alternatively, the hybridoma cells can be grown in tissue culture flasks or roller bottles. Purification of the monoclonal antibodies produced in the ascites can be accomplished using ammonium sulfate precipitation, followed by gel exclusion chromatography. Alternatively, affinity chromatography based upon binding of antibody to protein A or protein G can be employed.

### Example 81

### Purification of PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 Polypeptides Using Specific Antibodies

Native or recombinant PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides may be purified by a variety of standard techniques in the art of protein purification. For example, pro-PRO179, pro-PRO181, pro-PRO244, pro-PRO247, pro-PRO269, pro-PRO293, pro-PRO298, pro-PRO339, pro-PRO341, pro-PRO347, pro-PRO531, pro-PRO537, pro-PRO718, pro-PRO773, pro-PRO860, pro-PRO871, pro-PRO872, pro-PRO813, pro-PRO828, pro-PRO1100, pro-PRO1114, pro-PRO1115, pro-PRO1126, pro-PRO1133, pro-PRO1154, pro-PRO1185, pro-PRO1194, pro-PRO1287, pro-PRO1291, pro-PRO1293, pro-PRO1310, pro-PRO1312, pro-PRO1335, pro-PRO1339, pro-PRO2155, pro-PRO1356, pro-PRO1385, pro-PRO1412, pro-PRO1487, pro-PRO1758, pro-PRO1779, pro-PRO1785, pro-PRO1889, pro-PRO90318, pro-PRO3434, pro-PRO3579, pro-PRO4322, pro-PRO4343, pro-PRO4347, pro-PRO4403, pro-PRO4976, pro-PRO260, pro-PRO6014, pro-PRO6027, pro-PRO6181, pro-PRO6714, pro-PRO9922, pro-PRO7179, pro-PRO7476, pro-PRO9824, pro-PRO19814, pro-PRO19836, pro-PRO20088, pro-PRO70789, pro-PRO50298, pro-PRO51592, pro-PRO1757, pro-PRO4421, pro-PRO9903, pro-PRO1106, pro-PRO1411, pro-PRO1486, pro-PRO1565, pro-PRO4399 or pro-PRO4404 polypeptide, mature PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, or pre-PRO179, pre-PRO181, pre-PRO244, pre-PRO247, pre-PRO269, pre-PRO293, pre-PRO298, pre-PRO339, pre-PRO341, pre-PRO347, pre-PRO531, pre-PRO537, pre-PRO718, pre-PRO773, pre-PRO860, pre-PRO871, pre-PRO872, pre-PRO813, pre-PRO828, pre-PRO1100, pre-PRO1114, pre-PRO1115, pre-PRO1126, pre-PRO1133, pre-PRO1154, pre-PRO1185, pre-PRO1194, pre-PRO1287, pre-PRO1291, pre-PRO1293, pre-PRO1310, pre-PRO1312, pre-PRO1335, pre-PRO1339, pre-PRO2155, pre-PRO1356, pre-PRO1385, pre-PRO1412, pre-PRO1487, pre-PRO1758, pre-PRO1779, pre-PRO1785, pre-PRO1889, pre-PRO90318, pre-PRO3434, pre-PRO3579, pre-PRO4322, pre-PRO4343, pre-PRO4347, pre-PRO4403, pre-PRO4976, pre-PRO260, pre-PRO6014, pre-PRO6027, pre-PRO6181, pre-PRO6714, pre-PRO9922, pre-PRO7179, pre-PRO7476, pre-PRO9824, pre-PRO19814, pre-PRO19836, pre-PRO20088, pre-PRO70789, pre-PRO50298, pre-PRO51592, pre-PRO1757, pre-PRO4421, pre-PRO9903, pre-PRO1106, pre-PRO1411, pre-PRO1486, pre-PRO1565, pre-PRO4399 or pre-PRO4404 polypeptide is purified by immunoaffinity chromatography using antibodies specific for the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide of interest. In general, an immunoaffinity column is constructed by covalently coupling the anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 polypeptide antibody to an activated chromatographic resin.

Polyclonal immunoglobulins are prepared from immune sera either by precipitation with ammonium sulfate or by purification on immobilized Protein A (Pharmacia LKB Biotechnology, Piscataway, N.J.). Likewise, monoclonal antibodies are prepared from mouse ascites fluid by ammonium sulfate precipitation or chromatography on immobilized Protein A. Partially purified immunoglobulin is covalently attached to a chromatographic resin such as CnBr-activated SEPHAROSE™ (Pharmacia LKB Biotechnology). The antibody is coupled to the resin, the resin is blocked, and the derivative resin is washed according to the manufacturer's instructions.

Such an immunoaffinity column is utilized in the purification of PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide by preparing a fraction from cells containing PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide in a soluble form. This preparation is derived by solubilization of the whole cell or of a subcellular fraction obtained via differential centrifugation by the addition of detergent or by other methods well known in the art. Alternatively, soluble polypeptide containing a signal sequence may be secreted in useful quantity into the medium in which the cells are grown.

A soluble PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide-containing preparation is passed over the immunoaffinity column, and the column is washed under conditions that allow the preferential absorbance of PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide (e.g., high ionic strength buffers in the presence of detergent). Then, the column is eluted under conditions that disrupt antibody/PRO179, antibody/PRO181, antibody/PRO244, antibody/PRO247, antibody/PRO269, antibody/PRO293, antibody/PRO298, antibody/PRO339, antibody/PRO341, antibody/PRO347, antibody/PRO531, antibody/PRO537, antibody/PRO718, antibody/PRO773, antibody/PRO860, antibody/PRO871, antibody/PRO872, antibody/PRO813, antibody/PRO828, antibody/PRO1100, antibody/PRO1114, antibody/PRO1115, antibody/PRO1126, antibody/PRO1133, antibody/PRO1154, antibody/PRO1185, antibody/PRO1194, antibody/PRO1287, antibody/PRO1291, antibody/PRO1293, antibody/PRO1310, antibody/PRO1312, antibody/PRO1335, antibody/PRO1339, antibody/PRO2155, antibody/PRO1356, antibody/PRO1385, antibody/PRO1412, antibody/PRO1487, antibody/PRO1758, antibody/PRO1779, antibody/PRO1785, antibody/PRO1889, antibody/PRO90318, antibody/PRO3434, antibody/PRO3579, antibody/PRO4322, antibody/PRO4343, antibody/PRO4347, antibody/PRO4403, antibody/PRO4976, antibody/PRO260, antibody/PRO6014, antibody/PRO6027, antibody/PRO6181, antibody/PRO6714, antibody/PRO9922, antibody/PRO7179, antibody/PRO7476, antibody/PRO9824, antibody/PRO19814, antibody/PRO19836, antibody/PRO20088, antibody/PRO70789, antibody/PRO50298, antibody/PRO51592, antibody/PRO1757, antibody/PRO4421, antibody/PRO9903, antibody/PRO1106, antibody/PRO1411, antibody/PRO1486, antibody/PRO1565, antibody/PRO4399 or antibody/PRO4404 polypeptide binding (e.g., a low pH buffer such as approximately pH 2-3, or a high concentration of a chaotrope such as urea or thiocyanate ion), and PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide is collected.

### Example 82

### Drug Screening

This invention is particularly useful for screening compounds by using PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptides or binding fragment thereof in any of a variety of drug screening techniques. The PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide or fragment employed in such a test may either be free in solution, affixed to a solid support, borne on a cell surface, or located intracellularly. One method of drug screening utilizes eukaryotic or prokaryotic host cells which are stably transformed with recombinant nucleic acids expressing the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide or fragment. Drugs are screened against such transformed cells in competitive binding assays. Such cells, either inviable or fixed form, can be used for standard binding assays. One may measure, for example, the formation of complexes between PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide or a fragment and the agent being tested. Alternatively, one can examine the diminution in complex formation between the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide and its target cell or target receptors caused by the agent being tested.

Thus, the present invention provides methods of screening for drugs or any other agents which can affect a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide-associated disease or disorder. These methods comprise contacting such an agent with an PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide or fragment thereof and assaying (I) for the presence of a complex between the agent and the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide or fragment, or (ii) for the presence of a complex between the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide or fragment and the cell, by methods well known in the art. In such competitive binding assays, the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide or fragment is typically labeled. After suitable incubation, free PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide or fragment is separated from that present in bound form, and the amount of free or uncomplexed label is a measure of the ability of the particular agent to bind to PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide or to interfere with the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide/cell complex.

Another technique for drug screening provides high throughput screening for compounds having suitable binding affinity to a polypeptide and is described in detail in WO 84/03564, published on Sep. 13, 1984. Briefly stated, large numbers of different small peptide test compounds are synthesized on a solid substrate, such as plastic pins or some other surface. As applied to a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the peptide test compounds are reacted with PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide and washed. Bound PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide is detected by methods well known in the art. Purified PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide can also be coated directly onto plates for use in the aforementioned drug screening techniques. In addition, non-neutralizing antibodies can be used to capture the peptide and immobilize it on the solid support.

This invention also contemplates the use of competitive drug screening assays in which neutralizing antibodies capable of binding PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide specifically compete with a test compound for binding to PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide or fragments thereof. In this manner, the antibodies can be used to detect the presence of any peptide which shares one or more antigenic determinants with PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide.

### Example 83

### Rational Drug Design

The goal of rational drug design is to produce structural analogs of biologically active polypeptide of interest (i.e., a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide) or of small molecules with which they interact, e.g., agonists, antagonists, or inhibitors. Any of these examples can be used to fashion drugs which are more active or stable forms of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide or which enhance or interfere with the function of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide in vivo (cf., Hodgson, Bio/Technology, 9: 19-21 (1991)).

In one approach, the three-dimensional structure of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, or of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide-inhibitor complex, is determined by x-ray crystallography, by computer modeling or, most typically, by a combination of the two approaches. Both the shape and charges of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide must be ascertained to elucidate the structure and to determine active site(s) of the molecule. Less often, useful information regarding the structure of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide may be gained by modeling based on the structure of homologous proteins. In both cases, relevant structural information is used to design analogous PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide-like molecules or to identify efficient inhibitors. Useful examples of rational drug design may include molecules which have improved activity or stability as shown by Braxton and Wells, Biochemistry, 31:7796-7801 (1992) or which act as inhibitors, agonists, or antagonists of native peptides as shown by Athauda et al., J. Biochem., 113:742-746 (1993).

It is also possible to isolate a target-specific antibody, selected by functional assay, as described above, and then to solve its crystal structure. This approach, in principle, yields a pharmacore upon which subsequent drug design can be based. It is possible to bypass protein crystallography altogether by generating anti-idiotypic antibodies (anti-ids) to a functional, pharmacologically active antibody. As a mirror image of a mirror image, the binding site of the anti-ids would be expected to be an analog of the original receptor. The anti-id could then be used to identify and isolate peptides from banks of chemically or biologically produced peptides. The isolated peptides would then act as the pharmacore.

By virtue of the present invention, sufficient amounts of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide may be made available to perform such analytical studies as X-ray crystallography. In addition, knowledge of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide amino acid sequence provided herein will provide guidance to those employing computer modeling techniques in place of or in addition to x-ray crystallography.

1-149. (canceled)

150. A method of identifying a phenotype associated with a disruption of a gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising:
(a) providing a non-human transgenic animal whose genome comprises a disruption of a gene which is an ortholog of a human gene that encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide;
(b) measuring a physiological characteristic of the non-human transgenic animal; and
(c) comparing the measured physiological characteristic with that of a gender matched wild-type animal, wherein the physiological characteristic of the non-human transgenic animal that differs from the physiological characteristic of the wild-type animal is identified as a phenotype resulting from the gene disruption in the non-human transgenic animal.

151. The method of claim 150, wherein the non-human transgenic animal is heterozygous for the disruption of a gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide.

152. The method of claim 150, wherein the phenotype exhibited by the non-human transgenic animal as compared with gender matched wild-type littermates is at least one of the following: a neurological disorder; a cardiovascular, endothelial or angiogenic disorder; an eye abnormality; an immunological disorder; an oncological disorder; a bone metabolic abnormality or disorder; a lipid metabolic disorder; or a developmental abnormality.

153. The method of claim 152, wherein the neurological disorder is an increased anxiety-like response during open field activity testing.

154. The method of claim 152, wherein the neurological disorder is a decreased anxiety-like response during open field activity testing.

155. The method of claim 152, wherein the neurological disorder is an abnormal circadian rhythm during home-cage activity testing.

156. The method of claim 152, wherein the neurological disorder is an enhanced motor coordination during inverted screen testing.

157. The method of claim 152, wherein the neurological disorder is an impaired motor coordination during inverted screen testing.

158. The method of claim 152, wherein the neurological disorder is depression, generalized anxiety disorders, attention deficit disorder, sleep disorder, hyperactivity disorder, obsessive compulsive disorder, schizophrenia, cognitive disorders, hyperalgesia or sensory disorders.

159. The method of claim 152, wherein the eye abnormality is a retinal abnormality.

160. The method of claim 152, wherein the eye abnormality is consistent with vision problems or blindness.

161. The method of claim 159, wherein the retinal abnormality is consistent with retinitis pigmentosa.

162. The method of claim 159, wherein the retinal abnormality is characterized by retinal degeneration or retinal dysplasia.

163. The method of claim 159, wherein the retinal abnormality is consistent with retinal dysplasia, various retinopathies, including retinopathy of prematurity, retrolental fibroplasia, neovascular glaucoma, age-related macular degeneration, diabetic macular edema, corneal neovascularization, corneal graft neovascularization, corneal graft rejection, retinalkhoroidal neovascularization, neovascularization of the angle (rubeosis), ocular neovascular disease, vascular restenosis, arteriovenous malformations (AVM), meningioma, hemangioma, angiofibroma, thyroid hyperplasias (including Grave's disease), corneal and other tissue transplantation, retinal artery obstruction or occlusion; retinal degeneration causing secondary atrophy of the retinal vasculature, retinitis pigmentosa, macular dystrophies, Stargardt's disease, congenital stationary night blindness, choroideremia, gyrate atrophy, Leber's congenital amaurosis, retinoschisis disorders, Wagner's syndrome, Usher syndromes, Zellweger syndrome, Saldino-Mainzer syndrome, Senior-Loken syndrome, Bardet-Biedl syndrome, Alport's syndrome, Alstrom's syndrome, Cockayne's syndrome, dysplaisa spondyloepiphysaria congentia, Flynn-Aird syndrome, Friedreich ataxia, Hallgren syndrome, Marshall syndrome, Albers-Schnoberg disease, Refsum's disease, Kearns-Sayre syndrome, Waardenburg's syndrome, Alagile syndrome, myotonic dystrophy, olivopontocerebellar atrophy, Pierre-Marie dunsdrome, Stickler syndrome, carotinemeia, cystinosis, Wolfram syndrome, Bassen-Kornzweig syndrome, abetalipoproteinemia, incontinentia pigmenti, Batten's disease, mucopolysaccharidoses, homocystinuria, or mannosidosis.

164. The method of claim 152, wherein the eye abnormality is a cataract.

165. The method of claim 164, wherein the cataract is consistent with systemic diseases such as human Down's syndrome, Hallerman-Streiff syndrome, Lowe syndrome, galactosemia, Marfan syndrome, Trismoy 13-15, Alport syndrome, myotonic dystrophy, Fabry disease, hypoparathroidism or Conradi syndrome.

166. The method of claim 152, wherein the developmental abnormality comprises embryonic lethality or reduced viability.

167. The method of claim 152, wherein the cardiovascular, endothelial or angiogenic disorders are arterial diseases, such as diabetes mellitus; papilledema; optic atrophy; atherosclerosis; angina; myocardial infarctions such as acute myocardial infarctions, cardiac hypertrophy, and heart failure such as congestive heart failure; hypertension; inflammatory vasculitides; Reynaud's disease and Reynaud's phenomenon; aneurysms and arterial restenosis; venous and lymphatic disorders such as thrombophlebitis, lymphangitis, and lymphedema; peripheral vascular disease; cancer such as vascular tumors, e.g., hemangioma (capillary and cavernous), glomus tumors, telangiectasia, bacillary angiomatosis, hemangioendothelioma, angiosarcoma, haemangiopericytoma, Kaposi's sarcoma, lymphangioma, and lymphangiosarcoma; tumor angiogenesis; trauma such as wounds, burns, and other injured tissue, implant fixation, scarring; ischemia reperfusion injury; rheumatoid arthritis; cerebrovascular disease; renal diseases such as acute renal failure, or osteoporosis.

168. The method of claim 152, wherein the immunological disorders are systemic lupus erythematosis; rheumatoid arthritis; juvenile chronic arthritis; spondyloarthropathies; systemic sclerosis (scleroderma); idiopathic inflammatory myopathies (dermatomyositis, polymyositis); Sjögren's syndrome; systemic vasculitis; sarcoidosis; autoimmune hemolytic anemia (immune pancytopenia, paroxysmal nocturnal hemoglobinuria); autoimmune thrombocytopenia (idiopathic thrombocytopenic purpura, immune-mediated thrombocytopenia); thyroiditis (Grave's disease, Hashimoto's thyroiditis, juvenile lymphocytic thyroiditis, atrophic thyroiditis); diabetes mellitus; immune-mediated renal disease (glomerulonephritis, tubulointerstitial nephritis); demyelinating diseases of the central and peripheral nervous systems such as multiple sclerosis, idiopathic demyelinating polyneuropathy or Guillain-Barré syndrome, and chronic inflammatory demyelinating polyneuropathy; hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis; inflammatory bowel disease (ulcerative colitis: Crohn's disease); gluten-sensitive enteropathy, and Whipple's disease; autoimmune or immune-mediated skin diseases including bullous skin diseases, erythema multiforme and contact dermatitis, psoriasis; allergic diseases such as asthma, allergic rhinitis, atopic dermatitis, food hypersensitivity and urticaria; immunologic diseases of the lung such as eosinophilic pneumonias, idiopathic pulmonary fibrosis and hypersensitivity pneumonitis; or transplantation associated diseases including graft rejection and graft-versus-host disease.

169. The method of claim 152, wherein the bone metabolic abnormality or disorder is arthritis, osteoporosis or osteopetrosis.

170. The method of claim 150, wherein the non-human transgenic animal exhibits at least one of the following physiological characteristics compared with gender matched wild-type littermates: increased anxiety-like response during open field testing; decreased anxiety-like response during open field activity testing; hyperactivity with increased rearing and hole poke activity during open field testing; hypoactivity with decreased rearing and hole poke activity during open field testing; increased exploratory activity during open-field testing; decreased exploratory activity during open-field testing; augmentation of circadian rhythm; abnormal circadian rhythm during home-cage activity testing including decreased ambulatory counts; abnormal circadian rhythm during home-cage activity testing including increased ambulatory counts; enhanced circadian rhythm; increased stress induced hyperthermia with increased stress response; increased resistance to stress induced hyperthermia; decreased resistance to stress induced hyperthermia; impaired motor coordination during inverted screen testing; increased depressive-like response during tail suspension testing; decreased depressive-like response during tail suspension testing; decreased startle response during prepulse inhibition testing; no startle response indicating deafness; reduced latency to respond in hot plate testing; increased pain perception in hot plate testing; prolonged latency to respond in hot plate testing; decreased pain perception in hot plate testing; straub tails during functional observational battery testing; opthamological abnormalities; attenuated retinal arteries; optic nerve abnormalities; retinal degeneration; retinal depigmentation; cataracts; decreased heart rate; decreased mean systolic blood pressure; increased mean systolic blood pressure; increased insulin sensitivity; increased mean fasting serum glucose levels; decreased mean serum glucose levels; increased mean serum cholesterol levels; decreased mean serum cholesterol levels; increased mean serum triglyceride levels; decreased mean serum triglyceride levels; enhanced glucose tolerance; impaired glucose tolerance; decreased mean serum insulin levels; increased uric acid levels; ketonemia; increased mean serum phosphorous levels; increased mean serum potassium levels; increased mean serum alkaline phosphatase levels; decreased mean serum alkaline phosphatase levels; blood in the urine; increased nitrituria; ketonuria; decreased mean serum albumin; decreased mean percentage of natural killer cells; abnormal leukocyte count; increased mean percentage of CD4 cells; decreased mean percentage of CD4 cells; increased mean percentage of B cells in peripheral blood; increase in CD4+ and CD8+ cells with decrease in B cells; decreased B cells and less CD11 blow cells in peritoneum; increased mean percentage B cells in spleen, lymph nodes and Peyer's patches; increase in activated/memory T cells by CD25+ staining and CD62L/CD44 staining; increase in activated/memory T cells in spleen; decreased mean percentage of CD8+ cells; increase total white blood cells (increase in neutrophils, lymphocytes, monocytes and basophils); decreased lymphocytes; increased mean absolute monocyte count; increased mean absolute neutrophil count; decreased mean absolute monocyte count; decreased mean serum IgM, IgA, IgG3, IgG2b and IgG2a levels; decreased mean serum IgG3 levels; decreased mean serum IgM levels; decreased mean serum IgG2a levels; decreased mean serum IgG3 and IgM levels; increase in mean serum IgM levels; increase in mean serum IgG2a levels; increase in mean serum IgG2b levels; anemia; decreased red blood cell count, decreased hemoglobin and decreased hematocrit; increased mean corpuscular volume; increased mean corpuscular hemoglobin; decreased mean corpuscular volume; decreased mean corpuscular hemoglobin; increased red blood cell distribution width and mean platelet volume; decreased red blood cell distribution width; skewed ratios of B220med/CD23− and B220+/CD11− low/CD23− cells after peritoneal lavage; increased CD25 T cells in lymph node and spleen; increased CD38 non-lymphoid cells in Peyer's patches; increased CD23 B cells (peritoneal); decreased percentage of CD4/CD8 DP cells and increased percentage of TCRB+ cells in thymus; decrease in Peyer's patch B cells; reduced number of TCRB+ CD38+ activated T cells in Peyer's patches; increased splenic CD25+ cells and peritoneal CD23 B cells; increased mean platelet count; decreased mean platelet count; decreased mean serum IgG1 response to an ovalbumin challenge; decreased mean serum IgG2a response to an ovalbumin challenge; increased mean serum IgG2a response to an ovalbumin challenge; increased mean serum MCP-1 response to a LPS challenge; increased mean serum TNF-alpha response to a LPS challenge; increased mean serum IL-6 response to a LPS challenge; increased skin fibroblast proliferation; decreased skin fibroblast proliferation; increased mean percent of total body fat and total fat mass; increased mean body weight; increased mean body length; increased total tissue mass (TTM); increased lean body mass (LBM); increased femoral bone mineral density (BMD); increased vertebral bone mineral density (BMD); increased BMC/LBM ratio; increased bone mineral density (BMD); increased total body volumetric bone mineral density (vBMD); increased bone mineral content (BMC); increased mean femoral midshaft cortical thickness and cross-sectional area; increased mean vertebral trabecular bone volume, number and connectivity density; decreased mean percent of total body fat and total fat mass; decreased mean body weight; decreased mean body length; decreased total tissue mass (TTM); decreased lean body mass (LBM); decreased femoral bone mineral density (BMD); decreased vertebral bone mineral density (BMD); decreased BMC/LBM ratio; decreased bone mineral density (BMD); decreased bone mineral content (BMC); decreased volumetric bone mineral density (vBMD); decreased mean femoral midshaft cortical thickness and cross-sectional area; decreased mean vertebral trabecular bone volume, number and connectivity density; myeloid hyperplasia in bone marrow; osteopetrosis with increased bone mineralization; increase in abdominal fat depots; chronic-active arthritis; proliferative chondrapathy and arthropathy; proliferation of cartilage in femoral tibia joints; chondrous metaplasia of cruciate ligaments and perichondral connective tissues; chronic active dermatitis; chronic active inflammation in periarticular tissues; chronic inflammation in various tissues; myeloid hyperplasia in femur and sternum with associated erythroid hyperplasia in the spleen; increased spleen weight; impaired gastrointestinal motility; thymic atrophy; thymic T cell lymphoma; growth retardation; development abnormalities; stunted growth with general reduction in all organ size; growth retardation with reduced viability; and embryonic lethality.

171. An isolated cell derived from a non-human transgenic animal whose genome comprises disruption of a gene which is an ortholog of a human gene that encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide.

172. The isolated cell of claim 171 which is a murine cell.

173. The isolated cell of claim 172, wherein the murine cell is an embryonic stem cell.

174. The isolated cell of claim 171, wherein the non-human transgenic animal exhibits at least one of the following phenotypes compared with gender matched wild-type littermates: a neurological disorder; a cardiovascular, endothelial or angiogenic disorder; an eye abnormality; an immunological disorder; an oncological disorder; a bone metabolic abnormality or disorder; a lipid metabolic disorder; or a developmental abnormality.

175. A method of identifying an agent that modulates a phenotype associated with a disruption of a gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising:
(a) providing a non-human transgenic animal whose genome comprises a disruption of a gene which is an ortholog of a human gene that encodes for the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide;
(b) measuring a physiological characteristic of the non-human transgenic animal of (a);
(c) comparing the measured physiological characteristic of (b) with that of a gender matched wild-type animal, wherein the physiological characteristic of the non-human transgenic animal that differs from the physiological characteristic of the wild-type animal is identified as a phenotype resulting from the gene disruption in the non-human transgenic animal;
(d) administering a test agent to the non-human transgenic animal of (a); and
(e) determining whether the test agent modulates the identified phenotype associated with gene disruption in the non-human transgenic animal.

176. The method of claim 175, wherein the phenotype associated with the gene disruption comprises a neurological disorder; a cardiovascular, endothelial or angiogenic disorder; an eye abnormality; an immunological disorder; an oncological disorder; a bone metabolic abnormality or disorder; a lipid metabolic disorder; or a developmental abnormality.

177. The method of claim 176, wherein the neurological disorder is an increased anxiety-like response during open field activity testing.

178. The method of claim 176, wherein the neurological disorder is a decreased anxiety-like response during open field activity testing.

179. The method of claim 176, wherein the neurological disorder is an abnormal circadian rhythm during home-cage activity testing.

180. The method of claim 176, wherein the neurological disorder is an enhanced motor coordination during inverted screen testing.

181. The method of claim 176, wherein the neurological disorder is an impaired motor coordination during inverted screen testing.

182. The method of claim 176, wherein the neurological disorder is depression, generalized anxiety disorders, attention deficit disorder, sleep disorder, hyperactivity disorder, obsessive compulsive disorder, schizophrenia, cognitive disorders, hyperalgesia or sensory disorders.

183. The method of claim 176, wherein the eye abnormality is a retinal abnormality.

184. The method of claim 176, wherein the eye abnormality is consistent with vision problems or blindness.

185. The method of claim 183, wherein the retinal abnormality is consistent with retinitis pigmentosa.

186. The method of claim 183, wherein the retinal abnormality is characterized by retinal degeneration or retinal dysplasia.

187. The method of claim 183, wherein the retinal abnormality is consistent with retinal dysplasia, various retinopathies, including retinopathy of prematurity, retrolental fibroplasia, neovascular glaucoma, age-related macular degeneration, diabetic macular edema, corneal neovascularization, corneal graft neovascularization, corneal graft rejection, retinal/choroidal neovascularization, neovascularization of the angle (rubeosis), ocular neovascular disease, vascular restenosis, arteriovenous malformations (AVM), meningioma, hemangioma, angiofibroma, thyroid hyperplasias (including Grave's disease), corneal and other tissue transplantation, retinal artery obstruction or occlusion; retinal degeneration causing secondary atrophy of the retinal vasculature, retinitis pigmentosa, macular dystrophies, Stargardt's disease, congenital stationary night blindness, choroideremia, gyrate atrophy, Leber's congenital amaurosis, retinoschisis disorders, Wagner's syndrome, Usher syndromes, Zellweger syndrome, Saldino-Mainzer syndrome, Senior-Loken syndrome, Bardet-Biedl syndrome, Alport's syndrome, Alstrom's syndrome, Cockayne's syndrome, dysplaisa spondyloepiphysaria congentia, Flynn-Aird syndrome, Friedreich ataxia, Hallgren syndrome, Marshall syndrome, Albers-Schnoberg disease, Refsum's disease, Kearns-Sayre syndrome, Waardenburg's syndrome, Alagile syndrome, myotonic dystrophy, olivopontocerebellar atrophy, Pierre-Marie dunsdrome, Stickler syndrome, carotinemeia, cystinosis, Wolfram syndrome, Bassen-Kornzweig syndrome, abetalipoproteinemia, incontinentia pigmenti, Batten's disease, mucopolysaccharidoses, homocystinuria, or mannosidosis.

188. The method of claim 176, wherein the eye abnormality is a cataract.

189. The method of claim 188, wherein the cataract is consistent with systemic diseases such as human Down's syndrome, Hallerman-Streiff syndrome, Lowe syndrome, galactosemia, Marfan syndrome, Trismoy 13-15, Alport syndrome, myotonic dystrophy, Fabry disease, hypoparathroidism or Conradi syndrome.

190. The method of claim 176, wherein the developmental abnormality comprises embryonic lethality or reduced viability.

191. The method of claim 176, wherein the cardiovascular, endothelial or angiogenic disorders are arterial diseases, such as diabetes mellitus; papilledema; optic atrophy; atherosclerosis; angina; myocardial infarctions such as acute myocardial infarctions, cardiac hypertrophy, and heart failure such as congestive heart failure; hypertension; inflammatory vasculitides; Reynaud's disease and Reynaud's phenomenon; aneurysms and arterial restenosis; venous and lymphatic disorders such as thrombophlebitis, lymphangitis, and lymphedema; peripheral vascular disease; cancer such as vascular tumors, e.g., hemangioma (capillary and cavernous), glomus tumors, telangiectasia, bacillary angiomatosis, hemangioendothelioma, angiosarcoma, haemangiopericytoma, Kaposi's sarcoma, lymphangioma, and lymphangiosarcoma; tumor angiogenesis; trauma such as wounds, burns, and other injured tissue, implant fixation, scarring; ischemia reperfusion injury; rheumatoid arthritis; cerebrovascular disease; renal diseases such as acute renal failure, or osteoporosis.

192. The method of claim 176, wherein the immunological disorders are systemic lupus erythematosis; rheumatoid arthritis; juvenile chronic arthritis spondyloarthropathies; systemic sclerosis (scleroderma); idiopathic inflammatory myopathies (dermatomyositis, polymyositis); Sjögren's syndrome; systemic vasculitis; sarcoidosis; autoimmune hemolytic anemia (immune pancytopenia, paroxysmal nocturnal hemoglobinuria); autoimmune thrombocytopenia (idiopathic thrombocytopenic purpura, immune-mediated thrombocytopenia); thyroiditis (Grave's disease, Hashimoto's thyroiditis, juvenile lymphocytic thyroiditis, atrophic thyroiditis); diabetes mellitus; immune-mediated renal disease (glomerulonephritis, tubulointerstitial nephritis); demyelinating diseases of the central and peripheral nervous systems such as multiple sclerosis, idiopathic demyelinating polyneuropathy or Guillain-Barré syndrome, and chronic inflammatory demyelinating polyneuropathy; hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis; inflammatory bowel disease (ulcerative colitis: Crohn's disease); gluten-sensitive enteropathy, and Whipple's disease; autoimmune or immune-mediated skin diseases including bullous skin diseases, erythema multiforme and contact dermatitis, psoriasis; allergic diseases such as asthma, allergic rhinitis, atopic dermatitis, food hypersensitivity and urticaria; immunologic diseases of the lung such as eosinophilic pneumonia, idiopathic pulmonary fibrosis and hypersensitivity pneumonitis; or transplantation-associated diseases including graft rejection and graft-versus-host disease.

193. The method of claim 176, wherein said bone metabolic abnormality or disorder is arthritis, osteoporosis or osteopetrosis.

194. The method of claim 175, wherein the non-human transgenic animal exhibits at least one of the following physiological characteristics compared with gender matched wild-type littermates: increased anxiety-like response during open field testing; decreased anxiety-like response during open field activity testing; hyperactivity with increased rearing and hole poke activity during open field testing; hypoactivity with decreased rearing and hole poke activity during open field testing; increased exploratory activity during open-field testing; decreased exploratory activity during open-field testing; augmentation of circadian rhythm; abnormal circadian rhythm during home-cage activity testing including decreased ambulatory counts; abnormal circadian rhythm during home-cage activity testing including increased ambulatory counts; enhanced circadian rhythm; increased stress induced hyperthermia with increased stress response; increased resistance to stress induced hyperthermia; decreased resistance to stress induced hyperthermia; impaired motor coordination during inverted screen testing; increased depressive-like response during tail suspension testing; decreased depressive-like response during tail suspension testing; decreased startle response during prepulse inhibition testing; no startle response indicating deafness; reduced latency to respond in hot plate testing; increased pain perception in hot plate testing; prolonged latency to respond in hot plate testing; decreased pain perception in hot plate testing; straub tails during functional observational battery testing; opthamological abnormalities; attenuated retinal arteries; optic nerve abnormalities; retinal degeneration; retinal depigmentation; cataracts; decreased heart rate; decreased mean systolic blood pressure; increased mean systolic blood pressure; increased insulin sensitivity; increased mean fasting serum glucose levels; decreased mean serum glucose levels; increased mean serum cholesterol levels; decreased mean serum cholesterol levels; increased mean serum triglyceride levels; decreased mean serum triglyceride levels; enhanced glucose tolerance; impaired glucose tolerance; decreased mean serum insulin levels; increased uric acid levels; ketonemia; increased mean serum phosphorous levels; increased mean serum potassium levels; increased mean serum alkaline phosphatase levels; decreased mean serum alkaline phosphatase levels; blood in the urine; increased nitrituria; ketonuria; decreased mean serum albumin; decreased mean percentage of natural killer cells; abnormal leukocyte count; increased mean percentage of CD4 cells; decreased mean percentage of CD4 cells; increased mean percentage of B cells in peripheral blood; increase in CD4+ and CD8+ cells with decrease in B cells; decreased B cells and less CD11 blow cells in peritoneum; increased mean percentage B cells in spleen, lymph nodes and Peyer's patches; increase in activated/memory T cells by CD25+ staining and CD62L/CD44 staining; increase in activated/memory T cells in spleen; decreased mean percentage of CD8+ cells; increase total white blood cells (increase in neutrophils, lymphocytes, monocytes and basophils); decreased lymphocytes; increased mean absolute monocyte count; increased mean absolute neutrophil count; decreased mean absolute monocyte count; decreased mean serum IgM, IgA, IgG3, IgG2b and IgG2a levels; decreased mean serum IgG3 levels; decreased mean serum IgM levels; decreased mean serum IgG2a levels; decreased mean serum IgG3 and IgM levels; increase in mean serum IgM levels; increase in mean serum IgG2a levels; increase in mean serum IgG2b levels; anemia; decreased red blood cell count, decreased hemoglobin and decreased hematocrit; increased mean corpuscular volume; increased mean corpuscular hemoglobin; decreased mean corpuscular volume; decreased mean corpuscular hemoglobin; increased red blood cell distribution width and mean platelet volume; decreased red blood cell distribution width; skewed ratios of B220med/CD23− and B220+/CD11− low/CD23− cells after peritoneal lavage; increased CD25 T cells in lymph node and spleen; increased CD38 non-lymphoid cells in Peyer's patches; increased CD23 B cells (peritoneal); decreased percentage of CD4/CD8 DP cells and increased percentage of TCRB+ cells in thymus; decrease in Peyer's patch B cells; reduced number of TCRB+ CD38+ activated T cells in Peyer's patches; increased splenic CD25+ cells and peritoneal CD23 B cells; increased mean platelet count; decreased mean platelet count; decreased mean serum IgG1 response to an ovalbumin challenge; decreased mean serum IgG2a response to an ovalbumin challenge; increased mean serum IgG2a response to an ovalbumin challenge; increased mean serum MCP-1 response to a LPS challenge; increased mean serum TNF-alpha response to a LPS challenge; increased mean serum IL-6 response to a LPS challenge; increased skin fibroblast proliferation; decreased skin fibroblast proliferation; increased mean percent of total body fat and total fat mass; increased mean body weight; increased mean body length; increased total tissue mass (TTM); increased lean body mass (LBM); increased femoral bone mineral density (BMD); increased vertebral bone mineral density (BMD); increased BMC/LBM ratio; increased bone mineral density (BMD); increased total body volumetric bone mineral density (vBMD); increased bone mineral content (BMC); increased mean femoral midshaft cortical thickness and cross-sectional area; increased mean vertebral trabecular bone volume, number and connectivity density; decreased mean percent of total body fat and total fat mass; decreased mean body weight; decreased mean body length; decreased total tissue mass (TTM); decreased lean body mass (LBM); decreased femoral bone mineral density (BMD); decreased vertebral bone mineral density (BMD); decreased BMC/LBM ratio; decreased bone mineral density (BMD); decreased bone mineral content (BMC); decreased volumetric bone mineral density (vBMD); decreased mean femoral midshaft cortical thickness and cross-sectional area; decreased mean vertebral trabecular bone volume, number and connectivity density; myeloid hyperplasia in bone marrow; osteopetrosis with increased bone mineralization; increase in abdominal fat depots; chronic-active arthritis; proliferative chondrapathy and arthropathy; proliferation of cartilage in femoral tibia joints; chondrous metaplasia of cruciate ligaments and perichondral connective tissues; chronic active dermatitis; chronic active inflammation in periarticular tissues; chronic inflammation in various tissues; myeloid hyperplasia in femur and sternum with associated erythroid hyperplasia in the spleen; increased spleen weight; impaired gastrointestinal motility; thymic atrophy; thymic T cell lymphoma; growth retardation; development abnormalities; stunted growth with general reduction in all organ size; growth retardation with reduced viability; and embryonic lethality.

195. An agent identified by the method of claim 175.

196. The agent of claim 195 which is an agonist or antagonist of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide.

197. The agent of claim 196, wherein the agonist is an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody.

198. The agent of claim 196, wherein the antagonist is an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody.

199. A method of identifying an agent that modulates a physiological characteristic associated with a disruption of a gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising:
(a) providing a non-human transgenic animal whose genome comprises a disruption of a gene which is an ortholog of a human gene that encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide;
(b) measuring a physiological characteristic exhibited by the non-human transgenic animal of (a);
(c) comparing the measured physiological characteristic of (b) with that of a gender matched wild-type animal, wherein the physiological characteristic exhibited by the non-human transgenic animal that differs from the physiological characteristic exhibited by the wild-type animal is identified as a physiological characteristic associated with gene disruption;
(d) administering a test agent to the non-human transgenic animal of (a); and
(e) determining whether the physiological characteristic associated with gene disruption is modulated.

200. The method of claim 199, wherein the non-human transgenic animal exhibits at least one of the following physiological characteristics compared with gender matched wild-type littermates: increased anxiety-like response during open field testing; decreased anxiety-like response during open field activity testing; hyperactivity with increased rearing and hole poke activity during open field testing; hypoactivity with decreased rearing and hole poke activity during open field testing; increased exploratory activity during open-field testing; decreased exploratory activity during open-field testing; augmentation of circadian rhythm; abnormal circadian rhythm during home-cage activity testing including decreased ambulatory counts; abnormal circadian rhythm during home-cage activity testing including increased ambulatory counts; enhanced circadian rhythm; increased stress induced hyperthermia with increased stress response; increased resistance to stress induced hyperthermia; decreased resistance to stress induced hyperthermia; impaired motor coordination during inverted screen testing; increased depressive-like response during tail suspension testing; decreased depressive-like response during tail suspension testing; decreased startle response during prepulse inhibition testing; no startle response indicating deafness; reduced latency to respond in hot plate testing; increased pain perception in hot plate testing; prolonged latency to respond in hot plate testing; decreased pain perception in hot plate testing; straub tails during functional observational battery testing; opthamological abnormalities; attenuated retinal arteries; optic nerve abnormalities; retinal degeneration; retinal depigmentation; cataracts; decreased heart rate; decreased mean systolic blood pressure; increased mean systolic blood pressure; increased insulin sensitivity; increased mean fasting serum glucose levels; decreased mean serum glucose levels; increased mean serum cholesterol levels; decreased mean serum cholesterol levels; increased mean serum triglyceride levels; decreased mean serum triglyceride levels; enhanced glucose tolerance; impaired glucose tolerance; decreased mean serum insulin levels; increased uric acid levels; ketonemia; increased mean serum phosphorous levels; increased mean serum potassium levels; increased mean serum alkaline phosphatase levels; decreased mean serum alkaline phosphatase levels; blood in the urine; increased nitrituria; ketonuria; decreased mean serum albumin; decreased mean percentage of natural killer cells; abnormal leukocyte count; increased mean percentage of CD4 cells; decreased mean percentage of CD4 cells; increased mean percentage of B cells in peripheral blood; increase in CD4+ and CD8+ cells with decrease in B cells; decreased B cells and less CD11 blow cells in peritoneum; increased mean percentage B cells in spleen, lymph nodes and Peyer's patches; increase in activated/memory T cells by CD25+ staining and CD62L/CD44 staining; increase in activated/memory T cells in spleen; decreased mean percentage of CD8+ cells; increase total white blood cells (increase in neutrophils, lymphocytes, monocytes and basophils); decreased lymphocytes; increased mean absolute monocyte count; increased mean absolute neutrophil count; decreased mean absolute monocyte count; decreased mean serum IgM, IgA, IgG3, IgG2b and IgG2a levels; decreased mean serum IgG3 levels; decreased mean serum IgM levels; decreased mean serum IgG2a levels; decreased mean serum IgG3 and IgM levels; increase in mean serum IgM levels; increase in mean serum IgG2a levels; increase in mean serum IgG2b levels; anemia; decreased red blood cell count, decreased hemoglobin and decreased hematocrit; increased mean corpuscular volume; increased mean corpuscular hemoglobin; decreased mean corpuscular volume; decreased mean corpuscular hemoglobin; increased red blood cell distribution width and mean platelet volume; decreased red blood cell distribution width; skewed ratios of B220med/CD23− and B220+/CD11− low/CD23− cells after peritoneal lavage; increased CD25 T cells in lymph node and spleen; increased CD38 non-lymphoid cells in Peyer's patches; increased CD23 B cells (peritoneal); decreased percentage of CD4/CD8 DP cells and increased percentage of TCRB+ cells in thymus; decrease in Peyer's patch B cells; reduced number of TCRB+ CD38+ activated T cells in Peyer's patches; increased splenic CD25+ cells and peritoneal CD23 B cells; increased mean platelet count; decreased mean platelet count; decreased mean serum IgG1 response to an ovalbumin challenge; decreased mean serum IgG2a response to an ovalbumin challenge; increased mean serum IgG2a response to an ovalbumin challenge; increased mean serum MCP-1 response to a LPS challenge; increased mean serum TNF-alpha response to a LPS challenge; increased mean serum IL-6 response to a LPS challenge; increased skin fibroblast proliferation; decreased skin fibroblast proliferation; increased mean percent of total body fat and total fat mass; increased mean body weight; increased mean body length; increased total tissue mass (TTM); increased lean body mass (LBM); increased femoral bone mineral density (BMD); increased vertebral bone mineral density (BMD); increased BMC/LBM ratio; increased bone mineral density (BMD); increased total body volumetric bone mineral density (vBMD); increased bone mineral content (BMC); increased mean femoral midshaft cortical thickness and cross-sectional area; increased mean vertebral trabecular bone volume, number and connectivity density; decreased mean percent of total body fat and total fat mass; decreased mean body weight; decreased mean body length; decreased total tissue mass (TTM); decreased lean body mass (LBM); decreased femoral bone mineral density (BMD); decreased vertebral bone mineral density (BMD); decreased BMC/LBM ratio; decreased bone mineral density (BMD); decreased bone mineral content (BMC); decreased volumetric bone mineral density (vBMD); decreased mean femoral midshaft cortical thickness and cross-sectional area; decreased mean vertebral trabecular bone volume, number and connectivity density; myeloid hyperplasia in bone marrow; osteopetrosis with increased bone mineralization; increase in abdominal fat depots; chronic-active arthritis; proliferative chondrapathy and arthropathy; proliferation of cartilage in femoral tibia joints; chondrous metaplasia of cruciate ligaments and perichondral connective tissues; chronic active dermatitis; chronic active inflammation in periarticular tissues; chronic inflammation in various tissues; myeloid hyperplasia in femur and sternum with associated erythroid hyperplasia in the spleen; increased spleen weight; impaired gastrointestinal motility; thymic atrophy; thymic T cell lymphoma; growth retardation; development abnormalities; stunted growth with general reduction in all organ size; growth retardation with reduced viability; and embryonic lethality.

201. An agent identified by the method of claim 199.

202. The agent of claim 201 which is an agonist or antagonist of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide.

203. The agent of claim 202, wherein the agonist is an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody.

204. The agent of claim 202, wherein the antagonist is an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody.

205. A method of identifying an agent which modulates a behavior associated with a disruption of a gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising:
(a) providing a non-human transgenic animal whose genome comprises a disruption of a gene which is an ortholog of a human gene that encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide;
(b) observing the behavior exhibited by the non-human transgenic animal of (a);
(c) comparing the observed behavior of (b) with that of a gender matched wild-type animal, wherein the observed behavior exhibited by the non-human transgenic animal that differs from the observed behavior exhibited by the wild-type animal is identified as a behavior associated with gene disruption;
(d) administering a test agent to the non-human transgenic animal of (a); and
(e) determining whether the agent modulates the behavior associated with gene disruption.

206. The method of claim 205, wherein the behavior is an increased anxiety-like response during open field activity testing.

207. The method of claim 205, wherein the behavior is a decreased anxiety-like response during open field activity testing.

208. The method of claim 205, wherein the behavior is an abnormal circadian rhythm during home-cage activity testing.

209. The method of claim 205, wherein the behavior is an enhanced motor coordination during inverted screen testing.

210. The method of claim 205, wherein the behavior is an impaired motor coordination during inverted screen testing.

211. The method of claim 205, wherein the behavior is depression, generalized anxiety disorders, attention deficit disorder, sleep disorder, hyperactivity disorder, obsessive compulsive disorder, schizophrenia, cognitive disorders, hyperalgesia or sensory disorders.

212. An agent identified by the method of claim 205.

213. The agent of claim 212 which is an agonist or antagonist of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide.

214. The agent of claim 213, wherein the agonist is an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody.

215. The agent of claim 213, wherein the antagonist is an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody.

216. A method of identifying an agent that ameliorates or modulates a neurological disorder; a cardiovascular, endothelial or angiogenic disorder; an eye abnormality; an immunological disorder; an oncological disorder; a bone metabolic abnormality or disorder; a lipid metabolic disorder; or a developmental abnormality associated with a disruption in a gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising:
(a) providing a non-human transgenic animal whose genome comprises a disruption of a gene which is an ortholog of a human gene that encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide;
(b) administering a test agent to said non-human transgenic animal; and
(c) determining whether said test agent ameliorates or modulates the neurological disorder; cardiovascular, endothelial or angiogenic disorder; eye abnormality; immunological disorder; oncological disorder; bone metabolic abnormality or disorder; lipid metabolic disorder; or developmental abnormality in the non-human transgenic animal.

217. The method of claim 216, wherein the neurological disorder is an increased anxiety-like response during open field activity testing.

218. The method of claim 216, wherein the neurological disorder is a decreased anxiety-like response during open field activity testing.

219. The method of claim 216, wherein the neurological disorder is an abnormal circadian rhythm during home-cage activity testing.

220. The method of claim 216, wherein the neurological disorder is an enhanced motor coordination during inverted screen testing.

221. The method of claim 216, wherein the neurological disorder is an impaired motor coordination during inverted screen testing.

222. The method of claim 216, wherein the neurological disorder is depression, generalized anxiety disorders, attention deficit disorder, sleep disorder, hyperactivity disorder, obsessive compulsive disorder, schizophrenia, cognitive disorders, hyperalgesia or sensory disorders.

223. The method of claim 216, wherein the eye abnormality is a retinal abnormality.

224. The method of claim 216, wherein the eye abnormality is consistent with vision problems or blindness.

225. The method of claim 223, wherein the retinal abnormality is consistent with retinitis pigmentosa.

226. The method of claim 223, wherein the retinal abnormality is characterized by retinal degeneration or retinal dysplasia.

227. The method of claim 223, wherein the retinal abnormality is consistent with retinal dysplasia, various retinopathies, including retinopathy of prematurity, retrolental fibroplasia, neovascular glaucoma, age-related macular degeneration, diabetic macular edema, corneal neovascularization, corneal graft neovascularization, corneal graft rejection, retinal/choroidal neovascularization, neovascularization of the angle (rubeosis), ocular neovascular disease, vascular restenosis, arteriovenous malformations (AVM), meningioma, hemangioma, angiofibroma, thyroid hyperplasias (including Grave's disease), corneal and other tissue transplantation, retinal artery obstruction or occlusion; retinal degeneration causing secondary atrophy of the retinal vasculature, retinitis pigmentosa, macular dystrophies, Stargardt's disease, congenital stationary night blindness, choroideremia, gyrate atrophy, Leber's congenital amaurosis, retinoschisis disorders, Wagner's syndrome, Usher syndromes, Zellweger syndrome, Saldino-Mainzer syndrome, Senior-Loken syndrome, Bardet-Biedl syndrome, Alport's syndrome, Alstrom's syndrome, Cockayne's syndrome, dysplaisa spondyloepiphysaria congentia, Flynn-Aird syndrome, Friedreich ataxia, Hallgren syndrome, Marshall syndrome, Albers-Schnoberg disease, Refsum's disease, Kearns-Sayre syndrome, Waardenburg's syndrome, Alagile syndrome, myotonic dystrophy, olivopontocerebellar atrophy, Pierre-Marie dunsdrome, Stickler syndrome, carotinemeia, cystinosis, Wolfram syndrome, Bassen-Kornzweig syndrome, abetalipoproteinemia, incontinentia pigmenti, Batten's disease, mucopolysaccharidoses, homocystinuria, or mannosidosis.

228. The method of claim 216, wherein the eye abnormality is a cataract.

229. The method of claim 228, wherein the cataract is a systemic disease such as human Down's syndrome, Hallerman-Streiff syndrome, Lowe syndrome, galactosemia, Marfan syndrome, Trismoy 13-15, Alport syndrome, myotonic dystrophy, Fabry disease, hypoparathroidism or Conradi syndrome.

230. The method of claim 216, wherein the developmental abnormality comprises embryonic lethality or reduced viability.

231. The method of claim 216, wherein the cardiovascular, endothelial or angiogenic disorders are arterial diseases, such as diabetes mellitus; papilledema; optic atrophy; atherosclerosis; angina; myocardial infarctions such as acute myocardial infarctions, cardiac hypertrophy, and heart failure such as congestive heart failure; hypertension; inflammatory vasculitides; Reynaud's disease and Reynaud's phenomenon; aneurysms and arterial restenosis; venous and lymphatic disorders such as thrombophlebitis, lymphangitis, and lymphedema; peripheral vascular disease; cancer such as vascular tumors, e.g., hemangioma (capillary and cavernous), glomus tumors, telangiectasia, bacillary angiomatosis, hemangioendothelioma, angiosarcoma, haemangiopericytoma, Kaposi's sarcoma, lymphangioma, and lymphangiosarcoma; tumor angiogenesis; trauma such as wounds, burns, and other injured tissue, implant fixation, scarring; ischemia reperfusion injury; rheumatoid arthritis; cerebrovascular disease; renal diseases such as acute renal failure, or osteoporosis.

232. The method of claim 216, wherein the immunological disorders are systemic lupus erythematosis; rheumatoid arthritis; juvenile chronic arthritis; spondyloarthropathies; systemic sclerosis (scleroderma); idiopathic inflammatory myopathies (dermatomyositis, polymyositis); Sjögren's syndrome; systemic vasculitis; sarcoidosis; autoimmune hemolytic anemia (immune pancytopenia, paroxysmal nocturnal hemoglobinuria); autoimmune thrombocytopenia (idiopathic thrombocytopenic purpura, immune-mediated thrombocytopenia); thyroiditis (Grave's disease, Hashimoto's thyroiditis, juvenile lymphocytic thyroiditis, atrophic thyroiditis); diabetes mellitus; immune-mediated renal disease (glomerulonephritis, tubulointerstitial nephritis); demyelinating diseases of the central and peripheral nervous systems such as multiple sclerosis, idiopathic demyelinating polyneuropathy or Guillain-Barré syndrome, and chronic inflammatory demyelinating polyneuropathy; hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis; inflammatory bowel disease (ulcerative colitis: Crohn's disease); gluten-sensitive enteropathy, and Whipple's disease; autoimmune or immune-mediated skin diseases including bullous skin diseases, erythema multiforme and contact dermatitis, psoriasis; allergic diseases such as asthma, allergic rhinitis, atopic dermatitis, food hypersensitivity and urticaria; immunologic diseases of the lung such as eosinophilic pneumonia, idiopathic pulmonary fibrosis and hypersensitivity pneumonitis; or transplantation associated diseases including graft rejection and graft-versus-host disease.

233. The method of claim 216, wherein said bone metabolic abnormality or disorder is arthritis, osteoporosis or osteopetrosis.

234. The method of claim 216, wherein the non-human transgenic animal exhibits at least one of the following physiological characteristics compared with gender matched wild-type littermates: increased anxiety-like response during open field testing; decreased anxiety-like response during open field activity testing; hyperactivity with increased rearing and hole poke activity during open field testing; hypoactivity with decreased rearing and hole poke activity during open field testing; increased exploratory activity during open-field testing; decreased exploratory activity during open-field testing; augmentation of circadian rhythm; abnormal circadian rhythm during home-cage activity testing including decreased ambulatory counts; abnormal circadian rhythm during home-cage activity testing including increased ambulatory counts; enhanced circadian rhythm; increased stress induced hyperthermia with increased stress response; increased resistance to stress induced hyperthermia; decreased resistance to stress induced hyperthermia; impaired motor coordination during inverted screen testing; increased depressive-like response during tail suspension testing; decreased depressive-like response during tail suspension testing; decreased startle response during prepulse inhibition testing; no startle response indicating deafness; reduced latency to respond in hot plate testing; increased pain perception in hot plate testing; prolonged latency to respond in hot plate testing; decreased pain perception in hot plate testing; straub tails during functional observational battery testing; opthamological abnormalities; attenuated retinal arteries; optic nerve abnormalities; retinal degeneration; retinal depigmentation; cataracts; decreased heart rate; decreased mean systolic blood pressure; increased mean systolic blood pressure; increased insulin sensitivity; increased mean fasting serum glucose levels; decreased mean serum glucose levels; increased mean serum cholesterol levels; decreased mean serum cholesterol levels; increased mean serum triglyceride levels; decreased mean serum triglyceride levels; enhanced glucose tolerance; impaired glucose tolerance; decreased mean serum insulin levels; increased uric acid levels; ketonemia; increased mean serum phosphorous levels; increased mean serum potassium levels; increased mean serum alkaline phosphatase levels; decreased mean serum alkaline phosphatase levels; blood in the urine; increased nitrituria; ketonuria; decreased mean serum albumin; decreased mean percentage of natural killer cells; abnormal leukocyte count; increased mean percentage of CD4 cells; decreased mean percentage of CD4 cells; increased mean percentage of B cells in peripheral blood; increase in CD4+ and CD8+ cells with decrease in B cells; decreased B cells and less CD11 blow cells in peritoneum; increased mean percentage B cells in spleen, lymph nodes and Peyer's patches; increase in activated/memory T cells by CD25+ staining and CD62L/CD44 staining; increase in activated/memory T cells in spleen; decreased mean percentage of CD8+ cells; increase total white blood cells (increase in neutrophils, lymphocytes, monocytes and basophils); decreased lymphocytes; increased mean absolute monocyte count; increased mean absolute neutrophil count; decreased mean absolute monocyte count; decreased mean serum IgM, IgA, IgG3, IgG2b and IgG2a levels; decreased mean serum IgG3 levels; decreased mean serum IgM levels; decreased mean serum IgG2a levels; decreased mean serum IgG3 and IgM levels; increase in mean serum IgM levels; increase in mean serum IgG2a levels; increase in mean serum IgG2b levels; anemia; decreased red blood cell count, decreased hemoglobin and decreased hematocrit; increased mean corpuscular volume; increased mean corpuscular hemoglobin; decreased mean corpuscular volume; decreased mean corpuscular hemoglobin; increased red blood cell distribution width and mean platelet volume; decreased red blood cell distribution width; skewed ratios of B220med/CD23− and B220+/CD11− low/CD23− cells after peritoneal lavage; increased CD25 T cells in lymph node and spleen; increased CD38 non-lymphoid cells in Peyer's patches; increased CD23 B cells (peritoneal); decreased percentage of CD4/CD8 DP cells and increased percentage of TCRB+ cells in thymus; decrease in Peyer's patch B cells; reduced number of TCRB+ CD38+ activated T cells in Peyer's patches; increased splenic CD25+ cells and peritoneal CD23 B cells; increased mean platelet count; decreased mean platelet count; decreased mean serum IgG response to an ovalbumin challenge; decreased mean serum IgG2a response to an ovalbumin challenge; increased mean serum IgG2a response to an ovalbumin challenge; increased mean serum MCP-1 response to a LPS challenge; increased mean serum TNF-alpha response to a LPS challenge; increased mean serum IL-6 response to a LPS challenge; increased skin fibroblast proliferation; decreased skin fibroblast proliferation; increased mean percent of total body fat and total fat mass; increased mean body weight; increased mean body length; increased total tissue mass (TTM); increased lean body mass (LBM); increased femoral bone mineral density (BMD); increased vertebral bone mineral density (BMD); increased BMC/LBM ratio; increased bone mineral density (BMD); increased total body volumetric bone mineral density (vBMD); increased bone mineral content (BMC); increased mean femoral midshaft cortical thickness and cross-sectional area; increased mean vertebral trabecular bone volume, number and connectivity density; decreased mean percent of total body fat and total fat mass; decreased mean body weight; decreased mean body length; decreased total tissue mass (TTM); decreased lean body mass (LBM); decreased femoral bone mineral density (BMD); decreased vertebral bone mineral density (BMD); decreased BMC/LBM ratio; decreased bone mineral density (BMD); decreased bone mineral content (BMC); decreased volumetric bone mineral density (vBMD); decreased mean femoral midshaft cortical thickness and cross-sectional area; decreased mean vertebral trabecular bone volume, number and connectivity density; myeloid hyperplasia in bone marrow; osteopetrosis with increased bone mineralization; increase in abdominal fat depots; chronic-active arthritis; proliferative chondrapathy and arthropathy; proliferation of cartilage in femoral tibia joints; chondrous metaplasia of cruciate ligaments and perichondral connective tissues; chronic active dermatitis; chronic active inflammation in periarticular tissues; chronic inflammation in various tissues; myeloid hyperplasia in femur and sternum with associated erythroid hyperplasia in the spleen; increased spleen weight; impaired gastrointestinal motility; thymic atrophy; thymic T cell lymphoma; growth retardation; development abnormalities; stunted growth with general reduction in all organ size; growth retardation with reduced viability; and embryonic lethality.

235. An agent identified by the method of claim 216.

236. The agent of claim 235 which is an agonist or antagonist of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide.

237. The agent of claim 236, wherein the agonist is an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody.

238. The agent of claim 236, wherein the antagonist is an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody.

239. A therapeutic agent identified by the method of claim 216.

240. A method of identifying an agent that modulates the expression of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising:
(a) contacting a test agent with a host cell expressing a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide; and
(b) determining whether the test agent modulates the expression of the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide by the host cell.

241. An agent identified by the method of claim 240.

242. The agent of claim 241 which is an agonist or antagonist of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide.

243. The agent of claim 242, wherein the agonist is an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody.

244. The agent of claim 242, wherein the antagonist is an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody.

245. A method of evaluating a therapeutic agent capable of affecting a condition associated with a disruption of a gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising:
(a) providing a non-human transgenic animal whose genome comprises a disruption of a gene which is an ortholog of a human gene that encodes for the PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide;
(b) measuring a physiological characteristic of the non-human transgenic animal of (a);
(c) comparing the measured physiological characteristic of (b) with that of a gender matched wild-type animal, wherein the physiological characteristic of the non-human transgenic animal that differs from the physiological characteristic of the wild-type animal is identified as a condition resulting from the gene disruption in the non-human transgenic animal;
(d) administering a test agent to the non-human transgenic animal of (a); and
(e) evaluating the effects of the test agent on the identified condition associated with gene disruption in the non-human transgenic animal.

246. The method of claim 245, wherein the condition is a neurological disorder; a cardiovascular, endothelial or angiogenic disorder; an eye abnormality; an immunological disorder; an oncological disorder; a bone metabolic abnormality or disorder; a lipid metabolic disorder; or a developmental abnormality.

247. A therapeutic agent identified by the method of claim 245.

248. The therapeutic agent of claim 247 which is an agonist or antagonist of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide.

249. The therapeutic agent of claim 248, wherein the agonist is an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody.

250. The therapeutic agent of claim 248, wherein the antagonist is an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody.

251. A pharmaceutical composition comprising the therapeutic agent of claim 247.

252. A method of treating or preventing or ameliorating a neurological disorder; cardiovascular, endothelial or angiogenic disorder; immunological disorder; oncological disorder; bone metabolic abnormality or disorder, or embryonic lethality associated with the disruption of a gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising administering to a subject in need of such treatment whom may already have the disorder, or may be prone to have the disorder or may be in whom the disorder is to be prevented, a therapeutically effective amount of the therapeutic agent of claim 239, or agonists or antagonists thereof, thereby effectively treating or preventing or ameliorating said disorder.

253. The method of claim 252, wherein the neurological disorder is an increased anxiety-like response during open field activity testing.

254. The method of claim 252, wherein the neurological disorder is a decreased anxiety-like response during open field activity testing.

255. The method of claim 252, wherein the neurological disorder is an abnormal circadian rhythm during home-cage activity testing.

256. The method of claim 252, wherein the neurological disorder is an enhanced motor coordination during inverted screen testing.

257. The method of claim 252, wherein the neurological disorder is an impaired motor coordination during inverted screen testing.

258. The method of claim 252, wherein the neurological disorder is depression, generalized anxiety disorders, attention deficit disorder, sleep disorder, hyperactivity disorder, obsessive compulsive disorder, schizophrenia, cognitive disorders, hyperalgesia or sensory disorders.

259. The method of claim 252, wherein the eye abnormality is a retinal abnormality.

260. The method of claim 252, wherein the eye abnormality is consistent with vision problems or blindness.

261. The method of claim 259, wherein the retinal abnormality is consistent with retinitis pigmentosa.

262. The method of claim 259, wherein the retinal abnormality is characterized by retinal degeneration or retinal dysplasia.

263. The method of claim 259, wherein the retinal abnormality is consistent with retinal dysplasia, various retinopathies, including retinopathy of prematurity, retrolental fibroplasia, neovascular glaucoma, age-related macular degeneration, diabetic macular edema, corneal neovascularization, corneal graft neovascularization, corneal graft rejection, retinal/choroidal neovascularization, neovascularization of the angle (rubeosis), ocular neovascular disease, vascular restenosis, arteriovenous malformations (AVM), meningioma, hemangioma, angiofibroma, thyroid hyperplasias (including Grave's disease), corneal and other tissue transplantation, retinal artery obstruction or occlusion; retinal degeneration causing secondary atrophy of the retinal vasculature, retinitis pigmentosa, macular dystrophies, Stargardt's disease, congenital stationary night blindness, choroideremia, gyrate atrophy, Leber's congenital amaurosis, retinoschisis disorders, Wagner's syndrome, Usher syndromes, Zellweger syndrome, Saldino-Mainzer syndrome, Senior-Loken syndrome, Bardet-Biedl syndrome, Alport's syndrome, Alstrom's syndrome, Cockayne's syndrome, dysplaisa spondyloepiphysaria congentia, Flynn-Aird syndrome, Friedreich ataxia, Hallgren syndrome, Marshall syndrome, Albers-Schnoberg disease, Refsum's disease, Kearns-Sayre syndrome, Waardenburg's syndrome, Alagile syndrome, myotonic dystrophy, olivopontocerebellar atrophy, Pierre-Marie dunsdrome, Stickler syndrome, carotinemeia, cystinosis, Wolfram syndrome, Bassen-Kornzweig syndrome, abetalipoproteinemia, incontinentia pigmenti, Batten's disease, mucopolysaccharidoses, homocystinuria, or mannosidosis.

264. The method of claim 252, wherein the eye abnormality is a cataract.

265. The method of claim 264, wherein the cataract is a systemic disease such as human Down's syndrome, Hallerman-Streiff syndrome, Lowe syndrome, galactosemia, Marfan syndrome, Trismoy 13-15, Alport syndrome, myotonic dystrophy, Fabry disease, hypoparathroidism or Conradi syndrome.

266. The method of claim 252, wherein the developmental abnormality comprises embryonic lethality or reduced viability.

267. The method of claim 252, wherein the cardiovascular, endothelial or angiogenic disorders are arterial diseases, such as diabetes mellitus; papilledema; optic atrophy; atherosclerosis; angina; myocardial infarctions such as acute myocardial infarctions, cardiac hypertrophy, and heart failure such as congestive heart failure; hypertension; inflammatory vasculitides; Reynaud's disease and Reynaud's phenomenon; aneurysms and arterial restenosis; venous and lymphatic disorders such as thrombophlebitis, lymphangitis, and lymphedema; peripheral vascular disease; cancer such as vascular tumors, e.g., hemangioma (capillary and cavernous), glomus tumors, telangiectasia, bacillary angiomatosis, hemangioendothelioma, angiosarcoma, haemangiopericytoma, Kaposi's sarcoma, lymphangioma, and lymphangiosarcoma; tumor angiogenesis; trauma such as wounds, burns, and other injured tissue, implant fixation, scarring; ischemia reperfusion injury; rheumatoid arthritis; cerebrovascular disease; renal diseases such as acute renal failure, or osteoporosis.

268. The method of claim 252, wherein the immunological disorders are systemic lupus erythematosis; rheumatoid arthritis; juvenile chronic arthritis spondyloarthropathies; systemic sclerosis (scleroderma); idiopathic inflammatory myopathies (dermatomyositis, polymyositis); Sjögren's syndrome; systemic vasculitis; sarcoidosis; autoimmune hemolytic anemia (immune pancytopenia, paroxysmal nocturnal hemoglobinuria); autoimmune thrombocytopenia (idiopathic thrombocytopenic purpura, immune-mediated thrombocytopenia); thyroiditis (Grave's disease, Hashimoto's thyroiditis, juvenile lymphocytic thyroiditis, atrophic thyroiditis); diabetes mellitus; immune-mediated renal disease (glomerulonephritis, tubulointerstitial nephritis); demyelinating diseases of the central and peripheral nervous systems such as multiple sclerosis, idiopathic demyelinating polyneuropathy or Guillain-Barré syndrome, and chronic inflammatory demyelinating polyneuropathy; hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis; inflammatory bowel disease (ulcerative colitis: Crohn's disease); gluten-sensitive enteropathy, and Whipple's disease; autoimmune or immune-mediated skin diseases including bullous skin diseases, erythema multiforme and contact dermatitis, psoriasis; allergic diseases such as asthma, allergic rhinitis, atopic dermatitis, food hypersensitivity and urticaria; immunologic diseases of the lung such as eosinophilic pneumonia, idiopathic pulmonary fibrosis and hypersensitivity pneumonitis; or transplantation associated diseases including graft rejection and graft-versus-host disease.

269. The method of claim 252, wherein said bone metabolic abnormality or disorder is arthritis, osteoporosis or osteopetrosis.

270. A method of modulating a phenotype associated with a disruption of a gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising administering to a subject whom may already have the phenotype, or may be prone to have the phenotype or may be in whom the phenotype is to be prevented, an effective amount of the agent of claim 195, or agonists or antagonists thereof, thereby effectively modulating the phenotype.

271. A method of modulating a physiological characteristic associated with a disruption of a gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising administering to a subject whom may already exhibit the physiological characteristic, or may be prone to exhibit the physiological characteristic or may be in whom the physiological characteristic is to be prevented, an effective amount of the agent of claim 201, or agonists or antagonists thereof, thereby effectively modulating the physiological characteristic.

272. A method of modulating a behavior associated with a disruption of a gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising administering to a subject whom may already exhibit the behavior, or may be prone to exhibit the behavior or may be in whom the exhibited behavior is to be prevented, an effective amount of the agent of claim 212, or agonists or antagonists thereof, thereby effectively modulating the behavior.

273. A method of modulating the expression of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising administering to a host cell expressing said PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, an effective amount of the agent of claim 241, or agonists or antagonists thereof, thereby effectively modulating the expression of said polypeptide.

274. A method of modulating a condition associated with a disruption of a gene which encodes for a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising administering to a subject whom may have the condition, or may be prone to have the condition or may be in whom the condition is to be prevented, a therapeutically effective amount of the therapeutic agent of claim 247, or agonists or antagonists thereof, thereby effectively modulating the condition.

275. A method of identifying an agent that mimics a condition or phenotype associated with a disruption in a gene which encodes a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising:
(a) providing a non-human transgenic animal whose genome comprises a disruption of a gene which is an ortholog of a human gene that encodes a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide;
(b) measuring a physiological characteristic of the non-human transgenic animal of (a);
(c) comparing the measured physiological characteristic of (b) with that of a gender matched wild-type animal, wherein the physiological characteristic of the non-human transgenic animal that differs from the physiological characteristic of the gender matched wild-type animal is identified as a condition or phenotype resulting from the gene disruption in the non-human transgenic animal;
(d) administering a test agent to said gender matched wild-type animal; and
(e) determining whether said test agent mimics the condition or phenotype initially observed in the non-human transgenic animal.

276. The method of claim 275, wherein the condition or phenotype associated with the disruption of the gene which is an ortholog of a human gene that encodes a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide is enhanced glucose tolerance.

277. The method of claim 275, wherein the condition or phenotype associated with the disruption of the gene which is an ortholog of a human gene that encodes a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide is increased insulin sensitivity.

278. An agent identified by the method of claim 275.

279. The agent of claim 278 which is an antagonist of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide.

280. The agent of claim 279, wherein the antagonist is an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody.

281. A method of mimicking a condition or phenotype associated with a disruption of a gene which encodes a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising administering to a subject in whom the condition or phenotype is to be mimicked, an effective amount of the agent of claim 278 or an antagonist of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, thereby effectively mimicking the condition or phenotype.

282. The method of claim 281, wherein the condition or phenotype associated with the disruption of the gene which is an ortholog of a human gene that encodes a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide is enhanced glucose tolerance.

283. The method of claim 281, wherein the condition or phenotype associated with the disruption of the gene which is an ortholog of a human gene that encodes a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide is increased insulin sensitivity.

284. A method of evaluating a therapeutic agent capable of mimicking a condition or phenotype associated with a disruption of a gene which encodes a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising:
(a) providing a non-human transgenic animal whose genome comprises a disruption of a gene which is an ortholog of a human gene that encodes a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide;
(b) measuring a physiological characteristic of the non-human transgenic animal of (a);
(c) comparing the measured physiological characteristic of (b) with that of a gender matched wild-type animal, wherein the physiological characteristic of the non-human transgenic animal that differs from the physiological characteristic of the gender matched wild-type animal is identified as a condition or phenotype resulting from the gene disruption in the non-human transgenic animal;
(d) administering a test agent to said gender matched wild-type animal of (c); and
(e) evaluating the ability of the test agent to mimic the condition or phenotype associated with gene disruption in the non-human transgenic animal.

285. A therapeutic agent identified by the method of claim 284.

286. The therapeutic agent of claim 285 which is an antagonist of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide.

287. The therapeutic agent of claim 286, wherein the antagonist is an anti-PRO179, anti-PRO181, anti-PRO244, anti-PRO247, anti-PRO269, anti-PRO293, anti-PRO298, anti-PRO339, anti-PRO341, anti-PRO347, anti-PRO531, anti-PRO537, anti-PRO718, anti-PRO773, anti-PRO860, anti-PRO871, anti-PRO872, anti-PRO813, anti-PRO828, anti-PRO1100, anti-PRO1114, anti-PRO1115, anti-PRO1126, anti-PRO1133, anti-PRO1154, anti-PRO1185, anti-PRO1194, anti-PRO1287, anti-PRO1291, anti-PRO1293, anti-PRO1310, anti-PRO1312, anti-PRO1335, anti-PRO1339, anti-PRO2155, anti-PRO1356, anti-PRO1385, anti-PRO1412, anti-PRO1487, anti-PRO1758, anti-PRO1779, anti-PRO1785, anti-PRO1889, anti-PRO90318, anti-PRO3434, anti-PRO3579, anti-PRO4322, anti-PRO4343, anti-PRO4347, anti-PRO4403, anti-PRO4976, anti-PRO260, anti-PRO6014, anti-PRO6027, anti-PRO6181, anti-PRO6714, anti-PRO9922, anti-PRO7179, anti-PRO7476, anti-PRO9824, anti-PRO19814, anti-PRO19836, anti-PRO20088, anti-PRO70789, anti-PRO50298, anti-PRO51592, anti-PRO1757, anti-PRO4421, anti-PRO9903, anti-PRO1106, anti-PRO1411, anti-PRO1486, anti-PRO1565, anti-PRO4399 or anti-PRO4404 antibody.

288. A pharmaceutical composition comprising the therapeutic agent of claim 285.

289. A method of mimicking a condition or phenotype associated with a disruption of a gene which encodes a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, the method comprising administering to a subject in whom the condition or phenotype disorder is to be mimicked, a therapeutically effective amount of the therapeutic agent of claim 285, or an antagonist of a PRO179, PRO181, PRO244, PRO247, PRO269, PRO293, PRO298, PRO339, PRO341, PRO347, PRO531, PRO537, PRO718, PRO773, PRO860, PRO871, PRO872, PRO813, PRO828, PRO1100, PRO1114, PRO1115, PRO1126, PRO1133, PRO1154, PRO1185, PRO1194, PRO1287, PRO1291, PRO1293, PRO1310, PRO1312, PRO1335, PRO1339, PRO2155, PRO1356, PRO1385, PRO1412, PRO1487, PRO1758, PRO1779, PRO1785, PRO1889, PRO90318, PRO3434, PRO3579, PRO4322, PRO4343, PRO4347, PRO4403, PRO4976, PRO260, PRO6014, PRO6027, PRO6181, PRO6714, PRO9922, PRO7179, PRO7476, PRO9824, PRO19814, PRO19836, PRO20088, PRO70789, PRO50298, PRO51592, PRO1757, PRO4421, PRO9903, PRO1106, PRO1411, PRO1486, PRO1565, PRO4399 or PRO4404 polypeptide, thereby effectively mimicking the condition or phenotype.


SEQUENCE LISTING (266 sequences)

SEQ ID NO: 1 - DNA, 2042 bp, Homo sapiens
gcggacgcgtgggtgaaattgaaaatcaagataaaaatgttcacaattaagctccttctttttattgttcctctagttatttcctccagaattgatcaagacaattcatcatttgattctctatctccagagccaaaatcaagatttgctatgttagacgatgtaaaaattttagccaatggcctccttcagttgggacatggtcttaaagactttgtccataagacgaagggccaaattaatgacatatttcaaaaactcaacatatttgatcagtctttttatgatctatcgctgcaaaccagtgaaatcaaagaagaagaaaaggaactgagaagaactacatataaactacaagtcaaaaatgaagaggtaaagaatatgtcacttgaactcaactcaaaacttgaaagcctcctagaagaaaaaattctacttcaacaaaaagtgaaatatttagaagagcaactaactaacttaattcaaaatcaacctgaaactccagaacacccagaagtaacttcacttaaaacttttgtagaaaaacaagataatagcatcaaagaccttctccagaccgtggaagaccaatataaacaattaaaccaacagcatagtcaaataaaagaaatagaaaatcagctcagaaggactagtattcaagaacccacagaaatttctctatcttccaagccaagagcaccaagaactactccctttcttcagttgaatgaaataagaaatgtaaaacatgatggcattcctgctgaatgtaccaccatttataacagaggtgaacatacaagtggcatgtatgccatcagacccagcaactctcaagtttttcatgtctactgtgatgttatatcaggtagtccatggacattaattcaacatcgaatagatggatcacaaaacttcaatgaaacgtgggagaactacaaatatggttttgggaggcttgatggagaattttggttgggcctagagaagatatactccatagtgaagcaatctaattatgttttacgaattgagttggaagactggaaagacaacaaacattatattgaatattctttttacttgggaaatcacgaaaccaactatacgctacatctagttgcgattactggcaatgtccccaatgcaatcccggaaaacaaagatttggtgttttctacttgggatcacaaagcaaaaggacacttcaactgtccagagggttattcaggaggctggtggtggcatgatgagtgtggagaaaacaacctaaatggtaaatataacaaaccaagagcaaaatctaagccagagaggagaagaggattatcttggaagtctcaaaatggaaggttatactctataaaatcaaccaaaatgttgatccatccaacagattcagaaagctttgaatgaactgaggcaatttaaaggcatatttaaccattaactcattccaagttaatgtggtctaataatctggtataaatccttaagagaaagcttgagaaatagattttttttatcttaaagtcactgtctatttaagattaaacatacaatcacataaccttaaagaataccgtttacatttctcaatcaaaattcttataatactatttgttttaaattttgtgatgtgggaatcaattttagatggtcacaatctagattataatcaataggtgaacttattaaataacttttctaaataaaaaatttagagacttttattttaaaaggcatcatatgagctaatatcacaactttcccagtttaaaaaactagtactcttgttaaaactctaaacttgactaaatacagaggactggtaattgtacagttcttaaatgttgtagtattaatttcaaaactaaaaatcgtcagcacagagtatgtgtaaaaatctgtaatacaaatttttaaactgatgcttcattttgctacaaaataatttggagtaaatgtttgatatgatttatttatgaaacctaatgaagcagaattaaatactgtattaaaataagttcgctgtcttt

SEQ ID NO: 2 - protein, 460 aa, Homo sapiens
MFTIKLLLFIVPLVISSRIDQDNSSFDSLSPEPKSRFAMLDDVKILANGLLQLGHGLKDFVHKTKGQINDIFQKLNIFDQSFYDLSLQTSEIKEEEKELRRTTYKLQVKNEEVKNMSLELNSKLESLLEEKILLQQKVKYLEEQLTNLIQNQPETPEHPEVTSLKTFVEKQDNSIKDLLQTVEDQYKQLNQQHSQIKEIENQLRRTSIQEPTEISLSSKPRAPRTTPFLQLNEIRNVKHDGIPAECTTIYNRGEHTSGMYAIRPSNSQVFHVYCDVISGSPWTLIQHRIDGSQNFNETWENYKYGFGRLDGEFWLGLEKIYSIVKQSNYVLRIELEDWKDNKHYIEYSFYLGNHETNYTLHLVAITGNVPNAIPENKDLVFSTWDHKAKGHFNCPEGYSGGWWWHDECGENNLNGKYNKPRAKSKPERRRGLSWKSQNGRLYSIKSTKMLIHPTDSESFE

SEQ ID NO: 3 - DNA, 1333 bp, Homo sapiens
gcccacgcgtccgatggcgttcacgttcgcggccttctgctacatgctggcgctgctgctcactgccgcgctcatcttcttcgccatttggcacattatagcatttgatgagctgaagactgattacaagaatcctatagaccagtgtaataccctgaatccccttgtactcccagagtacctcatccacgctttcttctgtgtcatgtttctttgtgcagcagagtggcttacactgggtctcaatatgcccctcttggcatatcatatttggaggtatatgagtagaccagtgatgagtggcccaggactctatgaccctacaaccatcatgaatgcagatattctagcatattgtcagaaggaaggatggtgcaaattagctttttatcttctagcatttttttactacctatatggcatgatctatgttttggtgagctcttagaacaacacacagaagaattggtccagttaagtgcatgcaaaaagccaccaaatgaagggattctatccagcaagatcctgtccaagagtagcctgtggaatctgatcagttactttaaaaaatgactccttattttttaaatgtttccacatttttgcttgtggaaagactgttttcatatgttatactcagataaagattttaaatggtattacgtataaattaatataaaatgattacctctggtgttgacaggtttgaacttgcacttcttaaggaacagccataatcctctgaatgatgcattaattactgactgtcctagtacattggaagcttttgtttataggaacttgtagggctcattttggtttcattgaaacagtatctaattataaattagctgtagatatcaggtgcttctgatgaagtgaaaatgtatatctgactagtgggaaacttcatgggtttcctcatctgtcatgtcgatgattatatatggatacatttacaaaaataaaaagcgggaattttcccttcgcttgaatattatccctgtatattgcatgaatgagagatttcccatatttccatcagagtaataaatatacttgctttaattcttaagcataagtaaacatgatataaaaatatatgctgaattacttgtgaagaatgcatttaaagctattttaaatgtgtttttatttgtaagacattacttattaagaaattggttattatgcttactgttctaatctggtggtaaaggtattcttaagaatttgcaggtactacagattttcaaaactgaatgagagaaaattgtataaccatcctgctgttcctttagtgcaatacaataaaactctgaaattaagactc

SEQ ID NO: 4 - protein, 144 aa, Homo sapiens
MAFTFAAFCYMLALLLTAALIFFAIWHIIAFDELKTDYKNPIDQCNTLNPLVLPEYLIHAFFCVMFLCAAEWLTLGLNMPLLAYHIWRYMSRPVMSGPGLYDPTTIMNADILAYCQKEGWCKLAFYLLAFFYYLYGMIYVLVSS

SEQ ID NO: 5 - DNA, 997 bp, Homo sapiens
cccacgcgtccgatcttaccaacaaaacactcctgaggagaaagaaagagagggagggagagaaaaagagagagagagaaacaaaaaaccaaagagagagaaaaaatgaattcatctaaatcatctgaaacacaatgcacagagagaggatgcttctcttcccaaatgttcttatggactgttgctgggatccccatcctatttctcagtgcctgtttcatcaccagatgtgttgtgacatttcgcatctttcaaacctgtgatgagaaaaagtttcagctacctgagaatttcacagagctctcctgctacaattatggatcaggttcagtcaagaattgttgtccattgaactgggaatattttcaatccagctgctacttcttttctactgacaccatttcctgggcgttaagtttaaagaactgctcagccatgggggctcacctggtggttatcaactcacaggaggagcaggaattcctttcctacaagaaacctaaaatgagagagttttttattggactgtcagaccaggttgtcgagggtcagtggcaatgggtggacggcacacctttgacaaagtctctgagcttctgggatgtaggggagcccaacaacatagctaccctggaggactgtgccaccatgagagactcttcaaacccaaggcaaaattggaatgatgtaacctgtttcctcaattattttcggatttgtgaaatggtaggaataaatcctttgaacaaaggaaaatctctttaagaacagaaggcacaactcaaatgtgtaaagaaggaagagcaagaacatggccacacccaccgccccacacgagaaatttgtgcgctgaacttcaaaggacttcataagtatttgttactctgatacaaataaaaataagtagttttaaatgttaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 6 - protein, 219 aa, Homo sapiens
MNSSKSSETQCTERGCFSSQMFLWTVAGIPILFLSACFITRCVVTFRIFQTCDEKKFQLPENFTELSCYNYGSGSVKNCCPLNWEYFQSSCYFFSTDTISWALSLKNCSAMGAHLVVINSQEEQEFLSYKKPKMREFFIGLSDQVVEGQWQWVDGTPLTKSLSFWDVGEPNNIATLEDCATMRDSSNPRQNWNDVTCFLNYFRICEMVGINPLNKGKSL

SEQ ID NO: 7 - DNA, 3401 bp, Homo sapiens
gcaagccaaggcgctgtttgagaaggtgaagaagttccggacccatgtggaggagggggacattgtgtaccgcctctacatgcggcagaccatcatcaaggtgatcaagttcatcctcatcatctgctacaccgtctactacgtgcacaacatcaagttcgacgtggactgcaccgtggacattgagagcctgacgggctaccgcacctaccgctgtgcccaccccctggccacactcttcaagatcctggcgtccttctacatcagcctagtcatcttctacggcctcatctgcatgtacacactgtggtggatgctacggcgctccctcaagaagtactcgtttgagtcgatccgtgaggagagcagctacagcgacatccccgacgtcaagaacgacttcgccttcatgctgcacctcattgaccaatacgacccgctctactccaagcgcttcgccgtcttcctgtcggaggtgagtgagaacaagctgcggcagctgaacctcaacaacgagtggacgctggacaagctccggcagcggctcaccaagaacgcgcaggacaagctggagctgcacctgttcatgctcagtggcatccctgacactgtgtttgacctggtggagctggaggtcctcaagctggagctgatccccgacgtgaccatcccgcccagcattgcccagctcacgggcctcaaggagctgtggctctaccacacagcggccaagattgaagcgcctgcgctggccttcctgcgcgagaacctgcgggcgctgcacatcaagttcaccgacatcaaggagatcccgctgtggatctatagcctgaagacactggaggagctgcacctgacgggcaacctgagcgcggagaacaaccgctacatcgtcatcgacgggctgcgggagctcaaacgcctcaaggtgctgcggctcaagagcaacctaagcaagctgccacaggtggtcacagatgtgggcgtgcacctgcagaagctgtccatcaacaatgagggcaccaagctcatcgtcctcaacagcctcaagaagatggcgaacctgactgagctggagctgatccgctgcgacctggagcgcatcccccactccatcttcagcctccacaacctgcaggagattgacctcaaggacaacaacctcaagaccatcgaggagatcatcagcttccagcacctgcaccgcctcacctgccttaagctgtggtacaaccacatcgcctacatccccatccagatcggcaacctcaccaacctggagcgcctctacctgaaccgcaacaagatcgagaagatccccacccagctcttctactgccgcaagctgcgctacctggacctcagccacaacaacctgaccttcctccctgccgacatcggcctcctgcagaacctccagaacctagccatcacggccaaccggatcgagacgctccctccggagctcttccagtgccggaagctgcgggccctgcacctgggcaacaacgtgctgcagtcactgccctccagggtgggcgagctgaccaacctgacgcagatcgagctgcggggcaaccggctggagtgcctgcctgtggagctgggcgagtgcccactgctcaagcgcagcggcttggtggtggaggaggacctgttcaacacactgccacccgaggtgaaggagcggctgtggagggctgacaaggagcaggcctgagcgaggccggcccagcacagcaagcagcaggaccgctgcccagtcctcaggcccggaggggcaggcctagcttctcccagaactcccggacagccaggacagcctcgcggctgggcaggagcctggggccgcttgtgagtcaggccagagcgagaggacagtatctgtggggctggccccttttctccctctgagactcacgtcccccagggcaagtgcttgtggaggagagcaagtctcaagagcgcagtatttggataatcagggtctcctccctggaggccagctctgccccaggggctgagctgccaccagaggtcctgggaccctcactttagttcttggtatttatttttctccatctcccacctccttcatccagataacttatacattcccaagaaagttcagcccagatggaaggtgttcagggaaaggtgggctgccttttccccttgtccttatttagcgatgccgccgggcatttaacacccacctggacttcagcagagtggtccggggcgaaccagccatgggacggtcacccagcagtgccgggctgggctctgcggtgcggtccacgggagagcaggcctccagctggaaaggccaggcctggagcttgcctcttcagtttttgtggcagttttagttttttgttttttttttttttaatcaaaaaacaattttttttaaaaaaaagctttgaaaatggatggtttgggtattaaaaagaaaaaaaaaacttaaaaaaaaaaagacactaacggccagtgagttggagtctcagggcagggtggcagtttcccttgagcaaagcagccagacgttgaactgtgtttcctttccctgggcgcagggtgcagggtgtcttccggatctggtgtgaccttggtccaggagttctatttgttcctggggagggaggtttttttgtttgttttttgggtttttttggtgtcttgttttctttctcctccatgtgtcttggcaggcactcatttctgtggctgtcggccagagggaatgttctggagctgccaaggagggaggagactcgggttggctaatccccggatgaacggtgctccattcgcacctcccctcctcgtgcctgccctgcctctccacgcacagtgttaaggagccaagaggagccacttcgcccagactttgtttccccacctcctgcggcatgggtgtgtccagtgccaccgctggcctccgctgcttccatcagccctgtcgccacctggtccttcatgaagagcagacacttagaggctggtcgggaatggggaggtcgcccctgggagggcaggcgttggttccaagccggttcccgtccctggcgcctggagtgcacacagcccagtcggcacctggtggctggaagccaacctgctttagatcactcgggtccccaccttagaagggtccccgccttagatcaatcacgtggacactaaggcacgttttagagtctcttgtcttaatgattatgtccatccgtctgtccgtccatttgtgttttctgcgtcgtgtcattggatataatcctcagaaataatgcacactagcctctgacaaccatgaagcaaaaatccgttacatgtgggtctgaacttgtagactcggtcacagtatcaaataaaatctataacagaaaaaaaaaaaaaaa

SEQ ID NO: 8 - protein, 546 aa, Homo sapiens
MRQTIIKVIKFILIICYTVYYVHNIKFDVDCTVDIESLTGYRTYRCAHPLATLFKILASFYISLVIFYGLICMYTLWWMLRRSLKKYSFESIREESSYSDIPDVKNDFAFMLHLIDQYDPLYSKRFAVFLSEVSENKLRQLNLNNEWTLDKLRQRLTKNAQDKLELHLFMLSGIPDTVFDLVELEVLKLELIPDVTIPPSIAQLTGLKELWLYHTAAKIEAPALAFLRENLRALHIKFTDIKEIPLWIYSLKTLEELHLTGNLSAENNRYIVIDGLRELKRLKVLRLKSNLSKLPQVVTDVGVHLQKLSINNEGTKLIVLNSLKKMANLTELELIRCDLERIPHSIFSLHNLQEIDLKDNNLKTIEEIISFQHLHRLTCLKLWYNHIAYIPIQIGNLTNLERLYLNRNKIEKIPTQLFYCRKLRYLDLSHNNLTFLPADIGLLQNLQNLAITANRIETLPPELFQCRKLRALHLGNNVLQSLPSRVGELTNLTQIELRGNRLECLPVELGECPLLKRSGLVVEEDLFNTLPPEVKERLWRADKEQA

SEQ ID NO: 9 - DNA, 2226 bp, Homo sapiens
agtcgactgcgtcccctgtacccggcgccagctgtgttcctgaccccagaataactcagggctgcaccgggcctggcagcgctccgcacacatttcctgtcgcggcctaagggaaactgttggccgctgggcccgcggggggattcttggcagttggggggtccgtcgggagcgagggcggaggggaagggagggggaaccgggttggggaagccagctgtagagggcggtgaccgcgctccagacacagctctgcgtcctcgagcgggacagatccaagttgggagcagctctgcgtgcggggcctcagagaatgaggccggcgttcgccctgtgcctcctctggcaggcgctctggcccgggccgggcggcggcgaacaccccactgccgaccgtgctggctgctcggcctcgggggcctgctacagcctgcaccacgctaccatgaagcggcaggcggccgaggaggcctgcatcctgcgaggtggggcgctcagcaccgtgcgtgcgggcgccgagctgcgcgctgtgctcgcgctcctgcgggcaggcccagggcccggagggggctccaaagacctgctgttctgggtcgcactggagcgcaggcgttcccactgcaccctggagaacgagcctttgcggggtttctcctggctgtcctccgaccccggcggtctcgaaagcgacacgctgcagtgggtggaggagccccaacgctcctgcaccgcgcggagatgcgcggtactccaggccaccggtggggtcgagcccgcaggctggaaggagatgcgatgccacctgcgcgccaacggctacctgtgcaagtaccagtttgaggtcttgtgtcctgcgccgcgccccggggccgcctctaacttgagctatcgcgcgcccttccagctgcacagcgccgctctggacttcagtccacctgggaccgaggtgagtgcgctctgccggggacagctcccgatctcagttacttgcatcgcggacgaaatcggcgctcgctgggacaaactctcgggcgatgtgttgtgtccctgccccgggaggtacctccgtgctggcaaatgcgcagagctccctaactgcctagacgacttgggaggctttgcctgcgaatgtgctacgggcttcgagctggggaaggacggccgctcttgtgtgaccagtggggaaggacagccgacccttggggggaccggggtgcccaccaggcgcccgccggccactgcaaccagccccgtgccgcagagaacatggccaatcagggtcgacgagaagctgggagagacaccacttgtccctgaacaagacaattcagtaacatctattcctgagattcctcgatggggatcacagagcacgatgtctacccttcaaatgtcccttcaagccgagtcaaaggccactatcaccccatcagggagcgtgatttccaagtttaattctacgacttcctctgccactcctcaggctttcgactcctcctctgccgtggtcttcatatttgtgagcacagcagtagtagtgttggtgatcttgaccatgacagtactggggcttgtcaagctctgctttcacgaaagcccctcttcccagccaaggaaggagtctatgggcccgccgggcctggagagtgatcctgagcccgctgctttgggctccagttctgcacattgcacaaacaatggggtgaaagtcggggactgtgatctgcgggacagagcagagggtgccttgctggcggagtcccctcttggctctagtgatgcatagggaaacaggggacatgggcactcctgtgaacagtttttcacttttgatgaaacggggaaccaagaggaacttacttgtgtaactgacaatttctgcagaaatcccccttcctctaaattccctttactccactgaggagctaaatcagaactgcacactccttccctgatgatagaggaagtggaagtgcctttaggatggtgatactgggggaccgggtagtgctggggagagatattttcttatgtttattcggagaatttggagaagtgattgaacttttcaagacattggaaacaaatagaacacaatataatttacattaaaaaataatttctaccaaaatggaaaggaaatgttctatgttgttcaggctaggagtatattggttcgaaatcccagggaaaaaaataaaaataaaaaattaaaggattgttgat

SEQ ID NO: 10 - protein, 490 aa, Homo sapiens
MRPAFALCLLWQALWPGPGGGEHPTADRAGCSASGACYSLHHATMKRQAAEEACILRGGALSTVRAGAELRAVLALLRAGPGPGGGSKDLLFWVALERRRSHCTLENEPLRGFSWLSSDPGGLESDTLQWVEEPQRSCTARRCAVLQATGGVEPAGWKEMRCHLRANGYLCKYQFEVLCPAPRPGAASNLSYRAPFQLHSAALDFSPPGTEVSALCRGQLPISVTCIADEIGARWDKLSGDVLCPCPGRYLRAGKCAELPNCLDDLGGFACECATGFELGKDGRSCVTSGEGQPTLGGTGVPTRRPPATATSPVPQRTWPIRVDEKLGETPLVPEQDNSVTSIPEIPRWGSQSTMSTLQMSLQAESKATITPSGSVISKFNSTTSSATPQAFDSSSAVVFIFVSTAVVVLVILTMTVLGLVKLCFHESPSSQPRKESMGPPGLESDPEPAALGSSSAHCTNNGVKVGDCDLRDRAEGALLAESPLGSSDA

SEQ ID NO: 11 - DNA, 3679 bp, Homo sapiens
aaggaggctgggaggaaagaggtaagaaaggttagagaacctacctcacatctctctgggctcagaaggactctgaagataacaataatttcagcccatccactctccttccctcccaaacacacatgtgcatgtacacacacacatacacacacatacaccttcctctccttcactgaagactcacagtcactcactctgtgagcaggtcatagaaaaggacactaaagccttaaggacaggcctggccattacctctgcagctcctttggcttgttgagtcaaaaaacatgggaggggccaggcacggtgactcacacctgtaatcccagcattttgggagaccgaggtgagcagatcacttgaggtcaggagttcgagaccagcctggccaacatggagaaacccccatctctactaaaaatacaaaaattagccaggagtggtggcaggtgcctgtaatcccagctactcaggtggctgagccaggagaatcgcttgaatccaggaggcggaggatgcagtcagctgagtgcaccgctgcactccagcctgggtgacagaatgagactctgtctcaaacaaacaaacacgggaggaggggtagatactgcttctctgcaacctccttaactctgcatcctcttcttccagggctgcccctgatggggcctggcaatgactgagcaggcccagccccagaggacaaggaagagaaggcatattgaggagggcaagaagtgacgcccggtgtagaatgactgccctgggagggtggttccttgggccctggcagggttgctgacccttaccctgcaaaacacaaagagcaggactccagactctccttgtgaatggtcccctgccctgcagctccaccatgaggcttctcgtggccccactcttgctagcttgggtggctggtgccactgccactgtgcccgtggtaccctggcatgttccctgcccccctcagtgtgcctgccagatccggccctggtatacgccccgctcgtcctaccgcgaggctaccactgtggactgcaatgacctattcctgacggcagtccccccggcactccccgcaggcacacagaccctgctcctgcagagcaacagcattgtccgtgtggaccagagtgagctgggctacctggccaatctcacagagctggacctgtcccagaacagcttttcggatgcccgagactgtgatttccatgccctgccccagctgctgagcctgcacctagaggagaaccagctgacccggctggaggaccacagctttgcagggctggccagcctacaggaactctatctcaaccacaaccagctctaccgcatcgcccccagggccttttctggcctcagcaacttgctgcggctgcacctcaactccaacctcctgagggccattgacagccgctggtttgaaatgctgcccaacttggagatactcatgattggcggcaacaaggtagatgccatcctggacatgaacttccggcccctggccaacctgcgtagcctggtgctagcaggcatgaacctgcgggagatctccgactatgccctggaggggctgcaaagcctggagagcctctccttctatgacaaccagctggcccgggtgcccaggcgggcactggaacaggtgcccgggctcaagttcctagacctcaacaagaacccgctccagcgggtagggccgggggactttgccaacatgctgcaccttaaggagctgggactgaacaacatggaggagctggtctccatcgacaagtttgccctggtgaacctccccgagctgaccaagctggacatcaccaataacccacggctgtccttcatccacccccgcgccttccaccacctgccccagatggagaccctcatgctcaacaacaacgctctcagtgccttgcaccagcagacggtggagtccctgcccaacctgcaggaggtaggtctccacggcaaccccatccgctgtgactgtgtcatccgctgggccaatgccacgggcacccgtgtccgcttcatcgagccgcaatccaccctgtgtgcggagcctccggacctccagcgcctcccggtccgtgaggtgcccttccgggagatgacggaccactgtttgcccctcatctccccacgaagcttccccccaagcctccaggtagccagtggagagagcatggtgctgcattgccgggcactggccgaacccgaacccgagatctactgggtcactccagctgggcttcgactgacacctgcccatgcaggcaggaggtaccgggtgtaccccgaggggaccctggagctgcggagggtgacagcagaagaggcagggctatacacctgtgtggcccagaacctggtgggggctgacactaagacggttagtgtggttgtgggccgtgctctcctccagccaggcagggacgaaggacaggggctggagctccgggtgcaggagacccacccctatcacatcctgctatcttgggtcaccccacccaacacagtgtccaccaacctcacctggtccagtgcctcctccctccggggccagggggccacagctctggcccgcctgcctcggggaacccacagctacaacattacccgcctccttcaggccacggagtactgggcctgcctgcaagtggcctttgctgatgcccacacccagttggcttgtgtatgggccaggaccaaagaggccacttcttgccacagagccttaggggatcgtcctgggctcattgccatcctggctctcgctgtccttctcctggcagctgggctagcggcccaccttggcacaggccaacccaggaagggtgtgggtgggaggcggcctctccctccagcctgggctttctggggctggagtgccccttctgtccgggttgtgtctgctcccctcgtcctgccctggaatccagggaggaagctgcccagatcctcagaaggggagacactgttgccaccattgtctcaaaattcttgaagctcagcctgttctcagcagtagagaaatcactaggactactttttaccaaaagagaagcagtctgggccagatgccctgccaggaaagggacatggacccacgtgcttgaggcctggcagctgggccaagacagatggggctttgtggccctgggggtgcttctgcagccttgaaaaagttgcccttacctcctagggtcacctctgctgccattctgaggaacatctccaaggaacaggagggactttggctagagcctcctgcctccccatcttctctctgcccagaggctcctgggcctggcttggctgtcccctacctgtgtccccgggctgcaccccttcctcttctctttctctgtacagtctcagttgcttgctcttgtgcctcctgggcaagggctgaaggaggccactccatctcacctcggggggctgccctcaatgtgggagtgaccccagccagatctgaaggacatttgggagagggatgcccaggaacgcctcatctcagcagcctgggctcggcattccgaagctgactttctataggcaattttgtacctttgtggagaaatgtgtcacctcccccaacccgattcactcttttctcctgttttgtaaaaaataaaaataaataataacaataaaaaaa

SEQ ID NO: 12 - protein, 713 aa, Homo sapiens [sequence omitted]

SEQ ID NO: 13 - DNA, 2690 bp, Homo sapiens
  Unsure 2039-2065: Unknown base
ggttgccacagctggtttagggccccgaccactggggccccttgtcaggaggagacagcctcccggcccggggaggacaagtcgctgccacctttggctgccgacgtgattccctgggacggtccgtttcctgccgtcagctgccggccgagttgggtctccgtgtttcaggccggctcccccttcctggtctcccttctcccgctgggccggtttatcgggaggagattgtcttccagggctagcaattggacttttgatgatgtttgacccagcggcaggaatagcaggcaacgtgatttcaaagctgggctcagcctctgtttcttctctcgtgtaatcgcaaaacccattttggagcaggaattccaatcatgtctgtgatggtggtgagaaagaaggtgacacggaaatgggagaaactcccaggcaggaacaccttttgctgtgatggccgcgtcatgatggcccggcaaaagggcattttctacctgacccttttcctcatcctggggacatgtacactcttcttcgcctttgagtgccgctacctggctgttcagctgtctcctgccatccctgtatttgctgccatgctcttccttttctccatggctacactgttgaggaccagcttcagtgaccctggagtgattcctcgggcgctaccagatgaagcagctttcatagaaatggagatagaagctaccaatggtgcggtgccccagggccagcgaccaccgcctcgtatcaagaatttccagataaacaaccagattgtgaaactgaaatactgttacacatgcaagatcttccggcctccccgggcctcccattgcagcatctgtgacaactgtgtggagcgcttcgaccatcactgcccctgggtggggaattgtgttggaaagaggaactaccgctacttctacctcttcatcctttctctctccctcctcacaatctatgtcttcgccttcaacatcgtctatgtggccctcaaatctttgaaaattggcttcttggagacattgaaagaaactcctggaactgttctagaagtcctcatttgcttctttacactctggtccgtcgtgggactgactggatttcatactttcctcgtggctctcaaccagacaaccaatgaagacatcaaaggatcatggacagggaagaatcgcgtccagaatccctacagccatggcaatattgtgaagaactgctgtgaagtgctgtgtggccccttgccccccagtgtgctggatcgaaggggtattttgccactggaggaaagtggaagtcgacctcccagtactcaagagaccagtagcagcctcttgccacagagcccagcccccacagaacacctgaactcaaatgagatgccggaggacagcagcactcccgaagagatgccacctccagagcccccagagccaccacaggaggcagctgaagctgagaagtagcctatctatggaagagacttttgtttgtgtttaattagggctatgagagatttcaggtgagaagttaaacctgagacagagagcaagtaagctgtcccttttaactgtttttctttggtctttagtcacccagttgcacactggcattttcttgctgcaagcttttttaaatttctgaactcaaggcagtggcagaagatgtcagtcacctctgataactggaaaaatgggtctcttgggccctggcactggttctccatggcctcagccacagggtccccttggaccccctctcttccctccagatcccagccctcctgcttggggtcactggtctcattctggggctaaaagtttttgagactggctcaaatcctcccaagctgctgcacgtgctgagtccagaggcagtcacagagacctctggccaggggatcctaactgggttcttggggtcttcaggactgaagaggagggagagtggggtcagaagattctcctggccaccaagtgccagcattgcccacaaatccttttaggaatgggacaggtaccttccacttgttgtannnnnnnnnnnnnnnnnnnnnnnnnnnttgtttttccttttgactcctgctcccattaggagcaggaatggcagtaataaaagtctgcactttggtcatttcttttcctcagaggaagcccgagtgctcacttaaacactatcccctcagactccctgtgtgaggcctgcagaggccctgaatgcacaaatgggaaaccaaggcacagagaggctctcctctcctctcctctcccccgatgtaccctcaaaaaaaaaaaaatgctaaccagttcttccattaagcctcggctgagtgagggaaagcccagcactgctgccctctcgggtaactcaccctaaggcctcggcccacctctggctatggtaaccacactgggggcttcctccaagccccgctcttccagcacttccaccggcagagtcccagagccacttcaccctgggggtgggctgtggcccccagtcagctctgctcaggacctgctctatttcagggaagaagatttatgtattatatgtggctatatttcctagagcacctgtgttttcctctttctaagccagggtcctgtctggatgacttatgcggtgggggagtgtaaaccggaacttttcatctatttgaaggcgattaaactgtgtctaatgca

SEQ ID NO: 14 - protein, 364 aa, Homo sapiens
MSVMVVRKKVTRKWEKLPGRNTFCCDGRVMMARQKGIFYLTLFLILGTCTLFFAFECRYLAVQLSPAIPVFAAMLFLFSMATLLRTSFSDPGVIPRALPDEAAFIEMEIEATNGAVPQGQRPPPRIKNFQINNQIVKLKYCYTCKIFRPPRASHCSICDNCVERFDHHCPWVGNCVGKRNYRYFYLFILSLSLLTIYVFAFNIVYVALKSLKIGFLETLKETPGTVLEVLICFFTLWSVVGLTGFHTFLVALNQTTNEDIKGSWTGKNRVQNPYSHGNIVKNCCEVLCGPLPPSVLDRRGILPLEESGSRPPSTQETSSSLLPQSPAPTEHLNSNEMPEDSSTPEEMPPPEPPEPPQEAAEAEK

SEQ ID NO: 15 - DNA, 2789 bp, Homo sapiens
gcagtattgagttttacttcctcctctttttagtggaagacagaccataatcccagtgtgagtgaaattgattgtttcatttattaccgttttggctgggggttagttccgacaccttcacagttgaagagcaggcagaaggagttgtgaagacaggacaatcttcttggggatgctggtcctggaagccagcgggccttgctctgtctttggcctcattgaccccaggttctctggttaaaactgaaagcctactactggcctggtgcccatcaatccattgatccttgaggctgtgcccctggggcacccacctggcagggcctaccaccatgcgactgagctccctgttggctctgctgcggccagcgcttcccctcatcttagggctgtctctggggtgcagcctgagcctcctgcgggtttcctggatccagggggagggagaagatccctgtgtcgaggctgtaggggagcgaggagggccacagaatccagattcgagagctcggctagaccaaagtgatgaagacttcaaaccccggattgtcccctactacagggaccccaacaagccctacaagaaggtgctcaggactcggtacatccagacagagctgggctcccgtgagcggttgctggtggctgtcctgacctcccgagctacactgtccactttggccgtggctgtgaaccgtacggtggcccatcacttccctcggttactctacttcactgggcagcggggggcccgggctccagcagggatgcaggtggtgtctcatggggatgagcggcccgcctggctcatgtcagagaccctgcgccaccttcacacacactttggggccgactacgactggttcttcatcatgcaggatgacacatatgtgcaggccccccgcctggcagcccttgctggccacctcagcatcaaccaagacctgtacttaggccgggcagaggagttcattggcgcaggcgagcaggcccggtactgtcatgggggctttggctacctgttgtcacggagtctcctgcttcgtctgcggccacatctggatggctgccgaggagacattctcagtgcccgtcctgacgagtggcttggacgctgcctcattgactctctgggcgtcggctgtgtctcacagcaccaggggcagcagtatcgctcatttgaactggccaaaaatagggaccctgagaaggaagggagctcggctttcctgagtgccttcgccgtgcaccctgtctccgaaggtaccctcatgtaccggctccacaaacgcttcagcgctctggagttggagcgggcttacagtgaaatagaacaactgcaggctcagatccggaacctgaccgtgctgacccccgaaggggaggcagggctgagctggcccgttgggctccctgctcctttcacaccacactctcgctttgaggtgctgggctgggactacttcacagagcagcacaccttctcctgtgcagatggggctcccaagtgcccactacagggggctagcagggcggacgtgggtgatgcgttggagactgccctggagcagctcaatcggcgctatcagccccgcctgcgcttccagaagcagcgactgctcaacggctatcggcgcttcgacccagcacggggcatggagtacaccctggacctgctgttggaatgtgtgacacagcgtgggcaccggcgggccctggctcgcagggtcagcctgctgcggccactgagccgggtggaaatcctacctatgccctatgtcactgaggccacccgagtgcagctggtgctgccactcctggtggctgaagctgctgcagccccggctttcctcgaggcgtttgcagccaatgtcctggagccacgagaacatgcattgctcaccctgttgctggtctacgggccacgagaaggtggccgtggagctccagacccatttcttggggtgaaggctgcagcagcggagttagagcgacggtaccctgggacgaggctggcctggctcgctgtgcgagcagaggccccttcccaggtgcgactcatggacgtggtctcgaagaagcaccctgtggacactctcttcttccttaccaccgtgtggacaaggcctgggcccgaagtcctcaaccgctgtcgcatgaatgccatctctggctggcaggccttctttccagtccatttccaggagttcaatcctgccctgtcaccacagagatcacccccagggcccccgggggctggccctgaccccccctcccctcctggtgctgacccctcccggggggctcctataggggggagatttgaccggcaggcttctgcggagggctgcttctacaacgctgactacctggcggcccgagcccggctggcaggtgaactggcaggccaggaagaggaggaagccctggaggggctggaggtgatggatgttttcctccggttctcagggctccacctctttcgggccgtagagccagggctggtgcagaagttctccctgcgagactgcagcccacggctcagtgaagaactctaccaccgctgccgcctcagcaacctggaggggctagggggccgtgcccagctggctatggctctctttgagcaggagcaggccaatagcacttagcccgcctgggggccctaacctcattacctttcctttgtctgcctcagccccaggaagggcaaggcaagatggtggacagatagagaattgttgctgtattttttaaatatgaaaatgttattaaacatgtcttctgcc

SEQ ID NO: 16 - protein, 772 aa, Homo sapiens
MRLSSLLALLRPALPLILGLSLGCSLSLLRVSWIQGEGEDPCVEAVGERGGPQNPDSRARLDQSDEDFKPRIVPYYRDPNKPYKKVLRTRYIQTELGSRERLLVAVLTSRATLSTLAVAVNRTVAHHFPRLLYFTGQRGARAPAGMQVVSHGDERPAWLMSETLRHLHTHFGADYDWFFIMQDDTYVQAPRLAALAGHLSINQDLYLGRAEEFIGAGEQARYCHGGFGYLLSRSLLLRLRPHLDGCRGDILSARPDEWLGRCLIDSLGVGCVSQHQGQQYRSFELAKNRDPEKEGSSAFLSAFAVHPVSEGTLMYRLHKRFSALELERAYSEIEQLQAQIRNLTVLTPEGEAGLSWPVGLPAPFTPHSRFEVLGWDYFTEQHTFSCADGAPKCPLQGASRADVGDALETALEQLNRRYQPRLRFQKQRLLNGYRRFDPARGMEYTLDLLLECVTQRGHRRALARRVSLLRPLSRVEILPMPYVTEATRVQLVLPLLVAEAAAAPAFLEAFAANVLEPREHALLTLLLVYGPREGGRGAPDPFLGVKAAAAELERRYPGTRLAWLAVRAEAPSQVRLMDVVSKKHPVDTLFFLTTVWTRPGPEVLNRCRMNAISGWQAFFPVHFQEFNPALSPQRSPPGPPGAGPDPPSPPGADPSRGAPIGGRFDRQASAEGCFYNADYLAARARLAGELAGQEEEEALEGLEVMDVFLRFSGLHLFRAVEPGLVQKFSLRDCSPRLSEELYHRCRLSNLEGLGGRAQLAMALFEQEQANST

SEQ ID NO: 17 - DNA, 2142 bp, Homo sapiens
cggacgcgtgggcggacgcgtgggcggacgcgtggggccggcttggctagcgcgcggcggccgtggctaaggctgctacgaagcgagcttgggaggagcagcggcctgcggggcagaggagcatcccgtctaccaggtcccaagcggcgtggcccgcgggtcatggccaaaggagaaggcgccgagagcggctccgcggcggggctgctacccaccagcatcctccaaagcactgaacgcccggcccaggtgaagaaagaaccgaaaaagaagaaacaacagttgtctgtttgcaacaagctttgctatgcacttgggggagccccctaccaggtgacgggctgtgccctgggtttcttccttcagatctacctattggatgtggctcaggtgggccctttctctgcctccatcatcctgtttgtgggccgagcctgggatgccatcacagaccccctggtgggcctctgcatcagcaaatccccctggacctgcctgggtcgccttatgccctggatcatcttctccacgcccctggccgtcattgcctacttcctcatctggttcgtgcccgacttcccacacggccagacctattggtacctgcttttctattgcctctttgaaacaatggtcacgtgtttccatgttccctactcggctctcaccatgttcatcagcaaccgagcagactgagcgggattctgccaccgcctatcggatgactgtggaagtgctgggcacagtgctgggcacggcgatccagggacaaatcgtgggccaagcagacacgccttgtttccaggacttcaatagctctacagtagcttcacaaagtgccaaccatacacatggcaccacttcacacagggaaacgcaaaaggcatacctgctggcagcgggggtcattgtctgtatctatataatctgtgctgtcatcctgatcctgggcgtgcgggagcagagagaaccctatgaagcccagcagtctgagccaatcgcctacttccggggcctacggctggtcatgagccacggcccatacatcaaacttattactggcttcctcttcacctccttggctttcatgctggtggaggggaactttgtcttgttttgcacctacaccttgggcttccgcaatgaattccagaatctactcctggccatcatgctctcggccactttaaccattcccatctggcagtggttcttgacccggtttggcaagaagacagctgtatatgttgggatctcatcagcagtgccatttctcatcttggtggccctcatggagagtaacctcatcattacatatgcggtagctgtggcagctggcatcagtgtggcagctgccttcttactaccctggtccatgctgcctgatgtcattgacgacttccatctgaagcagccccacttccatggaaccgagcccatcttcttctccttctatgtcttcttcaccaagtttgcctctggagtgtcactgggcatttctaccctcagtctggactttgcagggtaccagacccgtggctgctcgcagccggaacgtgtcaagtttacactgaacatgctcgtgaccatggctcccatagttctcatcctgctgggcctgctgctcttcaaaatgtaccccattgatgaggagaggcggcggcagaataagaaggccctgcaggcactgagggacgaggccagcagctctggctgctcagaaacagactccacagagctggctagcatcctctagggcccgccacgttgcccgaagccaccatgcagaaggccacagaagggatcaggacctgtctgccggcttgctgagcagctggactgcaggtgctaggaagggaactgaagactcaaggaggtggcccaggacacttgctgtgctcactgtggggccggctgctctgtggcctcctgcctcccctctgcctgcctgtggggccaagccctggggctgccactgtgaatatgccaaggactgatcgggcctagcccggaacactaatgtagaaaccttttttttacagagcctaattaataacttaatgactgtgtacatagcaatgtgtgtgtatgtatatgtctgtgagctattaatgttattaattttcataaaagctggaaagc

SEQ ID NO: 18 - protein, 458 aa, Homo sapiens [sequence omitted]

SEQ ID NO: 19 - DNA, 1876 bp, Homo sapiens
ctcttttgtccaccagcccagcctgactcctggagattgtgaatagctccatccagcctgagaaacaagccgggtggctgagccaggctgtgcacggagcacctgacgggcccaacagacccatgctgcatccagagacctcccctggccgggggcatctcctggctgtgctcctggccctccttggcaccacctgggcagaggtgtggccaccccagctgcaggagcaggctccgatggccggagccctgaacaggaaggagagtttcttgctcctctccctgcacaaccgcctgcgcagctgggtccagccccctgcggctgacatgcggaggctggactggagtgacagcctggcccaactggctcaagccagggcagccctctgtggaatcccaaccccgagcctggcatccggcctgtggcgcaccctgcaagtgggctggaacatgcagctgctgcccgcgggcttggcgtcctttgttgaagtggtcagcctatggtttgcagaggggcagcggtacagccacgcggcaggagagtgtgctcgcaacgccacctgcacccactacacgcagctcgtgtgggccacctcaagccagctgggctgtgggcggcacctgtgctctgcaggccagacagcgatagaagcctttgtctgtgcctactcccccggaggcaactgggaggtcaacgggaagacaatcatcccctataagaagggtgcctggtgttcgctctgcacagccagtgtctcaggctgcttcaaagcctgggaccatgcaggggggctctgtgaggtccccaggaatccttgtcgcatgagctgccagaaccatggacgtctcaacatcagcacctgccactgccactgtccccctggctacacgggcagatactgccaagtgaggtgcagcctgcagtgtgtgcacggccggttccgggaggaggagtgctcgtgcgtctgtgacatcggctacgggggagcccagtgtgccaccaaggtgcattttcccttccacacctgtgacctgaggatcgacggagactgcttcatggtgtcttcagaggcagacacctattacagagccaggatgaaatgtcagaggaaaggcggggtgctggcccagatcaagagccagaaagtgcaggacatcctcgccttctatctgggccgcctggagaccaccaacgaggtgactgacagtgacttcgagaccaggaacttctggatcgggctcacctacaagaccgccaaggactccttccgctgggccacaggggagcaccaggccttcaccagttttgcctttgggcagcctgacaaccacgggctggtgtggctgagtgctgccatggggtttggcaactgcgtggagctgcaggcttcagctgccttcaactggaacgaccagcgctgcaaaacccgaaaccgttacatctgccagtttgcccaggagcacatctcccggtggggcccagggtcctgaggcctgaccacatggctccctcgcctgccctgggagcaccggctctgcttacctgtctgcccacctgtctggaacaagggccaggttaagaccacatgcctcatgtccaaagaggtctcagaccttgcacaatgccagaagttgggcagagagaggcagggaggccagtgagggccagggagtgagtgttagaagaagctggggcccttcgcctgcttttgattgggaagatgggcttcaattagatggcgaaggagaggacaccgccagtggtccaaaaaggctgctctcttccacctggcccagaccctgtggggcagcggagcttccctgtggcatgaaccccacggggtattaaattatgaatcagctgaaaaaaaaaaaaa

SEQ ID NO: 20 - protein, 455 aa, Homo sapiens
MLHPETSPGRGHLLAVLLALLGTTWAEVWPPQLQEQAPMAGALNRKESFLLLSLHNRLRSWVQPPAADMRRLDWSDSLAQLAQARAALCGIPTPSLASGLWRTLQVGWNMQLLPAGLASFVEVVSLWFAEGQRYSHAAGECARNATCTHYTQLVWATSSQLGCGRHLCSAGQTAIEAFVCAYSPGGNWEVNGKTIIPYKKGAWCSLCTASVSGCFKAWDHAGGLCEVPRNPCRMSCQNHGRLNISTCHCHCPPGYTGRYCQVRCSLQCVHGRFREEECSCVCDIGYGGAQCATKVHFPFHTCDLRIDGDCFMVSSEADTYYRARMKCQRKGGVLAQIKSQKVQDILAFYLGRLETTNEVTDSDFETRNFWIGLTYKTAKDSFRWATGEHQAFTSFAFGQPDNHGLVWLSAAMGFGNCVELQASAAFNWNDQRCKTRNRYICQFAQEHISRWGPGS

SEQ ID NO: 21 - DNA, 2738 bp, Homo sapiens
ggaagtccacggggagcttggatgccaaagggaggacggctgggtcctctggagaggactactcactggcatatttctgaggtatctgtagaataaccacagcctcagatactggggactttacagtcccacagaaccgtcctcccaggaagctgaatccagcaagaacaatggaggccagcgggaagctcatttgcagacaaaggcaagtccttttttcctttctccttttgggcttatctctggcgggcgcggcggaacctagaagctattctgtggtggaggaaactgagggcagctcctttgtcaccaatttagcaaaggacctgggtctggagcagagggaattctccaggcggggggttagggttgtttccagagggaacaaactacatttgcagctcaatcaggagaccgcggatttgttgctaaatgagaaattggaccgtgaggatctgtgcggtcacacagagccctgtgtgctacgtttccaagtgttgctagagagtcccttcgagttttttcaagctgagctgcaagtaatagacataaacgaccactctccagtatttctggacaaacaaatgttggtgaaagtatcagagagcagtcctcctgggactacgtttcctctgaagaatgccgaagacttagatgtaggccaaaacaatattgagaactatataatcagccccaactcctattttcgggtcctcacccgcaaacgcagtgatggcaggaaatacccagagctggtgctggacaaagcgctggaccgagaggaagaagctgagctcaggttaacactcacagcactggatggtggctctccgcccagatctggcactgctcaggtctacatcgaagtcctggatgtcaacgataatgcccctgaatttgagcagcctttctatagagtgcagatctctgaggacagtccggtaggcttcctggttgtgaaggtctctgccacggatgtagacacaggagtcaacggagagatttcctattcacttttccaagcttcagaagagattggcaaaacctttaagatcaatcccttgacaggagaaattgaactaaaaaaacaactcgatttcgaaaaacttcagtcctatgaagtcaatattgaggcaagagatgctggaaccttttctggaaaatgcaccgttctgattcaagtgatagatgtgaacgaccatgccccagaagttaccatgtctgcatttaccagcccaatacctgagaacgcgcctgaaactgtggttgcacttttcagtgtttcagatcttgattcaggagaaaatgggaaaattagttgctccattcaggaggatctacccttcctcctgaaatccgcggaaaacttttacaccctactaacggagagaccactagacagagaaagcagagcggaatacaacatcactatcactgtcactgacttggggacccctatgctgataacacagctcaatatgaccgtgctgatcgccgatgtcaatgacaacgctcccgccttcacccaaacctcctacaccctgttcgtccgcgagaacaacagccccgccctgcacatccgcagcgtcagcgctacagacagagactcaggcaccaacgcccaggtcacctactcgctgctgccgccccaggacccgcacctgcccctcacatccctggtctccatcaacgcggacaacggccacctgttcgccctcaggtctctggactacgaggccctgcaggggttccagttccgcgtgggcgcttcagaccacggctccccggcgctgagcagcgaggcgctggtgcgcgtggtggtgctggacgccaacgacaactcgcccttcgtgctgtacccgctgcagaacggctccgcgccctgcaccgagctggtgccccgggcggccgagccgggctacctggtgaccaaggtggtggcggtggacggcgactcgggccagaacgcctggctgtcgtaccagctgctcaaggccacggagctcggtctgttcggcgtgtgggcgcacaatggcgaggtgcgcaccgccaggctgctgagcgagcgcgacgcggccaagcacaggctggtggtgctggtcaaggacaatggcgagcctccgcgctcggccaccgccacgctgcacgtgctcctggtggacggcttctcccagccctacctgcctctcccggaggcggccccgacccaggcccaggccgacttgctcaccgtctacctggtggtggcgttggcctcggtgtcttcgctcttcctcttttcggtgctcctgttcgtggcggtgcggctgtgtaggaggagcagggcggcctcggtgggtcgctgcttggtgcccgagggcccccttccagggcatcttgtggacatgagcggcaccaggaccctatcccagagctaccagtatgaggtgtgtctggcaggaggctcagggaccaatgagttcaagttcctgaagccgattatccccaacttccctccccagtgccctgggaaagaaatacaaggaaattctaccttccccaataactttgggttcaatattcagtgaccatagttgacttttacattccataggtattttattttgtggcatttccatgccaatgtttatttcccccaatttgtgtgtatgtaatattgtacggatttactcttgatttttctcatgttctttctccctttgttttaaagtgaacatttacctttattcctggttctt

SEQ ID NO: 22 - protein, 798 aa, Homo sapiens
MEASGKLICRQRQVLFSFLLLGLSLAGAAEPRSYSVVEETEGSSFVTNLAKDLGLEQREFSRRGVRVVSRGNKLHLQLNQETADLLLNEKLDREDLCGHTEPCVLRFQVLLESPFEFFQAELQVIDINDHSPVFLDKQMLVKVSESSPPGTTFPLKNAEDLDVGQNNIENYIISPNSYFRVLTRKRSDGRKYPELVLDKALDREEEAELRLTLTALDGGSPPRSGTAQVYIEVLDVNDNAPEFEQPFYRVQISEDSPVGFLVVKVSATDVDTGVNGEISYSLFQASEEIGKTFKINPLTGEIELKKQLDFEKLQSYEVNIEARDAGTFSGKCTVLIQVIDVNDHAPEVTMSAFTSPIPENAPETVVALFSVSDLDSGENGKISCSIQEDLPFLLKSAENFYTLLTERPLDRESRAEYNITITVTDLGTPMLITQLNMTVLIADVNDNAPAFTQTSYTLFVRENNSPALHIRSVSATDRDSGTNAQVTYSLLPPQDPHLPLTSLVSINADNGHLFALRSLDYEALQGFQFRVGASDHGSPALSSEALVRVVVLDANDNSPFVLYPLQNGSAPCTELVPRAAEPGYLVTKVVAVDGDSGQNAWLSYQLLKATELGLFGVWAHNGEVRTARLLSERDAAKHRLVVLVKDNGEPPRSATATLHVLLVDGFSQPYLPLPEAAPTQAQADLLTVYLVVALASVSSLFLFSVLLFVAVRLCRRSRAASVGRCLVPEGPLPGHLVDMSGTRTLSQSYQYEVCLAGGSGTNEFKFLKPIIPNFPPQCPGKEIQGNSTFPNNFGFNIQ

SEQ ID NO: 23 - DNA, 971 bp, Homo sapiens
aacaaagttcagtgactgagagggctgagcggaggctgctgaaggggagaaaggagtgaggagctgctgggcagagagggactgtccggctcccagatgctgggcctcctggggagcacagccctcgtgggatggatcacaggtgctgctgtggcggtcctgctgctgctgctgctgctggccacctgccttttccacggacggcaggactgtgacgtggagaggaaccgtacagctgcagggggaaaccgagtccgccgggcccagccttggcccttccggcggcggggccacctgggaatctttcaccatcaccgtcatcctggccacgtatctcatgtgccgaatgtgggcctccaccaccaccaccacccccgccacacccctcaccacctccaccaccaccaccacccccaccgccaccatccccgccacgctcgctgaggctgctgtcgccggtgcctgtggacagcagctgcccctgccctcccatctgttcccaggacaagtggaccccatgtttccatgtggaaggatgcatctctggggtgaacgaggggaacaatagactggggcttgctccagctgcatttgcatggcatgccccagtgtactatggcagcagagaatggaggaacactgggtctgcagtgctgaagggtttggggagtggagagcaagggtgctctttcggggctggacagcccgtcttgtgacagtgactcccagtgagccccagaaatgacaagcgtgtcttggcagagccagcacacaagtggatgtgaagtgcccgtcttgacctcctcatcaggctgctgcaggcctctggcgggcagggcactgggagaggccctgagaatgtccttttggtttggagaaggcagtgtgaggctgcacagtcaattcatcggtgccttagtccaagaaaataaaaaccactaagaagctttaaaaaaaaaaaaaaaaaaaaa

SEQ ID NO: 24 - protein, 115 aa, Homo sapiens
MLGLLGSTALVGWITGAAVAVLLLLLLLATCLFHGRQDCDVERNRTAAGGNRVRRAQPWPFRRRGHLGIFHHHRHPGHVSHVPNVGLHHHHHPRHTPHHLHHHHHPHRHHPRHAR

SEQ ID NO: 25 - DNA, 1316 bp, Homo sapiens
ctgctgcatccgggtgtctggaggctgtggccgttttgttttcttggctaaaatcgggggagtgaggcgggccggcgcggcgcgacaccgggctccggaaccactgcacgacggggctggactgacctgaaaaaaatgtctggatttctagagggcttgagatgctcagaatgcattgactggggggaaaagcgcaatactattgcttccattgctgctggtgtactattttttacaggctggtggattatcatagatgcagctgttatttatcccaccatgaaagatttcaaccactcataccatgcctgtggtgttatagcaaccatagccttcctaatgattaatgcagtatcgaatggacaagtccgaggtgatagttacagtgaaggttgtctgggtcaaacaggtgctcgcatttggcttttcgttggtttcatgttggcctttggatctctgattgcatctatgtggattctttttggaggttatgttgctaaagaaaaagacatagtataccctggaattgctgtatttttccagaatgccttcatcttttttggagggctggtttttaagtttggccgcactgaagacttatggcagtgaacacatctgatttcccacagcacaacagccctgcatgggtttgtttgtttttttactgctcactcccaaccttttgtaatgccattttctaaacttatttctgagtgtagtctcagcttaaagttgtgtaatactaaaatcacgagaacacctaaacaacaaccaaaaatctattgtggtatgcacttgattaacttataaaatgttagaggaaactttcacatgaataatttttgtcaaattttatcatggtataatttgtaaaaataaaaagaaattacaaaagaaattatggatttgtcaatgtaagtatttgtcatatctgaggtccaaaaccacaatgaaagtgctctgaagatttaatgtgtttattcaaatgtggtctcttctgtgtcaaatgttaaatgaaatataaacattttttagtttttaaaatattccgtggtcaaaattcttcctcactataattggtatttacttttaccaaaaattctgtgaacatgtaatgtaactggcttttgagggtctcccaaggggtgagtggacgtgttggaagagagaagcaccatggtccagccaccaggctccctgtgtcccttccatgggaaggtcttccgctgtgcctctcattccaagggcaggaagatgtgactcagccatgacacgtggttctggtgggatgcacagtcactccacatccaccactg

SEQ ID NO: 26 - protein, 157 aa, Homo sapiens
MSGFLEGLRCSECIDWGEKRNTIASIAAGVLFFTGWWIIIDAAVIYPTMKDFNHSYHACGVIATIAFLMINAVSNGQVRGDSYSEGCLGQTGARIWLFVGFMLAFGSLIASMWILFGGYVAKEKDIVYPGIAVFFQNAFIFFGGLVFKFGRTEDLWQ

SEQ ID NO: 27 - DNA, 1371 bp, Homo sapiens
cagagcagataatggcaagcatggctgccgtgctcacctgggctctggctcttctttcagcgttttcggccacccaggcacggaaaggcttctgggactacttcagccagaccagcggggacaaaggcagggtggagcagatccatcagcagaagatggctcgcgagcccgcgaccctgaaagacagccttgagcaagacctcaacaatatgaacaagttcctggaaaagctgaggcctctgagtgggagcgaggctcctcggctcccacaggacccggtgggcatgcggcggcagctgcaggaggagttggaggaggtgaaggctcgcctccagccctacatggcagaggcgcacgagctggtgggctggaatttggagggcttgcggcagcaactgaagccctacacgatggatctgatggagcaggtggccctgcgcgtgcaggagctgcaggagcagttgcgcgtggtgggggaagacaccaaggcccagttgctggggggcgtggacgaggcttgggctttgctgcagggactgcagagccgcgtggtgcaccacaccggccgcttcaaagagctcttccacccatacgccgagagcctggtgagcggcatcgggcgccacgtgcaggagctgcaccgcagtgtggctccgcacgcccccgccagccccgcgcgcctcagtcgctgcgtgcaggtgctctcccggaagctcacgctcaaggccaaggccctgcacgcacgcatccagcagaacctggaccagctgcgcgaagagctcagcagagcctttgcaggcactgggactgaggaaggggccggcccggacccctagatgctctccgaggaggtgcgccagcgacttcaggctttccgccaggacacctacctgcagatagctgccttcactcgcgccatcgaccaggagactgaggaggtccagcagcagctggcgccacctccaccaggccacagtgccttcgccccagagtttcaacaaacagacagtggcaaggttctgagcaagctgcaggcccgtctggatgacctgtgggaagacatcactcacagccttcatgaccagggccacagccatctgggggacccctgaggatctacctgcccaggcccattcccagcttcttgtctggggagccttggctctgagcctctagcatggttcagtccttgaaagtggcctgttgggtggagggtggaaggtcctgtgcaggacagggaggccaccaaaggggctgctgtctcctgcatatccagcctcctgcgactccccaatctggatgcattacattcaccaggctttgcaaaaaaaaaaaaaaaaaaaaaaaaaaaaaaaaaaaaaaaaaaaaaaa

SEQ ID NO: 28 - protein, 274 aa, Homo sapiens
MASMAAVLTWALALLSAFSATQARKGFWDYFSQTSGDKGRVEQIHQQKMAREPATLKDSLEQDLNNMNKFLEKLRPLSGSEAPRLPQDPVGMRRQLQEELEEVKARLQPYMAEAHELVGWNLEGLRQQLKPYTMDLMEQVALRVQELQEQLRVVGEDTKAQLLGGVDEAWALLQGLQSRVVHHTGRFKELFHPYAESLVSGIGRHVQELHRSVAPHAPASPARLSRCVQVLSRKLTLKAKALHARIQQNLDQLREELSRAFAGTGTEEGAGPDP

SEQ ID NO: 29 - DNA, 3741 bp, Homo sapiens
cgggacaaggacatagggctgagagtagccatgggctctggaggagacagcctcctggggggcaggggttccctgcctctgctgctcctgctcatcatgggaggcatggctcaggactccccgccccagatcttagtccacccccaggaccagctgttccagggccctggccctgccaggatgagctgccgagcctcaggccagccacctcccaccatccgctggttgctgaatgggcagcccctgagcatggtgcccccagacccacaccacctcctgcctgatgggacccttctgctgctacagccccctgcccggggacatgcccacgatggccaggccctgtccacagacctgggtgtctacacatgtgaggccagcaaccggcttggcacggcagtcagcagaggcgctcggctgtctgtggctgtcctccgggaggatttccagatccagcctcgggacatggtggctgtggtgggtgagcagtttactctggaatgtgggccgccctggggccacccagagcccacagtctcatggtggaaagatgggaaacccctggccctccagcccggaaggcacacagtgtccggggggtccctgctgatggcaagagcagagaagagtgacgaagggacctacatgtgtgtggccaccaacagcgcaggacacagggagagccgcgcagcccgggtttccatccaggagccccaggactacacggagcctgtggagcttctggctgtgcgaattcagctggaaaatgtgacactgctgaacccggatcctgcagagggccccaagcctagaccggcggtgtggctcagctggaaggtcagtggccctgctgcgcctgcccaatcttacacggccttgttcaggacccagactgccccgggaggccagggagctccgtgggcagaggagctgctggccggctggcagagcgcagagcttggaggcctccactggggccaagactacgagttcaaagtgagaccatcctctggccgggctcgaggccctgacagcaacgtgctgctcctgaggctgccggaaaaagtgcccagtgccccacctcaggaagtgactctaaagcctggcaatggcactgtctttgtgagctgggtcccaccacctgctgaaaaccacaatggcatcatccgtggctaccaggtctggagcctgggcaacacatcactgccaccagccaactggactgtagttggtgagcagacccagctggaaatcgccacccatatgccaggctcctactgcgtgcaagtggctgcagtcactggtgctggagctggggagcccagtagacctgtctgcctccttttagagcaggccatggagcgagccacccaagaacccagtgagcatggtccctggaccctggagcagctgagggctaccttgaagcggcctgaggtcattgccacctgcggtgttgcactctggctgctgcttctgggcaccgccgtgtgtatccaccgccggcgccgagctagggtgcacctgggcccaggtctgtacagatataccagtgaggatgccatcctaaaacacaggatggatcacagtgactcccagtggttggcagacacttggcgttccacctctggctctcgggacctgagcagcagcagcagcctcagcagtcggctgggggcggatgcccgggacccactagactgtcgtcgctccttgctctcctgggactcccgaagccccggcgtgcccctgcttccagacaccagcactttttatggctccctcatcgctgagctgccctccagtaccccagccaggccaagtccccaggtcccagctgtcaggcgcctcccaccccagctggcccagctctccagcccctgttccagctcagacagcctctgcagccgcaggggactctcttctccccgcttgtctctggcccctgcagaggcttggaaggccaaaaagaagcaggagctgcagcatgccaacagttccccactgctccggggcagccactccttggagctccgggcctgtgagttaggaaatagaggttccaagaacctttcccaaagcccaggagctgtgccccaagctctggttgcctggcgggccctgggaccgaaactcctcagctcctcaaatgagctggttactcgtcatctccctccagcacccctctttcctcatgaaactcccccaactcagagtcaacagacccagcctccggtggcaccacaggctccctcctccatcctgctgccagcagcccccatccccatccttagcccctgcagtccccctagcccccaggcctcttccctctctggccccagcccagcttccagtcgcctgtccagctcctcactgtcatccctgggggaggatcaagacagcgtgctgacccctgaggaggtagccctgtgcttggaactcagtgagggtgaggagactcccaggaacagcgtctctcccatgccaagggctccttcaccccccaccacctatgggtacatcagcgtcccaacagcctcagagttcacggacatgggcaggactggaggaggggtggggcccaaggggggagtcttgctgtgcccacctcggccctgcctcacccccacccccagcgagggctccttagccaatggttggggctcagcctctgaggacaatgccgccagcgccagagccagccttgtcagctcctccgatggctccttcctcgctgatgctcactttgcccgggccctggcagtggctgtggatagctttggtttcggtctagagcccagggaggcagactgcgtcttcatagatgcctcatcacctccctccccacgggatgagatcttcctgacccccaacctctccctgcccctgtgggagtggaggccagactggttggaagacatggaggtcagccacacccagcggctgggaagggggatgcctccctggccccctgactctcagatctcttcccagagaagtcagctccactgtcgtatgcccaaggctggtgcttctcctgtagattactcctgaaccgtgtccctgagacttcccagacgggaatcagaaccacttctcctgtccacccacaagacctgggctgtggtgtgtgggtcttggcctgtgtttctctgcagctggggtccaccttcccaagcctccagagagttctccctccacgattgtgaaaacaaatgaaaacaaaattagagcaaagctgacctggagccctcagggagcaaaacatcatctccacctgactcctagccactgctttctcctctgtgccatccactcccaccaccaggttgttttggcctgaggagcagccctgcctgctgctcttcccccaccatttggatcacaggaagtggaggagccagaggtgcctttgtggaggacagcagtggctgctgggagagggctgtggaggaaggagcttctcggagccccctctcagccttacctgggcccctcctctagagaagagctcaactctctcccaacctcaccatggaaagaaaataattatgaatgccactgaggcactgaggccctacctcatgccaaacaaagggttcaaggctgggtctagcgaggatgctgaaggaagggaggtatgagaccgtaggtcaaaagcaccatcctcgtactgttgtcactatgagcttaagaaatttgataccataaaatggtaaagacttgaaaaaaaaaaaaaaa

SEQ ID NO: 30 - protein, 1007 aa, Homo sapiens [sequence omitted]

SEQ ID NO: 31 - DNA, 1894 bp, Homo sapiens
ggcggcgtccgtgaggggctcctttgggcaggggtagtgtttggtgtccctgtcttgcgtgatattgacaaactgaagctttcctgcaccactggacttaaggaagagtgtactcgtaggcggacagctttagtggccggccggccgctctcatcccccgtaaggagcagagtcctttgtactgaccaagatgagcaacatctacatccaggagcctcccacgaatgggaaggttttattgaaaactacagctggagatattgacatagagttgtggtccaaagaagctcctaaagcttgcagaaattttatccaactttgtttggaagcttattatgacaataccatttttcatagagttgtgcctggtttcatagtccaaggcggagatcctactggcacagggagtggtggagagtctatctatggagcgccattcaaagatgaatttcattcacggttgcgttttaatcggagaggactggttgccatggcaaatgctggttctcatgataatggcagccagtttttcttcacactgggtcgagcagatgaacttaacaataagcataccatctttggaaaggttacaggggatacagtatataacatgttgcgactgtcagaagtagacattgatgatgacgaaagaccacataatccacacaaaataaaaagctgtgaggttttgtttaatccttttgatgacatcattccaagggaaattaaaaggctgaaaaaagagaaaccagaggaggaagtaaagaaattgaaacccaaaggcacaaaaaattttagtttactttcatttggagaggaagctgaggaagaagaggaggaagtaaatcgagttagtcagagcatgaagggcaaaagcaaaagtagtcatgacttgcttaaggatgatccacatctcagttctgttccagttgtagaaagtgaaaaaggtgatgcaccagatttagttgatgatggagaagatgaaagtgcagagcatgatgaatatattgatggtgatgaaaagaacctgatgagagaaagaattgccaaaaaattaaaaaaggacacaagtgcgaatgttaaatcagctggagaaggagaagtggagaagaaatcagtcagccgcagtgaagagctcagaaaagaagcaagacaattaaaacgggaactcttagcagcaaaacaaaaaaaagtagaaaatgcagcaaaacaagcagaaaaaagaagtgaagaggaagaagcccctccagatggtgctgttgccgaatacagaagagaaaagcaaaagtatgaagctttgaggaagcaacagtcaaagaagggaacttcccgggaagatcagacccttgcactgctgaaccagtttaaatctaaactcactcaagcaattgctgaaacacctgaaaatgacattcctgaaacagaagtagaagatgatgaaggatggatgtcacatgtacttcagtttgaggataaaagcagaaaagtgaaagatgcaagcatgcaagactcagatacatttgaaatctatgatcctcggaatccagtgaataaaagaaggagggaagaaagcaaaaagctgatgagagagaaaaaagaaagaagataaaatgagaataatgataaccagaacttgctggaaatgtgcctacaatggccttgtaacagccattgttcccaacagcatcacttaggggtgtgaaaagaagtatttttgaacctgttgtctggttttgaaaaacaattatcttgttttgcaaattgtggaatgatgtaagcaaatgcttttggttactggtacatgtgttttttcctagctgaccttttatattgctaaatctgaaataaaataactttccttccacaaaaaaaaaaaaaaaaaaaaaaaaaaaaa

SEQ ID NO: 32 - protein, 472 aa, Homo sapiens
MSNIYIQEPPTNGKVLLKTTAGDIDIELWSKEAPKACRNFIQLCLEAYYDNTIFHRVVPGFIVQGGDPTGTGSGGESIYGAPFKDEFHSRLRFNRRGLVAMANAGSHDNGSQFFFTLGRADELNNKHTIFGKVTGDTVYNMLRLSEVDIDDDERPHNPHKIKSCEVLFNPFDDIIPREIKRLKKEKPEEEVKKLKPKGTKNFSLLSFGEEAEEEEEEVNRVSQSMKGKSKSSHDLLKDDPHLSSVPVVESEKGDAPDLVDDGEDESAEHDEYIDGDEKNLMRERIAKKLKKDTSANVKSAGEGEVEKKSVSRSEELRKEARQLKRELLAAKQKKVENAAKQAEKRSEEEEAPPDGAVAEYRREKQKYEALRKQQSKKGTSREDQTLALLNQFKSKLTQAIAETPENDIPETEVEDDEGWMSHVLQFEDKSRKVKDASMQDSDTFEIYDPRNPVNKRRREESKKLMREKKERR

SEQ ID NO: 33 - DNA, 3004 bp, Homo sapiens
cgacgccggcgtgatgtggcttccgctggtgctgctcctggctgtgctgctgctggccgtcctctgcaaagtttacttgggactattctctggcagctccccgaatcctttctccgaagatgtcaaacggcccccagcgcccctggtaactgacaaggaggccaggaagaaggttctcaaacaagctttttcagccaaccaagtgccggagaagctggatgtggtggtaattggcagtggctttgggggcctggctgcagctgcaattctagctaaagctggcaagcgagtcctggtgctggaacaacataccaaggcagggggctgctgtcatacctttggaaagaatggccttgaatttgacacaggaatccattacattgggcgtatggaagagggcagcattggccgttttatcttggaccagatcactgaagggcagctggactgggctcccctgtcctctccttttgacatcatggtactggaagggcccaatggccgaaaggagtaccccatgtacagtggagagaaagcctacattcagggcctcaaggagaagtttccacaggaggaagctatcattgacaagtatataaagctggttaaggtggtatccagtggagcccctcatgccatcctgttgaaattcctcccattgcccgtggttcagctcctcgacaggtgtgggctgctgactcgtttctctccattccttcaagcatccacccagagcctggctgaggtcctgcagcagctgggggcctcctctgagctccaggcagtactcagctacatcttccccacttacggtgtcacccccaaccacagtgccttttccatgcacgccctgctggtcaaccactacatgaaaggaggcttttatccccgagggggttccagtgaaattgccttccacaccatccctgtgattcagcgggctgggggcgctgtcctcacaaaggccactgtgcagagtgtgttgctggactcagctgggaaagcctgtggtgtcagtgtgaagaaggggcatgagctggtgaacatctattgccccatcgtggtctccaacgcaggactgttcaacacctatgaacacctactgccggggaacgcccgctgcctgccaggtgtgaagcagcaactggggacggtgcggcccggcttaggcatgacctctgttttcatctgcctgcgaggcaccaaggaagacctgcatctgccgtccaccaactactatgtttactatgacacggacatggaccaggcgatggagcgctacgtctccatgcccagggaagaggctgcggaacacatccctcttctcttcttcgctttcccatcagccaaagatccgacctgggaggaccgattcccaggccggtccaccatgatcatgctcatacccactgcctacgagtggtttgaggagtggcaggcggagctgaagggaaagcggggcagtgactatgagaccttcaaaaactcctttgtggaagcctctatgtcagtggtcctgaaactgttcccacagctggaggggaaggtggagagtgtgactgcaggatccccactcaccaaccagttctatctggctgctccccgaggtgcctgctacggggctgaccatgacctgggccgcctgcacccttgtgtgatggcctccttgagggcccagagccccatccccaacctctatctgacaggccaggatatcttcacctgtggactggtcggggccctgcaaggtgccctgctgtgcagcagcgccatcctgaagcggaacttgtactcagaccttaagaatcttgattctaggatccgggcacagaagaaaaagaattagttccatcagggaggagtcagaggaatttgcccaatggctggggcatctcccttgacttacccataatgtctttctgcattagttccttgcacgtataaagcactctaatttggttctgatgcctgaagagaggcctagtttaaatcacaattccgaatctggggcaatggaatcactgcttccagctggggcaggtgagatctttacgccttttataacatgccatccctactaataggatattgacttggatagcttgatgtctcatgacgagcggcgctctgcatccctcacccatgcctcctaactcagtgatcaaagcgaatattccatctgtggatagaacccctggcagtgttgtcagctcaacctggtgggttcagttctgtcctgaggcttctgctctcattcatttagtgctacgctgcacagttctacactgtcaagggaaaagggagactaatgaggcttaactcaaaacctgggcgtggttttggttgccattccataggtttggagagctctagatctcttttgtgctgggttcagtggctcttcaggggacaggaaatgcctgtgtctggccagtgtggttctggagctttggggtaacagcaggatccatcagttagtagggtgcatgtcagatgatcatatccaattcatatggaagtcccgggtctgtcttccttatcatcggggtggcagctggttctcaatgtgccagcagggactcagtacctgagcctcaatcaagccttatccaccaaatacacagggaagggtgatgcagggaagggtgacatcaggagtcagggcatggactggtaagatgaatactttgctgggctgaagcaggctgcagggcattccagccaagggcacagcaggggacagtgcagggaggtgtggggtaagggagggaagtcacatcagaaaagggaaagccacggaatgtgtgtgaagcccagaaatggcatttgcagttaattagcacatgtgagggttagacaggtaggtgaatgcaagctcaaggtttggaaaaatgacttttcagttatgtctttggtatcagacatacgaaaggtctctttgtagttcgtgttaatgtaacattaataaatttattgattccattgctttaaaaaaaaaaaaaaa

SEQ ID NO: 34 - protein, 610 aa, Homo sapiens
MWLPLVLLLAVLLLAVLCKVYLGLFSGSSPNPFSEDVKRPPAPLVTDKEARKKVLKQAFSANQVPEKLDVVVIGSGFGGLAAAAILAKAGKRVLVLEQHTKAGGCCHTFGKNGLEFDTGIHYIGRMEEGSIGRFILDQITEGQLDWAPLSSPFDIMVLEGPNGRKEYPMYSGEKAYIQGLKEKFPQEEAIIDKYIKLVKVVSSGAPHAILLKFLPLPVVQLLDRCGLLTRFSPFLQASTQSLAEVLQQLGASSELQAVLSYIFPTYGVTPNHSAFSMHALLVNHYMKGGFYPRGGSSEIAFHTIPVIQRAGGAVLTKATVQSVLLDSAGKACGVSVKKGHELVNIYCPIVVSNAGLFNTYEHLLPGNARCLPGVKQQLGTVRPGLGMTSVFICLRGTKEDLHLPSTNYYVYYDTDMDQAMERYVSMPREEAAEHIPLLFFAFPSAKDPTWEDRFPGRSTMIMLIPTAYEWFEEWQAELKGKRGSDYETFKNSFVEASMSVVLKLFPQLEGKVESVTAGSPLTNQFYLAAPRGACYGADHDLGRLHPCVMASLRAQSPIPNLYLTGQDIFTCGLVGALQGALLCSSAILKRNLYSDLKNLDSRIRAQKKKN

SEQ ID NO: 35 - DNA, 773 bp, Homo sapiens
ccaaagtgatcatttgaaaaagagatatccacatcttcaagcccatataaaggatagaagctgcacagggcagctttacttactccagcaccttcctctcccaggcaaatggtgctgaccatctttgggatacaatctcatggatacgaggtttttaacatcatcagcccaagcaacaatggtggcaatgttcaggagacagtgacaattgataatgaaaaaaataccgccatcgttaacatccatgcaggatcatgctcttctaccacaatttttgactataaacatggctacattgcatccagggtgctctcccgaagagcctgctttatcctgaagatggaccatcagaacatccctcctctgaacaatctccaatggtacatctatgagaaacaggctctggacaacatgttctccaacaaatacacctgggtcaagtacaaccctctggagtctctgatcaaagacgtggattggttcctgcttgggtcacccattgagaaactctgcaaacatatccctttgtataagggggaagtggttgaaaacacacataatgtcggtgctggaggctgtgcaaaggctgggctcctgggcatcttgggaatttcaatctgtgcagacattcatgtttaggatgattagccctcttgttttatcttttcaaagaaatacatccttggtttacactcaaaagtcaaattaaattctttcccaatgccccaactaattttgagattcagtcagaaaatataaatgctgtatttata

SEQ ID NO: 36 - protein, 184 aa, Homo sapiens
MKILVAFLVVLTIFGIQSHGYEVFNIISPSNNGGNVQETVTIDNEKNTAIVNIHAGSCSSTTIFDYKHGYIASRVLSRRACFILKMDHQNIPPLNNLQWYIYEKQALDNMFSNKYTWVKYNPLESLIKDVDWFLLGSPIEKLCKHIPLYKGEVVENTHNVGAGGCAKAGLLGILGISICADIHV

SEQ ID NO: 37 - DNA, 1227 bp, Homo sapiens
cggacgcgtgggccgccacctccggaacaagccatggtggcggcgacggtggcagcggcgtggctgctcctgtgggctgcggcctgcgcgcagcaggagcaggacttctacgacttcaaggcggtcaacatccggggcaaactggtgtcgctggagaagtaccgcggatcggtgtccctggtggtgaatgtggccagcgagtgcggcttcacagaccagcactaccgagccctgcagcagctgcagcgagacctgggcccccaccactttaacgtgctcgccttcccctgcaaccagtttggccaacaggagcctgacagcaacaaggagattgagagctttgcccgccgcacctacagtgtctcattccccatgtttagcaagattgcagtcaccggtactggtgcccatcctgccttcaagtacctggcccagacttctgggaaggagcccacctggaacttctggaagtacctagtagccccagatggaaaggtggtaggggcttgggacccaactgtgtcagtggaggaggtcagaccccagatcacagcgctcgtgaggaagctcatcctactgaagcgagaagacttataaccaccgcgtctcctcctccaccacctcatcccgcccacctgtgtggggctgaccaatgcaaactcaaatggtgcttcaaagggagagacccactgactctccttcctttactcttatgccattggtcccatcattcttgtgggggaaaaattctagtattttgattatttgaatcttacagcaacaaataggaactcctggccaatgagagctcttgaccagtgaatcaccagccgatacgaacgtcttgccaacaaaaatgtgtggcaaatagaagtatatcaagcaataatctcccacccaaggcttctgtaaactgggaccaatgattacctcatagggctgttgtgaggattaggatgaaatacctgtgaaagtgcctaggcagtgccagccaaataggaggcattcaatgaacattttttgcatataaaccaaaaaataacttgttatcaataaaaacttgcatccaacatgaatttccagccgatgataatccaggccaaaggtttagttgttgttatttcctctgtattattttcttcattacaaaagaaatgcaagttcattgtaacaatccaaacaatacctcacgatataaaataaaaatgaaagtatcctcctcaaaaa

SEQ ID NO: 38 - protein, 187 aa, Homo sapiens
MVAATVAAAWLLLWAAACAQQEQDFYDFKAVNIRGKLVSLEKYRGSVSLVVNVASECGFTDQHYRALQQLQRDLGPHHFNVLAFPCNQFGQQEPDSNKEIESFARRTYSVSFPMFSKIAVTGTGAHPAFKYLAQTSGKEPTWNFWKYLVAPDGKVVGAWDPTVSVEEVRPQITALVRKLILLKREDL

SEQ ID NO: 39 - DNA, 2692 bp, Homo sapiens
cccgggtcgacccacgcgtccggggagaaaggatggccggcctggcggcgcggttggtcctgctagctggggcagcggcgctggcgagcggctcccagggcgaccgtgagccggtgtaccgcgactgcgtactgcagtgcgaagagcagaactgctctgggggcgctctgaatcacttccgctcccgccagccaatctacatgagtctagcaggctggacctgtcgggacgactgtaagtatgagtgtatgtgggtcaccgttgggctctacctccaggaaggtcacaaagtgcctcagttccatggcaagtggcccttctcccggttcctgttctttcaagagccggcatcggccgtggcctcgtttctcaatggcctggccagcctggtgatgctctgccgctaccgcaccttcgtgccagcctcctcccccatgtaccacacctgtgtggccttcgcctgggtgtccctcaatgcatggttctggtccacagtcttccacaccagggacactgacctcacagagaaaatggactacttctgtgcctccactgtcatcctacactcaatctacctgtgctgcgtcaggaccgtggggctgcagcacccagctgtggtcagtgccttccgggctctcctgctgctcatgctgaccgtgcacgtctcctacctgagcctcatccgcttcgactatggctacaacctggtggccaacgtggctattggcctggtcaacgtggtgtggtggctggcctggtgcctgtggaaccagcggcggctgcctcacgtgcgcaagtgcgtggtggtggtcttgctgctgcaggggctgtccctgctcgagctgcttgacttcccaccgctcttctgggtcctggatgcccatgccatctggcacatcagcaccatccctgtccacgtcctctttttcagctttctggaagatgacagcctgtacctgctgaaggaatcagaggacaagttcaagctggactgaagaccttggagcgagtctgccccagtggggatcctgcccccgccctgctggcctcccttctcccctcaacccttgagatgattttctcttttcaacttcttgaacttggacatgaaggatgtgggcccagaatcatgtggccagcccaccccctgttggccctcaccagccttggagtctgttctagggaaggcctcccagcatctgggactcgagagtgggcagcccctctacctcctggagctgaactggggtggaactgagtgtgttcttagctctaccgggaggacagctgcctgtttcctccccaccagcctcctccccacatccccagctgcctggctgggtcctgaagccctctgtctacctgggagaccagggaccacaggccttagggatacagggggtccccttctgttaccaccccccaccctcctccaggacaccactaggtggtgctggatgcttgttctttggccagccaaggttcacggcgattctccccatgggatcttgagggaccaagctgctgggattgggaaggagtttcaccctgaccgttgccctagccaggttcccaggaggcctcaccatactccctttcagggccagggctccagcaagcccagggcaaggatcctgtgctgctgtctggttgagagcctgccaccgtgtgtcgggagtgtgggccaggctgagtgcataggtgacagggccgtgagcatgggcctgggtgtgtgtgagctcaggcctaggtgcgcagtgtggagacgggtgttgtcggggaagaggtgtggcttcaaagtgtgtgtgtgcagggggtgggtgtgttagcgtgggttaggggaacgtgtgtgcgcgtgctggtgggcatgtgagatgagtgactgccggtgaatgtgtccacagttgagaggttggagcaggatgagggaatcctgtcaccatcaataatcacttgtggagcgccagctctgcccaagacgccacctgggcggacagccaggagctctccatggccaggctgcctgtgtgcatgttccctgtctggtgcccctttgcccgcctcctgcaaacctcacagggtccccacacaacagtgccctccagaagcagcccctcggaggcagaggaaggaaaatggggatggctggggctctctccatcctccttttctccttgccttcgcatggctggccttcccctccaaaacctccattcccctgctgccagcccctttgccatagcctgattttggggaggaggaaggggcgatttgagggagaaggggagaaagcttatggctgggtctggtttcttcccttcccagagggtcttactgttccagggtggccccagggcaggcaggggccacactatgcctgtgccctggtaaaggtgacccctgccatttaccagcagccctggcatgttcctgccccacaggaatagaatggagggagctccagaaactttccatcccaaaggcagtctccgtggttgaagcagactggatttttgctctgcccctgaccccttgtccctctttgagggaggggagctatgctaggactccaacctcagggactcgggtggcctgcgctagcttcttttgatactgaaaacttttaaggtgggagggtggcaagggatgtgcttaataaatcaattccaagcctcaaaaaaaaaaaaaaaaa

SEQ ID NO: 40 - protein, 320 aa, Homo sapiens
MAGLAARLVLLAGAAALASGSQGDREPVYRDCVLQCEEQNCSGGALNHFRSRQPIYMSLAGWTCRDDCKYECMWVTVGLYLQEGHKVPQFHGKWPFSRFLFFQEPASAVASFLNGLASLVMLCRYRTFVPASSPMYHTCVAFAWVSLNAWFWSTVFHTRDTDLTEKMDYFCASTVILHSIYLCCVRTVGLQHPAVVSAFRALLLLMLTVHVSYLSLIRFDYGYNLVANVAIGLVNVVWWLAWCLWNQRRLPHVRKCVVVVLLLQGLSLLELLDFPPLFWVLDAHAIWHISTIPVHVLFFSFLEDDSLYLLKESEDKFKLD

SEQ ID NO: 41 - DNA, 2056 bp, Homo sapiens
aaagttacattttctctggaactctcctaggccactccctgctgatgcaacatctgggtttgggcagaaaggagggtgcttcggagcccgccctttctgagcttcctgggccggctctagaacaattcaggcttcgctgcgactcagacctcagctccaacatatgcattctgaagaaagatggctgagatggacagaatgctttattttggaaagaaacaatgttctaggtcaaactgagtctaccaaatgcagactttcacaatggttctagaagaaatctggacaagtcttttcatgtggtttttctacgcattgattccatgtttgctcacagatgaagtggccattctgcctgcccctcagaacctctctgtactctcaaccaacatgaagcatctcttgatgtggagcccagtgatcgcgcctggagaaacagtgtactattctgtcgaataccagggggagtacgagagcctgtacacgagccacatctggatccccagcagctggtgctcactcactgaaggtcctgagtgtgatgtcactgatgacatcacggccactgtgccatacaaccttcgtgtcagggccacattgggctcacagacctcagcctggagcatcctgaagcatccctttaatagaaactcaaccatccttacccgacctgggatggagatcaccaaagatggcttccacctggttattgagctggaggacctggggccccagtttgagttccttgtggcctactggaggagggagcctggtgccgaggaacatgtcaaaatggtgaggagtgggggtattccagtgcacctagaaaccatggagccaggggctgcatactgtgtgaaggcccagacattcgtgaaggccattgggaggtacagcgccttcagccagacagaatgtgtggaggtgcaaggagaggccattcccctggtactggccctgtttgcctttgttggcttcatgctgatccttgtggtcgtgccactgttcgtctggaaaatgggccggctgctccagtactcctgttgccccgtggtggtcctcccagacaccttgaaaataaccaattcaccccagaagttaatcagctgcagaagggaggaggtggatgcctgtgccacggctgtgatgtctcctgaggaactcctcagggcctggatctcataggtttgcggaagggcccaggtgaagccgagaacctggtctgcatgacatggaaaccatgaggggacaagttgtgtttctgttttccgccacggacaagggatgagagaagtaggaagagcctgttgtctacaagtctagaagcaaccatcagaggcagggtggtttgtctaacagaacactgactgaggcttaggggatgtgacctctagactgggggctgccacttgctggctgagcaaccctgggaaaagtgacttcatcccttcggtcctaagttttctcatctgtaatgggggaattacctacacacctgctaaacacacacacacagagtctctctctatatatacacacgtacacataaatacacccagcacttgcaaggctagagggaaactggtgacactctacagtctgactgattcagtgtttctggagagcaggacataaatgtatgatgagaatgatcaaggactctacacactgggtggcttggagagcccactttcccagaataatccttgagagaaaaggaatcatgggagcaatggtgttgagttcacttcaagcccaatgccggtgcagaggggaatggcttagcgagctctacagtaggtgacctggaggaaggtcacagccacactgaaaatgggatgtgcatgaacacggaggatccatgaactactgtaaagtgttgacagtgtgtgcacactgcagacagcaggtgaaatgtatgtgtgcaatgcgacgagaatgcagaagtcagtaacatgtgcatgtttgttgtgctccttttttctgttggtaaagtacagaattcagcaaataaaaagggccaccctggccaaaagcggtaaaaaaaaaaaaaaaa

SEQ ID NO: 42 - protein, 311 aa, Homo sapiens
MQTFTMVLEEIWTSLFMWFFYALIPCLLTDEVAILPAPQNLSVLSTNMKHLLMWSPVIAPGETVYYSVEYQGEYESLYTSHIWIPSSWCSLTEGPECDVTDDITATVPYNLRVRATLGSQTSAWSILKHPFNRNSTILTRPGMEITKDGFHLVIELEDLGPQFEFLVAYWRREPGAEEHVKMVRSGGIPVHLETMEPGAAYCVKAQTFVKAIGRYSAFSQTECVEVQGEAIPLVLALFAFVGFMLILVVVPLFVWKMGRLLQYSCCPVVVLPDTLKITNSPQKLISCRREEVDACATAVMSPEELLRAWIS

SEQ ID NO: 43 - DNA, 1660 bp, Homo sapiens
gtttgaattccttcaactatacccacagtccaaaagcagactcactgtgtcccaggctaccagttcctccaagcaagtcatttcccttatttaaccgatgtgtccctcaaacacctgagtgctactccctatttgcatctgttttgataaatgatgttgacaccctccaccgaattctaagtggaatcatgtcgggaagagatacaatccttggcctgtgtatcctcgcattagccttgtctttggccatgatgtttaccttcagattcatcaccacccttctggttcacattttcatttcattggttattttgggattgttgtttgtctgcggtgttttatggtggctgtattatgactataccaacgacctcagcatagaattggacacagaaagggaaaatatgaagtgcgtgctggggtttgctatcgtatccacaggcatcacggcagtgctgctcgtcttgatttttgttctcagaaagagaataaaattgacagttgagcttttccaaatcacaaataaagccatcagcagtgctcccttcctgctgttccagccactgtggacatttgccatcctcattttcttctgggtcctctgggtggctgtgctgctgagcctgggaactgcaggagctgcccaggttatggaaggcggccaagtggaatataagcccctttcgggcattcggtacatgtggtcgtaccatttaattggcctcatctggactagtgaattcatccttgcgtgccagcaaatgactatagctggggcagtggttacttgttatttcaacagaagtaaaaatgatcctcctgatcatcccatcctttcgtctctctccattctcttcttctaccatcaaggaaccgttgtgaaagggtcatttttaatctctgtggtgaggattccgagaatcattgtcatgtacatgcaaaacgcactgaaagaacagcagcatggtgcattgtccaggtacctgttccgatgctgctactgctgtttctggtgtcttgacaaatacctgctccatctcaaccagaatgcatatactacaactgctattaatgggacagatttctgtacatcagcaaaagatgcattcaaaatcttgtccaagaactcaagtcactttacatctattaactgctttggagacttcataatttttctaggaaaggtgttagtggtgtgtttcactgtttttggaggactcatggcttttaactacaatcgggcattccaggtgtgggcagtccctctgttattggtagctttttttgcctacttagtagcccatagttttttatctgtgtttgaaactgtgctggatgcacttttcctgtgttttgctgttgatctggaaacaaatgatggatcgtcagaaaagccctactttatggatcaagaatttctgagtttcgtaaaaaggagcaacaaattaaacaatgcaagggcacagcaggacaagcactcattaaggaatgaggagggaacagaactccaggccattgtgagatagatacccatttaggtatctgtacctggaaaacatttccttctaagagccatttacagaatagaagatgagaccactagagaaaagttagtgaatttttttttaaaagacctaataaaccctattcttcctcaaaa

SEQ ID NO: 44 - protein, 445 aa, Homo sapiens
MSGRDTILGLCILALALSLAMMFTFRFITTLLVHIFISLVILGLLFVCGVLWWLYYDYTNDLSIELDTERENMKCVLGFAIVSTGITAVLLVLIFVLRKRIKLTVELFQITNKAISSAPFLLFQPLWTFAILIFFWVLWVAVLLSLGTAGAAQVMEGGQVEYKPLSGIRYMWSYHLIGLIWTSEFILACQQMTIAGAVVTCYFNRSKNDPPDHPILSSLSILFFYHQGTVVKGSFLISVVRIPRIIVMYMQNALKEQQHGALSRYLFRCCYCCFWCLDKYLLHLNQNAYTTTAINGTDFCTSAKDAFKILSKNSSHFTSINCFGDFIIFLGKVLVVCFTVFGGLMAFNYNRAFQVWAVPLLLVAFFAYLVAHSFLSVFETVLDALFLCFAVDLETNDGSSEKPYFMDQEFLSFVKRSNKLNNARAQQDKHSLRNEEGTELQAIVR

SEQ ID NO: 45 - DNA, 2475 bp, Homo sapiens
gaggatttgccacagcagcggatagagcaggagagcaccaccggagcccttgagacatccttgagaagagccacagcataagagactgccctgcttggtgttttgcaggatgatggtggcccttcgaggagcttctgcattgctggttctgttccttgcagcttttctgcccccgccgcagtgtacccaggacccagccatggtgcattacatctaccagcgctttcgagtcttggagcaagggctggaaaaatgtacccaagcaacgagggcatacattcaagaattccaagagttctcaaaaaatatatctgtcatgctgggaagatgtcagacctacacaagtgagtacaagagtgcagtgggtaacttggcactgagagttgaacgtgcccaacgggagattgactacatacaataccttcgagaggctgacgagtgcatcgtatcagaggacaagacactggcagaaatgttgctccaagaagctgaagaagagaaaaagatccggactctgctgaatgcaagctgtgacaacatgctgatgggcataaagtctttgaaaatagtgaagaagatgatggacacacatggctcttggatgaaagatgctgtctataactctccaaaggtgtacttattaattggatccagaaacaacactgtttgggaatttgcaaacatacgggcattcatggaggataacaccaagccagctccccggaagcaaatcctaacactttcctggcagggaacaggccaagtgatctacaaaggttttctattttttcataaccaagcaacttctaatgagataatcaaatataacctgcagaagaggactgtggaagatcgaatgctgctcccaggaggggtaggccgagcattggtttaccagcactccccctcaacttacattgacctggctgtggatgagcatgggctctgggccatccactctgggccaggcacccatagccatttggttctcacaaagattgagccgggcacactgggagtggagcattcatgggataccccatgcagaagccaggatgctgaagcctcattcctcttgtgtggggttctctatgtggtctacagtactgggggccagggccctcatcgcatcacctgcatctatgatccactgggcactatcagtgaggaggacttgcccaacttgttcttccccaagagaccaagaagtcactccatgatccattacaaccccagagataagcagctctatgcctggaatgaaggaaaccagatcatttacaaactccagacaaagagaaagctgcctctgaagtaatgcattacagctgtgagaaagagcactgtggctttggcagctgttctacaggacagtgaggctatagccccttcacaatatagtatccctctaatcacacacaggaagagtgtgtagaagtggaaatacgtatgcctcctttcccaaatgtcactgccttaggtatcttccaagagcttagatgagagcatatcatcaggaaagtttcaacaatgtccattactcccccaaacctcctggctctcaaggatgaccacattctgatacagcctacttcaagccttttgttttactgctccccagcatttactgtaactctgccatcttccctcccacaattagagttgtatgccagcccctaatattcaccactggcttttctctcccctggcctttgctgaagctcttccctctttttcaaatgtctattgatattctcccattttcactgcccaactaaaatactattaatatttctttcttttcttttcttttttttgagacaaggtctcactatgttgcccaggctggtctcaaactccagagctcaagagatcctcctgcctcagcctcctaagtacctgggattacaggcatgtgccaccacacctggcttaaaatactatttcttattgaggtttaacctctatttcccctagccctgtccttccactaagcttggtagatgtaataataaagtgaaaatattaacatttgaatatcgctttccaggtgtggagtgtttgcacatcattgaattctcgtttcacctttgtgaaacatgcacaagtctttacagctgtcattctagagtttaggtgagtaacacaattacaaagtgaaagatacagctagaaaatactacaaatcccatagtttttccattgcccaaggaagcatcaaatacgtatgtttgttcacctactcttatagtcaatgcgttcatcgtttcagcctaaaaataatagtctgtccctttagccagttttcatgtctgcacaagacctttcaataggcctttcaaatgataattcctccagaaaaccagtctaagggtgaggaccccaactctagcctcctcttgtcttgctgtcctctgtttctctctttctgctttaaattcaataaaagtgacactgagcaaaaaaaaaaaaaaa

SEQ ID NO: 46 - protein, 402 aa, Homo sapiens
MMVALRGASALLVLFLAAFLPPPQCTQDPAMVHYIYQRFRVLEQGLEKCTQATRAYIQEFQEFSKNISVMLGRCQTYTSEYKSAVGNLALRVERAQREIDYIQYLREADECIVSEDKTLAEMLLQEAEEEKKIRTLLNASCDNMLMGIKSLKIVKKMMDTHGSWMKDAVYNSPKVYLLIGSRNNTVWEFANIRAFMEDNTKPAPRKQILTLSWQGTGQVIYKGFLFFHNQATSNEIIKYNLQKRTVEDRMLLPGGVGRALVYQHSPSTYIDLAVDEHGLWAIHSGPGTHSHLVLTKIEPGTLGVEHSWDTPCRSQDAEASFLLCGVLYVVYSTGGQGPHRITCIYDPLGTISEEDLPNLFFPKRPRSHSMIHYNPRDKQLYAWNEGNQIIYKLQTKRKLPLK

SEQ ID NO: 47 - DNA, 2819 bp, Homo sapiens
ctgcaagttgttaacgcctaacacacaagtatgttaggcttccaccaaagtcctcaatatacctgaatacgcacaatatcttaactcttcatatttggttttgggatctgctttgaggtcccatcttcatttaaaaaaaaatacagagacctacctacccgtacgcatacatacatatgtgtatatatatgtaaactagacaaagatcgcagatcataaagcaagctctgctttagtttccaagaagattacaaagaatttagagatgtatttgtcaagatccctgtcgattcatgccctttgggttacggtgtcctcagtgatgcagccctaccctttggtttggggacattatgatttgtgtaagactcagatttacacggaagaagggaaagtttgggattacatggcctgccagccggaatccacggacatgacaaaatatctgaaagtgaaactcgatcctccggatattacctgtggagaccctcctgagacgttctgtgcaatgggcaatccctacatgtgcaataatgagtgtgatgcgagtacccctgagctggcacacccccctgagctgatgtttgattttgaaggaagacatccctccacattttggcagtctgccacttggaaggagtatcccaagcctctccaggttaacatcactctgtcttggagcaaaaccattgagctaacagacaacatagttattacctttgaatctgggcgtccagaccaaatgatcctggagaagtctctcgattatggacgaacatggcagccctatcagtattatgccacagactgcttagatgcttttcacatggatcctaaatccgtgaaggatttatcacagcatacggtcttagaaatcatttgcacagaagagtactcaacagggtatacaacaaatagcaaaataatccactttgaaatcaaagacaggttcgcgctttttgctggacctcgcctacgcaatatggcttccctctacggacagctggatacaaccaagaaactcagagatttctttacagtcacagacctgaggataaggctgttaagaccagccgttggggaaatatttgtagatgagctacacttggcacgctacttttacgcgatctcagacataaaggtgcgaggaaggtgcaagtgtaatctccatgccactgtatgtgtgtatgacaacagcaaattgacatgcgaatgtgagcacaacactacaggtccagactgtgggaaatgcaagaagaattatcagggccgaccttggagtccaggctcctatctccccatccccaaaggcactgcaaatacctgtatccccagtatttccagtattggtacgaatgtctgcgacaacgagctcctgcactgccagaacggagggacgtgccacaacaacgtgcgctgcctgtgcccggccgcatacacgggcatcctctgcgagaagctgcggtgcgaggaggctggcagctgcggctccgactctggccagggcgcgcccccgcacggcaccccagcgctgctgctgctgaccacgctgctgggaaccgccagccccctggtgttctaggtgtcacctccagccacaccggacgggcctgtgccgtggggaagcagacacaacccaaacatttgctactaacataggaaacacacacatacagacacccccactcagacagtgtacaaactaagaaggcctaactgaactaagccatatttatcacccgtggacagcacatccgagtcaagactgttaatttctgactccagaggagttggcagctgttgatattatcactgcaaatcacattgccagctgcagagcatattgtggattggaaaggctgcgacagccccccaaacaggaaagacaaaaaacaaacaaatcaaccgacctaaaaacattggctactctagcgtggtgcgccctagtacgactccgcccagtgtgtggaccaaccaaatagcattctttgctgtcaggtgcattgtgggcataaggaaatctgttacaagctgccatattggcctgcttccgtccctgaatcccttccaacctgtgctttagtgaacgttgctctgtaaccctcgttggttgaaagatttctttgtctgatgttagtgatgcacatgtgtaacagccccctctaaaagcgcaagccagtcatacccctgtatatcttagcagcactgagtccagtgcgagcacacacccactatacaagagtggctataggaaaaaagaaagtgtatctatccttttgtattcaaatgaagttatttttcttgaactactgtaatatgtagattttttgtattattgccaatttgtgttaccagacaatctgttaatgtatctaattcgaatcagcaaagactgacattttattttgtcctctttcgttctgttttgtttcactgtgcagagatttctctgtaagggcaacgaacgtgctggcatcaaagaatatcagtttacatatataacaagtgtaataagattccaccaaaggacattctaaatgttttcttgttgctttaacactggaagatttaaagaataaaaactcctgcataaacgatttcaggaatttgtattgcaatttcttaagatgaaaggaacagccaccaagcagtttcacactcactttactgatttctgtgtggactgagtacattcagctgacgaatttagttcccaggaagatggattgatgttcactagcttggacaacttctgcaaaatatgagactatttccacttgggaaaaattacaacagcaaaaaaaaaaaaaaaaaaaaaa

SEQ ID NO: 48 - protein, 438 aa, Homo sapiens [sequence omitted]

SEQ ID NO: 49 - DNA, 3226 bp, Homo sapiens
gggggagctaggccggcggcagtggtggtggcggcggcgcaagggtgagggcggccccagaaccccaggtaggtagagcaagaagatggtgtttctgcccctcaaatggtcccttgcaaccatgtcatttctactttcctcactgttggctctcttaactgtgtccactccttcatggtgtcagagcactgaagcatctccaaaacgtagtgatgggacaccatttccttggaataaaatacgacttcctgagtacgtcatcccagttcattatgatctcttgatccatgcaaaccttaccacgctgaccttctggggaaccacgaaagtagaaatcacagccagtcagcccaccagcaccatcatcctgcatagtcaccacctgcagatatctagggccaccctcaggaagggagctggagagaggctatcggaagaacccctgcaggtcctggaacacccccctcaggagcaaattgcactgctggctcccgagcccctccttgtcgggctcccgtacacagttgtcattcactatgctggcaatctttcggagactttccacggattttacaaaagcacctacagaaccaaggaaggggaactgaggatactagcatcaacacaatttgaacccactgcagctagaatggcctttccctgctttgatgaacctgccttcaaagcaagtttctcaatcaaaattagaagagagccaaggcacctagccatctccaatatgccattggtgaaatctgtgactgttgctgaaggactcatagaagaccattttgatgtcactgtgaagatgagcacctatctggtggccttcatcatttcagattttgagtctgtcagcaagataaccaagagtggagtcaaggtttctgtttatgctgtgccagacaagataaatcaagcagattatgcactggatgctgcggtgactcttctagaattttatgaggattatttcagcataccgtatcccctacccaaacaagatcttgctgctattcccgactttcagtctggtgctatggaaaactggggactgacaacatatagagaatctgctctgttgtttgatgcagaaaagtcttctgcatcaagtaagcttggcatcacagtgactgtggcccatgaactggcccaccagtggtttgggaacctggtcactatggaatggtggaatgatctttggctaaatgaaggatttgccaaatttatggagtttgtgtctgtcagtgtgacccatcctgaactgaaagttggagattatttctttggcaaatgttttgacgcaatggaggtagatgctttaaattcctcacaccctgtgtctacacctgtggaaaatcctgctcagatccgggagatgtttgatgatgtttcttatgataagggagcttgtattctgaatatgctaagggagtatcttagcgctgacgcatttaaaagtggtattgtacagtatctccagaagcatagctataaaaatacaaaaaacgaggacctgtgggatagtatggcaagtatttgccctacagatggtgtaaaagggatggatggcttttgctctagaagtcaacattcatcttcatcctcacattggcatcaggaaggggtggatgtgaaaaccatgatgaacacttggacactgcagaggggttttcccctaataaccatcacagtgagggggaggaatgtacacatgaagcaagagcactacatgaagggctctgacggcgccccggacactgggtacctgtggcatgttccattgacattcatcaccagcaaatccaacatggtccatcgatttttgctaaaaacaaaaacagatgtgctcatcctcccagaagaggtggaatggatcaaatttaatgtgggcatgaatggctattacattgtgcattacgaggatgatggatgggactctttgactggccttttaaaaggaacacacacagcagtcagcagtaatgatcgggcaagtctcattaacaatgcatttcagctcgtcagcattgggaagctgtccattgaaaaggccttggatttatccctgtacttgaaacatgaaactgaaattatgcccgtgtttcaaggtttgaatgagctgattcctatgtataagttaatggagaaaagagatatgaatgaagtggaaactcaattcaaggccttcctcatcaggctgctaagggacctcattgataagcagacatggacagacgagggctcagtctcagagcaaatgctgcggagtgaactactactcctcgcctgtgtgcacaactatcagccgtgcgtacagagggcagaaggctatttcagaaagtggaaggaatccaatggaaacttgagcctgcctgtcgacgtgaccttggcagtgtttgctgtgggggcccagagcacagaaggctgggattttctttatagtaaatatcagttttctttgtccagtactgagaaaagccaaattgaatttgccctctgcagaacccaaaataaggaaaagcttcaatggctactagatgaaagctttaagggagataaaataaaaactcaggagtttccacaaattcttacactcattggcaggaacccagtaggatacccactggcctggcaatttctgaggaaaaactggaacaaacttgtacaaaagtttgaacttggctcatcttccatagcccacatggtaatgggtacaacaaatcaattctccacaagaacacggcttgaagaggtaaaaggattcttcagctctttgaaagaaaatggttctcagctccgttgtgtccaacagacaattgaaaccattgaagaaaacatcggttggatggataagaattttgataaaatcagagtgtggctgcaaagtgaaaagcttgaacgtatgtaaaaattcctcccttgcccggttcctgttatctctaatcaccaacattttgttgagtgtattttcaaactagagatggctgttttggctccaactggagatacttttttcccttcaactcattttttgactatccctgtgaaaagaatagctgttagtttttcatgaatgggctttttcatgaatgggctatcgctaccatgtgttttgttcatcacaggtgttgccctgcaacgtaaacccaagtgttgggttccctgccacagaagaataaagtaccttattcttctcaaaaaaaaaaaaaaaaaaaaaaaaaaa

SEQ ID NO: 50 - protein, 941 aa, Homo sapiens [sequence omitted]

SEQ ID NO: 51 - DNA, 893 bp, Homo sapiens
gtcatgccagtgcctgctctgtgcctgctctgggccctggcaatggtgacccggcctgcctcagcggcccccatgggcggcccagaactggcacagcatgaggagctgaccctgctcttccatgggaccctgcagctgggccaggccctcaacggtgtgtacaggaccacggagggacggctgacaaaggccaggaacagcctgggtctctatggccgcacaatagaactcctggggcaggaggtcagccggggccgggatgcagcccaggaacttcgggcaagcctgttggagactcagatggaggaggatattctgcagctgcaggcagaggccacagctgaggtgctgggggaggtggcccaggcacagaaggtgctacgggacagcgtgcagcggctagaagtccagctgaggagcgcctggctgggccctgcctaccgagaatttgaggtcttaaaggctcacgctgacaagcagagccacatcctatgggccctcacaggccacgtgcagcggcagaggcgggagatggtggcacagcagcatcggctgcgacagatccaggagagactccacacagcggcgctcccagcctgaatctgcctggatggaactgaggaccaatcatgctgcaaggaacacttccacgccccgtgaggcccctgtgcagggaggagctgcctgttcactgggatcagccagggcgccgggccccacttctgagcacagagcagagacagacgcaggcggggacaaaggcagaggatgtagccccattggggaggggtggaggaaggacatgtaccctttcatgcctacacacccctcattaaagcagagtcgtggcatttcaaaaaaaaaaaaaaaaaaaaaaaaaaaaaaaaaaaaaa

SEQ ID NO: 52 - protein, 198 aa, Homo sapiens
MPVPALCLLWALAMVTRPASAAPMGGPELAQHEELTLLFHGTLQLGQALNGVYRTTEGRLTKARNSLGLYGRTIELLGQEVSRGRDAAQELRASLLETQMEEDILQLQAEATAEVLGEVAQAQKVLRDSVQRLEVQLRSAWLGPAYREFEVLKAHADKQSHILWALTGHVQRQRREMVAQQHRLRQIQERLHTAALPA

SEQ ID NO: 53 - DNA, 683 bp, Homo sapiens
gcgtggggatgtctaggagctcgaaggtggtgctgggcctctcggtgctgctgacggcggccacagtggccggcgtacatgtgaagcagcagtgggaccagcagaggcttcgtgacggagttatcagagacattgagaggcaaattcggaaaaaagaaaacattcgtcttttgggagaacagattattttgactgagcaacttgaagcagaaagagagaagatgttattggcaaaaggatctcaaaaatcatgacttgaatgtgaaatatctgttggacagacaacacgagtttgtgtgtgtgtgttgatggagagtagcttagtagtatcttcatctttttttttggtcactgtccttttaaacttgatcaaataaaggacagtgggtcatataagttactgctttcagggtcccttatatctgaataaaggagtgtgggcagacactttttggaagagtctgtctgggtgatcctggtagaagccccattagggtcactgtccagtgcttagggttgttactgagaagcactgccgagcttgtgagaaggaagggatggatagtagcatccacctgagtagtctgatcagtcggcatgatgacgaagccacgagaacatcgacctcagaaggactggaggaaggtgaagtggagggagagacgctcctgatcgtcgaatcc

SEQ ID NO: 54 - protein, 81 aa, Homo sapiens
MSRSSKVVLGLSVLLTAATVAGVHVKQQWDQQRLRDGVIRDIERQIRKKENIRLLGEQIILTEQLEAEREKMLLAKGSQKS

SEQ ID NO: 55 - DNA, 3877 bp, Homo sapiens
ctccttaggtggaaaccctgggagtagagtactgacagcaaagaccgggaaagaccatacgtccccgggcaggggtgacaacaggtgtcatctttttgatctcgtgtgtggctgccttcctatttcaaggaaagacgccaaggtaattttgacccagaggagcaatgatgtagccacctcctaaccttcccttcttgaacccccagttatgccaggatttactagagagtgtcaactcaaccagcaagcggctccttcggcttaacttgtggttggaggagagaacctttgtggggctgcgttctcttagcagtgctcagaagtgacttgcctgagggtggaccagaagaaaggaaaggtcccctcttgctgttggctgcacatcaggaaggctgtgatgggaatgaaggtgaaaacttggagatttcacttcagtcattgcttctgcctgcaagatcatcctttaaaagtagagaagctgctctgtgtggtggttaactccaagaggcagaactcgttctagaaggaaatggatgcaagcagctccgggggccccaaacgcatgcttcctgtggtctagcccagggaagcccttccgtgggggccccggctttgagggatgccaccggttctggacgcatggctgattcctgaatgatgatggttcgccgggggctgcttgcgtggatttcccgggtggtggttttgctggtgctcctctgctgtgctatctctgtcctgtacatgttggcctgcaccccaaaaggtgacgaggagcagctggcactgcccagggccaacagccccacggggaaggaggggtaccaggccgtccttcaggagtgggaggagcagcaccgcaactacgtgagcagcctgaagcggcagatcgcacagctcaaggaggagctgcaggagaggagtgagcagctcaggaatgggcagtaccaagccagcgatgctgctggcctgggtctggacaggagccccccagagaaaacccaggccgacctcctggccttcctgcactcgcaggtggacaaggcagaggtgaatgctggcgtcaagctggccacagagtatgcagcagtgcctttcgatagctttactctacagaaggtgtaccagctggagactggccttacccgccaccccgaggagaagcctgtgaggaaggacaagcgggatgagttggtggaagccattgaatcagccttggagaccctgaacaatcctgcagagaacagccccaatcaccgtccttacacggcctctgatttcatagaagggatctaccgaacagaaagggacaaagggacattgtatgagctcaccttcaaaggggaccacaaacacgaattcaaacggctcatcttatttcgaccattcagccccatcatgaaagtgaaaaatgaaaagctcaacatggccaacacgcttatcaatgttatcgtgcctctagcaaaaagggtggacaagttccggcagttcatgcagaatttcagggagatgtgcattgagcaggatgggagagtccatctcactgttgtttactttgggaaagaagaaataaatgaagtcaaaggaatacttgaaaacacttccaaagctgccaacttcaggaactttaccttcatccagctgaatggagaattttctcggggaaagggacttgatgttggagcccgcttctggaagggaagcaacgtccttctctttttctgtgatgtggacatctacttcacatctgaattcctcaatacgtgtaggctgaatacacagccagggaagaaggtattttatccagttcttttcagtcagtacaatcctggcataatatacggccaccatgatgcagtccctcccttggaacagcagctggtcataaagaaggaaactggattttggagagactttggatttgggatgacgtgtcagtatcggtcagacttcatcaatataggtgggtttgatctggacatcaaaggctggggcggagaggatgtgcacctttatcgcaagtatctccacagcaacctcatagtggtacggacgcctgtgcgaggactcttccacctctggcatgagaagcgctgcatggacgagctgacccccgagcagtacaagatgtgcatgcagtccaaggccatgaacgaggcatcccacggccagctgggcatgctggtgttcaggcacgagatagaggctcaccttcgcaaacagaaacagaagacaagtagcaaaaaaacatgaactcccagagaaggattgtgggagacactttttctttccttttgcaattactgaaagtggctgcaacagagaaaagacttccataaaggacgacaaaagaattggactgatgggtcagagatgagaaagcctccgatttctctctgttgggctttttacaacagaaatcaaaatctccgctttgcctgcaaaagtaacccagttgcaccctgtgaagtgtctgacaaaggcagaatgcttgtgagattataagcctaatggtgtggaggttttgatggtgtttacaatacactgagacctgttgttttgtgtgctcattgaaatattcatgatttaagagcagttttgtaaaaaattcattagcatgaaaggcaagcatatttctcctcatatgaatgagcctatcagcagggctctagtttctaggaatgctaaaatatcagaaggcaggagaggagataggcttattatgatactagtgagtacattaagtaaaataaaatggaccagaaaagaaaagaaaccataaatatcgtgtcatattttccccaagattaaccaaaaataatctgcttatctttttggttgtccttttaactgtctccgtttttttcttttatttaaaaatgcactttttttcccttgtgagttatagtctgcttatttaattaccactttgcaagccttacaagagagcacaagttggcctacatttttatattttttaagaagatactttgagatgcattatgagaactttcagttcaaagcatcaaattgatgccatatccaaggacatgccaaatgctgattctgtcaggcactgaatgtcaggcattgagacatagggaaggaatggtttgtactaatacagacgtacagatactttctctgaagagtattttcgaagaggagcaactgaacactggaggaaaagaaaatgacactttctgctttacagaaaaggaaactcattcagactggtgatatcgtgatgtacctaaaagtcagaaaccacattttctcctcagaagtagggaccgctttcttacctgtttaaataaaccaaagtataccgtgtgaaccaaacaatctcttttcaaaacagggtgctcctcctggcttctggcttccataagaagaaatggagaaaaatatatatatatatatatatattgtgaaagatcaatccatctgccagaatctagtgggatggaagtttttgctacatgttatccaccccaggccaggtggaagtaactgaattattttttaaattaagcagttctactcaatcaccaagatgcttctgaaaattgcattttattaccatttcaaactattttttaaaaataaatacagttaacatagagtggtttcttcattcatgtgaaaattattagccagcaccagatgcatgagctaattatctctttgagtccttgcttctgtttgctcacagtaaactcattgtttaaaagcttcaagaacattcaagctgttggtgtgttaaaaaatgcattgtattgatttgtactggtagtttatgaaatttaattaaaacacaggccatgaatggaaggtggtattgcacagctaataaaatatgatttgtggatatgaa

SEQ ID NO: 56 - protein, 532 aa, Homo sapiens
MMMVRRGLLAWISRVVVLLVLLCCAISVLYMLACTPKGDEEQLALPRANSPTGKEGYQAVLQEWEEQHRNYVSSLKRQIAQLKEELQERSEQLRNGQYQASDAAGLGLDRSPPEKTQADLLAFLHSQVDKAEVNAGVKLATEYAAVPFDSFTLQKVYQLETGLTRHPEEKPVRKDKRDELVEAIESALETLNNPAENSPNHRPYTASDFIEGIYRTERDKGTLYELTFKGDHKHEFKRLILFRPFSPIMKVKNEKLNMANTLINVIVPLAKRVDKFRQFMQNFREMCIEQDGRVHLTVVYFGKEEINEVKGILENTSKAANFRNFTFIQLNGEFSRGKGLDVGARFWKGSNVLLFFCDVDIYFTSEFLNTCRLNTQPGKKVFYPVLFSQYNPGIIYGHHDAVPPLEQQLVIKKETGFWRDFGFGMTCQYRSDFINIGGFDLDIKGWGGEDVHLYRKYLHSNLIVVRTPVRGLFHLWHEKRCMDELTPEQYKMCMQSKAMNEASHGQLGMLVFRHEIEAHLRKQKQKTSSKKT

SEQ ID NO: 57 - DNA, 1658 bp, Homo sapiens
ggaaggcagcggcagctccactcagccagtacccagatacgctgggaaccttccccagccatggcttccctggggcagatcctcttctggagcataattagcatcatcattattctggctggagcaattgcactcatcattggctttggtatttcagggagacactccatcacagtcactactgtcgcctcagctgggaacattggggaggatggaatcctgagctgcacttttgaacctgacatcaaactttctgatatcgtgatacaatggctgaaggaaggtgttttaggcttggtccatgagttcaaagaaggcaaagatgagctgtcggagcaggatgaaatgttcagaggccggacagcagtgtttgctgatcaagtgatagttggcaatgcctctttgcggctgaaaaacgtgcaactcacagatgctggcacctacaaatgttatatcatcacttctaaaggcaaggggaatgctaaccttgagtataaaactggagccttcagcatgccggaagtgaatgtggactataatgccagctcagagaccttgcggtgtgaggctccccgatggttcccccagcccacagtggtctgggcatcccaagttgaccagggagccaacttctcggaagtctccaataccagctttgagctgaactctgagaatgtgaccatgaaggttgtgtctgtgctctacaatgttacgatcaacaacacatactcctgtatgattgaaaatgacattgccaaagcaacaggggatatcaaagtgacagaatcggagatcaaaaggcggagtcacctacagctgctaaactcaaaggcttctctgtgtgtctcttctttctttgccatcagctgggcacttctgcctctcagcccttacctgatgctaaaataatgtgccttggccacaaaaaagcatgcaaagtcattgttacaacagggatctacagaactatttcaccaccagatatgacctagttttatatttctgggaggaaatgaattcatatctagaagtctggagtgagcaaacaagagcaagaaacaaaaagaagccaaaagcagaaggctccaatatgaacaagataaatctatcttcaaagacatattagaagttgggaaaataattcatgtgaactagacaagtgtgttaagagtgataagtaaaatgcacgtggagacaagtgcatccccagatctcagggacctccccctgcctgtcacctggggagtgagaggacaggatagtgcatgttctttgtctctgaatttttagttatatgtgctgtaatgttgctctgaggaagcccctggaaagtctatcccaacatatccacatcttatattccacaaattaagctgtagtatgtaccctaagacgctgctaattgactgccacttcgcaactcaggggcggctgcattttagtaatgggtcaaatgattcactttttatgatgcttccaaaggtgccttggcttctcttcccaactgacaaatgccaaagttgagaaaaatgatcataattttagcataaacagagcagtcggggacaccgattttataaataaactgagcaccttctttttaaacaaaaaaaaaaaaaaaaaaaaaaaaaaaaaaaaaaaaaaaaaaaaaaaaaaaa

SEQ ID NO: 58 - protein, 282 aa, Homo sapiens
MASLGQILFWSIISIIIILAGAIALIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

SEQ ID NO: 59 - DNA, 1989 bp, Homo sapiens
gccgagtgggacaaagcctggggctgggcgggggccatggcgctgccatcccgaatcctgctttggaaacttgtgcttctgcagagctctgctgttctcctgcactcagcggtggaggagacggacgcggggctgtacacctgcaacctgcaccatcactactgccacctctacgagagcctggccgtccgcctggaggtcaccgacggccccccggccacccccgcctactgggacggcgagaaggaggtgctggcggtggcgcgcggcgcacccgcgcttctgacctgcgtgaaccgcgggcacgtgtggaccgaccggcacgtggaggaggctcaacaggtggtgcactgggaccggcagccgcccggggtcccgcacgaccgcgcggaccgcctgctggacctctacgcgtcgggcgagcgccgcgcctacgggcccctttttctgcgcgaccgcgtggctgtgggcgcggatgcctttgagcgcggtgacttctcactgcgtatcgagccgctggaggtcgccgacgagggcacctactcctgccacctgcaccaccattactgtggcctgcacgaacgccgcgtcttccacctgacggtcgccgaaccccacgcggagccgcccccccggggctctccgggcaacggctccagccacagcggcgccccaggcccagaccccacactggcgcgcggccacaacgtcatcaatgtcatcgtccccgagagccgagcccacttcttccagcagctgggctacgtgctggccacgctgctgctcttcatcctgctactggtcactgtcctcctggccgcccgcaggcgccgcggaggctacgaatactcggaccagaagtcgggaaagtcaaaggggaaggatgttaacttggcggagttcgctgtggctgcaggggaccagatgctttacaggagtgaggacatccagctagattacaaaaacaacatcctgaaggagagggcggagctggcccacagccccctgcctgccaagtacatcgacctagacaaagggttccggaaggagaactgcaaatagggaggccctgggctcctggctgggccagcagctgcacctctcctgtctgtgctcctcggggcatctcctgatgctccggggctcaccccccttccagcggctggtcccgctttcctggaatttggcctgggcgtatgcagaggccgcctccacacccctcccccaggggcttggtggcagcatagcccccacccctgcggcctttgctcacgggtggccctgcccacccctggcacaaccaaaatcccactgatgcccatcatgccctcagacccttctgggctctgcccgctgggggcctgaagacattcctggaggacactcccatcagaacctggcagccccaaaactggggtcagcctcagggcaggagtcccactcctccagggctctgctcgtccggggctgggagatgttcctggaggaggacactcccatcagaacttggcagccttgaagttggggtcagcctcggcaggagtcccactcctcctggggtgctgcctgccaccaagagctcccccacctgtaccaccatgtgggactccaggcaccatctgttctccccagggacctgctgacttgaatgccagcccttgctcctctgtgttgctttgggccacctggggctgcaccccctgccctttctctgccccatccctaccctagccttgctctcagccaccttgatagtcactgggctccctgtgacttctgaccctgacacccctcccttggactctgcctgggctggagtctagggctggggctacatttggcttctgtactggctgaggacaggggagggagtgaagttggtttggggtggcctgtgttgccactctcagcaccccacatttgcatctgctggtggacctgccaccatcacaataaagtccccatctgatttttaaaaaaaaaaaaaaa

SEQ ID NO: 60 - protein, 341 aa, Homo sapiens
MALPSRILLWKLVLLQSSAVLLHSAVEETDAGLYTCNLHHHYCHLYESLAVRLEVTDGPPATPAYWDGEKEVLAVARGAPALLTCVNRGHVWTDRHVEEAQQVVHWDRQPPGVPHDRADRLLDLYASGERRAYGPLFLRDRVAVGADAFERGDFSLRIEPLEVADEGTYSCHLHHHYCGLHERRVFHLTVAEPHAEPPPRGSPGNGSSHSGAPGPDPTLARGHNVINVIVPESRAHFFQQLGYVLATLLLFILLLVTVLLAARRRRGGYEYSDQKSGKSKGKDVNLAEFAVAAGDQMLYRSEDIQLDYKNNILKERAELAHSPLPAKYIDLDKGFRKENCK

SEQ ID NO: 61 - DNA, 3772 bp, Homo sapiens
gggggctttcttgggcttggctgcttggaacacctgcctccaaggaccggcctcggaggggtcgccgggaaagggagggaagaaggaagggcggggccggcccccctgcgcccgccccgcgcctctgcgcgcccctgtccgccccggcccagcccagcccagccccgcgggccggtcacacgcgcagccagccggccgcctcccgcgcccaagcgcgccgctctgctgtgccctgcgcccttgccccgcgccagcttctgcgcccgcagcccgcccggcgcccccggtgaccgtgaccctgccctgggcgcggggcggagcaggcatgtcccgcccggggaccgctaccccagcgctggccctggtgctcctggcagtgaccctggccggggtcggagcccagggcgcagccctcgaggaccctgattattacgggcaggagatctggagccgggagccctactacgcgcgcccggagcccgagctcgagaccttctctccgccgctgcctgcggggcccggggaggagtgggagcggcgcccgcaggagcccaggccgcccaagagggccaccaagcccaagaaagctcccaagagggagaagtcggctccggagccgcctccaccaggtaaacacagcaacaaaaaagttatgagaaccaagagctctgagaaggctgccaacgatgatcacagtgtccgtgtggcccgtgaagatgtcagagagagttgcccacctcttggtctggaaaccttaaaaatcacagacttccagctccatgcctccacggtgaagcgctatggcctgggggcacatcgagggagactcaacatccaggcgggcattaatgaaaatgatttttatgacggagcgtggtgcgcgggaagaaatgacctccagcagtggattgaagtggatgctcggcgcctgaccagattcactggtgtcatcactcaagggaggaactccctctggctgagtgactgggtgacatcctataaggtcatggtgagcaatgacagccacacgtgggtcactgttaagaatggatctggagacatgatatttgagggaaacagtgagaaggagatccctgttctcaatgagctacccgtccccatggtggcccgctacatccgcataaaccctcagtcctggtttgataatgggagcatctgcatgagaatggagatcctgggctgcccactgccagatcctaataattattatcaccgccggaacgagatgaccaccactgatgacctggattttaagcaccacaattataaggaaatgcgccagttgatgaaagttgtgaatgaaatgtgtcccaatatcaccagaatttacaacattggaaaaagccaccagggcctgaagctgtatgctgtggagatctcagatcaccctggggagcatgaagtcggtgagcccgagttccactacatcgcgggggcccacggcaatgaggtgctgggccgggagctgctgctgctgctggtgcagttcgtgtgtcaggagtacttggcccggaatgcgcgcatcgtccacctggtggaggagacgcggattcacgtcctcccctccctcaaccccgatggctacgagaaggcctacgaagggggctcggagctgggaggctggtccctgggacgctggacccacgatggaattgacatcaacaacaactttcctgatttaaacacgctgctctgggaggcagaggatcgacagaatgtccccaggaaagttcccaatcactatattgcaatccctgagtggtttctgtcggaaaatgccacggtggctgccgagaccagagcagtcatagcctggatggaaaaaatcccttttgtgctgggcggcaacctgcagggcggcgagctggtggtggcgtatccctacgacctggtgcggtccccctggaagacgcaggaacacacccccacccccgatgaccacgtgttccgctggctggcctactcctatgcctccacacaccgcctcatgacagacgcccggaggagggtgtgccacacggaggacttccagaaggaggagggcactgtcaatggggcctcctggcacaccgtcgctggaagtctgaacgatttcagctaccttcatacaaactgcttcgaactgtccatctacgtgggctgtgataaatacccacatgagagccagctgcccgaggagtgggagaataaccgggaatctctgatcgtgttcatggagcaggttcatcgtggcattaaaggcttggtgagagattcacatggaaaaggaatcccaaacgccattatctccgtagaaggcattaaccatgacatccgaacagccaacgatggggattactggcgcctcctgaaccctggagagtatgtggtcacagcaaaggccgaaggtttcactgcatccaccaagaactgtatggttggctatgacatgggggccacaaggtgtgacttcacacttagcaaaaccaacatggccaggatccgagagatcatggagaagtttgggaagcagcccgtcagcctgccagccaggcggctgaagctgcgggggcggaagagacgacagcgtgggtgaccctcctgggcccttgagactcgtctgggacccatgcaaattaaaccaacctggtagtagctccatagtggactcactcactgttgtttcctctgtaattcaagaagtgcctggaagagagggtgcattgtgaggcaggtcccaaaagggaaggctggaggctgaggctgttttcttttctttgttcccatttatccaaataacttggacagagcagcagagaaaagctgatgggagtgagagaactcagcaagccaacctgggaatcagagagagaaggagaaggaggggagcctgtccgttcagagcctctggctgcatagaaaaggattctggtgcttcccctgtttgcgtggcagcaagggttccacgtgcatttgcaatttgcacagctaaaattgcagcatttccccagctgggctgtcccaaatgttaccatttgagatgctcccaggcgtcctaagagaatccaccctctctggccctgggacattgcaagctgctacaaataaattctgtgttcttttgacaatagcgtcattgccaagtgcacatcagtgagcctcttgaatctgtttagtctcctttttcaacaaaggagtgtgttcagaaaaggagagagaggctgagatcattcaggagtttgttgggcagcaagcatggagcttcttgcacaaattctgggtccataaacaacccccaaagtccctgctgatccagtagccctggaggttccccaggtagggagagccagaggtgccagccttcctgaagggccagaaaatttagcctggatctcctcttttacctgctaggactggaaagagccagaagtggggtggcctgaagccctctctctgcttgaggtattgcccctgtgtggaattgagtgctcatgggttggcctcatatcagcctgggagttatttttgatatgtagaatgccagatcttccagattaggctaaatgtaatgaaaacctcttaggattatctgtggagcatcagtttgggaagaattattgaattatcttgcaagaaaaaagtatgtctcactttttgttaatgttgctgcctcattgacctgggaaaaatgaaaaaaaaaaataaagcaaatggtaagacccttaaaaaaaaaaaaaaaaaaaaaaaaaaaaaaaaaaaaaaaaaaaaaaaa

SEQ ID NO: 62 - protein, 756 aa, Homo sapiens
MSRPGTATPALALVLLAVTLAGVGAQGAALEDPDYYGQEIWSREPYYARPEPELETFSPPLPAGPGEEWERRPQEPRPPKRATKPKKAPKREKSAPEPPPPGKHSNKKVMRTKSSEKAANDDHSVRVAREDVRESCPPLGLETLKITDFQLHASTVKRYGLGAHRGRLNIQAGINENDFYDGAWCAGRNDLQQWIEVDARRLTRFTGVITQGRNSLWLSDWVTSYKVMVSNDSHTWVTVKNGSGDMIFEGNSEKEIPVLNELPVPMVARYIRINPQSWFDNGSICMRMEILGCPLPDPNNYYHRRNEMTTTDDLDFKHHNYKEMRQLMKVVNEMCPNITRIYNIGKSHQGLKLYAVEISDHPGEHEVGEPEFHYIAGAHGNEVLGRELLLLLVQFVCQEYLARNARIVHLVEETRIHVLPSLNPDGYEKAYEGGSELGGWSLGRWTHDGIDINNNFPDLNTLLWEAEDRQNVPRKVPNHYIAIPEWFLSENATVAAETRAVIAWMEKIPFVLGGNLQGGELVVAYPYDLVRSPWKTQEHTPTPDDHVFRWLAYSYASTHRLMTDARRRVCHTEDFQKEEGTVNGASWHTVAGSLNDFSYLHTNCFELSIYVGCDKYPHESQLPEEWENNRESLIVFMEQVHRGIKGLVRDSHGKGIPNAIISVEGINHDIRTANDGDYWRLLNPGEYVVTAKAEGFTASTKNCMVGYDMGATRCDFTLSKTNMARIREIMEKFGKQPVSLPARRLKLRGRKRRQRG

SEQ ID NO: 63 - DNA, 1346 bp, Homo sapiens
gaaagaatgttgtggctgctcttttttctggtgactgccattcatgctgaactctgtcaaccaggtgcagaaaatgcttttaaagtgagacttagtatcagaacagctctgggagataaagcatatgcctgggataccaatgaagaatacctcttcaaagcgatggtagctttctccatgagaaaagttcccaacagagaagcaacagaaatttcccatgtcctactttgcaatgtaacccagagggtatcattctggtttgtggttacagacccttcaaaaaatcacacccttcctgctgttgaggtgcaatcagccataagaatgaacaagaaccggatcaacaatgccttctttctaaatgaccaaactctggaatttttaaaaatcccttccacacttgcaccacccatggacccatctgtgcccatctggattattatatttggtgtgatattttgcatcatcatagttgcaattgcactactgattttatcagggatctggcaacgtagaagaaagaacaaagaaccatctgaagtggatgacgctgaagataagtgtgaaaacatgatcacaattgaaaatggcatcccctctgatcccctggacatgaaggggggcatattaatgatgccttcatgacagaggatgagaggctcacccctctctgaagggctgttgttctgcttcctcaagaaattaaacatttgtttctgtgtgactgctgagcatcctgaaataccaagagcagatcatatattttgtttcaccattcttcttttgtaataaattttgaatgtgcttgaaagtgaaaagcaatcaattatacccaccaacaccactgaaatcataagctattcacgactcaaaatattctaaaatatttttctgacagtatagtgtataaatgtggtcatgtggtatttgtagttattgatttaagcatttttagaaataagatcaggcatatgtatatattttcacacttcaaagacctaaggaaaaataaattttccagtggagaatacatataatatggtgtagaaatcattgaaaatggatcctttttgacgatcacttatatcactctgtatatgactaagtaaacaaaagtgagaagtaattattgtaaatggatggataaaaatggaattactcatatacagggtggaattttatcctgttatcacaccaacagttgattatatattttctgaatatcagcccctaataggacaattctatttgttgaccatttctacaatttgtaaaagtccaatctgtgctaacttaataaagtaataatcatctctttttaaaaaaaaaaaaaaaaaaaaaaaaaa

SEQ ID NO: 64 - protein, 212 aa, Homo sapiens
MLWLLFFLVTAIHAELCQPGAENAFKVRLSIRTALGDKAYAWDTNEEYLFKAMVAFSMRKVPNREATEISHVLLCNVTQRVSFWFVVTDPSKNHTLPAVEVQSAIRMNKNRINNAFFLNDQTLEFLKIPSTLAPPMDPSVPIWIIIFGVIFCIIIVAIALLILSGIWQRRRKNKEPSEVDDAEDKCENMITIENGIPSDPLDMKGGILMMPS

SEQ ID NO: 65 - DNA, 1701 bp, Homo sapiens
  Unsure 1528: Unknown base
gagactgcagagggagataaagagagagggcaaagaggcagcaagagatttgtcctggggatccagaaacccatgataccctactgaacaccgaatcccctggaagcccacagagacagagacagcaagagaagcagagataaatacactcacgccaggagctcgctcgctctctctctctctctctcactcctccctccctctctctctgcctgtcctagtcctctagtcctcaaattcccagtcccctgcaccccttcctgggacactatgttgttctccgccctcctgctggaggtgatttggatcctggctgcagatgggggtcaacactggacgtatgagggcccacatggtcaggaccattggccagcctcttaccctgagtgtggaaacaatgcccagtcgcccatcgatattcagacagacagtgtgacatttgaccctgatttgcctgctctgcagccccacggatatgaccagcctggcaccgagcctttggacctgcacaacaatggccacacagtgcaactctctctgccctctaccctgtatctgggtggacttccccgaaaatatgtagctgcccagctccacctgcactggggtcagaaaggatccccaggggggtcagaacaccagatcaacagtgaagccacatttgcagagctccacattgtacattatgactctgattcctatgacagcttgagtgaggctgctgagaggcctcagggcctggctgtcctgggcatcctaattgaggtgggtgagactaagaatatagcttatgaacacattctgagtcacttgcatgaagtcaggcataaagatcagaagacctcagtgcctcccttcaacctaagagagctgctccccaaacagctggggcagtacttccgctacaatggctcgctcacaactcccccttgctaccagagtgtgctctggacagttttttatagaaggtcccagatttcaatggaacagctggaaaagcttcaggggacattgttctccacagaagaggagccctctaagcttctggtacagaactaccgagcccttcagcctctcaatcagcgcatggtctttgcttctttcatccaagcaggatcctcgtataccacaggtgaaatgctgagtctaggtgtaggaatcttggttggctgtctctgccttctcctggctgtttatttcattgctagaaagattcggaagaagaggctggaaaaccgaaagagtgtggtcttcacctcagcacaagccacgactgaggcataaattccttctcagataccatggatgtggatgacttcccttcatgcctatcaggaagcctctaaaatggggtgtaggatctggccagaaacactgtaggagtagtaagcagatgtcctccttcccctggacatctcttagagaggaatggacccaggctgtcattccaggaagaactgcagagccttcagcctctccaaacatgtaggaggaaatgaggaaatcgctgtgttgttaatgcagagancaaactctgtttagttgcaggggaagtttgggatataccccaaagtcctctaccccctcacttttatggccctttccctagatatactgcgggatctctccttaggataaagagttgctgttgaagttgtatatttttgatcaatatatttggaaattaaagtttctgacttt

SEQ ID NO: 66 - protein, 337 aa, Homo sapiens
MLFSALLLEVIWILAADGGQHWTYEGPHGQDHWPASYPECGNNAQSPIDIQTDSVTFDPDLPALQPHGYDQPGTEPLDLHNNGHTVQLSLPSTLYLGGLPRKYVAAQLHLHWGQKGSPGGSEHQINSEATFAELHIVHYDSDSYDSLSEAAERPQGLAVLGILIEVGETKNIAYEHILSHLHEVRHKDQKTSVPPFNLRELLPKQLGQYFRYNGSLTTPPCYQSVLWTVFYRRSQISMEQLEKLQGTLFSTEEEPSKLLVQNYRALQPLNQRMVFASFIQAGSSYTTGEMLSLGVGILVGCLCLLLAVYFIARKIRKKRLENRKSVVFTSAQATTEA

SEQ ID NO: 67 - DNA, 2786 bp, Homo sapiens
ccggggacatgaggtggatactgttcattggggcccttattgggtccagcatctgtggccaagaaaaattttttggggaccaagttttgaggattaatgtcagaaatggagacgagatcagcaaattgagtcaactagtgaattcaaacaacttgaagctcaatttctggaaatctccctcctccttcaatcggcctgtggatgtcctggtcccatctgtcagtctgcaggcatttaaatccttcctgagatcccagggcttagagtacgcagtgacaattgaggacctgcaggcccttttagacaatgaagatgatgaaatgcaacacaatgaagggcaagaacggagcagtaataacttcaactacggggcttaccattccctggaagctatttaccacgagatggacaacattgccgcagactttcctgacctggcgaggagggtgaagattggacattcgtttgaaaaccggccgatgtatgtactgaagttcagcactgggaaaggcgtgaggcggccggccgtttggctgaatgcaggcatccattcccgagagtggatctcccaggccactgcaatctggacggcaaggaagattgtatctgattaccagagggatccagctatcacctccatcttggagaaaatggatattttcttgttgcctgtggccaatcctgatggatatgtgtatactcaaactcaaaaccgattatggaggaagacgcggtcccgaaatcctggaagctcctgcattggtgctgacccaaatagaaactggaacgctagttttgcaggaaagggagccagcgacaacccttgctccgaagtgtaccatggaccccacgccaattcggaagtggaggtgaaatcagtggtagatttcatccaaaaacatgggaatttcaagggcttcatcgacctgcacagctactcgcagctgctgatgtatccatatgggtactcagtcaaaaaggccccagatgccgaggaactcgacaaggtggcgaggcttgcggccaaagctctggcttctgtgtcgggcactgagtaccaagtgggtcccacctgcaccactgtctatccagctagcgggagcagcatcgactgggcgtatgacaacggcatcaaatttgcattcacatttgagttgagagataccgggacctatggcttcctcctgccagctaaccagatcatccccactgcagaggagacgtggctggggctgaagaccatcatggagcatgtgcgggacaacctctactaggcgatggctctgctctgtctacatttatttgtacccacacgtgcacgcactgaggccattgttaaaggagctctttcctacctgtgtgagtcagagccctctgggtttgtggagcacacaggcctgcccctctccagccagctccctggagtcgtgtgtcctggcggtgtccctgcaagaactggttctgccagcctgctcaattttggtcctgctgtttttgatgagccttttgtctgtttctccttccaccctgctggctgggcggctgcactcagcatcaccccttcctgggtggcatgtctctctctacctcatttttagaaccaaagaacatctgagatgattctctaccctcatccacatctagccaagccagtgaccttgctctggtggcactgtgggagacaccacttgtctttaggtgggtctcaaagatgatgtagaatttcctttaatttctcgcagtcttcctggaaaatattttcctttgagcagcaaatcttgtagggatatcagtgaaggtctctccctccctcctctcctgttttttttttttttgagacagagttttgctcttgttgcccaggctggagtgtgatggctcgatcttggctcaccacaacctctgcctcctgggttcaagcaattctcctgcctcagcctcttgagtagcttggtttataggcgcatgccaccatgcctggctaattttgtgtttttagtagagacagggtttctccatgttggtcaggctggtctcaaactcccaacctcaggtgatctgccctccttggcctcccagagtgctgggattacaggtgtgagccactgtgccgggcccgtcccctccttttttaggcctgaatacaaagtagaagatcactttccttcactgtgctgagaatttctagatactacagttcttactcctctcttccctttgttattcagtgtgaccaggatggcgggaggggatctgtgtcactgtaggtactgtgcccaggaaggctgggtgaagtgaccatctaaattgcaggatggtgaaattatccccatctgtcctaatgggcttacctcctctttgccttttgaactcacttcaaagatctaggcctcatcttacaggtcctaaatcactcatctggcctggataatctcactgccctggcacattcccatttgtgctgtggtgtatcctgtgtttccttgtcctggtttgtgtgtgtgtgtgtgtgtgtgtgtgtgtgtgtgtttgtgtgtgtgtgtctgtctattttgtatcctggaccacaagttcctaagtagagcaagaattcatcaaccagctgcctcttgtttcatttcacctcagcacgtaccatctgtccttttgttgttgttgttttgtttttgtttttttgcttttaccaaacatgtctgtaaatcttaacctcctgcctaggatttgtacagcatctggtgtgtgcttataagccaataaatattcaatgtgaaaaaaaaaaaaaaaaa

SEQ ID NO: 68 - protein, 421 aa, Homo sapiens
MRWILFIGALIGSSICGQEKFFGDQVLRINVRNGDEISKLSQLVNSNNLKLNFWKSPSSFNRPVDVLVPSVSLQAFKSFLRSQGLEYAVTIEDLQALLDNEDDEMQHNEGQERSSNNFNYGAYHSLEAIYHEMDNIAADFPDLARRVKIGHSFENRPMYVLKFSTGKGVRRPAVWLNAGIHSREWISQATAIWTARKIVSDYQRDPAITSILEKMDIFLLPVANPDGYVYTQTQNRLWRKTRSRNPGSSCIGADPNRNWNASFAGKGASDNPCSEVYHGPHANSEVEVKSVVDFIQKHGNFKGFIDLHSYSQLLMYPYGYSVKKAPDAEELDKVARLAAKALASVSGTEYQVGPTCTTVYPASGSSIDWAYDNGIKFAFTFELRDTGTYGFLLPANQIIPTAEETWLGLKTIMEHVRDNLY

SEQ ID NO: 69 - DNA, 2258 bp, Homo sapiens
aggatgtcttctggcaatttcatataagtattttttcaaaaatgtctcttctgtcaaccccacgcctttggcacaatgaagtgggtaacctttatttcccttctttttctctttagctcggcttattccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgtagctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtcagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaaatgtgccagtctccaaaaatttggagaaagagctttcaaagcatgggcagtggctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaggattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaactgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcacaaaatgctgcacagagtccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagcttgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttataacatctacatttaaaagcatctcagcctaccatgagaataagagaaagaaaatgaagatcaaaagcttattcatctgttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgctatcctgaaaattctgtaggttctgtggaagttccagtgttctctcttattccacttcggtagaggatttctagtttctgtgggctaattaaataaatcactaatactcttctaagttaaaaaaa

SEQ ID NO: 70 - protein, 609 aa, Homo sapiens [sequence omitted]

SEQ ID NO: 71 - DNA, 1475 bp, Homo sapiens
gagagaagtcagcctggcagagagactctgaaatgagggattagaggtgttcaaggagcaagagcttcagcctgaagacaagggagcagtccctgaagacgcttctactgagaggtctgccatggcctctcttggcctccaacttgtgggctacatcctaggccttctggggcttttgggcacactggttgccatgctgctccccagctggaaaacaagttcttatgtcggtgccagcattgtgacagcagttggcttctccaagggcctctggatggaatgtgccacacacagcacaggcatcacccagtgtgacatctatagcacccttctgggcctgcccgctgacatccaggctgcccaggccatgatggtgacatccagtgcaatctcctccctggcctgcattatctctgtggtgggcatgagatgcacagtcttctgccaggaatcccgagccaaagacagagtggcggtagcaggtggagtctttttcatccttggaggcctcctgggattcattcctgttgcctggaatcttcatgggatcctacgggacttctactcaccactggtgcctgacagcatgaaatttgagattggagaggctctttacttgggcattatttcttccctgttctccctgatagctggaatcatcctctgcttttcctgctcatcccagagaaatcgctccaactactacgatgcctaccaagcccaacctcttgccacaaggagctctccaaggcctggtcaacctcccaaagtcaagagtgagttcaattcctacagcctgacagggtatgtgtgaagaaccaggggccagagctggggggtggctgggtctgtgaaaaacagtggacagcaccccgagggccacaggtgagggacactaccactggatcgtgtcagaaggtgctgctgaggatagactgactttggccattggattgagcaaaggcagaaatgggggctagtgtaacagcatgcaggttgaattgccaaggatgctcgccatgccagcctttctgttttcctcaccttgctgctcccctgccctaagtccccaaccctcaacttgaaaccccattcccttaagccaggactcagaggatccctttgccctctggtttacctgggactccatccccaaacccactaatcacatcccactgactgaccctctgtgatcaaagaccctctctctggctgaggttggctcttagctcattgctggggatgggaaggagaagcagtggcttttgtgggcattgctctaacctacttctcaagcttccctccaaagaaactgattggccctggaacctccatcccactcttgttatgactccacagtgtccagactaatttgtgcatgaactgaaataaaaccatcctacggtatccagggaacagaaagcaggatgcaggatgggaggacaggaaggcagcctgggacatttaaaaaaata

SEQ ID NO: 72 - protein, 230 aa, Homo sapiens
MASLGLQLVGYILGLLGLLGTLVAMLLPSWKTSSYVGASIVTAVGFSKGLWMECATHSTGITQCDIYSTLLGLPADIQAAQAMMVTSSAISSLACIISVVGMRCTVFCQESRAKDRVAVAGGVFFILGGLLGFIPVAWNLHGILRDFYSPLVPDSMKFEIGEALYLGIISSLFSLIAGIILCFSCSSQRNRSNYYDAYQAQPLATRSSPRPGQPPKVKSEFNSYSLTGYV

SEQ ID NO: 73 - DNA, 681 bp, Homo sapiens
gcacctgcgaccaccgtgagcagtcatggcgtactccacagtgcagagagtcgctctggcttctgggcttgtcctggctctgtcgctgctgctgcccaaggccttcctgtcccgcgggaagcggcaggagccgccgccgacacctgaaggaaaattgggccgatttccacctatgatgcatcatcaccaggcaccctcagatggccagactcctggggctcgtttccagaggtctcaccttgccgaggcatttgcaaaggccaaaggatcaggtggaggtgctggaggaggaggtagtggaagaggtctgatggggcagattattccaatctacggttttgggatttttttatatatactgtacattctatttaaggtaagtagaatcatcctaatcatattacatcaatgaaaatctaatatggcgataaaaatcattgtctacattaaaacttcttatagttcataaaattatttcaaatccatcatctctttaaatcctgcctcctcttcatgaggtacttaggatagccattatttcagtttcacataagaatgtttactcaatgtttaagtgttttgccccaaaattcacaactaacaaggcagaactaggacttgaacatggatcttttggttcttaatccagtgagtgatacaattcaatgcactcccctgcca

SEQ ID NO: 74 - protein, 128 aa, Homo sapiens
MAYSTVQRVALASGLVLALSLLLPKAFLSRGKRQEPPPTPEGKLGRFPPMMHHHQAPSDGQTPGARFQRSHLAEAFAKAKGSGGGAGGGGSGRGLMGQIIPIYGFGIFLYILYILFKVSRIILIILHQ

SEQ ID NO: 75 - DNA, 2044 bp, Homo sapiens
gggggcgggtgcctggagcacggcgctggggccgcccgcagcgctcactcgctcgcactcagtcgcgggaggcttccccgcgccggccgcgtcccgcccgctccccggcaccagaagttcctctgcgcgtccgacggcgacatgggcgtccccacggccctggaggccggcagctggcgctggggatccctgctcttcgctctcttcctggctgcgtccctaggtccggtggcagccttcaaggtcgccacgccgtattccctgtatgtctgtcccgaggggcagaacgtcaccctcacctgcaggctcttgggccctgtggacaaagggcacgatgtgaccttctacaagacgtggtaccgcagctcgaggggcgaggtgcagacctgctcagagcgccggcccatccgcaacctcacgttccaggaccttcacctgcaccatggaggccaccaggctgccaacaccagccacgacctggctcagcgccacgggctggagtcggcctccgaccaccatggcaacttctccatcaccatgcgcaacctgaccctgctggatagcggcctctactgctgcctggtggtggagatcaggcaccaccactcggagcacagggtccatggtgccatggagctgcaggtgcagacaggcaaagatgcaccatccaactgtgtggtgtacccatcctcctcccaggatagtgaaaacatcacggctgcagccctggctacgggtgcctgcatcgtaggaatcctctgcctccccctcatcctgctcctggtctacaagcaaaggcaggcagcctccaaccgccgtgcccaggagctggtgcggatggacagcaacattcaagggattgaaaaccccggctttgaagcctcaccacctgcccaggggatacccgaggccaaagtcaggcaccccctgtcctatgtggcccagcggcagccttctgagtctgggcggcatctgctttcggagcccagcacccccctgtctcctccaggccccggagacgtcttcttcccatccctggaccctgtccctgactctccaaactttgaggtcatctagcccagctgggggacagtgggctgttgtggctgggtctggggcaggtgcatttgagccagggctggctctgtgagtggcctccttggcctcggccctggttccctccctcctgctctgggctcagatactgtgacatcccagaagcccagcccctcaacccctctggatgctacatggggatgctggacggctcagcccctgttccaaggattttggggtgctgagattctcccctagagacctgaaattcaccagctacagatgccaaatgacttacatcttaagaagtctcagaacgtccagcccttcagcagctctcgttctgagacatgagccttgggatgtggcagcatcagtgggacaagatggacactgggccaccctcccaggcaccagacacagggcacggtggagagacttctcccccgtggccgccttggctcccccgttttgcccgaggctgctcttctgtcagacttcctctttgtaccacagtggctctggggccaggcctgcctgcccactggccatcgccaccttccccagctgcctcctaccagcagtttctctgaagatctgtcaacaggttaagtcaatctggggcttccactgcctgcattccagtccccagagcttggtggtcccgaaacgggaagtacatattggggcatggtggcctccgtgagcaaatggtgtcttgggcaatctgaggccaggacagatgttgccccacccactggagatggtgctgagggaggtgggtggggccttctgggaaggtgagtggagaggggcacctgccccccgccctccccatcccctactcccactgctcagcgcgggccattgcaagggtgccacacaatgtcttgtccaccctgggacacttctgagtatgaagcgggatgctattaaaaactacatggggaaaaaaaaaaaaaaaaaaaaaaaaaaaaaaaaaaaaaga

SEQ ID NO: 76 - protein, 311 aa, Homo sapiens
MGVPTALEAGSWRWGSLLFALFLAASLGPVAAFKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITAAALATGACIVGILCLPLILLLVYKQRQAASNRRAQELVRMDSNIQGIENPGFEASPPAQGIPEAKVRHPLSYVAQRQPSESGRHLLSEPSTPLSPPGPGDVFFPSLDPVPDSPNFEVI

SEQ ID NO: 77 - DNA, 4563 bp, Homo sapiens
  Unsure 3635: Unknown base
ctaagccggaggatgtgcagctgcggcggcggcgccggctacgaagaggacggggacaggcgccgtgcgaaccgagcccagccagccggaggacgcgggcagggcgggacgggagcccggactcgtctgccgccgccgtcgtcgccgtcgtgccggccccgcgtccccgcgcgcgagcgggaggagccgccgccacctcgcgcccgagccgccgctagcgcgcgccgggcatggtcccctcttaaaggcgcaggccgcggcggcgggggcgggtgtgcggaacaaagcgccggcgcggggcctgcgggcggctcgggggccgcgatgggcgcggcgggcccgcggcggcggcggcgctgcccgggccgggcctcgcggcgctagggcgggctggcctccgtgggcgggggcagcgggctgagggcgcgcggagcctgcggcggcggcggcggcggcggcggcggcccggcgggcggagcggcgcgggcatggccgcgcgcggccggcgcgcctggctcagcgtgctgctcgggctcgtcctgggcttcgtgctggcctcgcggctcgtcctgccccgggcttccgagctgaagcgagcgggcccacggcgccgcgccagccccgagggctgccggtccgggcaggcggcggcttcccaggccggcggggcgcgcggcgatgcgcgcggggcgcagctctggccgcccggctcggacccagatggcggcccgcgcgacaggaactttctcttcgtgggagtcatgaccgcccagaaatacctgcagactcgggccgtggccgcctacagaacatggtccaagacaattcctgggaaagttcagttcttctcaagtgagggttctgacacatctgtaccaattccagtagtgccactacggggtgtggacgactcctacccgccccagaagaagtccttcatgatgctcaagtacatgcacgaccactacttggacaagtatgaatggtttatgagagcagatgatgacgtgtacatcaaaggagaccgtctggagaacttcctgaggagtttgaacagcagcgagcccctctttcttgggcagacaggcctgggcaccacggaagaaatgggaaaactggccctggagcctggtgagaacttctgcatgggggggcctggcgtgatcatgagccgggaggtgcttcggagaatggtgccgcacattggcaagtgtctccgggagatgtacaccacccatgaggacgtggaggtgggaaggtgtgtccggaggtttgcaggggtgcagtgtgtctggtcttatgagatgcggcagcttttttatgagaattacgagcagaacaaaaaggggtacattagagatctccataacagtaaaattcaccaagctatcacattacaccccaacaaaaacccaccctaccagtacaggctccacagctacatgctgagccgcaagatatccgagctccgccatcgcacaatacagctgcaccgcgaaattgtcctgatgagcaaatacagcaacacagaaattcataaagaggacctccagctgggaatccctccctccttcatgaggtttcagccccgccagcgagaggagattctggaatgggagtttctgactggaaaatacttgtattcggcagttgacggccagccccctcgaagaggaatggactccgcccagagggaagccttggacgacattgtcatgcaggtcatggagatgatcaatgccaacgccaagaccagagggcgcatcattgacttcaaagagatccagtacggctaccgccgggtgaaccccatgtatggggctgagtacatcctggacctgctgcttctgtacaaaaagcacaaagggaagaaaatgacggtccctgtgaggaggcacgcgtatttacagcagactttcagcaaaatccagtttgtggagcatgaggagctggatgcacaagagttggccaagagaatcaatcaggaatctggatccttgtcctttctctcaaactccctgaagaagctcgtcccctttcagctccctgggtcgaagagtgagcacaaagaacccaaagataaaaagataaacatactgattcctttgtctgggcgtttcgacatgtttgtgagatttatgggaaactttgagaagacgtgtcttatccccaatcagaacgtcaagctcgtggttctgcttttcaattctgactccaaccctgacaaggccaaacaagttgaactgatgagagattaccgcattaagtaccctaaagccgacatgcagattttgcctgtgtctggagagttttcaagagccctggccctggaagtaggatcctcccagtttaacaatgaatctttgctcttcttctgcgacgtcgacctcgtgtttactacagaattccttcagcgatgtcgagcaaatacagttctgggccaacaaatatattttccaatcatcttcagccagtatgacccaaagattgtttatagtgggaaagttcccagtgacaaccattttgcctttactcagaaaactggcttctggagaaactatgggtttggcatcacgtgtatttataagggagatcttgtccgagtgggtggctttgatgtttccatccaaggctgggggctggaggatgtggaccttttcaacaaggttgtccaggcaggtttgaagacgtttaggagccaggaagtaggagtagtccacgtccaccatcctgtcttttgtgatcccaatcttgaccccaaacagtacaaaatgtgcttggggtccaaagcatcgacctatgggtccacccagcagctggctgagatgtggctggaaaaaaatgatccaagttacagtaaaagcagcaataataatggctcagtgaggacagcctaatgtccagctttgctggaaaagacgtttttaattatctaatttatttttcaaaaattttttgtatgatcagtttttgaagtccgtatacaaggatatattttacaagtggttttcttacataggactcctttaagattgagctttctgaacaagaaggtgatcagtgtttgcctttgaacacatcttcttgctgaacattatgtagcagacctgcttaactttgacttgaaatgtacctgatgaacaaaacttttttaaaaaaatgttttcttttgagaccctttgctccagtcctatggcagaaaacgtgaacattcctgcaaagtattattgtaacaaaacactgtaactctggtaaatgttctgttgtgattgttaacattccacagattctaccttttgtgttttgttttttttttttacaattgttttaaagccatttcatgttccagttgtaagataaggaaatgtgataatagctgtttcatcattgtcttcaggagagctttccagagttgatcatttcctctcatggtactctgctcagcatggccacgtaggttttttgtttgttttgttttgttctttttttgagacggagtctcactctgttacccaggctggaatgcagtggcgcaatcttggctcactttaacctccacttccctggttcaagcaattcccctgcctttgcctcccgagtagctgggattacaggcacacaccaccacgcccagntagtttttttgtatttttagtagagacggggtttcaccatgcaagcccagctggccacgtaggttttaaagcaaggggcgtgaagaaggcacagtgaggtatgtggctgttctcgtggtagttcattcggcctaaatagacctggcattaaatttcaagaaggatttggcattttctcttcttgacccttctctttaaagggtaaaatattaatgtttagaatgacaaagatgaattattacaataaatctgatgtacacagactgaaacatacacacatacaccctaatcaaaacgttggggaaaaatgtatttggttttgttcctttcatcctgtctgtgttatgtgggtggagatggttttcattctttcattactgttttgttttatcctttgtatctgaaatacctttaatttatttaatatctgttgttcagagctctgccatttcttgagtacctgttagttagtattatttatgtgtatcgggagtgtgtttagtctgttttatttgcagtaaaccgatctccaaagatttccttttggaaacgctttttcccctccttaatttttatattccttactgttttactaaatattaagtgttctttgacaattttggtgctcatgtgttttggggacaaaagtgaaatgaatctgtcattataccagaaagttaaattctcagatcaaatgtgccttaataaatttgttttcatttagatttcaaacagtgatagacttgccattttaatacacgtcattggagggctgcgtatttgtaaatagcctgatgctcatttggaaaaataaaccagtgaacaatatttttctattgtacttttcgaaccattttgtctcattattcctgttttagctgaagaattgtattacatttggagagtaaaaaacttaaacacgaaaaaa

SEQ ID NO: 78 - protein, 802 aa, Homo sapiens [sequence omitted]

SEQ ID NO: 79 - DNA, 2134 bp, Homo sapiens
ggccgttggttggtgcgcggctgaagggtgtggcgcgagcagcgtcgttggttggccggcggcgggccgggacgggcatggccctgctgctgtgcctggtgtgcctgacggcggcgctggcccacggctgtctgcactgccacagcaacttctccaagaagttctccttctaccgccaccatgtgaacttcaagtcctggtgggtgggcgacatccccgtgtcaggggcgctgctcaccgactggagcgacgacacgatgaaggagctgcacctggccatccccgccaagatcacccgggagaagctggaccaagtggcgacagcagtgtaccagatgatggatcagctgtaccaggggaagatgtacttccccgggtatttccccaacgagctgcgaaacatcttccgggagcaggtgcacctcatccagaacgccatcatcgaaaggcacctggcaccaggcagctggggaggagggcagctctccagggagggacccagcctagcacctgaaggatcaatgccatcaccccgcggggacctcccctaagtagcccccagaggcgctgggagtgttgccaccgccctcccctgaagtttgctccatctcacgctgggggtcaacctggggaccccttccctccgggccatggacacacatacatgaaaaccaggccgcatcgactgtcagcaccgctgtggcatcttccagtacgagaccatctcctgcaacaactgcacagactcgcacgtcgcctgctttggctataactgcgagtagggctcaggcatcacacccacccgtgccagggccctactgtccctggggtcccaggctctccttggagggggctccccgccttccacctggctgtcatcgggtagggcggggccgtgggttcaggggcgcaccacttccaagcctgtgtcccacaggtcctcggcgcagtggaagtcagctgtccagggcctcctgaactacataaataactggcacaagtaagtcccctcctcaaaccaacacaggcagtgtgtgtatgtgagcacctcgtgggtgagtatgtgtggggcacaggctggctccctcagctcccacgtcctagaggggctcccgaggaggtggaacctcaacccagctctgcgcaggaggcggctgcagtccttttctccctcaaaggtctccgaccctcagctggaggcgggcatctttcctaaagggtccccatagggtctggttccaccccatcccaggtctgtggtcagagcctgggagggttccctacgatggttaggggtgccccatggaggggctgactgccccacattgcctttcagacaggacacgagcatgaggtaaggccgccctgacctggacttcagggggagggggtaaagggagagaggaggggggctagggggtcctctagatcagtgggggcactgcaggtggggctctccctatacctgggacacctgctggatgtcacctctgcaaccacacccatgtggtggtttcatgaacagaccacgctcctctgccttctcctggcctgggacacacagagccaccccggccttgtgagtgacccagagaagggaggcctcgggagaaggggtgctcgtaagccaacaccagcgtgccgcggcctgcacacccttcggacatcccaggcacgagggtgtcgtggatgtggccacacataggaccacacgtcccagctgggaggagaggcctggggcccccagggagggaggcagggggtgggggacatggagagctgaggcagcctcgtctccccgcagcctggtatcgccagccttaaggtgtctggagcccccacacttggccaacctgaccttggaagatgctgctgagtgtctcaagcagcactgacagcagctgggcctgccccagggcaacgtgggggcggagactcagctggacagcccctgcctgtcactctggagctgggctgctgctgcctcaggaccccctctccgaccccggacagagctgagctggccagggccaggagggcgggagggagggaatgggggtgggctgtgcgcagcatcagcgcctgggcaggtccgcagagctgcgggatgtgattaaagtccctgatgtttctc

SEQ ID NO: 80 - protein, 157 aa, Homo sapiens
MALLLCLVCLTAALAHGCLHCHSNFSKKFSFYRHHVNFKSWWVGDIPVSGALLTDWSDDTMKELHLAIPAKITREKLDQVATAVYQMMDQLYQGKMYFPGYFPNELRNIFREQVHLIQNAIIERHLAPGSWGGGQLSREGPSLAPEGSMPSPRGDLP

SEQ ID NO: 81 - DNA, 2550 bp, Homo sapiens
cctgagcaaacacagcagcccgagtgttcccaaggccaaaatgctgagaacgtccactcctaatctgtgtggtggtctgcattgccgggccccctggctctcttctggcattctctgcctctgcctcatattcttgttaggccaggtgggcttgctgcagggacacccccagtgcctggattacgggccccctttccagccccctctgcaccttgagttttgctctgactatgagtccttcggctgctgtgatcagcacaaggaccgccgcatcgctgcccggtactgggacatcatggaatattttgatctgaagagacatgagctgtgtggagattacattaaagacatcctttgccaggagtgctcgccctacgcagcccacctctacgacgccgaaaacacccagacgcctctccggaatctcccgggcctctgctctgattactgctctgccttccattctaactgtcactcagccatttccctgctgaccaatgaccgcggcctccaggagtctcatggaagggacggtacccgcttctgccacctcctggaccttcctgacaaggactattgcttccctaatgtcctgaggaacgactatctcaaccgccacctgggcatggtggcccaagatcctcagggctgcctgcagctctgcctgagcgaggtggccaacgggctgaggaaccccgtctccatggtccatgctggggacggcacccatcgcttctttgttgccgagcaggtaggagtggtgtgggtctacctccctgatgggagtcgcctggagcaacccttcctggacctcaagaacatcgtgttgaccaccccatggatcggggatgagagaggcttcttggggttggcttttcaccccaaattccgccacaatcgcaagttctatatttattattcgtgcctggacaagaagaaggtagaaaagatccgaattagtgagatgaaggtttctcgggctgatcctaacaaagctgacctgaaatcagagagggtcatcttggagattgaagaaccagcctcaaaccataatggcggacaacttctttttggcctggatggctatatgtacatattcactggggacgggggacaggctggagatccctttggcctgtttggaaatgctcagaacaaaagttccctgctgggaaaagttttaaggatcgatgtgaacagggcaggctcacatggcaagcggtaccgagtcccctcggacaatccatttgtttctgagccaggggcccaccccgccatctatgcctatgggatcaggaacatgtggcgttgtgctgtggaccgaggggaccccatcacgcgccagggccgaggccggatattctgtggggacgtgggccagaacaggtttgaagaggttgacctcattttgaaaggtggaaactatggctggagagcaaaggaagggtttgcatgttatgacaaaaaactttgtcacaatgcctctttggatgatgttctgccaatctatgcttatggccatgcagtggggaagtcagtcactggaggttatgtctatcgtggttgtgaatccccaaatctcaatggcctgtatatctttggagacttcatgagtggtcgacttatggctttgcaggaagatagaaaaaacaagaaatggaagaagcaggatctttgcctgggcagcaccacgtcctgtgccttcccagggctgatcagcacccatagcaagttcatcatctcctttgctgaagatgaagcaggggagctgtatttcctggcgacctcttacccaagtgcctatgcaccacgtggatctatttacaagtttgttgacccctcaaggcgagcacccccaggcaagtgcaaatacaagccagtgcccgtgagaaccaagagtaagcggatcccgttcagaccactcgccaagacagtcttggacttgctaaaggaacaatcagagaaagctgctagaaaatcttccagtgcaaccttagcttctggcccagcccagggtttgtctgagaaaggctcctccaagaagctggcttctcctacaagcagcaagaatacattgcgagggcctggtacaaagaagaaagccagagtggggccccacgtccgccagggcaagaggaggaagagcctgaaaagccacagtggcaggatgaggccatcagcagagcagaagcgagctggcagaagtctcccttgacctattggtcaaggtggccgacagggtgacgtgagagaggagagccacctcatcaaatgaaagtcactgctgaataaagaccttagaagtctgggaagccagggtagaggtggggcagggcggttttcctctccctgggaaatcttgctgtctactgaataaataaatgcaccttctctgtatgcagtgcttctgtgggagaccatatcccagattgctggtgcacctgggttatggtaagcactagtccatgagcctgcttggaatcacactggatgtctccgttttgtcttgtaaatgcctacaacctgaggtaataaatcaacatttgctca

SEQ ID NO: 82 - protein, 724 aa, Homo sapiens
MLRTSTPNLCGGLHCRAPWLSSGILCLCLIFLLGQVGLLQGHPQCLDYGPPFQPPLHLEFCSDYESFGCCDQHKDRRIAARYWDIMEYFDLKRHELCGDYIKDILCQECSPYAAHLYDAENTQTPLRNLPGLCSDYCSAFHSNCHSAISLLTNDRGLQESHGRDGTRFCHLLDLPDKDYCFPNVLRNDYLNRHLGMVAQDPQGCLQLCLSEVANGLRNPVSMVHAGDGTHRFFVAEQVGVVWVYLPDGSRLEQPFLDLKNIVLTTPWIGDERGFLGLAFHPKFRHNRKFYIYYSCLDKKKVEKIRISEMKVSRADPNKADLKSERVILEIEEPASNHNGGQLLFGLDGYMYIFTGDGGQAGDPFGLFGNAQNKSSLLGKVLRIDVNRAGSHGKRYRVPSDNPFVSEPGAHPAIYAYGIRNMWRCAVDRGDPITRQGRGRIFCGDVGQNRFEEVDLILKGGNYGWRAKEGFACYDKKLCHNASLDDVLPIYAYGHAVGKSVTGGYVYRGCESPNLNGLYIFGDFMSGRLMALQEDRKNKKWKKQDLCLGSTTSCAFPGLISTHSKFIISFAEDEAGELYFLATSYPSAYAPRGSIYKFVDPSRRAPPGKCKYKPVPVRTKSKRIPFRPLAKTVLDLLKEQSEKAARKSSSATLASGPAQGLSEKGSSKKLASPTSSKNTLRGPGTKKKARVGPHVRQGKRRKSLKSHSGRMRPSAEQKRAGRSLP

SEQ ID NO: 83 - DNA, 1342 bp, Homo sapiens
catggagcctcttgcagcttacccgctaaaatgttccgggcccagagcaaaggtatttgcagttttgctgtctatagttctatgcacagtaacgctatttcttctacaactaaaattcctcaaacctaaaatcaacagcttttatgcctttgaagtgaaggatgcaaaaggaagaactgtttctctggaaaagtataaaggcaaagtttcactagttgtaaacgtggccagtgactgccaactcacagacagaaattacttagggctgaaggaactgcacaaagagtttggaccatcccacttcagcgtgttggcttttccctgcaatcagtttggagaatcggagccccgcccaagcaaggaagtagaatcttttgcaagaaaaaactacggagtaactttccccatcttccacaagattaagattctaggatctgaaggagaacctgcatttagatttcttgttgattcttcaaagaaggaaccaaggtggaatttttggaagtatcttgtcaaccctgagggtcaagttgtgaagttctggaggccagaggagcccattgaagtcatcaggcctgacatagcagctctggttagacaagtgatcataaaaaagaaagaggatctatgagaatgccattgcgtttctaatagaacagagaaatgtctccatgagggtttggtctcattttaaacattttttttttggagacagtgtctcactctgtcacccaggctggagtgcagtagtgcgttctcagctcattgcaacctctgcctttttaaacatgctattaaatgtggcaatgaaggatttttttttaatgttatcttgctattaagtggtaatgaatgttcccaggatgaggatgttacccaaagcaaaaatcaagagtagccaaagaatcaacatgaaatatattaactacttcctctgaccatactaaagaattcagaatacacagtgaccaatgtgcctcaatatcttattgttcaacttgacattttctaggactgtacttgatgaaaatgccaacacactagaccactctttggattcaagagcactgtgtatgactgaaatttctggaataactgtaaatggttatgttaatggaataaaacacaaatgttgaaaaatgtaaaatatatatacatagattcaaatccttatatatgtatgcttgttttgtgtacaggattttgttttttctttttaagtacaggttcctagtgttttactataactgtcactatgtatgtaactgacatatataaatagtcatttataaatgaccgtattataacatttgaaaaagtcttcatcaaaaaaaaaaaaaaa

SEQ ID NO: 84 - protein, 209 aa, Homo sapiens
MEPLAAYPLKCSGPRAKVFAVLLSIVLCTVTLFLLQLKFLKPKINSFYAFEVKDAKGRTVSLEKYKGKVSLVVNVASDCQLTDRNYLGLKELHKEFGPSHFSVLAFPCNQFGESEPRPSKEVESFARKNYGVTFPIFHKIKILGSEGEPAFRFLVDSSKKEPRWNFWKYLVNPEGQVVKFWRPEEPIEVIRPDIAALVRQVIIKKKEDL

SEQ ID NO: 85 - DNA, 660 bp, Homo sapiens
cggccagggcgccgacagcccgacctcaccaggagaacatgcagctcggcactgggctcctgctggccgccgtcctgagcctgcagctggctgcagccgaagccatatggtgtcaccagtgcacgggcttcggagggtgctcccatggatccagatgcctgagggactccacccactgtgtcaccactgccacccgggtcctcagcaacaccgaggatttgcctctggtcaccaagatgtgccacataggctgccccgatatccccagcctgggcctgggcccctacgtatccatcgcttgctgccagaccagcctctgcaaccatgactgacggctgccctcctccaggcccccggacgctcagcccccacagcccccacagcctggcgccagggctcacggccgcccctccctcgagactggccagcccacctctcccggcctctgcagccaccgtccagcaccgcttgtcctagggaagtcctgcgtggagtcttgcctcaatctgctgccgtccaagcctggggcccatcgtgcctgccgccccttcaggtcccgacctccccacaataaaatgtgattggatcgtgtggtacaaaaaaaaaaaaaaaaaaaaaaaaaaaaaaaaaaaaaaaaaaaaaaaaaaaaaaaaaaaaaaa

SEQ ID NO: 86 - protein, 97 aa, Homo sapiens
MQLGTGLLLAAVLSLQLAAAEAIWCHQCTGFGGCSHGSRCLRDSTHCVTTATRVLSNTEDLPLVTKMCHIGCPDIPSLGLGPYVSIACCQTSLCNHD

SEQ ID NO: 87 - DNA, 3121 bp, Homo sapiens
gcagtgctgctgaccatgagccaccaggtaaagggcctgaaagaggaaggtgacaggctgggaaggagatcctcaagcaagcgggctctcaaagccgaggggaccccaggcaggcgcggagctcagcgaagccagaaggagcgcgccgggggcagcccaagcccggggtctccccggaggaagcaaacagggcgcaggagacacagagaagagctgggggagcaggagcggggcgaggcagagaggacctgcgagggcaggagaaagcgcgacgagagggcctccttccaggagcggacagcagccccaaagagggaaaaggagattccgaggaaggaggagaagtcgaagcggcagaagaaacccaggtcatcctccttggcctccagtgcctctggtggggagtccctgtccgaggaggaactggcccagatcctggagcaggtggaagaaaaaaagaagctcattgccaccatgcggagcaagccctggcccatggcgaagaagctgacagagctcagggaggcccaggaatttgtggagaagtatgaaggtgccttgggaaaggggaaaggcaagcaactatatgcctacaagatgctgatggccaagaaatgggtcaaatttaagagagactttgataatttcaagactcaatgtatcccctgggaaatgaagatcaaggacattgaaagtcactttggttcttcagtggcatcgtatttcatctttctccgatggatgtatggagttaaccttgtcctttttggcttaatatttggtctagtcataatcccagaggtactgatgggcatgccctatgggagtattcccagaaagacagtgcctcgggctgaggaagaaaaggccatggatttttctgtcctttgggattttgagggctatatcaagtactctgcactcttctatggctactacaacaaccagaggaccatcgggtggctgaggtaccggctgcctatggcttactttatggtgggggtcagcgtgttcggctacagcctgattattgtcattcgatcgatggccagcaatacccaaggaagcacaggcgaaggggagagtgacaacttcacattcagcttcaagatgttcaccagctgggactacctgatcgggaattcagagacagctgataacaaatatgcatccatcaccaccagcttcaaggaatcaatagtggatgaacaagagagtaacaaagaagaaaatatccatctgacaagatttcttcgtgtcctggccaactttctcatcatctgctgtttgtgtggaagtgggtacctcatttactttgtggttaagcgatctcagcaattctccaaaatgcagaatgtcagctggtatgaaaggaatgaggtagagatcgtgatgtccctgcttggaatgttttgtccccctctgtttgaaaccatcgctgccctggagaattaccacccacgcactggactgaagtggcagctgggacgcatctttgcactcttcctggggaacctctacacatttctcttggccctgatggatgacgtccacctcaagcttgctaatgaagagacaataaagaacatcactcactggactctgtttaactattacaactcttctggttggaacgagagtgtcccccgaccacccctgcaccctgcagatgtgccccggggttcttgctgggagacagctgtgggcattgaattcatgaggctgacggtgtctgacatgctggtaacgtacatcaccatcctgctgggggacttcctacgggcttgttttgtgcggttcatgaactactgctggtgctgggacttggaggctggatttccttcatatgctgagtttgatattagtggaaatgtgctgggtttgatcttcaaccaaggaatgatctggatgggctccttctatgctccaggcctggtgggcattaatgtgctgcgcctgctgacctccatgtacttccagtgctgggcggtgatgagcagcaacgtaccccatgaacgcgtgttcaaagcctcccgatccaacaacttctacatgggcctcctgctgctggtgctcttcctcagcctcctgccggtggcctacaccatcatgtccctcccaccctcctttgactgcgggccgttcagtgggaaaaacagaatgtacgatgtcctccaagagaccattgaaaacgatttcccaaccttcctgggcaagatctttgctttcctcgccaatccaggcctgatcatcccagccatcctgctgatgttcttggccatttactacctgaactcagtttccaaaagcctttcccgagctaatgcccagctgaggaagaaaatccaagtgctccgtgaagttgagaagagtcacaaatctgtaaaaggcaaagccacagccagagattcagaggacacacctaaaagcagctccaaaaatgccacccagctccaactcaccaaggaagagaccactcctccctctgccagccaaagccaggccatggacaagaaggcgcagggccctgggacctccaattctgccagcaggaccacactgcctgcctctggacaccttcctatatctcggccccctggaatcggaccagattctggccacgccccatctcagactcatccgtggaggtcagcctctggaaagagtgctcagagacctccccactgacggctaggactccagggagcctcgaccctagggctgatcctcaagtaccccagtttcacacataccaaaccaaggttctctcccctctttcctctcacatacatgctctgtctcctctcttggaatgcatgaactttgattccttcaggcccttgtcagctaccgaaggaggaagacagtggcttcacctgtcctttagggaagctggagccatctctgcactaactgccctcccaaatatcttggttcagacagctctgaaccccacgctcacagtggtcgaccttgcctcccgattttcggagttggggaagggccatgaccaccctcgtagactttttccatgggatacagtttaggacacgggtttctgccagcttccctaaccaggagggggatggagaagggcctacatttctcaatccagaggaag

SEQ ID NO: 88 - protein, 890 aa, Homo sapiens
MSHQVKGLKEEGDRLGRRSSSKRALKAEGTPGRRGAQRSQKERAGGSPSPGSPRRKQTGRRRHREELGEQERGEAERTCEGRRKRDERASFQERTAAPKREKEIPRKEEKSKRQKKPRSSSLASSASGGESLSEEELAQILEQVEEKKKLIATMRSKPWPMAKKLTELREAQEFVEKYEGALGKGKGKQLYAYKMLMAKKWVKFKRDFDNFKTQCIPWEMKIKDIESHFGSSVASYFIFLRWMYGVNLVLFGLIFGLVIIPEVLMGMPYGSIPRKTVPRAEEEKAMDFSVLWDFEGYIKYSALFYGYYNNQRTIGWLRYRLPMAYFMVGVSVFGYSLIIVIRSMASNTQGSTGEGESDNFTFSFKMFTSWDYLIGNSETADNKYASITTSFKESIVDEQESNKEENIHLTRFLRVLANFLIICCLCGSGYLIYFVVKRSQQFSKMQNVSWYERNEVEIVMSLLGMFCPPLFETIAALENYHPRTGLKWQLGRIFALFLGNLYTFLLALMDDVHLKLANEETIKNITHWTLFNYYNSSGWNESVPRPPLHPADVPRGSCWETAVGIEFMRLTVSDMLVTYITILLGDFLRACFVRFMNYCWCWDLEAGFPSYAEFDISGNVLGLIFNQGMIWMGSFYAPGLVGINVLRLLTSMYFQCWAVMSSNVPHERVFKASRSNNFYMGLLLLVLFLSLLPVAYTIMSLPPSFDCGPFSGKNRMYDVLQETIENDFPTFLGKIFAFLANPGLIIPAILLMFLAIYYLNSVSKSLSRANAQLRKKIQVLREVEKSHKSVKGKATARDSEDTPKSSSKNATQLQLTKEETTPPSASQSQAMDKKAQGPGTSNSASRTTLPASGHLPISRPPGIGPDSGHAPSQTHPWRSASGKSAQRPPH

SEQ ID NO: 89 - DNA, 3437 bp, Homo sapiens
caggaccaggtcttcctacgctggagcagcggggagacagccaccatgcacatcctcgtggtccatgccatggtgatcctgctgacgctgggcccgcctcgagccgacgacagcgagttccaggcgctgctggacatctggtttccggaggagaagccactgcccaccgccttcctggtggacacatcggaggaggcgctgctgcttcctgactggctgaagctgcgcatgatccgttctgaggtgctccgcctggtggacgccgccctgcaggacctggagccgcagcagctgctgctgttcgtgcagtcgtttggcatccccgtgtccagcatgagcaaactcctccagttcctggaccaggcagtggcccacgacccccagactctggagcagaacatcatggacaagaattacatggcccacctggtggaggtccagcatgagcgcggcgcctccggaggccagactttccactccttgctcacagcctccctgccgccccgccgagacagcacagaggcacccaaaccaaagagcagcccagagcagcccataggccagggccggattcgggtggggacccagctccgggtgctgggccctgaggacgacctggctggcatgttcctccagattttcccgctcagcccggaccctcggtggcagagctccagtccccgccccgtggccctcgccctgcagcaggccctgggccaggagctggcccgcgtcgtccagggcagccccgaggtgccgggcatcacggtgcgtgtcctgcaggccctcgccaccctgctcagctccccacacggcggtgccctggtgatgtccatgcaccgtagccacttcctggcctgcccgctgctgcgccagctctgccagtaccagcgctgtgtgccacaggacaccggcttctcctcgctcttcctgaaggtgctcctgcagatgctgcagtggctggacagccctggcgtggagggcgggcccctgcgggcacagctcaggatgcttgccagccaggcctcagccgggcgcaggctcagtgatgtgcgaggggggctcctgcgcctggccgaggccctggccttccgtcaggacctggaggtggtcagctccaccgtccgtgccgtcatcgccaccctgaggtctggggagcagtgcagcgtggagccggacctgatcagcaaagtcctccaggggctgatcgaggtgaggtccccccacctggaggagctgctgactgcattcttctctgccactgcggatgctgcctccccgtttccagcctgtaagcccgttgtggtggtgagctccctgctgctgcaggaggaggagcccctggctggggggaagccgggtgcggacggtggcagcctggaggccgtgcggctggggccctcgtcaggcctcctagtggactggctggaaatgctggaccccgaggtggtcagcagctgccccgacctgcagctcaggctgctcttctcccggaggaagggcaaaggtcaggcccaggtgccctcgttccgtccctacctcctgaccctcttcacgcatcagtccagctggcccacactgcaccagtgcatccgagtcctgctgggcaagagccgggaacagaggttcgacccctctgcctctctggacttcctctgggcctgcatccatgttcctcgcatctggcaggggcgggaccagcgcaccccgcagaagcggcgggaggagctggtgctgcgggtccagggcccggagctcatcagcctggtggagctgatcctggccgaggcggagacgcggagccaggacggggacacagccgcctgcagcctcatccaggcccggctgcccctgctgctcagctgctgctgtggggacgatgagagtgtcaggaaggtgacggagcacctgtcaggctgcatccagcagtggggagacagcgtgctgggaaggcgctgccgagaccttctcctgcagctctacctacagcggccggagctgcgggtgcccgtgcctgaggtcctactgcacagcgaaggggctgccagcagcagcgtctgcaagctggacggactcatccaccgcttcatcacgctccttgcggacaccagcgactcccgggcgttggagaaccgaggggcggatgccagcatggcctgccggaagctggcggtggcgcacccgctgctgctgctcaggcacctgcccatgatcgcggcgctcctgcacggccgcacccacctcaacttccaggagttccggcagcagaaccacctgagctgcttcctgcacgtgctgggcctgctggagctgctgcagccgcacgtgttccgcagcgagcaccagggggcgctgtgggactgccttctgtccttcatccgcctgctgctgaattacaggaagtcctcccgccatctggctgccttcatcaacaagtttgtgcagttcatccataagtacattacctacaatgccccagcagccatctccttcctgcagaagcacgccgacccgctccacgacctgtccttcgacaacagtgacctggtgatgctgaaatccctccttgcagggctcagcctgcccagcagggacgacaggaccgaccgaggcctggacgaagagggcgaggaggagagctcagccggctccttgcccctggtcagcgtctccctgttcacccctctgaccgcggccgagatggccccctacatgaaacggctttcccggggccaaacggtggaggatctgctggaggttctgagtgacatagacgagatgtcccggcggagacccgagatcctgagcttcttctcgaccaacctgcagcggctgatgagctcggccgaggagtgttgccgcaacctcgccttcagcctggccctgcgctccatgcagaacagccccagcattgcagccgctttcctgcccacgttcatgtactgcctgggcagccaggactttgaggtggtgcagacggccctccggaacctgcctgagtacgctctcctgtgccaagagcacgcggctgtgctgctccaccgggccttcctggtgggcatgtacggccagatggaccccagcgcgcagatctccgaggccctgaggatcctgcatatggaggccgtgatgtgagcctgtggcagccgacccccctccaagccccggcccgtcccgtccccggggatcctcgaggcaaagcccaggaagcgtgggcgttgctggtctgtccgaggaggtgagggcgccgagccctgaggccaggcaggcccaggagcaatactccgagccctggggtggctccgggccggccgctggcatcaggggccgtccagcaagccctcattcaccttctgggccacagccctgccgcggagcggcggatccccccgggcatggcctgggctggttttgaatgaaacgacctgaactgtcaa

SEQ ID NO: 90 - protein, 1029 aa, Homo sapiens
MHILVVHAMVILLTLGPPRADDSEFQALLDIWFPEEKPLPTAFLVDTSEEALLLPDWLKLRMIRSEVLRLVDAALQDLEPQQLLLFVQSFGIPVSSMSKLLQFLDQAVAHDPQTLEQNIMDKNYMAHLVEVQHERGASGGQTFHSLLTASLPPRRDSTEAPKPKSSPEQPIGQGRIRVGTQLRVLGPEDDLAGMFLQIFPLSPDPRWQSSSPRPVALALQQALGQELARVVQGSPEVPGITVRVLQALATLLSSPHGGALVMSMHRSHFLACPLLRQLCQYQRCVPQDTGFSSLFLKVLLQMLQWLDSPGVEGGPLRAQLRMLASQASAGRRLSDVRGGLLRLAEALAFRQDLEVVSSTVRAVIATLRSGEQCSVEPDLISKVLQGLIEVRSPHLEELLTAFFSATADAASPFPACKPVVVVSSLLLQEEEPLAGGKPGADGGSLEAVRLGPSSGLLVDWLEMLDPEVVSSCPDLQLRLLFSRRKGKGQAQVPSFRPYLLTLFTHQSSWPTLHQCIRVLLGKSREQRFDPSASLDFLWACIHVPRIWQGRDQRTPQKRREELVLRVQGPELISLVELILAEAETRSQDGDTAACSLIQARLPLLLSCCCGDDESVRKVTEHLSGCIQQWGDSVLGRRCRDLLLQLYLQRPELRVPVPEVLLHSEGAASSSVCKLDGLIHRFITLLADTSDSRALENRGADASMACRKLAVAHPLLLLRHLPMIAALLHGRTHLNFQEFRQQNHLSCFLHVLGLLELLQPHVFRSEHQGALWDCLLSFIRLLLNYRKSSRHLAAFINKFVQFIHKYITYNAPAAISFLQKHADPLHDLSFDNSDLVMLKSLLAGLSLPSRDDRTDRGLDEEGEEESSAGSLPLVSVSLFTPLTAAEMAPYMKRLSRGQTVEDLLEVLSDIDEMSRRRPEILSFFSTNLQRLMSSAEECCRNLAFSLALRSMQNSPSIAAAFLPTFMYCLGSQDFEVVQTALRNLPEYALLCQEHAAVLLHRAFLVGMYGQMDPSAQISEALRILHMEAVM

SEQ ID NO: 91 - DNA, 1728 bp, Homo sapiens
ggccggacgcctccgcgttacgggatgaattaacggcgggttccgcacggaggttgtgacccctacggagccccagcttgcccacgcaccccactcggcgtcgcgcggcgtgccctgcttgtcacaggtgggaggctggaactatcaggctgaaaaacagagtgggtactctcttctgggaagctggcaacaaatggatgatgtgatatatgcattccaggggaagggaaattgtggtgcttctgaacccatggtcaattaacgaggcagtttctagctactgcacgtacttcataaagcaggactctaaaagctttggaatcatggtgtcatggaaagggatttactttatactgactctgttttggggaagcttttttggaagcattttcatgctgagtccctttttacctttgatgtttgtaaacccatcttggtatcgctggatcaacaaccgccttgtggcaacatggctcaccctacctgtggcattattggagaccatgtttggtgtaaaagtgattataactggggatgcatttgttcctggagaaagaagtgtcattatcatgaaccatcggacaagaatggactggatgttcctgtggaattgcctgatgcgatatagctacctcagattggagaaaatttgcctcaaagcgagtctcaaaggtgttcctggatttggttgggccatgcaggctgctgcctatatcttcattcataggaaatggaaggatgacaagagccatttcgaagacatgattgattacttttgtgatattcacgaaccacttcaactcctcatattcccagaagggactgatctcacagaaaacagcaagtctcgaagtaatgcatttgctgaaaaaaatggacttcagaaatatgaatatgttttacatccaagaactacaggctttacttttgtggtagaccgtctaagagaaggtaagaaccttgatgctgtccatgatatcactgtggcgtatcctcacaacattcctcaatcagagaagcacctcctccaaggagactttcccagggaaatccactttcacgtccaccggtatccaatagacaccctccccacatccaaggaggaccttcaactctggtgccacaaacggtgggaagagaaagaagagaggctgcgttccttctatcaaggggagaagaatttttattttaccggacagagtgtcattccaccttgcaagtctgaactcagggtccttgtggtcaaattgctctctatactgtattggaccctgttcagccctgcaatgtgcctactcatatatttgtacagtcttgttaagtggtattttataatcaccattgtaatctttgtgctgcaagagagaatatttggtggactggagatcatagaacttgcatgttaccgacttttacacaaacagccacatttaaattcaaagaaaaatgagtaagattataaggtttgccatgtgaaaacctagagcatattttggaaatgttctaaacctttctaagctcagatgcatttttgcatgactatgtcgaatatttcttactgccatcattatttgttaaagatattttgcacttaattttgtgggaaaaatattgctacaattttttttaatctctgaatgtaatttcgatactgtgtacatagcagggagtgatcggggtgaaataacttgggccagaatattattaaacaatcatcaggcttttaaa

SEQ ID NO: 92 - protein, 414 aa, Homo sapiens [sequence omitted]

SEQ ID NO: 93 - DNA, 2164 bp, Homo sapiens
caccggagggcacgcagctgacggagctgcgctgcgttcgcctcgtttgcctcgcgccctccactggagctgttcgcgcctcccggctcccaccgcagcccacccggcagaggagtcgctaccagcgcccagtgcgctctgtcagtccgcaaactccttgccgcccgccccgggctgggcaccaaataccaggctaccatggtctacaagactctcttcgctctttgcatcttaactgcaggatggagggtacagagtctgcctacatcagctcctttgtctgtttctcttccgacaaacattgtaccaccgaccaccatctggactagctctccacaaaacactgatgcagacactgcctccccatccaacggcactcacaacaactcggtgctcccagttacagcatcagccccaacatctctgcttcctaagaacatttccatagagtccagagaagaggagatcaccagcccaggttcgaattgggaaggcacaaacacagacccctcaccttctgggttctcgtcaacaagcggtggagtccacttaacaaccacgttggaggaacacagctcgggcactcctgaagcaggcgtggcagctacactgtcgcagtccgctgctgagcctcccacactcatctcccctcaagctccagcctcatcaccctcatccctatcaacctcaccacctgaggtcttttctgcctccgttactaccaaccatagctccactgtgaccagcacccaacccactggagctccaactgcaccagagtccccgacagaggagtccagctctgaccacacacccacttcacatgccacagctgagccagtgccccaggagaaaacacccccaacaactgtgtcaggcaaagtgatgtgtgagctcatagacatggagaccaccaccacctttcccagggtgatcatgcaggaagtagaacatgcattaagttcaggcagcatcgccgccattaccgtgacagtcattgccgtggtgctgctggtgtttggagttgcagcctacctaaaaatcaggcattcctcctatggaagacttttggacgaccatgactacgggtcctggggaaactacaacaaccctctgtacgatgactcctaacaatggaatatggcctgggatgaggattaactgttctttatttataagtgcttatccagtagaattaataagtacctgatgcgcattgaacgacaatcttaagccctgttttgttggtatggttgtttttgttttcctccctctcctctggctgctacaacttcccctttctggtacaagaagaaccattctttaaaggtgagtggaggctgatttgcagctgaagtgggccagccttgcaccagccaggccagaccaccatggtgaaggcttctttccccactgcaggacccactttgagaaggatcgaggaggaggatttgggttgttttgttaggggttactttcaggggaacatttcatttgtgttatttcttaaacttctatttaggaaattacattaagtattaatgaggggaaaggaaatgagctctacgaggatttcaccttgcatgggagagagcagggttttctcagattcctttttaatctctatttatctggttgtttctgacaggatgctgcctgcttggctctacgagctggaaagcagcttcttagctgcctaattaatgaaagatgaaaataggaagtgccctggagggggccagcaggtcacggggcagaatctctcaggttgctgtgggatctcagtgtgcccctacctgttctcccctccaggccacctgtctctgtaaaggatgtctgctctgttcaaaaggcagctgggatcccagcccacaagtgatcagcagagttgcatttccaaagaaaaaggctatgagatgagctgagttatagagagaaagggagaggcatgtacggtgtggggaagtggaagagaagctggcgggggagaaggaggctaacctgcactgagtacttcattaggacaagtgagaatcagctattgataatggccagagatatccacagcttggaggagcccagagactgtttgctttatacccacacagcaactggtccactgctttactgtctgttggataatggctgtaaaatgtttaaaaac

SEQ ID NO: 94 - protein, 310 aa, Homo sapiens
MVYKTLFALCILTAGWRVQSLPTSAPLSVSLPTNIVPPTTIWTSSPQNTDADTASPSNGTHNNSVLPVTASAPTSLLPKNISIESREEEITSPGSNWEGTNTDPSPSGFSSTSGGVHLTTTLEEHSSGTPEAGVAATLSQSAAEPPTLISPQAPASSPSSLSTSPPEVFSASVTTNHSSTVTSTQPTGAPTAPESPTEESSSDHTPTSHATAEPVPQEKTPPTTVSGKVMCELIDMETTTTFPRVIMQEVEHALSSGSIAAITVTVIAVVLLVFGVAAYLKIRHSSYGRLLDDHDYGSWGNYNNPLYDDS

SEQ ID NO: 95 - DNA, 1941 bp, Homo sapiens
cagaatcgcagattgccagcccttttcccgacccctacggaaagacgagtccaggggccgtcctggcgaggtcaaaacatttagtctggtcttttcagcgtggaccctgccagcagccaggccatggagctctctgatgtcaccctcattgagggtgtgggtaatgaggtgatggtggtggcaggtgtggtggtgctgattctagccttggtcctagcttggctctctacctacgtagcagacagcggtagcaaccagctcctgggcgctattgtgtcagcaggcgacacatccgtcctccacctggggcatgtggaccacctggtggcaggccaaggcaaccccgagccaactgaactcccccatccatcagagggtaatgatgagaaggctgaagaggcgggtgaaggtcggggagactccactggggaggctggagctgggggtggtgttgagcccagccttgagcatctccttgacatccaaggcctgcccaaaagacaagcaggtgcaggcagcagcagtccagaggcccccctgagatctgaggatagcacctgcctccctcccagccctggcctcatcactgtgcggctcaaattcctcaatgataccgaggagctggctgtggctaggccagaggataccgtgggtgccctgaagagcaaatacttccctggacaagaaagccagatgaaactgatctaccagggccgcctgctacaagacccagcccgcacactgcgttctctgaacattaccgacaactgtgtgattcactgccaccgctcacccccagggtcagctgttccaggcccctcagcctccttggccccctcggccactgagccacccagccttggtgtcaatgtgggcagcctcatggtgcctgtctttgtggtgctgttgggtgtggtctggtacttccgaatcaattaccgccaattcttcacagcacctgccactgtctccctggtgggagtcaccgtcttcttcagcttcctagtatttgggatgtatggacgataaggacataggaagaaaatgaaaggcatggtctttctcctttatggcctccccacttttcctggccagagctgggcccaagggccggggagggaggggtggaaaggatgtgatggaaatctcctccataggacacaggaggcaagtatgcggcctccccttctcatccacaggagtacagatgtccctcccgtgcgagcacaactcaggtagaaatgaggatgtcatcttccttcacttttagggtcctctgaaggagttcaaagctgctggccaagctcagtggggagcctgggctctgagattccctcccacctgtggttctgactcttcccagtgtcctgcatgtctgcccccagcacccagggctgcctgcaagggcagctcagcatggccccagcacaactccgtagggagcctggagtatccttccatttctcagccaaatactcatcttttgagactgaaatcacactggcgggaatgaagattgtgccagccttctcttatgggcacctagccgccttcaccttcttcctctaccccttagcaggaatagggtgtcctcccttctttcaaagcactttgcttgcattttattttatttttttaagagtccttcatagagctcagtcaggaaggggatggggcaccaagccaagcccccagcattgggagcggccaggccacagctgctgctcccgtagtcctcaggctgtaagcaagagacagcactggcccttggccagcgtcctaccctgcccaactccaaggactgggtatggatcgctgggccctaggctcttgcttctggggctattggagggtcagtgtctgtgactgaataaagttccattttgtggaaaaaaaaaaaaaaaaaaaaaaaaaaaaaaaaaaaaaaaaaaaaaaaaaaaa

SEQ ID NO: 96 - protein, 301 aa, Homo sapiens
MELSDVTLIEGVGNEVMVVAGVVVLILALVLAWLSTYVADSGSNQLLGAIVSAGDTSVLHLGHVDHLVAGQGNPEPTELPHPSEGNDEKAEEAGEGRGDSTGEAGAGGGVEPSLEHLLDIQGLPKRQAGAGSSSPEAPLRSEDSTCLPPSPGLITVRLKFLNDTEELAVARPEDTVGALKSKYFPGQESQMKLIYQGRLLQDPARTLRSLNITDNCVIHCHRSPPGSAVPGPSASLAPSATEPPSLGVNVGSLMVPVFVVLLGVVWYFRINYRQFFTAPATVSLVGVTVFFSFLVFGMYGR

SEQ ID NO: 97 - DNA, 1664 bp, Homo sapiens
caaagccctaccctcaccattcaccaggtcctgtgggaagagcagcgtggaggtgggctgaggttagaaggtgcagagcgtggaagaagattgtgagctgagtattggacatctgttcttgaatagtccctgggcctgccataggaaaggaagttctccagggttacagttcttatccgcgtgaatacacatggctctgttacgaaaaattaatcaggtgctgctgttccttctgatcgtgaccctctgtgtgattctgtataagaaagttcataaggggactgtgcccaagaatgacgcagatgatgaatccgagactcctgaagaactggaagaagagattcctgtggtgatttgtgctgcagcagggaggatgggtgccactatggctgccatcaatagcatctacagcaacactgacgccaacatcttgttctatgtagtgggactccggaatactctgactcgaatacgaaaatggattgaacattccaaactgagagaaataaactttaaaatcgtggaattcaacccgatggtcctcaaagggaagatcagaccagactcatcgaggcctgaattgctccagcctctgaactttgttcgattttatctccctctacttatccaccaacacgagaaagtcatctatttggacgatgatgtaattgtacaaggtgatatccaagaactgtatgacaccaccttggccctgggccacgcggcggctttctcagatgactgcgatttgccctctgctcaggacataaacagactcgtgggacttcagaacacatatatgggctatctggactaccggaagaaggccatcaaggaccttggcatcagccccagcacctgctctttcaatcctggtgtgattgttgccaacatgacagaatggaagcaccagcgcatcaccaagcaattggagaaatggatgcaaaagaatgtggaggaaaacctctatagcagctccctgggaggaggggtggccacctccccaatgctgattgtgtttcatgggaaatattccacaattaaccccctgtggcacataaggcacctgggctggaatccagatgccagatattcggagcattttctgcaggaagctaaattactccactggaatggaagacataaaccttgggacttccctagtgttcacaacgacttatgggaaagctggtttgttcctgaccctgcagggatatttaaactcaatcaccatagctgatataactctacccttaaaatattccctgtatagaaatgtggaattgtccctttgtagccaactataacattgttctttatgaatattacctttgatacatatgatccacaatataaaaaccaaaaactactgtgtgcaaattataccttggaccatataggcattgattaacttctttaagtacatgtgataactatggaaatcaagattatgtgactgaaaaacataaaggaagagacccatctagataacagcaatcaacctgcttaattctgaatgacaattatatccacaaatttttaaaacttctacatgtatttttcacatgaagatctccttaacaggttgccaaccttttcttttataaaactattacatttaaaatatggacgtctgaaaaataaaatattcatcatttttaaaa

SEQ ID NO: 98 - protein, 349 aa, Homo sapiens
MALLRKINQVLLFLLIVTLCVILYKKVHKGTVPKNDADDESETPEELEEEIPVVICAAAGRMGATMAAINSIYSNTDANILFYVVGLRNTLTRIRKWIEHSKLREINFKIVEFNPMVLKGKIRPDSSRPELLQPLNFVRFYLPLLIHQHEKVIYLDDDVIVQGDIQELYDTTLALGHAAAFSDDCDLPSAQDINRLVGLQNTYMGYLDYRKKAIKDLGISPSTCSFNPGVIVANMTEWKHQRITKQLEKWMQKNVEENLYSSSLGGGVATSPMLIVFHGKYSTINPLWHIRHLGWNPDARYSEHFLQEAKLLHWNGRHKPWDFPSVHNDLWESWFVPDPAGIFKLNHHS

SEQ ID NO: 99 - DNA, 1904 bp, Homo sapiens
gcgccggctccgcgcctcgcgcccagtccgcgggccgcgccgccgctcccgccgctcccgccgctcccgcagccgccccgccgcccgcccggagccccgcgtccctaggcctggctcccgcctgcccgagacccgcccagcctgccccgctcagccgccagagaagatgcggctgctcccggaatggttcctcttgctctttggcccgtggctccttaggaaggccgtcagtgcccagataccagagtccggaaggccgcagtacctggggctgcgccccgccgcggccggagcgggtgcccccggccagcagctcccagagccaaggtcttcggacggcctaggcgtgggccgcgcctggagctgggcctggccgaccaaccacacgggggcgctggcccgggcaggggcagccggggcgttgcccgcgcagcgcaccaagaggaagccgtccatcaaggcggcgcgcgccaaaaagatcttcggctggggggacttctactttcgggtgcataccctcaagttttcgctgctggtgaccggcaagatcgtggaccatgtgaacggtaccttcagtgtgtatttccgccacaactcgtccagcctgggcaacctcagtgtcagcatcgtgccgccctccaagcgtgtcgagttcggaggagtctggctgcccgggcctgtcccccaccctctgcagtctacgctcgccctggagggggtgcttcctgggctggggcccccgctggggatggcagcagcagcggcggggccggggcttgggggctccctcgggggcgcactggcggggccgcttgggggcgcgttgggagtgcctggggccaaagagtcacgcgctttcaattgccacgtggagtatgagaagacaaaccgcgcgcgcaagcaccgaccgtgcctgtacgacccgtcgcaggtgtgtttcaccgagcacacgcagagccaggccgcctggctctgtgccaagcccttcaaagtcatctgtatcttcgtctctttcctcagctttgactacaaactggtgcagaaggtgtgcccagactataacttccagagtgagcacccctacttcggatagcgcccctccccagccagtcctgagcctcccgccaaatcccagcctcactaggtgggacccccttcccagtgttctgccgctcctgtggccatgtcgcccactccttccactctgggggcggaggggaatggcttctcgggaccctcagctagcgtgggtgcccttttccttatgcggagtgcccgcaaggctggggtagccccctccagtacaccccaaagtgaaagggataagagtgcagccccagaataggcggggcttggaggcggtcccaatgtcccctgggtccacagtgggtccccttttcacccttggcgctaggctgcgcactccctttccccgcagctttaataactcctggcctggcaccctcaccccaccctgactttcccatcccccagcgcttgtcctgcttcaccataccccgcctaagactgtaaaggcctaaaaacctcggcctgtcctcccaccattctgcctgccatatgcctgtccccttttcctccaaaccctattagggtaccggaagcagaacccctgggctgaggccctggccctgcccccggcccctgcccctgcccgcccccctccagtccaggcagtcgagctccacctgccctctcctgctgcttcctctcggtgatattttttctacgccaaaacagacgggaaagggaacaaaataaagtgaaatcccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 100 - protein, 308 aa, Homo sapiens
MRLLPEWFLLLFGPWLLRKAVSAQIPESGRPQYLGLRPAAAGAGAPGQQLPEPRSSDGLGVGRAWSWAWPTNHTGALARAGAAGALPAQRTKRKPSIKAARAKKIFGWGDFYFRVHTLKFSLLVTGKIVDHVNGTFSVYFRHNSSSLGNLSVSIVPPSKRVEFGGVWLPGPVPHPLQSTLALEGVLPGLGPPLGMAAAAAGPGLGGSLGGALAGPLGGALGVPGAKESRAFNCHVEYEKTNRARKHRPCLYDPSQVCFTEHTQSQAAWLCAKPFKVICIFVSFLSFDYKLVQKVCPDYNFQSEHPYFG

SEQ ID NO: 101 - DNA, 2837 bp, Homo sapiens
gggaagggatgcaaggaagccctccggcgctgcgctccgaggcgggagacagcgtcccgctgaaaatgtgtgtctgacatgcaagctcagtggggcagagacccgtggattgctgtgccctgccctccggacctggatcatgaaggtgttgggaagaagcttcttctgggtgctgtttcccgtccttccctgggcggtgcaggctgtggagcacgaggaggtggcgcagcgtgtgatcaaactgcaccgcgggcgaggggtggctgccatgcagagccggcagtgggtccgggacagctgcaggaagctctcagggcttctccgccagaagaatgcagttctgaacaaactgaaaactgcaattggagcagtggagaaagacgtgggcctgtcggatgaagagaaactgtttcaggtgcacacgtttgaaattttccagaaagagctgaatgaaagtgaaaattccgttttccaagctgtctacggactgcagagagccctgcagggggattacaaagatgtcgtgaacatgaaggagagcagccggcagcgcctggaggccctgagagaggctgcaataaaggaagaaacagaatatatggaacttctggcagcagaaaaacatcaagttgaagcccttaaaaatatgcaacatcaaaaccaaagtttatccatgcttgacgagattcttgaagatgtaagaaaggcagcggatcgtctggaggaagagatagaggaacatgcttttgacgacaataaatcagtcaagggggtcaattttgaggcagttctgagggtggaggaagaagaggccaattctaagcaaaatataacaaaacgagaagtggaggatgacttgggtcttagcatgctgattgactcccagaacaaccagtatattttgaccaagcccagagattcaaccatcccacgtgcagatcaccactttataaaggacattgttaccataggaatgctgtccttgccttgtggctggctatgtacagccataggattgcctacaatgtttggttatattatttgtggtgtacttctgggaccttcaggactaaatagtattaagtctattgtgcaagtggagacattaggagaatttggggtgttttttactctttttcttgttggcttagaattttctccagaaaagctaagaaaggtgtggaagatttccttacaagggccgtgttacatgacactgttaatgattgcatttggcttgctgtgggggcatctcttgcggatcaaacccacgcagagcgtcttcatttccacgtgtctgtccttgtcaagcacacccctcgtgtccaggttcctcatgggcagtgctcggggtgacaaagaaggcgacattgactacagcaccgtgctcctcggcatgctggtgacgcaggacgtgcagctcgggctcttcatggccgtcatgccgactctcatacaggcgggcgccagtgcatcttctagcattgtcgtggaagttctccgaatcctggttttgattggtcagattcttttttcactagcggcggtttttcttttatgtcttgttataaagaagtatctcattggaccctattatcggaagctgcacatggaaagcaaggggaacaaagaaatcctgatcttgggaatatctgcctttatcttcttaatgttaacggtcacggagctgctggacgtctccatggagctgggctgtttcctggctggagcgctcgtctcctctcagggccccgtggtcaccgaggagatcgccacctccatcgaacccatccgcgacttcctggccatcgttttcttcgcctccatagggctccacgtgttccccacgtttgtggcgtacgagctcacggtgctggtgttcctcaccttgtcagtggtggtgatgaagtttctcctggcggcgctggtcctgtctctcattctgccgaggagcagccagtacatcaagtggatcgtctctgcggggcttgcccaggtcagcgagttttcctttgtcctggggagccgggcgcgaagagcgggcgtcatctctcgggaggtgtacctccttatactgagtgtgaccacgctcagcctcttgctcgccccggtgctgtggagagctgcaatcacgaggtgtgtgcccagaccggagagacggtccagcctctgatggctcggagatgatggaccgtggaagggaagcgtctgtggggagtgagcgcttagatggccagcagctgctccttctgggaagctcgcaccttggcaacagaacagccctctagcagagcgtcagtgcagtcgtgttatcccggcttttacagaatattcttgtcctattttagaattttccggagtagtttatttgcagtctgttgattatgtgcagtagacccgggacactgcgttttaccgatcaccttgaatgtggtgcctggatgtgccttttttttttttccctgaaattattattaattttctattgtgagttcatcagttcatagtttttttagtaaagaagcaaaattaaaaggcttttaaaaatgtacaacttcagaattataatctgttagtcaaatatttgttattaaacatttctgtaatatgaagttgtaatcctggccgtgagcttggaagcttacttttgattcttaaagcctatgttttctaaaatgagacaaatacggatgtctatttgccttttattgtaacttttaaatgaaataatttcatgtcaatttctattagatatatcacttaaaatatttggttttaaatcacaagaatatgtattctttaataaagataatttatgatcatggtaaaaaaaaaaa

SEQ ID NO: 102 - protein, 677 aa, Homo sapiens
MKVLGRSFFWVLFPVLPWAVQAVEHEEVAQRVIKLHRGRGVAAMQSRQWVRDSCRKLSGLLRQKNAVLNKLKTAIGAVEKDVGLSDEEKLFQVHTFEIFQKELNESENSVFQAVYGLQRALQGDYKDVVNMKESSRQRLEALREAAIKEETEYMELLAAEKHQVEALKNMQHQNQSLSMLDEILEDVRKAADRLEEEIEEHAFDDNKSVKGVNFEAVLRVEEEEANSKQNITKREVEDDLGLSMLIDSQNNQYILTKPRDSTIPRADHHFIKDIVTIGMLSLPCGWLCTAIGLPTMFGYIICGVLLGPSGLNSIKSIVQVETLGEFGVFFTLFLVGLEFSPEKLRKVWKISLQGPCYMTLLMIAFGLLWGHLLRIKPTQSVFISTCLSLSSTPLVSRFLMGSARGDKEGDIDYSTVLLGMLVTQDVQLGLFMAVMPTLIQAGASASSSIVVEVLRILVLIGQILFSLAAVFLLCLVIKKYLIGPYYRKLHMESKGNKEILILGISAFIFLMLTVTELLDVSMELGCFLAGALVSSQGPVVTEEIATSIEPIRDFLAIVFFASIGLHVFPTFVAYELTVLVFLTLSVVVMKFLLAALVLSLILPRSSQYIKWIVSAGLAQVSEFSFVLGSRARRAGVISREVYLLILSVTTLSLLLAPVLWRAAITRCVPRPERRSSL

SEQ ID NO: 103 - DNA, 2362 bp, Homo sapiens
gacggaagaacagcgctcccgaggccgcgggagcctgcagagaggacagccggcctgcgccgggacatgcggccccaggagctccccaggctcgcgttcccgttgctgctgttgctgttgctgctgctgccgccgccgccgtgccctgcccacagcgccacgcgcttcgaccccacctgggagtccctggacgcccgccagctgcccgcgtggtttgaccaggccaagttcggcatcttcatccactggggagtgttttccgtgcccagcttcggtagcgagtggttctggtggtattggcaaaaggaaaagataccgaagtatgtggaatttatgaaagataattaccctcctagtttcaaatatgaagattttggaccactatttacagcaaaattttttaatgccaaccagtgggcagatatttttcaggcctctggtgccaaatacattgtcttaacttccaaacatcatgaaggctttaccttgtgggggtcagaatattcgtggaactggaatgccatagatgaggggcccaagagggacattgtcaaggaacttgaggtagccattaggaacagaactgacctgcgttttggactgtactattccctttttgaatggtttcatccgctcttccttgaggatgaatccagttcattccataagcggcaatttccagtttctaagacattgccagagctctatgagttagtgaacaactatcagcctgaggttctgtggtcggatggtgacggaggagcaccggatcaatactggaacagcacaggcttcttggcctggttatataatgaaagcccagttcggggcacagtagtcaccaatgatcgttggggagctggtagcatctgtaagcatggtggcttctatacctgcagtgatcgttataacccaggacatcttttgccacataaatgggaaaactgcatgacaatagacaaactgtcctggggctataggagggaagctggaatctctgactatcttacaattgaagaattggtgaagcaacttgtagagacagtttcatgtggaggaaatcttttgatgaatattgggcccacactagatggcaccatttctgtagtttttgaggagcgactgaggcaagtggggtcctggctaaaagtcaatggagaagctatttatgaaacctatacctggcgatcccagaatgacactgtcaccccagatgtgtggtacacatccaagcctaaagaaaaattagtctatgccatttttcttaaatggcccacatcaggacagctgttccttggccatcccaaagctattctgggggcaacagaggtgaaactactgggccatggacagccacttaactggatttctttggagcaaaatggcattatggtagaactgccacagctaaccattcatcagatgccgtgtaaatggggctgggctctagccctaactaatgtgatctaaagtgcagcagagtggctgatgctgcaagttatgtctaaggctaggaactatcaggtgtctataattgtagcacatggagaaagcaatgtaaactggataagaaaattatttggcagttcagccctttccctttttcccactaaatttttcttaaattacccatgtaaccattttaactctccagtgcactttgccattaaagtctcttcacattgatttgtttccatgtgtgactcagaggtgagaattttttcacattatagtagcaaggaattggtggtattatggaccgaactgaaaattttatgttgaagccatatcccccatgattatatagttatgcatcacttaatatggggatattttctgggaaatgcattgctagtcaatttttttttgtgccaacatcatagagtgtatttacaaaatcctagatggcatagcctactacacacctaatgtgtatggtatagactgttgctcctaggctacagacatatacagcatgttactgaatactgtaggcaatagtaacagtggtatttgtatatcgaaacatatggaaacatagagaaggtacagtaaaaatactgtaaaataaatggtgcacctgtatagggcacttaccacgaatggagcttacaggactggaagttgctctgggtgagtcagtgagtgaatgtgaaggcctaggacattattgaacactgccagacgttataaatactgtatgcttaggctacactacatttataaaaaaaagtttttctttcttcaattataaattaacataagtgtactgtaactttacaaacgttttaatttttaaaacctttttggctcttttgtaataacacttagcttaaaacataaactcattgtgcaaatgtaa

SEQ ID NO: 104 - protein, 467 aa, Homo sapiens
MRPQELPRLAFPLLLLLLLLLPPPPCPAHSATRFDPTWESLDARQLPAWFDQAKFGIFIHWGVFSVPSFGSEWFWWYWQKEKIPKYVEFMKDNYPPSFKYEDFGPLFTAKFFNANQWADIFQASGAKYIVLTSKHHEGFTLWGSEYSWNWNAIDEGPKRDIVKELEVAIRNRTDLRFGLYYSLFEWFHPLFLEDESSSFHKRQFPVSKTLPELYELVNNYQPEVLWSDGDGGAPDQYWNSTGFLAWLYNESPVRGTVVTNDRWGAGSICKHGGFYTCSDRYNPGHLLPHKWENCMTIDKLSWGYRREAGISDYLTIEELVKQLVETVSCGGNLLMNIGPTLDGTISVVFEERLRQVGSWLKVNGEAIYETYTWRSQNDTVTPDVWYTSKPKEKLVYAIFLKWPTSGQLFLGHPKAILGATEVKLLGHGQPLNWISLEQNGIMVELPQLTIHQMPCKWGWALALTNVI

SEQ ID NO: 105 - DNA, 3734 bp, Homo sapiens
gagagggacagaggctggagaaggatgtatggcctgccctgggcttgtctgttccctcctgagcctgagccccttaccttcctgaccccatgaagcacacactggctctgctggctcccctgctgggcctgggcctggggctggccctgagtcagctggctgcaggggccacagactgcaagttccttggcccggcagagcacctgacattcaccccagcagccagggcccggtggctggcccctcgagttcgtgcgccaggactcctggactccctctatggcaccgtgcgccgcttcctctcggtggtgcagctcaatcctttcccttcagagttggtaaaggccctactgaatgagctggcctccgtgaaggtgaatgaggtggtgcggtacgaggcgggctacgtggtatgcgctgtgatcgcgggcctctacctgctgctggtgcccactgccgggctttgcttctgctgctgccgctgccaccggcgctgcgggggacgagtgaagacagagcacaaggcgctggcctgtgagcgcgcggccctcatggtcttcctgctgctgaccaccctcttgctgctgattggtgtggtctgtgcctttgtcaccaaccagcgcacgcatgaacagatgggccccagcatcgaggccatgcctgagaccctgctcagcctctggggcctggtctctgatgtcccccaagagctgcaggccgtggcacagcaattctccctgccccaggagcaagtctcagaggagctggatggtgttggtgtgagcattgggagcgcgatccacactcagctcaggagctccgtgtaccccttgctggcggccgtgggcagtttgggccaggtcctgcaggtctccgtgcaccacctgcaaaccttgaatgctacagtggtagagctgcaggccgggcagcaggacctggagccagccatccgggaacaccgggaccgcctccttgagctgctgcaggaggccaggtgccagggagattgtgcaggggccctgagctgggcccgcaccctggagctgggtgctgacttcagccaggtgccctctgtggaccatgtcctgcaccagctaaaaggtgtccccgaggccaacttctccagcatggtccaggaggagaacagcaccttcaacgcccttccagccctggctgccatgcagacatccagcgtggtgcaagagctgaagaaggcagtggcccagcagccggaaggggtgaggacactggctgaagggttcccgggcttggaggcagcttcccgctgggcccaggcactgcaggaggtggaggagagcagccgcccctacctgcaggaggtgcagagatacgagacctacaggtggatcgtgggctgcgtgctgtgctccgtggtcctattcgtggtgctctgcaacctgctgggcctcaatctgggcatctggggcctgtctgccagggacgaccccagccacccagaagccaagggcgaggctggagcccgcttcctcatggcaggtgtgggcctcagcttcctctttgctgcacccctcatcctcctggtgttcgccaccttcctggtgggtggcaacgtgcagacgctggtgtgccggagctgggagaacggcgagctctttgagtttgcagacaccccagggaacctgcccccgtccatgaacctgtcgcaacttcttggcctgaggaagaacatcagcatccaccaagcctatcagcagtgcaaggaaggggcagcgctctggacagtcctgcagctcaacgactcctacgacctggaggagcacctggatatcaaccagtataccaacaagctacggcaggagttgcagagcctgaaagtagacacacagagcctggacctgctgagctcagccgcccgccgggacctggaggccctgcagagcagtgggcttcagcgcatccactaccccgacttcctcgttcagatccagaggcccgtggtgaagaccagcatggagcagctggcccaggagctgcaaggactggcccaggcccaagacaattctgtgctggggcagcggctgcaggaggaggcccaaggactcagaaaccttcaccaggagaaggtcgtcccccagcagagccttgtggcaaagctcaacctcagcgtcagggccctggagtcctctgccccgaatctccagctggagacctcagatgtcctagccaatgtcacctacctgaaaggagagctgcctgcctgggcagccaggatcctgaggaatgtgagtgagtgtttcctggcccgggagatgggctacttctcccagtacgtggcctgggtgagagaggaggtgactcagcgcattgccacctgccagcccctctccggagccctggacaacagccgtgtgatcctgtgtgacatgatggctgacccctggaatgccttctggttctgcctggcatggtgcaccttcttcctgatccccagcatcatctttgccgtcaagacctccaaatacttccgtcctatccggaaacgcctcagctccaccagctctgaggagactcagctcttccacatcccccgggttacctccctgaagctgtagggccttgtggggtgaggtgaccctgaggctgcctgtcctcccctttgatttagcctgggccacaggacttcggtagctcttgccccagagcccaggctggcatccaggcctggactgtccccagttccggcttacctggccccaccttgcctgctcctttccacccctttctgctcacgacccccatcattcacgctcagaatcacatgggacttctgtgcagctgcagagccagcaagtccctacaggtgtcacccgttacccccatgctggtggcatcctcacaggaagagcctgttctccacctgctggagcctggaccctggggtgggacagaggcctcgtccaaccccactccccttcccgtgtgtcttccccctgccaagcctccccctgccaagcctccccctgcccctctctgagcccctcgccccccacaccgtcctcatctggcctcccccctggcccccacttccctcttatgcccttcctggccctttgcttcctcccttagtcccctcttcaccatatctccactgctaccttgctggccccagagaccaccctgcccaaccaaaccactcaggtaacgccactaatcaggcaggggccaccatggcctaggtctgggctggctgcaggccctgcctcatggcctctgagccctccactgccccagggccttgggccctctgcagatctcatccaggatttattgttgtccagtggggtgagggaggcctgtctgaaggccgagcctccctgcctgcacccaagttagaaatgggggtaccagcacttagcttctctctgagtgctggctcccaaggaagggacctgggacctgggccacagtgggggcttgcccttacctcttcagaaggaagcatcttccacagcccccacccaactttcttaggagtgatctggtggccagaacaggattttgcacggccccttttatcctgcgcatgtggcctagggtcatccccagcccatccctgtgtcagccctgagtgctggacactgcgttccagaaatgaggaagaggagagagaagagatggacagacctcagatccattaaagtgttctcacttcaaaaaaaaaaaaaaaaaaaaaaaaaaaaaaaaaaaaaaaa

SEQ ID NO: 106 - protein, 834 aa, Homo sapiens
MKHTLALLAPLLGLGLGLALSQLAAGATDCKFLGPAEHLTFTPAARARWLAPRVRAPGLLDSLYGTVRRFLSVVQLNPFPSELVKALLNELASVKVNEVVRYEAGYVVCAVIAGLYLLLVPTAGLCFCCCRCHRRCGGRVKTEHKALACERAALMVFLLLTTLLLLIGVVCAFVTNQRTHEQMGPSIEAMPETLLSLWGLVSDVPQELQAVAQQFSLPQEQVSEELDGVGVSIGSAIHTQLRSSVYPLLAAVGSLGQVLQVSVHHLQTLNATVVELQAGQQDLEPAIREHRDRLLELLQEARCQGDCAGALSWARTLELGADFSQVPSVDHVLHQLKGVPEANFSSMVQEENSTFNALPALAAMQTSSVVQELKKAVAQQPEGVRTLAEGFPGLEAASRWAQALQEVEESSRPYLQEVQRYETYRWIVGCVLCSVVLFVVLCNLLGLNLGIWGLSARDDPSHPEAKGEAGARTLMAGVGLSFLFAAPLILLVFATFLVGGNVQTLVCRSWENGELFEFADTPGNLPPSMNLSQLLGLRKNISIHQAYQQCKEGAALWTVLQLNDSYDLEEHLDINQYTNKLRQELQSLKVDTQSLDLLSSAARRDLEALQSSGLQRIHYPDFLVQIQRPVVKTSMEQLAQELQGLAQAQDNSVLGQRLQEEAQGLRNLHQEKVVPQQSLVAKLNLSVRALESSAPNLQLETSDVLANVTYLKGELPAWAARILRNVSECFLAREMGYFSQYVAWVREEVTQRIATCQPLSGALDNSRVILCDMMADPWNAFWFCLAWCTFFLIPSIIFAVKTSKYFRPIRKRLSSTSSEETQLFHIPRVTSLKL

SEQ ID NO: 107 - DNA, 1319 bp, Homo sapiens
gattccgagcgcctccactgctggtccgttggccagatcaactcgccgcgtgggccggccgttccctgagagtctgagcgctcgccgcacccccttccgagcttctattggccgtagcagacgtccgtctgccgctatctccgccccaatacggaagcggcctagtcctccggctccgacagctgggtgtccaggccatggggcagccctgggcggctgggagcacggacggggcgcccgcgcagctgcctctcgtgctcaccgcgctgtgggccgcggccgtgggcctggagctggcttacgtgctggtgctcggtcccgggccgccgccgctgggacccctggcccgggccttgcagctggcgctggccgccttccagctgctcaacctgctgggcaacgtggggctcttcctgcgctcggatcccagcatccgtggcgtgatgctggccggccgcggtctgggccagggctgggcttactgctaccaatgccaaagccaggtgccgccacgcagcggacactgctctgcctgccgcgtctgcatcctgcgtcgggaccaccactgccgcctgctgggccgctgcgtgggcttcggcaactaccggcccttcctgtgcctgctgcttcatgccgccggcgtcctgctccacgtctctgtgctgctgggccctgcactgtcggccctgctgcgagcccacacgcccctccacatggctgccctcctcctgcttccctggctcatgttgctcacaggcagagtgtctctggcacagtttgccttggccttcgtgacggacacgtgcgtggcgggtgcgctgctgtgcggggctgggctgctcttccatgggatgctgctgctgcggggccagaccacatgggagtgggctcggggccagcactcctatgacctgggtccctgccacaacctgcaggcagccctggggccccgctgggccctcgtctggctctggcccttcctggcctccccattgcctggggatgggatcaccttccagaccacagcagatgtgggacacacagcctcctgactccaggaagagccagagctgtgcagggaggaaggggtgagaggggggcccccacacctagactcagtaaggaagtcgggttggaccttaacatctgcattggacaactccaccccttccttggccttgcccctgcccgcctacactcctacgtgtccagggcttgggccgtgacttaggcagaggagtgcagaggagggtctggcaggggctgctcaggccgcctagctgcccctttgccaggttaataaagcactgacttgttaa

SEQ ID NO: 108 - protein, 284 aa, Homo sapiens
MGQPWAAGSTDGAPAQLPLVLTALWAAAVGLELAYVLVLGPGPPPLGPLARALQLALAAFQLLNLLGNVGLFLRSDPSIRGVMLAGRGLGQGWAYCYQCQSQVPPRSGHCSACRVCILRRDHHCRLLGRCVGFGNYRPFLCLLLHAAGVLLHVSVLLGPALSALLRAHTPLHMAALLLLPWLMLLTGRVSLAQFALAFVTDTCVAGALLCGAGLLFHGMLLLRGQTTWEWARGQHSYDLGPCHNLQAALGPRWALVWLWPFLASPLPGDGITFQTTADVGHTAS

SEQ ID NO: 109 - DNA, 1837 bp, Homo sapiens
cttgtctttgtgtcggttgtgattttcctaatctctgattttccttttctctcggacgctctccctcttcggacccattttctcccgtgcttcatgccctgatagcctggccccttcccggcttccttcgctaccggggacgcctctagtttttctgaatttctggctggctccaccctccgcgttcatcttcctcaagagttcgcccctctgggggctcctctgtgtaatcgtcgccttctctgggtatttctgtgaactccgtctcacaccatcccgccatcttctctgccttggccccttttctctgtacagccagctctgtgtccttttcttctccccctctaaaatcgactcctcttctccctgagagccccacctttgtgccccactcctcattttcctacgcctccctctctctgctggtcctctctctccctgcaaggttccattccatcaatttgtttgtcttttgtaggggtggcatcccctctgactactgctccatccttttttttttttttttttttttttttgcttgaggatttcacttcaatcttttctggttgcgtctccacttgtactcagcttgttaggtccaggtccagttgttctgcatctgaggctggcgtgtgctgtcttctctgattggcctaatctccctcacccccgtgagatctgttgtcagccttcgtttctctttcctgtgtcccagcttttctgcgggtcttggcacctttcttggccacagatttctgggttacagagcatgtgtgtctgaggcattgcaggcagaaaagggtggccgacgtgacctctagctggactgctgggcaggggagctgtcctagataaaattggaaagaaacagtgacccagagacaggtggacaaagaattcggggactgatgggaactgagcttgggatccagactgaaactgattccagactgacctctagcacccaggacccagacacagggccatgggaccccagcatttgagacttgtgcagctgttctgccttctaggggccatccccactctgcctcgggctggagctcttttgtgctatgaagcaacagcctcaagattcagagctgttgctttccataactggaagtggcttctgatgaggaacatggtgtgtaagctgcaagagggctgcgaggagacgctagtgttcattgagacagggactgcaaggggagttgtgggctttaaaggctgcagctcgtcttcgtcttaccctgcgcaaatctcctaccttgtttccccacccggagtgtccattgcctcctacagtcgcgtctgccggtcttatctctgcaacaacctcaccaatttggagccttttgtgaaactcaaggccagcactcctaagtctatcacatctgcgtcctgtagctgcccgacctgtgtgggcgagcacatgaaggattgcctcccaaattttgtcaccactaattcttgccccttggctgcttctacgtgttacagttccaccttaaaatttcaggcagggtttctcaataccaccttcctcctcatggggtgtgctcgtgaacataaccagcttttagcagattttcatcatattgggagcatcaaagtgactgaggtcctcaacatcttagagaagtctcagattgttggtgcagcatcctccaggcaagatcctgcttggggtgtcgtcttaggcctcctgtttgccttcagggactgaccatctagctgcacccgacaagcacccagactctttcacataacaaataaaatagcagagttcccttaaaaaaaaaaaaaaaaaaaaaaaaaaaaaaaaaaaaaaaaaaaa

SEQ ID NO: 110 - protein, 246 aa, Homo sapiens
MGPQHLRLVQLFCLLGAIPTLPRAGALLCYEATASRFRAVAFHNWKWLLMRNMVCKLQEGCEETLVFIETGTARGVVGFKGCSSSSSYPAQISYLVSPPGVSIASYSRVCRSYLCNNLTNLEPFVKLKASTPKSITSASCSCPTCVGEHMKDCLPNFVTTNSCPLAASTCYSSTLKFQAGFLNTTFLLMGCAREHNQLLADFHHIGSIKVTEVLNILEKSQIVGAASSRQDPAWGVVLGLLFAFRD

SEQ ID NO: 111 - DNA, 1307 bp, Homo sapiens
ggcagtgcagccgcctcacaggtcggcggacgggccaggcgggcggcctcctgaaccgaaccgaatcggctcctcgggccgtcgtcctcccgcccctcctcgcccgccgccggagttttctttcggtttcttccaagattcctggccttccctcgacggagccgggcccagtgcgggggcgcagggcgcgggagctccacctcctcggctttccctgcgtccagaggctggcatggcgcgggccgagtactgagcgcacggtcggggcacagcagggccggggggtgcagctggctcgcgcctcctctccggccgccgtctcctccggtccctggcgaaagccattgagacaccagctggacgtcacgcgccggagcatgtctgggagtcagagcgaggtggctccatccccgcagagtccgcggagccccgagatgggacgggacttgcggcccgggtcccgcgtgctcctgctcctgcttctgctcctgctggtgtacctgactcagccaggcaatggcaacgagggcagcgtcactggaagttgttattgtggtaaaagaatttcttccgactccccgccatcggttcagttcatgaatcgtctccggaaacacctgagagcttaccatcggtgtctatactacacgaggttccagctcctttcctggagcgtgtgtgggggcaacaaggacccatgggttcaggaattgatgagctgtcttgatctcaaagaatgtggacatgcttactcggggattgtggcccaccagaagcatttacttcctaccagccccccaatttctcaggcctcagagggggcatcttcagatatccacacccctgcccagatgctcctgtccaccttgcagtccactcagcgccccaccctcccagtaggatcactgtcctcggacaaagagctcactcgtcccaatgaaaccaccattcacactgcgggccacagtctggcagctgggcctgaggctggggagaaccagaagcagccggaaaaaaatgctggtcccacagccaggacatcagccacagtgccagtcctgtgcctcctggccatcatcttcatcctcaccgcagccctttcctatgtgctgtgcaagaggaggagggggcagtcaccgcagtcctctccagatctgccggttcattatatacctgtggcacctgactctaatacctgagccaagaatggaagcttgtgagggtaaactgtggcttattcttacaaaaagtgtaataaaggagactgacccctgacaacatggtaggcactgtaaaaaaaaaaaaaa

SEQ ID NO: 112 - protein, 254 aa, Homo sapiens
MGRDLRPGSRVLLLLLLLLLVYLTQPGNGNEGSVTGSCYCGKRISSDSPPSVQFMNRLRKHLRAYHRCLYYTRFQLLSWSVCGGNKDPWVQELMSCLDLKECGHAYSGIVAHQKHLLPTSPPISQASEGASSDIHTPAQMLLSTLQSTQRPTLPVGSLSSDKELTRPNETTIHTAGHSLAAGPEAGENQKQPEKNAGPTARTSATVPVLCLLAIIFILTAALSYVLCKRRRGQSPQSSPDLPVHYIPVAPDSNT

SEQ ID NO: 113 - DNA, 2985 bp, Homo sapiens
gaaaaaaaggaaaggctagcagtcatccaacagaatcatgagacagactttgccttgtatctacttttgggggggccttttgccctttgggatgctgtgtgcatcctccaccaccaagtgcactgttagccatgaagttgctgactgcagccacctgaagttgactcaggtacccgatgatctacccacaaacataacagtgttgaaccttacccataatcaactcagaagattaccagccgccaacttcacaaggtatagccagctaactagcttggatgtaggatttaacaccatctcaaaactggagccagaattgtgccagaaacttcccatgttaaaagttttgaacctccagcacaatgagctatctcaactttctgataaaacctttgccttctgcacgaatttgactgaactccatctcatgtccaactcaatccagaaaattaaaaataatccctttgtcaagcagaagaatttaatcacattagatctgtctcataatggcttgtcatctacaaaattaggaactcaggttcagctggaaaatctccaagagcttctattatcaaacaataaaattcaagcgctaaaaagtgaagaactggatatctttgccaattcatctttaaaaaaattagagttgtcatcgaatcaaattaaagagttttctccagggtgttttcacgcaattggaagattatttggcctctttctgaacaatgtccagctgggtcccagccttacagagaagctatgtttggaattagcaaacacaagcattcggaatctgtctctgagtaacagccagctgtccaccaccagcaatacaactttcttgggactaaagtggacaaatctcactatgctcgatctttcctacaacaacttaaatgtggttggtaacgattcctttgcttggcttccacaactagaatatttcttcctagagtataataatatacagcatttgttttctcactctttgcacgggcttttcaatgtgaggtacctgaatttgaaacggtcttttactaaacaaagtatttcccttgcctcactccccaagattgatgatttttcttttcagtggctaaaatgtttggagcaccttaacatggaagataatgatattccaggcataaaaagcaatatgttcacaggattgataaacctgaaatacttaagtctatccaactcctttacaagtttgcgaactttgacaaatgaaacatttgtatcacttgctcattctcccttacacatattcaacctaaccaagaataaaatctcaaaaatagagagtgatgctttctcttggttgggccacctagaagtacttgacctgggccttaatgaaattgggcaagaactcacaggccaggaatggagaggtctagaaaatattttcgaaatctatctttcctacaacaagtacctgcagctgactaggaactcctttgccttggtcccaagccttcaacgactgatgctccgaagggtggcccttaaaaatgtggatagctctccttcaccattccagcctcttcgtaacttgaccattctggatctaagcaacaacaacatagccaacataaatgatgacatgttggagggtcttgagaaactagaaattctcgatttgcagcataacaacttagcacggctctggaaacacgcaaaccctggtggtcccatttatttcctaaagggtctgtctcacctccacatccttaacttggagtccaacggctttgacgagatcccagttgaggtcttcaaggatttatttgaactaaagatcatcgatttaggattgaataatttaaacacacttccagcatctgtctttaataatcaggtgtctctaaagtcattgaaccttcagaagaatctcataacatccgttgagaagaaggttttcgggccagctttcaggaacctgactgagttagatatgcgctttaatccctttgattgcacgtgtgaaagtattgcctggtttgttaattggattaacgagacccataccaacatccctgagctgtcaagccactacctttgcaacactccacctcactatcatgggttcccagtgagactttttgatacatcatcttgcaaagacagtgccccctttgaactctttttcatgatcaataccagtatcctgttgatttttatctttattgtacttctcatccactttgagggctggaggatatctttttattggaatgtttcagtacatcgagttcttggtttcaaagaaatagacagacagacagaacagtttgaatatgcagcatatataattcatgcctataaagataaggattgggtctgggaacatttctcttcaatggaaaaggaagaccaatctctcaaattttgtctggaagaaagggactttgaggcgggtgtttttgaactagaagcaattgttaacagcatcaaaagaagcagaaaaattatttttgttataacacaccatctattaaaagacccattatgcaaaagattcaaggtacatcatgcagttcaacaagctattgaacaaaatctggattccattatattggttttccttgaggagattccagattataaactgaaccatgcactctgtttgcgaagaggaatgtttaaatctcactgcatcttgaactggccagttcagaaagaacggataggtgcctttcgtcataaattgcaagtagcacttggatccaaaaactctgtacattaaatttatttaaatattcaattagcaaaggagaaactttctcaatttaaaaagttctatggcaaatttaagttttccataaaggtgttataatttgtttattcatatttgtaaatgattatattctatcacaattacatctcttctaggaaaatgtgtctccttatttcaggcctatttttgacaattgacttaattttacccaaaataaaacatataagcacgtaaaaaaaaaa

SEQ ID NO: 114 - protein, 904 aa, Homo sapiens [sequence omitted]

SEQ ID NO: 115 - DNA, 1150 bp, Homo sapiens
gcaggggagctccgagtgtccacaggaagggaactatcagctcctggcatctgtaaggatgctgtccatgctgaggacaatgaccagactctgcttcctgttattcttctctgtggccaccagtgggtgcagtgcagcagcagcctcttctcttgagatgctctcgagggaattcgaaacctgtgccttctccttttcttccctgcctagaagctgcaaagaaatcaaggaacgctgccatagtgcaggtgatggcctgtattttctccgcaccaagaatggtgttgtctaccagaccttctgtgacatgacttctgggggtggcggctggaccctggtggccagcgtgcacgagaatgacatgcgtgggaagtgcacggtgggtgatcgctggtccagtcagcagggcaacaaagcagactacccagagggggatggcaactgggccaactacaacacctttggatctgcagaggcggccacgagcgatgactacaagaaccctggctactacgacatccaggccaaggacctgggcatctggcatgtgcccaacaagtcccccatgcagcattggagaaacagcgccctgctgaggtaccgcaccaacactggcttcctccagagactgggacataatctgtttggcatctaccagaaatacccagtgaaatacagatcagggaaatgttggaatgacaatggcccagccatacctgtggtctatgactttggtgatgctaagaagactgcatcttattactcaccgtatggtcaacgggaatttgttgcaggattcgttcagttccgggtgtttaataacgagagagcagccaacgccctttgtgctgggataaaagttactggctgtaacactgagcatcactgcatcggtggaggagggttcttcccacagggcaaaccccgtcagtgtggggacttctccgcctttgactgggatggatatggaactcacgttaagagcagctgcagtcgggagataacggaggcggctgtactcttgttctatagatgagacagagctctgcggtgtcagggcgagaacccatcttccaaccccggctatttggagacggaaaaactggaattctaacaaggaggagaggagactaaatcacatcaatttgca

SEQ ID NO: 116 - protein, 325 aa, Homo sapiens
MLSMLRTMTRLCFLLFFSVATSGCSAAAASSLEMLSREFETCAFSFSSLPRSCKEIKERCHSAGDGLYFLRTKNGVVYQTFCDMTSGGGGWTLVASVHENDMRGKCTVGDRWSSQQGNKADYPEGDGNWANYNTFGSAEAATSDDYKNPGYYDIQAKDLGIWHVPNKSPMQHWRNSALLRYRTNTGFLQRLGHNLFGIYQKYPVKYRSGKCWNDNGPAIPVVYDFGDAKKTASYYSPYGQREFVAGFVQFRVFNNERAANALCAGIKVTGCNTEHHCIGGGGFFPQGKPRQCGDFSAFDWDGYGTHVKSSCSREITEAAVLLFYR

SEQ ID NO: 117 - DNA, 2329 bp, Homo sapiens
atccctcgacctcgacccacgcgtccgctggaaggtggcgtgccctcctctggctggtaccatgcagctcccactggccctgtgtctcgtctgcctgctggtacacacagccttccgtgtagtggagggccaggggtggcaggcgttcaagaatgatgccacggaaatcatccccgagctcggagagtaccccgagcctccaccggagctggagaacaacaagaccatgaaccgggcggagaacggagggcggcctccccaccacccctttgagaccaaagacgtgtccgagtacagctgccgcgagctgcacttcacccgctacgtgaccgatgggccgtgccgcagcgccaagccggtcaccgagctggtgtgctccggccagtgcggcccggcgcgcctgctgcccaacgccatcggccgcggcaagtggtggcgacctagtgggcccgacttccgctgcatccccgaccgctaccgcgcgcagcgcgtgcagctgctgtgtcccggtggtgaggcgccgcgcgcgcgcaaggtgcgcctggtggcctcgtgcaagtgcaagcgcctcacccgcttccacaaccagtcggagctcaaggacttcgggaccgaggccgctcggccgcagaagggccggaagccgcggccccgcgcccggagcgccaaagccaaccaggccgagctggagaacgcctactagagcccgcccgcgcccctccccaccggcgggcgccccggccctgaacccgcgccccacatttctgtcctctgcgcgtggtttgattgtttatatttcattgtaaatgcctgcaacccagggcagggggctgagaccttccaggccctgaggaatcccgggcgccggcaaggcccccctcagcccgccagctgaggggtcccacggggcaggggagggaattgagagtcacagacactgagccacgcagccccgcctctggggccgcctacctttgctggtcccacttcagaggaggcagaaatggaagcattttcaccgccctggggttttaagggagcggtgtgggagtgggaaagtccagggactggttaagaaagttggataagattcccccttgcacctcgctgcccatcagaaagcctgaggcgtgcccagagcacaagactgggggcaactgtagatgtggtttctagtcctggctctgccactaacttcctgtgtaaccttgaactacacaattctccttcgggacctcaatttccactttgtaaaatgagggtggaggtgggaataggatctcgaggagactattggcatatgattccaaggactccagtgccttttgaatgggcagaggtgagagagagagagagaaagagagagaatgaatgcagttgcattgattcagtgccaaggtcacttccagaattcagagttgtgatgctctcttctgacagccaaagatgaaaaacaaacagaaaaaaaaaagtaaagagtctatttatggctgacatatttacggctgacaaactcctggaagaagctatgctgcttcccagcctggcttccccggatgtttggctacctccacccctccatctcaaagaaataacatcatccattggggtagaaaaggagagggtccgagggtggtgggagggatagaaatcacatccgccccaacttcccaaagagcagcatccctcccccgacccatagccatgttttaaagtcaccttccgaagagaagtgaaaggttcaaggacactggccttgcaggcccgagggagcagccatcacaaactcacagaccagcacatcccttttgagacaccgccttctgcccaccactcacggacacatttctgcctagaaaacagcttcttactgctcttacatgtgatggcatatcttacactaaaagaatattattgggggaaaaactacaagtgctgtacatatgctgagaaactgcagagcataatagctgccacccaaaaatctttttgaaaatcatttccagacaacctcttactttctgtgtagtttttaattgttaaaaaaaaaaagttttaaacagaagcacatgacatatgaaagcctgcaggactggtcgtttttttggcaattcttccacgtgggacttgtccacaagaatgaaagtagtggtttttaaagagttaagttacatatttattttctcacttaagttatttatgcaaaagtttttcttgtagagaatgacaatgttaatattgctttatgaattaacagtctgttcttccagagtccagagacattgttaataaagacaatgaatcatgaaaaaaaaaaaaaaaaaaaaaa

SEQ ID NO: 118 - protein, 213 aa, Homo sapiens
MQLPLALCLVCLLVHTAFRVVEGQGWQAFKNDATEIIPELGEYPEPPPELENNKTMNRAENGGRPPHHPFETKDVSEYSCRELHFTRYVTDGPCRSAKPVTELVCSGQCGPARLLPNAIGRGKWWRPSGPDFRCIPDRYRAQRVQLLCPGGEAPRARKVRLVASCKCKRLTRFHNQSELKDFGTEAARPQKGRKPRPRARSAKANQAELENAY

SEQ ID NO: 119 - DNA, 2906 bp, Homo sapiens
gaggctccgtgccccgccccccgggtgccccgcccctttgcgcggctggcgcggccagccggccaggctcccctcggcaaacctgtctaattggggcggggagcggagcttcctcctctgagggccgtgccgcgctgccagatttgttcttccgcccctgcctccgcggctcggaggcgagcggaaggtgccccggggccgaggcccgtgacggggcgggcgggagccccggcagtccggggtcgccggcgagggccatgtcgctgttgggggacccgctacaggccctgccgccctcggccgcccccacggggccgctgctcgcccctccggccggcgcgaccctcaaccgcctgcgggagccgctgctgcggaggctcagcgagctcctggatcaggcgcccgagggccggggctggaggagactggcggagctggcggggagtcgcgggcgcctccgcctcagttgcctagacctggagcagtgttctcttaaggtactggagcctgaaggaagccccagcctgtgtctgctgaagttaatgggtgaaaaaggttgcacagtcacagaattgagtgatttcctgcaggctatggaacacactgaagttcttcagcttctcagccccccaggaataaagattactgtaaacccagagtcaaaggcagtcttggctggacagtttgtgaaactgtgttgccgggcaactggacatccttttgttcaatatcagtggttcaaaatgaataaagagattccaaatggaaatacatcagagcttatttttaatgcagtgcatgtaaaagatgcaggcttttatgtctgtcgagttaataacaatttcacctttgaattcagccagtggtcacagctggatgtttgcgacatcccagagagcttccagagaagtgttgatggcgtctctgaatccaagttgcaaatctgtgttgaaccaacttcccaaaagctgatgccaggcagcacattggttttacagtgtgttgctgttggaagccctattcctcactaccagtggttcaaaaatgaattaccattaacacatgagaccaaaaagctatacatggtgccttatgtggatttggaacaccaaggaacctactggtgtcatgtatataatgatcgagacagtcaagatagcaagaaggtagaaatcatcataggaagaacagatgaggcagtggagtgcactgaagatgaattaaataatcttggtcatcctgataataaagagcaaacaactgaccagcctttggcgaaggacaaggttgcccttttgataggaaatatgaattaccgggagcaccccaagctcaaagctcctttggtggatgtgtacgaattgactaacttactgagacagctggacttcaaagtggtttcactgttggatcttactgaatatgagatgcgtaatgctgtggatgagtttttactccttttagacaagggagtatatgggttattatattatgcaggacatggttatgaaaattttgggaacagcttcatggtccccgttgatgctccaaatccatataggtctgaaaattgtctgtgtgtacaaaatatactgaaattgatgcaagaaaaagaaactggacttaatgtgttcttattggatatgtgtaggaaaagaaatgactacgatgataccattccaatcttggatgcactaaaagtcaccgccaatattgtgtttggatatgccacgtgtcaaggagcagaagcttttgaaatccagcattctggattggcaaatggaatctttatgaaatttttaaaagacagattattagaagataagaaaatcactgtgttactggatgaagttgcagaagatatgggtaagtgtcaccttaccaaaggcaaacaggctctagagattcgaagtagtttatctgagaagagagcacttactgatccaatacagggaacagaatattctgctgaatctcttgtgcggaatctacagtgggccaaggctcatgaacttccagaaagtatgtgtcttaagtttgactgtggtgttcagattcaattaggatttgcagctgagttttccaatgtcatgatcatctatacaagtatagtttacaaaccaccggagataataatgtgtgatgcctacgttactgattttccacttgatctagatattgatccaaaagatgcaaataaaggcacacctgaagaaactggcagctacttggtatcaaaggatcttcccaagcattgcctctataccagactcagttcactgcaaaaattaaaggaacatctagtcttcacagtatgtttatcatatcagtactcaggattggaagatactgtagaggacaagcaggaagtgaatgttgggaaacctctcattgctaaattagacatgcatcgaggtttgggaaggaagacttgctttcaaacttgtcttatgtctaatggtccttaccagagttctgcagccacctcaggaggagcagggcattatcactcattgcaagacccattccatggtgtttaccattcacatcctggtaatccaagtaatgttacaccagcagatagctgtcattgcagccggactccagatgcatttatttcaagtttcgctcaccatgcttcatgtcattttagtagaagtaatgtgccagtagagacaactgatgaaataccatttagtttctctgacaggctcagaatttctgaaaaatgacctccttgtttttgaaagttagcataattttagatgcctgtgaaatagtactgcacttacataaagtgagacattgtgaaaaggcaaatttgtatatgtagagaaagaatagtagtaactgtttcatagcaaacttcaggactttgagatgttgaaattacattatttaattac

SEQ ID NO: 120 - protein, 824 aa, Homo sapiens
MSLLGDPLQALPPSAAPTGPLLAPPAGATLNRLREPLLRRLSELLDQAPEGRGWRRLAELAGSRGRLRLSCLDLEQCSLKVLEPEGSPSLCLLKLMGEKGCTVTELSDFLQAMEHTEVLQLLSPPGIKITVNPESKAVLAGQFVKLCCRATGHPFVQYQWFKMNKEIPNGNTSELIFNAVHVKDAGFYVCRVNNNFTFEFSQWSQLDVCDIPESFQRSVDGVSESKLQICVEPTSQKLMPGSTLVLQCVAVGSPIPHYQWFKNELPLTHETKKLYMVPYVDLEHQGTYWCHVYNDRDSQDSKKVEIIIGRTDEAVECTEDELNNLGHPDNKEQTTDQPLAKDKVALLIGNMNYREHPKLKAPLVDVYELTNLLRQLDFKVVSLLDLTEYEMRNAVDEFLLLLDKGVYGLLYYAGHGYENFGNSFMVPVDAPNPYRSENCLCVQNILKLMQEKETGLNVFLLDMCRKRNDYDDTIPILDALKVTANIVFGYATCQGAEAFEIQHSGLANGIFMKFLKDRLLEDKKITVLLDEVAEDMGKCHLTKGKQALEIRSSLSEKRALTDPIQGTEYSAESLVRNLQWAKAHELPESMCLKFDCGVQIQLGFAAEFSNVMIIYTSIVYKPPEIIMCDAYVTDFPLDLDIDPKDANKGTPEETGSYLVSKDLPKHCLYTRLSSLQKLKEHLVFTVCLSYQYSGLEDTVEDKQEVNVGKPLIAKLDMHRGLGRKTCFQTCLMSNGPYQSSAATSGGAGHYHSLQDPFHGVYHSHPGNPSNVTPADSCHCSRTPDAFISSFAHHASCHFSRSNVPVETTDEIPFSFSDRLRISEK

SEQ ID NO: 121 - DNA, 2476 bp, Homo sapiens
gcccgcctgaggaagccgtgtgcctgggatgccaagagccagagaatggatcttctccgagtggggacattgctgacaatcccggcttcccgaggcggctaagaacaggcagtttgtgtcggctggctgcagatacccagaggcacaaagagaccgaagccacccggagggacccacggacggacagatggtaggcgcgaacccgagaggaccggcggaggctgagcaccgagagccgccaaggaagagaaactaaccacagccaagttaccccgccggctttccttcgctgcgctaaggaatgaaacccttccagctcgatctgctcttcgtctgcttcttcctcttcagtcaagagctgggcctccagaagagaggatgctgtctggtgctgggctacatggccaaggacaagtttcggagaatgaatgaaggccaagtctattccttcagccagcagccccaggaccaggtggtggtgtcgggacagccagtgacgctactttgcgccatccccgaatacgatggcttcgttctgtggatcaaggacggcttggctctgggtgtgggcagggacctctcaagttacccacagtacctggtggtagggaaccacctgtcaggggagcaccacctgaagatcctgagggcagagctgcaagacgatgcggtgtacgagtgccaggccatccaggccgccatccgctcccgccccgcacgcctcacagtcctggtgccgcctgatgaccccgtcatcctggggggccctgtgatcagcctgcgtgcgggggaccctctcaacctcacctgccacgcagacaatgccaagcctgcagcctccatcatctggttgcgaaagggagaggtcatcaatggggccacctactccaagaccctgcttcgggacggcaagcgggagagcatcgtcagcaccctcttcatctcccctggtgacgtggagaatggccagagcatcgtgtgtcgtgccaccaacaaagccatccccggaggaaaggagacgtcggtcaccattgacatccagcaccctccactggtcaacctctcggtggagccacagccagtgctggaggacaacgtcgtcactttccactgctctgcaaaggccaacccagctgtcacccagtacaggtgggccaagcggggccagatcatcaaggaggcatctggagaggtgtacaggaccacagtggactacacgtacttctcagagcccgtctcctgtgaggtgaccaacgccctgggcagcaccaacctcagccgcacggttgacgtctactttgggccccggatgaccacagaaccccaatccttgctcgtggatctgggctctgatgccatcttcagctgcgcctggaccggcaacccatccctgaccatcgtctggatgaagcggggctccggagtggtcctgagcaatgagaagaccctgaccctcaaatccgtgcgccaggaggacgcgggcaagtacgtgtgccgggctgtggtgccccgtgtgggagccggggagagagaggtgaccctgaccgtcaatggaccccccatcatctccagcacccagacccagcacgccctccacggcgagaagggccagatcaagtgcttcatccggagcacgccgccgccggaccgcatcgcctggtcctggaaggagaacgttctggagtcgggcacatcggggcgctatacggtggagaccatcagcaccgaggagggcgtcatctccaccctgaccatcagcaacatcgtgcgggccgacttccagaccatctacaactgcacggcctggaacagcttcggctccgacactgagatcatccggctcaaggagcaaggttcggaaatgaagtcgggagccgggctggaagcagagtctgtgccgatggccgtcatcattggggtggccgtaggagctggtgtggccttcctcgtccttatggcaaccatcgtggcgttctgctgtgcccgttcccagagaagtacgggagggagatccgggatctcagggagggggacagagaaaaaggccaggcttaggctgccccggagagcaagtaagcaggagtgcaatgaacaggggtcctaacagtgctgtgagctcctggggcagggagtgggtctgatgcatcggtgtatgtgagcctgggcaacatggcgcctggcagagtgggcgctaggctgaggttgacctggactagactgaacttcatctgcagggcagccagcattttggattgaacacatagctctttcagtcaggaactgtacagaaagatagggggaaaagcggtttgtggtttgatccttgctctacaagagctgttagtctagagagaccccatctctacaacaaaataaaaataaagagctgctagtctcaccagaaaagcaggtcactcacacagctgtgggggagtgggtggggaagcaataaaggaattgctttgagaaaacttaa

SEQ ID NO: 122 - protein, 600 aa, Homo sapiens
MKPFQLDLLFVCFFLFSQELGLQKRGCCLVLGYMAKDKFRRMNEGQVYSFSQQPQDQVVVSGQPVTLLCAIPEYDGFVLWIKDGLALGVGRDLSSYPQYLVVGNHLSGEHHLKILRAELQDDAVYECQAIQAAIRSRPARLTVLVPPDDPVILGGPVISLRAGDPLNLTCHADNAKPAASIIWLRKGEVINGATYSKTLLRDGKRESIVSTLFISPGDVENGQSIVCRATNKAIPGGKETSVTIDIQHPPLVNLSVEPQPVLEDNVVTFHCSAKANPAVTQYRWAKRGQIIKEASGEVYRTTVDYTYFSEPVSCEVTNALGSTNLSRTVDVYFGPRMTTEPQSLLVDLGSDAIFSCAWTGNPSLTIVWMKRGSGVVLSNEKTLTLKSVRQEDAGKYVCRAVVPRVGAGEREVTLTVNGPPIISSTQTQHALHGEKGQIKCFIRSTPPPDRIAWSWKENVLESGTSGRYTVETISTEEGVISTLTISNIVRADFQTIYNCTAWNSFGSDTEIIRLKEQGSEMKSGAGLEAESVPMAVIIGVAVGAGVAFLVLMATIVAFCCARSQRSTGGRSGISGRGTEKKARLRLPRRASKQECNEQGS

SEQ ID NO: 123 - DNA, 2893 bp, Homo sapiens
gcgctaggtttcgccggccgcgcggccagatgaggggggcgatggagctggagcctgagctgctgttgcaggaggcccgcgagaacgtggaggcagcgcagagctaccggcgggagctgggtcaccggcttgaggggctgcgtgaggcgcggaggcagatcaaagaaagtgcatcacagacaagggatgttctcaaacagcattttaatgatttaaagggaacccttggaaagctcctggatgagcgattggtgacccttttgcaagaggtggacaccattgaacaggagaccattaaaccactagatgactgccagaagctcatagaacacggagtcaacactgcagaggacttagtccgagaaggtgaaatcgccatgcttggtggtgtgggagaagagaatgagaaactgtggagctttaccaaaaaggcctcgcacattcagttggacagcttaccagaagtacctttactggttgatgtgccttgtttatctgctcagttggatgactcaattcttaacatagtgaaagaccacatttttaagcatggaacagtagcatctcgcccaccagtacagatagaagaactaatagagaaacctggaggcatcattgtacgatggtgtaaggtggatgatgactttacagcccaagattacaggctccagtttcgtaaatgtacttcaaatcattttgaggatgtatatgtaggttctgaaactgaattcatagtattgcacatagaccccaacgttgattaccagttcagagtctgcgcccgaggagatggccgacaggagtggagtccttggagtgtcccccagataggtcattccacattggtgcctcatgagtggacagctggttttgaggggtacagtctgagcagtcgaagaaatatagcacttcggaacgattctgaatcatcgggtgttctctactccagagctccgacttatttctgtgggcagacattaacattcagagttgaaactgtgggacagccagacagaagagatagcataggagtgtgtgcagaaaaacaggatggatatgactctctgcagcgggatcaagctgtgtgcattagtacaaatggtgcagtttttgtcaatggaaaagaaatgacaaatcagttacccgcagttacttctgggtccactgtcacgtttgacattgaagccgtgactctaggaaccaccagtaataatgaaggtggacacttcaagcttcgagtaactataagttcaaataatagagaagtggtttttgactggttacttgatcagtcttgtggttctctttactttggatgctcatttttctatcctggatggaaagtgttagtgttttagatgtttgggtgcttggctttggttttcagggtctaacgtagctgtcctcagcccagcgtagttgtaattgctttaaaaaaaaagttgaattcatctctaacttaggccattggaaagtgtttactggattcattttgtaatattttagcaaaaagagacttcaatgttgtggacaaaatcatacaattcagtcaattttatttttagcatactgttaaatatcacatcacaataaaaatcaaattggcattaaaagtatagaagatataggaagttctatacccttccactgtacttctcaaagttggttattagaccaggaaaacttaatgtaatattatttttaaaatcatcttaaaggatccaagtccatgtaactctttagaacaagaggaataggtcagatagaagaagctgtgtaatgtattaatacatccgttcatgtgctgtccacatgaatgtgttgactgtgctctccatgttcaggtatttgtaagcagtgttgacttttatcccctcttcagtaatctttaagtccccaaaacattataactttttctttttttgttgagacggagtctcactcttgtcactcaggctggagtgcagtggtgtgatctcagttcactgcaacctctgcctcccacgttcaagcgatcctcctgcctcaggctcctgagtagctgggattacaggcgtcggccaccacacctggctaatttttgtatttttagtagagacagggtttcaccatgttggccaggctggtcttgaactcctgacctcaggtgttccacccacctcagccttccaaagtgctaggattacaggtgtgagccactgcagtggaccaatttttgtatttttttaaatgcttgaccaagtgtctaaaataggcaagtagtaccttttgttctttcattgcaatttgatacagtataggctacagtatttttacagggtaaaactagttatggcagtagtatgcacagcagtgttagccatttcatatgtatgtaatatgcacgtgtgtatccaatctgcctgtgacatgcattttactctttgcagagaatgagctgcagtccagtttaatgtcagtttaataagtggtctctttttagcagagcaaaaaaatatagcgtaaatacttattttttttaaaaacttacttaaatttgtgtgtaaaatatttatttgaaggactttaatatgtaaaaataattgtcttctatgccaccctacagattttattttaatgtgaattatttttttcatttcatttttaccagtatagtaaaaaagtagaaaagcaagtatgatatttttgtaagataatttatttggggattcatgttccctgttagacaatcatgaccttttctacttgtctttttatatataagtagtagatttccacttgtttagggattaagaagatgttctagaatgtagataccttgtccagccataggaatcaaaataaaaaactaaaaaaagaaaaaaaaaaaaaaaaaaaaaaaaaaaaaaaaaga

SEQ ID NO: 124 - protein, 442 aa, Homo sapiens
MRGAMELEPELLLQEARENVEAAQSYRRELGHRLEGLREARRQIKESASQTRDVLKQHFNDLKGTLGKLLDERLVTLLQEVDTIEQETIKPLDDCQKLIEHGVNTAEDLVREGEIAMLGGVGEENEKLWSFTKKASHIQLDSLPEVPLLVDVPCLSAQLDDSILNIVKDHIFKHGTVASRPPVQIEELIEKPGGIIVRWCKVDDDFTAQDYRLQFRKCTSNHFEDVYVGSETEFIVLHIDPNVDYQFRVCARGDGRQEWSPWSVPQIGHSTLVPHEWTAGFEGYSLSSRRNIALRNDSESSGVLYSRAPTYFCGQTLTFRVETVGQPDRRDSIGVCAEKQDGYDSLQRDQAVCISTNGAVFVNGKEMTNQLPAVTSGSTVTFDIEAVTLGTTSNNEGGHFKLRVTISSNNREVVFDWLLDQSCGSLYFGCSFFYPGWKVLVF

SEQ ID NO: 125 - DNA, 2037 bp, Homo sapiens
aacatggctgcggcgcctgggctgctcgtctggctgctcgtgctccggctgccctggcgggtgccgggccagctggaccccagcactggccggcggttctcggagcacaaactctgcgcggacgacgaatgcagcatgatgtaccgcggtgaggctcttgaagatttcacaggcccggattgtcgttttgtgaattttaaaaaaggtgatcctgtatatgtttactataaactggcaagaggatggcctgaagtttgggctggaagtgttggacgcacttttggatattttccaaaagatttaatccaggtagttcatgaatataccaaagaagagctacaagttccaacagatgagacggattttgtttgttttgatggaggaagagatgattttcataattataatgtagaagaacttttagggtttttggaactgtacaattctgcagctacagattctgagaaagctgtagaaaaaactttacaggatatggaaaaaaaccctgaattatctaaggaaagggaacctgaacctgaaccagtagaagccaactcagaggaaagtgatagtgtattctcagaaaacactgaggatcttcaggaacagtttacaactcagaagcaccactcccatgcaaacagccaagcaaatcatgctcagggagagcaggcttcatttgaatcttttgaagaaatgctgcaagataaactaaaagtgccagaaagtgaaaacaacaaaaccagcaatagttctcaggtctcaaatgaacaggataagattgatgcctataaacttttgaaaaaagaaatgactctagacttgaaaaccaaatttggctcaacagctgatgcacttgtatctgatgatgagacaaccagactcgttacttcattagaagatgattttgatgaggaattggatactgagtattatgcagttggaaaggaagatgaggagaaccaagaagactttgatgagttgccattacttacctttacagatggggaagatatgaaaactccagcaaagtctggcgttgagaaatatccaacagataaagagcagaattcaaatgaagaggacaaggttcagctaactgtgccccctggcatcaaaaatgatgataaaaatatactaacaacctggggggacactatcttctctattgtcacaggaggtgaagaaacaagagatacgatggatttagagagctctagttcagaggaagaaaaagaagatgatgatgatgcattagtcccagatagcaaacaggggaaaccacagtcagcaacagattatagtgaccctgacaatgtagatgatggtctttttattgtagacattcctaaaacaaataatgacaaagaagtaaacgcagaacatcacattaaaggaaaagggaggggagttcaggaatccaagaggggcctggtacaagatgagacagaattagaggatgaaaatcaagaaggctttaaaacagagcccataaaactatgacctctgaggtttcattggaaagaaagtgtactgtgcattatccattacagtaaaggatttcattggcttcaaaatccaaaagtttattttaaaaggtttgttgttagaactaagctgccttggcagtgtgcatttttgagccaaacaattcaaaaatgtcatttcttccctaaataaaaatcaccttttaagctagagcgtccttacaactttgaaatgtgcaataaagaatacctgtgttttagctaatgtagcatatgtaattgcaaaatgatttagaatgtcatgaaaaatatgaacatttcctgtggaaatgctttaagaacatgtatttccattatcctatttttagtgtacaccagctgaatacggagcaatggtgtttataagcgtttttttaaactatctggtcacaaagactgttacgctaaaaatgtttactaaaagatcactaaactatctcccctcttgctgaagttctttgtagtaatagctcataaaaatttgtttattaatatttaaaaaaaaaaaaaa

SEQ ID NO: 126 - protein, 499 aa, Homo sapiens
MAAAPGLLVWLLVLRLPWRVPGQLDPSTGRRFSEHKLCADDECSMMYRGEALEDFTGPDCRFVNFKKGDPVYVYYKLARGWPEVWAGSVGRTFGYFPKDLIQVVHEYTKEELQVPTDETDFVCFDGGRDDFHNYNVEELLGFLELYNSAATDSEKAVEKTLQDMEKNPELSKEREPEPEPVEANSEESDSVFSENTEDLQEQFTTQKHHSHANSQANHAQGEQASFESFEEMLQDKLKVPESENNKTSNSSQVSNEQDKIDAYKLLKKEMTLDLKTKFGSTADALVSDDETTRLVTSLEDDFDEELDTEYYAVGKEDEENQEDFDELPLLTFTDGEDMKTPAKSGVEKYPTDKEQNSNEEDKVQLTVPPGIKNDDKNILTTWGDTIFSIVTGGEETRDTMDLESSSSEEEKEDDDDALVPDSKQGKPQSATDYSDPDNVDDGLFIVDIPKTNNDKEVNAEHHIKGKGRGVQESKRGLVQDETELEDENQEGFKTEPIKL

SEQ ID NO: 127 - DNA, 925 bp, Homo sapiens
cagcgcgcgaggctccgtgagtgtgtctcctgcgcgctgagaggcggggggaggcggaggaccaggaggaggaggaggaggaggaggagggggagaatgcccggagccgccgccgctgccgccgccgccgccgccgcgatgctcccggctcaggaggctgccaagctgtaccacaccaactatgtgcggaactcgcgggccatcggcgtgctgtgggccatcttcaccatctgctttgccatcgtcaacgtggtgtgcttcatccagccctactggataggcgacggcgtggacaccccgcaagccggctatttcgggctcttccactactgcatcggcaacggcttctcccgggagctgacctgcaggggcagcttcacggacttctccacgctgccctcgggcgccttcaaagccgcctccttctttatcggcctctccatgatgctcatcattgcctgcatcatttgctttaccctcttcttcttctgcaacacggccactgtgtacaagatatgtgcctggatgcagctcacctccgctgcctgccttgtgcttggctgtatgattttccctgatggctgggactcagatgaagtaaaacggatgtgtggagaaaagacagacaagtacactcttggggcttgctcagtccgctgggcatacatcctggctattattggaattttggatgccctgatcctctcatttctagcatttgtgcttggtaatcgacaagacagcttgatggcagaggaactgaaggcagaaaacaaagttctgctaagccaatattctctagaatgagcacaaaacaaatcgaataacagctaaacaaatcgaataacagctaaacgaatcgaataacagcttttgtacatcaacatcaagaaggaatacgcctgagagagatcagagtatatag

SEQ ID NO: 128 - protein, 236 aa, Homo sapiens
MPGAAAAAAAAAAAMLPAQEAAKLYHTNYVRNSRAIGVLWAIFTICFAIVNVVCFIQPYWIGDGVDTPQAGYFGLFHYCIGNGFSRELTCRGSFTDFSTLPSGAFKAASFFIGLSMMLIIACIICFTLFFFCNTATVYKICAWMQLTSAACLVLGCMIFPDGWDSDEVKRMCGEKTDKYTLGACSVRWAYILAIIGILDALILSFLAFVLGNRQDSLMAEELKAENKVLLSQYSLE

SEQ ID NO: 129 - DNA, 1218 bp, Homo sapiens
acacaacttcagctgaggaacttggcacggccagcttgggacccaggaccctaacgcagaggcgctgtgtttggaagtcccgctatcacggccccccagatggggcctggacgatgcctcctgacggccttgttgcttctggccctggcgccaccgccggaagcctcccagtactgcggccgccttgaatactggaacccagacaacaagtgctgcagcagctgcctgcaacgcttcgggccgcccccctgcccggactatgagttccgggaaaactgcggactcaatgaccacggcgatttcgtaacgcccccgttccgaaagtgttcttctgggcagtgcaaccccgacggcgcggagctatgtagcccctgcggcggcggagccgtgacccctactcccgccgcgggcgggggcagaaccccgtggcgctgcagagagaggccggtccctgccaaggggcactgccccctcacacctggaaacccaggcgcccctagctcccaggagcgcagctcaccagcaagttccattgcctggaggacccctgagcctgtccctcagcaggcctggccgaatttccttccgctcgtggtgctggtcctgctcctgaccttggcggtgatagcgatcctcctgtttattctgctctggcatctctgctggcccaaggagaaagccgacccctatccctatcctggcttggtctgcggagtccccaacacccacaccccttcctcctcgcatctgtcctccccaggcgccctggagacaggggacacatggaaggaggcctcactacttccactcctgagcagggaactgtccagtctggcgtcacaacccctgtctcgcctcctggatgagctggaggtgctggaagagctgattgtactgctggaccctgagcctgggccaggtgggggtatggcccatggcactactcgacacctggccgcaagatatgggctgcctgctgcctggtccacctttgcctattcgctgaggccgagtcgctcgccgctgcgggctctgattgagatggtggtggcaagggagccctctgcctccctgggccagcttggcacacacctcgcccagctagggcgggcagatgcattgcgggtgctgtccaagcttggctcatctggggtttgctgggcttaacacccaataaagaactttgctgactactaaaaaaaaaaaaaaaaaaaaaaa

SEQ ID NO: 130 - protein, 355 aa, Homo sapiens
MGPGRCLLTALLLLALAPPPEASQYCGRLEYWNPDNKCCSSCLQRFGPPPCPDYEFRENCGLNDHGDFVTPPFRKCSSGQCNPDGAELCSPCGGGAVTPTPAAGGGRTPWRCRERPVPAKGHCPLTPGNPGAPSSQERSSPASSIAWRTPEPVPQQAWPNFLPLVVLVLLLTLAVIAILLFILLWHLCWPKEKADPYPYPGLVCGVPNTHTPSSSHLSSPGALETGDTWKEASLLPLLSRELSSLASQPLSRLLDELEVLEELIVLLDPEPGPGGGMAHGTTRHLAARYGLPAAWSTFAYSLRPSRSPLRALIEMVVAREPSASLGQLGTHLAQLGRADALRVLSKLGSSGVCWA

SEQ ID NO: 131 - DNA, 2425 bp, Homo sapiens
atgtggtggagagttctcagcttgctggcatggttccccttgcaagaggcctctctgactaaccacacagaaaccatcaccgtggaggaaggccagacgctcactctaaagtgtgtcacttctctgaggaagaactcctccctccagtggctgaccccctcagggttcaccatttttttaaatgagtatcctgctttaaaaaattccaaataccagcttcttcatcactcggccaatcagctctccatcactgtgcctaacgtaaccctgcaagatgaaggcgtgtacaagtgcttacattacagcgactctgtaagcacaaaggaagtgaaagtgattgtgctggcaactcctttcaagccaatcctggaagcttcagttatcagaaagcaaaatggagaagaacatgttgtactcatgtgctccaccatgagaagcaagccccctccgcagataacctggctacttgggaatagcatggaagtgtccggtggaacgctccatgaatttgaaactgatgggaagaaatgtaatactaccagcactctcataatccacacttatggcaaaaattcaacggtggactgcattatccgacacagaggcctgcaagggagaaaactagtagcacccttccggtttgaagatttggttactgatgaagagacagcttcagatgctctggagagaaactctctatcctctcaagacccacagcagcccaccagtactgtctcagtaacggaagattctagtacatcggagattgacaaggaagagaaagaacaaaccactcaagatcctgacttgaccaccgaagcaaatcctcagtatttaggactggcaagaaagaaaagtggcatcctgctgctcacgctggtgtccttcctcattttcatactcttcatcatagtccagctcttcatcatgaagctgaggaaagcacatgtgatatggaagagagaaaacgaagtttcagaacacacactagaaagttacagatcaaggtcaaataatgaagaaacatcatctgaagagaaaaatggccaatcttcccaccctatgcgttgcatgaactacatcacaaagttgtactcagaagcaaaaacaaagaggaaggaaaatgtacaacattcaaaattagaagaaaagcacatccaagtaccagagagtattgtgtagtgctctctgcaatggaacatgtgatttcagggttgccgcagtgtcacctcagtggaccagcctgggggaaggagcttaattgctgagacattaataatgacctcttagtgcaatgcaagatggtgtcctcggataatgatctgccccggagctagggcagcaacatgaggaccaaaccatgcacataaagcttgtagtttaaaaaagaaaagcaaaaaaataattatgcctgacactacttcagagcaggaggattctacgaagccttggggatcagggtcagtgtgagcagctaacatcctacctcaaatggaacaggattttttgatgctttgctctaatggaactgttttaaaaatttttttttctttttaatattttcttctggtcacaaaataaagaaatttgggatgcaaagtacctaaagatctctgatcctaagaagttacttctggccaggcgcggtggctcatgcctgtaatcctagcactttgggaggctgaggtaggcagatcacttgaggtcaggagttggagaccagcctggccaacatagtgaaaccccgtctctactaaaaatgcaaaaattagccaggcgtagtggtgcgcacctgtagtctcagatacttgggaggctgagggtggagaatcgcttgaacctgggaggtggagattgcagtgagtcaagatctcaccactgaactccagcctgggcgacagagggagactctgtctcaaaaaaaaagaagttacttccaagacagacttttaacatgtaaccagcacaaagcaatgtcagggaggagtgatacatgacaataaatgagtcagatgagcaagaaggccccagaacccatgccccaaggcacaaagaggagctcagggtaggcccaaagattcgaattccttagattactaaatcacatgagcggaccttgtctgtcagataagatttttacctggaaattccatgaccaatacatgtgcaaaagaaaatgatgggttgaatttactgattattgacctaatggatggctttttaaaatgttttaataaaaagcagaatagatgtttgtttttctagtggttataccaagctatacttcctgttttcacgtgtgaaagtaacatgggacatgcctttcttttccgatcagtttatttaagctatacagcaaactttggcatttatgtggagcatttctcattgttggaatctgaataaaccaattacaaaataaaaa

SEQ ID NO: 132 - protein, 393 aa, Homo sapiens
MWWRVLSLLAWFPLQEASLTNHTETITVEEGQTLTLKCVTSLRKNSSLQWLTPSGFTIFLNEYPALKNSKYQLLHHSANQLSITVPNVTLQDEGVYKCLHYSDSVSTKEVKVIVLATPFKPILEASVIRKQNGEEHVVLMCSTMRSKPPPQITWLLGNSMEVSGGTLHEFETDGKKCNTTSTLIIHTYGKNSTVDCIIRHRGLQGRKLVAPFRFEDLVTDEETASDALERNSLSSQDPQQPTSTVSVTEDSSTSEIDKEEKEQTTQDPDLTTEANPQYLGLARKKSGILLLTLVSFLIFILFIIVQLFIMKLRKAHVIWKRENEVSEHTLESYRSRSNNEETSSEEKNGQSSHPMRCMNYITKLYSEAKTKRKENVQHSKLEEKHIQVPESIV

SEQ ID NO: 133 - DNA, 480 bp, Homo sapiens
gttaaccagcgcagtcctccgtgcgtcccgcccgccgctgccctcactcccggccaggatggcatcctgtctggccctgcgcatggcgctgctgctggtctccggggttctggcccctgcggtgctcacagacgatgttccacaggagcccgtgcccacgctgtggaacgagccggccgagctgccgtcgggagaaggccccgtggagagcaccagccccggccgggagcccgtggacaccggtcccccagcccccaccgtcgcgccaggacccgaggacagcaccgcgcaggagcggctggaccagggcggcgggtcgctggggcccggcgctatcgcggccatcgtgatcgccgccctgctggccacctgcgtggtgctggcgctcgtggtcgtcgcgctgagaaagttttctgcctcctgaagcgaataaaggggccgcgcccggccgcggcgcgactcggcaaaaaaaaaaaaaaa

SEQ ID NO: 134 - protein, 121 aa, Homo sapiens
MASCLALRMALLLVSGVLAPAVLTDDVPQEPVPTLWNEPAELPSGEGPVESTSPGREPVDTGPPAPTVAPGPEDSTAQERLDQGGGSLGPGAIAAIVIAALLATCVVLALVVVALRKFSAS

SEQ ID NO: 135 - DNA, 845 bp, Homo sapiens
ggagcctcctaatgcagtcttctgcacagtcctggggactgactgactgaatcacacctctggggctgggggctgctgacatgtgtgcctttccttggctgcttcttctcctgctgctccaggagggcagccaaaggagactctggagatggtgtggatccgaggaagtggttgcggtccttcaggagtccatcagcctccccctggaaataccaccagatgaagaggttgagaacatcatctggtcctctcacaaaagtcttgccactgtggtgccagggaaagagggacatccagctaccatcatggtgaccaatccacactaccagggccaagtgagcttcctggaccccagctattccctgcatatcagcaatctgagctgggaggattcagggctttaccaagctcaagtcaacctgagaacatcccagatctctaccatgcagcagtacaatctatgtgtctaccatcctaactatgcttctgagaagccttcaacagccttctgcctcctggccaagggattgctcatcttcttgctcttggtaattctggccatgggactctgggtcatccgagtccagaaaagacacaaaatgccaaggatgaagaaactcatgagaaacagaatgaaattgaggaaggaggcaaagcctggctccagccctgcctgactgctccttgggaaccccagtcctgagcttggtttcttcccagcacccagagaatccttcctcagctctcttctttccaggggaaggaggtgctcaggggtgggtatccagagagccatacttctgagggaagactggctggcaataaagtcaaattaagtgaccaca

SEQ ID NO: 136 - protein, 198 aa, Homo sapiens [sequence omitted]

SEQ ID NO: 137 - DNA, 2714 bp, Homo sapiens
cggctcgaggccctttgtgagggctgtgagctgcgcctgacggtggcaccatgagcagctcaggtggggcgcccggggcgtccgccagctctgcgccgcccgcgcaggaagagggcatgacgtggtggtaccgctggctgtgtcgcctgtctggggtgctgggggcagtctcttgcgcgatctctggcctcttcaactgcatcaccatccaccctctgaacatcgcggccggcgtgtggatgatcatgaatgccttcatcttgttgctgtgtgaggcgcccttctgctgccagttcatcgagtttgcaaacacagtggcggagaaggtggaccggctgcgctcctggcagaaggctgtcttctactgcgggatggcggtcgttcccatcgtcatcagcctgaccctgaccacgctgctgggcaacgccatcgcctttgctacgggggtgctgtacggactctctgctctgggcaaaaagggcgatgcgatctcctatgccaggatccagcagcagaggcagcaggcggatgaggagaagctcgcggagaccctggagggggagctgtgaagggctgggcgcccctccctccctgtcccctcttctggctctgtgtgggtccaagtgaggcctggactgtccacgctgaggcacagcctggagaggggcctttgcacgtgtccctacacctggagtcctctgctcctttctccagactggcttaagccaggagccactggctgctggtgtgagggtctgggctgctggacttgaggcagagcctgcagcagctgtgtggacactacccagccctactcctctgctgggtgggtctgcagatctcacaccacagacagggctgcctgtgacctgctgtgacctgggagcagcttcccctggagatgctggtcctggcttgaggggaggggcaagtgggaccctgccacctgggcactgagcagagggacctcccccagctctcttagcaggtggagccccagggcctgggacagcctgccgctgccagcaacctcccactgctgcctagggtgcagcgcccactgtcaccctgccttctgaagaagcccacagggctcctaaggtgcaccccggtacctggaactgcagccttggcagtgactggacagctgggtgggggatgctccctgctggccctgggaaccttggacaggccacctcaaggcccctcggctgcccctcctccctgggcctgctggggcccctaggttctacccatcaccccccgcccctgctggccttggtgctaaggaagtggggagagcaggctctccctggcaccgagggtgcccaccctctccctggtgtggccccgtcaacatcagccacagcccagccccattagtgggttagtgggtctgacctcagccccactcaggtgctcctgctggcctgcccaagccctgccctcagggagcttctgccttttaagaactgggcagaggccacagtcacctccccacacagagctgtccccactgccctgggtgccaggctgtccggagccaggcctacccagggaggatgcagagagctggtgcccaggatgtgcacccccatattccctctgccctgtggcctcagcccgctggcctctctgaccgtgaggctggctctcagccatcgggcaggtgcctggtcaggcctggcttagcccaggtggggcttggcagaagcgggcgggtgtggaagatattccatctggggccaaccccaggctgggcctgcgctgagcttctggagcgcaggtactgggtcttgctaagtgaactgtttcccaggaacacctctcgggcccatctgcgtctgaggctgggagtggcatctgaggccgggagtggcatctgaggccaggagtggcaggctggtgggctgggcgtggggttttctgggccctgcccagtactgccctggggacttggtgggctcctgggtcagcagcatcccacccctgggagtctggccagctgagccccagggtggcaggggcattatagcctggtggacatgtgccttcagggttcctccggggccaccttcctcaggccagtgctgggttcaaagggctgtgtgtgtgtgtgtttgtgtgtgtatgtatatgtgtgtgggtgcacacatctgtcccatgtatgcagtgagacctgtctacctcccacaaggagcaagggctctgcccgccctctgctcattcctacccaggtagtgggaccccgggcccccttctgcctggcttgcctgcttctgccctttccagaggggtctcactgacagccagagacagcaggagaagggttggctgtggatcaaggaaggctgcccctgtaccctgtggggaaatggtgggtgcatggctggatgcagaggtggaaggccctgggccacaggcgagagtgggcgtgtcacctgtcccaggttcccagcaagtctgcagctgtgcagtcctggggtccctgaccctgtcgcccagggggcgtgctgtccagcaggggccctgccttgcaaggaacgtctctccggcggctgggccgctcctgcctggtctgggctgtgtgtggcgccctttcctccttgtttgttcctctgtgttctgtgtgcgtcttaagcaataaagcgtggccgtgggaaaaaaaaaaaaaaaaaaaaaaaaaaaaaaaaaaaaaaaaaaaaaaaaaaaaa

SEQ ID NO: 138 - protein, 172 aa, Homo sapiens
MSSSGGAPGASASSAPPAQEEGMTWWYRWLCRLSGVLGAVSCAISGLFNCITIHPLNIAAGVWMIMNAFILLLCEAPFCCQFIEFANTVAEKVDRLRSWQKAVFYCGMAVVPIVISLTLTTLLGNAIAFATGVLYGLSALGKKGDAISYARIQQQRQQADEEKLAETLEGEL

SEQ ID NO: 139 - DNA, 3334 bp, Homo sapiens
cggctcgagctcgagccgaatcggctcgaggggcagtggagcacccagcaggccgccaacatgctctgtctgtgcctgtacgtgccggtcatcggggaagcccagaccgagttccagtactttgagtcgaaggggctccctgccgagctgaagtccattttcaagctcagtgtcttcatcccctcccaggaattctccacctaccgccagtggaagcagaaaattgtacaagctggagataaggaccttgatgggcagctagactttgaagaatttgtccattatctccaagatcatgagaagaagctgaggctggtgtttaagattttggacaaaaagaatgatggacgcattgacgcgcaggagatcatgcagtccctgcgggacttgggagtcaagatatctgaacagcaggcagaaaaaattctcaagagcatggataaaaacggcacgatgaccatcgactggaacgagtggagagactaccacctcctccaccccgtggaaaacatccccgagatcatcctctactggaagcattccacgatctttgatgtgggtgagaatctaacggtcccggatgagttcacagtggaggagaggcagacggggatgtggtggagacacctggtggcaggaggtggggcaggggccgtatccagaacctgcacggcccccctggacaggctcaaggtgctcatgcaggtccatgcctcccgcagcaacaacatgggcatcgttggtggcttcactcagatgattcgagaaggaggggccaggtcactctggcggggcaatggcatcaacgtcctcaaaattgcccccgaatcagccatcaaattcatggcctatgagcagatcaagcgccttgttggtagtgaccaggagactctgaggattcacgagaggcttgtggcagggtccttggcaggggccatcgcccagagcagcatctacccaatggaggtcctgaagacccggatggcgctgcggaagacaggccagtactcaggaatgctggactgcgccaggaggatcctggccagagagggggtggccgccttctacaaaggctatgtccccaacatgctgggcatcatcccctatgccggcatcgaccttgcagtctacgagacgctcaagaatgcctggctgcagcactatgcagtgaacagcgcggaccccggcgtgtttgtgctcctggcctgtggcaccatgtccagtacctgtggccagctggccagctaccccctggccctagtcaggacccggatgcaggcgcaagcctctattgagggcgctccggaggtgaccatgagcagcctcttcaaacatatcctgcggaccgagggggccttcgggctgtacagggggctggcccccaacttcatgaaggtcatcccagctgtgagcatcagctacgtggtctacgagaacctgaagatcaccctgggcgtgcagtcgcggtgacggggggagggccgcccggcagtggactcgctgatcctgggccgcagcctggggtgtgcagccatctcattctgtgaatgtgccaacactaagctgtctcgagccaagctgtgaaaaccctagacgcacccgcagggagggtggggagagctggcaggcccagggcttgtcctgctgaccccagcagaccctcctgttggttccagcgaagaccacaggcattccttagggtccagggtcagcaggctccgggctcacatgtgtaaggacaggacattttctgcagtgcctgccaatagtgagcttggagcctggaggccggcttagttcttccatttcacccttgcagccagctgttggccacggcccctgccctctggtctgccgtgcatctccctgtgccctcttgctgcctgcctgtctgctgaggtaaggtgggaggagggctacagcccacatcccaccccctcgtccaatcccataatccatgatgaaaggtgaggtcacgtggcctcccaggcctgacttcccaacctacagcattgacgccaacttggctgtgaaggaagaggaaaggatctggccttgtggtcactggcatctgagccctgctgatggctggggctctcgggcatgcttgggagtgcagggggctcgggctgcctggcctggctgcacagaaggcaagtgctggggctcatggtgctctgagctggcctggaccctgtcaggatgggccccacctcagaaccaaactcactgtccccactgtggcatgagggcagtggagcaccatgtttgagggcgaagggcagagcgtttgtgtgttctggggagggaaggaaaaggtgttggaggccttaattatggactgttgggaaaagggttttgtccagaaggacaagccggacaaatgagcgacttctgtgcttccagaggaagacgagggagcaggagcttggctgactgctcagagtctgttctgacgccctgggggttcctgtccaaccccagcaggggcgcagcgggaccagccccacattccacttgtgtcactgcttggaacctatttattttgtatttatttgaacagagttatgtcctaactatttttatagatttgtttaattaatagcttgtcattttcaagttcattttttattcatatttatgttcatggttgattgtaccttcccaagcccgcccagtgggatgggaggaggaggagaaggggggccttgggccgctgcagtcacatctgtccagagaaattccttttgggactggaggcagaaaagcggccagaaggcagcagccctggctcctttcctttggcaggttggggaagggcttgcccccagccttaggatttcagggtttgactgggggcgtggagagagagggaggaacctcaataaccttgaaggtggaatccagttatttcctgcgctgcgagggtttctttatttcactcttttctgaatgtcaaggcagtgaggtgcctctcactgtgaatttgtggtgggcgggggctggaggagagggtggggggctggctccgtccctcccagccttctgctgcccttgcttaacaatgccggccaactggcgacctcacggttgcacttccattccaccagaatgacctgatgaggaaatcttcaataggatgcaaagatcaatgcaaaaattgttatatatgaacatataactggagtcgtcaaaaagcaaattaagaaagaattggacgttagaagttgtcatttaaagcagccttctaataaagttgtttcaaagctgaaaaaaaaaaaaaaaaaaaaaaaaaaaaaaaaaaaaaaaaaaaaaaaaaaaaaaaaaaaaaaaa

SEQ ID NO: 140 - protein, 469 aa, Homo sapiens
MLCLCLYVPVIGEAQTEFQYFESKGLPAELKSIFKLSVFIPSQEFSTYRQWKQKIVQAGDKDLDGQLDFEEFVHYLQDHEKKLRLVFKILDKKNDGRIDAQEIMQSLRDLGVKISEQQAEKILKSMDKNGTMTIDWNEWRDYHLLHPVENIPEIILYWKHSTIFDVGENLTVPDEFTVEERQTGMWWRHLVAGGGAGAVSRTCTAPLDRLKVLMQVHASRSNNMGIVGGFTQMIREGGARSLWRGNGINVLKIAPESAIKFMAYEQIKRLVGSDQETLRIHERLVAGSLAGAIAQSSIYPMEVLKTRMALRKTGQYSGMLDCARRILAREGVAAFYKGYVPNMLGIIPYAGIDLAVYETLKNAWLQHYAVNSADPGVFVLLACGTMSSTCGQLASYPLALVRTRMQAQASIEGAPEVTMSSLFKHILRTEGAFGLYRGLAPNFMKVIPAVSISYVVYENLKITLGVQSR

SEQ ID NO: 141 - DNA, 1734 bp, Homo sapiens
gtggactctgagaagcccaggcagttgaggacaggagagagaaggctgcagacccagagggagggaggacagggagtcggaaggaggaggacagaggagggcacagagacgcagagcaagggcggcaaggaggagaccctggtgggaggaagacactctggagagagagggggctgggcagagatgaagttccaggggcccctggcctgcctcctgctggccctctgcctgggcagtggggaggctggccccctgcagagcggagaggaaagcactgggacaaatattggggaggcccttggacatggcctgggagacgccctgagcgaaggggtgggaaaggccattggcaaagaggccggaggggcagctggctctaaagtcagtgaggcccttggccaagggaccagagaagcagttggcactggagtcaggcaggttccaggctttggcgcagcagatgctttgggcaacagggtcggggaagcagcccatgctctgggaaacactgggcacgagattggcagacaggcagaagatgtcattcgacacggagcagatgctgtccgcggctcctggcagggggtgcctggccacagtggtgcttgggaaacttctggaggccatggcatctttggctctcaaggtggccttggaggccagggccagggcaatcctggaggtctggggactccgtgggtccacggataccccggaaactcagcaggcagctttggaatgaatcctcagggagctccctggggtcaaggaggcaatggagggccaccaaactttgggaccaacactcagggagctgtggcccagcctggctatggttcagtgagagccagcaaccagaatgaagggtgcacgaatcccccaccatctggctcaggtggaggctccagcaactctgggggaggcagcggctcacagtcgggcagcagtggcagtggcagcaatggtgacaacaacaatggcagcagcagtggtggcagcagcagtggcagcagcagtggcagcagcagtggcggcagcagtggcggcagcagtggtggcagcagtggcaacagtggtggcagcagaggtgacagcggcagtgagtcctcctggggatccagcaccggctcctcctccggcaaccacggtgggagcggcggaggaaatggacataaacccgggtgtgaaaagccagggaatgaagcccgcgggagcggggaatctgggattcagggcttcagaggacagggagtttccagcaacatgagggaaataagcaaagagggcaatcgcctccttggaggctctggagacaattatcgggggcaagggtcgagctggggcagtggaggaggtgacgctgttggtggagtcaatactgtgaactctgagacgtctcctgggatgtttaactttgacactttctggaagaattttaaatccaagctgggtttcatcaactgggatgccataaacaaggaccagagaagctctcgcatcccgtgacctccagacaaggagccaccagattggatgggagcccccacactccctccttaaaacaccaccctctcatcactaatctcagcccttgcccttgaaataaaccttagctgccccacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 142 - protein, 440 aa, Homo sapiens
MKFQGPLACLLLALCLGSGEAGPLQSGEESTGTNIGEALGHGLGDALSEGVGKAIGKEAGGAAGSKVSEALGQGTREAVGTGVRQVPGFGAADALGNRVGEAAHALGNTGHEIGRQAEDVIRHGADAVRGSWQGVPGHSGAWETSGGHGIFGSQGGLGGQGQGNPGGLGTPWVHGYPGNSAGSFGMNPQGAPWGQGGNGGPPNFGTNTQGAVAQPGYGSVRASNQNEGCTNPPPSGSGGGSSNSGGGSGSQSGSSGSGSNGDNNNGSSSGGSSSGSSSGSSSGGSSGGSSGGSSGNSGGSRGDSGSESSWGSSTGSSSGNHGGSGGGNGHKPGCEKPGNEARGSGESGIQGFRGQGVSSNMREISKEGNRLLGGSGDNYRGQGSSWGSGGGDAVGGVNTVNSETSPGMFNFDTFWKNFKSKLGFINWDAINKDQRSSRIP

SEQ ID NO: 143 - DNA, 2340 bp, Homo sapiens
gggctgttgatttgtgggggattttgaagagaggaggaataggaggaaggggttgaggggctgcctctggcatatgcacacactcacacattctgtcacacccgtcacacacacataccatgttctccatccccccaggtccagccctcagtgctgtcccatccagcagggctaccctgaagctctggctgcagccctcccgtccagtgggcaggcggcttcatccctcctttctctcccaaagcccaactgctgtcactgcatgctctgccaaggaggagggaactgcagtgacagcaggagtaagagtgggaggcaggacagagctgggacacaggtatggagagggggttcagcgagcctagagagggcagactatcagggtgccggcggtgagaatccagggagaggagcggaaacagaagaggggcagaagaccggggcacttgtgggttgcagagcccctcagccatgttgggagccaagccacactggctaccaggtcccctacacagtcccgggctgcccttggttctggtgcttctggccctgggggccgggtgggcccaggaggggtcagagcccgtcctgctggagggggagtgcctggtggtctgtgagcctggccgagctgctgcaggggggcccgggggagcagccctgggagaggcaccccctgggcgagtggcatttgctgcggtccgaagccaccaccatgagccagcaggggaaaccggcaatggcaccagtggggccatctacttcgaccaggtcctggtgaacgagggcggtggctttgaccgggcctctggctccttcgtagcccctgtccggggtgtctacagcttccggttccatgtggtgaaggtgtacaaccgccaaactgtccaggtgagcctgatgctgaacacgtggcctgtcatctcagcctttgccaatgatcctgacgtgacccgggaggcagccaccagctctgtgctactgcccttggaccctggggaccgagtgtctctgcgcctgcgtcgggggaatctactgggtggttggaaatactcaagtttctctggcttcctcatcttccctctctgaggacccaagtctttcaagcacaagaatccagcccctgacaactttcttctgccctctcttgccccagaaacagcagaggcaggagagagactccctctggctcctatcccacctctttgcatgggaccctgtgccaaacacccaagtttaagagaagagtagagctgtggcatctccagaccaggcctttccacccacccacccccagttaccctcccagccacctgctgcatctgttcctgcctgcagccctaggatcagggcaaggtttggcaagaaggaagatctgcactactttgcggcctctgctcctccggttcccccaccccagcttcctgctcaatgctgatcagggacaggtggcgcaggtgagcctgacaggcccccacaggagcccagatggacaagcctcagcgtaccctgcaggcttcttcctgtgaggaaagccagcatcacggatctcagccagcaccgtcagaagctgagccagcaccgtatgggctagggtgggaggctcagccacaggcagaagggtgggaagggcctggagtctgtggctggtgaggaaggaaggagggtgtattgtctagactgaacatggtacacattctgcatgtatagcagagcagccagcaggtagcaatcctggctgtccttctatgctggatcccagatggactctggcccttacctccccacctgagattagggtgagtgtgtttgctctggctgagagcagagctgagagcaggtatacagagctggaagtggaccatggaaaacatcgataaccatgcatcctcttgcttggccacctcctgaaactgctccacctttgaagtttgaactttagtccctccacactctgactgctgcctccttcctcccagctctctcactgagttatcttcactgtacctgttccagcatatccccactatctctctttctcctgatctgtgctgtcttattctcctccttaggcttcctattacctgggattccatgattcattccttcagaccctctcctgccagtatgctaaaccctccctctctctttcttatcccgctgtcccattggcccagcctggatgaatctatcaataaaacaactagagaatggtggtcagtgagacactatagaattactaaggagaagatgcctctggagtttggatcgggtgttacaggtacaagtaggtatgttgcagaggaaaataaatatcaaactgtatactaaaattaaaaa

SEQ ID NO: 144 - protein, 205 aa, Homo sapiens
MLGAKPHWLPGPLHSPGLPLVLVLLALGAGWAQEGSEPVLLEGECLVVCEPGRAAAGGPGGAALGEAPPGRVAFAAVRSHHHEPAGETGNGTSGAIYFDQVLVNEGGGFDRASGSFVAPVRGVYSFRFHVVKVYNRQTVQVSLMLNTWPVISAFANDPDVTREAATSSVLLPLDPGDRVSLRLRRGNLLGGWKYSSFSGFLIFPL

SEQ ID NO: 145 - DNA, 1197 bp, Homo sapiens
cagcagtggtctctcagtcctctcaaagcaaggaaagagtactgtgtgctgagagaccatggcaaagaatcctccagagaattgtgaagactgtcacattctaaatgcagaagcttttaaatccaagaaaatatgtaaatcacttaagatttgtggactggtgtttggtatcctggccctaactctaattgtcctgttttgggggagcaagcacttctggccggaggtacccaaaaaagcctatgacatggagcacactttctacagcaatggagagaagaagaagatttacatggaaattgatcctgtgaccagaactgaaatattcagaagcggaaatggcactgatgaaacattggaagtgcacgactttaaaaacggatacactggcatctacttcgtgggtcttcaaaaatgttttatcaaaactcagattaaagtgattcctgaattttctgaaccagaagaggaaatagatgagaatgaagaaattaccacaactttctttgaacagtcagtgatttgggtcccagcagaaaagcctattgaaaaccgagattttcttaaaaattccaaaattctggagatttgtgataacgtgaccatgtattggatcaatcccactctaatatcagtttctgagttacaagactttgaggaggagggagaagatcttcactttcctgccaacgaaaaaaaagggattgaacaaaatgaacagtgggtggtccctcaagtgaaagtagagaagacccgtcacgccagacaagcaagtgaggaagaacttccaataaatgactatactgaaaatggaatagaatttgatcccatgctggatgagagaggttattgttgtatttactgccgtcgaggcaaccgctattgccgccgcgtctgtgaacctttactaggctactacccatatccatactgctaccaaggaggacgagtcatctgtcgtgtcatcatgccttgtaactggtgggtggcccgcatgctggggagggtctaataggaggtttgagctcaaatgcttaaactgctggcaacatataataaatgcatgctattcaatgaatttctgcctatgaggcatctggcccctggtagccagctctccagaattacttgtaggtaattcctctcttcatgttctaataaacttctacattatcaccaaaaaaaaaaaaaaaaaaa

SEQ ID NO: 146 - protein, 317 aa, Homo sapiens
MAKNPPENCEDCHILNAEAFKSKKICKSLKICGLVFGILALTLIVLFWGSKHFWPEVPKKAYDMEHTFYSNGEKKKIYMEIDPVTRTEIFRSGNGTDETLEVHDFKNGYTGIYFVGLQKCFIKTQIKVIPEFSEPEEEIDENEEITTTFFEQSVIWVPAEKPIENRDFLKNSKILEICDNVTMYWINPTLISVSELQDFEEEGEDLHFPANEKKGIEQNEQWVVPQVKVEKTRHARQASEEELPINDYTENGIEFDPMLDERGYCCIYCRRGNRYCRRVCEPLLGYYPYPYCYQGGRVICRVIMPCNWWVARMLGRV

SEQ ID NO: 147 - DNA, 2207 bp, Homo sapiens
  Unsure 2153,2160: Unknown base
cacgcctcccgctgccagcccggcaccgggatcttaatcagtcactatgaaaactcattagctccacagcaatgagtcctccactgctgaagcttggcgctgtgcttagtaccatggcaatgatctcaaactggatgtcccaaactctcccatccttggtgggactgaacaccacgaggctgtcgactccggataccttaactcagattagtcctaaagaagggtggcaggtgtacagctcagctcaggatcctgatgggcggtgcatttgcacagttgttgctccagaacaaaacctgtgttcccgggatgccaaaagcaggcaacttcgccaactactggaaaaggttcagaacatgtcccagtctattgaagtcttaaacttgagaactcagagagatttccaatatgttttaaaaatggaaacccaaatgaaagggctgaaggcaaaatttcggcagattgaagatgatcgaaagacacttatgaccaagcattttcaggagttgaaagagaaaatggacgagctcctgcctttgatccccgtgctggaacagtacaaaacagatgctaagttaatcacccagttcaaggaggaaataaggaatctgtctgctgtcctcactggtattcaggaggaaattggtgcctatgactacgaggaactacaccaaagagtgctgagcttggaaacaagacttcgtgactgcatgaaaaagctaacatgtggcaaactgatgaaaatcacaggcccagttacagtcaagacatctggaacccgatttggtgcttggatgacagaccctttagcatctgagaaaaacaacagagtctggtacatggacagttatactaacaataaaattgttcgtgaatacaaatcaattgcagactttgtcagtggggctgaatcaaggacatacaaccttcctttcaagtgggcaggaactaaccatgttgtctacaatggctcactctattttaacaagtatcagagtaatatcatcatcaaatacagctttgatatggggagagtgcttgcccaacgaagcctggagtatgctggttttcataatgtttacccctacacatggggtggattctctgacatcgacctaatggctgatgaaatcgggctgtgggctgtgtatgcaactaaccagaatgcaggcaatattgtcatcagccaacttaaccaagataccttggaggtgatgaagagctggagcactggctaccccaagagaagtgcaggggaatctttcatgatctgtgggacactgtatgtcaccaactcccacttaactggagccaaggtgtattattcctattccaccaaaacctccacatatgagtacacagacattcccttccataaccaatactttcacatatccatgcttgactacaatgcaagagatcgagctctctatgcctggaacaatggccaccaggtgctgttcaatgtcacccttttccatatcatcaagacagaggatgacacataggcaaatgtgacatgttttcattgatttaaacagtgtgatttgtgataaactctataagaccccttccgtttttttcttcactattatttttcatcatttctccaaagcaaagcatttttattgtaaagttggtgtttcaaaaacatagctgagcttgtctaacttaccatgttggaaacacatcttaacttctaaatttacaaggcctatcatgtccttgtcatgaaaagcactaaaaaaaaaaaagagtttaagtggctaaagtcatagttttgcaagagattaatgatctgccttatattagagtcagagactaatggtggcttaaatgcacgaatgtctttttttttaaaactgtcattttttactgtcttttgctccatctcaggaaatattttggtaggaattaggagaacaaaaagcacttttatcccatttatttctttaaaaaatgtaaggatttcatttatattgaaaaataatattaatcattttgctgttaacacaattctctgatgcggtgctgtacagtcatttttaaatctcttgctaacattttattggcagtatgtatttctaccattgtaaccaccattgtgctattgtatctcttcacttctgtgaaagtaatattttttataaaanacactgnaattttaaaaaaaaaaaaaaaaaacaaaaaaaaaaaaaaaaaaaaaa

SEQ ID NO: 148 - protein, 478 aa, Homo sapiens
MSPPLLKLGAVLSTMAMISNWMSQTLPSLVGLNTTRLSTPDTLTQISPKEGWQVYSSAQDPDGRCICTVVAPEQNLCSRDAKSRQLRQLLEKVQNMSQSIEVLNLRTQRDFQYVLKMETQMKGLKAKFRQIEDDRKTLMTKHFQELKEKMDELLPLIPVLEQYKTDAKLITQFKEEIRNLSAVLTGIQEEIGAYDYEELHQRVLSLETRLRDCMKKLTCGKLMKITGPVTVKTSGTRFGAWMTDPLASEKNNRVWYMDSYTNNKIVREYKSIADFVSGAESRTYNLPFKWAGTNHVVYNGSLYFNKYQSNIIIKYSFDMGRVLAQRSLEYAGFHNVYPYTWGGFSDIDLMADEIGLWAVYATNQNAGNIVISQLNQDTLEVMKSWSTGYPKRSAGESFMICGTLYVTNSHLTGAKVYYSYSTKTSTYEYTDIPFHNQYFHISMLDYNARDRALYAWNNGHQVLFNVTLFHIIKTEDDT

SEQ ID NO: 149 - DNA, 2343 bp, Homo sapiens
aacgaagcgtgcgcgctttggtaaccggctagaaatcccgcacgcgcgcctgcctcctctccccaggcctgagctgcccctcccactgcctttccttcttcccgcgagtcagaagcttcgcgagggcccagagaggcggtggggtgggcgaccctacgccagctccgggcgggagaaagcccaccctctcccgcgccccaggaaaccgccggcgttcggcgctgcgcagagccatggaattctcctggctggagacgcgctgggcgcggcccttttacctggcgttcgtgttctgcctggccctggggctgctgcaggccattaagctgtacctgcggaggcagcggctgctgcgggacctgcgccccttcccagcgccccccacccactggttccttgggcaccagaagtttattcaggatgataacatggagaagcttgaggaaattattgaaaaataccctcgtgccttccctttctggattgggccctttcaggcatttttctgtatctatgacccagactatgcaaagacacttctgagcagaacagatcccaagtcccagtacctgcagaaattctcacctccacttcttggaaaaggactagcggctctagacggacccaagtggttccagcatcgtcgcctactaactcctggattccattttaacatcctgaaagcatacattgaggtgatggctcattctgtgaaaatgatgctggataagtgggagaagatttgcagcactcaggacacaagcgtggaggtctatgagcacatcaactcgatgtctctggatataatcatgaaatgcgctttcagcaaggagaccaactgccagacaaacagcacccatgatccttatgcaaaagccatatttgaactcagcaaaatcatatttcaccgcttgtacagtttgttgtatcacagtgacataattttcaaactcagccctcagggctaccgcttccagaagttaagccgagtgttgaatcagtacacagatacaataatccaggaaagaaagaaatccctccaggctggggtaaagcaggataacactccgaagaggaagtaccaggattttctggatattgtcctttctgccaaggatgaaagtggtagcagcttctcagatattgatgtacactctgaagtgagcacattcctgttggcaggacatgacaccttggcagcaagcatctcctggatcctttactgcctggctctgaaccctgagcatcaagagagatgccgggaggaggtcaggggcatcctgggggatgggtcttctatcacttgggaccagctgggtgagatgtcgtacaccacaatgtgcatcaaggagacgtgccgattgattcctgcagtcccgtccatttccagagatctcagcaagccacttaccttcccagatggatgcacattgcctgcagggatcaccgtggttcttagtatttggggtcttcaccacaaccctgctgtctggaaaaacccaaaggtctttgaccccttgaggttctctcaggagaattctgatcagagacacccctatgcctacttaccattctcagctggatcaaggaactgcattgggcaggagtttgccatgattgagttaaaggtaaccattgccttgattctgctccacttcagagtgactccagaccccaccaggcctcttactttccccaaccattttatcctcaagcccaagaatgggatgtatttgcacctgaagaaactctctgaatgttagatctcagggtacaatgattaaacgtactttgtttttcgaagttaaatttacagctaatgatccaagcagatagaaagggatcaatgtatggtgggaggattggaggttggtgggataggggtctctgtgaagagatccaaaatcatttctaggtacacagtgtgtcagctagatctgtttctatataactttgggagattttcagatcttttctgttaaactttcactactattaatgctgtatacaccaatagactttcatatattttctgttgtttttaaaatagttttcagaattatgcaagtaataagtgcatgtatgctcactgtcaaaaattcccaacactagaaaatcatgtagaataaaaattttaaatctcacttcacttagccgacattccatgccctgaccaatcctactgcttttcctaaaaacagaataatttggtgtgcattctttcagactttttcctatacattttatatgtagaaatgtagcaatgtatttgtatagatgtgatcattcctatattgttattgatttttttcacttaataaaaattcaccttattccttaaaa

SEQ ID NO: 150 - protein, 509 aa, Homo sapiens
MEFSWLETRWARPFYLAFVFCLALGLLQAIKLYLRRQRLLRDLRPFPAPPTHWFLGHQKFIQDDNMEKLEEIIEKYPRAFPFWIGPFQAFFCIYDPDYAKTLLSRTDPKSQYLQKFSPPLLGKGLAALDGPKWFQHRRLLTPGFHFNILKAYIEVMAHSVKMMLDKWEKICSTQDTSVEVYEHINSMSLDIIMKCAFSKETNCQTNSTHDPYAKAIFELSKIIFHRLYSLLYHSDIIFKLSPQGYRFQKLSRVLNQYTDTIIQERKKSLQAGVKQDNTPKRKYQDFLDIVLSAKDESGSSFSDIDVHSEVSTFLLAGHDTLAASISWILYCLALNPEHQERCREEVRGILGDGSSITWDQLGEMSYTTMCIKETCRLIPAVPSISRDLSKPLTFPDGCTLPAGITVVLSIWGLHHNPAVWKNPKVFDPLRFSQENSDQRHPYAYLPFSAGSRNCIGQEFAMIELKVTIALILLHFRVTPDPTRPLTFPNHFILKPKNGMYLHLKKLSEC

SEQ ID NO: 151 - DNA, 43 bp, Artificial sequence
  Oligonucleotide Probe
tgtaaaacgacggccagttaaatagacctgcaattattaatct

SEQ ID NO: 152 - DNA, 41 bp, Artificial sequence
  Oligonucleotide Probe
caggaaacagctatgaccacctgcacacctgcaaatccatt

SEQ ID NO: 153 - DNA, 20 bp, Artificial sequence
  Oligonucleotide Probe
ccacgttggcttgaaattga

SEQ ID NO: 154 - DNA, 50 bp, Artificial sequence
  Oligonucleotide Probe
cctttagaattgatcaagacaattcatgatttgattctctatctccagag

SEQ ID NO: 155 - DNA, 19 bp, Artificial sequence
  Oligonucleotide Probe
tcgtctaacatagcaaatc

SEQ ID NO: 156 - DNA, 20 bp, Artificial sequence
  Oligonucleotide Probe
gtgcagcagagtggcttaca

SEQ ID NO: 157 - DNA, 20 bp, Artificial sequence
  Oligonucleotide Probe
actggaccaattcttctgtg

SEQ ID NO: 158 - DNA, 45 bp, Artificial sequence
  Oligonucleotide Probe
gatattctagcatattgtcagaaggaaggatggtgcaaattagct

SEQ ID NO: 159 - DNA, 21 bp, Artificial sequence
  Oligonucleotide Probe
ttcagcttctgggatgtaggg

SEQ ID NO: 160 - DNA, 24 bp, Artificial sequence
  Oligonucleotide Probe
tattcctaccatttcacaaatccg

SEQ ID NO: 161 - DNA, 49 bp, Artificial sequence
  Oligonucleotide Probe
ggaggactgtgccaccatgagagactcttcaaacccaaggcaaaattgg

SEQ ID NO: 162 - DNA, 20 bp, Artificial sequence
  Oligonucleotide Probe
caacaatgagggcaccaagc

SEQ ID NO: 163 - DNA, 24 bp, Artificial sequence
  Oligonucleotide Probe
gatggctaggttctggaggttctg

SEQ ID NO: 164 - DNA, 47 bp, Artificial sequence
  Oligonucleotide Probe
caacctgcaggagattgacctcaaggacaacaacctcaagaccatcg

SEQ ID NO: 165 - DNA, 24 bp, Artificial sequence
  Oligonucleotide Probe
tggaaggagatgcgatgccacctg

SEQ ID NO: 166 - DNA, 20 bp, Artificial sequence
  Oligonucleotide Probe
tgaccagtggggaaggacag

SEQ ID NO: 167 - DNA, 20 bp, Artificial sequence
  Oligonucleotide Probe
acagagcagagggtgccttg

SEQ ID NO: 168 - DNA, 24 bp, Artificial sequence
  Oligonucleotide Probe
tcagggacaagtggtgtctctccc

SEQ ID NO: 169 - DNA, 24 bp, Artificial sequence
  Oligonucleotide Probe
tcagggaaggagtgtgcagttctg

SEQ ID NO: 170 - DNA, 50 bp, Artificial sequence
  Oligonucleotide Probe
acagctcccgatctcagttacttgcatcgcggacgaaatcggcgctcgct

SEQ ID NO: 171 - DNA, 22 bp, Artificial sequence
  Oligonucleotide Probe
aacaaggtaagatgccatcctg

SEQ ID NO: 172 - DNA, 24 bp, Artificial sequence
  Oligonucleotide Probe
aaacttgtcgatggagaccagctc

SEQ ID NO: 173 - DNA, 45 bp, Artificial sequence
  Oligonucleotide Probe
aggggctgcaaagcctggagagcctctccttctatgacaaccagc

SEQ ID NO: 174 - DNA, 24 bp, Artificial sequence
  Oligonucleotide Probe
caacgtgatttcaaagctgggctc

SEQ ID NO: 175 - DNA, 20 bp, Artificial sequence
  Oligonucleotide Probe
gcctcgtatcaagaatttcc

SEQ ID NO: 176 - DNA, 18 bp, Artificial sequence
  Oligonucleotide Probe
agtggaagtcgacctccc

SEQ ID NO: 177 - DNA, 24 bp, Artificial sequence
  Oligonucleotide Probe
ctcacctgaaatctctcatagccc

SEQ ID NO: 178 - DNA, 50 bp, Artificial sequence
  Oligonucleotide Probe
cgcaaaacccattttgggagcaggaattccaatcatgtctgtgatggtgg

SEQ ID NO: 179 - DNA, 25 bp, Artificial sequence
  Oligonucleotide Probe
gggatgcaggtggtgtctcatgggg

SEQ ID NO: 180 - DNA, 18 bp, Artificial sequence
  Oligonucleotide Probe
ccctcatgtaccggctcc

SEQ ID NO: 181 - DNA, 18 bp, Artificial sequence
  Oligonucleotide Probe
gtgtgacacagcgtgggc

SEQ ID NO: 182 - DNA, 18 bp, Artificial sequence
  Oligonucleotide Probe
gaccggcaggcttctgcg

SEQ ID NO: 183 - DNA, 25 bp, Artificial sequence
  Oligonucleotide Probe
cagcagcttcagccaccaggagtgg

SEQ ID NO: 184 - DNA, 24 bp, Artificial sequence
  Oligonucleotide Probe
ctgagccgtgggctgcagtctcgc

SEQ ID NO: 185 - DNA, 45 bp, Artificial sequence
  Oligonucleotide Probe
ccgactacgactggttcttcatcatgcaggatgacacatatgtgc

SEQ ID NO: 186 - DNA, 24 bp, Artificial sequence
  Oligonucleotide Probe
aggaacttctggatcgggctcacc

SEQ ID NO: 187 - DNA, 24 bp, Artificial sequence
  Oligonucleotide Probe
gggtctgggccaggtggaagagag

SEQ ID NO: 188 - DNA, 45 bp, Artificial sequence
  Oligonucleotide Probe
gccaaggactccttccgctgggccacaggggagcaccaggccttc

SEQ ID NO: 189 - DNA, 23 bp, Artificial sequence
  Oligonucleotide Probe
ctgagaacgcgcctgaaactgtg

SEQ ID NO: 190 - DNA, 22 bp, Artificial sequence
  Oligonucleotide Probe
agcgttgtcattgacatcggcg

SEQ ID NO: 191 - DNA, 50 bp, Artificial sequence
  Oligonucleotide Probe
ttagttgctccattcaggaggatctacccttcctcctgaaatccgcggaa

SEQ ID NO: 192 - DNA, 23 bp, Artificial sequence
  Oligonucleotide Probe
gggtggatggtactgctgcatcc

SEQ ID NO: 193 - DNA, 26 bp, Artificial sequence
  Oligonucleotide Probe
tgttgtgctgtgggaaatcagatgtg

SEQ ID NO: 194 - DNA, 46 bp, Artificial sequence
  Oligonucleotide Probe
gtgtctggaggctgtggccgttttgttttcttgggctaaaatcggg

SEQ ID NO: 195 - DNA, 24 bp, Artificial sequence
  Oligonucleotide Probe
cctgggctctggctcttctttcag

SEQ ID NO: 196 - DNA, 24 bp, Artificial sequence
  Oligonucleotide Probe
ccactcagaggcctcagcttttcc

SEQ ID NO: 197 - DNA, 45 bp, Artificial sequence
  Oligonucleotide Probe
tttcggccacccaggcacggaaaggcttctgggactacttcagcc

SEQ ID NO: 198 - DNA, 24 bp, Artificial sequence
  Oligonucleotide Probe
tgcggagatcctactggcacaggg

SEQ ID NO: 199 - DNA, 18 bp, Artificial sequence
  Oligonucleotide Probe
cgagttagtcagagcatg

SEQ ID NO: 200 - DNA, 18 bp, Artificial sequence
  Oligonucleotide Probe
cagatggtgctgttgccg

SEQ ID NO: 201 - DNA, 29 bp, Artificial sequence
  Oligonucleotide Probe
caactggaacaggaactgagatgtggatc

SEQ ID NO: 202 - DNA, 24 bp, Artificial sequence
  Oligonucleotide Probe
ctggttcagcagtgcaagggtctg

SEQ ID NO: 203 - DNA, 18 bp, Artificial sequence
  Oligonucleotide Probe
cctctccgattaaaacgc

SEQ ID NO: 204 - DNA, 45 bp, Artificial sequence
  Oligonucleotide Probe
gagaggactggttgccatggcaaatgctggttctcatgataatgg

SEQ ID NO: 205 - DNA, 24 bp, Artificial sequence
  Oligonucleotide Probe
gcaggacttctacgacttcaaggc

SEQ ID NO: 206 - DNA, 24 bp, Artificial sequence
  Oligonucleotide Probe
agtctgggccaggtacttgaaggc

SEQ ID NO: 207 - DNA, 50 bp, Artificial sequence
  Oligonucleotide Probe
caacatccggggcaaactggtgtcgctggagaagtaccgcggatcggtgt

SEQ ID NO: 208 - DNA, 23 bp, Artificial sequence
  Oligonucleotide Probe
aggcttcgctgcgactagacctc

SEQ ID NO: 209 - DNA, 24 bp, Artificial sequence
  Oligonucleotide Probe
ccaggtcgggtaaggatggttgag

SEQ ID NO: 210 - DNA, 50 bp, Artificial sequence
  Oligonucleotide Probe
tttctacgcattgattccatgtttgctcacagatgaagtggccattctgc

SEQ ID NO: 211 - DNA, 24 bp, Artificial sequence
  Oligonucleotide Probe
tcgattatggacgaacatggcagc

SEQ ID NO: 212 - DNA, 20 bp, Artificial sequence
  Oligonucleotide Probe
ttctgagatccctcatcctc

SEQ ID NO: 213 - DNA, 24 bp, Artificial sequence
  Oligonucleotide Probe
aggttcagggacagcaagtttggg

SEQ ID NO: 214 - DNA, 50 bp, Artificial sequence
  Oligonucleotide Probe
tttgctggacctcggctacggaattggcttccctctacggacagctggat

SEQ ID NO: 215 - DNA, 25 bp, Artificial sequence
  Oligonucleotide Probe
ctcggggaaagggacttgatgttgg

SEQ ID NO: 216 - DNA, 26 bp, Artificial sequence
  Oligonucleotide Probe
gcgaaggtgagcctctatctcgtgcc

SEQ ID NO: 217 - DNA, 19 bp, Artificial sequence
  Oligonucleotide Probe
cagcctacacgtattgagg

SEQ ID NO: 218 - DNA, 48 bp, Artificial sequence
  Oligonucleotide Probe
cagtcagtacaatcctggcataatatacggccaccatgatgcagtccc

SEQ ID NO: 219 - DNA, 24 bp, Artificial sequence
  Oligonucleotide Probe
gttctcaatgagctacccgtcccc

SEQ ID NO: 220 - DNA, 24 bp, Artificial sequence
  Oligonucleotide Probe
cgcgatgtagtggaactcgggctc

SEQ ID NO: 221 - DNA, 50 bp, Artificial sequence
  Oligonucleotide Probe
atccgcataaaccctcagtcctggtttgataatgggagcatctgcatgag

SEQ ID NO: 222 - DNA, 18 bp, Artificial sequence
  Oligonucleotide Probe
gtaaagtcgctggccagc

SEQ ID NO: 223 - DNA, 18 bp, Artificial sequence
  Oligonucleotide Probe
cccgatctgcctgctgta

SEQ ID NO: 224 - DNA, 24 bp, Artificial sequence
  Oligonucleotide Probe
ctgcactgtatggccattattgtg

SEQ ID NO: 225 - DNA, 45 bp, Artificial sequence
  Oligonucleotide Probe
cagaaacccatgataccctactgaacaccgaatcccctggaagcc

SEQ ID NO: 226 - DNA, 24 bp, Artificial sequence
  Oligonucleotide Probe
gtgccactacggggtgtggacgac

SEQ ID NO: 227 - DNA, 24 bp, Artificial sequence
  Oligonucleotide Probe
tcccatttcttccgtggtgcccag

SEQ ID NO: 228 - DNA, 46 bp, Artificial sequence
  Oligonucleotide Probe
ccagaagaagtccttcatgatgctcaagtacatgcacgaccactac

SEQ ID NO: 229 - DNA, 24 bp, Artificial sequence
  Oligonucleotide Probe
tccttcggctgctgtgatcagcac

SEQ ID NO: 230 - DNA, 24 bp, Artificial sequence
  Oligonucleotide Probe
cccaggtggcggttgagatagtcg

SEQ ID NO: 231 - DNA, 45 bp, Artificial sequence
  Oligonucleotide Probe
cgctgcccggtactgggacatcatggaatattttgatctgaagag

SEQ ID NO: 232 - DNA, 24 bp, Artificial sequence
  Oligonucleotide Probe
atcctccaacatggagcctcttgc

SEQ ID NO: 233 - DNA, 20 bp, Artificial sequence
  Oligonucleotide Probe
gtatcttgtcaaccctgagg

SEQ ID NO: 234 - DNA, 24 bp, Artificial sequence
  Oligonucleotide Probe
taaccagagctgctatgtcaggcc

SEQ ID NO: 235 - DNA, 50 bp, Artificial sequence
  Oligonucleotide Probe
aggcaaagtttcactagttgtaaacgtggccagtgactgccaactcacag

SEQ ID NO: 236 - DNA, 24 bp, Artificial sequence
  Oligonucleotide Probe
gctttcattgccacgtggagtatg

SEQ ID NO: 237 - DNA, 23 bp, Artificial sequence
  Oligonucleotide Probe
acctagtgaggctgggatttggc

SEQ ID NO: 238 - DNA, 40 bp, Artificial sequence
  Oligonucleotide Probe
ccgcctggctctgtgccaagcccttcaaagtcatctgtat

SEQ ID NO: 239 - DNA, 22 bp, Artificial sequence
  Oligonucleotide Probe
ccgccagaagaatgcagttctg

SEQ ID NO: 240 - DNA, 22 bp, Artificial sequence
  Oligonucleotide Probe
cctccaccctcagaactgcctc

SEQ ID NO: 241 - DNA, 40 bp, Artificial sequence
  Oligonucleotide Probe
gcaattggagcagtggagaaagacgtgggcctgtcggatg

SEQ ID NO: 242 - DNA, 23 bp, Artificial sequence
  Oligonucleotide Probe
tggtttgaccaggccaagttcgg

SEQ ID NO: 243 - DNA, 24 bp, Artificial sequence
  Oligonucleotide Probe
ggattcatcctcaaggaagagcgg

SEQ ID NO: 244 - DNA, 24 bp, Artificial sequence
  Oligonucleotide Probe
aacttgcagcatcagccactctgc

SEQ ID NO: 245 - DNA, 45 bp, Artificial sequence
  Oligonucleotide Probe
ttccgtgcccagcttcggtagcgagtggttctggtggtattggca

SEQ ID NO: 246 - DNA, 23 bp, Artificial sequence
  Oligonucleotide Probe
ccagcatttgagacttgtgcagc

SEQ ID NO: 247 - DNA, 25 bp, Artificial sequence
  Oligonucleotide Probe
gactgtaggaggcaatggacactcc

SEQ ID NO: 248 - DNA, 41 bp, Artificial sequence
  Oligonucleotide Probe
ccatctccactctgcccgggctggagctcttttgtgctatg

SEQ ID NO: 249 - DNA, 21 bp, Artificial sequence
  Oligonucleotide Probe
atgcagctcccactggccctg

SEQ ID NO: 250 - DNA, 27 bp, Artificial sequence
  Oligonucleotide Probe
ctagtaggcgttctccagctcggcctg

SEQ ID NO: 251 - DNA, 40 bp, Artificial sequence
  Oligonucleotide Probe
cttccgctgcatccccgaccgctaccgcgcgcagcgcgtg

SEQ ID NO: 252 - DNA, 22 bp, Artificial sequence
  Oligonucleotide Probe
catcgtgtgtcgtgccaccaac

SEQ ID NO: 253 - DNA, 23 bp, Artificial sequence
  Oligonucleotide Probe
ctctggccattctccacgtcacc

SEQ ID NO: 254 - DNA, 43 bp, Artificial sequence
  Oligonucleotide Probe
ggaaaggagacgtcggtcaccattgacatccagcaccctccac

SEQ ID NO: 255 - DNA, 19 bp, Artificial sequence
  Oligonucleotide
gctgctcgtgctccggctg

SEQ ID NO: 256 - DNA, 23 bp, Artificial sequence
  Oligonucleotide Probe
cacaaaacgacaatccgggcctg

SEQ ID NO: 257 - DNA, 43 bp, Artificial sequence
  Oligonucleotide Probe
gcacaaactctgcgcggacgacgaatgcagcatgttaatgtac

SEQ ID NO: 258 - DNA, 24 bp, Artificial sequence
  Oligonucleotide Probe
aggcagccaccagctctgtgctac

SEQ ID NO: 259 - DNA, 27 bp, Artificial sequence
  Oligonucleotide Probe
cagagagggaagatgaggaagccagag

SEQ ID NO: 260 - DNA, 42 bp, Artificial sequence
  Oligonucleotide Probe
ctgtgctactgcccttggaccctggggaccgagtgtctctgc

SEQ ID NO: 261 - DNA, 28 bp, Artificial sequence
  Oligonucleotide Probe
cggcagattgaagatgatcgaaagacac

SEQ ID NO: 262 - DNA, 29 bp, Artificial sequence
  Oligonucleotide Probe
gtcttgtttccaagctcagcactctttgg

SEQ ID NO: 263 - DNA, 45 bp, Artificial sequence
  Oligonucleotide Probe
tcaggagttgaaagagaaaatggacgagctcctgcctttgatccc

SEQ ID NO: 264 - DNA, 25 bp, Artificial sequence
  Oligonucleotide Probe
tcagcaaggagaccaactgccagac

SEQ ID NO: 265 - DNA, 24 bp, Artificial sequence
  Oligonucleotide Probe
ctgcaggcaatgtgcatccatctg

SEQ ID NO: 266 - DNA, 45 bp, Artificial sequence
  Oligonucleotide Probe
cctcagggctaccgcttccagaagttaagccgagtgttgaatcag